(19) United States
(12) Patent Application Publication
(10) Pub. No.: US 2026/0062426 A1
(43) Pub. Date: Mar. 5, 2026

(54) CYCLIN DEPENDENT KINASE DEGRADERS AND METHODS OF USE THEREOF

(71) Applicant: Differentiated Therapeutics Inc, Carlsbad, CA (US)

(72) Inventors: Timothy A. DWIGHT, Quincy, MA (US); Bryce K. ALLEN, Miami, FL (US)

(21) Appl. No.: 19/311,326

(22) Filed: Aug. 27, 2025

Related U.S. Application Data
(63) Continuation of application No. PCT/US2024/017751, filed on Feb. 28, 2024.
(60) Provisional application No. 63/448,925, filed on Feb. 28, 2023.
(60) Provisional application No. 63/448,915, filed on Feb. 28, 2023.
(60) Provisional application No. 63/467,666, filed on May 19, 2023.

(51) Int. Cl. C07D 519/00; A61K 31/519; A61K 31/55; A61K 31/635; C07D 487/04

(52) U.S. Cl. CPC C07D 519/00; A61K 31/519; A61K 31/55; A61K 31/635; C07D 487/04

(57) ABSTRACT

The present disclosure relates to novel compounds and pharmaceutical compositions thereof, and methods for degrading CDK2 and/or CCNE (CCNE1 and/or CCNE2) with the compounds and compositions of the disclosure. The present disclosure further relates to, but is not limited to, methods for treating disorders associated with CDK2 and/or CCNE (CCNE1 and/or CCNE2) with the compounds and compositions of the disclosure.

### CROSS-REFERENCE TO RELATED APPLICATIONS

This application claims the benefit of PCT/US2024/017751, which claims the benefit of U.S. Provisional Patent Application No. 63/448,915, filed Feb. 28, 2023, U.S. Provisional Patent Application No. 63/448,925, filed Feb. 28, 2023, and U.S. Provisional Patent Application No. 63/467,666, filed May 19, 2023, the entire contents of which are incorporated by reference herein.

### TECHNICAL FIELD OF THE INVENTION

The present invention relates to compounds and methods useful for the modulation of cyclin dependent kinase 2 (CDK2) and/or cyclin E (CCNE1 and/or CCNE2) via ubiquitination and/or degradation by compounds according to the present invention. The invention also provides pharmaceutically acceptable compositions comprising compounds of the present invention and methods of using said compositions in the treatment of various disorders.

### BACKGROUND OF THE INVENTION

Cyclin-dependent kinases (CDKs) are a family of serine/threonine kinases. Heterodimerized with regulatory subunits known as cyclins, CDKs become fully activated and regulate key cellular processes including cell cycle progression and cell division (Morgan, D. O., Annu Rev Cell Dev Biol, 1997. 13: 261-91). Uncontrolled proliferation is a hallmark of cancer cells. The deregulation of the CDK activity is associated with abnormal regulation of cell-cycle, and is detected in virtually all forms of human cancers (Sherr, C. J., Science, 1996. 274(5293): 1672-7).

CDK2 is of particular interest because deregulation of CDK2 activity occurs frequently in a variety of human cancers. CDK2 plays a crucial role in promoting G1/S transition and S phase progression. In complex with cyclin E (CCNE), CDK2 phosphorylates retinoblastoma pocket protein family members (p107, p130, pRb), leading to de-repression of E2F transcription factors, expression of G1/S transition related genes and transition from G1 to S phase (Henley, S. A. and F. A. Dick, Cell Div, 2012, 7(1): p. 10). This in turn enables activation of CDK2/cyclin A, which phosphorylates endogenous substrates that permit DNA synthesis, replication and centrosome duplication (Ekholm, S. V. and S. I. Reed, Curr Opin Cell Biol, 2000. 12(6): 676-84). It has been reported that the CDK2 pathway influences tumorigenesis mainly through amplification and/or overexpression of CCNE1 and mutations that inactivate CDK2 endogenous inhibitors (e.g., p27), respectively (Xu, X., et al., Biochemistry, 1999. 38(27): 8713-22).

CCNE1 copy-number gain and overexpression have been identified in ovarian, gastric, endometrial, breast and other cancers and been associated with poor outcomes in these tumors (Keyomarsi, K., et al., N Engl J Med, 2002. 347(20): 1566-75; Nakayama, N., et al., Cancer, 2010. 116(11): 2621-34; Au-Yeung, G., et al., Clin Cancer Res, 2017. 23(7): 1862-1874; Rosen, D. G., et al., Cancer, 2006. 106(9): 1925-32). Amplification and/or overexpression of CCNE1 also reportedly contribute to trastuzumab resistance in HER2+ breast cancer and resistance to CDK4/6 inhibitors in estrogen receptor-positive breast cancer (Scaltriti, M., et al., Proc Natl Acad Sci USA, 2011. 108(9): 3761-6; Herrera-Abreu, M. T., et al., Cancer Res, 2016. 76(8): 2301-13). Various approaches targeting CDK2 have been shown to induce cell cycle arrest and tumor growth inhibition (Chen, Y N., et al., Proc Natl Acad Sci USA, 1999. 96(8): 4325-9; Mendoza, N., et al., Cancer Res, 2003. 63(5): 1020-4). Inhibition of CDK2 also reportedly restores sensitivity to trastuzumab treatment in resistant HER2+ breast tumors in a preclinical model (Scaltriti, supra).

Ubiquitin-Proteasome Pathway (UPP) is a critical pathway that regulates key regulator proteins and degrades misfolded or abnormal proteins. UPP is central to multiple cellular processes, and if defective or imbalanced, it leads to pathogenesis of a variety of diseases. The covalent attachment of ubiquitin to specific protein substrates is achieved through the action of E3 ubiquitin ligases.

There are over 600 E3 ubiquitin ligases which facilitate the ubiquitination of different proteins in vivo, which can be divided into four families: HECT-domain E3s, U-box E3s, monomeric RING E3s and multi-subunit E3s. See generally Li et al. (PLOS One, 2008, 3, 1487) titled “Genome-wide and functional annotation of human E3 ubiquitin ligases identifies MULAN, a mitochondrial E3 that regulates the organelle's dynamics and signaling.”; Bemdsen et al. (Nat. Struct. Mol. Biol., 2014, 21, 301-307) titled “New insights into ubiquitin E3 ligase mechanism”; Deshaies et al. (Ann. Rev. Biochem., 2009, 78, 399-434) titled “RING domain E3 ubiquitin ligases.”; Spratt et al. (Biochem. 2014, 458, 421-437) titled “RBR E3 ubiquitin ligases: new structures, new insights, new questions.”; and Wang et al. (Nat. Rev. Cancer., 2014, 14, 233-347) titled “Roles of F-box proteins in cancer.”

The UPP is used to induce selective protein degradation, including use of fusion proteins to artificially ubiquitinate target proteins and synthetic small-molecule probes to induce proteasome-dependent degradation. Bifunctional compounds composed of a target protein binding ligand and an E3 ubiquitin ligase ligand induce proteasome-mediated degradation of selected proteins via their recruitment to E3 ubiquitin ligase and subsequent ubiquitination. These drug-like molecules offer the possibility of temporal control over protein expression. Such compounds are capable of inducing the inactivation of a protein of interest upon addition to cells or administration to an animal or human, and could be useful as biochemical reagents and lead to a new paradigm for the treatment of diseases by removing pathogenic or oncogenic proteins (Crews C, Chemistry & Biology, 2010, 17(6):551-555; Schnnekloth J S Jr., Chembiochem, 2005, 6(1):40-46).

An ongoing need exists in the art for effective treatments for disease, especially cancers. As such, small molecule therapeutic agents that leverage UPP mediated protein degradation to target cancer-associated proteins such as cyclin-dependent kinase 2 (“CDK2”), cyclin E (“CCNE1” and/or “CCNE2”) or CDK2 and CCNE(CCNE1 and/or CCNE2) protein hold promise as therapeutic agents. Accordingly, there remains a need to find compounds that are CDK2 degraders, CCNE (CCNE1 and/or CCNE2) degraders or dual CDK2 and CCNE (CCNE1 and/or CCNE2) degraders useful as therapeutic agents.

### SUMMARY OF THE INVENTION

In one aspect of the invention, provided is a compound of Formula A-I:

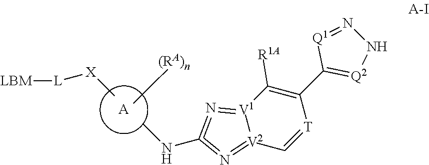

or a pharmaceutically acceptable salt thereof, wherein:
-̅-̅-̅ is a single or a double bond;
Ring A is selected from the group consisting of a nitrogen-containing 4-10 member heterocyclyl, a C₆₋₁₀ aryl and a 5-10-member heteroaryl, wherein the 4-10 member heterocyclyl, C₆₋₁₀ aryl and 5-10-member heteroaryl are attached to the —NH— through a carbon atom;
V¹ is nitrogen and V² is carbon, and Ring

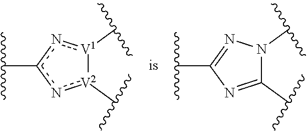

or V² is nitrogen and V¹ is carbon, and Ring

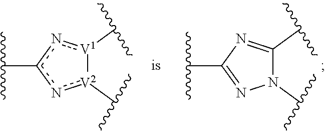

T is CH or N;
Q¹ and Q² are independently selected from N and CH;
R^{1A} is independently selected from H, D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocyclyl, 5-10 membered heteroaryl, C₃₋₁₀ cycloalkyl-C₁₋₄ alkyl-, 6-10 membered aryl-C₁₋₄ alkyl-, 4-10 membered heterocyclyl-C₁₋₄ alkyl-, 5-10 membered heteroaryl-C₁₋₄ alkyl-, OR^{a1}, SR^{a1} NHOR^{a1}, C(O)R^{b1}, C(O)NR^{a1}R^{a1}, C(O)NR^{a1}(OR^{a1}), C(O)OR^{a1}, OC(O)R^{b1}, OC(O)NR^{a1}R^{a1}, NR^{a1}R^{a1}, NR^{a1}NR^{a1}R^{a1}, NR^{a1}C(O)R^{b1}, NR^{a1}C(O)OR^{a1}, NR^{a1}C(O)NR^{a1}R^{a1}, C(═NR^{a1})R^{b1}, C(═NR^{a1})NR^{a1}R^{a1}, NR^{a1}C(═NR^{a1})NR^{a1}R^{a1}, NR^{a1}C(═NR^{a1})R^{b1}, NR^{a1}S(O)NR^{a1}R^{a1}, NR^{a1}S(O)R^{b1}, NR^{a1}S(O)₂R^{b1}, NR^{a1}S(O)(═NR^{a1})R^{b1}, NR^{a1}S(O)₂NR^{a1}R^{a1}, S(O)R^{b1}, S(O)NR^{a1}R^{a1}, S(O)₂R^{b1}, S(O)₂NR^{a1}R^{a1}, OS(O)(═NR^{a1})R^{b1}, OS(O)₂R^{b1}, S(O)(═NR^{a1})R^{b1}, SF₅, P(O)R^{a1}R^{b1}, OP(O)(OR^{a1})(OR^{a1}) and P(O)(OR^{a1})(OR^{a1}), wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocyclyl, 5-10 membered heteroaryl, C₃₋₁₀ cycloalkyl-C₁₋₄ alkyl, 6-10 membered aryl-C₁₋₄ alkyl, 4-10 membered heterocyclyl-C₁₋₄ alkyl, and 5-10 membered heteroaryl-C₁₋₄ alkyl are each substituted with 0, 1, 2, 3, or 4 independently selected R² substituents;
each R² is independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocyclyl, 5-6 membered heteroaryl, C₃₋₇ cycloalkyl-C₁₋₄ alkyl-, phenyl-C₁₋₄ alkyl-, 4-7 membered heterocyclyl-C₁₋₄ alkyl-, 5-6 membered heteroaryl-C₁₋₄ alkyl-, OR^{a2}, SR^{a2}, NHOR^{a2}, C(O)R^{b2}, C(O)NR^{a2}R^{a2}, C(O)NR^{a2}(OR^{a2}), C(O)OR^{a2}, OC(O)R^{b2}, OC(O)NR^{a2}R^{a2}, NR^{a2}R^{a2}, NR^{a2}NR^{a2}R^{a2}, NR^{a2}C(O)R^{b2}, NR^{a2}C(O)OR^{a2}, NR^{a2}C(O)NR^{a2}R^{a2}, C(═NR^{a2})R^{b2}, C(═NR^{a2})NR^{a2}R^{a2}, NR^{a2}C(═NR^{a2})NR^{a2}R^{a2}, NR^{a2}C(═NR^{a2})R^{b2}, NR^{a2}S(O)NR^{a2}R^{a2} NR^{a2}S(O)R^{b2}, NR^{a2}S(O)₂R^{b2}, NR^{a2}S(O)(═NR^{a2})R^{b2}, NR^{a2}S(O)₂NR^{a2}R^{a2}, S(O)R^{b2} S(O)NR^{a2}R^{a2}, S(O)₂R^{b2}, S(O)₂NR^{a2}R^{a2}, OS(O)(═NR^{a2})R^{b2}, OS(O)₂R^{b2}, S(O)(═NR^{a2})R^{b2}, SF₅, P(O)R^{a2}R^{a2}, OP(O)(OR^{a2})(OR^{a2}) and P(O)(OR^{a2})(OR^{a2}), wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocyclyl, 5-6 membered heteroaryl, C₃₋₇ cycloalkyl-C₁₋₄ alkyl-, phenyl-C₁₋₄ alkyl-, 4-7 membered heterocyclyl-C₁₋₄ alkyl-, and 5-6 membered heteroaryl-C₁₋₄ alkyl- are each substituted with 0, 1, 2, 3, or 4 substituents independently selected from C₁₋₄ alkyl, C₃₋₇ cycloalkyl, cyclopropyl, oxo, —C(O)C₁₋₄alkyl, —C(O)OC₁₋₄alkyl, —C(O)NH₂, —OH, —F, —Cl, —O—C₁₋₄alkyl and —CN;
each instance of R^{A} is independently selected from —D, halo, CN, C₁₋₄ alkyl, C₁₋₄ haloalkyl, C₃₋₆ cycloalkyl, 4-6 membered heterocyclyl, OR^{a1}, SR^{a1}, SF₅, NR^{a1}R^{a1}, C₃₋₆ cycloalkyl-C₁₋₃ alkyl-, and 4-6 membered heterocyclyl-C₁₋₃ alkyl-, wherein said C₁₋₄ alkyl, C₁₋₄ haloalkyl-, C₃₋₆ cycloalkyl, 4-6 membered heterocyclyl-, C₃₋₆ cycloalkyl-C₁₋₃ alkyl-, and 4-6 membered heterocyclyl-C₁₋₃ alkyl are substituted with 0, 1, 2, 3, or 4 substituents independently selected from —D, halo, —OH, —C₁₋₄ alkyl and —OC₁₋₄ alkyl; or, alternatively, two R^{A} groups on adjacent atoms of Ring A, together with the ring atoms to which they are attached, form Ring D, wherein Ring D is selected from C₃₋₆ cycloalkyl, 4-6 membered heterocyclyl, phenyl, and 5-6 membered heteroaryl, each of which is substituted with 0, 1, 2, 3, or 4 substituents independently selected from —D, halo, —OH, —C₁₋₄ alkyl and —OC₁₋₄ alkyl;
each R^{a1} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocyclyl, 5-10 membered heteroaryl, C₃₋₁₀ cycloalkyl-C₁₋₄ alkyl-, 6-10 membered aryl-C₁₋₄ alkyl-, 4-10 membered heterocyclyl-C₁₋₄ alkyl-, and 5-10 membered heteroaryl-C₁₋₄ alkyl-, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocyclyl, 5-10 membered heteroaryl, C₃₋₁₀ cycloalkyl-C₁₋₄ alkyl, 6-10 membered aryl-C₁₋₄ alkyl, 4-10 membered heterocyclyl-C₁₋₄ alkyl, and 5-10 membered heteroaryl-C₁₋₄ alkyl are each substituted with 0, 1, 2, 3, or 4 independently selected R² substituents; or two R^{a1} groups attached to the same nitrogen atom together with the nitrogen to which they are attached form a 4-7-membered heterocyclyl group substituted with 0, 1, 2, 3, or 4 substituents independently selected from —D, halo, —OH, —C₁₋₄ alkyl and —OC₁₋₄ alkyl; each R^{b1} is independently selected from C₁₋₆ alkyl, C₁₋₆ hydroxyalkyl, C₃₋₉ cycloalkyl and C₂₋₆ heteroalkyl;
each R^{a2} is independently selected from H, C₁₋₆ alkyl, C₁₋₆ hydroxyalkyl, C₃₋₉ cycloalkyl and C₂₋₆ heteroalkyl, or, when possible, two instances of R^{a2} and the atom to which they are attached are taken together to form a 4-7 member heterocycle substituted with 0, 1, 2, 3, or 4 substituents independently selected from —D, halo, —OH, —C₁₋₄ alkyl and —OC₁₋₄ alkyl;
each R^{b2} is independently selected from C₁₋₆ alkyl, C₁₋₆ hydroxyalkyl, C₃₋₉ cycloalkyl and C₂₋₆ heteroalkyl;
X is X¹ when Ring A is heterocyclyl and is selected from X¹ and X² when Ring A is aryl or heteroaryl;
X¹ is selected from —S(O)₂— and —C(O)—;
X² is selected from —O—, —NH—, —N(CH₃)— and —CH₂—;
L is a covalent bond or a bivalent, saturated or unsaturated, straight or branched C₁₋₅₀ hydrocarbon chain, wherein 0-6 methylene units of L are independently replaced by —Cy—, —CH(R)—, —C(R)₂—, —O—, —NR—, —S—, —OC(═O)—, —C(═O)O—, —C(═O)—, —S(═O)—, —S(═O)₂—, —NRS(═O)₂—, —S(═O)₂NR—, —NRC(═O)—, —C(═O)NR—, —OC(═O)NR— or —NRC(═O)O—, wherein:
each —Cy— is independently a bivalent ring selected from phenylene, an 8-10 membered bicyclic arylene, a 4-7 membered monocyclic carbocyclylene, a 5-11 membered spiro carbocyclylene, a 4-10 membered bicyclic carbocyclylene, a 5-10 membered bridged carbocyclylene, a 4-7 membered monocyclic heterocyclylene having 1-2 heteroatoms independently selected from nitrogen, oxygen, and sulfur, a 5-11 membered spiro heterocyclylene having 1-2 heteroatoms independently selected from nitrogen, oxygen, and sulfur, an 4-10 membered bicyclic heterocyclylene having 1-2 heteroatoms independently selected from nitrogen, oxygen, and sulfur, a 5-10 membered bridged bicyclic saturated or partially unsaturated heterocyclylene having 1-2 heteroatoms independently selected from nitrogen, oxygen, and sulfur, a 5-6 membered heteroarylene having 1-4 heteroatoms independently selected from nitrogen, oxygen, and sulfur, or an 8-10 membered bicyclic heteroarylene having 1-5 heteroatoms independently selected from nitrogen, oxygen, or sulfur, wherein each phenylene, arylene, carbocyclylene, heterocyclylene and heteroarylene is substituted with 0, 1, 2, 3, or 4 instances of R^{C};
LBM is selected from:

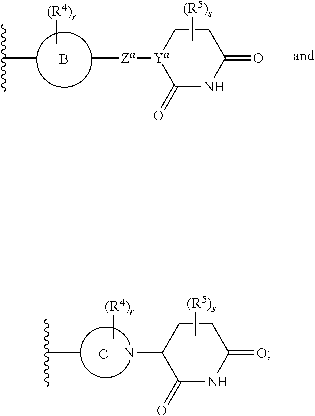

Y^{a} is CH or N;
Z^{a} is a bond, —CH₂—, —NH—, 0, or —NHC(O)— where NH of —NHC(O)— is attached to Y^{a};
Ring B is phenylene, a 4-10-membered heterocyclylene, a 5-6-membered monocyclic heteroarylene or a 9-10-membered fused bicyclic heteroarylene, wherein each heteroarylene contains one to three nitrogen ring atoms.
ring C together with the (R⁴)r substituents is selected from the group consisting of:

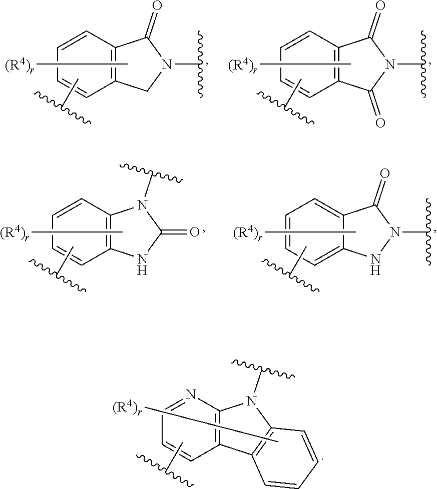

each instance of R^{C} is independently selected from —D, halogen, —OH, and —C₁₋₆ alkyl;
each instance of R⁴ is independently selected from —D, halogen, —OH, and —C₁₋₆ alkyl;
each instance of R⁵ is independently selected from —D, halogen, —OH, and —C₁₋₆ alkyl;
each instance of R is independently selected from hydrogen and —C₁₋₆ alkyl;
n is 0, 1, 2, 3, or 4;
r is 0, 1, 2, 3, or 4; and
s is 0, 1, 2, 3, or 4.

In one aspect of the invention, provided is a compound of Formula I:

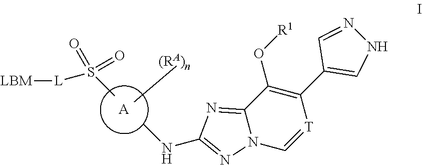

or a pharmaceutically acceptable salt thereof, wherein:
Ring A is selected from

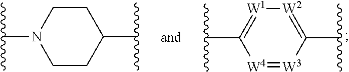

W¹, W², W³ and W⁴ are each independently CH or N;
R¹ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocycloalkyl, 5-10 membered heteroaryl, C₃₋₁₀ cycloalkyl-C₁₋₄ alkyl-, 6-10 membered aryl-C₁₋₄ alkyl-, 4-10 membered heterocyclyl-C₁₋₄ alkyl-, and 5-10 membered heteroaryl-C₁₋₄ alkyl-, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocyclyl, 5-10 membered heteroaryl, C₃₋₁₀ cycloalkyl-C₁₋₄ alkyl, 6-10 membered aryl-C₁₋₄ alkyl, 4-10 membered heterocyclyl-C₁₋₄ alkyl, and 5-10 membered heteroaryl-C₁₋₄ alkyl are each substituted with 0, 1, 2, 3, or 4 independently selected R² substituents;
each R² is independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocyclyl, 5-6 membered heteroaryl, C₃₋₇ cycloalkyl-C₁₋₄ alkyl-, phenyl-C₁₋₄ alkyl-, 4-7 membered heterocyclyl-C₁₋₄ alkyl-, 5-6 membered heteroaryl-C₁₋₄ alkyl-, OR^{a2}, SR^{a2}, NHOR^{a2}, C(O)R^{b2}, C(O)NR^{a2}R^{a2}, C(O)NR^{a2}(OR^{a2}), C(O)OR^{a2}, OC(O)R^{b2}, OC(O)NR^{a2}R^{a2}, NR^{a2}R^{a2}, NR^{a2}NR^{a2}R^{a2}, NR^{a2}C(O)R^{b2}, NR^{a2}C(O)OR^{a2}, NR^{a2}C(O)NR^{a2}R^{a2}, C(═NR^{a2})R^{b2}, C(═NR^{a2})NR^{a2}R^{a2}, NR^{a2}C(═NR^{a2})NR^{a2}R^{a2}, NR^{a2}C(═NR^{a2})R^{b2}, NR^{a2}S(O)NR^{a2}R^{a2}, NR^{a2}S(O)R^{b2}, NR^{a2}S(O)₂R^{b2}, NR^{a2}S(O)(═NR^{a2})R^{b2}, NR^{a2}S(O)₂NR^{a2}R^{a2}, S(O)R^{b2}, S(O)NR^{a2}R^{a2}, S(O)₂R^{b2}, S(O)₂NR^{a2}R^{a2}, OS(O)(═NR^{a2})R^{b2}, OS(O)₂R^{b2}, S(O)(═NR^{a2})R^{b2}, SF₅, P(O)R^{b2}R^{b2}, OP(O)(OR^{a2})(OR^{a2}) and P(O)(OR^{a2})(OR^{a2}), wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocyclyl, 5-6 membered heteroaryl, C₃₋₇ cycloalkyl-C₁₋₄ alkyl, phenyl-C₁₋₄ alkyl, 4-7 membered heterocyclyl-C₁₋₄ alkyl, and 5-6 membered heteroaryl-C₁₋₄ alkyl are each substituted with 0, 1, 2, 3, or 4 substituents independently selected from C₁₋₄ alkyl, C₃₋₇ cycloalkyl, cyclopropyl, oxo, —C(O)C₁₋₄alkyl, —C(O)OC₁₋₄alkyl, —C(O)NH₂, —OH, —F, —Cl, —O—C₁₋₄alkyl and —CN;
each R^{a2} is independently selected from H, C₁₋₆ alkyl, C₁₋₆ hydroxyalkyl, C₃₋₉ cycloalkyl and C₂₋₆ heteroalkyl, or, when possible, two instances of R^{a2} and the atom to which they are attached are taken together to form a saturated 3-7-membered heterocycle;
each R^{b2} is independently selected from C₁₋₆ alkyl, C₁₋₆ hydroxyalkyl, C₃₋₉ cycloalkyl and C₂₋₆ heteroalkyl;
L is a covalent bond or a bivalent, saturated or unsaturated, straight or branched C₁₋₅₀ hydrocarbon chain, wherein 0-6 methylene units of L are independently replaced by —Cy—, —CH(R)—, —C(R)₂—, —O—, —NR—, —S—, —OC(═O)—, —C(═O)O—, —C(═O)—, —S(═O)—, —S(═O)₂—, —NRS(═O)₂—, —S(═O)₂NR—, —NRC(═O)—, —C(═O)NR—, —OC(═O)NR— or —NRC(═O)O—, wherein:
each —Cy— is independently a bivalent ring selected from phenylene, an 8-10 membered bicyclic arylene, a 4-7 membered monocyclic carbocyclylene, a 5-11 membered spiro carbocyclylene, a 4-10 membered bicyclic carbocyclylene, a 5-10 membered bridged carbocyclylene, a 4-7 membered monocyclic heterocyclylene having 1-2 heteroatoms independently selected from nitrogen, oxygen, and sulfur, a 5-11 membered spiro heterocyclylene having 1-2 heteroatoms independently selected from nitrogen, oxygen, and sulfur, an 4-10 membered bicyclic heterocyclylene having 1-2 heteroatoms independently selected from nitrogen, oxygen, and sulfur, a 5-10 membered bridged bicyclic saturated or partially unsaturated heterocyclylene having 1-2 heteroatoms independently selected from nitrogen, oxygen, and sulfur, a 5-6 membered heteroarylene having 1-4 heteroatoms independently selected from nitrogen, oxygen, and sulfur, or an 8-10 membered bicyclic heteroarylene having 1-5 heteroatoms independently selected from nitrogen, oxygen, or sulfur, wherein each phenylene, arylene, carbocyclylene, heterocyclylene and heteroarylene is substituted with 0, 1, 2, 3, or 4 instances of Re;
LBM is selected from:

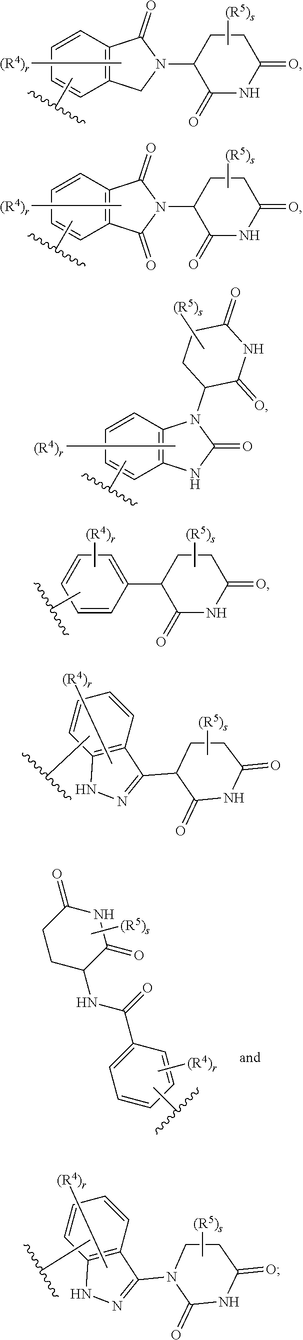

each instance of R^{A} is independently selected from —D, halogen, —C₁₋₆ alkyl, —OH and —OC₁₋₄ alkyl, wherein each —C₁₋₆ alkyl is substituted with 0, 1, 2 or 3 groups independently selected from —D, —F, —OH and —OC₁₋₄ alkyl; or two R^{A} groups on adjacent atoms of Ring A, together with the ring atoms to which they are attached, form Ring D, wherein Ring D is selected from C₃₋₆ cycloalkyl, 4-6 membered heterocyclyl, phenyl, and 5-6 membered heteroaryl, each of which is substituted with 0, 1, 2, 3, or 4 substituents independently selected from —D, halo, —OH, —C₁₋₄ alkyl and —OC₁₋₄ alkyl;
each instance of R^{C} is independently selected from —D, halogen, —OH, and —C₁₋₆ alkyl;
each instance of R⁴ is independently selected from —D, halogen, —OH, and —C₁₋₆ alkyl;
each instance of R⁵ is independently selected from —D, halogen, —OH, and —C₁₋₆ alkyl;
each instance of R is independently selected from hydrogen and —C₁₋₆ alkyl;
n is 0,1,2,3, or 4;
r is 0, 1, 2, 3, or 4; and
s is 0, 1, 2, 3, or 4.

In an embodiment, provided is a pharmaceutical composition comprising a compound of Formula A-I or Formula I as described herein, or a pharmaceutically acceptable salt thereof, and a pharmaceutically acceptable carrier, adjuvant, or diluent.

In an embodiment, provided is a method of inhibiting CDK2 and/or CCNE (CCNE1 and/or CCNE2) signaling in a sample, e.g., in vivo or in vitro, by contacting CDK2 and/or CCNE (CCNE1 and/or CCNE2) with a compound or pharmaceutically acceptable salt thereof or composition as described herein.

In an embodiment, provided is a use of a compound or pharmaceutically acceptable salt thereof or composition as described herein in a method of inhibiting CDK2 and/or CCNE (CCNE1 and/or CCNE2) signaling in a sample, e.g., in vivo or in vitro, by contacting CDK2 and/or CCNE (CCNE1 and/or CCNE2) with the compound or composition.

In an embodiment, provided is a use of a compound or pharmaceutically acceptable salt thereof or composition as described herein in the manufacturing of a medicament for inhibiting CDK2 and/or CCNE (CCNE1 and/or CCNE2) signaling in a sample, e.g., in vivo or in vitro.

In an embodiment, provided is a compound or pharmaceutically acceptable salt thereof or composition as described herein for use in a method of inhibiting CDK2 and/or CCNE (CCNE1 and/or CCNE2) signaling in a sample, e.g., in vivo or in vitro, by contacting CDK2 and/or CCNE (CCNE1 and/or CCNE2) with the compound or composition.

In an embodiment, provided is a compound or pharmaceutically acceptable salt thereof or composition as described herein for use in manufacturing of a medicament for inhibiting CDK2 and/or CCNE (CCNE1 and/or CCNE2) signaling in a sample, e.g., in vivo or in vitro, by contacting CDK2 and/or CCNE (CCNE1 and/or CCNE2) with the compound or composition.

In an embodiment, provided is a method of inhibiting CDK2 signaling in a sample, e.g., in vivo or in vitro, by contacting CDK2 with a compound or pharmaceutically acceptable salt thereof or composition as described herein.

In an embodiment, provided is a use of a compound or pharmaceutically acceptable salt thereof or composition as described herein in a method of inhibiting CDK2 signaling in a sample, e.g., in vivo or in vitro, by contacting CDK2 with a compound or pharmaceutically acceptable salt thereof or composition as described herein.

In an embodiment, provided is a use of a compound or pharmaceutically acceptable salt thereof or composition as described herein in the manufacturing of a medicament for inhibiting CDK2 signaling in a sample, e.g., in vivo or in vitro.

In an embodiment, provided is a compound or pharmaceutically acceptable salt thereof or composition as described herein for use in a method of inhibiting CDK2 signaling in a sample, e.g., in vivo or in vitro, by contacting CDK2 with a compound or pharmaceutically acceptable salt thereof or composition as described herein.

In an embodiment, provided is a compound or pharmaceutically acceptable salt thereof or composition as described herein for use in the manufacturing of a medicament for inhibiting CDK2 signaling in a sample, e.g., in vivo or in vitro.

In an embodiment, provided is a method of inhibiting CCNE (CCNE1 and/or CCNE2) signaling in a sample, e.g., in vivo or in vitro, by contacting CCNE (CCNE1 and/or CCNE2) with a compound or pharmaceutically acceptable salt thereof or composition as described herein.

In an embodiment, provided is a use of a compound or pharmaceutically acceptable salt thereof or composition as described herein in a method of inhibiting CCNE (CCNE1 and/or CCNE2) signaling in a sample, e.g., in vivo or in vitro, by contacting CCNE (CCNE1 and/or CCNE2) with a compound or pharmaceutically acceptable salt thereof or composition as described herein.

In an embodiment, provided is a use of a compound or pharmaceutically acceptable salt thereof or composition as described herein in the manufacturing of a medicament for inhibiting CCNE (CCNE1 and/or CCNE2) signaling in a sample, e.g., in vivo or in vitro.

In an embodiment, provided is a compound or pharmaceutically acceptable salt thereof or composition as described herein for use in a method of inhibiting CCNE (CCNE1 and/or CCNE2) signaling in a sample, e.g., in vivo or in vitro, by contacting CCNE (CCNE1 and/or CCNE2) with a compound or pharmaceutically acceptable salt thereof or composition as described herein.

In an embodiment, provided is a compound or pharmaceutically acceptable salt thereof or composition as described herein for use in the manufacturing of a medicament for inhibiting CCNE (CCNE1 and/or CCNE2) signaling in a sample, e.g., in vivo or in vitro.

In an embodiment, provided is a method of inhibiting CDK2 and CCNE (CCNE1 and/or CCNE2) signaling in a sample, e.g., in vivo or in vitro, by contacting CDK2 and/or CCNE (CCNE1 and/or CCNE2) with a compound or pharmaceutically acceptable salt thereof or composition as described herein.

In an embodiment, provided is a use of a compound or pharmaceutically acceptable salt thereof or composition as described herein in a method of inhibiting CDK2 and CCNE (CCNE1 and/or CCNE2) signaling in a sample, e.g., in vivo or in vitro, by contacting CDK2 and/or CCNE (CCNE1 and/or CCNE2) with a compound or pharmaceutically acceptable salt thereof or composition as described herein.

In an embodiment, provided is a use of a compound or pharmaceutically acceptable salt thereof or composition as described herein in the manufacturing of a medicament for inhibiting CDK2 and CCNE (CCNE1 and/or CCNE2) signaling in a sample, e.g., in vivo or in vitro.

In an embodiment, provided is a compound or pharmaceutically acceptable salt thereof or composition as described herein for use in a method of inhibiting CDK2 and CCNE (CCNE1 and/or CCNE2) signaling in a sample, e.g., in vivo or in vitro, by contacting CDK2 and/or CCNE (CCNE1 and/or CCNE2) with a compound or pharmaceutically acceptable salt thereof or composition as described herein.

In an embodiment, provided is a compound or pharmaceutically acceptable salt thereof or composition as described herein for use in the manufacturing of a medicament for inhibiting CDK2 and CCNE (CCNE1 and/or CCNE2) signaling in a sample, e.g., in vivo or in vitro.

In an embodiment, provided is a method of treating a CDK2 and/or CCNE (CCNE1 and/or CCNE2)-mediated disorder in a patient in need thereof, comprising administering to the patient a compound or pharmaceutically acceptable salt thereof or composition as described herein.

In an embodiment, provided is a use of a compound or pharmaceutically acceptable salt thereof or composition as described herein in a method of treating a CDK2 and/or CCNE (CCNE1 and/or CCNE2)-mediated disorder in a patient in need thereof, comprising administering to the patient a compound or pharmaceutically acceptable salt thereof or composition as described herein.

In an embodiment, provided is a use of a compound or pharmaceutically acceptable salt thereof or composition as described herein in the manufacturing of a medicament for treating a CDK2 and/or CCNE (CCNE1 and/or CCNE2)-mediated disorder in a patient in need thereof.

In an embodiment, provided is a compound or pharmaceutically acceptable salt thereof or composition as described herein for use in a method of treating a CDK2 and/or CCNE (CCNE1 and/or CCNE2)-mediated disorder in a patient in need thereof, comprising administering to the patient a compound or pharmaceutically acceptable salt thereof or composition as described herein.

In an embodiment, provided is a compound or pharmaceutically acceptable salt thereof or composition as described herein for use in the manufacturing of a medicament for treating a CDK2 and/or CCNE (CCNE1 and/or CCNE2)-mediated disorder in a patient in need thereof.

In an embodiment, provided is a method of treating a CDK2-mediated disorder in a patient in need thereof, comprising administering to the patient a compound or pharmaceutically acceptable salt thereof or composition as described herein.

In an embodiment, provided is a use of a compound or pharmaceutically acceptable salt thereof or composition as described herein in a method of treating a CDK2-mediated disorder in a patient in need thereof, comprising administering to the patient a compound or pharmaceutically acceptable salt thereof or composition as described herein.

In an embodiment, provided is a use of a compound or pharmaceutically acceptable salt thereof or composition as described herein in the manufacturing of a medicament for treating a CDK2-mediated disorder in a patient in need thereof.

In an embodiment, provided is a compound or pharmaceutically acceptable salt thereof or composition as described herein for use in a method of treating a CDK2-mediated disorder in a patient in need thereof, comprising administering to the patient a compound or pharmaceutically acceptable salt thereof or composition as described herein.

In an embodiment, provided is a compound or pharmaceutically acceptable salt thereof or composition as described herein for use in the manufacturing of a medicament for treating a CDK2-mediated disorder in a patient in need thereof.

In an embodiment, provided is a method of treating a CCNE (CCNE1 and/or CCNE2)-mediated disorder in a patient in need thereof, comprising administering to the patient a compound or pharmaceutically acceptable salt thereof or composition as described herein.

In an embodiment, provided is a use of a compound or pharmaceutically acceptable salt thereof or composition as described herein in a method of treating a CCNE (CCNE1 and/or CCNE2)-mediated disorder in a patient in need thereof, comprising administering to the patient a compound or pharmaceutically acceptable salt thereof or composition as described herein.

In an embodiment, provided is a use of a compound or pharmaceutically acceptable salt thereof or composition as described herein in the manufacturing of a medicament for treating a CCNE (CCNE1 and/or CCNE2)-mediated disorder in a patient in need thereof.

In an embodiment, provided is a compound or pharmaceutically acceptable salt thereof or composition as described herein for use in a method of treating a CCNE (CCNE1 and/or CCNE2)-mediated disorder in a patient in need thereof, comprising administering to the patient a compound or pharmaceutically acceptable salt thereof or composition as described herein.

In an embodiment, provided is a compound or pharmaceutically acceptable salt thereof or composition as described herein for use in the manufacturing of a medicament for treating a CCNE (CCNE1 and/or CCNE2)-mediated disorder in a patient in need thereof.

In an embodiment, provided is a method of treating a CDK2 and CCNE (CCNE1 and/or CCNE2)-mediated disorder in a patient in need thereof, comprising administering to the patient a compound or pharmaceutically acceptable salt thereof or composition as described herein.

In an embodiment, provided is a use of a compound or pharmaceutically acceptable salt thereof or composition as described herein in a method of treating a CDK2 and CCNE (CCNE1 and/or CCNE2)-mediated disorder in a patient in need thereof, comprising administering to the patient a compound or pharmaceutically acceptable salt thereof or composition as described herein.

In an embodiment, provided is a use of a compound or pharmaceutically acceptable salt thereof or composition as described herein in the manufacturing of a medicament for treating a CDK2 and CCNE (CCNE1 and/or CCNE2)-mediated disorder in a patient in need thereof.

In an embodiment, provided is compound or pharmaceutically acceptable salt thereof or composition as described herein for use in a method of treating a CDK2 and CCNE (CCNE1 and/or CCNE2)-mediated disorder in a patient in need thereof, comprising administering to the patient a compound or pharmaceutically acceptable salt thereof or composition as described herein.

In an embodiment, provided is a compound or pharmaceutically acceptable salt thereof or composition as described herein for use in the manufacturing of a medicament for treating a CDK2 and CCNE (CCNE1 and/or CCNE2)-mediated disorder in a patient in need thereof.

### DETAILED DESCRIPTION

### Definitions

As used in the present disclosure, the following words and phrases are generally intended to have the meanings as set forth below unless expressly indicated otherwise or the context in which they are used indicates otherwise.

Unless defined otherwise, all technical and scientific terms used herein have the same meaning as commonly understood by one of ordinary skill in the art to which this invention belongs. The abbreviations used herein have their conventional meaning within the chemical and biological arts. The chemical structures and formulae set forth herein are constructed according to the standard rules of chemical valency known in the chemical arts.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. Where elements are presented as lists, e.g., in Markush group format, each subgroup of the elements is also disclosed, and any element(s) can be removed from the group. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

The articles “a” and “an” are used in this disclosure to refer to one or more than one (i.e., to at least one) of the grammatical object of the article, unless the context is inappropriate. By way of example, in certain contexts, “an element” means one element and/or in certain contexts more than one element. By way of another example, in certain contexts “a compound” means one compound and/or in certain contexts more than one compound (e.g., a mixture of two or more compounds).

The term “and/or” is used in this disclosure to mean either “and” or “or” unless indicated otherwise.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a±10% variation from the nominal value unless otherwise indicated or inferred from the context.

At various places in the present specification, variables or parameters are disclosed in groups or in ranges. It is specifically intended that the description include each and every individual subcombination of the members of such groups and ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### Chemical Definitions

These and other exemplary substituents are described in more detail in the Detailed Description, Examples, and Claims. The invention is not intended to be limited in any manner by the above exemplary listing of substituents.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). Additionally encompassed are compounds described herein as individual isomers substantially free of other isomers, and alternatively, as mixtures of various isomers.

The “enantiomeric excess” (“e.e.”) or “% enantiomeric excess” (“% e.e.”) of a composition as used herein refers to an excess of one enantiomer relative to the other enantiomer present in the composition. For example, a composition can contain 90% of one enantiomer, e.g., the S enantiomer, and 10% of the other enantiomer, i.e., the R enantiomer.

$$e . e . = ( 90 - 10 ) / 100 = 80 \% .$$

Thus, a composition containing 90% of one enantiomer and 10% of the other enantiomer is said to have an enantiomeric excess of 80%.

The “diastereomeric excess” (“d.e.”) or “% diastereomeric excess” (“% d.e.”) of a composition as used herein refers to an excess of one diastereomer relative to one or more different diastereomers present in the composition. For example, a composition can contain 90% of one diastereomer, and 10% of one or more different diastereomers.

$$d . e . = ( 90 - 10 ) / 100 = 80 \% .$$

Thus, a composition containing 90% of one diastereomers and 10% of one or more different diastereomers is said to have a diastereomeric excess of 80%.

In an alternative embodiment, compounds described herein may also comprise one or more isotopic substitutions. For example, hydrogen may be ²H (D or deuterium) or ³H (T or tritium); carbon may be, for example, ¹³C or ¹⁴C; oxygen may be, for example, 180; nitrogen may be, for example, ¹⁵N, and the like. In other embodiments, a particular isotope (e.g., ³H, ¹³C, ¹⁴C, ¹⁸O, or ¹⁵N) can represent at least 1%, at least 5%, at least 10%, at least 15%, at least 20%, at least 25%, at least 30%, at least 35%, at least 40%, at least 45%, at least 50%, at least 60%, at least 65%, at least 70%, at least 75%, at least 80%, at least 85%, at least 90%, at least 95%, at least 99%, or at least 99.9% of the total isotopic abundance of an element that occupies a specific site of the compound.

In a formula, [symbol] is a single bond where the stereochemistry of the moieties [symbol] immediately attached thereto is not specified.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein. The articles “a” and “an” may be used herein to refer to one or to more than one (i.e. at least one) of the grammatical objects of the article. By way of example “an analogue” means one analogue or more than one analogue.

The term “unsaturated bond” refers to a double or triple bond.

The term “unsaturated” or “partially unsaturated” refers to a moiety that includes at least one double or triple bond.

The term “saturated” refers to a moiety that does not contain a double or triple bond, i.e., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

The term “azido” refers to the radical —N₃.

“Aliphatic” refers to an alkyl, alkenyl, alkynyl, or carbocyclyl group, as defined herein.

“Cycloalkylalkyl” refers to an alkyl radical in which the alkyl group is substituted with a cycloalkyl group. Typical cycloalkylalkyl groups include, but are not limited to, cyclopropylmethyl, cyclobutylmethyl, cyclopentylmethyl, cyclohexylmethyl, cycloheptylmethyl, cyclooctylmethyl, cyclopropylethyl, cyclobutylethyl, cyclopentylethyl, cyclohexylethyl, cycloheptylethyl, and cyclooctylethyl, and the like.

“Heterocyclylalkyl” refers to an alkyl radical in which the alkyl group is substituted with a heterocyclyl group. Typical heterocyclylalkyl groups include, but are not limited to, pyrrolidinylmethyl, piperidinylmethyl, piperazinylmethyl, morpholinylmethyl, pyrrolidinylethyl, piperidinylethyl, piperazinylethyl, morpholinylethyl, and the like.

“Aralkyl” or “arylalkyl” is a subset of alkyl and aryl, as defined herein, and refers to an optionally substituted alkyl group substituted by an optionally substituted aryl group.

“Alkyl” refers to a radical of a straight-chain or branched saturated hydrocarbon group having from 1 to 20 carbon atoms (“C₁₋₂₀ alkyl”). In an embodiment, an alkyl group has 1 to 12 carbon atoms (“C₁₋₁₂ alkyl”). In an embodiment, an alkyl group has 1 to 10 carbon atoms (“C₁₋₁₀ alkyl”). In an embodiment, an alkyl group has 1 to 9 carbon atoms (“C₁₋₉ alkyl”). In an embodiment, an alkyl group has 1 to 8 carbon atoms (“C₁₋₈ alkyl”). In an embodiment, an alkyl group has 1 to 7 carbon atoms (“C₁₋₇ alkyl”). In an embodiment, an alkyl group has 1 to 6 carbon atoms (“C₁₋₆ alkyl”, also referred to herein as “lower alkyl”). In an embodiment, an alkyl group has 1 to 5 carbon atoms (“C₁₋₅ alkyl”). In an embodiment, an alkyl group has 1 to 4 carbon atoms (“C₁₋₄ alkyl”). In an embodiment, an alkyl group has 1 to 3 carbon atoms (“C₁₋₃ alkyl”). In an embodiment, an alkyl group has 1 to 2 carbon atoms (“C₁₋₂ alkyl”). In an embodiment, an alkyl group has 1 carbon atom (“C₁ alkyl”). In an embodiment, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C). Additional examples of alkyl groups include n-heptyl (C₇), n-octyl (C₈) and the like. Unless otherwise specified, each instance of an alkyl group is independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁₋₁₀ alkyl (e.g., —CH₃). In certain embodiments, the alkyl group is substituted C₁₋₁₀ alkyl. Common alkyl abbreviations include Me(—CH₃), Et (—CH₂CH₃), ′Pr(—CH(CH₃)₂), ″Pr(—CH₂CH₂CH₃), ″Bu (—CH₂CH₂CH₂CH₃), or ′Bu (—CH₂CH(CH₃)₂).

“Alkylene” refers to an alkyl group wherein two hydrogens are removed to provide a divalent radical, and which may be substituted or unsubstituted. Unsubstituted alkylene groups include, but are not limited to, methylene (—CH₂—), ethylene (—CH₂CH₂—), propylene (—CH₂CH₂CH₂—), butylene (—CH₂CH₂CH₂CH₂—), pentylene (—CH₂CH₂CH₂CH₂CH₂—), hexylene (—CH₂CH₂CH₂CH₂CH₂CH₂—), and the like. Exemplary substituted alkylene groups, e.g., substituted with one or more alkyl (methyl) groups, include but are not limited to, substituted methylene (—CH(CH₃)—, (—C(CH₃)₂—), substituted ethylene (—CH(CH₃)CH₂—, —CH₂CH(CH₃)—, —C(CH₃)₂CH₂—, —CH₂C(CH₃)₂—), substituted propylene (—CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—), and the like. When a range or number of carbons is provided for a particular alkylene group, it is understood that the range or number refers to the range or number of carbons in the linear carbon divalent chain. Alkylene groups may be substituted or unsubstituted with one or more substituents as described herein.

“Alkenyl” refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds) (“C₂₋₂₀ alkenyl”). In certain embodiments, alkenyl does not contain any triple bonds. In an embodiment, an alkenyl group has 2 to 10 carbon atoms (“C₂₋₁₀ alkenyl”). In an embodiment, an alkenyl group has 2 to 9 carbon atoms (“C₂₋₉ alkenyl”). In an embodiment, an alkenyl group has 2 to 8 carbon atoms (“C₂₋₈ alkenyl”). In an embodiment, an alkenyl group has 2 to 7 carbon atoms (“C₂₋₇ alkenyl”). In an embodiment, an alkenyl group has 2 to 6 carbon atoms (“C₂₋₆ alkenyl”). In an embodiment, an alkenyl group has 2 to 5 carbon atoms (“C₂₋₅ alkenyl”). In an embodiment, an alkenyl group has 2 to 4 carbon atoms (“C₂₋₄ alkenyl”). In an embodiment, an alkenyl group has 2 to 3 carbon atoms (“C₂₋₃ alkenyl”). In an embodiment, an alkenyl group has 2 carbon atoms (“C₂alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₂₋₁₀ alkenyl. In certain embodiments, the alkenyl group is substituted C₂₋₁₀ alkenyl.

“Alkynyl” refers to a radical of a straight-chain or branched hydrocarbon group having from 2 to 20 carbon atoms, one or more carbon-carbon triple bonds (e.g., 1, 2, 3, or 4 carbon-carbon triple bonds), and optionally one or more carbon-carbon double bonds (e.g. 1, 2, 3, or 4 carbon-carbon double bonds) (“C₂₋₂₀ alkynyl”). In certain embodiments, alkynyl does not contain any double bonds. In an embodiment, an alkynyl group has 2 to 10 carbon atoms (“C₂₋₁₀ alkynyl”). In an embodiment, an alkynyl group has 2 to 9 carbon atoms (“C₂₋₉ alkynyl”). In an embodiment, an alkynyl group has 2 to 8 carbon atoms (“C₂₋₈ alkynyl”). In an embodiment, an alkynyl group has 2 to 7 carbon atoms (“C₂₋₇ alkynyl”). In an embodiment, an alkynyl group has 2 to 6 carbon atoms (“C₂₋₆ alkynyl”). In an embodiment, an alkynyl group has 2 to 5 carbon atoms (“C₂₋₅ alkynyl”). In an embodiment, an alkynyl group has 2 to 4 carbon atoms (“C₂₋₄ alkynyl”). In an embodiment, an alkynyl group has 2 to 3 carbon atoms (“C₂₋₃ alkynyl”). In an embodiment, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C), and the like. Additional examples of alkynyl include heptynyl (C₇), octynyl (C₈), and the like. Unless otherwise specified, each instance of an alkynyl group is independently optionally substituted, i.e., unsubstituted (an “unsubstituted alkynyl”) or substituted (a “substituted alkynyl”) with one or more substituents; e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₁₀ alkynyl.

The term “heteroalkyl,” as used herein, refers to an alkyl group, as defined herein, which further comprises 1 or more (e.g., 1, 2, 3, or 4) heteroatoms (e.g., oxygen, sulfur, nitrogen, boron, silicon, phosphorus) within the parent chain, wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkyl group refers to a saturated group having from 1 to 10 carbon atoms and 1, 2, 3, or 4 heteroatoms (“heteroC₁₋₁₀ alkyl”). In an embodiment, a heteroalkyl group is a saturated group having 1 to 9 carbon atoms and 1, 2, 3, or 4 heteroatoms (“heteroC₁₋₉ alkyl”). In an embodiment, a heteroalkyl group is a saturated group having 1 to 8 carbon atoms and 1, 2, 3, or 4 heteroatoms (“heteroC₁₋₈ alkyl”). In an embodiment, a heteroalkyl group is a saturated group having 1 to 7 carbon atoms and 1, 2, 3, or 4 heteroatoms (“heteroCₗ-7 alkyl”). In an embodiment, a heteroalkyl group is a group having 1 to 6 carbon atoms and 1, 2, or 3 heteroatoms (“heteroC₁₋₆ alkyl”). In an embodiment, a heteroalkyl group is a saturated group having 1 to 5 carbon atoms and 1 or 2 heteroatoms (“heteroCₗ-5 alkyl”). In an embodiment, a heteroalkyl group is a saturated group having 1 to 4 carbon atoms and 1 or 2 heteroatoms (“heteroC₁₋₄ alkyl”). In an embodiment, a heteroalkyl group is a saturated group having 1 to 3 carbon atoms and 1 heteroatom (“heteroC₁₋₃ alkyl”). In an embodiment, a heteroalkyl group is a saturated group having 1 to 2 carbon atoms and 1 heteroatom (“heteroC₁₋₂ alkyl”). In an embodiment, a heteroalkyl group is a saturated group having 1 carbon atom and 1 heteroatom (“heteroC₁ alkyl”). In an embodiment, a heteroalkyl group is a saturated group having 2 to 6 carbon atoms and 1 or 2 heteroatoms (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl. Exemplary heteroalkyl groups include: —CH₂OH, —CH₂OCH₃, —CH₂NH₂, —CH₂NH(CH₃), —CH₂N(CH₃)₂, —CH₂CH₂OH, —CH₂CH₂OCH₃, —CH₂CH₂NH₂, —CH₂CH₂NH(CH₃), —CH₂CH₂N(CH₃)₂.

“Aryl” refers to a radical of a monocyclic or polycyclic (e.g., bicyclic or tricyclic) 4n+2 aromatic ring system (e.g., having 6, 10, or 14 1L electrons shared in a cyclic array) having 6-14 ring carbon atoms and zero heteroatoms provided in the aromatic ring system (“C₆₋₁₄ aryl”). In an embodiment, an aryl group has six ring carbon atoms (“C₆ aryl”; e.g., phenyl). In an embodiment, an aryl group has ten ring carbon atoms (“C₁₀ aryl”; e.g., naphthyl such as 1-naphthyl and 2-naphthyl). In an embodiment, an aryl group has fourteen ring carbon atoms (“C₁₋₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Particularly aryl groups include phenyl, naphthyl, indenyl, and tetrahydronaphthyl. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

In certain embodiments, an aryl group is substituted with one or more of groups selected from halo, C₁-C₈ alkyl, C₁-C₈ haloalkyl, cyano, hydroxy, C₁-C₈ alkoxy, and amino.

Examples of representative substituted aryls include the following

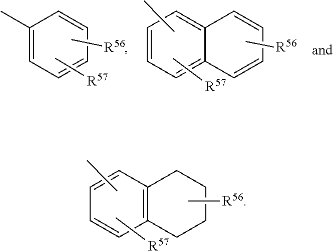

wherein one of R⁵⁶ and R⁵⁷ may be hydrogen and at least one of R⁵⁶ and R⁵⁷ is each independently selected from C₁-C₈ alkyl, C₁-C₈ haloalkyl, 4-10 membered heterocyclyl, alkanoyl, C₁-C₈ alkoxy, heteroaryloxy, alkylamino, arylamino, heteroarylamino, —NR⁵⁸COR⁵⁹, —NR⁵⁸SOR⁵⁹NR⁵⁸SO₂R⁵⁹, —COOalkyl, —COOaryl, —CONR⁵⁸R⁵⁹, —CONR⁵⁸OR⁵⁹, —NR⁵⁸R⁵⁹, —SO₂NR⁵⁸R⁵⁹, —S-alkyl, —SOalkyl, —SO₂alkyl, -Saryl, —SOaryl, —SO₂aryl; or R⁵⁶ and R⁵⁷ may be joined to form a cyclic ring (saturated or unsaturated) from 5 to 8 atoms, optionally containing one or more heteroatoms selected from the group consisting of N, O, or S. R⁶⁰ and R⁶¹ are independently hydrogen, —C₁-C₈ alkyl, —C₁-C₄haloalkyl, —C₃-C₁₀ cycloalkyl, 4-10 membered heterocyclyl, C₆-C₁₀ aryl, substituted C₆-C₁₀ aryl, 5-10 membered heteroaryl, or substituted 5-10 membered heteroaryl.

“Fused aryl” refers to an aryl having two of its ring carbons in common with a second aryl or heteroaryl ring or with a carbocyclyl or heterocyclyl ring.

“Heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, In such instances, unless otherwise specified, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In an embodiment, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In an embodiment, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In an embodiment, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In an embodiment, the 5-6 membered heteroaryl has 1-3 ring heteroatoms selected from nitrogen, oxygen, and sulfur. In an embodiment, the 5-6 membered heteroaryl has 1-2 ring heteroatoms selected from nitrogen, oxygen, and sulfur. In an embodiment, the 5-6 membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5-14 membered heteroaryl. In certain embodiments, the heteroaryl group is substituted 5-14 membered heteroaryl. In an embodiment, a heteroaryl group is a bicyclic 8-12 membered aromatic ring system having ring carbon atoms and 1-6 ring heteroatoms provided in the aromatic ring system, wherein each heteroatom is independently selected from nitrogen, oxygen, and sulfur (“8-12 membered bicyclic heteroaryl”). In an embodiment, a heteroaryl group is an 8-10 membered bicyclic aromatic ring system having ring carbon atoms and 1-6 ring heteroatoms provided in the aromatic ring system, wherein each heteroatom is independently selected from nitrogen, oxygen, and sulfur (“8-10 membered bicyclic heteroaryl”). In an embodiment, a heteroaryl group is a 9-10 membered bicyclic aromatic ring system having ring carbon atoms and 1-6 ring heteroatoms provided in the aromatic ring system, wherein each heteroatom is independently selected from nitrogen, oxygen, and sulfur (“9-10 membered bicyclic heteroaryl”). Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

Examples of representative heteroaryls include the following:

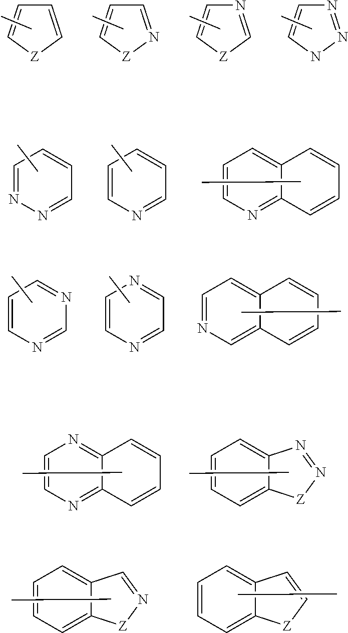

wherein each Z is selected from carbonyl, N, NR⁶⁵, O, and S; and R⁶⁵ is independently hydrogen, —C₁-C₈ alkyl, C₃-C₁₀ cycloalkyl, 4-10 membered heterocyclyl, C₆-C₁₀ aryl, and 5-10 membered heteroaryl.

In the structures described herein, a substituent attached to a polycyclic (e.g., bicyclic or tricyclic) cycloalkyl, heterocyclyl, aryl or heteroaryl with a bond that spans two or more rings is understood to mean that the substituent can be attached at any position in each of the rings.

“Heteroaralkyl” or “heteroarylalkyl” is a subset of “alkyl” and refers to an alkyl group substituted by a heteroaryl group, wherein the point of attachment is on the alkyl moiety.

The term “carbocyclyl” or “carbocyclic” refers to a radical of a non-aromatic monocyclic, bicyclic, or tricyclic or polycyclic hydrocarbon ring system having from 3 to 14 ring carbon atoms (“C₃₋₁₄ carbocyclyl”) and zero heteroatoms in the non-aromatic ring system. Carbocyclyl groups include fully saturated ring systems (e.g., cycloalkyls), and partially saturated ring systems. In an embodiment, a carbocyclyl group has 3 to 10 ring carbon atoms (“C₃₋₁₀ carbocyclyl”). In an embodiment, a carbocyclyl group has 3 to 8 ring carbon atoms (“C₃₋₈ carbocyclyl”). In an embodiment, a carbocyclyl group has 3 to 7 ring carbon atoms (“C₃₋₇ carbocyclyl”). In an embodiment, a carbocyclyl group has 3 to 6 ring carbon atoms (“C₃₋₆ carbocyclyl”). In an embodiment, a carbocyclyl group has 4 to 6 ring carbon atoms (“C₄₋₆ carbocyclyl”). In an embodiment, a carbocyclyl group has 5 to 6 ring carbon atoms (“C₅₋₆ carbocyclyl”). In an embodiment, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like.

As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

The term “cycloalkyl” as employed herein includes saturated cyclic, bicyclic, tricyclic, or polycyclic hydrocarbon groups having 3 to 14 carbons containing the indicated number of rings and carbon atoms (for example a C₃-C₁₋₄ monocyclic, C₄-C₁₋₄ bicyclic, C₅-C₁₄tricyclic, or C₆-C₁₋₄ polycyclic cycloalkyl). In an embodiment “cycloalkyl” is a monocyclic cycloalkyl. In an embodiment, a monocyclic cycloalkyl has 3-14 ring carbon atoms. (“C₃₋₁₄ monocyclic cycloalkyl”). In an embodiment, a monocyclic cycloalkyl group has 3 to 10 ring carbon atoms (“C₃₋₁₀ monocyclic cycloalkyl”). In an embodiment, a monocyclic cycloalkyl group has 3 to 8 ring carbon atoms (“C₃₋₈ monocyclic cycloalkyl”). In an embodiment, a monocyclic cycloalkyl group has 3 to 6 ring carbon atoms (“C₃₋₆ monocyclic cycloalkyl”). In an embodiment, a monocyclic cycloalkyl group has 4 to 6 ring carbon atoms (“C₄₋₆ monocyclic cycloalkyl”). In an embodiment, a monocyclic cycloalkyl group has 5 to 6 ring carbon atoms (“C₅₋₆ monocyclic cycloalkyl”). In an embodiment, a monocyclic cycloalkyl group has 5 to 10 ring carbon atoms (“C₅₋₁₀ monocyclic cycloalkyl”). Examples of monocyclic C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈).

In an embodiment “cycloalkyl” is a bicyclic cycloalkyl. In an embodiment, a bicyclic cycloalkyl has 4-14 ring carbon atoms. (“C₄₋₁₄ bicyclic cycloalkyl”). In an embodiment, a bicyclic cycloalkyl group has 4 to 12 ring carbon atoms (“C₄₋₁₂ bicyclic cycloalkyl”). In an embodiment, a bicyclic cycloalkyl group has 4 to 10 ring carbon atoms (“C₄₋₁₀ bicyclic cycloalkyl”). In an embodiment, a bicyclic cycloalkyl group has 5 to 10 ring carbon atoms (“C₅₋₁₀ bicyclic cycloalkyl”). In an embodiment, a bicyclic cycloalkyl group has 6 to 10 ring carbon atoms (“C₆₋₁₀ bicyclic cycloalkyl”). In an embodiment, a bicyclic cycloalkyl group has 8 to 10 ring carbon atoms (“C₅₋₁₀ bicyclic cycloalkyl”). In an embodiment, a bicyclic cycloalkyl group has 7 to 9 ring carbon atoms (“C₇₋₉ bicyclic cycloalkyl”). Examples of bicyclic cycloalkyls include bicyclo[1.1.0]butane (C₄), bicyclo[1.1.1]pentane (C₅), spiro[2.2]pentane (C₅), bicyclo[2.1.0]pentane (C₅), bicyclo[2.1.1]hexane (C₆), bicyclo[3.1.0]hexane (C₆), spiro[2.3]hexane (C₆), bicyclo[2.2.1]heptane (norbornane) (C₇), bicyclo[3.2.0]heptane (C₇), bicyclo[3.1.1]heptane (C₇), bicyclo[3.1.1]heptane (C₇), bicyclo[4.1.0]heptane (C₇), spiro[2.4]heptane (C₇), spiro [3.3]heptane (C₇), bicyclo[2.2.2]octane (C₈), bicyclo[4.1.1]octane (C₈)octahydropentalene (C₈), bicyclo[3.2.1]octane (C₈), bicyclo[4.2.0]octane (C₈), spiro[2.5]octane (C₈), spiro[3.4]octane (C₈), bicyclo[3.3.1]nonane (C₉), octahydro-1H-indene (C₉), bicyclo[4.2.1]nonane (C₉), spiro[3.5]nonane (C₉), spiro[4.4]nonane (C₉), bicyclo[3.3.2]decane (C₁₀), bicyclo[4.3.1]decane (C₁₀), spiro[4.5]decane (C₁₀), bicyclo[3.3.3]undecane (C₁₁), decahydronaphthalene (C₁₀), bicyclo[4.3.2]undecane (C₁₁), spiro[5.5]undecane (C₁₁) and bicyclo[4.3.3]dodecane (C₁₂).

In an embodiment “cycloalkyl” is a tricyclic cycloalkyl. In an embodiment, a tricyclic cycloalkyl has 6-14 ring carbon atoms. (“C₆₋₁₄ tricyclic cycloalkyl”). In an embodiment, a tricyclic cycloalkyl group has 8 to 12 ring carbon atoms (“C₈₋₁₂ tricyclic cycloalkyl”). In an embodiment, a tricyclic cycloalkyl group has 10 to 12 ring carbon atoms (“C₁₀₋₁₂ tricyclic cycloalkyl. Examples of tricyclic cycloalkyls include adamantine (C₁₂).

Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl.

“Heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In an embodiment, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In an embodiment, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In an embodiment, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In an embodiment, the 5-6 membered heterocyclyl has 1-3 ring heteroatoms selected from nitrogen, oxygen, and sulfur. In an embodiment, the 5-6 membered heterocyclyl has 1-2 ring heteroatoms selected from nitrogen, oxygen, and sulfur. In an embodiment, the 5-6 membered heterocyclyl has one ring heteroatom selected from nitrogen, oxygen, and sulfur.

Exemplary 3-membered heterocyclyl groups containing one heteroatom include, without limitation, aziridinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Nitrogen-containing heterocyclyl” group means a 4- to 7-membered non-aromatic cyclic group containing at least one nitrogen atom, for example, but without limitation, morpholine, piperidine (e.g., 2-piperidinyl, 3-piperidinyl and 4-piperidinyl), pyrrolidine (e.g., 2-pyrrolidinyl and 3-pyrrolidinyl), azetidine, pyrrolidone, imidazoline, imidazolidinone, 2-pyrazoline, pyrazolidine, piperazine, and N-alkyl piperazines such as N-methyl piperazine. Particular examples include azetidine, piperidone and piperazone.

“Hetero” when used to describe a compound or a group present on a compound means that one or more carbon atoms in the compound or group have been replaced by a nitrogen, oxygen, or sulfur heteroatom. Hetero may be applied to any of the hydrocarbyl groups described above such as alkyl, e.g., heteroalkyl, cycloalkyl, e.g., heterocyclyl, aryl, e.g., heteroaryl, cycloalkenyl, e.g., cycloheteroalkenyl, and the like having from 1 to 5, and particularly from 1 to 3 heteroatoms.

“Acyl” refers to a radical —C(═O)R²⁰, where R²⁰ is hydrogen, substituted or unsubstitued alkyl, substituted or unsubstitued alkenyl, substituted or unsubstitued alkynyl, substituted or unsubstitued carbocyclyl, substituted or unsubstituted heterocyclyl, substituted or unsubstituted aryl, or substituted or unsubstitued heteroaryl, as defined herein. “Alkanoyl” is an acyl group wherein R²⁰ is a group other than hydrogen. Representative acyl groups include, but are not limited to, formyl (—CHO), acetyl (—C(═O)CH₃), cyclohexylcarbonyl, cyclohexylmethylcarbonyl, benzoyl (—C(═O)Ph), benzylcarbonyl (—C(═O)CH₂Ph), —C(═O)—C₁-C₈ alkyl, —C(═O)—(CH₂)ₜ(C₆-C₁₀ aryl), —C(═O)—(CH₂)ₜ(5-10 membered heteroaryl), —C(═O)—(CH₂)ₜ(C₃-C₁₀ cycloalkyl), and —C(═O)—(CH₂)ₜ(4-10 membered heterocyclyl), wherein t is an integer from 0 to 4. In certain embodiments, R²¹ is C₁-C₈ alkyl, substituted with halo or hydroxy; or C₃-C₁₀ cycloalkyl, 4-10 membered heterocyclyl, C₆-C₁₀ aryl, arylalkyl, 5-10 membered heteroaryl or heteroarylalkyl, each of which is substituted with unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

The term aminoalkyl refers to a substituted alkyl group wherein one or more of the hydrogen atoms are independently replaced by an —NH₂ group.

The term hydroxyalkyl refers to a substituted alkyl group wherein one or more of the hydrogen atoms are independently replaced by an —OH group.

The terms “alkylamino” and “dialkylamino” refer to —NH(alkyl) and —N(alkyl)₂ radicals respectively. In an embodiment the alkylamino is a —NH(C₁-C₄ alkyl). In an embodiment the alkylamino is methylamino, ethylamino, propylamino, isopropylamino, n-butylamino, iso-butylamino, sec-butylamino or tert-butylamino. In an embodiment the dialkylamino is —N(C₁-C₆ alkyl)₂. In an embodiment the dialkylamino is a dimethylamino, a methylethylamino, a diethylamino, a methylpropylamino, a methylisopropylamino, a methylbutylamino, a methylisobutylamino or a methyltertbutylamino.

The term “aryloxy” refers to an —O-aryl radical. In an embodiment the aryloxy group is phenoxy.

The term “haloalkoxy” refers to alkoxy structures that are substituted with one or more halo groups or with combinations thereof. For example, the term “fluoroalkoxy” includes haloalkoxy groups, in which the halo is fluorine. In an embodiment haloalkoxy groups are difluoromethoxy and trifluoromethoxy.

“Alkoxy” refers to the group —OR²⁹ where R²⁹ is substituted or unsubstituted alkyl, substituted or unsubstitued alkenyl, substituted or unsubstitued alkynyl, substituted or unsubstitued carbocyclyl, substituted or unsubstituted heterocyclyl, substituted or unsubstituted aryl, or substituted or unsubstitued heteroaryl. Particular alkoxy groups are methoxy, ethoxy, n-propoxy, isopropoxy, n-butoxy, tert-butoxy, sec-butoxy, n-pentoxy, n-hexoxy, and 1,2-dimethylbutoxy. Particular alkoxy groups are lower alkoxy, i.e. with between 1 and 6 carbon atoms. Further particular alkoxy groups have between 1 and 4 carbon atoms.

In certain embodiments, R²⁹ is a group that has 1 or more substituents, for instance from 1 to 5 substituents, and particularly from 1 to 3 substituents, in particular 1 substituent, selected from the group consisting of amino, substituted amino, C₆-C₁₀ aryl, aryloxy, carboxyl, cyano, C₃-C₁₀ cycloalkyl, 4-10 membered heterocyclyl, halogen, 5-10 membered heteroaryl, hydroxyl, nitro, thioalkoxy, thioaryloxy, thiol, alkyl-S(O)—, aryl-S(O)—, alkyl-S(O)₂— and aryl-S(O)₂—. Exemplary ‘substituted alkoxy’ groups include, but are not limited to, —O—(CH₂)ₜ(C₆-C₁₀ aryl), —O—(CH₂)ₜ(5-10 membered heteroaryl), —O—(CH₂)ₜ(C₃-C₁₀ cycloalkyl), and —O—(CH₂)ₜ(4-10 membered heterocyclyl), wherein t is an integer from 0 to 4 and any aryl, heteroaryl, cycloalkyl or heterocyclyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. Particular exemplary ‘substituted alkoxy’ groups are —OCF₃, —OCH₂CF₃, —OCH₂Ph, —OCH₂-cyclopropyl, —OCH₂CH₂OH, and —OCH₂CH₂N(CH₃)₂.

“Amino” refers to the radical —NH₂.

“Oxo group” refers to —C(═O)—.

“Substituted amino” refers to an amino group of the formula —N(R³⁸)₂ wherein R³⁸ is hydrogen, substituted or unsubstituted alkyl, substituted or unsubstitued alkenyl, substituted or unsubstitued alkynyl, substituted or unsubstitued carbocyclyl, substituted or unsubstituted heterocyclyl, substituted or unsubstituted aryl, substituted or unsubstitued heteroaryl, or an amino protecting group, wherein at least one of R³⁸ is not a hydrogen. In certain embodiments, each R³⁸ is independently selected from hydrogen, —C₁-C₈ alkyl, —C₃-C₈ alkenyl, —C₃-C₈ alkynyl, C₆-C₁₀ aryl, 5-10 membered heteroaryl, 4-10 membered heterocyclyl, or C₃-C₁₀ cycloalkyl; or C₁-C₅ alkyl, substituted with halo or hydroxy; C₃-C₈ alkenyl, substituted with halo or hydroxy; C₃-C₅ alkynyl, substituted with halo or hydroxy, or —(CH₂)ₜ(C₆-C₁₀ aryl), —(CH₂)ₜ(5-10 membered heteroaryl), —(CH₂)ₜ(C₃-C₁₀ cycloalkyl), or —(CH₂)ₜ(4-10 membered heterocyclyl), wherein t is an integer between 0 and 8, each of which is substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy; or both R³⁸ groups are joined to form an alkylene group.

Exemplary “substituted amino” groups include, but are not limited to, —NR³⁹—C₁-C₅ alkyl, —NR³⁹—(CH₂)ₜ(C₆-C₁o aryl), —NR³⁹—(CH₂)ₜ(5-10 membered heteroaryl), —NR³⁹—(CH₂)ₜ(C₃-C₁₀ cycloalkyl), and —NR³⁹—(CH₂)ₜ(4-10 membered heterocyclyl), wherein t is an integer from 0 to 4, for instance 1 or 2, each R³⁹ independently represents H or C₁-C₈ alkyl; and any alkyl groups present, may themselves be substituted by halo, substituted or unsubstituted amino, or hydroxy; and any aryl, heteroaryl, cycloalkyl, or heterocyclyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. For the avoidance of doubt the term ‘substituted amino’ includes the groups alkylamino, substituted alkylamino, alkylarylamino, substituted alkylarylamino, arylamino, substituted arylamino, dialkylamino, and substituted dialkylamino as defined below. Substituted amino encompasses both monosubstituted amino and disubstituted amino groups.

In certain embodiments, the substituent present on the nitrogen atom is a nitrogen protecting group (also referred to herein as an “amino protecting group”). Nitrogen protecting groups include, but are not limited to, —OH, —OR^{aa}, —N(R^{cc})₂, —C(═O)R^{aa}, —C(═O)N(R^{cc})₂—CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, C(═S)N(R^{cc})₂, —C(═O)SR^{cc}, —C(═S)SR^{cc}, —C₁₋₁₀ alkyl (e.g., aralkyl, heteroaralkyl), —C₂₋₁₀ alkenyl, —C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.
each instance of R^{aa} is, independently, selected from —C₁₋₁₀ alkyl, —C₁₋₁₀ perhaloalkyl, —C₂₋₁₀ alkenyl, —C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}—N(R^{cc})₂, —CN, —C(═O)R^{aa}, —C(═O)N(R^{cc})₂, —CO₂R^{aa}, —SO₂R^{aa}, C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}—C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, —C₁₋₁₀ alkyl, —C₁₋₁₀ perhaloalkyl, —C₂₋₁₀ alkenyl, —C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X is a counterion.
each instance of R^{cc} is, independently, selected from hydrogen, —C₁₋₁₀ alkyl, —C₁₋₁₀ perhaloalkyl, —C₂₋₁₀ alkenyl, —C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{cc})₃⁺X, —N(OR^{ee})R^{ff}, —SH, —SR^{ee}, —SSR^{ee}, —C(═O)R^{ee}, —CO₂H, —CO₂R^{cc}, —OC(═O)R^{ee}, —OCO₂R^{cc}, —C(═O)N(R^{ff})₂, —OC(═O)N(R^{ff})₂, —NR^{ff}C(═O)R^{ee}, —NR^{ee}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ee}C(═NR^{ff})N(R^{ff})₂, —NR^{ee}SO₂R^{cc}, —SO₂N(R^{ff})₂, —SO₂R^{cc}, —SO₂OR^{cc}, —OSO₂R^{cc}, —S(═O)R^{cc}, —Si(R^{cc})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{cc})₂, —OP(═O)(OR^{ee})₂, —C₁₋₆ alkyl, —C₁₋₆ perhaloalkyl, —C₂₋₆ alkenyl, —C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;
each instance of R^{ee} is, independently, selected from —C₁₋₆ alkyl, —C₁₋₆ perhaloalkyl, —C₂₋₆ alkenyl, —C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{f} is, independently, selected from hydrogen, —C₁₋₆ alkyl, —C₁₋₆ perhaloalkyl, —C₂₋₆ alkenyl, —C₂₋₆ alkynyl, heteroC₁₋₆alkyl, heteroC₂₋₆alkenyl, heteroC₂₋₆ alkynyl, C₃₋₁₀ carbocyclyl, 3-10 membered heterocyclyl, C₆₋₁₀ aryl and 5-10 membered heteroaryl, or two R^{f} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)+X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃—C(═S)N(C₁₋₆ alkyl)₂, —C(═S)NH(C₁₋₆ alkyl), —C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, —C₁₋₆ alkyl, —C₁₋₆ perhaloalkyl, —C₂₋₆ alkenyl, —C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, 5-10 membered heteroaryl; or two geminal R⁹⁹ substituents can be joined to form ═O or ═S; wherein X⁻ is a counterion.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitrophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluorenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), 15 m-nitrophenyl carbamate, 3,5-dimethoxybenzyl carbamate, o-nitrobenzyl carbamate, 3,4-dimethoxy-6-nitrobenzyl carbamate, phenyl (o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-10doethyl carbamate, isobornyl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), (β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to herein as an “hydroxyl protecting group”). Oxygen protecting groups include, but are not limited to, —R^{aa}, N(R^{bb})₂, —C(═O)SR^{aa}, —C(═O)R^{aa}—CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃X⁻, —P(OR^{cc})₂, —P(OR^{cc})₃X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxymethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-l′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-10dobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, u-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is a sulfur protecting group (also referred to as a “thiol protecting group”). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃X, —P(OR^{cc})₂—P(OR^{cc})₃X, —P(═O)(R^{aa})₂, —P(═O)(OR^{cc})₂ and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

The term “leaving group” is given its ordinary meaning in the art of synthetic organic chemistry and refers to an atom or a group capable of being displaced by a nucleophile. Examples of suitable leaving groups include, but are not limited to, halogen (such as F, —Cl, —Br, or I (iodine)), alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In certain embodiments, the leaving group is halogen, alkanesulfonyloxy, arenesulfonyloxy, diazonium, alkyl diazenes, aryl diazenes, alkyl triazenes, aryl triazenes, nitro, alkyl nitrate, aryl nitrate, alkyl phosphate, aryl phosphate, alkyl carbonyl oxy, aryl carbonyl oxy, alkoxcarbonyl oxy, aryoxcarbonyl oxy ammonia, alkyl amines, aryl amines, hydroxyl group, alkyloxy group, or aryloxy. In some cases, the leaving group is a sulfonic acid ester, such as toluenesulfonate (tosylate, -OTs), methanesulfonate (mesylate, -OMs), p-bromobenzenesulfonyloxy (brosylate, -OBs), —OS(═O)₂(CF₂)₃CF₃ (nonaflate, —ONf), or trifluoromethanesulfonate (triflate, -OTf). In some cases, the leaving group is a brosylate, such as p-bromobenzenesulfonyloxy. In some cases, the leaving group is a nosylate, such as 2-nitrobenzenesulfonyloxy. In an embodiment, the leaving group is a sulfonate-containing group. In an embodiment, the leaving group is a tosylate group. The leaving group may also be a phosphineoxide (e.g., formed during a Mitsunobu reaction) or an internal leaving group such as an epoxide or cyclic sulfate. Other non-limiting examples of leaving groups are water, ammonia, alcohols, ether moieties, thioether moieties, zinc halides, magnesium moieties, diazonium salts, and copper moieties.

“Carboxy” refers to the radical —C(═O)OH.

“Cyano” refers to the radical —CN.

“Halo” or “halogen” refers to fluoro (F), chloro (Cl), bromo (Br), and iodo (I). In certain embodiments, the halo group is either fluoro or chloro.

“Haloalkyl” refers to an alkyl radical in which the alkyl group is substituted with one or more halogens. Typical haloalkyl groups include, but are not limited to, trifluoromethyl (—CF₃), difluoromethyl (—CHF₂), fluoromethyl (—CH₂F), chloromethyl (—CH₂Cl), dichloromethyl (—CHCl₂), tribromomethyl (—CH₂Br), and the like.

“Hydroxy” refers to the radical —OH.

“Nitro” refers to the radical —NO₂.

“Thioketo” refers to the group ═S.

Alkyl, alkenyl, alkynyl, carbocyclyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. Any and all such combinations are contemplated in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃X, —N(OR^{cc})Rb, —SH, —SR^{aa}, —SSR^{cc}, —C(═O)R^{aa}, —CO₂H, —CHO, —C(OR^{cc})₂, —CO₂R^{aa}, —OC(═O)R^{aa}, OCO₂R^{aa}, —C(═O)N(R^{bb})₂, —OC(═O)N(R^{bb})₂, —NR^{bb}C(═O)R^{aa}, —NR^{bb}CO₂R^{aa}, NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —S(═O)(═NR^{bb})R^{aa}, OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), —C₁₋₁₀ alkyl, —C₁₋₁₀ haloalkyl, —C₂₋₁₀ alkenyl, —C₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups; or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNRbbS(═O)₂R^{aa}, ═NR^{bb}, or ═NOR^{cc}; each instance of R^{aa} is, independently, selected from C₁₋₁₀ alkyl, —C₁₋₁₀ haloalkyl, —C₂₋₁₀ alkenyl, —C₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, N(R^{cc})₂, —CN, —C(═O)R^{aa}, —C(═O)N(R^{ee})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{ee})₂, —P(═O)(NR^{ee})₂, —C₁₋₁₀ alkyl, —C₁₋₁₀ haloalkyl, —C₂₋₁₀ alkenyl, —C₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{cc} is, independently, selected from hydrogen, —C₁₋₁₀ alkyl, —C₁₋₁₀ haloalkyl, —C₂₋₁₀ alkenyl, —C₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{cc})₃⁺X⁻, —N(OR^{ee})R^{ff}, —SH, —SR^{ee}, —SSR^{ee}, —C(═O)R^{ee}, —CO₂H, —CO₂R^{cc}, —OC(═O)R^{ee}, —OCO₂R^{ee}, —C(═O)N(R^{ff})₂, —OC(═O)N(R^{ff})₂, —NR^{ee}C(═O)R^{ee}, —NR^{ff}CO₂R^{ee}, —NR^{ee}C(═O)N(R^{ff})₂, —C(═NR^{ff})OR^{ee}, —OC(═NR^{ff})R^{ee}, —OC(═NR^{ee})OR^{ee}, —C(═NR^{ff})N(R^{ff})₂, —OC(═NR^{ff})N(R^{ff})₂, —NR^{ff}C(═NR^{ff})N(R^{ff})₂, —NR^{ee}SO₂R^{ee}, —SO₂N(R^{ff})₂, —SO₂R^{ee}, —SO₂OR^{cc}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, —C₁₋₆ alkyl, —C₁₋₆ haloalkyl, —C₂₋₆ alkenyl, —C₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;
each instance of R^{cc} is, independently, selected from C₁₋₆ alkyl, —C₁₋₆ haloalkyl, —C₂₋₆ alkenyl, —C₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups;
each instance of R^{f} is, independently, selected from hydrogen, —C₁₋₆ alkyl, —C₁₋₆ haloalkyl, —C₂₋₆ alkenyl, —C₂₋₆ alkynyl, C₃₋₁₀ carbocyclyl, 3-10 membered heterocyclyl, C₆₋₁₀ aryl and 5-10 membered heteroaryl, or two R^{f} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X, —NH(C₁₋₆ alkyl)₂⁺X, —NH₂(C₁₋₆ alkyl)+X, —NH₃⁺X, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃—C(═S)N(C₁₋₆ alkyl)₂, —C(═S)NH(C₁₋₆ alkyl), —C(═S)NH₂, —C(═O)S(C₁₋₆ alkyl), —C(═S)SC₁₋₆alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ alkyl)₂, —C₁₋₆ alkyl, —C₁₋₆ haloalkyl, —C₂₋₆ alkenyl, —C₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X is a counterion.

A “counterion” or “anionic counterion” is a negatively charged group associated with a cationic quatemary amino group in order to maintain electronic neutrality. Exemplary counterions include halide ions (e.g., F⁻, Cl⁻, Br⁻, I⁻), NO₃⁻, ClO₄⁻, OH⁻, H₂PO₄⁻, HSO₄⁻, SO₄⁻² sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

Nitrogen atoms can be substituted or unsubstituted as valency permits, and include primary, secondary, tertiary, and quarternary nitrogen atoms. Exemplary nitrogen atom substitutents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, —C₁₋₁₀ alkyl, —C₁₋₁₀ haloalkyl, —C₂₋₁₀ alkenyl, —C₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

These and other exemplary substituents are described in more detail in the Detailed Description, Examples, and Claims. The invention is not intended to be limited in any manner by the above exemplary listing of substituents.

### Other Definitions

As used herein, “pharmaceutical composition” or “pharmaceutical formulation” refer to the combination of a therapeutically active agent with a pharmaceutically acceptable excipient, inert or active, making the composition especially suitable for diagnostic or therapeutic use in vivo or ex vivo.

“Pharmaceutically acceptable” refers to compounds, molecular entities, compositions, materials and/or dosage forms that do not produce an adverse, allergic or other untoward reaction when administered to an animal, or human, as appropriate; or means approved or approvable by a regulatory agency of the federal or a state government or the corresponding agency in countries other than the United States, or that is listed in the U.S. Pharmacopoeia or other generally recognized pharmacopoeia for use in animals, and more particularly, in humans.

As used herein, “pharmaceutically acceptable salt” refers to any salt of an acidic or a basic group that may be present in a compound of the present disclosure (e.g., the compound of Formula A-I or formula I), which salt is compatible with pharmaceutical administration.

As is known to those of skill in the art, “salts” of compounds may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic and benzenesulfonic acid. Other acids, such as oxalic, while not in themselves pharmaceutically acceptable, may be employed in the preparation of salts useful as intermediates in obtaining the compounds described herein and their pharmaceutically acceptable acid addition salts.

Examples of bases include, but are not limited to, alkali metal (e.g., sodium and potassium) hydroxides, alkaline earth metal (e.g., magnesium and calcium) hydroxides, ammonia, and compounds of formula NW₄⁺, wherein W is C1-4 alkyl, and the like.

Examples of salts include, but are not limited, to acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present disclosure compounded with a suitable cation such as Na+, K+, Ca2⁺, NH4+, and NW4+ (where W can be a C1-4 alkyl group), and the like.

For therapeutic use, salts of the compounds of the present disclosure are contemplated as being pharmaceutically acceptable. However, salts of acids and bases that are non-pharmaceutically acceptable may also find use, for example, in the preparation or purification of a pharmaceutically acceptable compound.

As used herein, “pharmaceutically acceptable excipient” refers to a substance that aids the administration of an active agent to and/or absorption by a subject and can be included in the compositions of the present disclosure without causing a significant adverse toxicological effect on the patient. Non-limiting examples of pharmaceutically acceptable excipients include binders, diluents, carriers, adjuvants, fillers (e.g., brittle diluents or fillers and ductile diluents or fillers), disintegrants, lubricants, coatings, sweeteners, flavors, gelatins, carbohydrates such as lactose, amylose or starch, fatty acid esters, hydroxypropylmethylcellulose, polyvinyl pyrrolidine, and colors, and the like. For examples of excipients, see Gennaro, Remington's Pharmaceutical Sciences, 18th Ed., Mack Publ. Co., Easton, PA (1990) or Shesky, Hancock, Moss and Goldfarb, Handbook of Pharmaceutical Excipients, 9th Ed. Pharmaceutical Press, London, UK (2020).

Examples of diluents or fillers include, but are not limited to, a sugar (e.g., mannitol, lactose, sorbitol, lactitol, erythritol, sucrose, fructose, glucose, agarose, maltose, isomalt, polydextrose, and combinations thereof), an inorganic material (e.g., dibasic calcium phosphate, hydroxyapatite, sodium carbonate, sodium bicarbonate, calcium carbonate, calcium sulfate, magnesium carbonate, magnesium oxide, bentonite, kaolin), calcium lactate, a starch (e.g., a pregelatinized starch), a microcrystalline cellulose, a silicified microcrystalline cellulose, a polysaccharide, a cellulose (e.g., a hydroxypropylcellulose, a hypromellose, a carboxymethylcellulose, a methylcellulose, a hydroxypropylmethylcellulose, a hydroxyethylcellulose), a dextrin, a maltodextrin, an alginate, a collagen, a polyvinylpyrrolidone, a polyvinylacrylate, polyethylene oxide, and polyethylene glycol. Sugar is defined herein to include sugar alcohols.

Examples of disintegrants include, but are not limited to, alginic acid, an alginate, primogel, a cellulose (e.g., hydroxypropylcellulose), polacrillin potassium, sodium starch glycolate, sodium croscarmellose, a polyplasdone (e.g., a crospovidone), and a starch (e.g., corn starch, pregelatinized starch, hydroxypropyl starch, and carboxymethyl starch).

Examples of binders include, but are not limited to, a hydroxypropylcellulose, hydroxyethylcellulose, a hydroxypropylmethycellulose (e.g., a low viscosity hydroxypropylmethycellulose), a sugar, a polyvinylpyrrolidone, a polyvinyl alcohol, a polyvinyl acetate, a polydextrose, a chitosan, a carrageenan, carbophil, a microcrystalline cellulose, gum tragacanth, guar gum, gellan gum, gelatin, and a starch (e.g., corn starch).

Examples of wetting agents include, but are not limited to, a poloxamer (e.g., poloxamer 407), sodium dodecyl sulfate, sodium lauryl sulfate (SLS), sodium stearyl fumarate (SSF), a polydimethylsiloxane, a polysorbate (e.g., polyoxyethylene 20 sorbitan mono-oleate (Tween® 20)), sorbitan monooleate, sorbitan trioleate, sorbitan laurate, sorbitan stearate, sorbitan monopalmitate, lecithin, sodium taurocholate, ursodeoxycholate, polyethoxylated castor oil, cetyl trimethylammonium bromide, nonoxynol, 6-tocopherol polyethylene glycol 1000 succinate, and docusate sodium.

Examples of lubricants and glidants include, but are not limited to, a wax, a glyceride, a light mineral oil, a polyethylene glycol, sodium stearyl fumarate, magnesium stearate, stearic acid, hydrogenated oil (e.g., hydrogenated vegetable oil), an alkyl sulfate, sodium benzoate, sodium acetate, glyceryl behenate, palmitic acid, and coconut oil.

Examples of glidants include, but are not limited to, colloidal silicon dioxide, colloidal silicon dioxide, talc, kaolin, bentonite, and activated carbon/charcoal.

Examples of colorants include, but are not limited to, titanium dioxide, aluminum lakes, iron oxides and carbon black.

Examples of coatings include but are not limited to, a film forming polymer (e.g., a hypromellose, a methyl cellulose, an ethylcellulose, cellulose acetate, a hydroxypropylmethyl cellulose, a hydroxypropyl cellulose, hydroxypropylmethyl cellulose acetate succinate, cellulose acetate phthalate, a polyvinylpyrrolidone, polyvinyl alcohol, a Eudragit/acrylate) and a plasticizer (e.g., triacetin, polyethylene glycol, propylene glycol).

Pharmaceutical compositions for oral administration (e.g., pharmaceutical compositions of the compound of Formula A-I or formula I described herein) can take the form of bulk liquid solutions or suspensions or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include pills, tablets, capsules or the like in the case of solid compositions.

A “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal.

As used herein, “solid dosage form” means a pharmaceutical dose(s) in solid form, e.g., tablets, capsules, granules, powders, minitabs, sachets, stickpacks, reconstitutable powders, dry powder inhalers, lozenges, and chewables.

As used herein, “administering” means oral administration, administration as a pulmonary, suppository, intramuscular administration, intrathecal administration, intranasal administration or subcutaneous administration, or the implantation of a slow-release device, e.g., a mini-osmotic pump, to a subject. Administration is by any route, including transmucosal (e.g., buccal, sublingual, palatal, gingival, nasal, vaginal, rectal, or). Parenteral administration includes, e.g., intramuscular and subcutaneous. Other modes of delivery include, but are not limited to, the use of liposomal formulations, etc. By “co-administer” it is meant that a composition described herein is administered at the same time, just prior to, or just after the administration of one or more additional therapies (e.g., anti-cancer agent, chemotherapeutic, or treatment for a neurodegenerative disease). The compound of Formula A-I or formula I can be administered alone or can be co-administered to the patient. Co-administration is meant to include simultaneous or sequential administration of the compound individually or in combination (more than one compound or agent). Thus, the preparations can also be combined, when desired, with other active substances (e.g., to reduce metabolic degradation).

The terms “disease,” “disorder,” and “condition” are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (“therapeutic treatment”), and also contemplates an action that occurs before a subject begins to suffer from the specified disease, disorder or condition (“prophylactic treatment”). In an embodiment, the compounds provided herein are contemplated to be used in methods of therapeutic treatment wherein the action occurs while a subject is suffering from the specified disease, disorder or condition and results in a reduction in the severity of the disease, disorder or condition, or retardation or slowing of the progression of the disease, disorder or condition. In an alternate embodiment, the compounds provided herein are contemplated to be used in methods of prophylactic treatment wherein the action occurs before a subject begins to suffer from the specified disease, disorder or condition and results in preventing a disease, disorder or condition, or one or more symptoms associated with the disease, disorder or condition, or preventing the recurrence of the disease, disorder or condition.

In general, the “effective amount” of a compound refers to an amount sufficient to elicit the desired biological response e.g., to treat a disease or disorder described herein. As will be appreciated by those of ordinary skill in this art, the effective amount of a compound of the disclosure may vary depending on such factors as the desired biological endpoint, the pharmacokinetics of the compound, the disease being treated, the mode of administration, and the age, health, and condition of the subject. An effective amount encompasses therapeutic and prophylactic treatment (i.e., encompasses a “therapeutically effective amount” and a “prophylactically effective amount”).

As used herein, and unless otherwise specified, a “therapeutically effective amount” of a compound is an amount sufficient to provide a therapeutic benefit in the therapeutic treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the therapeutic treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

As used herein, and unless otherwise specified, a “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent.

As used herein, the term “selective” refers to a compound that is at least about 3-fold more potent (e.g., 3-fold, 5-fold, 10-fold, 20-fold, 30-fold, 50-fold, 100-fold, 500-fold, 1000 fold) against one target compared to other targets. For example, a CDK2 degrader that is selective over CCNE (CCNE1 and/or CCNE2) is at least 3-fold more potent (e.g., 3-fold, 5-fold, 10-fold, 20-fold, 30-fold, 50-fold, 100-fold, 500-fold, 1000 fold) more potent against CDK2 than against CCNE (CCNE1 and/or CCNE2). For example, a CCNE (CCNE1 and/or CCNE2) degrader that is selective over CDK2 is at least 3-fold potent (e.g., 3-fold, 5-fold, 10-fold, 20-fold, 30-fold, 50-fold, 100-fold, 500-fold, 1000 fold) more potent against CCNE (CCNE1 and/or CCNE2) than against CDK2. The difference in potency can be determined, for example, by comparing the DC₅₀ values against different targets.

### Compounds

Provided herein are compounds of Formula A-I and Formula I. Unless the context requires otherwise, reference throughout this specification to “a compound of Formula A-I or Formula I” or “compounds of Formula A-I or Formula I” refers to all embodiments of Formula A-I and Formula I, including, for example, compounds of Formula A-I, A-I-A, A-I-B, A-I-1, A-I-2, A-I-A1, A-I-A2, A-I-1, A-I-B2, A-II, A-II-A, A-II-B, A-II-1, A-II-2, A-II-A1, A-II-A2, A-II-1, A-II-B2, A-III, A-III-A, A-III-B, A-III-1, A-III-2, A-III-A1, A-III-A2, A-III-B1, A-III-B2, A-IV, A-IV-A, A-IV-B, A-IV-1, A-IV-2, A-IV-AI, A-IV-A2, A-IV-B1, A-IV-B2, A-V, A-V-A, A-V-B, A-V-1, A-V-2, A-V-A1, A-V-A2, A-V-B1, A-V-B2, A-VI, A-VI-A, A-VI-B, A-VI-1, A-VI-2, A-VI-A1, A-VI-A2, A-VI-B1, A-VI-B2, A-VII, A-VII-A, A-VII-B, A-VII-1, A-VII-2, A-VII-A1, A-VII-A2, A-VII-B1, A-VII-B2, A-VIII, A-VIII-A, A-VIII-B, A-VIII-1, A-VIII-2, A-VIII-A1, A-VIII-A2, A-VIII-B1, A-VIII-B2, I-1, I-2, II, II-1, II-2, II-a, II-a-1, II-a-2, II-b, II-b-1, II-b-2, III, III-1, III-2, III-a, III-a-1, III-a-2, III-b, III-b-1, III-b-2, III-c, III-c-1, III-c-2, IV, IV-a, IV-b, IV-c, IV-d, IV-e, IV-f, IV-g and IV-i as well as the compounds of Table 1. In an embodiment, provided are compounds of Formula A-I and Formula I or pharmaceutically acceptable salts thereof. In an embodiment, the compounds of Formula A-I and Formula I are provided as pharmaceutically acceptable salts. In an embodiment, the compounds of Formula A-I and Formula I are provided as the corresponding free base (i.e., are not salts).

Included herein, when chemically relevant, are all stereoisomers of the compounds, including diastereomers and enantiomers. Also included are mixtures of possible stereoisomers in any ratio, including, but not limited to, racemic mixtures. Unless stereochemistry is explicitly indicated in a structure, the structure is intended to embrace all possible stereoisomers of the compound depicted. If stereochemistry is explicitly indicated for one portion or portions of a molecule, but not for another portion or portions of a molecule, the structure is intended to embrace all possible stereoisomers for the portion or portions where stereochemistry is not explicitly indicated.

In one aspect, provided is a compound of Formula A-I

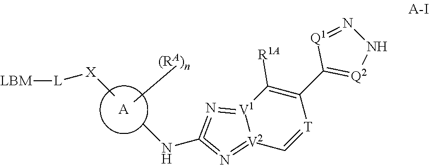

or a pharmaceutically acceptable salt thereof, wherein:
[symbol] is a single or a double bond;
Ring A is selected from the group consisting of a nitrogen-containing 4-10 member heterocyclyl, a C₆-o aryl and a 5-10-member heteroaryl, wherein the 4-10 member heterocyclyl, C₆₋₁₀ aryl and 5-10-member heteroaryl are attached to the —NH— through a carbon atom;
V¹ is nitrogen and V² is carbon, and Ring

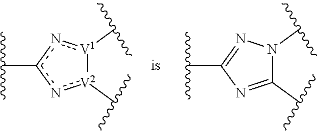

or V² is nitrogen and V¹ is carbon, and Ring

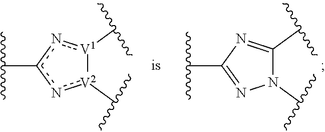

T is CH or N;
Q¹ and Q² are independently selected from N and CH;
R^{1A} is independently selected from H, D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocyclyl, 5-10 membered heteroaryl, C₃₋₁₀ cycloalkyl-C₁₋₄ alkyl-, 6-10 membered aryl-C₁₋₄ alkyl-, 4-10 membered heterocyclyl-C₁₋₄ alkyl-, 5-10 membered heteroaryl-C₁₋₄ alkyl-, OR^{a1}, SR^{a1} NHOR^{a1}, C(O)R^{b1}, C(O)NR^{a1}R^{a1}, C(O)NR^{a1}(OR^{a1}), C(O)OR^{a1}, OC(O)R^{b1}, OC(O)NR^{a1}R^{a1}, NR^{a1}R^{a1}, NR^{a1}NR^{a1}R^{a1}, NR^{a1}C(O)R^{b1}, NR^{a1}C(O)OR^{a1}, NR^{a1}C(O)NR^{a1}R^{a1}, C(═NR^{a1})R^{b1}, C(═NR^{a1})NR^{a1}R^{a1}, NR^{a1}C(═NR^{a1})NR^{a1}R^{a1}, NR^{a1}C(═NR^{a1})R^{b1}, NR^{a1}S(O)NR^{a1}R^{a1}, NR^{a1}S(O)R^{a1}, NR^{a1}S(O)₂R^{b1}, NR^{a1}S(O)(═NR^{a1})R^{b1}, NR^{a1}S(O)₂NR^{a1}R^{a1}, S(O)R^{b1}, S(O)NR^{a1}R^{a1}, S(O)₂R^{b1}, S(O)₂NR^{a1}R^{a1}, OS(O)(═NR^{a1})R^{b1}, OS(O)₂R^{b1}, S(O)(═NR^{a1})R^{b1}, SF₅, P(O)R^{a1}R^{b1}, OP(O)(OR^{a1})(OR^{a1}) and P(O)(OR^{a1})(OR^{a1}), wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocyclyl, 5-10 membered heteroaryl, C₃₋₁₀ cycloalkyl-C₁₋₄ alkyl-, 6-10 membered aryl-C₁₋₄ alkyl-, 4-10 membered heterocyclyl-C₁₋₄ alkyl-, and 5-10 membered heteroaryl-C₁₋₄ alkyl- are each substituted with 0, 1, 2, 3, or 4 independently selected R² substituents;
each R² is independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocyclyl, 5-6 membered heteroaryl, C₃₋₇ cycloalkyl-C₁₋₄ alkyl-, phenyl-C₁₋₄ alkyl-, 4-7 membered heterocyclyl-C₁₋₄ alkyl-, 5-6 membered heteroaryl-C₁₋₄ alkyl-, OR^{a2}, SR^{a2}, NHOR^{a2}, C(O)R^{b2}, C(O)NR^{a2}R^{a2}, C(O)NR^{a2}(OR^{a2}), C(O)OR^{a2}, OC(O)R^{b2}, OC(O)NR^{a2}R^{a2}, NR^{a2}R^{a2}, NR^{a2}NR^{a2}R^{a2}, NR^{a2}C(O)R^{b2}, NR^{a2}C(O)OR^{a2}, NR^{a2}C(O)NR^{a2}R^{a2}, C(═NR^{a2})R^{b2}, C(═NR^{a2})NR^{a2}R^{a2}, NR^{a2}C(═NR^{a2})NR^{a2}R^{a2}, NR^{a2}C(═NR^{a2})R^{b2}, NR^{a2}S(O)NR^{a2}R^{a2}, NR^{a2}S(O)R^{b2}, NR^{a2}S(O)₂R^{b2}, NR^{a2}S(O)(═NR^{a2})R^{b2}, NR^{a2}S(O)₂NR^{a2}R^{a2}, S(O)R^{b2}, S(O)NR^{a2}R^{a2}, S(O)₂R^{b2}, S(O)₂NR^{a2}R^{a2}, OS(O)(═NR^{a2})R^{b2}, OS(O)₂R^{b2}, S(O)(═NR^{a2})R^{b2}, SF₅, P(O)R^{a2}R^{a2}, OP(O)(OR^{a2})(OR^{a2}) and P(O)(OR^{a2})(OR^{a2}), wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocyclyl, 5-6 membered heteroaryl, C₃₋₇ cycloalkyl-C₁₋₄ alkyl-, phenyl-C₁₋₄ alkyl-, 4-7 membered heterocyclyl-C₁₋₄ alkyl-, and 5-6 membered heteroaryl-C₁₋₄ alkyl- are each substituted with 0, 1, 2, 3, or 4 substituents independently selected from C₁₋₄ alkyl, C₃₋₇ cycloalkyl, cyclopropyl, oxo, —C(O)C₁₋₄alkyl, —C(O)OC₁₋₄alkyl, —C(O)NH₂, —OH, —F, —Cl, —O—C₁₋₄alkyl and —CN;
each instance of R^{A} is independently selected from —D, halo, CN, C₁₋₄ alkyl, C₁₋₄ haloalkyl, C₃₋₆ cycloalkyl, 4-6 membered heterocyclyl, OR^{a1}, SR^{a1}, SF₅, NR^{a1}R^{a1}, C₃₋₆ cycloalkyl-C₁₋₃ alkyl-, and 4-6 membered heterocyclyl-C₁₋₃ alkyl-, wherein said C₁₋₄ alkyl, C₁₋₄ haloalkyl-, C₃₋₆ cycloalkyl, 4-6 membered heterocyclyl-, C₃₋₆ cycloalkyl-C₁₋₃ alkyl-, and 4-6 membered heterocyclyl-C₁₋₃ alkyl are substituted with 0, 1, 2, 3, or 4 substituents independently selected from —D, halo, —OH, —C₁₋₄ alkyl and —OC₁₋₄ alkyl;
or, alternatively, two R^{A} groups on adjacent atoms of Ring A, together with the ring atoms to which they are attached, form Ring D, wherein Ring D is selected from C₃₋₆ cycloalkyl, 4-6 membered heterocyclyl, phenyl, and 5-6 membered heteroaryl, each of which is substituted with 0, 1, 2, 3, or 4 substituents independently selected from —D, halo, —OH, —C₁₋₄ alkyl and —OC₁₋₄ alkyl;
each R^{a1} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocyclyl, 5-10 membered heteroaryl, C₃₋₁₀ cycloalkyl-C₁₋₄ alkyl-, 6-10 membered aryl-C₁₋₄ alkyl-, 4-10 membered heterocyclyl-C₁₋₄ alkyl-, and 5-10 membered heteroaryl-C₁₋₄ alkyl-, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocyclyl, 5-10 membered heteroaryl, C₃₋₁₀ cycloalkyl-C₁₋₄ alkyl, 6-10 membered aryl-C₁₋₄ alkyl, 4-10 membered heterocyclyl-C₁₋₄ alkyl, and 5-10 membered heteroaryl-C₁₋₄ alkyl are each substituted with 0, 1, 2, 3, or 4 independently selected R² substituents; or two R^{a1} groups attached to the same nitrogen atom together with the nitrogen to which they are attached form a 4-7-membered heterocyclyl group substituted with 0, 1, 2, 3, or 4 substituents independently selected from —D, halo, —OH, —C₁₋₄ alkyl and —OC₁₋₄ alkyl;
each R^{b1} is independently selected from C₁₋₆ alkyl, C₁₋₆ hydroxyalkyl, C₃₋₉ cycloalkyl and C₂₋₆ heteroalkyl;
each R^{a2} is independently selected from H, C₁₋₆ alkyl, C₁₋₆ hydroxyalkyl, C₃₋₉ cycloalkyl and C₂₋₆ heteroalkyl, or, when possible, two instances of R^{a2} and the atom to which they are attached are taken together to form a 4-7 member heterocycle substituted with 0, 1, 2, 3, or 4 substituents independently selected from —D, halo, —OH, —C₁₋₄ alkyl and —OC₁-4 alkyl;
each R^{b2} is independently selected from C₁₋₆ alkyl, C₁₋₆ hydroxyalkyl, C₃₋₉ cycloalkyl and C₂₋₆ heteroalkyl;
X is X¹ when Ring A is heterocyclyl and is selected from X¹ and X² when Ring A is aryl or heteroaryl;
X¹ is selected from —S(O)₂— and —C(O)—;
X² is selected from —O—, —NH—, —N(CH₃)— and —CH₂—;
L is a covalent bond or a bivalent, saturated or unsaturated, straight or branched C₁. ₅₀ hydrocarbon chain, wherein 0-6 methylene units of L are independently replaced by —Cy—, —CH(R)—, —C(R)₂—, —O—, —NR—, —S—, —OC(═O)—, —C(═O)O—, —C(═O)—, —S(═O)—, —S(═O)₂—, —NRS(═O)₂—, —S(═O)₂NR—, —NRC(═O)—, —C(═O)NR—, —OC(═O)NR— or —NRC(═O)O—, wherein:
each —Cy— is independently a bivalent ring selected from phenylene, an 8-10 membered bicyclic arylene, a 4-7 membered monocyclic carbocyclylene, a 5-11 membered spiro carbocyclylene, a 4-10 membered bicyclic carbocyclylene, a 5-10 membered bridged carbocyclylene, a 4-7 membered monocyclic heterocyclylene having 1-2 heteroatoms independently selected from nitrogen, oxygen, and sulfur, a 5-11 membered spiro heterocyclylene having 1-2 heteroatoms independently selected from nitrogen, oxygen, and sulfur, an 4-10 membered bicyclic heterocyclylene having 1-2 heteroatoms independently selected from nitrogen, oxygen, and sulfur, a 5-10 membered bridged bicyclic saturated or partially unsaturated heterocyclylene having 1-2 heteroatoms independently selected from nitrogen, oxygen, and sulfur, a 5-6 membered heteroarylene having 1-4 heteroatoms independently selected from nitrogen, oxygen, and sulfur, or an 8-10 membered bicyclic heteroarylene having 1-5 heteroatoms independently selected from nitrogen, oxygen, or sulfur, wherein each phenylene, arylene, carbocyclylene, heterocyclylene and heteroarylene is substituted with 0, 1, 2, 3, or 4 instances of R^{C};
LBM is selected from:

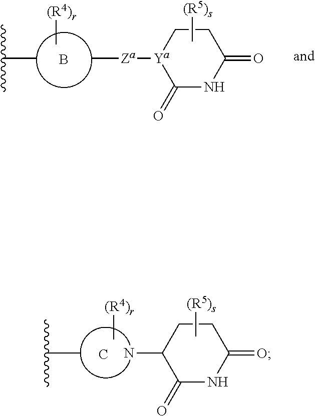

Y^{a} is CH or N;
Z^{a} is a bond, —CH₂—, —NH—, O, or —NHC(O)— where NH of —NHC(O)— is attached to Y^{a};
Ring B is phenylene, a 4-10-membered heterocyclylene, a 5-6-membered monocyclic heteroarylene or a 9-10-membered fused bicyclic heteroarylene, wherein each heteroarylene contains one to three nitrogen ring atoms.
ring C together with the (R⁴), substituents is selected from the group consisting of:

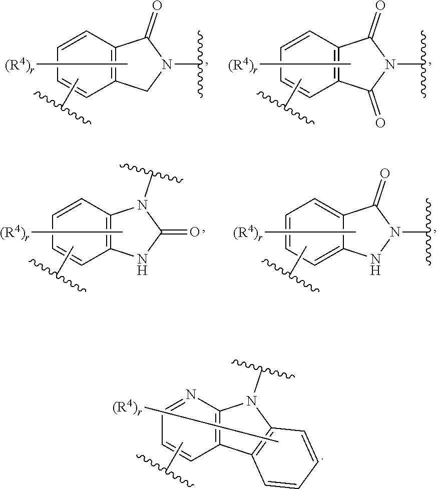

each instance of R^{C} is independently selected from —D, halogen, —OH, and —C₁₋₆ alkyl;
each instance of R⁴ is independently selected from —D, halogen, —OH, and —C₁₋₆ alkyl;
each instance of R⁵ is independently selected from —D, halogen, —OH, and —C₁₋₆ alkyl;
each instance of R is independently selected from hydrogen and —C₁₋₆ alkyl;
n is 0, 1, 2, 3, or 4;
r is 0, 1, 2, 3, or 4; and
s is 0, 1, 2, 3, or 4.

In an embodiment, provided is a compound of Formula A-I:

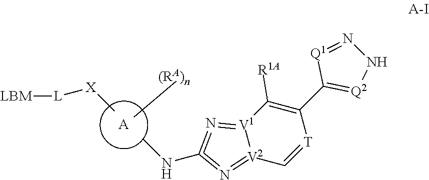

or a pharmaceutically acceptable salt thereof, wherein Ring A, R^{1A}, R^{A}, X, V¹, V², T, Q¹, Q², L, LBM and n are as defined in any of the embodiments described herein.

In one aspect, provided is a compound of Formula I

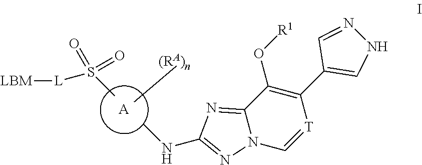

or a pharmaceutically acceptable salt thereof, wherein:
Ring A is selected from

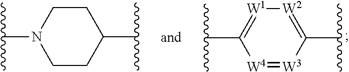

W¹, W², W³ and W⁴ are each independently CH or N;
R¹ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocyclyl, 5-10 membered heteroaryl, C₃₋₁₀ cycloalkyl-C₁₋₄ alkyl-, 6-10 membered aryl-C₁₋₄ alkyl-, 4-10 membered heterocyclyl-C₁₋₄ alkyl-, and 5-10 membered heteroaryl-C₁₋₄ alkyl-, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocyclyl, 5-10 membered heteroaryl, C₃₋₁₀ cycloalkyl-C₁₋₄ alkyl, 6-10 membered aryl-C₁₋₄ alkyl, 4-10 membered heterocyclyl-C₁₋₄ alkyl, and 5-10 membered heteroaryl-C₁₋₄ alkyl are each substituted with 0, 1, 2, 3, or 4 independently selected R² substituents;
each R² is independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocyclyl, 5-6 membered heteroaryl, C₃₋₇ cycloalkyl-C₁₋₄ alkyl-, phenyl-C₁₋₄ alkyl-, 4-7 membered heterocyclyl-C₁₋₄ alkyl-, 5-6 membered heteroaryl-C₁₋₄ alkyl-, OR^{a2}, SR^{a2}, NHOR^{a2}, C(O)R^{b2}, C(O)NR^{a2}R^{a2}, C(O)NR^{a2}(OR^{a2}), C(O)OR^{a2}, OC(O)R^{b2}, OC(O)NR^{a2}R^{a2}, NR^{a2}R^{a2}, NR^{a2}NR^{a2}R^{a2}, NR^{a2}C(O)R^{b2}, NR^{a2}C(O)OR^{a2}, NR^{a2}C(O)NR^{a2}R^{a2}, C(═NR^{a2})R^{b2}, C(═NR^{a2})NR^{a2}R^{a2}, NR^{a2}C(═NR^{a2})NR^{a2}R^{a2}, NR^{a2}C(═NR^{a2})R^{b2}, NR^{a2}S(O)NR^{a2}R^{a2} NR^{a2}S(O)R^{b2}, NR^{a2}S(O)₂R^{b2}, NR^{a2}S(O)(═NR^{a2})R^{b2}, NR^{a2}S(O)₂NR^{a2}R^{a2}, S(O)R^{b2}, S(O)NR^{a2}R^{a2}, S(O)₂R^{b2}, S(O)₂NR^{a2}R^{a2}, OS(O)(═NR^{a2})R^{b2}, OS(O)₂R^{b2}, S(O)(═NR^{a2})R^{b2}, SF₅, P(O)R^{a2}R^{a2}, OP(O)(OR^{a2})(OR^{a2}), P(O)(OR^{a2})(OR^{a2}), and BR^{a2}R^{a2}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocyclyl, 5-6 membered heteroaryl, C₃₋₇ cycloalkyl-C₁₋₄ alkyl, phenyl-C₁₋₄ alkyl, 4-7 membered heterocyclyl-C₁₋₄ alkyl, and 5-6 membered heteroaryl-C₁₋₄ alkyl are each optionally substituted with 1, 2, 3, or 4 substituents independently selected from C₁₋₄ alkyl, C₃₋₇ cycloalkyl, cyclopropyl, oxo, —C(O)C₁₋₄alkyl, —C(O)OC₁₋₄alkyl, —C(O)NH₂, —OH, —F, —Cl, —O—C₁₋₄alkyl and —CN;
each R^{a2} is independently selected from H, C₁₋₆ alkyl, C₁₋₆ hydroxyalkyl, C₃₋₉ cycloalkyl and C₂₋₆ heteroalkyl, or, when possible, two instances of R^{a2} and the atom to which they are attached are taken together to form a saturated 3-7-membered heterocycle;
each R^{b2} is independently selected from C₁₋₆ alkyl, C₁₋₆ hydroxyalkyl, C₃₋₉ cycloalkyl and C₂₋₆ heteroalkyl;
L is a covalent bond or a bivalent, saturated or unsaturated, straight or branched C₁₋₅₀ hydrocarbon chain, wherein 0-6 methylene units of L are independently replaced by —Cy—, —CH(R)—, —C(R)₂—, —O—, —NR—, —S—, —OC(═O)—, —C(═O)O—, —C(═O)—, —S(═O)—, —S(═O)₂—, —NRS(═O)₂—, —S(═O)₂NR—, —NRC(═O)—, —C(═O)NR—, —OC(═O)NR— or —NRC(═O)O—, wherein:
each —Cy— is independently a bivalent ring selected from phenylene, an 8-10 membered bicyclic arylene, a 4-7 membered monocyclic carbocyclylene, a 5-11 membered spiro carbocyclylene, a 4-10 membered bicyclic carbocyclylene, a 5-10 membered bridged carbocyclylene, a 4-7 membered monocyclic heterocyclylene having 1-2 heteroatoms independently selected from nitrogen, oxygen, and sulfur, a 5-11 membered spiro heterocyclylene having 1-2 heteroatoms independently selected from nitrogen, oxygen, and sulfur, an 4-10 membered bicyclic heterocyclylene having 1-2 heteroatoms independently selected from nitrogen, oxygen, and sulfur, a 5-10 membered bridged bicyclic saturated or partially unsaturated heterocyclylene having 1-2 heteroatoms independently selected from nitrogen, oxygen, and sulfur, a 5-6 membered heteroarylene having 1-4 heteroatoms independently selected from nitrogen, oxygen, and sulfur, or an 8-10 membered bicyclic heteroarylene having 1-5 heteroatoms independently selected from nitrogen, oxygen, or sulfur, wherein each phenylene, arylene, carbocyclylene, heterocyclylene and heteroarylene is substituted with 0, 1, 2, 3, or 4 instances of R^{C};
LBM is selected from:

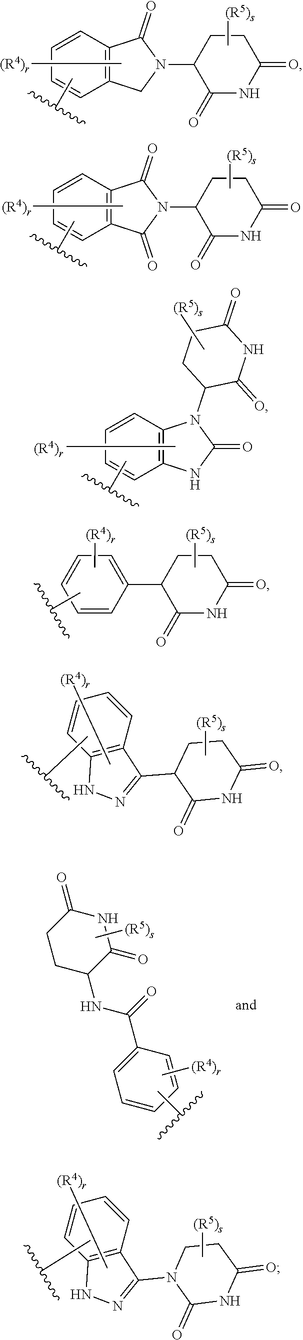

each instance of R^{A} is independently selected from —D, halogen, —C₁₋₆ alkyl, —OH and —OC₁₋₄ alkyl, wherein each —C₁₋₆ alkyl is substituted with 0, 1, 2 or 3 groups independently selected from —D, —F, —OH and —OC₁₋₄ alkyl; or two R^{A} groups on adjacent atoms of Ring A, together with the ring atoms to which they are attached, form Ring D, wherein Ring D is selected from C₃₋₆ cycloalkyl, 4-6 membered heterocyclyl, phenyl, and 5-6 membered heteroaryl, each of which is substituted with 0, 1, 2, 3, or 4 substituents independently selected from —D, halo, —OH, —C₁₋₄ alkyl and —OC₁₋₄ alkyl;
each instance of R^{C} is independently selected from —D, halogen, —OH, and —C₁₋₆ alkyl;
each instance of R⁴ is independently selected from —D, halogen, —OH, and —C₁₋₆ alkyl;
each instance of R⁵ is independently selected from —D, halogen, —OH, and —C₁₋₆ alkyl;
each instance of R is independently selected from hydrogen and —C₁₋₆ alkyl;
n is 0, 1, 2, 3, or 4;
r is 0, 1, 2, 3, or 4; and
s is 0, 1, 2, 3, or 4.

In an embodiment, provided is a compound of Formula I:

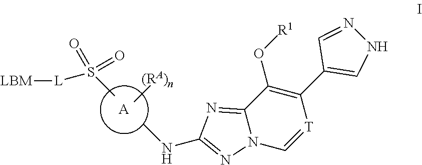

or a pharmaceutically acceptable salt thereof, wherein Ring A, T, R¹, R^{A}, L, LBM and n are as defined in any of the embodiments described herein.

As generally defined herein, Ring A is selected from

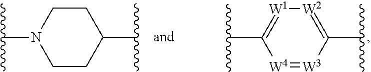

wherein W¹, W², W³ and W⁴ are as defined in any of the embodiments described herein. In an embodiment, ring A is selected from

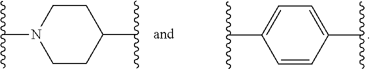 [SMILES: *C1CCN(*)CC1 ; *c1ccc(*)cc1]

In an embodiment, Ring A is

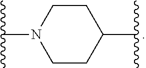 [SMILES: *C1CCN(*)CC1]

In an embodiment, Ring A is

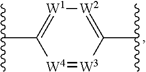

wherein W¹, W², W³ and W⁴ are as defined in any of the embodiments described herein. In an embodiment, ring A is

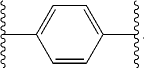 [SMILES: *c1ccc(*)cc1]

As generally defined herein, V¹ is nitrogen and V² is carbon, and Ring

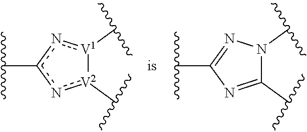

or V² is nitrogen and V¹ is carbon, and Ring

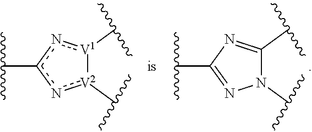

In one embodiment, V¹ is nitrogen and V² is carbon, and Ring

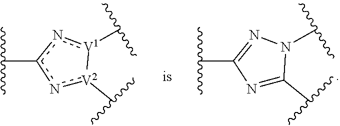

In one embodiment, V² is nitrogen and V¹ is carbon, and Ring

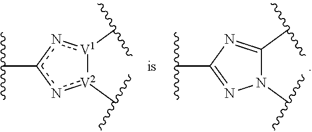

As generally defined herein, T is CH or N. In one embodiment, T is CH. In one embodiment, T is N.

As generally defined herein, Q¹ and Q² are independently selected from N and CH. In an embodiment, Q¹ and Q² are both CH. In an embodiment, Q¹ and Q² are both N. In an embodiment, Q¹ is N and Q² is CH. In an embodiment, Q² is N and Q¹ is CH.

As generally defined herein, X is X¹ when Ring A is heterocyclyl and is selected from X¹ and X² when Ring A is aryl or heteroaryl, wherein X¹ and X² are as defined in any of the embodiments described herein. In an embodiment, X is X¹. In an embodiment, Ring A is aryl or heteroaryl and X is X². In an embodiment, X is selected from —S(O)₂— and —C(O)—. In an embodiment, X is —S(O)₂—. In an embodiment, X is C(O)—. In an embodiment, X is —O—. In an embodiment, X is —NH—. In an embodiment, X is —N(CH₃)—. In an embodiment, X is —CH₂—.

As generally defined herein, X¹ is selected from —S(O)₂— and —C(O)—. In an embodiment, X¹ is —S(O)₂—. In an embodiment, X¹ is C(O)—.

As generally defined herein, X² is selected from —O—, —NH—, —N(CH₃)— and —CH₂—. In an embodiment, X² is —O—. In an embodiment, X² is —NH—. In an embodiment, X² is —N(CH₃)—. In an embodiment, X² is —CH₂—.

As generally defined herein, Y^{a} is CH or N. In an embodiment, Y^{a} is CH. In an embodiment, Y^{a} is N.

As generally defined herein, Z^{a} is a bond, —CH₂—, —NH—, 0, or —NHC(O)— where NH of —NHC(O)— is attached to Y^{a}. In an embodiment, Z^{a} is selected from the group consisting of a bond, —NH—, —O— and —NHC(O)—. In an embodiment, Z^{a} is selected from the group consisting of a bond, —NH— and —NHC(O)—. In an embodiment, Z^{a} is selected from the group consisting of —NH—, and —NHC(O)—. In an embodiment, Z^{a} is a bond. In an embodiment, Z^{a} is —CH₂—. In an embodiment, Z^{a} is —NH—. In an embodiment, Z^{a} is 0. In an embodiment, Z^{a} is —NHC(O)— where NH of —NHC(O)— is attached to Y^{a}.

As generally defined herein, Ring B is phenylene, a 4-10-membered heterocyclylene, a 5-6-membered monocyclic heteroarylene or a 9-10-membered fused bicyclic heteroarylene, wherein each heteroarylene contains one to three nitrogen ring atoms. In an embodiment, Ring B is phenylene. In an embodiment, Ring B is a 4-10 membered heterocyclylene containing 1, 2 or 3 heteroatoms selected from O, N and S and oxidized forms thereof. In an embodiment, Ring B is a 5-6-membered monocyclic heteroarylene containing 1, 2, 3 or 4 heteroatoms selected from O, N and S and oxidized forms thereof. In an embodiment, Ring B is a 9-10-membered fused bicyclic heteroarylene containing 1, 2, 3 or 4 heteroatoms selected from O, N and S and oxidized forms thereof.

As generally defined herein, ring C together with the (R⁴)r substituents is selected from the group consisting of:

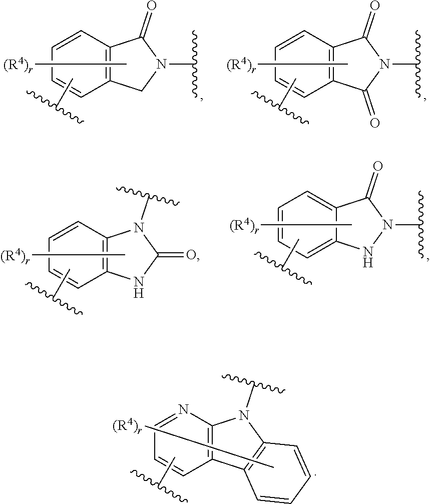

As generally defined herein, R^{1A} is independently selected from H, D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocyclyl, 5-10 membered heteroaryl, C₃₋₁₀ cycloalkyl-C₁₋₄ alkyl, 6-10 membered aryl-C₁₋₄ alkyl, 4-10 membered heterocyclyl-C₁₋₄ alkyl, 5-10 membered heteroaryl-C₁₋₄ alkyl, OR^{a1}, SR^{a1}, NHOR^{a1}, C(O)R^{a1}, C(O)NR^{a1}R^{a1}, C(O)NR^{a1}(OR^{a1}), C(O)OR^{a1}, OC(O)R^{a1}, OC(O)NR^{a1}R^{a1}, NR^{a1}R^{a1}, NR^{a1}NR^{a1}R^{a1}, NR^{a1}C(O)R^{b1}, NR^{a1}C(O)OR^{a1}, NR^{a1}C(O)NR^{a1}R^{a1}, C(═NR^{a1})R^{b1}, C(═NR^{a1})NR^{a1}R^{a1}, NR^{a1}C(═NR^{a1})NR^{a1}R^{a1}, NR^{a1}C(═NR^{a1})R^{b1}, NR^{a1}S(O)NR^{a1}R^{a1}, NR^{a1}S(O)R^{b1}, NR^{a1}S(O)₂R^{b1}, NR^{a1}S(O)(═NR^{a1})R^{b1}, NR^{a1}S(O)₂NR^{a1}R^{a1}, S(O)R^{b1}, S(O)NR^{a1}R^{a1}, S(O)₂R^{b1}, S(O)₂NR^{a1}R^{a1}, OS(O)(═NR^{a1})R^{b1}, OS(O)₂R^{b1}, S(O)(═NR^{a1})R^{b1}, SFS, P(O)R^{a1}R^{b1}, OP(O)(OR^{a1})(OR^{a1}) and P(O)(OR^{a1})(OR^{a1}), wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocyclyl, 5-10 membered heteroaryl, C₃₋₁₀ cycloalkyl-C₁₋₄ alkyl, 6-10 membered aryl-C₁₋₄ alkyl, 4-10 membered heterocyclyl-C₁₋₄ alkyl, and 5-10 membered heteroaryl-C₁₋₄ alkyl are each substituted with 0, 1, 2, 3, or 4 independently selected R² substituents, wherein each R^{a1}, R^{b1} and R² are as defined in any of the embodiments described herein.

In an embodiment, R^{1A} is independently selected from H, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocyclyl, 5-6 membered heteroaryl, C₃₋₇ cycloalkyl-C₁₋₄ alkyl-, phenyl-C₁₋₄ alkyl-, 4-7 membered heterocyclyl-C₁₋₄ alkyl-, 5-6 membered heteroaryl-C₁₋₄ alkyl-, OR^{a1}, SR^{a1} C(O)R^{b1}, C(O)NR^{a1}R^{a1}, C(O)OR^{a1}, OC(O)R^{b1}, OC(O)NR^{a1}R^{a1}, NR^{a1}R^{a1}, NR^{a1}C(O)R^{b1}, NR^{a1}C(O)OR^{a1}, NR^{a1}C(O)NR^{a1}R^{a1}, NR^{a1}S(O)₂R^{b1}, NR^{a1}S(O)₂NR^{a1}R^{a1}, S(O)₂R^{b1}, and S(O)₂NR^{a1}R^{a1}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocyclyl, 5-6 membered heteroaryl, C₃₋₇ cycloalkyl-C₁₋₄ alkyl-, phenyl-C₁₋₄ alkyl-, 4-7 membered heterocyclyl-C₁₋₄ alkyl-, and 5-6 membered heteroaryl-C₁₋₄ alkyl- are each substituted with 0, 1, 2, 3, or 4 independently selected R² substituents, wherein each R^{a1}, R^{b1} and R² are as defined in any of the embodiments described herein.

In an embodiment, R^{1A} is independently selected from H, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocyclyl, 5-6 membered heteroaryl, C₃₋₇ cycloalkyl-C₁₋₄ alkyl-, phenyl-C₁₋₄ alkyl-, 4-7 membered heterocyclyl-C₁₋₄ alkyl-, 5-6 membered heteroaryl-C₁₋₄ alkyl-, OR^{a1}, C(O)R^{b1}, C(O)NR^{a1}R^{a1}, C(O)OR^{a1}, OC(O)R^{b1}, OC(O)NR^{a1}R^{a1}, NR^{a1}R^{a1}, NR^{a1}C(O)R^{b1}, NR^{a1}C(O)OR^{a1}, NR^{a1}C(O)NR^{a1}R^{a1}, NR^{a1}S(O)₂R^{b1}, NR^{a1}S(O)₂NR^{a1}R^{a1}, S(O)₂R^{b1}, and S(O)₂NR^{a1}R^{a1}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocyclyl, 5-6 membered heteroaryl, C₃₋₇ cycloalkyl-C₁₋₄ alkyl-, phenyl-C₁₋₄ alkyl-, 4-7 membered heterocyclyl-C₁₋₄ alkyl-, and 5-6 membered heteroaryl-C₁₋₄ alkyl- are each substituted with 0, 1, 2, 3, or 4 independently selected R² 25 substituents, wherein each R^{a1}, R^{b1} and R² are as defined in any of the embodiments described herein.

In an embodiment, R^{1A} is independently selected from H, halo, CN, C₁₋₆ alkyl, C₁₋₆ haloalkyl, C₃₋₇ cycloalkyl, phenyl, 4-7 membered heterocyclyl, 5-6 membered heteroaryl, C₃₋₇ cycloalkyl-C₁₋₃ alkyl, phenyl-C₁₋₃ alkyl, 4-7 membered heterocyclyl-C₁₋₃ alkyl, 5-6 membered 30 heteroaryl-C₁₋₃ alkyl, OR^{a1}, SR^{a1}, and NR^{a1}R^{a1}, wherein said C₁₋₆ alkyl, C₁₋₆ haloalkyl, C₃₋₇ cycloalkyl, phenyl, 4-7 membered heterocyclyl, 5-6 membered heteroaryl, C₃₋₇ cycloalkyl-C₁₋₃ alkyl-, phenyl-C₁₋₃ alkyl-, 4-7 membered heterocyclyl-C₁₋₃ alkyl-, and 5-6 membered heteroaryl-C₁₋₃ alkyl are each substituted with 0, 1 or 2 independently selected R² substituents, wherein each R^{a1} and R² are as defined in any of the embodiments described herein.

In an embodiment, R^{1A} is independently selected from H, halo, CN, C₁₋₆ alkyl, C₁₋₆ haloalkyl, C₃₋₇ cycloalkyl, phenyl, 4-7 membered heterocyclyl, 5-6 membered heteroaryl, C₃₋₇ cycloalkyl-C₁₋₃ alkyl-, phenyl-C₁₋₃ alkyl-, 4-7 membered heterocyclyl-C₁₋₃ alkyl-, 5-6 membered heteroaryl-C₁₋₃ alkyl-, OR^{a1} and NR^{a1}R^{a1}, wherein said C₁₋₆ alkyl, C₁₋₆ haloalkyl, C₃₋₇ cycloalkyl, phenyl, 4-7 membered heterocyclyl, 5-6 membered heteroaryl, C₃₋₇ cycloalkyl-C₁₋₃ alkyl-, phenyl-C₁₋₃ alkyl-, 4-7 membered heterocyclyl-C₁₋₃ alkyl-, and 5-6 membered heteroaryl-C₁₋₃ alkyl are each substituted with 0, 1 or 2 independently selected R² substituents, wherein each R^{a1} and R² are as defined in any of the embodiments described herein.

In an embodiment, R^{1A} is independently selected from H, halo, CN, C₁₋₆ alkyl, C₂₋₆ alkenyl, C₂₋₆ alkynyl, C₁₋₆ haloalkyl, C₃₋₄ cycloalkyl, and OR^{a1}, wherein said C₁₋₆ alkyl, C₂₋₆ alkenyl, C₂₋₆ alkynyl, C₁₋₆ haloalkyl, and C₃-4 cycloalkyl are each substituted with 0, 1, 2, 3, or 4 independently selected R² substituents, wherein each R^{a1} and R² are as defined in any of the embodiments described herein.

In an embodiment, R^{1A} is independently selected from H, halo, CN, C₁₋₆ alkyl, C₂₋₆ alkenyl, C₂₋₆ alkynyl, C₁₋₆ haloalkyl, C₃₋₄ cycloalkyl, and OR^{a1}, wherein said C₁₋₆ alkyl, C₂₋₆ alkenyl, C₂₋₆ alkynyl, C₁₋₆ haloalkyl, and C₃₋₄ cycloalkyl are each substituted with 0, 1 or 2 independently selected R² substituents, wherein each R^{a1} and R² are as defined in any of the embodiments described herein.

In an embodiment, R^{1A} is independently selected from H, C₁₋₆ alkyl, phenyl, 5-7 membered heterocyclyl, OR^{a1}, SR^{a1}, and NR^{a1}R^{a1}, wherein said C₁₋₆ alkyl, phenyl, and 5-7 membered heterocyclyl are each substituted with 0, 1 or 2 independently selected R² substituents, wherein each R^{a1} and R² are as defined in any of the embodiments described herein.

In an embodiment, R^{1A} is independently selected from H, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocyclyl, 5-6 membered heteroaryl, C₃₋₇ cycloalkyl-C₁₋₄ alkyl-, phenyl-C₁₋₄ alkyl-, 4-7 membered heterocyclyl-C₁₋₄ alkyl-, 5-6 membered heteroaryl-C₁₋₄ alkyl-, OR^{a1}, SR^{a1}, and NR^{a1}R^{a1} wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocyclyl, 5-6 membered heteroaryl, C₃₋₇ cycloalkyl-C₁₋₄ alkyl-, phenyl-C₁₋₄ alkyl-, 4-7 membered heterocyclyl-C₁₋₄ alkyl-, and 5-6 membered heteroaryl-C₁₋₄ alkyl- are each substituted with 0, 1, 2, 3, or 4 independently selected R² substituents, wherein each Rai and R² are as defined in any of the embodiments described herein.

In an embodiment, R^{1A} is H, C₁₋₆ alkyl, phenyl, 5-7 membered heterocyclyl, OR^{a1}SR^{a1}, or NR^{a1}R^{a1}, wherein said C₁₋₆ alkyl, phenyl, and 5-7 membered heterocyclyl are each substituted with 0, 1 or 2 independently selected R² substituents, wherein each R^{a1}, and R² are as defined in any of the embodiments described herein.

In an embodiment, R^{1A} is phenyl substituted with 0, 1 or 2 independently selected R² substituents, wherein each R² is as defined in any of the embodiments described herein.

In an embodiment, R^{1A} is selected from 6-10 membered aryl, 4-10 membered heterocyclyl, 5-10 membered heteroaryl, OR^{a1} and NR^{a1}R^{a1}, wherein said 6-10 membered aryl, 4-10 membered heterocyclyl, and 5-10 membered heteroaryl are each substituted with 0, 1, 2, 3 or 4 independently selected R² substituents, wherein each R^{a1} and R² are as defined in any of the embodiments described herein.

In an embodiment, R^{1A} is independently selected from phenyl, 4-10 membered heterocyclyl, 5-10 membered heteroaryl, OR^{a1}, and NR^{a1}R^{a1}, wherein said phenyl, 4-10 membered heterocyclyl, and 5-10 membered heteroaryl are each substituted with 0, 1, 2, 3, or 4 independently selected R² substituents, wherein each R^{a1}, and R² are as defined in any of the embodiments described herein.

In an embodiment, R^{1A} is independently selected from 4-10 membered heterocyclyl and OR^{a1}, wherein said 4-10 membered heterocyclyl is substituted with 0, 1, 2, 3, or 4 independently selected R² substituents, wherein each R^{a1} and R² are as defined in any of the embodiments described herein.

In an embodiment, R^{1A} is 5-7 membered heterocyclyl substituted with 0, 1 or 2 independently selected R² substituents, wherein each R² is as defined in any of the embodiments described herein.

In an embodiment, R^{1A} is independently selected from phenyl, 4-10 membered heterocyclyl, 5-10 membered heteroaryl, and OR^{a1}, wherein said phenyl, 4-10 membered heterocyclyl, and 5-10 membered heteroaryl are each substituted with 0, 1, 2, 3, or 4 independently selected R² substituents, wherein each Rai and R² are as defined in any of the embodiments described herein.

In an embodiment, R^{1A} is selected from OR^{a1}, phenyl, pyridin-4-yl, 2-oxy-benzo[d]oxazol-(3H)-7-yl, and 1H-indazol-5-yl, wherein said phenyl, pyridin-4-yl, 2-oxy-benzo[d]oxazol-(3H)-7-yl, and 1H-indazol-5-yl are each substituted with 0, 1, 2, 3, or 4 independently selected R² substituents, wherein each R^{a1} and R² are as defined in any of the embodiments described herein.

In an embodiment, R^{1A} is selected from phenyl, 5-10 membered heteroaryl, and OR^{a1}, wherein said phenyl and 5-10 membered heteroaryl are each substituted with 0, 1, 2, 3, or 4 independently selected R² substituents, wherein each R^{a1} and R² are as defined in any of the embodiments described herein.

In an embodiment, R^{1A} is selected from phenyl, 4-fluorophenyl, 3-trifluoromethylphenyl, and 2-fluoro-3-cyano-phenyl.

In an embodiment, R^{1A} is independently selected from H and OR^{a} wherein each R^{a1} is as defined in any of the embodiments described herein.

In an embodiment, R^{1A} is C₁₋₃ alkyl.

In an embodiment, R^{1A} is selected from propyl and isopropyl.

In an embodiment, R^{1A} is selected from pyrrolidin-1-yl, 3,3-difluoropyrrolidin-1-yl, piperidin-1-yl, 3-fluoropiperidin-1-yl, 4-fluoropiperidin-1-yl, 4-methylpiperidin-lyl, (4-trifluoromethyl)piperidin-1-yl, 3,3-difluoropiperidin-1-yl, 3-(difluoromethyl)pyrrolidinyl, 2-methylpyrrolidinyl, 2-methylpiperidinyl, 3-(trifluoromethyl)piperidinyl, azabicyclo[2.2.1]heptan-7-yl, azabicyclo[2.2.1]heptan-2-yl, and (2-methoxyethyl)piperazin-1-yl.

In an embodiment, R^{1A} is SR^{a1} wherein each R^{a1} is as defined in any of the embodiments described herein.

In an embodiment, R^{1A} is NR^{a1}R^{a1} wherein each R^{a1} is as defined in any of the embodiments described herein.

In an embodiment, R^{1A} is OR^{a1} wherein each R^{a1} is as defined in any of the embodiments described herein.

In an embodiment, R^{1A} is selected from ethoxy, propoxy, isopropoxy, butoxy, isobutoxy, pentoxy, —OCH₂CF₃, —OCH₂CHF₂, —OCH₂CH₂CF₃, —OCH₂CF₂CHF₂, —OCH₂CH₂OCH₃, —OCD₂CD₃, —OCD(CH₃)₂, —OCH(CD₃)₂, —OCD(CD₃)₂, cyclopropoxy, cyclobutoxy, 3-methylcyclobutoxy, 3-difluoromethylcyclobutoxy, 3,3-difluorocyclobutoxy, cyclopentoxy, 3,3-difluorocyclopentoxy, 4,4-difluorocyclohexyloxy, tetrahydro-1H-pyran-4-oxy, tetrahydro-2H-pyran-4-oxy, 2-methyltetrahydro-2H-pyran-4-oxy, 3-methyltetrahydro-2H-pyran-4-oxy, 2-(trifluoromethyl)tetrahydro-2H-pyran-4-oxy, tetrahydrofuran-3-oxy, MeO—C(O)-piperidin-4-oxy, cyclopropyl-CH₂—O—, cyclobutyl-CH₂—O—, 1-trifluoromethylcyclobutyl-CH₂—O—, cyclopentyl-CH₂—O—and (tetrahydrofuran-3-yl)—CH₂—O

In an embodiment, R^{1A} is selected from ethoxy, isopropoxy, isobutoxy, tetrahydro-1H-pyran-4-oxy, cyclopropyl-CH₂—O—, (tetrahydrofuran-3-yl)—CH₂—O—, —OCH₂CH₂OCH₃, —OCH₂CF₃ and —OCH₂CHF₂.

In an embodiment, R^{1A} is selected from ethoxy, isopropoxy, isobutoxy, cyclobutoxy, —OCH₂CF₃ and —OCH₂CHF₂.

In an embodiment, R^{1A} is selected from isopropoxy, cyclobutoxy, —OCH₂CF₃ and —OCH₂CHF₂.

In an embodiment, R^{1A} is C₃₋₆ cycloalkoxy. In an embodiment, R^{1A} is cyclobutoxy. In an embodiment, R^{1A} is C₁₋₃ fluoroalkoxy. In an embodiment, R^{1A} is —OCH₂CF₃. In an embodiment, R^{1A} is C₁₋₃ alkoxy. In an embodiment, R^{1A} is —O′Pr.

As generally defined herein, R¹ is independently selected from H, C₁₋₆ alkyl, C₁₋₆ haloalkyl, C₃₋₁₀ cycloalkyl, 4-10 membered heterocyclyl, C₃₋₁₀ cycloalkyl-C₁₋₄ alkyl- and 4-10 membered heterocyclyl-C₁₋₄ alkyl-, wherein said C₁₋₆ alkyl, C₁₋₆ haloalkyl, C₃₋₁₀ cycloalkyl, 4-10 membered heterocyclyl, C₃₋₁₀ cycloalkyl-C₁₋₄ alkyl- and 4-10 membered heterocyclyl-C₁₋₄ alkyl-, are each substituted with 0, 1, 2, 3, or 4 independently selected R² substituents, wherein each R² is as defined in any of the embodiments described herein.

In an embodiment, R¹ is selected from C₁₋₆ alkyl, C₁₋₆ haloalkyl and C₃₋₆ cycloalkyl, wherein said C₁₋₆ alkyl, C₁₋₆ haloalkyl and C₃₋₆ cycloalkyl are each substituted with 0, 1 or 2 independently selected R² substituents, wherein each R² is as defined in any of the embodiments described herein.

In an embodiment, R¹ is selected from C₁₋₄ alkyl, C₁₋₃ fluoroalkyl and C₃₋₆ cycloalkyl, wherein said C₁₋₄ alkyl, C₁₋₃ fluoroalkyl and C₃₋₆ cycloalkyl are each substituted with 0, 1 or 2 independently selected R² substituents, wherein each R² is as defined in any of the embodiments described herein.

In an embodiment, R¹ is selected from —Me, —Et, —Pr, —′Pr, iso-butyl, sec-butyl, ′butyl, cyclopropyl, cyclobutyl, cyclopentyl, —CF₃, —CHF₂, CH₂CF₃, cyclopropyl, cyclobutyl, cyclopentyl and cyclohexyl, each substituted with 0, 1 or 2 independently selected R² substituents, wherein each R² is as defined in any of the embodiments described herein.

In an embodiment, R¹ is selected from C₁₋₆ alkyl, C₁₋₆ haloalkyl and C₃₋₆ cycloalkyl, wherein said C₁₋₆ alkyl, C₁₋₆ haloalkyl and C₃₋₆ cycloalkyl are each substituted with 0, 1 or 2 independently selected R² substituents, wherein each R² is as defined in any of the embodiments described herein.

In an embodiment, R¹ is selected from C₁₋₄ alkyl, C₁₋₃ fluoroalkyl and C₃₋₆ cycloalkyl, wherein said C₁₋₄ alkyl, C₁₋₃ fluoroalkyl and C₃₋₆ cycloalkyl are each substituted with 0, 1 or 2 independently selected R² substituents, wherein each R² is as defined in any of the embodiments described herein.

In an embodiment, R¹ is selected from —Me, —Et, —Pr, —′Pr, iso-butyl, sec-butyl, butyl, cyclopropyl, cyclobutyl, cyclopentyl, —CF₃, —CHF₂, CH₂CF₃, cyclopropyl, cyclobutyl, cyclopentyl and cyclohexyl, each substituted with 0, 1 or 2 independently selected R² substituents, wherein each R² is as defined in any of the embodiments described herein.

In an embodiment, R¹ is selected from ethyl, propyl, isopropyl, butyl, isobutyl, pentyl, —CH₂CF₃, —CH₂CHF₂, —CH₂CH₂CF₃, —CH₂CF₂CHF₂, —CH₂CH₂OCH₃, —CD₂CD₃, —CD(CH₃)₂, (CD₃)₂—CH—, (CD₃)₂—CD-, cyclopropyl, cyclobutyl, 3-methylcyclobutyl, 3-difluoromethylcyclobutyl, 3,3-difluorocyclobutyl, cyclopentyl, 3,3- difluorocyclopentyl, 4,4-difluorocyclohexyl, tetrahydro-1H-pyran-4-yl, tetrahydro-2H-pyran-4-yl, 2-methyltetrahydro-2H-pyran-4-yl, 3-methyltetrahydro-2H-pyran-4- yl, 2-(trifluoromethyl)tetrahydro-2H-pyran-4-yl, tetrahydrofuran-3-yl, MeO—C(O)-piperidin-4-yl, cyclopropyl-CH₂—, cyclobutyl-CH₂—, 1-trifluoromethylcyclobutyl-CH₂—, cyclopentyl-CH₂—, and (tetrahydrofuran-3-yl)—CH₂—.

In an embodiment, R¹ is selected from ethyl, isopropyl, isobutyl, tetrahydro-1H-pyran-4-yl, cyclopropyl-CH₂—, (tetrahydrofuran-3-yl)—CH₂—, —CH₂CH₂OCH₃, —CH₂CF₃, and —CH₂CHF₂.

In an embodiment, R¹ is selected from ethyl, isopropyl, isobutyl, cyclobutyl, —CH₂CF₃, and —CH₂CHF₂.

In an embodiment, R¹ is selected from isopropyl, cyclobutyl, —CH₂CF₃, and —CH₂CHF₂.

In an embodiment, R¹ is C₄₋₇ heterocyclyl. In an embodiment, R¹ is C₃₋₆ cycloalkyl. In an embodiment, R¹ is cyclobutyl. In an embodiment, R¹ is C₁₋₆ fluoroalkyl.

In an embodiment, R¹ is —CH₂CF₃. In an embodiment, R¹ is C₁₋₃ alkyl. In an embodiment, R¹ is —′Pr.

As generally defined herein, each R² is independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocyclyl, 5-6 membered heteroaryl, C₃₋₇ cycloalkyl-C₁₋₄ alkyl-, phenyl-C₁₋₄ alkyl-, 4-7 membered heterocyclyl-C₁₋₄ alkyl-, 5-6 membered heteroaryl-C₁₋₄ alkyl-, OR^{a2}, SR^{a2}, NHOR^{a2}, C(O)R^{b2}, C(O)NR^{a2}R^{a2}, C(O)NR^{a2}(OR^{a2}), C(O)OR^{a2}, OC(O)R^{b2}, OC(O)NR^{a2}R^{a2}, NR^{a2}R^{a2}, NR^{a2}NR^{a2}R^{a2}, NR^{a2}C(O)R^{b2}, NR^{a2}C(O)OR^{a2}, NR^{a2}C(O)NR^{a2}R^{a2} C(═NR^{a2})R^{b2}, C(═NR^{a2})NR^{a2}R^{a2}, NR^{a2}C(═NR^{a2})NR^{a2}R^{a2}, NR^{a2}C(═NR^{a2})R^{b2}, NR^{a2}S(O)NR^{a2}R^{a2}, NR^{a2}S(O)R^{b2}, NR^{a2}S(O)₂R^{b2}, NR^{a2}S(O)(═NR^{a2})R^{b2}, NR^{a2}S(O)₂NR^{a2}R^{a2} S(O)R^{b2}, S(O)NR^{a2}R^{a2}, S(O)₂R^{b2}, S(O)₂NR^{a2}R^{a2}, OS(O)(═NR^{a2})R^{b2}, OS(O)₂R^{b2}, S(O)(═NR^{a2})R^{b2}, SFS, P(O)R^{a2}R^{a2}, OP(O)(OR^{a2})(OR^{a2}) and P(O)(OR^{a2})(OR^{a2}), wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocyclyl, 5-6 membered heteroaryl, C₃₋₇ cycloalkyl-C₁₋₄ alkyl-, phenyl-C₁₋₄ alkyl-, 4-7 membered heterocyclyl-C₁₋₄ alkyl-, and 5-6 membered heteroaryl-C₁₋₄ alkyl- are each optionally substituted with 1, 2, 3, or 4 substituents independently selected from C₁₋₄ alkyl, C₃₋₇ cycloalkyl, cyclopropyl, oxo, —C(O)C₁₋₄alkyl, —C(O)OC₁₋₄alkyl, —C(O)NH₂, —OH, —F, —C₁, —O—C₁₋₄alkyl and —CN, wherein each R^{a2} and R^{b2} are as defined in any of the embodiments described herein.

In an embodiment, each R² is independently selected from H, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocyclyl, 5-6 membered heteroaryl, C₃₋₇ cycloalkyl-C₁₋₄ alkyl, phenyl-C₁₋₄ alkyl, 4-7 membered heterocyclyl-C₁₋₄ alkyl, 5-6 membered heteroaryl-C₁₋₄ alkyl, OR^{a2}, C(O)R^{b2}, C(O)NR^{a2}R^{a2}, C(O)OR^{a2}, OC(O)R^{b2}, OC(O)NR^{a2}R^{a2}, NR^{a2}R^{a2}, NR^{a2}C(O)R^{b2}, NR^{a2}C(O)OR^{a2}, NR^{a2}C(O)NR^{a2}R^{a2}, NR^{a2}S(O)₂R^{b2}, NR^{a2}S(O)₂NR^{a2}R^{a2}, S(O)₂R^{b2}, and S(O)₂NR^{a2}R^{a2}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocyclyl, 5-6 membered heteroaryl, C₃₋₇ cycloalkyl-C₁₋₄ alkyl, phenyl-C₁₋₄ alkyl, 4-7 membered heterocyclyl-C₁₋₄ alkyl, and 5-6 membered heteroaryl-C₁₋₄ alkyl are each substituted with 0, 1, 2, 3, or 4 substituents independently selected from C₁₋₄ alkyl, C₃₋₇ cycloalkyl, cyclopropyl, oxo, —C(O)C₁₋₄alkyl, —C(O)OC₁₋₄alkyl, —C(O)NH₂, —OH, —F, —Cl, —O—C₁₋₄alkyl and —CN, wherein each R^{a2} and R^{b2} are as defined in any of the embodiments described herein.

In an embodiment, each R² is independently selected from H, D, halo, CN, NO₂, C₁₋₆ alkyl, C₁₋₆ haloalkyl, C₃₋₇ cycloalkyl, phenyl, 4-7 membered heterocyclyl, 5-6 membered heteroaryl, C₃₋₇ cycloalkyl-C₁₋₄ alkyl, phenyl-C₁₋₄ alkyl, 4-7 membered heterocyclyl-C₁₋₄ alkyl, 5-6 membered heteroaryl-C₁₋₄ alkyl, OR^{a2}, C(O)R^{b2}, C(O)NR^{a2}R^{a2}, C(O)OR^{a2}, OC(O)R^{b2}, OC(O)NR^{a2}R^{a2}, NR^{a2}R^{a2}, NR^{a2}C(O)R^{b2}, NR^{a2}C(O)OR^{a2}, NR^{a2}C(O)NR^{a2}R^{a2}, NR^{a2}S(O)₂R^{b2}, NR^{a2}S(O)₂NR^{a2}R^{a2}, S(O)₂R^{b2}, and S(O)₂NR^{a2}R^{a2}, wherein said C₁₋₆ alkyl, C₁₋₆ haloalkyl, C₃₋₇ cycloalkyl, phenyl, 4-7 membered heterocyclyl, 5-6 membered heteroaryl, C₃₋₇ cycloalkyl-C₁₋₄ alkyl, phenyl-C₁₋₄ alkyl, 4-7 membered heterocyclyl-C₁₋₄ alkyl, and 5-6 membered heteroaryl-C₁₋₄ alkyl are each substituted with 0, 1, 2, 3, or 4 substituents independently selected from C₁₋₄ alkyl, C₃₋₇ cycloalkyl, cyclopropyl, oxo, —C(O)C₁₋₄alkyl, -5 C(O)OC₁₋₄alkyl, —C(O)NH₂, —OH, —F, —Cl, —O—C₁₋₄alkyl and —CN, wherein each R^{a2} and R^{b2} are as defined in any of the embodiments described herein.

In an embodiment, each R² is independently selected from H, halo, CN, NO₂, C₁₋₆ alkyl, C₁₋₆ haloalkyl, C₃₋₇ cycloalkyl, phenyl, 4-7 membered heterocyclyl, 5-6 membered heteroaryl, C₃₋₇ cycloalkyl-C₁₋₄ alkyl, phenyl-C₁₋₄ alkyl, 4-7 membered heterocyclyl-C₁₋₄ alkyl, 5-6 membered heteroaryl-C₁₋₄ alkyl, OR^{a2}, C(O)R^{b2}, C(O)NR^{a2}R^{a2}, C(O)OR^{a2}, OC(O)R^{b2}, OC(O)NR^{a2}R^{a2}, NR^{a2}R^{a2}, NR^{a2}C(O)R^{b2}, NR^{a2}C(O)OR^{a2}, NR^{a2}C(O)NR^{a2}R^{a2}, NR^{a2}S(O)₂R^{b2}, NR^{a2}S(O)₂NR^{a2}R^{a2}, S(O)₂R^{b2}, and S(O)₂NR^{a2}R^{a2}, wherein each R^{a2} and R^{b2} are as defined in any of the embodiments described herein.

In an embodiment, each R² is independently selected from halo, CN, C₁₋₆ alkyl, C₁₋₆ haloalkyl, C₃₋₆ cycloalkyl, 4-6 membered heterocyclyl, C₃₋₆ cycloalkyl-C₁₋₄ alkyl, 4-6 membered heterocyclyl-C₁₋₄ alkyl, OR^{a2}, NR^{a2}R^{a2} and S(O)₂R^{b2}, wherein said C₁₋₆ alkyl, C₁₋₆ haloalkyl, C₃₋₆ cycloalkyl, 4-6 membered heterocyclyl, C₃₋₆ cycloalkyl-C₁₋₄ alkyl, and 4-6 membered heterocyclyl-C₁₋₄ alkyl are each substituted with 0, 1, 2, 3, or 4 substituents independently selected from C₁₋₄ alkyl, C₃₋₇ cycloalkyl, cyclopropyl, oxo, —C(O)C₁₋₄alkyl, -20 C(O)OC₁₋₄alkyl, —C(O)NH₂, —OH, —F, —Cl, —O—C₁₋₄alkyl and —CN, wherein each R^{a2} and R^{b2} are as defined in any of the embodiments described herein.

In an embodiment, each R² is independently selected from halo, CN, C₁₋₆ alkyl, and 4-6 membered heterocyclyl-C₁₋₄ alkyl, wherein said C₁₋₆ alkyl and 4-6 membered heterocyclyl-C₁₋₄ alkyl are each substituted with 0, 1, 2, 3, or 4 substituents independently selected from C₁₋₄ alkyl, C₃₋₇ cycloalkyl, cyclopropyl, oxo, —C(O)C₁₋₄alkyl, —C(O)OC₁₋₄alkyl, —C(O)NH₂, —OH, —F, —Cl, —O—C₁₋₄alkyl and —CN.

In an embodiment, each R² is independently selected from H, halo, CN, C₁₋₄ alkyl, C₁₋₄ haloalkyl, OH, C₁₋₃ alkoxy, C₁₋₃ haloalkoxy, amino, C₁₋₃ alkylamino, di(C₁₋₃ alkyl)amino, cyano-C₁₋₄ alkyl, HO—C₁₋₄ alkyl, C₁₋₃ alkoxy-C₁₋₄ alkyl, and C₃₋₄ cycloalkyl.

In an embodiment, each R² is independently selected from H, D, halo, CN, C₁₋₆ alkyl, C₁₋₆ haloalkyl, OR^{a2}, and C(O)OR^{a2}, wherein said C₁₋₆ alkyl and C₁₋₆ haloalkyl are each substituted with 0, 1, 2, 3, or 4 substituents independently selected from C₁₋₄ alkyl, C₃₋₇ cycloalkyl, cyclopropyl, oxo, —C(O)C₁₋₄alkyl, —C(O)OC₁₋₄alkyl, —C(O)NH₂, —OH, —F, —Cl, —O—C₁₋₄alkyl and —CN, wherein each R^{a2} is as defined in any of the embodiments described herein.

In an embodiment, each R² is independently selected from H, D, halo, CN, C₁₋₆ alkyl, C₁₋₆ haloalkyl, C₃₋₄ cycloalkyl, OR^{a2}, C(O)R^{b2}, C(O)NR^{a2}R^{a2}, C(O)OR^{a2}, OC(O)R^{b2}, OC(O)NR^{a2}R^{a2}, NR^{a2}R^{a2}, NR^{a2}C(O)R^{b2}, NR^{a2}C(O)OR^{a2}, NR^{a2}C(O)NR^{a2}R^{a2}, NR^{a2}S(O)₂R^{b2}, NR^{a2}S(O)₂NR^{a2}R^{a2}, S(O)₂R^{b2}, and S(O)₂NR^{a2}R^{a2}, wherein said C₁₋₆ alkyl, C₁₋₆ haloalkyl, and C₃₋₄ cycloalkyl are each substituted with 0, 1, 2, 3, or 4 substituents independently selected from C₁₋₄ alkyl, C₃₋₇ cycloalkyl, cyclopropyl, oxo, —C(O)C₁₋₄alkyl, —C(O)OC₁₋₄alkyl, —C(O)NH₂, —OH, —F, —Cl, —O—C₁₋₄alkyl and —CN, wherein each R^{a2} and R^{b2} are as defined in any of the embodiments described herein.

In an embodiment, each R² is independently selected from D, halo, CN, C₁₋₆ alkyl, C₁₋₆ haloalkyl, C₃₋₇ cycloalkyl, OR^{a2}, C(O)R^{b2}, C(O)NR^{a2}R^{a2}, C(O)OR^{a2}, OC(O)R^{b2}, OC(O)NR^{a2}R^{a2}, NR^{a2}R^{a2}, NR^{a2}C(O)R^{b2}, NR^{a2}C(O)OR^{a2}, NR^{a2}C(O)NR^{a2}R^{a2}, NR^{a2}S(O)NR^{a2}R^{a2}, NR^{a2}S(O)R^{b2}, NR^{a2}S(O)₂R^{b2}, NR^{a2}S(O)₂NR^{a2}R^{a2}, S(O)R^{b2}, S(O)NR^{a2}R^{a2}, S(O)₂R^{b2}, S(O)₂NR^{a2}R^{a2}, SFS, wherein said C₁₋₆ alkyl, C₁₋₆ haloalkyl, C₃₋₇ cycloalkyl and 4-7 membered heterocyclyl are each optionally substituted with 1, 2, 3, or 4 substituents independently selected from C₁₋₄ alkyl, C₃₋₇ cycloalkyl, cyclopropyl, oxo, —C(O)C₁₋₄alkyl, —C(O)OC₁₋₄alkyl, —C(O)NH₂, —OH, —F, —Cl, —O—C₁₋₄alkyl and —CN.

In an embodiment, each R² is independently selected from D, halo, CN, C₁₋₆ alkyl, C₁₋₆ haloalkyl, C₃₋₄ cycloalkyl, OR^{a2}, C(O)R^{b2}, C(O)NR^{a2}R^{a2}, C(O)OR^{a2}, OC(O)R^{b2}, OC(O)NR^{a2}R^{a2}, NR^{a2}R^{a2}, NR^{a2}C(O)R^{b2}, NR^{a2}C(O)OR^{a2}, NR^{a2}C(O)NR^{a2}R^{a2}, NR^{a2}S(O)₂R^{b2}, NR^{a2}S(O)₂NR^{a2}R^{a2}, S(O)₂R^{b2}, and S(O)₂NR^{a2}R^{a2}, wherein said C₁₋₆ alkyl, C₁₋₆ haloalkyl, and C₃-4 cycloalkyl are each substituted with 0, 1, 2, 3, or 4 substituents independently selected from C₁₋₄ alkyl, C₃₋₇ cycloalkyl, cyclopropyl, oxo, —C(O)C₁₋₄alkyl, —C(O)OC₁₋₄alkyl, —C(O)NH₂, —OH, —F, —Cl, —O—C₁₋₄alkyl and —CN.

In an embodiment, each R² is independently selected from D, halo, CN, C₁₋₄ alkyl, C₁₋₄ haloalkyl, OR^{a2}, and C(O)OR^{a2}, wherein said C₁₋₄ alkyl and C₁₋₄ haloalkyl are each substituted with 0, 1, 2, 3, or 4 substituents independently selected from C₁₋₄ alkyl, C₃₋₇ cycloalkyl, cyclopropyl, oxo, —C(O)C₁₋₄alkyl, —C(O)OC₁₋₄alkyl, —C(O)NH₂, —OH, —F, —Cl, —O—C₁₋₄alkyl and —CN.

In an embodiment, each R² is independently selected from halo, CN, C₁₋₄ alkyl, C₁₋₄ haloalkyl, OH, C₁₋₃ alkoxy, C₁₋₃ haloalkoxy, amino, C₁₋₃ alkylamino, di(C₁₋₃ alkyl)amino, cyano-C₁₋₄ alkyl, HO—C₁₋₄ alkyl, C₁₋₃ alkoxy-C₁₋₄ alkyl, and C₃₋₄ cycloalkyl, wherein each R² is as defined in any of the embodiments described herein. In an embodiment, each R² is independently selected from H, halo, OH, C₁₋₃ alkoxy, and C₁₋₃ haloalkoxy. In an embodiment, each R² is independently selected from OH, C₁₋₃ alkoxy, and C₁₋₃ haloalkoxy. In an embodiment, each R² is independently selected from halo, CN, C₁₋₃ alkyl, and C₁₋₃ haloalkyl. In an embodiment, each R² is independently selected from halo, —CN, —Me, —OMe and —OH. In an embodiment, each R² is independently selected from —F and —Me. In an embodiment, each R² is independently —F. In an embodiment, each R² is independently —Me,

As generally defined herein, each R^{a2} is independently selected from H, C₁₋₆ alkyl, C₁₋₆ hydroxyalkyl, C₃₋₉ cycloalkyl and C₂₋₆ heteroalkyl, or, when possible, two instances of R^{a2} and the atom to which they are attached are taken together to form a 4-7 member heterocycle substituted with 0, 1, 2, 3, or 4 substituents independently selected from —D, halo, —OH, —C₁₋₄ alkyl and —OC₁₋₄ alkyl.

In an embodiment, each R^{a2} is independently selected from H, C₁₋₆ alkyl, C₁₋₆ hydroxyalkyl, C₃₋₉ cycloalkyl and C₂₋₆ heteroalkyl, or, when possible, two instances of R^{a2} and the atom to which they are attached are taken together to form a saturated 3-7-membered heterocycle.

In an embodiment, each R^{a2} is independently selected from H and C₁₋₆ alkyl.

In an embodiment, each R^{a2} is independently selected from H, —Me, —Et, -iPr, cyclopropyl, cyclobutyl, cyclopentyl, cyclohexyl, alkyl and C₂₋₆ heteroalkyl, or, when possible, two instances of R^{a2} and the atom to which they are attached are taken together to form a saturated 3-6-membered heterocycle.

In an embodiment, each R^{a2} is independently selected from H and —Me.

As generally defined herein, each R^{b2} is independently selected from C₁₋₆ alkyl, C₁₋₆ hydroxyalkyl, C₃₋₉ cycloalkyl and C₂₋₆ heteroalkyl.

In an embodiment, each R^{b2} is independently selected from C₁₋₆ alkyl and C₃₋₉ cycloalkyl.

In an embodiment, each R^{b2} is independently selected from C₁₋₆ alkyl and C₃₋₆ cycloalkyl.

In an embodiment, each R^{b2} is independently selected from —Me, —Et, -iPr, cyclopropyl, cyclobutyl, cyclopentyl, cyclohexyl, alkyl and C₂₋₆ heteroalkyl, or, when possible, two instances of R^{a2} and the atom to which they are attached are taken together to form a saturated 3-6-membered heterocycle.

In an embodiment, each R^{b2} is independently selected from —Me, —Et and ′Pr,

In an embodiment, each R^{b2} is independently —Me.

As generally defined herein, L is a covalent bond or a bivalent, saturated or unsaturated, straight or branched C₁₋₅o hydrocarbon chain, wherein 0-6 methylene units of L are independently replaced by —Cy—, —CH(R)—, —C(R)₂—, —O—, —NR—, —S—, —OC(═O)—, —C(═O)O—, —C(═O)—, —S(═O)—, —S(═O)₂—, —NRS(═O)₂—, —S(═O)₂NR—, —NRC(═O)—, —C(═O)NR—, —OC(═O)NR— or —NRC(═O)O—, wherein each —Cy— and —R are as defined in any of the embodiments described herein.

In an embodiment, L is —Z¹—Z²-Z³—Z⁴—Z⁵—wherein:
Z¹ is a bond, C₁₋₆ alkylene, —C(O)NR—, —NR′(CO)—, —S(O)₂NR—, —NR'S(O)₂—, —(O—C₁₋₆ alkylene)ₐ—, —(C₁₋₆ alkylene-O)ₐ—, phenylene, 5-6 membered monocyclic heteroarylene having 1-4 heteroatoms independently selected from nitrogen, oxygen, and sulfur, or 4-11 membered heterocyclylene having 1-2 heteroatoms independently selected from nitrogen, oxygen, and sulfur, where each ring is substituted with 0, 1 or 2 substituents independently selected from C₁₋₆alkyl, C₁₋₆ alkoxy, halo, C₁₋₆ haloalkyl, and C₁₋₆ haloalkoxy;
Z² is a bond, C₁₋₆ alkylene, C₂₋₆ alkynylene, —C(O)—, —C(O)N(R^{cc})—, —NR′(CO)—, —(O—C₁₋₆ alkylene)_{b}-, —(C₁₋₈ alkylene-O)_{b}—, —(O—C₁₋₈ alkylene)_{b}, —C₃₋₁₁ cycloalkylene, 4-11 membered heterocyclylene having 1-2 heteroatoms independently selected from nitrogen, oxygen, and sulfur, where each ring is substituted with 0, 1 or 2 substituents independently selected from C₁₋₆ alkyl, C₁₋₆alkoxy, halo, C₁₋₆haloalkyl, and C₁₋₆haloalkoxy;
Z³ is a bond, C₁₋₆ alkylene, C₂₋₆ alkynylene, —C(O)NR—, —NR′(CO)—, —O—, —NR″—, —(O—C₁₋₈ alkylene)_{c}-, —(C₁₋₈ alkylene-O)_{c}—, C₃₋₁₁ cycloalkylene, C₅₋₁₁ spiro cycloalkylene, phenylene, 5-6 membered monocyclic heteroarylene having 1-4 heteroatoms independently selected from nitrogen, oxygen, and sulfur, 4-7 membered monocyclic heterocyclylene having 1-2 heteroatoms independently selected from nitrogen, oxygen, and sulfur, a 5-11 membered spiro heterocyclylene having 1-2 heteroatoms independently selected from nitrogen, oxygen, and sulfur, an 4-10 membered bicyclic heterocyclylene having 1-2 heteroatoms independently selected from nitrogen, oxygen, and sulfur, a 5-10 membered bridged bicyclic saturated or partially unsaturated heterocyclylene having 1-2 heteroatoms independently selected from nitrogen, oxygen, and sulfur, where each ring is substituted with 0, 1 or 2 substituents independently selected from alkyl, alkoxy, halo, haloalkyl, and haloalkoxy;
Z⁴ is a bond, alkylene, alkynylene, -(alkylene-NR″)—, —O—, —C(O)—, —NR″—, —(O-alkylene)ₐ—, -(alkylene-O)ₐ—, C₃₋₁₁ cycloalkylene, C₅₋₁₁ spiro cycloalkylene, phenylene, a 4-7 membered monocyclic heterocyclylene having 1-2 heteroatoms independently selected from nitrogen, oxygen, and sulfur, a 5-11 membered spiro heterocyclylene having 1-2 heteroatoms independently selected from nitrogen, oxygen, and sulfur, an 4-10 membered bicyclic heterocyclylene having 1-2 heteroatoms independently selected from nitrogen, oxygen, and sulfur, a 5-10 membered bridged bicyclic saturated or partially unsaturated heterocyclylene having 1-2 heteroatoms independently selected from nitrogen, oxygen, and sulfur, a 5-6 membered heteroarylene having 1-4 heteroatoms independently selected from nitrogen, oxygen, and sulfur, or an 8-10 membered bicyclic heteroarylene having 1-5 heteroatoms independently selected from nitrogen, oxygen, or sulfur, where each ring is substituted with 0, 1 or 2 substituents independently selected from alkyl, alkoxy, halo, haloalkyl, and haloalkoxy;
Z⁵ is a bond, -alkylene, —NR″—, —O—, —C(O)—, —S(O)₂—, —NR′(CO)—, —C(O)NR—, phenylene, a 4-7 membered monocyclic heterocyclylene having 1-2 heteroatoms independently selected from nitrogen, oxygen, and sulfur, a 5-11 membered spiro heterocyclylene having 1-2 heteroatoms independently selected from nitrogen, oxygen, and sulfur, an 4-10 membered bicyclic heterocyclylene having 1-2 heteroatoms independently selected from nitrogen, oxygen, and sulfur, a 5-10 membered bridged bicyclic saturated or partially unsaturated heterocyclylene having 1-2 heteroatoms independently selected from nitrogen, oxygen, and sulfur, a 5-6 membered heteroarylene having 1-4 heteroatoms independently selected from nitrogen, oxygen, and sulfur, or an 8-10 membered bicyclic heteroarylene having 1-5 heteroatoms independently selected from nitrogen, oxygen, or sulfur, where each ring is substituted with 0, 1 or 2 substituents independently selected from alkyl, alkoxy, halo, haloalkyl, and haloalkoxy;
each R, R′ and R″ is independently selected from H and C₁₋₆ alkyl;
each a, b, c, and d is independently an integer selected from 1, 2, 3, 4, 5 and 6;
and each alkylene is substituted with 0, 1, 2, 3 or 4 substituents where 0, 1, 2, or 3 substituents are independently selected from fluoro and deuterium, and 0 or 1 substituents are carboxy;
provided that at least one of —Z¹—Z²-Z³—Z⁴—Z⁵—is not a bond.

In an embodiment, L is selected from the group consisting of:

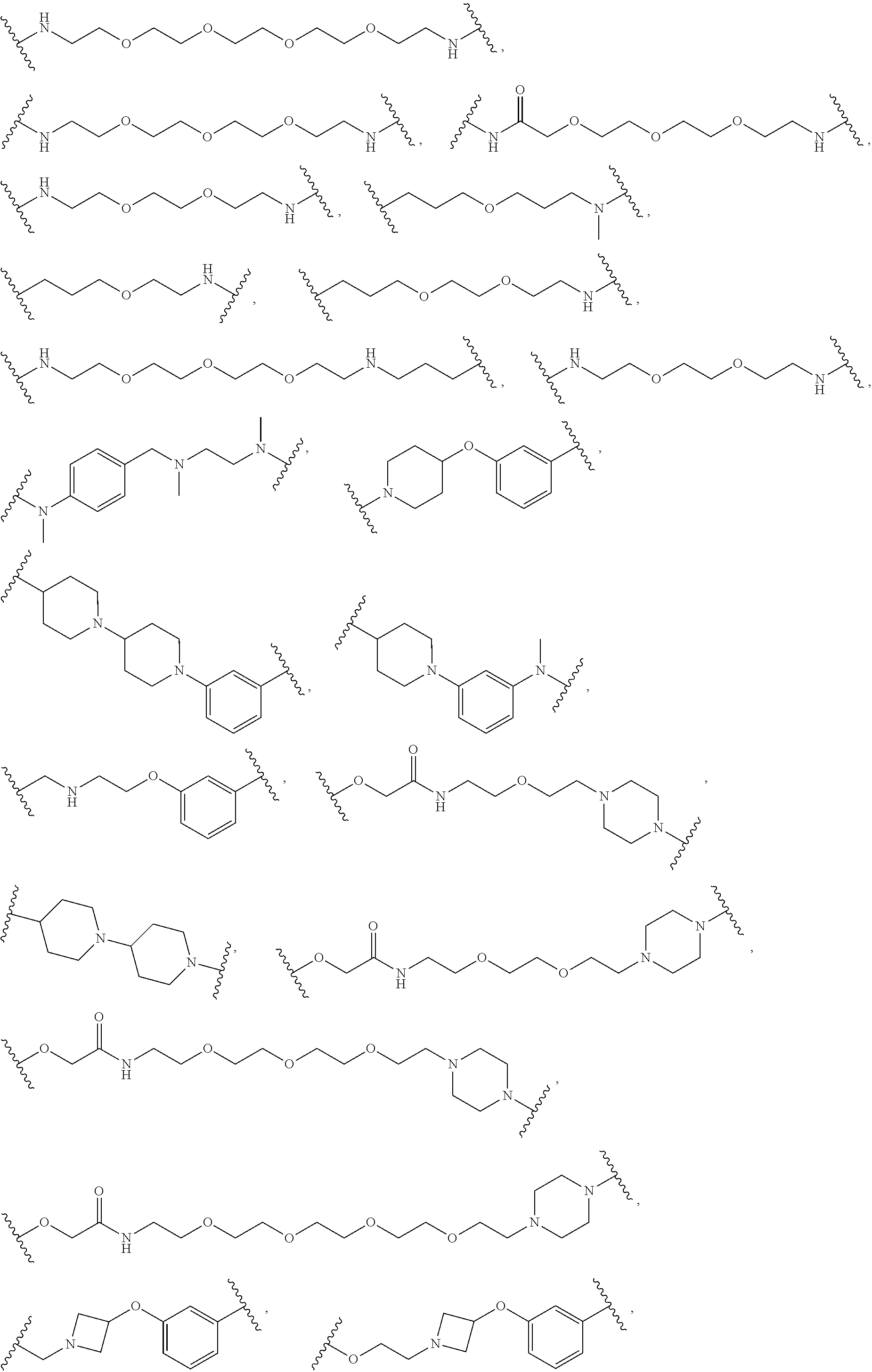

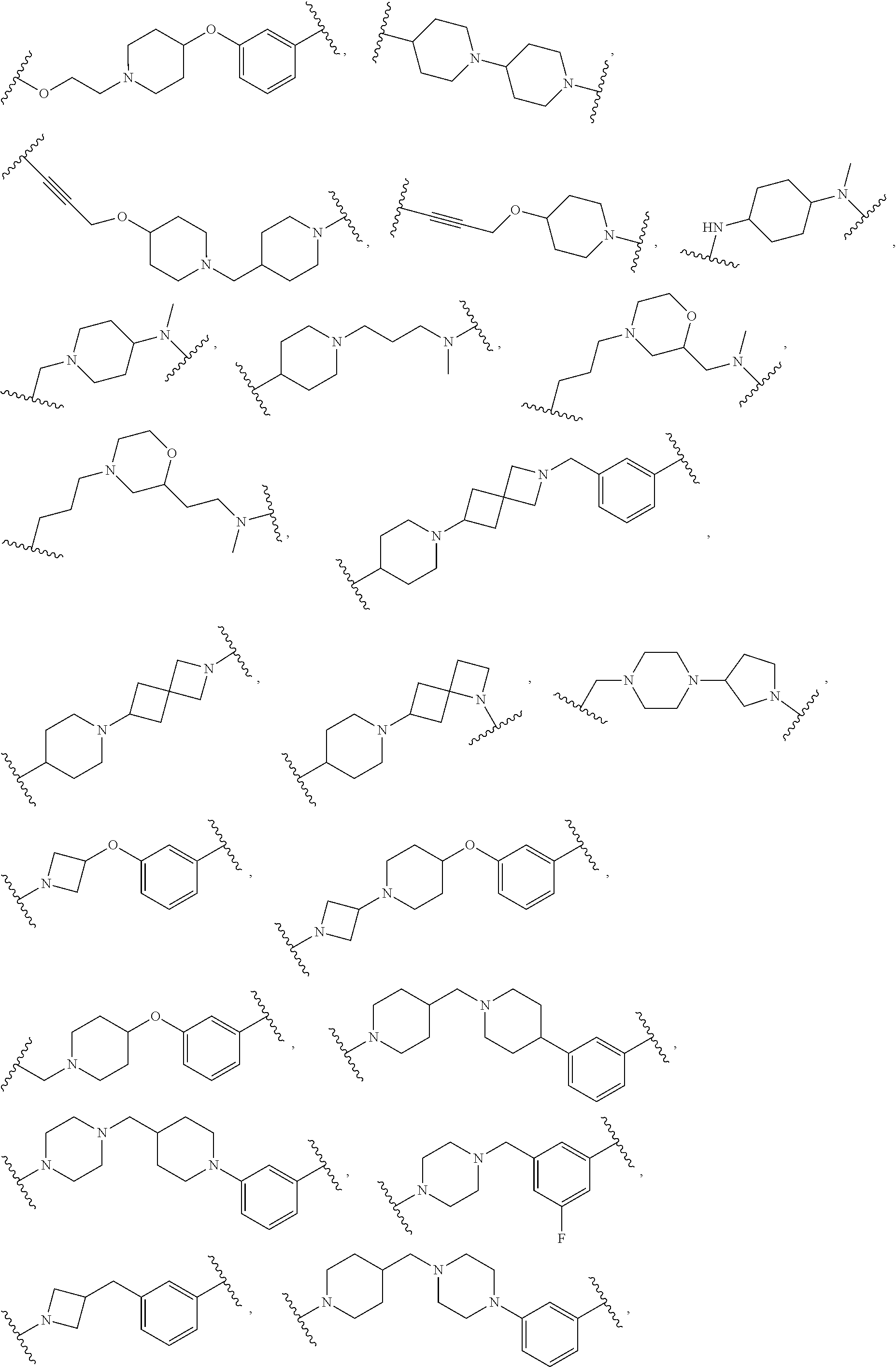

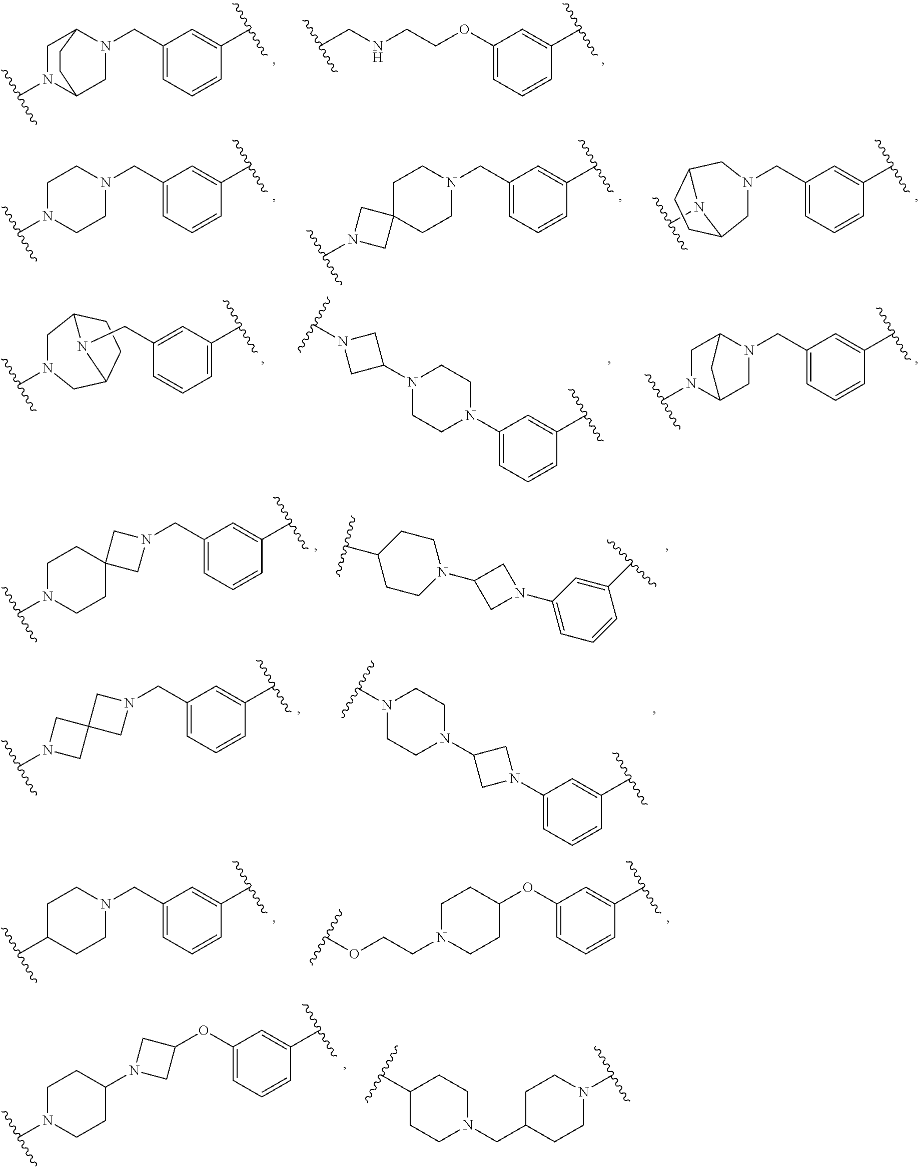

wherein the left attachment point connects to LBM and the right side connects to X of Formula A-I or the —S(O)₂— group of Formula I.

In an embodiment, L is selected from List L, wherein either end of L can be attached to X of Formula A-I or the —S(O)₂— group of Formula I.

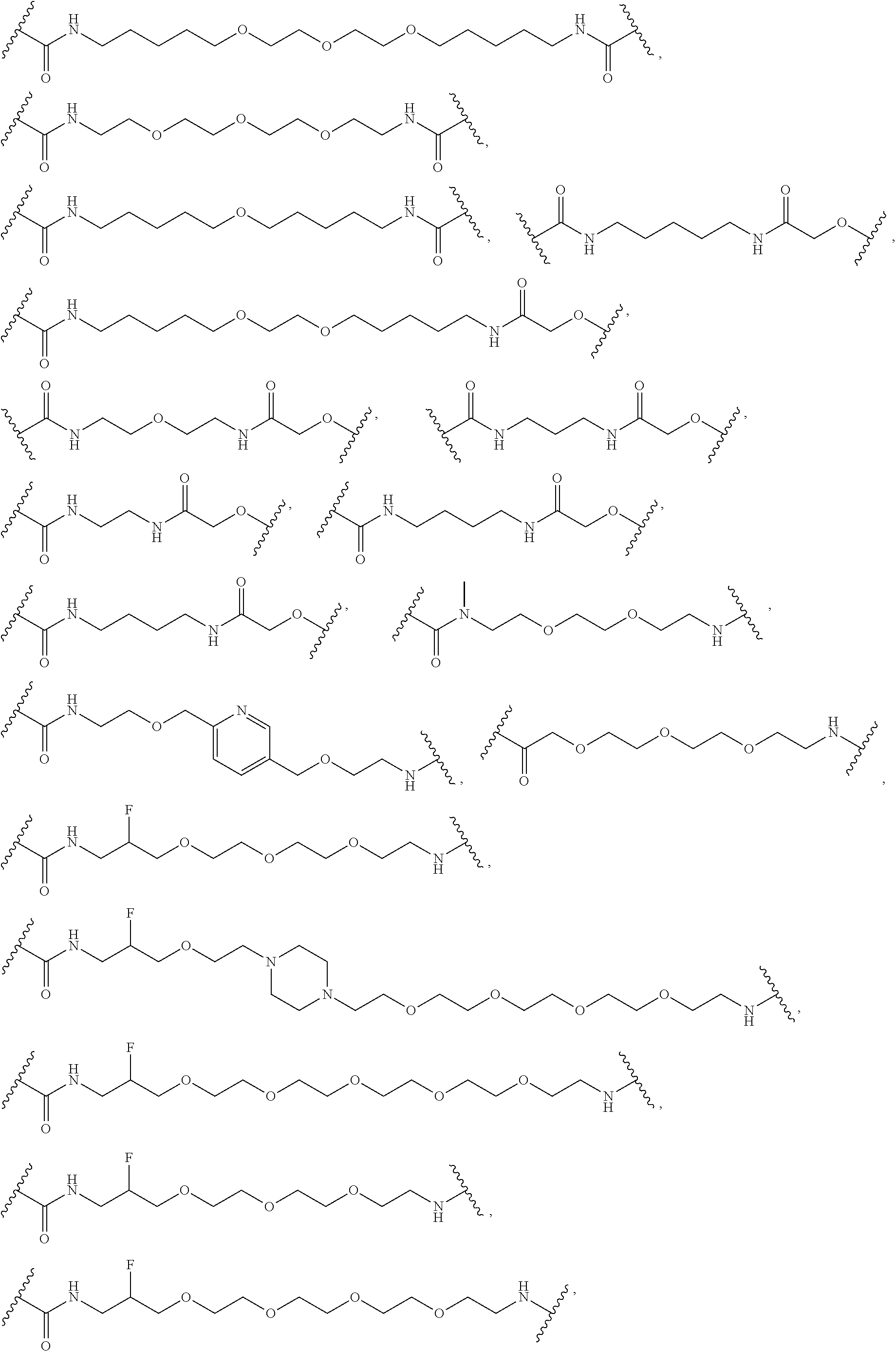

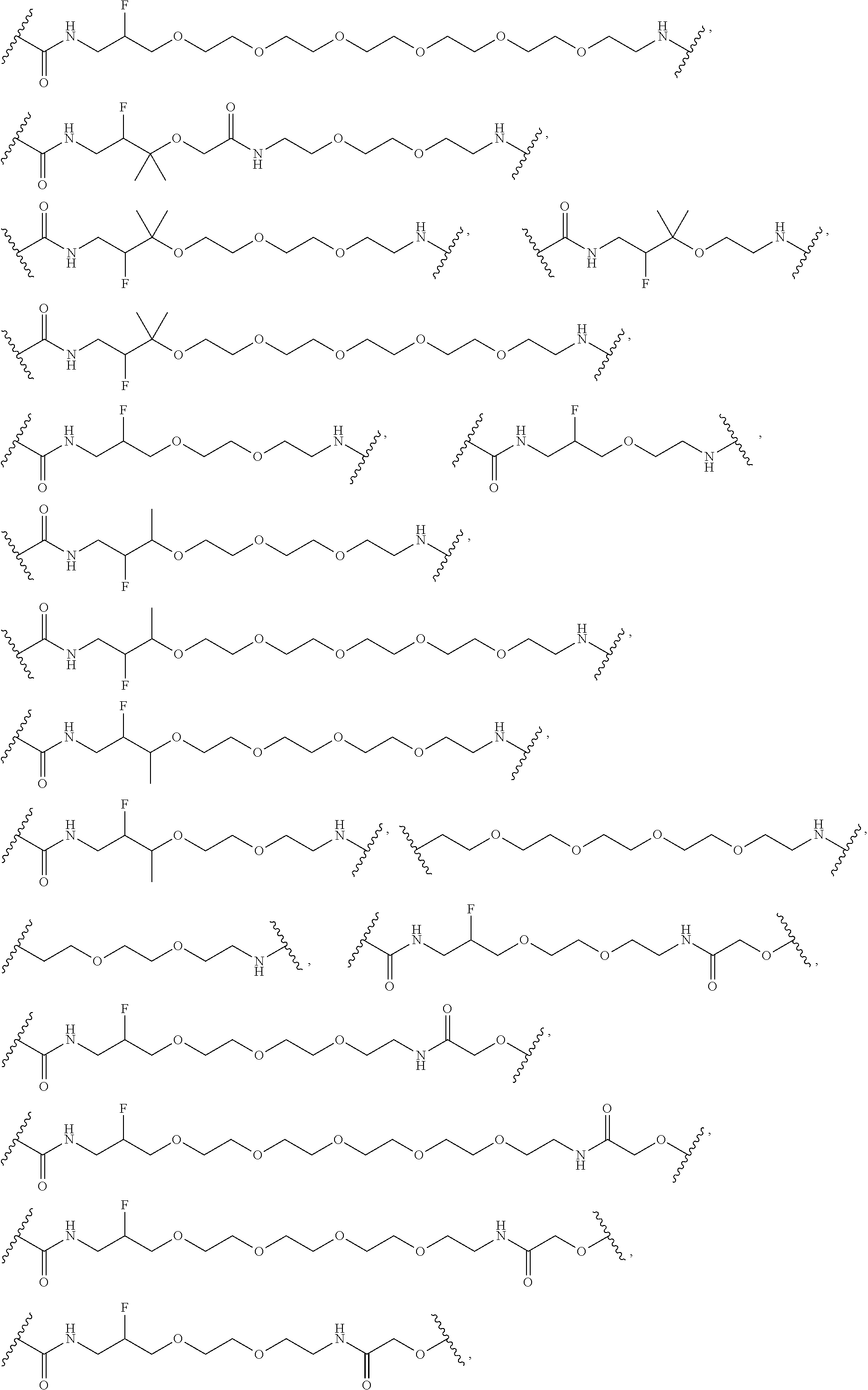

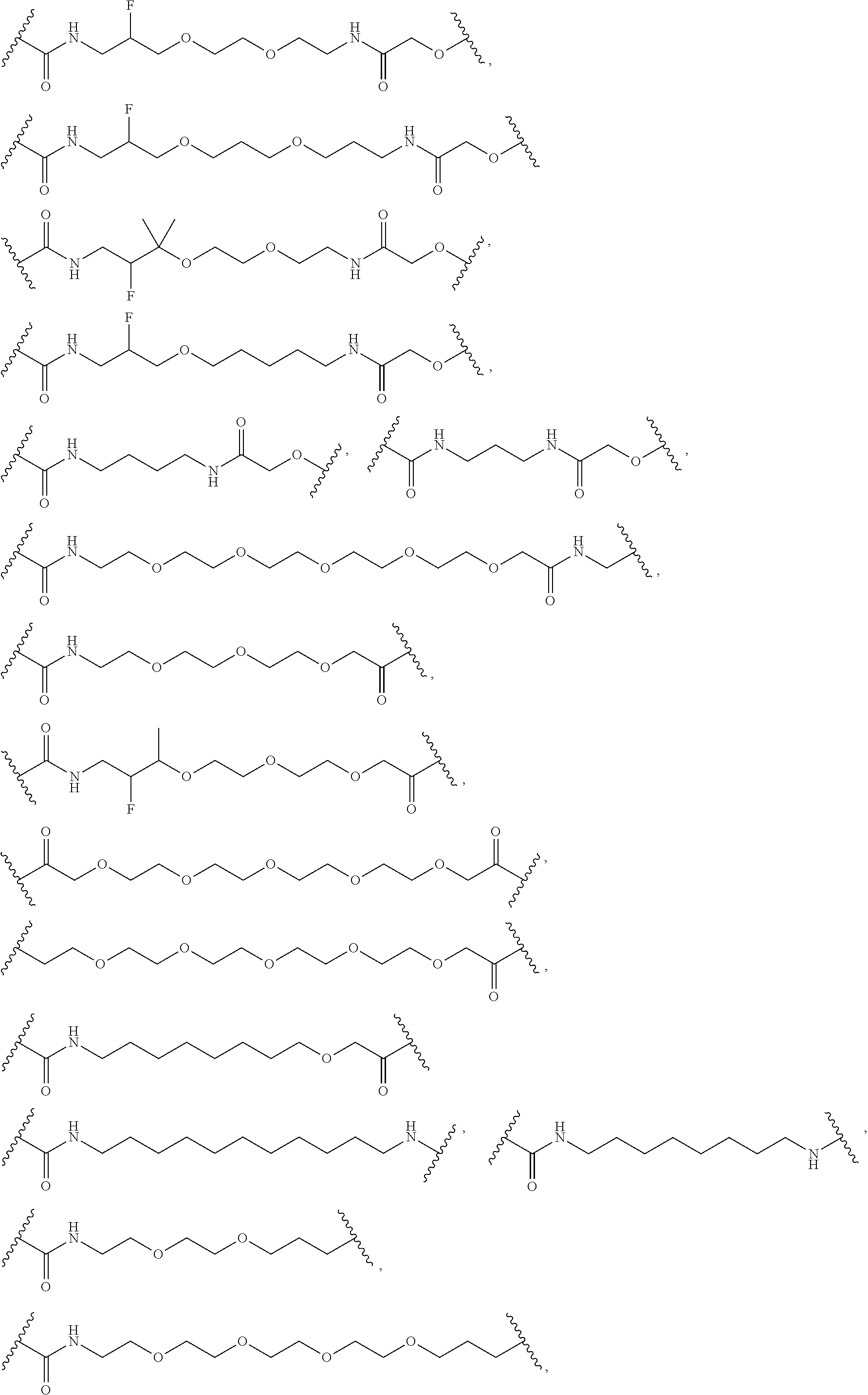

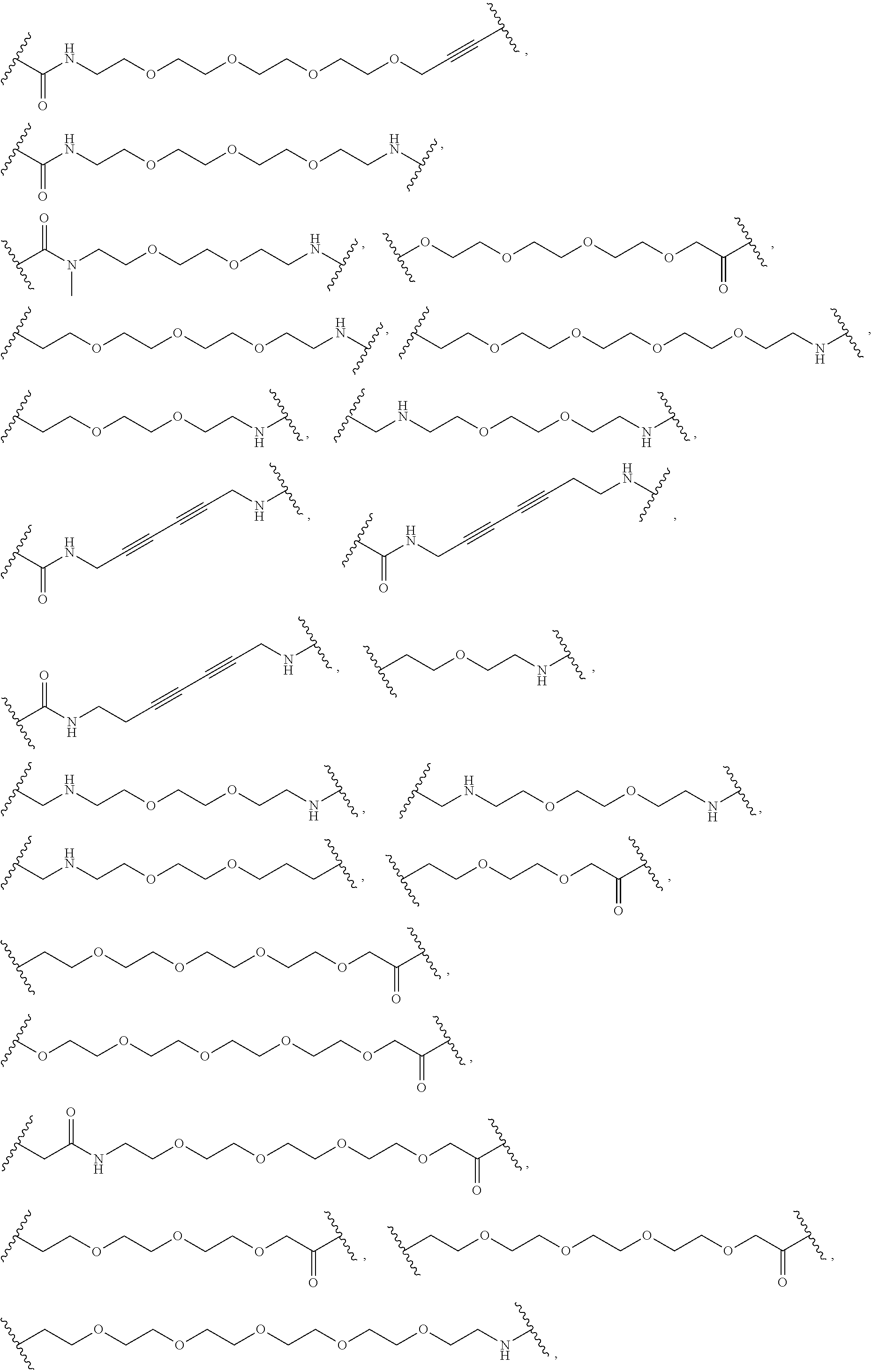

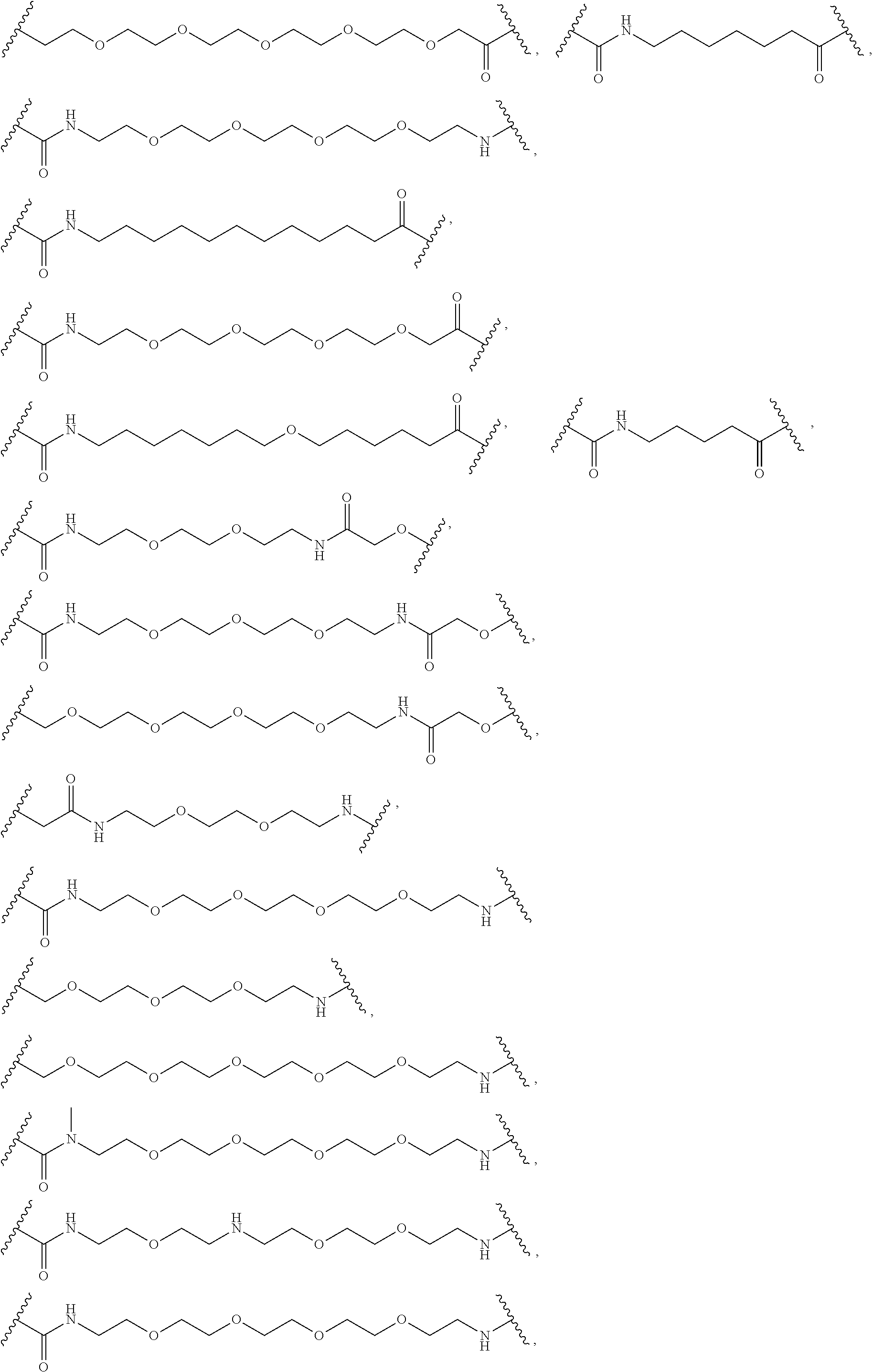

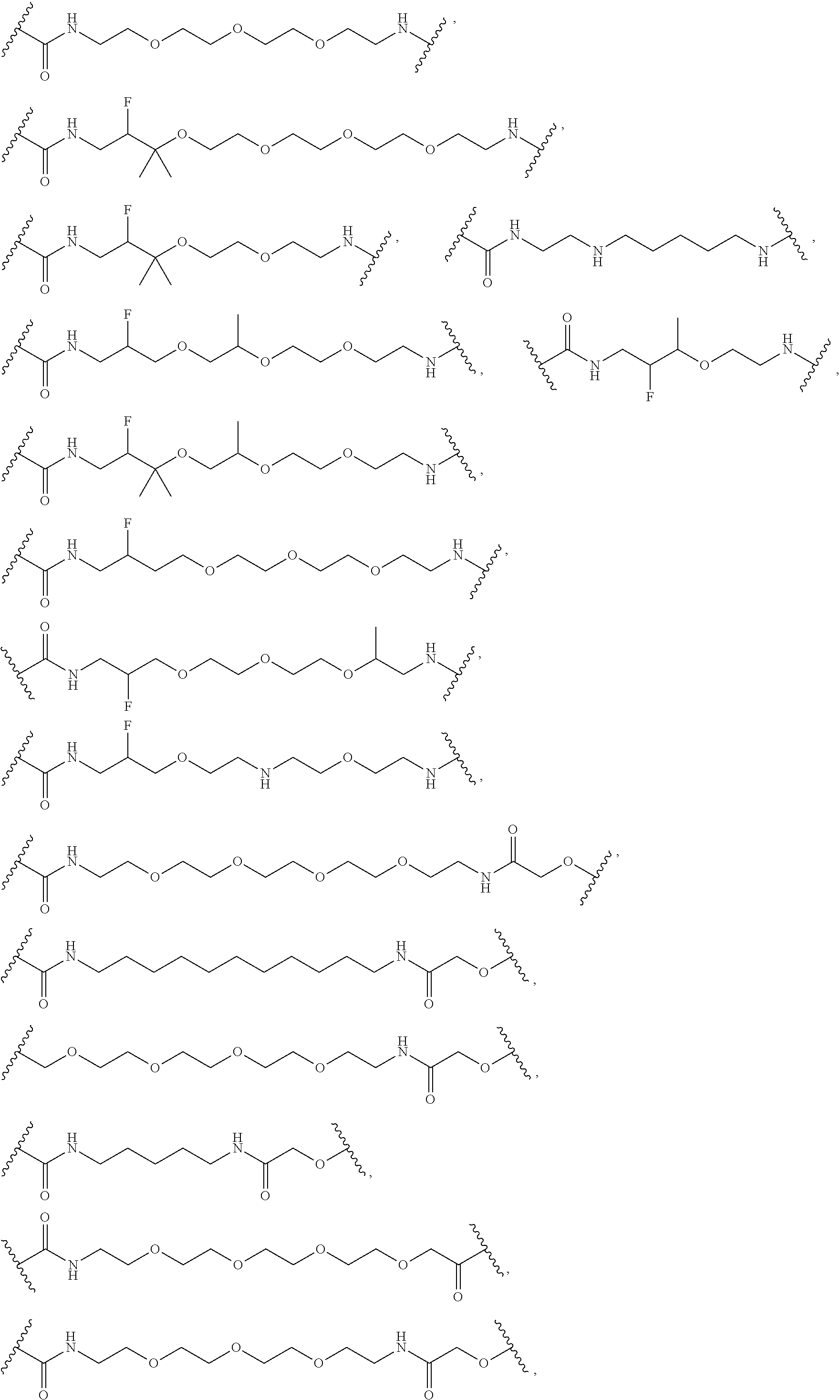

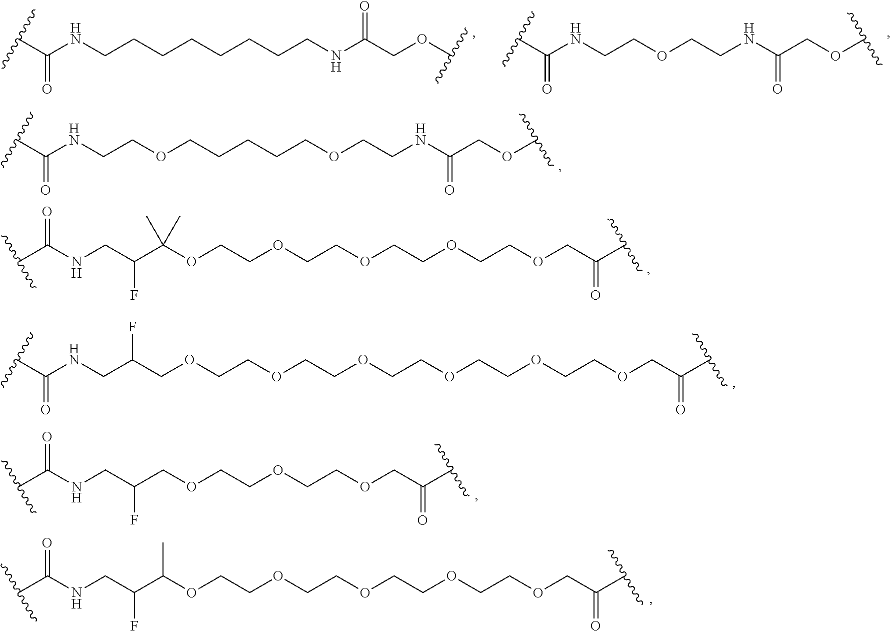

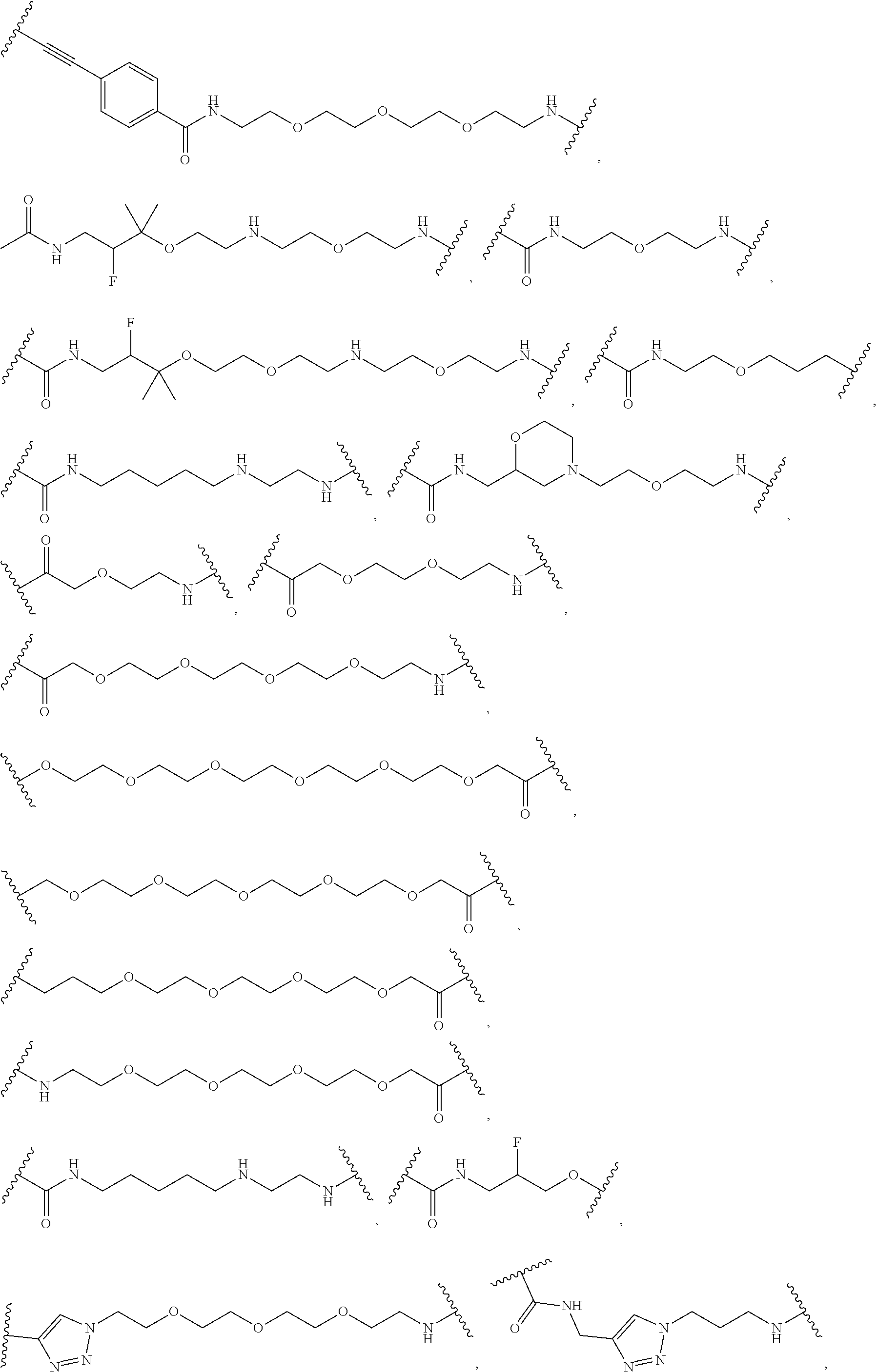

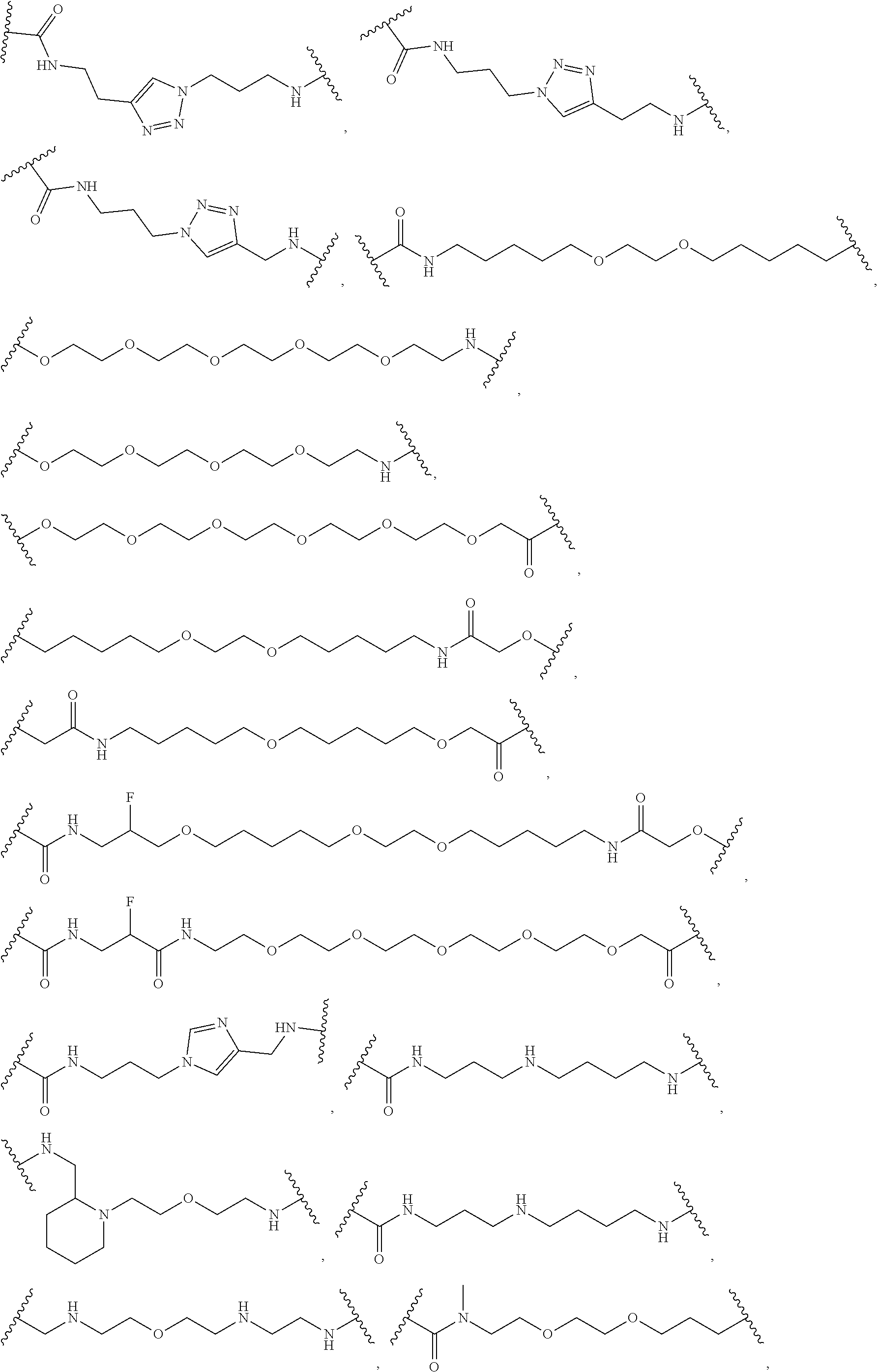

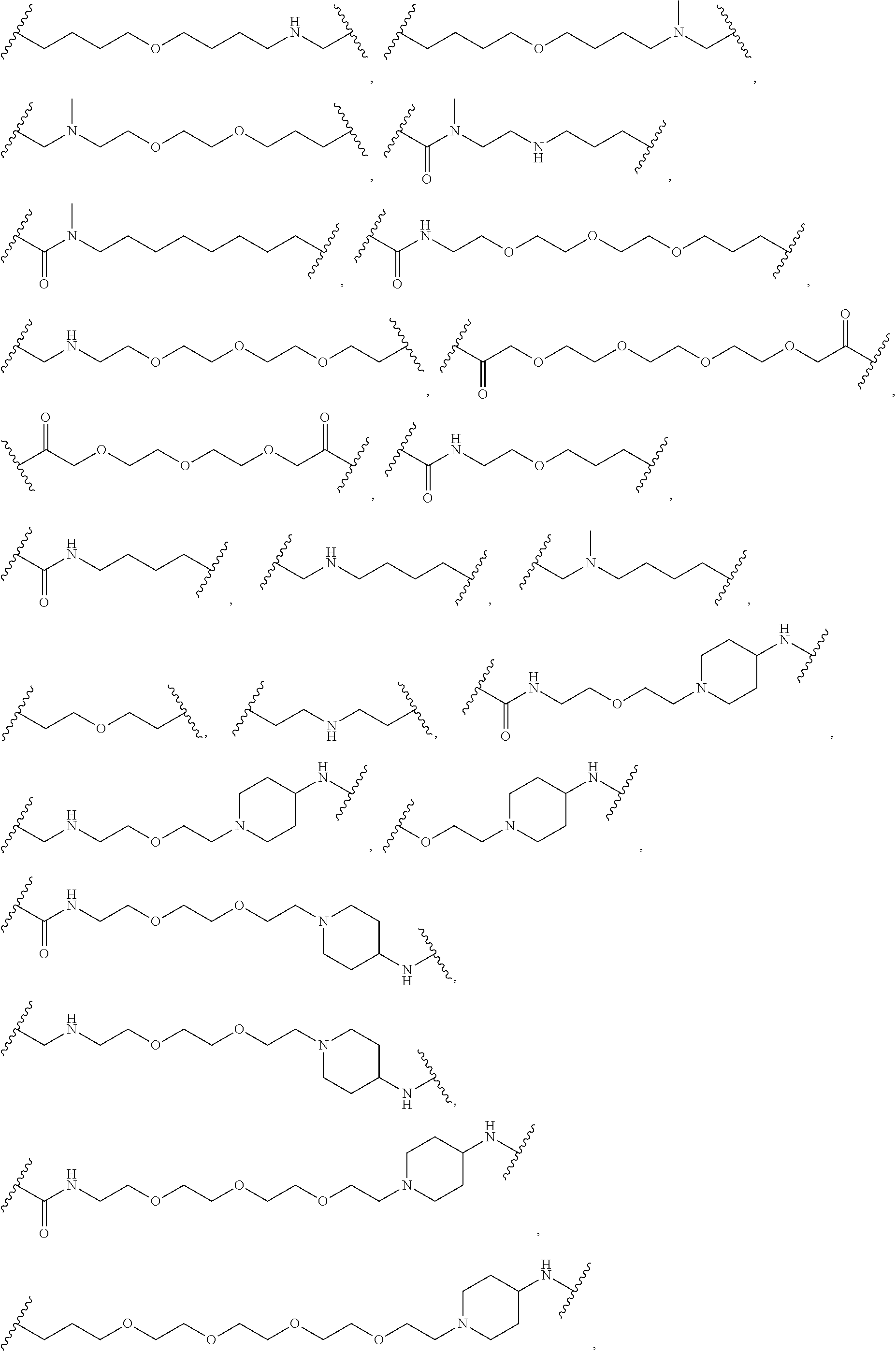

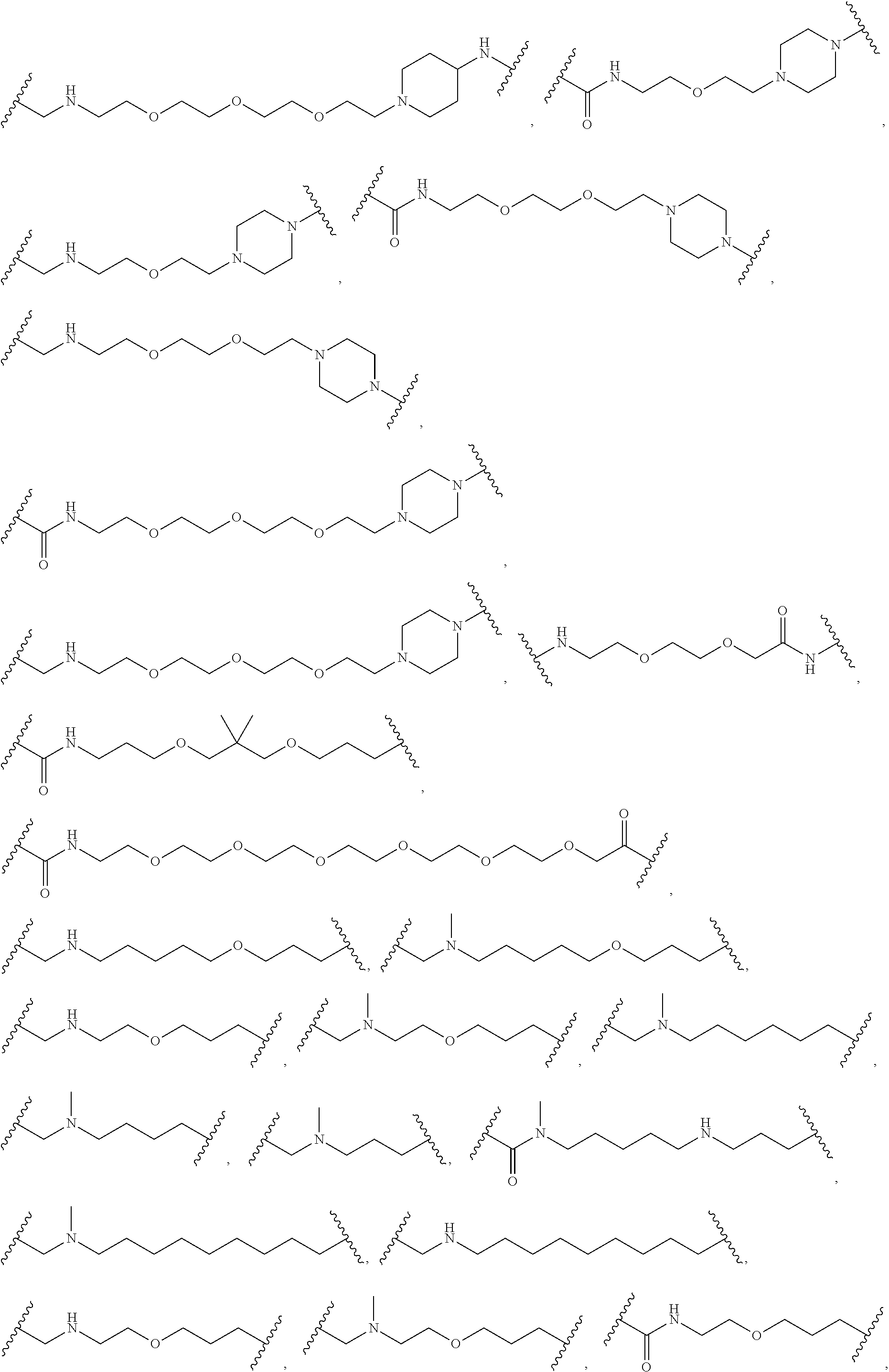

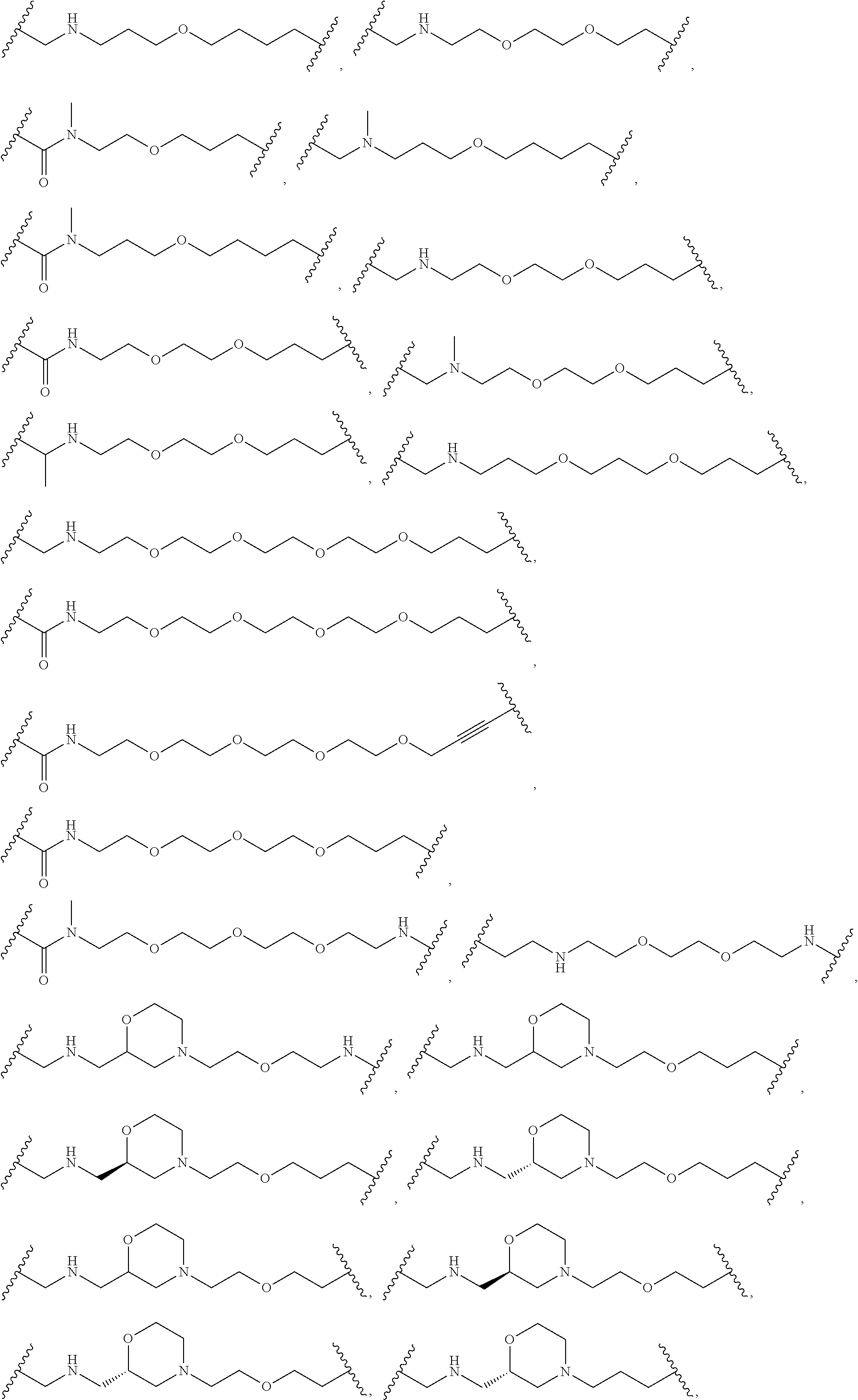

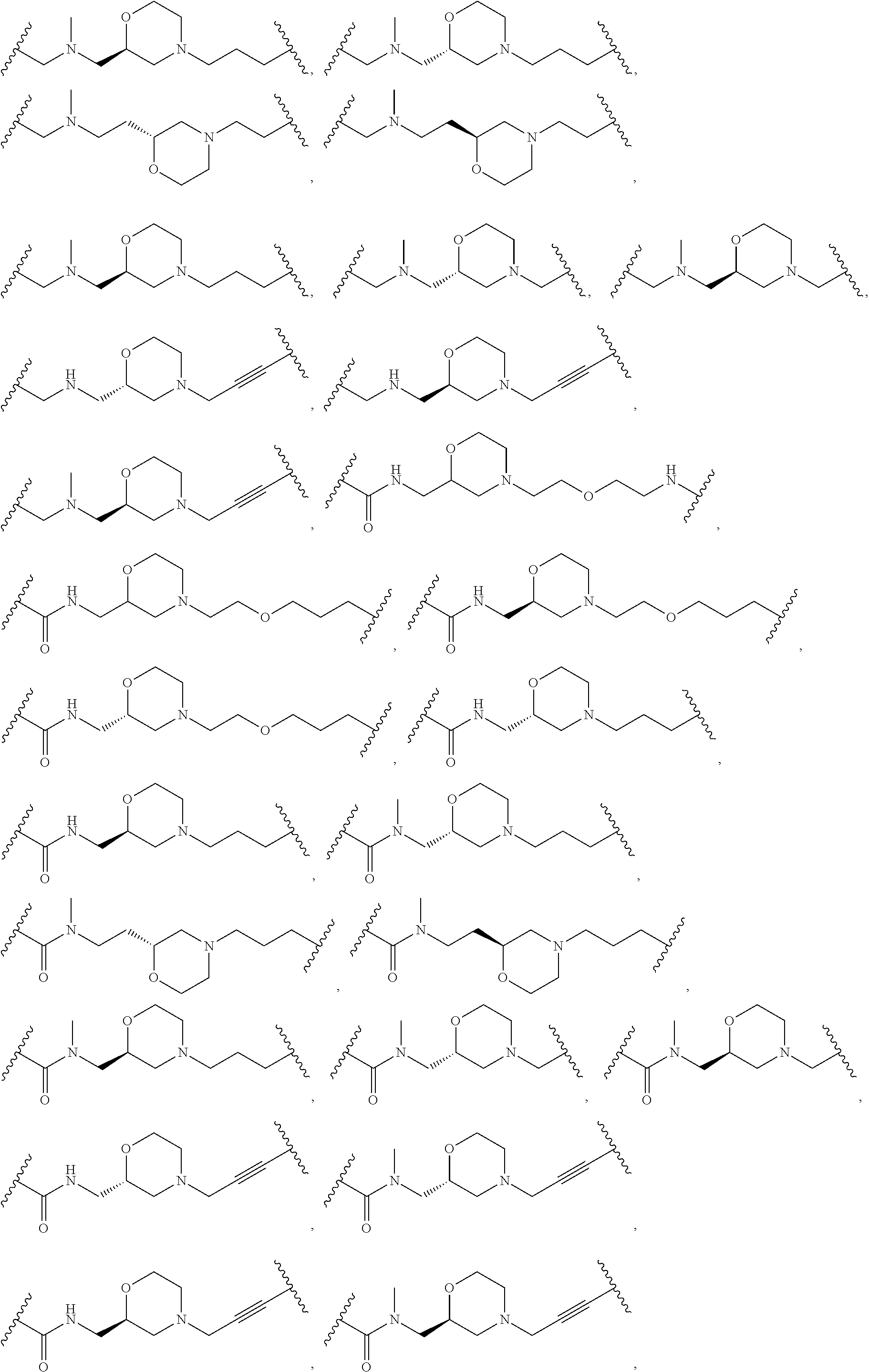

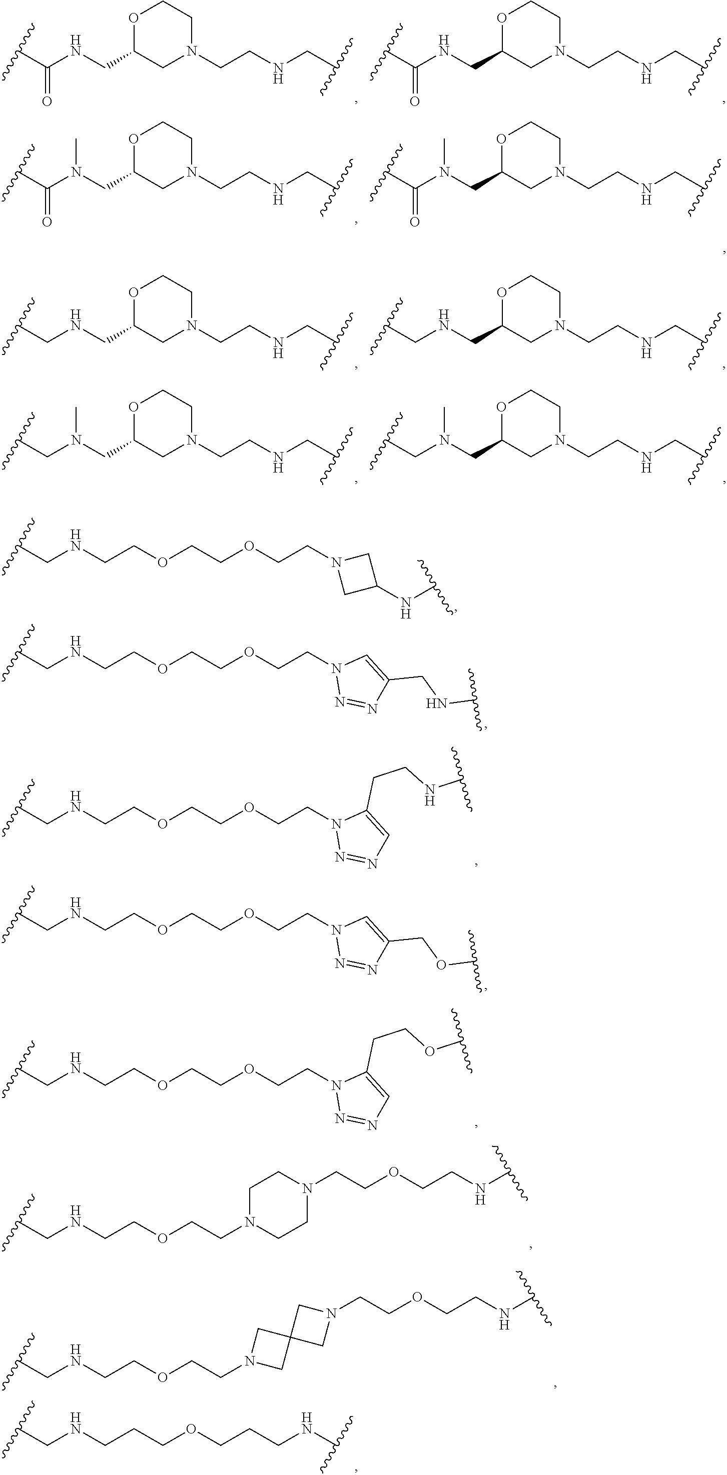

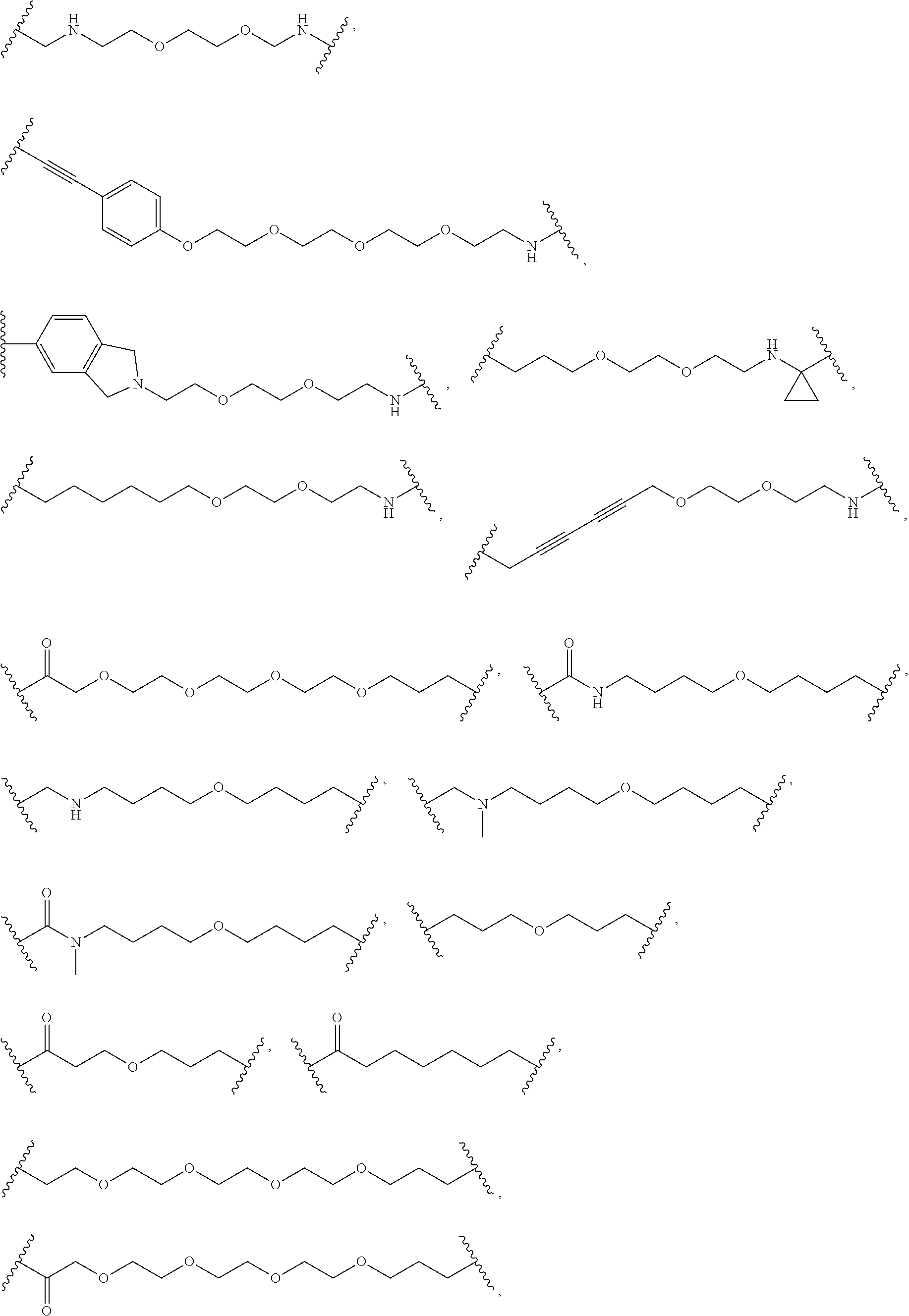

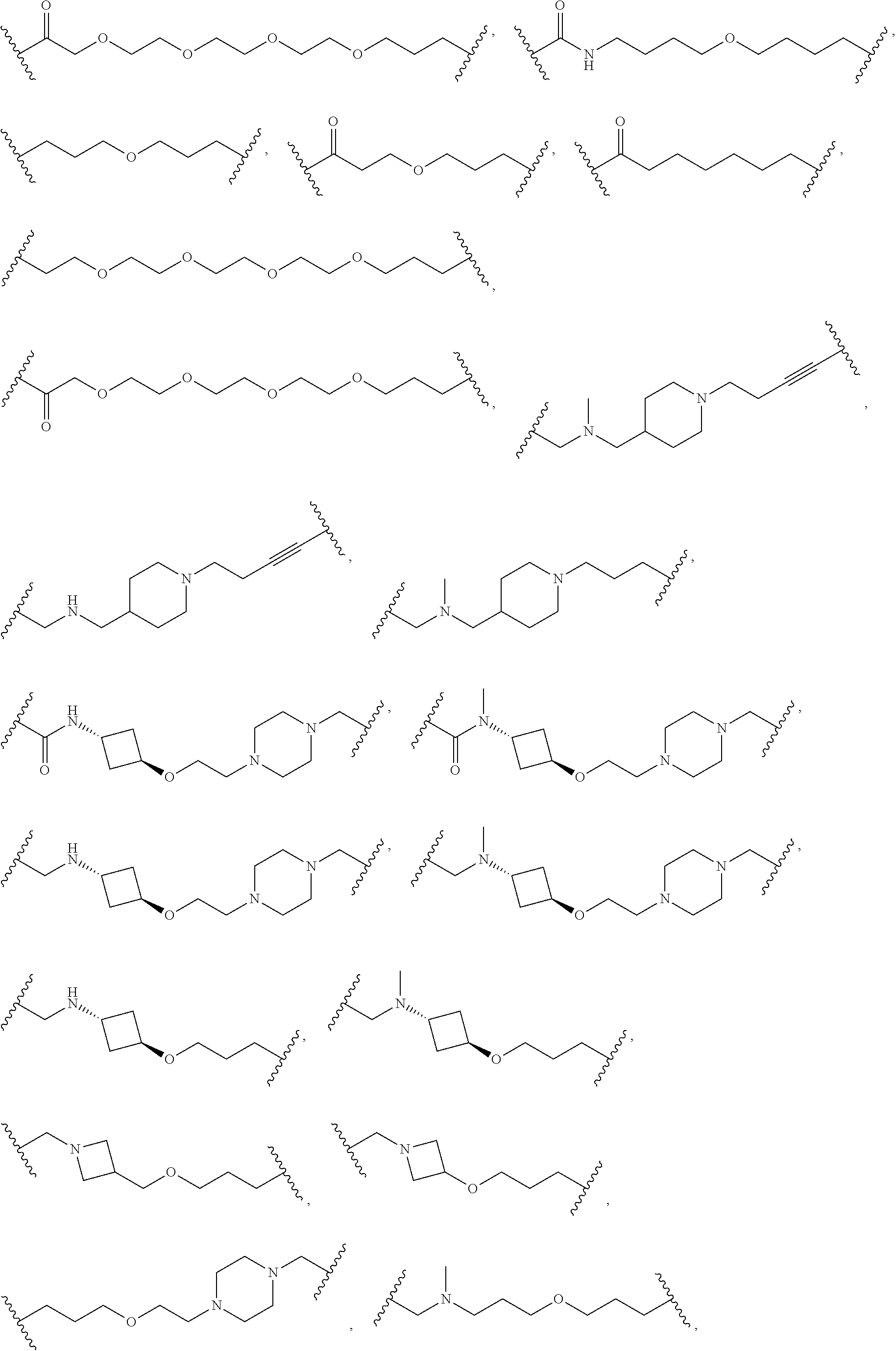

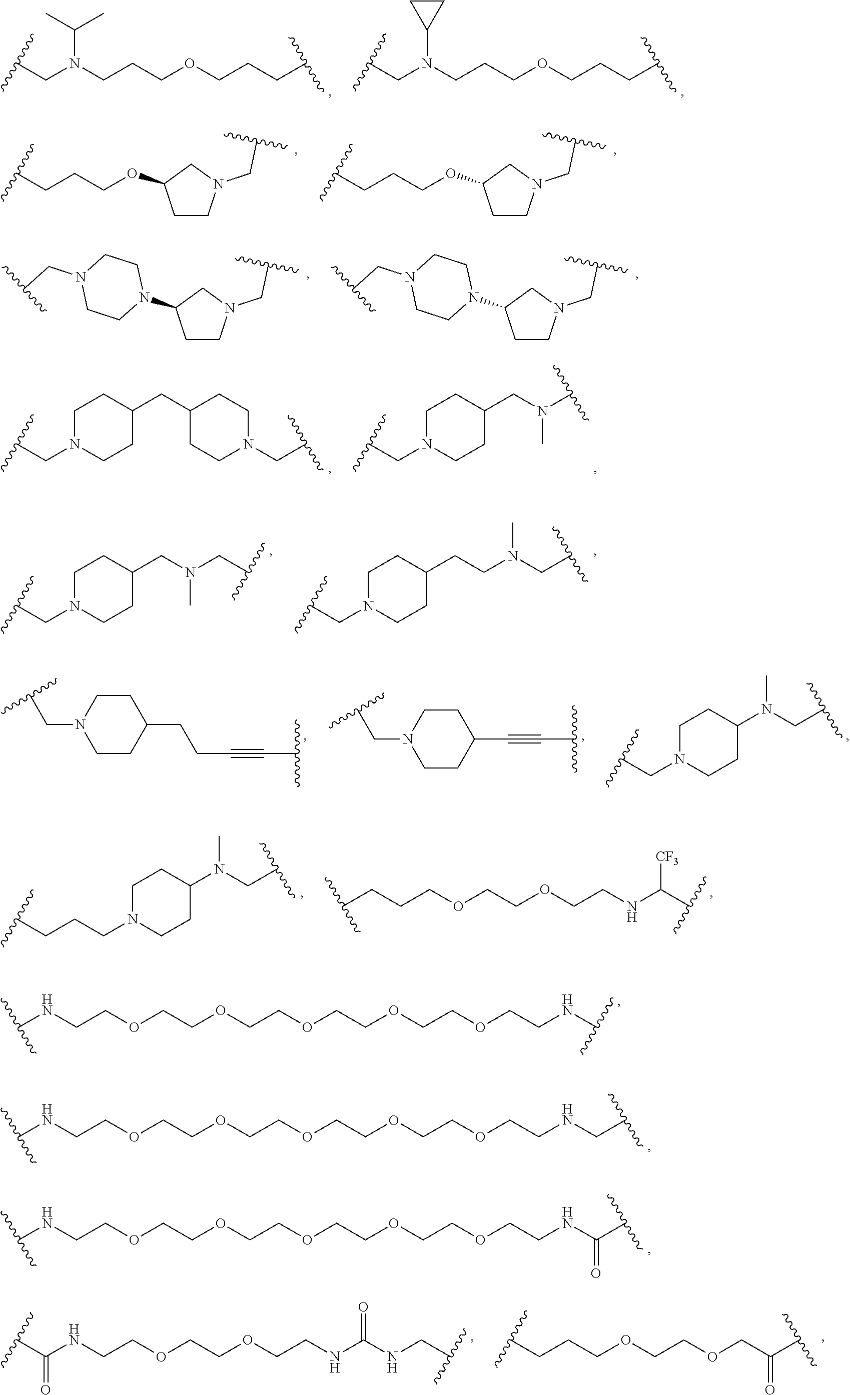

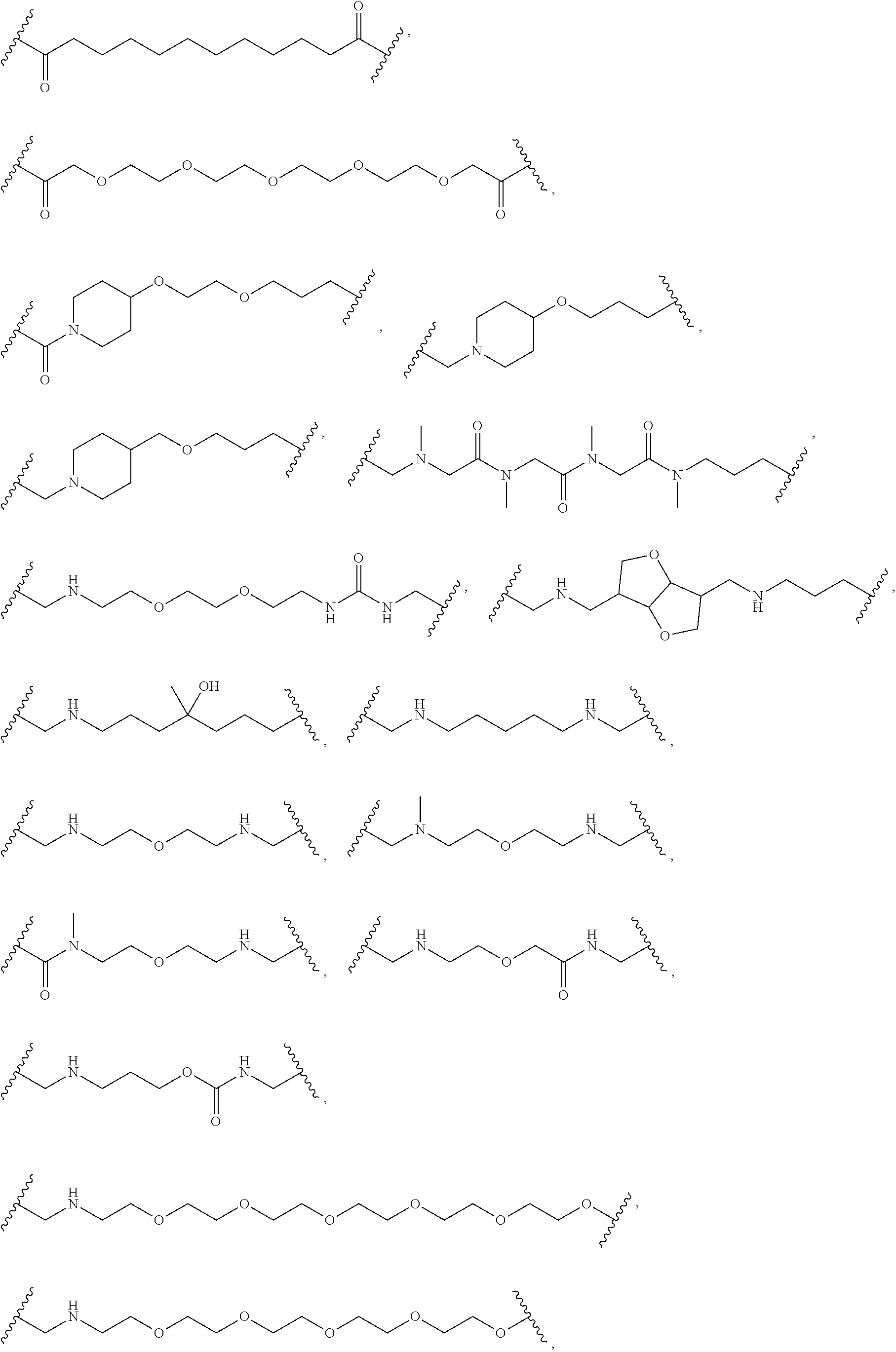

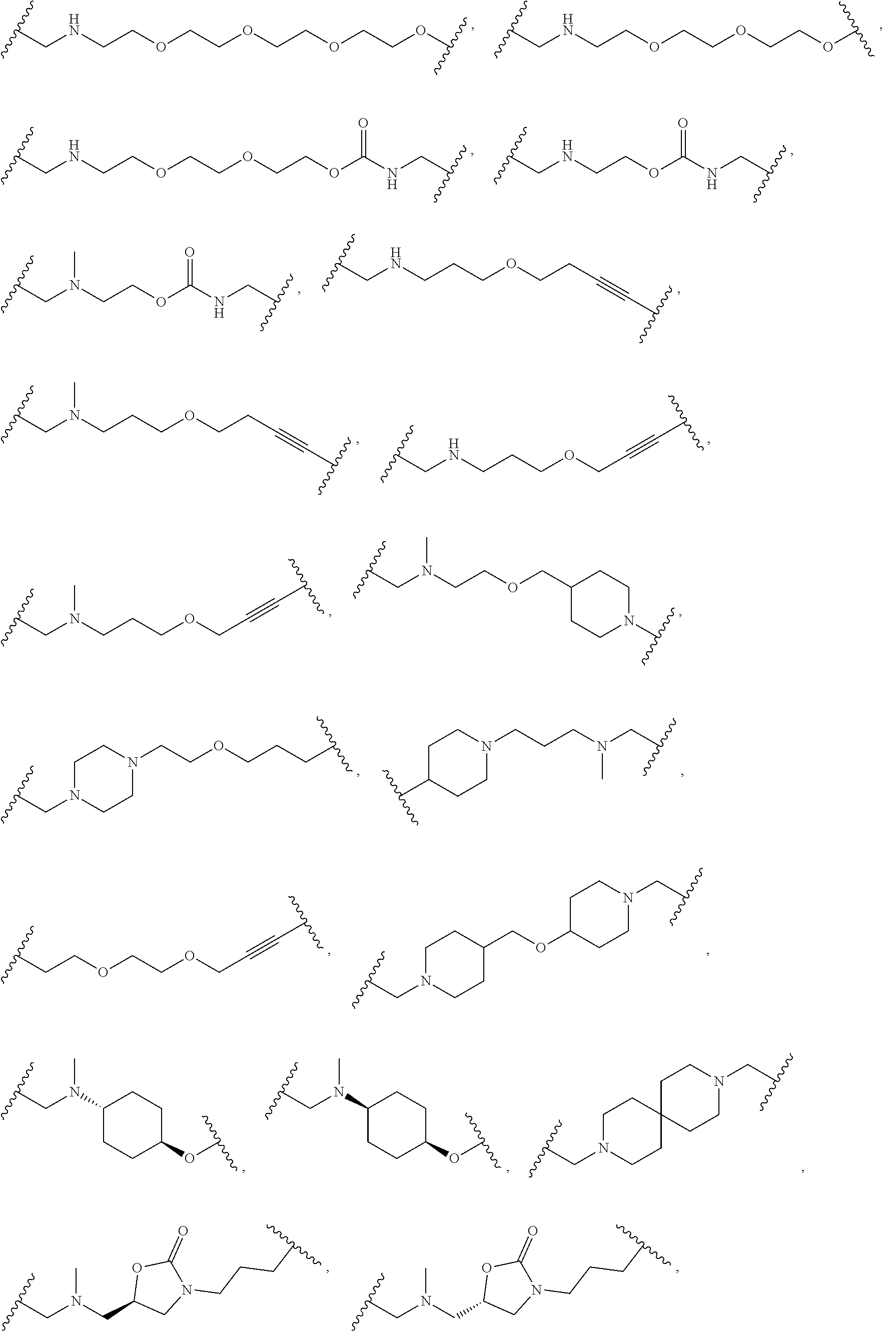

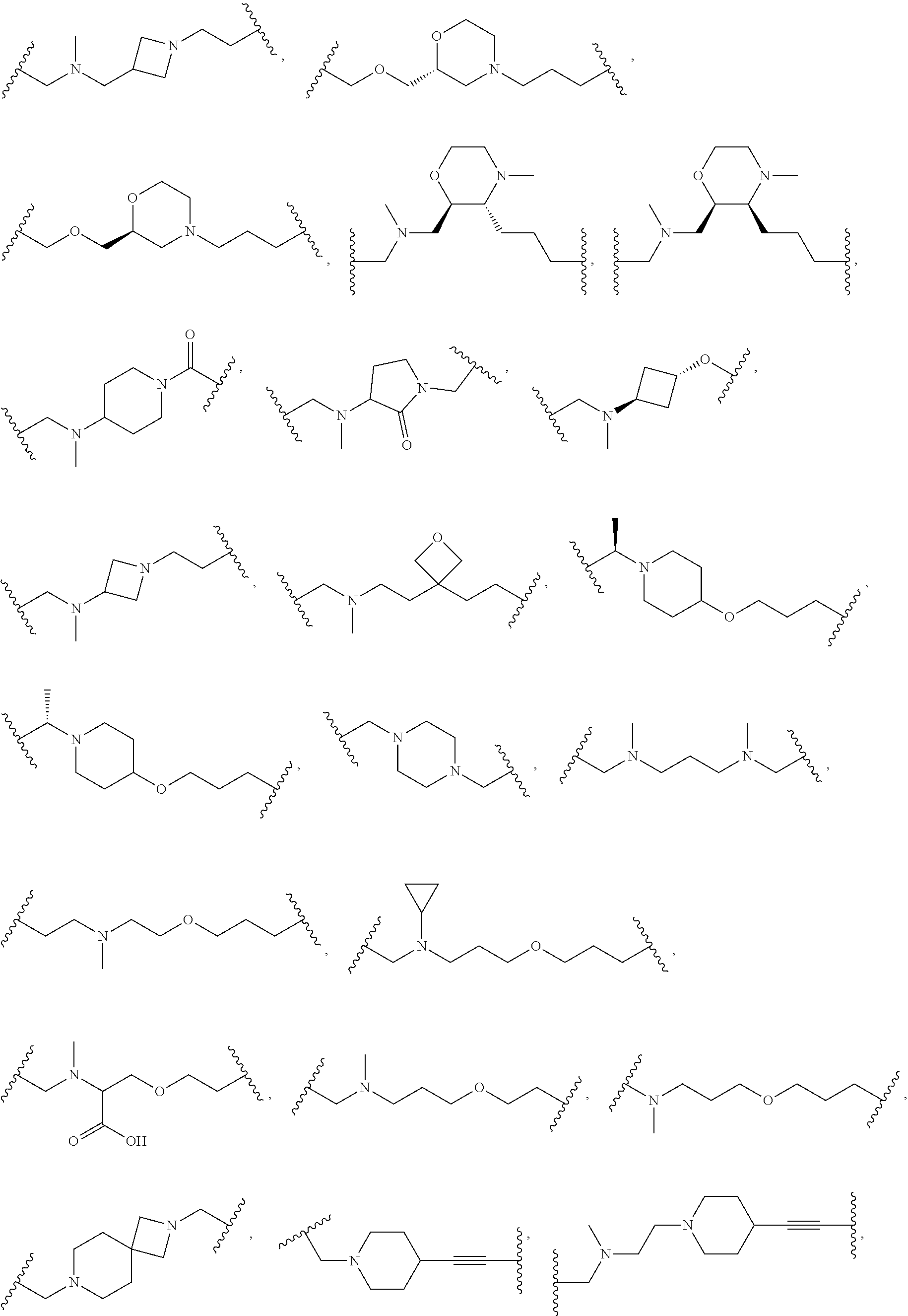

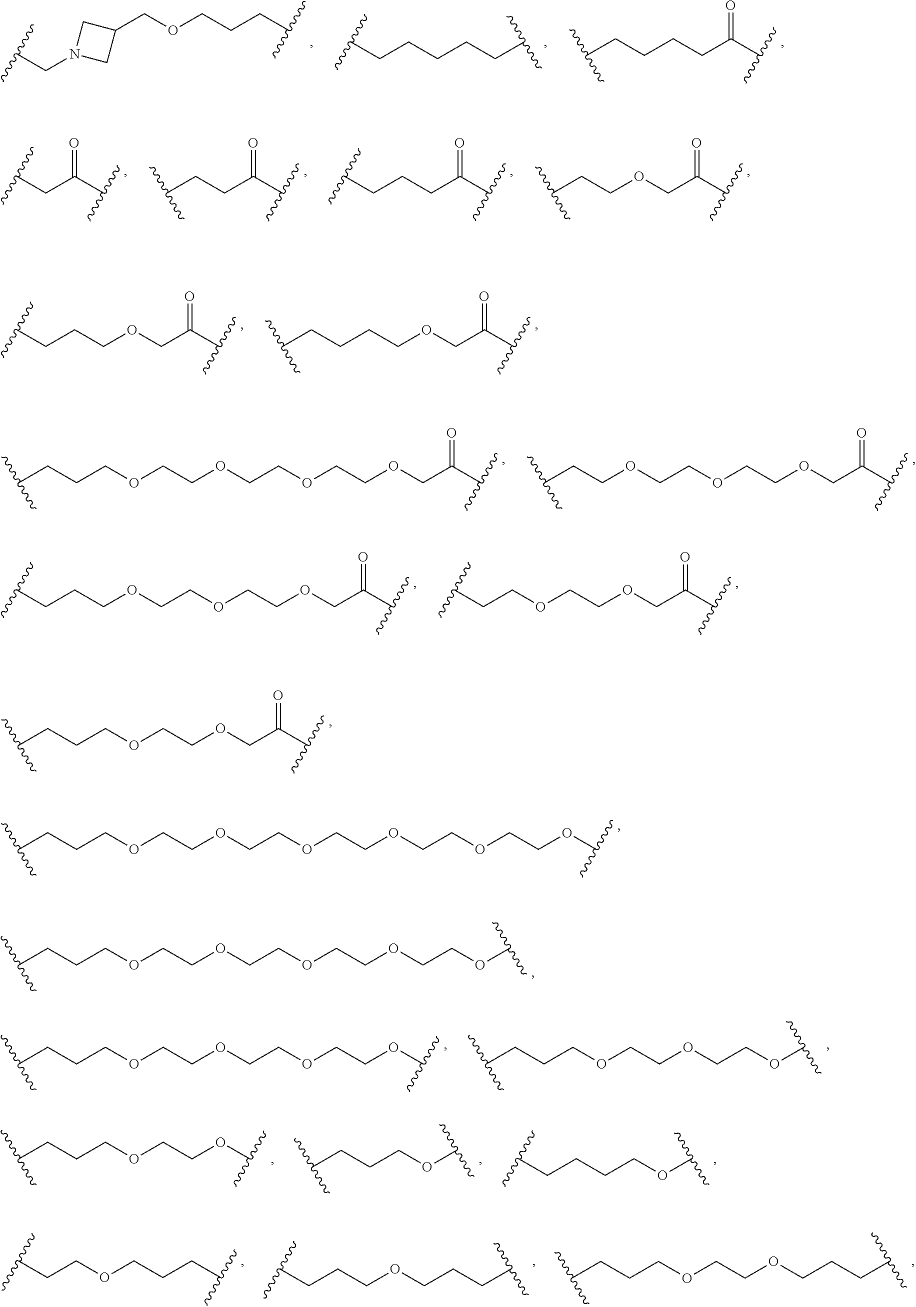

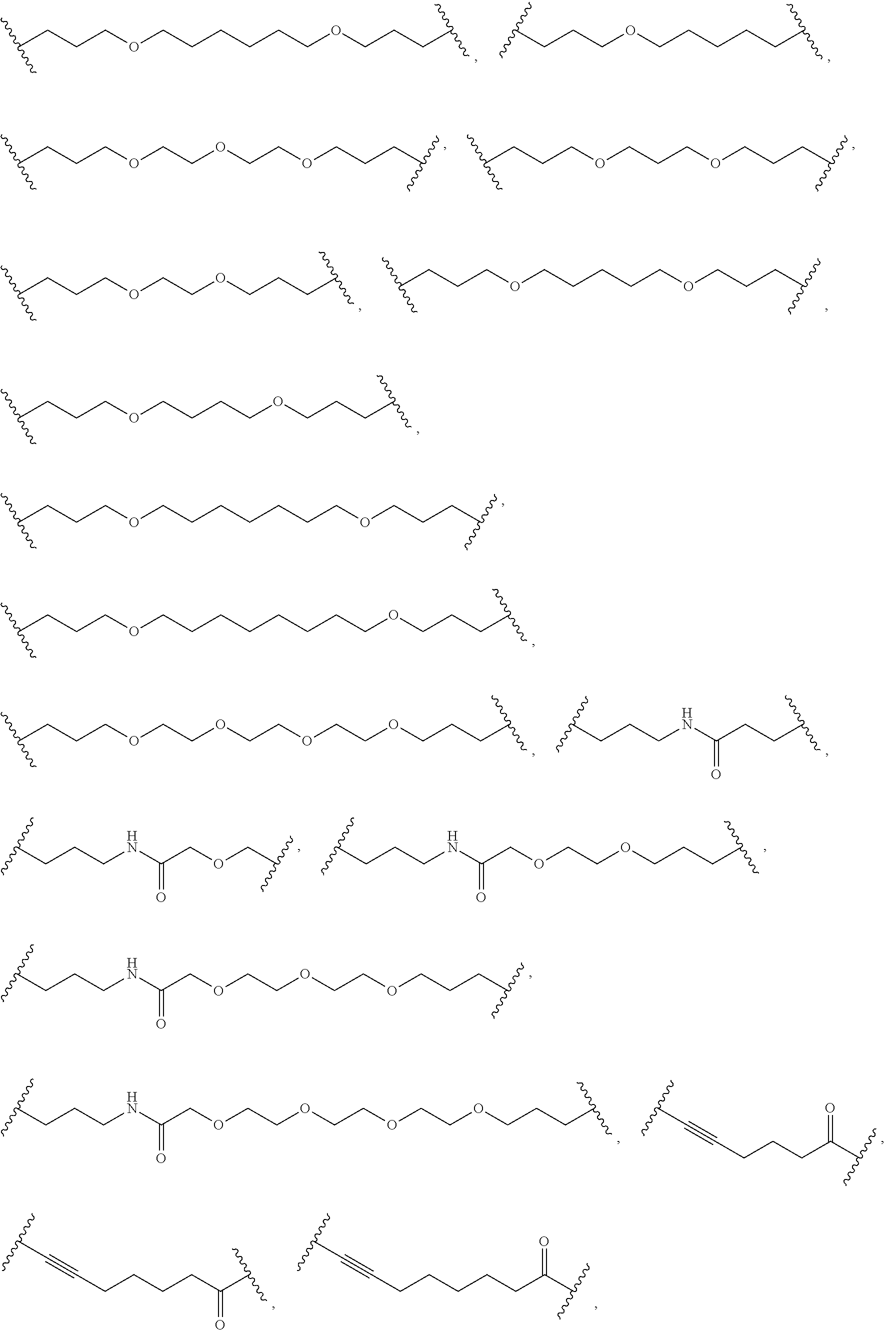

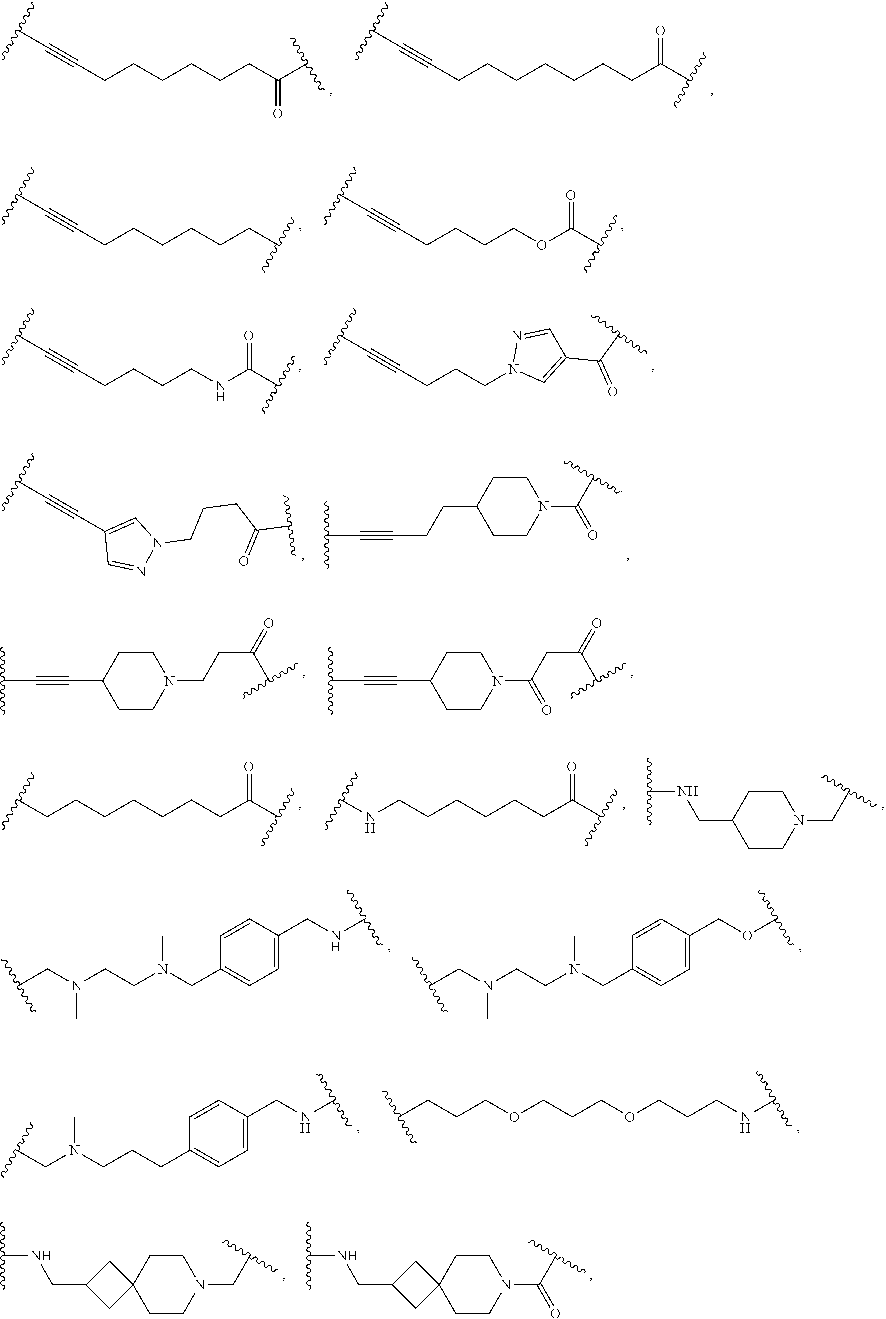

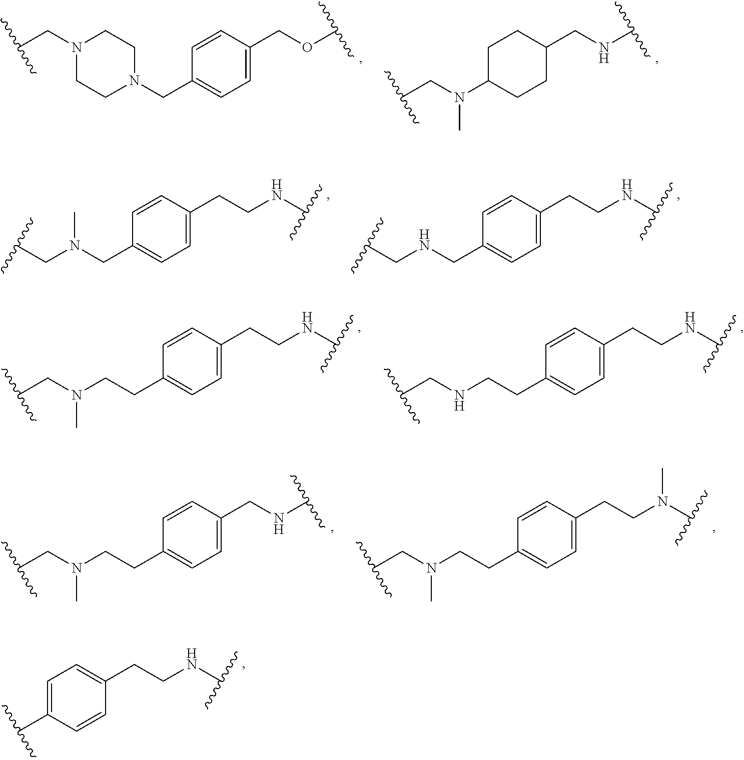

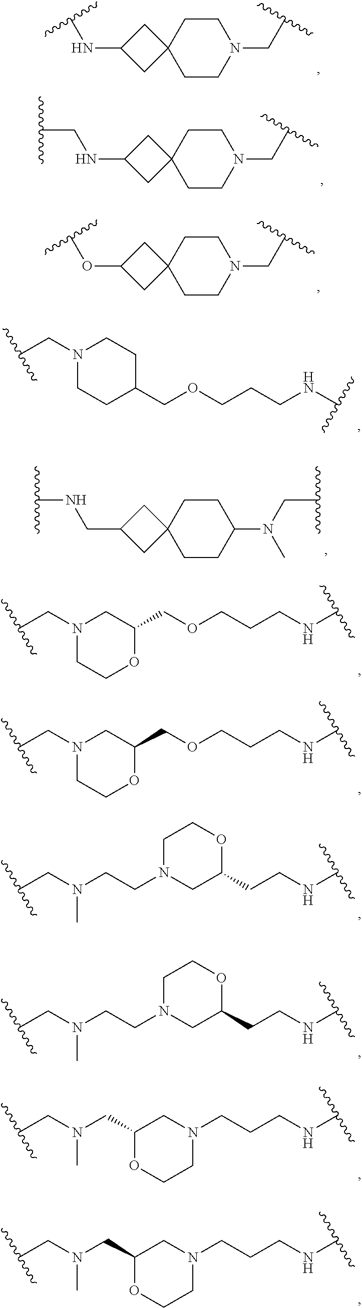

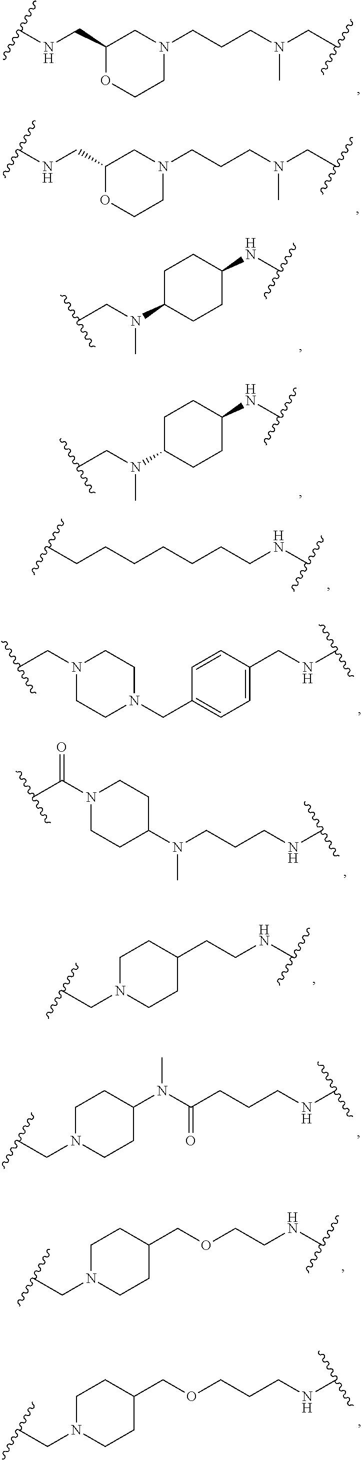

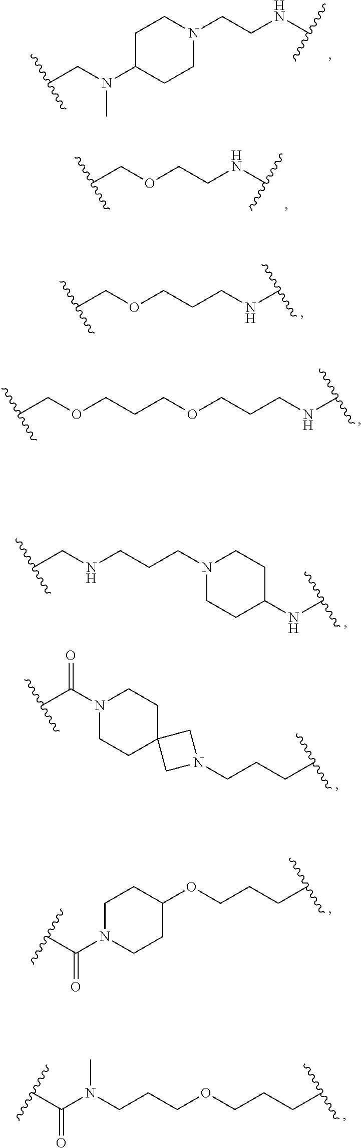

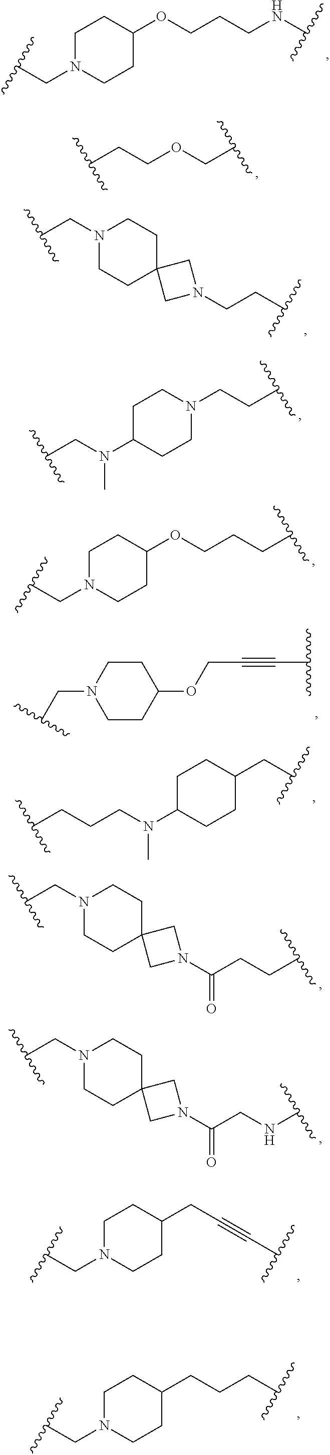

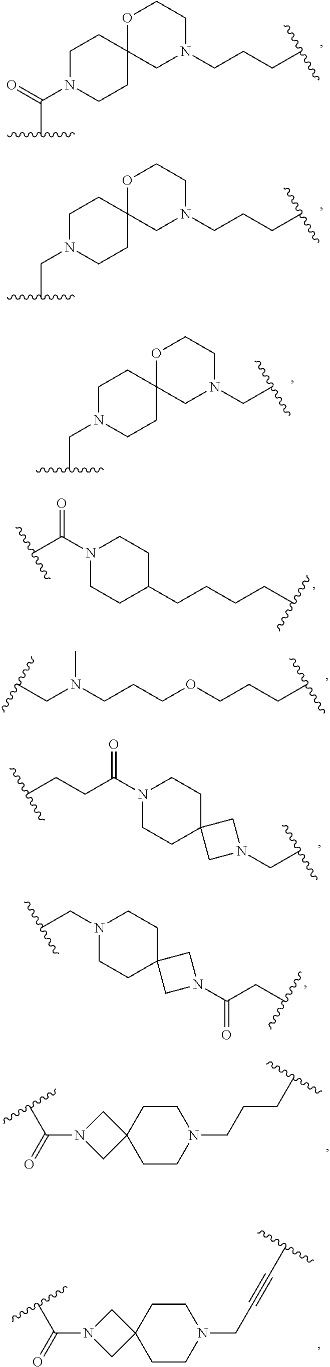

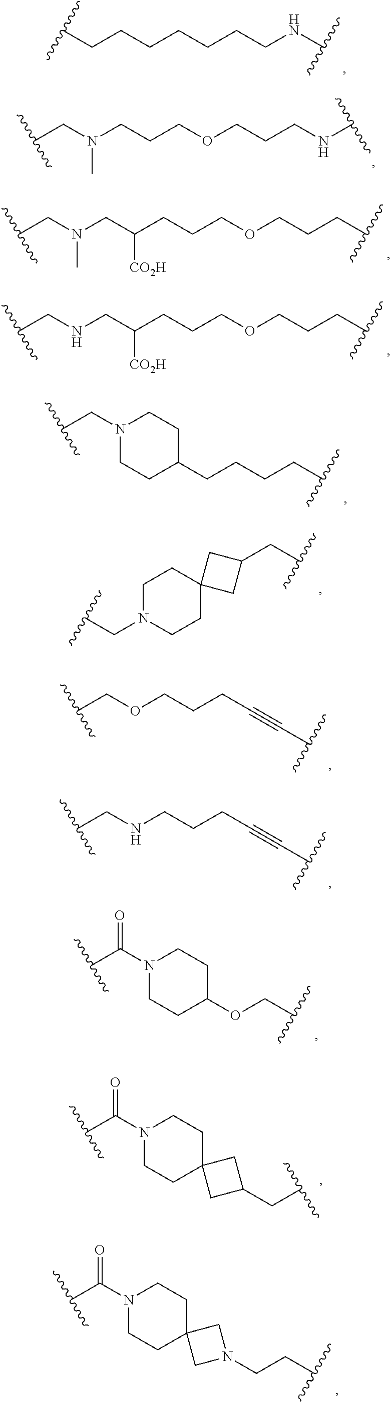

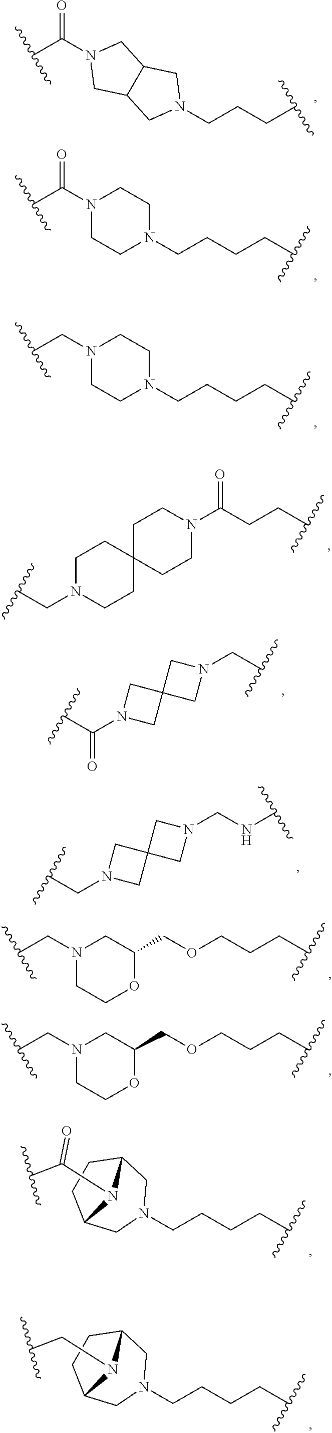

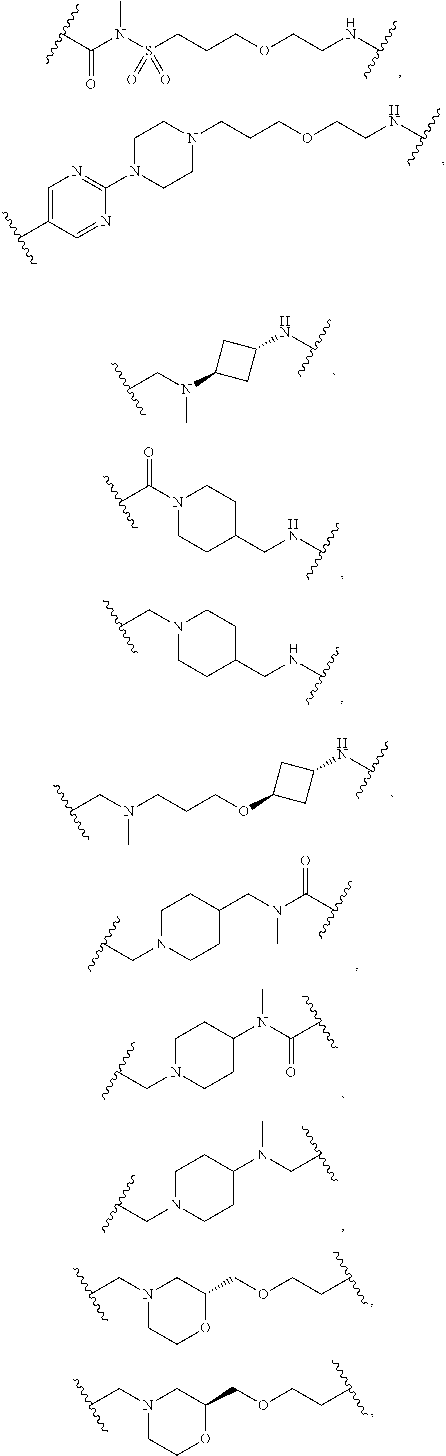

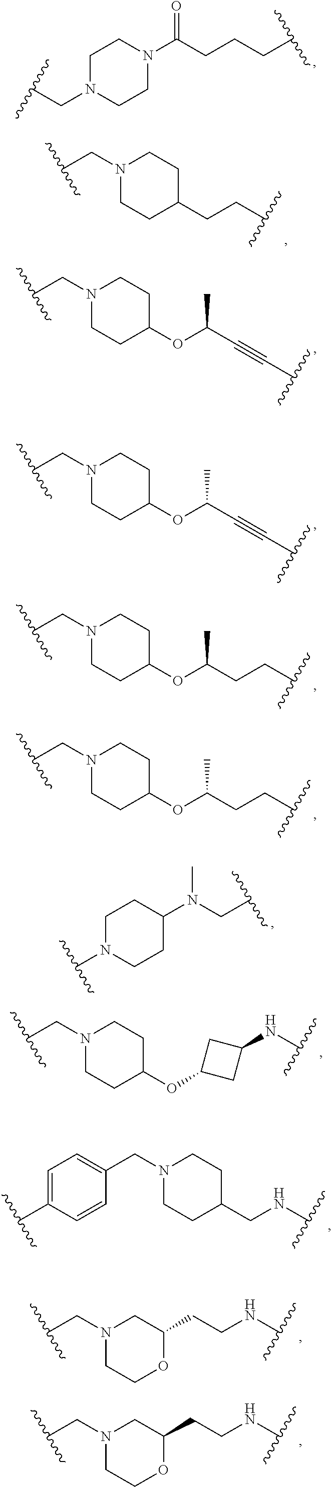

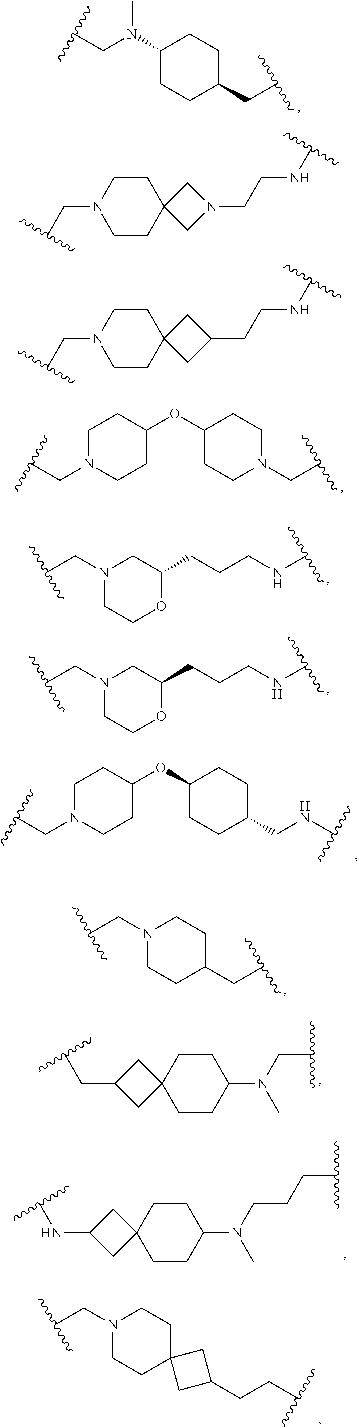

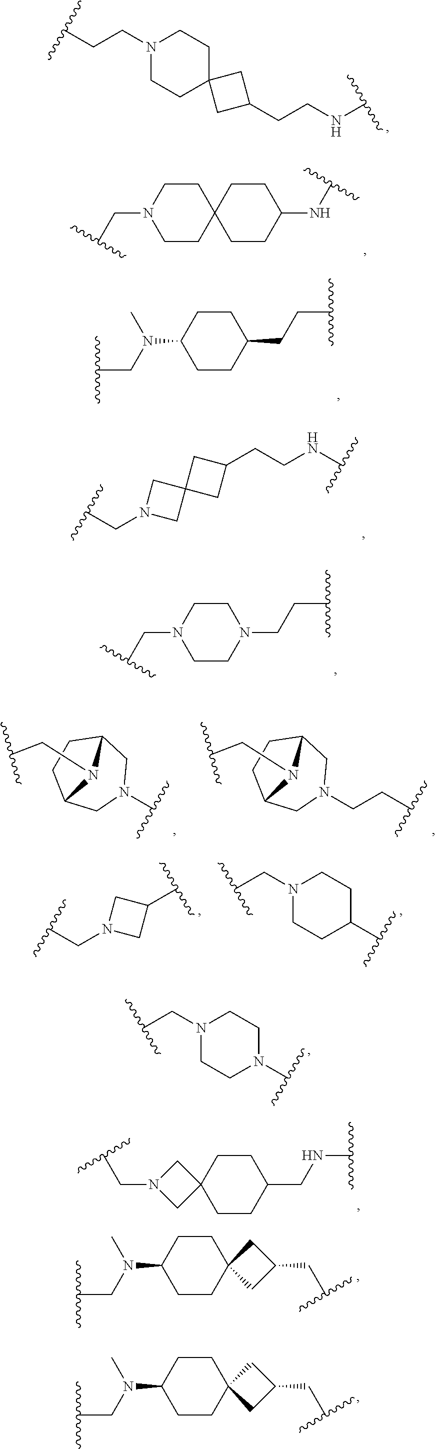

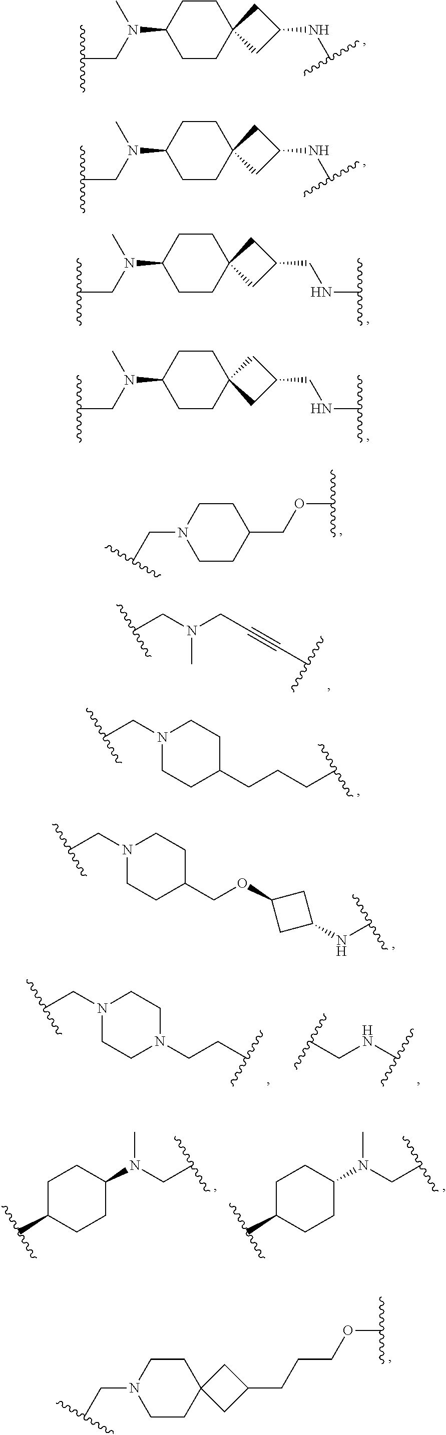

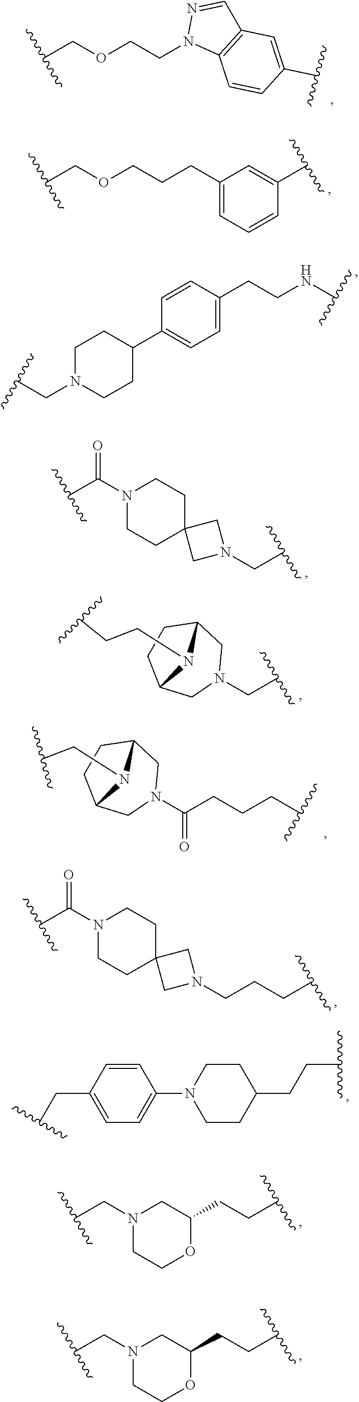

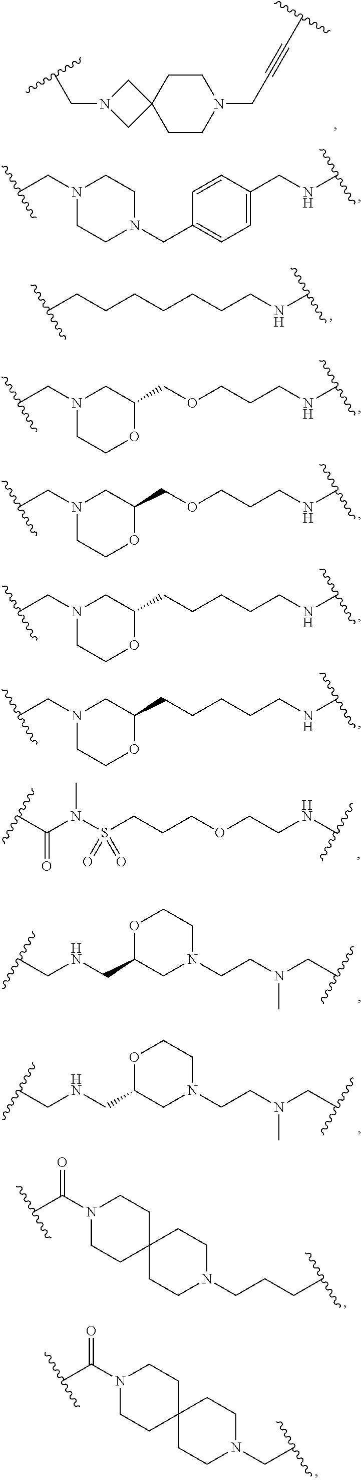

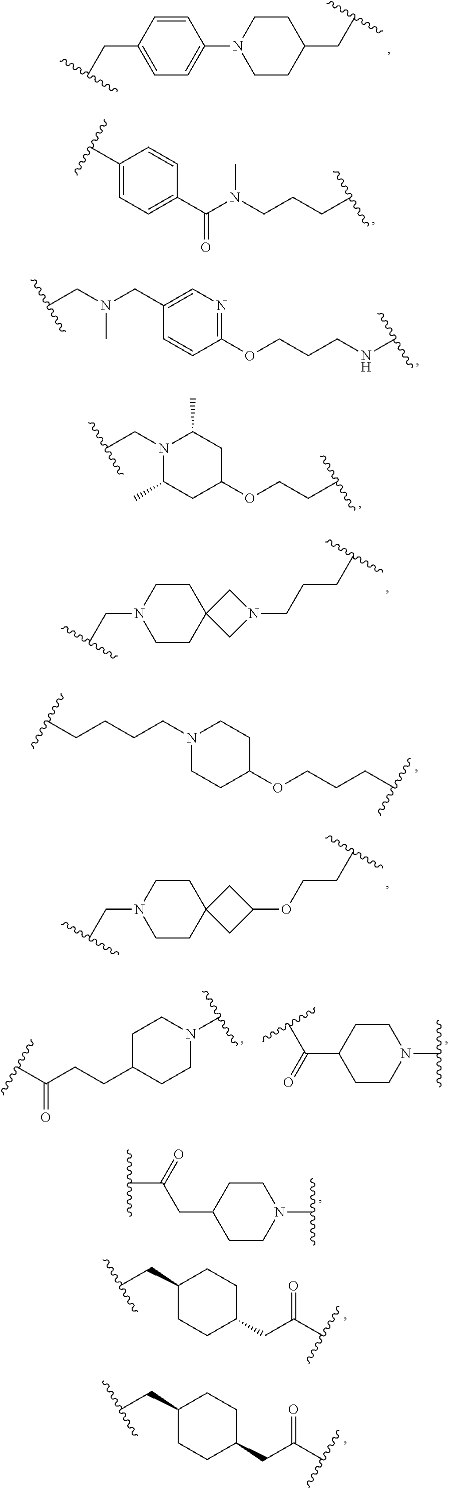

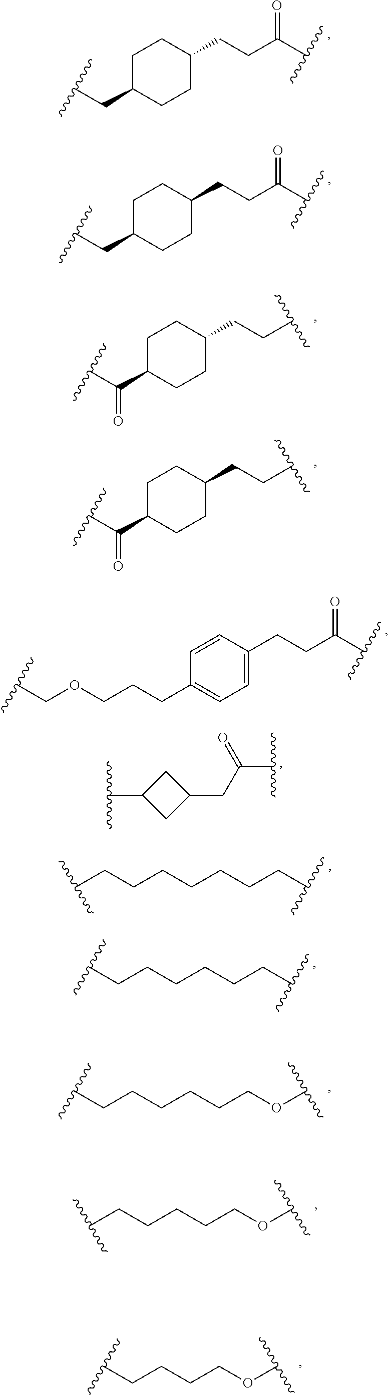

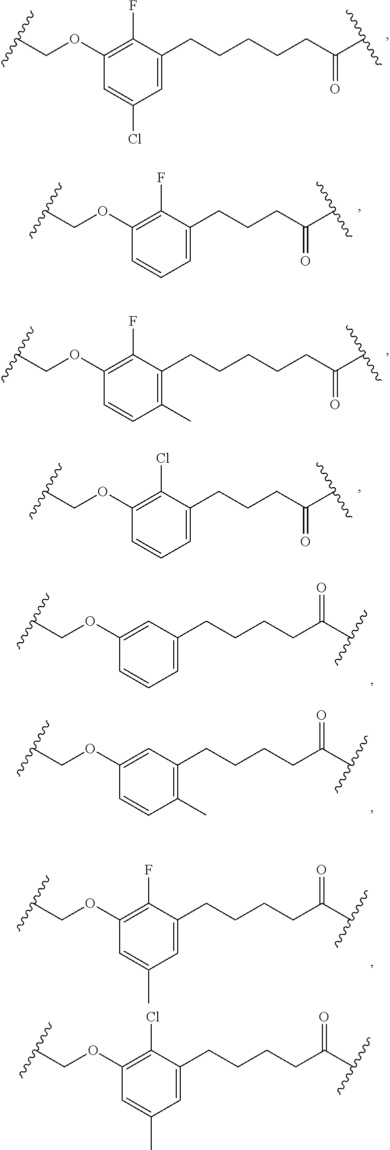

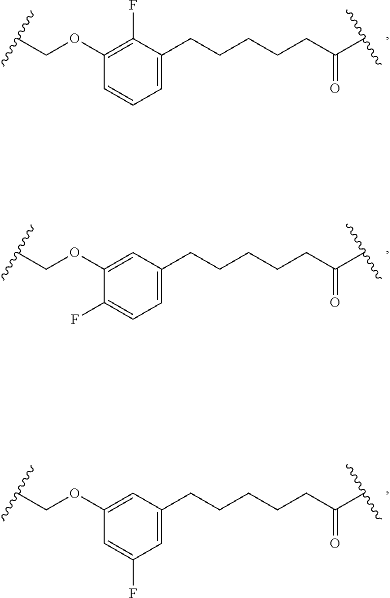 [SMILES: *COc1cccc(CCCCCC(*)=O)c1F ; *COc1cc(CCCCCC(*)=O)ccc1F ; *COc1cc(F)cc(CCCCCC(*)=O)c1]

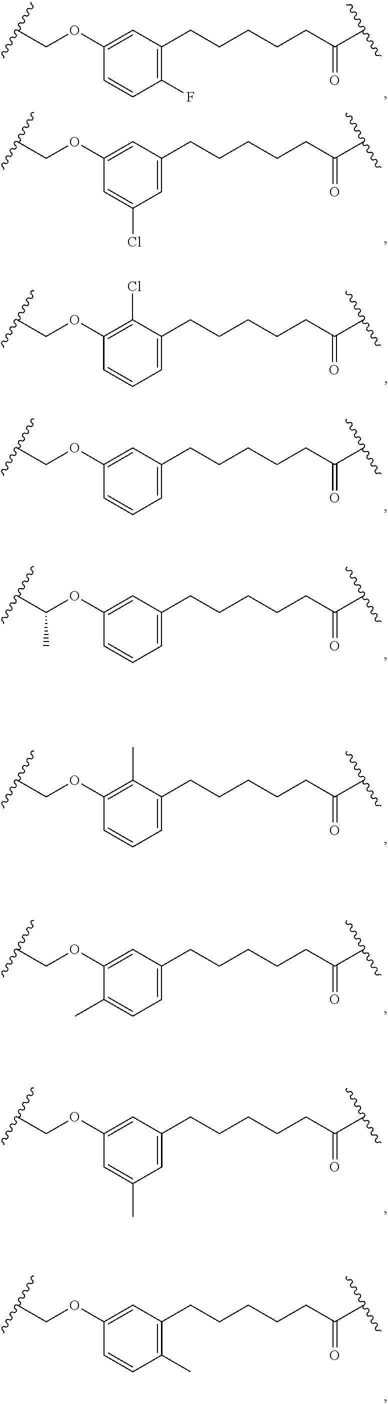

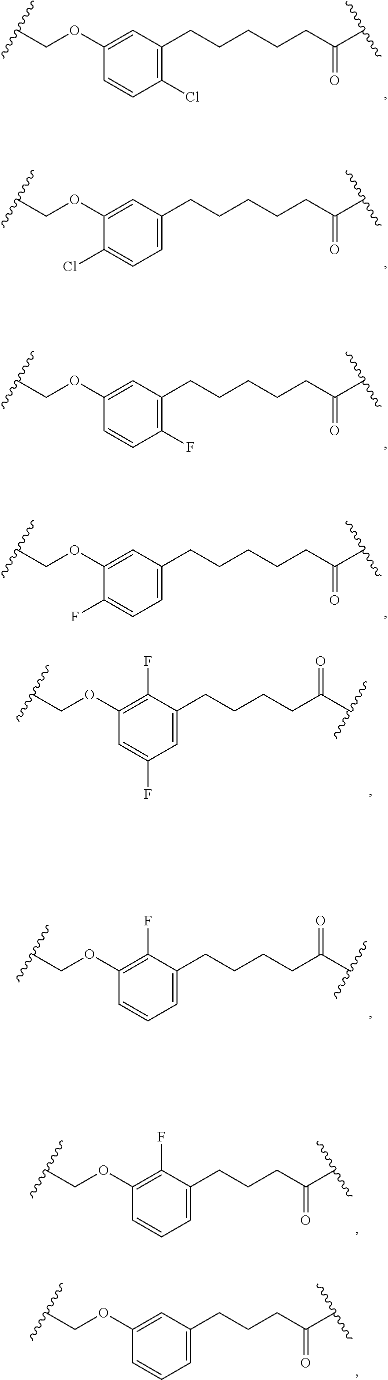

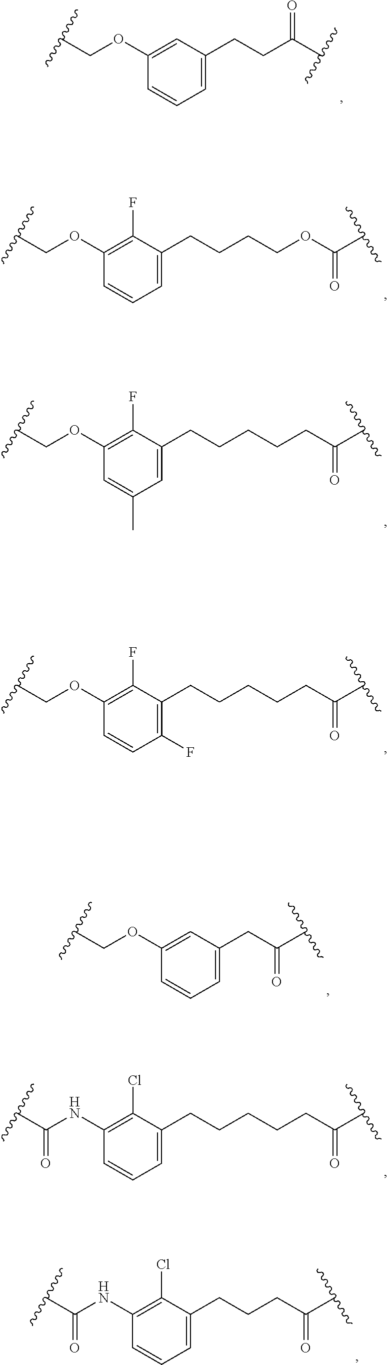

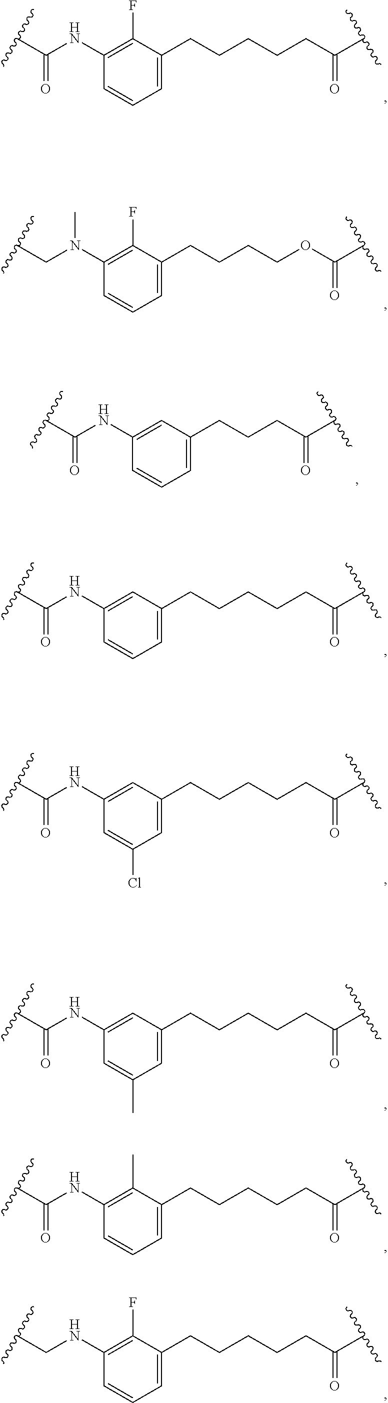

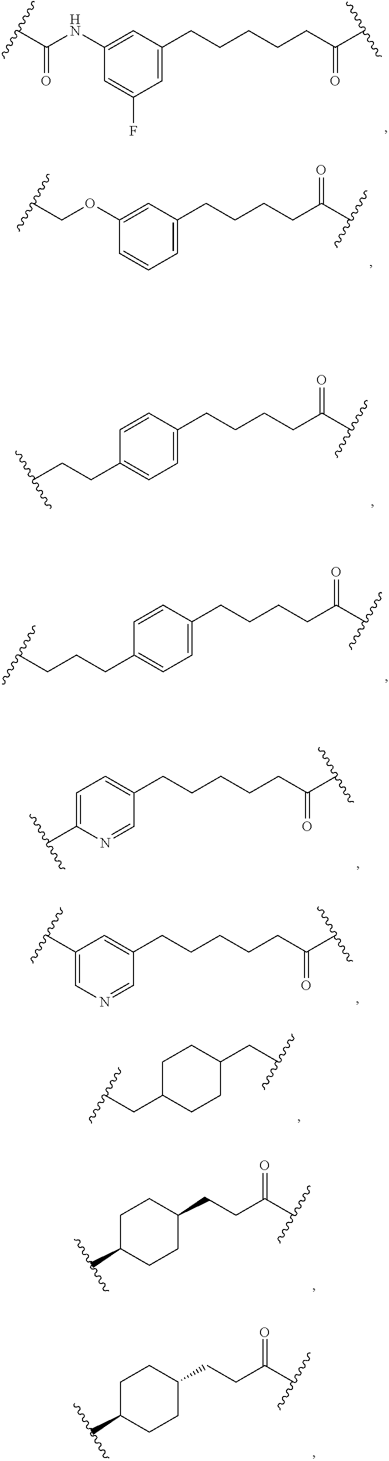

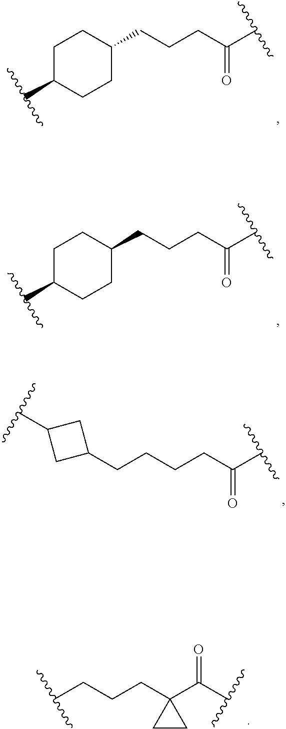 [SMILES: *C(=O)CCC[C@H]1CC[C@H](*)CC1 ; *C(=O)CCC[C@H]1CC[C@@H](*)CC1 ; *C(=O)CCCCC1CC(*)C1 ; *CCCC1(C(*)=O)CC1]

In an embodiment, L is selected from

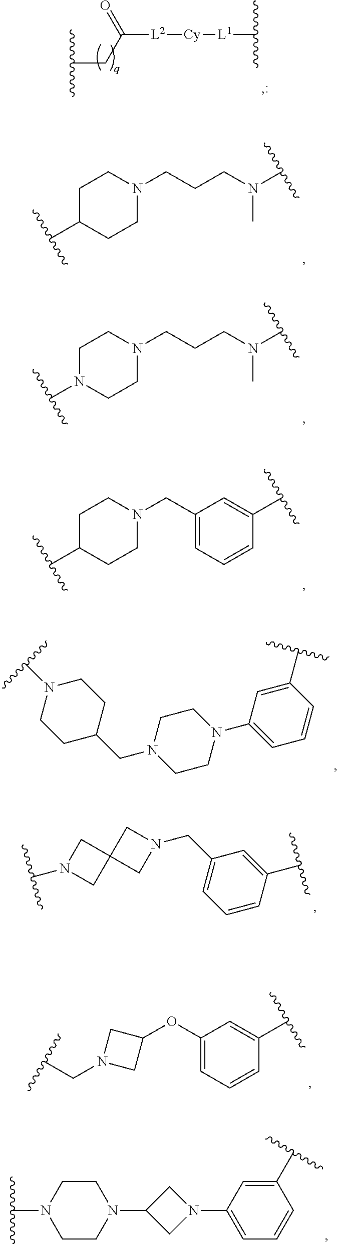

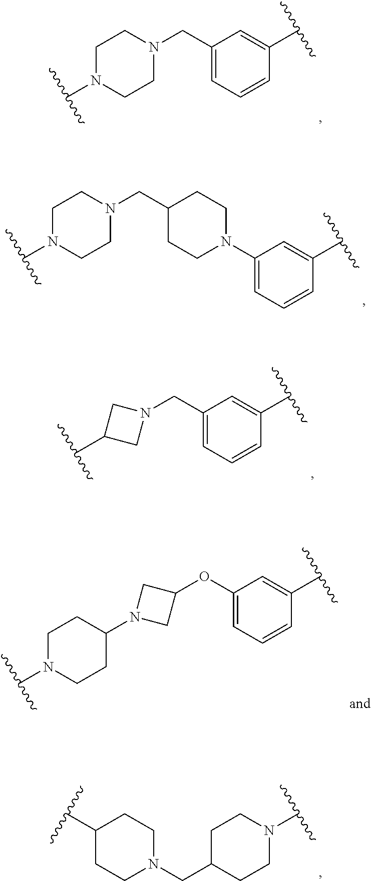

wherein L, L², —Cy— and q are as defined in any of the embodiments described herein and wherein the left attachment point connects to LBM and the right attachment point connects to the —S(O)₂— group of Formula I or the X group of Formula A-I.

In an embodiment, L is selected from

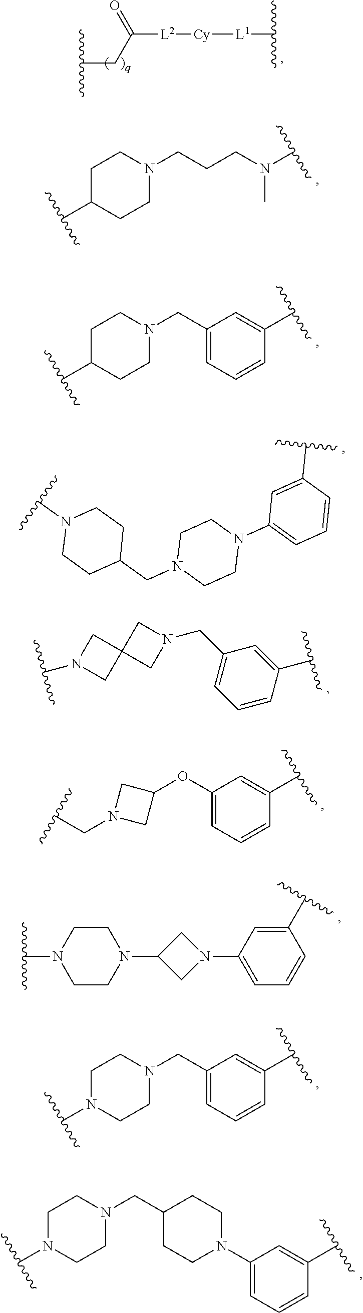

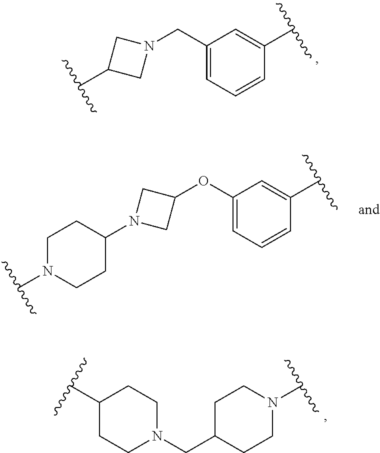

wherein L¹, L², —Cy— and q are as defined in any of the embodiments described herein and wherein the left attachment point connects to LBM and the right attachment point connects to the —S(O)₂— group of Formula I or the X group of Formula A-I.

In an embodiment, L is selected from:

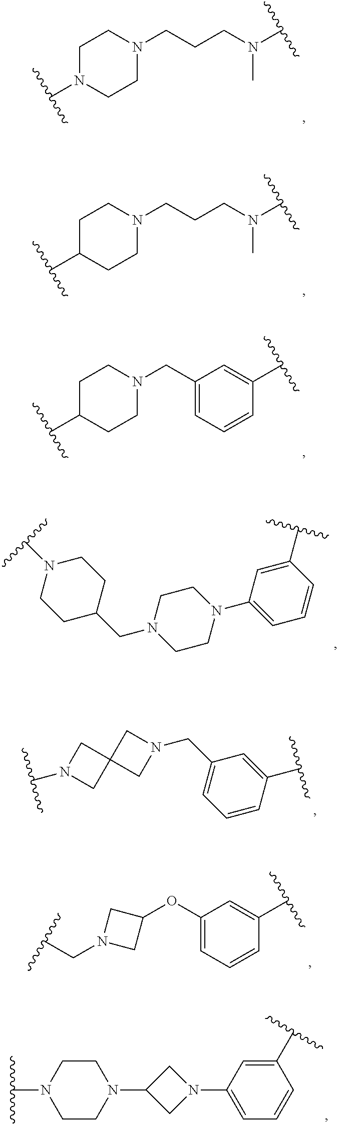

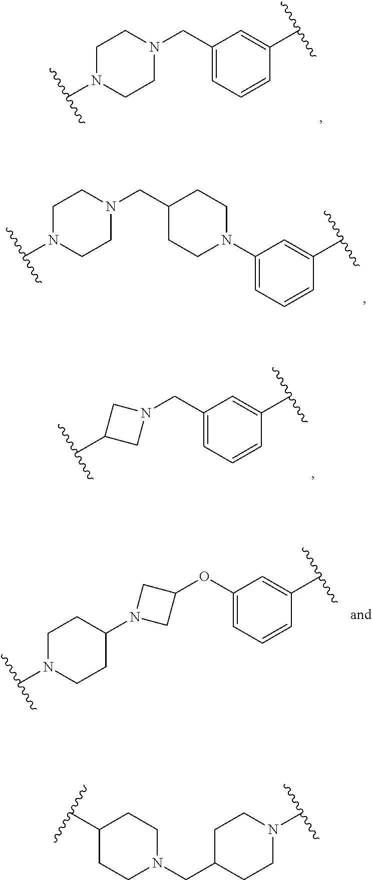 [SMILES: *c1cccc(CN2CCN(*)CC2)c1 ; *c1cccc(N2CCC(CN3CCN(*)CC3)CC2)c1 ; *c1cccc(CN2CC(*)C2)c1 ; *c1cccc(OC2CN(C3CCN(*)CC3)C2)c1 ; *C1CCN(CC2CCN(*)CC2)CC1]

wherein the left attachment point connects to LBM and the right attachment point connects to the —S(O)₂— group of Formula I or the X group of Formula A-I.

In an embodiment, L is selected from:

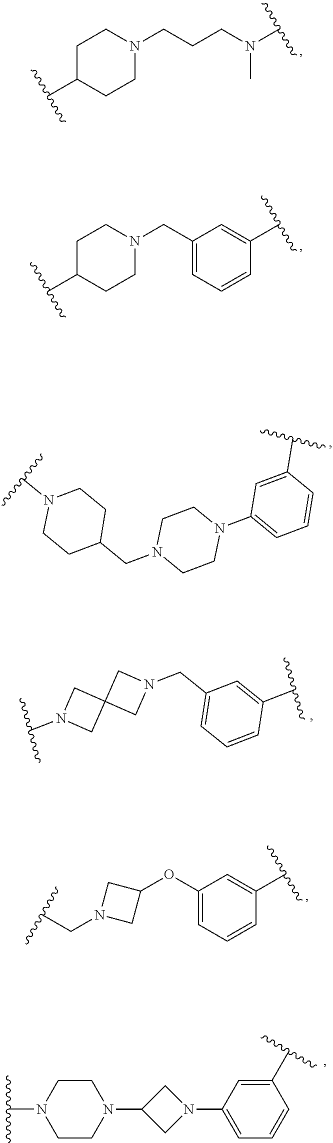 [SMILES: *C1CCN(CCCN(*)C)CC1 ; *c1cccc(CN2CCC(*)CC2)c1 ; *c1cccc(N2CCN(CC3CCN(*)CC3)CC2)c1 ; *c1cccc(CN2CC3(CN(*)C3)C2)c1 ; *CN1CC(Oc2cccc(*)c2)C1 ; *c1cccc(N2CC(N3CCN(*)CC3)C2)c1]

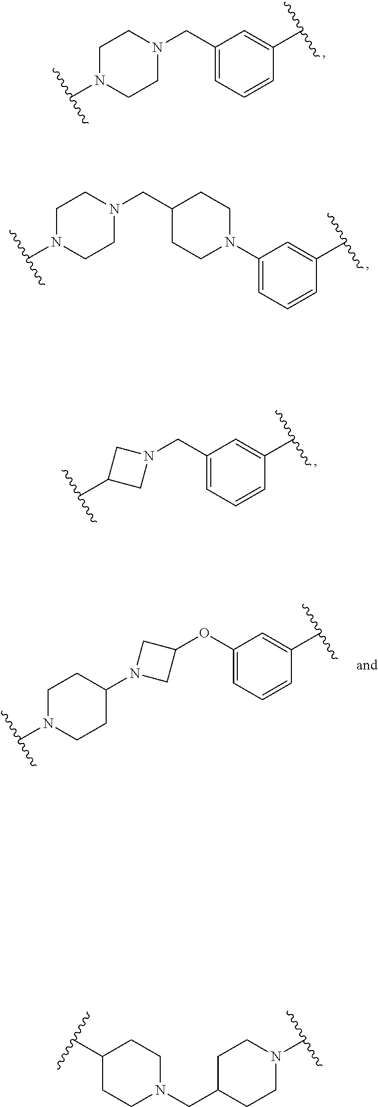 [SMILES: *c1cccc(CN2CCN(*)CC2)c1 ; *c1cccc(N2CCC(CN3CCN(*)CC3)CC2)c1 ; *c1cccc(CN2CC(*)C2)c1 ; *c1cccc(OC2CN(C3CCN(*)CC3)C2)c1 ; *C1CCN(CC2CCN(*)CC2)CC1]

wherein the left attachment point connects to LBM and the right attachment point connects to the —S(O)₂— group of Formula I or the X group of Formula A-I.

In an embodiment, L is selected from:

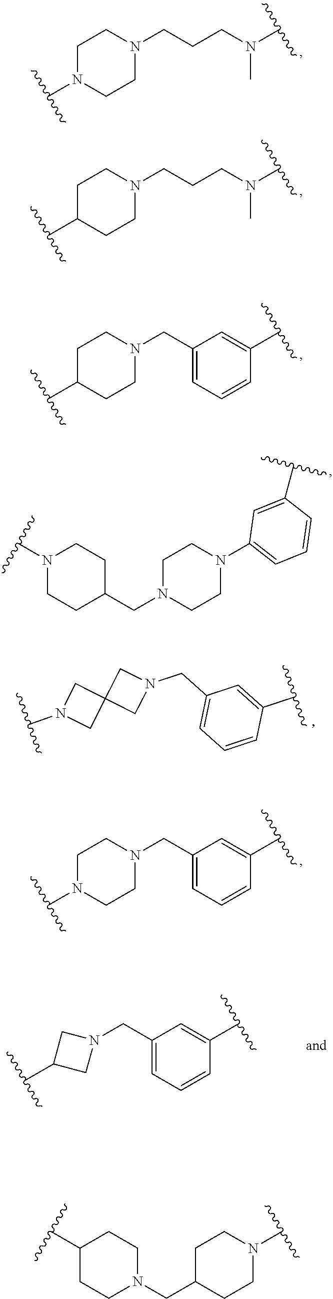

wherein the left attachment point connects to LBM and the right attachment point connects to the —S(O)₂— group of Formula I or the X group of Formula A-I.

In an embodiment, L is selected from:

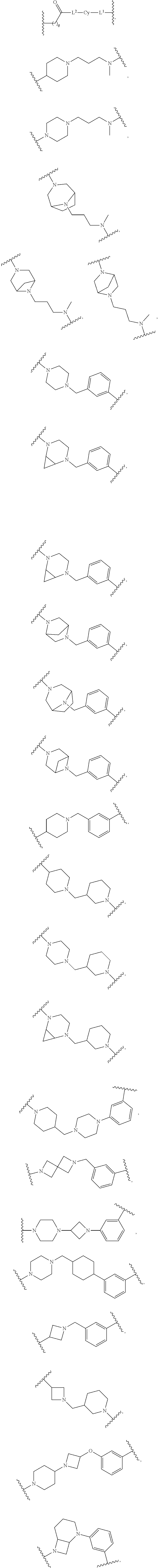

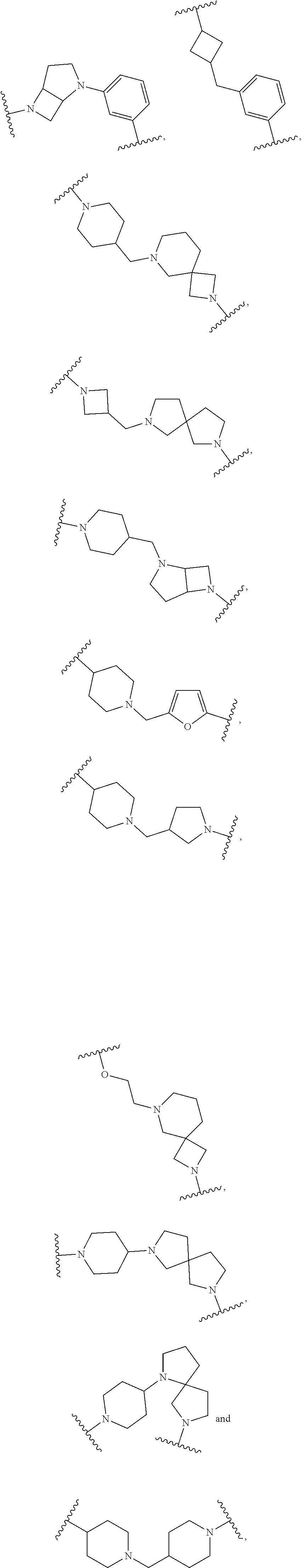

each substituted with 0, 1, 2 or 3 instances of R⁷, wherein each R⁷ is as defined in any of the embodiments described herein; wherein the left attachment point connects to LBM and the right attachment point connects to the —X— group of Formula A-I or —S(O)₂— group of Formula I and wherein
L¹ and L² are each independently selected from a bond and —N(R′), wherein R′ is selected from H and C₁₋₆ alkyl; and
q is 0 01,2,3,4,5,6,7,8,9 or 10.

In an embodiment, L is selected from

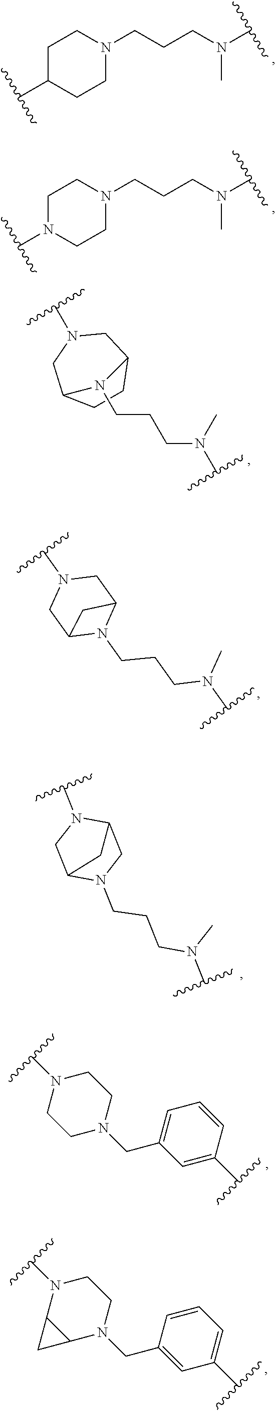

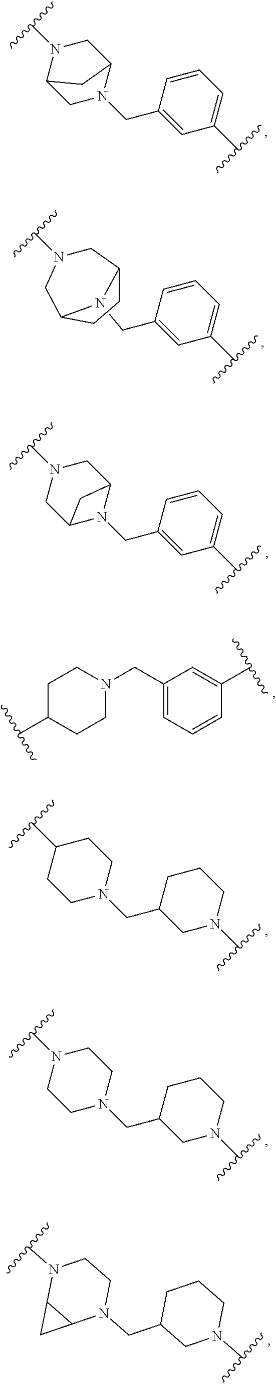

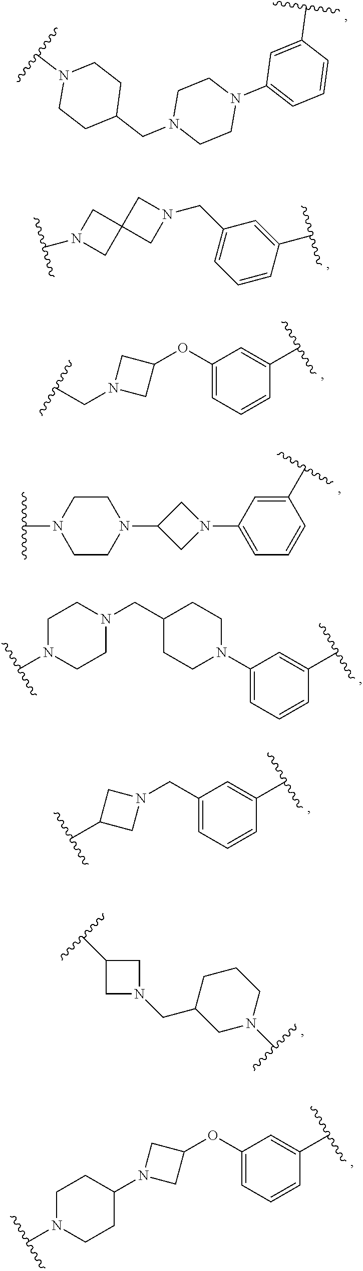

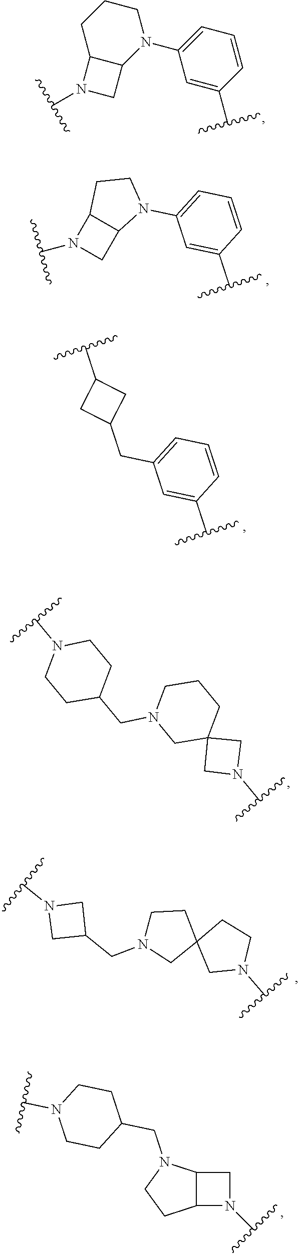 [SMILES: *c1cccc(N2CCCC3C2CN3*)c1 ; *c1cccc(N2CCC3C2CN3*)c1 ; *c1cccc(CC2CC(*)C2)c1 ; *N1CCC(CN2CCCC3(CN(*)C3)C2)CC1 ; *N1CC(CN2CCC3(CCN(*)C3)C2)C1 ; *N1CCC(CN2CCC3C2CN3*)CC1]

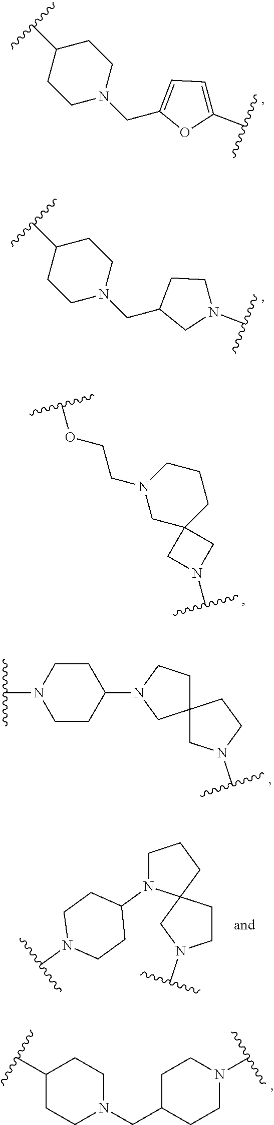 [SMILES: *c1ccc(CN2CCC(*)CC2)o1 ; *C1CCN(CC2CCN(*)C2)CC1 ; *OCCN1CCCC2(CN(*)C2)C1 ; *N1CCC(N2CCC3(CCN(*)C3)C2)CC1 ; *N1CCC(N2CCCC23CCN(*)C3)CC1 ; *C1CCN(CC2CCN(*)CC2)CC1]

each substituted with 0, 1, 2 or 3 instances of R⁷ wherein each R⁷ is as defined in any of the embodiments described herein;
wherein the left attachment point connects to LBM and the right attachment point connects to the —X— group of Formula A-I or —S(O)₂— group of Formula I.

In an embodiment, L is selected from:

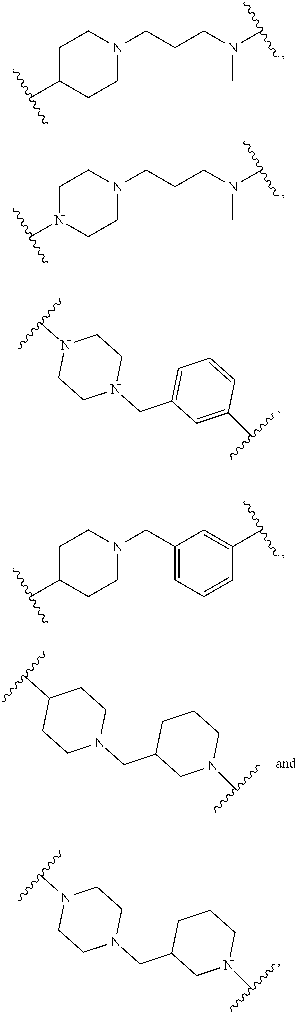 [SMILES: *C1CCN(CCCN(*)C)CC1 ; *N(C)CCCN1CCN(*)CC1 ; *c1cccc(CN2CCN(*)CC2)c1 ; *c1cccc(CN2CCC(*)CC2)c1 ; *C1CCN(CC2CCCN(*)C2)CC1 ; *N1CCN(CC2CCCN(*)C2)CC1]

each substituted with 0, 1, 2 or 3 instances of R⁷, wherein each R⁷ is as defined n any of the embodiments described herein;
wherein the left attachment point connects to LBM and the right attachment point connects to the —X— group of Formula A-I or —S(O)₂— group of Formula I.

In an embodiment, L is

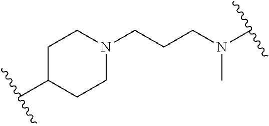 [SMILES: *C1CCN(CCCN(*)C)CC1]

substituted with 0, 1, 2 or 3 instances of R⁷, wherein each R⁷ is as defined n any of the embodiments described herein; wherein the left attachment point connects to LBM and the right attachment point connects to the —X— group of Formula A-I or —S(O)₂— group of Formula I. In an embodiment, L is

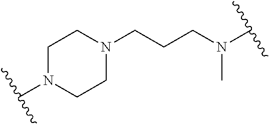 [SMILES: *N(C)CCCN1CCN(*)CC1]

substituted with 0, 1, 2 or 3 instances of R⁷, wherein each R⁷ is as defined n any of the embodiments described herein; wherein the left attachment point connects to LBM and the right attachment point connects to the —X— group of Formula A-I or —S(O)₂— group of Formula I. In an embodiment, L is

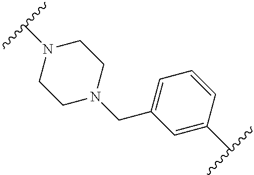 [SMILES: *c1cccc(CN2CCN(*)CC2)c1]

substituted with 0, 1, 2 or 3 instances of R⁷, wherein each R⁷ is as defined n any of the embodiments described herein; wherein the left attachment point connects to LBM and the right attachment point connects to the —X— group of Formula A-I or —S(O)₂— group of Formula I. In an embodiment, L is

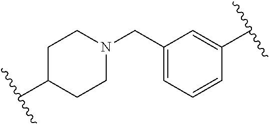 [SMILES: *c1cccc(CN2CCC(*)CC2)c1]

substituted with 0, 1, 2 or 3 instances of R⁷, wherein each R⁷ is as defined n any of the embodiments described herein; wherein the left attachment point connects to LBM and the right attachment point connects to the —X— group of Formula A-I or —S(O)₂— group of Formula I. In an embodiment, L is

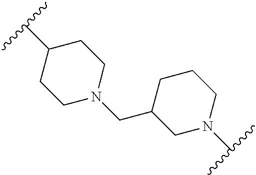 [SMILES: *C1CCN(CC2CCCN(*)C2)CC1]

substituted with 0, 1, 2 or 3 instances of R⁷, wherein each R⁷ is as defined n any of the embodiments described herein; wherein the left attachment point connects to LBM and the right attachment point connects to the —X— group of Formula A-I or —S(O)₂— group of Formula I. In an embodiment, L is

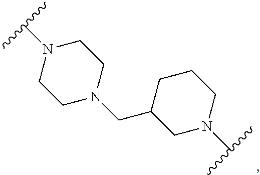 [SMILES: *N1CCN(CC2CCCN(*)C2)CC1]

substituted with 0, 1, 2 or 3 instances of R⁷, wherein each R⁷ is as defined n any of the embodiments described herein; wherein the left attachment point connects to LBM and the right attachment point connects to the —X— group of Formula A-I or —S(O)₂— group of Formula I.

In an embodiment, L is selected from:

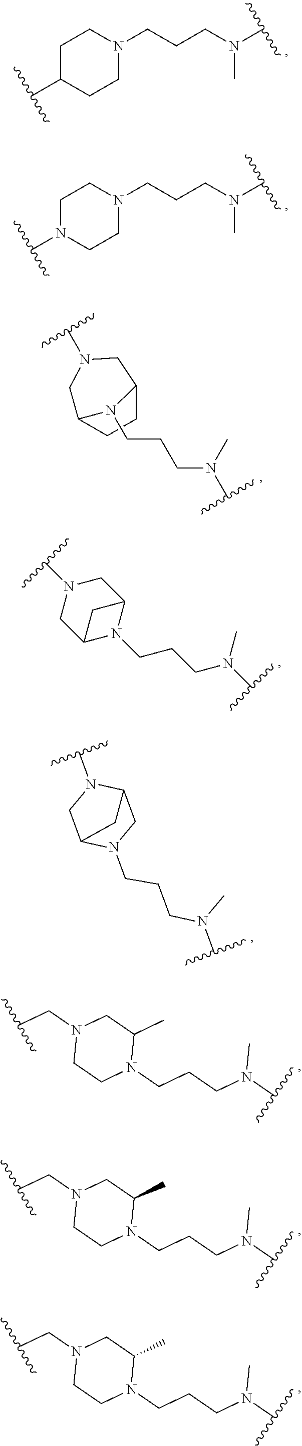

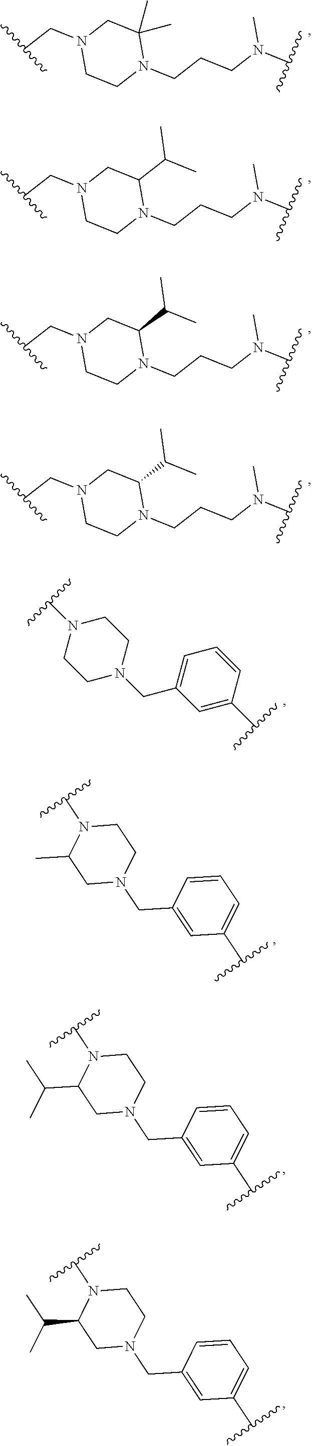

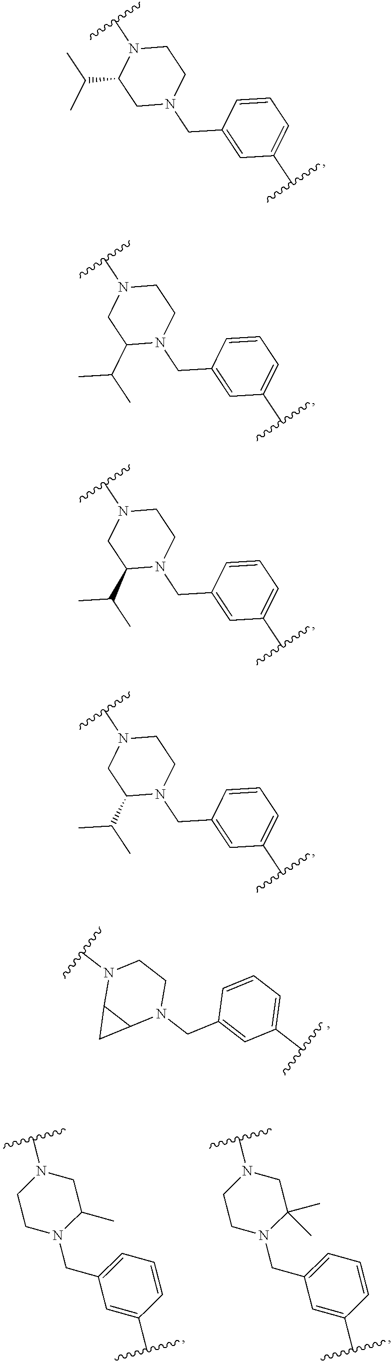

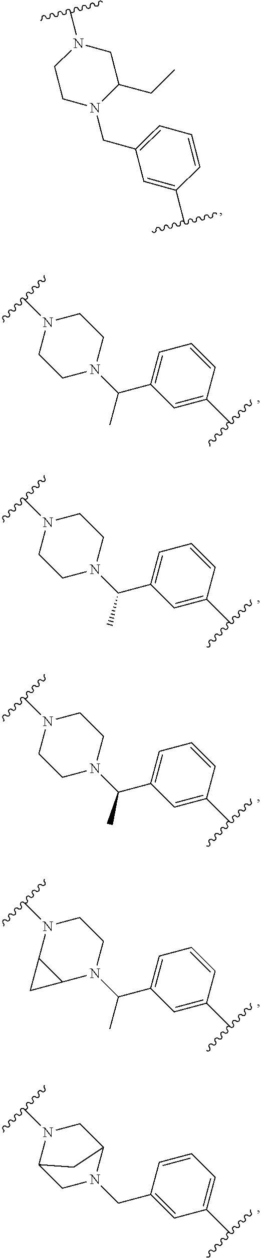 [SMILES: *c1cccc(CN2CCN(*)CC2CC)c1 ; *c1cccc(C(C)N2CCN(*)CC2)c1 ; *c1cccc([C@H](C)N2CCN(*)CC2)c1 ; *c1cccc([C@@H](C)N2CCN(*)CC2)c1 ; *c1cccc(C(C)N2CCN(*)C3CC32)c1 ; *c1cccc(CN2CC3CC2CN3*)c1]

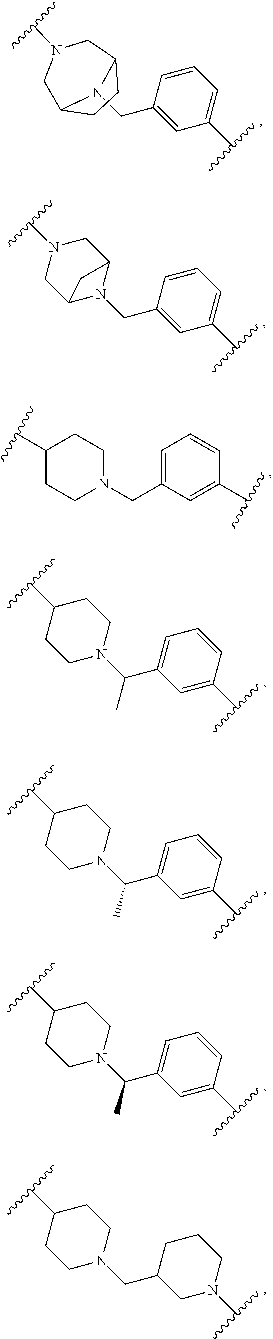

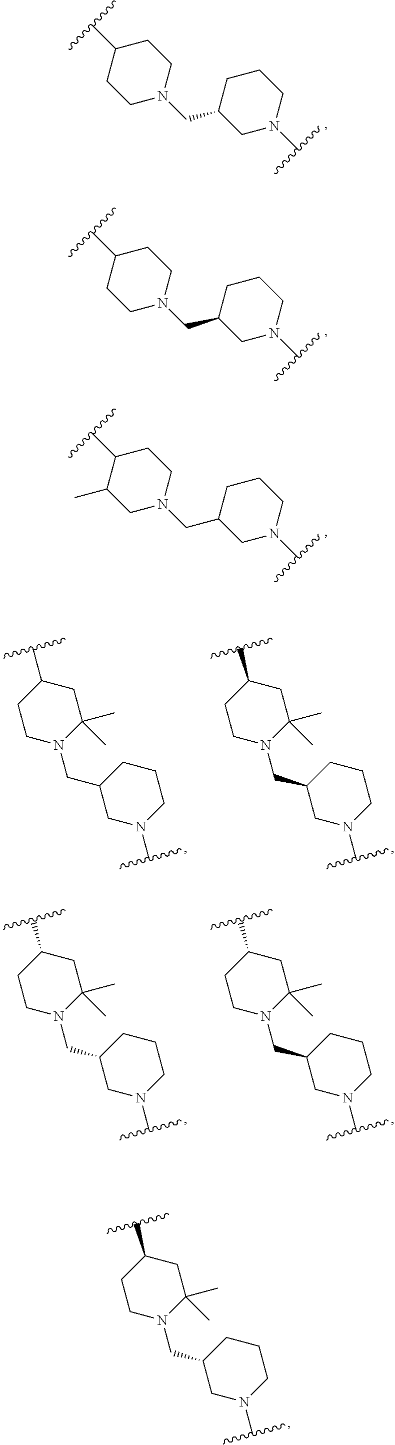

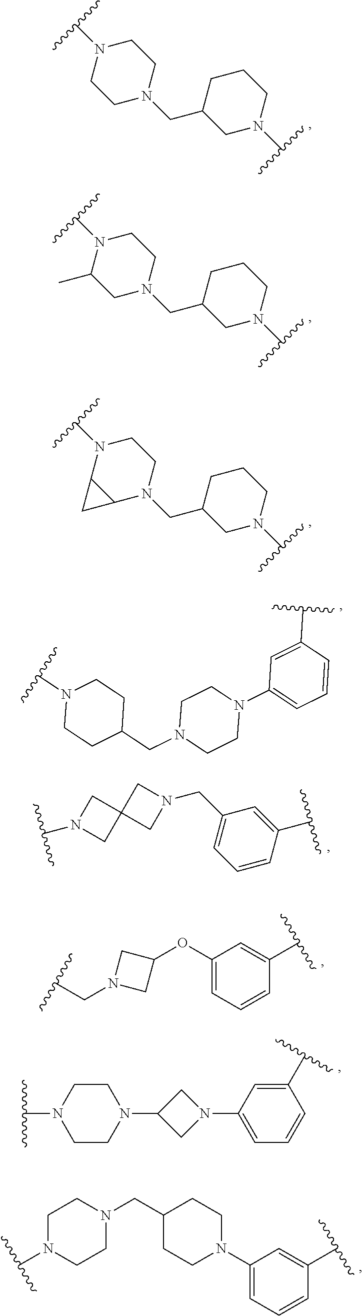

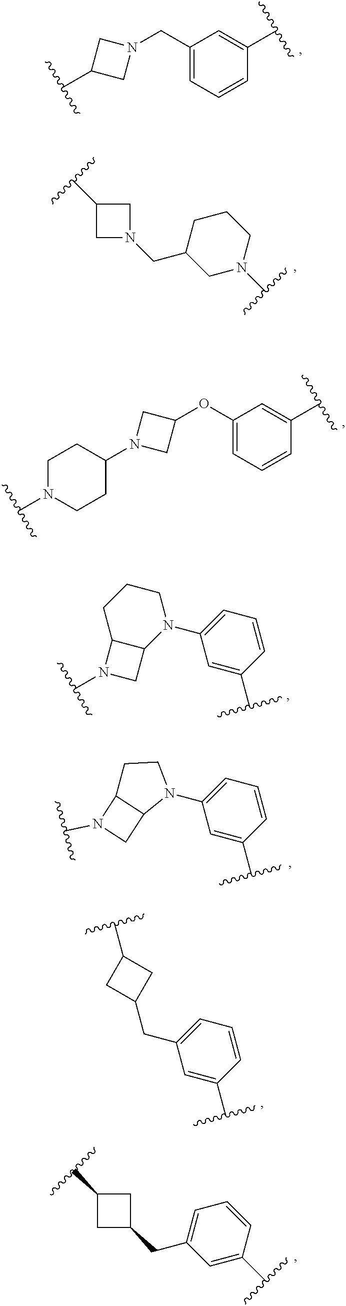

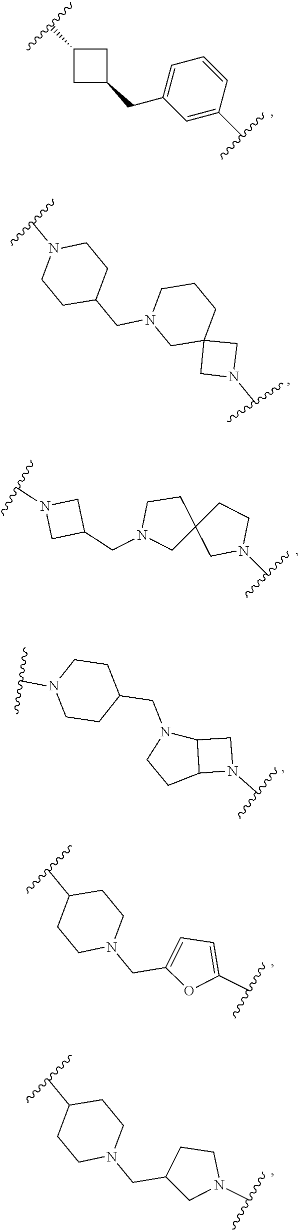 [SMILES: *c1cccc(C[C@H]2C[C@H](*)C2)c1 ; *N1CCC(CN2CCCC3(CN(*)C3)C2)CC1 ; *N1CC(CN2CCC3(CCN(*)C3)C2)C1 ; *N1CCC(CN2CCC3C2CN3*)CC1 ; *c1ccc(CN2CCC(*)CC2)o1 ; *C1CCN(CC2CCN(*)C2)CC1]

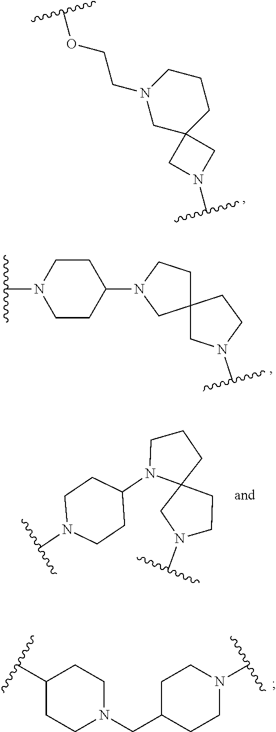 [SMILES: *OCCN1CCCC2(CN(*)C2)C1 ; *N1CCC(N2CCC3(CCN(*)C3)C2)CC1 ; *N1CCC(N2CCCC23CCN(*)C3)CC1 ; *C1CCN(CC2CCN(*)CC2)CC1]

wherein the left attachment point connects to LBM and the right attachment point connects to the —X— group of Formula A-I or —S(O)₂— group of Formula I.

In an embodiment, L is

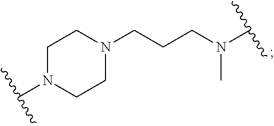 [SMILES: *N(C)CCCN1CCN(*)CC1]

wherein the left attachment point connects to LBM and the right attachment point connects to the —X— group of Formula A-I or —S(O)₂— group of Formula I. In an embodiment, L is

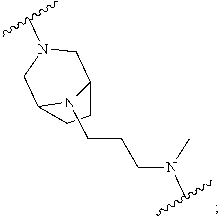 [SMILES: *N(C)CCCN1C2CCC1CN(*)C2]

wherein the left attachment point connects to LBM and the right attachment point connects to the —X— group of Formula A-I or —S(O)₂— group of Formula I. In an embodiment, L is

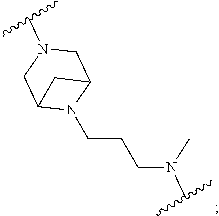 [SMILES: *N(C)CCCN1C2CC1CN(*)C2]

wherein the left attachment point connects to LBM and the right attachment point connects to the —X— group of Formula A-I or —S(O)₂— group of Formula I. In an embodiment, L is

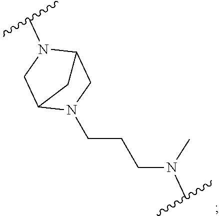 [SMILES: *N(C)CCCN1CC2CC1CN2*]

wherein the left attachment point connects to LBM and the right attachment point connects to the —X— group of Formula A-I or —S(O)₂— group of Formula I. In an embodiment, L is

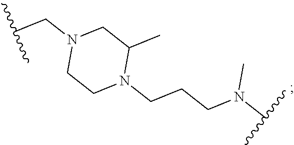 [SMILES: *CN1CCN(CCCN(*)C)C(C)C1]

wherein the left attachment point connects to LBM and the right attachment point connects to the —X— group of Formula A-I or —S(O)₂— group of Formula I. In an embodiment, L is

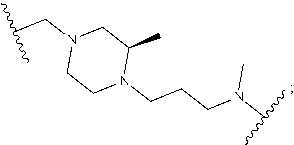 [SMILES: *CN1CCN(CCCN(*)C)[C@H](C)C1]

wherein the left attachment point connects to LBM and the right attachment point connects to the —X— group of Formula A-I or —S(O)₂— group of Formula I. In an embodiment, L is

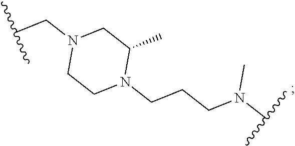 [SMILES: *CN1CCN(CCCN(*)C)[C@@H](C)C1]

wherein the left attachment point connects to LBM and the right attachment point connects to the —X— group of Formula A-I or —S(O)₂— group of Formula I. In an embodiment, L is

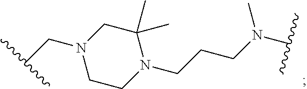 [SMILES: *CN1CCN(CCCN(*)C)C(C)(C)C1]

wherein the left attachment point connects to LBM and the right attachment point connects to the —X— group of Formula A-I or —S(O)₂— group of Formula I. In an embodiment, L is

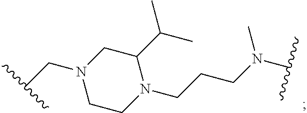 [SMILES: *CN1CCN(CCCN(*)C)C(C(C)C)C1]

wherein the left attachment point connects to LBM and the right attachment point connects to the —X— group of Formula A-I or —S(O)₂— group of Formula I. In an embodiment, L is

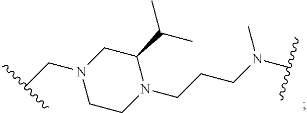 [SMILES: *CN1CCN(CCCN(*)C)[C@H](C(C)C)C1]

wherein the left attachment point connects to LBM and the right attachment point connects to the —X— group of Formula A-I or —S(O)₂— group of Formula I. In an embodiment, L is

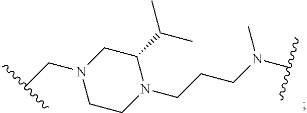 [SMILES: *CN1CCN(CCCN(*)C)[C@@H](C(C)C)C1]

wherein the left attachment point connects to LBM and the right attachment point connects to the —X— group of Formula A-I or —S(O)₂— group of Formula I. In an embodiment, L is

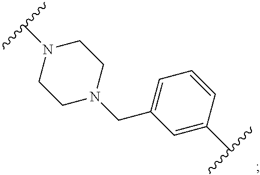 [SMILES: *c1cccc(CN2CCN(*)CC2)c1]

wherein the left attachment point connects to LBM and the right attachment point connects to the —X— group of Formula A-I or —S(O)₂— group of Formula I. In an embodiment, L is

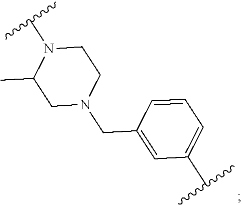 [SMILES: *c1cccc(CN2CCN(*)C(C)C2)c1]

wherein the left attachment point connects to LBM and the right attachment point connects to the —X— group of Formula A-I or —S(O)₂— group of Formula I. In an embodiment, L is

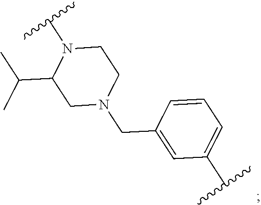 [SMILES: *c1cccc(CN2CCN(*)C(C(C)C)C2)c1]

wherein the left attachment point connects to LBM and the right attachment point connects to the —X— group of Formula A-I or —S(O)₂— group of Formula I. In an embodiment, L is

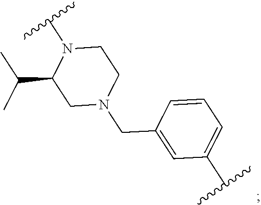 [SMILES: *c1cccc(CN2CCN(*)[C@H](C(C)C)C2)c1]

wherein the left attachment point connects to LBM and the right attachment point connects to the —X— group of Formula A-I or —S(O)₂— group of Formula I. In an embodiment, L is

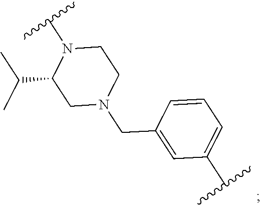 [SMILES: *c1cccc(CN2CCN(*)[C@@H](C(C)C)C2)c1]

wherein the left attachment point connects to LBM and the right attachment point connects to the —X— group of Formula A-I or —S(O)₂— group of Formula I. In an embodiment, L is

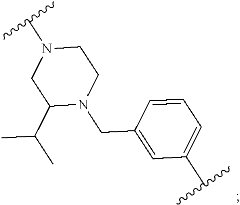 [SMILES: *c1cccc(CN2CCN(*)CC2C(C)C)c1]

wherein the left attachment point connects to LBM and the right attachment point connects to the —X— group of Formula A-I or —S(O)₂— group of Formula I. In an embodiment, L is

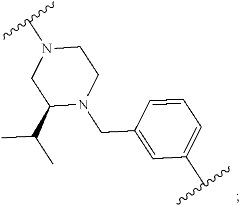 [SMILES: *c1cccc(CN2CCN(*)C[C@@H]2C(C)C)c1]

wherein the left attachment point connects to LBM and the right attachment point connects to the —X— group of Formula A-I or —S(O)₂— group of Formula I. In an embodiment, L is

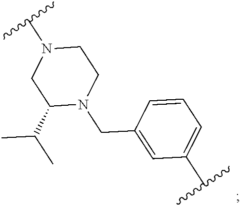 [SMILES: *c1cccc(CN2CCN(*)C[C@H]2C(C)C)c1]

wherein the left attachment point connects to LBM and the right attachment point connects to the —X— group of Formula A-I or —S(O)₂— group of Formula I. In an embodiment, L is

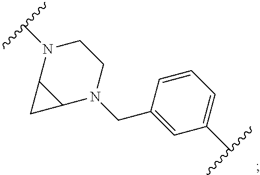 [SMILES: *c1cccc(CN2CCN(*)C3CC32)c1]

wherein the left attachment point connects to LBM and the right attachment point connects to the —X— group
of Formula A-I or —S(O)₂— group of Formula I. In an embodiment, L is

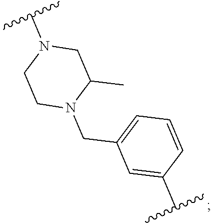 [SMILES: *c1cccc(CN2CCN(*)CC2C)c1]

wherein the left attachment point connects to LBM and the right attachment point connects to the —X— group of Formula A-I or —S(O)₂— group of Formula I. In an embodiment, L is

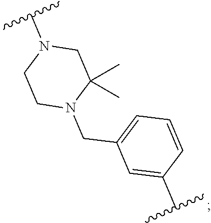 [SMILES: *c1cccc(CN2CCN(*)CC2(C)C)c1]

wherein the left attachment point connects to LBM and the right attachment point connects to the —X— group of Formula A-I or —S(O)₂— group of Formula I. In an embodiment, L is

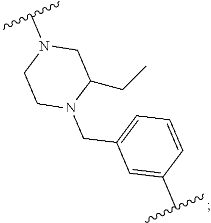 [SMILES: *c1cccc(CN2CCN(*)CC2CC)c1]

wherein the left attachment point connects to LBM and the right attachment point connects to the —X— group of Formula A-I or —S(O)₂— group of Formula I. In an embodiment, L is

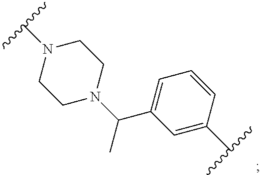 [SMILES: *c1cccc(C(C)N2CCN(*)CC2)c1]

wherein the left attachment point connects to LBM and the right attachment point connects to the —X— group of Formula A-I or —S(O)₂— group of Formula I. In an embodiment, L is

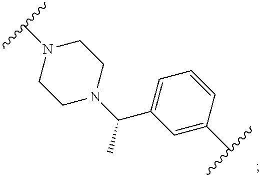 [SMILES: *c1cccc([C@H](C)N2CCN(*)CC2)c1]

wherein the left attachment point connects to LBM and the right attachment point connects to the —X— group of Formula A-I or —S(O)₂— group of Formula I. In an embodiment, L is

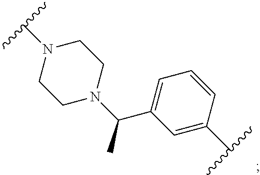 [SMILES: *c1cccc([C@@H](C)N2CCN(*)CC2)c1]

wherein the left attachment point connects to LBM and the right attachment point connects to the —X— group of Formula A-I or —S(O)₂— group of Formula I. In an embodiment, L is

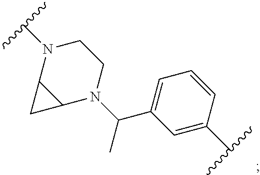 [SMILES: *c1cccc(C(C)N2CCN(*)C3CC32)c1]

wherein the left attachment point connects to LBM and the right attachment point connects to the —X— group of Formula A-I or —S(O)₂— group of Formula I. In an embodiment, L is

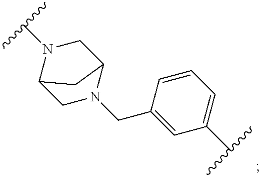 [SMILES: *c1cccc(CN2CC3CC2CN3*)c1]

wherein the left attachment point connects to LBM and the right attachment point connects to the —X— group of Formula A-I or —S(O)₂— group of Formula I. In an embodiment, L is

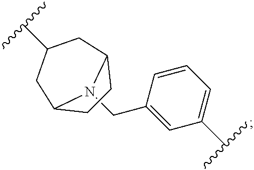 [SMILES: *c1cccc(CN2C3CCC2CC(*)C3)c1]

wherein the left attachment point connects to LBM and the right attachment point connects to the —X— group of Formula A-I or —S(O)₂— group of Formula I. In an embodiment, L is

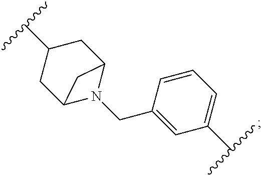 [SMILES: *c1cccc(CN2C3CC(*)CC2C3)c1]

wherein the left attachment point connects to LBM and the right attachment point connects to the —X— group of Formula A-I or —S(O)₂— group of Formula I. In an embodiment, L is

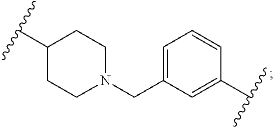 [SMILES: *c1cccc(CN2CCC(*)CC2)c1]

wherein the left attachment point connects to LBM and the right attachment point connects to the —X— group of Formula A-I or —S(O)₂— group of Formula I. In an embodiment, L is

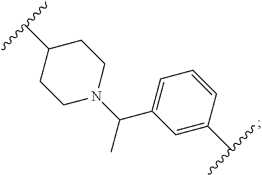 [SMILES: *c1cccc(C(C)N2CCC(*)CC2)c1]

wherein the left attachment point connects to LBM and the right attachment point connects to the —X— group of Formula A-I or —S(O)₂— group of Formula I. In an embodiment, L is

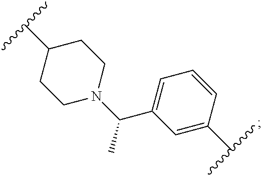 [SMILES: *c1cccc([C@H](C)N2CCC(*)CC2)c1]

wherein the left attachment point connects to LBM and the right attachment point connects to the —X— group of Formula A-I or —S(O)₂— group of Formula I. In an embodiment, L is

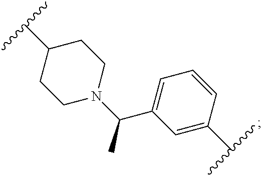 [SMILES: *c1cccc([C@@H](C)N2CCC(*)CC2)c1]

wherein the left attachment point connects to LBM and the right attachment point connects to the —X— group of Formula A-I or —S(O)₂— group of Formula I. In an embodiment, L is

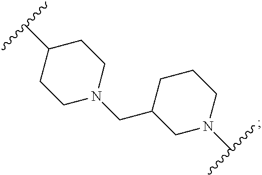 [SMILES: *C1CCN(CC2CCCN(*)C2)CC1]

wherein the left attachment point connects to LBM and the right attachment point connects to the —X— group of Formula A-I or —S(O)₂— group of Formula I. In an embodiment, L is

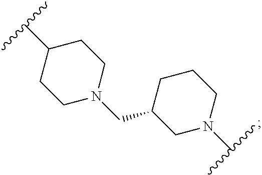 [SMILES: *C1CCN(C[C@@H]2CCCN(*)C2)CC1]

wherein the left attachment point connects to LBM and the right attachment point connects to the —X— group of Formula A-I or —S(O)₂— group of Formula I. In an embodiment, L is

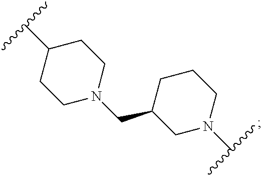 [SMILES: *C1CCN(C[C@H]2CCCN(*)C2)CC1]

wherein the left attachment point connects to LBM and the right attachment point connects to the —X— group of Formula A-I or —S(O)₂— group of Formula I. In an embodiment, L is

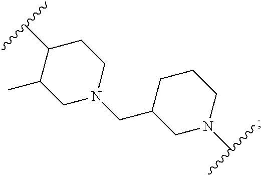 [SMILES: *C1CCN(CC2CCCN(*)C2)CC1C]

wherein the left attachment point connects to LBM and the right attachment point connects to the —X— group of Formula A-I or —S(O)₂— group of Formula I. In an embodiment, L is

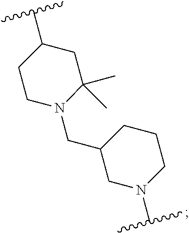 [SMILES: *C1CCN(CC2CCCN(*)C2)C(C)(C)C1]

wherein the left attachment point connects to LBM and the right attachment point connects to the —X— group of Formula A-I or —S(O)₂— group of Formula I. In an embodiment, L is

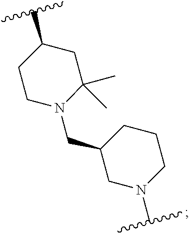 [SMILES: *[C@@H]1CCN(C[C@H]2CCCN(*)C2)C(C)(C)C1]

wherein the left attachment point connects to LBM and the right attachment point connects to the —X— group of Formula A-I or —S(O)₂— group of Formula I. In an embodiment, L is

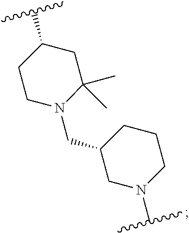 [SMILES: *[C@H]1CCN(C[C@@H]2CCCN(*)C2)C(C)(C)C1]

wherein the left attachment point connects to LBM and the right attachment point connects to the —X— group of Formula A-I or —S(O)₂— group of Formula I. In an embodiment, L is

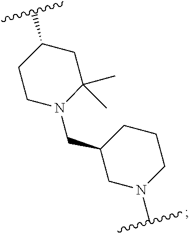 [SMILES: *[C@H]1CCN(C[C@H]2CCCN(*)C2)C(C)(C)C1]

wherein the left attachment point connects to LBM and the right attachment point connects to the —X— group of Formula A-I or —S(O)₂— group of Formula I. In an embodiment, L is

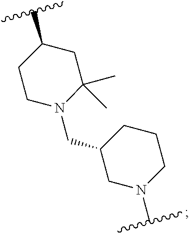 [SMILES: *[C@@H]1CCN(C[C@@H]2CCCN(*)C2)C(C)(C)C1]

wherein the left attachment point connects to LBM and the right attachment point connects to the —X— group of Formula A-I or —S(O)₂— group of Formula I. In an embodiment, L is

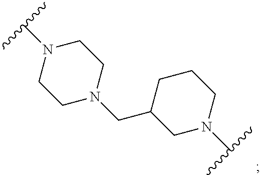 [SMILES: *N1CCN(CC2CCCN(*)C2)CC1]

wherein the left attachment point connects to LBM and the right attachment point connects to the —X— group of Formula A-I or —S(O)₂— group of Formula I. In an embodiment, L is

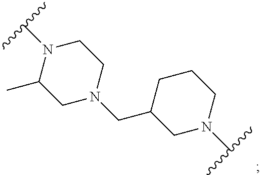 [SMILES: *N1CCCC(CN2CCN(*)C(C)C2)C1]

wherein the left attachment point connects to LBM and the right attachment point connects to the —X— group of Formula A-I or —S(O)₂— group of Formula I. In an embodiment, L is

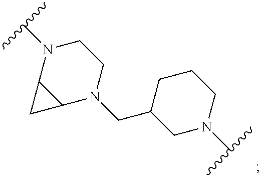 [SMILES: *N1CCCC(CN2CCN(*)C3CC32)C1]

wherein the left attachment point connects to LBM and the right attachment point connects to the —X— group of Formula A-I or —S(O)₂— group of Formula I. In an embodiment, L is

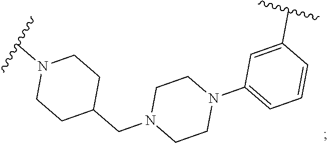 [SMILES: *c1cccc(N2CCN(CC3CCN(*)CC3)CC2)c1]

wherein the left attachment point connects to LBM and the right attachment point connects to the —X— group of Formula A-I or —S(O)₂— group of Formula I. In an embodiment, L is

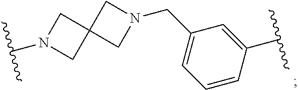 [SMILES: *c1cccc(CN2CC3(CN(*)C3)C2)c1]

wherein the left attachment point connects to LBM and the right attachment point connects to the —X— group of Formula A-I or —S(O)₂— group of Formula I. In an embodiment, L is

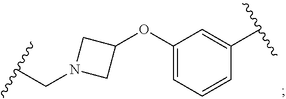 [SMILES: *CN1CC(Oc2cccc(*)c2)C1]

wherein the left attachment point connects to LBM and the right attachment point connects to the —X— group of Formula A-I or —S(O)₂— group of Formula I. In an embodiment, L is

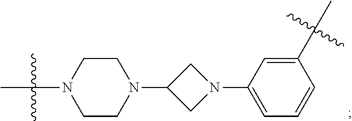 [SMILES: *c1cccc(N2CC(N3CCN(*)CC3)C2)c1]

wherein the left attachment point connects to LBM and the right attachment point connects to the —X— group of Formula A-I or —S(O)₂— group of Formula I. In an embodiment, L is

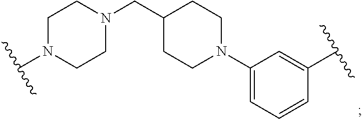 [SMILES: *c1cccc(N2CCC(CN3CCN(*)CC3)CC2)c1]

wherein the left attachment point connects to LBM and the right attachment point connects to the —X— group of Formula A-I or —S(O)₂— group of Formula I. In an embodiment, L is

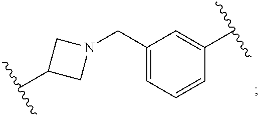 [SMILES: *c1cccc(CN2CC(*)C2)c1]

wherein the left attachment point connects to LBM and the right attachment point connects to the —X— group of Formula A-I or —S(O)₂— group of Formula I. In an embodiment, L is

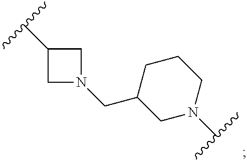 [SMILES: *C1CN(CC2CCCN(*)C2)C1]

wherein the left attachment point connects to LBM and the right attachment point connects to the —X— group of Formula A-I or —S(O)₂— group of Formula I. In an embodiment, L is

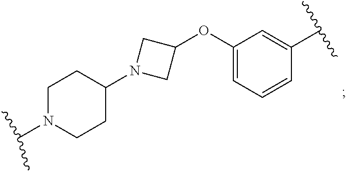 [SMILES: *c1cccc(OC2CN(C3CCN(*)CC3)C2)c1]

wherein the left attachment point connects to LBM and the right attachment point connects to the —X— group of Formula A-I or —S(O)₂— group of Formula I. In an embodiment, L is

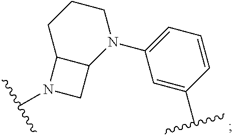 [SMILES: *c1cccc(N2CCCC3C2CN3*)c1]

wherein the left attachment point connects to LBM and the right attachment point connects to the —X— group of Formula A-I or —S(O)₂— group of Formula I. In an embodiment, L is

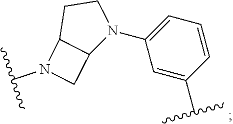 [SMILES: *c1cccc(N2CCC3C2CN3*)c1]

wherein the left attachment point connects to LBM and the right attachment point connects to the —X— group of Formula A-I or —S(O)₂— group of Formula I. In an embodiment, L is

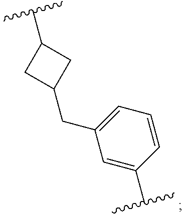 [SMILES: *c1cccc(CC2CC(*)C2)c1]

wherein the left attachment point connects to LBM and the right attachment point connects to the —X— group of Formula A-I or —S(O)₂— group of Formula I. In an embodiment, L is

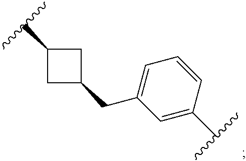 [SMILES: *c1cccc(C[C@H]2C[C@@H](*)C2)c1]

wherein the left attachment point connects to LBM and the right attachment point connects to the —X— group of Formula A-I or —S(O)₂— group of Formula I. In an embodiment, L is

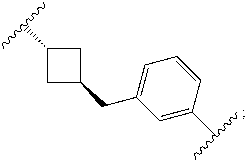 [SMILES: *c1cccc(C[C@H]2C[C@H](*)C2)c1]

wherein the left attachment point connects to LBM and the right attachment point connects to the —X— group of Formula A-I or —S(O)₂— group of Formula I. In an embodiment, L is

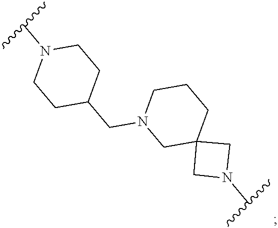 [SMILES: *N1CCC(CN2CCCC3(CN(*)C3)C2)CC1]

wherein the left attachment point connects to LBM and the right attachment point connects to the —X— group of Formula A-I or —S(O)₂— group of Formula I. In an embodiment, L is

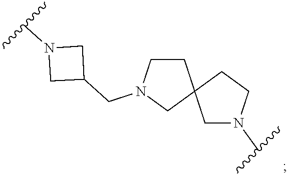 [SMILES: *N1CC(CN2CCC3(CCN(*)C3)C2)C1]

wherein the left attachment point connects to LBM and the right attachment point connects to the —X— group of Formula A-I or —S(O)₂— group of Formula I. In an embodiment, L is

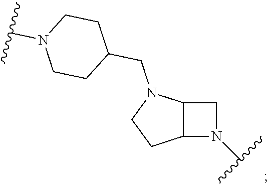 [SMILES: *N1CCC(CN2CCC3C2CN3*)CC1]

wherein the left attachment point connects to LBM and the right attachment point connects to the —X— group of Formula A-I or —S(O)₂— group of Formula I. In an embodiment, L is

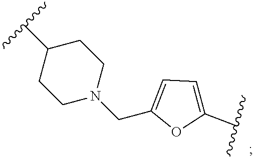 [SMILES: *c1ccc(CN2CCC(*)CC2)o1]

wherein the left attachment point connects to LBM and the right attachment point connects to the —X— group of Formula A-I or —S(O)₂— group of Formula I. In an embodiment, L is

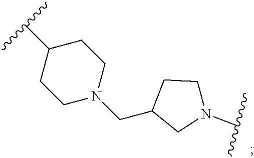 [SMILES: *C1CCN(CC2CCN(*)C2)CC1]

wherein the left attachment point connects to LBM and the right attachment point connects to the —X— group of Formula A-I or —S(O)₂— group of Formula I. In an embodiment, L is

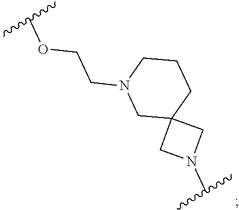 [SMILES: *OCCN1CCCC2(CN(*)C2)C1]

wherein the left attachment point connects to LBM and the right attachment point connects to the —X— group of Formula A-I or —S(O)₂— group of Formula I. In an embodiment, L is

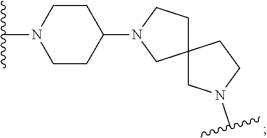 [SMILES: *N1CCC(N2CCC3(CCN(*)C3)C2)CC1]

wherein the left attachment point connects to LBM and the right attachment point connects to the —X— group of Formula A-I or —S(O)₂— group of Formula I. In an embodiment, L is

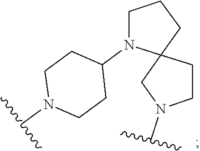 [SMILES: *N1CCC(N2CCCC23CCN(*)C3)CC1]

wherein the left attachment point connects to LBM and the right attachment point connects to the —X— group of Formula A-I or —S(O)₂— group of Formula I. In an embodiment, L is

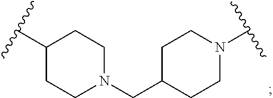 [SMILES: *C1CCN(CC2CCN(*)CC2)CC1]

wherein the left attachment point connects to LBM and the right attachment point connects to the —X— group of Formula A-I or —S(O)₂— group of Formula I. In an embodiment, L is

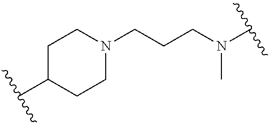 [SMILES: *C1CCN(CCCN(*)C)CC1]

wherein the left attachment point connects to LBM and the right attachment point connects to the —X— group of Formula A-I or —S(O)₂— group of Formula I.

In an embodiment, L is

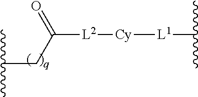

wherein L¹, —Cy—, L² and q are as defined in any of the embodiments described herein, and wherein the left attachment point connects to LBM and the right attachment point connects to the —S(O)₂— group of Formula I or the X group of Formula A-I.

In an embodiment,

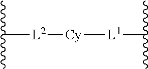

is selected from

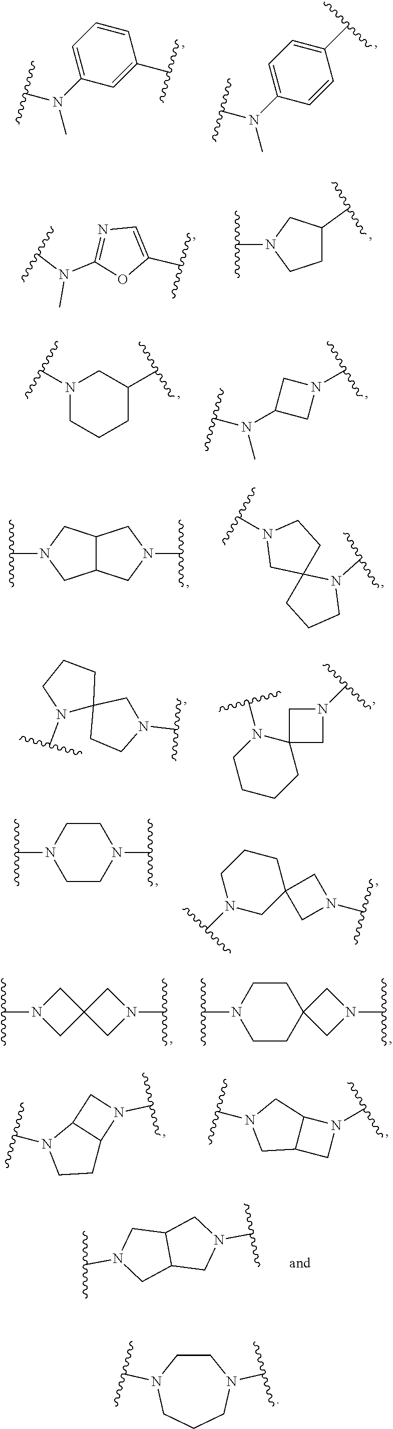

wherein the attachment point to the left connects to the carbonyl, and the attachment point to the right connects to the sulfonyl of the Compounds of Formula I or the X group of Formula A-I.

In an embodiment,

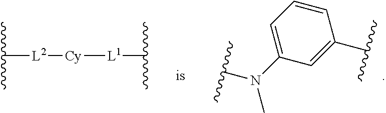

In an embodiment,

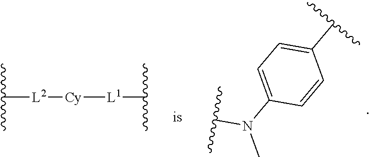

In an embodiment,

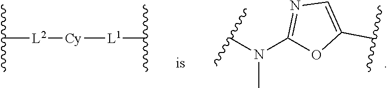

In an embodiment,

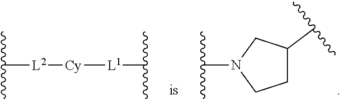

In an embodiment,

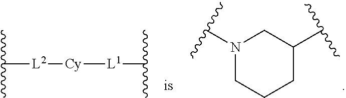

In an embodiment,

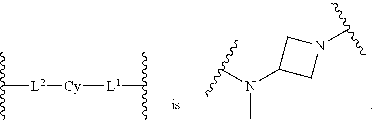

In an embodiment,

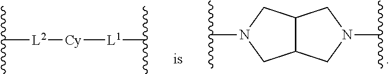

In an embodiment,

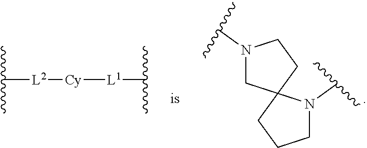

In an embodiment,

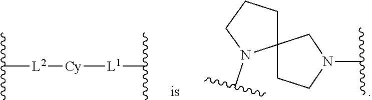

In an embodiment,

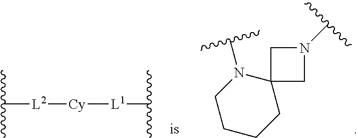

In an embodiment,

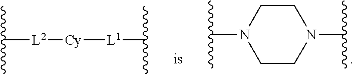

In an embodiment,

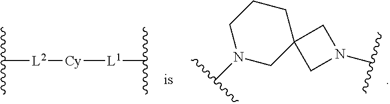

In an embodiment,

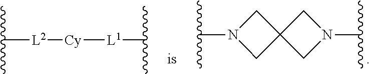

In an embodiment,

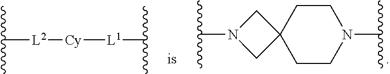

In an embodiment,

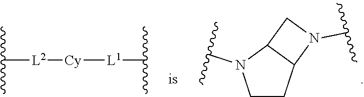

In an embodiment,

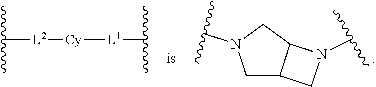

In an embodiment,

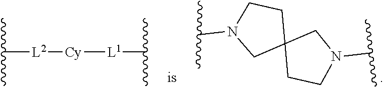

In an embodiment,

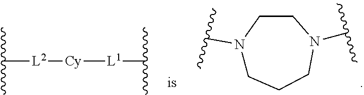

In an embodiment, L is selected from

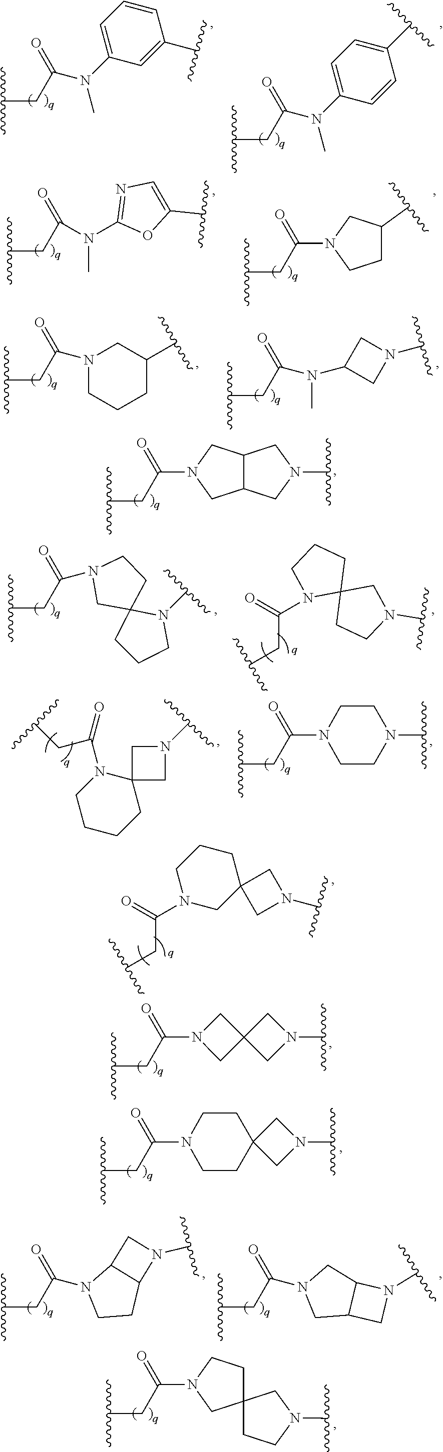

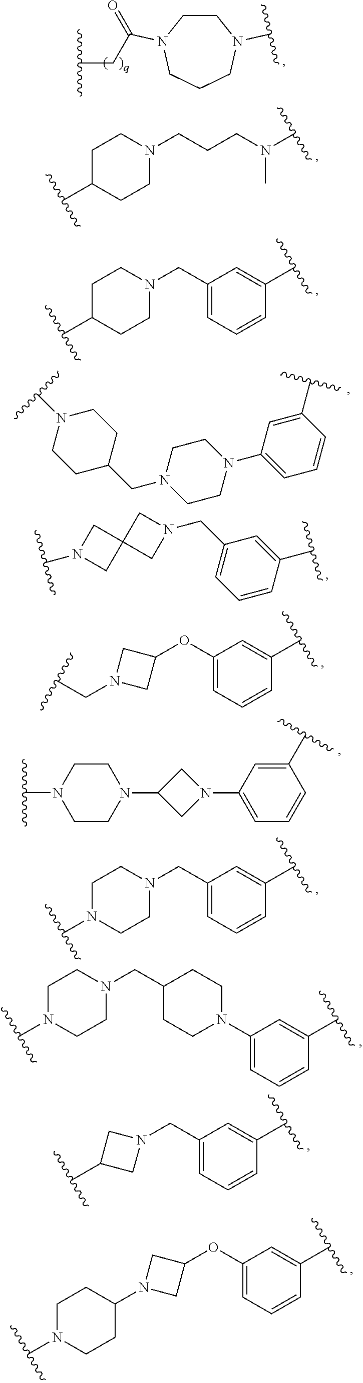

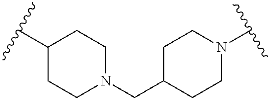 [SMILES: *C1CCN(CC2CCN(*)CC2)CC1]

wherein the left attachment point connects to LBM and the right attachment point connects to the —S(O)₂— group of Formula I or the X group of Formula A-I and wherein q is as defined in any of the embodiments described herein.

In an embodiment, L is selected from

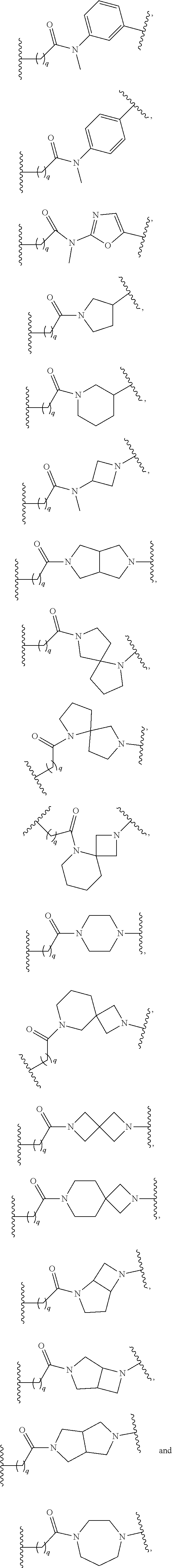

wherein the left attachment point connects to LBM and the right attachment point connects to the —S(O)₂— group of Formula I or the X group of Formula A-I and wherein q is as defined in any of the embodiments described herein.

In an embodiment, L is

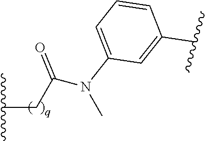

wherein q is as defined in any of the embodiments described herein. In an embodiment, L is

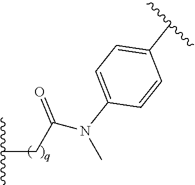

wherein q is as defined in any of the embodiments described herein. In an embodiment, L is

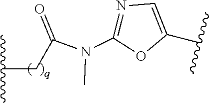

wherein q is as defined in any of the embodiments described herein. In an embodiment, L is

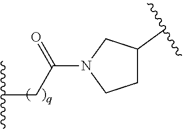

wherein q is as defined in any of the embodiments described herein. In an embodiment, L is

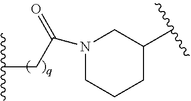

wherein q is as defined in any of the embodiments described herein. In an embodiment, L is

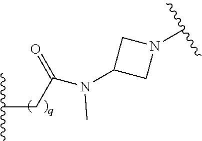

wherein q is as defined in any of the embodiments described herein. In an embodiment, L is

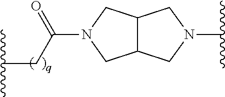

wherein q is as defined in any of the embodiments described herein. In an embodiment, L is

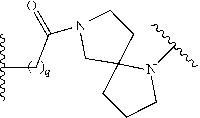

wherein q is as defined in any of the embodiments
described herein. In an embodiment, L is

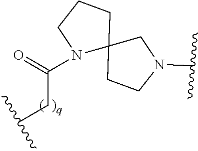

wherein q is as defined in any of the embodiments described herein. In an embodiment, L is

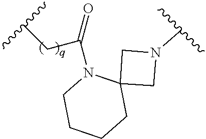

wherein q is as defined in any of the embodiments described herein. In an embodiment, L is

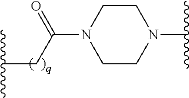

wherein q is as defined in any of the embodiments described herein. In an embodiment, L is

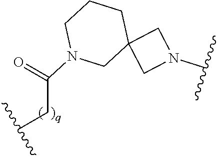

wherein q is as defined in any of the embodiments
described herein. In an embodiment, L is

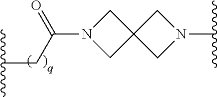

wherein q is as defined in any of the embodiments described herein. In an embodiment, L is

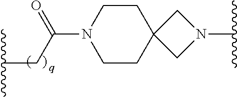

wherein q is as defined in any of the embodiments described herein. In an embodiment, L is

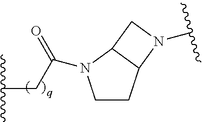

wherein q is as defined in any of the embodiments described herein. In an embodiment L is

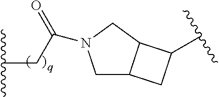

wherein q is as defined in any of the embodiments described herein. In an embodiment, L is

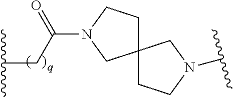

wherein q is as defined in any of the embodiments described herein. In an embodiment, L is

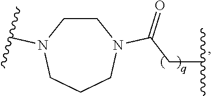

wherein q is as defined in any of the embodiments described herein.

In an embodiment, —L—X— is selected from the group consisting of:

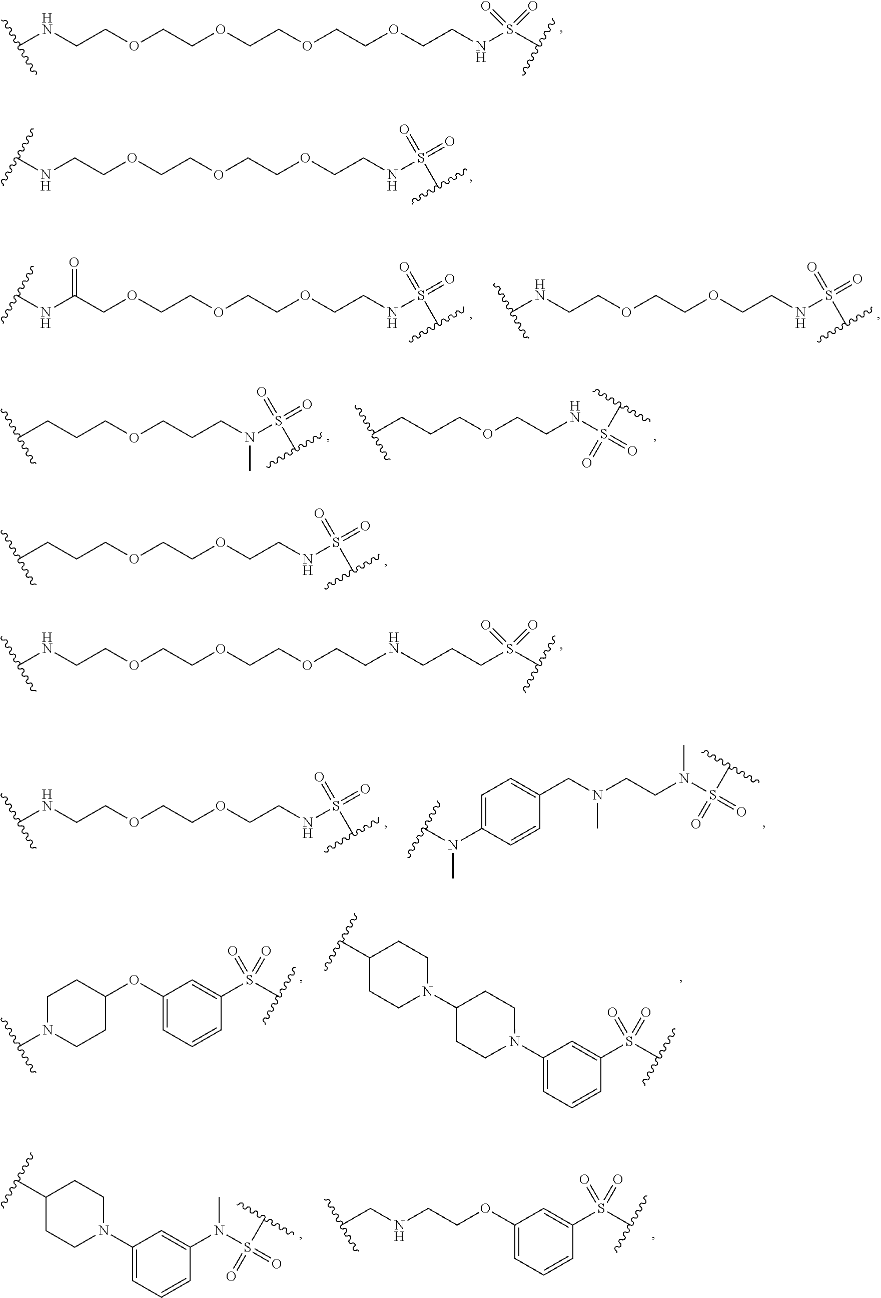

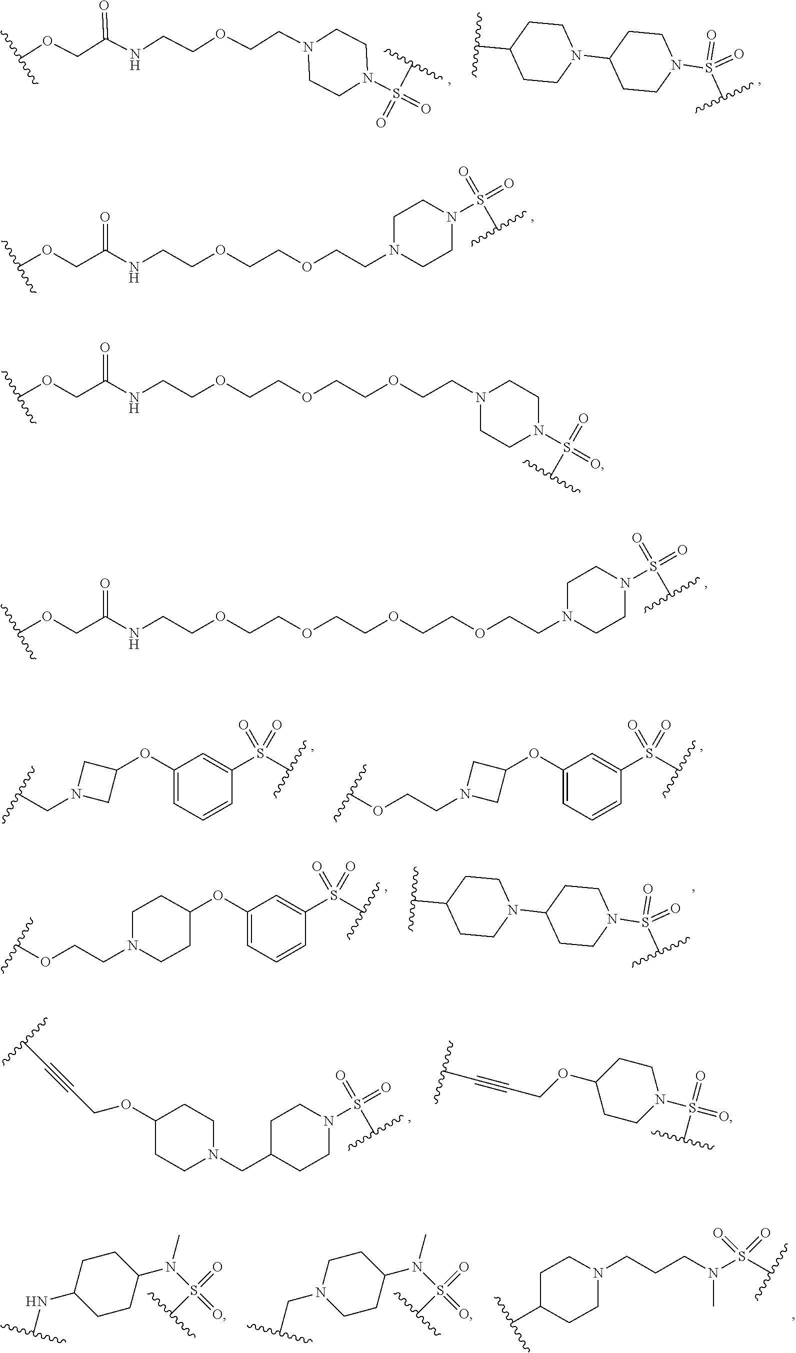

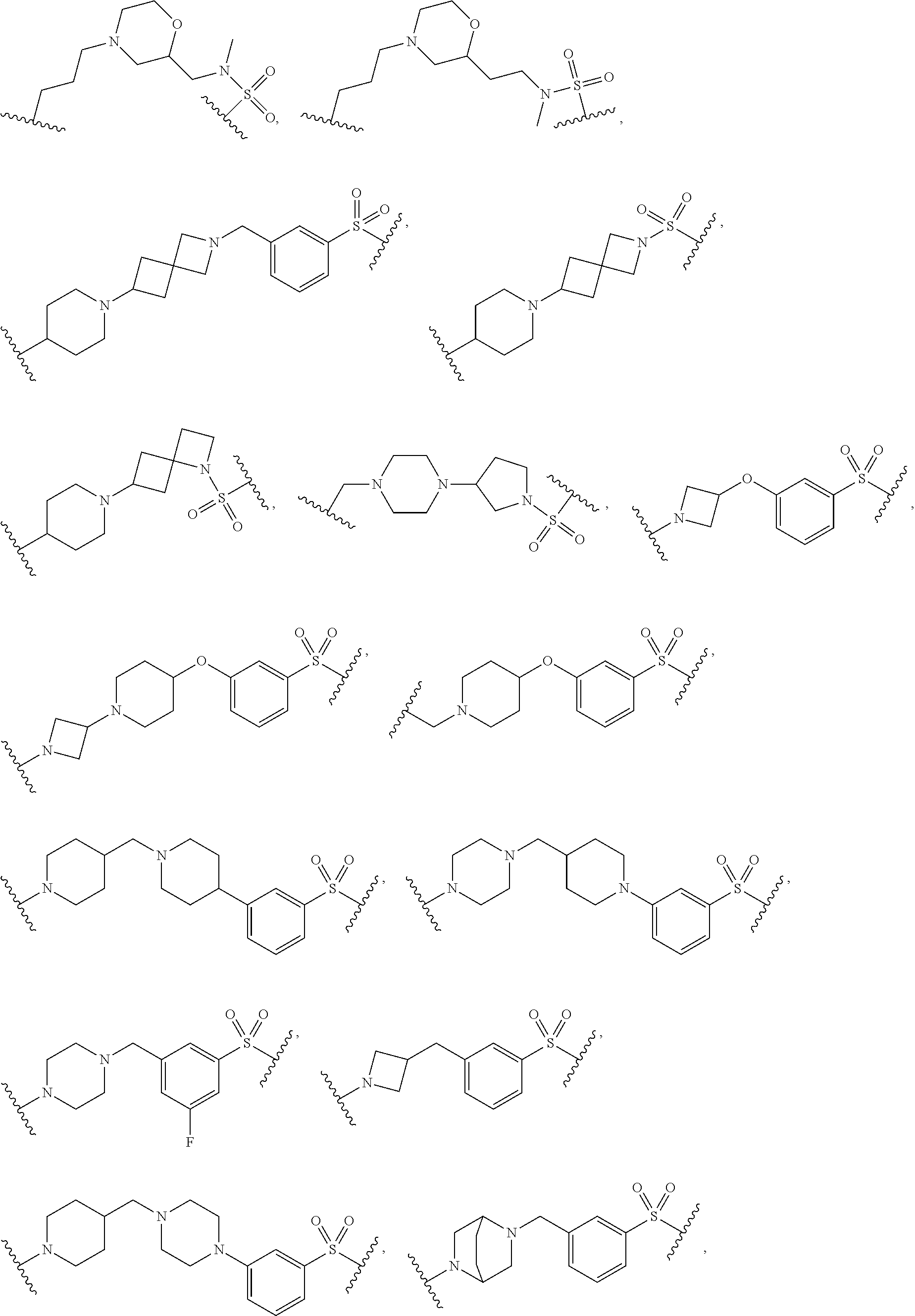

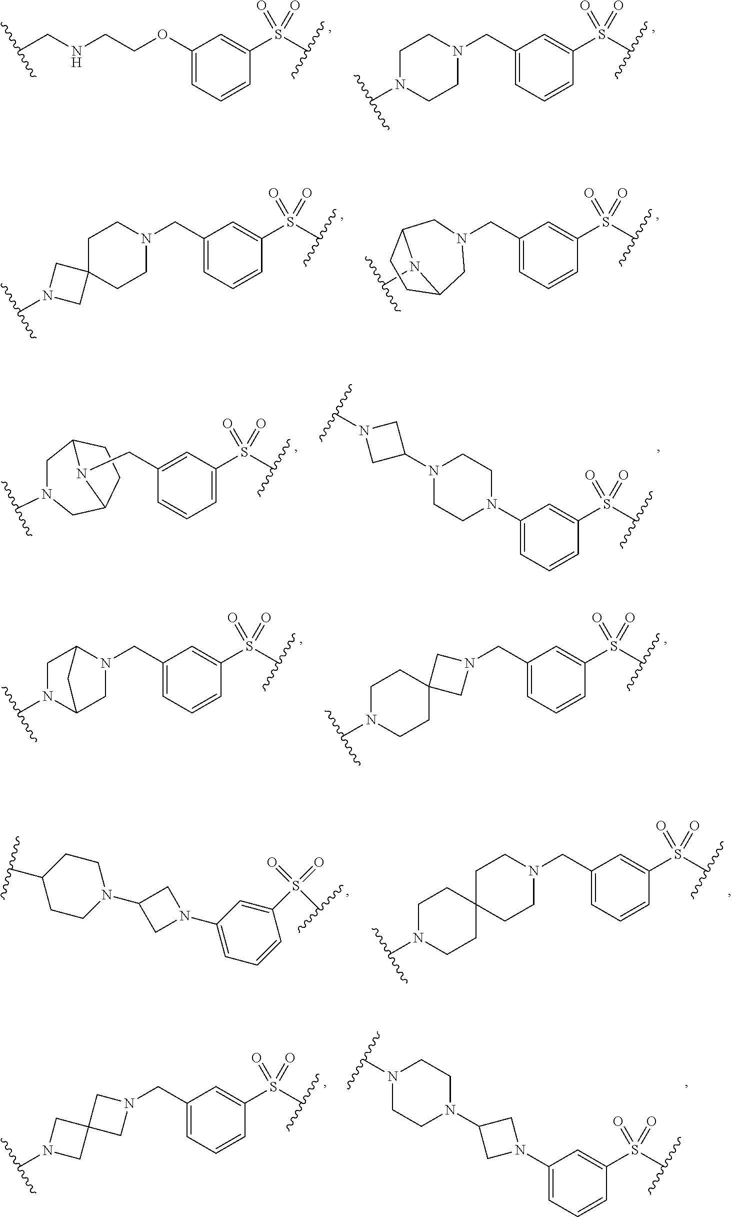

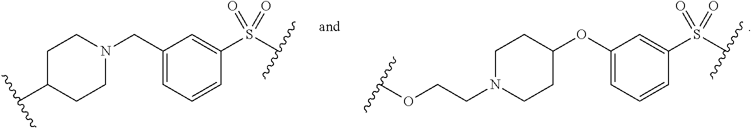 [SMILES: *C1CCN(Cc2cccc(S(*)(=O)=O)c2)CC1 ; *OCCN1CCC(Oc2cccc(S(*)(=O)=O)c2)CC1]

In an embodiment, —L—X— of Formula A-I or L-SO₂— of Formula I is selected from the group consisting of:

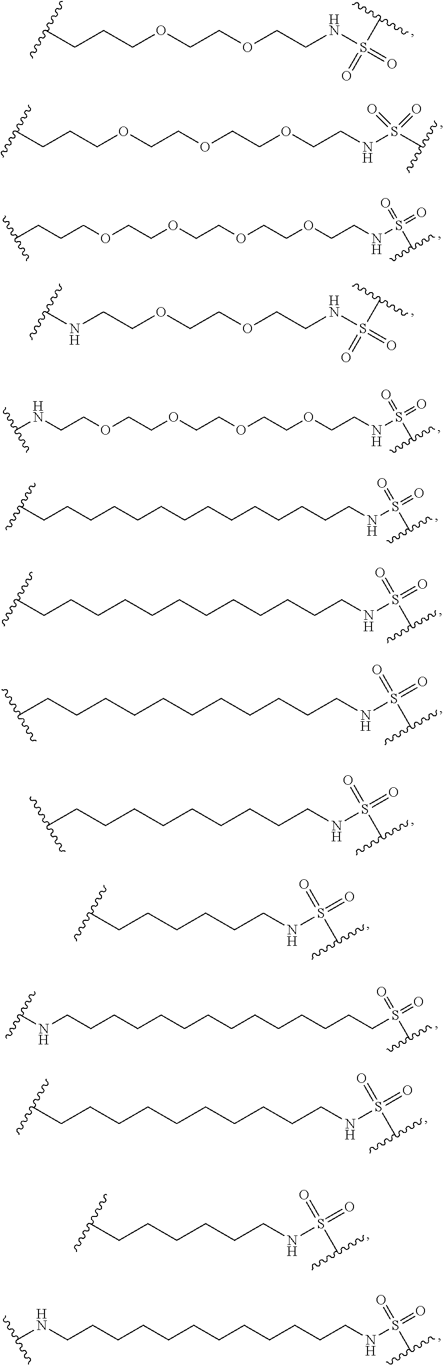

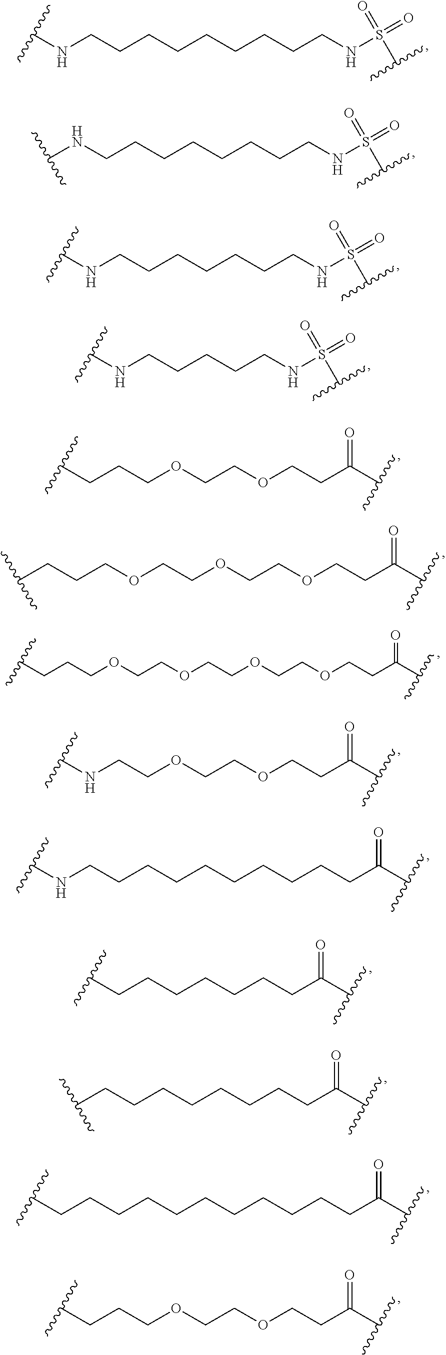

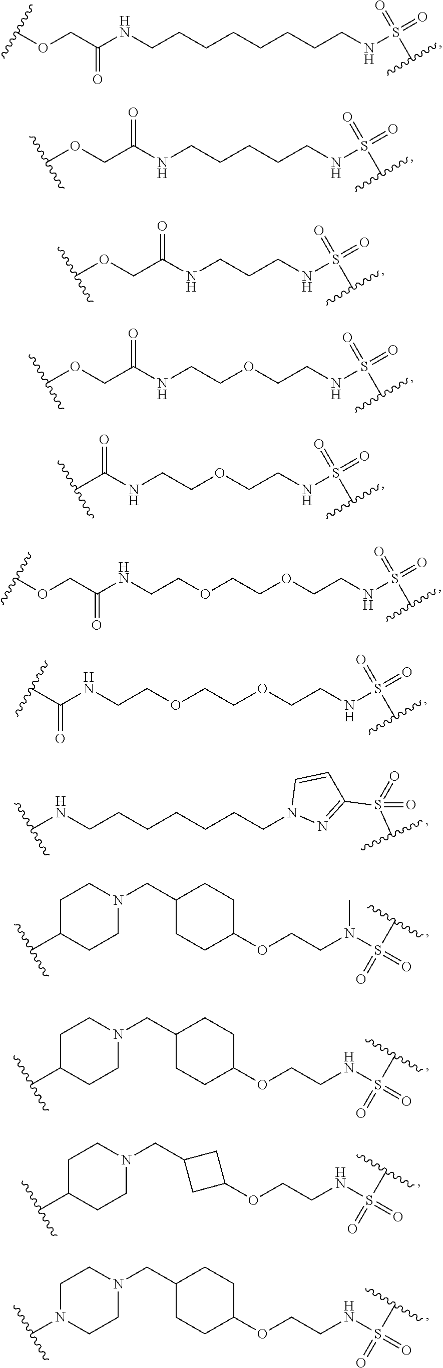

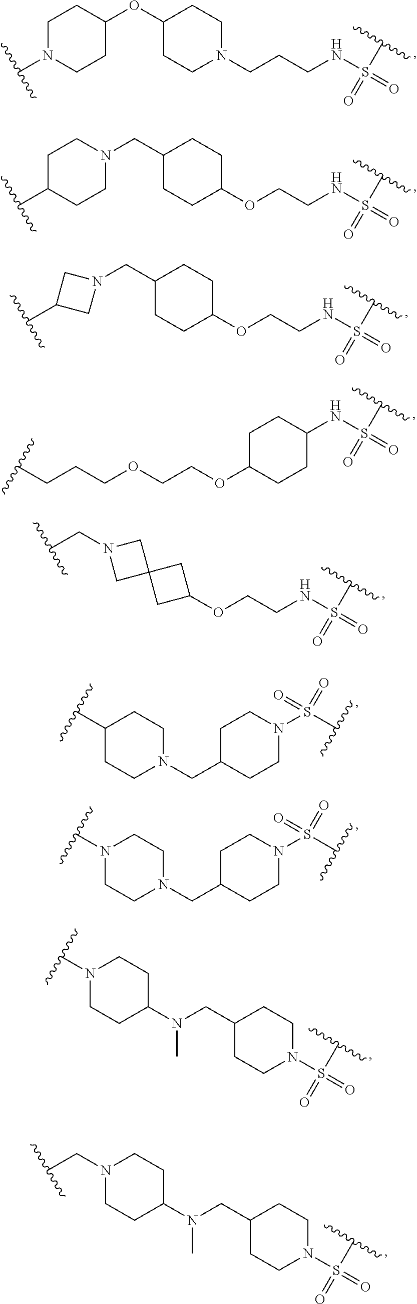

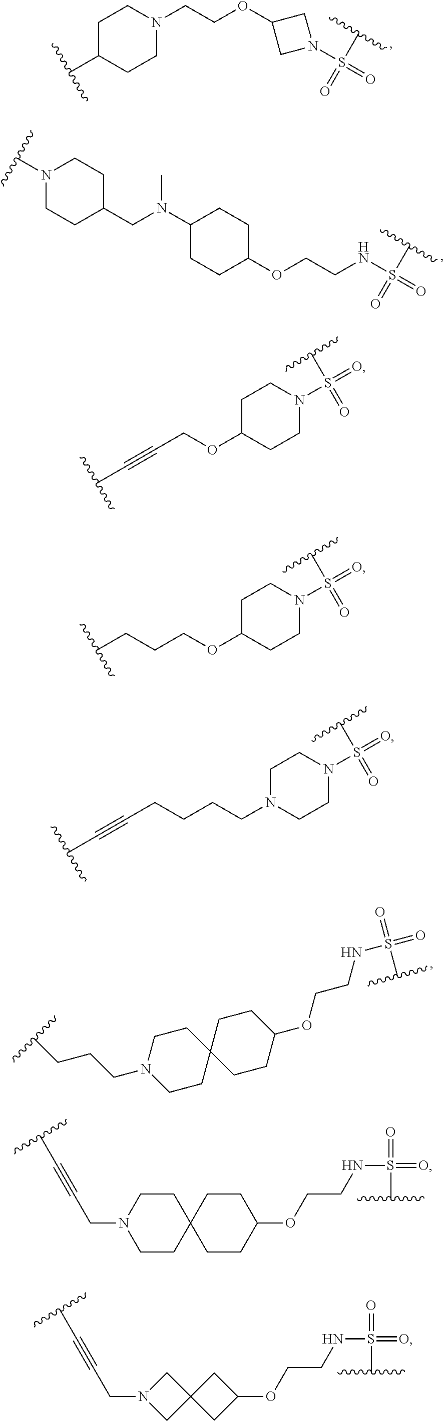

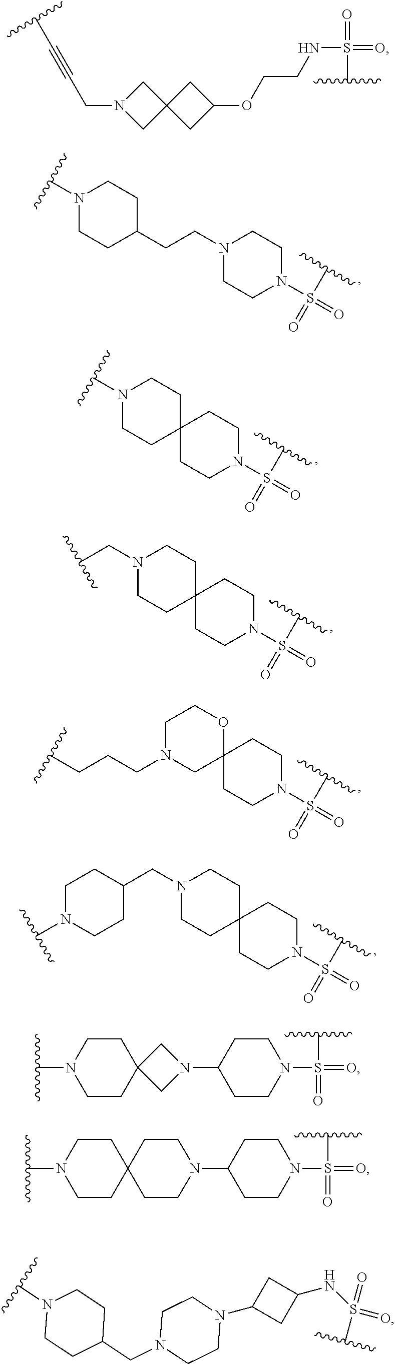

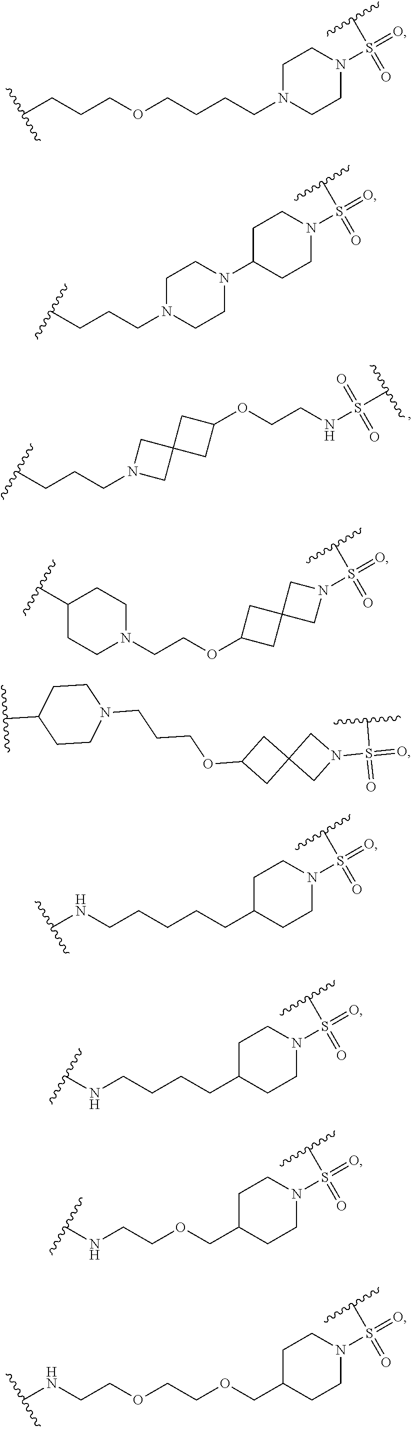

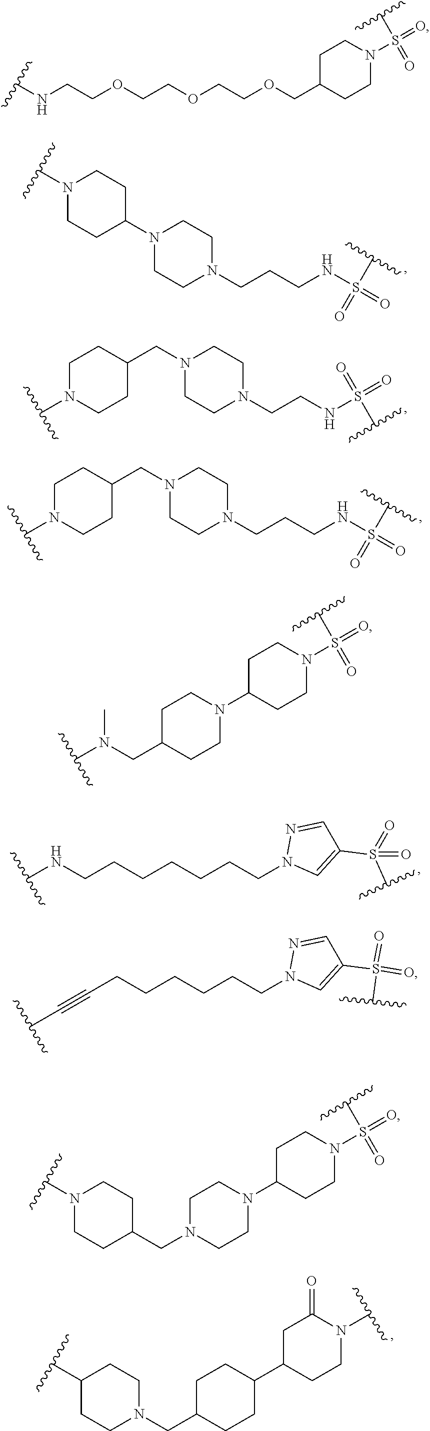

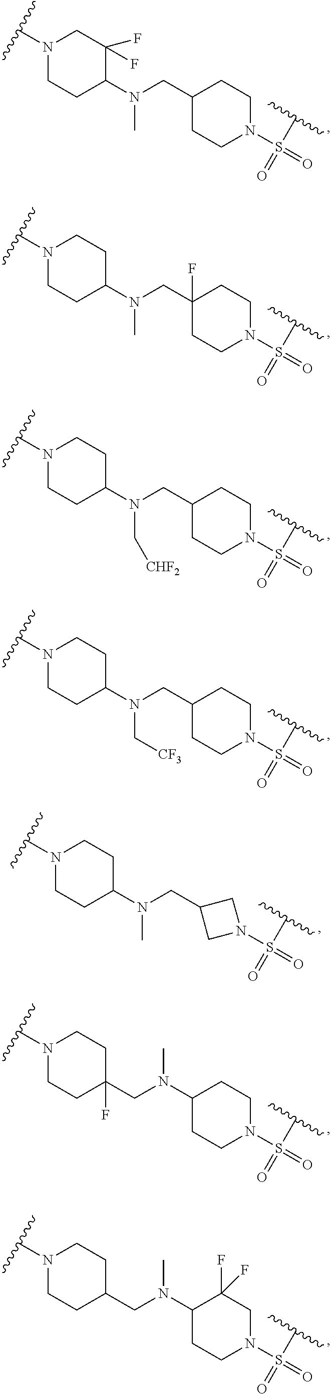

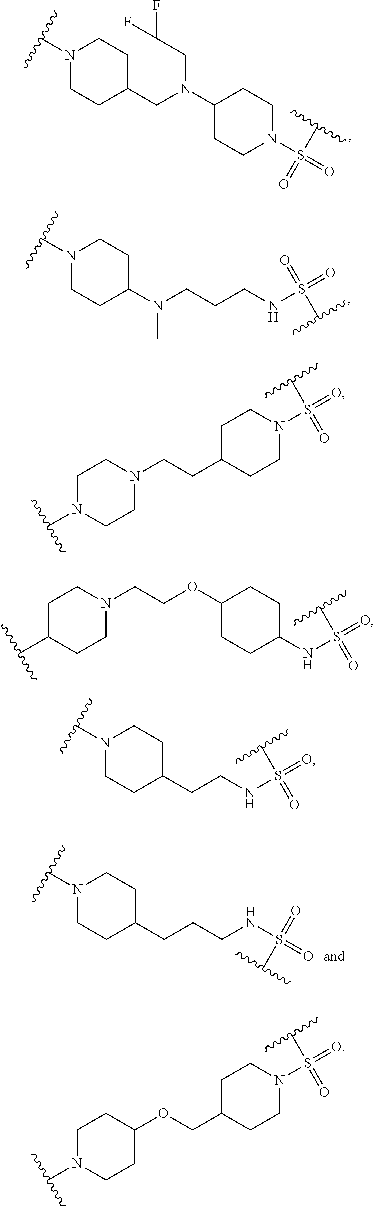

As generally defined herein, each —Cy— is independently a bivalent ring selected from phenylene, an 8-10 membered bicyclic arylene, a 4-7 membered monocyclic carbocyclylene, a 5-11 membered spiro carbocyclylene, a 4-10 membered bicyclic carbocyclylene, a 5-10 membered bridged carbocyclylene, a 4-7 membered monocyclic heterocyclylene having 1-2 heteroatoms independently selected from nitrogen, oxygen, and sulfur, a 5-li membered spiro heterocyclylene having 1-2 heteroatoms independently selected from nitrogen, oxygen, and sulfur, an 4-10 membered bicyclic heterocyclylene having 1-2 heteroatoms independently selected from nitrogen, oxygen, and sulfur, a 5-10 membered bridged bicyclic saturated or partially unsaturated heterocyclylene having 1-2 heteroatoms independently selected from nitrogen, oxygen, and sulfur, a 5-6 membered heteroarylene having 1-4 heteroatoms independently selected from nitrogen, oxygen, and sulfur, or an 8-10 membered bicyclic heteroarylene having 1-5 heteroatoms independently selected from nitrogen, oxygen, or sulfur, wherein each phenylene, arylene, carbocyclylene, heterocyclylene and heteroarylene is substituted with 0, 1, 2, 3, or 4 instances of Re, wherein Re is as defined in any of the embodiments described herein.

In an embodiment, each —Cy— is independently a bivalent ring selected from phenylene, a 4-7 membered monocyclic heterocyclylene having 1-2 heteroatoms independently selected from nitrogen, oxygen, and sulfur, a 5-11 membered spiro heterocyclylene having 1-2 heteroatoms independently selected from nitrogen, oxygen, and sulfur, a 4-10 membered bicyclic heterocyclylene having 1-2 heteroatoms independently selected from nitrogen, oxygen, and sulfur and a 5-6 membered heteroarylene having 1-4 heteroatoms independently selected from nitrogen, oxygen, and sulfur, wherein each phenylene, heterocyclylene and heteroarylene is substituted with 0, 1, 2, 3, or 4 instances of R^{C}, wherein R^{C} is as defined in any of the embodiments described herein.

In an embodiment, each —Cy— is independently a phenylene substituted with 0, 1, 2, 3, or 4 instances of R^{C}, wherein R^{C} is as defined in any of the embodiments described herein. In an embodiment, each —Cy— is independently a 4-7 membered monocyclic heterocyclylene having 1-2 heteroatoms independently selected from nitrogen, oxygen, and sulfur, substituted with 0, 1, 2, 3, or 4 instances of R^{C}, wherein R^{C} is as defined in any of the embodiments described herein. In an embodiment, each —Cy— is a 5-11 membered spiro heterocyclylene having 1-2 heteroatoms independently selected from nitrogen, oxygen, and sulfur, substituted with 0, 1, 2, 3, or 4 instances of R^{C}, wherein R^{C} is as defined in any of the embodiments described herein. In an embodiment, each —Cy— is a 4-10 membered bicyclic heterocyclylene having 1-2 heteroatoms independently selected from nitrogen, oxygen, and sulfur, substituted with 0, 1, 2, 3, or 4 instances of R^{C}, wherein R^{C} is as defined in any of the embodiments described herein. In an embodiment, each —Cy— is a 5-6 membered heteroarylene having 1-4 heteroatoms independently selected from nitrogen, oxygen, and sulfur, substituted with 0, 1, 2, 3, or 4 instances of R^{C}, wherein R^{C} is as defined in any of the embodiments described herein.

In an embodiment, Cy is selected from:

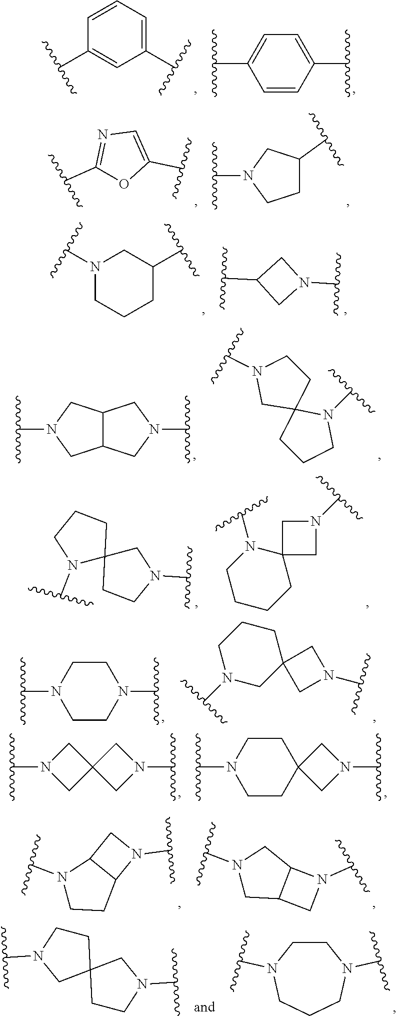

each substituted with 0, 1, 2, 3, or 4 instances of R^{C}, wherein R^{C} is as defined in any of the embodiments described herein. In an embodiment, —Cy— is unsubstituted. In an embodiment, —Cy— is substituted with 0 or 1 instances of R^{C}. In an embodiment, —Cy— is substituted with 0, 1 or 2 instances of R^{C}. In an embodiment, —Cy— is substituted with 0, 1, 2 or 3 instances of R^{C}. In an embodiment, —Cy— is substituted with 1 instance of R^{C}. In an embodiment, —Cy— is substituted with 2 instances of R^{C}. In an embodiment, —Cy— is substituted with 3 instances of R^{C}. In an embodiment, —Cy— is substituted with 4 instances of R^{C}.

As generally defined herein, LBM is selected from:

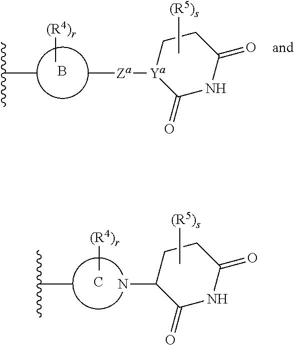

wherein Y^{a}, Z^{a}, R⁴, R⁵ Ring B and Ring C are as defined in any of the embodiments described herein.

In an embodiment, LBM is

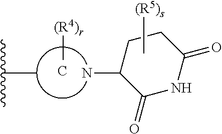

wherein each Ring C, R⁴, R⁵, r and s is as defined in any of the embodiments described herein.

In an embodiment, LBM is

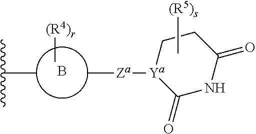

wherein each Ring B, Z^{a}, Y^{a}, R⁴, R⁵, r and s is as defined in any of the embodiments described herein.

In an embodiment, LBM is selected from the group consisting of:

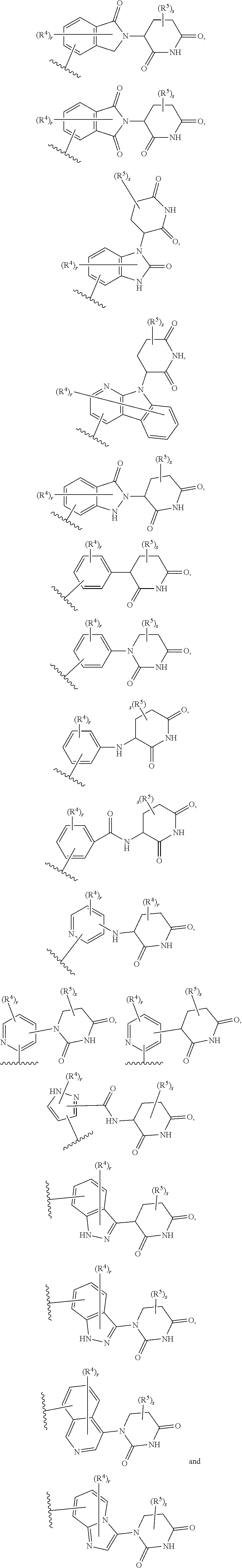

wherein each R⁴, R⁵ r and s is as defined in any of the embodiments described herein.

In an embodiment, LBM is selected from the group consisting of:

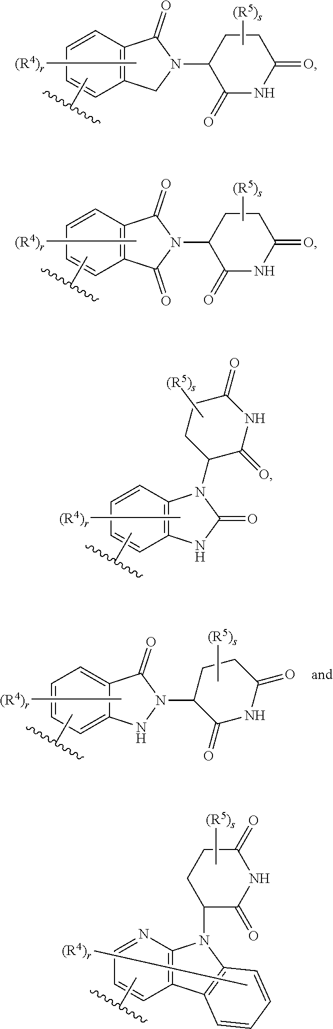

wherein each R⁴, R⁵, r and s is as defined in any of the embodiments described herein.

In an embodiment, LBM is selected from the group consisting of:

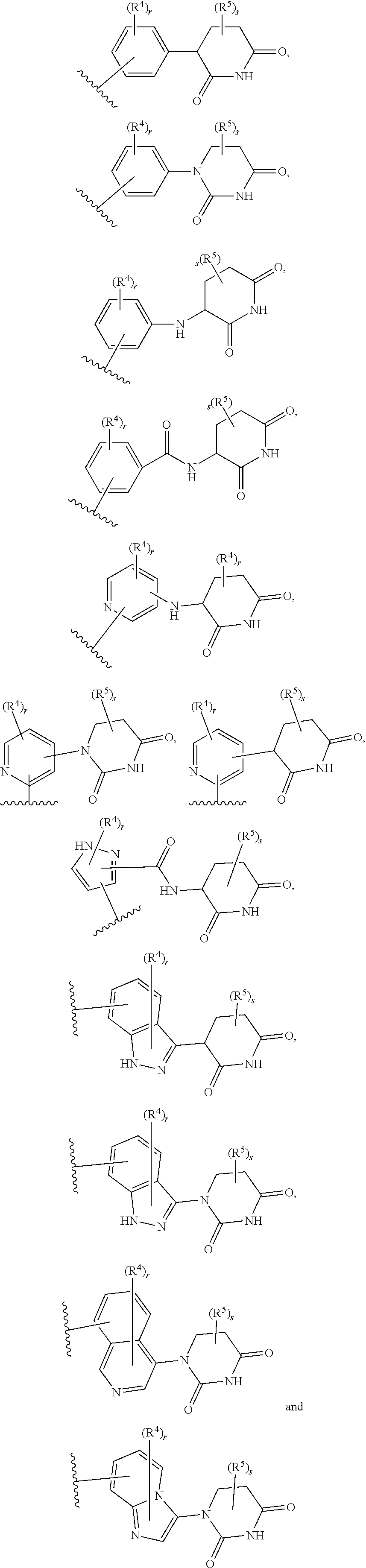

wherein each R⁴, R⁵, r and s is as defined in any of the embodiments described herein.

In an embodiment LBM is selected from

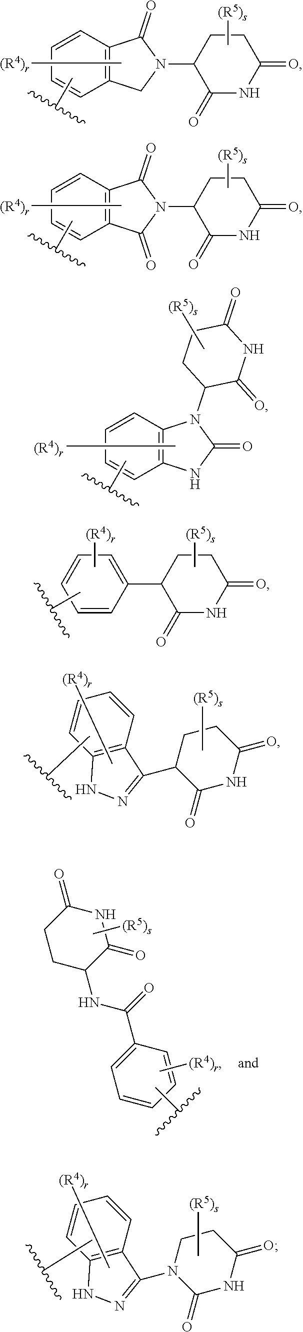

wherein each R⁴, R⁵, r and s is as defined in any of the embodiments described herein.

In an embodiment, the moiety represented by

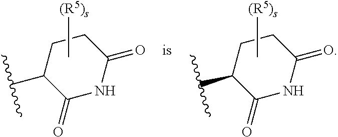

In an embodiment, the moiety represented by

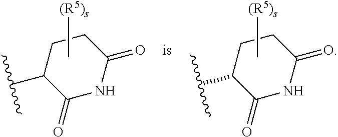

In an embodiment, LBM is

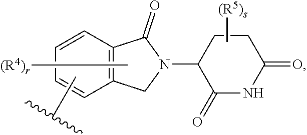

wherein each R⁴, R⁵, r and s is as defined in any of the embodiments described herein. In an embodiment, LBM is

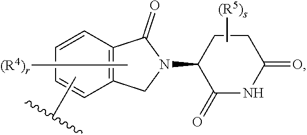

wherein each R⁴, R⁵, r and s is as defined in any of the embodiments described herein. In an embodiment, LBM is

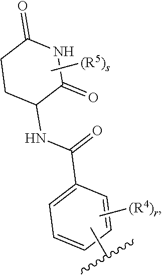

wherein each R⁴, R⁵, r and s is as defined in any of the embodiments described herein. In an embodiment, LBM is

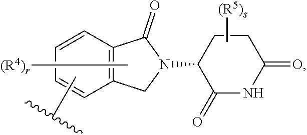

wherein each R⁴, R⁵, r and s is as defined in any of the embodiments described herein. In an embodiment, LBM is

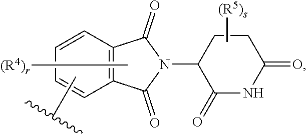

wherein each R⁴, R, r and s is as defined in any of the embodiments described herein. In an embodiment, LBM is

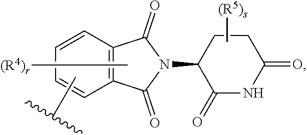

wherein each R⁴, R⁵, r and s is as defined in any of the embodiments described herein. In an embodiment, LBM is

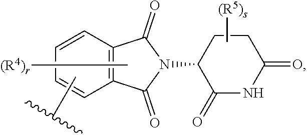

wherein each R⁴, R, r and s is as defined in any of the embodiments described herein. In an embodiment, LBM is

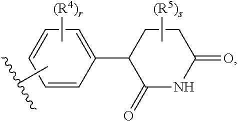

wherein each R⁴, R⁵, r and s is as defined in any of the embodiments described herein. In an embodiment, LBM is

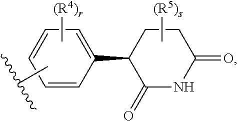

wherein each R⁴, R⁵, r and s is as defined in any of the embodiments described herein. In an embodiment, LBM is

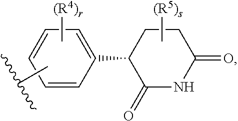

wherein each R⁴, R⁵ r and s is as defined in any of the embodiments described herein. In an embodiment, LBM is

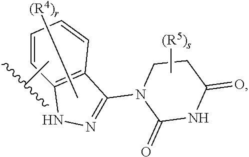

wherein each R⁴, R⁵, r and s is as defined in any of the embodiments described herein. In an embodiment, LBM is

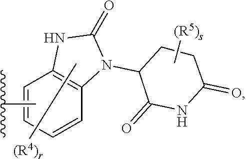

wherein each R⁴, R⁵, r and s is as defined in any of the embodiments described herein. In an embodiment, LBM is

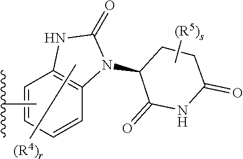

wherein each R⁴, R⁵, r and s is as defined in any of the embodiments described herein. In an embodiment, LBM is

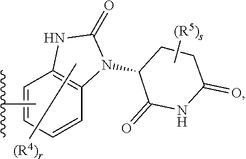

wherein each R⁴, R⁵, r and s is as defined in any of the embodiments described herein.

In an embodiment, LBM is selected from:

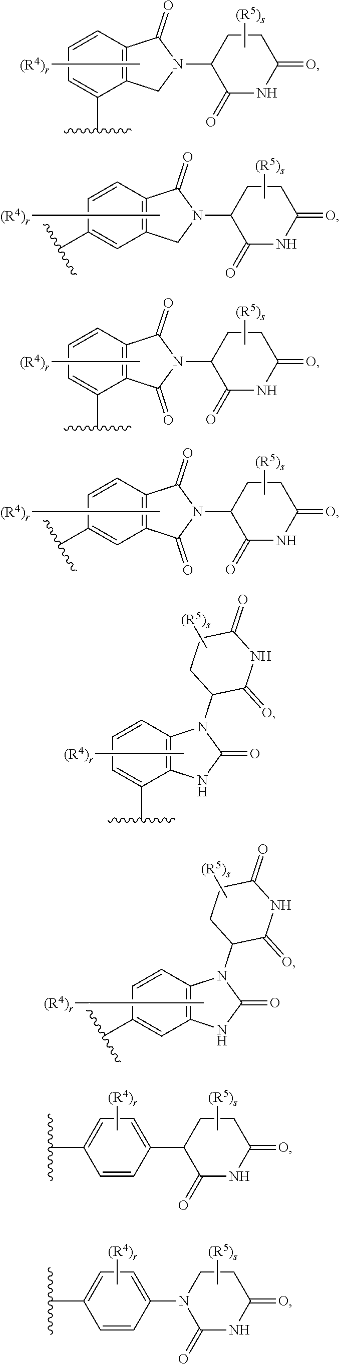

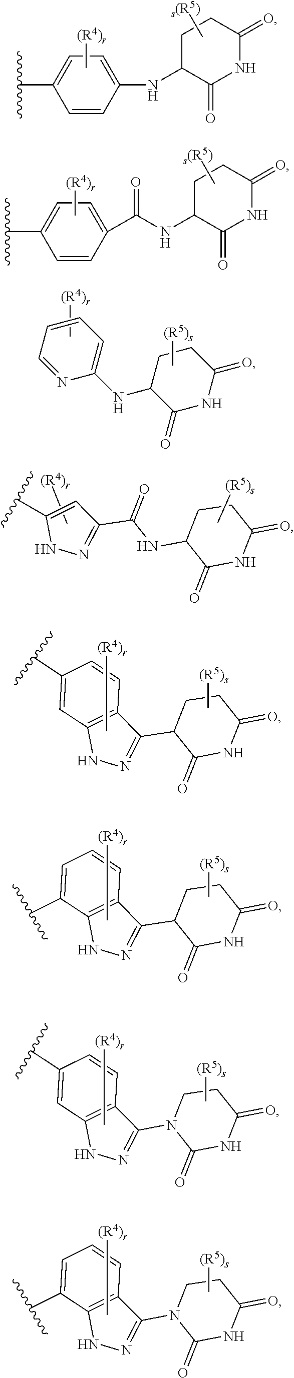

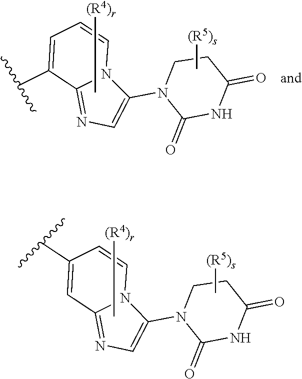

wherein each R⁴, R⁵, r and s is as defined in any of the embodiments described herein.

In an embodiment, LBM is selected from the group consisting of:

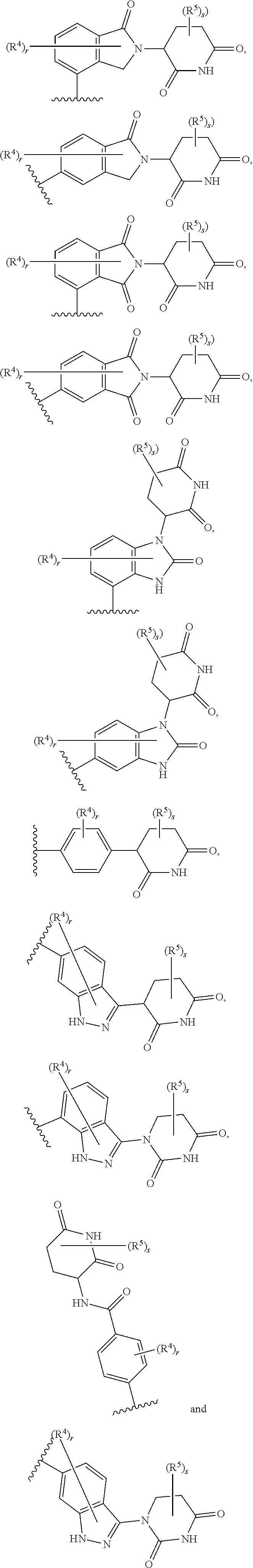

wherein each R⁴, R⁵, r and s is as defined in any of the embodiments described herein.

In an embodiment, LBM is selected from the group consisting of:

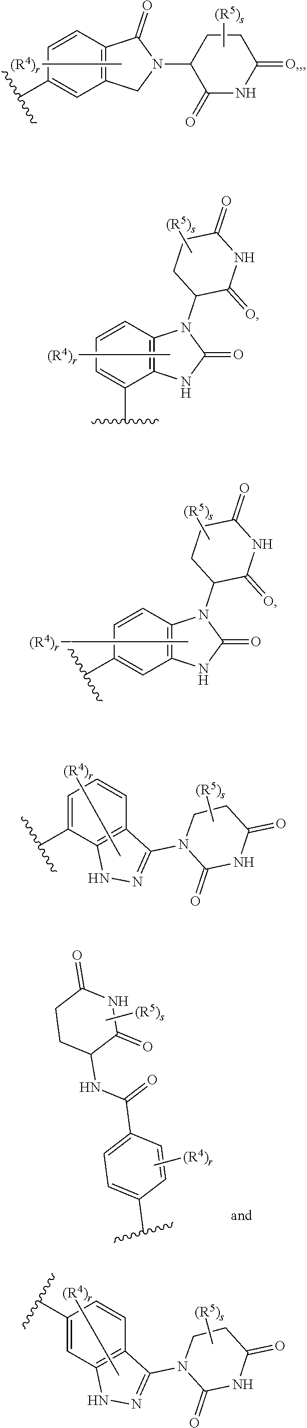

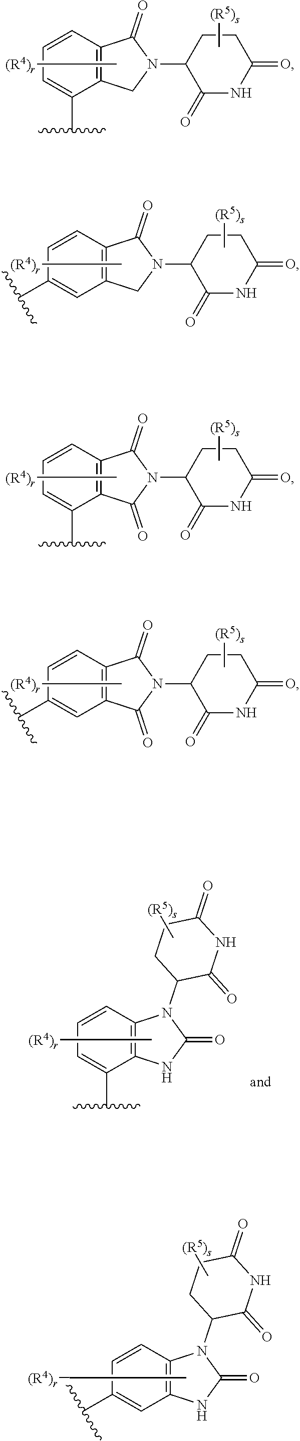

wherein each R⁴, R⁵, r and s is as defined in any of the embodiments described herein.

In an embodiment, LBM is selected from the group consisting of:

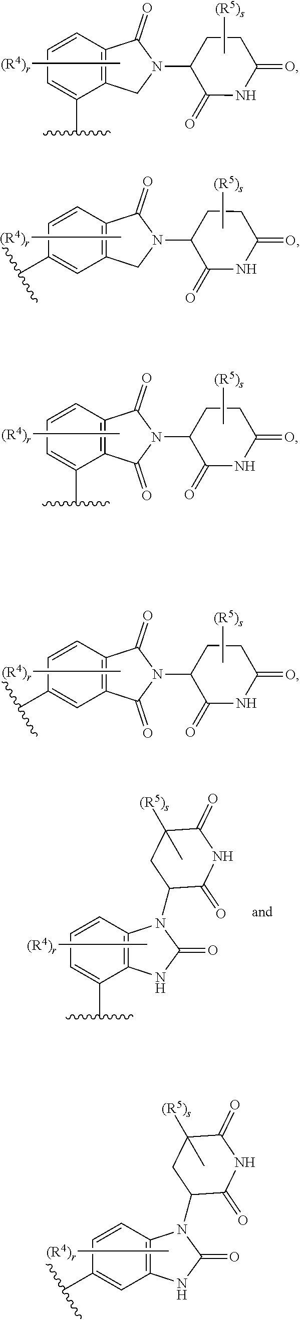

wherein each R⁴, R⁵, r and s is as defined in any of the embodiments described herein.

In an embodiment, LBM is selected from the group consisting of:

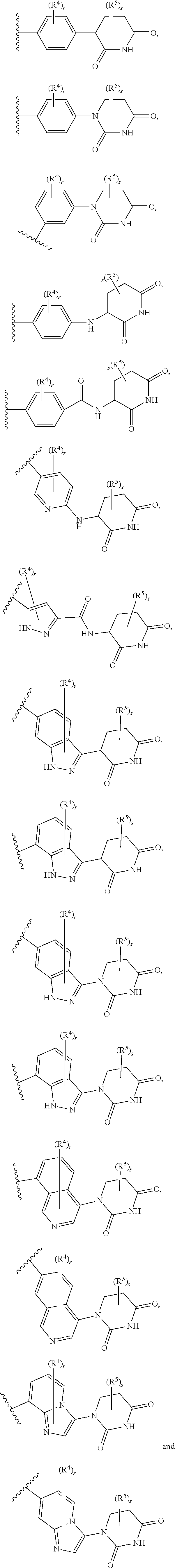

wherein each R⁴, R⁵, r and s is as defined in any of the embodiments described herein.

In an embodiment, LBM is

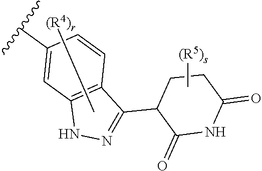

wherein each R⁴, R⁵, r and s is as defined in any of the embodiments described herein. In an embodiment, LBM is

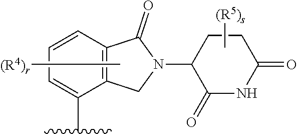

wherein each R⁴, R⁵, r and s is as defined in any of the embodiments described herein. In an embodiment, LBM is

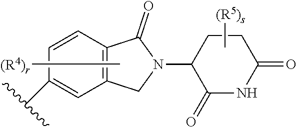

wherein each R⁴, R⁵, r and s is as defined in any of the embodiments described herein. In an embodiment, LBM is

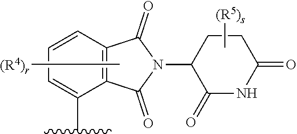

wherein each R⁴, R⁵, r and s is as defined in any of the embodiments described herein. In an embodiment, LBM is

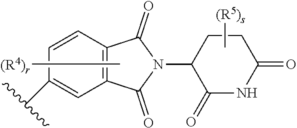

wherein each R⁴, R⁵, r and s is as defined in any of the embodiments described herein. In an embodiment, LBM is

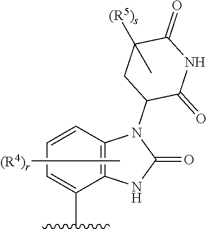

wherein each R⁴, R⁵, r and s is as defined in any of the embodiments described herein. In an embodiment, LBM is

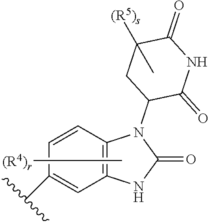

wherein each R⁴, R⁵, r and s is as defined in any of the embodiments described herein. In an embodiment, LBM is

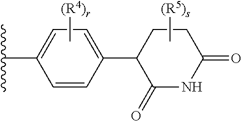

wherein each R⁴, R⁵, r and s is as defined in any of the embodiments described herein. In an embodiment, LBM is

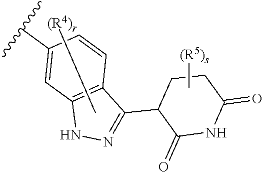

wherein each R⁴, R⁵, r and s is as defined in any of the embodiments described herein. In an embodiment, LBM is

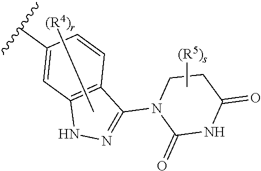

wherein each R⁴, R⁵, r and s is as defined in any of the embodiments described herein. In an embodiment, LBM is

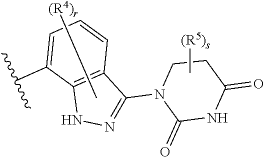

wherein each R⁴, R⁵, r and s is as defined in any of the embodiments described herein. In an embodiment, LBM is

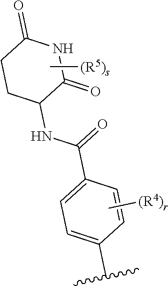

wherein each R⁴, R⁵, r and s is as defined in any of the embodiments described herein. In an embodiment, LBM is

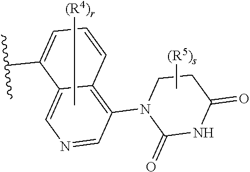

wherein each R⁴, R⁵, r and s is as defined in any of the embodiments described herein. In an embodiment, LBM is

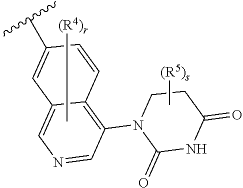

wherein each R⁴, R⁵, r and s is as defined in any of the embodiments described herein. In an embodiment, LBM is

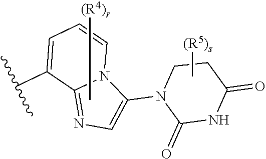

wherein each R⁴, R⁵, r and s is as defined in any of the embodiments described herein. In an embodiment, LBM is

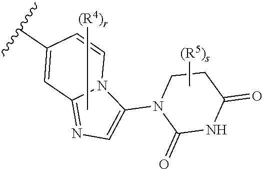

wherein each R⁴, R⁵, r and s is as defined in any of the embodiments described herein.

In an embodiment, LBM is selected from the group consisting of:

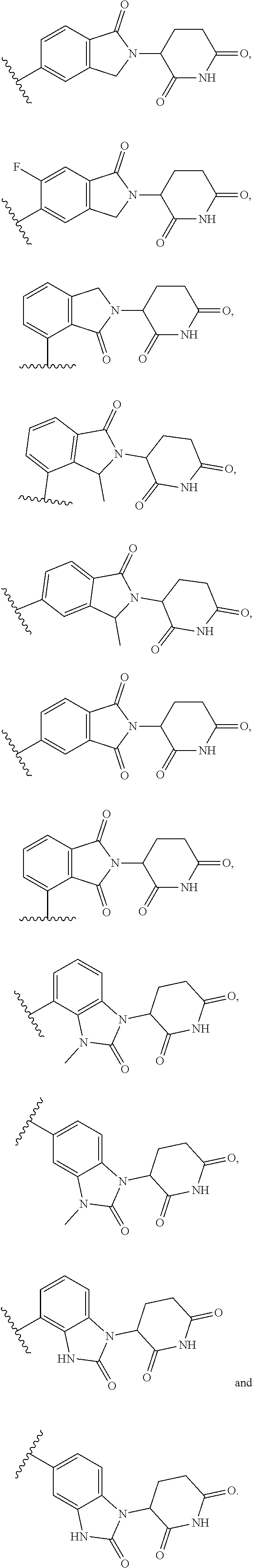

In an embodiment, LBM is selected from the group consisting of:

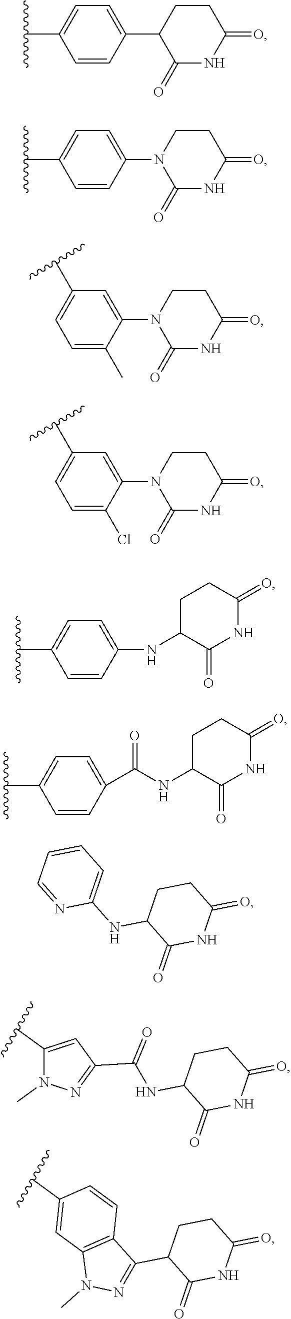

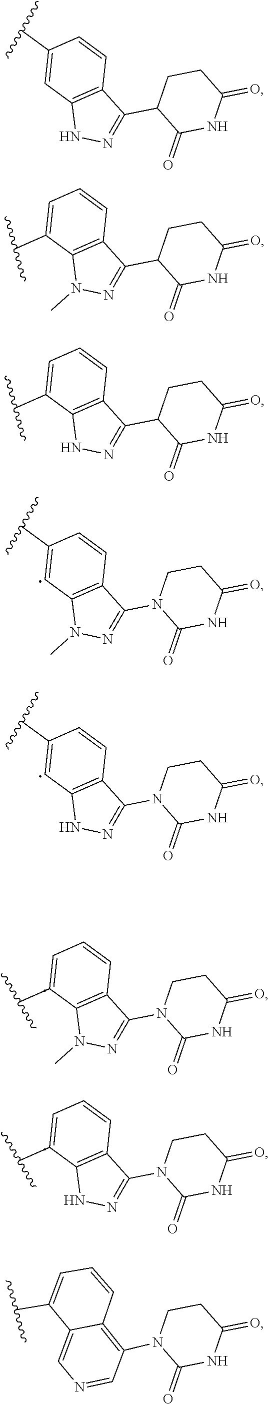

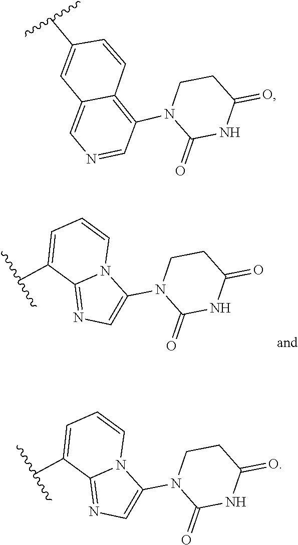 [SMILES: *c1ccc2c(N3CCC(=O)NC3=O)cncc2c1 ; *c1cccn2c(N3CCC(=O)NC3=O)cnc12 ; *c1cccn2c(N3CCC(=O)NC3=O)cnc12]

In an embodiment, LBM is selected from the group consisting of:

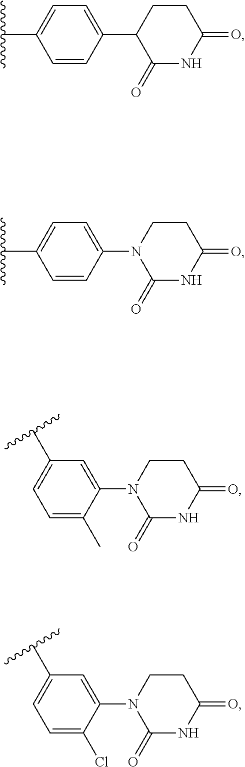 [SMILES: *c1ccc(C2CCC(=O)NC2=O)cc1 ; *c1ccc(N2CCC(=O)NC2=O)cc1 ; *c1ccc(C)c(N2CCC(=O)NC2=O)c1 ; *c1ccc(Cl)c(N2CCC(=O)NC2=O)c1]

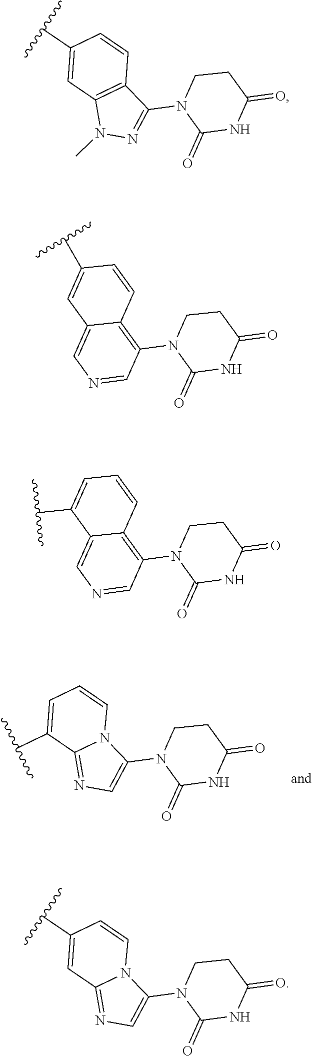 [SMILES: *c1ccc2c(N3CCC(=O)NC3=O)nn(C)c2c1 ; *c1ccc2c(N3CCC(=O)NC3=O)cncc2c1 ; *c1cccc2c(N3CCC(=O)NC3=O)cncc12 ; *c1cccn2c(N3CCC(=O)NC3=O)cnc12 ; *c1ccn2c(N3CCC(=O)NC3=O)cnc2c1]

In an embodiment, LBM is selected from p

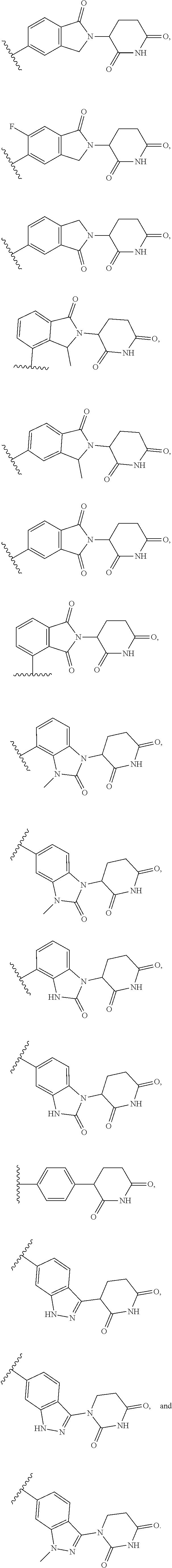

In an embodiment, LBM is selected from

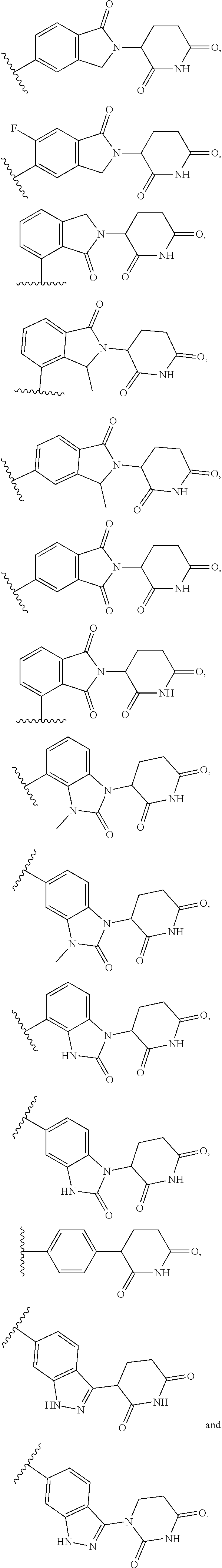

In an embodiment, LBM is selected from

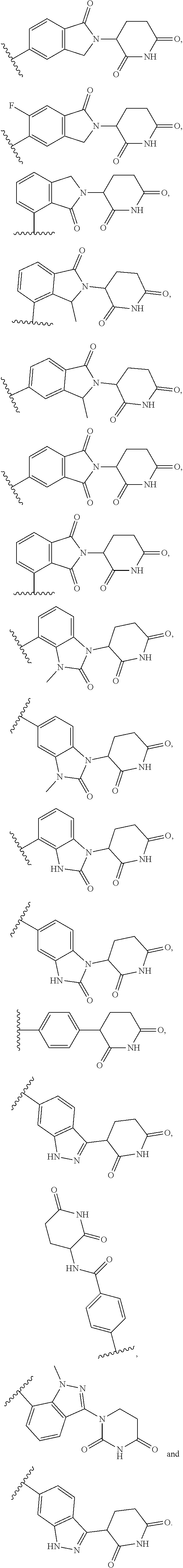

In an embodiment, LBM is selected from

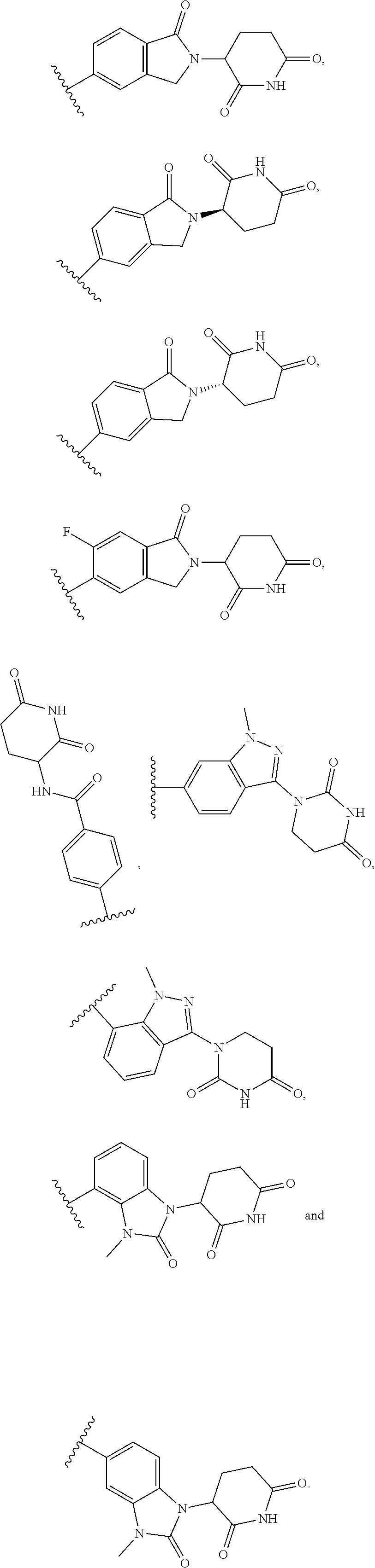

In an embodiment, LBM is selected from

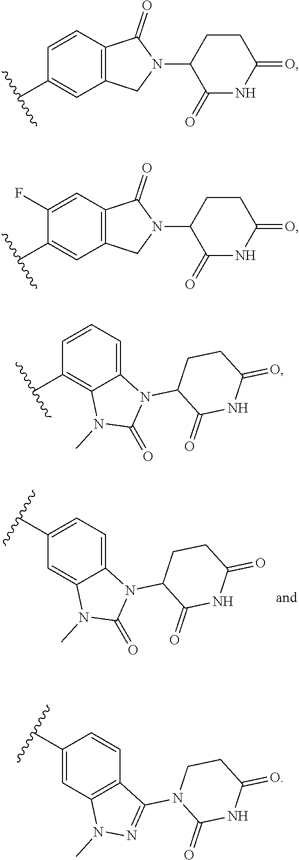 [SMILES: *c1ccc2c(c1)CN(C1CCC(=O)NC1=O)C2=O ; *c1cc2c(cc1F)C(=O)N(C1CCC(=O)NC1=O)C2 ; *c1cccc2c1n(C)c(=O)n2C1CCC(=O)NC1=O ; *c1ccc2c(c1)n(C)c(=O)n2C1CCC(=O)NC1=O ; *c1ccc2c(N3CCC(=O)NC3=O)nn(C)c2c1]

In an embodiment, LBM is

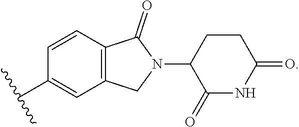 [SMILES: *c1ccc2c(c1)CN(C1CCC(=O)NC1=O)C2=O]

In an embodiment, LBM is

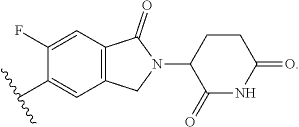 [SMILES: *c1cc2c(cc1F)C(=O)N(C1CCC(=O)NC1=O)C2]

In an embodiment, LBM is

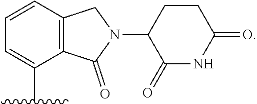 [SMILES: *c1cccc2c1C(=O)N(C1CCC(=O)NC1=O)C2]

In an embodiment, LBM is

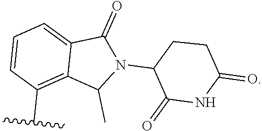 [SMILES: *c1cccc2c1C(C)N(C1CCC(=O)NC1=O)C2=O]

In an embodiment, LBM is

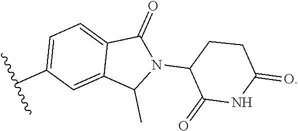 [SMILES: *c1ccc2c(c1)C(C)N(C1CCC(=O)NC1=O)C2=O]

In an embodiment, LBM is

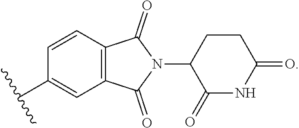 [SMILES: *c1ccc2c(c1)C(=O)N(C1CCC(=O)NC1=O)C2=O]

In an embodiment, LBM is

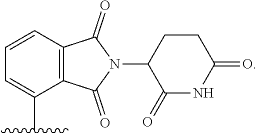 [SMILES: *c1cccc2c1C(=O)N(C1CCC(=O)NC1=O)C2=O]

In an embodiment, LBM is

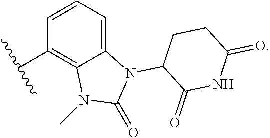 [SMILES: *c1cccc2c1n(C)c(=O)n2C1CCC(=O)NC1=O]

In an embodiment, LBM is

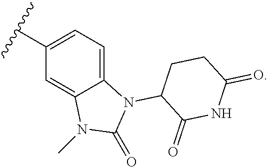 [SMILES: *c1ccc2c(c1)n(C)c(=O)n2C1CCC(=O)NC1=O]

In an embodiment, LBM is

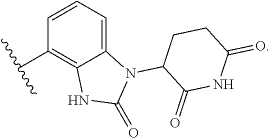 [SMILES: *c1cccc2c1[nH]c(=O)n2C1CCC(=O)NC1=O]

In an embodiment, LBM is

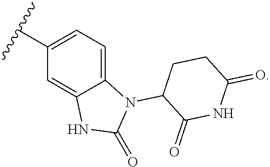 [SMILES: *c1ccc2c(c1)[nH]c(=O)n2C1CCC(=O)NC1=O]

In an embodiment, LBM is

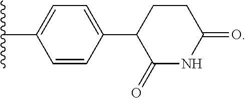 [SMILES: *c1ccc(C2CCC(=O)NC2=O)cc1]

In an embodiment, LBM is

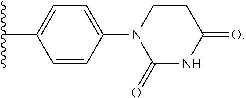 [SMILES: *c1ccc(N2CCC(=O)NC2=O)cc1]

In an embodiment, LBM is

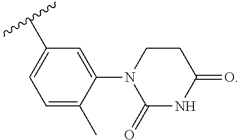 [SMILES: *c1ccc(C)c(N2CCC(=O)NC2=O)c1]

In an embodiment, LBM is

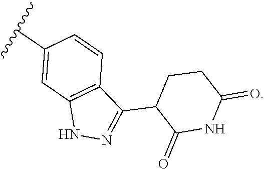 [SMILES: *c1ccc2c(C3CCC(=O)NC3=O)n[nH]c2c1]

In an embodiment, LBM is

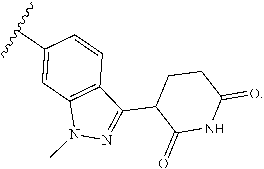 [SMILES: *c1ccc2c(C3CCC(=O)NC3=O)nn(C)c2c1]

In an embodiment, LBM is

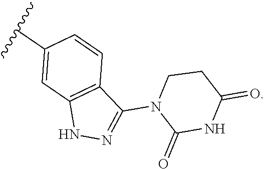 [SMILES: *c1ccc2c(N3CCC(=O)NC3=O)n[nH]c2c1]

In an embodiment, LBM is

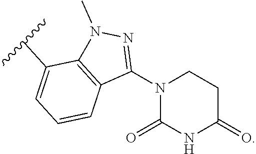 [SMILES: *c1cccc2c(N3CCC(=O)NC3=O)nn(C)c12]

In an embodiment, LBM is

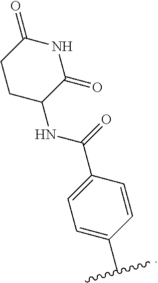 [SMILES: *c1ccc(C(=O)NC2CCC(=O)NC2=O)cc1]

In an embodiment, LBM is

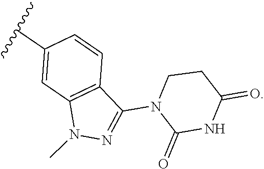 [SMILES: *c1ccc2c(N3CCC(=O)NC3=O)nn(C)c2c1]

In an embodiment, LBM is

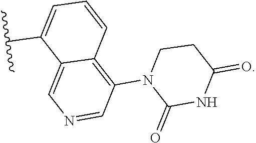 [SMILES: *c1cccc2c(N3CCC(=O)NC3=O)cncc12]

In an embodiment, LBM is

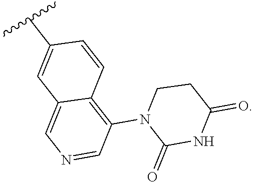 [SMILES: *c1ccc2c(N3CCC(=O)NC3=O)cncc2c1]

In an embodiment, LBM is

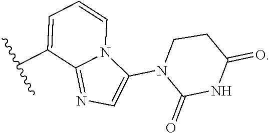 [SMILES: *c1cccn2c(N3CCC(=O)NC3=O)cnc12]

In an embodiment, LBM is

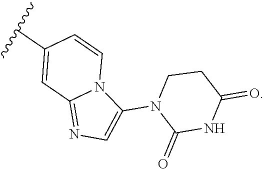 [SMILES: *c1ccn2c(N3CCC(=O)NC3=O)cnc2c1]

In an embodiment, the moiety represented by

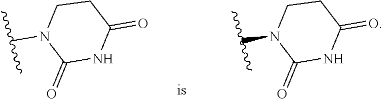

In an embodiment, the moiety represented by

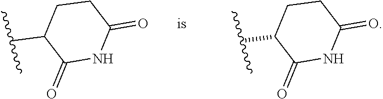

In an embodiment, LBM is

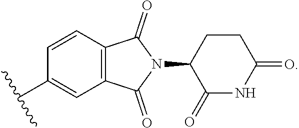 [SMILES: *c1ccc2c(c1)C(=O)N([C@H]1CCC(=O)NC1=O)C2=O]

In an embodiment, LBM is

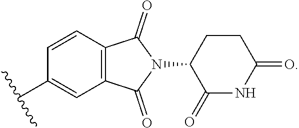 [SMILES: *c1ccc2c(c1)C(=O)N([C@@H]1CCC(=O)NC1=O)C2=O]

In an embodiment, LBM is

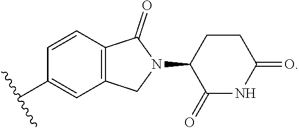 [SMILES: *c1ccc2c(c1)CN([C@H]1CCC(=O)NC1=O)C2=O]

In an embodiment, LBM is

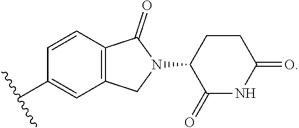 [SMILES: *c1ccc2c(c1)CN([C@@H]1CCC(=O)NC1=O)C2=O]

In an embodiment, LBM is

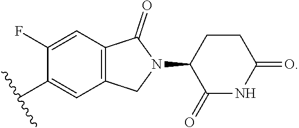 [SMILES: *c1cc2c(cc1F)C(=O)N([C@H]1CCC(=O)NC1=O)C2]

In an embodiment, LBM is

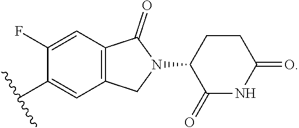 [SMILES: *c1cc2c(cc1F)C(=O)N([C@@H]1CCC(=O)NC1=O)C2]

In an embodiment, LBM is

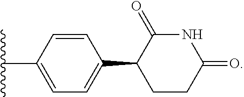 [SMILES: *c1ccc([C@@H]2CCC(=O)NC2=O)cc1]

In an embodiment, LBM is

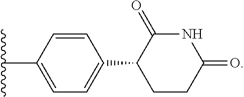 [SMILES: *c1ccc([C@H]2CCC(=O)NC2=O)cc1]

In an embodiment, LBM is

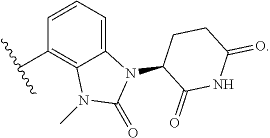 [SMILES: *c1cccc2c1n(C)c(=O)n2[C@H]1CCC(=O)NC1=O]

In an embodiment, LBM is

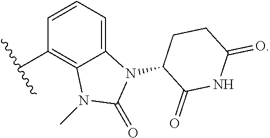 [SMILES: *c1cccc2c1n(C)c(=O)n2[C@@H]1CCC(=O)NC1=O]

In an embodiment, LBM is

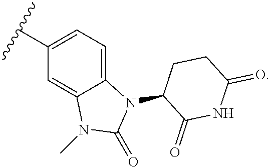 [SMILES: *c1ccc2c(c1)n(C)c(=O)n2[C@H]1CCC(=O)NC1=O]

In an embodiment, LBM is

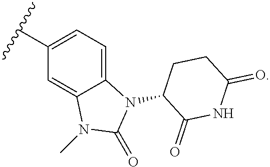 [SMILES: *c1ccc2c(c1)n(C)c(=O)n2[C@@H]1CCC(=O)NC1=O]

In an embodiment, LBM is

As generally defined herein, each instance of R^{A} is independently selected from -D, halo, CN, C₁₋₄ alkyl, C₁₋₄haloalkyl, C₃₋₆ cycloalkyl, 4-6 membered heterocyclyl, OR^{a1} SR^{a1}, SF₅, NR^{a1}R^{a1}, C₃₋₆ cycloalkyl-C₁₋₃ alkyl-, and 4-6 membered heterocyclyl-C₁₋₃ alkyl-, wherein said C₁₋₄ alkyl, C₁₋₄ haloalkyl-, C₃₋₆ cycloalkyl, 4-6 membered heterocyclyl-, C₃₋₆ cycloalkyl-C₁₋₃ alkyl-, and 4-6 membered heterocyclyl-C₁₋₃ alkyl are substituted with 0, 1, 2, 3, or 4 substituents independently selected from —D, halo, —OH, —C₁₋₄ alkyl and —OC₁₋₄ alkyl;
or, two R^{A} groups on adjacent atoms of Ring A, together with the ring atoms to which they are attached, form Ring D, wherein Ring D is selected from C₃₋₆ cycloalkyl, 4-6 membered heterocyclyl, phenyl, and 5-6 membered heteroaryl, each of which is substituted with 0, 1, 2, 3, or 4 substituents independently selected from —D, halo, —OH, —C₁₋₄ alkyl and —OC₁₋₄ alkyl, wherein R^{a1} is as defined in any of the embodiments described herein.

In an embodiment, each R^{1A} is independently selected from —D, halogen, —C₁₋₆ alkyl, —OH and —OC₁₋₄ alkyl, wherein each —C₁₋₆ alkyl is substituted with 0, 1, 2 or 3 groups independently selected from —D, —F, —OH and —OC₁₋₄ alkyl, or two R^{A} groups on adjacent atoms of Ring A, together with the ring atoms to which they are attached, form Ring D, wherein Ring D is selected from C₃₋₆ cycloalkyl, 4-6 membered heterocyclyl, phenyl, and 5-6 membered heteroaryl, each of which is substituted with 0, 1, 2, 3, or 4 substituents independently selected from —D, halo, —OH, —C₁₋₄ alkyl and —OC₁₋₄ alkyl.

In an embodiment, each R^{1A} is independently selected from —D, halogen, —C₁₋₆ alkyl, —OH and —OC₁₋₄ alkyl, wherein each —C₁₋₆ alkyl is substituted with 0, 1, 2 or 3 groups independently selected from —D, —F, —OH and —OC₁₋₄ alkyl.

In an embodiment, two R^{A} are taken together with the atoms to which they are attached to form an aryl or heteroaryl.

In an embodiment each R^{A} is independently selected from —F and —Me or two R^{A} are taken together with the atoms to which they are attached to form an aryl or heteroaryl.

In an embodiment, each R^{A} is independently selected from —F and —Me or two R^{A} are taken together with the atoms to which they are attached to form a pyridinyl or furanyl.

In an embodiment, each R^{A} is independently selected from —D, —Me, —Et, —iPr, —CF₃, —CHF₂, —F, —Cl, —OH, —OMe, OCF₃ and —OCHF₂.

In an embodiment, each R^{A} is independently selected from —D, —F, —Cl, —Me and —OH.

In an embodiment, each R^{A} is independently selected from —D, —F and —Me.

In an embodiment, each R^{A} is independently selected from —F and —Me.

In an embodiment, each R^{1A} is independently —D. In an embodiment, each R^{1A} is independently —F. In an embodiment, each R^{A} is independently —Cl— In an embodiment, each R^{A} is independently —Me. In an embodiment, each R^{A} is independently —OH.

In an embodiment, the moiety represented by

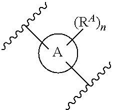

is selected from

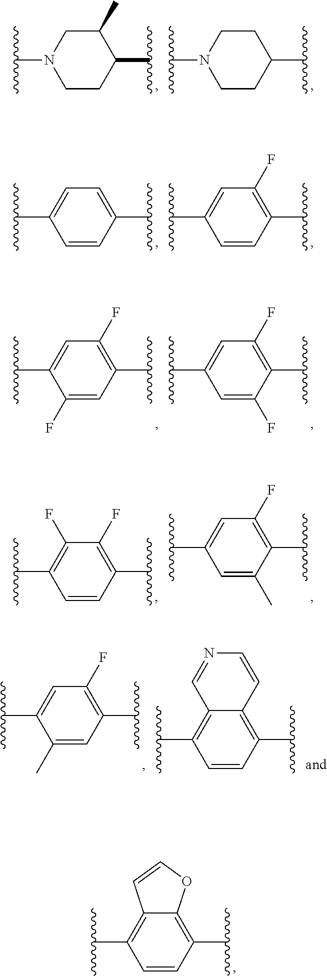

wherein the left attachment point connects to the —X— group of Formula A-I or the —S(O)₂— group of formula I and the right attachment point connects to the —NH— group of Formula A-I and formula I.

In an embodiment, the moiety represented by

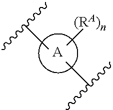

is selected from

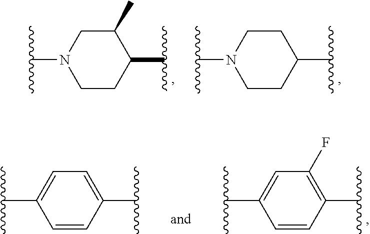

wherein the left attachment point connects to the —X— group of Formula A-I or the —S(O)₂— group of formula I and the right attachment point connects to the —NH— group of Formula A-I and formula I.

In an embodiment, the moiety represented by

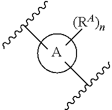

is selected from

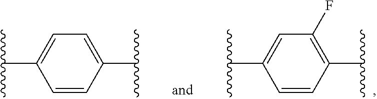 [SMILES: *c1ccc(*)cc1 ; *c1ccc(*)c(F)c1]

wherein the left attachment point connects to the —X— group of Formula A-I or the —S(O)₂— group of formula I and the right attachment point connects to the —NH— group of Formula A-I and formula I.

In an embodiment, the moiety represented by

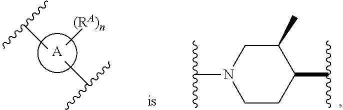

wherein the left attachment point connects to the —X— group of Formula A-I or the —S(O)₂— group of formula I and the right attachment point connects to the —NH— group of Formula A-I and formula I. In an embodiment, the moiety represented by

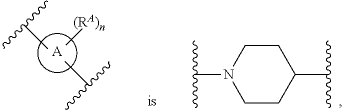

wherein the left attachment point connects to the —X— group of Formula A-I or the —S(O)₂— group of formula I and the right attachment point connects to the —NH— group of Formula A-I and formula I. In an embodiment, the moiety represented by

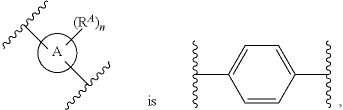

wherein the left attachment point connects to the —X— group of Formula A-I or the —S(O)₂— group of formula I and the right attachment point connects to the —NH— group of Formula A-I and formula I. In an embodiment, the moiety represented by

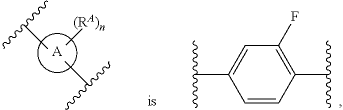

wherein the left attachment point connects to the —X— group of Formula A-I or the —S(O)₂— group of formula I and the right attachment point connects to the —NH— group of Formula A-I and formula I. In an embodiment, the moiety represented by

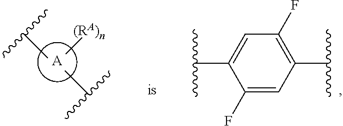

wherein the left attachment point connects to the —X— group of Formula A-I or the —S(O)₂— group of formula I and the right attachment point connects to the —NH— group of Formula A-I and formula I. In an embodiment, the moiety represented by

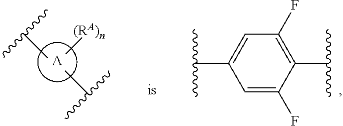

wherein the left attachment point connects to the —X— group of Formula A-I or the —S(O)₂— group of formula I and the right attachment point connects to the —NH— group of Formula A-I and formula I. In an embodiment, the moiety represented by

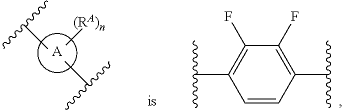

wherein the left attachment point connects to the —X— group of Formula A-I or the —S(O)₂— group of formula I and the right attachment point connects to the —NH— group of Formula A-I and formula I. In an embodiment, the moiety represented by

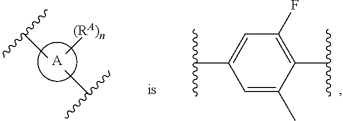

wherein the left attachment point connects to the —X— group of Formula A-I or the —S(O)₂— group of formula I and the right attachment point connects to the —NH— group of Formula A-I and formula I. In an embodiment, the moiety represented by

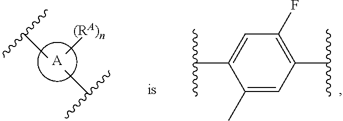

wherein the left attachment point connects to the —X— group of Formula A-I or the —S(O)₂— group of formula I and the right attachment point connects to the —NH— group of Formula A-I and formula I. In an embodiment, the moiety represented by

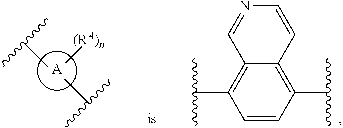

wherein the left attachment point connects to the —X— group of Formula A-I or the —S(O)₂— group of formula I and the right attachment point connects to the —NH— group of Formula A-I and formula I. In an embodiment, the moiety represented by

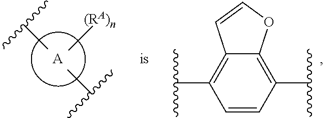

wherein the left attachment point connects to the —X— group of Formula A-I or the —S(O)₂— group of formula I and the right attachment point connects to the —NH— group of Formula A-I and formula I.

As generally defined herein, each R¹ is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocyclyl, 5-10 membered heteroaryl, C₃₋₁₀ cycloalkyl-C₁₋₄ alkyl-, 6-10 membered aryl-C₁₋₄ alkyl-, 4-10 membered heterocyclyl-C₁₋₄ alkyl-, and 5-10 membered heteroaryl-C₁₋₄ alkyl-, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocyclyl, 5-10 membered heteroaryl, C₃-10 cycloalkyl-C₁₋₄ alkyl, 6-10 membered aryl-C₁₋₄ alkyl, 4-10 membered heterocyclyl-C₁₋₄ alkyl, and 5-10 membered heteroaryl-C₁₋₄ alkyl are each substituted with 0, 1, 2, 3, or 4 independently selected R² substituents; or two R^{a1} groups attached to the same nitrogen atom together with the nitrogen to which they are attached form a 4-7-membered heterocyclyl group substituted with 0, 1, 2, 3, or 4 substituents independently selected from —D, halo, —OH, —C₁₋₄ alkyl and —OC₁₋₄ alkyl, wherein each R² is as defined in any of the embodiments described herein.

In an embodiment, R^{a1} is selected from H, C₁₋₆ alkyl, C₁₋₆ haloalkyl, C₃₋₇ cycloalkyl, phenyl, 4-7 membered heterocyclyl, 5-6 membered heteroaryl, C₃₋₇ cycloalkyl-C₁₋₄ alkyl-, phenyl-C₁₋₄ alkyl-, 4-7 membered heterocyclyl-C₁₋₄ alkyl-, and 5-6 membered heteroaryl-C₁₋₄ alkyl-, wherein said C₁₋₆ alkyl, C₁₋₆ haloalkyl, C₃₋₇ cycloalkyl, phenyl, 4-7 membered heterocyclyl, 5-6 membered heteroaryl, C₃₋₇ cycloalkyl-C₁₋₄ alkyl-, phenyl-C₁₋₄ alkyl-, 4-7 membered heterocyclyl-C₁₋₄ alkyl-, and 5-6 membered heteroaryl-C₁₋₄ alkyl are each substituted with 0, 1, 2, 3, or 4 independently selected R² substituents, wherein each R² is as defined in any of the embodiments described herein.

In an embodiment, R^{a1} is selected from H, C₁₋₆ alkyl, C₁₋₆ haloalkyl, C₃₋₆ cycloalkyl, phenyl, 4-6 membered heterocyclyl, C₃₋₆ cycloalkyl-C₁₋₃ alkyl-, and 4-6 membered heterocyclyl-C₁₋₃ alkyl-, wherein said C₁₋₆ alkyl, C₁₋₆ haloalkyl, C₃₋₆ cycloalkyl, phenyl, 4-6 membered heterocyclyl, C₃₋₆ cycloalkyl-C₁₋₃ alkyl-, and 4-6 membered heterocyclyl-C₁₋₃ alkyl- are each substituted with 0, 1, 2, or 3 independently selected R² substituents, wherein each R² is as defined in any of the embodiments described herein.

In an embodiment, R^{a1} is selected from H, C₁₋₆ alkyl, C₁₋₆ haloalkyl, C₃₋₇ cycloalkyl, phenyl, 4-7 membered heterocyclyl, and C₃₋₇ cycloalkyl-C₁₋₄ alkyl, wherein said C₁₋₆ alkyl, C₁₋₆ haloalkyl, C₃₋₇ cycloalkyl, phenyl, 4-7 membered heterocyclyl, and C₃₋₇ cycloalkyl-C₁₋₄ alkyl are each substituted with 0, 1, 2 or 3 independently selected R² substituents, wherein each R² is as defined in any of the embodiments described herein.

In an embodiment, R^{a1} is selected from H, C₁₋₆ alkyl, C₁₋₆ haloalkyl, C₃₋₆ cycloalkyl, 4-6 membered heterocyclyl, C₃₋₆ cycloalkyl-C₁₋₃ alkyl-, and 4-6 membered heterocyclyl-C₁₋₃ alkyl-, wherein said C₁₋₆ alkyl, C₁₋₆ haloalkyl, C₃₋₆ cycloalkyl, 4-6 membered heterocyclyl, C₃₋₆ cycloalkyl-C₁₋₃ alkyl-, and 4-6 membered heterocyclyl-C₁₋₃ alkyl- are each substituted with 0, 1 or 2 independently selected R² substituents, wherein each R² is as defined in any of the embodiments described herein.

In an embodiment, R^{a1} is selected from C₁₋₆ alkyl, C₁₋₆ haloalkyl and C₃₋₆ cycloalkyl, wherein said C₁₋₆ alkyl, C₁₋₆ haloalkyl and C₃₋₆ cycloalkyl are each substituted with 0, 1 or 2 independently selected R² substituents, wherein each R² is as defined in any of the embodiments described herein.

In an embodiment, R^{a1} is selected from C₁₋₆ alkyl, C₁₋₆ haloalkyl and C₃₋₆ cycloalkyl, wherein said C₁₋₆ alkyl, C₁₋₆ haloalkyl and C₃₋₆ cycloalkyl are each substituted with 0, 1 or 2 independently selected R² substituents, wherein each R² is as defined in any of the embodiments described herein.

In an embodiment, R^{a1} is selected from C₁₋₄ alkyl, C₁₋₃ fluoroalkyl and C₃₋₆ cycloalkyl, wherein said C₁₋₄ alkyl, C₁₋₃ fluoroalkyl and C₃₋₆ cycloalkyl are each substituted with 0, 1 or 2 independently selected R² substituents, wherein each R² is as defined in any of the embodiments described herein.

In an embodiment, R^{a1} is selected from —Me, —Et, —Pr, —′Pr, iso-butyl, sec-butyl, butyl, cyclopropyl, cyclobutyl, cyclopentyl, —CF₃, —CHF₂, CH₂CF₃, cyclopropyl, cyclobutyl, cyclopentyl and cyclohexyl, each substituted with 0, 1 or 2 independently selected R² substituents, wherein each R² is as defined in any of the embodiments described herein.

In an embodiment, R^{a1} is selected from ethyl, propyl, isopropyl, butyl, isobutyl, pentyl, —CH₂CF₃, —CH₂CHF₂, —CH₂CH₂CF₃, —CH₂CF₂CHF₂, —CH₂CH₂OCH₃, —CD₂CD₃, —CD(CH₃)₂, (CD₃)₂—CH—, (CD₃)₂—CD-, cyclopropyl, cyclobutyl, 3-methylcyclobutyl, 3-difluoromethylcyclobutyl, 3,3-difluorocyclobutyl, cyclopentyl, 3,3-difluorocyclopentyl, 4,4-difluorocyclohexyl, tetrahydro-1H-pyran-4-yl, tetrahydro-2H-pyran-4-yl, 2-methyltetrahydro-2H-pyran-4-yl, 3-methyltetrahydro-2H-pyran-4-yl, 2-(trifluoromethyl)tetrahydro-2H-pyran-4-yl, tetrahydrofuran-3-yl, MeO—C(O)-piperidin-4-yl, cyclopropyl-CH₂—, cyclobutyl-CH₂—, 1-trifluoromethylcyclobutyl-CH₂—, cyclopentyl-CH₂—and (tetrahydrofuran-3-yl)—CH₂—.

In an embodiment, R^{a1} is selected from ethyl, isopropyl, isobutyl, tetrahydro-1H-pyran-4-yl, cyclopropyl-CH₂—, (tetrahydrofuran-3-yl)—CH₂—, —CH₂CH₂OCH₃, —CH₂CF₃ and —CH₂CHF₂.

In an embodiment, R^{a1} is selected from ethyl, isopropyl, isobutyl, cyclobutyl, —CH₂CF₃ and —CH₂CHF₂.

In an embodiment, R^{a1} is selected from isopropyl, cyclobutyl, —CH₂CF₃ and —CH₂CHF₂. In an embodiment, R^{a1} is C₃₋₆ cycloalkyl. In an embodiment, R^{a1} is cyclobutyl. In an embodiment, R^{a1} is C₁₋₆ fluoroalkyl. In an embodiment, R^{a1} is —CH₂CF₃. In an embodiment, R^{a1} is C₁₋₃ alkyl. In an embodiment, R^{a1} is —′Pr. In an embodiment, R^{a1} is —Me. In an embodiment, R^{a1} is H.

As generally defined herein, each R^{b1} is independently selected from C₁₋₆ alkyl, C₁₋₆ hydroxyalkyl, C₃₋₉ cycloalkyl and C₂₋₆ heteroalkyl. In an embodiment, each R^{b1} is independently selected from C₁₋₆ alkyl and C₃₋₉ cycloalkyl. In an embodiment, each R^{b1} is independently selected from C₁₋₆ alkyl and C₃₋₆ cycloalkyl. In an embodiment, each R^{b1} is independently selected from —Me, —Et, —iPr, cyclopropyl, cyclobutyl, cyclopentyl, cyclohexyl. In an embodiment, each R^{b1} is independently selected from —Me, —Et and ′Pr. In an embodiment, each R^{b1} is independently —Me.

As generally defined herein, each instance of Re is independently selected from -D, halogen, —OH, and —C₁₋₆ alkyl.

In an embodiment, each instance of Re is independently selected from —D, —F, —Cl, —Me, and —OH. In an embodiment, each instance of Re is independently selected from —D, —F, —Me, and —OH. In an embodiment, each instance of R^{C} is —D. In an embodiment, each instance of R^{C} is —F. In an embodiment, each instance of R^{C} is —Cl— In an embodiment, each instance of R^{C} is —Me. In an embodiment, each instance of R^{C} is —OH.

As generally defined herein, each instance of R⁴ is independently selected from —D, halogen, —OH, and —C₁₋₆ alkyl.

In an embodiment, each R⁴ is independently selected from —D, —Me, —Et, —F, —Cl and —OH. In an embodiment, each instance of R⁴ is independently selected from —D, —F, —Cl, —Me, and —OH. In an embodiment, each instance of R⁴ is independently selected from —D, —F, —Me, and —OH. In an embodiment, each R⁴ is independently selected from —Me and —F. In an embodiment, each instance of R⁴ is —D. In an embodiment, each instance of R⁴ is —F. In an embodiment, each instance of R⁴ is —Cl— In an embodiment, each instance of R⁴ is —Me. In an embodiment, each instance of R⁴ is —OH.

As generally defined herein, each instance of R⁵ is independently selected from —D, halogen, —OH, and —C₁₋₆ alkyl.

In an embodiment, each R⁵ is independently selected from —Me, —Et, —F, —Cl and —OH. In an embodiment, each instance of R⁵ is independently selected from —D, —F, —Cl, —Me, and —OH. In an embodiment, each instance of R⁵ is independently selected from —D, —F, -Me, and —OH. In an embodiment, each instance of R⁵ is —D. In an embodiment, each instance of R⁵ is —F. In an embodiment, each instance of R⁵ is —Cl— In an embodiment, each instance of R⁵ is —Me. In an embodiment, each instance of R⁵ is —OH.

As generally defined herein, each instance of R is independently hydrogen and —C₁₋₆ alkyl. In an embodiment, R is H. In an embodiment R is —C₁₋₆ alkyl. In an embodiment, R is selected from H and —Me. In an embodiment, R is —Me.

As generally defined herein, each L¹ is independently selected from a bond and —N(R′), wherein R′ is as defined in any of the embodiments described herein. In an embodiment, L¹ is a bond. In an embodiment, L¹ is —N(R′), wherein R′ is as defined in any of the embodiments described herein. In an embodiment, L¹ is selected from a bond, —NH— and —NMe—. In an embodiment, L¹ is selected from a bond and —NH—. In an embodiment, L¹ is selected from a bond —NMe—. In an embodiment, L¹ is —NH—. In an embodiment, L¹ is —NMe—.

As generally defined herein, each L² independently selected from a bond and —N(R′), wherein R′ is as defined in any of the embodiments described herein.

In an embodiment, L² is a bond. In an embodiment, L² is —N(R′), wherein R′ is as defined in any of the embodiments described herein. In an embodiment, L² is selected from a bond, —NH— and —NMe—. In an embodiment, L² is selected from a bond and —NH—. In an embodiment, L² is selected from a bond —NMe—. In an embodiment, L² is —NH—. In an embodiment, L² is —NMe—.

In an embodiment, L¹ is a bond and L² is a bond or N(R′)—. In an embodiment, L¹ is a bond and L² is a bond or NMe—.

In an embodiment, L¹ is a bond and L² is a bond or —NH—.

As generally defined herein, each R′ is independently selected from H and C₁₋₆ alkyl.

As generally defined herein, each R″ is independently selected from H and C₁₋₆ alkyl. In an embodiment, R″ is independently selected from H and —Me. In an embodiment, R″ is H. In an embodiment, R″ is —Me.

As generally defined herein, each R⁷ is independently selected from —C₁₋₄ alkyl and halo. In an embodiment, each R⁷ is —C₁₋₄ alkyl. In an embodiment, each R⁷ is halo. In an embodiment, each R⁷ is selected from —Me, —Et, —′Pr, —F, and —Cl. In an embodiment, each R⁷ is selected from —Me, —Et, —′Pr and —F. In an embodiment, each R⁷ is selected from —Me, —Et and —′Pr. In an embodiment, each R⁷ is —Me. In an embodiment, each R⁷ is —Et. In an embodiment, each R⁷ is —′Pr. In an embodiment, each R⁷ is selected from —Me and —F. In an embodiment, each R⁷ is —F.

As generally defined herein, n is 0, 1, 2, 3, or 4. In an embodiment, n is 0, 1, 2 or 3. In an embodiment, n is 0, 1 or 2. In an embodiment, n is 0 or 1. In an embodiment, n is 0. In an embodiment, n is 1. In an embodiment, n is 2. In an embodiment, n is 3. In an embodiment, n is 4.

As generally defined herein, r is 0, 1, 2, 3, or 4. In an embodiment, r is 0, 1, 2 or 3. In an embodiment, r is 0, 1 or 2. In an embodiment, r is 0 or 1. In an embodiment, r is 0. In an embodiment, r is 1. In an embodiment, r is 2. In an embodiment, r is 3. In an embodiment, r is 4.

As generally defined herein, s is 0, 1, 2, 3, or 4. In an embodiment, s is 0, 1, 2 or 3. In an embodiment, s is 0, 1 or 2. In an embodiment, s is 0 or 1. In an embodiment, s is 0. In an embodiment, s is 1. In an embodiment, s is 2. In an embodiment, s is 3. In an embodiment, s is 4.

As generally defined herein, q is 0, 1, 2, 3, 4, 5, 6, 7, 8, 9 or 10. In an embodiment, q is 1, 2, 3, 4, 5, 7 or 9. In an embodiment, q is 0. In an embodiment, q is 1. In an embodiment, q is 2. In an embodiment, q is 3. In an embodiment, q is 4. In an embodiment, q is 5. In an embodiment, q is 6. In an embodiment, q is 7. In an embodiment, q is 8. In an embodiment, q is 9. In an embodiment, q is 10.

As generally defined herein, each of W¹, W², W³ and W⁴ is selected from CH or N, provided that no more than two of W¹, W², W³ and W⁴ are N. In one embodiment, W¹ is N and W², W³ and W⁴ are CH. In one embodiment, W² is N and W¹, W³ and W⁴ are CH. In one embodiment, W³ is N and W¹, W² and W⁴ are CH. In one embodiment, W⁴ is N and W¹, W² and W³ are CH. In one embodiment, W¹ and W² are N and W³ and W⁴ are CH. In one embodiment, W¹ and W³ are N and W² and W⁴ are CH. In one embodiment, W¹ and W⁴ are N and W² and W³ are CH. In one embodiment, W² and W³ are N and W¹ and W⁴ are CH. In one embodiment, W² and W⁴ are N and W¹ and W³ are CH. In one embodiment, W³ and W⁴ are N and W¹ and W³ are CH.

In an embodiment, the compound is of Formula A-I-A:

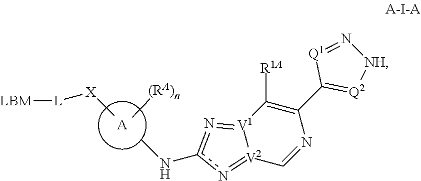

V², Q¹, Q², L, LBM and n are as defined in any of the embodiments described herein.

In an embodiment, the compound is of Formula A-I-B:

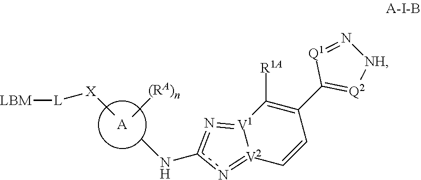

wherein Ring A, R^{1A}, R^{A}, X, V¹, V², Q¹, Q², L, LBM and n are as defined in any of the embodiments described herein.

In an embodiment, the compound is of Formula A-I-1:

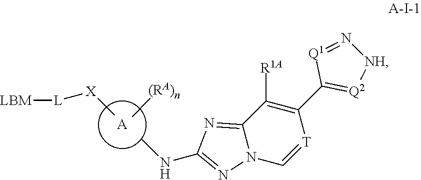

wherein Ring A, R^{1A}, R^{A}, X, Q¹, Q², T, L, LBM and n are as defined in any of the embodiments described herein.

In an embodiment, the compound is of Formula A-I-2:

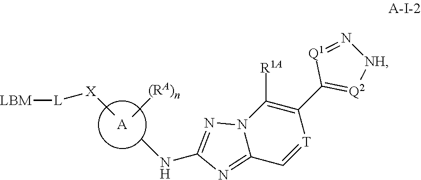

wherein Ring A, R^{1A}, R^{A}, X, Q¹ Q², T, L, LBM and n are as defined in any of the embodiments described herein.

In an embodiment, the compound is of Formula A-I—Al:

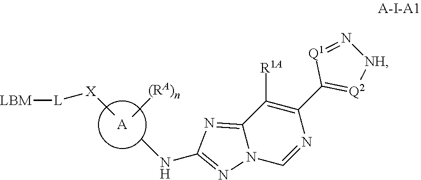

wherein Ring A, R^{1A}, R^{A}, X, Q¹, Q², L, LBM and n are as defined in any of the embodiments described herein.

In an embodiment, the compound is of Formula A-I-A2:

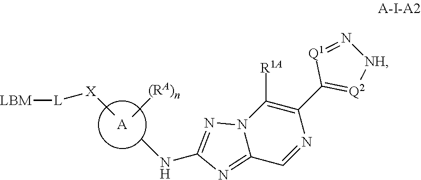

wherein Ring A, R^{1A}, R^{A}, X, Q¹, Q², L, LBM and n are as defined in any of the embodiments described herein.

In an embodiment, the compound is of Formula A-I-B1:

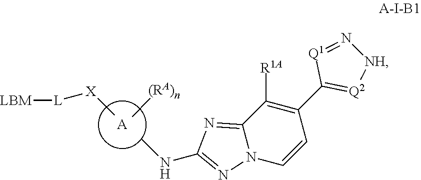

wherein Ring A, R^{1A}, R^{A}, X, Q¹, Q², L, LBM and n are as defined in any of the embodiments described herein.

In an embodiment, the compound is of Formula A-I-B2:

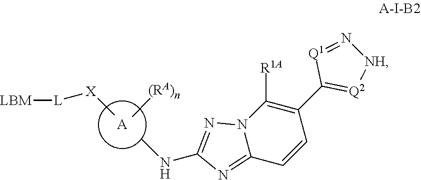

wherein Ring A, R^{1A}, R^{A}, X, V¹, V², Q¹, Q², L, LBM and n are as defined in any of the embodiments described herein.

In an embodiment, the compound is a compound of Formula A-II:

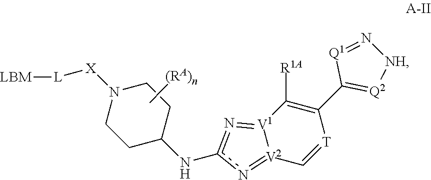

wherein R^{1A}, R^{A}, X, V¹, V², T, Q¹ Q², L, LBM and n are as defined in any of the embodiments described herein.

In an embodiment, the compound is of Formula A-II-A:

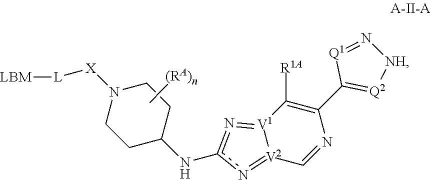

wherein R^{1A}, R^{A}, X, V¹, V², Q¹ Q², L, LBM and n are as defined in any of the embodiments described herein.

In an embodiment, the compound is of Formula A-II-B:

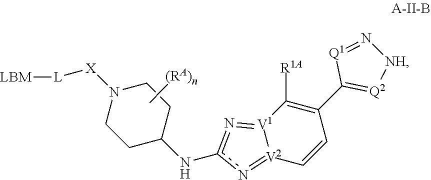

wherein R^{1A}, R^{A}, X, V¹, V², Q¹ Q², L, LBM and n are as defined in any of the embodiments described herein.

In an embodiment, the compound is of Formula A-II-1:

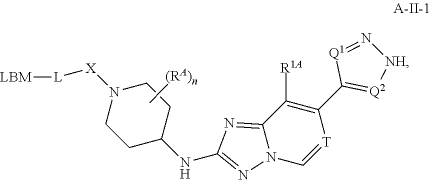

wherein R^{1A}, R^{A}, X, T, Q¹, Q², L, LBM and n are as defined in any of the embodiments described herein.

In an embodiment, the compound is of Formula A-II-2:

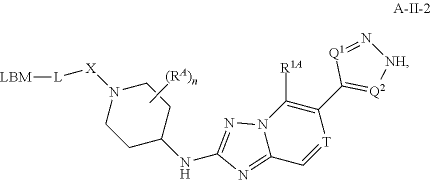

wherein R^{1A}, R^{A}, X, T, Q¹, Q², L, LBM and n are as defined in any of the embodiments described herein.

In an embodiment, the compound is of Formula A-IT-A1:

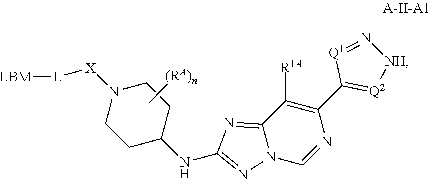

wherein R^{1A}, R^{A}, X, Q¹, Q², L, LBM and n are as defined in any of the embodiments described herein.

In an embodiment, the compound is of Formula A-II-A2:

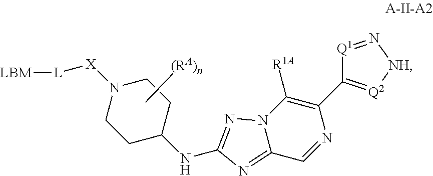

wherein R^{1A}, R^{A}, X, Q¹, Q², L, LBM and n are as defined in any of the embodiments described herein.

In an embodiment, the compound is of Formula A-II-B1:

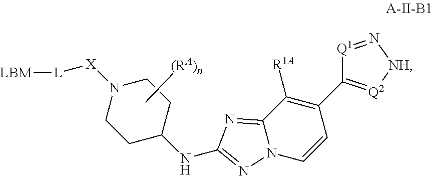

wherein R^{1A}, R^{A}, X, Q¹, Q², L, LBM and n are as defined in any of the embodiments described herein.

In an embodiment, the compound is of Formula A-II-B2:

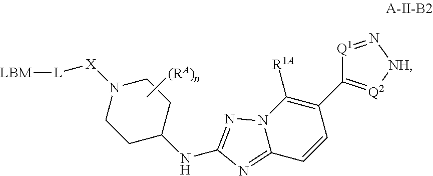

wherein R^{1A}, R^{A}, X, Q¹, Q² L, LBM and n are as defined in any of the embodiments described herein.

In an embodiment, the compound is a compound of Formula A-III:

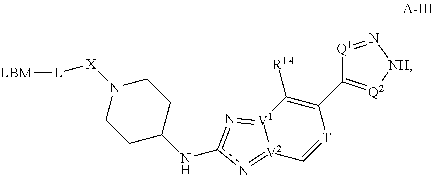

wherein R^{1A}, X, V, V², T, Q¹, Q², L and LBM are as defined in any of the embodiments described herein.

In an embodiment, the compound is of Formula A-ITT-A:

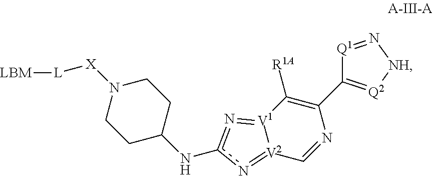

wherein R^{1A}, X, V¹, V², Q¹, Q², L and LBM are as defined in any of the embodiments described herein.

In an embodiment, the compound is of Formula A-ITT-B:

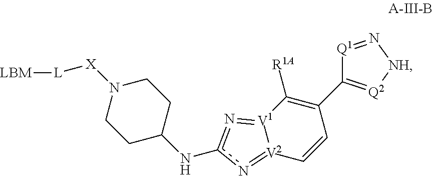

wherein R^{1A}, X, V¹, V², Q¹, Q², L and LBM are as defined in any of the embodiments described herein.

In an embodiment, the compound is of Formula A-III-1:

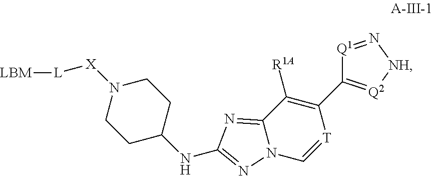

wherein R^{1A}, X, T, Q, Q², L and LBM are as defined in any of the embodiments described herein.

In an embodiment, the compound is of Formula A-III-2:

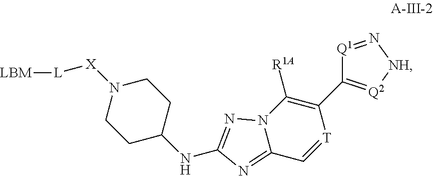

wherein R^{1A}, X, T, Q¹, Q², L and LBM are as defined in any of the embodiments described herein.

In an embodiment, the compound is of Formula A-ITT-A1:

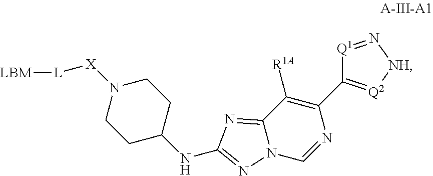

wherein R^{1A} X, Q¹, Q², L and LBM are as defined in any of the embodiments described herein.

In an embodiment, the compound is of Formula A-III-A2:

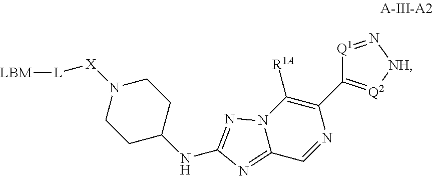

wherein R″, X, Q¹, Q², L and LBM are as defined in any of the embodiments described herein.

In an embodiment, the compound is of Formula A-ITT-B1:

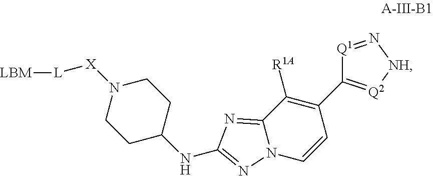

wherein R″, X, Q¹, Q², L and LBM are as defined in any of the embodiments described herein.

In an embodiment, the compound is of Formula A-III-B2:

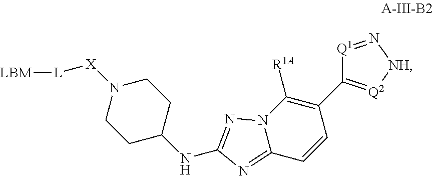

wherein R^{1A} X, Q¹, Q², L and LBM are as defined in any of the embodiments described herein.

In an embodiment, the compound is a compound of Formula A-IV:

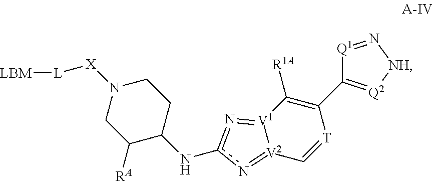

wherein R^{1A}, R^{A}, X, V¹, V², T, Q¹, Q², L and LBM are as defined in any of the embodiments described herein.

In an embodiment, the compound is of Formula A-IV-A:

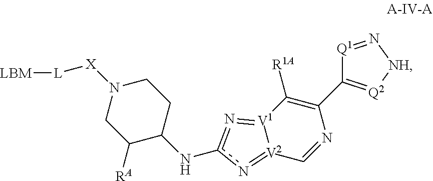

wherein R^{1A}, R^{A}, X, V¹, V², Q¹, Q², L and LBM are as defined in any of the embodiments described herein.

In an embodiment, the compound is of Formula A-IV-B:

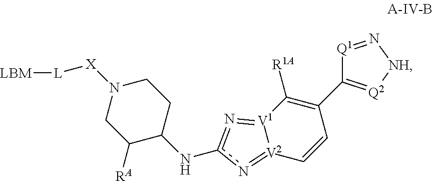

wherein R^{1A}, R^{A}, X, V¹, V², Q¹i Q², L and LBM are as defined in any of the embodiments described herein.

In an embodiment, the compound is of Formula A-IV-1:

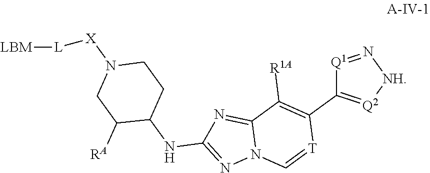

wherein R^{1A}, R^{A}, X, T, Q¹, Q², L and LBM are as defined in any of the embodiments described herein.

In an embodiment, the compound is of Formula A-IV-2:

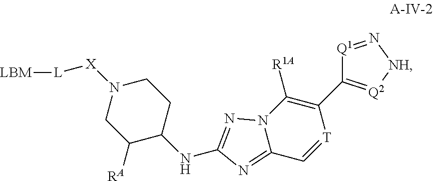

wherein R^{1A}, R^{A}, X, T, Q¹, Q², L and LBM are as defined in any of the embodiments described herein.

In an embodiment, the compound is of Formula A-IV-A1:

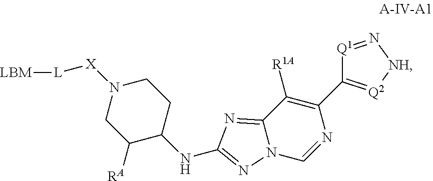

wherein R^{1A}, R^{A}, X, Q¹, Q², L and LBM are as defined in any of the embodiments described herein.

In an embodiment, the compound is of Formula A-IV-A2:

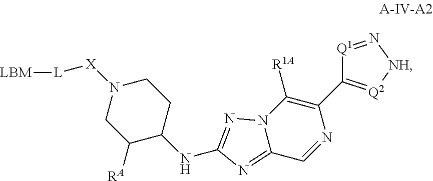

wherein R^{1A}, R^{A}, X, Q¹, Q², L and LBM are as defined in any of the embodiments described herein.

In an embodiment, the compound is of Formula A-IV-B1:

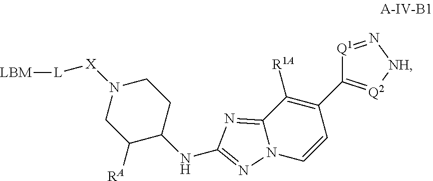

wherein R^{1A}, R^{A}, X, Q¹, Q², L and LBM are as defined in any of the embodiments described herein.

In an embodiment, the compound is of Formula A-IV-B2:

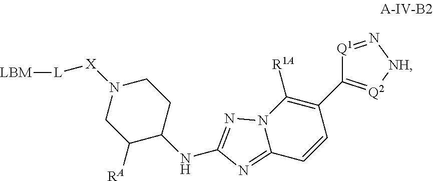

wherein R^{1A}, R^{A}, X, Q¹, Q², L and LBM are as defined in any of the embodiments described herein.

In an embodiment, the compound is a compound of Formula A-V:

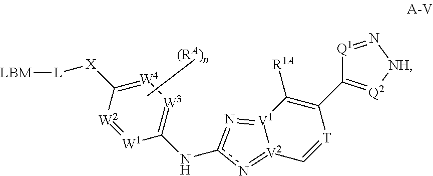

wherein W¹, W², W³, W⁴, R^{1A}, R^{A}, X, V¹, V², Q¹, Q², L, LBM and n are as defined in any of the embodiments described herein.

In an embodiment, the compound is of Formula A-V-A:

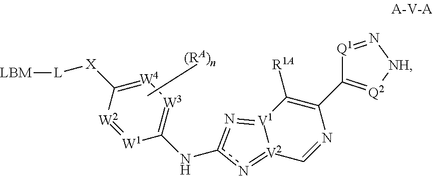

wherein W¹, W², W³, W⁴, R^{1A}, R^{A}, X, V¹, V², Q¹, Q², L, LBM and n are as defined in any of the embodiments described herein.

In an embodiment, the compound is of Formula A-V-B:

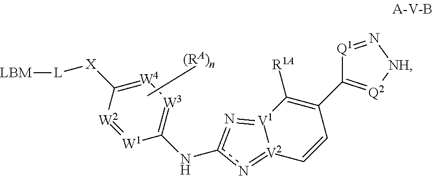

wherein W¹, W², W³, W⁴, R^{1A}, R^{A}, X, V¹, V², Q¹, Q², L, LBM and n are as defined in any of the embodiments described herein.

In an embodiment, the compound is a compound of Formula A-V-1:

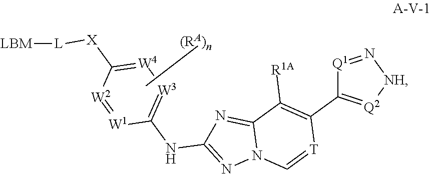

wherein W¹, W², W³, W⁴, R^{1A}, R^{A}, X, T, Q¹, Q², L, LBM and n are as defined in any of the embodiments described herein.

In an embodiment, the compound is a compound of Formula A-V-2:

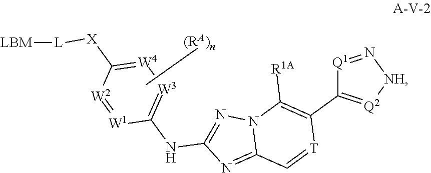

wherein W¹, W², W³, W⁴, R^{1A}, R^{A}, X, T, Q¹, Q², L, LBM and n are as defined in any of the embodiments described herein.

In an embodiment, the compound is a compound of Formula A-V-A1:

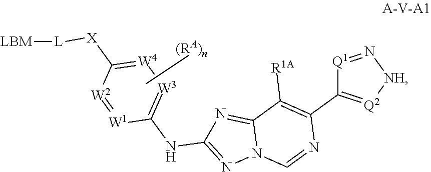

wherein W¹, W², W³, W⁴, R^{1A}, R^{A}, X, Q¹, Q², L, LBM and n are as defined in any of the embodiments described herein.

In an embodiment, the compound is a compound of Formula A-V-A2:

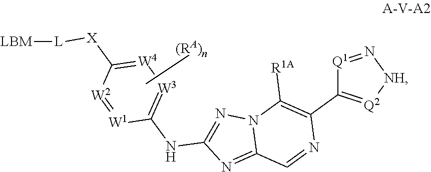

wherein W¹, W², W³, W⁴, R^{1A}, R^{A}, X, Q¹, Q², L, LBM and n are as defined in any of the embodiments described herein.

In an embodiment, the compound is a compound of Formula A-V-B1:

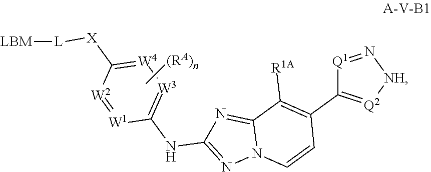

wherein W¹, W², W³, W⁴, R^{1A}, R^{A}, X, Q¹, Q², L, LBM and n are as defined in any of the embodiments described herein.

In an embodiment, the compound is a compound of Formula A-V-B2:

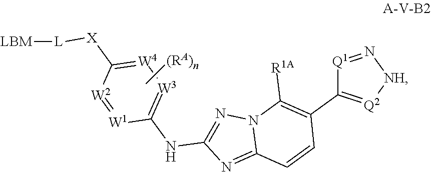

wherein W¹, W², W³, W⁴, RIA, R^{A}, X, Q¹, Q², L, LBM and n are as defined in any of the embodiments described herein.

In an embodiment, the compound is a compound of Formula A-VI:

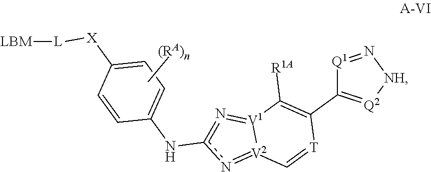

wherein R^{1A}, R^{A}, X, V¹, V², T, Q¹, Q², L, LBM and n are as defined in any of the embodiments described herein.

In an embodiment, the compound is of Formula A-VI-A:

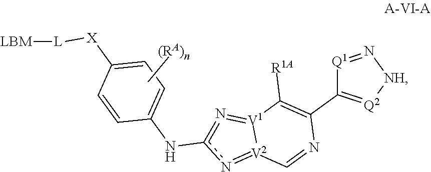

wherein R^{1A}, R^{A}, X, V¹, V², Q¹, Q², L, LBM and n are as defined in any of the embodiments described herein.

In an embodiment, the compound is of Formula A-VI-B:

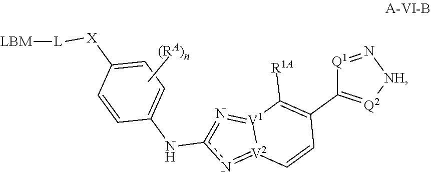

wherein R^{1A}, R^{A}, X, V¹, V², Q¹, Q², L, LBM and n are as defined in any of the embodiments described herein.

In an embodiment, the compound is a compound of Formula A-VI-1:

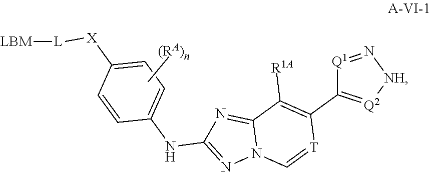

wherein R^{1A}, R^{A}, X, T, Q¹, Q², L, LBM and n are as defined in any of the embodiments described herein.

In an embodiment, the compound is a compound of Formula A-VI-2:

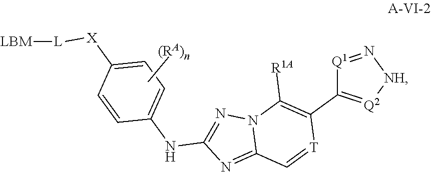

wherein R^{1A}, R^{A}, X, T, Q¹, Q², L, LBM and n are as defined in any of the embodiments described herein.

In an embodiment, the compound is a compound of Formula A-VI—Al:

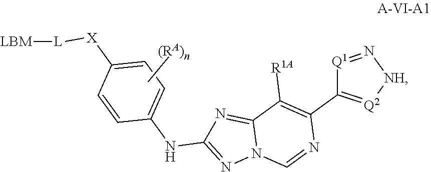

wherein R^{1A}, R^{A}, X, Q¹, Q², L, LBM and n are as defined in any of the embodiments described herein.

In an embodiment, the compound is a compound of Formula A-VI-A2:

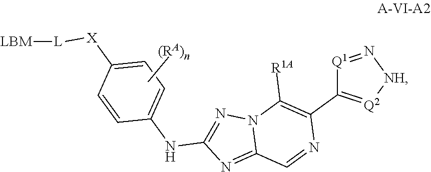

wherein R^{1A}, R^{A}, X, Q¹, Q², L, LBM and n are as defined in any of the embodiments described herein.

In an embodiment, the compound is a compound of Formula A-VI-B1:

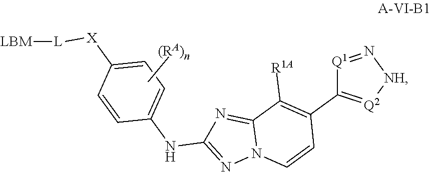

wherein R^{1A}, R^{A}, X, Q, Q², L LBM and n are as defined in any of the embodiments described herein.

In an embodiment, the compound is a compound of Formula A-VI-B2:

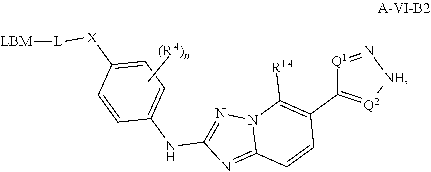

wherein R^{1A}, R^{A}, X, Q¹, Q² L, LBM and n are as defined in any of the embodiments described herein.

In an embodiment, the compound is a compound of Formula A-VII:

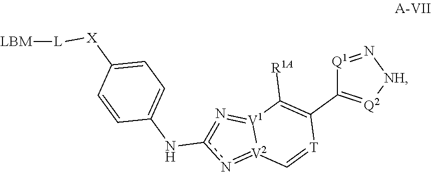

wherein R^{1A}, R^{A}, X, V¹, V², T, Q¹ Q², L, LBM and n are as defined in any of the embodiments described herein.

In an embodiment, the compound is of Formula A-VII-A:

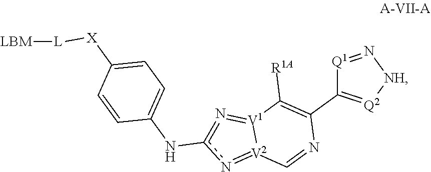

wherein R^{1A}, R^{A}, X, V¹, V², Q¹, Q², L, LBM and n are as defined in any of the embodiments described herein.

In an embodiment, the compound is of Formula A-VII-B:

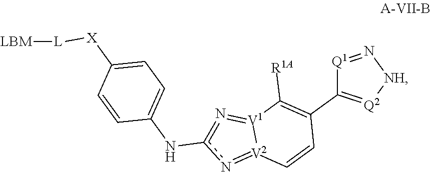

wherein R^{1A}, R^{A}, X, V¹, V² Q¹ Q², L, LBM and n are as defined in any of the embodiments described herein.

In an embodiment, the compound is a compound of Formula A-VII-1:

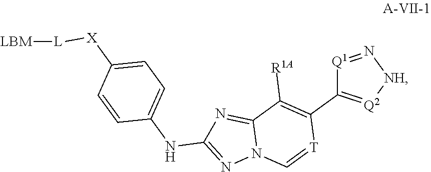

wherein R^{1A}, R^{A}, X, T, Q¹, Q², L, LBM and n are as defined in any of the embodiments described herein.

In an embodiment, the compound is a compound of Formula A-VII-2:

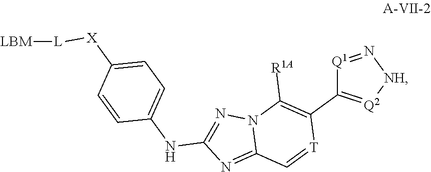

wherein R^{1A}, R^{A}, X, T, Q¹, Q², L, LBM and n are as defined in any of the embodiments described herein.

In an embodiment, the compound is a compound of Formula A-VII-A1:

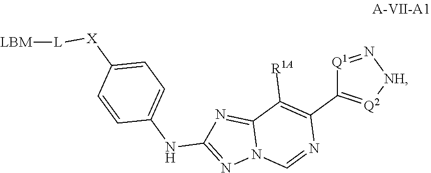

wherein R^{1A}, R^{A}, X, Q¹, Q², L, LBM and n are as defined in any of the embodiments described herein.

In an embodiment, the compound is a compound of Formula A-VII-A2:

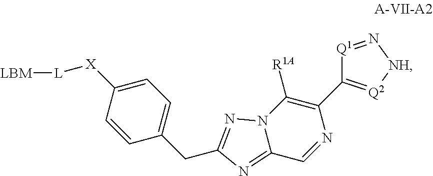

wherein R^{1A}, R^{A}, X, Q¹, Q², L, LBM and n are as defined in any of the embodiments described herein.

In an embodiment, the compound is a compound of Formula A-VII-B1:

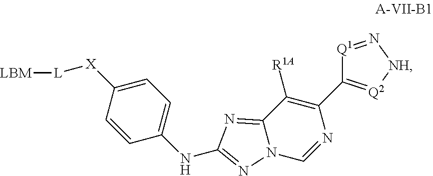

wherein R^{1A}, R^{A}, X, Q¹, Q², L, LBM and n are as defined in any of the embodiments described herein.

In an embodiment, the compound is a compound of Formula A-VII-B2:

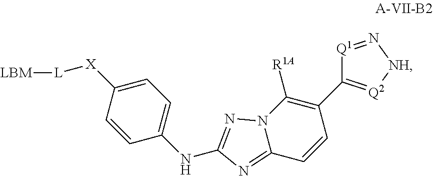

wherein R^{1A}, R^{A}, X, Q¹, Q² L, LBM and n are as defined in any of the embodiments described herein.

In an embodiment, the compound is a compound of Formula A-VIII:

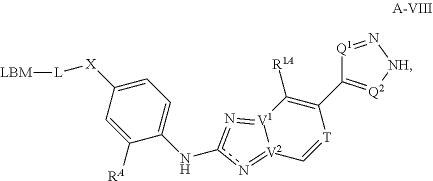

wherein R^{1A}, R^{A}, X, V¹, V², T, Q¹ Q², L and LBM are as defined in any of the embodiments described herein.

In an embodiment, the compound is of Formula A-VIII-A:

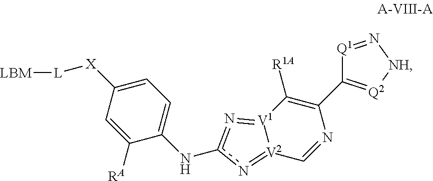

wherein R^{1A}, R^{A}, X, V¹, V², Q¹ Q², L and LBM are as defined in any of the embodiments described herein.

In an embodiment, the compound is of Formula A-VIII-B:

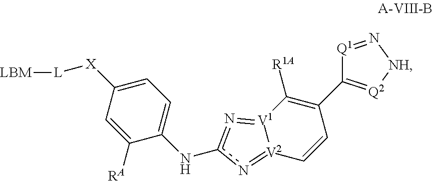

wherein R^{1A}, R^{A}, X, V¹, V², Q¹ Q², L and LBM are as defined in any of the embodiments described herein.

In an embodiment, the compound is a compound of Formula A-VIII-1:

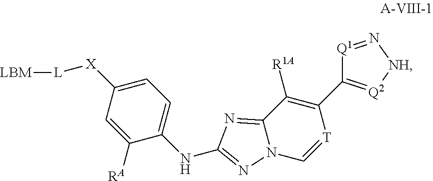

wherein R^{1A}, R^{A}, X, T, Q¹, Q², L and LBM are as defined in any of the embodiments described herein.

In an embodiment, the compound is a compound of Formula A-VIII-2:

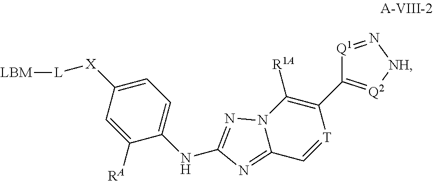

wherein R^{1A}, R^{A}, X, T, Q¹, Q², L and LBM are as defined in any of the embodiments described herein.

In an embodiment, the compound is a compound of Formula A-VIII-A1:

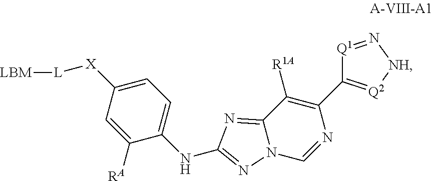

wherein R^{1A}, R^{A}, X, Q¹, Q², L and LBM are as defined in any of the embodiments described herein.

In an embodiment, the compound is a compound of Formula A-VIII-A2:

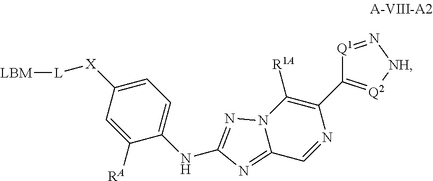

wherein R^{1A}, R^{A}, X, Q¹, Q², L and LBM are as defined in any of the embodiments described herein.

In an embodiment, the compound is a compound of Formula A-VIII-B1:

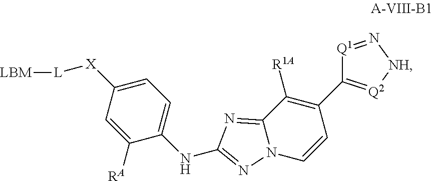

wherein R^{1A}, R^{A}, X, Q¹, Q², L and LBM are as defined in any of the embodiments described herein.

In an embodiment, the compound is a compound of Formula A-VIII-B2:

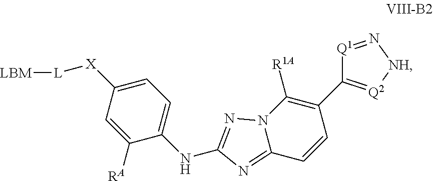

wherein R^{1A}, R^{A}, X, V¹, V², T, Q¹, Q², L and LBM are as defined in any of the embodiments described herein.

In an embodiment, provided is a compound of Formula I-1:

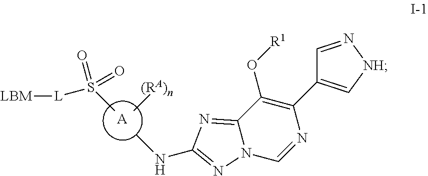

wherein each R¹, R^{A}, Ring A, L, LBM and n are as defined in any of the embodiments described herein.

In an embodiment, provided is a compound of Formula I-2:

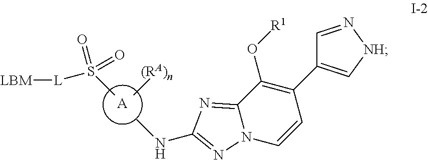

wherein each R¹, R^{A}, Ring A, L, LBM and n are as defined in any of the embodiments described herein.

In an embodiment, provided is a compound of Formula II:

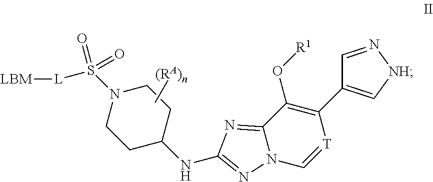

wherein each R¹, R^{A}, T, L, LBM and n are as defined in any of the embodiments described herein.

In an embodiment, provided is a compound of Formula II-1:

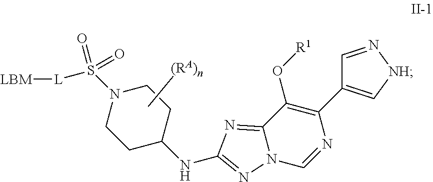

wherein each R¹, R^{A}, L, LBM and n are as defined in any of the embodiments described herein.

In an embodiment, provided is a compound of Formula II-2:

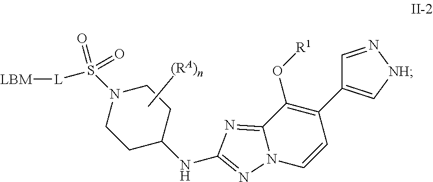

wherein each R¹, R^{A}, L, LBM and n are as defined in any of the embodiments described herein.

In an embodiment, provided is a compound of Formula II-a:

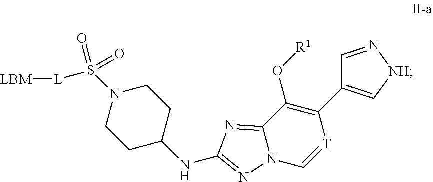

wherein each R¹, T, L and LBM are as defined in any of the embodiments described herein.

In an embodiment, provided is a compound of Formula II-a-1:

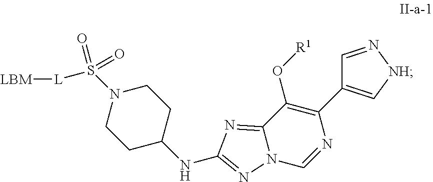

wherein each R¹, L and LBM are as defined in any of the embodiments described herein.

In an embodiment, provided is a compound of Formula II-a-2:

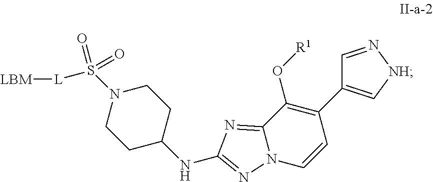

wherein each R¹, L and LBM are as defined in any of the embodiments described herein.

In an embodiment, provided is a compound of Formula II-b:

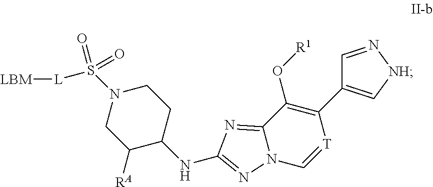

wherein each R¹, R^{A}, T, L and LBM are as defined in any of the embodiments described herein.

In an embodiment, provided is a compound of Formula II-b-1:

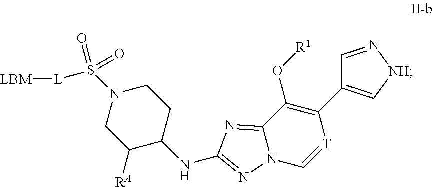

wherein each R¹, R^{A}, L and LBM are as defined in any of the embodiments described herein.

In an embodiment, provided is a compound of Formula II-b-2:

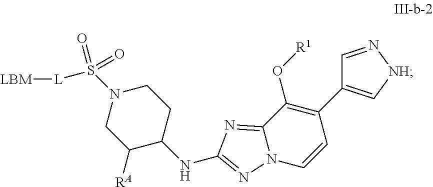

wherein each R¹, R^{A}, L and LBM are as defined in any of the embodiments described herein.

In an embodiment, R^{A} and the —NH— group attached to the piperidine ring are in a cis configuration. In an embodiment, R^{A} and the —NH— group attached to the piperidine ring are in a trans configuration. In an embodiment, R^{A} and the —NH— group attached to the piperidine ring are in an R, S configuration. In an embodiment, R^{A} and the —NH— group attached to the piperidine ring are in an R, R configuration. In an embodiment, R^{A} and the —NH— group attached to the piperidine ring are in an S, S configuration. In an embodiment, R^{A} and the —NH— group attached to the piperidine ring are in an S, R configuration.

In an embodiment, provided is a compound of Formula III:

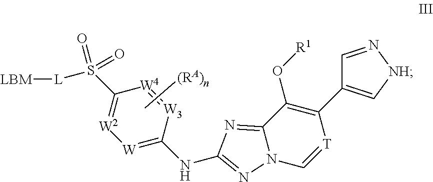

wherein each W¹, W², W³, W⁴′ R¹, R^{A}, L, T, LBM and n are as defined in any of the embodiments described herein.

In an embodiment, provided is a compound of Formula III-1:

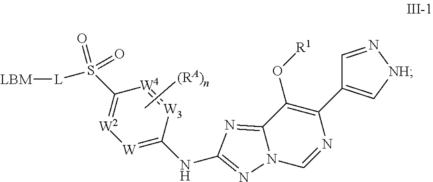

wherein each W¹, W², W³, W⁴′ R¹, R^{A}, L, LBM and n are as defined in any of the embodiments described herein.

In an embodiment, provided is a compound of Formula III-2:

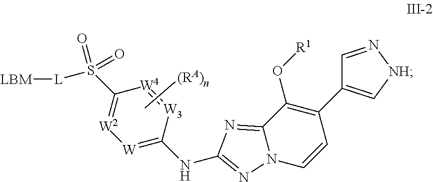

wherein each W¹, W², W³, W⁴′ R¹, R^{A}, L, LBM and n are as defined in any of the embodiments described herein.

In an embodiment, provided is a compound of Formula III-a:

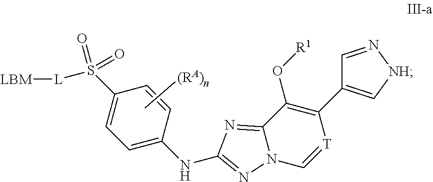

wherein each R¹, R^{A}, T, L and LBM are as defined in any of the embodiments described herein.

In an embodiment, provided is a compound of Formula III-a-1:

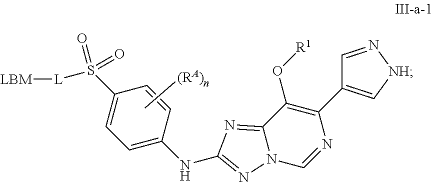

wherein each R¹, R^{A}, L and LBM are as defined in any of the embodiments described herein.

In an embodiment, provided is a compound of Formula III-a-2:

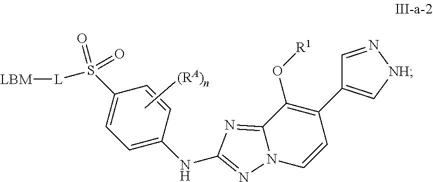

wherein each R¹, R^{A}, L and LBM are as defined in any of the embodiments described herein.

In an embodiment, provided is a compound of Formula III-b:

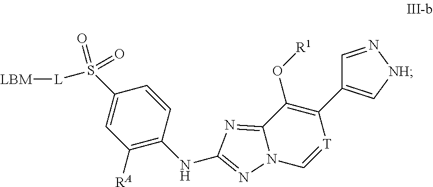

wherein each R¹, T, L and LBM are as defined in any of the embodiments described herein.

In an embodiment, provided is a compound of Formula III-b-1:

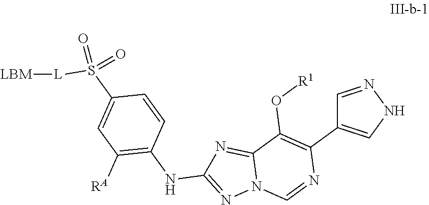

wherein each R¹, L and LBM are as defined in any of the embodiments described herein.

In an embodiment, provided is a compound of Formula III-b-2:

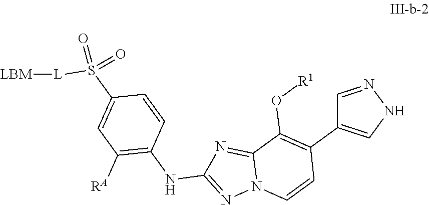

wherein each R¹, L and LBM are as defined in any of the embodiments described herein.

In an embodiment, provided is a compound of Formula III-c:

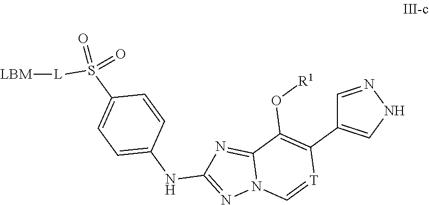

wherein each R¹, T, L and LBM are as defined in any of the embodiments described herein.

In an embodiment, provided is a compound of Formula III-c-1:

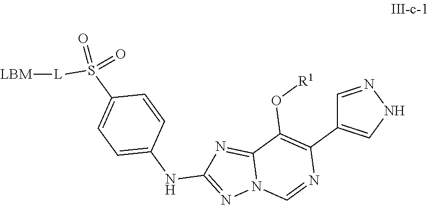

wherein each R¹, L and LBM are as defined in any of the embodiments described herein.

In an embodiment, provided is a compound of Formula III-c-2:

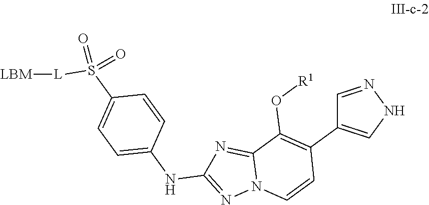

wherein each R¹, L and LBM are as defined in any of the embodiments described herein.

In an embodiment, provided is a compound of Formula IV.

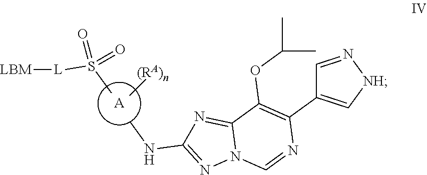

wherein Ring A, R^{A}, n, L and LBM are as defined in any of the embodiments described herein.

In an embodiment, provided is a compound of Formula IV-a.

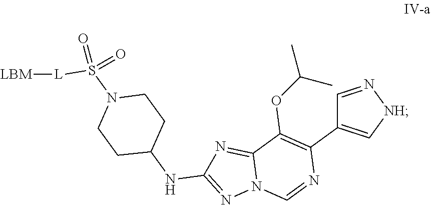

wherein L and LBM are as defined in any of the embodiments described herein.

In an embodiment, provided is a compound of Formula IV-b.

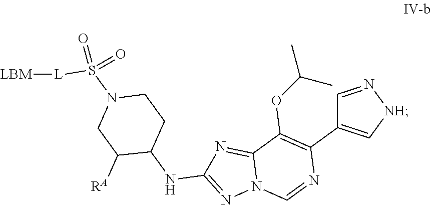

wherein R^{A}, n, L and LBM are as defined in any of the embodiments described herein.

In an embodiment, provided is a compound of Formula IV-c.

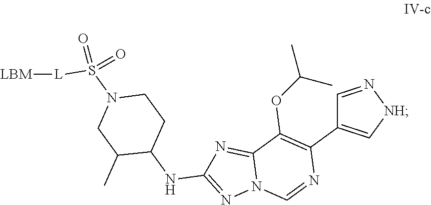

wherein L and LBM are as defined in any of the embodiments described herein.

In an embodiment, provided is a compound of Formula IV-d.

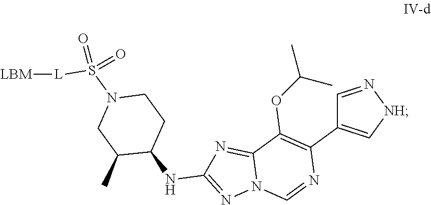

wherein L and LBM are as defined in any of the embodiments described herein.

In an embodiment, provided is a compound of Formula IV-e

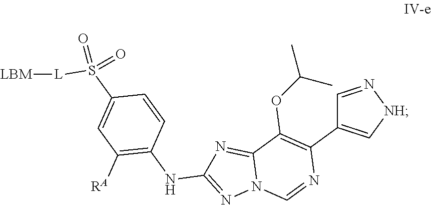

wherein R^{A}, L and LBM are as defined in any of the embodiments described herein.

In an embodiment, provided is a compound of Formula IV-f

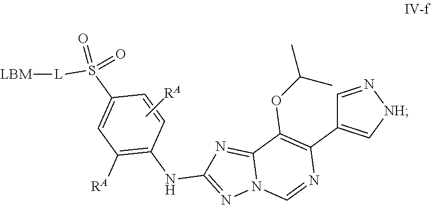

wherein R^{A}, L and LBM are as defined in any of the embodiments described herein.

In an embodiment, provided is a compound of Formula IV-g

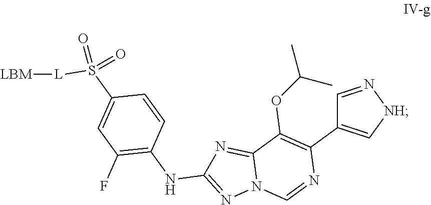

wherein L and LBM are as defined in any of the embodiments described herein.

In an embodiment, provided is a compound of Formula IV-h

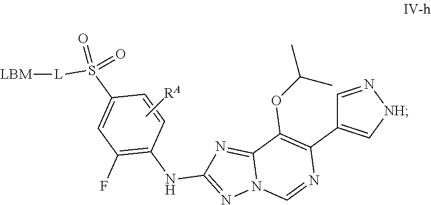

wherein R^{A}, L and LBM are as defined in any of the embodiments described herein.

In an embodiment, provided is a compound of Formula IV-i:

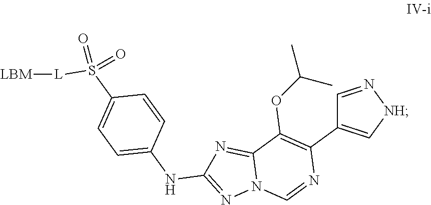

wherein L and LBM are as defined in any of the embodiments described herein.

As generally defined herein, each of W¹, W², W³ and W⁴ is selected from CH or N, provided that no more than two of W¹, W², W³ and W⁴ are N. In one embodiment, W¹ is N and W², W³ and W⁴ are CH. In one embodiment, W² is N and W¹, W³ and W⁴ are CH. In one embodiment, W³ is N and W¹, W² and W⁴ are CH. In one embodiment, W⁴ is N and W¹, W² and W³ are CH. In one embodiment, W¹ and W² are N and W³ and W⁴ are CH. In one embodiment, W¹ and W³ are N and W² and W⁴ are CH. In one embodiment, W¹ and W⁴ are N and W² and W³ are CH. In one embodiment, W² and W³ are N and W¹ and W⁴ are CH. In one embodiment, W² and W⁴ are N and W¹ and W³ are CH. In one embodiment, W³ and W⁴ are N and W¹ and W³ are CH.

In an embodiment of a compound of Formula A-I or Formula I, the compound is selected from the compounds disclosed in Table 1, or a pharmaceutically acceptable salt thereof, or elsewhere in the specification and figures.

In an embodiment, provided herein is a composition comprising a compound described herein and a pharmaceutically acceptable excipient.

In an embodiment, the compound is a compound identified in Table 1 below or a pharmaceutically acceptable salt thereof.

Unless otherwise indicated, the absolute stereochemistry of all chiral atoms is as depicted. Compounds marked with (or) or (rel) in Table 1 and the Examples section are single enantiomers wherein the absolute stereochemistry was arbitrarily assigned (e.g., based on chiral SFC elution as described in the Examples section). Compounds marked with (and) or (rac) are mixtures of enantiomers wherein the relative stereochemistry is as shown. Compounds that have a stereogenic center where the configuration is not indicated in the structure as depicted and that have no designation in the stereochemistry column of Table 1 are mixtures of enantiomers at that center. Compounds that have a stereogenic center where the configuration is indicated in the structure as depicted and have no designation in the stereochemistry column of Table 1 or that are marked with (abs) are single enantiomers wherein the absolute stereochemistry is as indicated.

A person of skill in the art would be able to separate racemic compounds into the respective enantiomers using methods known in the art, such as chiral chromatography, chiral recrystallization and the like. References to compounds that are racemic mixtures are meant to also include the individual enantiomers contained in the mixture.

TABLE 1
Exemplary compounds

| Structure | Nr |
|---|---|
| 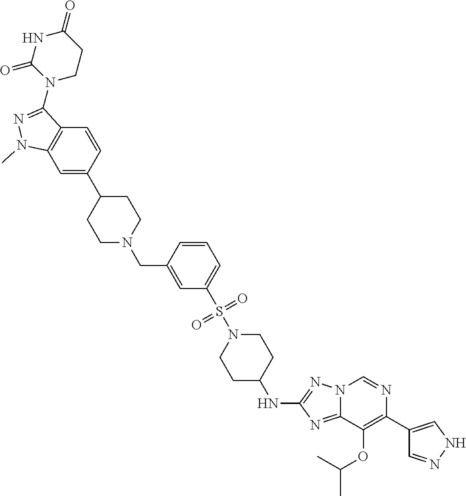 [SMILES: CC(C)Oc1c(-c2cn[nH]c2)ncn2nc(NC3CCN(S(=O)(=O)c4cccc(CN5CCC(c6ccc7c(N8CCC(=O)NC8=O)nn(C)c7c6)CC5)c4)CC3)nc12] | 1 |
| 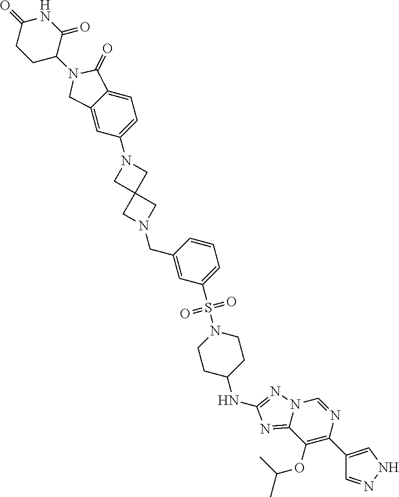 [SMILES: CC(C)Oc1c(-c2cn[nH]c2)ncn2nc(NC3CCN(S(=O)(=O)c4cccc(CN5CC6(C5)CN(c5ccc7c(c5)CN(C5CCC(=O)NC5=O)C7=O)C6)c4)CC3)nc12] | 2 |
| 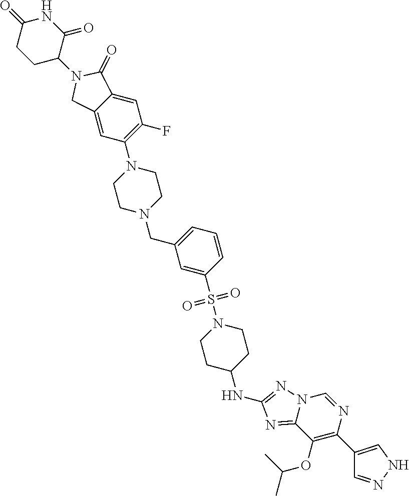 [SMILES: CC(C)Oc1c(-c2cn[nH]c2)ncn2nc(NC3CCN(S(=O)(=O)c4cccc(CN5CCN(c6cc7c(cc6F)C(=O)N(C6CCC(=O)NC6=O)C7)CC5)c4)CC3)nc12] | 3 |
| 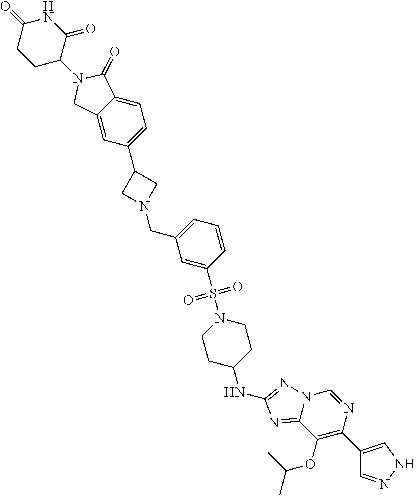 [SMILES: CC(C)Oc1c(-c2cn[nH]c2)ncn2nc(NC3CCN(S(=O)(=O)c4cccc(CN5CC(c6ccc7c(c6)CN(C6CCC(=O)NC6=O)C7=O)C5)c4)CC3)nc12] | 4 |
| 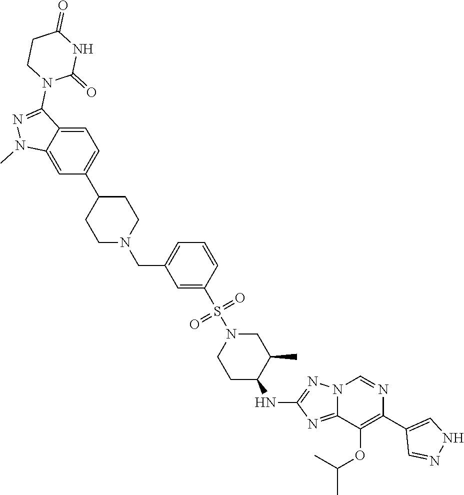 [SMILES: CC(C)Oc1c(-c2cn[nH]c2)ncn2nc(N[C@H]3CCN(S(=O)(=O)c4cccc(CN5CCC(c6ccc7c(N8CCC(=O)NC8=O)nn(C)c7c6)CC5)c4)C[C@H]3C)nc12] | 5 |
| 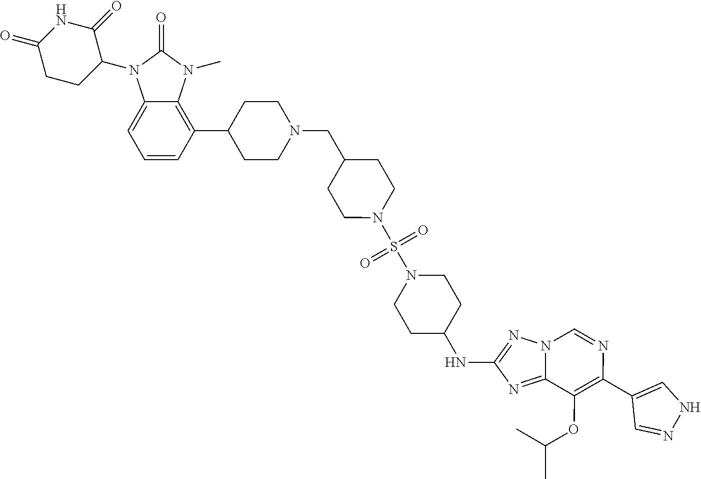 [SMILES: CC(C)Oc1c(-c2cn[nH]c2)ncn2nc(NC3CCN(S(=O)(=O)N4CCC(CN5CCC(c6cccc7c6n(C)c(=O)n7C6CCC(=O)NC6=O)CC5)CC4)CC3)nc12] | 6 |
| 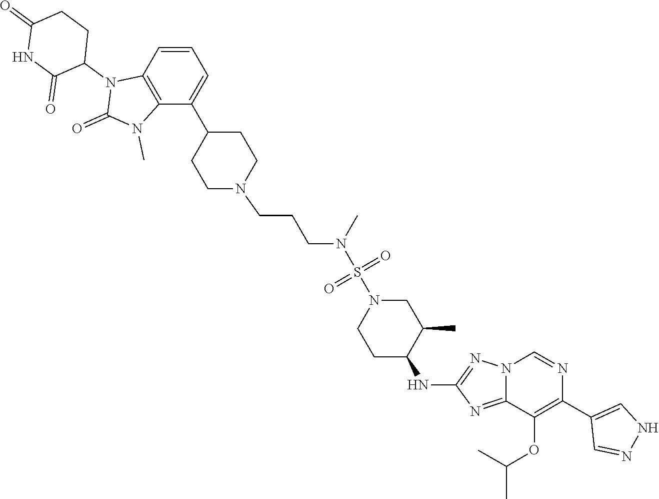 [SMILES: CC(C)Oc1c(-c2cn[nH]c2)ncn2nc(N[C@H]3CCN(S(=O)(=O)N(C)CCCN4CCC(c5cccc6c5n(C)c(=O)n6C5CCC(=O)NC5=O)CC4)C[C@H]3C)nc12] | 7 |
| 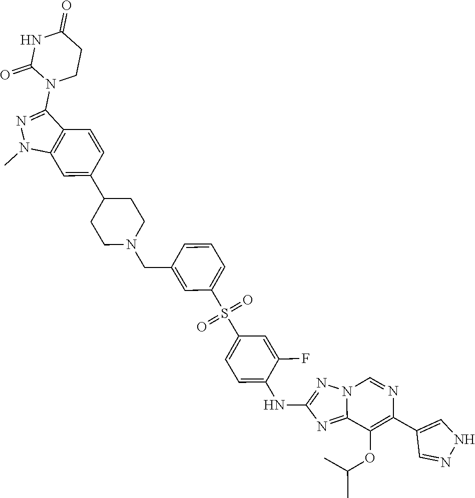 [SMILES: CC(C)Oc1c(-c2cn[nH]c2)ncn2nc(Nc3ccc(S(=O)(=O)c4cccc(CN5CCC(c6ccc7c(N8CCC(=O)NC8=O)nn(C)c7c6)CC5)c4)cc3F)nc12] | 8 |
| 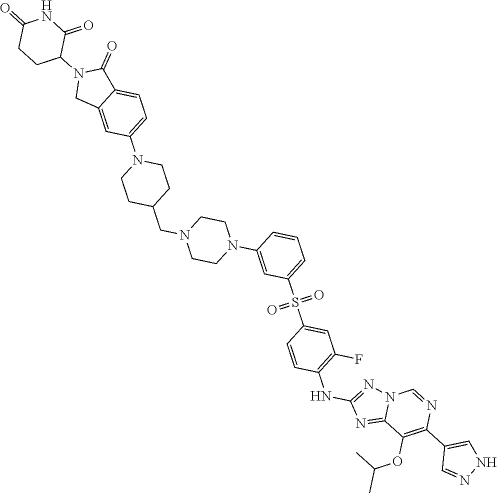 [SMILES: CC(C)Oc1c(-c2cn[nH]c2)ncn2nc(Nc3ccc(S(=O)(=O)c4cccc(N5CCN(CC6CCN(c7ccc8c(c7)CN(C7CCC(=O)NC7=O)C8=O)CC6)CC5)c4)cc3F)nc12] | 9 |
| 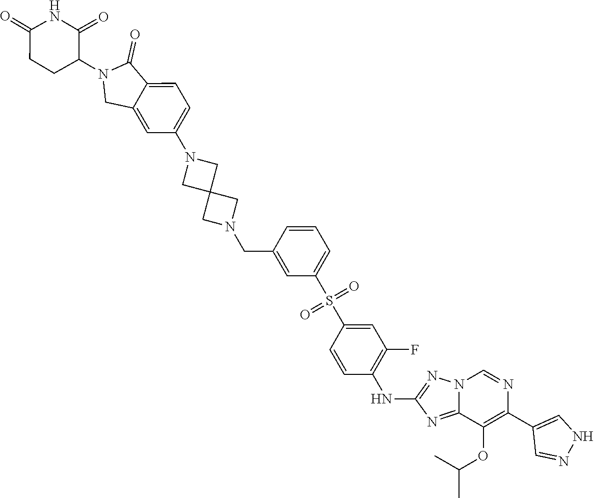 [SMILES: CC(C)Oc1c(-c2cn[nH]c2)ncn2nc(Nc3ccc(S(=O)(=O)c4cccc(CN5CC6(C5)CN(c5ccc7c(c5)CN(C5CCC(=O)NC5=O)C7=O)C6)c4)cc3F)nc12] | 10 |
| 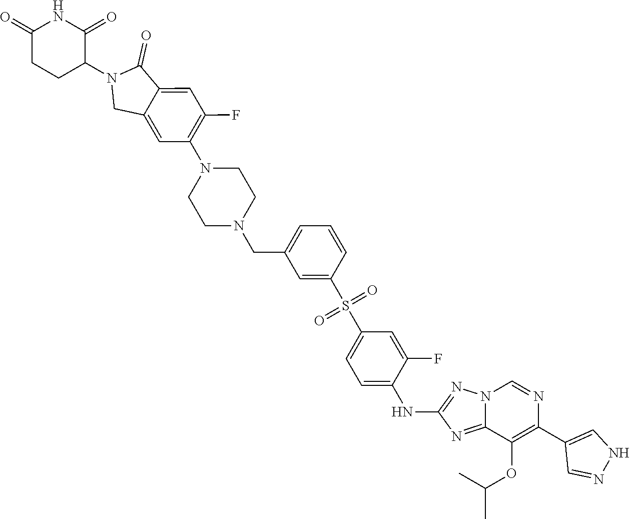 [SMILES: CC(C)Oc1c(-c2cn[nH]c2)ncn2nc(Nc3ccc(S(=O)(=O)c4cccc(CN5CCN(c6cc7c(cc6F)C(=O)N(C6CCC(=O)NC6=O)C7)CC5)c4)cc3F)nc12] | 11 |
| 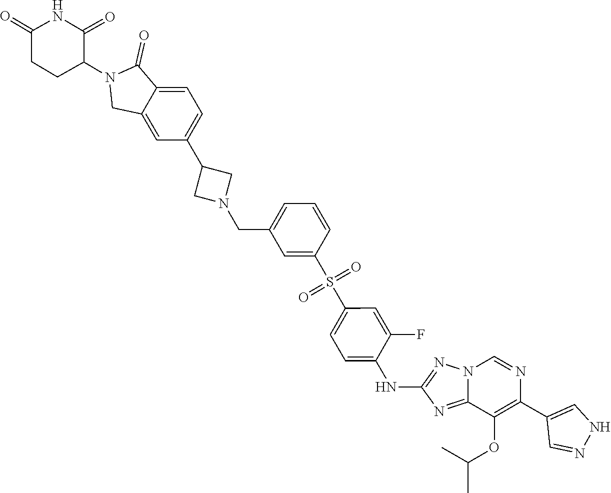 [SMILES: CC(C)Oc1c(-c2cn[nH]c2)ncn2nc(Nc3ccc(S(=O)(=O)c4cccc(CN5CC(c6ccc7c(c6)CN(C6CCC(=O)NC6=O)C7=O)C5)c4)cc3F)nc12] | 12 |
| 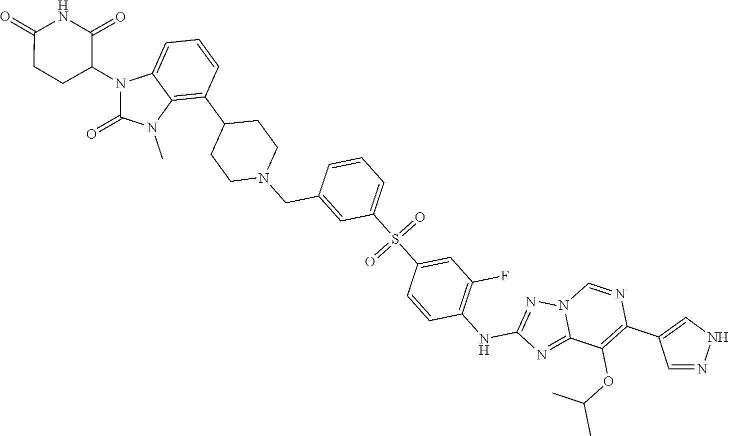 [SMILES: CC(C)Oc1c(-c2cn[nH]c2)ncn2nc(Nc3ccc(S(=O)(=O)c4cccc(CN5CCC(c6cccc7c6n(C)c(=O)n7C6CCC(=O)NC6=O)CC5)c4)cc3F)nc12] | 13 |
| 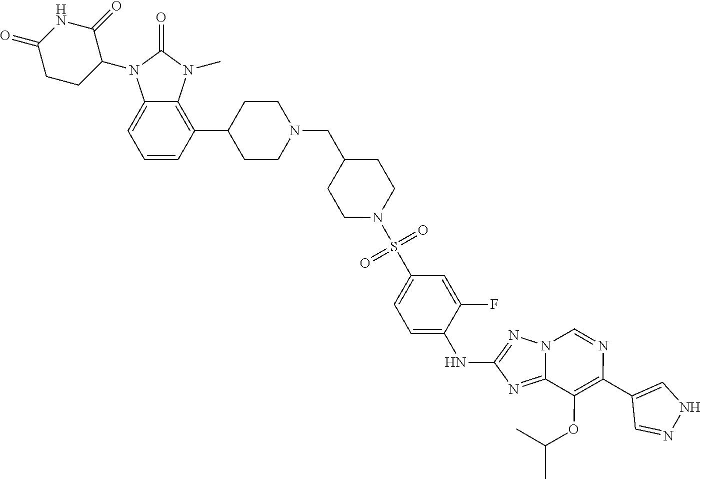 [SMILES: CC(C)Oc1c(-c2cn[nH]c2)ncn2nc(Nc3ccc(S(=O)(=O)N4CCC(CN5CCC(c6cccc7c6n(C)c(=O)n7C6CCC(=O)NC6=O)CC5)CC4)cc3F)nc12] | 14 |
| 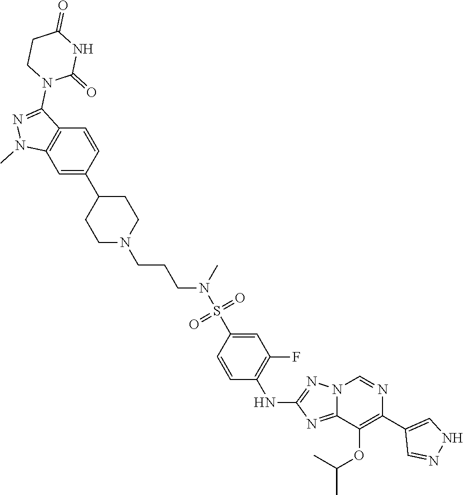 [SMILES: CC(C)Oc1c(-c2cn[nH]c2)ncn2nc(Nc3ccc(S(=O)(=O)N(C)CCCN4CCC(c5ccc6c(N7CCC(=O)NC7=O)nn(C)c6c5)CC4)cc3F)nc12] | 15 |
| 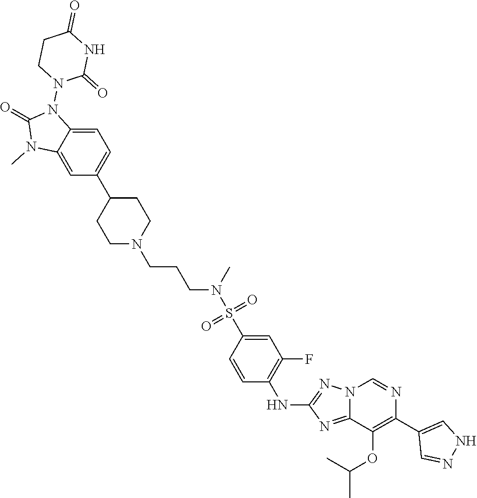 [SMILES: CC(C)Oc1c(-c2cn[nH]c2)ncn2nc(Nc3ccc(S(=O)(=O)N(C)CCCN4CCC(c5ccc6c(c5)n(C)c(=O)n6N5CCC(=O)NC5=O)CC4)cc3F)nc12] | 16 |
| 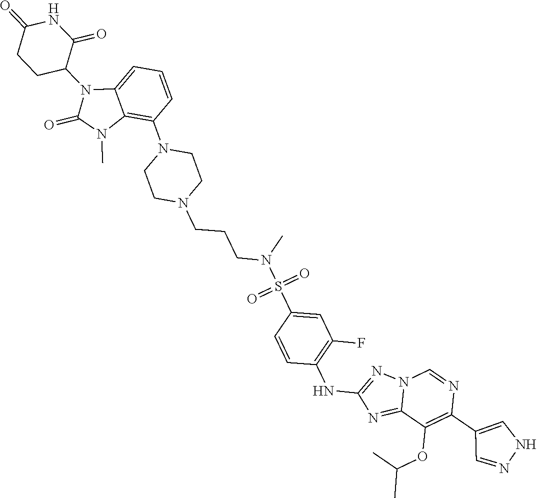 [SMILES: CC(C)Oc1c(-c2cn[nH]c2)ncn2nc(Nc3ccc(S(=O)(=O)N(C)CCCN4CCN(c5cccc6c5n(C)c(=O)n6C5CCC(=O)NC5=O)CC4)cc3F)nc12] | 17 |
| 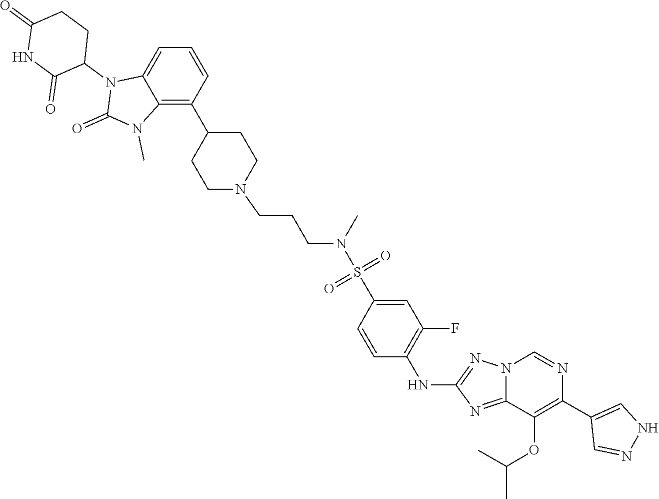 [SMILES: CC(C)Oc1c(-c2cn[nH]c2)ncn2nc(Nc3ccc(S(=O)(=O)N(C)CCCN4CCC(c5cccc6c5n(C)c(=O)n6C5CCC(=O)NC5=O)CC4)cc3F)nc12] | 18 |
| 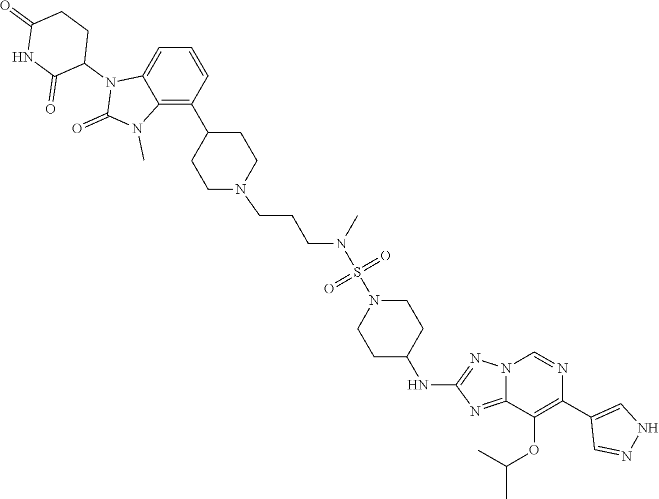 [SMILES: CC(C)Oc1c(-c2cn[nH]c2)ncn2nc(NC3CCN(S(=O)(=O)N(C)CCCN4CCC(c5cccc6c5n(C)c(=O)n6C5CCC(=O)NC5=O)CC4)CC3)nc12] | 19 |
| 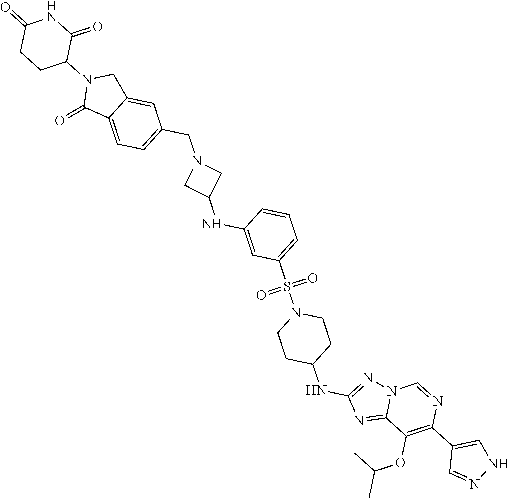 [SMILES: CC(C)Oc1c(-c2cn[nH]c2)ncn2nc(NC3CCN(S(=O)(=O)c4cccc(NC5CN(Cc6ccc7c(c6)CN(C6CCC(=O)NC6=O)C7=O)C5)c4)CC3)nc12] | 20 |
| 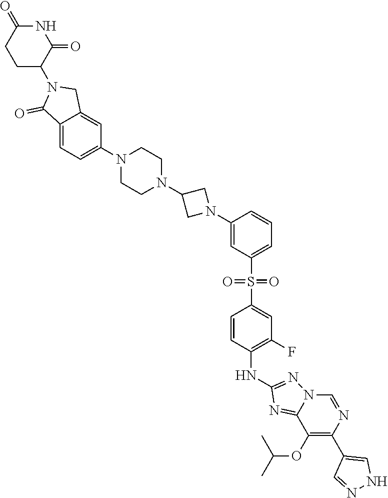 [SMILES: CC(C)Oc1c(-c2cn[nH]c2)ncn2nc(Nc3ccc(S(=O)(=O)c4cccc(N5CC(N6CCN(c7ccc8c(c7)CN(C7CCC(=O)NC7=O)C8=O)CC6)C5)c4)cc3F)nc12] | 21 |
| 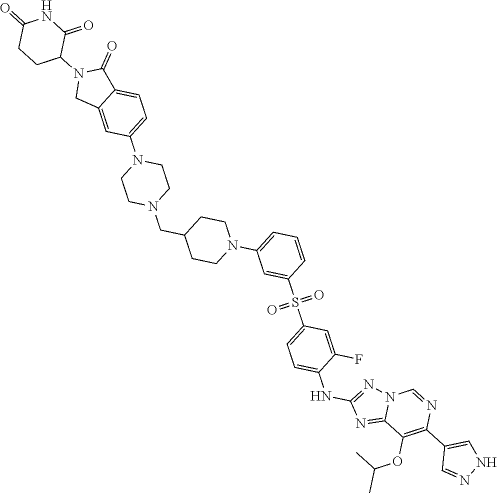 [SMILES: CC(C)Oc1c(-c2cn[nH]c2)ncn2nc(Nc3ccc(S(=O)(=O)c4cccc(N5CCC(CN6CCN(c7ccc8c(c7)CN(C7CCC(=O)NC7=O)C8=O)CC6)CC5)c4)cc3F)nc12] | 22 |
| 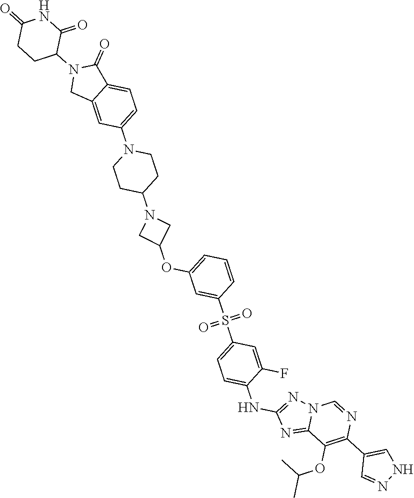 [SMILES: CC(C)Oc1c(-c2cn[nH]c2)ncn2nc(Nc3ccc(S(=O)(=O)c4cccc(OC5CN(C6CCN(c7ccc8c(c7)CN(C7CCC(=O)NC7=O)C8=O)CC6)C5)c4)cc3F)nc12] | 23 |
| 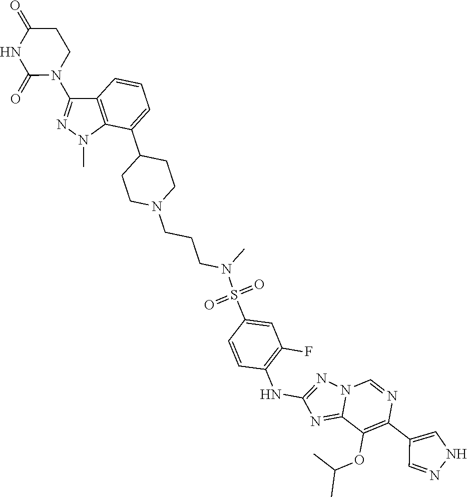 [SMILES: CC(C)Oc1c(-c2cn[nH]c2)ncn2nc(Nc3ccc(S(=O)(=O)N(C)CCCN4CCC(c5cccc6c(N7CCC(=O)NC7=O)nn(C)c56)CC4)cc3F)nc12] | 24 |
| 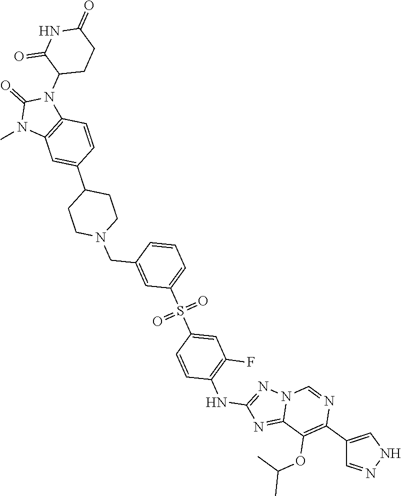 [SMILES: CC(C)Oc1c(-c2cn[nH]c2)ncn2nc(Nc3ccc(S(=O)(=O)c4cccc(CN5CCC(c6ccc7c(c6)n(C)c(=O)n7C6CCC(=O)NC6=O)CC5)c4)cc3F)nc12] | 25 |
| 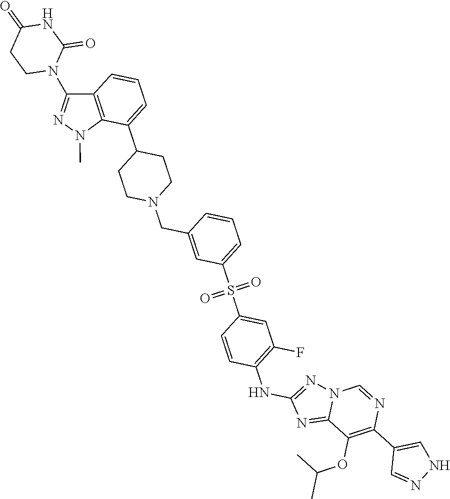 [SMILES: CC(C)Oc1c(-c2cn[nH]c2)ncn2nc(Nc3ccc(S(=O)(=O)c4cccc(CN5CCC(c6cccc7c(N8CCC(=O)NC8=O)nn(C)c67)CC5)c4)cc3F)nc12] | 26 |
| 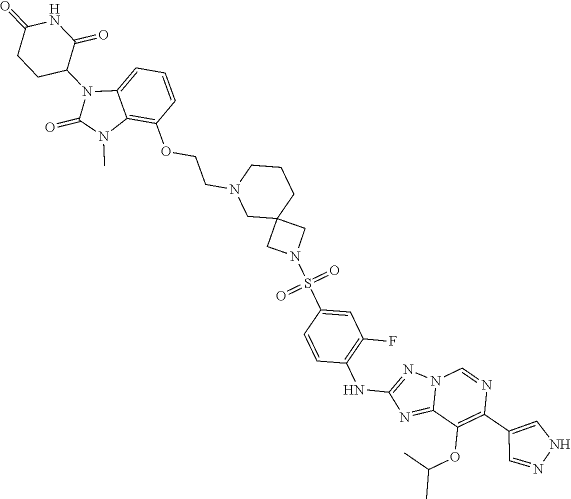 [SMILES: CC(C)Oc1c(-c2cn[nH]c2)ncn2nc(Nc3ccc(S(=O)(=O)N4CC5(CCCN(CCOc6cccc7c6n(C)c(=O)n7C6CCC(=O)NC6=O)C5)C4)cc3F)nc12] | 27 |
| 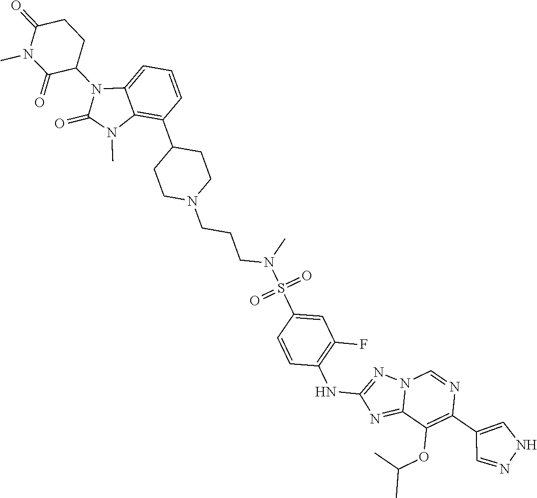 [SMILES: CC(C)Oc1c(-c2cn[nH]c2)ncn2nc(Nc3ccc(S(=O)(=O)N(C)CCCN4CCC(c5cccc6c5n(C)c(=O)n6C5CCC(=O)N(C)C5=O)CC4)cc3F)nc12] | 28 |
| 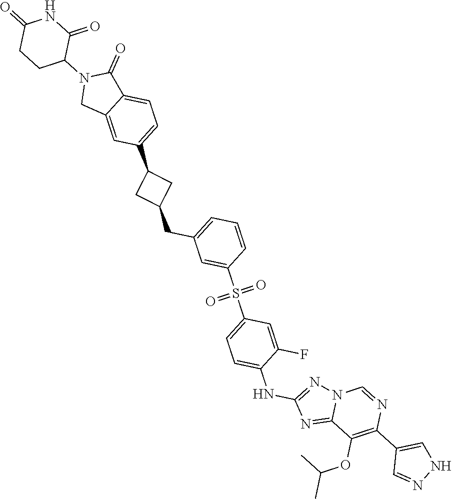 [SMILES: CC(C)Oc1c(-c2cn[nH]c2)ncn2nc(Nc3ccc(S(=O)(=O)c4cccc(C[C@H]5C[C@@H](c6ccc7c(c6)CN(C6CCC(=O)NC6=O)C7=O)C5)c4)cc3F)nc12] | 29 |
| 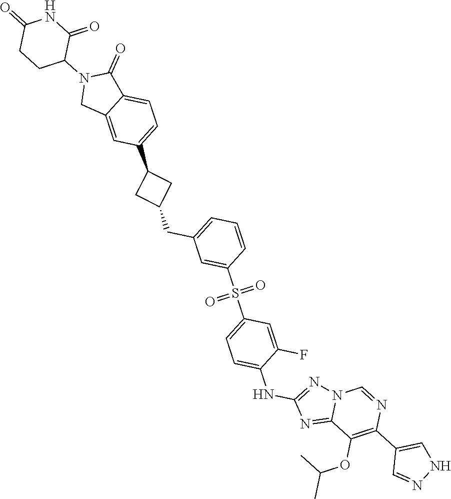 [SMILES: CC(C)Oc1c(-c2cn[nH]c2)ncn2nc(Nc3ccc(S(=O)(=O)c4cccc(C[C@H]5C[C@H](c6ccc7c(c6)CN(C6CCC(=O)NC6=O)C7=O)C5)c4)cc3F)nc12] | 30 |
| 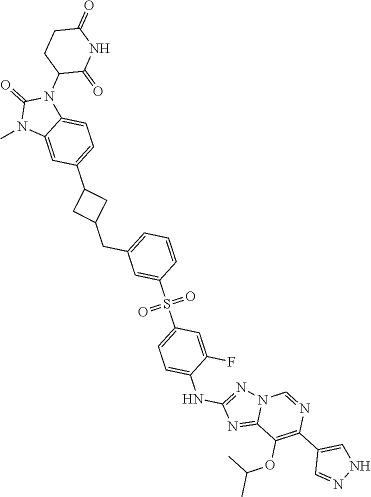 [SMILES: CC(C)Oc1c(-c2cn[nH]c2)ncn2nc(Nc3ccc(S(=O)(=O)c4cccc(CC5CC(c6ccc7c(c6)n(C)c(=O)n7C6CCC(=O)NC6=O)C5)c4)cc3F)nc12] | 31 |
| 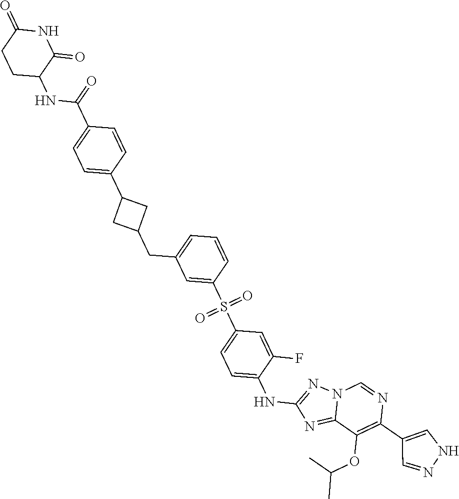 [SMILES: CC(C)Oc1c(-c2cn[nH]c2)ncn2nc(Nc3ccc(S(=O)(=O)c4cccc(CC5CC(c6ccc(C(=O)NC7CCC(=O)NC7=O)cc6)C5)c4)cc3F)nc12] | 32 |
| 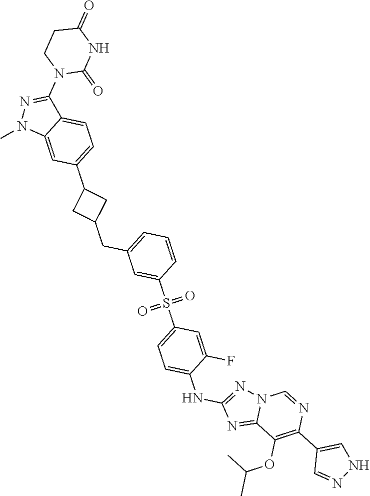 [SMILES: CC(C)Oc1c(-c2cn[nH]c2)ncn2nc(Nc3ccc(S(=O)(=O)c4cccc(CC5CC(c6ccc7c(N8CCC(=O)NC8=O)nn(C)c7c6)C5)c4)cc3F)nc12] | 33 |
| 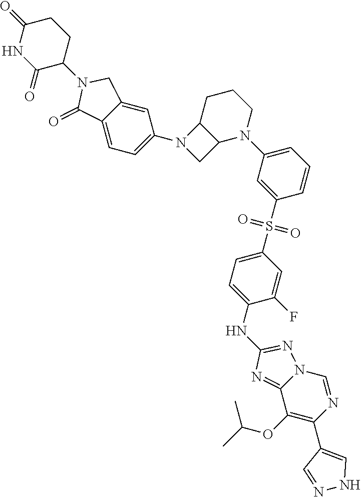 [SMILES: CC(C)Oc1c(-c2cn[nH]c2)ncn2nc(Nc3ccc(S(=O)(=O)c4cccc(N5CCCC6C5CN6c5ccc6c(c5)CN(C5CCC(=O)NC5=O)C6=O)c4)cc3F)nc12] | 34 |
| 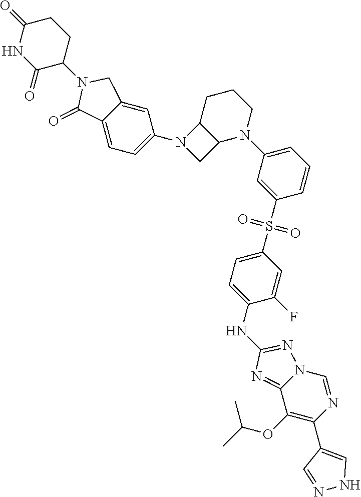 [SMILES: CC(C)Oc1c(-c2cn[nH]c2)ncn2nc(Nc3ccc(S(=O)(=O)c4cccc(N5CCCC6C5CN6c5ccc6c(c5)CN(C5CCC(=O)NC5=O)C6=O)c4)cc3F)nc12] | 35 |
| 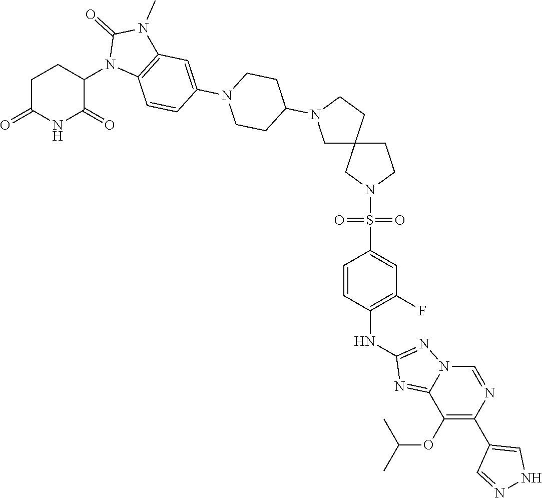 [SMILES: CC(C)Oc1c(-c2cn[nH]c2)ncn2nc(Nc3ccc(S(=O)(=O)N4CCC5(CCN(C6CCN(c7ccc8c(c7)n(C)c(=O)n8C7CCC(=O)NC7=O)CC6)C5)C4)cc3F)nc12] | 36 |
| 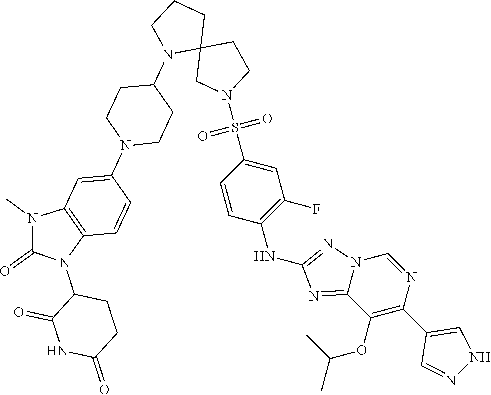 [SMILES: CC(C)Oc1c(-c2cn[nH]c2)ncn2nc(Nc3ccc(S(=O)(=O)N4CCC5(CCCN5C5CCN(c6ccc7c(c6)n(C)c(=O)n7C6CCC(=O)NC6=O)CC5)C4)cc3F)nc12] | 37 |
| 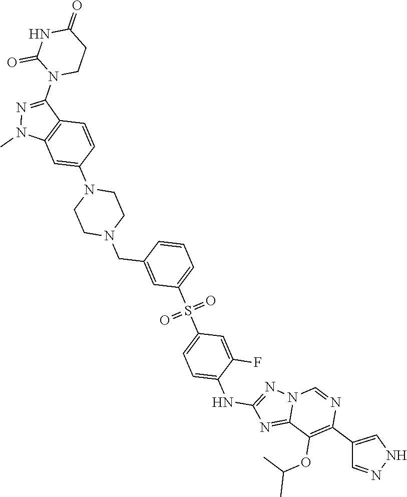 [SMILES: CC(C)Oc1c(-c2cn[nH]c2)ncn2nc(Nc3ccc(S(=O)(=O)c4cccc(CN5CCN(c6ccc7c(N8CCC(=O)NC8=O)nn(C)c7c6)CC5)c4)cc3F)nc12] | 38 |
| 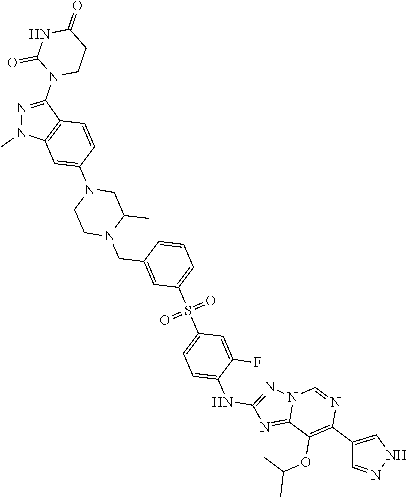 [SMILES: CC(C)Oc1c(-c2cn[nH]c2)ncn2nc(Nc3ccc(S(=O)(=O)c4cccc(CN5CCN(c6ccc7c(N8CCC(=O)NC8=O)nn(C)c7c6)CC5C)c4)cc3F)nc12] | 39 |
| 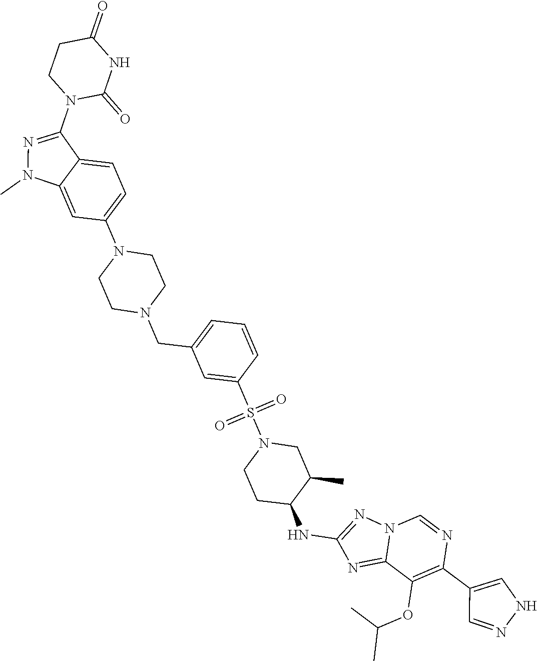 [SMILES: CC(C)Oc1c(-c2cn[nH]c2)ncn2nc(N[C@H]3CCN(S(=O)(=O)c4cccc(CN5CCN(c6ccc7c(N8CCC(=O)NC8=O)nn(C)c7c6)CC5)c4)C[C@H]3C)nc12] | 40 |
| 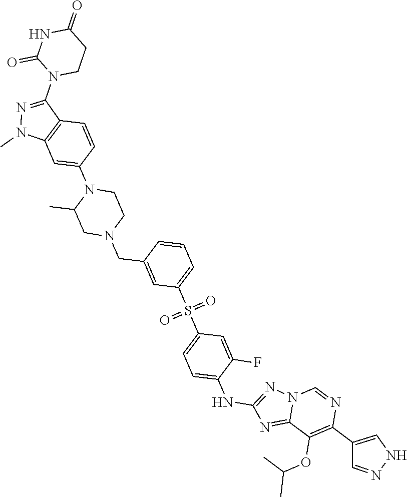 [SMILES: CC(C)Oc1c(-c2cn[nH]c2)ncn2nc(Nc3ccc(S(=O)(=O)c4cccc(CN5CCN(c6ccc7c(N8CCC(=O)NC8=O)nn(C)c7c6)C(C)C5)c4)cc3F)nc12] | 41 |
| 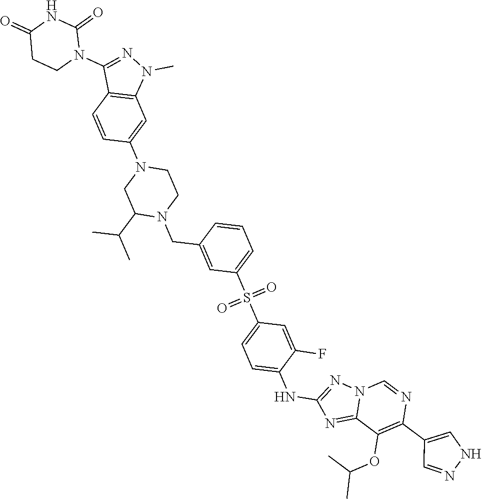 [SMILES: CC(C)Oc1c(-c2cn[nH]c2)ncn2nc(Nc3ccc(S(=O)(=O)c4cccc(CN5CCN(c6ccc7c(N8CCC(=O)NC8=O)nn(C)c7c6)CC5C(C)C)c4)cc3F)nc12] | 42 |
| 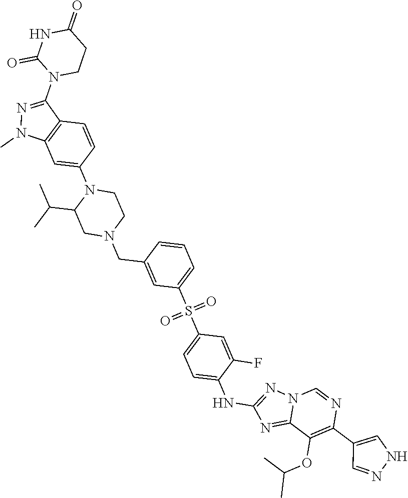 [SMILES: CC(C)Oc1c(-c2cn[nH]c2)ncn2nc(Nc3ccc(S(=O)(=O)c4cccc(CN5CCN(c6ccc7c(N8CCC(=O)NC8=O)nn(C)c7c6)C(C(C)C)C5)c4)cc3F)nc12] | 43 |
| 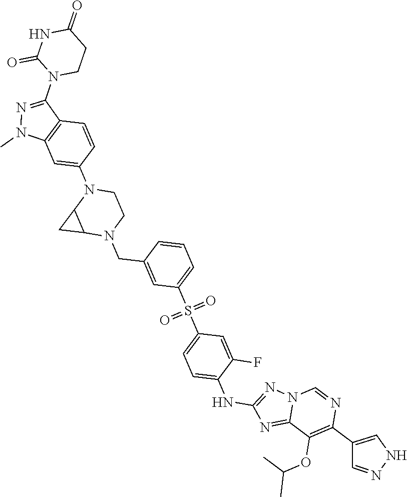 [SMILES: CC(C)Oc1c(-c2cn[nH]c2)ncn2nc(Nc3ccc(S(=O)(=O)c4cccc(CN5CCN(c6ccc7c(N8CCC(=O)NC8=O)nn(C)c7c6)C6CC65)c4)cc3F)nc12] | 44 |
| 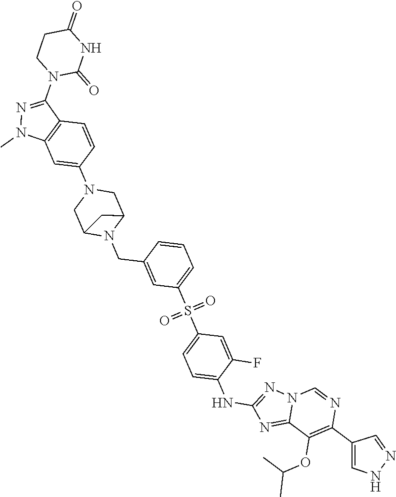 [SMILES: CC(C)Oc1c(-c2cn[nH]c2)ncn2nc(Nc3ccc(S(=O)(=O)c4cccc(CN5C6CC5CN(c5ccc7c(N8CCC(=O)NC8=O)nn(C)c7c5)C6)c4)cc3F)nc12] | 45 |
| 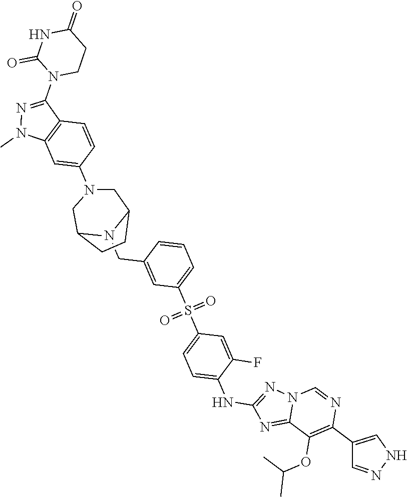 [SMILES: CC(C)Oc1c(-c2cn[nH]c2)ncn2nc(Nc3ccc(S(=O)(=O)c4cccc(CN5C6CCC5CN(c5ccc7c(N8CCC(=O)NC8=O)nn(C)c7c5)C6)c4)cc3F)nc12] | 46 |
| 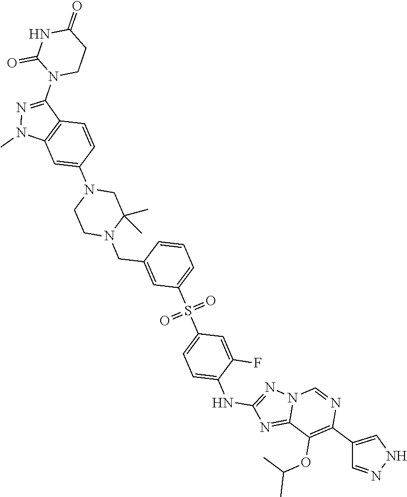 [SMILES: CC(C)Oc1c(-c2cn[nH]c2)ncn2nc(Nc3ccc(S(=O)(=O)c4cccc(CN5CCN(c6ccc7c(N8CCC(=O)NC8=O)nn(C)c7c6)CC5(C)C)c4)cc3F)nc12] | 47 |
| 4 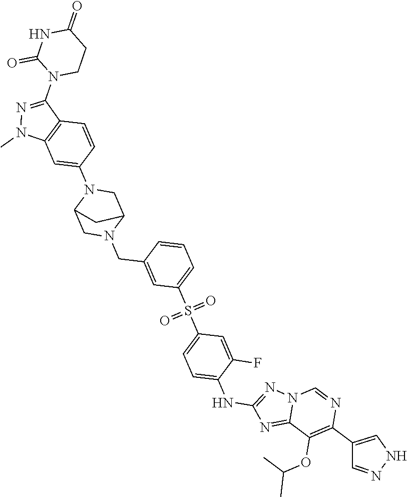 [SMILES: CC(C)Oc1c(-c2cn[nH]c2)ncn2nc(Nc3ccc(S(=O)(=O)c4cccc(CN5CC6CC5CN6c5ccc6c(N7CCC(=O)NC7=O)nn(C)c6c5)c4)cc3F)nc12] | 48 |
| 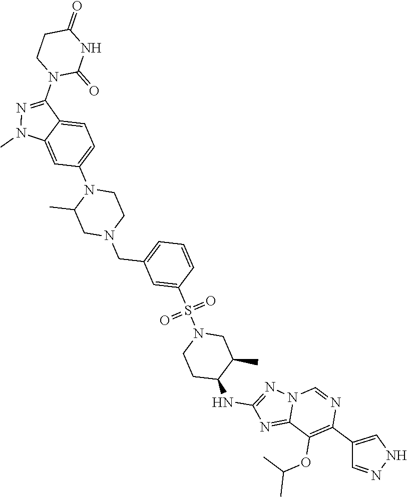 [SMILES: CC(C)Oc1c(-c2cn[nH]c2)ncn2nc(N[C@H]3CCN(S(=O)(=O)c4cccc(CN5CCN(c6ccc7c(N8CCC(=O)NC8=O)nn(C)c7c6)C(C)C5)c4)C[C@H]3C)nc12] | 49 |
| 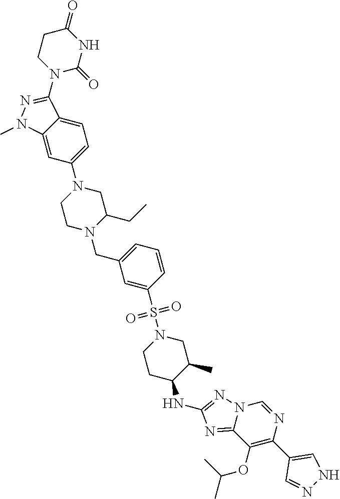 [SMILES: CCC1CN(c2ccc3c(N4CCC(=O)NC4=O)nn(C)c3c2)CCN1Cc1cccc(S(=O)(=O)N2CC[C@H](Nc3nc4c(OC(C)C)c(-c5cn[nH]c5)ncn4n3)[C@H](C)C2)c1] | 50 |
| 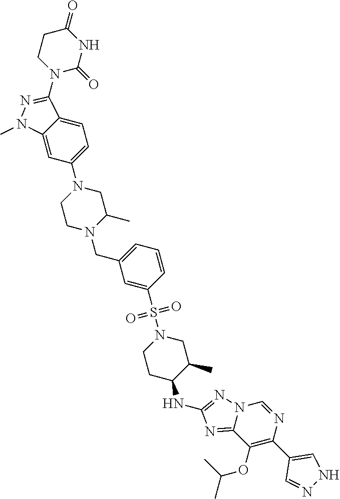 [SMILES: CC(C)Oc1c(-c2cn[nH]c2)ncn2nc(N[C@H]3CCN(S(=O)(=O)c4cccc(CN5CCN(c6ccc7c(N8CCC(=O)NC8=O)nn(C)c7c6)CC5C)c4)C[C@H]3C)nc12] | 51 |
| 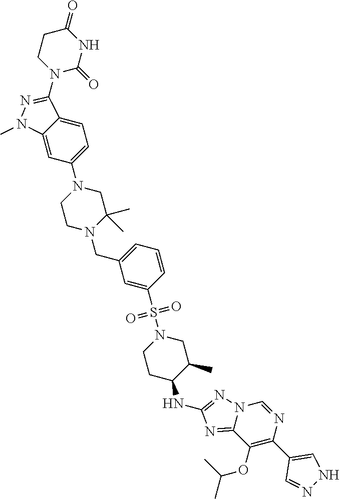 [SMILES: CC(C)Oc1c(-c2cn[nH]c2)ncn2nc(N[C@H]3CCN(S(=O)(=O)c4cccc(CN5CCN(c6ccc7c(N8CCC(=O)NC8=O)nn(C)c7c6)CC5(C)C)c4)C[C@H]3C)nc12] | 52 |
| 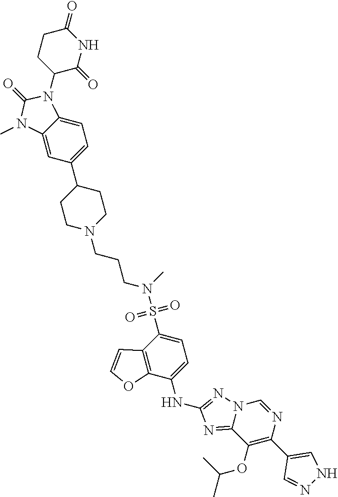 [SMILES: CC(C)Oc1c(-c2cn[nH]c2)ncn2nc(Nc3ccc(S(=O)(=O)N(C)CCCN4CCC(c5ccc6c(c5)n(C)c(=O)n6C5CCC(=O)NC5=O)CC4)c4ccoc34)nc12] | 53 |
| 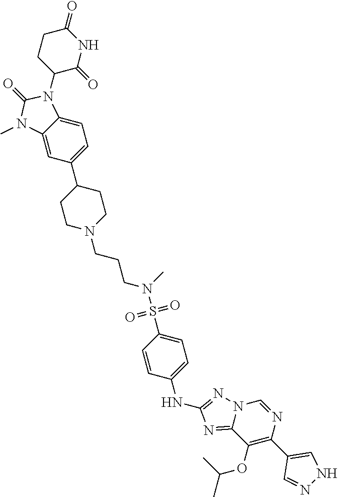 [SMILES: CC(C)Oc1c(-c2cn[nH]c2)ncn2nc(Nc3ccc(S(=O)(=O)N(C)CCCN4CCC(c5ccc6c(c5)n(C)c(=O)n6C5CCC(=O)NC5=O)CC4)cc3)nc12] | 54 |
| 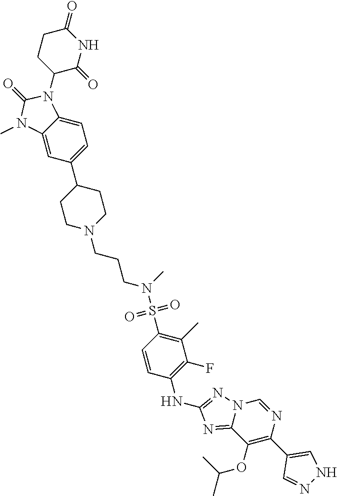 [SMILES: Cc1c(S(=O)(=O)N(C)CCCN2CCC(c3ccc4c(c3)n(C)c(=O)n4C3CCC(=O)NC3=O)CC2)ccc(Nc2nc3c(OC(C)C)c(-c4cn[nH]c4)ncn3n2)c1F] | 55 |
| 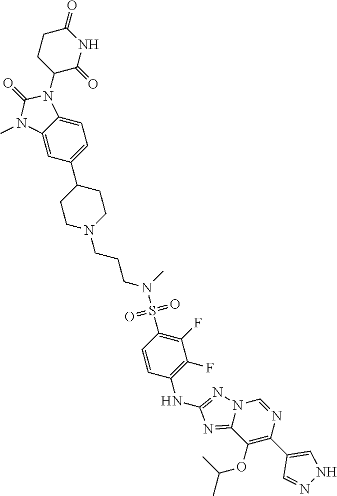 [SMILES: CC(C)Oc1c(-c2cn[nH]c2)ncn2nc(Nc3ccc(S(=O)(=O)N(C)CCCN4CCC(c5ccc6c(c5)n(C)c(=O)n6C5CCC(=O)NC5=O)CC4)c(F)c3F)nc12] | 56 |
| 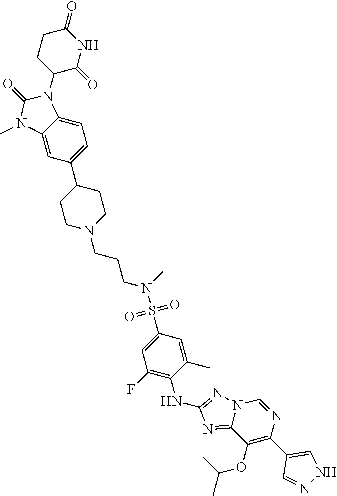 [SMILES: Cc1cc(S(=O)(=O)N(C)CCCN2CCC(c3ccc4c(c3)n(C)c(=O)n4C3CCC(=O)NC3=O)CC2)cc(F)c1Nc1nc2c(OC(C)C)c(-c3cn[nH]c3)ncn2n1] | 57 |
| 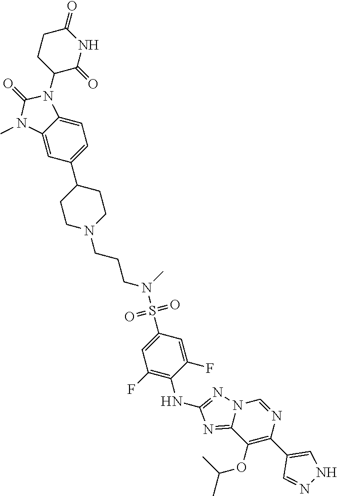 [SMILES: CC(C)Oc1c(-c2cn[nH]c2)ncn2nc(Nc3c(F)cc(S(=O)(=O)N(C)CCCN4CCC(c5ccc6c(c5)n(C)c(=O)n6C5CCC(=O)NC5=O)CC4)cc3F)nc12] | 58 |
| 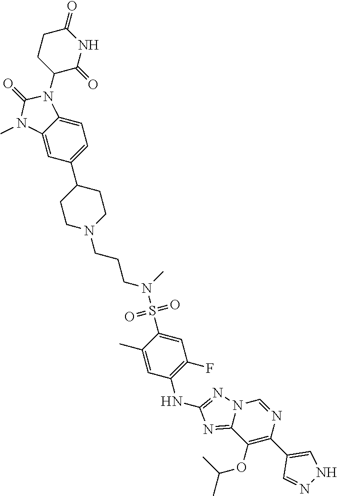 [SMILES: Cc1cc(Nc2nc3c(OC(C)C)c(-c4cn[nH]c4)ncn3n2)c(F)cc1S(=O)(=O)N(C)CCCN1CCC(c2ccc3c(c2)n(C)c(=O)n3C2CCC(=O)NC2=O)CC1] | 59 |
| 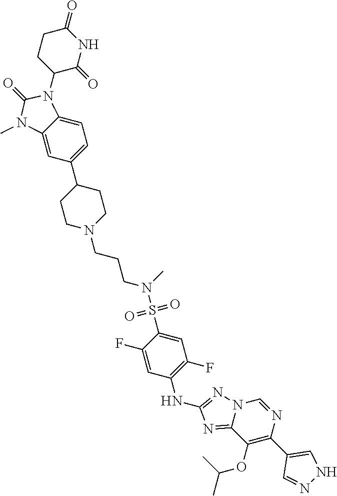 [SMILES: CC(C)Oc1c(-c2cn[nH]c2)ncn2nc(Nc3cc(F)c(S(=O)(=O)N(C)CCCN4CCC(c5ccc6c(c5)n(C)c(=O)n6C5CCC(=O)NC5=O)CC4)cc3F)nc12] | 60 |
| 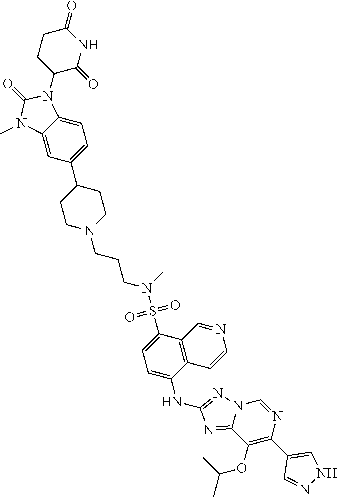 [SMILES: CC(C)Oc1c(-c2cn[nH]c2)ncn2nc(Nc3ccc(S(=O)(=O)N(C)CCCN4CCC(c5ccc6c(c5)n(C)c(=O)n6C5CCC(=O)NC5=O)CC4)c4cnccc34)nc12] | 61 |
| 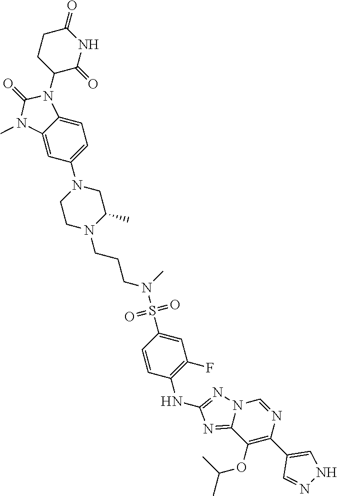 [SMILES: CC(C)Oc1c(-c2cn[nH]c2)ncn2nc(Nc3ccc(S(=O)(=O)N(C)CCCN4CCN(c5ccc6c(c5)n(C)c(=O)n6C5CCC(=O)NC5=O)C[C@@H]4C)cc3F)nc12] | 62 |
| 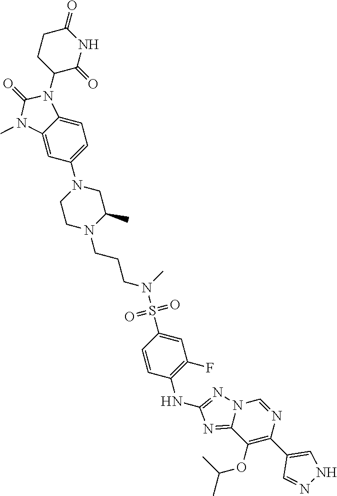 [SMILES: CC(C)Oc1c(-c2cn[nH]c2)ncn2nc(Nc3ccc(S(=O)(=O)N(C)CCCN4CCN(c5ccc6c(c5)n(C)c(=O)n6C5CCC(=O)NC5=O)C[C@H]4C)cc3F)nc12] | 63 |
| 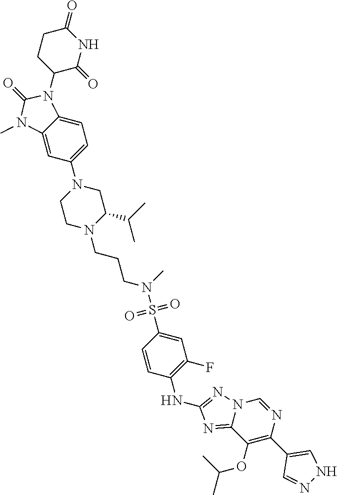 [SMILES: CC(C)Oc1c(-c2cn[nH]c2)ncn2nc(Nc3ccc(S(=O)(=O)N(C)CCCN4CCN(c5ccc6c(c5)n(C)c(=O)n6C5CCC(=O)NC5=O)C[C@@H]4C(C)C)cc3F)nc12] | 64 |
| 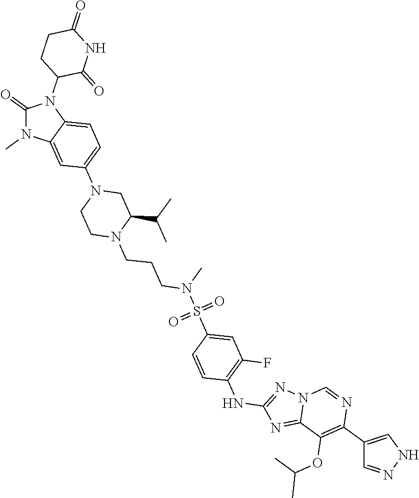 [SMILES: CC(C)Oc1c(-c2cn[nH]c2)ncn2nc(Nc3ccc(S(=O)(=O)N(C)CCCN4CCN(c5ccc6c(c5)n(C)c(=O)n6C5CCC(=O)NC5=O)C[C@H]4C(C)C)cc3F)nc12] | 65 |
| 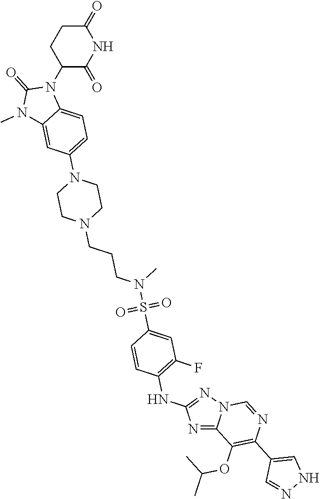 [SMILES: CC(C)Oc1c(-c2cn[nH]c2)ncn2nc(Nc3ccc(S(=O)(=O)N(C)CCCN4CCN(c5ccc6c(c5)n(C)c(=O)n6C5CCC(=O)NC5=O)CC4)cc3F)nc12] | 66 |
| 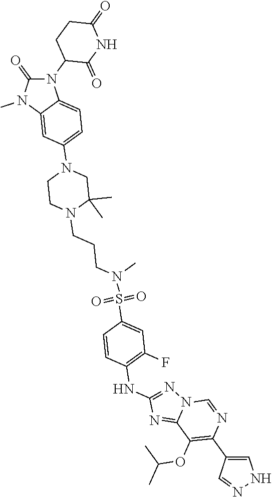 [SMILES: CC(C)Oc1c(-c2cn[nH]c2)ncn2nc(Nc3ccc(S(=O)(=O)N(C)CCCN4CCN(c5ccc6c(c5)n(C)c(=O)n6C5CCC(=O)NC5=O)CC4(C)C)cc3F)nc12] | 67 |
| 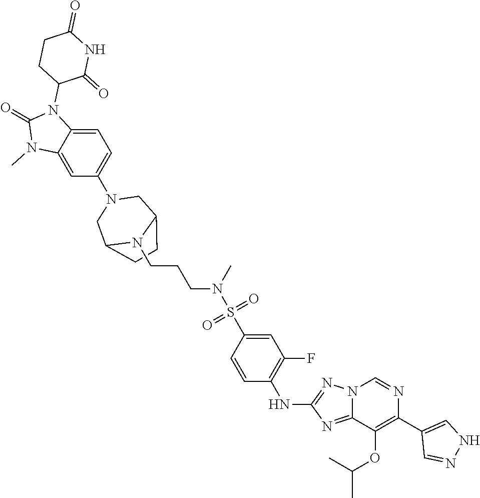 [SMILES: CC(C)Oc1c(-c2cn[nH]c2)ncn2nc(Nc3ccc(S(=O)(=O)N(C)CCCN4C5CCC4CN(c4ccc6c(c4)n(C)c(=O)n6C4CCC(=O)NC4=O)C5)cc3F)nc12] | 68 |
| 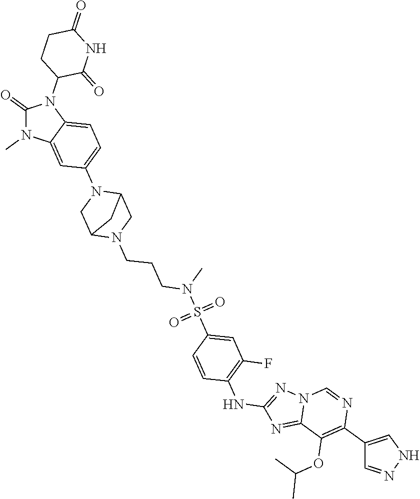 [SMILES: CC(C)Oc1c(-c2cn[nH]c2)ncn2nc(Nc3ccc(S(=O)(=O)N(C)CCCN4CC5CC4CN5c4ccc5c(c4)n(C)c(=O)n5C4CCC(=O)NC4=O)cc3F)nc12] | 69 |
| 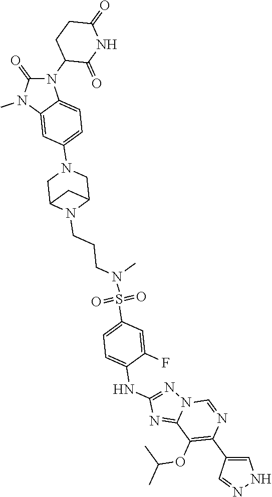 [SMILES: CC(C)Oc1c(-c2cn[nH]c2)ncn2nc(Nc3ccc(S(=O)(=O)N(C)CCCN4C5CC4CN(c4ccc6c(c4)n(C)c(=O)n6C4CCC(=O)NC4=O)C5)cc3F)nc12] | 70 |
| 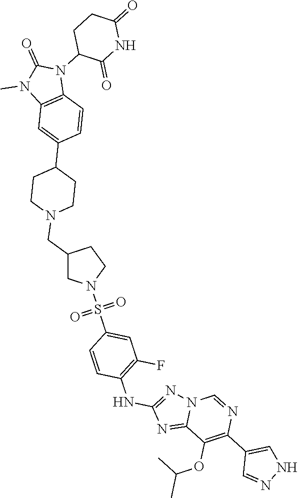 [SMILES: CC(C)Oc1c(-c2cn[nH]c2)ncn2nc(Nc3ccc(S(=O)(=O)N4CCC(CN5CCC(c6ccc7c(c6)n(C)c(=O)n7C6CCC(=O)NC6=O)CC5)C4)cc3F)nc12] | 71 |
| 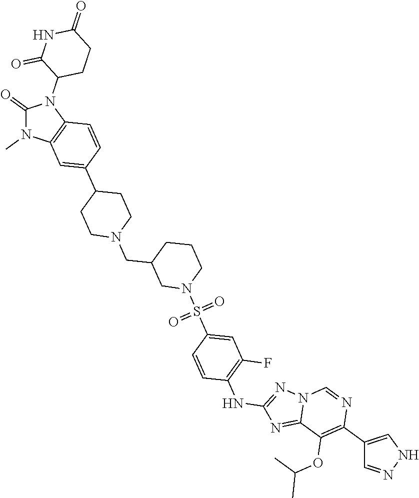 [SMILES: CC(C)Oc1c(-c2cn[nH]c2)ncn2nc(Nc3ccc(S(=O)(=O)N4CCCC(CN5CCC(c6ccc7c(c6)n(C)c(=O)n7C6CCC(=O)NC6=O)CC5)C4)cc3F)nc12] | 72 |
| 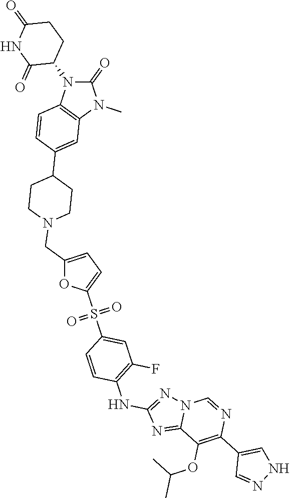 [SMILES: CC(C)Oc1c(-c2cn[nH]c2)ncn2nc(Nc3ccc(S(=O)(=O)c4ccc(CN5CCC(c6ccc7c(c6)n(C)c(=O)n7[C@H]6CCC(=O)NC6=O)CC5)o4)cc3F)nc12] | 73 |
| 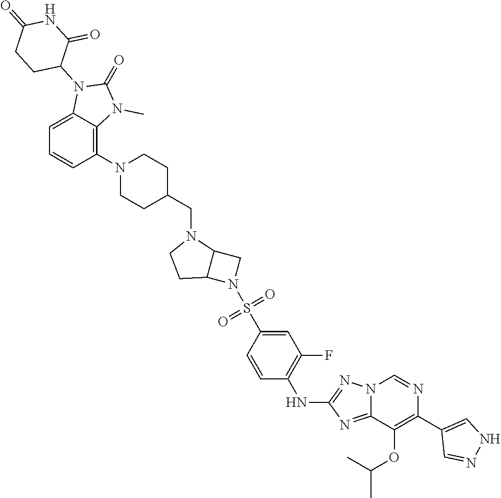 [SMILES: CC(C)Oc1c(-c2cn[nH]c2)ncn2nc(Nc3ccc(S(=O)(=O)N4CC5C4CCN5CC4CCN(c5cccc6c5n(C)c(=O)n6C5CCC(=O)NC5=O)CC4)cc3F)nc12] | 74 |
| 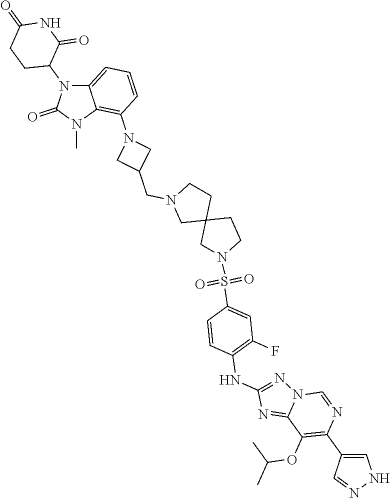 [SMILES: CC(C)Oc1c(-c2cn[nH]c2)ncn2nc(Nc3ccc(S(=O)(=O)N4CCC5(CCN(CC6CN(c7cccc8c7n(C)c(=O)n8C7CCC(=O)NC7=O)C6)C5)C4)cc3F)nc12] | 75 |
| 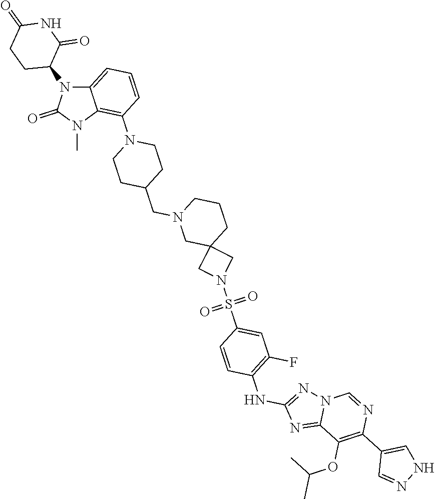 [SMILES: CC(C)Oc1c(-c2cn[nH]c2)ncn2nc(Nc3ccc(S(=O)(=O)N4CC5(CCCN(CC6CCN(c7cccc8c7n(C)c(=O)n8[C@H]7CCC(=O)NC7=O)CC6)C5)C4)cc3F)nc12] | 76 |
| 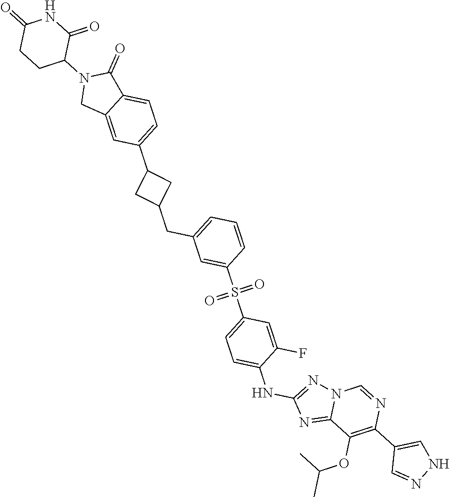 [SMILES: CC(C)Oc1c(-c2cn[nH]c2)ncn2nc(Nc3ccc(S(=O)(=O)c4cccc(CC5CC(c6ccc7c(c6)CN(C6CCC(=O)NC6=O)C7=O)C5)c4)cc3F)nc12] | 77 |
| 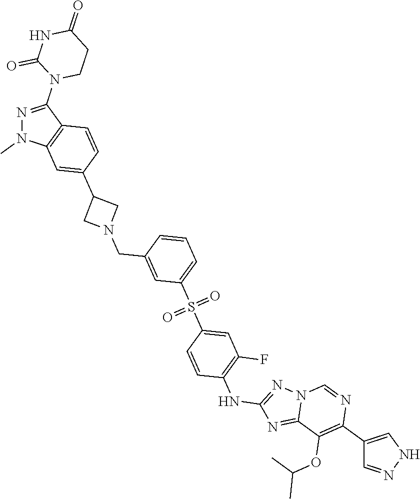 [SMILES: CC(C)Oc1c(-c2cn[nH]c2)ncn2nc(Nc3ccc(S(=O)(=O)c4cccc(CN5CC(c6ccc7c(N8CCC(=O)NC8=O)nn(C)c7c6)C5)c4)cc3F)nc12] | 78 |
| 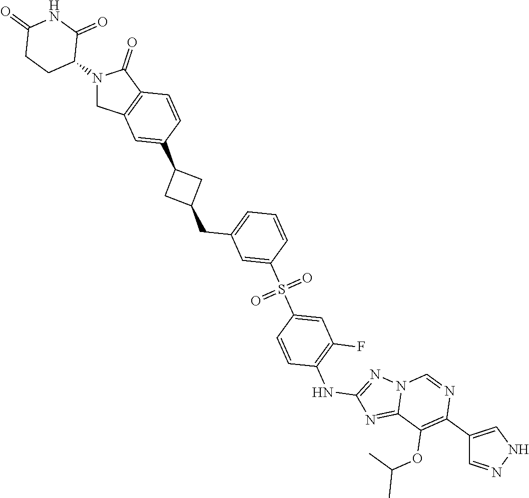 [SMILES: CC(C)Oc1c(-c2cn[nH]c2)ncn2nc(Nc3ccc(S(=O)(=O)c4cccc(C[C@H]5C[C@@H](c6ccc7c(c6)CN([C@@H]6CCC(=O)NC6=O)C7=O)C5)c4)cc3F)nc12] | 79 |
| 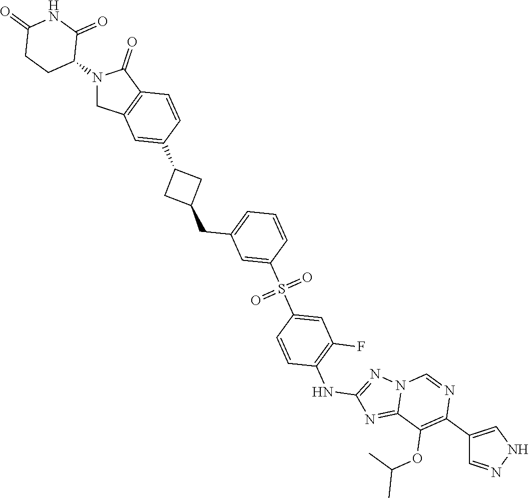 [SMILES: CC(C)Oc1c(-c2cn[nH]c2)ncn2nc(Nc3ccc(S(=O)(=O)c4cccc(C[C@H]5C[C@H](c6ccc7c(c6)CN([C@@H]6CCC(=O)NC6=O)C7=O)C5)c4)cc3F)nc12] | 80 |
| 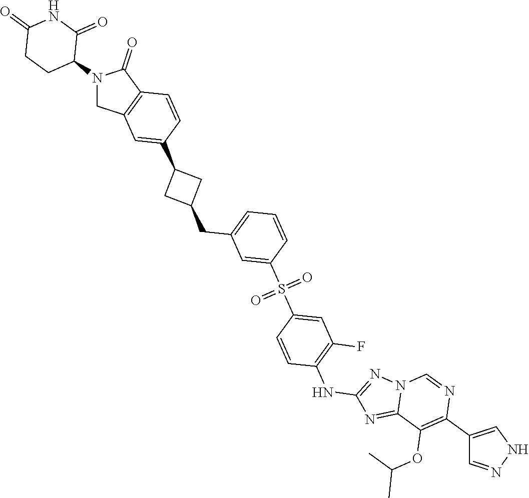 [SMILES: CC(C)Oc1c(-c2cn[nH]c2)ncn2nc(Nc3ccc(S(=O)(=O)c4cccc(C[C@H]5C[C@@H](c6ccc7c(c6)CN([C@H]6CCC(=O)NC6=O)C7=O)C5)c4)cc3F)nc12] | 81 |
| 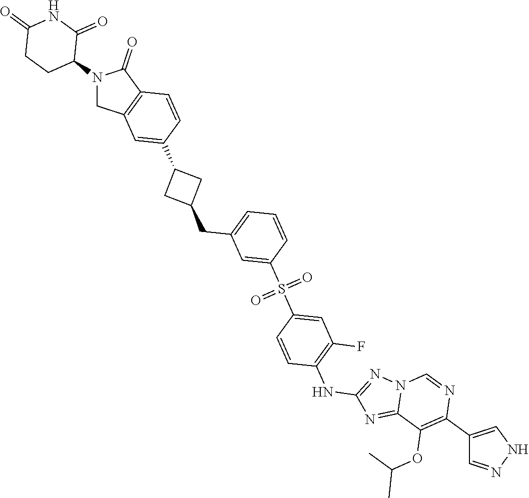 [SMILES: CC(C)Oc1c(-c2cn[nH]c2)ncn2nc(Nc3ccc(S(=O)(=O)c4cccc(C[C@H]5C[C@H](c6ccc7c(c6)CN([C@H]6CCC(=O)NC6=O)C7=O)C5)c4)cc3F)nc12] | 82 |
| 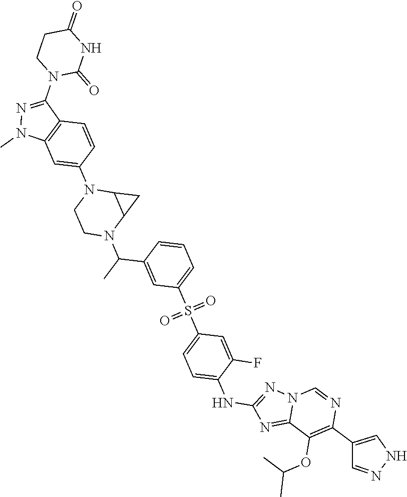 [SMILES: CC(C)Oc1c(-c2cn[nH]c2)ncn2nc(Nc3ccc(S(=O)(=O)c4cccc(C(C)N5CCN(c6ccc7c(N8CCC(=O)NC8=O)nn(C)c7c6)C6CC65)c4)cc3F)nc12] | 83 |
| 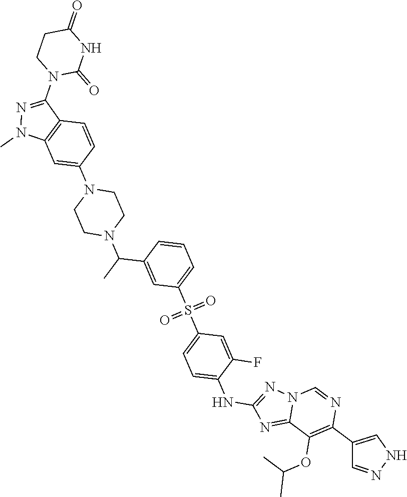 [SMILES: CC(C)Oc1c(-c2cn[nH]c2)ncn2nc(Nc3ccc(S(=O)(=O)c4cccc(C(C)N5CCN(c6ccc7c(N8CCC(=O)NC8=O)nn(C)c7c6)CC5)c4)cc3F)nc12] | 84 |
| 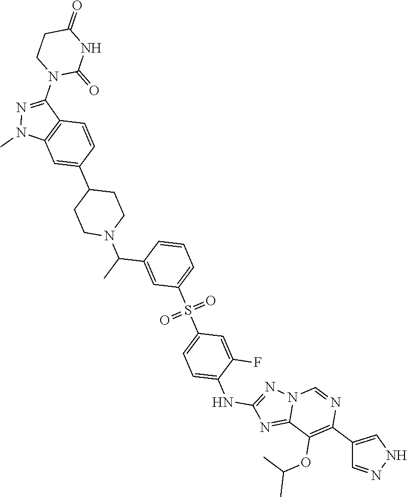 [SMILES: CC(C)Oc1c(-c2cn[nH]c2)ncn2nc(Nc3ccc(S(=O)(=O)c4cccc(C(C)N5CCC(c6ccc7c(N8CCC(=O)NC8=O)nn(C)c7c6)CC5)c4)cc3F)nc12] | 85 |
| 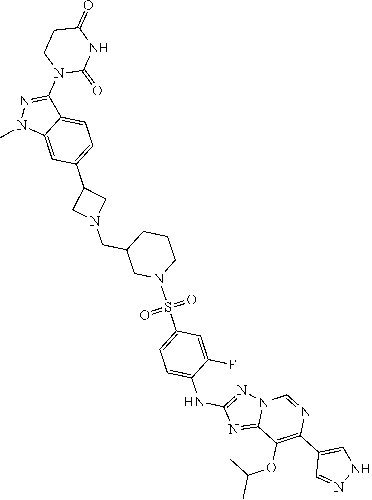 [SMILES: CC(C)Oc1c(-c2cn[nH]c2)ncn2nc(Nc3ccc(S(=O)(=O)N4CCCC(CN5CC(c6ccc7c(N8CCC(=O)NC8=O)nn(C)c7c6)C5)C4)cc3F)nc12] | 86 |
| 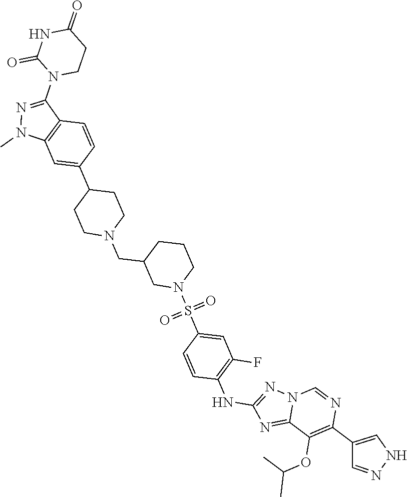 [SMILES: CC(C)Oc1c(-c2cn[nH]c2)ncn2nc(Nc3ccc(S(=O)(=O)N4CCCC(CN5CCC(c6ccc7c(N8CCC(=O)NC8=O)nn(C)c7c6)CC5)C4)cc3F)nc12] | 87 |
| 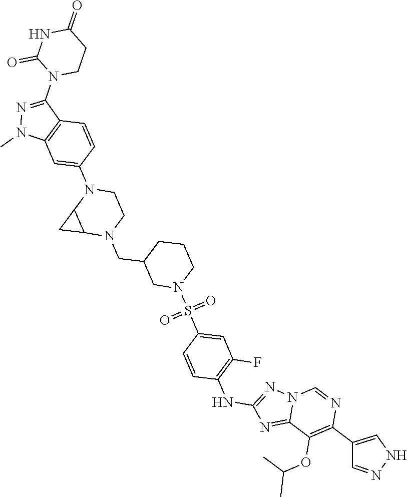 [SMILES: CC(C)Oc1c(-c2cn[nH]c2)ncn2nc(Nc3ccc(S(=O)(=O)N4CCCC(CN5CCN(c6ccc7c(N8CCC(=O)NC8=O)nn(C)c7c6)C6CC65)C4)cc3F)nc12] | 88 |
| 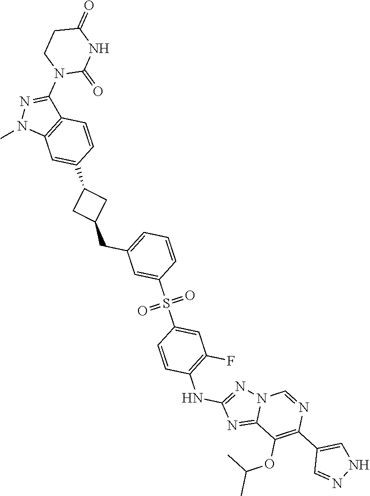 [SMILES: CC(C)Oc1c(-c2cn[nH]c2)ncn2nc(Nc3ccc(S(=O)(=O)c4cccc(C[C@H]5C[C@H](c6ccc7c(N8CCC(=O)NC8=O)nn(C)c7c6)C5)c4)cc3F)nc12] | 89 |
| 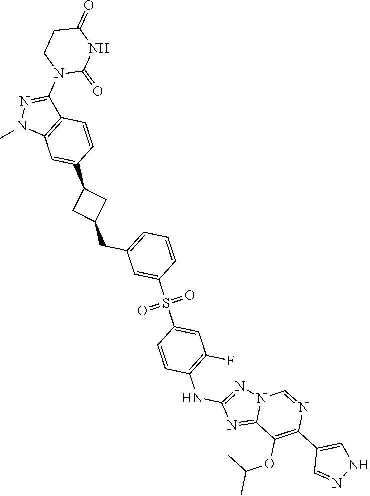 [SMILES: CC(C)Oc1c(-c2cn[nH]c2)ncn2nc(Nc3ccc(S(=O)(=O)c4cccc(C[C@H]5C[C@@H](c6ccc7c(N8CCC(=O)NC8=O)nn(C)c7c6)C5)c4)cc3F)nc12] | 90 |
| 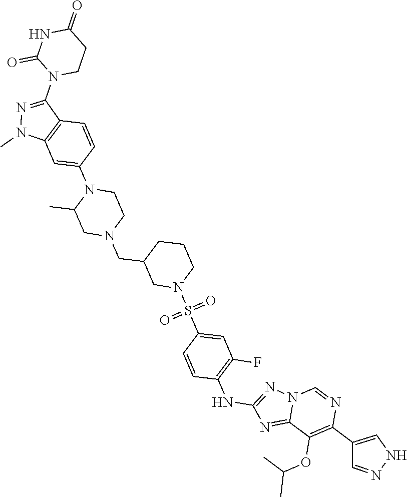 [SMILES: CC(C)Oc1c(-c2cn[nH]c2)ncn2nc(Nc3ccc(S(=O)(=O)N4CCCC(CN5CCN(c6ccc7c(N8CCC(=O)NC8=O)nn(C)c7c6)C(C)C5)C4)cc3F)nc12] | 91 |
| 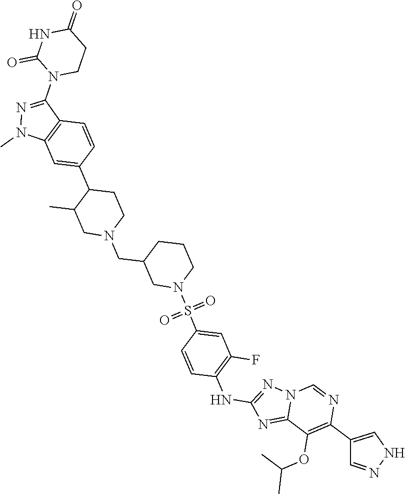 [SMILES: CC(C)Oc1c(-c2cn[nH]c2)ncn2nc(Nc3ccc(S(=O)(=O)N4CCCC(CN5CCC(c6ccc7c(N8CCC(=O)NC8=O)nn(C)c7c6)C(C)C5)C4)cc3F)nc12] | 92 |
| 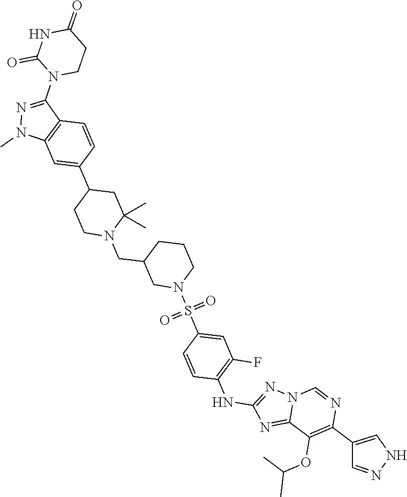 [SMILES: CC(C)Oc1c(-c2cn[nH]c2)ncn2nc(Nc3ccc(S(=O)(=O)N4CCCC(CN5CCC(c6ccc7c(N8CCC(=O)NC8=O)nn(C)c7c6)CC5(C)C)C4)cc3F)nc12] | 93 |
| 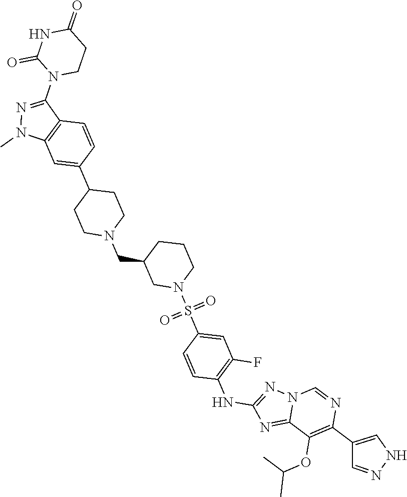 [SMILES: CC(C)Oc1c(-c2cn[nH]c2)ncn2nc(Nc3ccc(S(=O)(=O)N4CCC[C@H](CN5CCC(c6ccc7c(N8CCC(=O)NC8=O)nn(C)c7c6)CC5)C4)cc3F)nc12] | 94 |
| 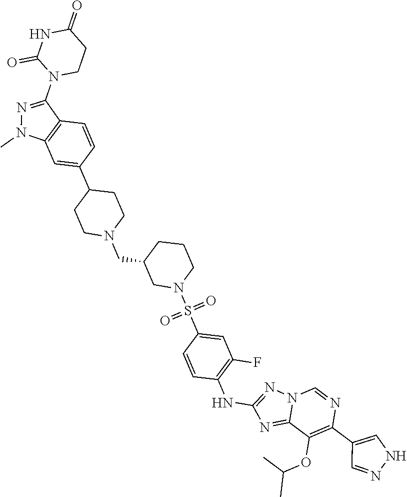 [SMILES: CC(C)Oc1c(-c2cn[nH]c2)ncn2nc(Nc3ccc(S(=O)(=O)N4CCC[C@@H](CN5CCC(c6ccc7c(N8CCC(=O)NC8=O)nn(C)c7c6)CC5)C4)cc3F)nc12] | 95 |
| 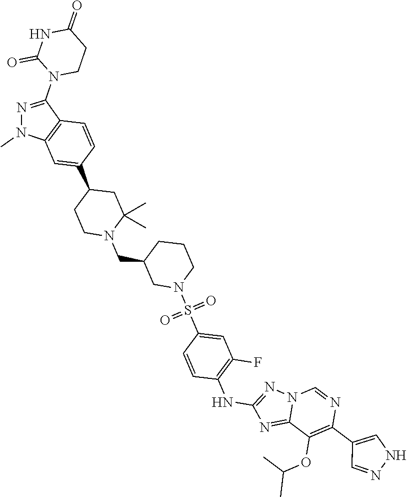 [SMILES: CC(C)Oc1c(-c2cn[nH]c2)ncn2nc(Nc3ccc(S(=O)(=O)N4CCC[C@H](CN5CC[C@@H](c6ccc7c(N8CCC(=O)NC8=O)nn(C)c7c6)CC5(C)C)C4)cc3F)nc12] | 96 |
| 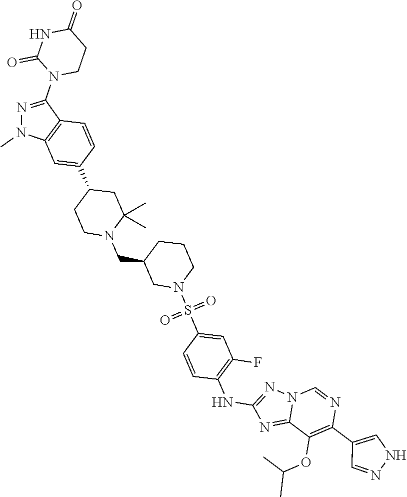 [SMILES: CC(C)Oc1c(-c2cn[nH]c2)ncn2nc(Nc3ccc(S(=O)(=O)N4CCC[C@H](CN5CC[C@H](c6ccc7c(N8CCC(=O)NC8=O)nn(C)c7c6)CC5(C)C)C4)cc3F)nc12] | 97 |
| 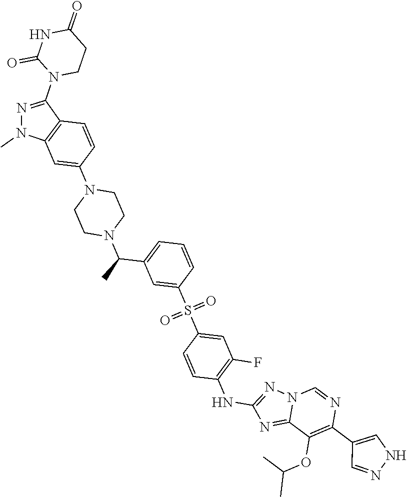 [SMILES: CC(C)Oc1c(-c2cn[nH]c2)ncn2nc(Nc3ccc(S(=O)(=O)c4cccc([C@@H](C)N5CCN(c6ccc7c(N8CCC(=O)NC8=O)nn(C)c7c6)CC5)c4)cc3F)nc12] | 98 |
| 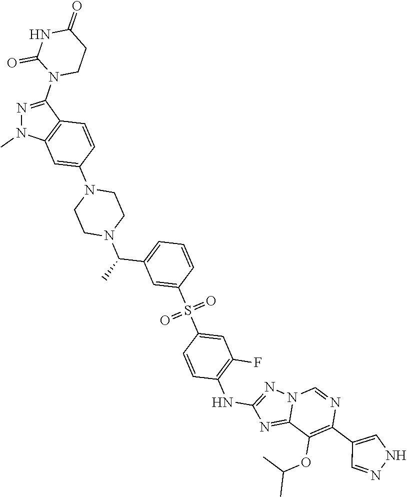 [SMILES: CC(C)Oc1c(-c2cn[nH]c2)ncn2nc(Nc3ccc(S(=O)(=O)c4cccc([C@H](C)N5CCN(c6ccc7c(N8CCC(=O)NC8=O)nn(C)c7c6)CC5)c4)cc3F)nc12] | 99 |
| 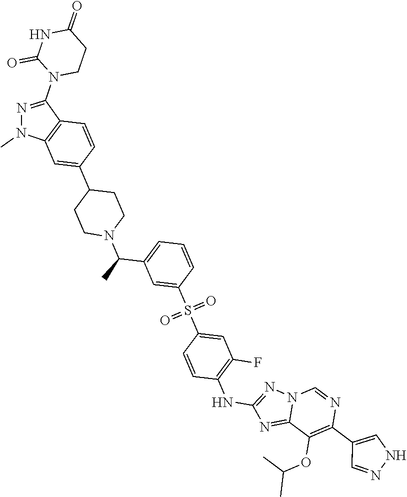 [SMILES: CC(C)Oc1c(-c2cn[nH]c2)ncn2nc(Nc3ccc(S(=O)(=O)c4cccc([C@@H](C)N5CCC(c6ccc7c(N8CCC(=O)NC8=O)nn(C)c7c6)CC5)c4)cc3F)nc12] | 100 |
| 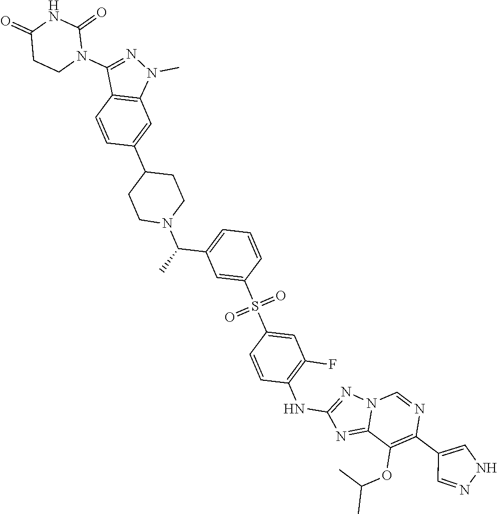 [SMILES: CC(C)Oc1c(-c2cn[nH]c2)ncn2nc(Nc3ccc(S(=O)(=O)c4cccc([C@H](C)N5CCC(c6ccc7c(N8CCC(=O)NC8=O)nn(C)c7c6)CC5)c4)cc3F)nc12] | 101 |
| 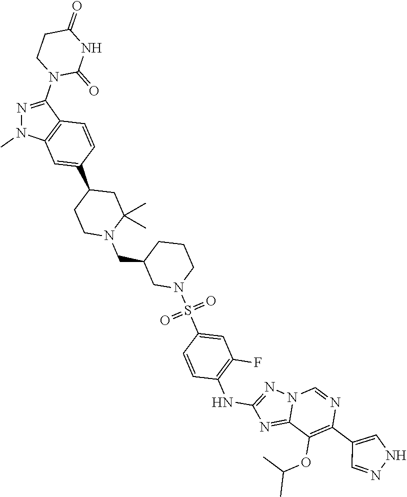 [SMILES: CC(C)Oc1c(-c2cn[nH]c2)ncn2nc(Nc3ccc(S(=O)(=O)N4CCC[C@H](CN5CC[C@@H](c6ccc7c(N8CCC(=O)NC8=O)nn(C)c7c6)CC5(C)C)C4)cc3F)nc12] | 102 |
| 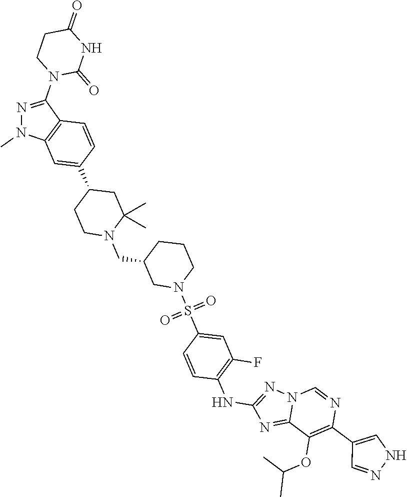 [SMILES: CC(C)Oc1c(-c2cn[nH]c2)ncn2nc(Nc3ccc(S(=O)(=O)N4CCC[C@@H](CN5CC[C@H](c6ccc7c(N8CCC(=O)NC8=O)nn(C)c7c6)CC5(C)C)C4)cc3F)nc12] | 103 |
| 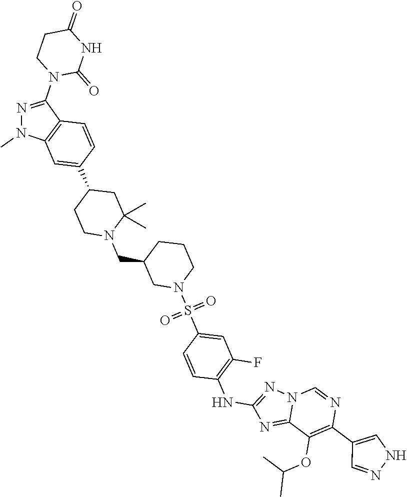 [SMILES: CC(C)Oc1c(-c2cn[nH]c2)ncn2nc(Nc3ccc(S(=O)(=O)N4CCC[C@H](CN5CC[C@H](c6ccc7c(N8CCC(=O)NC8=O)nn(C)c7c6)CC5(C)C)C4)cc3F)nc12] | 104 |
| 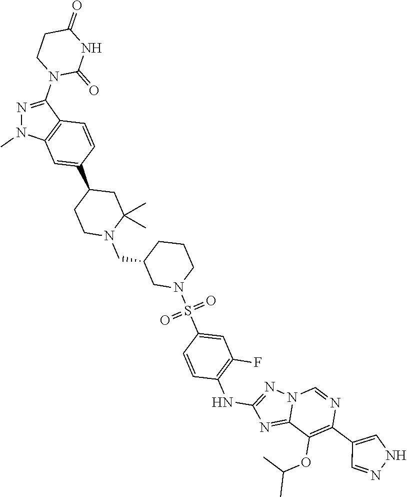 [SMILES: CC(C)Oc1c(-c2cn[nH]c2)ncn2nc(Nc3ccc(S(=O)(=O)N4CCC[C@@H](CN5CC[C@@H](c6ccc7c(N8CCC(=O)NC8=O)nn(C)c7c6)CC5(C)C)C4)cc3F)nc12] | 105 |
| 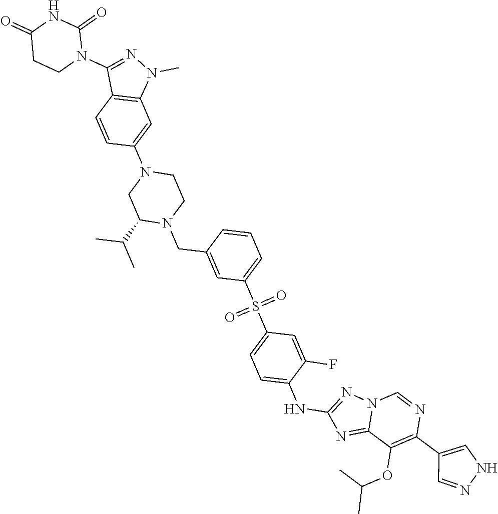 [SMILES: CC(C)Oc1c(-c2cn[nH]c2)ncn2nc(Nc3ccc(S(=O)(=O)c4cccc(CN5CCN(c6ccc7c(N8CCC(=O)NC8=O)nn(C)c7c6)C[C@H]5C(C)C)c4)cc3F)nc12] | 106 |
| 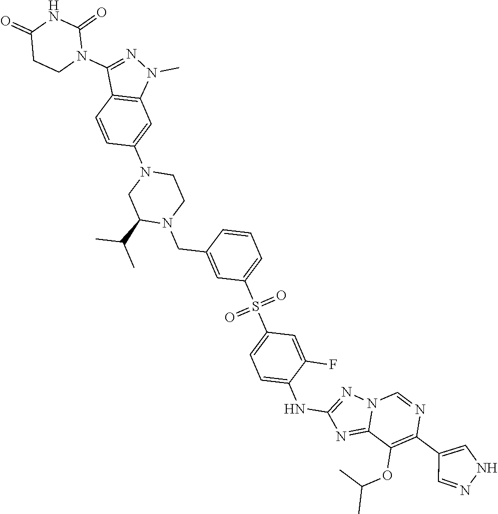 [SMILES: CC(C)Oc1c(-c2cn[nH]c2)ncn2nc(Nc3ccc(S(=O)(=O)c4cccc(CN5CCN(c6ccc7c(N8CCC(=O)NC8=O)nn(C)c7c6)C[C@@H]5C(C)C)c4)cc3F)nc12] | 107 |
| 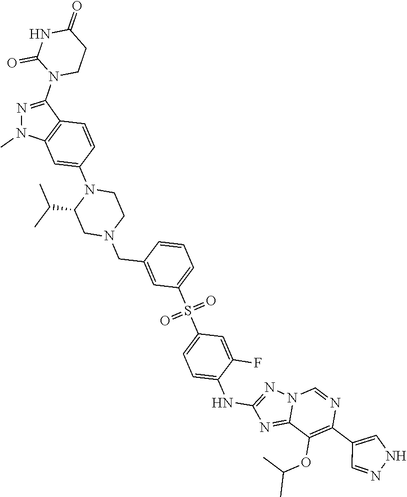 [SMILES: CC(C)Oc1c(-c2cn[nH]c2)ncn2nc(Nc3ccc(S(=O)(=O)c4cccc(CN5CCN(c6ccc7c(N8CCC(=O)NC8=O)nn(C)c7c6)[C@@H](C(C)C)C5)c4)cc3F)nc12] | 108 |
| 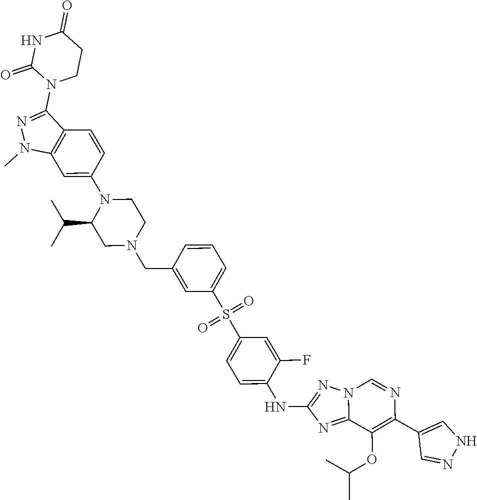 [SMILES: CC(C)Oc1c(-c2cn[nH]c2)ncn2nc(Nc3ccc(S(=O)(=O)c4cccc(CN5CCN(c6ccc7c(N8CCC(=O)NC8=O)nn(C)c7c6)[C@H](C(C)C)C5)c4)cc3F)nc12] | 109 |

Exemplary compounds

| | | CDK2 | CDK2 | CDK9 | CDK9 | CCNE1 | CCNE1 |
|---|---|---|---|---|---|---|---|
| | | HIBIT | HIBIT | HIBIT | HIBIT | HIBIT | HIBIT |
| | | PQ | PQ | PQ | PQ | PQ | PQ |
| | Stereo- | DC50 | Max | DC50 | Max | DC50 | Max |
| Nr | chemistry | (nM) | (%) | (nM) | (%) | (nM) | (%) |
| 1 | | 1.62 | 93 | 2.98 | 104 | 1.94 | 92 |
| 2 | | | 46 | 25.70 | 92 | | 41 |
| 3 | | 3.12 | 66 | 19.40 | 102 | 8.88 | 64 |
| 4 | | 4.14 | 63 | | 4 | 7.10 | 65 |
| 5 | | 1.67 | 92 | 3.91 | 104 | 3.16 | 94 |
| 6 | | 0.59 | 80 | 1.48 | 103 | 0.88 | 73 |
| 7 | | 1.07 | 92 | 2.13 | 91 | 0.76 | 96 |
| 8 | | 1.58 | 92 | 10.20 | 91 | 7.36 | 81 |
| 9 | | 1.44 | 89 | 11.70 | 97 | 6.38 | 72 |
| 10 | | 3.76 | 86 | 25.50 | 54 | 10.60 | 60 |
| 11 | | 2.25 | 90 | | 50 | 14.40 | 65 |
| 12 | | 2.20 | 89 | | 8 | 4.06 | 62 |
| 13 | | 0.42 | 85 | 6.60 | 53 | | 44 |
| 14 | | 0.24 | 70 | 1.12 | 74 | | 33 |
| 15 | | 0.35 | 95 | 1.37 | 99 | 0.72 | 93 |
| 16 | | <0.152 | 91 | 0.52 | 94 | 0.21 | 79 |
| 17 | | 0.26 | 91 | 2.70 | 77 | 0.94 | 83 |
| 18 | | 0.60 | 90 | 2.19 | 87 | 0.64 | 82 |
| 19 | | 0.87 | 90 | 3.80 | 77 | 0.77 | 85 |
| 20 | | 0.8 | 82 | 9.15 | 69 | 1.02 | 54 |
| 21 | | 0.7 | 91 | 5.95 | 83 | 4.09 | 56 |
| 22 | | 1.7 | 93 | 16.10 | 97 | 10.90 | 71 |
| 23 | | 1.1 | 87 | 10.60 | 61 | | 42 |
| 24 | | 0.6 | 94 | | 14 | 3.14 | 88 |
| 25 | | 0.2 | 81 | | 41 | | 21 |
| 26 | | 3.8 | 84 | | 13 | 2.40 | 51 |
| 27 | | 6.3 | 66 | | 6 | | 10 |
| 28 | | | 6 | | 2 | | 0 |
| 29 | | 8.0 | 94 | | 12 | 18.70 | 78 |
| 30 | | 7.2 | 96 | 147.00 | 54 | 29.50 | 79 |
| 31 | | 1.7 | 81 | | 9 | | 26 |
| 32 | | 78.1 | 89 | 396.00 | 62 | >3.00E+03 | 62 |
| 33 | | 19.0 | 91 | | 9 | | 47 |
| 34 | | 5.6 | 86 | | 31 | 14.10 | 62 |
| 35 | | 6.5 | 79 | | 17 | | 25 |
| 36 | | 2.9 | 89 | 57.60 | 87 | 22.10 | 63 |
| 37 | | 2.9 | 84 | 43.70 | 51 | | 30 |
| 38 | | 2.3 | 96 | 24.00 | 63 | 5.76 | 55 |
| 39 | | 3.3 | 96 | 58.50 | 60 | 14.90 | 67 |
| 40 | | 3.6 | 96 | 16.70 | 103 | 8.91 | 89 |
| 41 | | 3.9 | 94 | 28.00 | 57 | | 41 |
| 42 | | 14.9 | 95 | 306.00 | 61 | 78.90 | 67 |
| 43 | | 14.5 | 94 | 307.00 | 75 | 96.50 | 76 |
| 44 | | 3.5 | 92 | | 15 | | 39 |
| 45 | | 4.3 | 85 | | −10 | | 33 |
| 46 | | 3.6 | 95 | 92.10 | 71 | | 48 |
| 47 | | 6.2 | 97 | 135.50 | 74 | 55.00 | 72 |
| 48 | | 3.1 | 79 | | 2 | | 21 |
| 49 | | 2.2 | 98 | 11.60 | 104 | 9.43 | 96 |
| 50 | | 2.8 | 97 | 23.50 | 96 | 15.20 | 90 |
| 51 | | 2.4 | 98 | 20.20 | 100 | 9.29 | 92 |
| 52 | | 2.8 | 98 | 38.70 | 93 | 13.80 | 93 |
| 53 | | 0.2 | 87 | 2.64 | 67 | 0.74 | 61 |
| 54 | | 0.1 | 89 | 1.57 | 74 | 0.59 | 82 |
| 55 | | 0.1 | 91 | 1.13 | 83 | 0.51 | 76 |
| 56 | | | 93 | 0.48 | 90 | 0.23 | 82 |
| 57 | | 0.3 | 90 | 2.25 | 82 | 0.62 | 76 |
| 58 | | 0.1 | 94 | 1.51 | 86 | 0.39 | 86 |
| 59 | | 0.1 | 86 | 1.34 | 68 | 0.40 | 66 |
| 60 | | | 93 | 1.52 | 75 | 0.45 | 74 |
| 61 | | 0.3 | 86 | 3.20 | 78 | 1.55 | 72 |
| 62 | | 0.1 | 91 | 1.22 | 75 | 0.92 | 65 |
| 63 | | <0.152 | 92 | 2.07 | 85 | 1.24 | 66 |
| 64 | | 0.2 | 89 | 3.38 | 74 | 1.79 | 65 |
| 65 | | 0.2 | 85 | | 49 | | 39 |
| 66 | | 0.1 | 92 | 1.87 | 69 | 0.93 | 70 |
| 67 | | <0.152 | 92 | 1.52 | 91 | 1.08 | 73 |
| 68 | | 0.1 | 90 | 2.76 | 73 | 1.05 | 67 |
| 69 | | 0.5 | 71 | | 6 | | 18 |
| 70 | | 0.3 | 61 | | 9 | | 20 |
| 71 | | | 78 | | 29 | | 35 |
| 72 | | | 88 | 0.50 | 71 | 0.17 | 64 |
| 73 | (and) | 0.8 | 79 | | 31 | | 27 |
| 74 | | 0.7 | 91 | 7.04 | 88 | 3.57 | 71 |
| 75 | | 4.7 | 85 | 92.50 | 55 | | 28 |
| 76 | (and) | 5.8 | 87 | | 46 | | 40 |
| 77 | | 12.4 | 95 | | 37 | 34.40 | 82 |
| 78 | | 2.0 | 85 | | 12 | | 36 |
| 79 | | 5.9 | 96 | | 26 | 13.10 | 84 |
| 80 | | 3.9 | 98 | | 49 | 20.30 | 85 |
| 81 | | 24.2 | 86 | | 1 | 62.80 | 57 |
| 82 | | 23.4 | 88 | | 27 | 100.00 | 52 |
| 83 | | 7.6 | 89 | | 23 | | 28 |
| 84 | | 2.6 | 95 | 40.60 | 63 | 17.50 | 57 |
| 85 | | 1.5 | 93 | 12.00 | 82 | 9.64 | 80 |
| 86 | | 1.3 | 56 | | 20 | | 38 |
| 87 | | 0.4 | 90 | 3.32 | 72 | 1.40 | 79 |
| 88 | | 6.5 | 61 | | 18 | | 25 |
| 89 | | 43.4 | 70 | | 17 | | 38 |
| 90 | | 14.3 | 91 | | 14 | | 48 |
| 91 | | 3.2 | 84 | 61.50 | 67 | 12.50 | 71 |
| 92 | | 1.7 | 88 | 21.70 | 77 | 4.11 | 82 |
| 93 | | 0.7 | 77 | 5.87 | 56 | 1.22 | 56 |
| 94 | (or) | 0.3 | 92 | 5.20 | 88 | 1.55 | 89 |
| 95 | (or) | 0.5 | 83 | 7.24 | 71 | 1.02 | 67 |
| 96 | Racemic 2 | 1.0 | 87 | 8.05 | 80 | 3.16 | 75 |
| | enantiomer | | | | | | |
| | mix (or) | | | | | | |
| 97 | Racemic 2 | 0.6 | 68 | | 19 | | 41 |
| | enantiomer | | | | | | |
| | mix (or) | | | | | | |
| 98 | (or) | 2.3 | 95 | 34.80 | 78 | 12.70 | 67 |
| 99 | (or) | 3.0 | 94 | 37.30 | 64 | 10.20 | 52 |
| 100 | (or) | 2.9 | 90 | 12.40 | 86 | 14.30 | 80 |
| 101 | (or) | 2.6 | 90 | 10.60 | 82 | 13.90 | 77 |
| 102 | (or) | 0.5 | 90 | 5.65 | 75 | 1.34 | 76 |
| 103 | (or) | 1.0 | 90 | 4.01 | 92 | 1.96 | 83 |
| 104 | (or) | 1.0 | 75 | | 13 | | 48 |
| 105 | (or) | 0.9 | 63 | | 25 | | 24 |
| 106 | (or) | 8.3 | 94 | | | 39.40 | 59 |
| 107 | (or) | 12.0 | 93 | | | 63.10 | 71 |
| 108 | (or) | 10.4 | 92 | | | 79.80 | 77 |
| 109 | (or) | 9.7 | 94 | | | 71.70 | 78 |

PQ = Protein Quantification;
Max = Maximum

### Alternative Embodiments

In an alternative embodiment, compounds described herein may also comprise one or more isotopic substitutions. For example, hydrogen may be ²H (D or deuterium) or ³H (T or tritium); carbon may be, for example, ¹³C or ¹⁴C; oxygen may be, for example, 180; nitrogen may be, for example, ¹⁵N, and the like. In other embodiments, a particular isotope (e.g., ³H, ¹³C, ¹⁴C, ¹⁸O, or ¹⁵N) can represent at least 1%, at least 5%, at least 10%, at least 15%, at least 20%, at least 25%, at least 30%, at least 35%, at least 40%, at least 45%, at least 50%, at least 60%, at least 65%, at least 70%, at least 75%, at least 80%, at least 85%, at least 90%, at least 95%, at least 99%, or at least 99.9% of the total isotopic abundance of an element that occupies a specific site of the compound.

### Pharmaceutical Compositions

In an embodiment, provided is a pharmaceutical composition comprising a pharmaceutically acceptable carrier and an effective amount of a compound described herein (e.g., a compound of Formula A-I or Formula I or a compound of Table 1), or a pharmaceutically acceptable salt thereof.

The term “pharmaceutically acceptable carrier or adjuvant” refers to a carrier or adjuvant that may be administered to a patient, together with a compound provided herewith, and which does not destroy the pharmacological activity thereof and is nontoxic when administered in doses sufficient to deliver a therapeutic amount of the compound.

Pharmaceutically acceptable carriers, adjuvants and vehicles that may be used in the pharmaceutical compositions provided herewith include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose based substances, polyethylene glycol, sodium carboxymethylcellulose, polyacrylates, waxes, polyethylene polyoxypropylene block polymers, polyethylene glycol and wool fat. Cyclodextrins such as α-, β-, and γ-cyclodextrin, or chemically modified derivatives such as hydroxyalkylcyclodextrins, including 2 and 3 hydroxypropyl-o-cyclodextrins, or other solubilized derivatives may also be advantageously used to enhance delivery of compounds of the formulae described herein.

When employed as pharmaceuticals, the compounds provided herein are typically administered in the form of a pharmaceutical composition. Such compositions can be prepared in a manner well known in the pharmaceutical art and comprise at least one active compound.

In an embodiment, with respect to the pharmaceutical composition, the carrier is a parenteral carrier, oral or topical carrier.

Also provided is a compound described herein (e.g., a compound of Formula A-I or Formula I or a compound of Table 1, or pharmaceutically acceptable salts thereof) (or pharmaceutical composition thereof) for use as a pharmaceutical or a medicament (e.g., a medicament for the treatment of a CDK2 or CCNE (CCNE1 and/or CCNE2)-mediated disease or disorder in a subject in need thereof). In an embodiment, the disease is a CDK2 mediated disease. In an embodiment, the disease is a CCNE (CCNE1 and/or CCNE2)-mediated disease. In an embodiment, the disease is a CDK2 and a CCNE (CCNE1 and/or CCNE2)-mediated disease. In an embodiment, the disease or disorder is a proliferating disease or disorder. In a further embodiment, the disease or disorder is a cancer. In an embodiment, the cancer is selected from ovarian cancer, gastric cancer, uterine cancer (e.g., endometrial cancer), and breast cancer (e.g., triple negative breast cancer (TNBC), hormone-receptor positive (HR+) breast cancer, HER2 positive (HER2+) positive breast cancer).

Also provided is a compound described herein (e.g., a compound of Formula A-I or Formula I or a compound of Table 1, or pharmaceutically acceptable salts thereof) (or pharmaceutical composition thereof) for use in the treatment of a CDK2 or CCNE (CCNE1 and/or CCNE2)-mediated disease or disorder in a subject in need thereof. In an embodiment, the disease is a CDK2 mediated disease. In an embodiment, the disease is a CCNE (CCNE1 and/or CCNE2)-mediated disease. In an embodiment, the disease is a CDK2 and a CCNE (CCNE1 and/or CCNE2)-mediated disease. In an embodiment, the disease or disorder is a proliferating disease or disorder. In a further embodiment, the disease or disorder is a cancer. In an embodiment, the cancer is selected from ovarian cancer, gastric cancer, uterine cancer (e.g., endometrial cancer), and breast cancer (e.g., triple negative breast cancer (TNBC), hormone-receptor positive (HR+) breast cancer, HER2 positive (HER2+) positive breast cancer).

Also provided is a compound described herein (e.g., a compound of Formula A-I or Formula I or a compound of Table 1, or pharmaceutically acceptable salts thereof) (or pharmaceutical composition thereof) for use in the manufacturing of a medicament (e.g., a medicament for the treatment of a CDK2 or CCNE (CCNE1 and/or CCNE2)-mediated disease or disorder in a subject in need thereof). In an embodiment, the disease or disorder is a proliferating disease or disorder. In an embodiment, the disease is a CDK2 mediated disease. In an embodiment, the disease is a CCNE (CCNE1 and/or CCNE2)-mediated disease. In an embodiment, the disease is a CDK2 and a CCNE (CCNE1 and/or CCNE2)-mediated disease. In a further embodiment, the disease or disorder is a cancer. In an embodiment, the cancer is selected from ovarian cancer, gastric cancer, uterine cancer (e.g., endometrial cancer), and breast cancer (e.g., triple negative breast cancer (TNBC), hormone-receptor positive (HR+) breast cancer, HER2 positive (HER2+) positive breast cancer).

Generally, the compounds provided herein are administered in an effective amount (e.g., a therapeutically effective amount). The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

The pharmaceutical compositions provided herewith may be administered orally, parenterally, by inhalation spray, topically, rectally, nasally, buccally, vaginally or via an implanted reservoir, preferably by oral administration or administration by injection. The pharmaceutical compositions provided herewith may contain any conventional nontoxic pharmaceutically acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrastemal, intrathecal, intralesional and intracranial injection or infusion techniques.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable carriers known in the art. As before, the active compound in such compositions is typically a minor component, often being from about 0.05 to 10% by weight with the remainder being the injectable carrier and the like. The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

Transdermal compositions are typically formulated as a topical ointment or cream containing the active ingredient(s), generally in an amount ranging from about 0.01 to about 20% by weight, preferably from about 0.1 to about 20% by weight, preferably from about 0.1 to about 10% by weight, and more preferably from about 0.5 to about 15% by weight. When formulated as an ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or the formulation. All such known transdermal formulations and ingredients are included within the scope provided herein.

The compounds provided herein can also be administered by a transdermal device. Accordingly, transdermal administration can be accomplished using a patch either of the reservoir or porous membrane type, or of a solid matrix variety.

The pharmaceutical compositions provided herewith may also be administered in the form of suppositories for rectal administration. These compositions can be prepared by mixing a compound provided herewith with a suitable nonirritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions provided herewith may be administered by nasal aerosol or inhalation. Such compositions are prepared according to techniques well known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

The above-described components for orally administrable, injectable or topically administrable, rectally administrable and nasally administrable compositions are merely representative. Other materials as well as processing techniques and the like are set forth in Part 8 of Remington's Pharmaceutical Sciences, 17th edition, 1985, Mack Publishing Company, Easton, Pennsylvania, which is incorporated herein by reference.

The compounds described herein can also be administered in sustained release forms or from sustained release drug delivery systems. A description of representative sustained release materials can be found in Remington's Pharmaceutical Sciences.

When the compositions provided herewith comprise a combination of a compound of the formulae described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds provided herewith. Alternatively, those agents may be part of a single dosage form, mixed together with the compounds provided herewith in a single composition.

Also provided is the pharmaceutically acceptable acid addition salt of a compound described herein (e.g., compound of Formula A-I or Formula I or a compound of Table 1).

The acid which may be used to prepare the pharmaceutically acceptable salt is that which forms a non-toxic acid addition salt, i.e., a salt containing pharmacologically acceptable anions such as the hydrochloride, hydroiodide, hydrobromide, nitrate, sulfate, bisulfate, phosphate, acetate, lactate, citrate, tartrate, succinate, maleate, fumarate, benzoate, para-toluenesulfonate, and the like.

The compounds described herein can, for example, be administered by injection, intravenously, intraarterially, subdermally, intraperitoneally, intramuscularly, or subcutaneously; or orally, buccally, nasally, transmucosally, topically, in an ophthalmic preparation, or by inhalation, with a dosage ranging from about 0.5 to about 100 mg/kg of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect. Typically, the pharmaceutical compositions provided herewith will be administered from about 1 to about 6 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination provided herewith may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long term basis upon any recurrence of disease symptoms.

### Methods of Treatment

Compounds of the present disclosure can inhibit CDK2 and/or CCNE (CCNE1 and/or CCNE2) and therefore are useful for treating diseases wherein the underlying pathology is, wholly or partially, mediated by CDK2 and/or CCNE (CCNE1 and/or CCNE2). In an embodiment, the disease pathology is wholly or partially, mediated by CDK2. In an embodiment, the disease pathology is wholly or partially, mediated by CCNE (CCNE1 and/or CCNE2). In an embodiment, the disease pathology is wholly or partially, mediated by CDK2 and CCNE (CCNE1 and/or CCNE2). Such diseases include cancer and other diseases with proliferation disorder.

In an embodiment, the compounds of Formula A-I or Formula I inhibit both CDK2 and CCNE (CCNE1 and/or CCNE2). In an embodiment the compounds of Formula A-I or Formula I inhibit CDK2 (e.g., selectively inhibit CDK2 over CCNE (CCNE1 and/or CCNE2)). In an embodiment the compounds of Formula A-I or Formula I inhibit CCNE (CCNE1 and/or CCNE2) (e.g., selectively inhibit CCNE (CCNE1 and/or CCNE2) over CDK2).

In an embodiment, the present disclosure provides treatment of an individual or a patient in vivo using a compound of Formula A-I or Formula I or a salt thereof such that growth of cancerous tumors is inhibited. A compound of Formula A-I or Formula I or of any of the formulas as described herein, or a compound as recited in any of the claims and described herein, or a salt thereof, can be used to inhibit the growth of cancerous tumors with aberrations that activate the CDK2 kinase activity. These include, but are not limited to, disease (e.g., cancers) that are characterized by amplification or overexpression of CCNE (CCNE1 and/or CCNE2) such as ovarian cancer, uterine carcinosarcoma and breast cancer and p27 inactivation such as breast cancer and melanomas. Accordingly, in an embodiment of the methods, the patient has been previously determined to have an amplification of the cyclin E (CCNE (CCNE1 and/or CCNE2)) gene and/or an expression level of CCNE (CCNE1 and/or CCNE2) in a biological sample obtained from the human subject that is higher than a control expression level of CCNE (CCNE1 and/or CCNE2). In some embodiments, the cancers are characterized by amplification or overexpression of CCNE1. Accordingly, in an embodiment of the methods, the patient has been previously determined to have an amplification of the cyclin E1 gene and/or an expression level of CCNE1 in a biological sample obtained from the human subject that is higher than a control expression level of CCNE1. Alternatively, a compound of Formula A-I or Formula I or of any of the formulas as described herein, or a compound as recited in any of the claims and described herein, or a salt thereof, can be used in conjunction with other agents or standard cancer treatments.

In an embodiment, the present disclosure provides a method for inhibiting growth of tumor cells in vitro. The method includes contacting the tumor cells in vitro with a compound of Formula A-I or Formula I or of any of the formulas as described herein, or of a compound as recited in any of the claims and described herein, or of a salt thereof.

In an embodiment, the present disclosure provides a method for inhibiting growth of tumor cells with CCNE (CCNE1 and/or CCNE2) amplification and overexpression in an individual or a patient. The method includes administering to the individual or patient in need thereof a therapeutically effective amount of a compound of Formula A-I or Formula I or of any of the formulas as described herein, or of a compound as recited in any of the claims and described herein, or a salt or a stereoisomer thereof.

In an embodiment, provided herein is a method of inhibiting and/or degrading CDK2 and/or CCNE (CCNE1 and/or CCNE2), comprising contacting the CDK2 and/or CCNE (CCNE1 and/or CCNE2) with a compound of Formula A-I or Formula I or any of the formulas as described herein, a compound as recited in any of the claims and described herein, or a salt thereof. In an embodiment, provided herein is a method of inhibiting and/or degrading CDK2, comprising contacting the CDK2 with a compound of Formula A-I or Formula I or any of the formulas as described herein, a compound as recited in any of the claims and described herein, or a salt thereof. In an embodiment, provided herein is a method of inhibiting and/or degrading CCNE (CCNE1 and/or CCNE2), comprising contacting the CCNE (CCNE1 and/or CCNE2) with a compound of Formula A-I or Formula I or any of the formulas as described herein, a compound as recited in any of the claims and described herein, or a salt thereof. In an embodiment, provided herein is a method of inhibiting and/or degrading CDK2 and CCNE (CCNE1 and/or CCNE2), comprising contacting the CDK2 and CCNE (CCNE1 and/or CCNE2) with a compound of Formula A-I or Formula I or any of the formulas as described herein, a compound as recited in any of the claims and described herein, or a salt thereof.

In an embodiment, provided herein is a method of inhibiting and/or degrading CDK2 and/or CCNE (CCNE1 and/or CCNE2) in a patient, comprising administering to the patient a compound of Formula A-I or Formula I or any of the formulas as described herein, a compound as recited in any of the claims and described herein, or a salt thereof.

In an embodiment, provided herein is a method for treating cancer. The method includes administering to a patient (in need thereof), a therapeutically effective amount of a compound of Formula A-I or Formula I or any of the formulas as described herein, a compound as recited in any of the claims and described herein, or a salt thereof.

In an embodiment, the cancer is characterized by amplification or overexpression of CCNE (CCNE1 and/or CCNE2). In an embodiment, the cancer is ovarian cancer or breast cancer, characterized by amplification or overexpression of CCNE (CCNE1 and/or CCNE2).

In an embodiment, provided herein is a method of treating a disease or disorder associated with CDK2 and/or CCNE (CCNE1 and/or CCNE2) in a patient, comprising administering to the patient a therapeutically effective amount of a compound of Formula A-I or Formula I or any of the formulas as described herein, a compound as recited in any of the claims and described herein, or a salt thereof. In an embodiment, provided herein is a method of treating a disease or disorder associated with CDK2 in a patient, comprising administering to the patient a therapeutically effective amount of a compound of Formula A-I or Formula I or any of the formulas as described herein, a compound as recited in any of the claims and described herein, or a salt thereof. In an embodiment, provided herein is a method of treating a disease or disorder associated with CCNE (CCNE1 and/or CCNE2) in a patient, comprising administering to the patient a therapeutically effective amount of a compound of Formula A-I or Formula I or any of the formulas as described herein, a compound as recited in any of the claims and described herein, or a salt thereof. In an embodiment, provided herein is a method of treating a disease or disorder associated with CDK2 and CCNE (CCNE1 and/or CCNE2) in a patient, comprising administering to the patient a therapeutically effective amount of a compound of Formula A-I or Formula I or any of the formulas as described herein, a compound as recited in any of the claims and described herein, or a salt thereof.

In an embodiment, the disease or disorder associated with CDK2 and/or CCNE (CCNE1 and/or CCNE2) is associated with an amplification of the cyclin E1 (CCNE1) gene and/or overexpression of CCNE1. In an embodiment, the disease or disorder associated with CDK2 and/or CCNE (CCNE1 and/or CCNE2) is N-myc amplified neuroblastoma cells (see Molenaar, et al., Proc Natl Acad Sci USA 106(31): 12968-12973) K-Ras mutant lung cancers (see Hu, S., et al., Mol Cancer Ther, 2015. 14(11): 2576-85, and cancers with FBW7 mutation and CCNE (CCNE1 and/or CCNE2) overexpression (see Takada, et al., Cancer Res, 2017.77(18): 4881-4893).

In an embodiment, the disease or disorder associated with CDK2 and/or CCNE (CCNE1 and/or CCNE2) is lung squamous cell carcinoma, lung adenocarcinoma, pancreatic adenocarcinoma, breast invasive carcinoma, uterine carcinosarcoma, ovarian serous cystadenocarcinoma, stomach adenocarcinoma, esophageal carcinoma, bladder urothelial carcinoma, mesothelioma, or sarcoma.

In an embodiment, the disease or disorder associated with CDK2 and/or CCNE (CCNE1 and/or CCNE2) is lung adenocarcinoma, breast invasive carcinoma, uterine carcinosarcoma, ovarian serous cystadenocarcinoma, or stomach adenocarcinoma.

In an embodiment, the disease or disorder associated with CDK2 and/or CCNE (CCNE1 and/or CCNE2) is an adenocarcinoma, carcinoma, or cystadenocarcinoma. In an embodiment, the disease or disorder associated with CDK2 and/or CCNE (CCNE1 and/or CCNE2) is uterine cancer, ovarian cancer, stomach cancer, esophageal cancer, lung cancer, bladder cancer, pancreatic cancer, or breast cancer.

In an embodiment, the disease or disorder associated with CDK2 and/or CCNE (CCNE1 and/or CCNE2) is a cancer.

In an embodiment, the cancer is characterized by amplification or overexpression of CCNE (CCNE1 and/or CCNE2). In an embodiment, the cancer is ovarian cancer or breast cancer, characterized by amplification or overexpression of CCNE (CCNE1 and/or CCNE2).

In an embodiment of any of the embodiments described herein, the CCNE is CCNE1. In an embodiment, the CCNE is CCNE2. In an embodiment, the CCNE is CCNE1 and CCNE2.

In and embodiment, the cancer has primary or acquired resistance to CDK4/6 inhibition (e.g., is resistant to treatment with CDK4/6 inhibitors).

In an embodiment, the breast cancer is chemotherapy or radiotherapy resistant breast cancer, endocrine resistant breast cancer, trastuzumab resistant breast cancer, or breast cancer demonstrating primary or acquired resistance to CDK4/6 inhibition. In an embodiment, the breast cancer is advanced or metastatic breast cancer.

Additionally, the disclosure includes refractory or recurrent malignancies whose growth may be inhibited using the compounds of the disclosure.

In an embodiment, the compounds of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

### Selected Embodiments

Embodiment 1. A compound of Formula A-I:

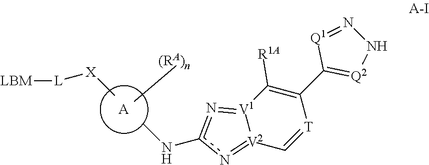

or a pharmaceutically acceptable salt thereof, wherein:
[symbol] is a single or a double bond;
Ring A is selected from the group consisting of a nitrogen-containing 4-10 member heterocyclyl, a C₆₋₁₀ aryl and a 5-10-member heteroaryl, wherein the 4-10 member heterocyclyl, C₆₋₁₀ aryl and 5-10-member heteroaryl are attached to the —NH— through a carbon atom;
V¹ is nitrogen and V² is carbon, and Ring

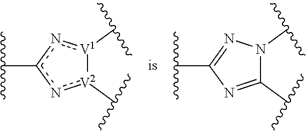

or V² is nitrogen and V¹ is carbon, and Ring

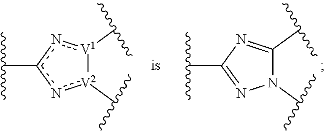

T is CH or N;
Q¹ and Q² are independently selected from N and CH;
R^{1A} is independently selected from H, D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocyclyl, 5-10 membered heteroaryl, C₃₋₁₀ cycloalkyl-C₁₋₄ alkyl, 6-10 membered aryl-C₁₋₄ alkyl-, 4-10 membered heterocyclyl-C₁₋₄ alkyl-, 5-10 membered heteroaryl-C₁₋₄ alkyl-, OR^{a1}, SR^{a1} NHOR^{a1}, C(O)R^{b1}, C(O)NR^{a1}R^{a1}, C(O)NR^{a1}(OR^{a1}), C(O)OR^{a1}, OC(O)R^{b1}, OC(O)NR^{a1}R^{a1}, 10 NR^{a1}R^{a1}, NR^{a1}NR^{a1}R^{a1}, NR^{a1}C(O)R^{b1}, NR^{a1}C(O)OR^{a1}, NR^{a1}C(O)NR^{a1}R^{a1}, C(═NR^{a1})R^{b1}, C(═NR^{a1})NR^{a1}R^{a1}, NR^{a1}C(═NR^{a1})NR^{a1}R^{a1}, NR^{a1}C(═NR^{a1})R^{b1}, NR^{a1}S(O)NR^{a1}R^{a1}, NR^{a1}S(O)R^{a1}, NR^{a1}S(O)₂R^{b1}, NR^{a1}S(O)(═NR^{a1})R^{b1}, NR^{a1}S(O)₂NR^{a1}R^{a1}, S(O)R^{b1}, S(O)NR^{a1}R^{a1}, S(O)₂R^{b1}, S(O)₂NR^{a1}R^{a1}, OS(O)(═NR^{a1})R^{b1}, OS(O)₂R^{b1}, S(O)(═NR^{a1})R^{b1}, SF₅, P(O)R^{a1}R^{b1}, OP(O)(OR^{a1})(OR^{a1}) and P(O)(OR^{a1})(OR^{a1}), wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocyclyl, 5-10 membered heteroaryl, C₃₋₁₀ cycloalkyl-C₁₋₄ alkyl-, 6-10 membered aryl-C₁₋₄ alkyl-, 4-10 membered heterocyclyl-C₁₋₄ alkyl-, and 5-10 membered heteroaryl-C₁₋₄ alkyl- are each substituted with 0, 1, 2, 3, or 4 independently selected R² substituents;
each R² is independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocyclyl, 5-6 membered heteroaryl, C₃₋₇ cycloalkyl-C₁₋₄ alkyl-, phenyl-C₁₋₄ alkyl-, 4-7 membered heterocyclyl-C₁₋₄ alkyl-, 5-6 membered heteroaryl-C₁₋₄ alkyl-, OR^{a2}, SR^{a2}, NHOR^{a2}, C(O)R^{b2}, C(O)NR^{a2}R^{a2}, C(O)NR^{a2}(OR^{a2}), C(O)OR^{a2}, OC(O)R^{b2}, OC(O)NR^{a2}R^{a2}, NR^{a2}R^{a2}, NR^{a2}NR^{a2}R^{a2}, NR^{a2}C(O)R^{b2}, NR^{a2}C(O)OR^{a2}, NR^{a2}C(O)NR^{a2}R^{a2}, C(═NR^{a2})R^{b2}, C(═NR^{a2})NR^{a2}R^{a2}, NR^{a2}C(═NR^{a2})NR^{a2}R^{a2}, NR^{a2}C(═NR^{a2})R^{b2}, NR^{a2}S(O)NR^{a2}R^{a2} NR^{a2}S(O)R^{b2}, NR^{a2}S(O)₂R^{b2}, NR^{a2}S(O)(═NR^{a2})R^{b2}, NR^{a2}S(O)₂NR^{a2}R^{a2}, S(O)R^{b2} S(O)NR^{a2}R^{a2}, S(O)₂R^{b2}, S(O)₂NR^{a2}R^{a2}, OS(O)(═NR^{a2})R^{b2}, OS(O)₂R^{b2}, S(O)(═NR^{a2})R^{b2}, SF₅, P(O)R^{a2}R^{a2}, OP(O)(OR^{a2})(OR^{a2}) and P(O)(OR^{a2})(OR^{a2}), wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocyclyl, 5-6 membered heteroaryl, C₃₋₇ cycloalkyl-C₁₋₄ alkyl-, phenyl-C₁₋₄ alkyl, 4-7 membered heterocyclyl-C₁₋₄ alkyl-, and 5-6 membered heteroaryl-C₁₋₄ alkyl- are each substituted with 0, 1, 2, 3, or 4 substituents independently selected from C₁₋₄ alkyl, C₃₋₇ cycloalkyl, cyclopropyl, oxo, —C(O)C₁₋₄alkyl, —C(O)OC₁₋₄alkyl, —C(O)NH₂, —OH, —F, —Cl, —O—C₁₋₄alkyl and —CN;
each instance of R^{A} is independently selected from —D, halo, CN, C₁₋₄ alkyl, C₁₋₄ haloalkyl, C₃₋₆ cycloalkyl, 4-6 membered heterocyclyl, OR^{a1}, SR^{a1}, SF₅, NR^{a1}R^{a1}, C₃₋₆ cycloalkyl-C₁₋₃ alkyl-, and 4-6 membered heterocyclyl-C₁₋₃ alkyl-, wherein said C₁₋₄ alkyl, C₁₋₄ haloalkyl-, C₃₋₆ cycloalkyl, 4-6 membered heterocyclyl-, C₃₋₆ cycloalkyl-C₁₋₃ alkyl-, and 4-6 membered heterocyclyl-C₁₋₃ alkyl are substituted with 0, 1, 2, 3, or 4 substituents independently selected from —D, halo, —OH, —C₁₋₄ alkyl and —OC₁₋₄ alkyl;
or, alternatively, two R^{A} groups on adjacent atoms of Ring A, together with the ring atoms to which they are attached, form Ring D, wherein Ring D is selected from C₃₋₆ cycloalkyl, 4-6 membered heterocyclyl, phenyl, and 5-6 membered heteroaryl, each of which is substituted with 0, 1, 2, 3, or 4 substituents independently selected from —D, halo, —OH, —C₁₋₄ alkyl and —OC₁₋₄ alkyl;
each R^{a1} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocyclyl, 5-10 membered heteroaryl, C₃₋₁₀ cycloalkyl-C₁₋₄ alkyl-, 6-10 membered aryl-C₁₋₄ alkyl-, 4-10 membered heterocyclyl-C₁₋₄ alkyl-, and 5-10 membered heteroaryl-C₁₋₄ alkyl-, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocyclyl, 5-10 membered heteroaryl, C₃₋₁₀ cycloalkyl-C₁₋₄ alkyl, 6-10 membered aryl-C₁₋₄ alkyl, 4-10 membered heterocyclyl-C₁₋₄ alkyl, and 5-10 membered heteroaryl-C₁₋₄ alkyl are each substituted with 0, 1, 2, 3, or 4 independently selected R² substituents; or two R^{a1} groups attached to the same nitrogen atom together with the nitrogen to which they are attached form a 4-7-membered heterocyclyl group substituted with 0, 1, 2, 3, or 4 substituents independently selected from —D, halo, —OH, —C₁₋₄ alkyl and —OC₁₋₄ alkyl;
each R^{b1} is independently selected from C₁₋₆ alkyl, C₁₋₆ hydroxyalkyl, C₃₋₉ cycloalkyl and C₂₋₆ heteroalkyl;
   each R^{a2} is independently selected from H, C₁₋₆ alkyl, C₁₋₆ hydroxyalkyl, C₃₋₉ cycloalkyl and C₂₋₆ heteroalkyl, or, when possible, two instances of R^{a2} and the atom to which they are attached are taken together to form a 4-7 member heterocycle substituted with 0, 1, 2, 3, or 4 substituents independently selected from —D, halo, —OH, —C₁₋₄ alkyl and —OC₁₋₄ alkyl;
each R^{b2} is independently selected from C₁₋₆ alkyl, C₁₋₆ hydroxyalkyl, C₃₋₉ cycloalkyl and C₂₋₆ heteroalkyl;
X is X¹ when Ring A is heterocyclyl and is selected from X¹ and X² when Ring A is aryl or heteroaryl;
X¹ is selected from —S(O)₂— and —C(O)—;
X² is selected from —O—, —NH—, —N(CH₃)— and —CH₂—;
L is a covalent bond or a bivalent, saturated or unsaturated, straight or branched C₁₋₅₀ hydrocarbon chain, wherein 0-6 methylene units of L are independently replaced by —Cy—, —CH(R)—, —C(R)₂—, —O—, —NR—, —S—, —OC(═O)—, —C(═O)O—, —C(═O)—, —S(═O)—, —S(═O)₂—, —NRS(═O)₂—, —S(═O)₂NR—, —NRC(═O)—, —C(═O)NR—, —OC(═O)NR— or —NRC(═O)O—, wherein:
each —Cy— is independently a bivalent ring selected from phenylene, an 8-10 membered bicyclic arylene, a 4-7 membered monocyclic carbocyclylene, a 5-11 membered spiro carbocyclylene, a 4-10 membered bicyclic carbocyclylene, a 5-10 membered bridged carbocyclylene, a 4-7 membered monocyclic heterocyclylene having 1-2 heteroatoms independently selected from nitrogen, oxygen, and sulfur, a 5-11 membered spiro heterocyclylene having 1-2 heteroatoms independently selected from nitrogen, oxygen, and sulfur, an 4-10 membered bicyclic heterocyclylene having 1-2 heteroatoms independently selected from nitrogen, oxygen, and sulfur, a 5-10 membered bridged bicyclic saturated or partially unsaturated heterocyclylene having 1-2 heteroatoms independently selected from nitrogen, oxygen, and sulfur, a 5-6 membered heteroarylene having 1-4 heteroatoms independently selected from nitrogen, oxygen, and sulfur, or an 8-10 membered bicyclic heteroarylene having 1-5 heteroatoms independently selected from nitrogen, oxygen, or sulfur, wherein each phenylene, arylene, carbocyclylene, heterocyclylene and heteroarylene is substituted with 0, 1, 2, 3, or 4 instances of R^{C};
LBM is selected from:

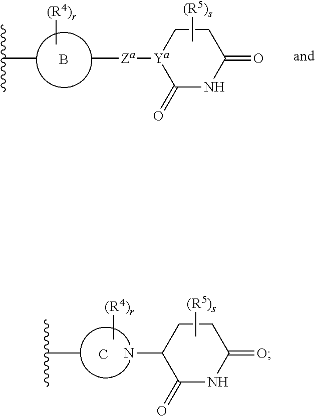

Y^{a} is CH or N;
Z^{a} is a bond, —CH₂—, —NH—, 0, or —NHC(O)— where NH of —NHC(O)— is attached to Y^{a};
Ring B is phenylene, a 4-10-membered heterocyclylene, a 5-6-membered monocyclic heteroarylene or a 9-10-membered fused bicyclic heteroarylene, wherein each heteroarylene contains one to three nitrogen ring atoms.
ring C together with the (R⁴)r substituents is selected from the group consisting of

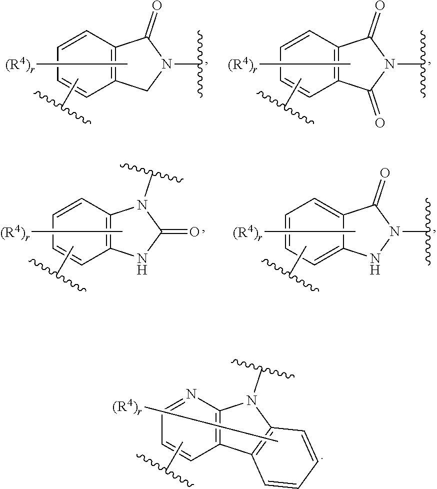

each instance of Re is independently selected from —D, halogen, —OH, and —C₁₋₆ alkyl;
each instance of R⁴ is independently selected from —D, halogen, —OH, and —C₁₋₆ alkyl;
each instance of R⁵ is independently selected from —D, halogen, —OH, and —C₁₋₆alkyl;
each instance of R is independently selected from hydrogen and —C₁₋₆alkyl;
n is 0, 1, 2, 3, or 4;
r is 0, 1, 2, 3, or 4; and
s is 0, 1, 2, 3, or 4.

Embodiment 2. The compound of embodiment 1, or a pharmaceutically acceptable salt thereof, wherein T is N and the compound is of formula A-I-A:

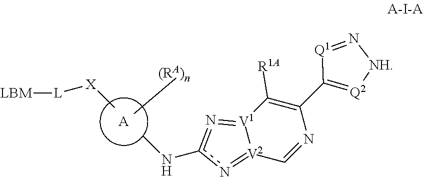

Embodiment 3. The compound of embodiment 1, or a pharmaceutically acceptable salt thereof, wherein T is CH and the compound is of formula A-I-B:

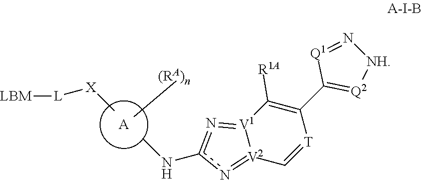

Embodiment 4. The compound of embodiment 1, or a pharmaceutically acceptable salt thereof, wherein the compound is of formula A-I-1:

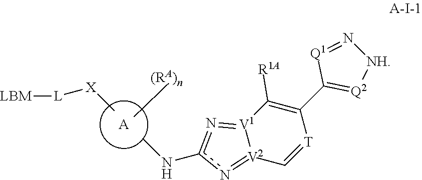

Embodiment 5. The compound of embodiment 1, or a pharmaceutically acceptable salt thereof, wherein the compound is of formula A-I-2:

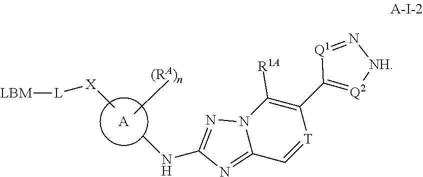

Embodiment 6. The compound of embodiment 1, or a pharmaceutically acceptable salt thereof, wherein the compound is of formula A-I-A1:

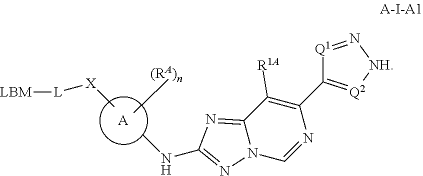

Embodiment 7. The compound of embodiment 1, or a pharmaceutically acceptable salt thereof, wherein the compound is of formula A-I-A2:

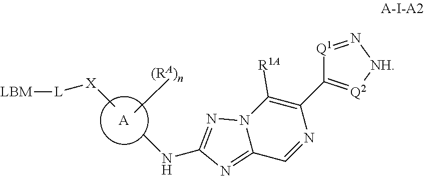

Embodiment 8. The compound of embodiment 1, or a pharmaceutically acceptable salt thereof, wherein the compound is of formula A-I-B1:

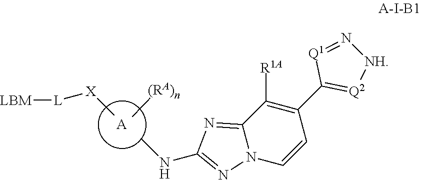

Embodiment 9. The compound of embodiment 1, or a pharmaceutically acceptable salt thereof, wherein the compound is of formula A-I-B2:

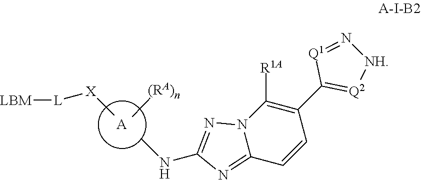

Embodiment 10. The compound of any one of embodiments 1-9, or a pharmaceutically acceptable salt thereof, wherein X is selected from —S(O)₂— and —C(O)—.

Embodiment 11. The compound of any one of embodiments 1-9, or a pharmaceutically acceptable salt thereof, wherein X is —S(O)₂—.

Embodiment 12. The compound of any one of embodiments 1-11, or a pharmaceutically acceptable salt thereof, wherein n is 0, 1 or 2.

Embodiment 13. The compound of any one of embodiments 1-11, or a pharmaceutically acceptable salt thereof, wherein n is 0 or 1.

Embodiment 14. The compound of any one of embodiments 1-11, or a pharmaceutically acceptable salt thereof, wherein n is 0.

Embodiment 15. The compound of any one of embodiments 1-11, or a pharmaceutically acceptable salt thereof, wherein n is 1.

Embodiment 16. The compound of any one of embodiments 1-11, or a pharmaceutically acceptable salt thereof, wherein n is 2.

Embodiment 17. The compound of any one of embodiments 1-16, or a pharmaceutically acceptable salt thereof, wherein Ring A is selected from

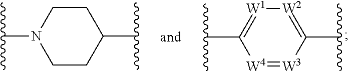

each W¹, W², W³ and W⁴ is independently selected from CH and N, provided that no more than 2 of W¹, W², W³ and W⁴ are N.

Embodiment 18. The compound of any one of embodiments 1-16, or a pharmaceutically acceptable salt thereof, wherein Ring A is selected from

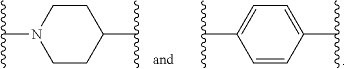 [SMILES: *C1CCN(*)CC1 ; *c1ccc(*)cc1]

Embodiment 19. The compound of any one of embodiments 1-16, or a pharmaceutically acceptable salt thereof, wherein Ring A is

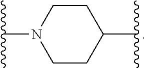 [SMILES: *C1CCN(*)CC1]

Embodiment 20. The compound of any one of embodiments 1-16, or a pharmaceutically acceptable salt thereof, wherein Ring A is

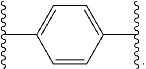 [SMILES: *c1ccc(*)cc1]

Embodiment 21. The compound of any one of embodiments 1-16, or a pharmaceutically acceptable salt thereof, wherein Ring A is

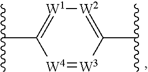

wherein each W¹, W², W³ and W⁴ is independently selected from CH and N, provided that no more than 2 of W¹, W², W³ and W⁴ are N.

Embodiment 22. The compound of any one of embodiments 1-16, or a pharmaceutically acceptable salt thereof, wherein Ring A is

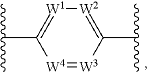

wherein one of W¹, W², W³ and W⁴ is independently N, and the others of W¹, W², W³ and W⁴ are CH.

Embodiment 23. The compound of any one of embodiments 1-16, or a pharmaceutically acceptable salt thereof, wherein Ring A is

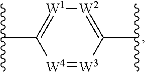

wherein two of W¹, W², W³ and W⁴ are independently N, and the other two of W¹, W², W³ and W⁴ are CH.

Embodiment 24. The compound of any one of embodiments 1-16, or a pharmaceutically acceptable salt thereof, wherein the compound is a compound of Formula A-IT:

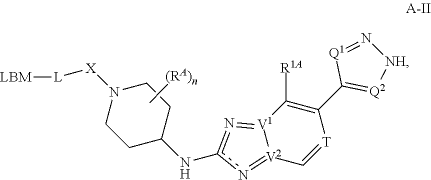

Embodiment 25. The compound of embodiment 24, or a pharmaceutically acceptable salt thereof, wherein the compound is of Formula A-IT-A:

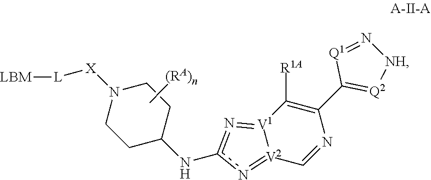

Embodiment 26. The compound of embodiment 24, or a pharmaceutically acceptable salt thereof, wherein the compound is of Formula A-II-B:

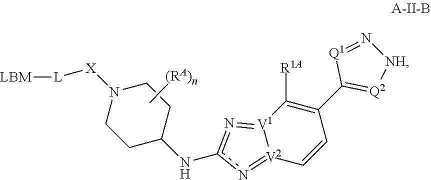

Embodiment 27. The compound of embodiment 24, or a pharmaceutically acceptable salt thereof, wherein the compound is of Formula A-II-1:

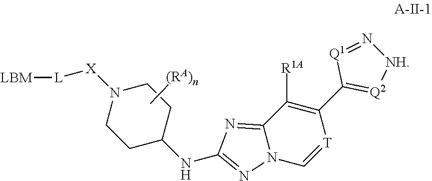

Embodiment 28. The compound of embodiment 24, or a pharmaceutically acceptable salt thereof, wherein the compound is of Formula A-II-2:

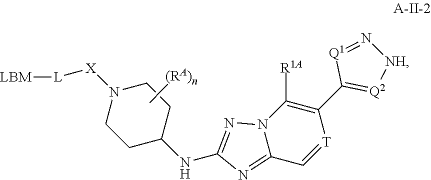

Embodiment 29. The compound of embodiment 24, or a pharmaceutically acceptable salt thereof, wherein the compound is of Formula A-IT-A1:

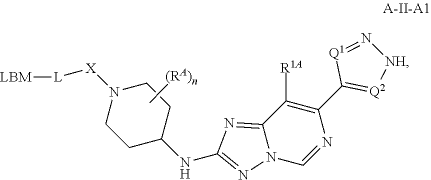

Embodiment 30. The compound of embodiment 24, or a pharmaceutically acceptable salt thereof, wherein the compound is of Formula A-II-A2:

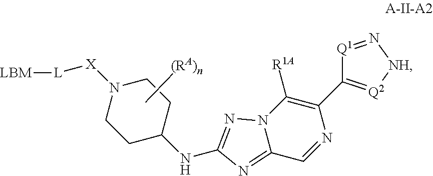

Embodiment 31. The compound of embodiment 24, or a pharmaceutically acceptable salt thereof, wherein the compound is of Formula A-II-Bi:

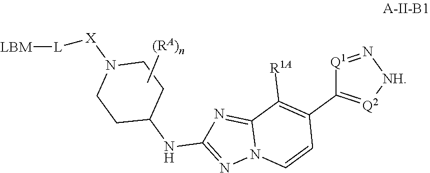

Embodiment 32. The compound of embodiment 24, or a pharmaceutically acceptable salt thereof, wherein the compound is of Formula A-II-B2:

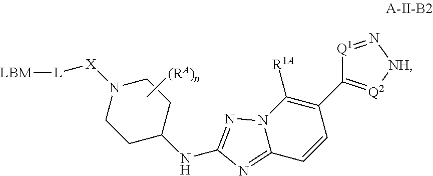

Embodiment 33. The compound of embodiment 24, or a pharmaceutically acceptable salt thereof, the compound is a compound of Formula A-III:

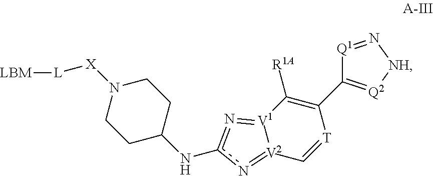

Embodiment 34. The compound of embodiment 24, or a pharmaceutically acceptable salt thereof, wherein the compound is of Formula A-III-A:

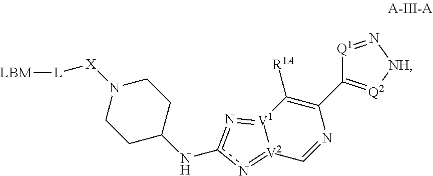

Embodiment 35. The compound of embodiment 24, or a pharmaceutically acceptable salt thereof, wherein the compound is of Formula A-ITT-B:

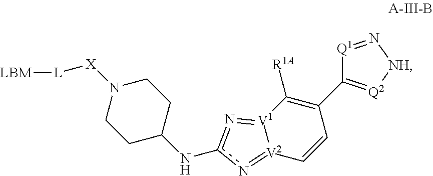

Embodiment 36. The compound of embodiment 24, or a pharmaceutically acceptable salt thereof, wherein the compound is of Formula A-III-1:

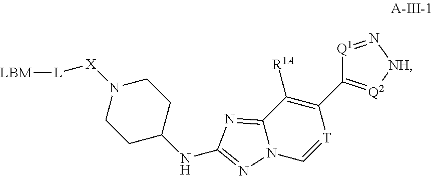

Embodiment 37. The compound of embodiment 24, or a pharmaceutically acceptable salt thereof, wherein the compound is of Formula A-III-2:

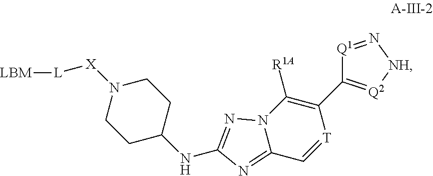

Embodiment 38. The compound of embodiment 24, or a pharmaceutically acceptable salt thereof, wherein the compound is of Formula A-ITT-A1:

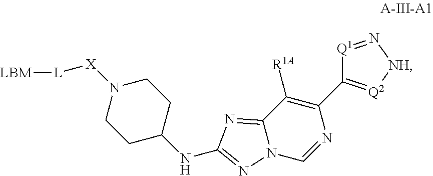

Embodiment 39. The compound of embodiment 24, or a pharmaceutically acceptable salt thereof, wherein the compound is of Formula A-III-A2:

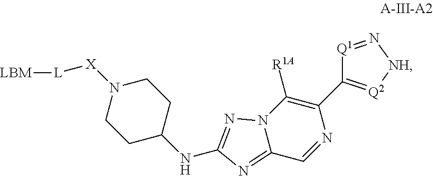

Embodiment 40. The compound of embodiment 24, or a pharmaceutically acceptable salt thereof, wherein the compound is of Formula A-ITT-Bi:

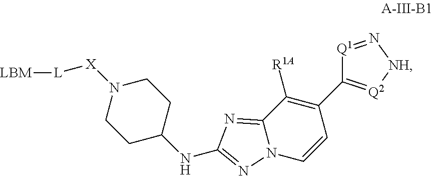

Embodiment 41. The compound of embodiment 24, or a pharmaceutically acceptable salt thereof, wherein the compound is of Formula A-III-B2:

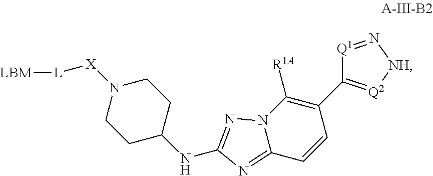

Embodiment 42. The compound of embodiment 24, or a pharmaceutically acceptable salt thereof, the compound is a compound of Formula A-IV:

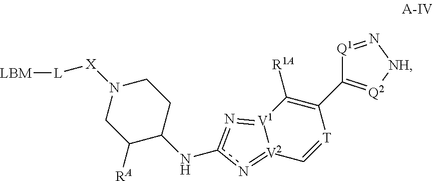

Embodiment 43. The compound of embodiment 24, or a pharmaceutically acceptable salt thereof, wherein the compound is of Formula A-IV-A:

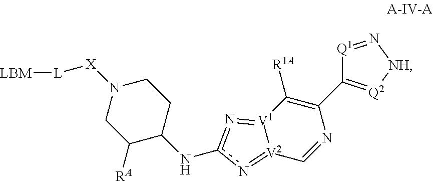

Embodiment 44. The compound of embodiment 24, or a pharmaceutically acceptable salt thereof, wherein the compound is of Formula A-IV-B:

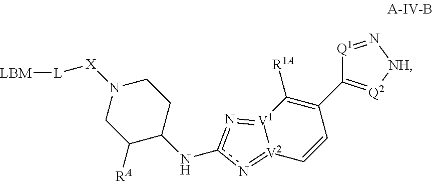

Embodiment 45. The compound of embodiment 24, or a pharmaceutically acceptable salt thereof, wherein the compound is of Formula A-IV-1:

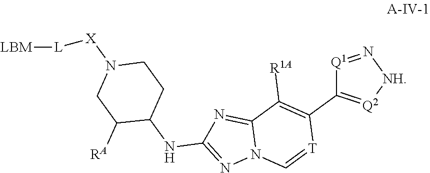

Embodiment 46. The compound of embodiment 24, or a pharmaceutically acceptable salt thereof, wherein the compound is of Formula A-IV-2:

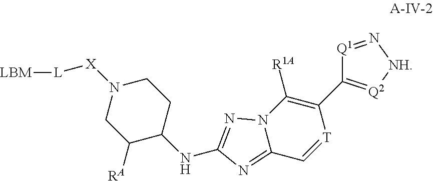

Embodiment 47. The compound of embodiment 24, or a pharmaceutically acceptable salt thereof, wherein the compound is of Formula A-IV-A1:

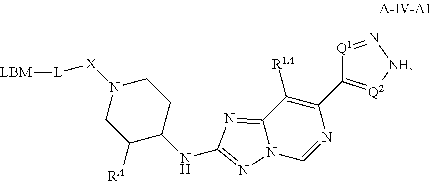

Embodiment 48. The compound of embodiment 24, or a pharmaceutically acceptable salt thereof, wherein the compound is of Formula A-IV-A2:

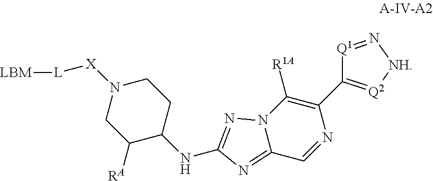

Embodiment 49. The compound of embodiment 24, or a pharmaceutically acceptable salt thereof, wherein the compound is of Formula A-IV-B1:

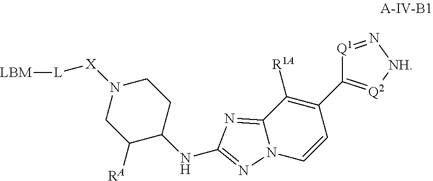

Embodiment 50. The compound of embodiment 24, or a pharmaceutically acceptable salt thereof, wherein the compound is of Formula IV-B2:

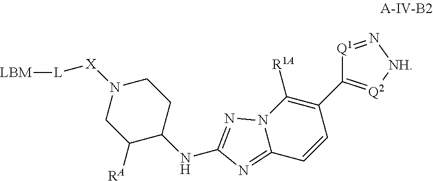

Embodiment 51. The compound of any one of embodiments 1-16, or a pharmaceutically acceptable salt thereof, wherein Ring A is

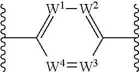

and the compound is a compound of Formula A-V:

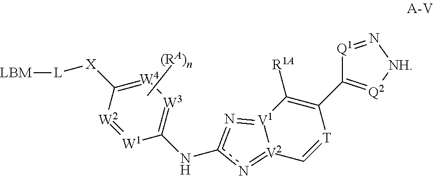

Embodiment 52. The compound of embodiment 51, or a pharmaceutically acceptable salt thereof, wherein the compound is of Formula A-V-A:

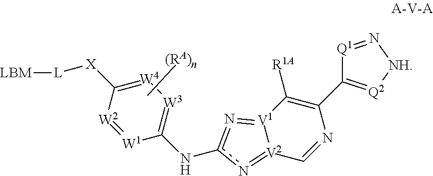

Embodiment 53. The compound of embodiment 51, or a pharmaceutically acceptable salt thereof, wherein the compound is of Formula A-V-B:

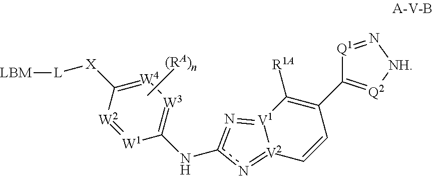

Embodiment 54. The compound of embodiment 51, or a pharmaceutically acceptable salt thereof, wherein the compound is a compound of Formula A-V-1:

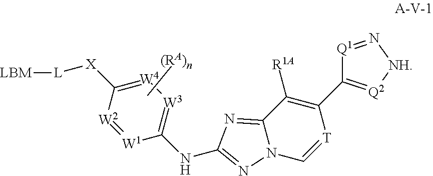

Embodiment 55. The compound of embodiment 51, or a pharmaceutically acceptable salt thereof, wherein the compound is a compound of Formula A-V-2:

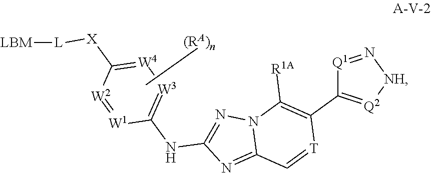

Embodiment 56. The compound of embodiment 51, or a pharmaceutically acceptable salt thereof, wherein the compound is a compound of Formula A-V-A1:

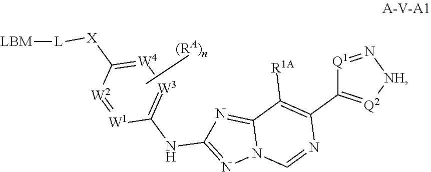

Embodiment 57. The compound of embodiment 51, or a pharmaceutically acceptable salt thereof, wherein the compound is a compound of Formula A-V-A2:

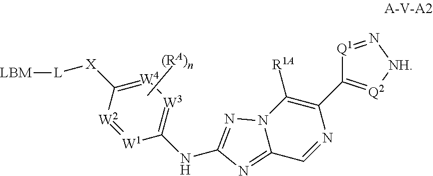

Embodiment 58. The compound of embodiment 51, or a pharmaceutically acceptable salt thereof, wherein the compound is a compound of Formula A-V-B1:

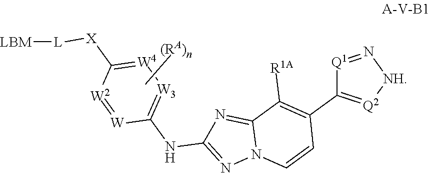

Embodiment 59. The compound of embodiment 51, or a pharmaceutically acceptable salt thereof, wherein the compound is a compound of Formula A-V-B2:

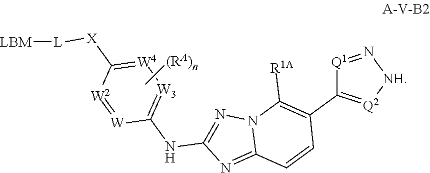

Embodiment 60. The compound of embodiment 51, or a pharmaceutically acceptable salt thereof, wherein the compound is a compound of Formula A-VI:

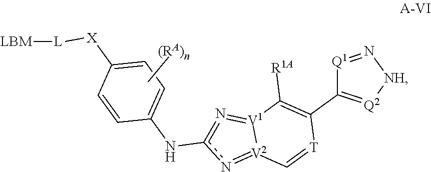

Embodiment 61. The compound of embodiment 51, or a pharmaceutically acceptable salt thereof, wherein the compound is of Formula A-VI-A:

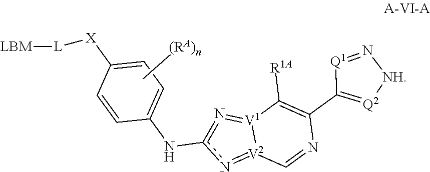

Embodiment 62. The compound of embodiment 51, or a pharmaceutically acceptable salt thereof, wherein the compound is of Formula A-VI-B:

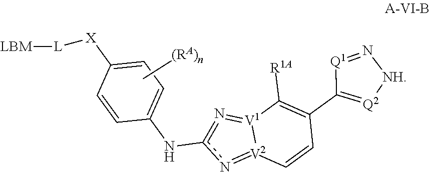

Embodiment 63. The compound of embodiment 51, or a pharmaceutically acceptable salt thereof, wherein the compound is a compound of Formula A-VI-1:

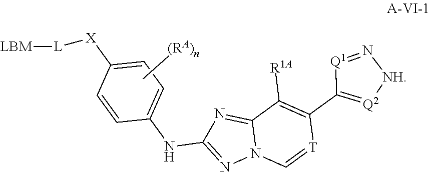

Embodiment 64. The compound of embodiment 51, or a pharmaceutically acceptable salt thereof, wherein the compound is a compound of Formula A-VI-2:

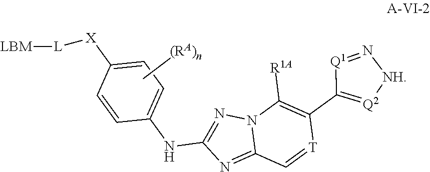

Embodiment 65. The compound of embodiment 51, or a pharmaceutically acceptable salt thereof, wherein the compound is a compound of Formula A-VI-A1:

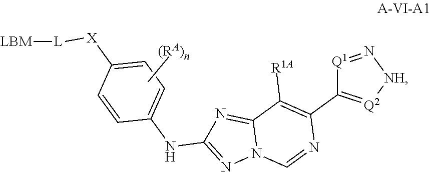

Embodiment 66. The compound of embodiment 51, or a pharmaceutically acceptable salt thereof, wherein the compound is a compound of Formula A-VI-A2:

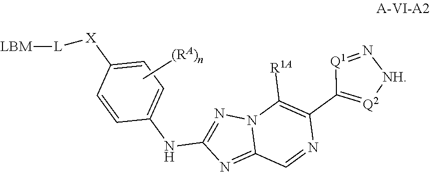

Embodiment 67. The compound of embodiment 51, or a pharmaceutically acceptable salt thereof, wherein the compound is a compound of Formula A-VI-B1:

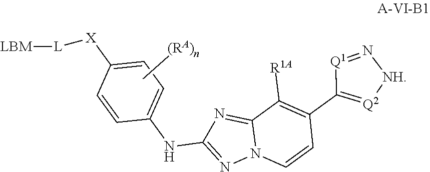

Embodiment 68. The compound of embodiment 51, or a pharmaceutically acceptable salt thereof, wherein the compound is a compound of Formula A-VI-B2:

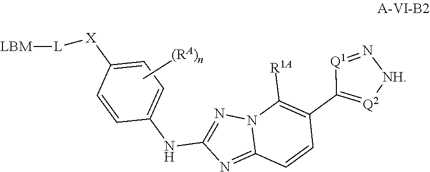

Embodiment 69. The compound of embodiment 51, or a pharmaceutically acceptable salt thereof, wherein the compound is a compound of Formula A-VII:

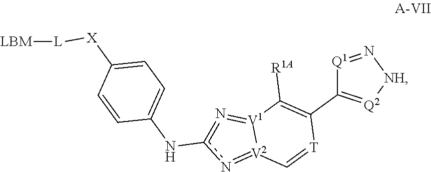

Embodiment 70. The compound of embodiment 51, or a pharmaceutically acceptable salt thereof, wherein the compound is of Formula A-VII-A:

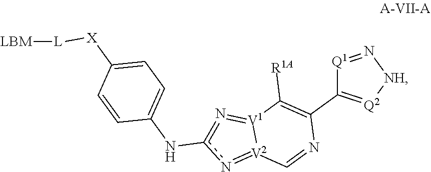

Embodiment 71. The compound of embodiment 51, or a pharmaceutically acceptable salt thereof, wherein the compound is of Formula A-VII-B:

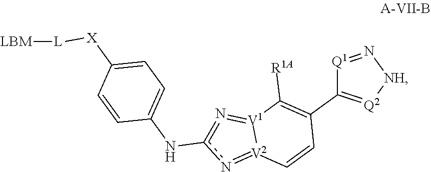

Embodiment 72. The compound of embodiment 51, or a pharmaceutically acceptable salt thereof, wherein the compound is a compound of Formula A-VII-1:

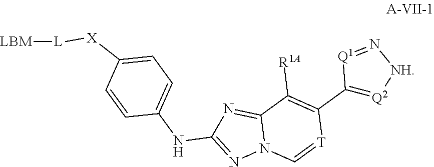

Embodiment 73. The compound of embodiment 51, or a pharmaceutically acceptable salt thereof, wherein the compound is a compound of Formula A-VII-2:

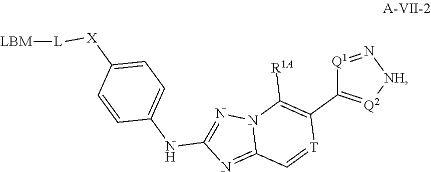

Embodiment 74. The compound of embodiment 51, or a pharmaceutically acceptable salt thereof, wherein the compound is a compound of Formula A-VII-A1:

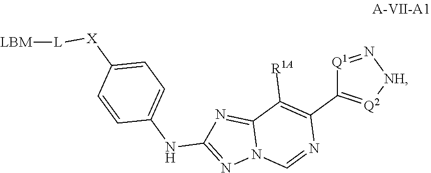

Embodiment 75. The compound of embodiment 51, or a pharmaceutically acceptable salt thereof, wherein the compound is a compound of Formula A-VII-A2:

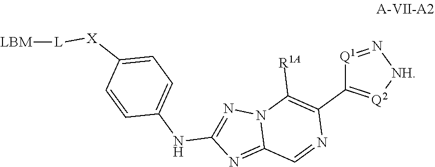

Embodiment 76. The compound of embodiment 51, or a pharmaceutically acceptable salt thereof, wherein the compound is a compound of Formula A-VII-B1:

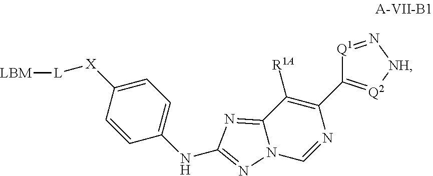

Embodiment 77. The compound of embodiment 51, or a pharmaceutically acceptable salt thereof, wherein the compound is a compound of Formula A-VII-B2:

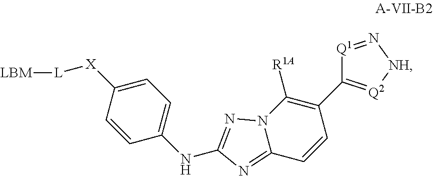

Embodiment 78. The compound of embodiment 51, or a pharmaceutically acceptable salt thereof, wherein the compound is a compound of Formula A-VIII:

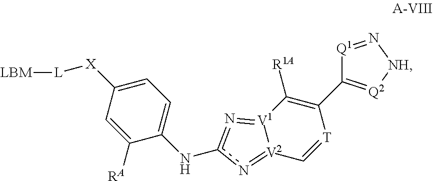

Embodiment 79. The compound of embodiment 51, or a pharmaceutically acceptable salt thereof, wherein the compound is of Formula A-VIII-A:

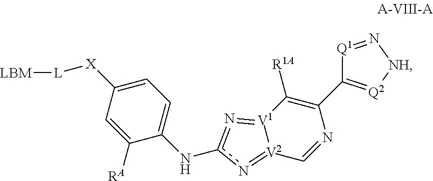

Embodiment 80. The compound of embodiment 51, or a pharmaceutically acceptable salt thereof, wherein the compound is of Formula A-VIII-B:

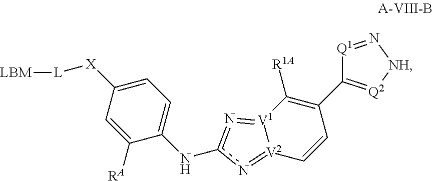

Embodiment 81. The compound of embodiment 51, or a pharmaceutically acceptable salt thereof, wherein the compound is a compound of Formula A-VIII-1:

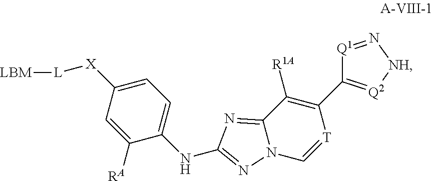

Embodiment 82. The compound of embodiment 51, or a pharmaceutically acceptable salt thereof, wherein the compound is a compound of Formula A-VIII-2:

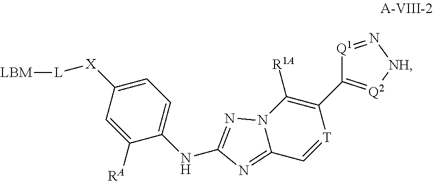

Embodiment 83. The compound of embodiment 51, or a pharmaceutically acceptable salt thereof, wherein the compound is a compound of Formula A-VIII-A1:

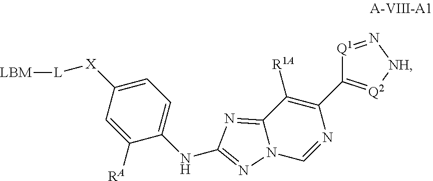

Embodiment 84. The compound of embodiment 51, or a pharmaceutically acceptable salt thereof, wherein the compound is a compound of Formula A-VIII-A2:

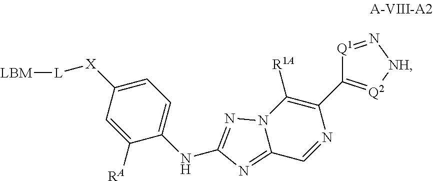

Embodiment 85. The compound of embodiment 51, or a pharmaceutically acceptable salt thereof, wherein the compound is a compound of Formula A-VIII-B1:

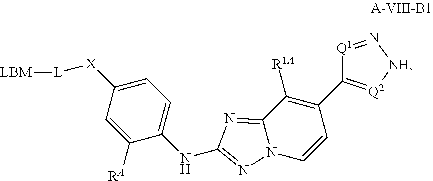

Embodiment 86. The compound of embodiment 51, or a pharmaceutically acceptable salt thereof, wherein the compound is a compound of Formula A-VIII-B2:

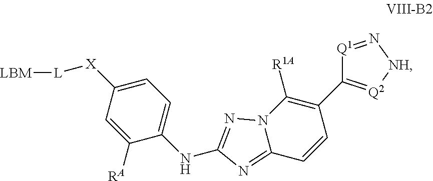

Embodiment 87. The compound of any one of embodiments 1-86, or a pharmaceutically acceptable salt thereof, wherein Q¹ and Q² are both CH.

Embodiment 88. The compound of any one of embodiments 1-86, or a pharmaceutically acceptable salt thereof, wherein Q¹ and Q² are both N.

Embodiment 89. The compound of any one of embodiments 1-86, or a pharmaceutically acceptable salt thereof, wherein Q¹ is N and Q² is CH.

Embodiment 90. The compound of any one of embodiments 1-91, or a pharmaceutically acceptable salt thereof, wherein Q² is N and Q¹ is CH.

Embodiment 91. The compound of any one of embodiments 1-90, or a pharmaceutically acceptable salt thereof, wherein R^{1A} is independently selected from H, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocyclyl, 5-6 membered heteroaryl, C₃₋₇ cycloalkyl-C₁₋₄ alkyl-, phenyl-C₁₋₄ alkyl-, 4-7 membered heterocyclyl-C₁₋₄ alkyl-, 5-6 membered heteroaryl-C₁₋₄ alkyl-, OR^{a1}, SR^{a1} C(O)R^{b1}, C(O)NR^{a1}R^{a1}, C(O)OR^{a1}, OC(O)R^{b1}, OC(O)NR^{a1}R^{a1}, NR^{a1}R^{a1}, NR^{a1}C(O)R^{b1}, NR^{a1}C(O)OR^{a1}, NR^{a1}C(O)NR^{a1}R^{a1}, NR^{a1}S(O)₂R^{b1}, NR^{a1}S(O)₂NR^{a1}R^{a1}, S(O)₂R^{b1}, and 5 S(O)₂NR^{a1}R^{a1}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocyclyl, 5-6 membered heteroaryl, C₃₋₇ cycloalkyl-C₁₋₄ alkyl-, phenyl-C₁₋₄ alkyl-, 4-7 membered heterocyclyl-C₁₋₄ alkyl-, and 5-6 membered heteroaryl-C₁₋₄ alkyl- are each substituted with 0, 1, 2, 3, or 4 independently selected R² substituents.

Embodiment 92. The compound of any one of embodiments 1-90, or a pharmaceutically acceptable salt thereof, wherein R^{1A} is independently selected from H, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocyclyl, 5-6 membered heteroaryl, C₃₋₇ cycloalkyl-C₁₋₄ alkyl-, phenyl-C₁₋₄ alkyl-, 4-7 membered heterocyclyl-C₁₋₄ alkyl-, 5-6 membered heteroaryl-C₁₋₄ alkyl-, OR^{a1}, C(O)R^{b1}, C(O)NR^{a1}R^{a1}, C(O)OR^{a1}, OC(O)R^{b1}, OC(O)NR^{a1}R^{a1}, NR^{a1}R^{a1}, NR^{a1}C(O)R^{b1}, NR^{a1}C(O)OR^{a1}, NR^{a1}C(O)NR^{a1}R^{a1}, NR^{a1}S(O)₂R^{b1}, NR^{a1}S(O)₂NR^{a1}R^{a1}, S(O)₂R^{b1}, and S(O)₂NR^{a1}R^{a1}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocyclyl, 5-6 membered heteroaryl, C₃₋₇ cycloalkyl-C₁₋₄ alkyl-, phenyl-C₁₋₄ alkyl-, 4-7 membered heterocyclyl-C₁₋₄ alkyl-, and 5-6 membered heteroaryl-C₁₋₄ alkyl- are each substituted with 0, 1, 2, 3, or 4 independently selected R² substituents.

Embodiment 93. The compound of any one of embodiments 1-90, or a pharmaceutically acceptable salt thereof, wherein R^{1A} is independently selected from H, halo, CN, C₁₋₆ alkyl, C₁₋₆ haloalkyl, C₃₋₇ cycloalkyl, phenyl, 4-7 membered heterocyclyl, 5-6 membered heteroaryl, C₃₋₇ cycloalkyl-C₁₋₃ alkyl, phenyl-C₁₋₃ alkyl, 4-7 membered heterocyclyl-C₁₋₃ alkyl, 5-6 membered heteroaryl-C₁₋₃ alkyl, OR^{a1}, SR^{a1}, and NR^{a}R^{a1}, wherein said C₁₋₆ alkyl, C₁₋₆ haloalkyl, C₃₋₇ cycloalkyl, phenyl, 4-7 membered heterocyclyl, 5-6 membered heteroaryl, C₃₋₇ cycloalkyl-C₁₋₃ alkyl-, phenyl-C₁₋₃ alkyl-, 4-7 membered heterocyclyl-C₁₋₃ alkyl-, and 5-6 membered heteroaryl-C₁₋₃ alkyl are each substituted with 0, 1 or 2 independently selected R² substituents.

Embodiment 94. The compound of any one of embodiments 1-90, or a pharmaceutically acceptable salt thereof, wherein R^{1A} is independently selected from H, halo, CN, C₁₋₆ alkyl, C₁₋₆ haloalkyl, C₃₋₇ cycloalkyl, phenyl, 4-7 membered heterocyclyl, 5-6 membered heteroaryl, C₃₋₇ cycloalkyl-C₁₋₃ alkyl-, phenyl-C₁₋₃ alkyl-, 4-7 membered heterocyclyl-C₁₋₃ alkyl-, 5-6 membered heteroaryl-C₁₋₃ alkyl-, OR^{a1} and NR^{a1}R^{a1}, wherein said C₁₋₆ alkyl, C₁₋₆ haloalkyl, C₃₋₇ cycloalkyl, phenyl, 4-7 membered heterocyclyl, 5-6 membered heteroaryl, C₃₋₇ cycloalkyl-C₁₋₃ alkyl-, phenyl-C₁₋₃ alkyl-, 4-7 membered heterocyclyl-C₁₋₃ alkyl-, and 5-6 membered heteroaryl-C₁₋₃ alkyl are each substituted with 0, 1 or 2 independently selected R² substituents.

Embodiment 95. The compound of any one of embodiments 1-90, or a pharmaceutically acceptable salt thereof, wherein R^{1A} is independently selected from H, halo, CN, C₁₋₆ alkyl, C₂₋₆ alkenyl, C₂₋₆ alkynyl, C₁₋₆ haloalkyl, C₃-4 cycloalkyl, and OR^{a1}, wherein said C₁₋₆ alkyl, C₂₋₆ alkenyl, C₂₋₆ alkynyl, C₁₋₆ haloalkyl, and C₃₋₄ cycloalkyl are each substituted with 0, 1, 2, 3, or 4 independently selected R² substituents.

Embodiment 96. The compound of any one of embodiments 1-90, or a pharmaceutically acceptable salt thereof, wherein R^{1A} is independently selected from H, halo, CN, C₁₋₆ alkyl, C₂₋₆ alkenyl, C₂₋₆ alkynyl, C₁₋₆ haloalkyl, C₃-4 cycloalkyl, and OR^{a1}, wherein said C₁₋₆ alkyl, C₂₋₆ alkenyl, C₂₋₆ alkynyl, C₁₋₆ haloalkyl, and C₃₋₄ cycloalkyl are each substituted with 0, 1 or 2 independently selected R² substituents.

Embodiment 97. The compound of any one of embodiments 1-90, or a pharmaceutically acceptable salt thereof, wherein R^{1A} is independently selected from H, C₁₋₆ alkyl, phenyl, 5-7 membered heterocyclyl, OR^{a1}, SR^{a1}, and NR^{a1}R^{a1}, wherein said C₁₋₆ alkyl, phenyl, and 5-7 membered heterocyclyl are each substituted with 0, 1 or 2 independently selected R² substituents.

Embodiment 98. The compound of any one of embodiments 1-90, or a pharmaceutically acceptable salt thereof, wherein R^{1A} is independently selected from H, halo, CN, C₁₋₆ alkyl, C₂₋₆ alkenyl, C₂₋₆ alkynyl, C₁₋₆ haloalkyl, C₃₋₇ cycloalkyl, phenyl, 4-7 membered heterocyclyl, 5-6 membered heteroaryl, C₃₋₇ cycloalkyl-C₁₋₄ alkyl-, phenyl-C₁₋₄ alkyl-, 4-7 membered heterocyclyl-C₁₋₄ alkyl-, 5-6 membered heteroaryl-C₁₋₄ alkyl-, OR^{a}, SR^{a1}, and NR^{a1}R^{a1} wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocyclyl, 5-6 membered heteroaryl, C₃₋₇ cycloalkyl-C₁₋₄ alkyl-, phenyl-C₁₋₄ alkyl-, 4-7 membered heterocyclyl-C₁₋₄ alkyl-, and 5-6 membered heteroaryl-C₁₋₄ alkyl- are each substituted with 0, 1, 2, 3, or 4 independently selected R² substituents.

Embodiment 99. The compound of any one of embodiments 1-90, or a pharmaceutically acceptable salt thereof, wherein R^{1A} is H, C₁₋₆ alkyl, phenyl, 5-7 membered heterocyclyl, OR^{a1}, SR^{a1}, or NR^{a}R^{a1}, wherein said C₁₋₆ alkyl, phenyl, and 5-7 membered heterocyclyl are each substituted with 0, 1 or 2 independently selected R² substituents

Embodiment 100. The compound of any one of embodiments 1-90, or a pharmaceutically acceptable salt thereof, wherein R^{1A} is phenyl substituted with 0, 1 or 2 independently selected R² substituents.

Embodiment 101. The compound of any one of embodiments 1-90, wherein R^{1A} is selected from 6-10 membered aryl, 4-10 membered heterocyclyl, 5-10 membered heteroaryl, OR^{a1} and NR^{a1}R^{a1}, wherein said 6-10 membered aryl, 4-10 membered heterocyclyl, and 5-10 membered heteroaryl are each substituted with 0, 1, 2, 3 or 4 independently selected R² substituents.

Embodiment 102. The compound of any one of embodiments 1-90, or a pharmaceutically acceptable salt thereof, wherein R^{1A} is independently selected from phenyl, 4-10 membered heterocyclyl, 5-10 membered heteroaryl, OR^{a1}, and NR^{a1}R^{a1}, wherein said phenyl, 4-10 membered heterocyclyl, and 5-10 membered heteroaryl are each substituted with 0, 1, 2, 3, or 4 independently selected R² substituents.

Embodiment 103. The compound of any one of embodiments 1-90, or a pharmaceutically acceptable salt thereof, wherein R^{1A} is independently selected from 4-10 membered heterocyclyl and OR^{a1}, wherein said 4-10 membered heterocyclyl is substituted with 0, 1, 2, 3, or 4 independently selected R² substituents.

Embodiment 104. The compound of any one of embodiments 1-90, or a pharmaceutically acceptable salt thereof, wherein R^{1A} is 5-7 membered heterocyclyl substituted with 0, 1 or 2 independently selected R² substituents.

Embodiment 105. The compound of any one of embodiments 1-90, or a pharmaceutically acceptable salt thereof, wherein R^{1A} is independently selected from phenyl, 4-10 membered heterocyclyl, 5-10 membered heteroaryl, and OR^{a1}, wherein said phenyl, 4-10 membered heterocyclyl, and 5-10 membered heteroaryl are each substituted with 0, 1, 2, 3, or 4 independently selected R² substituents.

Embodiment 106. The compound of any one of embodiments 1-90, or a pharmaceutically acceptable salt thereof, wherein R^{1A} is selected from OR^{a1}, phenyl, pyridin-4-yl, 2-oxy-benzo[d]oxazol-(3H)-7-yl, and 1H-indazol-5-yl, wherein said phenyl, pyridin-4-yl, 2-oxy-benzo[d]oxazol-(3H)-7-yl, and 1H-indazol-5-yl are each substituted with 0, 1, 2, 3, or 4 independently selected R² substituents.

Embodiment 107. The compound of any one of embodiments 1-90, or a pharmaceutically acceptable salt thereof, wherein R^{1A} is selected from phenyl, 5-10 membered heteroaryl, and OR^{a1}, wherein said phenyl and 5-10 membered heteroaryl are each substituted with 0, 1, 2, 3, or 4 independently selected R² substituents.

Embodiment 108. The compound of any one of embodiments 1-107, or a pharmaceutically acceptable salt thereof, wherein each R^{b1} is independently selected from C₁₋₆ alkyl and C₃₋₉ cycloalkyl.

Embodiment 109. The compound of any one of embodiments 1-107, or a pharmaceutically acceptable salt thereof, wherein each R^{b1} is independently selected from C₁₋₆ alkyl and C₃₋₆ cycloalkyl.

Embodiment 110. The compound of any one of embodiments 1-107, or a pharmaceutically acceptable salt thereof, wherein each R^{b1} is independently selected from —Me, —Et, —iPr, cyclopropyl, cyclobutyl, cyclopentyl, cyclohexyl.

Embodiment 111. The compound of any one of embodiments 1-107, or a pharmaceutically acceptable salt thereof, wherein each R^{b1} is independently selected from —Me, —Et and ′Pr.

Embodiment 112. The compound of any one of embodiments 1-107, or a pharmaceutically acceptable salt thereof, wherein each R^{b1} is independently —Me.

Embodiment 113. The compound of any one of embodiments 1-112, or a pharmaceutically acceptable salt thereof, wherein R^{a1} is selected from H, C₁₋₆ alkyl, C₁₋₆ haloalkyl, C₃₋₇ cycloalkyl, phenyl, 4-7 membered heterocyclyl, 5-6 membered heteroaryl, C₃₋₇ cycloalkyl-C₁₋₄ alkyl-, phenyl-C₁₋₄ alkyl-, 4-7 membered heterocyclyl-C₁₋₄ alkyl-, and 5-6 membered heteroaryl-C₁₋₄ alkyl-, wherein said C₁₋₆ alkyl, C₁₋₆ haloalkyl, C₃₋₇ cycloalkyl, phenyl, 4-7 membered heterocyclyl, 5-6 membered heteroaryl, C₃₋₇ cycloalkyl-C₁₋₄ alkyl-, phenyl-C₁₋₄ alkyl-, 4-7 membered heterocyclyl-C₁₋₄ alkyl-, and 5-6 membered heteroaryl-C₁₋₄ alkyl are each substituted with 0, 1, 2, 3, or 4 independently selected R² substituents.

Embodiment 114. The compound of any one of embodiments 1-112, or a pharmaceutically acceptable salt thereof, wherein R^{a1} is selected from H, C₁₋₆ alkyl, C₁₋₆ haloalkyl, C₃₋₆ cycloalkyl, phenyl, 4-6 membered heterocyclyl, C₃₋₆ cycloalkyl-C₁₋₃ alkyl-, and 4-6 membered heterocyclyl-C₁₋₃ alkyl-, wherein said C₁₋₆ alkyl, C₁₋₆ haloalkyl, C₃₋₆ cycloalkyl, phenyl, 4-6 membered heterocyclyl, C₃₋₆ cycloalkyl-C₁₋₃ alkyl-, and 4-6 membered heterocyclyl-C₁₋₃ alkyl- are each substituted with 0, 1, 2, or 3 independently selected R² substituents.

Embodiment 115. The compound of any one of embodiments 1-112, or a pharmaceutically acceptable salt thereof, wherein R^{a1} is selected from H, C₁₋₆ alkyl, C₁₋₆ haloalkyl, C₃₋₇ cycloalkyl, phenyl, 4-7 membered heterocyclyl, and C₃₋₇ cycloalkyl-C₁₋₄ alkyl, wherein said C₁₋₆ alkyl, C₁₋₆ haloalkyl, C₃₋₇ cycloalkyl, phenyl, 4-7 membered heterocyclyl, and C₃₋₇ cycloalkyl-C₁₋₄ alkyl are each substituted with 0, 1, 2 or 3 independently selected R² substituents.

Embodiment 116. The compound of any one of embodiments 1-112, or a pharmaceutically acceptable salt thereof, wherein R^{a1} is selected from H, C₁₋₆ alkyl, C₁₋₆ haloalkyl, C₃₋₆ cycloalkyl, 4-6 membered heterocyclyl, C₃₋₆ cycloalkyl-C₁₋₃ alkyl-, and 4-6 membered heterocyclyl-C₁₋₃ alkyl-, wherein said C₁₋₆ alkyl, C₁₋₆ haloalkyl, C₃₋₆ cycloalkyl, 4-6 membered heterocyclyl, C₃₋₆ cycloalkyl-C₁₋₃ alkyl-, and 4-6 membered heterocyclyl-C₁₋₃ alkyl- are each substituted with 0, 1 or 2 independently selected R² substituents.

Embodiment 117. The compound of any one of embodiments 1-112, or a pharmaceutically acceptable salt thereof, wherein R^{a1} is selected from C₁₋₆ alkyl, C₁₋₆ haloalkyl and C₃₋₆ cycloalkyl, wherein said C₁₋₆ alkyl, C₁₋₆ haloalkyl and C₃₋₆ cycloalkyl are each substituted with 0, 1 or 2 independently selected R² substituents.

Embodiment 118. The compound of any one of embodiments 1-112, or a pharmaceutically acceptable salt thereof, wherein R^{a1} is selected from C₁₋₆ alkyl, C₁₋₆ haloalkyl and C₃₋₆ cycloalkyl, wherein said C₁₋₆ alkyl, C₁₋₆ haloalkyl and C₃₋₆ cycloalkyl are each substituted with 0, 1 or 2 independently selected R² substituents.

Embodiment 119. The compound of any one of embodiments 1-112, or a pharmaceutically acceptable salt thereof, wherein R^{a1} is selected from C₁₋₄ alkyl, C₁₋₃ fluoroalkyl and C₃₋₆ cycloalkyl, wherein said C₁₋₄ alkyl, C₁₋₃ fluoroalkyl and C₃₋₆ cycloalkyl are each substituted with 0, 1 or 2 independently selected R² substituents.

Embodiment 120. The compound of any one of embodiments 1-112, or a pharmaceutically acceptable salt thereof, wherein R^{a1} is selected from —Me, —Et, —Pr, —′Pr, iso-butyl, sec-butyl, -butyl, cyclopropyl, cyclobutyl, cyclopentyl, —CF₃, —CHF₂, CH₂CF₃, cyclopropyl, cyclobutyl, cyclopentyl and cyclohexyl, each substituted with 0, 1 or 2 independently selected R² substituents. Embodiment 121. The compound of any one of embodiments 1-120, or a pharmaceutically acceptable salt thereof, wherein each R² is independently selected from H, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocyclyl, 5-6 membered heteroaryl, C₃₋₇ cycloalkyl-C₁₋₄ alkyl, phenyl-C₁₋₄ alkyl, 4-7 membered heterocyclyl-C₁₋₄ alkyl, 5-6 membered heteroaryl-C₁₋₄ alkyl, OR^{a2}, C(O)R^{b2}, C(O)NR^{a2}R^{a2}, C(O)OR^{a2}, OC(O)R^{b2}, OC(O)NR^{a2}R^{a2}, NR^{a2}R^{a2}, NR^{a2}C(O)R^{b2}, NR^{a2}C(O)OR^{a2}, NR^{a2}C(O)NR^{a2}R^{a2}, NR^{a2}S(O)₂R^{b2}, NR^{a2}S(O)₂NR^{a2}R^{a2}, S(O)₂R^{b2}, and S(O)₂NR^{a2}R^{a2}, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocyclyl, 5-6 membered heteroaryl, C₃₋₇ cycloalkyl-C₁₋₄ alkyl, phenyl-C₁₋₄ alkyl, 4-7 membered heterocyclyl-C₁₋₄ alkyl, and 5-6 membered heteroaryl-C₁₋₄ alkyl are each substituted with 0, 1, 2, 3, or 4 substituents independently selected from C₁₋₄ alkyl, C₃₋₇ cycloalkyl, cyclopropyl, oxo, —C(O)C₁₋₄alkyl, —C(O)OC₁₋₄alkyl, —C(O)NH₂, —OH, —F, —Cl, —O—C₁₋₄alkyl and —CN.

Embodiment 122. The compound of any one of embodiments 1-120, or a pharmaceutically acceptable salt thereof, wherein each R² is independently selected from H, D, halo, CN, NO₂, C₁₋₆ alkyl, C₁₋₆ haloalkyl, C₃₋₇ cycloalkyl, phenyl, 4-7 membered heterocyclyl, 5-6 membered heteroaryl, C₃₋₇ cycloalkyl-C₁₋₄ alkyl, phenyl-C₁₋₄ alkyl, 4-7 membered heterocyclyl-C₁₋₄ alkyl, 5-6 membered heteroaryl-C₁₋₄ alkyl, OR^{a2}, C(O)R^{b2}, C(O)NR^{a2}R^{a2}, C(O)OR^{a2}, OC(O)R^{b2}, OC(O)NR^{a2}R^{a2}, NR^{a2}R^{a2}, NR^{a2}C(O)R^{b2}, NR^{a2}C(O)OR^{a2}, NR^{a2}C(O)NR^{a2}R^{a2}, NR^{a2}S(O)₂R^{b2}, NR^{a2}S(O)₂NR^{a2}R^{a2}, S(O)₂R^{b2}, and S(O)₂NR^{a2}R^{a2}, wherein said C₁₋₆ alkyl, C₁₋₆ haloalkyl, C₃₋₇ cycloalkyl, phenyl, 4-7 membered heterocyclyl, 5-6 membered heteroaryl, C₃₋₇ cycloalkyl-C₁₋₄ alkyl, phenyl-C₁₋₄ alkyl, 4-7 membered heterocyclyl-C₁₋₄ alkyl, and 5-6 membered heteroaryl-C₁₋₄ alkyl are each substituted with 0, 1, 2, 3, or 4 substituents independently selected from C₁₋₄ alkyl, C₃₋₇ cycloalkyl, cyclopropyl, oxo, —C(O)C₁₋₄alkyl, —C(O)OC₁₋₄alkyl, —C(O)NH₂, —OH, —F, —Cl, —O—C₁₋₄alkyl and —CN.

Embodiment 123. The compound of any one of embodiments 1-120, or a pharmaceutically acceptable salt thereof, wherein each R² is independently selected from H, halo, CN, NO₂, C₁₋₆ alkyl, C₁₋₆ haloalkyl, C₃₋₇ cycloalkyl, phenyl, 4-7 membered heterocyclyl, 5-6 membered heteroaryl, C₃₋₇ cycloalkyl-C₁₋₄ alkyl, phenyl-C₁₋₄ alkyl, 4-7 membered heterocyclyl-C₁₋₄ alkyl, 5-6 membered heteroaryl-C₁₋₄ alkyl, OR^{a2}, C(O)R^{b2}, C(O)NR^{a2}R^{a2}, C(O)OR^{a2}, OC(O)R^{b2}, OC(O)NR^{a2}R^{a2}, NR^{a2}R^{a2}, NR^{a2}C(O)R^{b2}, NR^{a2}C(O)OR^{a2}, NR^{a2}C(O)NR^{a2}R^{a2}, NR^{a2}S(O)₂R^{b2}, NR^{a2}S(O)₂NR^{a2}R^{a2}, S(O)₂R^{b2}, and S(O)₂NR^{a2}R^{a2}.

Embodiment 124. The compound of any one of embodiments 1-120, or a pharmaceutically acceptable salt thereof, wherein each R² is independently selected from halo, CN, C₁₋₆ alkyl, C₁₋₆ haloalkyl, C₃₋₆ cycloalkyl, 4-6 membered heterocyclyl, C₃₋₆ cycloalkyl-C₁₋₄ alkyl, 4-6 membered heterocyclyl-C₁₋₄ alkyl, OR^{a2}, NR^{a2}R^{a2} and S(O)₂R^{b2}, wherein said C₁₋₆ alkyl, C₁₋₆ haloalkyl, C₃₋₆ cycloalkyl, 4-6 membered heterocyclyl, C₃₋₆ cycloalkyl-C₁₋₄ alkyl, and 4-6 membered heterocyclyl-C₁₋₄ alkyl are each substituted with 0, 1, 2, 3, or 4 substituents independently selected from C₁₋₄ alkyl, C₃₋₇ cycloalkyl, cyclopropyl, oxo, —C(O)C₁₋₄alkyl, —C(O)OC₁₋₄alkyl, —C(O)NH₂, —OH, —F, —Cl, —O—C₁₋₄alkyl and —CN.

Embodiment 125. The compound of any one of embodiments 1-120, or a pharmaceutically acceptable salt thereof, wherein each R² is independently selected from halo, CN, C₁₋₆ alkyl, and 4-6 membered heterocyclyl-C₁₋₄ alkyl, wherein said C₁₋₆ alkyl and 4-6 membered heterocyclyl-C₁₋₄ alkyl are each substituted with 0, 1, 2, 3, or 4 substituents independently selected from C₁₋₄ alkyl, C₃₋₇ cycloalkyl, cyclopropyl, oxo, —C(O)C₁₋₄alkyl, —C(O)OC₁₋₄alkyl, —C(O)NH₂, —OH, —F, —Cl, —O—C₁₋₄alkyl and —CN.

Embodiment 126. The compound of any one of embodiments 1-120, or a pharmaceutically acceptable salt thereof, wherein each R² is independently selected from H, halo, CN, C₁₋₄ alkyl, C₁₋₄ haloalkyl, OH, C₁₋₃ alkoxy, C₁₋₃ haloalkoxy, amino, C₁₋₃ alkylamino, di(C₁₋₃ alkyl)amino, cyano-C₁₋₄ alkyl, HO—C₁₋₄ alkyl, C₁₋₃ alkoxy-C₁₋₄ alkyl, and C₃₋₄ cycloalkyl.

Embodiment 127. The compound of any one of embodiments 1-120, or a pharmaceutically acceptable salt thereof, wherein each R² is independently selected from H, D, halo, CN, C₁₋₆ alkyl, C₁₋₆ haloalkyl, OR^{a2}, and C(O)OR^{a2}, wherein said C₁₋₆ alkyl and C₁₋₆ haloalkyl are each substituted with 0, 1, 2, 3, or 4 substituents independently selected from C₁₋₄ alkyl, C₃₋₇ cycloalkyl, cyclopropyl, oxo, —C(O)C₁₋₄alkyl, —C(O)OC₁₋₄alkyl, —C(O)NH₂, —OH, —F, —Cl, —O—C₁₋₄alkyl and —CN.

Embodiment 128. The compound of any one of embodiments 1-120, or a pharmaceutically acceptable salt thereof, wherein each R² is independently selected from H, D, halo, CN, C₁₋₆ alkyl, C₁₋₆ haloalkyl, C₃₋₄ cycloalkyl, OR^{a2}, C(O)R^{b2}, C(O)NR^{a2}R^{a2}, C(O)OR^{a2}, OC(O)R^{b2}, OC(O)NR^{a2}R^{a2}, NR^{a2}R^{a2}, NR^{a2}C(O)R^{b2}, NR^{a2}C(O)OR^{a2}, NR^{a2}C(O)NR^{a2}R^{a2}, NR^{a2}S(O)₂R^{b2}, NR^{a2}S(O)₂NR^{a2}R^{a2}, S(O)₂R^{b2}, and S(O)₂NR^{a2}R^{a2}, wherein said C₁₋₆ alkyl, C₁₋₆ haloalkyl, and C₃₋₄ cycloalkyl are each substituted with 0, 1, 2, 3, or 4 substituents independently selected from C₁₋₄ alkyl, C₃₋₇ cycloalkyl, cyclopropyl, oxo, —C(O)C₁₋₄alkyl, —C(O)OC₁₋₄alkyl, —C(O)NH₂, —OH, —F, —Cl, —O—C₁₋₄alkyl and —CN.

Embodiment 129. The compound of any one of embodiments 1-120, or a pharmaceutically acceptable salt thereof, wherein each R² is independently selected from D, halo, CN, C₁₋₄ alkyl, C₁₋₄ haloalkyl, OR^{a2}, and C(O)OR^{a2}, wherein said C₁₋₄ alkyl and C₁₋₄ haloalkyl are each substituted with 0, 1, 2, 3, or 4 substituents independently selected from C₁₋₄ alkyl, C₃₋₇ cycloalkyl, cyclopropyl, oxo, —C(O)C₁₋₄alkyl, —C(O)OC₁₋₄alkyl, —C(O)NH₂, —OH, —F, —Cl, —O—C₁₋₄alkyl and —CN.

Embodiment 130. The compound of any one of embodiments 1-129, or a pharmaceutically acceptable salt thereof, wherein each R^{a2} is independently selected from H and C₁₋₆ alkyl.

Embodiment 131. The compound of any one of embodiments 1-129, or a pharmaceutically acceptable salt thereof, wherein each R^{a2} is independently selected from H, —Me, —Et, —iPr, cyclopropyl, cyclobutyl, cyclopentyl, cyclohexyl, alkyl and C₂₋₆ heteroalkyl, or, when possible, two instances of R^{a2} and the atom to which they are attached are taken together to form a saturated 3-6-membered heterocycle.

Embodiment 132. The compound of any one of embodiments 1-129, or a pharmaceutically acceptable salt thereof, wherein each R^{a2} is independently selected from H and —Me.

Embodiment 133. The compound of any one of embodiments 1-132, or a pharmaceutically acceptable salt thereof, wherein each R^{b2} is independently selected from C₁₋₆ alkyl and C₃₋₉ cycloalkyl.

Embodiment 134. The compound of any one of embodiments 1-132, or a pharmaceutically acceptable salt thereof, wherein each R^{b2} is independently selected from C₁₋₆ alkyl and C₃₋₆ cycloalkyl.

Embodiment 135. The compound of any one of embodiments 1-132, or a pharmaceutically acceptable salt thereof, wherein each R^{b2} is independently selected from —Me, —Et, —iPr, cyclopropyl, cyclobutyl, cyclopentyl and cyclohexyl.

Embodiment 136. The compound of any one of embodiments 1-132, or a pharmaceutically acceptable salt thereof, wherein each R^{b2} is independently selected from —Me, —Et and ′Pr.

Embodiment 137. The compound of any one of embodiments 1-132, or a pharmaceutically acceptable salt thereof, wherein each R^{b2} is independently —Me.

Embodiment 138. The compound of any one of embodiments 1-120, or a pharmaceutically acceptable salt thereof, wherein each R² is independently selected from H, halo, OH, C₁₋₃ alkoxy, and C₁₋₃ haloalkoxy.

Embodiment 139. The compound of any one of embodiments 1-120, or a pharmaceutically acceptable salt thereof, wherein each R² is independently selected from OH, C₁₋₃ alkoxy, and C₁₋₃ haloalkoxy.

Embodiment 140. The compound of any one of embodiments 1-120, or a pharmaceutically acceptable salt thereof, wherein each R² is independently selected from halo, CN, C₁₋₃ alkyl, and C₁₋₃ haloalkyl.

Embodiment 141. The compound of any one of embodiments 1-120, or a pharmaceutically acceptable salt thereof, wherein each R² is independently selected from halo, —CN, —Me, —OMe and —OH.

Embodiment 142. The compound of any one of embodiments 1-120, or a pharmaceutically acceptable salt thereof, wherein each R² is independently selected from —F and —Me.

Embodiment 143. The compound of any one of embodiments 1-120, or a pharmaceutically acceptable salt thereof, wherein each R² is independently —F.

Embodiment 144. The compound of any one of embodiments 1-120, or a pharmaceutically acceptable salt thereof, wherein each R² is independently —Me.

Embodiment 145. The compound of any one of embodiments 1-144, or a pharmaceutically acceptable salt thereof, wherein R^{a1} is selected from ethyl, propyl, isopropyl, butyl, isobutyl, pentyl, —CH₂CF₃, —CH₂CHF₂, —CH₂CH₂CF₃, —CH₂CF₂CHF₂, —CH₂CH₂OCH₃, —CD₂CD₃, —CD(CH₃)₂, (CD₃)₂—CH—, (CD₃)₂—CD-, cyclopropyl, cyclobutyl, 3-methylcyclobutyl, 3-difluoromethylcyclobutyl, 3,3-difluorocyclobutyl, cyclopentyl, 3,3-difluorocyclopentyl, 4,4-difluorocyclohexyl, tetrahydro-1H-pyran-4-yl, tetrahydro-2H-pyran-4-yl, 2-methyltetrahydro-2H-pyran-4-yl, 3-methyltetrahydro-2H-pyran-4-yl, 2-(trifluoromethyl)tetrahydro-2H-pyran-4-yl, tetrahydrofuran-3-yl, MeO—C(O)-piperidin-4-yl, cyclopropyl-CH₂—, cyclobutyl-CH₂—, 1-trifluoromethylcyclobutyl-CH₂—, cyclopentyl-CH₂—and (tetrahydrofuran-3-yl)—CH₂—.

Embodiment 146. The compound of any one of embodiments 1-144, or a pharmaceutically acceptable salt thereof, wherein R^{a1} is selected from ethyl, isopropyl, isobutyl, tetrahydro-1H-pyran-4-yl, cyclopropyl-CH₂—, (tetrahydrofuran-3-yl)—CH₂—, —CH₂CH₂OCH₃, —CH₂CF₃ and —CH₂CHF₂.

Embodiment 147. The compound of any one of embodiments 1-144, or a pharmaceutically acceptable salt thereof, wherein R^{a1} is selected from ethyl, isopropyl, isobutyl, cyclobutyl, —CH₂CF₃ and —CH₂CHF₂.

Embodiment 148. The compound of any one of embodiments 1-144, or a pharmaceutically acceptable salt thereof, wherein R^{a1} is selected from isopropyl, cyclobutyl, —CH₂CF₃ and —CH₂CHF₂.

Embodiment 149. The compound of any one of embodiments 1-144, or a pharmaceutically acceptable salt thereof, wherein R^{a1} is C₃₋₆ cycloalkyl.

Embodiment 150. The compound of any one of embodiments 1-144, or a pharmaceutically acceptable salt thereof, wherein R^{a1} is cyclobutyl.

Embodiment 151. The compound of any one of embodiments 1-144, or a pharmaceutically acceptable salt thereof, wherein R^{a1} is C₁i- fluoroalkyl.

Embodiment 152. The compound of any one of embodiments 1-144, or a pharmaceutically acceptable salt thereof, wherein R^{a1} is —CH₂CF₃.

Embodiment 153. The compound of any one of embodiments 1-144, or a pharmaceutically acceptable salt thereof, wherein R^{a1} is C₁₋₃ alkyl.

Embodiment 154. The compound of any one of embodiments 1-144, or a pharmaceutically acceptable salt thereof, wherein R^{a1} is —′Pr.

Embodiment 155. The compound of any one of embodiments 1-154, or a pharmaceutically acceptable salt thereof, wherein R^{1A} is selected from phenyl, 4-fluorophenyl, 3-trifluoromethylphenyl, and 2-fluoro-3-cyano-phenyl.

Embodiment 156. The compound of any one of embodiments 1-154, or a pharmaceutically acceptable salt thereof, wherein R^{1A} is independently selected from H and OR^{a1} Embodiment 157. The compound of any one of embodiments 1-154, or a pharmaceutically acceptable salt thereof, wherein R^{1A} is C₁₋₃ alkyl.

Embodiment 158. The compound of any one of embodiments 1-154, or a pharmaceutically acceptable salt thereof, wherein R^{1A} is selected from propyl and isopropyl.

Embodiment 159. The compound of any one of embodiments 1-154, or a pharmaceutically acceptable salt thereof, wherein R^{1A} is selected from pyrrolidin-1-yl, 3,3-difluoropyrrolidin-1-yl, piperidin-1-yl, 3-fluoropiperidin-1-yl, 4-fluoropiperidin-1-yl, 4-methylpiperidin-lyl, (4-trifluoromethyl)piperidin-1-yl, 3,3-difluoropiperidin-1-yl, 3-(difluoromethyl)pyrrolidinyl, 2-methylpyrrolidinyl, 2-methylpiperidinyl, 3-(trifluoromethyl)piperidinyl, azabicyclo[2.2.1]heptan-7-yl, azabicyclo[2.2.1]heptan-2-yl, and (2-methoxyethyl)piperazin-1-yl.

Embodiment 160. The compound of any one of embodiments 1-154, or a pharmaceutically acceptable salt thereof, wherein R^{1A} is SR^{a1}

Embodiment 161. The compound of any one of embodiments 1-154, or a pharmaceutically acceptable salt thereof, wherein R^{1A} is NR^{a1}R^{a1}

Embodiment 162. The compound of any one of embodiments 1-154, or a pharmaceutically acceptable salt thereof, wherein R^{1A} is OR^{a1} Embodiment 163. The compound of any one of embodiments 1-90, or a pharmaceutically acceptable salt thereof, wherein R^{1A} is selected from ethoxy, propoxy, isopropoxy, butoxy, isobutoxy, pentoxy, —OCH₂CF₃, —OCH₂CHF₂, —OCH₂CH₂CF₃, —OCH₂CF₂CHF₂, —OCH₂CH₂OCH₃, —OCD₂CD₃, —OCD(CH₃)₂, —OCH(CD₃)₂, —OCD(CD₃)₂, cyclopropoxy, cyclobutoxy, 3-methylcyclobutoxy, 3-difluoromethylcyclobutoxy, 3,3-difluorocyclobutoxy, cyclopentoxy, 3,3-difluorocyclopentoxy, 4,4-difluorocyclohexyloxy, tetrahydro-1H-pyran-4-oxy, tetrahydro-2H-pyran-4-oxy, 2-methyltetrahydro-2H-pyran-4-oxy, 3-methyltetrahydro-2H-pyran-4-oxy, 2-(trifluoromethyl)tetrahydro-2H-pyran-4-oxy, tetrahydrofuran-3-oxy, MeO—C(O)-piperidin-4-oxy, cyclopropyl-CH₂—O—, cyclobutyl-CH₂—O—, 1-trifluoromethylcyclobutyl-CH₂—O—, cyclopentyl-CH₂—O—and (tetrahydrofuran-3-yl)—CH₂—O

Embodiment 164. The compound of any one of embodiments 1-90, or a pharmaceutically acceptable salt thereof, wherein R^{1A} is selected from ethoxy, isopropoxy, isobutoxy, tetrahydro-1H-pyran-4-oxy, cyclopropyl-CH₂—O—, (tetrahydrofuran-3-yl)—CH₂—O—, —OCH₂CH₂OCH₃, —OCH₂CF₃ and —OCH₂CHF₂.

Embodiment 165. The compound of any one of embodiments 1-90, or a pharmaceutically acceptable salt thereof, wherein R^{1A} is selected from ethoxy, isopropoxy, isobutoxy, cyclobutoxy, —OCH₂CF₃ and —OCH₂CHF₂.

Embodiment 166. The compound of any one of embodiments 1-90, or a pharmaceutically acceptable salt thereof, wherein R^{1A} is selected from isopropoxy, cyclobutoxy, —OCH₂CF₃ and —OCH₂CHF₂.

Embodiment 167. The compound of any one of embodiments 1-90, or a pharmaceutically acceptable salt thereof, wherein R^{1A} is C₃₋₆ cycloalkoxy.

Embodiment 168. The compound of any one of embodiments 1-90, or a pharmaceutically acceptable salt thereof, wherein R^{1A} is cyclobutoxy.

Embodiment 169. The compound of any one of embodiments 1-90, or a pharmaceutically acceptable salt thereof, wherein R^{1A} is C₁₋₆ fluoroalkoxy.

Embodiment 170. The compound of any one of embodiments 1-90, or a pharmaceutically acceptable salt thereof, wherein R^{1A} is —OCH₂CF₃. Embodiment 171. The compound of any one of embodiments 1-90, or a pharmaceutically acceptable salt thereof, wherein R^{1A} is C₁₋₃ alkoxy.

Embodiment 172. The compound of any one of embodiments 1-90, or a pharmaceutically acceptable salt thereof, wherein R^{1A} is —O′Pr.

Embodiment 173. A compound of Formula I:

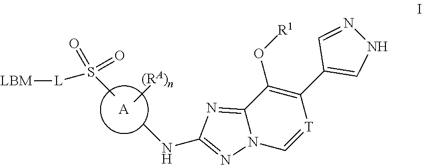

or a pharmaceutically acceptable salt thereof, wherein:
Ring A is selected from

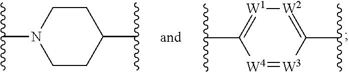

each W¹, W², W³ and W⁴ is independently selected from CH and N, provided that no more than 2 of W¹, W², W³ and W⁴ are N;
T is CH or N;
R¹ is independently selected from H, C₁₋₆ alkyl, C₁₋₆ haloalkyl, C₃₋₁₀ cycloalkyl, 4-10 membered heterocyclyl, C₃₋₁₀ cycloalkyl-C₁₋₄ alkyl- and 4-10 membered heterocyclyl-C₁₋₄ alkyl-, wherein said C₁₋₆ alkyl, C₁₋₆ haloalkyl, C₃₋₁₀ cycloalkyl, 4-10 membered heterocyclyl, C₃₋₁₀ cycloalkyl-C₁₋₄ alkyl- and 4-10 membered heterocyclyl-C₁₋₄ alkyl-, are each substituted with 0, 1, 2, 3, or 4 independently selected R² substituents;
each R² is independently selected from D, halo, CN, C₁₋₆ alkyl, C₁₋₆ haloalkyl, C₃₋₇ cycloalkyl, OR^{a2}, C(O)R^{b2}, C(O)NR^{a2}R^{a2}, C(O)OR^{a2}, OC(O)R^{b2}, OC(O)NR^{a2}R^{a2}, NR^{a2}R^{a2}, NR^{a2}C(O)R^{b2}, NR^{a2}C(O)OR^{a2}, NR^{a2}C(O)NR^{a2}R^{a2}, NR^{a2}S(O)NR^{a2}R^{a2}, NR^{a2}S(O)R^{b2}, NR^{a2}S(O)₂R^{b2}, NR^{a2}S(O)₂NR^{a2}R^{a2}, S(O)R^{b2}, S(O)NR^{a2}R^{a2}, S(O)₂R^{b2}, S(O)₂NR^{a2}R^{a2}, SF₅, wherein said C₁₋₆ alkyl, C₁₋₆ haloalkyl, C₃₋₇ cycloalkyl and 4-7 membered heterocyclyl are each optionally substituted with 1, 2, 3, or 4 substituents independently selected from C₁₋₄ alkyl, C₃₋₇ cycloalkyl, cyclopropyl, oxo, —C(O)C₁₋₄alkyl, —C(O)OC₁₋₄alkyl, —C(O)NH₂, —OH, —F, —Cl, —O—C₁₋₄alkyl and —CN;
each R^{a2} is independently selected from H, C₁₋₆ alkyl, C₁₋₆ hydroxyalkyl, C₃₋₉ cycloalkyl and C₂₋₆ heteroalkyl, or, when possible, two instances of R^{a2} and the atom to which they are attached are taken together to form a saturated 3-7-membered heterocycle;
each R^{b2} is independently selected from C₁₋₆ alkyl, C₁₋₆ hydroxyalkyl, C₃₋₉ cycloalkyl and C₂₋₆ heteroalkyl;
L is a covalent bond or a bivalent, saturated or unsaturated, straight or branched C₁₋₅₀ hydrocarbon chain, wherein 0-6 methylene units of L are independently replaced by —Cy—, —CH(R)—, —C(R)₂—, —O—, —NR—, —S—, —OC(═O)—, —C(═O)O—, —C(═O)—, —S(═O)—, —S(═O)₂—, —NRS(═O)₂—, —S(═O)₂NR—, —NRC(═O)—, —C(═O)NR—, —OC(═O)NR— or —NRC(═O)O—, wherein:
each —Cy— is independently a bivalent ring selected from phenylene, an 8-10 membered bicyclic arylene, a 4-7 membered monocyclic carbocyclylene, a 5-11 membered spiro carbocyclylene, a 4-10 membered bicyclic carbocyclylene, a 5-10 membered bridged carbocyclylene, a 4-7 membered monocyclic heterocyclylene having 1-2 heteroatoms independently selected from nitrogen, oxygen, and sulfur, a 5-11 membered spiro heterocyclylene having 1-2 heteroatoms independently selected from nitrogen, oxygen, and sulfur, an 4-10 membered bicyclic heterocyclylene having 1-2 heteroatoms independently selected from nitrogen, oxygen, and sulfur, a 5-10 membered bridged bicyclic saturated or partially unsaturated heterocyclylene having 1-2 heteroatoms independently selected from nitrogen, oxygen, and sulfur, a 5-6 membered heteroarylene having 1-4 heteroatoms independently selected from nitrogen, oxygen, and sulfur, or an 8-10 membered bicyclic heteroarylene having 1-5 heteroatoms independently selected from nitrogen, oxygen, or sulfur, wherein each phenylene, arylene, carbocyclylene, heterocyclylene and heteroarylene is substituted with 0, 1, 2, 3, or 4 instances of R^{C};
LBM is selected from:

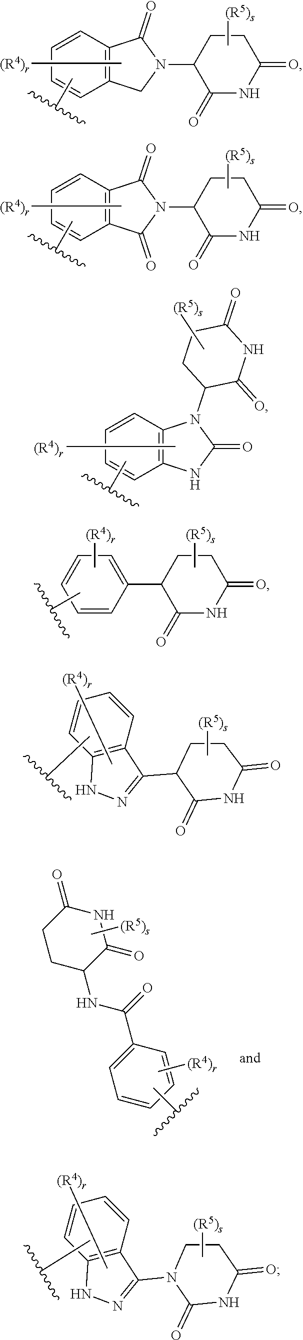

each instance of R^{A} is independently selected from —D, halogen, —C₁₋₆ alkyl, —OH and —OC₁₋₄ alkyl, wherein each —C₁₋₆ alkyl is substituted with 0, 1, 2 or 3 groups independently selected from —D, —F, —OH and —OC₁₋₄ alkyl or two R^{A} groups on adjacent atoms of Ring A, together with the ring atoms to which they are attached, form Ring D, wherein Ring D is selected from C₃₋₆ cycloalkyl, 4-6 membered heterocyclyl, phenyl, and 5-6 membered heteroaryl, each of which is substituted with 0, 1, 2, 3, or 4 substituents independently selected from —D, halo, —OH, —C₁₋₄ alkyl and —OC₁₋₄ alkyl;
each instance of R^{C} is independently selected from —D, halogen, —OH, and —C₁₋₆ alkyl;
each instance of R⁴ is independently selected from —D, halogen, —OH, and —C₁₋₆ alkyl;
each instance of R⁵ is independently selected from —D, halogen, —OH, and —C₁₋₆ alkyl;
each instance of^{R} is independently selected from hydrogen and —C₁₋₆ alkyl;
n is 0, 1, 2, 3, or 4;
r is 0, 1, 2, 3, or 4; and
s is 0, 1, 2, 3, or 4.

Embodiment 174. The compound of embodiment 173, wherein the compound is of formula

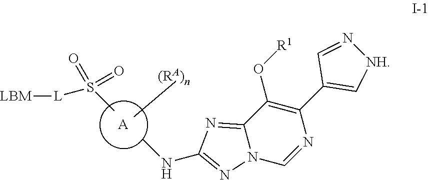

Embodiment 175. The compound of embodiment 173, wherein the compound is of formula

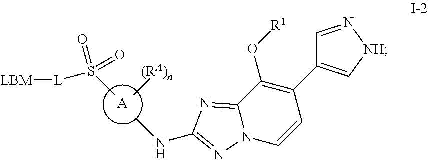

Embodiment 176. The compound of any one of embodiments 173-175, or a pharmaceutically acceptable salt thereof, wherein Ring A is selected from

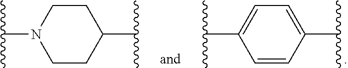 [SMILES: *C1CCN(*)CC1 ; *c1ccc(*)cc1]

Embodiment 177. The compound of any one of embodiments 173-175, or a pharmaceutically acceptable salt thereof, wherein Ring A is

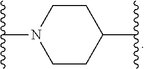 [SMILES: *C1CCN(*)CC1]

Embodiment 178. The compound of any one of embodiments 173-175, or a pharmaceutically acceptable salt thereof, wherein Ring A is

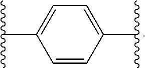 [SMILES: *c1ccc(*)cc1]

Embodiment 179. The compound of embodiment 173, or a pharmaceutically acceptable salt thereof, wherein the compound is a compound of Formula II

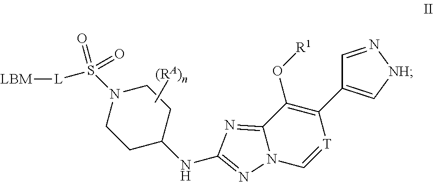

Embodiment 180. The compound of embodiment 173, or a pharmaceutically acceptable salt thereof, wherein the compound is a compound of Formula II-1

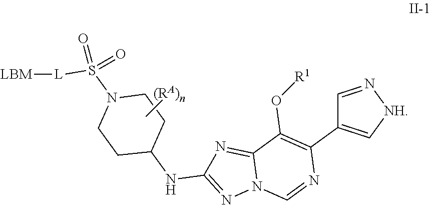

Embodiment 181. The compound of embodiment 173, or a pharmaceutically acceptable salt thereof, wherein the compound is a compound of Formula II-2

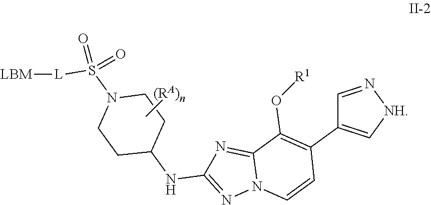

Embodiment 182. The compound of embodiment 173, or a pharmaceutically acceptable salt thereof, wherein the compound is a compound of Formula III

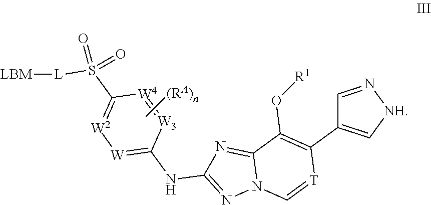

Embodiment 183. The compound of embodiment 173, or a pharmaceutically acceptable salt thereof, wherein the compound is a compound of Formula III-1

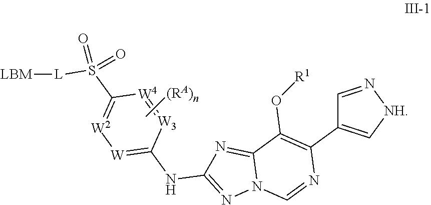

Embodiment 184. The compound of embodiment 173, or a pharmaceutically acceptable salt thereof, wherein the compound is a compound of Formula III-2

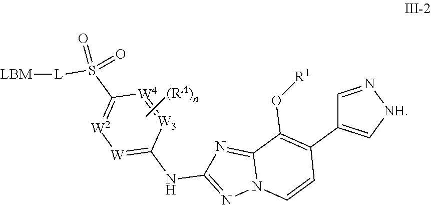

Embodiment 185. The compound of embodiment 173, or a pharmaceutically acceptable salt thereof, wherein the compound is a compound of Formula III-a

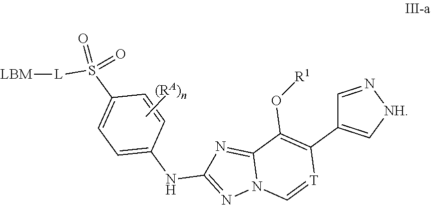

Embodiment 186. The compound of embodiment 173, or a pharmaceutically acceptable salt thereof, wherein the compound is a compound of Formula III-a-I

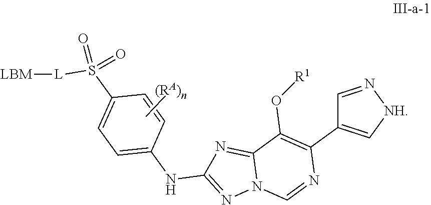

Embodiment 187. The compound of embodiment 173, or a pharmaceutically acceptable salt thereof, wherein the compound is a compound of Formula III-a-2

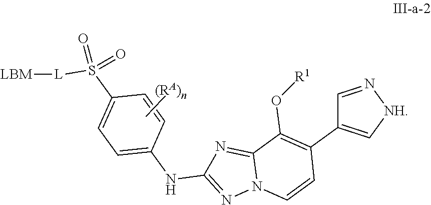

Embodiment 188. The compound of any one of embodiments 173-187, or a pharmaceutically acceptable salt thereof, wherein R¹ is selected from C₁₋₆ alkyl, C₁₋₆ haloalkyl and C₃₋₆ cycloalkyl, wherein said C₁₋₆ alkyl, C₁₋₆ haloalkyl and C₃₋₆ cycloalkyl are each substituted with 0, 1 or 2 independently selected R² substituents.

Embodiment 189. The compound of any one of embodiments 173-187, or a pharmaceutically acceptable salt thereof, wherein R¹ is selected from C₁₋₄ alkyl, C₁₋₃ fluoroalkyl and C₃₋₆ cycloalkyl, wherein said C₁₋₄ alkyl, C₁₋₃ fluoroalkyl and C₃₋₆ cycloalkyl are each substituted with 0, 1 or 2 independently selected R² substituents.

Embodiment 190. The compound of any one of embodiments 173-187 or a pharmaceutically acceptable salt thereof, wherein R¹ is selected from —Me, —Et, —Pr, —′Pr, iso-butyl, sec-butyl, ′butyl, cyclopropyl, cyclobutyl, cyclopentyl, —CF₃, —CHF₂, CH₂CF₃, cyclopropyl, cyclobutyl, cyclopentyl and cyclohexyl, each substituted with 0, 1 or 2 independently selected R² substituents.

Embodiment 191. The compound of any one of embodiments 173-190, or a pharmaceutically acceptable salt thereof, wherein each R² is independently selected from halo, CN, C₁₋₄ alkyl, C₁₋₄ haloalkyl, OH, C₁₋₃ alkoxy, C₁₋₃ haloalkoxy, amino, C₁₋₃ alkylamino, di(C₁₋₃ alkyl)amino, cyano-C₁₋₄ alkyl, HO—C₁₋₄ alkyl, C₁₋₃ alkoxy-C₁₋₄ alkyl, and C₃-4 cycloalkyl.

Embodiment 192. The compound of any one of embodiments 173-190, or a pharmaceutically acceptable salt thereof, wherein each R² is independently selected from D, halo, CN, C₁₋₆ alkyl, C₁₋₆ haloalkyl, C₃₋₄ cycloalkyl, OR^{a2}, C(O)R^{b2}, C(O)NR^{a2}R^{a2}, C(O)OR^{a2}, OC(O)R^{b2}, OC(O)NR^{a2}R^{a2}, NR^{a2}R^{a2}, NR^{a2}C(O)R^{b2}, NR^{a2}C(O)OR^{a2}, NR^{a2}C(O)NR^{a2}R^{a2}, NR^{a2}S(O)₂R^{b2}, NR^{a2}S(O)₂NR^{a2}R^{a2}, S(O)₂R^{b2}, and S(O)₂NR^{a2}R^{a2}, wherein said C₁₋₆ alkyl, C₁₋₆ 20 haloalkyl, and C₃-4 cycloalkyl are each substituted with 0, 1, 2, 3, or 4 substituents independently selected from C₁₋₄ alkyl, C₃₋₇ cycloalkyl, cyclopropyl, oxo, —C(O)C₁₋₄alkyl, —C(O)OC₁₋₄alkyl, —C(O)NH₂, —OH, —F, —Cl, —O—C₁₋₄alkyl and —CN.

Embodiment 193. The compound of any one of embodiments 173-190, or a pharmaceutically acceptable salt thereof, wherein each R² is independently selected from D, halo, CN, C₁₋₄ alkyl, C₁₋₄ haloalkyl, OR^{a2}, and C(O)OR^{a2}, wherein said C₁₋₄ alkyl and C₁₋₄ haloalkyl are each substituted with 0, 1, 2, 3, or 4 substituents independently selected from C₁₋₄ alkyl, C₃₋₇ cycloalkyl, cyclopropyl, oxo, —C(O)C₁₋₄alkyl, —C(O)OC₁₋₄alkyl, —C(O)NH₂, —OH, —F, —Cl, —O—C₁₋₄alkyl and —CN.

Embodiment 194. The compound of any one of embodiments 173-193, or a 30 pharmaceutically acceptable salt thereof, wherein each R^{a2} is independently selected from H and C₁₋₆ alkyl.

Embodiment 195. The compound of any one of embodiments 173-193, or a pharmaceutically acceptable salt thereof, wherein each R^{a2} is independently selected from H, —Me, —Et, —iPr, cyclopropyl, cyclobutyl, cyclopentyl, cyclohexyl, alkyl and C₂₋₆ heteroalkyl, or, when possible, two instances of R^{a2} and the atom to which they are attached are taken together to form a saturated 3-6-membered heterocycle.

Embodiment 196. The compound of any one of embodiments 173-193, or a 5 pharmaceutically acceptable salt thereof, wherein each R^{a2} is independently selected from H and —Me.

Embodiment 197. The compound of any one of embodiments 173-196, or a pharmaceutically acceptable salt thereof, wherein each R^{b2} is independently selected from C₁₋₆ alkyl and C₃₋₉ cycloalkyl.

Embodiment 198. The compound of any one of embodiments 173-193, or a pharmaceutically acceptable salt thereof, wherein each R^{b2} is independently selected from C₁-6 alkyl and C₃₋₆ cycloalkyl.

Embodiment 199. The compound of any one of embodiments 173-193, or a pharmaceutically acceptable salt thereof, wherein each R^{b2} is independently selected from —Me, —Et, —iPr, cyclopropyl, cyclobutyl, cyclopentyl, cyclohexyl, alkyl and C₂₋₆ heteroalkyl, or, when possible, two instances of R^{a2} and the atom to which they are attached are taken together to form a saturated 3-6-membered heterocycle.

Embodiment 200. The compound of any one of embodiments 173-193, or a pharmaceutically acceptable salt thereof, wherein each R^{b2} is independently selected from —Me, —Et and ′Pr,

Embodiment 201. The compound of any one of embodiments 173-193, or a pharmaceutically acceptable salt thereof, wherein each R^{b2} is independently —Me.

Embodiment 202. The compound of any one of embodiments 173-190, or a pharmaceutically acceptable salt thereof, wherein each R² is independently selected from H, halo, OH, C₁₋₃ alkoxy, and C₁₋₃ haloalkoxy.

Embodiment 203. The compound of any one of embodiments 173-190, or a pharmaceutically acceptable salt thereof, wherein each R² is independently selected from OH, C₁₋₃ alkoxy, and C₁₋₃ haloalkoxy.

Embodiment 204. The compound of any one of embodiments 173-190, or a pharmaceutically acceptable salt thereof, wherein each R² is independently selected from halo, CN, C₁₋₃ alkyl, and C₁₋₃ haloalkyl.

Embodiment 205. The compound of any one of embodiments 173-190, or a pharmaceutically acceptable salt thereof, wherein each R² is independently selected from halo, —CN, —Me, —OMe and —OH.

Embodiment 206. The compound of any one of embodiments 173-190, or a pharmaceutically acceptable salt thereof, wherein each R² is independently selected from —F and —Me.

Embodiment 207. The compound of any one of embodiments 173-190, or a pharmaceutically acceptable salt thereof, wherein each R² is independently —F.

Embodiment 208. The compound of any one of embodiments 173-190, or a pharmaceutically acceptable salt thereof, wherein each R² is independently —Me.

Embodiment 209. The compound of any one of embodiments 173-187, or a pharmaceutically acceptable salt thereof, wherein R¹ is selected from ethyl, propyl, isopropyl, butyl, isobutyl, pentyl, —CH₂CF₃, —CH₂CHF₂, —CH₂CH₂CF₃, —CH₂CF₂CHF₂, —CH₂CH₂OCH₃, —CD₂CD₃, —CD(CH₃)₂, (CD₃)₂—CH—, (CD₃)₂—CD-, cyclopropyl, cyclobutyl, 3-methylcyclobutyl, 3-difluoromethylcyclobutyl, 3,3-difluorocyclobutyl, cyclopentyl, 3,3-difluorocyclopentyl, 4,4-difluorocyclohexyl, tetrahydro-1H-pyran-4-yl, tetrahydro-2H-pyran-4-yl, 2-methyltetrahydro-2H-pyran-4-yl, 3-methyltetrahydro-2H-pyran-4- yl, 2-(trifluoromethyl)tetrahydro-2H-pyran-4-yl, tetrahydrofuran-3-yl, MeO—C(O)-piperidin-4-yl, cyclopropyl-CH₂—, cyclobutyl-CH₂—, 1-trifluoromethylcyclobutyl-CH₂—, cyclopentyl-CH₂—, and (tetrahydrofuran-3-yl)—CH₂—.

Embodiment 210. The compound of any one of embodiments 173-187, or a pharmaceutically acceptable salt thereof, wherein R¹ is selected from ethyl, isopropyl, isobutyl, tetrahydro-1H-pyran-4-yl, cyclopropyl-CH₂—, (tetrahydrofuran-3-yl)—CH₂—, —CH₂CH₂OCH₃, —CH₂CF₃, and —CH₂CHF₂.

Embodiment 211. The compound of any one of embodiments 173-187, or a pharmaceutically acceptable salt thereof, wherein R¹ is selected from ethyl, isopropyl, isobutyl, cyclobutyl, —CH₂CF₃, and —CH₂CHF₂.

Embodiment 212. The compound of any one of embodiments 173-187, or a pharmaceutically acceptable salt thereof, wherein R¹ is selected from isopropyl, cyclobutyl, —CH₂CF₃, and —CH₂CHF₂.

Embodiment 213. The compound of any one of embodiments 173-187, or a pharmaceutically acceptable salt thereof, wherein R¹ is C₄₋₇ heterocyclyl.

Embodiment 214. The compound of any one of embodiments 173-187, or a pharmaceutically acceptable salt thereof, wherein R¹ is C₃₋₆ cycloalkyl.

Embodiment 215. The compound of any one of embodiments 173-187, or a pharmaceutically acceptable salt thereof, wherein R¹ is cyclobutyl.

Embodiment 216. The compound of any one of embodiments 173-187, or a pharmaceutically acceptable salt thereof, wherein R¹ is C₁₋₆ fluoroalkyl.

Embodiment 217. The compound of any one of embodiments 173-187, or a pharmaceutically acceptable salt thereof, wherein R¹ is —CH₂CF₃.

Embodiment 218. The compound of any one of embodiments 173-187, or a pharmaceutically acceptable salt thereof, wherein R¹ is C₁₋₃ alkyl.

Embodiment 219. The compound of any one of embodiments 173-187, or a pharmaceutically acceptable salt thereof, wherein R¹ is —′Pr.

Embodiment 220. The compound of any one of embodiments 1-219, or a pharmaceutically acceptable salt thereof, wherein each R^{A} is independently selected from —D, halogen, —C₁₋₆ alkyl, —OH and —OC₁₋₄ alkyl, wherein each —C₁₋₆ alkyl is substituted with 0, 1, 2 or 3 groups independently selected from —D, —F, —OH and —OC₁₋₄ alkyl or two R^{A} are taken together with the atoms to which they are attached to form an aryl or heteroaryl.

Embodiment 221. The compound of any one of embodiments 1-219, or a pharmaceutically acceptable salt thereof, wherein each R^{A} is independently selected from —D, halogen, —C₁₋₆ alkyl, —OH and —OC₁₋₄ alkyl, wherein each —C₁₋₆ alkyl is substituted with 0, 1, 2 or 3 groups independently selected from —D, —F, —OH and —OC₁₋₄ alkyl.

Embodiment 222. The compound of any one of embodiments 1-219, or a pharmaceutically acceptable salt thereof, wherein two R^{A} are taken together with the atoms to which they are attached to form an aryl or heteroaryl.

Embodiment 223. The compound of any one of embodiments 1-219, or a pharmaceutically acceptable salt thereof, wherein each R^{A} is independently selected from —D, —Me, —Et, —iPr, —CF₃, —CHF₂, —F, —Cl, —OH, —OMe, OCF₃ and —OCHF₂.

Embodiment 224. The compound of any one of embodiments 1-219, or a pharmaceutically acceptable salt thereof, wherein each R^{A} is independently selected from —D, —F, —Cl, —Me and —OH.

Embodiment 225. The compound of any one of embodiments 1-219, or a pharmaceutically acceptable salt thereof, wherein each R^{A} is independently selected from —D, —F and —Me.

Embodiment 226. The compound of any one of embodiments 1-219, or a pharmaceutically acceptable salt thereof, wherein each R^{A} is independently selected from —F and —Me or two R^{A} are taken together with the atoms to which they are attached to form an aryl or heteroaryl.

Embodiment 227. The compound of any one of embodiments 1-219, or a pharmaceutically acceptable salt thereof, wherein each R^{A} is independently selected from —F and —Me or two R^{A} are taken together with the atoms to which they are attached to form a pyridinyl or furanyl.

Embodiment 228. The compound of any one of embodiments 1-219, or a pharmaceutically acceptable salt thereof, wherein each R^{A} is independently selected from —F and —Me.

Embodiment 229. The compound of any one of embodiments 1-219, or a pharmaceutically acceptable salt thereof, wherein each R^{A} is independently —D.

Embodiment 230. The compound of any one of embodiments 1-219, or a pharmaceutically acceptable salt thereof, wherein each R^{A} is independently —F.

Embodiment 231. The compound of any one of embodiments 1-219, or a pharmaceutically acceptable salt thereof, wherein each R^{A} is independently —Cl.

Embodiment 232. The compound of any one of embodiments 1-219, or a pharmaceutically acceptable salt thereof, wherein each R^{A} is independently —Me.

Embodiment 233. The compound of any one of embodiments 1-219, or a pharmaceutically acceptable salt thereof, wherein each R^{A} is independently —OH.

Embodiment 234. The compound of any one of embodiments 1-219, or a pharmaceutically acceptable salt thereof, wherein n is 0, 1 or 2.

Embodiment 235. The compound of any one of embodiments 1-219, or a pharmaceutically acceptable salt thereof, wherein n is 0 or 1.

Embodiment 236. The compound of any one of embodiments 1-219, or a pharmaceutically acceptable salt thereof, wherein n is 0.

Embodiment 237. The compound of any one of embodiments 1-219, or a pharmaceutically acceptable salt thereof, wherein n is 1.

Embodiment 238. The compound of any one of embodiments 1-219, or a pharmaceutically acceptable salt thereof, wherein n is 2.

Embodiment 239. The compound of any one of embodiments 1-219, or a pharmaceutically acceptable salt thereof, wherein the moiety represented by

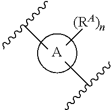

is selected from

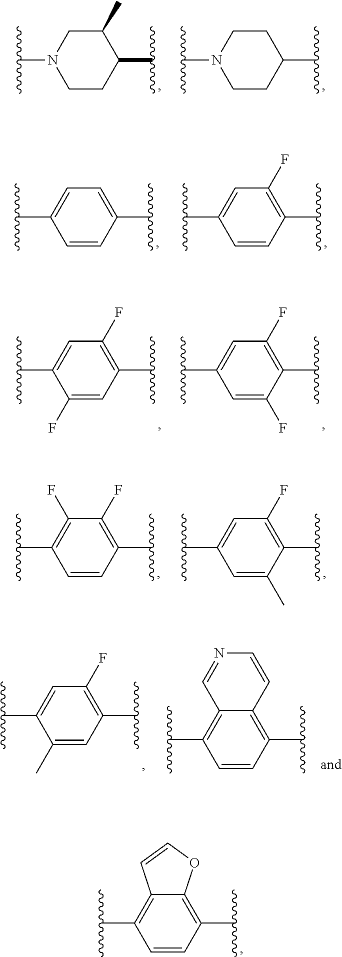

wherein the left attachment point connects to the —X— group of Formula A-I or the —S(O)₂— group of formula I and the right attachment point connects to the —NH— group of Formula A-I and formula I.

Embodiment 240. The compound of any one of embodiments 1-219, or a pharmaceutically acceptable salt thereof, wherein the moiety represented by

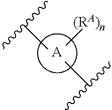

is selected from

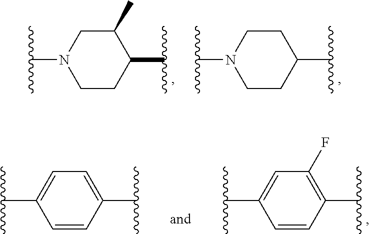

wherein the left attachment point connects to the —X— group of Formula A-I or the —S(O)₂— group of formula I and the right attachment point connects to the —NH— group of Formula A-I and formula I.

Embodiment 241. The compound of any one of embodiments 1-219, or a pharmaceutically (R^{A})q acceptable salt thereof, wherein the moiety represented by

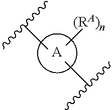

selected from

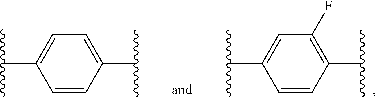 [SMILES: *c1ccc(*)cc1 ; *c1ccc(*)c(F)c1]

wherein the left attachment point connects to the —X— group of Formula A-I or the —S(O)₂— group of formula I and the right attachment point connects to the —NH— group of Formula A-I and formula I.

Embodiment 242. The compound of any one of embodiments 1-219, or a pharmaceutically acceptable salt thereof, wherein the moiety represented by

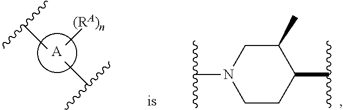

wherein the left attachment point connects to the —X— group of Formula A-I or the —S(O)₂— group of formula I and the right attachment point connects to the —NH— group of Formula A-I and formula I.

Embodiment 243. The compound of any one of embodiments 1-219, or a pharmaceutically acceptable salt thereof, wherein the moiety represented by

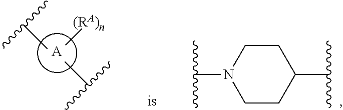

wherein the left attachment point connects to the —X— group of Formula A-I or the —S(O)₂— group of formula I and the right attachment point connects to the —NH— group of Formula A-I and formula I.

Embodiment 244. The compound of any one of embodiments 1-219, or a pharmaceutically acceptable salt thereof, wherein the moiety represented by

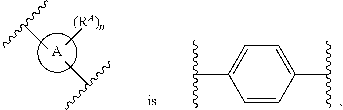

wherein the left attachment point connects to the —X— group of Formula A-I or the —S(O)₂— group of formula I and the right attachment point connects to the —NH— group of Formula A-I and formula I.

Embodiment 245. The compound of any one of embodiments 1-219, or a pharmaceutically acceptable salt thereof, wherein the moiety represented by

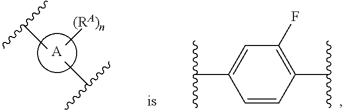

wherein the left attachment point connects to the —X— group of Formula A-I or the —S(O)₂— group of formula I and the right attachment point connects to the —NH— group of Formula A-I and formula I.

Embodiment 246. The compound of any one of embodiments 1-219, or a pharmaceutically acceptable salt thereof, wherein the moiety represented by

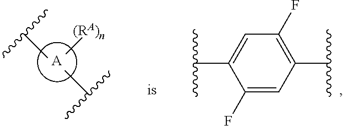

wherein the left attachment point connects to the —X— group of Formula A-I or the —S(O)₂— group of formula I and the right attachment point connects to the —NH— group of Formula A-I and formula I.

Embodiment 247. The compound of any one of embodiments 1-219, or a pharmaceutically acceptable salt thereof, wherein the moiety represented by

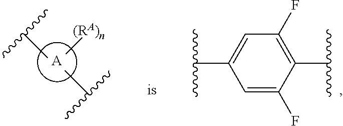

wherein the left attachment point connects to the —X— group of Formula A-I or the —S(O)₂— group of formula I and the right attachment point connects to the —NH— group of Formula A-I and formula I.

Embodiment 248. The compound of any one of embodiments 1-219, or a pharmaceutically acceptable salt thereof, wherein the moiety represented by

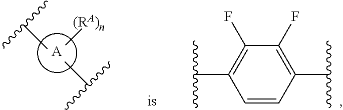

wherein the left attachment point connects to the —X— group of Formula A-I or the —S(O)₂— group of formula I and the right attachment point connects to the —NH— group of Formula A-I and formula I.

Embodiment 249. The compound of any one of embodiments 1-219, or a pharmaceutically acceptable salt thereof, wherein the moiety represented by

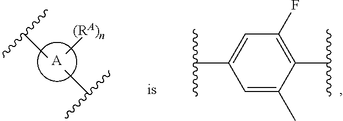

wherein the left attachment point connects to the —X— group of Formula A-I or the —S(O)₂— group of formula I and the right attachment point connects to the —NH— group of Formula A-I and formula I.

Embodiment 250. The compound of any one of embodiments 1-219, or a pharmaceutically acceptable salt thereof, wherein the moiety represented by

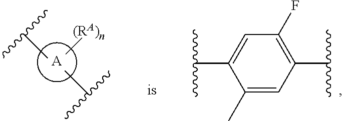

wherein the left attachment point connects to the —X— group of Formula A-I or the —S(O)₂— group of formula I and the right attachment point connects to the —NH— group of Formula A-I and formula I.

Embodiment 251. The compound of any one of embodiments 1-219, or a pharmaceutically acceptable salt thereof, wherein the moiety represented by

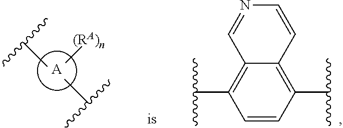

wherein the left attachment point connects to the —X— group of Formula A-I or the —S(O)₂— group of formula I and the right attachment point connects to the —NH— group of Formula A-I and formula I.

Embodiment 252. The compound of any one of embodiments 1-219, or a pharmaceutically acceptable salt thereof, wherein the moiety represented by

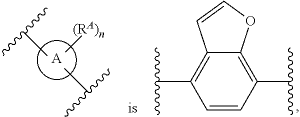

wherein the left attachment point connects to the —X— group of Formula A-I or the —S(O)₂— group of formula I and the right attachment point connects to the —NH— group of Formula A-I and formula I.

Embodiment 253. The compound of any one of embodiments 1-233, or a pharmaceutically acceptable salt thereof, wherein the compound is of Formula II-a.

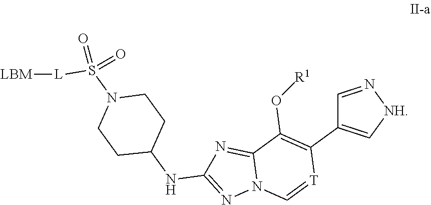

Embodiment 254. The compound of any one of embodiments 1-233, or a pharmaceutically acceptable salt thereof, wherein the compound is of Formula II-a-1.

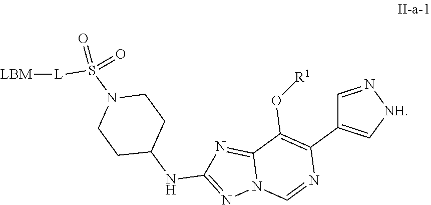

Embodiment 255. The compound of any one of embodiments 1-233, or a pharmaceutically acceptable salt thereof, wherein the compound is of Formula II-a-2.

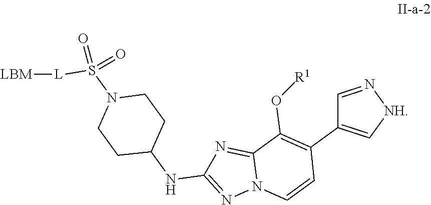

Embodiment 256. The compound of any one of embodiments 1-233 or a pharmaceutically acceptable salt thereof, wherein the compound is of Formula II-b.

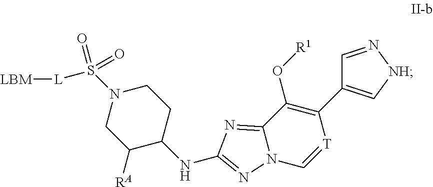

Embodiment 257. The compound of any one of embodiments 1-233, or a pharmaceutically acceptable salt thereof, wherein the compound is of Formula II-b-1.

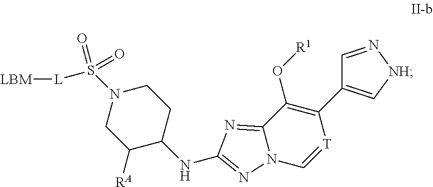

Embodiment 258. The compound of any one of embodiments 1-233, or a pharmaceutically acceptable salt thereof, wherein the compound is of Formula II-b-2.

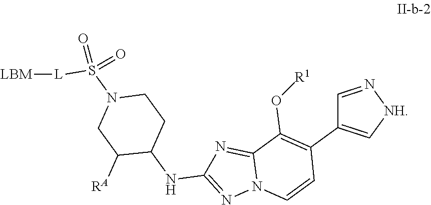

Embodiment 259. The compound of any one of embodiments 256-258, wherein R^{A} and the —NH— group attached to the piperidine ring are in a cis configuration.

Embodiment 260. The compound of any one of embodiments 256-258, wherein R^{A} and the —NH— group attached to the piperidine ring are in a trans configuration.

Embodiment 261. The compound of any one of embodiments 256-258, wherein R^{A} and the —NH— group attached to the piperidine ring are in an R, S configuration.

Embodiment 262. The compound of any one of embodiments 256-258, wherein R^{A} and the —NH— group attached to the piperidine ring are in an R, R configuration.

Embodiment 263. The compound of any one of embodiments 256-258, wherein R^{A} and the —NH— group attached to the piperidine ring are in an S, S configuration.

Embodiment 264. The compound of any one of embodiments 256-258, wherein R^{A} and the —NH— group attached to the piperidine ring are in an S, R configuration.

Embodiment 265. The compound of any one of embodiments 1-233, or a pharmaceutically acceptable salt thereof, wherein the compound is of Formula III-b

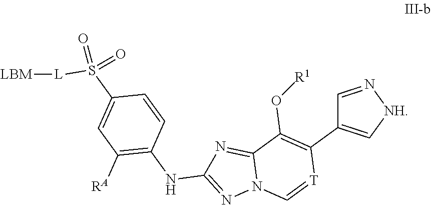

Embodiment 266. The compound of any one of embodiments 1-233, or a pharmaceutically acceptable salt thereof, wherein the compound is of Formula III-b-1

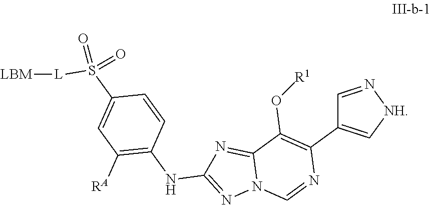

Embodiment 267. The compound of any one of embodiments 1-233, or a pharmaceutically acceptable salt thereof, wherein the compound is of Formula III-b-2

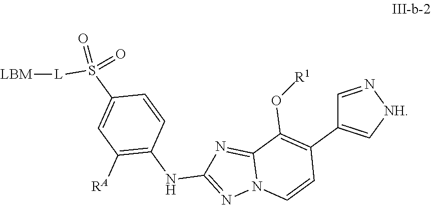

Embodiment 268. The compound of any one of embodiments 1-233, or a pharmaceutically acceptable salt thereof, wherein the compound is of Formula III-c:

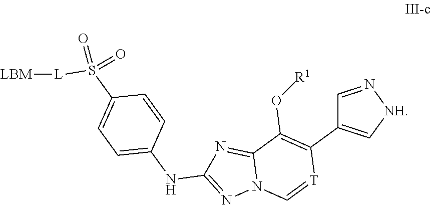

Embodiment 269. The compound of any one of embodiments 1-233, or a pharmaceutically acceptable salt thereof, wherein the compound is of Formula III-c-1:

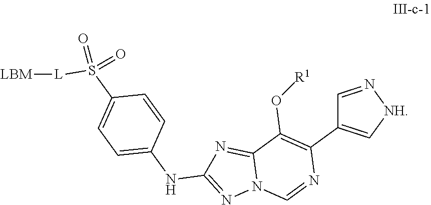

Embodiment 270. The compound of any one of embodiments 1-233, or a pharmaceutically acceptable salt thereof, wherein the compound is of Formula III-c-2:

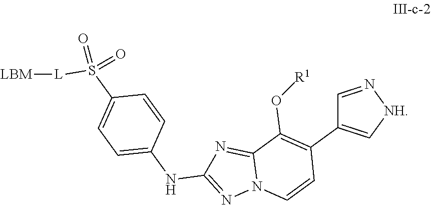

Embodiment 271. The compound of any one of embodiments 1-233, or a pharmaceutically acceptable salt thereof, wherein the compound is of Formula IV.

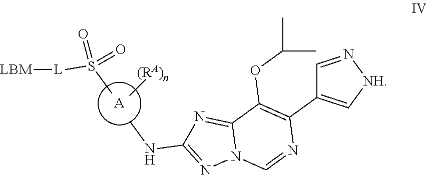

Embodiment 272. The compound of any one of embodiments 1-233, or a pharmaceutically acceptable salt thereof, wherein the compound is of Formula IV-a.

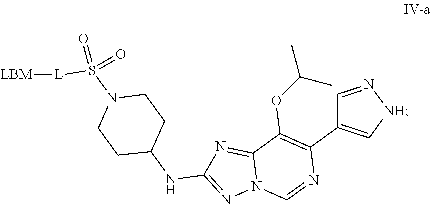

Embodiment 273. The compound of any one of embodiments 1-233, or a pharmaceutically acceptable salt thereof, wherein the compound is of Formula IV-b.

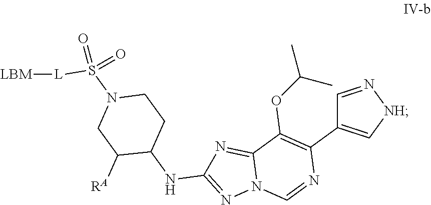

Embodiment 274. The compound of any one of embodiments 1-233, or a pharmaceutically acceptable salt thereof, wherein the compound is of Formula IV-c.

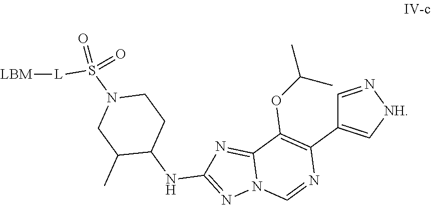

Embodiment 275. The compound of any one of embodiments 1-233 or a pharmaceutically acceptable salt thereof, wherein the compound is of Formula IV-d.

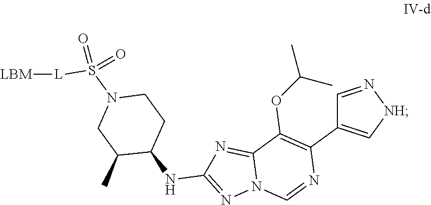

Embodiment 276. The compound of any one of embodiments 1-233, or a pharmaceutically acceptable salt thereof, wherein the compound is of Formula IV-e

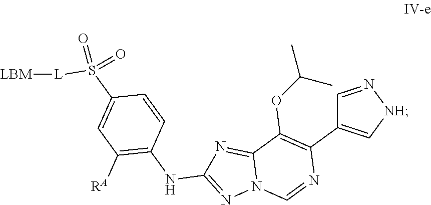

Embodiment 277. The compound of any one of embodiments 1-233, or a pharmaceutically acceptable salt thereof, wherein the compound is of Formula IV-f

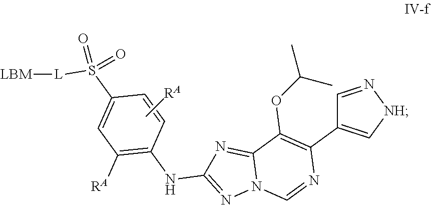

Embodiment 278. The compound of any one of embodiments 1-233, or a pharmaceutically acceptable salt thereof, wherein the compound is of Formula IV-g

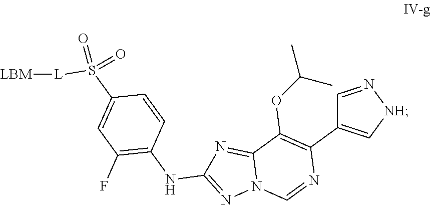

Embodiment 279. The compound of any one of embodiments 1-233, or a pharmaceutically acceptable salt thereof, wherein the compound is of Formula IV-h

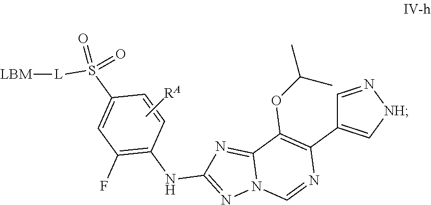

Embodiment 280. The compound of any one of embodiments 1-233, or a pharmaceutically acceptable salt thereof, wherein the compound is of Formula IV-i:

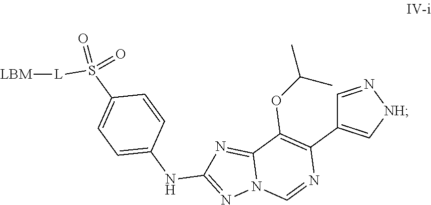

Embodiment 281. The compound of any one of embodiments 1-280, or a pharmaceutically acceptable salt thereof, wherein L is —Z¹—Z²-Z³—Z⁴—Z⁵—wherein:
Z¹ is a bond, C₁₋₆ alkylene, —C(O)NR—, —NR′(CO)—, —S(O)₂NR—, —NR'S(O)₂—, —(O—C₁₋₆ alkylene)ₐ—, —(C₁₋₆ alkylene-O)ₐ—, phenylene, 5-6 membered monocyclic heteroarylene having 1-4 heteroatoms independently selected from nitrogen, oxygen, and sulfur, or 4-11 membered heterocyclylene having 1-2 heteroatoms independently selected from nitrogen, oxygen, and sulfur, where each ring is substituted with 0, 1 or 2 substituents independently selected from C₁₋₆alkyl, C₁₋₆ alkoxy, halo, C₁₋₆ haloalkyl, and C₁₋₆ haloalkoxy;
Z² is a bond, C₁₋₆ alkylene, C₂₋₆ alkynylene, —C(O)—, —C(O)N(R^{cc})—, —NR′(CO)—, —(O—C₁₋₆ alkylene)_{b}-, —(C₁₋₈ alkylene-O)_{b}—, —(O—C₁₋₈ alkylene)_{b}, —C₃₋₁₁ cycloalkylene, 4-11 membered heterocyclylene having 1-2 heteroatoms independently selected from nitrogen, oxygen, and sulfur, where each ring is substituted with 0, 1 or 2 substituents independently selected from C₁₋₆ alkyl, C₁₋₆alkoxy, halo, C₁₋₆haloalkyl, and C₁₋₆haloalkoxy;
Z³ is a bond, C₁₋₆ alkylene, C₂₋₆ alkynylene, —C(O)NR—, —NR′(CO)—, —O—, —NR″—, —(O—C₁₋₈ alkylene)_{c}-, —(C₁₋₈ alkylene-O)_{c}—, C₃-11 cycloalkylene, C₅₋₁₁ spiro cycloalkylene, phenylene, 5-6 membered monocyclic heteroarylene having 1-4 heteroatoms independently selected from nitrogen, oxygen, and sulfur, 4-7 membered monocyclic heterocyclylene having 1-2 heteroatoms independently selected from nitrogen, oxygen, and sulfur, a 5-11 membered spiro heterocyclylene having 1-2 heteroatoms independently selected from nitrogen, oxygen, and sulfur, an 4-10 membered bicyclic heterocyclylene having 1-2 heteroatoms independently selected from nitrogen, oxygen, and sulfur, a 5-10 membered bridged bicyclic saturated or partially unsaturated heterocyclylene having 1-2 heteroatoms independently selected from nitrogen, oxygen, and sulfur, where each ring is substituted with 0, 1 or 2 substituents independently selected from alkyl, alkoxy, halo, haloalkyl, and haloalkoxy;
Z⁴ is a bond, alkylene, alkynylene, -(alkylene-NR″)—, —O—, —C(O)—, —NR″—, —(O-alkylene)ₐ—, -(alkylene-O)ₐ—, C₃₋₁₁ cycloalkylene, C₅₋₁₁ spiro cycloalkylene, phenylene, a 4-7 membered monocyclic heterocyclylene having 1-2 heteroatoms independently selected from nitrogen, oxygen, and sulfur, a 5-11 membered spiro heterocyclylene having 1-2 heteroatoms independently selected from nitrogen, oxygen, and sulfur, an 4-10 membered bicyclic heterocyclylene having 1-2 heteroatoms independently selected from nitrogen, oxygen, and sulfur, a 5-10 membered bridged bicyclic saturated or partially unsaturated heterocyclylene having 1-2 heteroatoms independently selected from nitrogen, oxygen, and sulfur, a 5-6 membered heteroarylene having 1-4 heteroatoms independently selected from nitrogen, oxygen, and sulfur, or an 8-10 membered bicyclic heteroarylene having 1-5 heteroatoms independently selected from nitrogen, oxygen, or sulfur, where each ring is substituted with 0, 1 or 2 substituents independently selected from alkyl, alkoxy, halo, haloalkyl, and haloalkoxy;
Z⁵ is a bond, -alkylene, —NR″—, —O—, —C(O)—, —S(O)₂—, —NR′(CO)—, —C(O)NR—, phenylene, a 4-7 membered monocyclic heterocyclylene having 1-2 heteroatoms independently selected from nitrogen, oxygen, and sulfur, a 5-11 membered spiro heterocyclylene having 1-2 heteroatoms independently selected from nitrogen, oxygen, and sulfur, an 4-10 membered bicyclic heterocyclylene having 1-2 heteroatoms independently selected from nitrogen, oxygen, and sulfur, a 5-10 membered bridged bicyclic saturated or partially unsaturated heterocyclylene having 1-2 heteroatoms independently selected from nitrogen, oxygen, and sulfur, a 5-6 membered heteroarylene having 1-4 heteroatoms independently selected from nitrogen, oxygen, and sulfur, or an 8-10 membered bicyclic heteroarylene having 1-5 heteroatoms independently selected from nitrogen, oxygen, or sulfur, where each ring is substituted with 0, 1 or 2 substituents independently selected from alkyl, alkoxy, halo, haloalkyl, and haloalkoxy;
each R, R′ and R″ is independently selected from H and C₁₋₆ alkyl;
each a, b, c, and d is independently an integer selected from 1, 2, 3, 4, 5 and 6;
and each alkylene is substituted with 0, 1, 2, 3 or 4 substituents where 0, 1, 2, or 3 substituents are independently selected from fluoro and deuterium, and 0 or 1 substituents are carboxy;
provided that at least one of —Z¹—Z²-Z³—Z⁴—Z⁵—is not a bond.

Embodiment 282. The compound of any one of embodiments 1-280 or a pharmaceutically acceptable salt thereof, wherein L is selected from the group consisting of

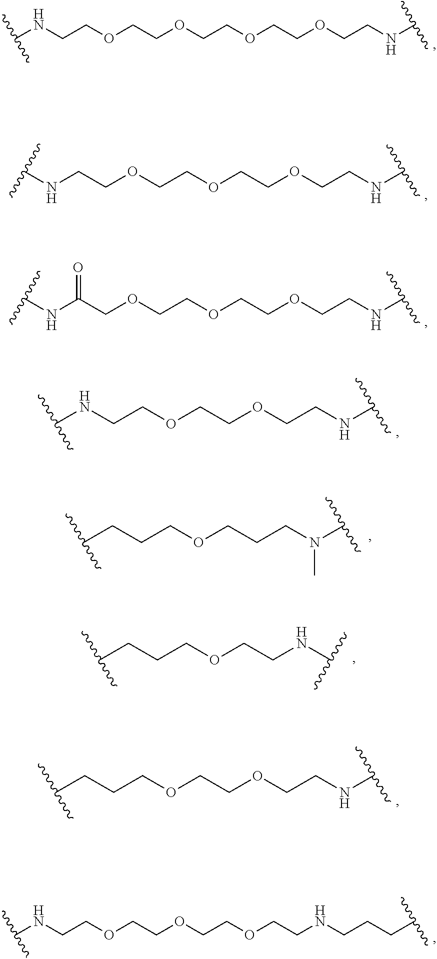

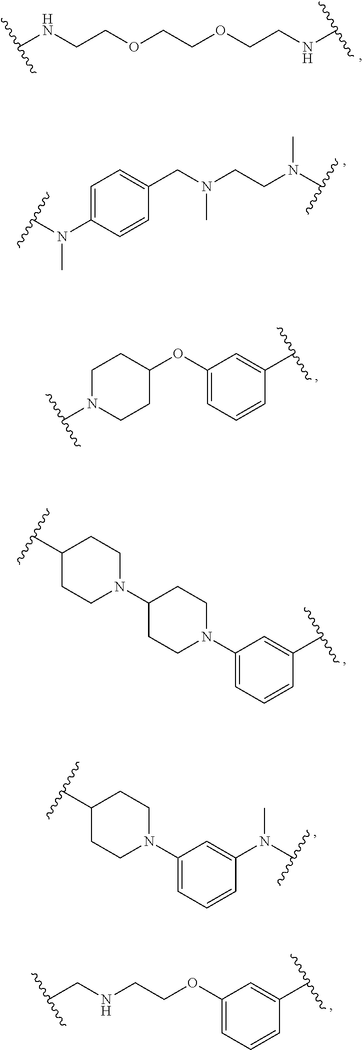 [SMILES: *NCCOCCOCCN* ; *N(C)CCN(C)Cc1ccc(N(*)C)cc1 ; *c1cccc(OC2CCN(*)CC2)c1 ; *c1cccc(N2CCC(N3CCC(*)CC3)CC2)c1 ; *C1CCN(c2cccc(N(*)C)c2)CC1 ; *CNCCOc1cccc(*)c1]

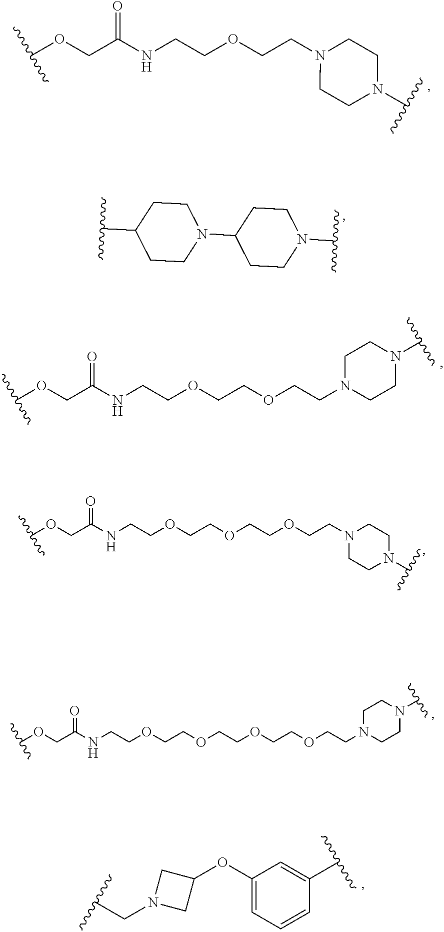 [SMILES: *OCC(=O)NCCOCCN1CCN(*)CC1 ; *C1CCN(C2CCN(*)CC2)CC1 ; *OCC(=O)NCCOCCOCCN1CCN(*)CC1 ; *OCC(=O)NCCOCCOCCOCCN1CCN(*)CC1 ; *OCC(=O)NCCOCCOCCOCCOCCN1CCN(*)CC1 ; *CN1CC(Oc2cccc(*)c2)C1]

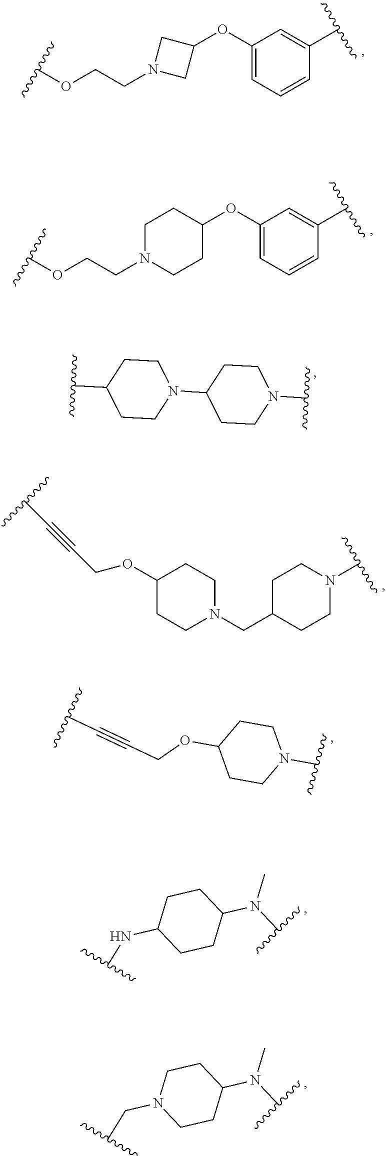

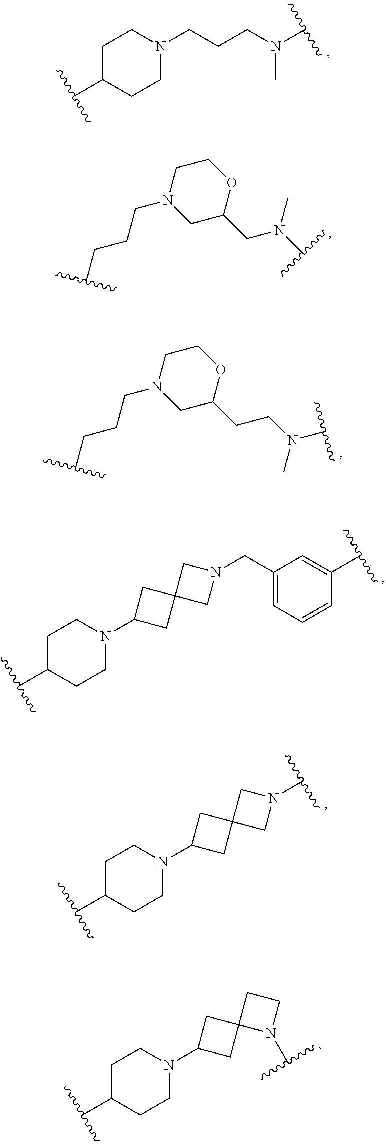 [SMILES: *C1CCN(CCCN(*)C)CC1 ; *CCCN1CCOC(CN(*)C)C1 ; *CCCN1CCOC(CCN(*)C)C1 ; *c1cccc(CN2CC3(CC(N4CCC(*)CC4)C3)C2)c1 ; *C1CCN(C2CC3(C2)CN(*)C3)CC1 ; *C1CCN(C2CC3(CCN3*)C2)CC1]

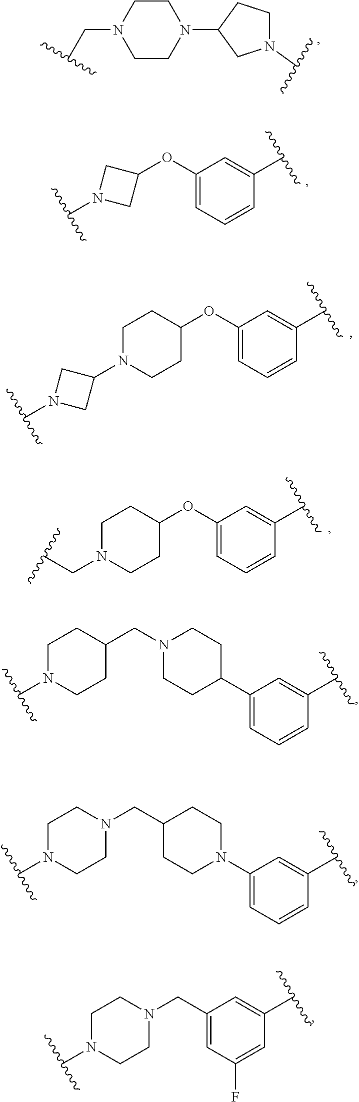

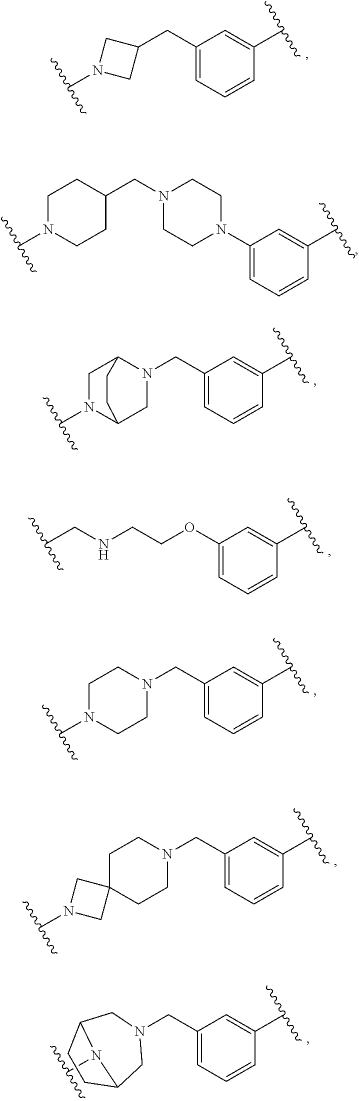

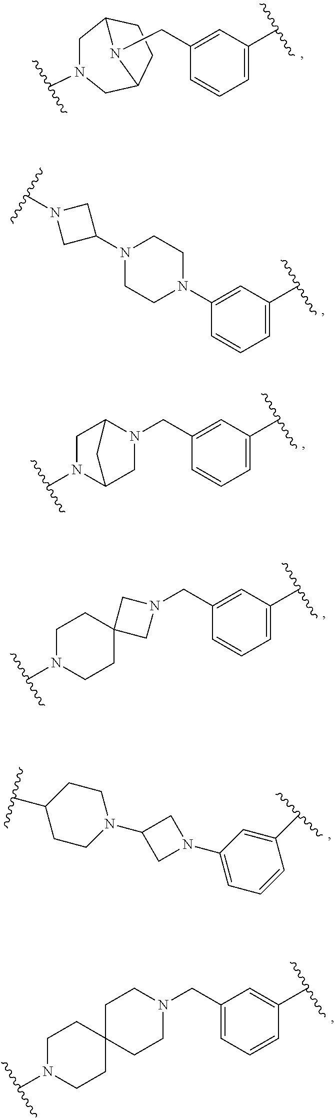 [SMILES: *c1cccc(CN2C3CCC2CN(*)C3)c1 ; *c1cccc(N2CCN(C3CN(*)C3)CC2)c1 ; *c1cccc(CN2CC3CC2CN3*)c1 ; *c1cccc(CN2CC3(CCN(*)CC3)C2)c1 ; *c1cccc(N2CC(N3CCC(*)CC3)C2)c1 ; *c1cccc(CN2CCC3(CCN(*)CC3)CC2)c1]

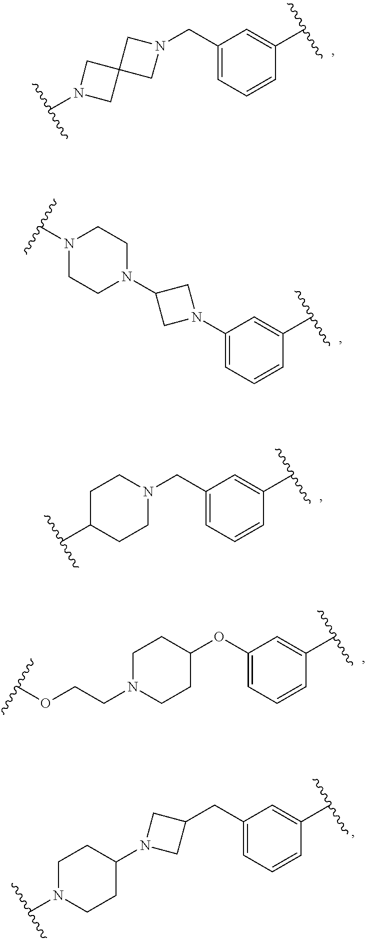 [SMILES: *c1cccc(CN2CC3(CN(*)C3)C2)c1 ; *c1cccc(N2CC(N3CCN(*)CC3)C2)c1 ; *c1cccc(CN2CCC(*)CC2)c1 ; *OCCN1CCC(Oc2cccc(*)c2)CC1 ; *c1cccc(CC2CN(C3CCN(*)CC3)C2)c1]

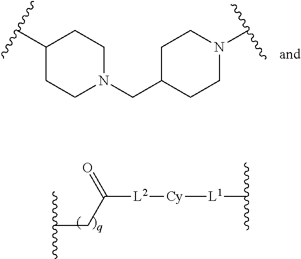

wherein the left attachment point connects to LBM and the right attachment point connects to —X— of Formula A-I or —S(O)₂— of Formula I; and wherein
L¹ and L² are each independently selected from a bond and —N(R′), wherein R′ is selected from H and C₁₋₆ alkyl; and
q is 0, 1, 2, 3, 4, 5, 6, 7, 8, 9 or 10.

Embodiment 283. The compound of any one of embodiments 1-280, or a pharmaceutically acceptable salt thereof, wherein L is selected from

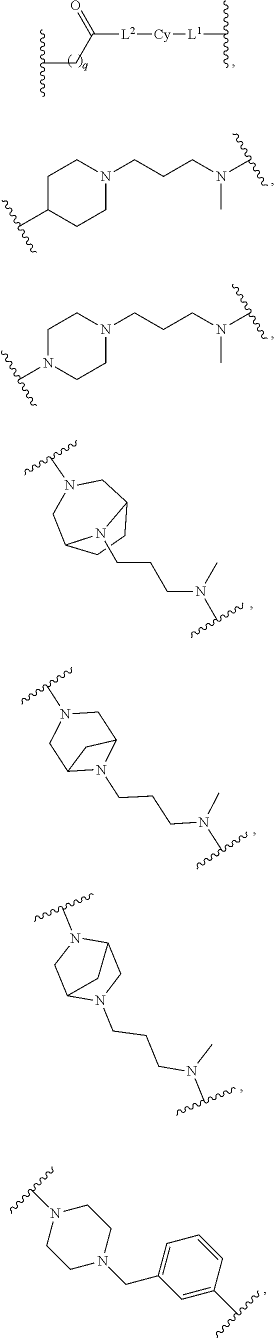

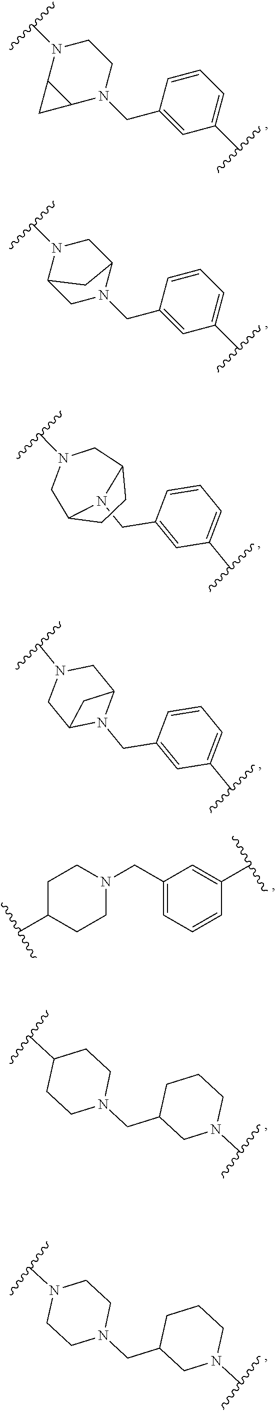

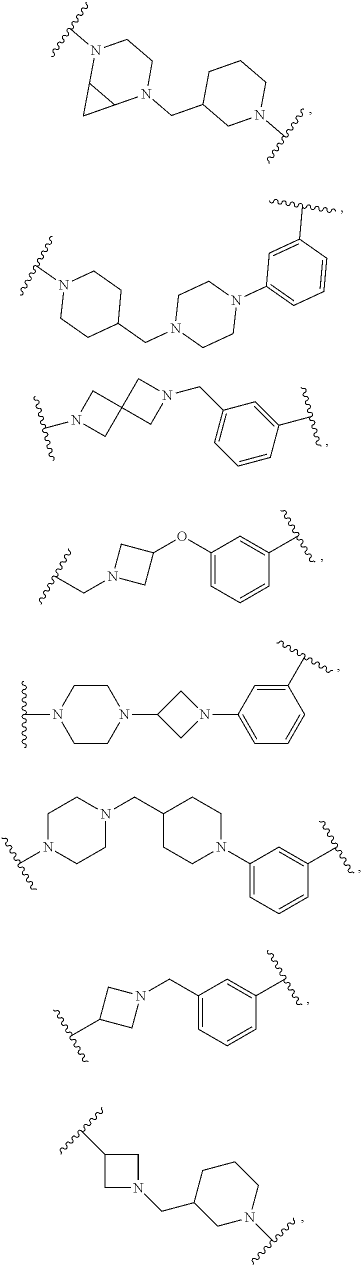

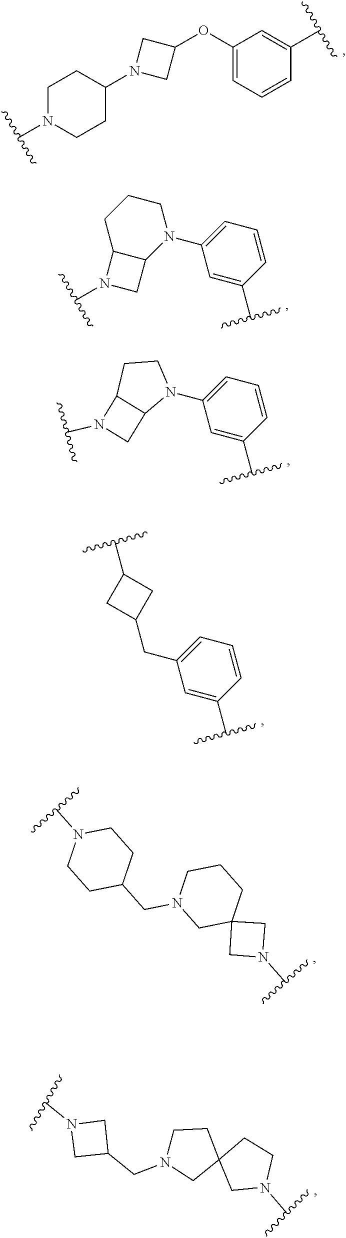 [SMILES: *c1cccc(OC2CN(C3CCN(*)CC3)C2)c1 ; *c1cccc(N2CCCC3C2CN3*)c1 ; *c1cccc(N2CCC3C2CN3*)c1 ; *c1cccc(CC2CC(*)C2)c1 ; *N1CCC(CN2CCCC3(CN(*)C3)C2)CC1 ; *N1CC(CN2CCC3(CCN(*)C3)C2)C1]

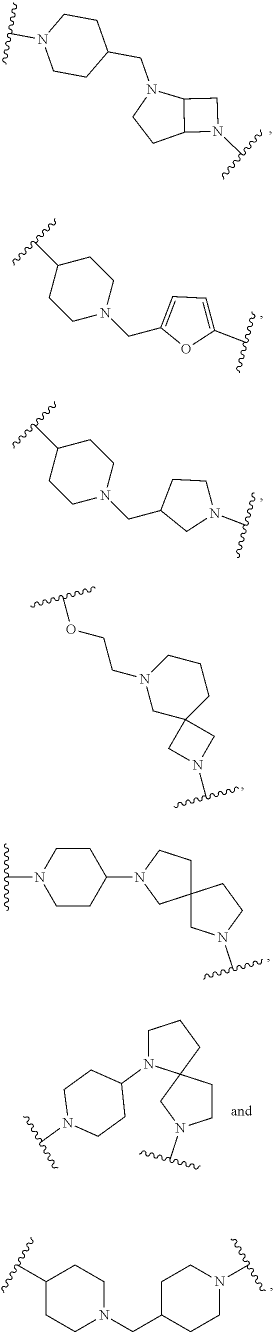

each substituted with 0, 1, 2 or 3 instances of R⁷, wherein each R⁷ is independently selected from —C₁₋₄ alkyl and halo; wherein the left attachment point connects to LBM and the right attachment point connects to the —X— group of Formula A-I or —S(O)₂— group of Formula I and wherein
L¹ and L² are each independently selected from a bond and —N(R′), wherein R′ is selected from H and C₁₋₆ alkyl; and
q is 0, 1,2,3,4,5,6,7,8,9 or 10.

Embodiment 284. The compound of any one of embodiments 1-280, or a pharmaceutically acceptable salt thereof, wherein L is selected from:

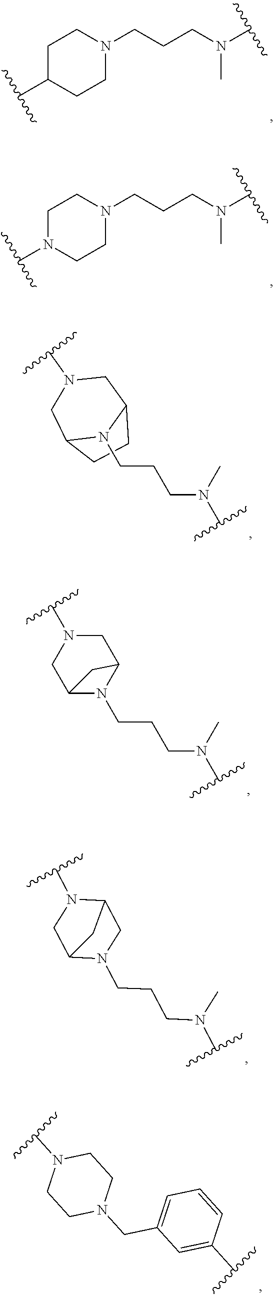 [SMILES: *C1CCN(CCCN(*)C)CC1 ; *N(C)CCCN1CCN(*)CC1 ; *N(C)CCCN1C2CCC1CN(*)C2 ; *N(C)CCCN1C2CC1CN(*)C2 ; *N(C)CCCN1CC2CC1CN2* ; *c1cccc(CN2CCN(*)CC2)c1]

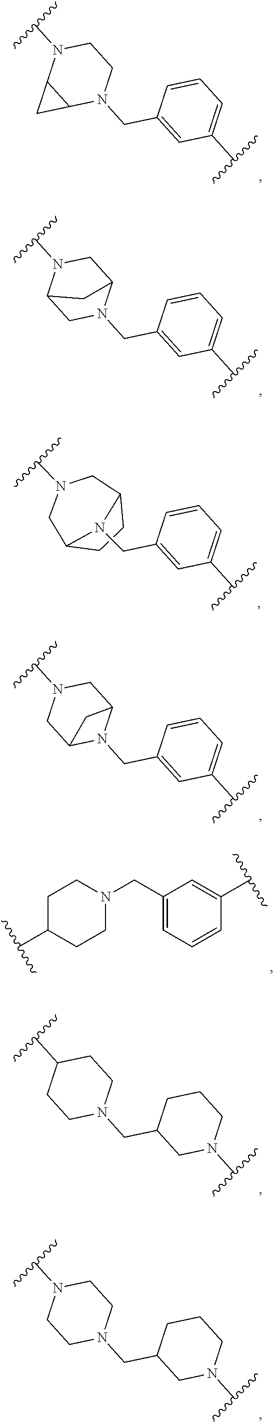

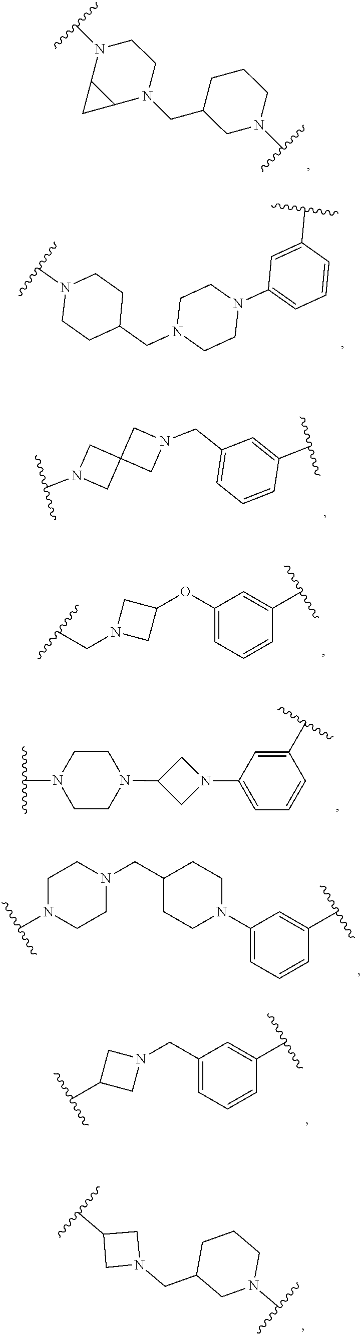

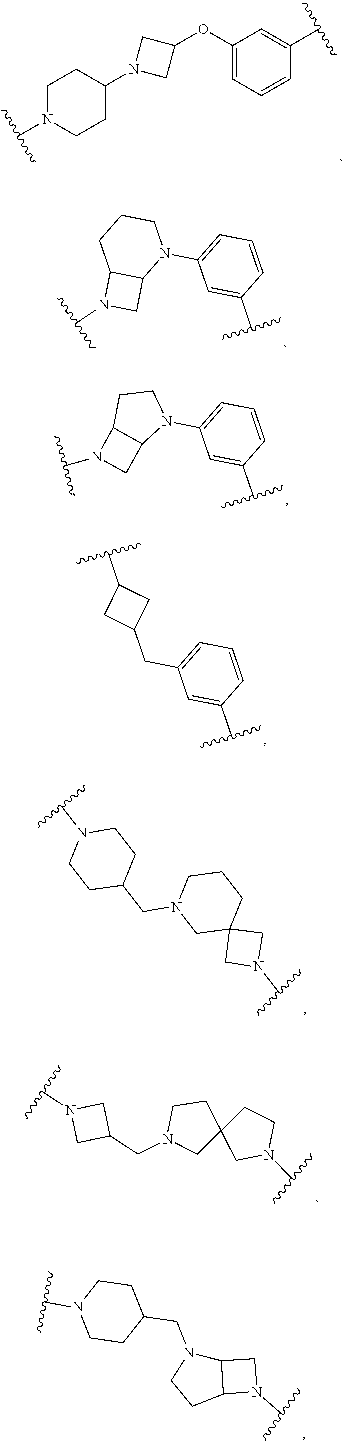

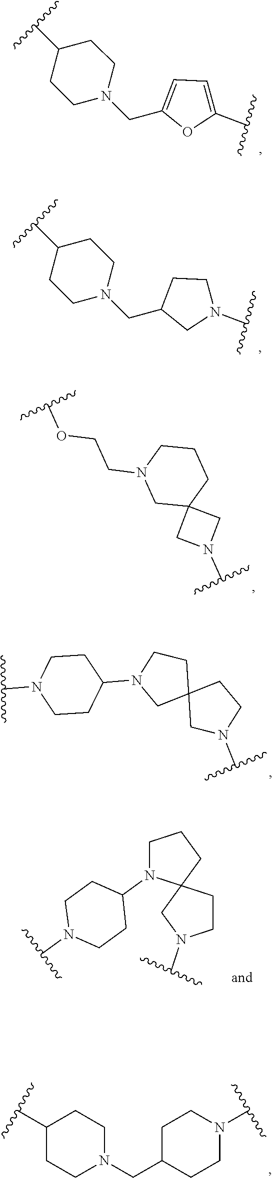 [SMILES: *c1ccc(CN2CCC(*)CC2)o1 ; *C1CCN(CC2CCN(*)C2)CC1 ; *OCCN1CCCC2(CN(*)C2)C1 ; *N1CCC(N2CCC3(CCN(*)C3)C2)CC1 ; *N1CCC(N2CCCC23CCN(*)C3)CC1 ; *C1CCN(CC2CCN(*)CC2)CC1]

each substituted with 0, 1, 2 or 3 instances of R⁷, wherein each R⁷ is independently selected from —C₁₋₄ alkyl and halo;
wherein the left attachment point connects to LBM and the right attachment point connects to the —X— group of Formula A-I or —S(O)₂— group of Formula I.

Embodiment 285. The compound of embodiment 284, or a pharmaceutically acceptable salt thereof, wherein L is selected from:

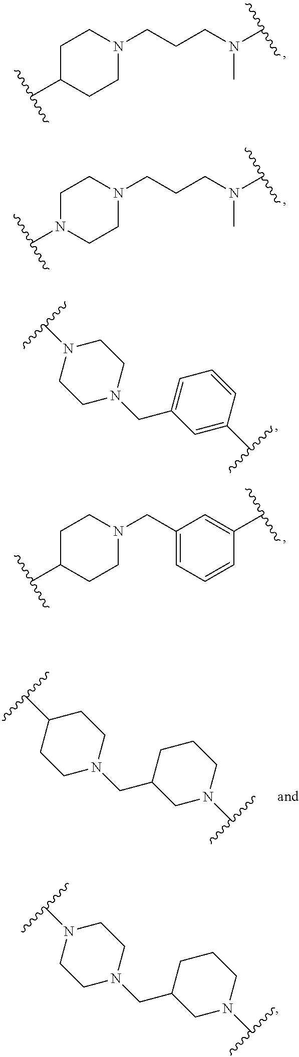 [SMILES: *C1CCN(CCCN(*)C)CC1 ; *N(C)CCCN1CCN(*)CC1 ; *c1cccc(CN2CCN(*)CC2)c1 ; *c1cccc(CN2CCC(*)CC2)c1 ; *C1CCN(CC2CCCN(*)C2)CC1 ; *N1CCN(CC2CCCN(*)C2)CC1]

each substituted with 0, 1, 2 or 3 instances of R⁷, wherein each R⁷ is independently selected from —C₁₋₄ alkyl and halo;
wherein the left attachment point connects to LBM and the right attachment point connects to the —X— group of Formula A-I or —S(O)₂— group of Formula I.

Embodiment 286. The compound of any one of embodiments 1-280, or a pharmaceutically acceptable salt thereof, wherein L is selected from

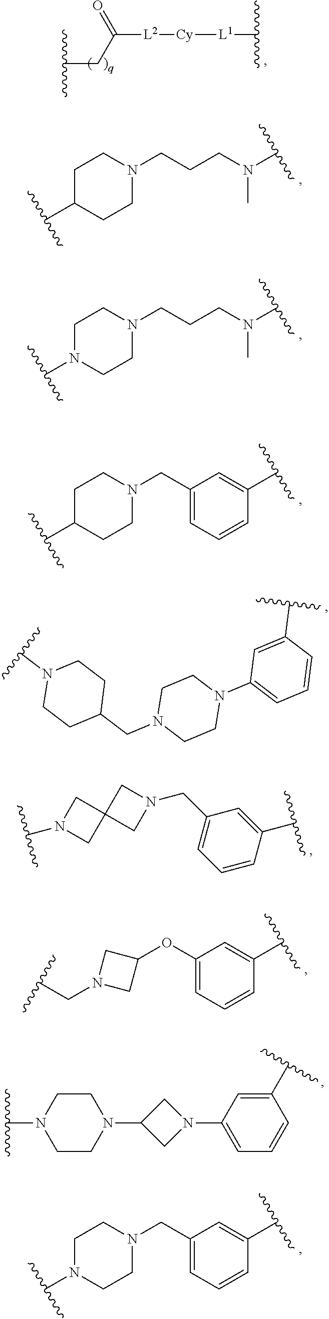

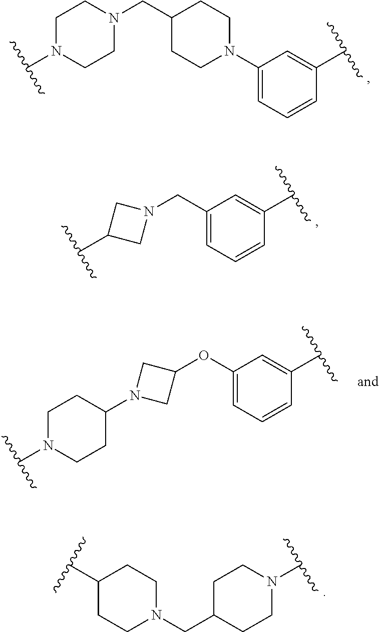 [SMILES: *c1cccc(N2CCC(CN3CCN(*)CC3)CC2)c1 ; *c1cccc(CN2CC(*)C2)c1 ; *c1cccc(OC2CN(C3CCN(*)CC3)C2)c1 ; *C1CCN(CC2CCN(*)CC2)CC1]

wherein the left attachment point connects to LBM and the right attachment point connects to the —X—group of Formula A-I or —S(O)₂— group of Formula I and wherein
L¹ and L² are each independently selected from a bond and —N(R′), wherein R′ is selected from H and C₁₋₆ alkyl; and
q is 0, 1, 2, 3, 4, 5, 6, 7, 8, 9 or 10.

Embodiment 287. The compound of any one of embodiments 1-280, or a pharmaceutically acceptable salt thereof wherein L is selected from

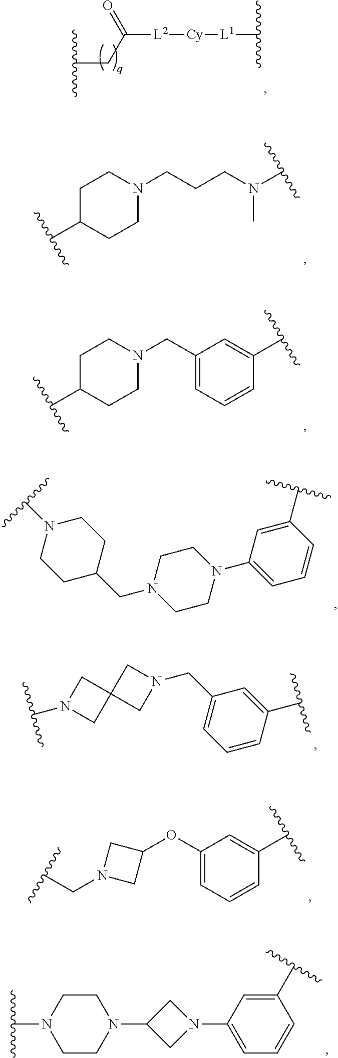

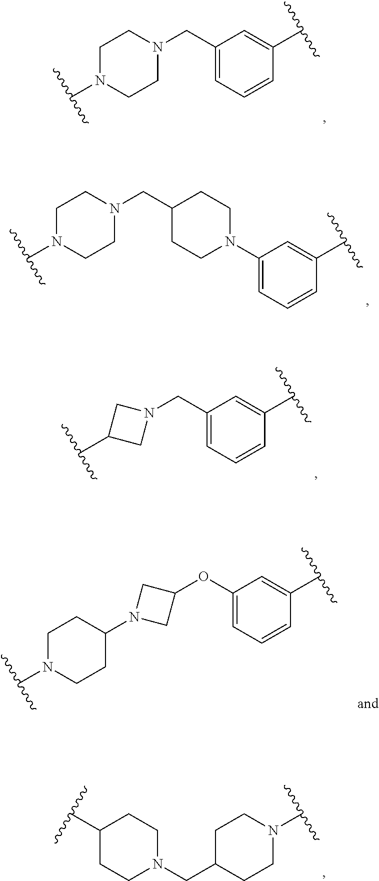 [SMILES: *c1cccc(CN2CCN(*)CC2)c1 ; *c1cccc(N2CCC(CN3CCN(*)CC3)CC2)c1 ; *c1cccc(CN2CC(*)C2)c1 ; *c1cccc(OC2CN(C3CCN(*)CC3)C2)c1 ; *C1CCN(CC2CCN(*)CC2)CC1]

wherein the left attachment point connects to LBM and the right attachment point connects to the —X— group of Formula A-I or —S(O)₂— group of Formula I and wherein
L¹ and L² are each independently selected from a bond and —N(R′), wherein R′ is selected from H and C₁₋₆ alkyl; and
q is 0, 1, 2, 3, 4, 5, 6, 7, 8, 9 or 10.

Embodiment 288. The compound of any one of embodiments 1-280, or a pharmaceutically acceptable salt thereof, wherein L is selected from:

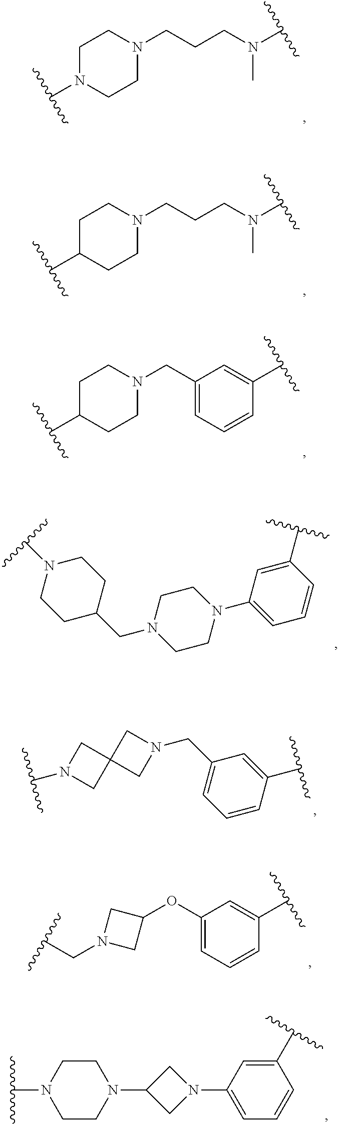

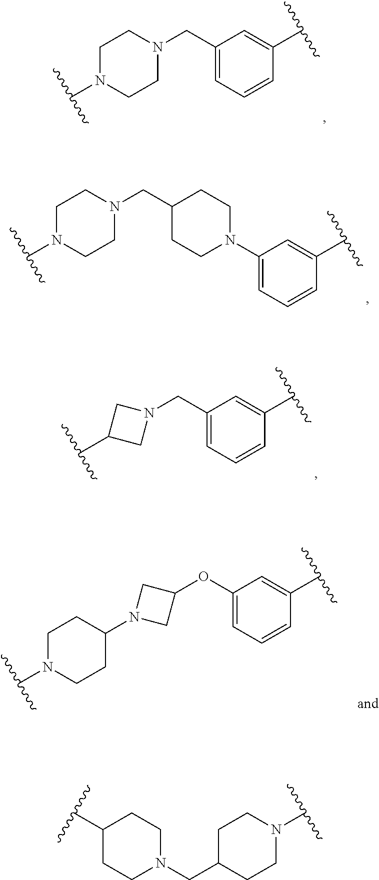 [SMILES: *c1cccc(CN2CCN(*)CC2)c1 ; *c1cccc(N2CCC(CN3CCN(*)CC3)CC2)c1 ; *c1cccc(CN2CC(*)C2)c1 ; *c1cccc(OC2CN(C3CCN(*)CC3)C2)c1 ; *C1CCN(CC2CCN(*)CC2)CC1]

wherein the left attachment point connects to LBM and the right attachment point connects to the —X— group of Formula A-I or —S(O)₂— group of Formula I.

Embodiment 289. The compound of any one of embodiments 1-280, or a pharmaceutically acceptable salt thereof, wherein L is selected from:

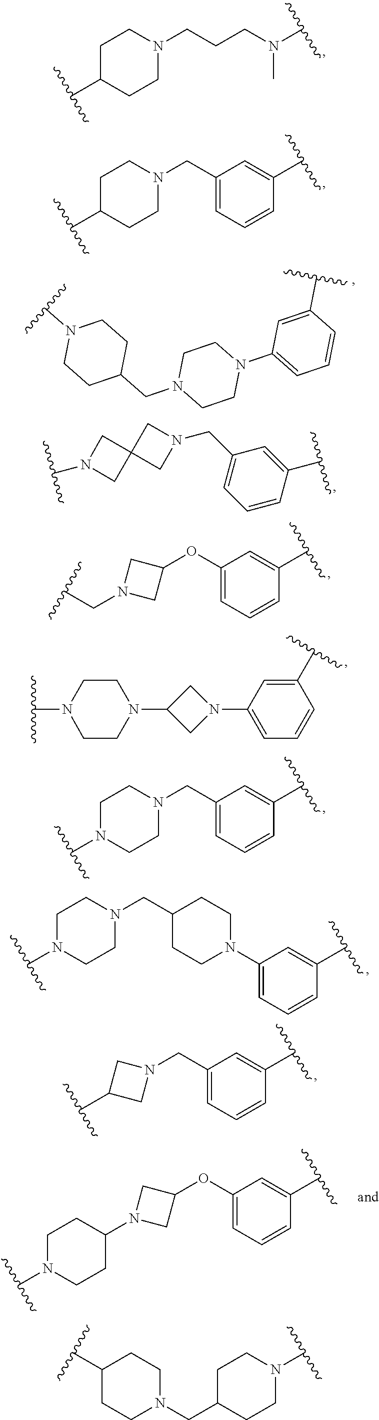

wherein the left attachment point connects to LBM and the right attachment point connects to the —X— group of Formula A-I or —S(O)₂— group of Formula I.

Embodiment 290. The compound of any one of embodiments 1-280, or a pharmaceutically acceptable salt thereof, wherein L is selected from:

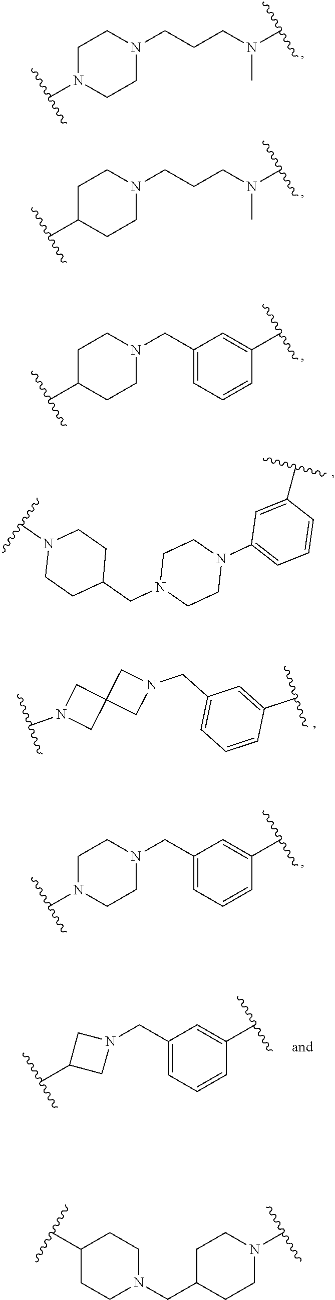

wherein the left attachment point connects to LBM and the right attachment point connects to the —X— group of Formula A-I or —S(O)₂— group of Formula I.

Embodiment 291. The compound of any one of embodiments 1-280, or a pharmaceutically acceptable salt thereof, wherein L is

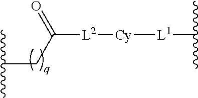

wherein the left attachment point connects to LBM and the right attachment point connects to the —X— group of Formula A-I or —S(O)₂— group of Formula I and wherein
L¹ and L² are each independently selected from a bond and —N(R′), wherein R′ is selected from H and C₁₋₆ alkyl; and
q is 0, 1,2,3,4,5,6,7,8,9 or 10.

Embodiment 292. The compound of any one of embodiments 1-280, or a pharmaceutically acceptable salt thereof, wherein L is selected from

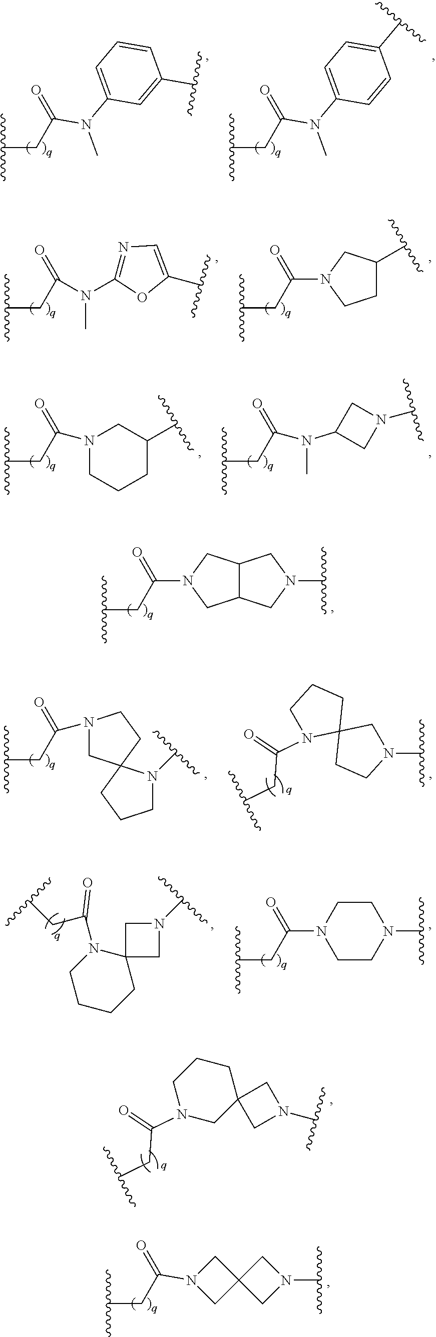

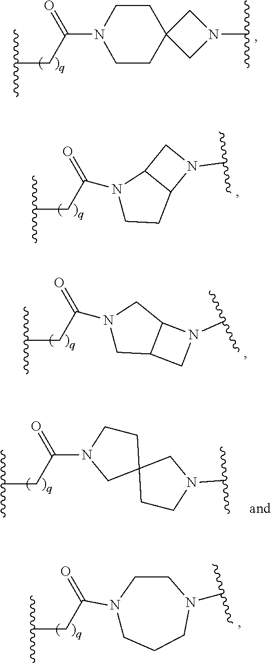

wherein the left attachment point connects to LBM and the right attachment point connects to the —X— group of Formula A-I or —S(O)₂— group of Formula I.

Embodiment 293. The compound of any one of embodiments 1-280, or a pharmaceutically acceptable salt thereof, wherein L is selected from:

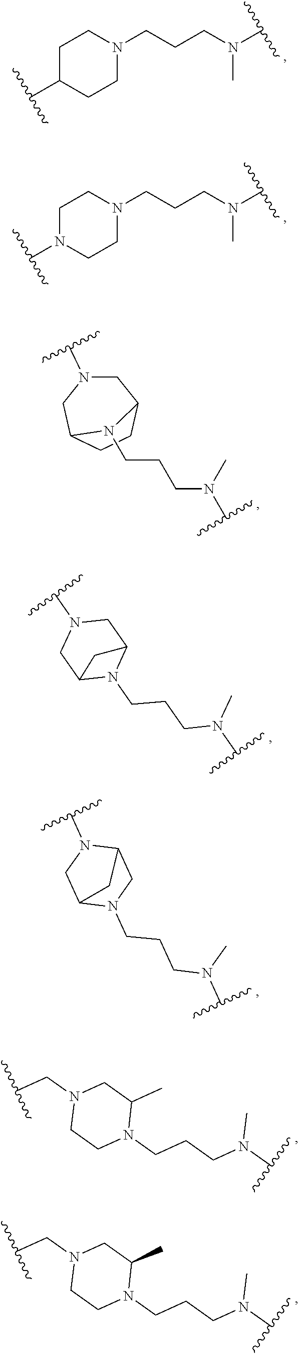

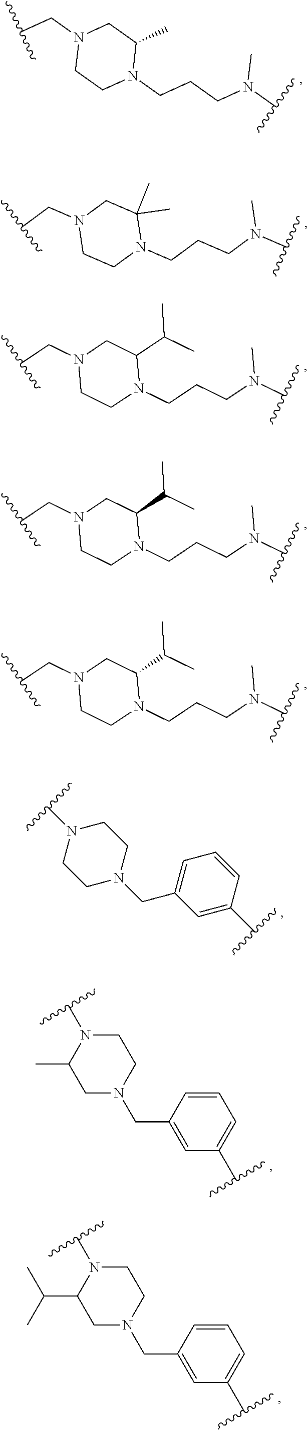

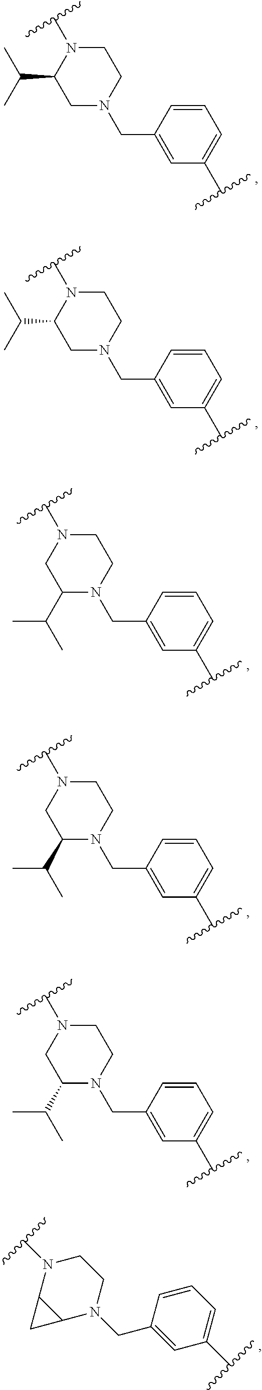 [SMILES: *c1cccc(CN2CCN(*)[C@H](C(C)C)C2)c1 ; *c1cccc(CN2CCN(*)[C@@H](C(C)C)C2)c1 ; *c1cccc(CN2CCN(*)CC2C(C)C)c1 ; *c1cccc(CN2CCN(*)C[C@@H]2C(C)C)c1 ; *c1cccc(CN2CCN(*)C[C@H]2C(C)C)c1 ; *c1cccc(CN2CCN(*)C3CC32)c1]

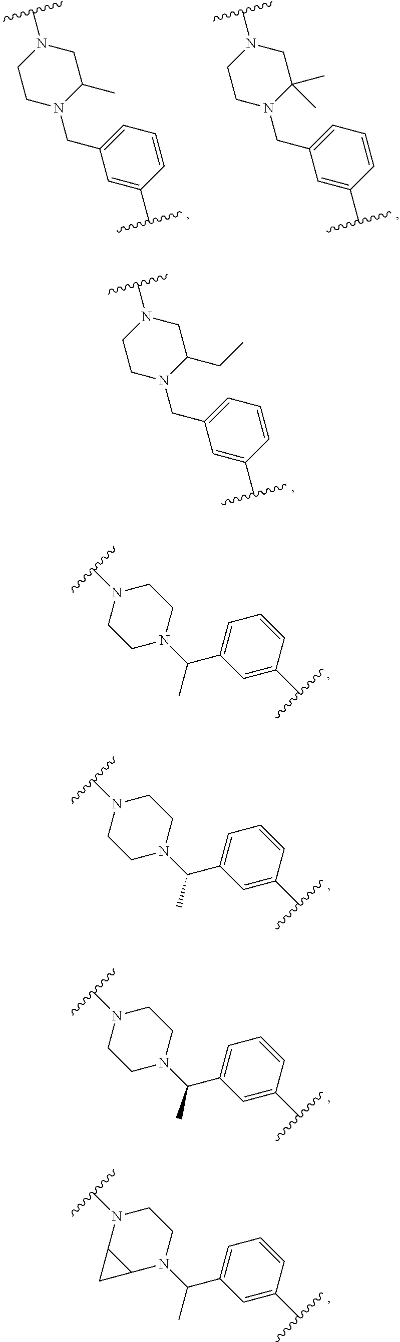

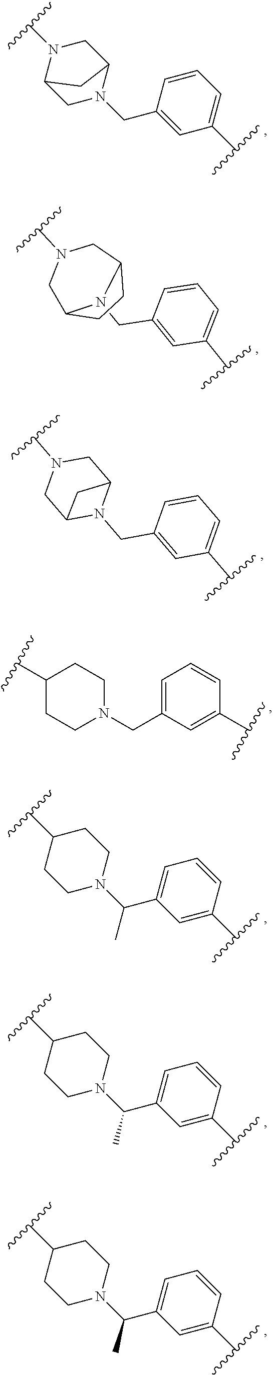

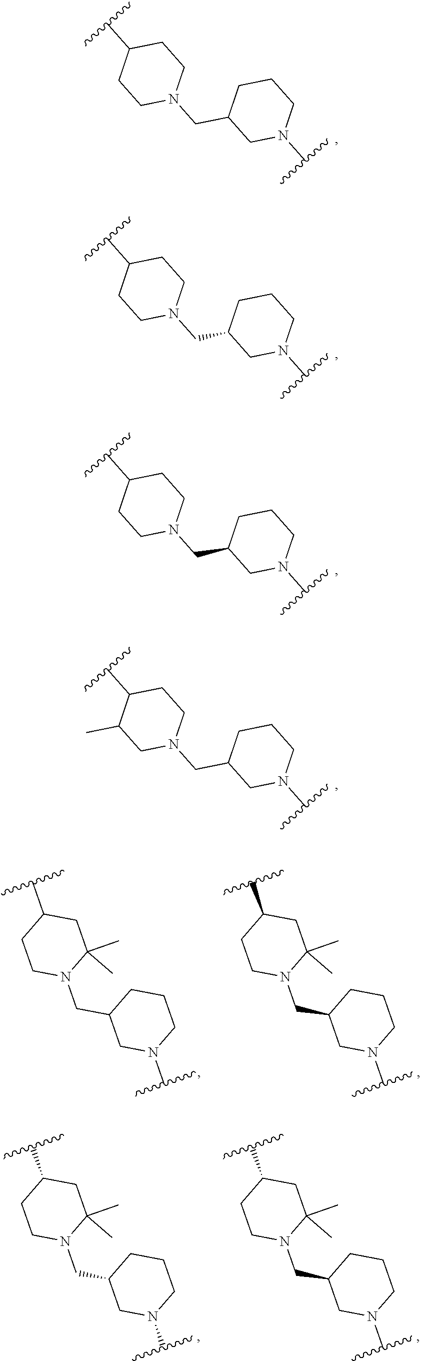

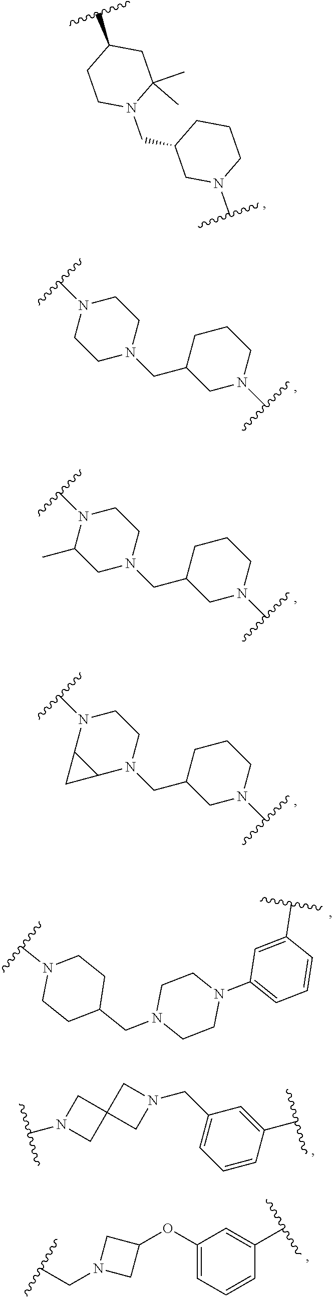

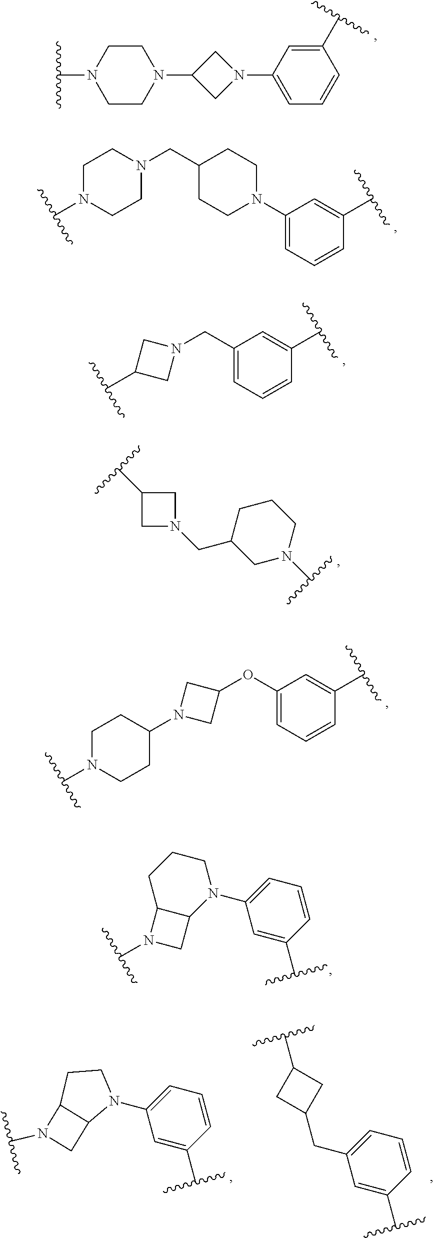

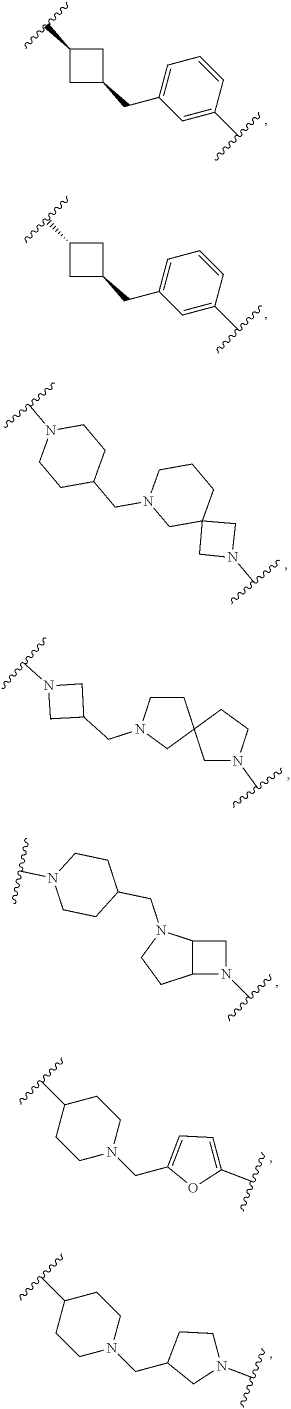

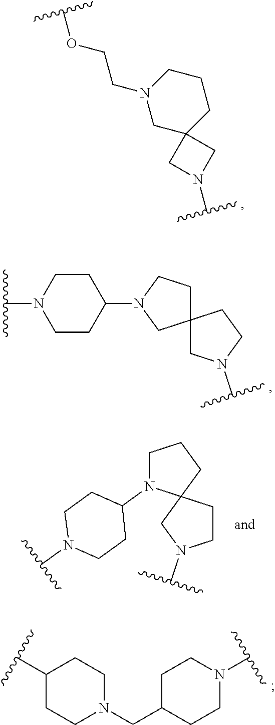 [SMILES: *OCCN1CCCC2(CN(*)C2)C1 ; *N1CCC(N2CCC3(CCN(*)C3)C2)CC1 ; *N1CCC(N2CCCC23CCN(*)C3)CC1 ; *C1CCN(CC2CCN(*)CC2)CC1]

wherein the left attachment point connects to LBM and the right attachment point connects to the —X— group of Formula A-I or —S(O)₂— group of Formula I.

Embodiment 294. The compound of any one of embodiments 1-280, wherein —L—X— is selected from the group consisting of:

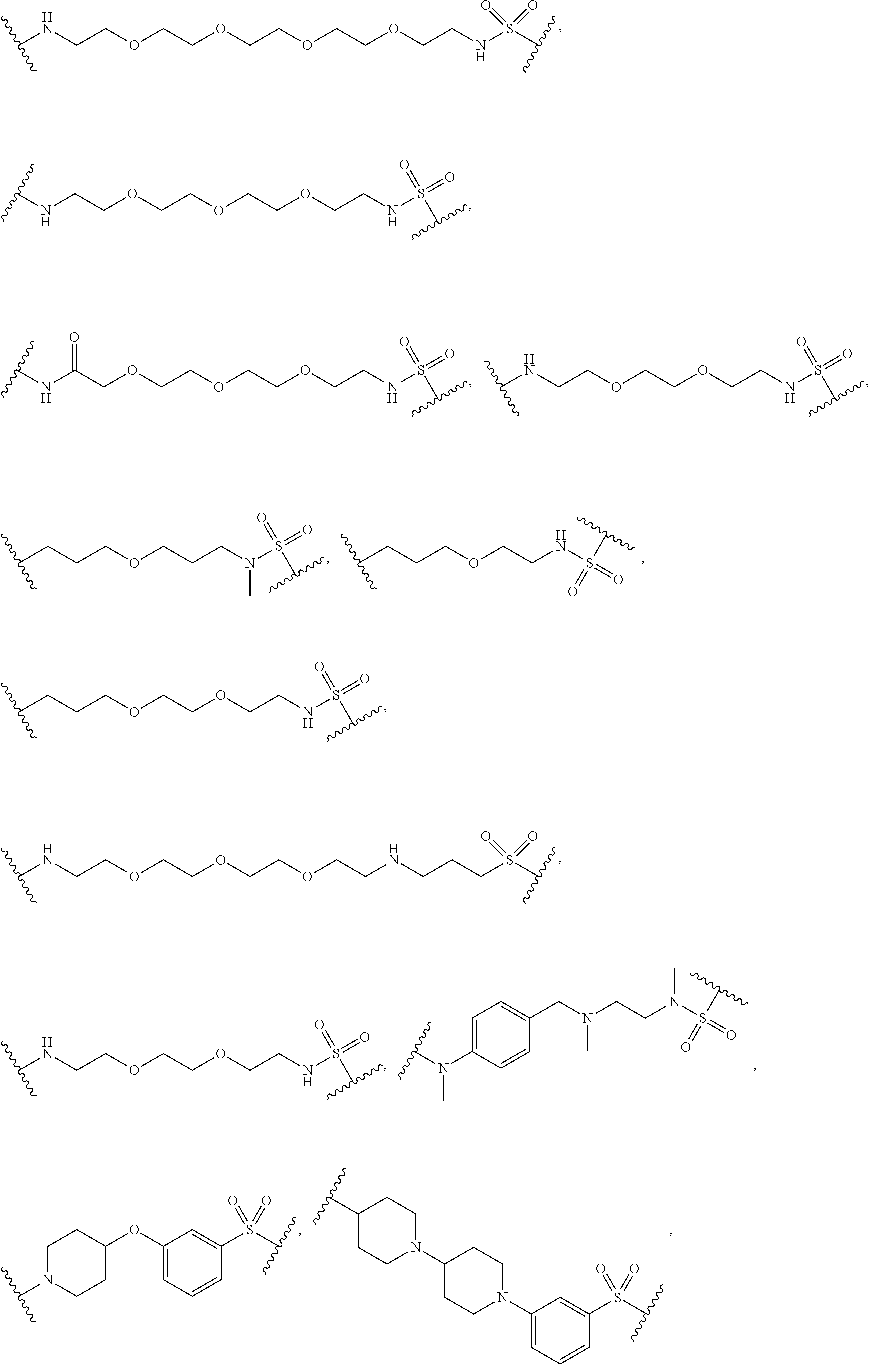

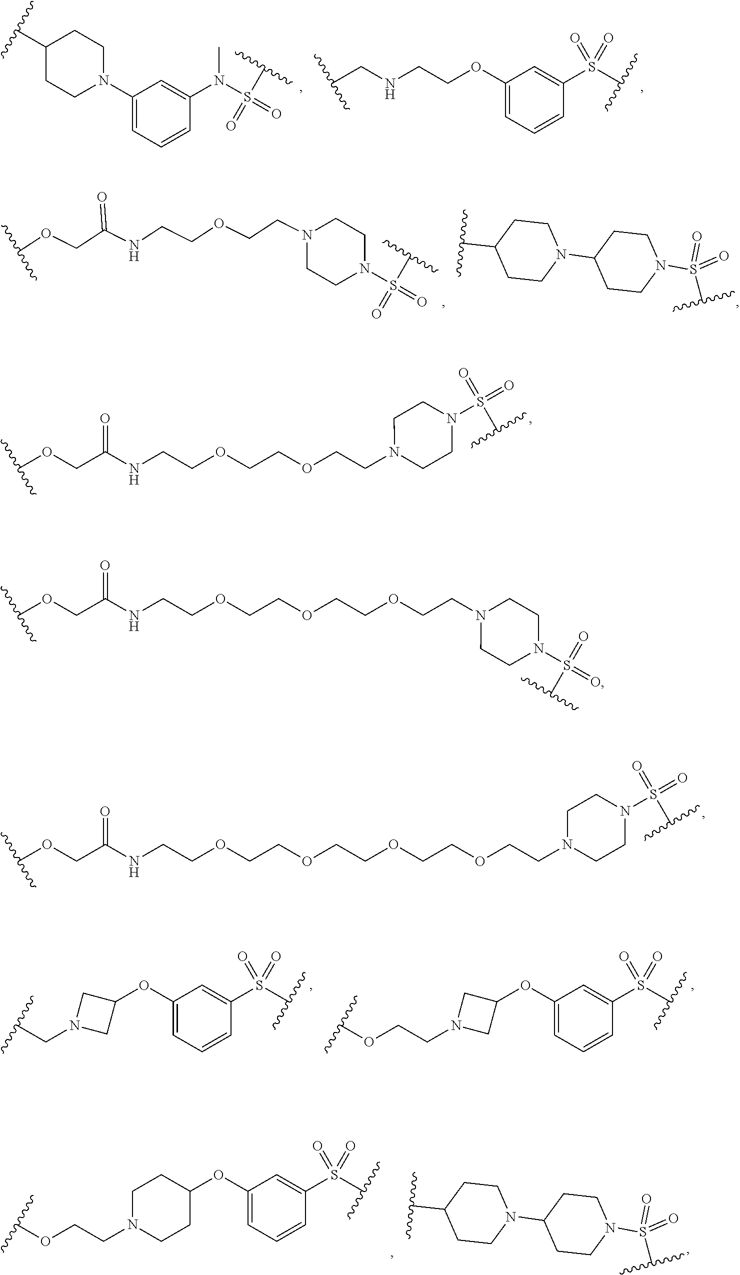

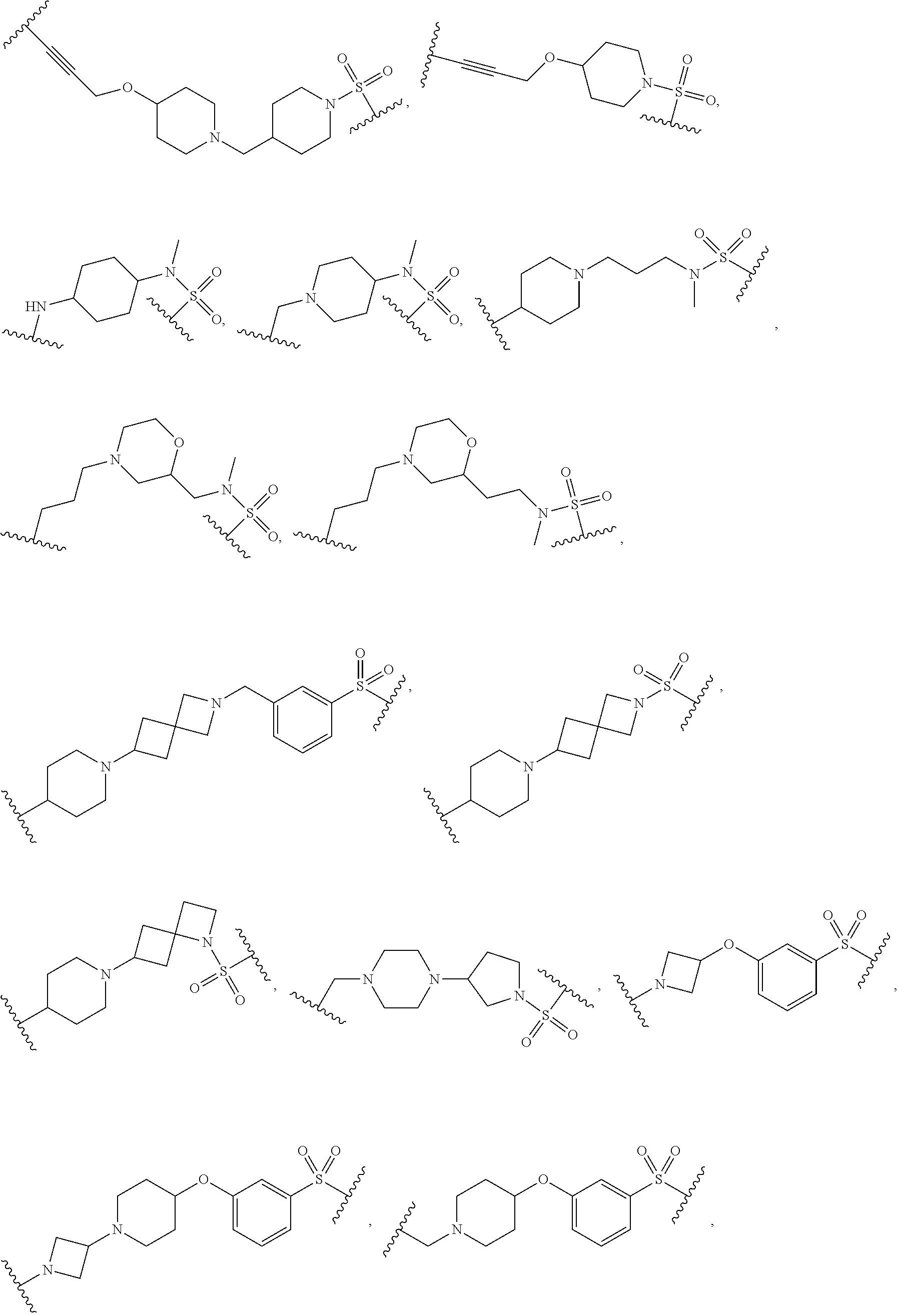

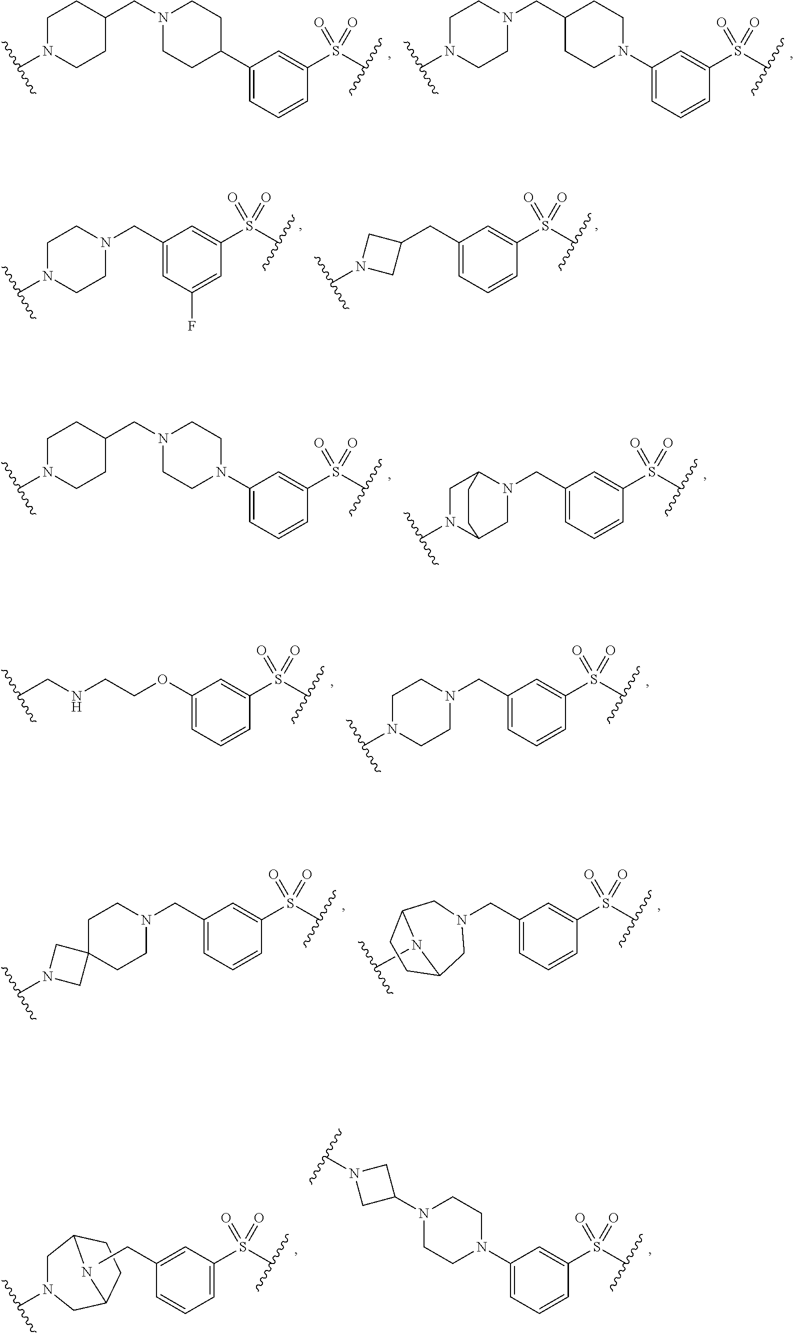

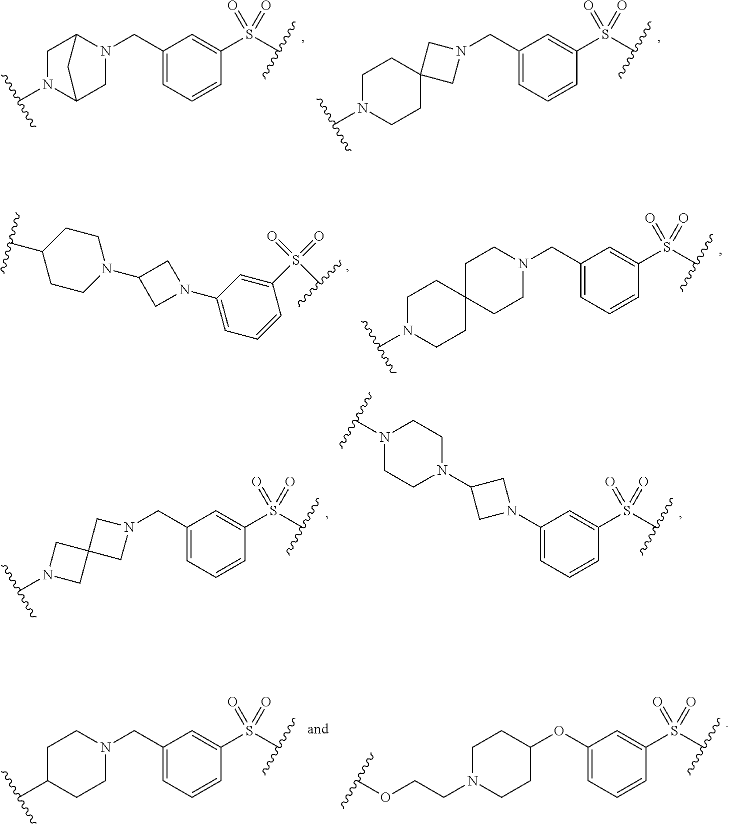

Embodiment 295. The compound of any one of embodiments 1-280, wherein —L—X— is selected from the group consisting of:

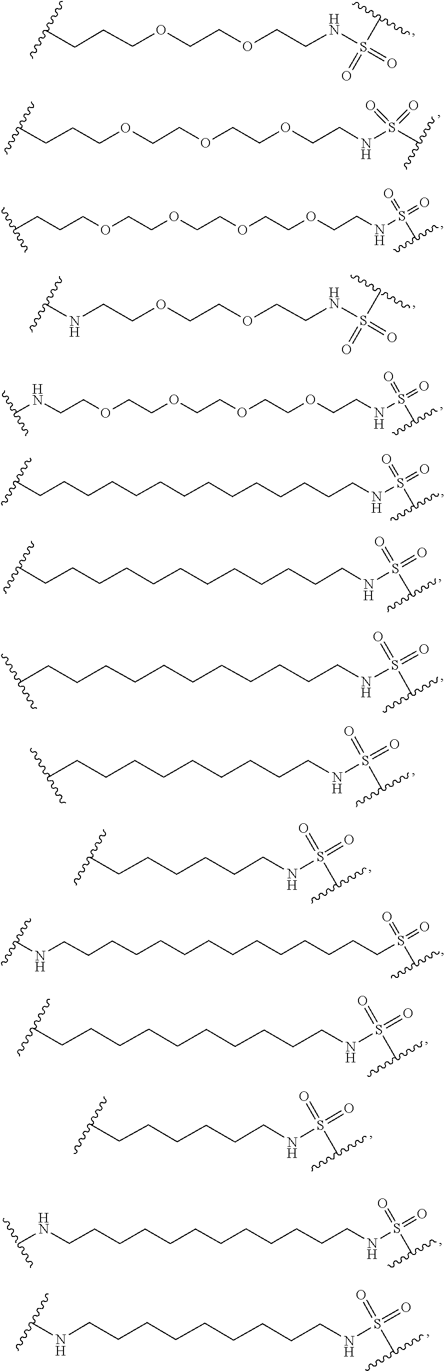

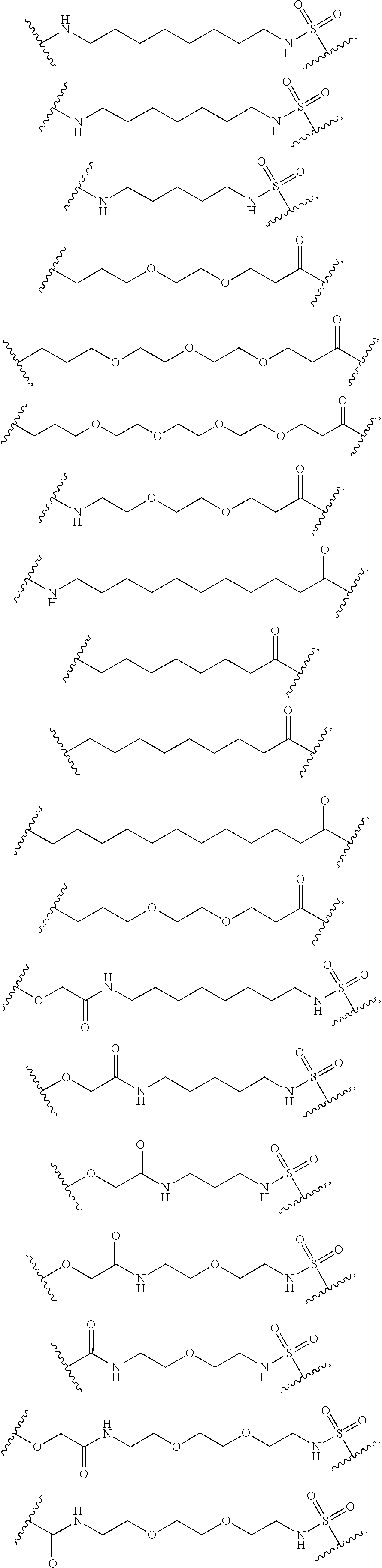

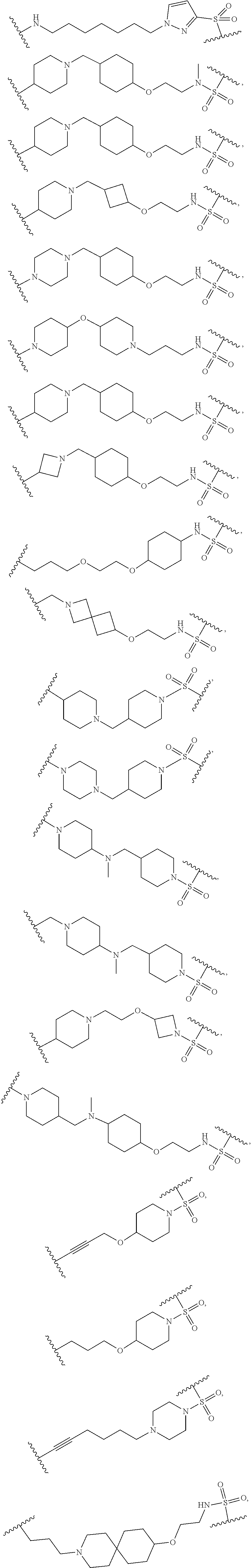

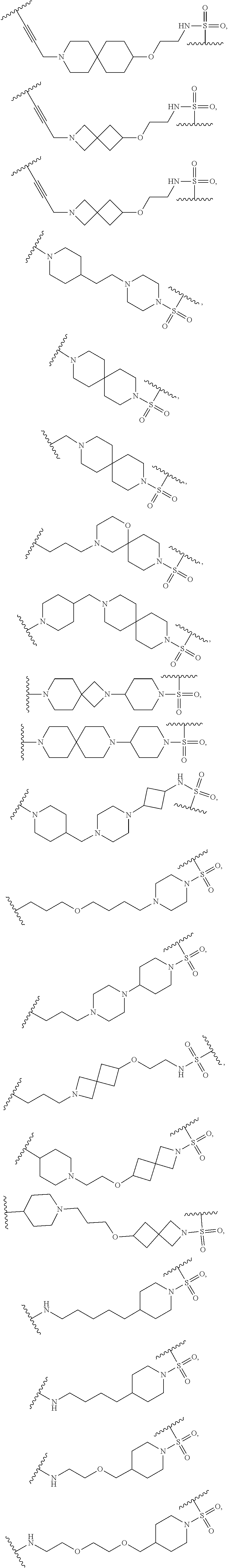

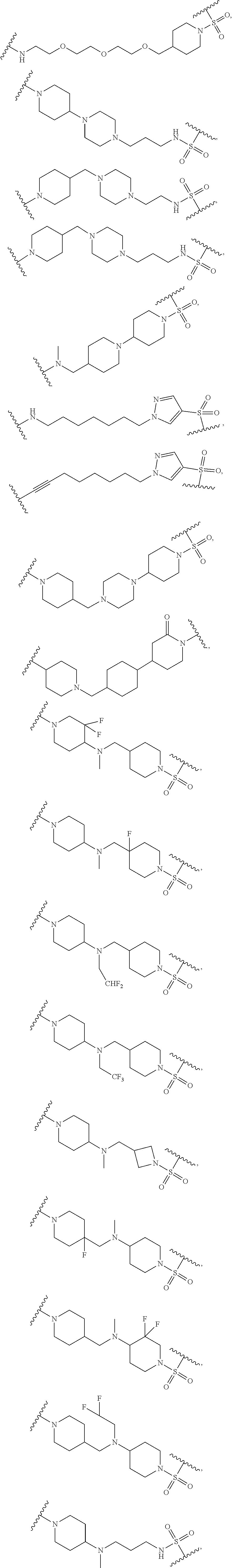

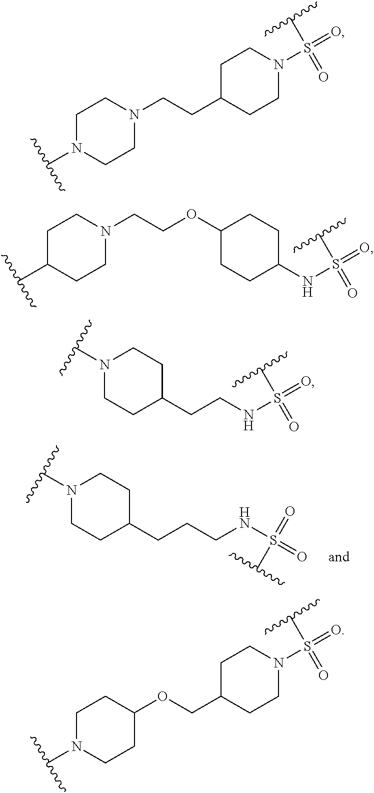 [SMILES: *N1CCN(CCC2CCN(S(*)(=O)=O)CC2)CC1 ; *C1CCN(CCOC2CCC(NS(*)(=O)=O)CC2)CC1 ; *N1CCC(CCNS(*)(=O)=O)CC1 ; *N1CCC(CCCNS(*)(=O)=O)CC1 ; *N1CCC(OCC2CCN(S(*)(=O)=O)CC2)CC1]

Embodiment 296. The compound of any one of embodiments 1-280, or a pharmaceutically acceptable salt thereof, wherein L is selected from List L, wherein either end of L can be attached to X.

Embodiment 297. The compound of any one of embodiments 1-280, or a pharmaceutically acceptable salt thereof, wherein L is

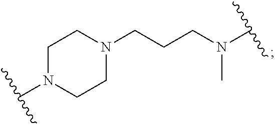 [SMILES: *N(C)CCCN1CCN(*)CC1]

wherein the left attachment point connects to LBM and the right attachment point connects to the —X— group of Formula A-I or —S(O)₂— group of Formula I.

Embodiment 298. The compound of any one of embodiments 1-280, or a pharmaceutically acceptable salt thereof, wherein L is

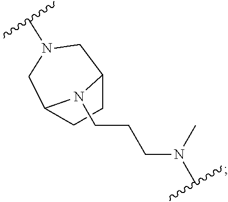 [SMILES: *N(C)CCCN1C2CCC1CN(*)C2]

wherein the left attachment point connects to LBM and the right attachment point connects to the —X— group of Formula A-I or —S(O)₂— group of Formula I.

Embodiment 299. The compound ofany one of embodiments 1-280, or a pharmaceutically acceptable salt thereof, wherein L is

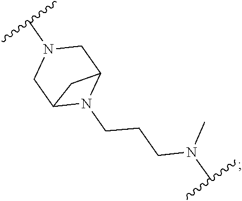 [SMILES: *N(C)CCCN1C2CC1CN(*)C2]

wherein the left attachment point connects to LBM and the night attachment point connects to the —X— group of Formula A-I or —S(O)₂— group of Formula I.

Embodiment 300. The compound of any one of embodiments 1-280, or a pharmaceutically acceptable salt thereof, wherein L is

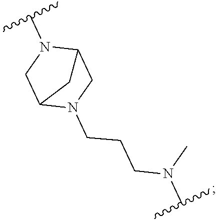 [SMILES: *N(C)CCCN1CC2CC1CN2*]

wherein the left attachment point connects to LBM and the right attachment point connects to the —X— group of Formula A-I or —S(O)₂— group of Formula I.

Embodiment 301. The compound of any one of embodiments 1-280, or a pharmaceutically acceptable salt thereof, wherein L is

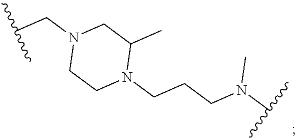 [SMILES: *CN1CCN(CCCN(*)C)C(C)C1]

wherein the left attachment point connects to LBM and the right attachment point connects to the —X— group of Formula A-I or —S(O)₂— group of Formula I.

Embodiment 302. The compound of any one of embodiments 1-280, or a pharmaceutically acceptable salt thereof, wherein L is

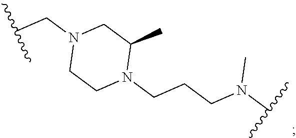 [SMILES: *CN1CCN(CCCN(*)C)[C@H](C)C1]

wherein the left attachment point connects to LBM and the right attachment point connects to the —X— group of Formula A-I or —S(O)₂— group of Formula I.

Embodiment 303. The compound of any one of embodiments 1-280, or a pharmaceutically acceptable salt thereof, wherein L is

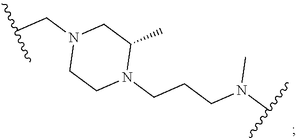 [SMILES: *CN1CCN(CCCN(*)C)[C@@H](C)C1]

wherein the left attachment point connects to LBM and the right attachment point connects to the —X— group of Formula A-I or —S(O)₂— group of Formula I.

Embodiment 304. The compound of any one of embodiments 1-280, or a pharmaceutically acceptable salt thereof, wherein L is

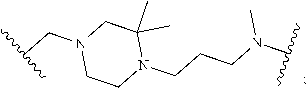 [SMILES: *CN1CCN(CCCN(*)C)C(C)(C)C1]

wherein the left attachment point connects to LBM and the right attachment point connects to the —X— group of Formula A-I or —S(O)₂— group of Formula I.

Embodiment 305. The compound of any one of embodiments 1-280, or a pharmaceutically acceptable salt thereof, wherein L is

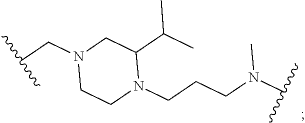 [SMILES: *CN1CCN(CCCN(*)C)C(C(C)C)C1]

wherein the left attachment point connects to LBM and the right attachment point connects to the —X— group of Formula A-I or —S(O)₂— group of Formula I.

Embodiment 306. The compound of any one of embodiments 1-280, or a pharmaceutically acceptable salt thereof, wherein L is

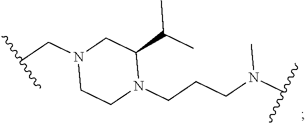 [SMILES: *CN1CCN(CCCN(*)C)[C@H](C(C)C)C1]

wherein the left attachment point connects to LBM and the right attachment point connects to the —X— group of Formula A-I or —S(O)₂— group of Formula I.

Embodiment 307. The compound of any one of embodiments 1-280, or a pharmaceutically acceptable salt thereof, wherein L is

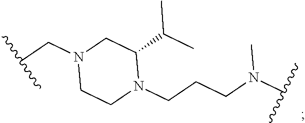 [SMILES: *CN1CCN(CCCN(*)C)[C@@H](C(C)C)C1]

wherein the left attachment point connects to LBM and the right attachment point connects to the —X— group of Formula A-I or —S(O)₂— group of Formula I.

Embodiment 308. The compound of any one of embodiments 1-280, or a pharmaceutically acceptable salt thereof, wherein L is

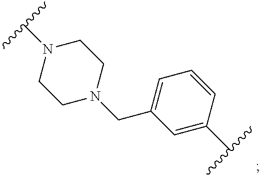 [SMILES: *c1cccc(CN2CCN(*)CC2)c1]

wherein the left attachment point connects to LBM and the right attachment point connects to the —X— group of Formula A-I or —S(O)₂— group of Formula I.

Embodiment 309. The compound of any one of embodiments 1-280, or a pharmaceutically acceptable salt thereof, wherein L is

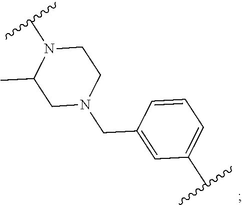 [SMILES: *c1cccc(CN2CCN(*)C(C)C2)c1]

wherein the left attachment point connects to LBM and the right attachment point connects to the —X— group of Formula A-I or —S(O)₂— group of Formula I.

Embodiment 310. The compound of any one of embodiments 1-280, or a pharmaceutically acceptable salt thereof, wherein L is

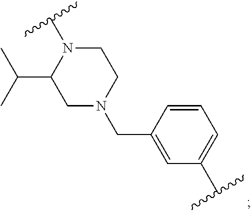 [SMILES: *c1cccc(CN2CCN(*)C(C(C)C)C2)c1]

wherein the left attachment point connects to LBM and the right attachment point connects to the —X— group of Formula A-I or —S(O)₂— group of Formula I.

Embodiment 311. The compound of any one of embodiments 1-280, or a pharmaceutically acceptable salt thereof, wherein L is

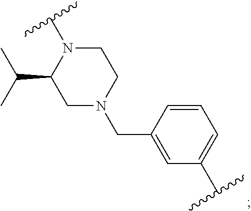 [SMILES: *c1cccc(CN2CCN(*)[C@H](C(C)C)C2)c1]

wherein the left attachment point connects to LBM and the right attachment point connects to the —X— group of Formula A-I or —S(O)₂— group of Formula I.

Embodiment 312. The compound of any one of embodiments 1-280, or a pharmaceutically acceptable salt thereof, wherein L is

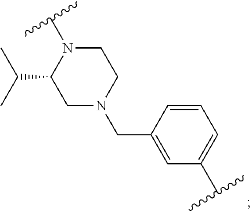 [SMILES: *c1cccc(CN2CCN(*)[C@@H](C(C)C)C2)c1]

wherein the left attachment point connects to LBM and the right attachment point connects to the —X— group of Formula A-I or —S(O)₂— group of Formula I.

Embodiment 313. The compound of any one of embodiments 1-280, or a pharmaceutically acceptable salt thereof, wherein L is

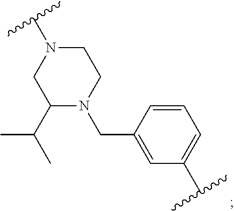 [SMILES: *c1cccc(CN2CCN(*)CC2C(C)C)c1]

wherein the left attachment point connects to LBM and the right attachment point connects to the —X— group of Formula A-I or —S(O)₂— group of Formula I.

Embodiment 314. The compound of any one of embodiments 1-280, or a pharmaceutically acceptable salt thereof, wherein L is

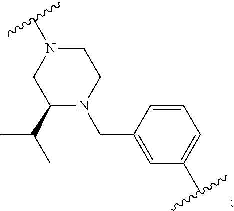 [SMILES: *c1cccc(CN2CCN(*)C[C@@H]2C(C)C)c1]

wherein the left attachment point connects to LBM and the right attachment point connects to the —X— group of Formula A-I or —S(O)₂— group of Formula I.

Embodiment 315. The compound of any one of embodiments 1-280, or a pharmaceutically acceptable salt thereof, wherein L is

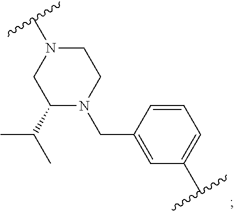 [SMILES: *c1cccc(CN2CCN(*)C[C@H]2C(C)C)c1]

wherein the left attachment point connects to LBM and the right attachment point connects to the —X— group of Formula A-I or —S(O)₂— group of Formula I.

Embodiment 316. The compound of any one of embodiments 1-280, or a pharmaceutically acceptable salt thereof, wherein L is

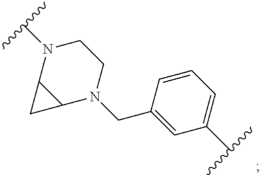 [SMILES: *c1cccc(CN2CCN(*)C3CC32)c1]

wherein the left attachment point connects to LBM and the right attachment point connects to the —X— group of Formula A-I or —S(O)₂— group of Formula I.

Embodiment 317. The compound of any one of embodiments 1-280, or a pharmaceutically acceptable salt thereof, wherein L is

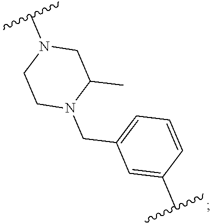 [SMILES: *c1cccc(CN2CCN(*)CC2C)c1]

wherein the left attachment point connects to LBM and the right attachment point connects to the —X— group of Formula A-I or —S(O)₂— group of Formula I.

Embodiment 318. The compound of any one of embodiments 1-280, or a pharmaceutically acceptable salt thereof, wherein L is

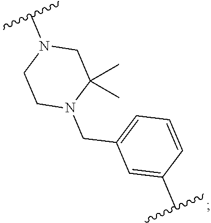 [SMILES: *c1cccc(CN2CCN(*)CC2(C)C)c1]

wherein the left attachment point connects to LBM and the right attachment point connects to the —X— group of Formula A-I or —S(O)₂— group of Formula I.

Embodiment 319. The compound of any one of embodiments 1-280, or a pharmaceutically acceptable salt thereof, wherein L is

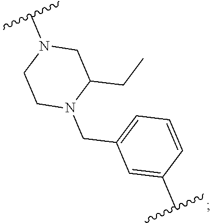 [SMILES: *c1cccc(CN2CCN(*)CC2CC)c1]

wherein the left attachment point connects to LBM and the right attachment point connects to the —X— group of Formula A-I or —S(O)₂— group of Formula I.

Embodiment 320. The compound of any one of embodiments 1-280, or a pharmaceutically acceptable salt thereof, wherein L is

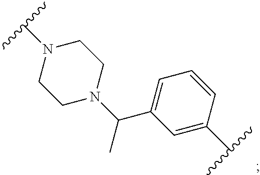 [SMILES: *c1cccc(C(C)N2CCN(*)CC2)c1]

wherein the left attachment point connects to LBM and the right attachment point connects to the —X— group of Formula A-I or —S(O)₂— group of Formula I.

Embodiment 321. The compound of any one of embodiments 1-280, or a pharmaceutically acceptable salt thereof, wherein L is

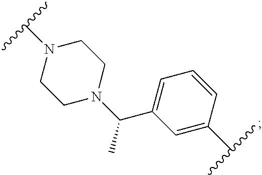 [SMILES: *c1cccc([C@H](C)N2CCN(*)CC2)c1]

wherein the left attachment point connects to LBM and the right attachment point connects to the —X— group of Formula A-I or —S(O)₂— group of Formula I.

Embodiment 322. The compound of any one of embodiments 1-280, or a pharmaceutically acceptable salt thereof, wherein L is

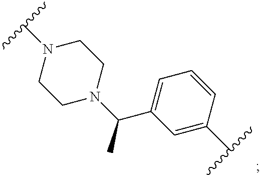 [SMILES: *c1cccc([C@@H](C)N2CCN(*)CC2)c1]

wherein the left attachment point connects to LBM and the right attachment point connects to the —X— group of Formula A-I or —S(O)₂— group of Formula I.

Embodiment 323. The compound of any one of embodiments 1-280, or a pharmaceutically acceptable salt thereof, wherein L is

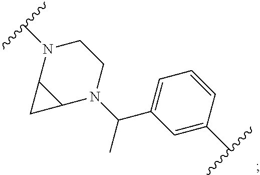 [SMILES: *c1cccc(C(C)N2CCN(*)C3CC32)c1]

wherein the left attachment point connects to LBM and the right attachment point connects to the —X— group of Formula A-I or —S(O)₂— group of Formula I.

Embodiment 324. The compound of any one of embodiments 1-280, or a pharmaceutically acceptable salt thereof, wherein L is

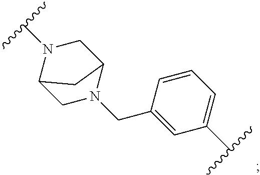 [SMILES: *c1cccc(CN2CC3CC2CN3*)c1]

wherein the left attachment point connects to LBM and the right attachment point connects to the —X— group of Formula A-I or —S(O)₂— group of Formula I.

Embodiment 325. The compound of any one of embodiments 1-280, or a pharmaceutically acceptable salt thereof, wherein L is

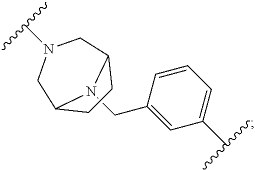 [SMILES: *c1cccc(CN2C3CCC2CN(*)C3)c1]

wherein the left attachment point connects to LBM and the right attachment point connects to the —X— group of Formula A-I or —S(O)₂— group of Formula I.

Embodiment 326. The compound of any one of embodiments 1-280, or a pharmaceutically acceptable salt thereof, wherein L is

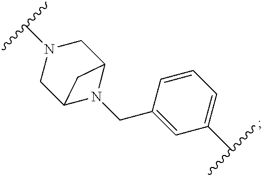 [SMILES: *c1cccc(CN2C3CC2CN(*)C3)c1]

wherein the left attachment point connects to LBM and the right attachment point connects to the —X— group of Formula A-I or —S(O)₂— group of Formula I.

Embodiment 327. The compound of any one of embodiments 1-280, or a pharmaceutically acceptable salt thereof, wherein L is

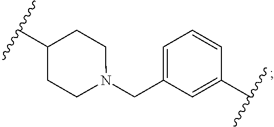 [SMILES: *c1cccc(CN2CCC(*)CC2)c1]

wherein the left attachment point connects to LBM and the right attachment point connects to the —X— group of Formula A-I or —S(O)₂— group of Formula I.

Embodiment 328. The compound of any one of embodiments 1-280, or a pharmaceutically acceptable salt thereof, wherein L is

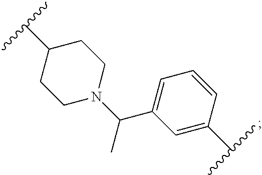 [SMILES: *c1cccc(C(C)N2CCC(*)CC2)c1]

wherein the left attachment point connects to LBM and the right attachment point connects to the —X— group of Formula A-I or —S(O)₂— group of Formula I.

Embodiment 329. The compound of any one of embodiments 1-280, or a pharmaceutically acceptable salt thereof, wherein L is

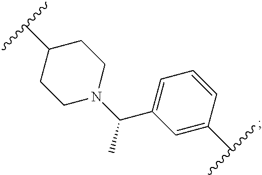 [SMILES: *c1cccc([C@H](C)N2CCC(*)CC2)c1]

wherein the left attachment point connects to LBM and the right attachment point connects to the —X— group of Formula A-I or —S(O)₂— group of Formula I.

Embodiment 330. The compound of any one of embodiments 1-280, or a pharmaceutically acceptable salt thereof, wherein L is

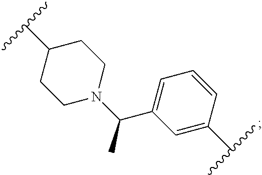 [SMILES: *c1cccc([C@@H](C)N2CCC(*)CC2)c1]

wherein the left attachment point connects to LBM and the right attachment point connects to the —X— group of Formula A-I or —S(O)₂— group of Formula I.

Embodiment 331. The compound of any one of embodiments 1-280, or a pharmaceutically acceptable salt thereof, wherein L is

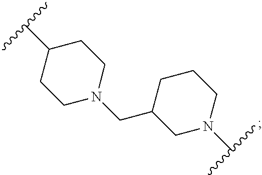 [SMILES: *C1CCN(CC2CCCN(*)C2)CC1]

wherein the left attachment point connects to LBM and the right attachment point connects to the —X— group of Formula A-I or —S(O)₂— group of Formula I.

Embodiment 332. The compound of any one of embodiments 1-280, or a pharmaceutically acceptable salt thereof, wherein L is

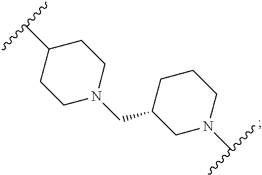 [SMILES: *C1CCN(C[C@@H]2CCCN(*)C2)CC1]

wherein the left attachment point connects to LBM and the right attachment point connects to the —X— group of Formula A-I or —S(O)₂— group of Formula I.

Embodiment 333. The compound of any one of embodiments 1-280, or a pharmaceutically acceptable salt thereof, wherein L is

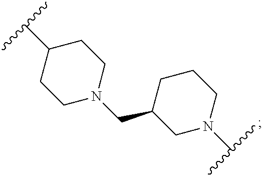 [SMILES: *C1CCN(C[C@H]2CCCN(*)C2)CC1]

wherein the left attachment point connects to LBM and the right attachment point connects to the —X— group of Formula A-I or —S(O)₂— group of Formula I.

Embodiment 334. The compound of any one of embodiments 1-280, or a pharmaceutically acceptable salt thereof, wherein L is

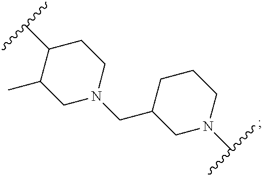 [SMILES: *C1CCN(CC2CCCN(*)C2)CC1C]

wherein the left attachment point connects to LBM and the right attachment point connects to the —X— group of Formula A-I or —S(O)₂— group of Formula I.

Embodiment 335. The compound of any one of embodiments 1-280, or a pharmaceutically acceptable salt thereof, wherein L is

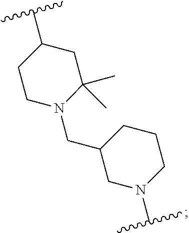 [SMILES: *C1CCN(CC2CCCN(*)C2)C(C)(C)C1]

wherein the left attachment point connects to LBM and the right attachment point connects to the —X— group of Formula A-I or —S(O)₂— group of Formula I.

Embodiment 336. The compound of any one of embodiments 1-280, or a pharmaceutically acceptable salt thereof, wherein L is

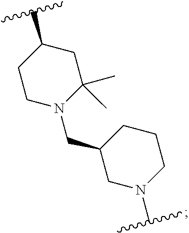 [SMILES: *[C@@H]1CCN(C[C@H]2CCCN(*)C2)C(C)(C)C1]

wherein the left attachment point connects to LBM and the right attachment point connects to the —X— group of Formula A-I or —S(O)₂— group of Formula I.

Embodiment 337. The compound of any one of embodiments 1-280, or a pharmaceutically acceptable salt thereof, wherein L is

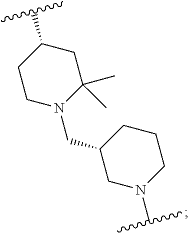 [SMILES: *[C@H]1CCN(C[C@@H]2CCCN(*)C2)C(C)(C)C1]

wherein the left attachment point connects to LBM and the right attachment point connects to the —X— group of Formula A-I or —S(O)₂— group of Formula I.

Embodiment 338. The compound of any one of embodiments 1-280, or a pharmaceutically acceptable salt thereof, wherein L is

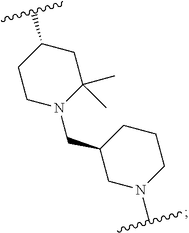 [SMILES: *[C@H]1CCN(C[C@H]2CCCN(*)C2)C(C)(C)C1]

wherein the left attachment point connects to LBM and the right attachment point connects to the —X— group of Formula A-I or —S(O)₂— group of Formula I.

Embodiment 339. The compound of any one of embodiments 1-280, or a pharmaceutically acceptable salt thereof, wherein L is

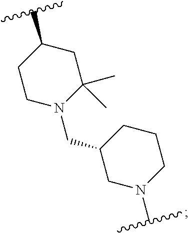 [SMILES: *[C@@H]1CCN(C[C@@H]2CCCN(*)C2)C(C)(C)C1]

wherein the left attachment point connects to LBM and the right attachment point connects to the —X— group of Formula A-I or —S(O)₂— group of Formula I.

Embodiment 340. The compound of any one of embodiments 1-280, or a pharmaceutically acceptable salt thereof, wherein L is

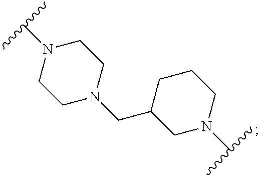 [SMILES: *N1CCN(CC2CCCN(*)C2)CC1]

wherein the left attachment point connects to LBM and the right attachment point connects to the —X— group of Formula A-I or —S(O)₂— group of Formula I.

Embodiment 341. The compound of any one of embodiments 1-280, or a pharmaceutically acceptable salt thereof, wherein L is

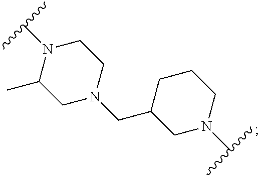 [SMILES: *N1CCCC(CN2CCN(*)C(C)C2)C1]

wherein the left attachment point connects to LBM and the right attachment point connects to the —X— group of Formula A-I or —S(O)₂— group of Formula I.

Embodiment 342. The compound of any one of embodiments 1-280, or a pharmaceutically acceptable salt thereof, wherein L is

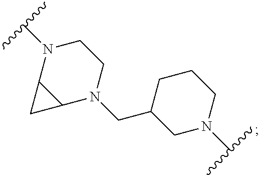 [SMILES: *N1CCCC(CN2CCN(*)C3CC32)C1]

wherein the left attachment point connects to LBM and the right attachment point connects to the —X— group of Formula A-I or —S(O)₂— group of Formula I.

Embodiment 343. The compound of any one of embodiments 1-280, or a pharmaceutically acceptable salt thereof, wherein L is

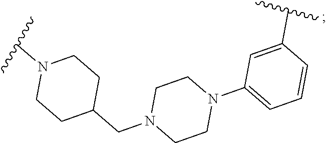 [SMILES: *c1cccc(N2CCN(CC3CCN(*)CC3)CC2)c1]

wherein the left attachment point connects to LBM and the right attachment point connects to the —X— group of Formula A-I or —S(O)₂— group of Formula I.

Embodiment 344. The compound of any one of embodiments 1-280, or a pharmaceutically acceptable salt thereof, wherein L is

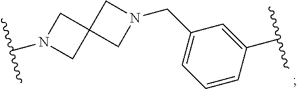 [SMILES: *c1cccc(CN2CC3(CN(*)C3)C2)c1]

wherein the left attachment point connects to LBM and the right attachment point connects to the —X— group of Formula A-I or —S(O)₂— group of Formula I.

Embodiment 345. The compound of any one of embodiments 1-280, or a pharmaceutically acceptable salt thereof, wherein L is

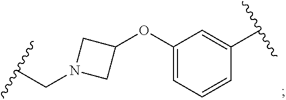 [SMILES: *CN1CC(Oc2cccc(*)c2)C1]

wherein the left attachment point connects to LBM and the right attachment point connects to the —X— group of Formula A-I or —S(O)₂— group of Formula I.

Embodiment 346. The compound of any one of embodiments 1-280, or a pharmaceutically acceptable salt thereof, wherein L is

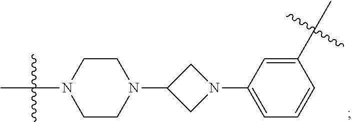 [SMILES: *c1cccc(N2CC(N3CCN(*)CC3)C2)c1]

wherein the left attachment point connects to LBM and the right attachment point connects to the —X— group of Formula A-I or —S(O)₂— group of Formula I.

Embodiment 347. The compound of any one of embodiments 1-280, or a pharmaceutically acceptable salt thereof, wherein L is

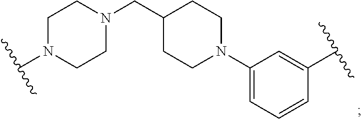 [SMILES: *c1cccc(N2CCC(CN3CCN(*)CC3)CC2)c1]

wherein the left attachment point connects to LBM and the right attachment point connects to the —X— group of Formula A-I or —S(O)₂— group of Formula I.

Embodiment 348. The compound of any one of embodiments 1-280, or a pharmaceutically acceptable salt thereof, wherein L is

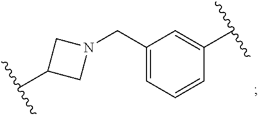 [SMILES: *c1cccc(CN2CC(*)C2)c1]

wherein the left attachment point connects to LBM and the right attachment point connects to the —X— group of Formula A-I or —S(O)₂— group of Formula I.

Embodiment 349. The compound of any one of embodiments 1-280, or a pharmaceutically acceptable salt thereof, wherein L is

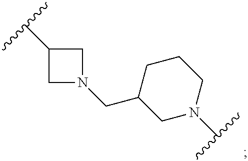 [SMILES: *C1CN(CC2CCCN(*)C2)C1]

wherein the left attachment point connects to LBM and the right attachment point connects to the —X— group of Formula A-I or —S(O)₂— group of Formula I.

Embodiment 350. The compound of any one of embodiments 1-280, or a pharmaceutically acceptable salt thereof, wherein L is

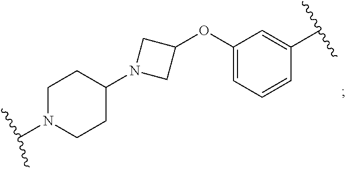 [SMILES: *c1cccc(OC2CN(C3CCN(*)CC3)C2)c1]

wherein the left attachment point connects to LBM and the right attachment point connects to the —X— group of Formula A-I or —S(O)₂— group of Formula I.

Embodiment 351. The compound of any one of embodiments 1-280, or a pharmaceutically acceptable salt thereof, wherein L is

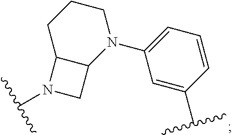 [SMILES: *c1cccc(N2CCCC3C2CN3*)c1]

wherein the left attachment point connects to LBM and the right attachment point connects to the —X— group of Formula A-I or —S(O)₂— group of Formula I.

Embodiment 352. The compound of any one of embodiments 1-280, or a pharmaceutically acceptable salt thereof, wherein L is

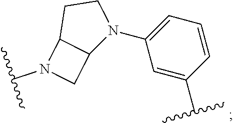 [SMILES: *c1cccc(N2CCC3C2CN3*)c1]

wherein the left attachment point connects to LBM and the right attachment point connects to the —X— group of Formula A-I or —S(O)₂— group of Formula I.

Embodiment 353. The compound of any one of embodiments 1-280, or a pharmaceutically acceptable salt thereof, wherein L is

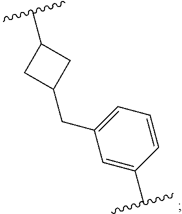 [SMILES: *c1cccc(CC2CC(*)C2)c1]

wherein the left attachment point connects to LBM and the right attachment point connects to the —X— group of Formula A-I or —S(O)₂— group of Formula I.

Embodiment 354. The compound of any one of embodiments 1-280, or a pharmaceutically acceptable salt thereof, wherein L is

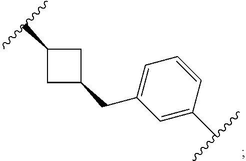 [SMILES: *c1cccc(C[C@H]2C[C@@H](*)C2)c1]

wherein the left attachment point connects to LBM and the right attachment point connects to the —X— group of Formula A-I or —S(O)₂— group of Formula I.

Embodiment 355. The compound of any one of embodiments 1-280, or a pharmaceutically acceptable salt thereof, wherein L is

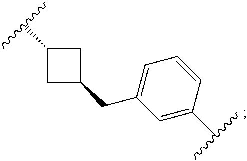 [SMILES: *c1cccc(C[C@H]2C[C@H](*)C2)c1]

wherein the left attachment point connects to LBM and the right attachment point connects to the —X— group of Formula A-I or —S(O)₂— group of Formula I.

Embodiment 356. The compound of any one of embodiments 1-280, or a pharmaceutically acceptable salt thereof, wherein L

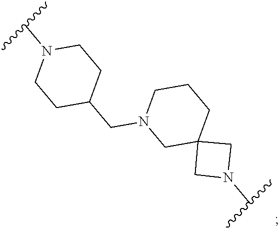 [SMILES: *N1CCC(CN2CCCC3(CN(*)C3)C2)CC1]

is wherein the left attachment point connects to LBM and the right attachment point connects to the —X— group of Formula A-I or —S(O)₂— group of Formula I.

Embodiment 357. The compound of any one of embodiments 1-280, or a pharmaceutically acceptable salt thereof, wherein L is

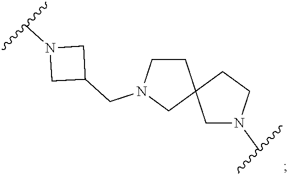 [SMILES: *N1CC(CN2CCC3(CCN(*)C3)C2)C1]

wherein the left attachment point connects to LBM and the right attachment point connects to the —X— group of Formula A-I or —S(O)₂— group of Formula I.

Embodiment 358. The compound of any one of embodiments 1-280, or a pharmaceutically acceptable salt thereof, wherein L is

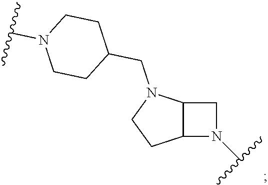 [SMILES: *N1CCC(CN2CCC3C2CN3*)CC1]

wherein the left attachment point connects to LBM and the right attachment point connects to the —X— group of Formula A-I or —S(O)₂— group of Formula I.

Embodiment 359. The compound of any one of embodiments 1-280, or a pharmaceutically acceptable salt thereof, wherein L is

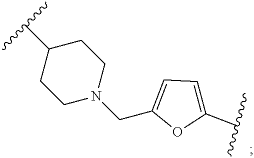 [SMILES: *c1ccc(CN2CCC(*)CC2)o1]

wherein the left attachment point connects to LBM and the right attachment point connects to the —X— group of Formula A-I or —S(O)₂— group of Formula I.

Embodiment 360. The compound of any one of embodiments 1-280, or a pharmaceutically acceptable salt thereof, wherein L is

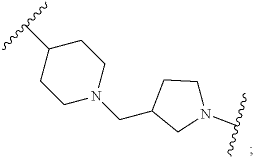 [SMILES: *C1CCN(CC2CCN(*)C2)CC1]

wherein the left attachment point connects to LBM and the right attachment point connects to the —X— group of Formula A-I or —S(O)₂— group of Formula I.

Embodiment 361. The compound of any one of embodiments 1-280, or a pharmaceutically acceptable salt thereof, wherein L is

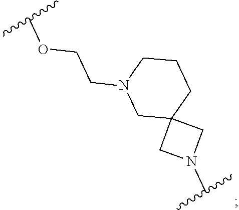 [SMILES: *OCCN1CCCC2(CN(*)C2)C1]

wherein the left attachment point connects to LBM and the right attachment point connects to the —X— group of Formula A-I or —S(O)₂— group of Formula I.

Embodiment 362. The compound of any one of embodiments 1-280, or a pharmaceutically acceptable salt thereof, wherein L is

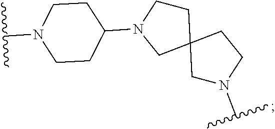 [SMILES: *N1CCC(N2CCC3(CCN(*)C3)C2)CC1]

wherein the left attachment point connects to LBM and the right attachment point connects to the —X— group of Formula A-I or —S(O)₂— group of Formula I.

Embodiment 363. The compound of any one of embodiments 1-280, or a pharmaceutically acceptable salt thereof, wherein L is

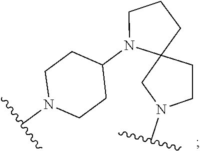 [SMILES: *N1CCC(N2CCCC23CCN(*)C3)CC1]

wherein the left attachment point connects to LBM and the right attachment point connects to the —X— group of Formula A-I or —S(O)₂— group of Formula I.

Embodiment 364. The compound of any one of embodiments 1-280, or a pharmaceutically acceptable salt thereof, wherein L is

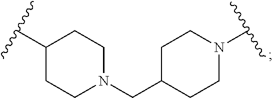 [SMILES: *C1CCN(CC2CCN(*)CC2)CC1]

wherein the left attachment point connects to LBM and the right attachment point connects to the —X— group of Formula A-I or —S(O)₂— group of Formula I.

Embodiment 365. The compound of any one of embodiments 1-280, or a pharmaceutically acceptable salt thereof, wherein L is

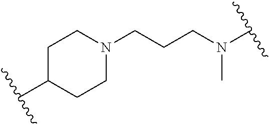 [SMILES: *C1CCN(CCCN(*)C)CC1]

wherein the left attachment point connects to LBM and the right attachment point connects to the —X— group of Formula A-I or —S(O)₂— group of Formula I.

Embodiment 366. The compound of any one of embodiments 1-365, or a pharmaceutically acceptable salt thereof, wherein each R⁴ is independently selected from —D, —Me, —Et, —F, —Cl and —OH.

Embodiment 367. The compound of any one of embodiments 1-365, or a pharmaceutically acceptable salt thereof, wherein each R⁴ is independently selected from —Me and —F.

Embodiment 368. The compound of any one of embodiments 1-365, or a pharmaceutically acceptable salt thereof, wherein each R⁴ is independently —Me.

Embodiment 369. The compound of any one of embodiments 1-365, or a pharmaceutically acceptable salt thereof, wherein each R⁴ is independently —F.

Embodiment 370. The compound of any one of embodiments 1-369, or a pharmaceutically acceptable salt thereof, wherein each R⁵ is independently selected from —Me, —Et, —F, —Cl and —OH.

Embodiment 371. The compound of any one of embodiments 1-369, or a pharmaceutically acceptable salt thereof, wherein each R⁵ is independently —Me.

Embodiment 372. The compound of any one of embodiments 1-371, or a pharmaceutically acceptable salt thereof, wherein r is 0, 1 or 2.

Embodiment 373. The compound of any one of embodiments 1-371, or a pharmaceutically acceptable salt thereof, wherein r is 0.

Embodiment 374. The compound of any one of embodiments 1-371, or a pharmaceutically acceptable salt thereof, wherein r is 1.

Embodiment 375. The compound of any one of embodiments 1-371, or a pharmaceutically acceptable salt thereof, wherein r is 2.

Embodiment 376. The compound of any one of embodiments 1-375, or a pharmaceutically acceptable salt thereof, wherein s is 0, 1 or 2.

Embodiment 377. The compound of any one of embodiments 1-371, or a pharmaceutically acceptable salt thereof, wherein s is 0.

Embodiment 378. The compound of any one of embodiments 1-371, or a pharmaceutically acceptable salt thereof, wherein s is 1.

Embodiment 379. The compound of any one of embodiments 1-371, or a pharmaceutically acceptable salt thereof, wherein s is 2.

Embodiment 380. The compound of any one of embodiments 1-379, or a pharmaceutically acceptable salt thereof, wherein Y^{a} is —CH—.

Embodiment 381. The compound of any one of embodiments 1-379, or a pharmaceutically acceptable salt thereof, wherein Y^{a} is —N—.

Embodiment 382. The compound of any one of embodiments 1-381, or a pharmaceutically acceptable salt thereof, wherein Z^{a} is selected from the group consisting of a bond, —NH—, —O— and —NHC(O)—.

Embodiment 383. The compound of any one of embodiments 1-381, or a pharmaceutically acceptable salt thereof, wherein Z^{a} is selected from the group consisting of a bond, —NH— and —NHC(O)—.

Embodiment 384. The compound of any one of embodiments 1-381, or a pharmaceutically acceptable salt thereof, wherein Z^{a} is selected from the group consisting of —NH—, and —NHC(O)—.

Embodiment 385. The compound of any one of embodiments 1-381, or a pharmaceutically acceptable salt thereof, wherein Z^{a} is a bond.

Embodiment 386. The compound of any one of embodiments 1-381, or a pharmaceutically acceptable salt thereof, wherein Z^{a} is —NH—.

Embodiment 387. The compound of any one of embodiments 1-381, or a pharmaceutically acceptable salt thereof, wherein Z^{a} is —O—.

Embodiment 388. The compound of any one of embodiments 1-381, or a pharmaceutically acceptable salt thereof, wherein Z^{a} is —NHC(O)—.

Embodiment 389. The compound of any one of embodiments 1-388, or a pharmaceutically acceptable salt thereof, wherein Ring B is phenylene.

Embodiment 390. The compound of any one of embodiments 1-388, or a pharmaceutically acceptable salt thereof, wherein Ring B is a 4-10 membered heterocyclylene containing 1, 2 or 3 heteroatoms selected from O, N and S and oxidized forms thereof.

Embodiment 391. The compound of any one of embodiments 1-388, or a pharmaceutically acceptable salt thereof, wherein Ring B is a 5-6-membered monocyclic heteroarylene containing 1, 2, 3 or 4 heteroatoms selected from O, N and S and oxidized forms thereof.

Embodiment 392. The compound of any one of embodiments 1-388, or a pharmaceutically acceptable salt thereof, wherein Ring B is a 9-10-membered fused bicyclic heteroarylene containing 1, 2, 3 or 4 heteroatoms selected from O, N and S and oxidized forms thereof.

Embodiment 393. The compound of any one of embodiments 1-379, or a pharmaceutically acceptable salt thereof, wherein LBM is

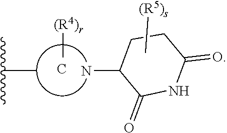

Embodiment 394. The compound of any one of embodiments 1-379 and 393, or a pharmaceutically acceptable salt thereof, wherein LBM is selected from the group consisting of:

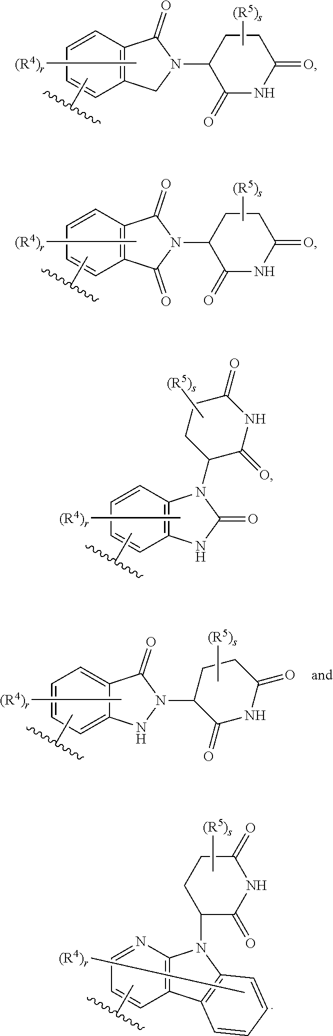

Embodiment 395. The compound of any one of embodiments 1-379 and 393, or a pharmaceutically acceptable salt thereof, wherein LBM is selected from the group consisting

of

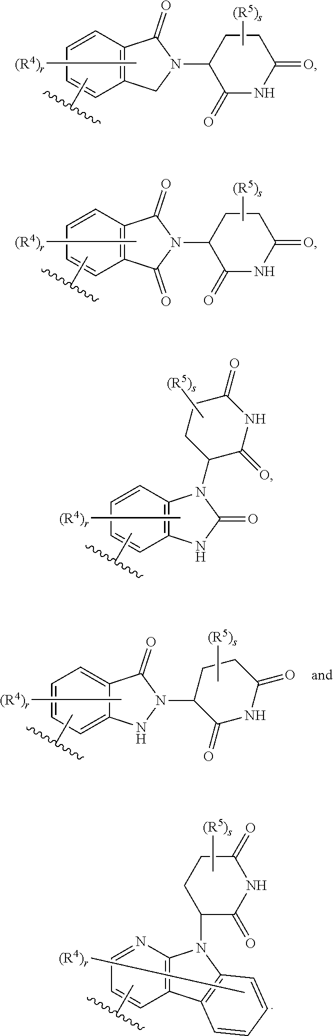

Embodiment 396. The compound of any one of embodiments 1-379 and 393, or a pharmaceutically acceptable salt thereof, wherein LBM is selected from the group consisting of:

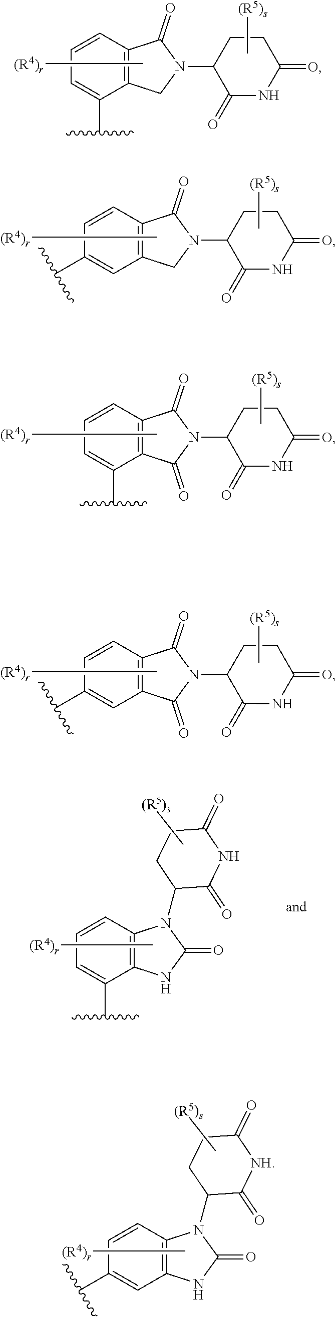

Embodiment 397. The compound of any one of embodiments 1-371 and 393, or a pharmaceutically acceptable salt thereof, wherein LBM is selected from the group consisting

of

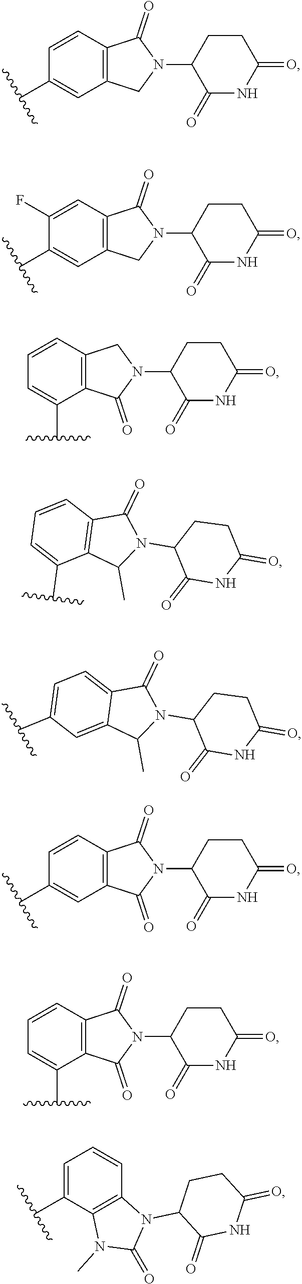

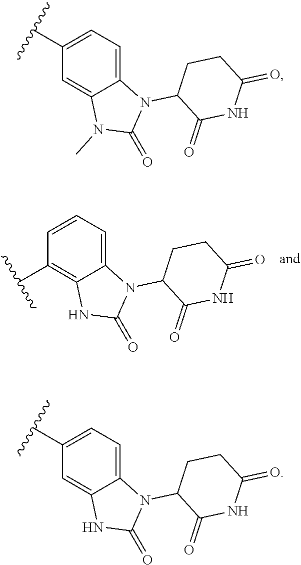 [SMILES: *c1ccc2c(c1)n(C)c(=O)n2C1CCC(=O)NC1=O ; *c1cccc2c1[nH]c(=O)n2C1CCC(=O)NC1=O ; *c1ccc2c(c1)[nH]c(=O)n2C1CCC(=O)NC1=O]

Embodiment 398. The compound of any one of embodiments 1-392, or a pharmaceutically acceptable salt thereof, wherein LBM is

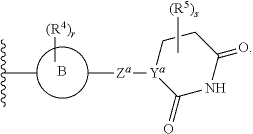

Embodiment 399. The compound of any one of embodiments 1-392 and 398, or a pharmaceutically acceptable salt thereof, wherein LBM is selected from the group consisting of:

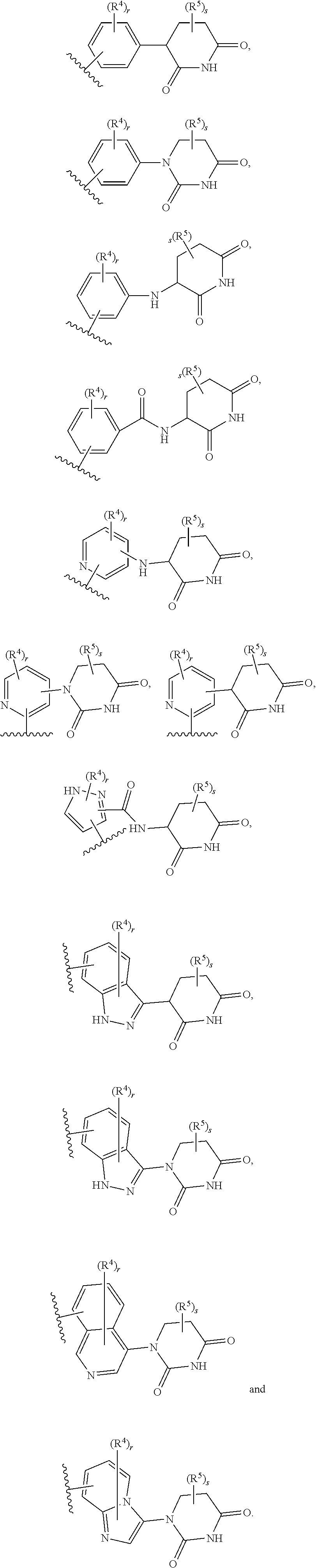

Embodiment 400. The compound of any one of embodiments 1-392 and 398, or a pharmaceutically acceptable salt thereof, wherein LBM is selected from the group consisting of:

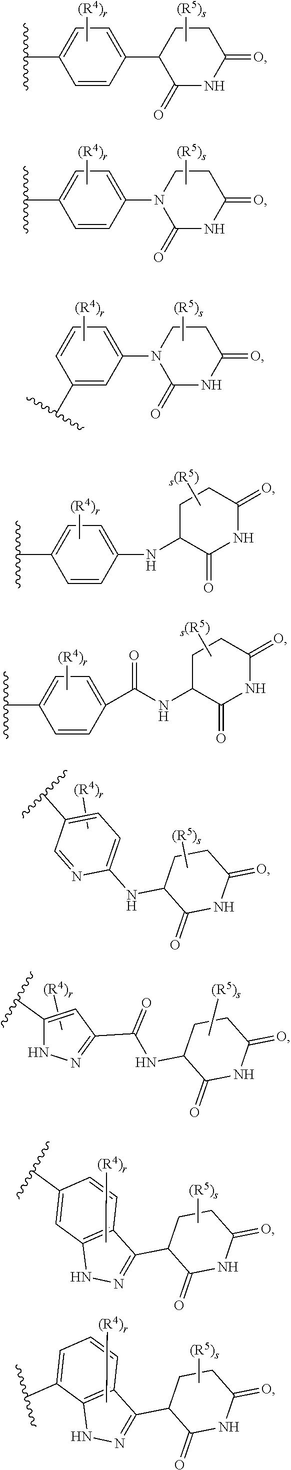

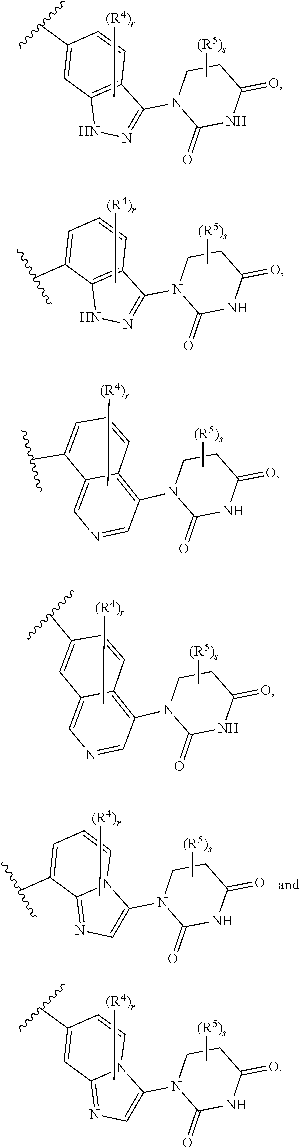

Embodiment 401. The compound of any one of embodiments 1-371 and 398, or a pharmaceutically acceptable salt thereof, wherein LBM is selected from the group consisting of:

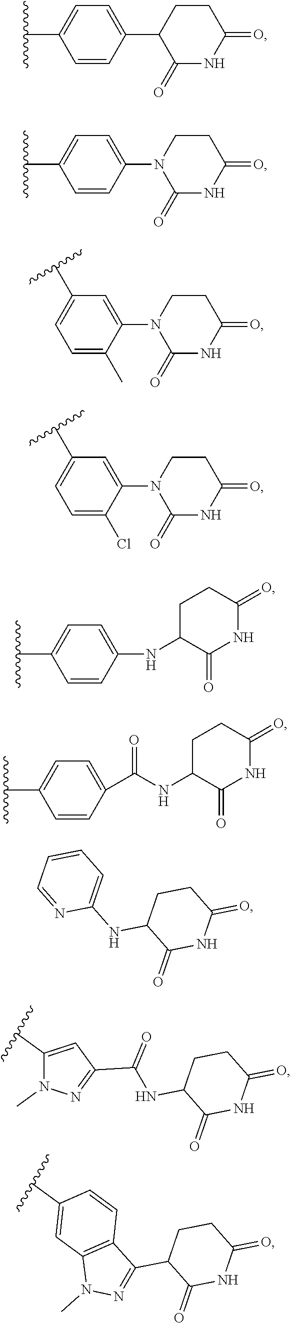

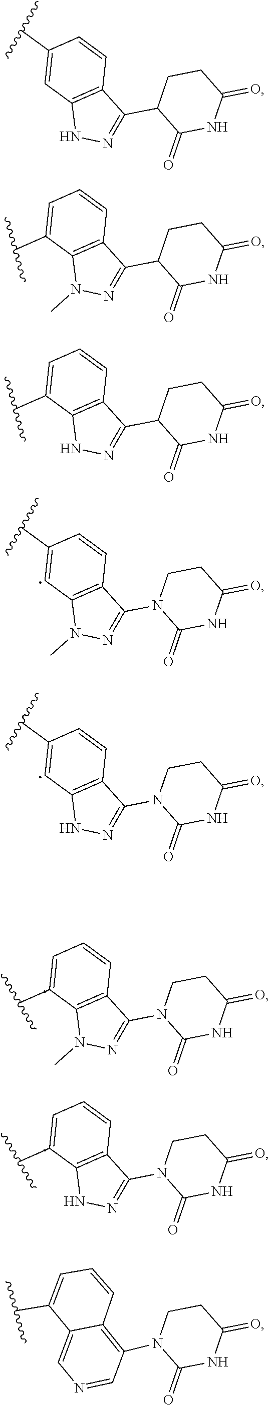

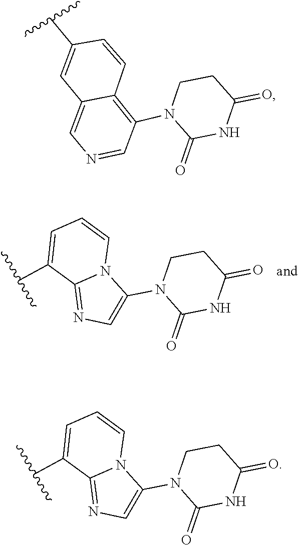 [SMILES: *c1ccc2c(N3CCC(=O)NC3=O)cncc2c1 ; *c1cccn2c(N3CCC(=O)NC3=O)cnc12 ; *c1cccn2c(N3CCC(=O)NC3=O)cnc12]

Embodiment 402. The compound of any one of embodiments 1-371 and 398, or a pharmaceutically acceptable salt thereof, wherein LBM is selected from the group consisting of:

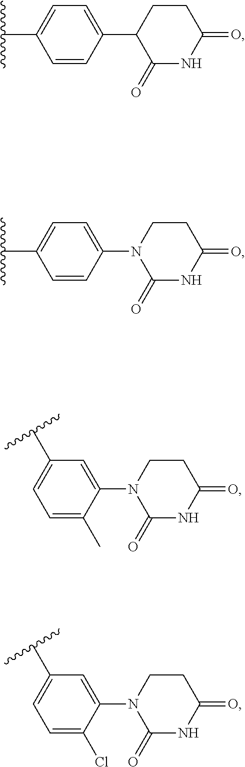 [SMILES: *c1ccc(C2CCC(=O)NC2=O)cc1 ; *c1ccc(N2CCC(=O)NC2=O)cc1 ; *c1ccc(C)c(N2CCC(=O)NC2=O)c1 ; *c1ccc(Cl)c(N2CCC(=O)NC2=O)c1]

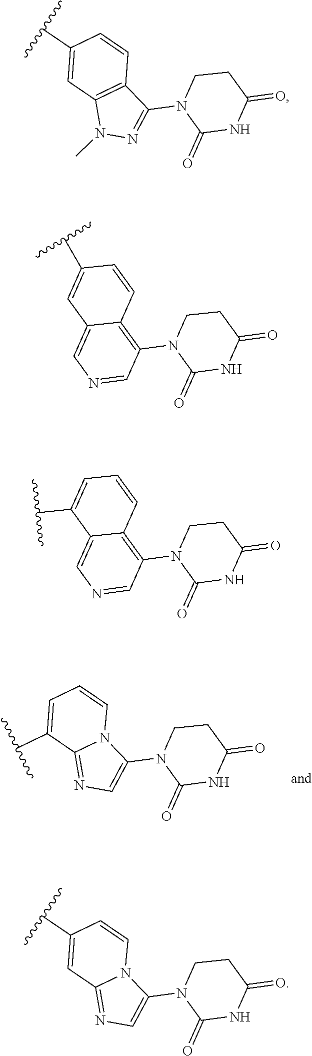 [SMILES: *c1ccc2c(N3CCC(=O)NC3=O)nn(C)c2c1 ; *c1ccc2c(N3CCC(=O)NC3=O)cncc2c1 ; *c1cccc2c(N3CCC(=O)NC3=O)cncc12 ; *c1cccn2c(N3CCC(=O)NC3=O)cnc12 ; *c1ccn2c(N3CCC(=O)NC3=O)cnc2c1]

Embodiment 403. The compound of any one of embodiments 1-379, or a pharmaceutically acceptable salt thereof, wherein LBM is selected from the group consisting of.

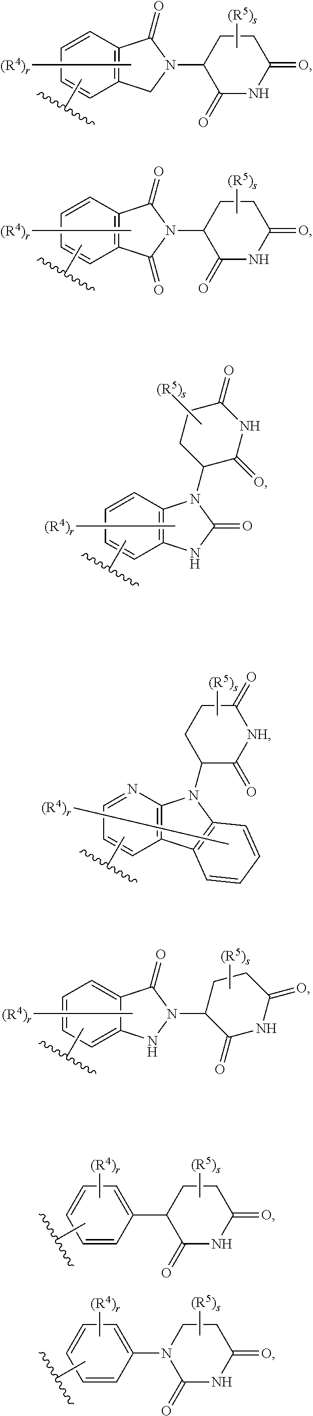

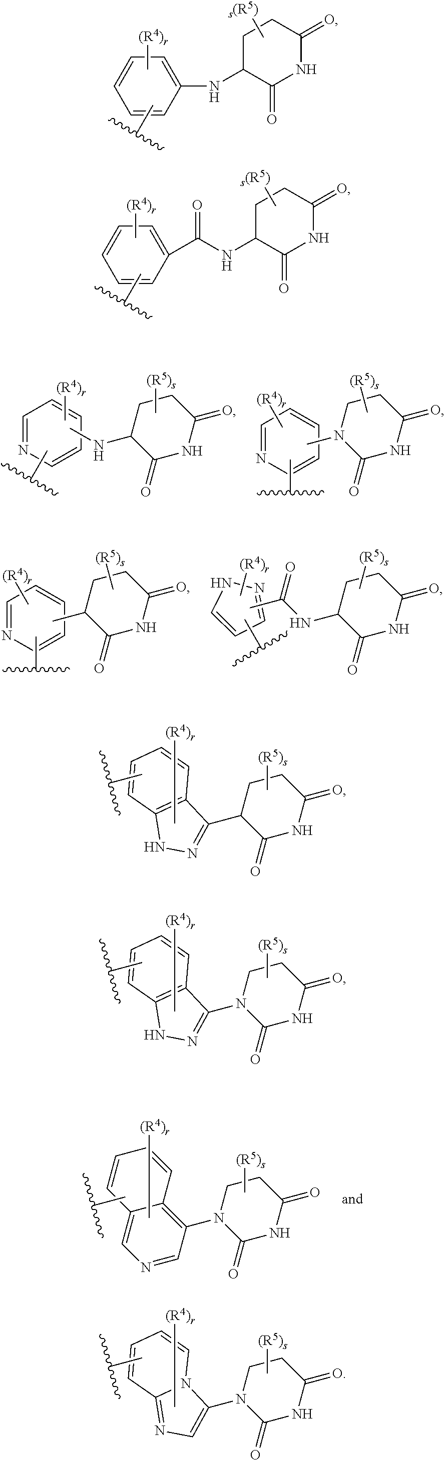

Embodiment 404. The compound of any one of embodiments 1-379, or a pharmaceutically acceptable salt thereof, wherein LBM is selected from:

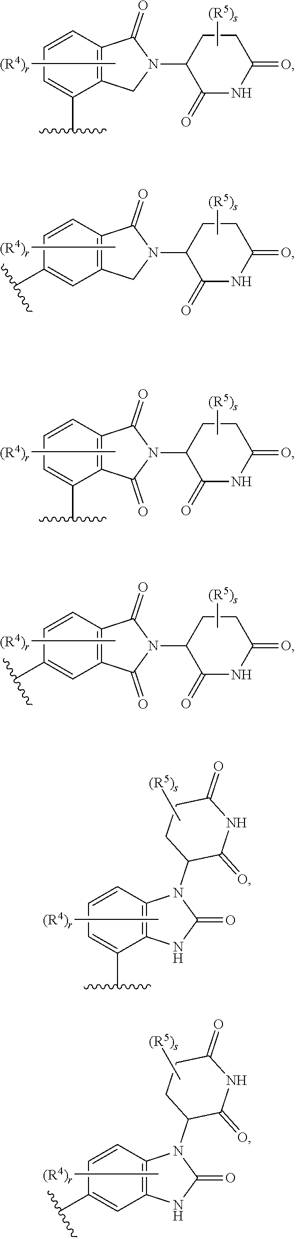

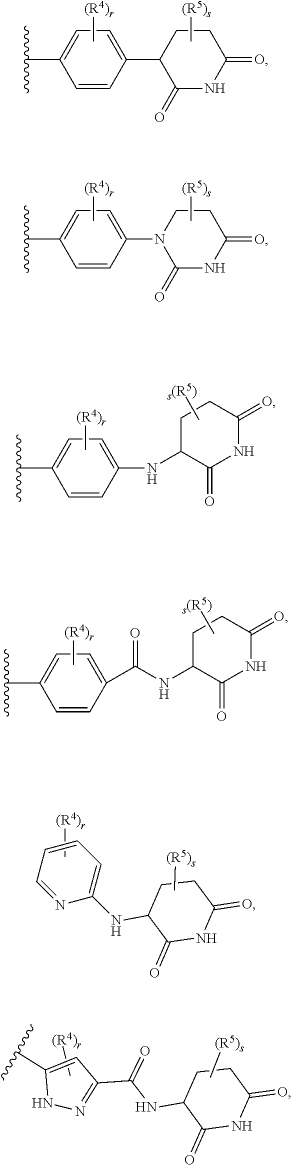

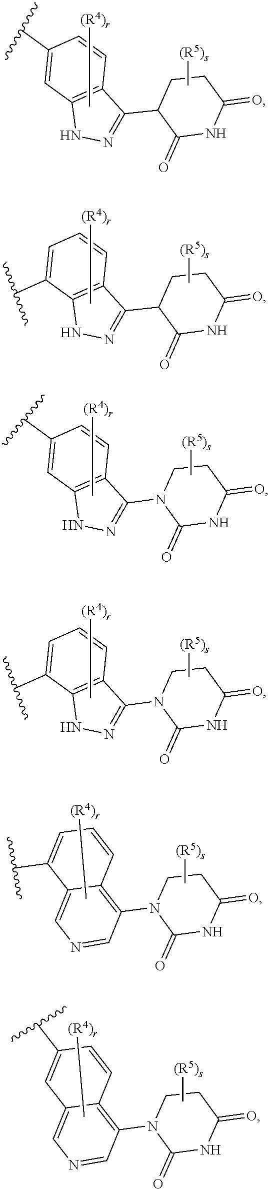

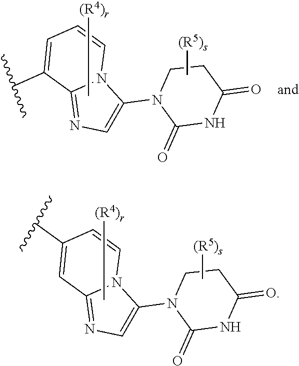

Embodiment 405. The compound of any one of embodiments 1-379, or a pharmaceutically acceptable salt thereof, wherein LBM is selected from:

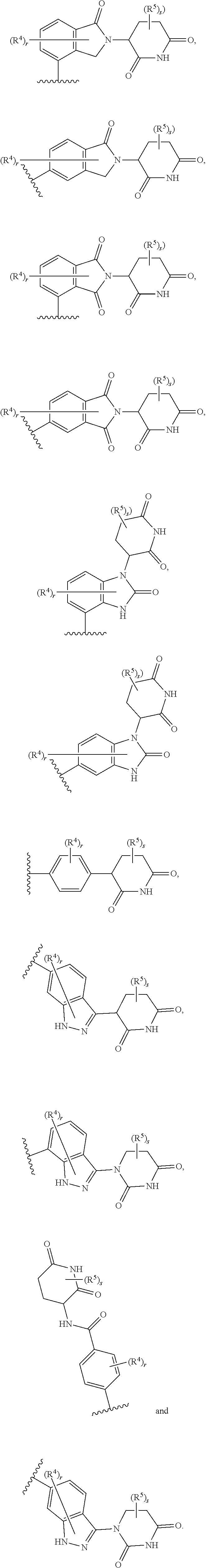

Embodiment 406. The compound of any one of embodiments 1-379, or a pharmaceutically acceptable salt thereof, wherein LBM is selected from:

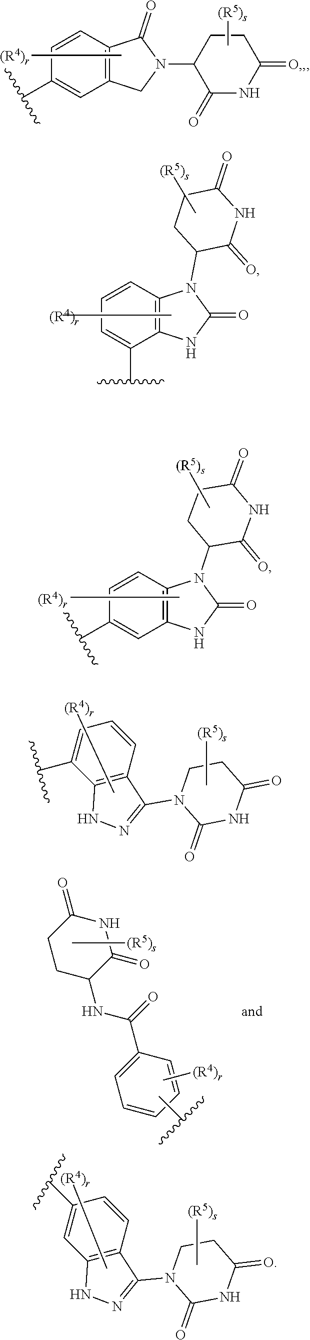

Embodiment 407. The compound of any one of embodiments 1-379, or a pharmaceutically acceptable salt thereof, wherein LBM is selected from:

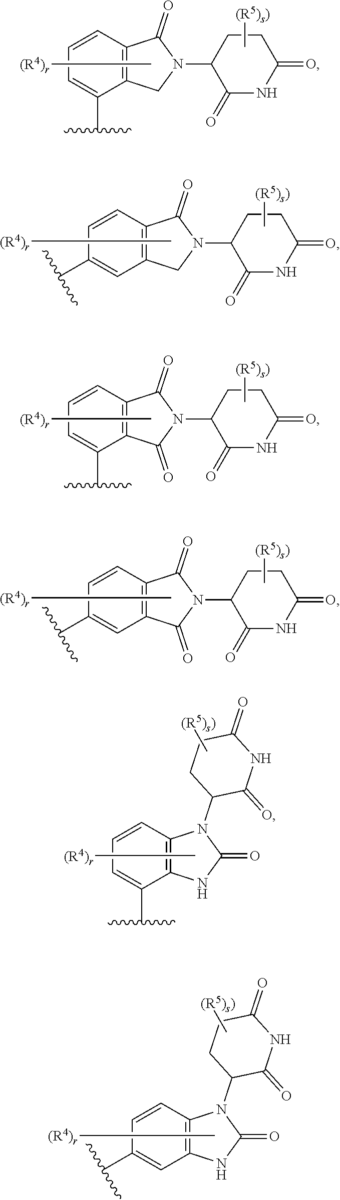

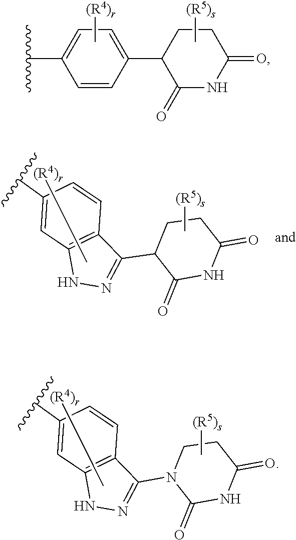

Embodiment 408. The compound of any one of embodiments 1-379, or a pharmaceutically acceptable salt thereof, wherein LBM is

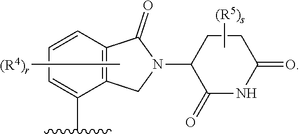

Embodiment 409. The compound of any one of embodiments 1-379, or a pharmaceutically acceptable salt thereof, wherein LBM is

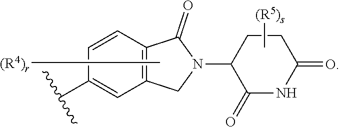

Embodiment 410. The compound of any one of embodiments 1-379, or a pharmaceutically acceptable salt thereof, wherein LBM is

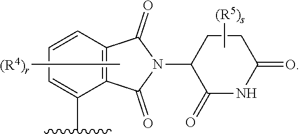

Embodiment 411. The compound of any one of embodiments 1-379, or a pharmaceutically acceptable salt thereof, wherein LBM is

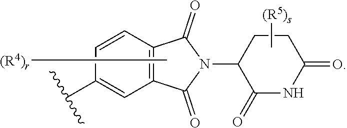

Embodiment 412. The compound of any one of embodiments 1-379, or a pharmaceutically acceptable salt thereof, wherein LBM is

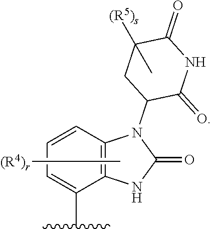

Embodiment 413. The compound of any one of embodiments 1-379, or a pharmaceutically acceptable salt thereof, wherein LBM is

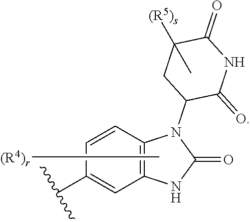

Embodiment 414. The compound of any one of embodiments 1-379, or a pharmaceutically acceptable salt thereof, wherein LBM is

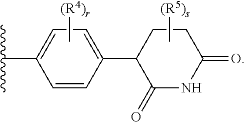

Embodiment 415. The compound of any one of embodiments 1-379, or a pharmaceutically acceptable salt thereof, wherein LBM is

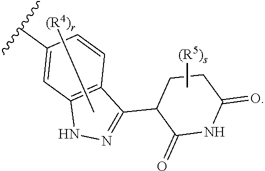

Embodiment 416. The compound of any one of embodiments 1-379, or a pharmaceutically acceptable salt thereof, wherein LBM is

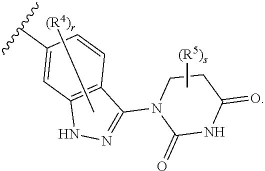

Embodiment 417. The compound of any one of embodiments 1-379, or a pharmaceutically acceptable salt thereof, wherein LBM is

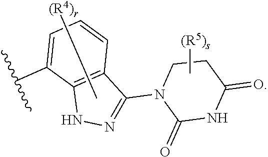

Embodiment 418. The compound of any one of embodiments 1-379, or a pharmaceutically acceptable salt thereof, wherein LBM is

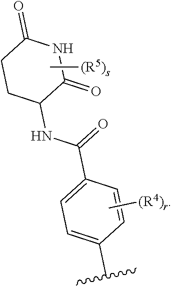

Embodiment 419. The compound of any one of embodiments 1-365, or a pharmaceutically acceptable salt thereof, wherein LBM is selected from the group consisting of:

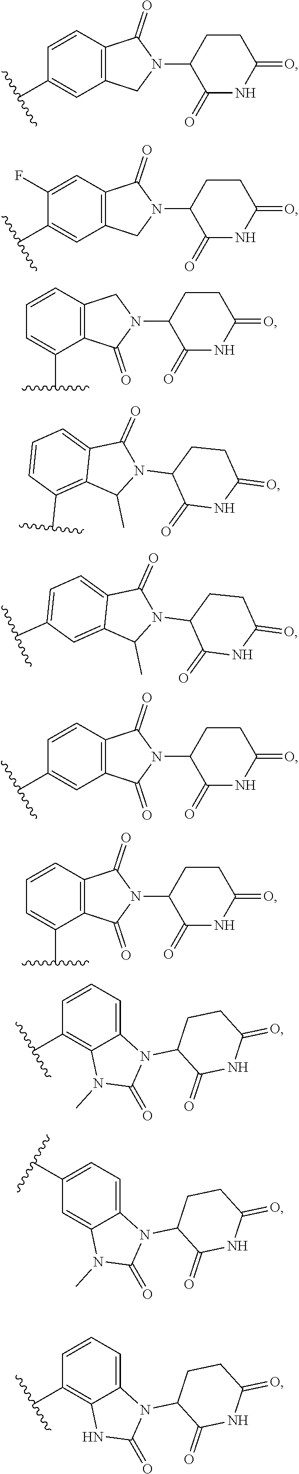

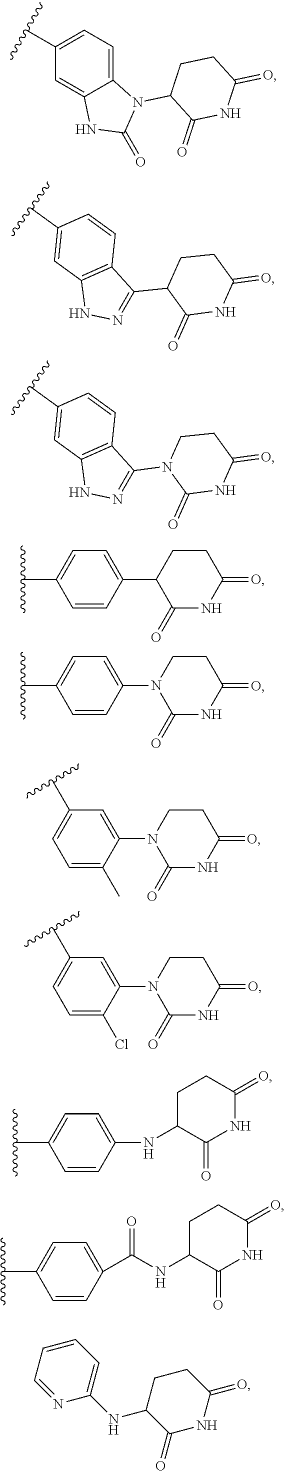

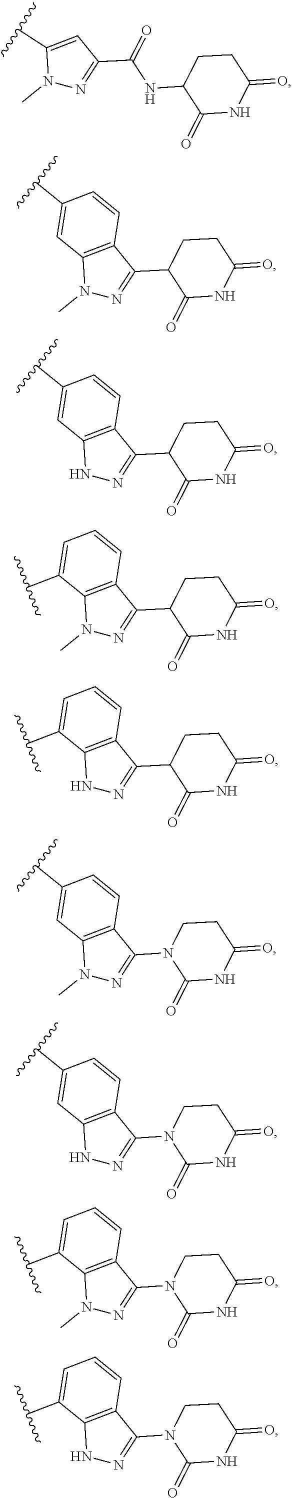

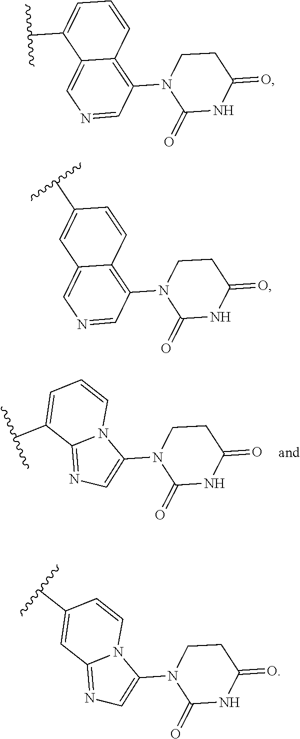 [SMILES: *c1cccc2c(N3CCC(=O)NC3=O)cncc12 ; *c1ccc2c(N3CCC(=O)NC3=O)cncc2c1 ; *c1cccn2c(N3CCC(=O)NC3=O)cnc12 ; "and" ; *c1ccn2c(N3CCC(=O)NC3=O)cnc2c1]

Embodiment 420. The compound of any one of embodiments 1-365, or a pharmaceutically acceptable salt thereof, wherein LBM is selected from the group consisting of

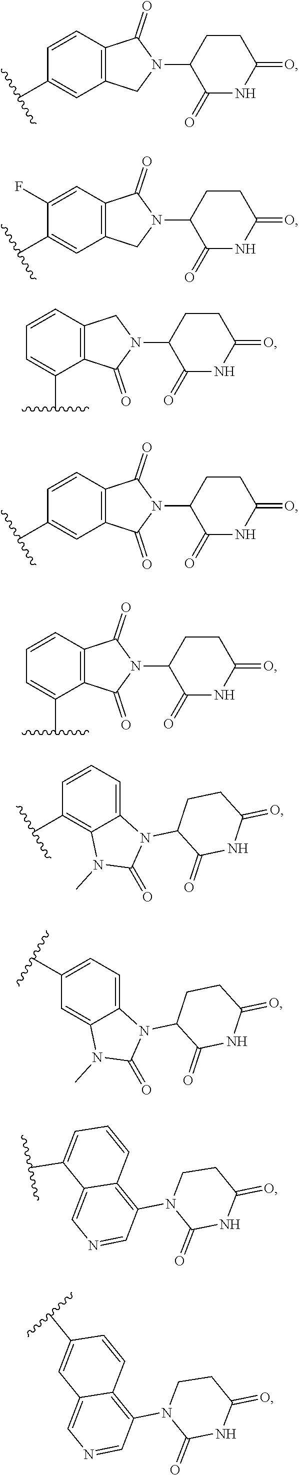

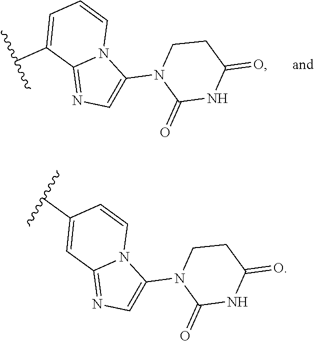 [SMILES: *c1cccn2c(N3CCC(=O)NC3=O)cnc12 ; *c1ccn2c(N3CCC(=O)NC3=O)cnc2c1]

Embodiment 421. The compound of any one of embodiments 1-365 or a pharmaceutically acceptable salt thereof, wherein LBM is selected from

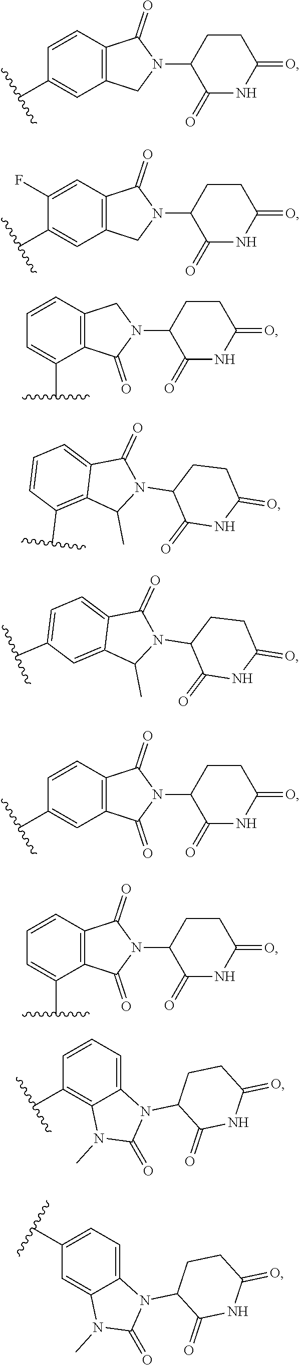

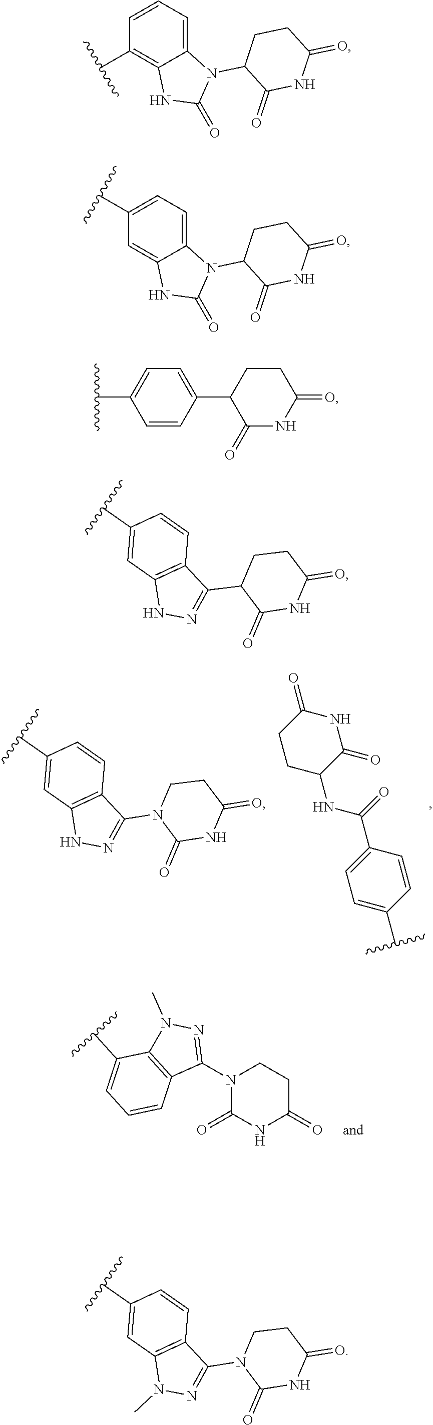

Embodiment 422. The compound of any one of embodiments 1-365, or a pharmaceutically acceptable salt thereof, wherein LBM is selected from

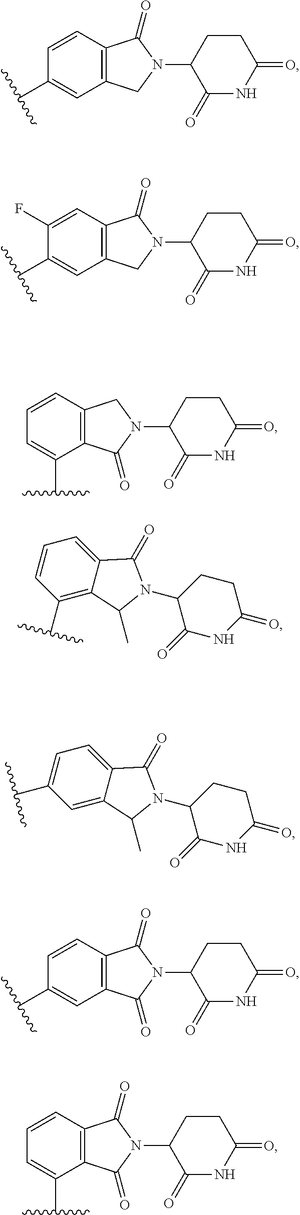

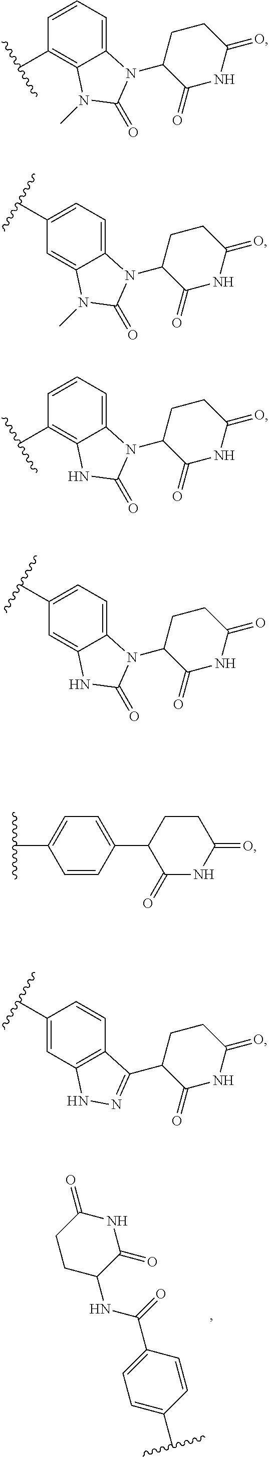

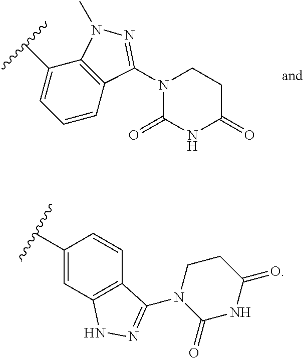 [SMILES: *c1cccc2c(N3CCC(=O)NC3=O)nn(C)c12 ; *c1ccc2c(N3CCC(=O)NC3=O)n[nH]c2c1]

Embodiment 423. The compound of any one of embodiments 1-365, or a pharmaceutically acceptable salt thereof, wherein LBM is selected from

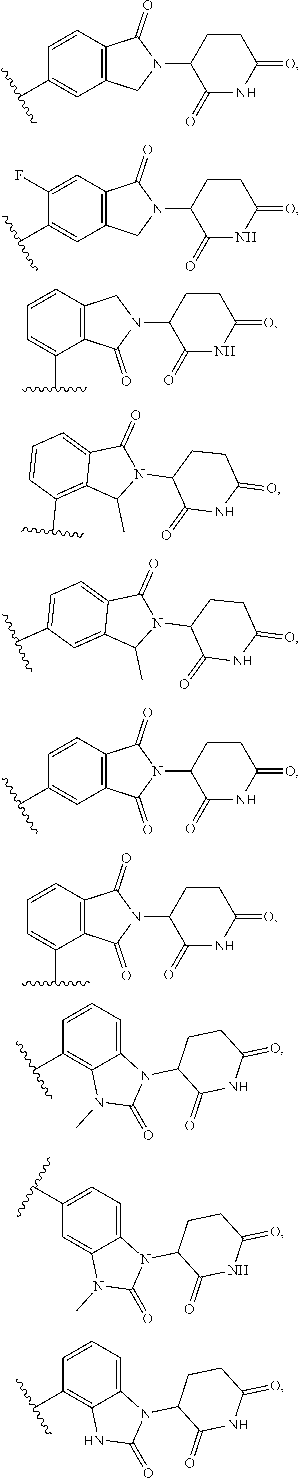

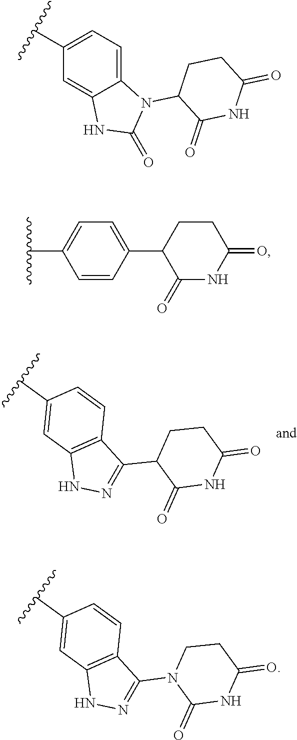 [SMILES: *c1ccc2c(c1)[nH]c(=O)n2C1CCC(=O)NC1=O ; *c1ccc(C2CCC(=O)NC2=O)cc1 ; *c1ccc2c(C3CCC(=O)NC3=O)n[nH]c2c1 ; *c1ccc2c(N3CCC(=O)NC3=O)n[nH]c2c1]

Embodiment 424. The compound of any one of embodiments 1-365, or a pharmaceutically acceptable salt thereof, wherein LBM is selected from

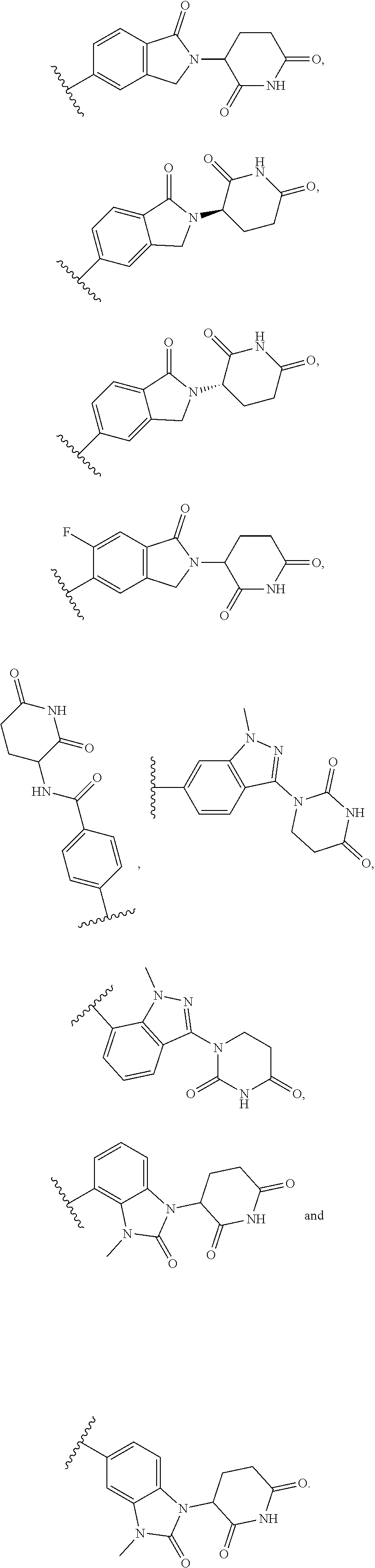

Embodiment 425. The compound of any one of embodiments 1-365, or a pharmaceutically acceptable salt thereof, wherein LBM is selected from

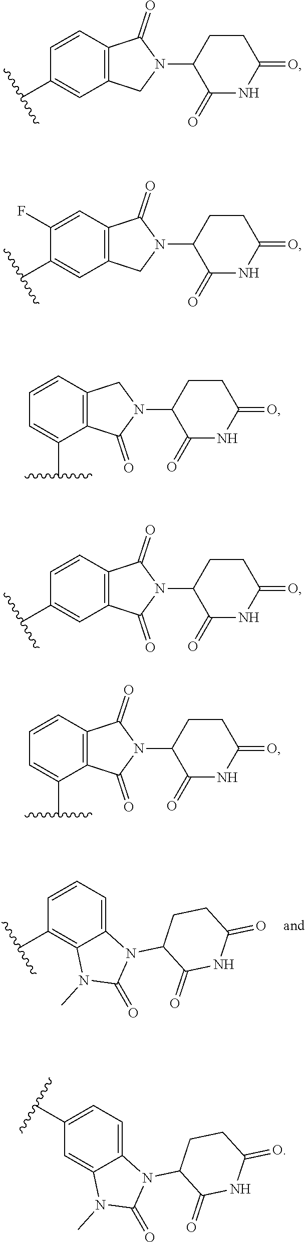

Embodiment 426. The compound of any one of embodiments 1-365 or a pharmaceutically acceptable salt thereof, wherein LBM is selected from

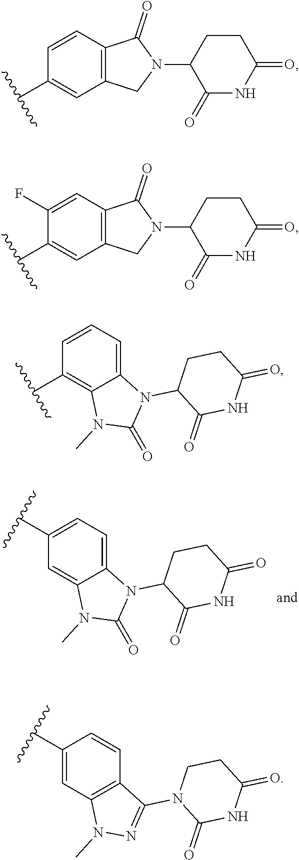 [SMILES: *c1ccc2c(c1)CN(C1CCC(=O)NC1=O)C2=O ; *c1cc2c(cc1F)C(=O)N(C1CCC(=O)NC1=O)C2 ; *c1cccc2c1n(C)c(=O)n2C1CCC(=O)NC1=O ; *c1ccc2c(c1)n(C)c(=O)n2C1CCC(=O)NC1=O ; *c1ccc2c(N3CCC(=O)NC3=O)nn(C)c2c1]

Embodiment 427. The compound of any one of embodiments 1-365, or a pharmaceutically acceptable salt thereof, wherein LBM is

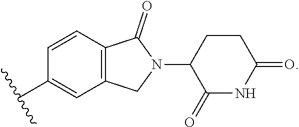 [SMILES: *c1ccc2c(c1)CN(C1CCC(=O)NC1=O)C2=O]

Embodiment 428. The compound of any one of embodiments 1-365, or a pharmaceutically acceptable salt thereof, wherein LBM is

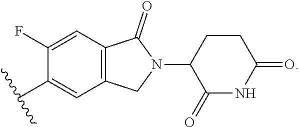 [SMILES: *c1cc2c(cc1F)C(=O)N(C1CCC(=O)NC1=O)C2]

Embodiment 429. The compound of any one of embodiments 1-365, or a pharmaceutically acceptable salt thereof, wherein LBM is

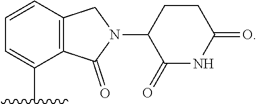 [SMILES: *c1cccc2c1C(=O)N(C1CCC(=O)NC1=O)C2]

Embodiment 430. The compound of any one of embodiments 1-365, or a pharmaceutically acceptable salt thereof, wherein LBM is

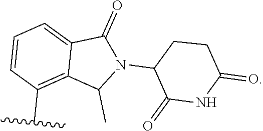 [SMILES: *c1cccc2c1C(C)N(C1CCC(=O)NC1=O)C2=O]

Embodiment 431. The compound of any one of embodiments 1-365, or a pharmaceutically acceptable salt thereof, wherein LBM is

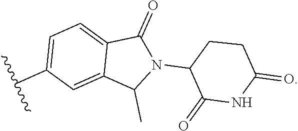 [SMILES: *c1ccc2c(c1)C(C)N(C1CCC(=O)NC1=O)C2=O]

Embodiment 432. The compound of any one of embodiments 1-365, or a pharmaceutically acceptable salt thereof, wherein LBM is

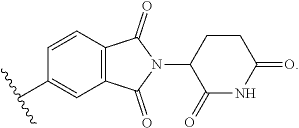 [SMILES: *c1ccc2c(c1)C(=O)N(C1CCC(=O)NC1=O)C2=O]

Embodiment 433. The compound of any one of embodiments 1-365, or a pharmaceutically acceptable salt thereof, wherein LBM is

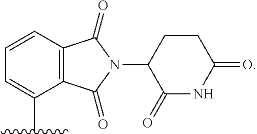 [SMILES: *c1cccc2c1C(=O)N(C1CCC(=O)NC1=O)C2=O]

Embodiment 434. The compound of any one of embodiments 1-365, or a pharmaceutically acceptable salt thereof, wherein LBM is

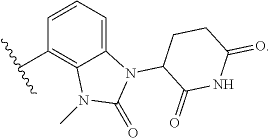 [SMILES: *c1cccc2c1n(C)c(=O)n2C1CCC(=O)NC1=O]

Embodiment 435. The compound of any one of embodiments 1-365, or a pharmaceutically acceptable salt thereof, wherein LBM is

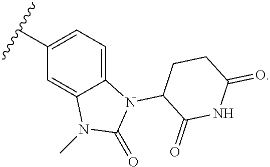 [SMILES: *c1ccc2c(c1)n(C)c(=O)n2C1CCC(=O)NC1=O]

Embodiment 436. The compound of any one of embodiments 1-365, or a pharmaceutically acceptable salt thereof, wherein LBM is

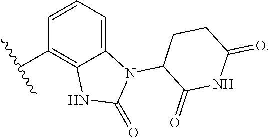 [SMILES: *c1cccc2c1[nH]c(=O)n2C1CCC(=O)NC1=O]

Embodiment 437. The compound of any one of embodiments 1-365, or a pharmaceutically acceptable salt thereof, wherein LBM is

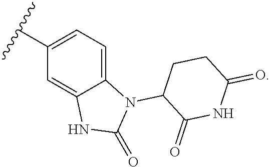 [SMILES: *c1ccc2c(c1)[nH]c(=O)n2C1CCC(=O)NC1=O]

Embodiment 438. The compound of any one of embodiments 1-365, or a pharmaceutically acceptable salt thereof, wherein LBM is

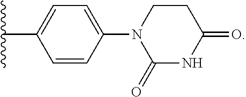 [SMILES: *c1ccc(N2CCC(=O)NC2=O)cc1]

Embodiment 439. The compound of any one of embodiments 1-365, or a pharmaceutically acceptable salt thereof, wherein LBM is

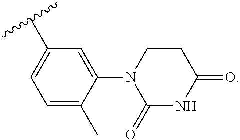 [SMILES: *c1ccc(C)c(N2CCC(=O)NC2=O)c1]

Embodiment 440. The compound of any one of embodiments 1-365, or a pharmaceutically acceptable salt thereof, wherein LBM is

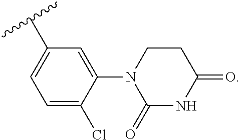 [SMILES: *c1ccc(Cl)c(N2CCC(=O)NC2=O)c1]

Embodiment 441. The compound of any one of embodiments 1-365, or a pharmaceutically acceptable salt thereof, wherein LBM is,

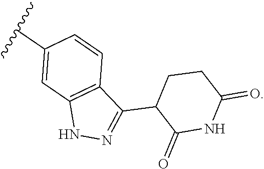 [SMILES: *c1ccc2c(C3CCC(=O)NC3=O)n[nH]c2c1]

Embodiment 442. The compound of any one of embodiments 1-365, or a pharmaceutically acceptable salt thereof, wherein LBM is

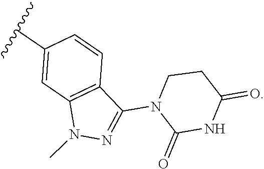 [SMILES: *c1ccc2c(N3CCC(=O)NC3=O)nn(C)c2c1]

Embodiment 443. The compound of any one of embodiments 1-365, or a pharmaceutically acceptable salt thereof, wherein LBM is

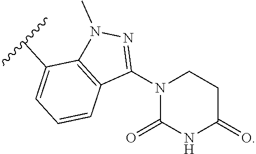 [SMILES: *c1cccc2c(N3CCC(=O)NC3=O)nn(C)c12]

Embodiment 444. The compound of any one of embodiments 1-365, or a pharmaceutically acceptable salt thereof, wherein LBM is

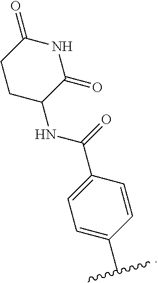 [SMILES: *c1ccc(C(=O)NC2CCC(=O)NC2=O)cc1]

Embodiment 445. The compound of any one of embodiments 1-365, or a pharmaceutically acceptable salt thereof, wherein LBM is

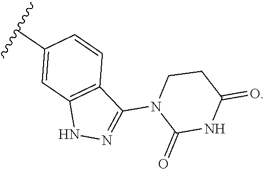 [SMILES: *c1ccc2c(N3CCC(=O)NC3=O)n[nH]c2c1]

Embodiment 446. The compound of any one of embodiments 1-365, or a pharmaceutically acceptable salt thereof, wherein LBM is

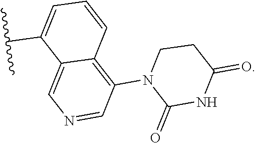 [SMILES: *c1cccc2c(N3CCC(=O)NC3=O)cncc12]

Embodiment 447. The compound of any one of embodiments 1-365, or a pharmaceutically acceptable salt thereof, wherein LBM is

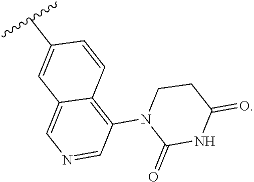 [SMILES: *c1ccc2c(N3CCC(=O)NC3=O)cncc2c1]

Embodiment 448. The compound of any one of embodiments 1-365, or a pharmaceutically acceptable salt thereof, wherein LBM is

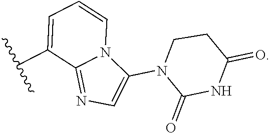 [SMILES: *c1cccn2c(N3CCC(=O)NC3=O)cnc12]

Embodiment 449. The compound of any one of embodiments 1-365, or a pharmaceutically acceptable salt thereof, wherein LBM is

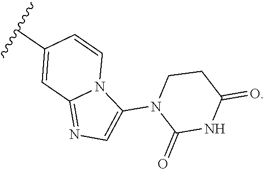 [SMILES: *c1ccn2c(N3CCC(=O)NC3=O)cnc2c1]

Embodiment 450. The compound of any one of embodiments 1-449, or a pharmaceutically acceptable salt thereof, wherein the compound is selected from the compounds of Table 1.

Embodiment 451. A pharmaceutical composition comprising a compound of any one of embodiments 1-450, or a pharmaceutically acceptable salt thereof, and a pharmaceutically acceptable carrier, adjuvant, or diluent.

Embodiment 452. A method of inhibiting CDK2 and/or CCNE (CCNE1 and/or CCNE2) signaling in a sample, e.g., in vivo or in vitro, by contacting CDK2 and/or CCNE (CCNE1 and/or CCNE2) with a compound of any one of embodiments 1-450, or a pharmaceutically acceptable salt thereof, or a composition of embodiment 451.

Embodiment 453. Use of a compound of any one of embodiments 1-450 or a composition of embodiment 451 in a method of inhibiting CDK2 and/or CCNE (CCNE1 and/or CCNE2) signaling in a sample, e.g., in vivo or in vitro, by contacting CDK2 and/or CCNE (CCNE1 and/or CCNE2) with the compound or composition.

Embodiment 454. Use of a compound of any one of embodiments 1-450 or a composition of embodiment 451 in the manufacturing of a medicament for inhibiting CDK2 and/or CCNE (CCNE1 and/or CCNE2) signaling in a sample, e.g., in vivo or in vitro.

Embodiment 455. A compound of any one of embodiments 1-450 or a composition of embodiment 451 for use in a method of inhibiting CDK2 and/or CCNE (CCNE1 and/or CCNE2) signaling in a sample, e.g., in vivo or in vitro, by contacting CDK2 and/or CCNE (CCNE1 and/or CCNE2) with the compound or composition.

Embodiment 456. A compound of any one of embodiments 1-450 or a composition of embodiment 451 for use in manufacturing of a medicament for inhibiting CDK2 and/or CCNE (CCNE1 and/or CCNE2) signaling in a sample, e.g., in vivo or in vitro, by contacting CDK2 and/or CCNE (CCNE1 and/or CCNE2) with the compound or composition.

Embodiment 457. The method, use, compound or composition for use of any one of embodiments 452-456, wherein the inhibiting of CDK2 and/or CCNE (CCNE1 and/or CCNE2) signaling comprises reducing the signaling activity of CDK2 and/or CCNE (CCNE1 and/or CCNE2) by at least 1%, 2%, 5%, 7.5%, 10%, 15%, 20%, 25%, 30%, 35%, 40%, 45%, 50%, 55%, 60%, 65%, 70%, 75%, 80%, 85%, 90%, 95%, 99%, e.g., relative to a reference standard.

Embodiment 458. The method, use, compound or composition for use of any one of embodiments 452-456, wherein the inhibiting of CDK2 and/or CCNE (CCNE1 and/or CCNE2) signaling comprises reducing the signaling activity of CDK2 and/or CCNE (CCNE1 and/or CCNE2) by at least 1-fold, 1.5-fold, 2-fold, 3-fold, 5-fold, 10-fold, 20-fold, 30-fold, 50-fold, 100-fold, or more, e.g., relative to a reference standard.

Embodiment 459. A method of inhibiting CDK2 signaling in a sample, e.g., in vivo or in vitro, by contacting CDK2 with a compound of any one of embodiments 1-450, or a pharmaceutically acceptable salt thereof, or a composition of embodiment 451.

Embodiment 460. Use of a compound of any one of embodiments 1-450, or a pharmaceutically acceptable salt thereof, or a composition of embodiment 451 in a method of inhibiting CDK2 signaling in a sample, e.g., in vivo or in vitro, by contacting CDK2 with a compound of any one of embodiments 1-450, or a pharmaceutically acceptable salt thereof, or a composition of embodiment 451.

Embodiment 461. Use of a compound of any one of embodiments 1-450, or a pharmaceutically acceptable salt thereof, or a composition of embodiment 451 in the manufacturing of a medicament for inhibiting CDK2 signaling in a sample, e.g., in vivo or in vitro.

Embodiment 462. A compound of any one of embodiments 1-450, or a pharmaceutically acceptable salt thereof, or a composition of embodiment 451 for use in a method of inhibiting CDK2 signaling in a sample, e.g., in vivo or in vitro, by contacting CDK2 with a compound of any one of embodiments 1-450, or a pharmaceutically acceptable salt thereof, or a composition of embodiment 451.

Embodiment 463. A compound of any one of embodiments 1-450, or a pharmaceutically acceptable salt thereof, or a composition of embodiment 451 for use in the manufacturing of a medicament for inhibiting CDK2 signaling in a sample, e.g., in vivo or in vitro.

Embodiment 464. The method, use, compound or composition for use of any one of embodiments 459-463, wherein the inhibiting of CDK2 signaling comprises reducing the signaling activity of CDK2 by at least 1%, 2%, 5%, 7.5%, 10%, 15%, 20%, 25%, 30%, 35%, 40%, 45%, 50%, 55%, 60%, 65%, 70%, 75%, 80%, 85%, 90%, 95%, 99%, e.g., relative to a reference standard.

Embodiment 465. The method, use, compound or composition for use of any one of embodiments 459-463, wherein the inhibiting of CDK2 signaling comprises reducing the signaling activity of CDK2 by at least 1-fold, 1.5-fold, 2-fold, 3-fold, 5-fold, 10-fold, 20-fold, 30-fold, 50-fold, 100-fold, or more, e.g., relative to a reference standard.

Embodiment 466. A method of inhibiting CCNE (CCNE1 and/or CCNE2) signaling in a sample, e.g., in vivo or in vitro, by contacting CCNE (CCNE1 and/or CCNE2) with a compound of any one of embodiments 1-450, or a pharmaceutically acceptable salt thereof, or a composition of embodiment 451.

Embodiment 467. Use of a compound of any one of embodiments 1-450, or a pharmaceutically acceptable salt thereof, or a composition of embodiment 451 in a method of inhibiting CCNE (CCNE1 and/or CCNE2) signaling in a sample, e.g., in vivo or in vitro, by contacting CCNE (CCNE1 and/or CCNE2) with a compound of any one of embodiments 1-450, or a pharmaceutically acceptable salt thereof, or a composition of embodiment 451.

Embodiment 468. Use of a compound of any one of embodiments 1-450, or a pharmaceutically acceptable salt thereof, or a composition of embodiment 451 in the manufacturing of a medicament for inhibiting CCNE (CCNE1 and/or CCNE2) signaling in a sample, e.g., in vivo or in vitro.

Embodiment 469. A compound of any one of embodiments 1-450, or a pharmaceutically acceptable salt thereof, or a composition of embodiment 451 for use in a method of inhibiting CCNE (CCNE1 and/or CCNE2) signaling in a sample, e.g., in vivo or in vitro, by contacting CCNE (CCNE1 and/or CCNE2) with a compound of any one of embodiments 1-450, or a pharmaceutically acceptable salt thereof, or a composition of embodiment 451.

Embodiment 470. A compound of any one of embodiments 1-450, or a pharmaceutically acceptable salt thereof, or a composition of embodiment 451 for use in the manufacturing of a medicament for inhibiting CCNE (CCNE1 and/or CCNE2) signaling in a sample, e.g., in vivo or in vitro.

Embodiment 471. The method, use, compound or composition for use of any one of embodiments 466-470, wherein the inhibiting of CCNE (CCNE1 and/or CCNE2) signaling comprises reducing the signaling activity of CCNE (CCNE1 and/or CCNE2) by at least 1%, 2%, 5%, 7.5%, 10%, 15%, 20%, 25%, 30%, 35%, 40%, 45%, 50%, 55%, 60%, 65%, 70%, 75%, 80%, 85%, 90%, 95%, 99%, e.g., relative to a reference standard.

Embodiment 472. The method, use, compound or composition for use of any one of embodiments 466-470, wherein the inhibiting CCNE (CCNE1 and/or CCNE2) signaling comprises reducing the signaling activity of CCNE (CCNE1 and/or CCNE2) by at least 1-fold, 1.5-fold, 2-fold, 3-fold, 5-fold, 10-fold, 20-fold, 30-fold, 50-fold, 100-fold, or more, e.g., relative to a reference standard.

Embodiment 473. A method of inhibiting CDK2 and CCNE (CCNE1 and/or CCNE2) signaling in a sample, e.g., in vivo or in vitro, by contacting CDK2 and/or CCNE (CCNE1 and/or CCNE2) with a compound of any one of embodiments 1-450, or a pharmaceutically acceptable salt thereof, or a composition of embodiment 451.

Embodiment 474. Use of a compound of any one of embodiments 1-450, or a pharmaceutically acceptable salt thereof, or a composition of embodiment 451 in a method of inhibiting CDK2 and CCNE (CCNE1 and/or CCNE2) signaling in a sample, e.g., in vivo or in vitro, by contacting CDK2 and/or CCNE (CCNE1 and/or CCNE2) with a compound of any one of embodiments 1-450, or a pharmaceutically acceptable salt thereof, or a composition of embodiment 451.

Embodiment 475. Use of a compound of any one of embodiments 1-450, or a pharmaceutically acceptable salt thereof, or a composition of embodiment 451 in the manufacturing of a medicament for inhibiting CDK2 and CCNE (CCNE1 and/or CCNE2) signaling in a sample, e.g., in vivo or in vitro.

Embodiment 476. A compound of any one of embodiments 1-450, or a pharmaceutically acceptable salt thereof, or a composition of embodiment 451 for use in a method of inhibiting CDK2 and CCNE (CCNE1 and/or CCNE2) signaling in a sample, e.g., in vivo or in vitro, by contacting CDK2 and/or CCNE (CCNE1 and/or CCNE2) with a compound of any one of embodiments 1-450, or a pharmaceutically acceptable salt thereof, or a composition of embodiment 451.

Embodiment 477. A compound of any one of embodiments 1-450, or a pharmaceutically acceptable salt thereof, or a composition of embodiment 451 for use in the manufacturing of a medicament for inhibiting CDK2 and CCNE (CCNE1 and/or CCNE2) signaling in a sample, e.g., in vivo or in vitro.

Embodiment 478. The method, use, compound or composition for use of any one of embodiments 473-477, wherein the inhibiting of CDK2 and CCNE (CCNE1 and/or CCNE2) signaling comprises reducing the signaling activity of CDK2 and/or CCNE (CCNE1 and/or CCNE2) by at least 1%, 2%, 5%, 7.5%, 10%, 15%, 20%, 25%, 30%, 35%, 40%, 45%, 50%, 55%, 60%, 65%, 70%, 75%, 80%, 85%, 90%, 95%, 99%, e.g., relative to a reference standard.

Embodiment 479. The method, use, compound or composition for use of any one of embodiments 473-477, wherein the inhibiting of CDK2 and CCNE (CCNE1 and/or CCNE2) signaling comprises reducing the signaling activity of CDK2 and/or CCNE (CCNE1 and/or CCNE2) by at least 1-fold, 1.5-fold, 2-fold, 3-fold, 5-fold, 10-fold, 20-fold, 30-fold, 50-fold, 100-fold, or more, e.g., relative to a reference standard.

Embodiment 480. A method of treating a CDK2 and/or CCNE (CCNE1 and/or CCNE2)-mediated disorder in a patient in need thereof, comprising administering to the patient a compound of any one of any one of embodiments 1-450, or a pharmaceutically acceptable salt thereof, or a composition of embodiment 451.

Embodiment 481. Use of a compound of any one of any one of embodiments 1-450, or a pharmaceutically acceptable salt thereof, or a composition of embodiment 451 in a method of treating a CDK2 and/or CCNE (CCNE1 and/or CCNE2)-mediated disorder in a patient in need thereof, comprising administering to the patient a compound of any one of any one of embodiments 1-450, or a pharmaceutically acceptable salt thereof, or a composition of embodiment 451.

Embodiment 482. Use of a compound of any one of any one of embodiments 1-450, or a pharmaceutically acceptable salt thereof, or a composition of embodiment 451 in the manufacturing of a medicament for treating a CDK2 and/or CCNE (CCNE1 and/or CCNE2)-mediated disorder in a patient in need thereof.

Embodiment 483. A compound of any one of any one of embodiments 1-450, or a pharmaceutically acceptable salt thereof, or a composition of embodiment 451 for use in a method of treating a CDK2 and/or CCNE (CCNE1 and/or CCNE2)-mediated disorder in a patient in need thereof, comprising administering to the patient a compound of any one of any one of embodiments 1-450, or a pharmaceutically acceptable salt thereof, or a composition of embodiment 451.

Embodiment 484. A compound of any one of any one of embodiments 1-450, or a pharmaceutically acceptable salt thereof, or a composition of embodiment 451 for use in the manufacturing of a medicament for treating a CDK2 and/or CCNE (CCNE1 and/or CCNE2)-mediated disorder in a patient in need thereof.

Embodiment 485. The method, use, compound or composition for use of any one of embodiments 480-484, wherein the CDK2 and/or CCNE (CCNE1 and/or CCNE2)-mediated disorder is cancer.

Embodiment 486. A method of treating a CDK2-mediated disorder in a patient in need thereof, comprising administering to the patient a compound of any one of any one of embodiments 480-484, or a pharmaceutically acceptable salt thereof, or a composition of embodiment 451.

Embodiment 487. Use of a compound of any one of any one of embodiments 1-450, or a pharmaceutically acceptable salt thereof, or a composition of embodiment 451 in a method of treating a CDK2-mediated disorder in a patient in need thereof, comprising administering to the patient a compound of any one of any one of embodiments 1-450, or a pharmaceutically acceptable salt thereof, or a composition of embodiment 451.

Embodiment 488. Use of a compound of any one of any one of embodiments 1-450, or a pharmaceutically acceptable salt thereof, or a composition of embodiment 451 in the manufacturing of a medicament for treating a CDK2-mediated disorder in a patient in need thereof.

Embodiment 489. A compound of any one of any one of embodiments 1-450, or a pharmaceutically acceptable salt thereof, or a composition of embodiment 451 for use in a method of treating a CDK2-mediated disorder in a patient in need thereof, comprising administering to the patient a compound of any one of any one of embodiments 1-450, or a pharmaceutically acceptable salt thereof, or a composition of embodiment 451.

Embodiment 490. A compound of any one of any one of embodiments 1-450, or a pharmaceutically acceptable salt thereof, or a composition of embodiment 451 for use in the manufacturing of a medicament for treating a CDK2-mediated disorder in a patient in need thereof.

Embodiment 491. The method, use, compound or composition for use of any one of embodiments 486-490, wherein the CDK2-mediated disorder is cancer.

Embodiment 492. A method of treating a CCNE (CCNE1 and/or CCNE2)-mediated disorder in a patient in need thereof, comprising administering to the patient a compound of any one of any one of embodiments 1-450, or a pharmaceutically acceptable salt thereof, or a composition of embodiment 451.

Embodiment 493. Use of a compound of any one of any one of embodiments 1-450, or a pharmaceutically acceptable salt thereof, or a composition of embodiment 451 in a method of treating a CCNE (CCNE1 and/or CCNE2)-mediated disorder in a patient in need thereof, comprising administering to the patient a compound of any one of any one of embodiments 1-450, or a pharmaceutically acceptable salt thereof, or a composition of embodiment 451.

Embodiment 494. Use of a compound of any one of any one of embodiments 1-450, or a pharmaceutically acceptable salt thereof, or a composition of embodiment 451 in the manufacturing of a medicament for treating a CCNE (CCNE1 and/or CCNE2)-mediated disorder in a patient in need thereof.

Embodiment 495. A compound of any one of any one of embodiments 1-450, or a pharmaceutically acceptable salt thereof, or a composition of embodiment 451 for use in a method of treating a CCNE (CCNE1 and/or CCNE2)-mediated disorder in a patient in need thereof, comprising administering to the patient a compound of any one of any one of embodiments 1-450, or a pharmaceutically acceptable salt thereof, or a composition of embodiment 451.

Embodiment 496. A compound of any one of any one of embodiments 1-450, or a pharmaceutically acceptable salt thereof, or a composition of embodiment 451 for use in the manufacturing of a medicament for treating a CCNE (CCNE1 and/or CCNE2)-mediated disorder in a patient in need thereof.

Embodiment 497. The method, use, compound or composition for use of any one of embodiments 492-496, wherein the CCNE (CCNE1 and/or CCNE2)-mediated disorder is cancer.

Embodiment 498. A method of treating a CDK2 and CCNE (CCNE1 and/or CCNE2)-mediated disorder in a patient in need thereof, comprising administering to the patient a compound of any one of any one of embodiments 1-450, or a pharmaceutically acceptable salt thereof, or a composition of embodiment 451.

Embodiment 499. Use of a compound of any one of any one of embodiments 1-450, or a pharmaceutically acceptable salt thereof, or a composition of embodiment 451 in a method of treating a CDK2 and CCNE (CCNE1 and/or CCNE2)-mediated disorder in a patient in need thereof, comprising administering to the patient a compound of any one of any one of embodiments 1-450, or a pharmaceutically acceptable salt thereof, or a composition of embodiment 451.

Embodiment 500. Use of a compound of any one of any one of embodiments 1-450, or a pharmaceutically acceptable salt thereof, or a composition of embodiment 451 in the manufacturing of a medicament for treating a CDK2 and CCNE (CCNE1 and/or CCNE2)-mediated disorder in a patient in need thereof.

Embodiment 501. A compound of any one of any one of embodiments 1-450, or a pharmaceutically acceptable salt thereof, or a composition of embodiment 451 for use in a method of treating a CDK2 and CCNE (CCNE1 and/or CCNE2)-mediated disorder in a patient in need thereof, comprising administering to the patient a compound of any one of any one of embodiments 1-450, or a pharmaceutically acceptable salt thereof, or a composition of embodiment 451.

Embodiment 502. A compound of any one of any one of embodiments 1-450, or a pharmaceutically acceptable salt thereof, or a composition of embodiment 451 for use in the manufacturing of a medicament for treating a CDK2 and CCNE (CCNE1 and/or CCNE2)-mediated disorder in a patient in need thereof.

Embodiment 503. The method, use, compound or composition for use of any one of embodiments 498-502, wherein the CDK2 and CCNE (CCNE1 and/or CCNE2)-mediated disorder is cancer.

Embodiment 504. The method, use, compound or composition for use of any one of embodiments 1, 491, 497 and 503, wherein the cancer is selected from ovarian cancer, gastric cancer, uterine cancer (e.g., endometrial cancer), and breast cancer (e.g., triple negative breast cancer (TNBC), hormone-receptor positive (HR+) breast cancer, HER2 positive (HER2+) positive breast cancer).

Embodiment 505. The method, use, compound or composition for use of any one of embodiments 1, 491, 497, 503 and 504, wherein the cancer is resistant to treatment with CDK 4/6 inhibitors.

### EXAMPLES

In order that the invention(s) described herein may be more fully understood, the following examples are set forth. The synthetic and biological examples described in this application are offered to illustrate the compounds, pharmaceutical compositions, and methods provided herein and are not to be construed in any way as limiting their scope. In the synthetic examples below, the descriptions of experimental procedures within a reaction sequence are listed in numerical order.

### Abbreviations

### General

anhy. anhydrous
aq. aqueous
satd. saturated
mm(s) minute(s)
hr(s) hour(s)
mL milliliter
mmol millimole(s)
mol mole(s)
MS mass spectrometry
NMR nuclear magnetic resonance
TLC thin layer chromatography
HPLC high-performance liquid chromatography
Me methyl
i-Pr iso-propyl
t-Bu tert-butyl
Ph phenyl
Et ethyl
Bz benzoyl

### Spectrum

Hzhertz
δ chemical shift
J coupling constant
s singlet
d doublet
t triplet
q quartet
m multiplet
br broad
qd quartet of doublets
dquin doublet of quintets
dd doublet of doublets
dt doublet of triplets

### Solvents and Reagents

(i-PrO)₄Ti titanium tetraisopropoxide
9-BBN 9-borabicyclo[3.3.1]nonane
AcCl acetyl chloride
ACN Acetonitrile
AcOH acetic acid
ADDP 1,1′-(azodicarbonyl)dipiperidine
AlaOH alanine
BHT 2,6-di-t-butyl-4-methylphenoxide
BINAP 2,2′-bis(diphenylphosphanyl)-1,1′-binaphthyl
Boc t-butoxycarbonyl
BSA Bovine Serum Albumin
Bu butyl
BzCl benzoyl chloride
CHCl₃ chloroform
CsF cesium fluoride
DAST Diethylaminosulfurtrifluoride
DCC dicyclohexylcarbodiimide
DCM dichloromethane
DIAD diisopropyl azodicarboxylate
DIPEA N,N-diisopropylethylamine
DMAP 4-(dimethylamino)pyridine
DMF dimethylformamide
DMP Dess-Martin periodinane
DMSO dimethyl sulfoxide
dppf 1,1′-bis(diphenylphosphino)ferrocene
DTT DL-Dithiothreitol
Et₂O diethyl ether
Et₃N triethylamine
EtMgBr ethylmagnesium bromide
EtOAc ethyl acetate
EtOH ethyl alcohol
H₂SO₄ sulfuric acid
HCl hydrochloric acid
i-PrMgCl Isopropylmagnesium chloride
K₂CO₃ potassium carbonate
KOH potassium hydroxide
LAH Lithium Aluminium Hydride
LDA lithium diisopropylamide
LDH Lactate Dehydrogenase
LiHMDS lithium hexamethyldisilylamide
LiOH·H₂O lithium hydroxide hydrates
MAD methyl aluminum bis(2,6-di-t-butyl-4-methylphenoxide)
MeCN acetonitrile
MeOH methyl alcohol
MTBE methyl tert-butyl ether
Na₂CO₃ sodium carbonate
Na₂S₂O₃ sodium thiosulfate
Na₂SO₄ sodium sulfate
NaBH₄ sodium borohydride
NaBH₄ sodium borohydride
NADH β-Nicotinamide adenine dinucleotide, reduced
NaHCO₃ sodium bicarbonate
NaOH sodium hydroxide
NBS N-bromosuccinimide
NH₄Cl ammonium chloride
PCC pyridinium chlorochromate
Pd(t-Bu₃P)₂ bis(tri-tert-butylphosphine)palladium(0)
PE petroleum ether
PEP Phospho(enol)pyruvic acid
Py pyridine
RuPhos 2-dicyclohexylphosphino-2′,6′-diisopropoxybiphenyl
TBAF tetra-n-butylammonium fluoride
TBS t-butyldimethylsilyl
TBSCl tert-Butyl(chloro)dimethylsilane
t-BuOK potassium tert-butoxide
^{t}BuXPhos 2-di-tert-butylphosphino-2′,4′,6′-triisopropylbiphenyl
TEA triethylamine
TFA trifluoroacetic acid
THF tetrahydrofuran
Ti(O′Pr)₄ tetraisopropoxytitanium
TMS trimethylsilyl
TMSCF₃ (Trifluoromethyl)trimethylsilane
Ts p-toluenesulfonyl
Xphos Dicyclohexyl[2′,4′,6′-tris(propan-2-yl)[1,1′-biphenyl]-2-yl]phosphane

### Chemistry Examples

### Intermediate 1,4-{2-Bromo-8-isopropoxy-[1,2,41triazolo[1,5-c]pyrimidin-7-yl}-1-(1-ethoxyethyl)pyrazole (CI-1)

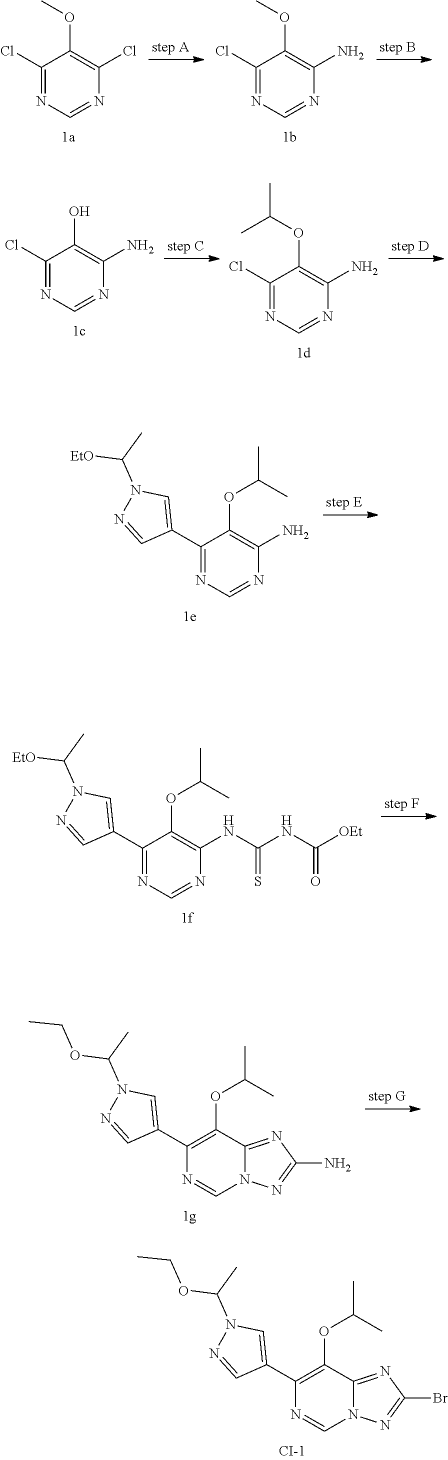

### Step A. 6-Chloro-5-methoxypyrimidin-4-amine (1b)

A mixture of 1a (25 g, 139.665 mmol, 1 equiv) and ammonium hydroxide (112.5 mL, 3210.067 mmol, 45.97 equiv) in n-BuOH (250 mL) was stirred for 16 h at 85° C. in autoclave. The reaction was monitored by LCMS. The mixture was allowed to cool down to room temperature. The resulting mixture was concentrated under reduced pressure. The crude product was re-crystallized from brine to afford 1b (15 g, 67.31%) as a white solid. LCMS (ESI, m/z): [M+H]⁺ 160.0

### Step B. 4-Amino-6-chloropyrimidin-5-ol (1c)

A mixture of 1b (20 g, 125.337 mmol, 1 equiv) and BBr₃ (120.03 mL, 1269.664 mmol, 10.13 equiv) in dichloromethane (300 mL) was stirred for 4 days at room temperature under nitrogen atmosphere. The reaction was monitored by LCMS. The resulting mixture was concentrated under reduced pressure. The reaction was quenched by the addition of isopropyl alcohol (25 mL) at 0° C. The crude product was re-crystallized from ethyl ether to afford 1c (15 g, 82.22%) as a white solid. LCMS (ESI, m/z): [M+H]⁺ 146.0

### Step C. 6-Chloro-5-isopropoxypyrimidin-4-amine (1d)

A mixture of 1c (20 g, 137.410 mmol, 1 equiv), 2-iodopropane (39.01 g, 229.480 mmol, 1.67 equiv) and potassium carbonate (18.99 g, 137.405 mmol, 1 equiv) in N,N-dimethylformamide (150 mL) was stirred for 16 h at 60° C. under nitrogen atmosphere. The reaction was monitored by LCMS. The mixture was allowed to cool down to room temperature. The resulting mixture was extracted with ethyl acetate (4×400 mL). The combined organic layers were washed with brine (10×100 mL), dried over anhydrous sodium sulfate. After filtration, the filtrate was concentrated under reduced pressure. The residue was purified by silica gel column chromatography, eluted with petroleum ether/ethyl acetate (1:1) to afford 1d (12 g, 46.60%) as a white solid. LCMS (ESI, m/z): [M+H]⁺ 188.0

### Step D. 6-[l-(1-Eethoxyethyl)pyrazol-4-yl]-5-isopropoxypyrimidin-4-amine) (1e)

A mixture of 1d (20 g, 106.593 mmol, 1 equiv),1-(1-ethoxyethyl)-4-(4,4,5,5-tetramethyl-1,3,2-dioxaborolan-2-yl)pyrazole (70.92 g, 266.483 mmol, 2.50 equiv), Pd(dppf)C₁₋₂.dichloromethane (4.34 g, 5.330 mmol, 0.05 equiv), and potassium phosphate tribasic (67.88 g, 319.779 mmol, 3 equiv) in 1,4-dioxane(220 mL) was stirred for 2 h at 90° C. under nitrogen atmosphere. The reaction was monitored by LCMS. The mixture was allowed to cool down to room temperature. The resulting mixture was concentrated under reduced pressure. The residue was purified by silica gel column chromatography, eluted with petroleum ether/ethyl acetate (1:1) to afford 1e (20 g, 64.40%) as a brown solid. LCMS (ESI, m/z): [M+H]⁺ 292.1

### Step E. Ethyl N—({6-[1-(1-ethoxyethyl)pyrazol-4-yl]-5-isopropoxypyrimidin-4-yl}carbamothioyl) carbamate (If)

A mixture of 1e (9 g, 30.890 mmol, 1 equiv) and ethyl N-carbothioylcarbamate (4.86 g, 37.057 mmol, 1.20 equiv) in acetonitrile (90 mL) was stirred for 2 h at 90° C. under nitrogen atmosphere. The reaction was monitored by LCMS. The mixture was allowed to cool down to room temperature. The resulting mixture was concentrated under reduced pressure. This resulted in if as a brown oil. The crude product was used in the next step directly without further purification. LCMS (ESI, m/z): [M+H]⁺ 423.2

### Step F. 7-[1-(1-Ethoxyethyl)pyrazol-4-yl]-8-isopropoxy-[1,2,4]triazolo[1,5-c]pyrimidin-2-amine (1g)

A mixture of if (11.3 g, 26.746 mmol, 1 equiv), triethylethylenediamine (10.37 g, 80.238 mmol, 3 equiv) and hydroxylamine hydrochloride (5.58 g, 80.238 mmol, 3 equiv) in ethanol (110 mL) was stirred for 2 h at 80° C. under nitrogen atmosphere. The reaction was monitored by LCMS. The mixture was allowed to cool down to room temperature. The resulting mixture was diluted with dichloromethane (200 mL). The combined organic layers were washed with brine (3×30 mL), dried over anhydrous sodium sulfate. After filtration, the filtrate was concentrated under reduced pressure. The residue was purified by silica gel column chromatography, eluted with petroleum ether/ethyl acetate (1:1) to afford if (4 g, 45.13%) as a white solid. LCMS (ESI, m/z): [M+H]⁺ 332.2

### Step F. 4-{2-Bromo-8-isopropoxy-[1,2,4]triazolo[1,5-c]pyrimidin-7-yl}-1-(1-ethoxyethyl)pyrazole (CI-1)

A solution of 1g (11 g, 33.195 mmol, 1 equiv), CuBr (5.24 g, 36.515 mmol, 1.1 equiv) and ^{t}BuONO (6.85 g, 66.390 mmol, 2 equiv) in acetonitrile (110 mL) was stirred for 16 h at room temperature under nitrogen atmosphere. Desired product could be detected by LCMS. The resulting mixture was diluted with water (100 mL) and extracted with dichloromethane (2×200 mL). The combined organic layers were washed with brine (3×20 mL), dried over anhydrous sodium sulfate. After filtration, the filtrate was concentrated under reduced pressure. The residue was purified by silica gel column chromatography, eluted with petroleum ether/ethyl acetate (4:1) to afford CI-1 (6 g, 45.73%) as a white solid. LCMS (ESI, m/z): [M+H]⁺ 395.1

### Intermediate 2. N-{7-[1-(1-Ethoxyethyl)pyrazol-4-yl}-8-isopropoxy-[1,2,41triazolo[1,5-c]pyrimidin-2-yl}piperidin-4-amine (CI-2)

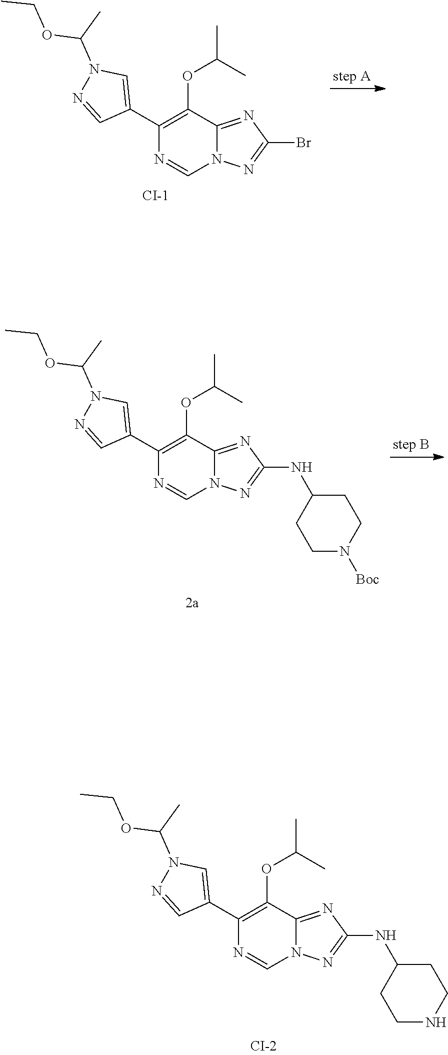 [SMILES: CCOC(C)n1cc(-c2ncn3nc(Br)nc3c2OC(C)C)cn1 ; CCOC(C)n1cc(-c2ncn3nc(NC4CCN(C(=O)OC(C)(C)C)CC4)nc3c2OC(C)C)cn1 ; CCOC(C)n1cc(-c2ncn3nc(NC4CCNCC4)nc3c2OC(C)C)cn1]

### Step A. Tert-butyl 4-({7-[1-(1-ethoxyethyl)pyrazol-4-yl]-8-isopropoxy-[1,2,4]triazolo[1,5-c]pyrimidin-2-yl}amino)piperidine-1-carboxylate (2a)

To a stirred solution of CI-1 (400 mg, 1.012 mmol, 1 equiv) and tert-butyl 4-aminopiperidine-1-carboxylate (243.22 mg, 1.214 mmol, 1.2 equiv) in 1,4-dioxane (4 mL) were added Pd₂(dba)₃ (185.34 mg, 0.202 mmol, 0.2 equiv) and sodium tert-butoxide (291.77 mg, 3.036 mmol, 3 equiv) at room temperature under nitrogen atmosphere. The resulting mixture was stirred for 1 h at 100° C. under nitrogen atmosphere. The reaction was monitored by LCMS. The resulting mixture was concentrated under reduced pressure. The residue was purified by silica gel column chromatography, eluted with petroleum ether/ethyl acetate (1:1) to afford 2a (263 mg, 50.50%) as a yellow solid. LCMS (ESI, m/z): [M+H]⁺ 515.3.

### Step B. N-{7-[1-(1-Ethoxyethyl)pyrazol-4-yl]-8-isopropoxy-[1,2,4]triazolo[1,5-c]pyrimidin-2-yl}piperidin-4-amine (CI-2)

To a stirred solution of 2a (400 mg, 0.777 mmol, 1 equiv) and trimethylsilyl trifluoromethanesulfonate (518.24 mg, 2.331 mmol, 3 equiv) in dichloromethane (4 mL) was added triethylamine (471.92 mg, 4.662 mmol, 6 equiv) at 0° C. The resulting mixture was stirred for 1 h at room temperature. The reaction was monitored by LCMS. The resulting mixture was diluted with dichloromethane (50 mL) and was washed with 3×10 mL water. The resulting mixture was concentrated under reduced pressure. This resulted in CI-2 (200 mg, 62.08%) as a brown yellow oil. LCMS (ESI, m/z): [M+H]⁺ 415.2.

### Intermediate 3, 4-({7-[1-(1-Ethoxyethyl)pyrazol-4-yl]-8-isopropoxy-[1,2,41triazolo[1,5-c]pyrimidin-2-yl}amino) piperidine-1-sulfonyl chloride (CI-3)

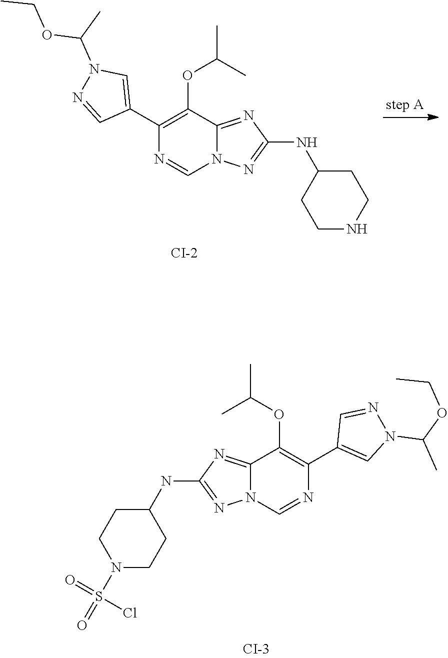

### Step A. 4-({7-[1-(1-Ethoxyethyl)pyrazol-4-yl]-8-isopropoxy-[1,2,4]triazolo[1,5-c]pyrimidin-2-yl}amino)piperidine-1-sulfonyl chloride (CI-3)

To a stirred mixture of thionyl chloride (86.10 mg, 0.723 mmol, 1.5 equiv) in dichloromethane (1 mL) were added CI-2 (200 mg, 0.482 mmol, 1 equiv) and N,N-diisopropylethylamine (187.08 mg, 1.446 mmol, 3 equiv) in portions at −78° C. under nitrogen atmosphere. The resulting mixture was stirred for 0.5 h at −78° C. under nitrogen atmosphere. The reaction was monitored by LCMS. The resulting mixture was concentrated under vacuum. The residue was purified by silica gel column chromatography, eluted with petroleum ether/ethyl acetate (1:1) to afford CI-3 (100 mg, 40.40%) as a yellow solid. LCMS (ESI, m/z): [M+H]⁺ 513.1.

### Intermediate 4, 3-Fluoro-4-f[8-isopropoxy-7-(1H-pyrazol-4-yl)-[1,2,41triazolo[1,5-c]pyrimidin-2-yl]amino}benzenesulfonyl chloride (CI-4)

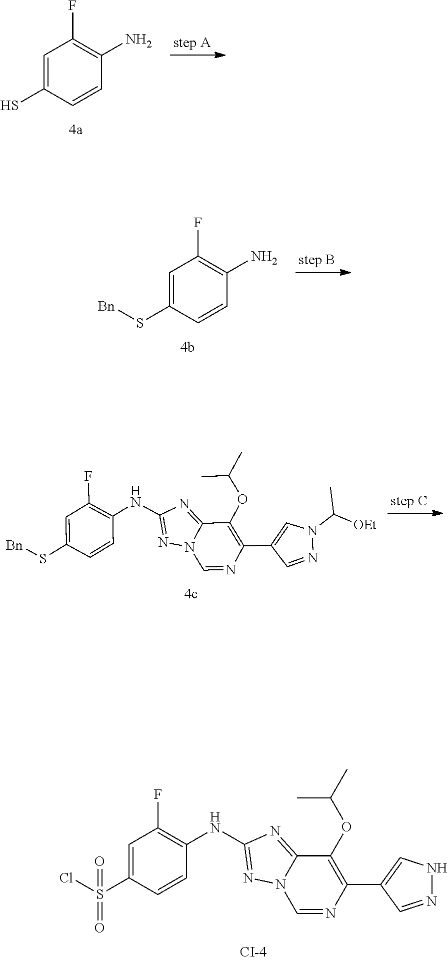 [SMILES: Nc1ccc(S)cc1F ; Nc1ccc(SCc2ccccc2)cc1F ; CCOC(C)n1cc(-c2ncn3nc(Nc4ccc(SCc5ccccc5)cc4F)nc3c2OC(C)C)cn1 ; CC(C)Oc1c(-c2cn[nH]c2)ncn2nc(Nc3ccc(S(=O)(=O)Cl)cc3F)nc12]

### Step A. 4-(Benzylsulfanyl)-2-fluoroaniline (4b)

A solution of 4a (1 g, 6.984 mmol, 1 equiv) and benzyl bromide (1.19 g, 6.984 mmol, 1 equiv) and potassium carbonate (0.97 g, 6.984 mmol, 1 equiv) in acetonitrile (10 mL) was stirred for 15 h at room temperature under nitrogen atmosphere. Desired product could be detected by LCMS. The resulting mixture was concentrated under reduced pressure. The residue was purified by silica gel column chromatography, eluted with petroleum ether/ethyl acetate (6:1) to afford 4b (900 mg, 52.47%) as a white solid. LCMS (ESI, m/z): [M+H]⁺ 234.1.

### Step B. N-[4-(Benzylsulfanyl)-2-fluorophenyl]-7-[1-(1-ethoxyethyl)pyrazol-4-yl]-8-isopropoxy-[1,2,4]triazolo[1,5-c]pyrimidin-2-amine (4c)

A solution of 4b (200 mg, 0.857 mmol, 1 equiv) and CI-1 (271.07 mg, 0.686 mmol, 0.8 equiv) and Pd₂(dba)₃ (78.50 mg, 0.086 mmol, 0.1 equiv) and Xantphos (74.41 mg, 0.129 mmol, 0.15 equiv) and cesium carbonate (558.63 mg, 1.714 mmol, 2 equiv) in 1,4-dioxane (4 mL) was stirred for 2 h at 100° C. under nitrogen atmosphere. Desired product could be detected by LCMS. The resulting mixture was concentrated under reduced pressure. The residue was purified by Prep-TLC (petroleum ether/dichloromethane/tetrahydrofuran=5/5/1) to afford 4c (230 mg, 46.54%) as a yellow solid. LCMS (ESI, m/z): [M+H]⁺ 548.2.

### Step C. 3-Fluoro-4-{[8-isopropoxy-7-(1H-pyrazol-4-yl)-[1,2,4]triazolo[1,5-c]pyrimidin-2-yl]amino}benzenesulfonyl chloride (CI-4)

A solution of 4c (280 mg, 0.511 mmol, 1 equiv) and acetic acid (122.81 mg, 2.044 mmol, 4 equiv) and water (36.84 mg, 2.044 mmol, 4 equiv) and sulfonyl chloride (276.01 mg, 2.044 mmol, 4 equiv) in dichloromethane (12 mL) was stirred for 2 h at 0° C. under nitrogen atmosphere. Desired product could be detected by LCMS. The resulting mixture was filtered. The filter cake was washed with dichloromethane (1×10 mL). The filtrate was concentrated under reduced pressure to afford CI-4 (130 mg, 53.46%) as a white solid. LCMS (ESI, m/z): [M+H]⁺ 452.1.

### Intermediate 5, 1-[3-(Bromomethyl)benzenesulfonyl]-N-{7-[1-(1-ethoxyethyl)pyrazol-4-yl]-8-isopropoxy-[1,2,4]triazolo[1,5-c]pyrimidin-2-yl}piperidin-4-amine (CI-5)

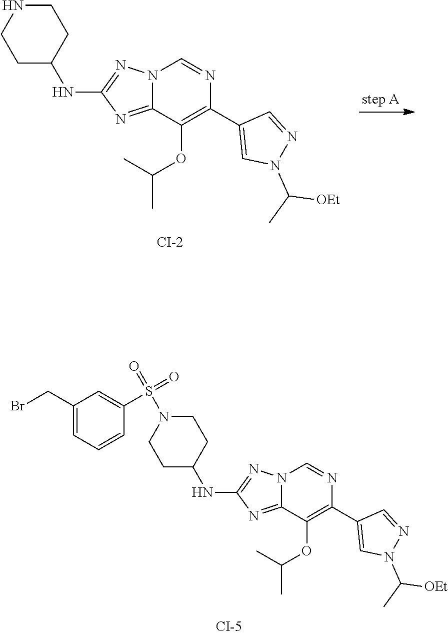 [SMILES: CCOC(C)n1cc(-c2ncn3nc(NC4CCNCC4)nc3c2OC(C)C)cn1 ; CCOC(C)n1cc(-c2ncn3nc(NC4CCN(S(=O)(=O)c5cccc(CBr)c5)CC4)nc3c2OC(C)C)cn1]

### Step A. 1-[3-(Bromomethyl)benzenesulfonyl]-N-{7-[1-(1-ethoxyethyl)pyrazol-4-yl]-8-isopropoxy-[1,2,4]triazolo[1,5-c]pyrimidin-2-yl}piperidin-4-amine (CI-5)

To a stirred solution of CI-2 (100 mg, 0.241 mmol, 1 equiv) and 3-(bromomethyl)benzenesulfonyl chloride (65.03 mg, 0.241 mmol, 1 equiv) in dichloromethane (1 mL) was added triethylamine (73.24 mg, 0.723 mmol, 3 equiv) at room temperature. The resulting mixture was stirred for 1 h at 0° C. under nitrogen atmosphere. The reaction was monitored by LCMS. The residue was purified by silica gel column chromatography, eluted with petroleum ether/ethyl acetate (1:1) to afford CI-5 (60 mg, 38.41%) as a yellow solid. LCMS (ESI, m/z): [M+H]⁺ 647.1.

### Intermediate 6, 3-[4-(7-[1-(1-Ethoxyethyl)pyrazol-4-yl]-8-isopropoxy-[1,2,4]triazolo[1,5-c]pyrimidin-2-yl]amino)-3-fluorobenzenesulfonyl]benzaldehyde (CI-6)

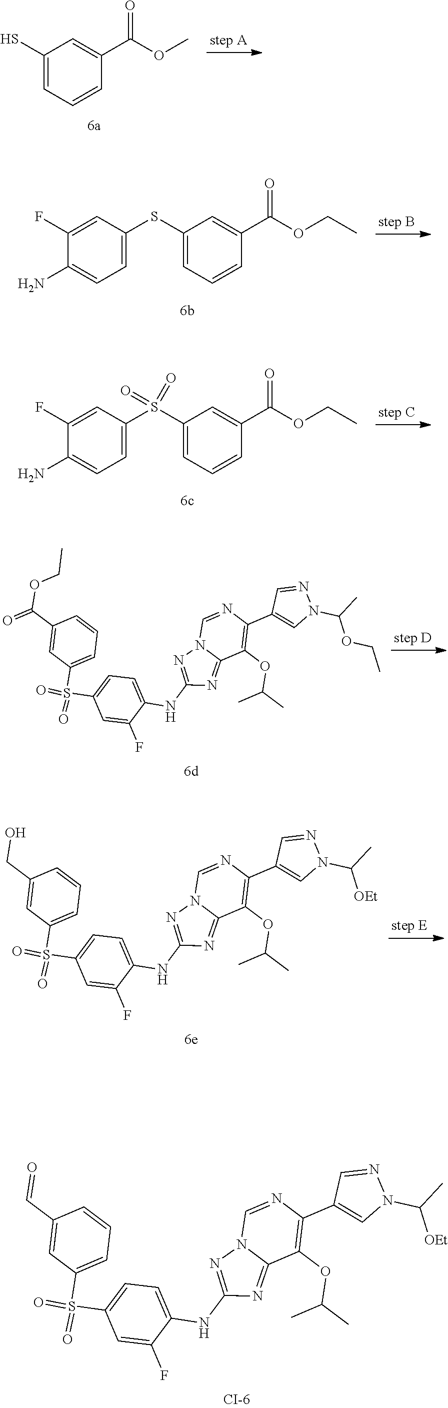 [SMILES: COC(=O)c1cccc(S)c1 ; CCOC(=O)c1cccc(Sc2ccc(N)c(F)c2)c1 ; CCOC(=O)c1cccc(S(=O)(=O)c2ccc(N)c(F)c2)c1 ; CCOC(=O)c1cccc(S(=O)(=O)c2ccc(Nc3nc4c(OC(C)C)c(-c5cnn(C(C)OCC)c5)ncn4n3)c(F)c2)c1 ; CCOC(C)n1cc(-c2ncn3nc(Nc4ccc(S(=O)(=O)c5cccc(CO)c5)cc4F)nc3c2OC(C)C)cn1 ; CCOC(C)n1cc(-c2ncn3nc(Nc4ccc(S(=O)(=O)c5cccc(C=O)c5)cc4F)nc3c2OC(C)C)cn1]

### Step A. Ethyl 3-[(4-amino-3-fluorophenyl)sulfanyl]benzoate (6b)

To a solution of 6a (3 g, 17.835 mmol, 1 equiv) and 2-fluoro-4-iodoaniline (4.44 g, 18.727 mmol, 1.05 equiv) in ethanol (50 mL) were added potassium phosphate tribasic (9.46 g, 44.588 mmol, 2.5 equiv) and Pd(PPh₃)₄(2.06 g, 1.784 mmol, 0.1 equiv). The resulting mixture was stirred for 2 h at 90° C. under a nitrogen atmosphere. Desired product could be detected by LCMS. The resulting mixture was concentrated under reduced pressure. The residue was purified by reversed-phase flash chromatography with the following conditions: column, C18 silica gel; mobile phase, 0.10% formic acid water in acetonitrile, 40% to 70% gradient in 10 min; detector, UV 254 nm. This resulted in 6b (1.5 g, 28.87%) as a brown solid. LCMS (ESI, m/z): [M+H]⁺ 292.1.

### Step B. 2-[3-(4-Amino-3-fluorobenzenesulfonyl)benzoyloxy]ethylium (6c)

To a stirred solution of 6b (1.5 g, 5.149 mmol, 1 equiv) in anhydrous dichloromethane (30 mL) was added m-chloroperoxybenzoic acid (2.67 g, 15.447 mmol, 3 equiv) at 0° C. and stirred for 1 h. Desired product could be detected by LCMS. The reaction was quenched with Na₂SO₃ aqueous at 0° C. The aqueous layer was extracted with dichloromethane (2×50 ml). The resulting mixture was concentrated under reduced pressure. The residue was purified by silica gel column chromatography, eluted with ethyl acetate/petroleum ether (⅓) to afford 6c (1.5 g, 90.10%) as a yellow solid. LCMS (ESI, m/z): [M+H]⁺ 324.0.

### Step C. Ethyl 3-[4-({7-[1-(1-ethoxyethyl)pyrazol-4-yl]-8-isopropoxy-[1,2,4]triazolo[1,5-c]pyrimidin-2-yl}amino)-3-fluorobenzenesulfonyl]benzoate (6d)

To a solution of 6c (1.5 g, 4.654 mmol, 1 equiv) and CI-1 (2207.27 mg, 5.585 mmol, 1.2 equiv) in 1,4-dioxane (0.4 mL) were added cesium carbonate (4548.71 mg, 13.962 mmol, 3 equiv) and RuPhos (434.32 mg, 0.931 mmol, 0.2 equiv), Pd₂(dba)₃ (426.15 mg, 0.465 mmol, 0.1 equiv). The resulting mixture was stirred for 1 h at 90° C. under a nitrogen atmosphere. Desired product could be detected by LCMS. The resulting mixture was concentrated under reduced pressure. The residue was purified by silica gel column chromatography, eluted with ethyl acetate/petroleum ether (¼) to afford 6d (2.5 g, 84.24%) as a yellow solid. LCMS (ESI, m/z): [M+H]⁺ 638.2.

### Step D. 3-[4-({7-[1-(1-Ethoxyethyl)pyrazol-4-yl]-8-isopropoxy-[1,2,4]triazolo[1,5-c]pyrimidin-2-yl}amino)-3-fluorobenzenesulfonyl]phenyl}methanol (6e)

To a stirred solution of 6d (1 g, 1.568 mmol, 1 equiv) in anhydrous tetrahydrofuran (15 mL) was added diisobutyl aluminium hydride (1 mol/L in n-hexane) (6 mL, 6 mmol, 3.8 equiv) at 0° C. and stirred for 1 h. Desired product could be detected by LCMS. The reaction was quenched with Anhydrous sodium sulfate at 0° C. The resulting mixture was filtered. The filtrate was concentrated under reduced pressure The residue was purified by silica gel column chromatography, eluted with ethyl acetate/petroleum ether (½) to afford 6e (750 mg, 80.29%) as a white solid. LCMS (ESI, m/z): [M+H]⁺ 596.2

### Step E. 3-[4-({7-[1-(1-Ethoxyethyl)pyrazol-4-yl]-8-isopropoxy-[1,2,4]triazolo[1,5-c]pyrimidin-2-yl}amino)-3-fluorobenzenesulfonyl]benzaldehyde (CI-6)

To a stirred solution of 6e (350 mg, 58.53%) in anhydrous dichloromethane (15.00 mL) was added manganese dioxide (525.43 mg, 6.042 mmol, 6 equiv) at 25° C. and stirred for 16 h. Desired product could be detected by LCMS. The resulting mixture was filtered and the filter cake was washed with dichloromethane (3×10 ml). The filtrate was concentrated under reduced pressure. The residue was purified by silica gel column chromatography, eluted with petroleum ether/ethyl acetate (⅔) to afford CI-6 (350 mg, 58.53%) as a white solid. LCMS (ESI, m/z): [M+H]⁺ 594.2

### Intermediate 7, 7-(1-(1-Ethoxyethyl)-1H-pyrazol-4-yl)-8-isopropoxy-N—((3R,4S)-3-methylpiperidin-4-yl]-[1,2,41 triazolo[1,5-c]pyrimidin-2-amine (CI-7)

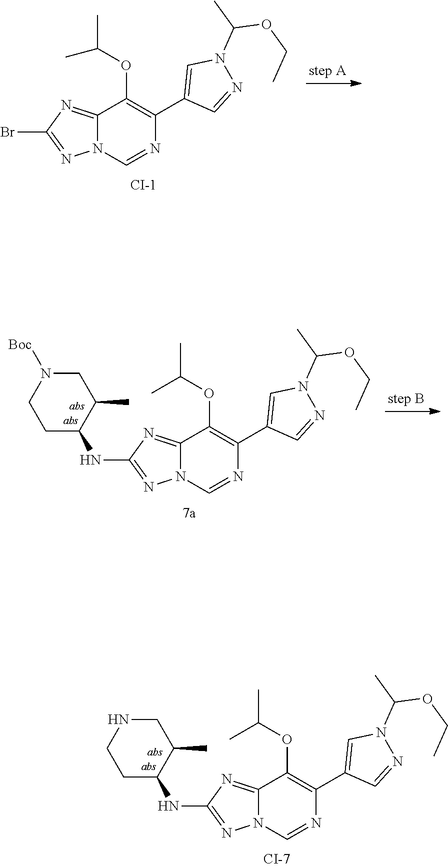 [SMILES: CCOC(C)n1cc(-c2ncn3nc(Br)nc3c2OC(C)C)cn1 ; CCOC(C)n1cc(-c2ncn3nc(N[C@H]4CCN(C(=O)OC(C)(C)C)C[C@H]4C)nc3c2OC(C)C)cn1 ; CCOC(C)n1cc(-c2ncn3nc(N[C@H]4CCNC[C@H]4C)nc3c2OC(C)C)cn1]

### Step A. Tert-butyl (3R,4S)-4-({7-[1-(1-ethoxyethyl)pyrazol-4-yl]-8-isopropoxy-[1,2,4]triazolo[1,5-c]pyrimidin-2-yl}amino)-3-methylpiperidine-1-carboxylate (7a)

To a solution of CI-1 (500 mg, 1.265 mmol, 1 equiv) and tert-butyl (3R,4S)-4-amino-3-methylpiperidine-1-carboxylate (325.32 mg, 1.518 mmol, 1.2 equiv) in 1,4-dioxane (10 mL) were added RuPhos (118.06 mg, 0.253 mmol, 0.2 equiv), Pd₂(dba)₃ (115.84 mg, 0.127 mmol, 0.1 equiv) and cesium carbonate (1236.47 mg, 3.795 mmol, 3 equiv). After stirring for 1 h at 90° C. under a nitrogen atmosphere, desired product could be detected by LCMS. The reaction was quenched with water at 25° C. The aqueous layer was extracted with EA (2×10 ml). The organic layer was concentrated under reduced pressure. The residue was purified by silica gel column chromatography, eluted with PE/EA (2:1) to afford 7a (300 mg, 44.86%) as a yellow solid. LCMS (ESI, m/z): [M+H]⁺ 529.2

### Step B. 7-(1-(1-Ethoxyethyl)-1H-pyrazol-4-yl)-8-isopropoxy-N—((3R,4S)-3-methylpiperidin-4-yl)-[1,2,4]triazolo[1,5-c]pyrimidin-2-amine (CI-7)

To a stirred mixture of 7a (275 mg, 0.520 mmol, 1 equiv) in dichloromethane (5 mL) were added trimethylsilyl trifluoromethanesulfonate (346.83 mg, 1.560 mmol, 3 equiv) and triethylamine (315.83 mg, 3.120 mmol, 6 equiv) dropwise at 0° C. The resulting mixture was stirred for 30 min at room temperature. The reaction was monitored by LCMS. The resulting mixture was extracted with dichloromethane (3×20 mL), dried over anhydrous sodium sulfate. After filtration, the filtrate was concentrated under reduced pressure. The crude product was used in the next step directly without further purification. LCMS (ESI, m/z): [M+H]⁺ 428.2

### Intermediate 8, 3-[3-Methyl-2-oxo-4-(piperidin-4-yl)-1,3-benzodiazol-1-yl]piperidine-2,6-dione (13-3). 3-[3-Methyl-2-oxo-4-(piperidin-4-yl)-1,3-benzodiazol-1-yl]piperidine-2,6-dione (CI-8)

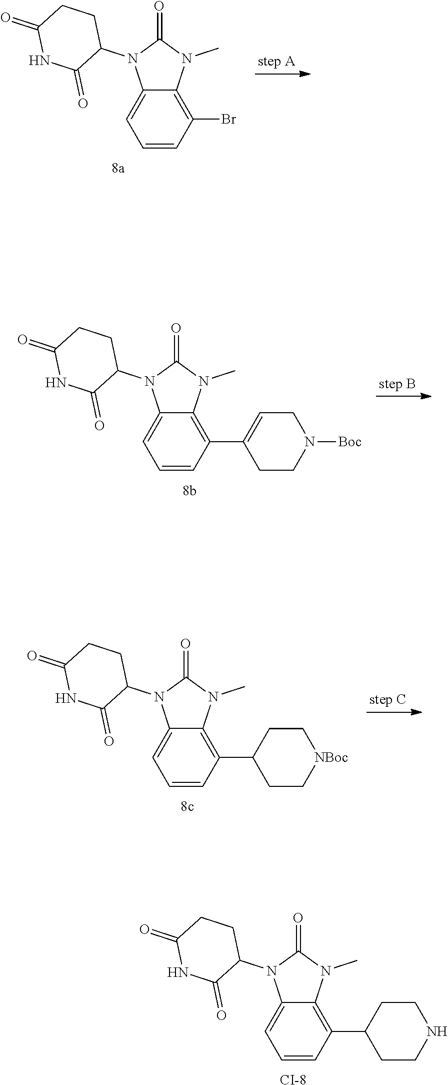

### Step A. Tert-butyl 4-[1-(2,6-dioxopiperidin-3-yl)-3-methyl-2-oxo-1,3-benzodiazol-4-yl]-3,6-dihydro-2H-pyridine-1-carboxylate (8b)

To a stirred mixture of 3-(4-bromo-3-methyl-2-oxo-1,3-benzodiazol-1-yl)piperidine-2,6-dione (8a, 1 g, 2.957 mmol, 1 equiv) and tert-butyl 4-(4,4,5,5-tetramethyl-1,3,2-dioxaborolan-2-yl)-3,6-dihydro-2H-pyridine-1-carboxylate (1.19 g, 3.844 mmol, 1.3 equiv) in dioxane (10 mL) and water (1 mL) was added Xphos Pd G3 (0.38 g, 0.444 mmol, 0.15 equiv) and potassium phosphate tribasic (1.88 g, 8.871 mmol, 3 equiv). The resulting mixture was stirred for 3 h at 60° C. The resulting mixture was concentrated under reduced pressure. The residue was purified by silica gel column chromatography, eluted with dichloromethane/methanol (20:1) to afford 8b (1.1 g, 80.22%) as a white solid. LCMS (ESI, m/z): [M+H]⁺ 441.2

### Step B. Tert-butyl 4-[1-(2,6-dioxopiperidin-3-yl)-3-methyl-2-oxo-1,3-benzodiazol-4-yl]piperidine-1-carboxylate (Sc)

To a stirred mixture of 8b (1.1 g, 2.497 mmol, 1 equiv) in tetrahydrofuran (13 mL) was added Pd/C (0.55 g, 5.169 mmol, 2.07 equiv) and Pd(OH)₂/C (0.55 g, 3.920 mmol, 1.57 equiv). The resulting mixture was stirred for 3 h at 50° C. under hydrogen atmosphere. The resulting mixture was filtered, the filter cake was washed with tetrahydrofuran. The filtrate was concentrated under reduced pressure to afford 8c (1 g, 84.16%) as a white solid. LCMS (ESI, m/z): [M+H]⁺ 443.2.

### Step C. 3-[3-Methyl-2-oxo-4-(piperidin-4-yl)-1,3-benzodiazol-1-yl]piperidine-2,6-dione (CI-8)

To a stirred mixture of 8c (1 g, 2.260 mmol, 1 equiv) in dichloromethane (3 mL) was added 4M HCl(gas)in 1,4-dioxane (6 mL). The resulting mixture was stirred for 1 h at 25° C. The resulting mixture was concentrated under reduced pressure to afford CI-8 (850 mg, crude) as a white solid. The crude product was used in the next step directly without further purification. LCMS (ESI, m/z): [M+H]⁺=343.2.

### Intermediate 9, 3-(3-Methyl-4-(1-(3-(methylamino)propyl)piperidin-4-yl)-2-oxo-2,3-dihydro-1H-benzo[d]imidazol-1-yl)piperidine-2,6-dione (CI-9)

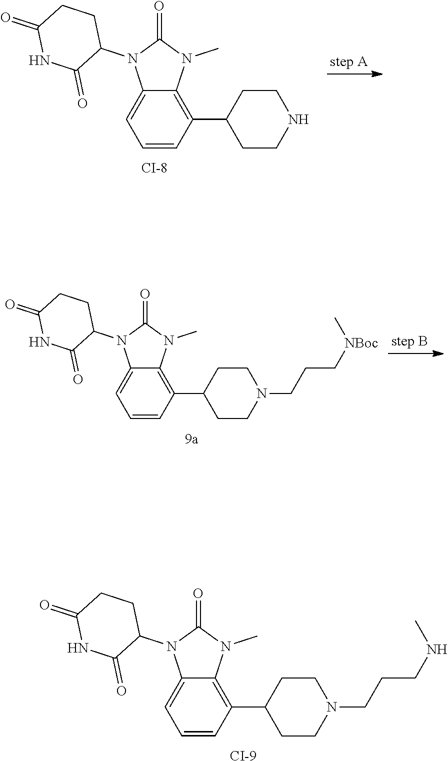

### Step A. Tert-butyl N-(3-{4-[1-(2,6-dioxopiperidin-3-yl)-3-methyl-2-oxo-1,3-benzodiazol-4-yl]piperidin-1-yl}propyl)-N-methylcarbamate (9a)

To a stirred mixture of CI-8 (500 mg, 1.460 mmol, 1 equiv) and tert-butyl N-methyl-N-(3-oxopropyl)carbamate (546.84 mg, 2.920 mmol, 2 equiv) in tetrahydrofuran (5 mL) and N,N-dimethylformamide (1 mL) was added acetic acid (0.2 mL). The resulting mixture was stirred for 0.5 h at 25° C. Then sodium triacetoxyborohydride (810.87 mg, 3.825 mmol, 2.62 equiv) was added to the mixture. The resulting mixture was stirred for 1 h at 25° C. The resulting mixture was concentrated under reduced pressure. The reaction was quenched by the addition of water (10 mL). The resulting mixture was extracted with EA. The combined organic layers were dried over anhydrous sodium sulfate. After filtration, the filtrate was concentrated under reduced pressure. The residue was purified by silica gel column chromatography, eluted with methanol/dichloromethane (1:30) to afford 9a (400 mg, 52.26%) as a white solid. LCMS (ESI, m/z): [M+H]⁺ 514.3.

### Step B. 3-(3-Methyl-4-(1-(3-(methylamino)propyl)piperidin-4-yl)-2-oxo-2,3-dihydro-1H-benzo[d]imidazol-1-yl)piperidine-2,6-dione (CI-9)

To a stirred mixture of 9a (71 mg, 0.138 mmol, 1 equiv) in acetonitrile (5 mL) was added 4M HCl(gas)in 1,4-dioxane (0.5 mL). The resulting mixture was stirred for 1 h at 25° C. The resulting mixture was concentrated under reduced pressure to afford CI-9 (65 mg, crude) as a white solid. The crude product was used in the next step directly without further purification. LCMS (ESI, m z): [M+H]=414.2.

### Intermediate 10, 1-[1-methyl-6-(piperidin-4-yl)indazol-3-yl}-1,3-diazinane-2,4-dione (CI-10)

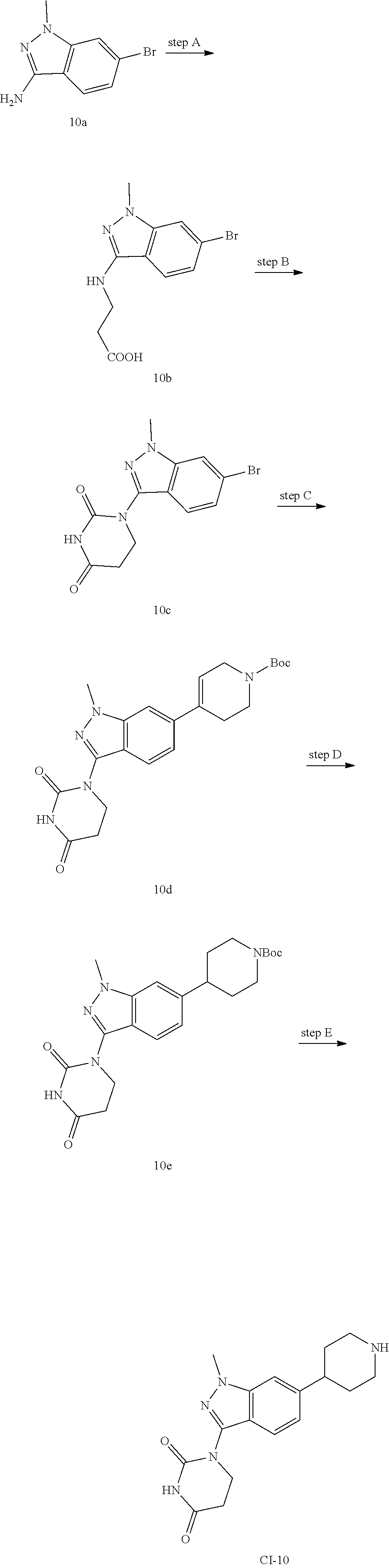 [SMILES: Cn1nc(N)c2ccc(Br)cc21 ; Cn1nc(NCCC(=O)O)c2ccc(Br)cc21 ; Cn1nc(N2CCC(=O)NC2=O)c2ccc(Br)cc21 ; Cn1nc(N2CCC(=O)NC2=O)c2ccc(C3=CCN(C(=O)OC(C)(C)C)CC3)cc21 ; Cn1nc(N2CCC(=O)NC2=O)c2ccc(C3CCN(C(=O)OC(C)(C)C)CC3)cc21 ; Cn1nc(N2CCC(=O)NC2=O)c2ccc(C3CCNCC3)cc21]

### Step A. 3-[(6-Bromo-1-methylindazol-3-yl)amino]propanoic acid (10b)

To a stirred solution of 10a (5 g, 22.116 mmol, 1 equiv) and acetic acid (3.15 g, 52.415 mmol, 2.37 equiv) in water (8 mL) was added acrylic acid (1.59 g, 22.116 mmol, 1 equiv) dropwise at room temperature. The resulting mixture was stirred for overnight at 100° C. under nitrogen atmosphere. Desired product could be detected by LCMS. The mixture was basified to pH 7 with 6N HCl (aq.). The aqueous layer was extracted with ethyl acetate (3×50 mL). The combined organic layers were dried over anhydrous sodium sulfate. After filtration, the filtrate was concentrated under reduced pressure. The residue was purified by silica gel column chromatography, eluted with dichloromethane/methanol (10:1) to afford 10b (1.84 g, 25.64%) as a yellow solid. LCMS (ESI, m/z): [M+H]⁺=298.0

### Step B. 1-(6-Bromo-1-methylindazol-3-yl)-1,3-diazinane-2,4-dione (10c)

To a stirred solution of 10b (900 mg, 3.019 mmol, 1 equiv) in acetic acid (9 mL) was added urea (906.46 mg, 15.095 mmol, 5 equiv) in portions at room temperature under nitrogen atmosphere. The resulting mixture was stirred for overnight at 120° C. under nitrogen atmosphere. Desired product could be detected by LCMS. The resulting mixture was concentrated under reduced pressure. The residue was purified by silica gel column chromatography, eluted with dichloromethane/methanol (10:1) to afford 10c (330 mg, 32.44%) as a yellow solid. LCMS (ESI, m/z): [M+H]⁺ 323.0

### Step C. Tert-butyl 4-[3-(2,4-dioxo-1,3-diazinan-1-yl)-1-methylindazol-6-yl]-3,6-dihydro-2H-pyridine-1-carboxylate (10d)

To a solution of 10c (310 mg, 0.959 mmol, 1 equiv) and tert-butyl 4-(4,4,5,5-tetramethyl-1,3,2-dioxaborolan-2-yl)-3,6-dihydro-2H-pyridine-1-carboxylate (444.94 mg, 1.438 mmol, 1.5 equiv) in dioxane (4 mL) and water (0.4 mL) were added Xphos Pd G3 (81.20 mg, 0.096 mmol, 0.1 equiv) and potassium phosphate tribasic (610.88 mg, 2.877 mmol, 3 equiv). After stirring for 3 h at 60° C. under a nitrogen atmosphere, the resulting mixture was concentrated under reduced pressure. The resulting mixture was concentrated under reduced pressure. The residue was purified by silica gel column chromatography, eluted with petroleum ether/ethyl acetate (1:1) to afford 10d (320 mg, 53.62%) as a white solid. LCMS (ESI, m/z): [M+H]⁺ 426.2

### Step D. Tert-butyl 4-[3-(2,4-dioxo-1,3-diazinan-1-yl)-1-methylindazol-6-yl]piperidine-1-carboxylate (10e)

To a solution of 10d (320 mg, 0.752 mmol, 1 equiv) in CF₃CwaterH (20 mL) was added Pd/C (160.07 mg, 1.504 mmol, 2 equiv) in a pressure tank. The mixture was hydrogenated at room temperature under 30 psi of hydrogen pressure for 3 h, filtered through a Celite pad and concentrated under reduced pressure. The residue was purified by silica gel column chromatography, eluted with petroleum ether/ethyl acetate (1:1) to afford 10e (240 mg, 71.06%) as a white solid. LCMS (ESI, m/z): [M+H]⁺ 428.2

### Step E. 1-[1-Methyl-6-(piperidin-4-yl)indazol-3-yl]-1,3-diazinane-2,4-dione (CI-10)

To a stirred solution of 10e (50 mg, 0.117 mmol, 1 equiv) in acetonitrile (2.5 mL) was added 4M HCl (gas) in 1,4-dioxane (0.5 mL) dropwise at 0° C. The resulting mixture was stirred for 30 min at 0° C. Desired product could be detected by LCMS. The mixture was basified to pH 7 with saturated sodium bicarbonate (aq.). The aqueous layer was extracted with ethyl acetate (3×30 mL). The combined organic layers were dried over anhydrous sodium sulfate. After filtration, the filtrate was concentrated under reduced pressure. The crude product was used in the next step directly without further purification. LCMS (ESI, m/z): [M+H]⁺ 328.1

### Intermediate 11, 3-(1-Oxo-5-(2,6-diazaspiro[3.3]heptan-2-yl)isoindolin-2-yl)piperidine-2,6-dione (CI-11)

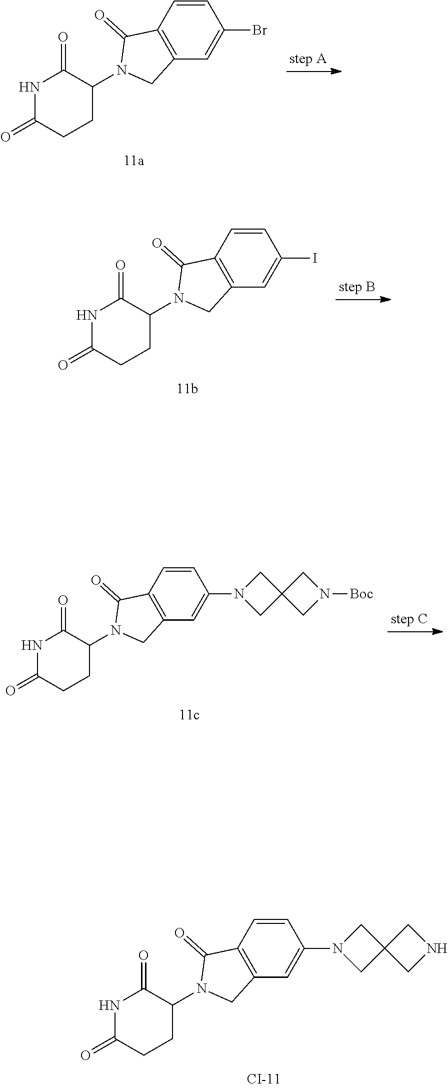 [SMILES: O=C1CCC(N2Cc3cc(Br)ccc3C2=O)C(=O)N1 ; O=C1CCC(N2Cc3cc(I)ccc3C2=O)C(=O)N1 ; CC(C)(C)OC(=O)N1CC2(C1)CN(c1ccc3c(c1)CN(C1CCC(=O)NC1=O)C3=O)C2 ; O=C1CCC(N2Cc3cc(N4CC5(CNC5)C4)ccc3C2=O)C(=O)N1]

### Step A. 3-(5-Iodo-1-oxo-3H-isoindol-2-yl) piperidine-2,6-dione (11b)

To a solution of 11a (11 g, 34.040 mmol, 1 equiv) in 1,4-dioxane (110 mL) was added (1R,2R)-N1,N2-dimethylcyclohexane-1,2-diamine (1.94 g, 13.616 mmol, 0.4 equiv), sodium iodide (20.424 g, 136.160 mmol, 4 equiv), cuprous iodide (2.59 g, 13.616 mmol, 0.4 equiv) at room temperature. The mixture was stirred for overnight at 125° C. Desired product could be detected by LCMS. The resulting mixture was concentrated under reduced pressure. The residue was purified by silica gel column chromatography, eluted with dichloromethane/methanol (10/1) to afford 11b (9.1 g, 72.22%) as a dark green solid. LCMS (ESI, m/z): [M+H]⁺ 371.0.

### Step B. Tert-butyl 6-(2-(2,6-dioxopiperidin-3-yl)-1-oxoisoindolin-5-yl)-2,6-diazaspiro[3.3]heptane-2-carboxylate (11c)

To a mixture of 11b (200 mg, 0.540 mmol, 1 equiv) and tert-butyl 2,6-diazaspiro[3.3]heptane-2-carboxylate (107.13 mg, 0.540 mmol, 1 equiv) and Pd-PEPPSI-IPentCl 3-chloropyridine (39.35 mg, 0.054 mmol, 0.1 equiv) in dioxane (10 mL) were added cesium carbonate (352.10 mg, 1.080 mmol, 2 equiv) at 25° C. under N₂. The resulting mixture was stirred for 1 h at 80° C. Desired product could be detected by LCMS. The mixture was concentrated under vacuum. The resulting mixture was diluted with water (5 mL) and extracted with ethyl acetate (3×10 mL). The combined organic layers were dried over anhydrous sodium sulfate and concentrated under reduced pressure. The residue was purified by reversed-phase Combi-Flash, eluted with acetonitrile:water (0.5% ammonium bicarbonate)=5%-60%. This resulted in 11c (100 mg, 35.71%) as a yellow solid. LCMS (ESI, m/z): [M+H]⁺ 441.2.

### Step C. 3-(1-Oxo-5-(2,6-diazaspiro[3.3]heptan-2-yl)isoindolin-2-yl)piperidine-2,6-dione (CI-11)

To a stirred solution of 11c (100 mg, 0.227 mmol, 1 equiv) in dichloromethane (10 mL) was added trifluoroacetic acid (776.55 mg, 6.810 mmol, 30 equiv) at 0° C. under N₂. The resulting mixture was stirred for 1 h at 25° C. Desired product could be detected by LCMS. The mixture was concentrated under vacuum. The resulting mixture was diluted with water (5 mL) and extracted with ethyl acetate (3×10 mL). The combined organic layers were dried over anhydrous sodium sulfate and concentrated under reduced pressure. The residue was purified by reversed-phase Combi-Flash to afford CI-11 (65 mg, 63.09%) as a yellow solid. LCMS (ESI, m/z): [M+H]⁺ 341.2.

### Intermediate 12, 3-[6-Fluoro-1-oxo-5-(piperazin-1-yl)-3H-isoindol-2-yl}piperidine-2,6-dione (CI-12)

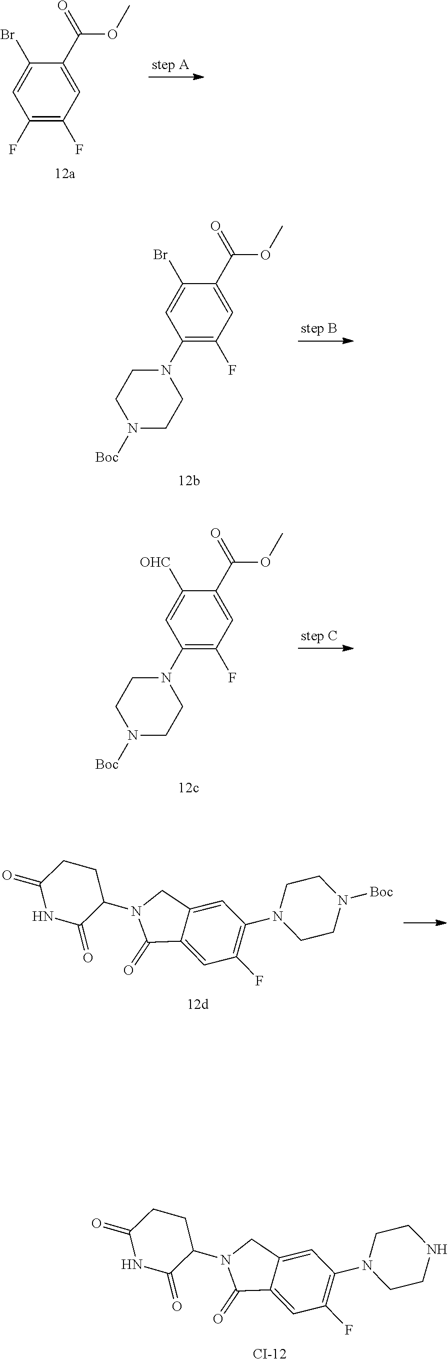 [SMILES: COC(=O)c1cc(F)c(F)cc1Br ; COC(=O)c1cc(F)c(N2CCN(C(=O)OC(C)(C)C)CC2)cc1Br ; COC(=O)c1cc(F)c(N2CCN(C(=O)OC(C)(C)C)CC2)cc1C=O ; CC(C)(C)OC(=O)N1CCN(c2cc3c(cc2F)C(=O)N(C2CCC(=O)NC2=O)C3)CC1 ; O=C1CCC(N2Cc3cc(N4CCNCC4)c(F)cc3C2=O)C(=O)N1]

### Step A. Tert-butyl 4-(5-bromo-2-fluoro-4-(methoxycarbonyl)phenyl)piperazine-1-carboxylate (12b)

To a stirred mixture of 12a (2 g, 7.967 mmol, 1 equiv) and tert-butyl piperazine-1-carboxylate (1.48 g, 7.967 mmol, 1 equiv) in N,N-dimethylformamide (10 mL) were added potassium carbonate (3.30 g, 23.901 mmol, 3 equiv) in portions at room temperature under nitrogen atmosphere. The resulting mixture was stirred for overnight at 80° C. under nitrogen atmosphere. Desired product could be detected by LCMS. The resulting mixture was diluted with water (20 mL). The resulting mixture was extracted with ethyl acetate (3×20 mL). The combined organic layers were washed with water (3×10 mL), dried over anhydrous sodium sulfate. After filtration, the filtrate was concentrated under reduced pressure. The residue was purified by silica gel column chromatography, eluted with petroleum ether/ethyl acetate (6:1) to afford 12b (2.3 g, 69.18%) as a brown solid. LCMS (ESI, m/z): [M+H]⁺ 417.1.

### Step B. Tert-butyl 4-[2-fluoro-5-formyl-4-(methoxycarbonyl)phenyl]piperazine-1-carboxylate (12c)

To a stirred mixture of 12b (2.3 g, 5.512 mmol, 1 equiv) and 2-isocyano-2-methylpropane (0.92 g, 11.024 mmol, 2 equiv) in N,N-dimethylformamide (20 mL) were added Palladium acetate (0.12 g, 0.551 mmol, 0.1 equiv) and triethylsilane (0.06 g, 0.551 mmol, 0.1 equiv) and tricyclohexylphosphane (4.64 g, 16.536 mmol, 3 equiv) and sodium carbonate (1.75 g, 16.536 mmol, 3 equiv) room temperature under nitrogen atmosphere. The resulting mixture was stirred for overnight at 35° C. under nitrogen atmosphere. Desired product could be detected by LCMS. The resulting mixture was diluted with water (20 mL). The resulting mixture was extracted with ethyl acetate (3×20 mL). The combined organic layers were washed with water (3×10 mL), dried over anhydrous sodium sulfate. After filtration, the filtrate was concentrated under reduced pressure. The residue was purified by silica gel column chromatography, eluted with petroleum ether/ethyl acetate (5:1) to afford 12c (1.3 g, 64.37%) as a brown solid. LCMS (ESI, m/z): [M+H]⁺ 367.2.

### Step C. Tert-butyl 4-(2-(2,6-dioxopiperidin-3-yl)-6-fluoro-1-oxoisoindolin-5-yl)piperazine-1-carboxylate (12d)

To a stirred mixture of 3-aminopiperidine-2,6-dione (0.68 g, 5.322 mmol, 1.5 equiv) and 12c (1.3 g, 3.548 mmol, 1 equiv) in methanol (10 mL) were added acetic acid (0.64 g, 10.657 mmol, 3.00 equiv) at room temperature under air atmosphere. The resulting mixture was stirred for 0.5 h at room temperature under nitrogen atmosphere. To the above mixture was added sodium cyanoborohydride (0.33 g, 5.322 mmol, 1.5 equiv) in portions over 2 min at room temperature. The resulting mixture was stirred for additional overnight at room temperature. Desired product could be detected by LCMS. The residue was purified by silica gel column chromatography, eluted with petroleum ether/ethyl acetate (2:1) to afford 12d (1.1 g, 69.44%) as an off-white solid. LCMS (ESI, m/z): [M+H]⁺ 447.2.

### Step D. 3-[6-Fluoro-1-oxo-5-(piperazin-1-yl)-3H-isoindol-2-yl]piperidine-2,6-dione (CI-12)

To a stirred solution of 12d (60 mg, 0.134 mmol, 1 equiv) in acetonitrile (1.5 mL) was added 4 M HCl (gas) in 1,4-dioxane (0.3 mL) dropwise at 0° C. under nitrogen atmosphere. The resulting mixture was stirred for 1 h at 0° C. Desired product could be detected by LCMS. The resulting mixture was concentrated under reduced pressure. This resulted in CI-12 (50 mg, crude) as a white solid. The crude product was used in the next step directly without further purification. LCMS (ESI, m/z): [M+H]⁺ 347.1.

### Intermediate 13, 3-[5-(Azetidin-3-yl)-1-oxo-3H-isoindol-2-yl}piperidine-2,6-dione (CI-13)

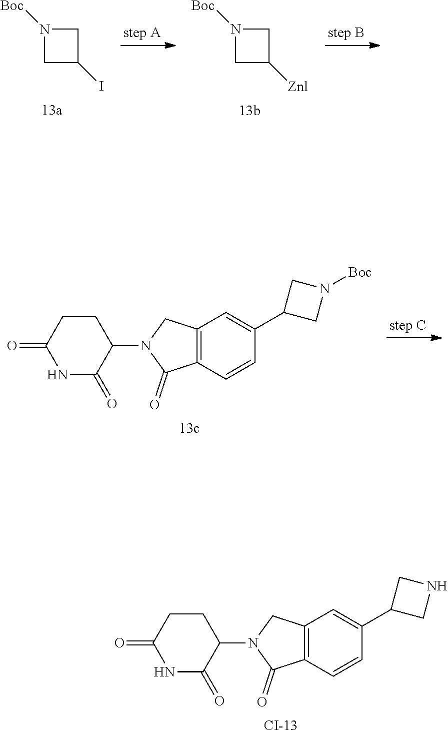

### Step A. (1-(Tert-butoxycarbonyl)azetidin-3-yl)zinc(II) iodide (13b)

To a stirred solution of 13a (2 g, 7.064 mmol, 1 equiv) and Zinc powder (923.76 mg, 14.128 mmol, 2 equiv) in tetrahydrofuran (60 mL) were added trimethylchlorosilane (307 mg, 2.824 mmol, 0.4 equiv) and 1,2-bromoethane (525.6 mg, 2.824 mmol, 0.4 equiv) in portions at room temperature. The resulting mixture was stirred for 2 h at 60° C. under nitrogen atmosphere. Desired product could be detected by TLC (petroleum ether/ethyl acetate=1:1, Rf=0.5). The resulting mixture was used in the next step directly without further purification.

### Step B. Tert-butyl 3-[2-(2,6-dioxopiperidin-3-yl)-1-oxo-3H-isoindol-5-yl]azetidine-1-carboxylate (13c)

To the above mixture were added 13b (400 mg, 1.08 mmol, 1.00 equiv), Pd(dppf)C₁₋₂ (79.08 mg, 0.108 mmol, 0.1 equiv) and cuprous iodide (20.58 mg, 0.108 mmol, 0.1 equiv) at room temperature. The resulting mixture was stirred for 1 h at 80° C. under nitrogen atmosphere. Desired product could be detected by LCMS. The resulting mixture was extracted with ethyl acetate (3×10 mL). The combined organic layers were washed with brine (10 mL), dried over anhydrous sodium sulfate. After filtration, the filtrate was concentrated under reduced pressure. The residue was purified by reversed-phase flash chromatography with the following conditions: column, C18 silica gel; mobile phase, acetonitrile in water (0.1% formic acid), 10% to 50% gradient in 30 min. The resulting mixture was concentrated under reduced pressure to afford 13c (120 mg, 27.80%) as a brown solid. LCMS (ESI, m/z): [M+H]⁺ 400.2.

### Step C. 3-[5-(Azetidin-3-yl)-1-oxo-3H-isoindol-2-yl]piperidine-2,6-dione (CI-13)

To a stirred solution of 13c (150 mg, 0.336 mmol, 1 equiv) in anhydrous acetonitrile (2.5 mL) was added 4M HCl (gas) in 1,4-dioxane (0.5 mL) at 0° C. and stirred for 1 h. Desired product could be detected by LCMS. The resulting mixture was concentrated under reduced pressure. The resulting mixture was used in the next step directly without further purification. This resulted in CI-13 (150 mg, crude) as a white solid. The crude product was used in the next step directly without further purification. LCMS (ESI, m/z): [M+H]⁺ 300.1

### Intermediate 14, 3-{3-Methyl-2-oxo-4-[1-(piperidin-4-ylmethyl)piperidin-4-yl}-1,3-benzodiazol-1-yl}piperidine-2,6-dione (CI-14)

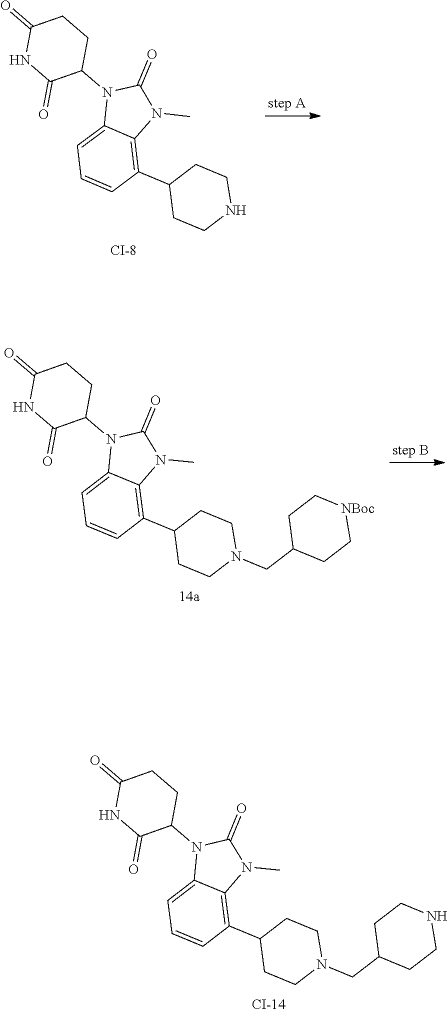 [SMILES: Cn1c(=O)n(C2CCC(=O)NC2=O)c2cccc(C3CCNCC3)c21 ; Cn1c(=O)n(C2CCC(=O)NC2=O)c2cccc(C3CCN(CC4CCN(C(=O)OC(C)(C)C)CC4)CC3)c21 ; Cn1c(=O)n(C2CCC(=O)NC2=O)c2cccc(C3CCN(CC4CCNCC4)CC3)c21]

### Step A. Tert-butyl 4-({4-[1-(2,6-dioxopiperidin-3-yl)-3-methyl-2-oxo-1,3-benzodiazol-4-yl]piperidin-1-yl}methyl)piperidine-1-carboxylate (14a)

To a solution of CI-8 (600 mg, 1.752 mmol, 1 equiv) and tert-butyl 4-(bromomethyl)piperidine-1-carboxylate (974.97 mg, 3.504 mmol, 2 equiv) in acetonitrile (5 mL) was added sodium iodide (262.67 mg, 1.752 mmol, 1 equiv) and potassium carbonate (1453.09 mg, 10.512 mmol, 6 equiv) at room temperature. The resulting mixture was stirred for overnight at 70° C. The residue was purified by Prep-TLC (dichloromethane/methanol=30/1) to afford 14a (350 mg, 37.01%) as a yellow solid. LCMS (ESI, m/z): [M+H]⁺ 540.3

### Step B. 3-{3-Methyl-2-oxo-4-[1-(piperidin-4-ylmethyl)piperidin-4-yl]-1,3-benzodiazol-1-yl}piperidine-2,6-dione (CI-14)

To a stirred mixture of 14a (65 mg, 0.120 mmol, 1 equiv) in acetonitrile (5 mL) was added 4M HCl(gas)in 1,4-dioxane (1 mL) at 0° C. The resulting mixture was stirred for 1 h at 25° C. The resulting mixture was concentrated under reduced pressure to afford CI-14 (60 mg, crude) as a white solid. The crude product was used in the next step directly without further purification. LCMS (ESI, m/z): [M+H]⁺ 440.3

### Intermediate 15, 1-[2-(2,6-Dioxopiperidin-3-yl)-1-oxo-3H-isoindol-5-yl}piperidine-4-carbaldehyde (CI-15)

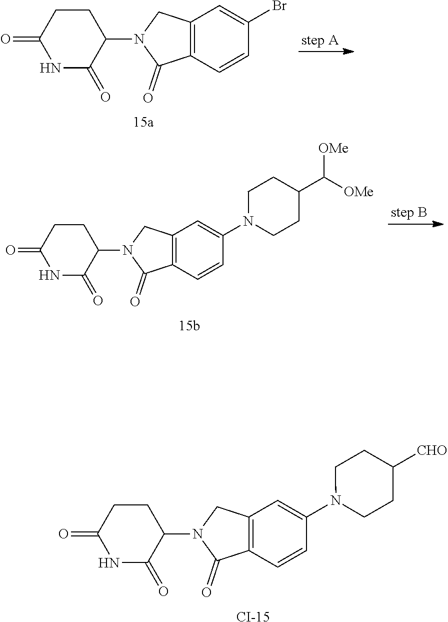

### Step A. 3-{5-[4-(Dimethoxymethyl)piperidin-1-yl]-1-oxo-3H-isoindol-2-yl}piperidine-2,6-dione (15b)

To a mixture of 15a (1 g, 3.095 mmol, 1 equiv) and 4-(dimethoxymethyl)piperidine (1.23 g, 7.738 mmol, 2.5 equiv) in dimethyl sulfoxide (10 mL) was added Pd-PEPPSI-IPentCl 3-chloropyridine (245.30 mg, 0.310 mmol, 0.1 equiv) and cesium carbonate (2.02 g, 6.190 mmol, 2 equiv). The resulting mixture was stirred for 1 h at 80° C. Desired product could be detected by LCMS. The reaction was quenched by the addition of water (5 mL). The resulting mixture was extracted with ethyl acetate. The combined organic layers were dried over anhydrous sodium sulfate. After filtration, the filtrate was concentrated under reduced pressure. The residue was purified by silica gel column chromatography, eluted with petroleum ether/ethyl acetate (1:1) to afford 15b (440 mg, 35.42%) as a white solid. LCMS (ESI, m/z): [M+H]⁺ 402.2

### Step B. 1-[2-(2,6-dioxopiperidin-3-yl)-1-oxo-3H-isoindol-5-yl]piperidine-4-carbaldehyde (CI-15)

To a stirred mixture of 15b (440 mg, 1.096 mmol, 1 equiv) in tetrahydrofuran (12.6 mL) was added 2 N HCl (0.76 mL, 1.520 mmol, 1.39 equiv). The resulting mixture was stirred for 2 h at room temperature. Desired product could be detected by LCMS. The reaction was quenched by the addition of saturated sodium bicarbonate (aq.) (10 mL). The resulting mixture was extracted with ethyl acetate. The combined organic layers were dried over anhydrous sodium sulfate. After filtration, the filtrate was concentrated under reduced pressure to afford CI-15 (300 mg, crude) as a white solid. LCMS (ESI, m/z): [M+H]⁺ 356.2

### Intermediate 16, 3-(1-oxo-5-(piperazin-1-yl)isoindolin-2-yl)piperidine-2,6-dione (CI-1

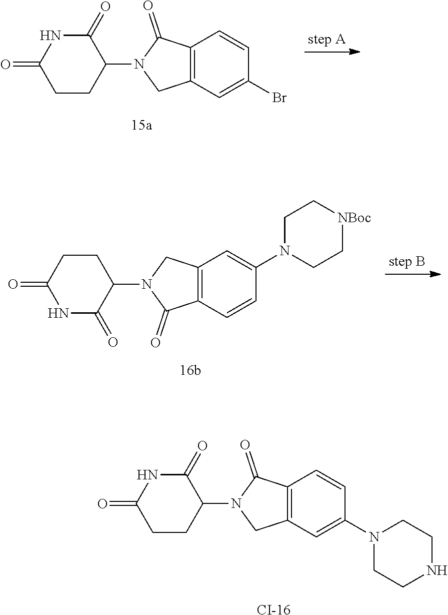 [SMILES: O=C1CCC(N2Cc3cc(Br)ccc3C2=O)C(=O)N1 ; CC(C)(C)OC(=O)N1CCN(c2ccc3c(c2)CN(C2CCC(=O)NC2=O)C3=O)CC1 ; O=C1CCC(N2Cc3cc(N4CCNCC4)ccc3C2=O)C(=O)N1 ; "CI-16"]

### Step A: tert-butyl 4-[2-(2,6-dioxopiperidin-3-yl)-1-oxo-3H-isoindol-5-yl]piperazine-1-carboxylate (16a)

To a stirred solution of 15a (2.59 g, 13.926 mmol, 1.5 equiv) in N,N-dimethylformamide (40 mL) was added cesium carbonate (9.07 g, 27.852 mmol, 3 equiv) and RuPhos Palladacycle Gen.3 (1.55 g, 1.857 mmol, 0.2 equiv) at room temperature under nitrogen atmosphere. The resulting mixture was stirred for overnight at 95° C. under nitrogen atmosphere. The residue was washed with water (10×5 mL). The resulting mixture was extracted with ethyl acetate (300 mL). The combined organic layers were washed with brine (5×50 mL), dried over anhydrous sodium sulfate. After filtration, the filtrate was concentrated under reduced pressure. The residue was purified by silica gel column chromatography, eluted with petroleum ether/acetic ester (1:1) to afford 16a (895 mg, 18.00%) as a light yellow solid. LCMS (ESI, m/z): [M+H]⁺ 429.2

### Step B: 3-(1-oxo-5-(piperazin-1-yl)isoindolin-2-yl)piperidine-2,6-dione (CI-16)

To a stirred solution of 16a (120 mg, 0.280 mmol, 1 equiv) in anhydrous acetonitrile (2.5 mL) was added HCl(gas)in 1,4-dioxane (0.5 mL) at 0° C. and stirred for 3 h. The reaction progress was monitored by LCMS. Desired product could be detected by LCMS. The mixture was neutralized to pH 7 with triethylamine. The resulting mixture was concentrated under reduced pressure. The crude product was used in the next step directly without further purification. LCMS (ESI, m/z): [M+H]⁺ 329.1

### Intermediate 17, 4-(3-bromobenzenesulfonyl)-2-fluoroaniline (CI-17)

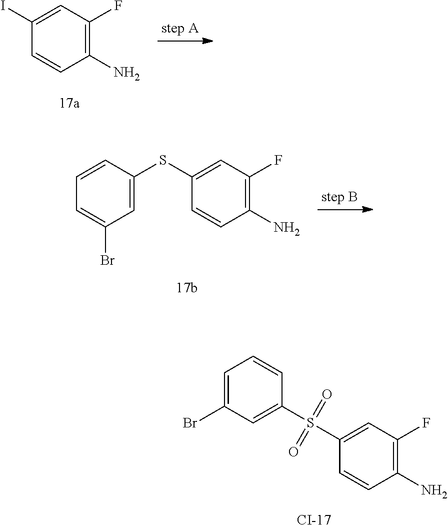 [SMILES: Nc1ccc(I)cc1F ; Nc1ccc(Sc2cccc(Br)c2)cc1F ; Nc1ccc(S(=O)(=O)c2cccc(Br)c2)cc1F ; "CI-17"]

### Step A: 4-[(3-bromophenyl)sulfanyl]-2-fluoroaniline (17b)

To a solution of 17a (4 g, 16.876 mmol, 1 equiv) and 3-bromobenzenethiol (3.35 g, 17.720 mmol, 1.05 equiv) in ethanol (66 mL) were added Pd(PPh₃)₄(1.95 g, 1.688 mmol, 0.1 equiv) and sodium tert-butanol (4.05 g, 42.190 mmol, 2.5 equiv). After stirring for 2 h at 90° C. under a nitrogen atmosphere, the resulting mixture was concentrated under reduced pressure. The residue was purified by reversed-phase flash chromatography with the following conditions: column, C18 silica gel; mobile phase, 0.1% trifluoroacetic acid water in acetonitrile, 70% to 100% gradient in 10 min; detector, UV 254 nm. This resulted in 4-[(3-bromophenyl)sulfanyl]-2-fluoroaniline (3.5 g, 69.55%) as a yellow oil. LCMS (ESI, m/z): [M+H]⁺ 297.9

### Step B: 4-(3-bromobenzenesulfonyl)-2-fluoroaniline (CI-17)

A solution of 17b (2.5 g, 8.384 mmol, 1 equiv) and m-chloroperoxybenzoic acid (4.34 g, 25.152 mmol, 3.0 equiv) in dichloromethane was stirred for 1 h at 0° C. under nitrogen atmosphere. The reaction was monitored by LCMS. The reaction was quenched by the addition of 1M sodium sulfite (50 mL) at 0° C. The aqueous layer was extracted with dichloromethane (5×50 mL). The resulting mixture was concentrated under reduced pressure. The residue was purified by silica gel column chromatography, eluted with petroleum ether/acetic ester (1:1) to afford CI-17 (1 g, 36.12%) as a yellow solid. LCMS (ESI, m/z): [M+H]⁺ 329.9.

### Intermediate 18, 1-{3-[4-({7-[1-(1-ethoxyethyl)pyrazol-4-yl}-8-isopropoxy-[1,2,4]triazolo[1,5-c]pyrimidin-2-yl]amino)-3-fluorobenzenesulfonyl]phenyl]azetidin-3-one (CI-18)

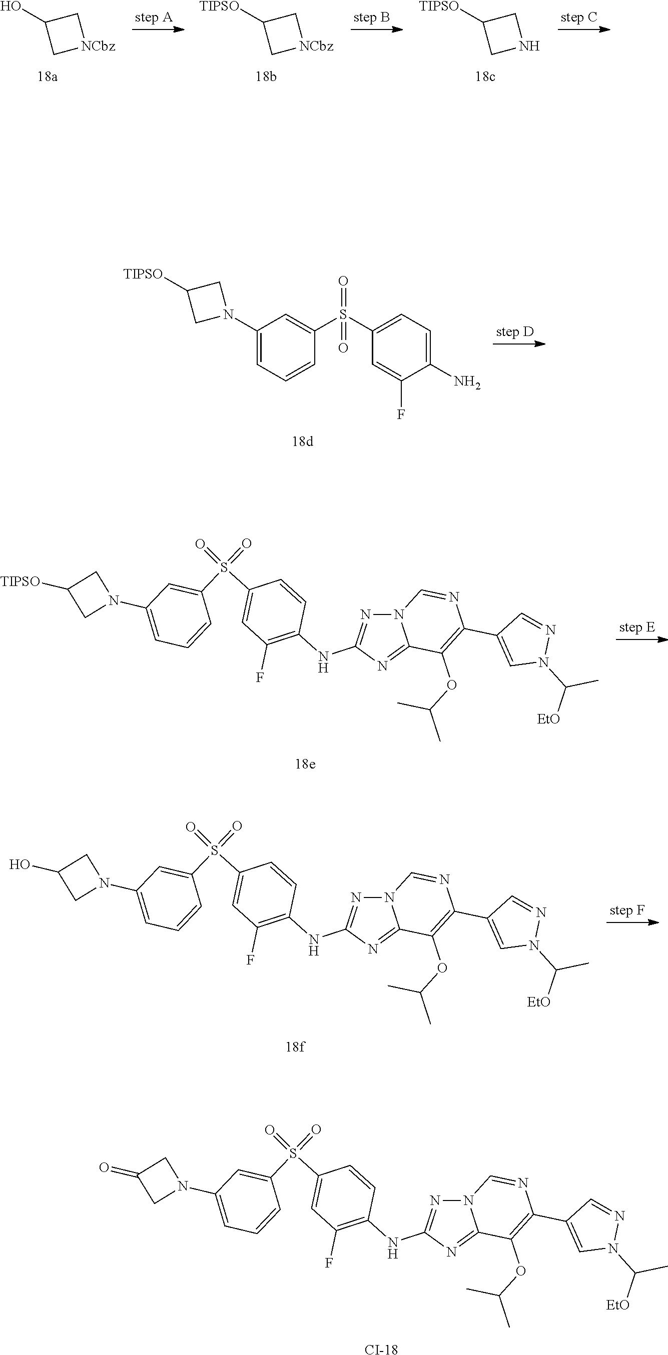

### Step A. benzyl 3-[(triisopropylsilyl)oxy]azetidine-1-carboxylate (18b)

To a stirred mixture of 18a (5 g, 24.128 mmol, 1 equiv) and Imidazole (4927.81 mg, 72.384 mmol, 3 equiv) in dichloromethane (10 mL) was added 4-N,N-dimethylaminopyridine (589.55 mg, 4.826 mmol, 0.2 equiv) and chlorotris(propan-2-yl) silane (9303.81 mg, 48.256 mmol, 2 equiv) at room temperature under nitrogen atmosphere. The resulting mixture was stirred for overnight at room temperature under nitrogen atmosphere. Desired product could be detected by LCMS. The resulting mixture was concentrated under reduced pressure. The residue was purified by silica gel column chromatography, eluted with petroleum ether/ethyl acetate(1:1) to afford 18b (7.5 g, 76.95%) as an off-white oil. LCMS (ESI, m/z): [M+H−100+41]⁺ 364.0.

### Step B. 3-[(triisopropylsilyl)oxy]azetidine (18c)

To a stirred mixture of 18b (7.5 g, 20.629 mmol, 1.00 equiv) in trifluoroethanol (20 mL) were added Pd/C (4.39 g, 41.258 mmol, 2 equiv) at room temperature under hydrogen atmosphere. The resulting mixture was stirred for overnight at room temperature under nitrogen atmosphere. Desired product could be detected by LCMS. The resulting mixture was filtered, the filter cake was washed with acetonitrile (30×10 mL). The filtrate was concentrated under reduced pressure. The resulting mixture was concentrated under reduced pressure. The crude product was used in the next step directly without further purification. LCMS (ESI, m/z): [M+H]⁺ 230.0.

### Step C. 2-fluoro-4-(3-{3-[(triisopropylsilyl)oxy]azetidin-1-yl}benzenesulfonyl)aniline (18d)

To a stirred solution of CI-17 (2 g, 6.057 mmol, 1 equiv) and 18c (2.78 g, 12.114 mmol, 2 equiv) in dioxane (20 mL) were added Pd₂(dba)₃ (1.11 g, 1.211 mmol, 0.2 equiv) and potassium phosphate tribasic (3.86 g, 18.171 mmol, 3 equiv) at room temperature under nitrogen atmosphere. The resulting mixture was stirred for overnight at 90° C. under nitrogen atmosphere. Desired product could be detected by LCMS. The resulting mixture was diluted with water (200 mL). The resulting mixture was extracted with ethyl acetate (600 mL). The combined organic layers were washed with brine (300 mL), dried over anhydrous sodium sulfate. After filtration, the filtrate was concentrated under reduced pressure. The residue was purified by silica gel column chromatography, eluted with dichloromethane/methanol (20:1) to afford 18d (1.3 g, 38.11%) as a Brown yellow solid. LCMS (ESI, m/z): [M+H]479.0.

### Step D. 7-[1-(1-ethoxyethyl) pyrazol-4-yl]-N-[2-fluoro-4-(3-{3-[(triisopropylsilyl)oxy]azetidin-1-yl}benzenesulfonyl)phenyl]-8-isopropoxy-[1,2,4]triazolo[1,5-c]pyrimidin-2-amine (18e)

To a stirred solution of 18d (1 g, 2.089 mmol, 1 equiv) and CI-1 (0.83 g, 2.110 mmol, 1.01 equiv) in dioxane (10 mL) were added Pd₂(dba)₃ (0.29 g, 0.313 mmol, 0.15 equiv), RuPhos (0.19 g, 0.418 mmol, 0.2 equiv) and cesium carbonate (2.04 g, 6.267 mmol, 3 equiv) at room temperature under nitrogen atmosphere. The resulting mixture was stirred for overnight at 90° C. under nitrogen atmosphere. Desired product could be detected by LCMS. The resulting mixture was diluted with water (300 mL). The resulting mixture was extracted with ethyl acetate (600 mL). The combined organic layers were washed with brine (20×10 mL), dried over anhydrous sodium sulfate. After filtration, the filtrate was concentrated under reduced pressure. The residue was purified by silica gel column chromatography, eluted with petroleum ether/ethyl acetate(1:1) to afford 18e (1.5 g, 81.49%) as a light yellow solid. LCMS (ESI, m/z): [M+H]⁺ 793.0.

### Step E. 1-{3-[4-({7-[1-(1-ethoxyethyl)pyrazol-4-yl]-8-isopropoxy-[1,2,4]triazolo[1,5-c]pyrimidin-2-yl}amino)-3-fluorobenzenesulfonyl]phenyl}azetidin-3-ol (18f)

To a stirred mixture of 18e (1.4 g, 1.765 mmol, 1 equiv) in tetrahydrofuran (8.75 mL, 107.983 mmol, 61.18 equiv) was added tetrabutylammonium fluoride (1.38 g, 5.295 mmol, 3 equiv) dropwise at 0° C. under nitrogen atmosphere. The resulting mixture was stirred for 1 h at 0° C. under nitrogen atmosphere. Desired product could be detected by LCMS.

The resulting mixture was diluted with water (200 mL). The resulting mixture was extracted with ethyl acetate (500 mL). The combined organic layers were washed with brine (200 mL), dried over anhydrous sodium sulfate. After filtration, the filtrate was concentrated under reduced pressure. The residue was purified by silica gel column chromatography, eluted with petroleum ether/ethyl acetate(1:1) to afford 18f (1.02 g, 81.67%) as a light yellow solid. LCMS (ESI, m/z): [M+H]637.0.

### Step F. 1-{3-[4-({7-[1-(1-ethoxyethyl)pyrazol-4-yl]-8-isopropoxy-[1,2,4]triazolo[1,5-c]pyrimidin-2-yl}amino)-3-fluorobenzenesulfonyl]phenyl}azetidin-3-one (CI-18)

To a stirred mixture of 18f (1 g, 1.571 mmol, 1 equiv) in dichloromethane (10 mL) was added Dess-Martin (1.33 g, 3.142 mmol, 2 equiv) at 0° C. under nitrogen atmosphere. The resulting mixture was stirred for overnight at 0° C. under nitrogen atmosphere. Desired product could be detected by LCMS. The resulting mixture was filtered, the filter cake was washed with dichloromethane (20×10 mL). The filtrate was concentrated under reduced pressure. The residue was purified by silica gel column chromatography, eluted with dichloromethane/methanol (20:1) to afford CI-18 (295 mg, 23.67%) as an off-white solid. LCMS (ESI, m/z): [M+H]⁺ 635.0.

### Intermediate 19. (3S)-3-[5-(3-{[3-(4-amino-3-fluorobenzenesulfonyl)phenyl]methyl]cyclobutyl)-3-methyl-2-oxo-1,3-benzodiazol-1-yl]-1-{[2-(trimethylsilyl)ethoxylmethyl]piperidine-2,6-dione (CI-19)

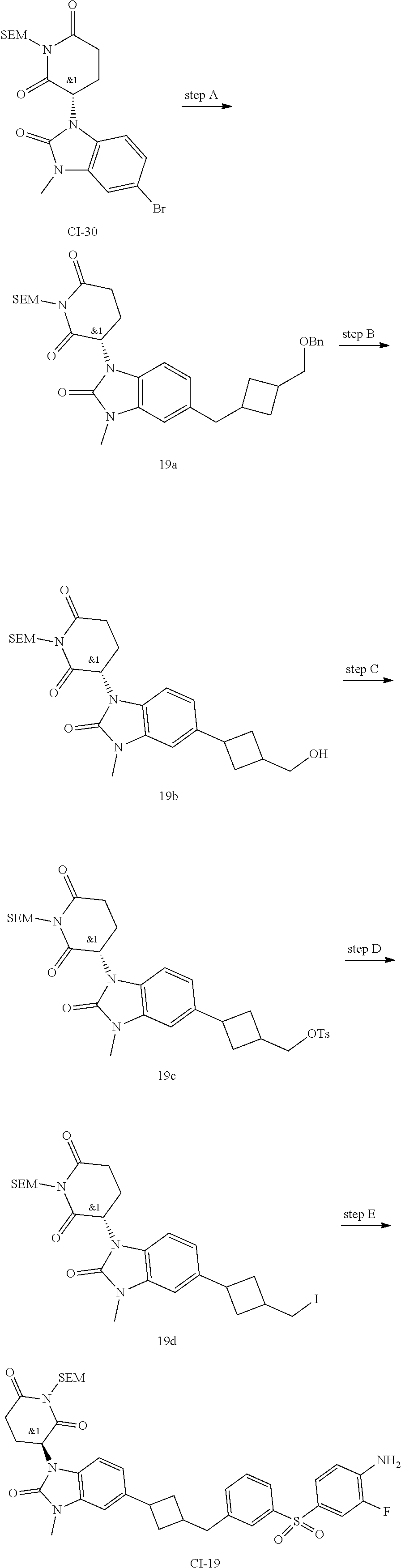

### Step A. (3S)-3-(5-{3-[(benzyloxy)methyl]cyclobutyl}-3-methyl-2-oxo-1,3-benzodiazol-1-yl)-1-{[2-(trimethylsilyl)ethoxy]methyl}piperidine-2,6-dione (19a)

A solution of CI-30 (700 mg, 1.494 mmol, 1 equiv),1,2-dibromoethane (112.29 mg, 0.598 mmol, 0.4 equiv) in tetrahydrofuran (1 mL) was treated with trimethylchlorosilane (64.94 mg, 0.598 mmol, 0.4 equiv) for 2 h at 50° C. under nitrogen atmosphere followed by the addition of Pd(dppf)C₁₋₂ (218.69 mg, 0.299 mmol, 0.2 equiv) dropwise/in portions at 60° C. Desired product could be detected by LCMS. The residue was purified by reversed-phase flash chromatography with the following conditions: column, C18 silica gel; mobile phase, acetonitrile in water (0.1% formic acid), 10% to 50% gradient in 10 min; detector, UV 254 nm. To afford 19a (400 mg, 47.48%) as a yellow solid. LCMS (ESI, m/z): [M+H]⁺ 578.30.

### Step B. (3S)-3-{5-[3-(hydroxymethyl)cyclobutyl]-3-methyl-2-oxo-1,3-benzodiazol-1-yl}-1-{[2-(trimethylsilyl)ethoxy]methyl}piperidine-2,6-dione (19b)

A solution of 19a (300 mg, 0.54 mmol, 1 equiv) and Pd/C (75 mg, 0.54 mmol, 1 equiv) in ethanol/tetrahydrofuran (0.5 mL) was stirred for 6 h at 50° C. under hydrogen atmosphere. Desired product could be detected by LCMS. The resulting mixture was filtered, the filter cake was washed with methanol (2×10 mL). The filtrate was concentrated under reduced pressure. The resulting mixture was used in the next step directly without further purification. LCMS (ESI, m/z): [M+H]⁺ 474.23.

### Step C. (3-{1-[(3S)-2,6-dioxo-1-{[2-(trimethylsilyl)ethoxy]methyl}piperidin-3-yl]-3-methyl-2-oxo-1,3-benzodiazol-5-yl}cyclobutyl)methyl 4-methylbenzenesulfonate (19c)

A solution of 19b (500 mg, 1.056 mmol, 1 equiv) and p-toluenesulfonyl chloride (241.50 mg, 1.267 mmol, 1.2 equiv) in dichloromethane (5 mL) was stirred for 1 h at room temperature under argon atmosphere. Desired product could be detected by LCMS. The residue was purified by silica gel column chromatography, eluted with petroleum ether/ethyl acetate(1:1) to afford 19c (600 mg, 90.53%) as a yellow solid. LCMS (ESI, m/z): [M+H]⁺ 628.24.

### Step D. (3S)-3-{5-[3-(iodomethyl)cyclobutyl]-3-methyl-2-oxo-1,3-benzodiazol-1-yl}-1-{[2-(trimethylsilyl) ethoxy]methyl}piperidine-2,6-dione (19d)

A solution of 19c (200 mg, 0.319 mmol, 1 equiv) and sodium iodide (143.25 mg, 0.957 mmol, 3 equiv) in 2-butanone (1 mL) was stirred for 1 h at 100° C. under argon atmosphere. Desired product could be detected by LCMS. The residue was purified by silica gel column chromatography, eluted with petroleum ether/ethyl acetate(1:1) to afford 19d (120 mg, 64.55%) as a yellow solid. LCMS (ESI, m/z): [M+H]⁺ 584.14.

### Step E. (3S)-3-[5-(3-{[3-(4-amino-3-fluorobenzenesulfonyl)phenyl]methyl}cyclobutyl)-3-methyl-2-oxo-1,3-benzodiazol-1-yl]-1-{[2-(trimethylsilyl)ethoxy]methyl}piperidine-2,6-dione (CI-19)

A solution of 19d (300 mg, 0.514 mmol, 1 equiv), Nickel(II) chloride ethylene glycol dimethyl ether complex (22.59 mg, 0.103 mmol, 0.2 equiv), sodium iodide (38.53 mg, 0.257 mmol, 0.5 equiv), Zn (134.45 mg, 2.056 mmol, 4 equiv) and CI-17 (9.57 mg, 0.103 mmol, 0.2 equiv) in N,N-dimethylacetamide (5 mL) was stirred for overnight at 60° C. under argon atmosphere. Desired product could be detected by LCMS. The residue was purified by silica gel column chromatography, eluted with petroleum ether/ethyl acetate(1:1) to afford CI-19 (100 mg, 27.52%) as a yellow solid. LCMS (ESI, m/z): [M+H]⁺ 707.27.

### Intermediate 20, 1-[3-(azetidin-3-yloxy)benzenesulfonyl]-N-{7-[1-(1-ethoxyethyl)pyrazol-4-yl}-8-isopropoxy-[1,2,41triazolo[1,5-cl pyrimidin-2-yl}piperidin-4-amine (CI-20)

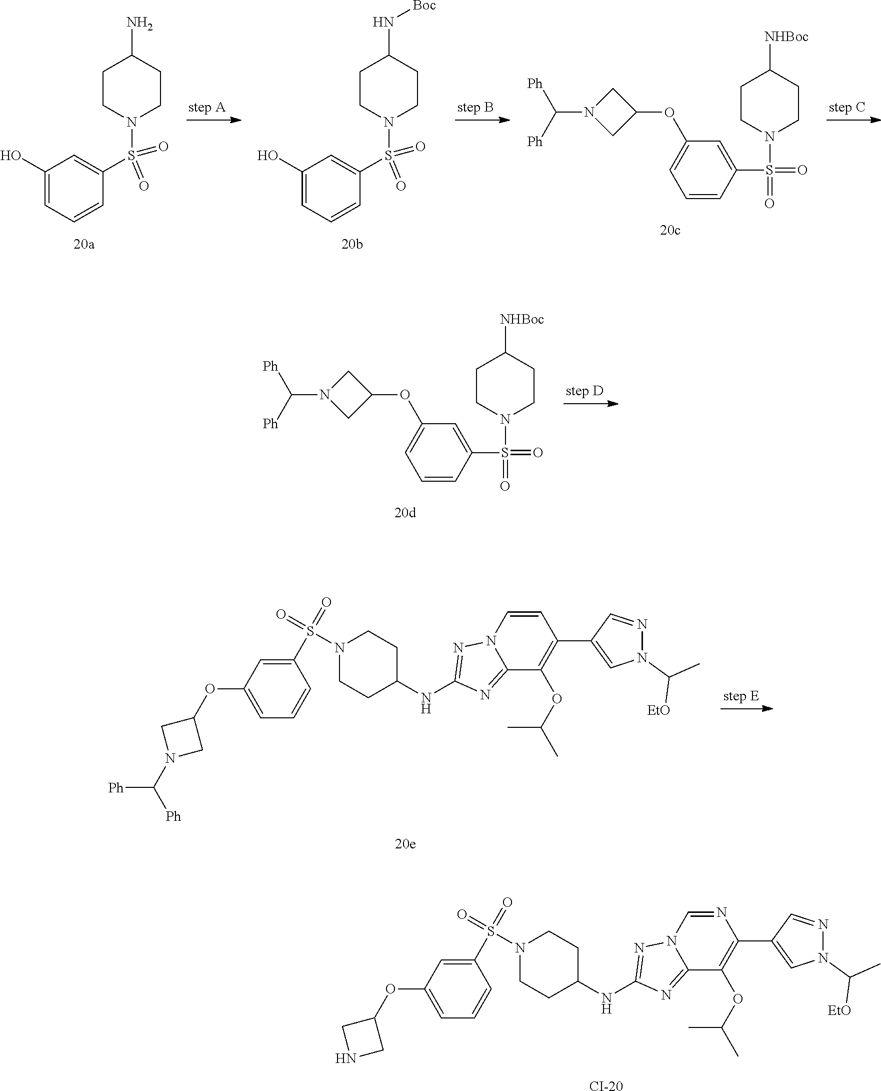

### Step A. tert-butyl N-[1-(3-hydroxybenzenesulfonyl)piperidin-4-yl]carbamate (20b)

To a stirred solution of 20a (1 g, 3.901 mmol, 1 equiv) and sodium bicarbonate (0.66 g, 7.802 mmol, 2 equiv) in water (10 mL) and tetrahydrofuran (10 mL) was added Boc₂O (0.85 g, 3.901 mmol, 1.0 equiv) dropwise at room temperature under nitrogen atmosphere. The resulting mixture was stirred for 1 h at room temperature under nitrogen atmosphere. Desired product could be detected by LCMS. The resulting mixture was concentrated under reduced pressure. The residue was purified by silica gel column chromatography, eluted with petroleum ether/ethyl acetate(1:1) to afford tert-butyl 20b (1 g, 71.91%) as a white solid. LCMS (ESI, m/z): [M+H]⁺ 357.4.

### Step B. tert-butyl N-[1-(3-{[1-(diphenylmethyl)azetidin-3-yl]oxy}benzenesulfonyl)piperidin-4-yl]carbamate (20c)

To a stirred solution of 20b (1 g, 2.806 mmol, 1 equiv) and 1-(diphenylmethyl)azetidin-3-ol (738.55 mg, 3.087 mmol, 1.1 equiv) in dimethyl sulfoxide (10 mL) was added cesium carbonate (2.74 g, 8.418 mmol, 3 equiv) in portions at room temperature under nitrogen atmosphere. The resulting mixture was stirred for 1 h at 90° C. under nitrogen atmosphere. Desired product could be detected by LCMS. The residue was purified by silica gel column chromatography, eluted with petroleum ether/ethyl acetate(5:1) to afford 20c (800 mg, 49.36%) as a white solid. LCMS (ESI, m/z): [M+H]⁺ 578.7.

### Step C. 1-(3-{[1-(diphenylmethyl)azetidin-3-yl]oxy}benzenesulfonyl)piperidin-4-amine (20d)

A solution of 20c (1 g, 1.731 mmol, 1 equiv) and HCl(gas)in 1,4-dioxane (2 mL, 65.826 mmol, 38.03 equiv) in acetonitrile (2 mL) was stirred for 1 h at 0° C. under nitrogen atmosphere. Desired product could be detected by LCMS. The resulting mixture was concentrated under reduced pressure. The residue was purified by reversed-phase flash chromatography with the following conditions: column, C18 silica gel; mobile phase, acetonitrile in water (10 mmol/L ammonium bicarbonate), 10% to 60% gradient in 10 min; detector, UV 254 nm. This resulted in 20d (700 mg, 84.67%) as a white solid. LCMS (ESI, m/z): [M+H]⁺ 478.6.

### Step D. 1-(3-{[1-(diphenylmethyl)azetidin-3-yl]oxy}benzenesulfonyl)-N-{7-[1-(1-ethoxyethyl)pyrazol-4-yl]-8-isopropoxy-[1,2,4]triazolo[1,5-c]pyrimidin-2-yl}piperidin-4-amine (20e)

To a stirred solution of 20d (400 mg, 0.837 mmol, 1 equiv), CI-1(252 mg, 0.837 mmol, 1 equiv) and RuPhos (78.16 mg, 0.167 mmol, 0.2 equiv) in 1,4-dioxane (4 ml) were added Pd₂(dba)₃ (153.38 mg, 0.167 mmol, 0.2 equiv) and sodium tert-butoxide (241.46 mg, 2.511 mmol, 3 equiv) in portions at room temperature under nitrogen atmosphere. Desired product could be detected by LCMS. The resulting mixture was stirred for 1 h at 100° C. under nitrogen atmosphere. The residue was purified by silica gel column chromatography, eluted with petroleum ether/ethyl acetate(1:1) to afford 20e (400 mg, 60.31%) as a yellow solid. LCMS (ESI, m/z): [M+H]⁺ 792.9.

### Step E. 1-[3-(azetidin-3-yloxy)benzenesulfonyl]-N-{7-[1-(1-ethoxyethyl)pyrazol-4-yl]-8-isopropoxy-[1,2,4]triazolo[1,5-c]pyrimidin-2-yl}piperidin-4-amine (CI-20)

To a solution of 20e (300 mg, 0.379 mmol, 1 equiv) acetic acid (1 mL, 17.452 mmol, 46.07 equiv) in 20 mL methanol were added Pd(OH)₂/C (10%, 150 mg) in a pressure tank. The mixture was hydrogenated at 50° C. under 50 psi of hydrogen pressure for 4 h, filtered through a Celite pad and concentrated under reduced pressure. Desired product could be detected by LCMS. The residue was purified by reversed-phase flash chromatography with the following conditions: column, C18 silica gel; mobile phase, acetonitrile in water (10 mmol/L ammonium bicarbonate), 10% to 60% gradient in 10 min; detector, UV 254 nm. This resulted in CI-20 (100 mg, 42.19%) as a white solid. LCMS (ESI, m/z): [M+H]⁺ 626.7.

### Intermediate 21, 3-(4-hydroxy-3-methyl-2-oxo-1,3-benzodiazol-1-yl)-1-{[2-(trimethylsilyl)ethoxylmethyl]piperidine-2,6-dione (CI-21)

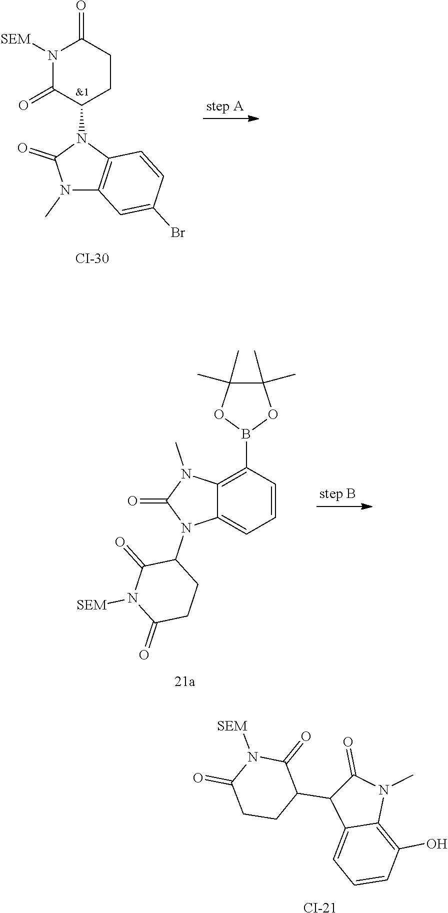

### Step A: 3-[3-methyl-2-oxo-4-(4,4,5,5-tetramethyl-1,3,2-dioxaborolan-2-yl)-1,3-benzodiazol-1-yl]-1-{[2-(trimethylsilyl)ethoxy]methyl}piperidine-2,6-dione (21a)

To a stirred mixture of CI-30 (400 mg, 0.854 mmol, 1 equiv) in tetrahydrofuran (4 mL) was added hydrogen peroxide (290.46 mg, 8.540 mmol, 10 equiv) in portions at 0° C. The resulting mixture was stirred for 2 h at room temperature. The reaction was monitored by TLC. The reaction was quenched with sat. sodium hyposulfite (aq.) at 0° C. The aqueous layer was extracted with ethyl acetate (3×30 mL). The resulting mixture was concentrated under reduced pressure. The residue was purified by silica gel column chromatography, eluted with petroleum ether/acetic ester (1:1) to afford 21a (250 mg, 56.79%) as a white solid. LCMS (ESI, m/z): [M+H]⁺ 516.2

### Step B: 3-(4-hydroxy-3-methyl-2-oxo-1,3-benzodiazol-1-yl)-1-{[2-(trimethylsilyl)ethoxy]methyl}piperidine-2,6-dione (CI-21)

To a stirred mixture of 21a (100 mg, 0.194 mmol, 1 equiv) in dioxane (2 mL) was added hydrogen peroxide (65.98 mg, 1.940 mmol, 10 equiv) in portions at 0° C. The resulting mixture was stirred for 2 h at room temperature. The reaction was monitored by LCMS. Desired product could be detected by LCMS. The reaction was quenched by the addition of saturated sodium bisulfite (aq.) (1 mL). The resulting mixture was extracted with ethyl acetate. The combined organic layers were dried over anhydrous sodium sulfate. After filtration, the filtrate was concentrated under reduced pressure to afford CI-21 (100 mg, crude) as a white solid. LCMS (ESI, m/z): [M+H]⁺ 356.2.

### Intermediate 22. 1-[3-(3-fluoro-4-{[8-isopropoxy-7-(1H-pyrazol-4-yl)-[1,2,41triazolo[1,5-c]pyrimidin-2-yl]amino}benzenesulfonyl)phenyl]piperidine-4-carbaldehyde (CI-22)

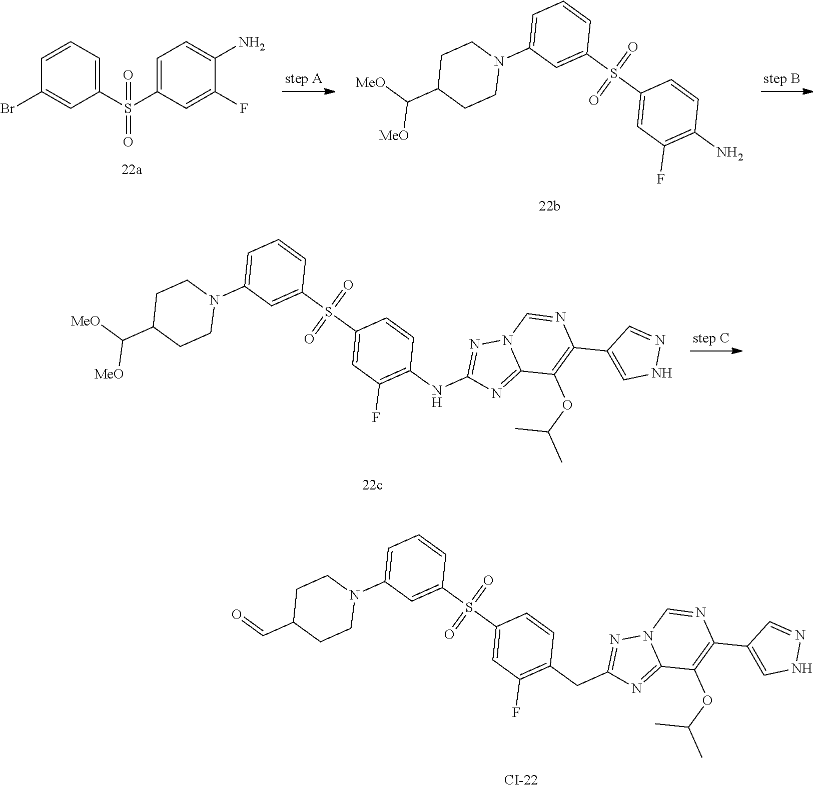 [SMILES: Nc1ccc(S(=O)(=O)c2cccc(Br)c2)cc1F ; COC(OC)C1CCN(c2cccc(S(=O)(=O)c3ccc(N)c(F)c3)c2)CC1 ; COC(OC)C1CCN(c2cccc(S(=O)(=O)c3ccc(Nc4nc5c(OC(C)C)c(-c6cn[nH]c6)ncn5n4)c(F)c3)c2)CC1 ; "22c" ; CC(C)Oc1c(-c2cn[nH]c2)ncn2nc(Cc3ccc(S(=O)(=O)c4cccc(N5CCC(C=O)CC5)c4)cc3F)nc12 ; "CI-22"]

### Step A. 4-{3-[4-(dimethoxymethyl)piperidin-1-yl]benzenesulfonyl}-2-fluoroaniline (22b)

To a stirred mixture of CI-17 (200 mg, 0.606 mmol, 1 equiv) and 4-(dimethoxymethyl) piperidine (1929.05 mg, 12.120 mmol, 20 equiv) in dioxane (5 mL) was added RuPhos (56.53 mg, 0.121 mmol, 0.2 equiv), potassium phosphate tribasic (385.74 mg, 1.818 mmol, 3 equiv) and RuPhos Palladacycle G3 (101.33 mg, 0.121 mmol, 0.2 equiv) at room temperature under nitrogen atmosphere. The resulting mixture was stirred for 2 h at 60° C. under nitrogen atmosphere. Desired product could be detected by LCMS. The resulting mixture was diluted with water (100 mL). The resulting mixture was extracted with ethyl acetate (2×100 mL). The combined organic layers were washed with brine (10×20 mL), dried over anhydrous sodium sulfate. After filtration, the filtrate was concentrated under reduced pressure. The residue was purified by silica gel column chromatography, eluted with petroleum ether/ethyl acetate(1:1) to afford 22b (146 mg, 53.10%) as a light yellow solid. LCMS (ESI, m/z): [M+H]⁺ 409.0.

### Step B. N-(4-{3-[4-(diethoxymethyl)piperidin-1-yl]benzenesulfonyl}-2-fluorophenyl)-7-[1-(1-ethoxyethyl)pyrazol-4-yl]-8-isopropoxy-[1,2,4]triazolo]1,5-c]pyrimidin-2-amine (22c)

To a stirred mixture of 22b (400 mg, 0.979 mmol, 1 equiv) and CI-1 (387.05 mg, 0.979 mmol, 1 equiv) in dioxane (5 mL) was added Pd₂(dba)₃ (179.34 mg, 0.196 mmol, 0.2 equiv), RuPhos (91.39 mg, 0.196 mmol, 0.2 equiv) and cesium carbonate (957.14 mg, 2.937 mmol, 3 equiv) at room temperature under nitrogen atmosphere. The resulting mixture was stirred for 2 h at 90° C. under nitrogen atmosphere. Desired product could be detected by LCMS. The resulting mixture was diluted with water (100 mL). The resulting mixture was extracted with ethyl acetate (200 mL). The combined organic layers were washed with brine (100 mL), dried over anhydrous sodium sulfate. After filtration, the filtrate was concentrated under reduced pressure. The residue was purified by silica gel column chromatography, eluted with petroleum ether/ethyl acetate(1:1) to afford 22c (205 mg, 23.70%) as a light yellow solid. LCMS (ESI, m/z): [M+H−100]⁺ 723.0.

### Step C. 1-[3-(3-fluoro-4-{[8-isopropoxy-7-(1H-pyrazol-4-yl)-[1,2,4]triazolo[1,5-c]pyrimidin-2-yl]amino}benzenesulfonyl)phenyl]piperidine-4-carbaldehyde (CI-22)

To a stirred mixture of 22c (100 mg, 0.138 mmol, 1 equiv) in dichloromethane (2 mL) was added trifluoroacetic acid (2 mL) dropwise at 0° C. under nitrogen atmosphere. The resulting mixture was stirred for 1 h at room temperature under hydrogen atmosphere. Desired product could be detected by LCMS. The resulting mixture was concentrated under reduced pressure. The crude product was used in the next step directly without further purification. This resulted in CI-22 (100 mg, crude) as an off-white solid. LCMS (ESI, m/z): [M+H]⁺ 605.0

### Intermediate 23. 3-[1-oxo-5-(4-oxopiperidin-1-yl)-3H-isoindol-2-yl]piperidine-2,6-dione (CI-23)

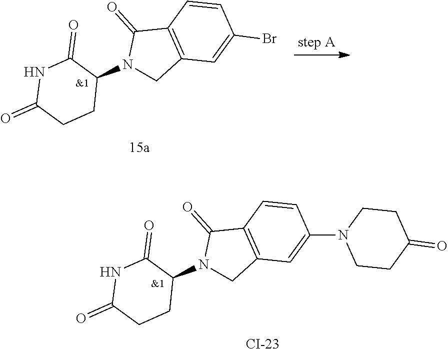 [SMILES: O=C1CC[C@H](N2Cc3cc(Br)ccc3C2=O)C(=O)N1 ; O=C1CCN(c2ccc3c(c2)CN([C@H]2CCC(=O)NC2=O)C3=O)CC1 ; "CI-23"]

### Step A: 3-[1-oxo-5-(4-oxopiperidin-1-yl)-3H-isoindol-2-yl]piperidine-2,6-dione (CI-23)

To a stirred mixture of 15a (100 mg, 0. 31 mmol, 1 equiv) and 4-piperidinone (36.8 mg, 0.37 mmol, 1.2 equiv) in N,N-dimethylformamide (5 mL) was added cesium carbonate (403.3 mg, 1.24 mmol, 4 equiv) and RuPhos Palladacycle Gen.3 (38.8 mg, 0.005 mmol, 0.15 equiv). The resulting mixture was stirred for 12 h at 80° C. under air atmosphere. Desired product could be detected by LCMS. The reaction was quenched by the addition of water (5 mL). The resulting mixture was extracted with ethyl acetate. The combined organic layers were dried over anhydrous sodium sulfate. After filtration, the filtrate was concentrated under reduced pressure. The residue was purified by silica gel column chromatography, eluted with petroleum ether/ethyl acetate (2:1) to afford 15b (100 mg, 94.43%) as a white solid. LCMS (ESI, m/z): [M+H]⁺ 342.1.

### Intermediate 24. N-{4-[3-(Azetidin-3-yloxy)benzenesulfonyl]-2-fluorophenyl]-8-isopropoxy-7-(1H-pyrazol-4-yl)-[1,2,4]triazolo[1,5-c]pyrimidin-2-amine (CI-24)

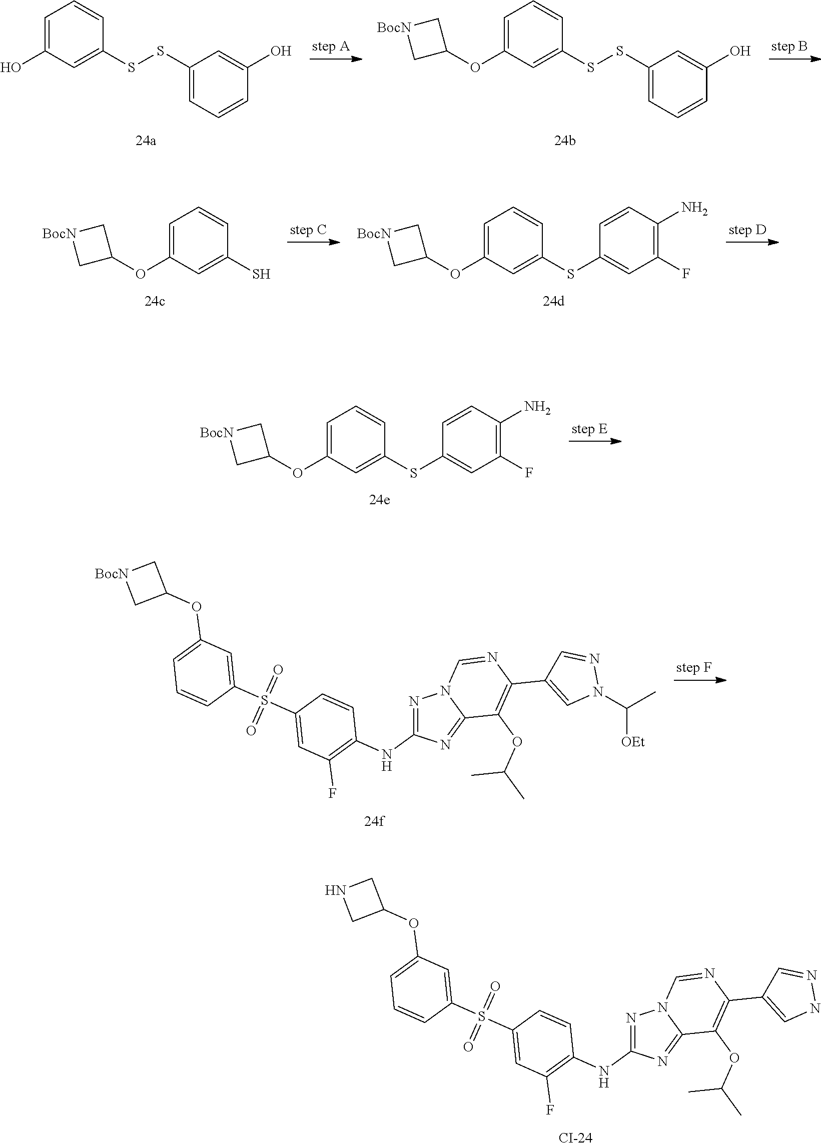

### Step A. Tert-butyl 3-[3-({[(3-{[1-(tert-butoxycarbonyl)azetidin-3-yl]oxy}phenyl)sulfanyl]methyl}sulfanyl)phenoxy]azetidine-1-carboxylate (24b)

To a stirred solution of 24a (2 g, 7.565 mmol, 1 equiv) and NaH (0.76 g, 18.913 mmol, 2.5 equiv, 60%) in N,N-dimethylformamide (10 mL) was added tert-butyl 3-iodoazetidine-1-carboxylate (5.57 g, 19.669 mmol, 2.6 equiv) at 0° C. The resulting mixture was stirred for overnight at 60° C. Desired product could be detected by LCMS. The resulting mixture was diluted with EA (100 mL). The resulting mixture was washed with 3×10 mL of water. The combined organic layers were dried over anhydrous sodium sulfate. After filtration, the filtrate was concentrated under reduced pressure. The residue was purified by silica gel column chromatography, eluted with petroleum ether/ethyl acetate(3:1) to afford 24b (1.5 g, 34.50%) as a yellow oil. LCMS (ESI, m/z): [M+H]⁺ 561.7

### Step B. Tert-butyl 3-(3-sulfanylphenoxy)azetidine-1-carboxylate (24c)

Conc.HCl (0.68 mL, 8.171 mmol, 9.16 equiv) was added to the solution of 24b (500 mg, 0.892 mmol, 1 equiv) in methanol (3.4 mL) and tetrahydrofuran (4.8 mL). Then Zn (5.83 g, 89.200 mmol, 100 equiv) was added slowly at 0° C. The resulting mixture was stirred for 1 h at 0° C. Desired product could be detected by LCMS. The mixture was filtered, and diluted with EA (100 mL). The organic layer were washed with saturated aqueous sodium bicarbonate (20 mL) and brine (20 mL), dried over anhydrous sodium sulfate, filtered and concentrated. The residue was purified by silica gel column chromatography, eluted with petroleum ether/ethyl acetate(3:1) to afford 24c (65 mg, 25.91%) as a colorless oil. LCMS (ESI, m/z): [M+H]⁺ 282.4

### Step C. Tert-butyl 3-{3-[(4-amino-3-fluorophenyl)sulfanyl]phenoxy}azetidine-1-carboxylate (24d)

A solution of 2-fluoro-4-iodoaniline (122 mg, 0.515 mmol, 1 equiv) and 24c (152.07 mg, 0.541 mmol, 1.05 equiv), Pd(PPh₃)₄(59.48 mg, 0.052 mmol, 0.1 equiv), sodium tert-butoxide (123.67 mg, 1.288 mmol, 2.5 equiv) in ethanol (4 mL) was stirred for 2 h at 90° C. under argon atmosphere. The resulting mixture was extracted with ethyl acetate and water. The combined organic layers were dried over anhydrous sodium sulfate. After filtration, the filtrate was concentrated under reduced pressure. The residue was purified by Prep-TLC (petroleum ether/ethyl acetate2:1) to afford 24d (130 mg, 64.68%) as a yellow oil. LCMS (ESI, m/z): [M+H]⁺ 391.5

### Step D. Tert-butyl 3-[3-(4-amino-3-fluorobenzenesulfonyl)phenoxy]azetidine-1-carboxylate (24e)

To a stirred solution of 24d (175 mg, 0.448 mmol, 1 equiv) in dichloromethane (3 mL) was added m-chloroperoxybenzoic acid (232.01 mg, 1.344 mmol, 3 equiv) dropwise at 0° C. The resulting mixture was stirred for 1 h at 0° C. Desired product could be detected by LCMS. The resulting mixture was extracted with dichloromethane and water. The combined organic layers were dried over anhydrous sodium sulfate. After filtration, the filtrate was concentrated under reduced pressure. The residue was purified by Prep-TLC (petroleum ether/ethyl acetate 1:1) to afford 24e (110 mg, 58.10%) as a yellow oil. LCMS (ESI, m/z): [M+H]⁺ 423.5

### Step E. Tert-butyl 3-{3-[4-({7-[1-(1-ethoxyethyl)pyrazol-4-yl]-8-isopropoxy-[1,2,4]triazolo[1,5-c]pyrimidin-2-yl}amino)-3-fluorobenzenesulfonyl]phenoxy}azetidine-1-carboxylate (24f)

To a solution of CI-1 (93 mg, 0.235 mmol, 1 equiv) and 24e (99.40 mg, 0.235 mmol, 1.00 equiv) in dioxane (2 mL) were added Pd₂(dba)₃ (21.55 mg, 0.024 mmol, 0.1 equiv) and RuPhos (10.98 mg, 0.024 mmol, 0.1 equiv), cesium carbonate (191.65 mg, 0.587 mmol, 2.5 equiv). The resulting mixture was stirred for 1 h at 90° C. under argon atmosphere. Desired product could be detected by LCMS. The resulting mixture was extracted with EtOEt and water. The combined organic layers were dried over anhydrous sodium sulfate. After filtration, the filtrate was concentrated under reduced pressure. The residue was purified by Prep-TLC (petroleum ether/ethyl acetate1:1) to afford 24f (140 mg, 80.75%) as a yellow oil. LCMS (ESI, m/z): [M+H]⁺ 737.8

### Step F. N-{4-[3-(Azetidin-3-yloxy)benzenesulfonyl]-2-fluorophenyl}-8-isopropoxy-7-(1H-pyrazol-4-yl)-[1,2,4]triazolo[1,5-c]pyrimidin-2-amine (CI-24)

To a stirred solution of 24f (120 mg, 0.163 mmol, 1 equiv) in acetonitrile (3 mL) was added 4M HCl(gas)in 1,4-dioxane (0.6 mL, 2.400 mmol, 14.74 equiv) dropwise at 0° C. The resulting mixture was stirred for 2 h at 0° C. The resulting mixture was concentrated under reduced pressure. The residue was purified by reversed-phase flash chromatography with the following conditions: column, C18 silica gel; mobile phase, acetonitrile in water (0.1% formic acid), 0% to 70% gradient in 30 min; detector, UV 254 nm. This resulted in CI-24 (60 mg, 65.25%) as a yellow solid. LCMS (ESI, m/z): [M+H]⁺ 565.6

### Intermediate 25. 1-(1-methyl-7-11-[3-(methylamino)propyl]piperidin-4-yl}indazol-3-yl)-1,3-diazinane-2,4-dione (CI-25)

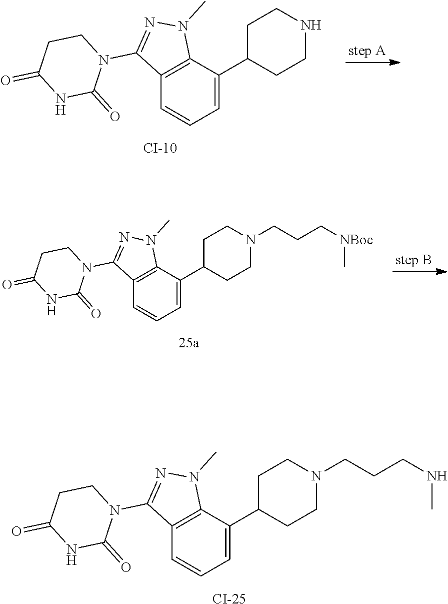

Step A. N-(3-{4-[3-(2,4-dioxo-1,3-diazinan-1-yl)-1-methylindazol-7-yl]piperidin-1-yl}propyl)-N-methylcarbamate (25a)

To a solution of CI-10 (85 mg, 0.260 mmol, 1 equiv) in N,N-dimethylformamide (1 mL) was added acetic acid (0.1 mL, 1.745 mmol, 6.72 equiv) and tert-butyl N-methyl-N-(3-oxopropyl)carbamate (48.61 mg, 0.260 mmol, 1 equiv) at room temperature. The mixture was stirred for 15 min. Add sodium triacetoxyborohydride (165.08 mg, 0.780 mmol, 3 equiv), tetraethyl titanate (592.25 mg, 2.600 mmol, 10 equiv) at room temperature to stir for 1 hour. Desired product could be detected by LCMS. The residue was purified by reversed-phase flash chromatography with the following conditions: column, C18 silica gel; mobile phase, water in acetonitrile, 20% to 70% gradient in 30 min; detector, UV 254 nm. The resulting mixture was concentrated under reduced pressure to afford 25a (60 mg, 46.35%) as white solid. LCMS (ESI, m/z): [M+H]⁺=499.3.

### Step B. 1-(1-methyl-7-{1-[3-(methylamino)propyl]piperidin-4-yl}indazol-3-yl)-1,3-diazinane-2,4-dione (CI-25)

To a solution of 25a (60 mg, 0.120 mmol, 1 equiv) in acetonitrile (1 mL, 19.024 mmol) was added HCl(gas)in 1,4-dioxane (0.2 mL, 6.583 mmol, 54.70 equiv) at 0° C. The mixture was stirred for 30 min. Desired product could be detected by LCMS. The resulting mixture was concentrated under reduced pressure to afford CI-25 (45 mg, 93.84%) as white solid. LCMS (ESI, m/z): [M+H]⁺ 399.3.

### Intermediate 26. 3-(3-methyl-4-((7-(7-((4-((7′-(2-methylcyclopentyl)-6′-oxo-6′,7′-dihydrospiro[cyclopropane-1,5′-pyrrolo[2,3-ylpyrimidin-2′-yl)amino)piperidin-1-yl)sulfonyl)-2,7-diazaspiro[4.41nonan-2-yl)heptyl)oxy)-2-oxo-2,3-dihydro-1H-benzo[d]imidazol-1-yl)-1-((2-(trimethylsilyl)ethoxy)methyl)piperidine-2,6-dione (CI-26)

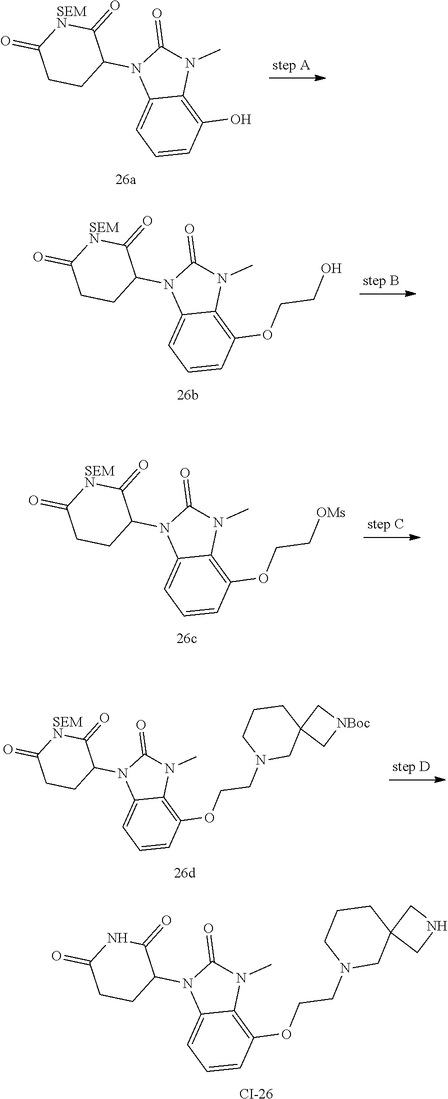

### Step A. 3-(4-(2-hydroxyethoxy)-3-methyl-2-oxo-2,3-dihydro-1H-benzo[d]imidazol-1-yl)-1-((2-(trimethylsilyl)ethoxy)methyl)piperidine-2,6-dione (26b)

A mixture of 26a (200 mg, 0.493 mmol, 1 equiv) and 2-bromoethanol (183.44 mg, 1.48 mmol, 3 equiv), potassium carbonate (195.31 mg, 1.48 mmol, 3 equiv), sodium iodide 5 (73.89 mg, 0.493 mmol, 1 equiv) in dimethyl sulfoxide (2 mL) was stirred for 16 h at 80° C. Desired product could be detected by LCMS. The aqueous layer was extracted with ethyl acetate (3×10 mL). The resulting mixture was concentrated under reduced pressure. The residue was purified by silica gel column chromatography, eluted with dichloromethane/methanol (30:1) to afford 26b (126 mg, 57.01% yield) as a white oil. LCMS (ESI, m/z): [M+H]⁺ 422.2

### Step B. 2-((1-(2,6-dioxo-1-((2-(trimethylsilyl)ethoxy)methyl)piperidin-3-yl)-3-methyl-2-oxo-2,3-dihydro-1H-benzo[d]imidazol-4-yl)oxy)ethyl methanesulfonate (26c)

A solution of 26b (100 mg, 0.222 mmol, 1 equiv) in dichloromethane (10.00 mL) was treated with triethylamine (45.02 mg, 0.444 mmol, 2 equiv) for 1 min at 0° C. under nitrogen atmosphere followed by the addition of MsCl (38.22 mg, 0.333 mmol, 1.5 equiv) dropwise at 0° C. The resulting mixture was stirred for 1 h at 25° C. under nitrogen atmosphere. Desired product could be detected by LCMS. The aqueous layer was extracted with ethyl acetate (3×10 mL). The resulting mixture was concentrated under reduced pressure. to afford 26c (100 mg, 85.20% yield) as a white oil. LCMS (ESI, m/z): [M+H]⁺ 528.1

### Step C. tert-butyl 6-(2-((1-(2,6-dioxo-1-((2-(trimethylsilyl)ethoxy)methyl)piperidin-3-yl)-3-methyl-2-oxo-2,3-dihydro-1H-benzo[d]imidazol-4-yl)oxy)ethyl)-2,6-diazaspiro[3.5]nonane-2-carboxylate (26d)

A solution of 26c (110 mg, 0.208 mmol, 1 equiv), tert-butyl 2,6-diazaspiro[3.5]nonane-2-carboxylate (94.36 mg, 0.416 mmol, 2 equiv) in acetonitrile (5 mL) was treated with potassium carbonate (86.43 mg, 0.624 mmol, 3 equiv) at 25° C. under nitrogen atmosphere followed by the addition of potassium iodide (34.61 mg, 0.208 mmol, 1 equiv) in portions at 25° C. The resulting mixture was stirred for 16 h at 80° C. under nitrogen atmosphere. Desired product could be detected by LCMS. The residue was purified by Prep-TLC (ethyl acetate) to afford 26d (100 mg, 72.91% yield) as a white solid. LCMS (ESI, m/z): [M+H]⁺ 658.3

### Step D. 3-(3-methyl-4-((7-(7-((4-((7′-(2-methylcyclopentyl)-6′-oxo-6′,7′-dihydrospiro[cyclopropane-1,5′-pyrrolo[2,3-d]pyrimidin]-2′-yl)amino)piperidin-1-yl)sulfonyl)-2,7-diazaspiro[4.4]nonan-2-yl) heptyl)oxy)-2-oxo-2,3-dihydro-1H-benzo[d]imidazol-1-yl)-1-((2-(trimethylsilyl)ethoxy)methyl) piperidine-2,6-dione (CI-26)

A solution of 27d (100 mg, 0.150 mmol, 1 equiv) in dichloromethane (3 mL) was treated with trifluoroacetic acid (519.95 mg, 4.500 mmol, 30 equiv) for 1 min at 0° C. under nitrogen atmosphere. The resulting mixture was stirred for 1 h at 25° C. under nitrogen atmosphere. To the above mixture was added tetramethylethylenediamine (16.12 mg, 0.180 mmol, 1.2 equiv), triethylamine (922.96 mg, 9.010 mmol, 60 equiv) dropwise over 1 min at 0° C. The resulting mixture was stirred for additional 1 h at 25° C. Desired product could be detected by LCMS. The solvent was removed under reduced pressure. This resulted in CI-26 (80 mg) as a crude product which was used in the next step directly. LCMS (ESI, m/z): [M+H]⁺ 428.2.

### Intermediate 27. 3-(5-bromo-1-oxoisoindolin-2-yl)-1-((2-(trimethylsilyl)ethoxy)methyl)piperidine-2,6-dione (CI-27)

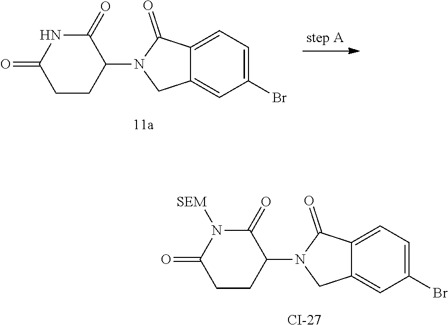

### Step A: 3-(5-bromo-1-oxoisoindolin-2-yl)-1-((2-(trimethylsilyl)ethoxy)methyl)piperidine-2,6-dione (CI-27)

To a stirred mixture of 11a (100 mg, 0.218 mmol, 1 equiv) and N,N-diisopropylethylamine (90.41 mg, 0.654 mmol, 4 equiv) in N,N-dimethylformamide (2.5 mL) was added (2-(chloromethoxy)ethyl)trimethylsilane (30.95 mg, 0.218 mmol, 3 equiv) in portions at 0° C. The resulting mixture was stirred for 16 h at room temperature. Desired product could be detected by LCMS. The reaction was quenched by the addition of water (10 mL). The resulting mixture was extracted with dichloromethane. The combined organic layers were dried over anhydrous sodium sulfate. After filtration, the filtrate was concentrated under reduced pressure. The residue was purified by silica gel column chromatography, eluted with petroleum ether/acetic ester (10:1) to afford CI-30 (100 mg, 71.23%) as a white solid. LCMS (ES, m/z): [M+H]⁺ 453.1.

### Intermediate 28. 1-{3-[4-(17-[1-(1-ethoxyethyl)pyrazol-4-yl]-8-isopropoxy-[1,2,4]triazolo[1,5-c]pyrimidin-2-yl}amino)-3-fluorobenzenesulfonyl]phenyl]azetidin-3-one (CI-28)

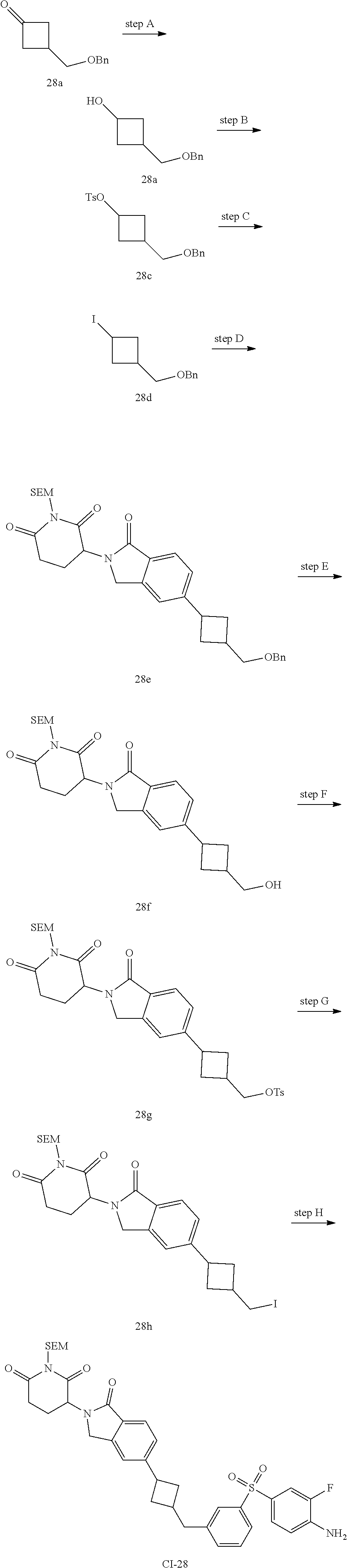

### Step A. 3-((benzyloxy)methyl)cyclobutan-1-ol (28b)

To a stirred solution of 28a (24.5 g, 128.783 mmol, 1 equiv) in ethanol (200 mL) was added sodium borohydride (7.31 g, 193.174 mmol, 1.5 equiv) in portions at 0° C. The resulting mixture was stirred for 5 h at room temperature under nitrogen atmosphere. Desired product could be detected by LCMS. The reaction was quenched slowly by the addition of saturated ammonium chloride (aq.) (200 mL) at 0° C. The resulting mixture was extracted with ethyl acetate (2×300 mL). The combined organic layers were washed with brine (2×200 mL), dried over anhydrous sodium sulfate. After filtration, the filtrate was concentrated under reduced pressure. This resulted in 28b (24.5 g, 98.95%) as a colorless oil. LCMS (ESI, m/z): [M+H]⁺ 193.1.

### Step B. 3-((benzyloxy)methyl)cyclobutyl 4-methylbenzenesulfonate (28c)

To a stirred solution of 28b (24.5 g, 127.433 mmol, 1 equiv), Dabco (0.71 g, 6.372 mmol, 0.05 equiv) and triethylamine (38.69 g, 382.299 mmol, 3 equiv) in dichloromethane (250 mL) was added p-toluenesulfonyl chloride (26.72 g, 140.176 mmol, 1.1 equiv)in portions at 0° C. under nitrogen atmosphere. The resulting mixture was stirred for 2 h at 0° C. to room temperature under nitrogen atmosphere. The reaction was monitored by LCMS. Desired product could be detected by LCMS. The resulting mixture was washed with 2×200 mL of saturated ammonium chloride (a.q.). The combined organic layers were washed with brine, dried over anhydrous sodium sulfate. After filtration, the filtrate was concentrated under reduced pressure. This resulted in 28c (44 g, 99.67%) as an orange oil. LCMS (ESI, m/z): [M+H]⁺ 347.1.

### Step C. (((3-iodocyclobutyl) methoxy)methyl)benzene (28d)

To a stirred solution of 28c (45 g, 129.893 mmol, 1 equiv) in acetone (400 mL) were added iodosodium (97.35 g, 649.465 mmol, 5 equiv) under nitrogen atmosphere. The resulting mixture was stirred for 12 hours at 100° C. under nitrogen atmosphere. The reaction was monitored by LCMS. Desired product could be detected by LCMS. The resulting mixture was diluted with saturated aqueous ammonium chloride (20 mL). The resulting mixture was extracted with ethyl acetate (2×15 mL). The combined organic layers were washed with brine (2×15 mL), dried over anhydrous sodium sulfate. After filtration, the filtrate was concentrated under reduced pressure. The residue was purified by silica gel column chromatography, eluted with 0% to 85% ethyl acetate in PE. Pure fractions were evaporated to dryness to afford 28d (35 g, 89.18%) as a white solid. LCMS (ESI, m/z): [M+H]⁺ 303.

### Step D. 3-(5-(3-((benzyloxy)methyl)cyclobutyl)-1-oxoisoindolin-2-yl)-1-((2-(trimethylsilyl) ethoxy)methyl)piperidine-2,6-dione (28e)

To a stirred solution of 28d (1 g, 3.310 mmol, 1 equiv) in N,N-dimethylacetamide (10 mL) were added CI-27 (2250.87 mg, 4.965 mmol, 1.5 equiv), 1H-imidazole-4-carbonitrile (61.62 mg, 0.662 mmol, 0.2 equiv), sodium iodide (248.04 mg, 1.655 mmol, 0.5 equiv), Zinc powder (865.52 mg, 13.240 mmol, 4 equiv), nickel chloride (85.78 mg, 0.662 mmol, 0.2 equiv) and trifluoroacetic acid (75.47 mg, 0.662 mmol, 0.2 equiv) under nitrogen atmosphere. The resulting mixture was stirred for 15 hours at 50° C. under nitrogen atmosphere. The reaction was monitored by LCMS. Desired product could be detected by LCMS. The mixture was allowed to cool down to room temperature. The resulting mixture was diluted with saturated aqueous ammonium chloride (20 mL). The resulting mixture was extracted with ethyl acetate (2×15 mL). The combined organic layers were washed with brine (2×15 mL), dried over anhydrous sodium sulfate. After filtration, the filtrate was concentrated under reduced pressure. The residue was purified by silica gel column chromatography, eluted with 0% to 15% ethyl acetate in PE. Pure fractions were evaporated to dryness to afford 28e (500 mg, 24.78%) as a white solid. LCMS (ESI, m/z): [M+H]⁺ 549.2

### Step E. 3-(5-(3-(hydroxymethyl)cyclobutyl)-1-oxoisoindolin-2-yl)-1-((2-(trimethylsilyl)ethoxy)methyl) piperidine-2,6-dione (28f)

To a stirred solution of 28e (4 g, 7.289 mmol, 1 equiv) in ethanol (50 mL) were added Pd/C (3878.60 mg, 36.445 mmol, 5 equiv). The resulting mixture was stirred for 3 hours at 60° C. under hydrogen atmosphere. The reaction was monitored by LCMS. Desired product could be detected by LCMS. The mixture was filtered through a Celite pad and concentrated under reduced pressure to get 3 g crude product and the residue was used for next step directly. LCMS (ESI, m/z): [M+H]⁺ 459.2

### Step F. {3-[2-(2,6-dioxo-1-{[2-(trimethylsilyl)ethoxy]methyl}piperidin-3-yl)-1-oxo-3H-isoindol-5-yl]cyclobutyl}methyl 4-methylbenzenesulfonate (28g)

To a stirred solution of 28f (4.5 g, 9.812 mmol, 1 equiv) in dichloromethane (50 mL) were added p-toluenesulfonyl chloride (3741.05 mg, 19.624 mmol, 2 equiv) and trimethylamine (2978.67 mg, 29.436 mmol, 3 equiv). The resulting mixture was stirred for 3 hours at 25° C. under hydrogen atmosphere. The reaction was monitored by LCMS. Desired product could be detected by LCMS. The resulting mixture was diluted with saturated aqueous ammonium chloride (20 mL). The resulting mixture was extracted with ethyl acetate (2×15 mL). The combined organic layers were washed with brine (2×15 mL), dried over anhydrous sodium sulfate. After filtration, the filtrate was concentrated under reduced pressure. The residue was purified by silica gel column chromatography, eluted with 0% to 15% ethyl acetate in PE. Pure fractions were evaporated to dryness to afford 28g (3 g, 47.40%) as a yellow oil. LCMS (ESI, m/z): [M+H]⁺ 613.3

### Step G. 3-(5-(3-(iodomethyl)cyclobutyl)-1-oxoisoindolin-2-yl)-1-((2-(trimethylsilyl)ethoxy)methyl) piperidine-2,6-dione (28h)

To a stirred solution of 28g (500 mg, 0.816 mmol, 1 equiv) in 2-butanone (1 mL) were added sodium iodide (489.20 mg, 3.264 mmol, 4 equiv) under nitrogen atmosphere. The resulting mixture was stirred for 1 hours at 100° C. under nitrogen atmosphere. The reaction was monitored by LCMS. Desired product could be detected by LCMS. The resulting mixture was diluted with saturated aqueous ammonium chloride (20 mL). The resulting mixture was extracted with ethyl acetate (2×15 mL). The combined organic layers were washed with brine (2×15 mL), dried over anhydrous sodium sulfate. After filtration, the filtrate was concentrated under reduced pressure. The residue was purified by silica gel column chromatography, eluted with 0% to 95% ethyl acetate in PE. Pure fractions were evaporated to dryness to afford 28h (300 mg, 58.21%) as a white solid. LCMS (ESI, m/z): [M+H]⁺ 569.1

### Step H: 3-(5-(3-(3-((4-amino-3-fluorophenyl)sulfonyl)benzyl)cyclobutyl)-1-oxoisoindolin-2-yl)-1-((2-(trimethylsilyl)ethoxy)methyl)piperidine-2,6-dione (CI-28)

To a stirred solution of 28h (200 mg, 0.352 mmol, 1 equiv) in N,N-dimethylacetamide (3 mL) was added CI-17 (231 mg, 0.704 mmol, 2 equiv), Nickel(II) acetylacetonate (27.00 mg, 0.106 mmol, 0.3 equiv),1H-imidazole-4-carbonitrile (10 mg, 0.106 mmol, 0.3 equiv), sodium iodide(52.80 mg, 0.352 mmol, 1 equiv), Zn(91.52 mg, 1.408 mmol, 4 equiv) and trifluoroacetic acid(31.68 mg, 0.352 mmol, 1 equiv). The resulting mixture was stirred for 12 h at 25 temperature under nitrogen atmosphere. Desired product could be detected by LCMS. The resulting mixture was diluted with saturated aqueous ammonium chloride (20 mL). The resulting mixture was extracted with ethyl acetate (2×15 mL). The combined organic layers were washed with brine (2×15 mL), dried over anhydrous sodium sulfate. After filtration, the filtrate was concentrated under reduced pressure. The residue was purified by silica gel column chromatography, eluted with 0% to 50% ethyl acetate in PE. Pure fractions were evaporated to dryness to afford CI-28 (90 mg, 36.9%) as a white solid. LCMS (ESI, m/z): [M+H]⁺ 692.2

### Intermediate 29. (3S)-3-[3-methyl-2-oxo-5-(piperidin-4-yl)-1,3-benzodiazol-1-yl]piperidine-2,6-dione (CI-29)

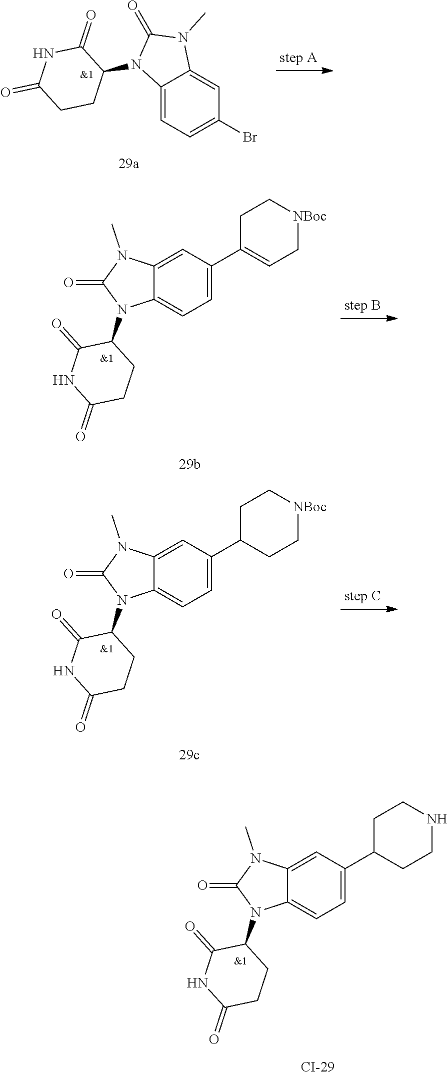 [SMILES: Cn1c(=O)n([C@H]2CCC(=O)NC2=O)c2ccc(Br)cc21 ; Cn1c(=O)n([C@H]2CCC(=O)NC2=O)c2ccc(C3=CCN(C(=O)OC(C)(C)C)CC3)cc21 ; Cn1c(=O)n([C@H]2CCC(=O)NC2=O)c2ccc(C3CCN(C(=O)OC(C)(C)C)CC3)cc21 ; Cn1c(=O)n([C@H]2CCC(=O)NC2=O)c2ccc(C3CCNCC3)cc21 ; "CI-29"]

### Step A. tert-butyl 4-{1-[(3S)-2,6-dioxopiperidin-3-yl]-3-methyl-2-oxo-1,3-benzodiazol-5-yl}-3,6-dihydro-2H-pyridine-1-carboxylate (29b)

To a stirred solution of 29a (500 mg, 1.479 mmol, 1 equiv) and tert-butyl 4-boranyl-3,6-dihydro-2H-pyridine-1-carboxylate (374.96 mg, 1.923 mmol, 1.3 equiv) in water (1 mL) and dioxane (5 mL) was added potassium phosphate tribasic (941.56 mg, 4.437 mmol, 3 equiv) and XPhos Pd G3 (187.73 mg, 0.222 mmol, 0.15 equiv) at room temperature under nitrogen atmosphere. The resulting mixture was stirred for 2 h at 80° C. under nitrogen atmosphere. Desired product could be detected by LCMS. The resulting mixture was diluted with water (200 mL). The resulting mixture was extracted with ethyl acetate (400 mL). The combined organic layers were washed with brine (10×10 mL), dried over anhydrous sodium sulfate. After filtration, the filtrate was concentrated under reduced pressure. The residue was purified by silica gel column chromatography, eluted with petroleum ether/ethyl acetate(1:1) to afford 29b (367 mg, 51.84%) as a white solid. LCMS (ESI, m/z): [M+H]⁺ 441.0.

### Step B. tert-butyl 4-{1-[(3S)-2,6-dioxopiperidin-3-yl]-3-methyl-2-oxo-1,3-benzodiazol-5-yl}piperidine-1-carboxylate (29c)

To a solution of 29b (360 mg, 0.817 mmol, 1 equiv) in tetrahydrofuran (5 mL) was added Pd/C (173.94 mg, 1.634 mmol, 2 equiv) under nitrogen atmosphere in a 10 mL round-bottom flask. The mixture was hydrogenated at room temperature for overnight under hydrogen atmosphere using a hydrogen balloon, filtered through a Celite pad and concentrated under reduced pressure. Desired product could be detected by LCMS. The residue was purified by silica gel column chromatography, eluted with dichloromethane/methanol (10:1) to afford 29c (347 mg, 76.76%) as an off-white solid. LCMS (ESI, m/z): [M+H−100]443.0.

### Step C. (3S)-3-[3-methyl-2-oxo-5-(piperidin-4-yl)-1,3-benzodiazol-1-yl]piperidine-2,6-dione (CI-29)

To a stirred solution of 29c (100 mg, 0.226 mmol, 1 equiv) in acetonitrile (4 mL) was added HCl (gas) in 1,4-dioxane (1 mL) dropwise at room temperature under nitrogen atmosphere. The resulting mixture was stirred for 1 h at 0° C. under nitrogen atmosphere. Desired product could be detected by LCMS. The resulting mixture was concentrated under reduced pressure. The crude product was used in the next step directly without further purification. This resulted in CI-29 (120 mg, crude) as an off-white solid. LCMS (ESI, m/z): [M+H]⁺ 343.0.

### Intermediate 30. rac-(R)-3-(5-bromo-3-methyl-2-oxo-2,3-dihydro-1H-benzo[d]imidazol-1-yl)-1-((2-(trimethylsilyl) ethoxy)methyl)piperidine-2,6-dione (CI-30)

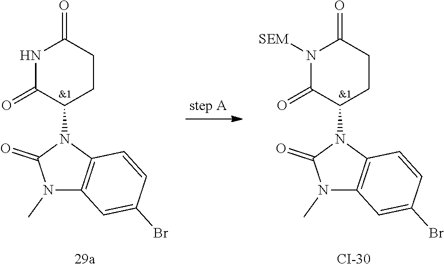 [SMILES: Cn1c(=O)n([C@H]2CCC(=O)NC2=O)c2ccc(Br)cc21 ; Cn1c(=O)n([C@H]2CCC(=O)N(COCC[Si](C)(C)C)C2=O)c2ccc(Br)cc21 ; "CI-30"]

### Step A: rac-(R)-3-(5-bromo-3-methyl-2-oxo-2,3-dihydro-1H-benzo[d]imidazol-1-yl)-1-((2-(trimethylsilyl)ethoxy)methyl)piperidine-2,6-dione (CI-30)

To a stirred mixture of 29a (100 mg, 0.218 mmol, 1 equiv) and N,N-diisopropylethylamine (90.41 mg, 0.654 mmol, 4 equiv) in N,N-dimethylformamide (2.5 mL) was added (2-(chloromethoxy)ethyl)trimethylsilane (30.95 mg, 0.218 mmol, 3 equiv) in portions at 0° C. The resulting mixture was stirred for 16 h at room temperature. Desired product could be detected by LCMS. The reaction was quenched by the addition of water (10 mL). The resulting mixture was extracted with dichloromethane. The combined organic layers were dried over anhydrous sodium sulfate. After filtration, the filtrate was concentrated under reduced pressure. The residue was purified by silica gel column chromatography, eluted with petroleum ether/acetic ester (10:1) to afford CI-30 (100 mg, 72.16%) as a white solid. LCMS (ES, m/z): [M+H]⁺ 468.1.

### Intermediate 31. 4-[3-(13-[4-(17-[1-(1-ethoxyethyl)pyrazol-4-yl]-8-isopropoxy-[1,2,4]triazolo[1,5-c]pyrimidin-2-yl]amino)-3-fluorobenzenesulfonyl]methyl]methyl)cyclobutyl]benzoic acid (CI-31)

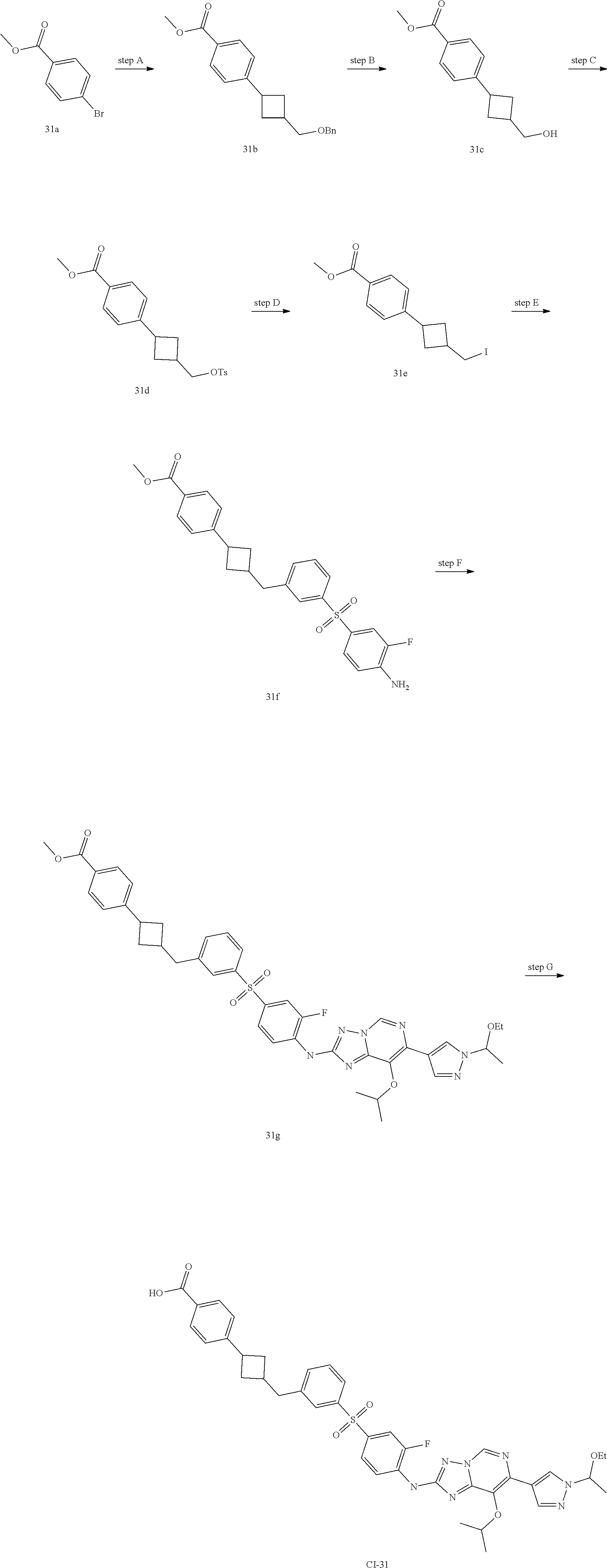

### Step A.4-{3-[(benzyloxy)methyl]cyclobutyl}benzoate (31b)

To a stirred solution of 31a (1.5 g, 6.975 mmol, 1 equiv) and {[(3-iodocyclobutyl)methoxy]methyl}benzene (4.22 g, 13.950 mmol, 2 equiv) in N,N-dimethylacetamide (20 mL) were added 4H-imidazole-4-carbonitrile (129.86 mg, 1.395 mmol, 0.2 equiv) and trifluoroacetic acid (318.14 mg, 2.790 mmol, 0.4 equiv) in portions at room temperature under nitrogen atmosphere. The resulting mixture was stirred for 6 h at room temperature under nitrogen atmosphere. Desired product could be detected by LCMS. The resulting mixture was extracted with ethyl acetate (3×40 mL). The combined organic layers were washed with brine (3×40 mL), dried over anhydrous sodium sulfate. After filtration, the filtrate was concentrated under reduced pressure. The residue was purified by silica gel column chromatography, eluted with petroleum ether/ethyl acetate(9:1) to afford 31b (1.2 g, 55.43%) as a light yellow oil. LCMS (ESI, m/z): [M+H]⁺ 311.1.

### Step B. methyl 4-[3-(hydroxymethyl)cyclobutyl]benzoate (31c)

To a solution of methyl 31b (1.2 g, 3.866 mmol, 1 equiv) in 3 mL tetrahydrofuran was added Pd(OH)₂/C (10%, 15 mg) under nitrogen atmosphere in a 25 mL round-bottom flask. The mixture was hydrogenated at room temperature for overnight under hydrogen atmosphere using a hydrogen balloon, filtered through a Celite pad and concentrated under reduced pressure. Desired product could be detected by LCMS. This resulted in 31c (800 mg, 93.94%) as a white solid. The crude product was used in the next step directly without further purification.

### Step C. methyl 4-(3-{[(4-methylbenzenesulfonyl)oxy]methyl}cyclobutyl)benzoate (31d)

To a stirred solution of 31c (770 mg, 3.496 mmol, 1 equiv) and p-toluenesulfonyl chloride (1.33 g, 6.992 mmol, 2 equiv) in dichloromethane (10 mL) were added trimethylamine (1.06 g, 10.488 mmol, 3 equiv) and triethylenediamine (39.15 mg, 0.350 mmol, 0.1 equiv) in portions at room temperature under nitrogen atmosphere. The resulting mixture was stirred for 2 h at room temperature under nitrogen atmosphere. Desired product could be detected by LCMS. The resulting mixture was extracted with ethyl acetate (3×20 mL). The combined organic layers were washed with brine (3×30 mL), dried over anhydrous sodium sulfate. After filtration, the filtrate was concentrated under reduced pressure. The residue was purified by silica gel column chromatography, eluted with petroleum ether/ethyl acetate(9:1) to afford 31d (1.2 g, 91.67%) as a white solid. LCMS (ESI, m/z): [M+H]303.

### Step D. methyl 4-[3-(iodomethyl)cyclobutyl]benzoate (31e)

To a stirred solution of 31d (1.2 g, 3.205 mmol, 1 equiv) in acetone (12 mL) was added sodium iodide (1441.10 mg, 9.615 mmol, 3 equiv) in portions at room temperature under nitrogen atmosphere. The resulting mixture was stirred for 1 h at 100° C. under nitrogen atmosphere. Desired product could be detected by LCMS. The resulting mixture was extracted with ethyl acetate (3×30 mL). The combined organic layers were washed with brine (3×30 mL), dried over anhydrous sodium sulfate. After filtration, the filtrate was concentrated under reduced pressure. The residue was purified by silica gel 30 column chromatography, eluted with petroleum ether/ethyl acetate(12:1) to afford 31e (920 mg, 86.95%) as a white solid. LCMS (ESI, m/z): [M+H]⁺ 331.0.

### Step E. methyl 4-(3-{[3-(4-amino-3-fluorobenzenesulfonyl)phenyl]methyl}cyclobutyl)benzoate (31f)

To a stirred solution of 31e (820 mg, 2.484 mmol, 1 equiv) and CI-17 (1.07 g, 3.229 mmol, 1.3 equiv) in N,N-dimethylacetamide (10 mL) were added 1H-imidazole-4-carbonitrile (46.24 mg, 0.497 mmol, 0.2 equiv) and trifluoroacetic acid (56.64 mg, 0.497 mmol, 0.2 equiv) in portions at room temperature under nitrogen atmosphere. The resulting mixture was stirred for 3 h at room temperature under nitrogen atmosphere. Desired product could be detected by LCMS. The resulting mixture was extracted with ethyl acetate (3×40 mL). The combined organic layers were washed with brine (3×30 mL), dried over anhydrous sodium sulfate. After filtration, the filtrate was concentrated under reduced pressure. The resulting mixture was concentrated under reduced pressure. The residue was purified by reversed-phase flash chromatography with the following conditions: column, C18 silica gel; mobile phase, acetonitrile in water (0.1% formic acid), 10% to 78% gradient in 30 min; detector, UV 254 nm. The resulting mixture was concentrated under reduced pressure to afford 31f (250 mg, 22.19%) as a yellow solid. LCMS (ESI, m/z): [M+H]⁺ 454.1.

### Step F. methyl 4-[3-({3-[4-({7-[1-(1-ethoxyethyl)pyrazol-4-yl]-8-isopropoxy-[1,2,4]triazolo[1,5-c]pyrimidin-2-yl}amino)-3-fluorobenzenesulfonyl]phenyl}methyl)cyclobutyl]benzoate (31g)

To a stirred solution of methyl 31f (240 mg, 0.529 mmol, 1 equiv) and CI-1 (251.00 mg, 0.635 mmol, 1.2 equiv) in dioxane (3 mL) were added Pd₂(dba)₃ (48.46 mg, 0.053 mmol, 0.1 equiv) and cesium carbonate (517.25 mg, 1.587 mmol, 3 equiv) in portions at room temperature under nitrogen atmosphere. The resulting mixture was stirred for 1 h at 90° C. under nitrogen atmosphere. Desired product could be detected by LCMS. The resulting mixture was extracted with ethyl acetate (3×20 mL). The combined organic layers were washed with brine (3×10 mL), dried over anhydrous sodium sulfate. After filtration, the filtrate was concentrated under reduced pressure. The resulting mixture was concentrated under reduced pressure. The residue was purified by silica gel column chromatography, eluted with petroleum ether/ethyl acetate(3:1) to afford 31g (320 mg, 78.75%) as a yellow solid. LCMS (ESI, m/z): [M+H]⁺ 768.2.

### Step G. 4-[3-({3-[4-({7-[1-(1-ethoxyethyl)pyrazol-4-yl]-8-isopropoxy-[1,2,4]triazolo[1,5-c]pyrimidin-2-yl}amino)-3-fluorobenzenesulfonyl]phenyl}methyl)cyclobutyl]benzoic acid (CI-31)

To a stirred solution of 31g (310 mg, 0.404 mmol, 1 equiv) was added LiOH.water (50.82 mg, 1.212 mmol, 3 equiv)in portions at room temperature under nitrogen atmosphere. The resulting mixture was stirred for 16 h at 60° C. under nitrogen atmosphere. Trace desired product was detected by LCMS, no work up was performed. The resulting mixture was concentrated under reduced pressure to afford CI-31 (300 mg, crude) as a yellow solid. The crude product was used in the next step directly without further purification. LCMS (ESI, m/z): [M+H]⁺ 754.2.

### Intermediate 32. 1-[6-(3-{[4-(4-amino-3-fluorobenzenesulfonyl)phenyl]methyl}cyclobutyl)-1-methylindazol-3-yl]-1,3-diazinane-2,4-dione (CI-32)

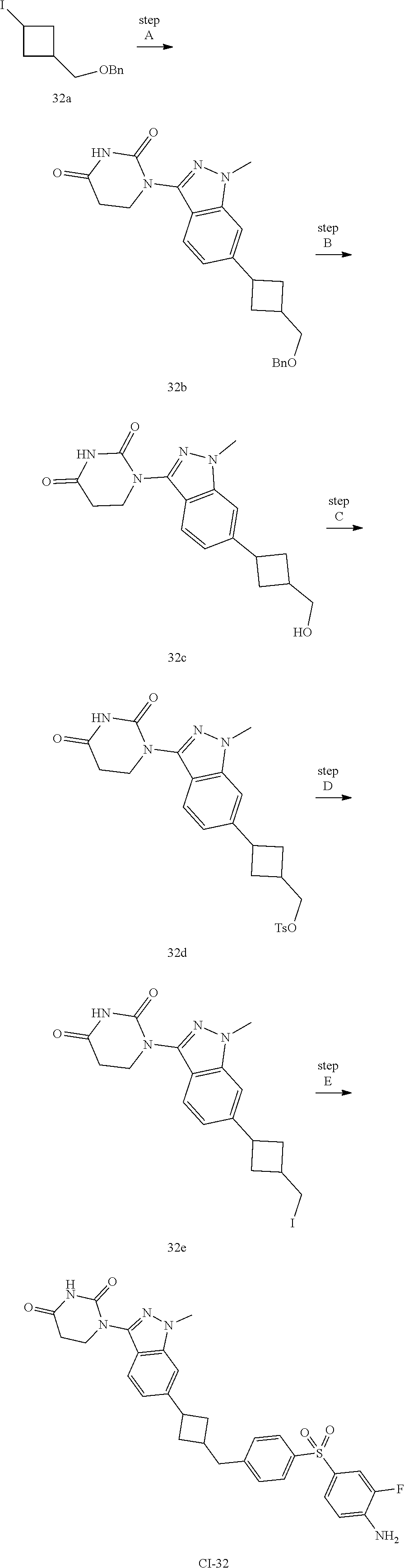

### Step A. 1-(6-{3-[(benzyloxy)methyl]cyclobutyl}-1-methylindazol-3-yl)-1,3-diazinane-2,4-dione (32b)

To a stirred solution of 10c and {[(3-iodocyclobutyl)methoxy]methyl}benzene (1.87 g, 6.190 mmol, 2 equiv) in N,N-dimethylacetamide (15 mL) were added 32a (57.61 mg, 0.619 mmol, 0.2 equiv) and trifluoroacetic acid (70.57 mg, 0.619 mmol, 0.2 equiv) in portions at room temperature under nitrogen atmosphere. The resulting mixture was stirred for 16 h at 60° C. under nitrogen atmosphere. Desired product could be detected by LCMS. The resulting mixture was extracted with ethyl acetate (3×20 mL). The combined organic layers were washed with brine (3×20 mL), dried over anhydrous sodium sulfate. After filtration, the filtrate was concentrated under reduced pressure. The residue was purified by reversed-phase flash chromatography with the following conditions: column, C18 silica gel; mobile phase, acetonitrile in water (0.1% formic acid), 10% to 59% gradient in 10 min; detector, UV 254 nm. The resulting mixture was concentrated under reduced pressure to afford 32b (400 mg, 30.89%) as a white solid. LCMS (ESI, m/z): [M+H]⁺ 419.2.

### Step B. 1-{6-[3-(hydroxymethyl)cyclobutyl]-1-methylindazol-3-yl}-1,3-diazinane-2,4-dione (32c)

To a solution of 32b (420 mg, 1.004 mmol, 1 equiv) in 10 mL ethanol was added Pd/C (10%, 400 mg) under nitrogen atmosphere in a 100 mL round-bottom flask. The mixture was hydrogenated at room temperature for 40 h under hydrogen atmosphere using a hydrogen balloon, filtered through a Celite pad and concentrated under reduced pressure. Desired product could be detected by LCMS. The resulting mixture was filtered through a celite pad and concentrated under reduced pressure to afford 32c (350 mg, crude) as a white solid. LCMS (ESI, m/z): [M+H]329.1.

### Step C. {3-[3-(2,4-dioxo-1,3-diazinan-1-yl)-1-methylindazol-6-yl]cyclobutyl}methyl 4-methylbenzenesulfonate (32d)

To a stirred solution of 32c (350 mg, 1.066 mmol, 1 equiv) and p-toluenesulfonyl chloride (406.39 mg, 2.132 mmol, 2 equiv) in dichloromethane (5 mL) were added trimethylamine (323.57 mg, 3.198 mmol, 3 equiv) and triethylenediamine (11.94 mg, 0.107 mmol, 0.1 equiv) in portions at room temperature under nitrogen atmosphere. The resulting mixture was stirred for 2 h at room temperature under nitrogen atmosphere. Desired product could be detected by LCMS. The resulting mixture was extracted with ethyl acetate (3×10 mL). The combined organic layers were washed with brine (3×10 mL), dried over anhydrous sodium sulfate. After filtration, the filtrate was concentrated under reduced pressure. The residue was purified by silica gel column chromatography, eluted with petroleum ether/ethyl acetate(1:3) to afford 32d (250 mg, 48.61%) as a white solid. LCMS (ESI, m/z): [M+H]483.1.

### Step D. 1-{6-[3-(iodomethyl)cyclobutyl]-1-methylindazol-3-yl}-1,3-diazinane-2,4-dione (32e)

To a stirred solution of 32d (250 mg, 0.518 mmol, 1 equiv) in acetone (3 mL) was added sodium iodide (388.28 mg, 2.590 mmol, 5.00 equiv) in portions at room temperature under nitrogen atmosphere. The resulting mixture was stirred for 1 h at 100° C. under nitrogen atmosphere. Desired product could be detected by LCMS. The resulting mixture was extracted with ethyl acetate (3×10 mL). The combined organic layers were washed with brine (3×10 mL), dried over anhydrous sodium sulfate. After filtration, the filtrate was concentrated under reduced pressure. The residue was purified by silica gel column chromatography, eluted with petroleum ether/ethyl acetate(1:3) to afford 32e (220 mg, 96.89%) as a yellow solid. LCMS (ESI, m/z): [M+H]⁺ 439.0.

### Step E. 1-[6-(3-{[4-(4-amino-3-fluorobenzenesulfonyl)phenyl]methyl}cyclobutyl)-1-methylindazol-3-yl]-1,3-diazinane-2,4-dione (CI-32)

To a stirred solution of 32e (220 mg, 0.502 mmol, 1 equiv) and CI-17 (331.47 mg, 1.004 mmol, 2 equiv) in N,N-dimethylacetamide (3 mL) were added 1H-imidazole-4-carbonitrile (9.35 mg, 0.100 mmol, 0.2 equiv) and trifluoroacetic acid (11.45 mg, 0.100 mmol, 0.2 equiv)in portions at room temperature under nitrogen atmosphere. The resulting mixture was stirred for 3 h at 60° C. under nitrogen atmosphere. Desired product could be detected by LCMS. The resulting mixture was extracted with ethyl acetate (3×15 mL). The combined organic layers were washed with brine (3×15 mL), dried over anhydrous sodium sulfate. After filtration, the filtrate was concentrated under reduced pressure. The residue was purified by silica gel column chromatography, eluted with petroleum ether/ethyl acetate(2:1) to afford CI-32 (70 mg, 24.83%) as a white solid. LCMS (ESI, m/z): [M+H]⁺ 562.1.

### Intermediate 33. benzyl (4-((3-bromophenyl)sulfonyl)-2-fluorophenyl)carbamate (CI-33)

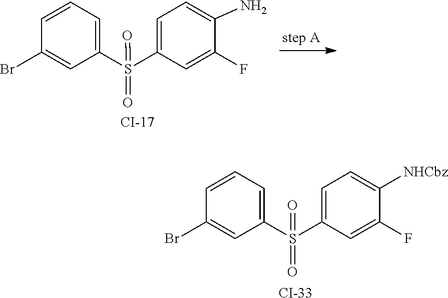

### Step A: benzyl (4-((3-bromophenyl)sulfonyl)-2-fluorophenyl)carbamate (CI-33)

To a stirred mixture of CI-17 (100 mg, 0.218 mmol, 1 equiv) and triethylamine (90.41 mg, 0.654 mmol, 4 equiv) in N,N-dimethylformamide (2.5 mL) was added benzyl carbonochloridate (30.95 mg, 0.218 mmol, 2 equiv) in portions at 0° C. The resulting mixture 20 was stirred for 16 h at room temperature. Desired product could be detected by LCMS. The reaction was quenched by the addition of water (10 mL). The resulting mixture was extracted with dichloromethane. The combined organic layers were dried over anhydrous sodium sulfate. After filtration, the filtrate was concentrated under reduced pressure. The residue was purified by silica gel column chromatography, eluted with petroleum ether/acetic ester (10:1) to afford CI-33 (100 mg, 70.99%) as a white solid. LCMS (ES, m/z): [M+H]⁺ 464.0.

### Intermediate 34. 3-(4-{[(Benzyloxy)carbonyl]aminol-3-fluorobenzenesulfonyl)phenylboronic acid (CI-34)

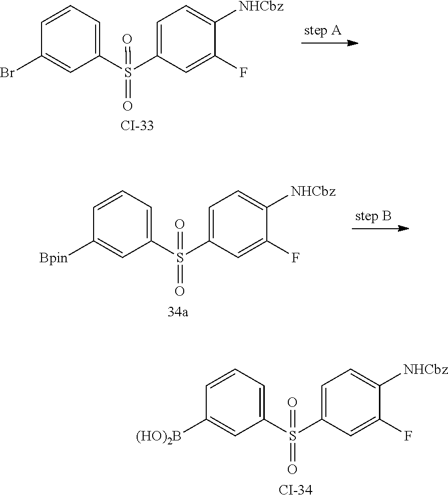

### Step A. Benzyl N-{2-fluoro-4-[3-(4,4,5,5-tetramethyl-1,3,2-dioxaborolan-2-yl)benzenesulfonyl]phenyl}carbamate (34a)

A solution of CI-33 (500 mg, 1.077 mmol, 1 equiv) and bis(pinacolato)diboron (820.38 mg, 3.231 mmol, 3 equiv), Pd(dppf)C₁₋₂ (78.80 mg, 0.108 mmol, 0.1 equiv) and potassium acetate (369.90 mg, 3.769 mmol, 3.5 equiv) in dioxane (5 mL) was stirred for 1 h at 90° C. under argon atmosphere. Desired product could be detected by LCMS. The resulting mixture was diluted with water. The resulting mixture was extracted with ethyl acetate. The combined organic layers were dried over anhydrous sodium sulfate. After filtration, the filtrate was concentrated under reduced pressure. The residue was purified by silica gel column chromatography, eluted with petroleum ether/ethyl acetate(2:1) to afford 34a (450 mg, 81.72%) as a colorless semi-solid. LCMS (ESI, m/z): [M+H]512.4

### Step B. 3-(4-{[(Benzyloxy)carbonyl]amino}-3-fluorobenzenesulfonyl)phenylboronic acid (CI-34)

To a stirred solution of 34a (450 mg, 0.880 mmol, 1 equiv) in tetrahydrofuran (8 mL)/water (2 mL) was added sodium periodate (677.60 mg, 3.168 mmol, 3.6 equiv) in portions at room temperature. The resulting mixture was stirred for 1 h at room temperature. To the above mixture was added 1 N HCl (879.99 uL, 0.880 mmol, 1 equiv) at room temperature. The resulting mixture was stirred for additional 1 h at room temperature. The resulting mixture was extracted with dichloromethane. The combined organic layers were dried over anhydrous sodium sulfate. After filtration, the filtrate was concentrated under reduced pressure. The residue was purified by Prep-TLC (dichloromethane/methanol 10:1) to afford CI-34 (220 mg, 58.24%) as a colorless semi-solid. LCMS (ESI, m/z): [M+H]⁺ 430.2

### Intermediate 35. 3-(5-{2-[3-(4-Amino-3-fluorobenzenesulfonyl)phenyl]-2,6-diazabicyclo[3.2.0]heptan-6-yl}-1-oxo-3H-isoindol-2-yl)-1-{[2-(trimethylsilyl)ethoxylmethyl]piperidine-2,6-dione (CI-35)

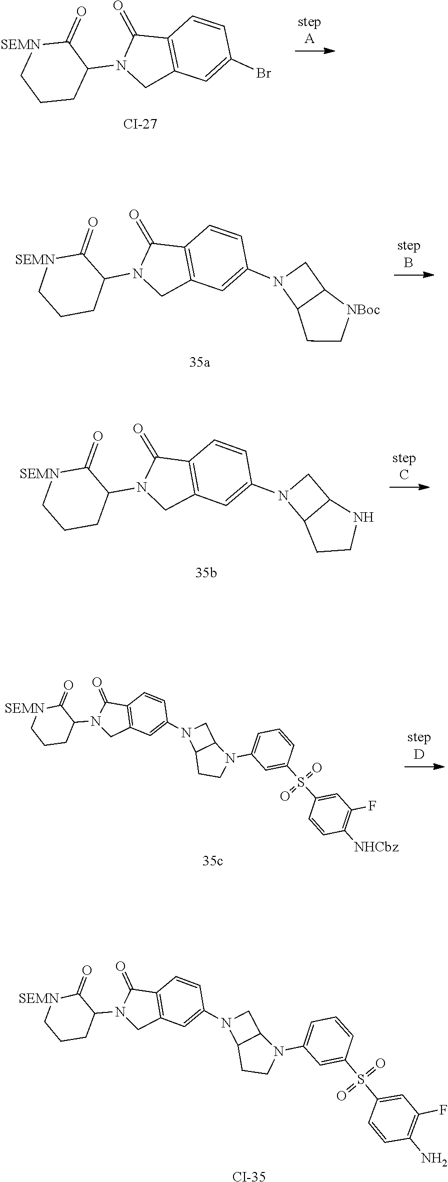

### Step A. 4-{3-[(benzyloxy)methyl]cyclobutyl}benzoate (35a)

A solution of CI-27(300 mg, 0.662 mmol, 1 equiv) and tert-butyl 2,6-diazabicyclo[3.2.0]heptane-2-carboxylate hydrochloride (186.36 mg, 0.794 mmol, 1.2 equiv), Ruphos Pd G4 (56.27 mg, 0.066 mmol, 0.1 equiv), RuPhos (30.88 mg, 0.066 mmol, 0.10 equiv) and cesium carbonate (646.74 mg, 1.986 mmol, 3.00 equiv) in dioxane (5 mL) was stirred for 1 h at 80° C. under argon atmosphere. Desired product could be detected by LCMS. The resulting mixture was concentrated under reduced pressure. The residue was purified by Prep-TLC (petroleum ether/ethyl acetatel:2) to afford 35a (300 mg, 79.44%) as alight yellow solid. LCMS (ESI, m/z): [M+H]⁺ 571.8

### Step B. 3-(5-{2,6-Diazabicyclo[3.2.0]heptan-6-yl}-1-oxo-3H-isoindol-2-yl)-1-{[2-(trimethylsilyl)ethoxy]methyl}piperidine-2,6-dione (35b)

A solution of 35a (95 mg, 0.166 mmol, 1 equiv) and p-toluenesulfonic acid (372.60 mg, 2.158 mmol, 13 equiv) in EA (2 mL) was stirred for 2 h at room temperature. The residue was purified by Prep-TLC (dichloromethane/methanol 10:1) to afford 35b (60 mg, 76.59%) as a yellow solid. LCMS (ESI, m/z): [M+H]⁺ 471.6

### Step C. Benzyl N-[4-(3-{6-[2-(2,6-dioxo-1-{[2-(trimethylsilyl)ethoxy]methyl}piperidin-3-yl)-1-oxo-3H-isoindol-5-yl]-2,6-diazabicyclo[3.2.0]heptan-2-yl}benzenesulfonyl)-2-fluorophenyl]carbamate (35c)

A solution of 35b (150 mg, 0.319 mmol, 1 equiv) and CI-34 (273.60 mg, 0.638 mmol, 2 equiv), copper acetate (115.78 mg, 0.638 mmol, 2 equiv) and triethylamine (161.26 mg, 1.595 mmol, 5 equiv), 4A MS in dichloromethane (5 mL) was stirred for 1 h at room temperature under argon atmosphere. Desired product could be detected by LCMS. The resulting mixture was extracted with dichloromethane. The combined organic layers were dried over anhydrous sodium sulfate. After filtration, the filtrate was concentrated under reduced pressure. The residue was purified by Prep-TLC (dichloromethane/methanol 20:1) to afford benzyl 35c (90 mg, 33.06%) as a yellow solid. LCMS (ESI, m/z): [M+H]⁺ 854.0

### Step D. 3-(5-{2-[3-(4-Amino-3-fluorobenzenesulfonyl)phenyl]-2,6-diazabicyclo[3.2.0]heptan-6-yl}-1-oxo-3H-isoindol-2-yl)-1-{[2-(trimethylsilyl)ethoxy]methyl}piperidine-2,6-dione (CI-35)

To a solution of 35c (120 mg, 0.141 mmol, 1 equiv) in trifluoroethanol (3 mL) was added Pd(OH)₂/C (30 mg, 0.214 mmol, 1.52 equiv) under nitrogen atmosphere. The mixture was hydrogenated at room temperature for 2 h under hydrogen atmosphere using a hydrogen balloon, filtered through a celite pad and concentrated under reduced pressure. The residue was purified by Prep-TLC (dichloromethane/methanol 20:1) to afford CI-35 (60 mg, 59.32%) as an off-white semi-solid. LCMS (ESI, m/z): [M+H]⁺ 720.9.

### Intermediate 36. Benzyl N-[4-(3-{2,7-diazabicyclo[4.2.0]octan-2-yl}benzenesulfonyl)-2-fluorophenyl]carbamate (CI-36)

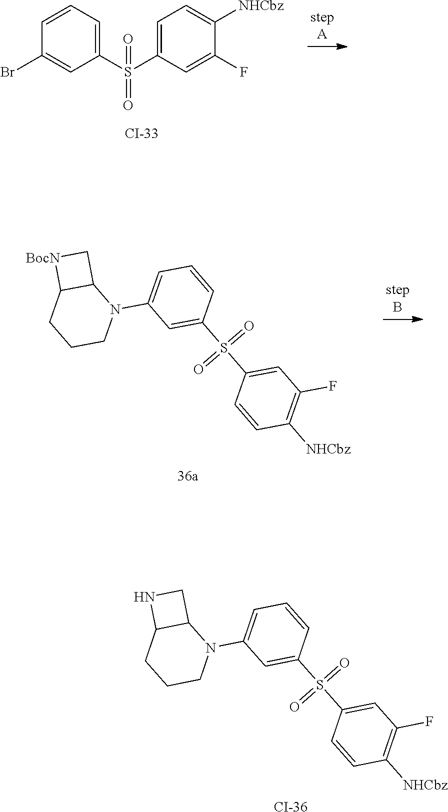

### Step A. Tert-butyl 2-[3-(4-{[(benzyloxy)carbonyl]amino}-3-fluorobenzenesulfonyl)phenyl]-2,7-diazabicyclo[4.2.0]octane-7-carboxylate (36a)

A solution of CI-33 (80 mg, 0.172 mmol, 1 equiv) and tert-butyl 2,7-diazabicyclo[4.2.0]octane-7-carboxylate (43.89 mg, 0.206 mmol, 1.2 equiv), Ruphos Pd G4 (14.65 mg, 0.017 mmol, 0.1 equiv), Ruphos (8.04 mg, 0.017 mmol, 0.1 equiv) and cesium carbonate (112.28 mg, 0.344 mmol, 2 equiv) in dioxane (2 mL) was stirred for 1 h at 80° C. under argon atmosphere. The resulting mixture was diluted with water. The resulting mixture was extracted with ethyl acetate. The combined organic layers were dried over anhydrous sodium sulfate. After filtration, the filtrate was concentrated under reduced pressure. The residue was purified by Prep-TLC (petroleum ether/ethyl acetate1:1) to afford 36a (60 mg, 58.46%) as a yellow solid. LCMS (ESI, m/z): [M+H]⁺ 596.2

### Step B. Benzyl N-[4-(3-{2,7-diazabicyclo[4.2.0]octan-2-yl}benzenesulfonyl)-2-fluorophenyl]carbamate (CI-36)

To a stirred solution of 36a (60 mg, 0.101 mmol, 1 equiv) in acetonitrile (2 mL) was added 4M HCl(gas)in 1,4-dioxane (0.4 mL, 1.600 mmol, 15.89 equiv) dropwise at 0° C. The resulting mixture was stirred for 1 h at 0° C. Desired product could be detected by LCMS. The resulting mixture was concentrated under reduced pressure. The resulting mixture was added dichloromethane. The mixture was neutralized with triethylamine. The residue was purified by Prep-TLC (dichloromethane/methanol 20:1) to afford CI-36 (40 mg, 80.14%) as an off-white solid. LCMS (ESI, m/z): [M+H]⁺ 496.2

### Intermediate 37. 3-(5-{2-[3-(4-Amino-3-fluorobenzenesulfonyl)phenyl]-2,6-diazabicyclo[3.2.0]heptan-6-yl}-1-oxo-3H-isoindol-2-yl)-1-{[2-(trimethylsilyl)ethoxylmethyl]piperidine-2,6-dione (CI-37)

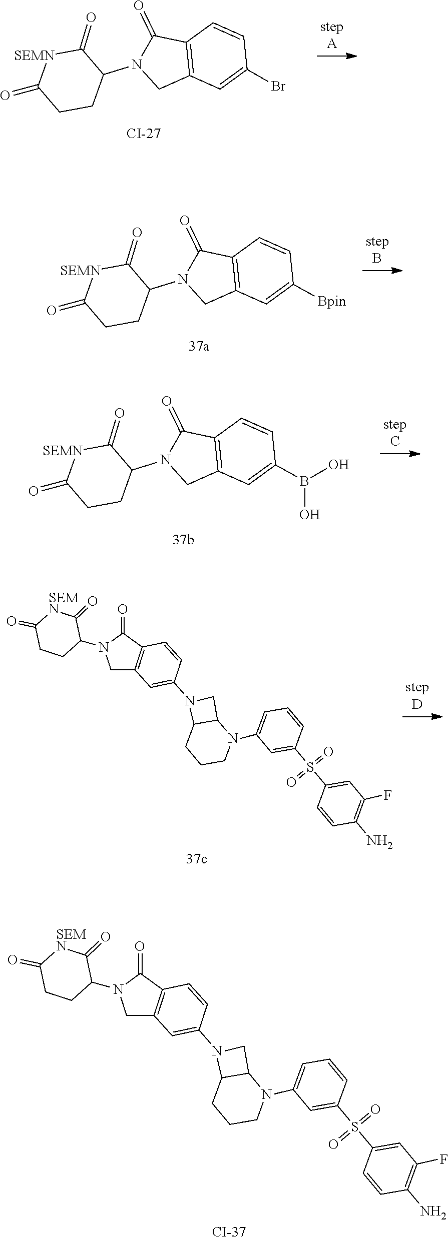

### Step A. 3-[1-oxo-5-(4,4,5,5-tetramethyl-1,3,2-dioxaborolan-2-yl)-3H-isoindol-2-yl]-1-{[2-(trimethylsilyl)ethoxy]methyl}piperidine-2,6-dione (37a)

A solution of CI-27 (400 mg, 0.882 mmol, 1 equiv) and bis(pinacolato)diboron (672.08 mg, 2.646 mmol, 3 equiv), Pd(dppf)Cl₂ (64.55 mg, 0.088 mmol, 0.1 equiv) and potassium acetate (303.04 mg, 3.087 mmol, 3.5 equiv) in dioxane (10 mL) was stirred for 1 h at 90° C. under argon atmosphere. Desired product could be detected by LCMS. The resulting mixture was concentrated under reduced pressure. The residue was purified by silica gel column chromatography, eluted with petroleum ether/ethyl acetate(1:1) to afford 37a (370 mg, 83.80%) as an off-white semi-solid. LCMS (ESI, m/z): [M+H]⁺ 501.5

### Step B. 2-(2,6-Dioxo-1-{[2-(trimethylsilyl)ethoxy]methyl}piperidin-3-yl)-1-oxo-3H-isoindol-5-ylboronic acid (37b)

To a stirred solution of 37a (5 g, 9.991 mmol, 1 equiv) in tetrahydrofuran (40 mL)/water (10 mL) was added sodium periodate (7.69 g, 35.968 mmol, 3.6 equiv) in portions at room temperature. The resulting mixture was stirred for 1 h at room temperature. To the above mixture was added 1N HCl (9.99 mL, 9.991 mmol, 1 equiv) at room temperature. The resulting mixture was stirred for additional 1 h at room temperature. The resulting mixture was extracted with dichloromethane. The combined organic layers were dried over anhydrous sodium sulfate. After filtration, the filtrate was concentrated under reduced pressure. The residue was purified by reversed-phase flash chromatography with the following conditions: column, C18 silica gel; mobile phase, acetonitrile in water (0.1% formic acid), 10% to 100% gradient in 30 min; detector, UV 254 nm. This resulted in 37b (2.5 g, 59.82%) as a brown semi-solid. LCMS (ESI, m/z): [M+H]⁺ 419.3

### Step C. Benzyl N-[4-(3-{7-[2-(2,6-dioxo-1-{[2-(trimethylsilyl)ethoxy]methyl}piperidin-3-yl)-1-oxo-3H-isoindol-5-yl]-2,7-diazabicyclo[4.2.0]octan-2-yl}benzenesulfonyl)-2-fluorophenyl]carbamate (37c)

A solution of 37b (130.00 mg, 0.310 mmol, 2.5 equiv) and CI-36 (61.6 mg, 0.124 mmol, 1.00 equiv), copper acetate (45.15 mg, 0.248 mmol, 2 equiv) and triethylamine (62.89 mg, 0.620 mmol, 5 equiv) in dichloromethane (5 mL) was stirred for 3 h at room temperature under air atmosphere. Desired product could be detected by LCMS. The residue was purified by Prep-TLC (dichloromethane/methanol 20:1) to afford 37c (80 mg, 74.14%) as an off-white solid. LCMS (ESI, m/z): [M+H]868.0

### Step D. 3-(5-{2-[3-(4-Amino-3-fluorobenzenesulfonyl)phenyl]-2,7-diazabicyclo[4.2.0]octan-7-yl}-1-oxo-3H-isoindol-2-yl)-1-{[2-(trimethylsilyl)ethoxy]methyl}piperidine-2,6-dione (CI-37)

To a solution of 37c (80 mg, 0.092 mmol, 1 equiv) in 2,2,2-trifluoroethanol (3 mL) was added Pd(OH)₂/C (20 mg) under nitrogen atmosphere. The mixture was hydrogenated at room temperature for 1 h under hydrogen atmosphere using a hydrogen balloon, filtered through a Celite pad and concentrated under reduced pressure. The residue was purified by Prep-TLC (dichloromethane/methanol 20:1) to afford CI-37 (45 mg, 66.53%) as an off-white solid. LCMS (ESI, m/z): [M+H]734.0

### Intermediate 38. 3-(3-methyl-2-oxo-5-(4-oxopiperidin-1-yl)-2,3-dihydro-1H-benzo[d]imidazol-1-yl)-1-((2-(trimethylsilyl) ethoxy)methyl)piperidine-2,6-dione (CI-38)

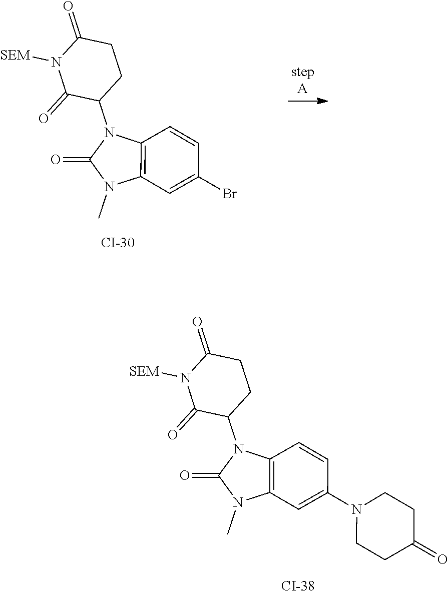

### Step A: 3-(3-methyl-2-oxo-5-(4-oxopiperidin-1-yl)-2,3-dihydro-1H-benzo[d]imidazol-1-yl)-1-((2-(trimethylsilyl)ethoxy)methyl)piperidine-2,6-dione (CI-38)

To a stirred mixture of CI-30 (100 mg, 0. 31 mmol, 1 equiv) and 4-piperidinone (36.8 mg, 0.37 mmol, 1.2 equiv) in N,N-dimethylformamide (5 mL) was added cesium carbonate (403.3 mg, 1.24 mmol, 4 equiv) and RuPhos Palladacycle Gen.3 (38.8 mg, 0.005 mmol, 0.15 equiv). The resulting mixture was stirred for 12 h at 80° C. under air atmosphere. Desired product could be detected by LCMS. The reaction was quenched by the addition of water (5 mL). The resulting mixture was extracted with ethyl acetate. The combined organic layers were dried over anhydrous sodium sulfate. After filtration, the filtrate was concentrated under reduced pressure. The residue was purified by silica gel column chromatography, eluted with petroleum ether/ethyl acetate (2:1) to afford CI-38 (80 mg, 76.87%) as a white solid. LCMS (ESI, m/z): [M+H]⁺ 487.2.

### Intermediate 39. N-(4-((2,7-diazaspiro[4.41nonan-2-yl)sulfonyl)-2-fluorophenyl)-8-isopropoxy-7-(1H-pyrazol-4-yl)-[1,2,4]triazolo[1,5-c]pyrimidin-2-amine (CI-39)

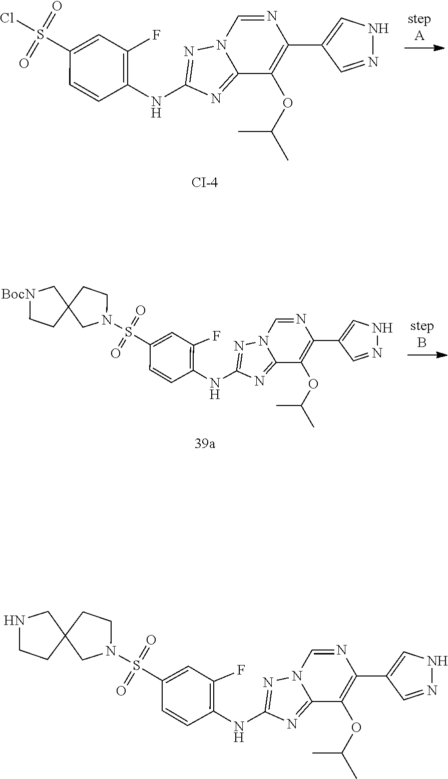 [SMILES: CC(C)Oc1c(-c2cn[nH]c2)ncn2nc(Nc3ccc(S(=O)(=O)Cl)cc3F)nc12 ; "CI-4" ; CC(C)Oc1c(-c2cn[nH]c2)ncn2nc(Nc3ccc(S(=O)(=O)N4CCC5(CCN(C(=O)OC(C)(C)C)C5)C4)cc3F)nc12 ; CC(C)Oc1c(-c2cn[nH]c2)ncn2nc(Nc3ccc(S(=O)(=O)N4CCC5(CCNC5)C4)cc3F)nc12]

### Step A. tert-butyl 7-(3-fluoro-4-{1[8-isopropoxy-7-(1H-pyrazol-4-yl)-[1,2,4]triazolo[1,5-c]pyrimidin-2-yl]amino}benzenesulfonyl)-2,7-diazaspiro[4.4]nonane-2-carboxylate) (39a)

To a stirred mixture of CI-4 (150 mg, 0.332 mmol, 1 equiv) and trimethylamine (167.96 mg, 1.660 mmol, 5 equiv) in acetonitrile (2 mL) was added tert-butyl 2,7-diazaspiro[4.4]nonane-2-carboxylate (90.16 mg, 0.398 mmol, 1.2 equiv) in portions at 0° C. The resulting mixture was stirred for 30 min at 0° C. The reaction was monitored by TLC. The resulting mixture was concentrated under reduced pressure. The residue was purified by silica gel column chromatography, eluted with petroleum ether/ethyl acetate(1:1) to afford 39a as a white solid. LCMS (ESI, m/z): [M+H]⁺ 642.2.

### Step B. N-(4-((2,7-diazaspiro[4.4]nonan-2-yl)sulfonyl)-2-fluorophenyl)-8-isopropoxy-7-(1H-pyrazol-4-yl)-[1,2,4]triazolo[1,5-c]pyrimidin-2-amine (CI-39)

To a stirred mixture of 39a (100 mg, 0.156 mmol, 1 equiv) in dichloromethane (2 mL) was added trifluoroacetic acid (2 mL) in portions at 0° C. The resulting mixture was stirred for 1 h at room temperature. The reaction was monitored by LCMS. The resulting mixture was concentrated under reduced pressure. The crude product was used in the next step directly without further purification. LCMS (ESI, m/z): [M+H]⁺ 542.2.

### Intermediate 40. 3-(5-(4-(1,7-diazaspiro[4.4]nonan-1-yl)piperidin-1-yl)-3-methyl-2-oxo-2,3-dihydro-1H-benzo[d]imidazol-1-yl)piperidine-2,6-dione (CI-40)

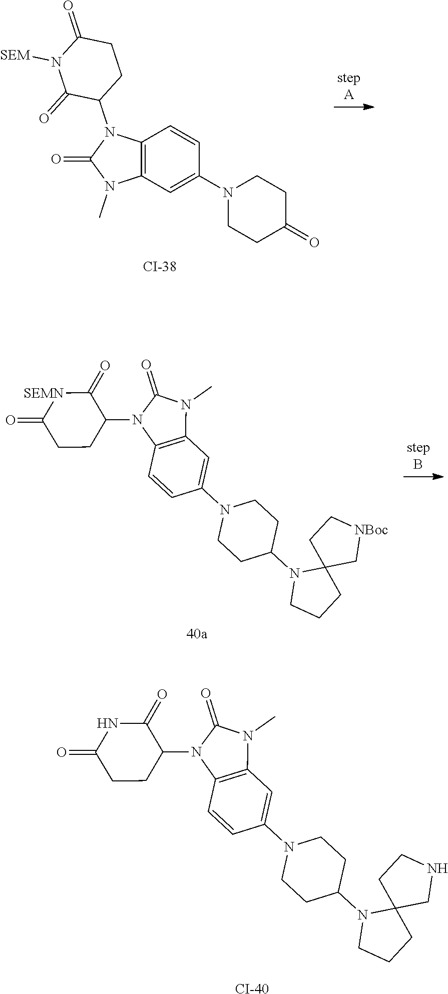 [SMILES: Cn1c(=O)n(C2CCC(=O)N(COCC[Si](C)(C)C)C2=O)c2ccc(N3CCC(=O)CC3)cc21 ; "CI-38" ; Cn1c(=O)n(C2CCC(=O)N(COCC[Si](C)(C)C)C2=O)c2ccc(N3CCC(N4CCCC45CCN(C(=O)OC(C)(C)C)C5)CC3)cc21 ; Cn1c(=O)n(C2CCC(=O)NC2=O)c2ccc(N3CCC(N4CCCC45CCNC5)CC3)cc21 ; "CI-40"]

### Step A. tert-butyl 1-(1-(1-(2,6-dioxo-1-((2-(trimethylsilyl)ethoxy)methyl)piperidin-3-yl)-3-methyl-2-oxo-2,3-dihydro-1H-benzo[d]imidazol-5-yl)piperidin-4-yl)-1,7-diazaspiro[4.4]nonane-7-carboxylate (40a)

To a stirred mixture of CI-38 (110 mg, 0.226 mmol, 1 equiv) and tert-butyl 1,7-diazaspiro[4.4]nonane-1-carboxylate (153.47 mg, 0.678 mmol, 3 equiv) in N,N-dimethylformamide (1.5 mL) were added sodium cyanoborohydride (56.82 mg, 0.904 mmol, 4 equiv) and acetic acid (1.36 mg, 0.023 mmol, 0.1 equiv) in portions at 50° C. The resulting mixture was stirred for overnight at 50° C. The reaction was monitored by LCMS. The residue was purified by reversed-phase flash chromatography with the following conditions: column, C18 silica gel; mobile phase, acetonitrile in water (10 mmol/L ammonium bicarbonate), 10% to 50% gradient in 10 min; detector, UV 254 nm. This resulted in 40a (50 mg, 31.74%) as a white solid. LCMS (ESI, m/z): [M+H]⁺ 697.4.

### Step B. 3-(5-(4-(1,7-diazaspiro[4.4]nonan-1-yl)piperidin-1-yl)-3-methyl-2-oxo-2,3-dihydro-1H-benzo[d]imidazol-1-yl)piperidine-2,6-dione (CI-40)

A mixture of 40a (70 mg, 0.100 mmol, 1 equiv) and trifluoroacetic acid (2 mL) in dichloromethane (2 mL) was stirred for 1 h at 0° C. The reaction was monitored by LCMS. The solvent was removed under reduced pressure. This resulted in CI-40 (60 mg) as a crude product. The crude product was used in the next step directly without further purification. LCMS (ESI, m/z): [M+H]⁺ 467.2.

### Intermediate 41. 1-[1-methyl-6-(piperazin-1-yl)indazol-3-yl]-1,3-diazinane-2,4-dione (CI-41)

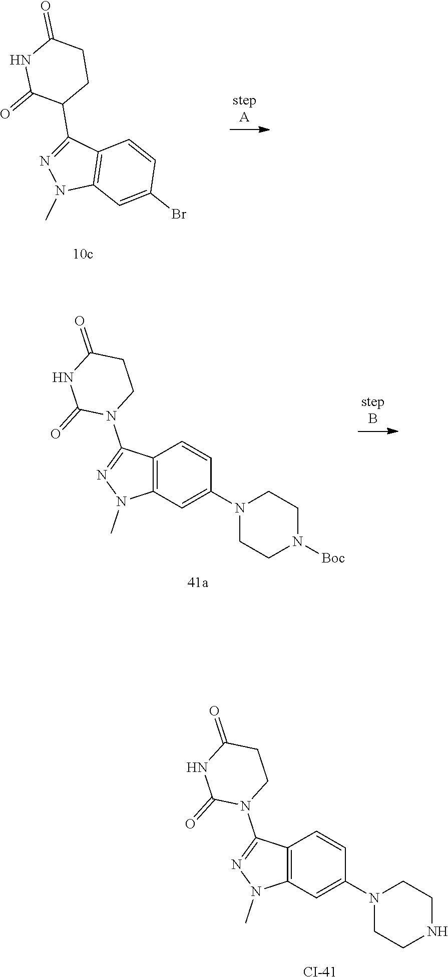

### Step A. tert-butyl 4-[3-(2,4-dioxo-1,3-diazinan-1-yl)-1-methylindazol-6-yl]piperazine-1-carboxylate (41a)

To a stirred solution of 10c (0.86 g, 4.643 mmol, 1.5 equiv) in N,N-dimethylformamide (15 mL) were added RuPhos Pd G3 (0.26 g, 0.310 mmol, 0.1 equiv) and cesium carbonate (3.02 g, 9.285 mmol, 3 equiv) in portions at room temperature under nitrogen atmosphere. The resulting mixture was stirred for 16 h at 100° C. under nitrogen atmosphere. Desired product could be detected by LCMS. The resulting mixture was extracted with ethyl acetate (3×30 mL). The combined organic layers were washed with brine (3×30 mL), dried over anhydrous sodium sulfate. After filtration, the filtrate was concentrated under reduced pressure. The residue was purified by reversed-phase flash chromatography with the following conditions: column, C18 silica gel; mobile phase, acetonitrile in water (10 mmol/L ammonium bicarbonate), 10% to 35% gradient in 30 min; detector, UV 254 nm. The resulting mixture was concentrated under reduced pressure to afford 41a (430 mg, 32.43%) as a white solid. LCMS (ESI, m/z): [M+H]⁺ 429.2.

### Step B. 1-[1-methyl-6-(piperazin-1-yl)indazol-3-yl]-1,3-diazinane-2,4-dione (CI-41)

To a stirred solution of 41a (430 mg, 1.004 mmol, 1 equiv)in acetonitrile (8 mL) was added HCl(gas)in 1,4-dioxane (4 mL) dropwise at 0° C. under hydrogen chloride atmosphere. The resulting mixture was stirred for 1 h at 0° C. under Hydrogen chloride atmosphere. Desired product could be detected by LCMS. The resulting mixture was concentrated under reduced pressure. The residue was purified by reversed-phase flash chromatography with the following conditions: column, C18 silica gel; mobile phase, acetonitrile in water (10 mmol/L ammonium bicarbonate), 10% to 20% gradient in 30 min; detector, UV 254 nm. The resulting mixture was concentrated under reduced pressure to afford CI-41 (250 mg, 75.87%) as a white solid. LCMS (ESI, m/z): [M+H]⁺ 329.1.

### Intermediate 42. rac-(R)-1-(1-methyl-6-(3-methylpiperazin-1-yl)-1H-indazol-3-yl)dihydropyrimidine-2,4(1H,3H)-dione (CI-42)

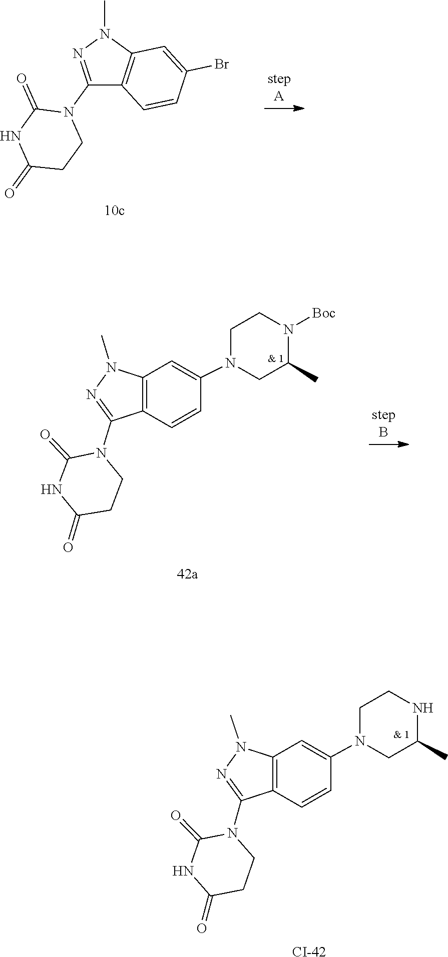 [SMILES: Cn1nc(N2CCC(=O)NC2=O)c2ccc(Br)cc21 ; C[C@H]1CN(c2ccc3c(N4CCC(=O)NC4=O)nn(C)c3c2)CCN1C(=O)OC(C)(C)C ; C[C@H]1CN(c2ccc3c(N4CCC(=O)NC4=O)nn(C)c3c2)CCN1 ; "CI-42"]

### Step A. tert-butyl 4-[3-(2,4-dioxo-1,3-diazinan-1-yl)-1-methylindazol-6-yl]piperazine-1-carboxylate (42a)

To a stirred mixture of 10c (536 mg, 1.659 mmol, 1 equiv) and tert-butyl (2S)-2-methylpiperazine-1-carboxylate (498.30 mg, 2.489 mmol, 1.5 equiv) in N,N-dimethylformamide (7 mL) was added cesium carbonate (1621.28 mg, 4.977 mmol, 3 equiv)and RuPhos Palladacycle Gen.3 (277.46 mg, 0.332 mmol, 0.2 equiv) in portions at room temperature under nitrogen atmosphere. The solution was stirred for 18 h at 100° C. Desired product could be detected by LCMS. The resulting mixture was extracted with ethyl acetate (2×20 mL). The combined organic layers were washed with water (2×20 mL), dried over anhydrous sodium sulfate. After filtration, the filtrate was concentrated under reduced pressure. The crude product was purified by Prep-HPLC to afford 42a as a yellow solid. LCMS (ESI, m/z): [M+H]⁺ 443.2.

### Step B. rac-(R)-1-(1-methyl-6-(3-methylpiperazin-1-yl)-1H-indazol-3-yl)dihydropyrimidine-2,4 (1H,3H)-dione (CI-42)

A solution of 42a (132 mg, 0.298 mmol, 1 equiv) and HCl (gas) in 1,4-dioxane (1 mL, 32.913 mmol, 110.34 equiv) in acetonitrile (1.5 mL, 28.536 mmol, 95.67 equiv) was stirred for 1.5h at 0° C. under air atmosphere. Desired product could be detected by LCMS. The resulting mixture was concentrated under reduced pressure. The crude product mixture was used in the next step directly without further purification. LCMS (ESI, m/z): [M+H]⁺ 343.0.

### Intermediate 43. (3R,4S)-1-[3-(bromomethyl)benzenesulfonyl]-N-{7-[1-(1-ethoxyethyl)pyrazol-4-yl}-8-isopropoxy-[1,2,41triazolo[1,5-cl pyrimidin-2-yl}-3-methylpiperidin-4-amine (CI-43)

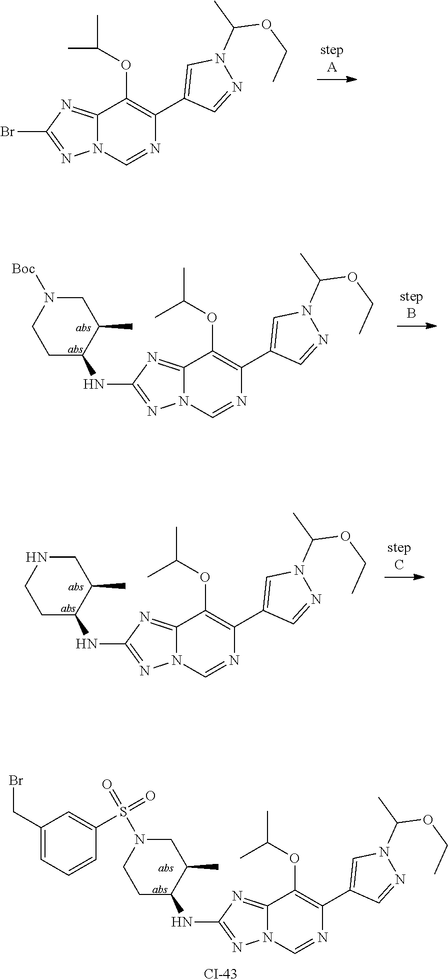 [SMILES: CCOC(C)n1cc(-c2ncn3nc(Br)nc3c2OC(C)C)cn1 ; CCOC(C)n1cc(-c2ncn3nc(N[C@H]4CCN(C(=O)OC(C)(C)C)C[C@H]4C)nc3c2OC(C)C)cn1 ; CCOC(C)n1cc(-c2ncn3nc(N[C@H]4CCNC[C@H]4C)nc3c2OC(C)C)cn1 ; CCOC(C)n1cc(-c2ncn3nc(N[C@H]4CCN(S(=O)(=O)c5cccc(CBr)c5)C[C@H]4C)nc3c2OC(C)C)cn1 ; "CI-43"]

### Step A. tert-butyl (3R,4S)-4-({7-[1-(1-ethoxyethyl)pyrazol-4-yl]-8-isopropoxy-[1,2,4]triazolo[1,5-c]pyrimidin-2-yl}amino)-3-methylpiperidine-1-carboxylate (43a)

To a stirred mixture of CI-7 (500 mg, 1.265 mmol, 1 equiv) and tert-butyl (3R,4S)-4-amino-3-methylpiperidine-1-carboxylate (325.32 mg, 1.518 mmol, 1.2 equiv) in dioxane (5 mL) were added Ruphos (118.06 mg, 0.253 mmol, 0.2 equiv), Pd₂(dba)₃ (115.84 mg, 0.127 mmol, 0.1 equiv) and cesium carbonate (1236.47 mg, 3.795 mmol, 3 equiv) in portions at room temperature under argon atmosphere. The resulting mixture was stirred for 1 h at 90° C. under argon atmosphere. Desired product could be detected by LCMS. The resulting mixture was concentrated under reduced pressure. The residue was purified by silica gel column chromatography, eluted with petroleum ether/ethyl acetate(1:1) to afford 43a (400 mg, 59.81%) as a white solid. LCMS (ESI, m/z): [M+H]⁺ 529.3.

### Step B. (3R,4S)—N-{7-[1-(1-ethoxyethyl)pyrazol-4-yl]-8-isopropoxy-[1,2,4]triazolo[1,5-c]pyrimidin-2-yl}-3-methylpiperidin-4-amine (43b)

To a stirred mixture of 43a (300 mg, 0.567 mmol, 1 equiv) and trimethylamine (287.12 mg, 2.835 mmol, 5 equiv) in dichloromethane (3 mL, 47.192 mmol, 83.16 equiv) was added trimethylsilyl trifluoromethanesulfonate (0.31 mL, 1.701 mmol, 3 equiv) in portions at 0° C. The resulting mixture was stirred for 0.5 h at 0° C. Desired product could be detected by LCMS. The reaction was quenched by the addition of sodium bicarbonate (10 mL). The resulting mixture was extracted with dichloromethane. The combined organic layers were dried over anhydrous sodium sulfate to afford 43b (350 mg, crude) as a brown oil. LCMS (ESI, m/z): [M+H]⁺ 429.3.

### Step C. (3R,4S)-1-[3-(bromomethyl)benzenesulfonyl]-N-{7-[1-(1-ethoxyethyl)pyrazol-4-yl]-8-isopropoxy-[1,2,4]triazolo[1,5-c]pyrimidin-2-yl}-3-methylpiperidin-4-amine (CI-43)

To a stirred mixture of 43b (350 mg, 0.817 mmol, 1 equiv) and triethylethylenediamine (316.68 mg, 2.451 mmol, 3 equiv) in dichloromethane (3 mL) was added 3-(bromomethyl)benzenesulfonyl chloride (264.17 mg, 0.980 mmol, 1.2 equiv). The resulting mixture was stirred for 1 h at 25° C. Desired product could be detected by LCMS. The resulting mixture was concentrated under reduced pressure. The residue was purified by silica gel column chromatography, eluted with petroleum ether/ethyl acetate (1:2) to afford CI-43 (350 mg, 55.90%) as a white solid. LCMS (ESI, m/z): [M+H]⁺ 661.2.

### Intermediate 44. 1-{1-methyl-6-[(2S)-2-methylpiperazin-1-yl}indazol-3-yl}-1,3-diazinane-2,4-dione (CI-44)

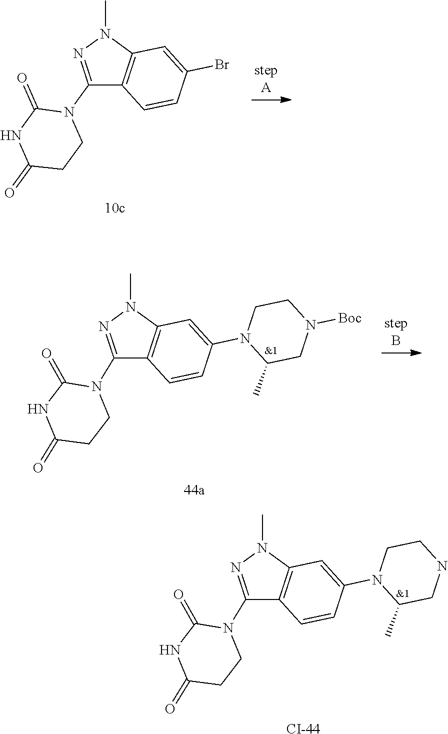 [SMILES: Cn1nc(N2CCC(=O)NC2=O)c2ccc(Br)cc21 ; C[C@H]1CN(C(=O)OC(C)(C)C)CCN1c1ccc2c(N3CCC(=O)NC3=O)nn(C)c2c1 ; C[C@H]1CNCCN1c1ccc2c(N3CCC(=O)NC3=O)nn(C)c2c1 ; "CI-44"]

### Step A. tert-butyl (3S)-4-[3-(2,4-dioxo-1,3-diazinan-1-yl)-1-methylindazol-6-yl]-3-methylpiperazine-1-carboxylate (44a)

To a solution of 10c (600 mg, 1.857 mmol, 1 equiv) in 1,4-dioxane (6 mL, 0.068 mmol, 0.04 equiv) was added tert-butyl (3S)-3-methylpiperazine-1-carboxylate (371.87 mg, 1.857 mmol, 1 equiv), Pd-PEPPSI-IHeptCl 3-chloropyridine (180.81 mg, 0.186 mmol, 0.1 equiv), sodium 2-methylpropan-2-olate (17.84 mg, 0.186 mmol, 0.1 equiv) at room temperature. The mixture was stirred for 2 min at 100° C. Desired product could be detected by LCMS. The resulting mixture was extracted with EA (3×50 mL). After filtration, the filtrate was concentrated under reduced pressure. The residue was purified by silica gel column chromatography, eluted with dichloromethane/methanol (30/1) to afford 44a (280 mg, 34.08%) as a yellow solid. LCMS (ESI, m/z): [M+H]⁺=443.2

### Step B. 1-{1-methyl-6-[(2S)-2-methylpiperazin-1-yl]indazol-3-yl}-1,3-diazinane-2,4-dione (CI-44)

To a solution of 44a (280 mg, 0.633 mmol, 1 equiv) in dichloromethane (3 mL, 47.192 mmol, 74.58 equiv) was added HCl (gas) in 1,4-dioxane (0.6 mL, 18.990 mmol, 30 equiv) at 0° C. The mixture was stirred for 30 min. Desired product could be detected by LCMS. The resulting mixture was concentrated under reduced pressure to afford CI-44 (215 mg, 90.01%) as yellow solid. LCMS (ESI, m/z): [M+H]⁺=343.2.

### Intermediate 45. rac-(R)-1-(6-(3-isopropylpiperazin-1-yl)-1-methyl-1H-indazol-3-yl)dihydropyrimidine-2,4(1H,3H)-dione (CI-45)

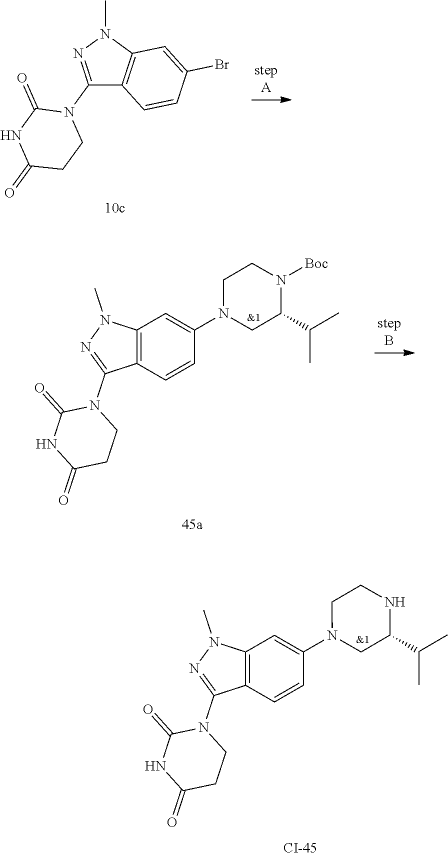 [SMILES: Cn1nc(N2CCC(=O)NC2=O)c2ccc(Br)cc21 ; CC(C)[C@@H]1CN(c2ccc3c(N4CCC(=O)NC4=O)nn(C)c3c2)CCN1C(=O)OC(C)(C)C ; CC(C)[C@@H]1CN(c2ccc3c(N4CCC(=O)NC4=O)nn(C)c3c2)CCN1 ; "CI-45"]

### Step A. rac-tert-butyl(R)-4-(3-(2,4-dioxotetrahydropyrimidin-1(2H)-yl)-1-methyl-1H-indazol-6-yl)-2-isopropylpiperazine-1-carboxylate (45a)

To a stirred mixture of 10c (400 mg, 1.238 mmol, 1 equiv) and tert-butyl (2R)-2-isopropylpiperazine-1-carboxylate (565.28 mg, 2.476 mmol, 2 equiv) in dioxane (10 mL) were added RuPhos Palladacycle Gen.3 (155.29 mg, 0.186 mmol, 0.15 equiv) and potassium carbonate (513.22 mg, 3.714 mmol, 3 equiv) and RuPhos (86.64 mg, 0.186 mmol, 0.15 equiv) at room temperature under nitrogen atmosphere. The resulting mixture was stirred for overnight at 110° C. under nitrogen atmosphere. Desired product could be detected by LCMS. The resulting mixture was diluted with water (20 mL). The resulting mixture was extracted with ethyl acetate (3×20 mL). The combined organic layers were washed with water (3×20 mL), dried over anhydrous sodium sulfate. After filtration, the filtrate was concentrated under reduced pressure. The residue was purified by silica gel column chromatography, eluted with petroleum ether/ethyl acetate(1:1) to afford 45a (230 mg, 39.49%) as a brown solid. LCMS (ESI, m/z): [M+H]⁺ 471.2.

### Step B. rac-(R)-1-(6-(3-isopropylpiperazin-1-yl)-1-methyl-1H-indazol-3-yl)dihydropyrimidine-2,4(1H,3H)-dione (CI-45)

To a stirred solution of 45a (200 mg, 0.425 mmol, 1 equiv) in acetonitrile (5 mL) was added HCl (gas) in 1,4-dioxane (1 mL, 32.913 mmol, 77.44 equiv) dropwise at 0° C. under nitrogen atmosphere. The resulting mixture was stirred for 1 h at 0° C. under nitrogen atmosphere. Desired product could be detected by LCMS. The resulting mixture was concentrated under reduced pressure. The residue was purified by reversed-phase flash chromatography with the following conditions: column, C18 silica gel; mobile phase, acetonitrile in water (10 mmol/L ammonium bicarbonate), 10% to 70% gradient in 20 min; detector, UV 254 nm. This resulted in CI-45 (130 mg, 82.57%) as a brown solid. LCMS (ESI, m/z): [M+H]⁺ 371.2.

### Intermediate 46. 1-{6-[(2R)-2-isopropylpiperazin-1-yl]-1-methylindazol-3-yl]-1,3-diazinane-2,4-dione (CI-46)

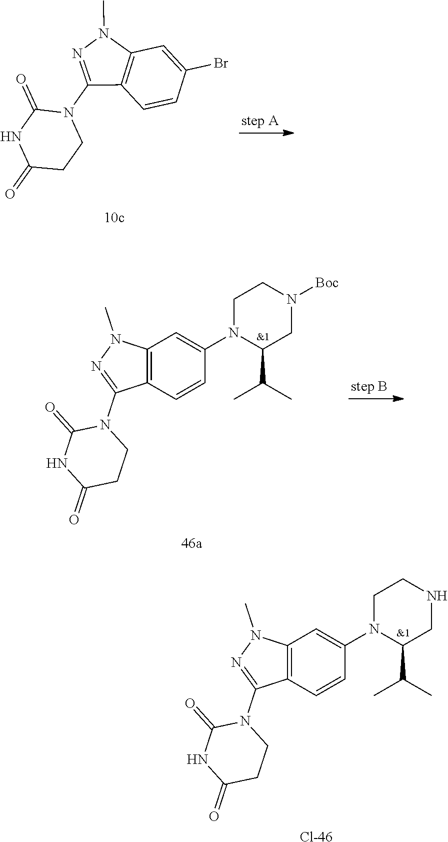

### Step A. tert-butyl (3R)-4-[3-(2,4-dioxo-1,3-diazinan-1-yl)-1-methylindazol-6-yl]-3-isopropylpiperazine-1-carboxylate (46a)

To a stirred solution of 10c (600 mg, 1.857 mmol, 1 equiv) and tert-butyl 3-isopropylpiperazine-1-carboxylate (635.94 mg, 2.785 mmol, 1.5 equiv) in dioxane (5 mL) were added Pd-PEPPSI-IPentCl 2-methylpyridine (o-picoline) (312.36 mg, 0.371 mmol, 0.2 equiv) and sodium tert-butoxide (535.32 mg, 5.571 mmol, 3 equiv) in portions at room temperature under nitrogen atmosphere. The resulting mixture was stirred for 1 h at 100° C. under nitrogen atmosphere. Desired product could be detected by LCMS. The residue was purified by reversed-phase flash chromatography with the following conditions: column, C18 silica gel; mobile phase, acetonitrile in water (10 mmol/L ammonium bicarbonate), 10% to 50% gradient in 10 min; detector, UV 254 nm. This resulted in 46a (150 mg, 17.17%) as a yellow solid. LCMS (ESI, m/z): [M+H]⁺ 471.5.

### Step B. 1-{6-[(2R)-2-isopropylpiperazin-1-yl]-1-methylindazol-3-yl}-1,3-diazinane-2,4-dione (CI-46)

A solution of 46a (10 mg, 0.021 mmol, 1 equiv) and HCl(gas)in 1,4-dioxane (1.50 mL, 49.407 mmol, 154.88 equiv) in acetonitrile (1.50 mL, 28.557 mmol, 89.52 equiv) was stirred for 1 h at 0° C. under nitrogen atmosphere. Desired product could be detected by LCMS. The residue was purified by reversed-phase flash chromatography with the following conditions: column, C18 silica gel; mobile phase, acetonitrile in water (10 mmol/L ammonium bicarbonate), 10% to 50% gradient in 10 min; detector, UV 254 nm. This resulted in CI-46 (100 mg, 84.68%) as a white solid. LCMS (ESI, m/z): [M+H]⁺ 371.4.

### Intermediate 47. 1-(6-{2,5-diazabicyclo[4.1.0]heptan-2-yl}-1-methylindazol-3-yl)-1,3-diazinane-2,4-dione (CI-47)

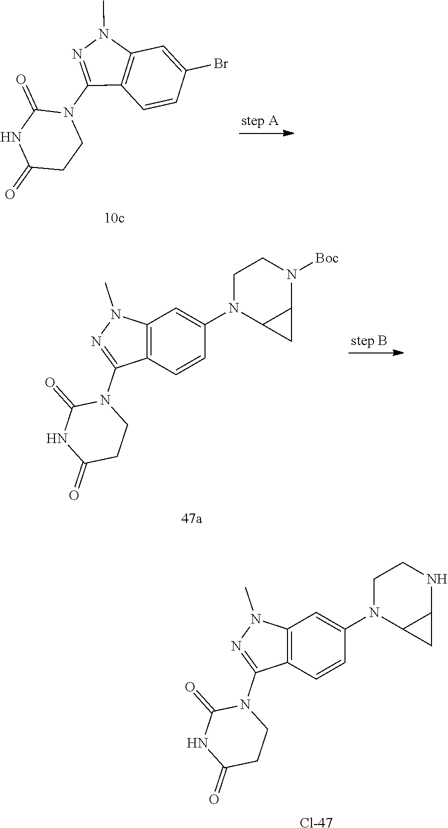

### Step A. tert-butyl 5-[3-(2,4-dioxo-1,3-diazinan-1-yl)-1-methylindazol-6-yl]-2,5-diazabicyclo[4.1.0]heptane-2-carboxylate (47a)

A solution of 10c (500 mg, 1.547 mmol, 1 equiv) and Pd PEPPSI IPentCl (133.15 mg, 0.155 mmol, 0.1 equiv) in dioxane (5 mL) was stirred for 1 h at 100° C. under argon atmosphere. Desired product could be detected by LCMS. The residue was purified by silica gel column chromatography, eluted with dichloromethane/methanol (30:1) to afford 47a (100 mg, 14.67%) as a yellow solid. LCMS (ESI, m/z): [M+H]⁺ 441.22.

### Step B. 1-(6-{2,5-diazabicyclo[4.1.0]heptan-2-yl}-1-methylindazol-3-yl)-1,3-diazinane-2,4-dione (CI-47)

A solution of 47a (100 mg, 0.227 mmol, 1 equiv) and HCl in dioxane (49.66 mg, 1.362 mmol, 4 M, 6 equiv) in dichloromethane (2 mL) was stirred for 30 min at 0° C. under nitrogen atmosphere. Desired product could be detected by LCMS. The solvent was removed under reduced pressure. This resulted in 47a (80 mg) as a crude product. The crude product was used in the next step directly without further purification. LCMS (ESI, m/z): [M+H]⁺ 341.16.

### Intermediate 48. 1-(6-(3,6-diazabicyclo[3.1.1]heptan-3-yl)-1-methyl-1H-indazol-3-yl)dihydropyrimidine-2,4(1H,3H)-dione (CI-48)

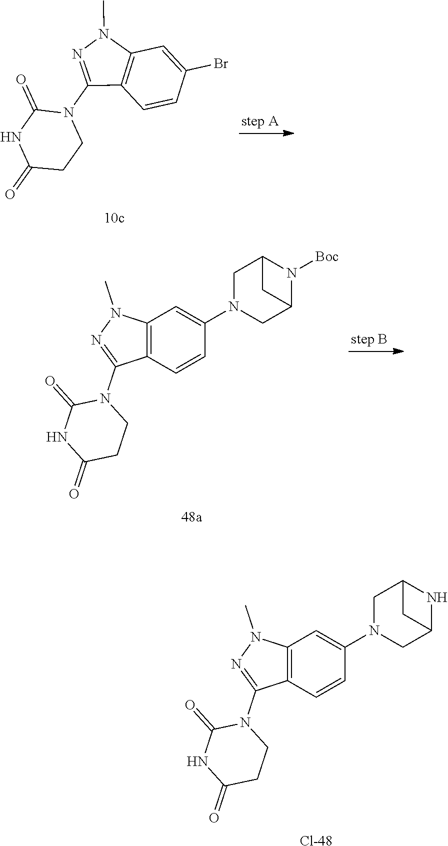

### Step A. tert-butyl 3-(3-(2,4-dioxotetrahydropyrimidin-1(2H)-yl)-1-methyl-1H-indazol-6-yl)-3,6-diazabicyclo[3.1.1]heptane-6-carboxylate (48a)

To a stirred solution of 10c (300 mg, 1.513 mmol, 1 equiv) in dioxane (4 mL) were added 1-(6-bromo-1-methylindazol-3-yl)-1,3-diazinane-2,4-dione (488.96 mg, 1.513 mmol, 1 equiv), RuPhos Palladacycle Gen.3 (126.55 mg, 0.151 mmol, 0.1 equiv) and cesium carbonate (1479.01 mg, 4.539 mmol, 3 equiv) under nitrogen atmosphere. The resulting mixture was stirred for 3 hours at 100° C. under nitrogen atmosphere. The reaction was monitored by LCMS. Desired product could be detected by LCMS. The resulting mixture was diluted with saturated aqueous ammonium chloride (20 mL). The resulting mixture was extracted with ethyl acetate (2×15 mL). The combined organic layers were washed with brine (2×15 mL), dried over anhydrous sodium sulfate. After filtration, the filtrate was concentrated under reduced pressure. The residue was purified by silica gel column chromatography, eluted with 0% to 15% ethyl acetate in PE. Pure fractions were evaporated to dryness to afford 48a (200 mg, 28.51%) as a white solid. LCMS (ESI, m/z): [M+H]⁺ 441.2.

### Step B. 1-(6-(3,6-diazabicyclo[3.1.1]heptan-3-yl)-1-methyl-1H-indazol-3-yl)dihydropyrimidine-2,4(1H,3H)-dione (CI-48)

To a stirred solution of 48a (200 mg, 0.454 mmol, 1 equiv) in acetonitrile (4 mL) were added HCl (gas) in 1,4-dioxane (4 mL). The resulting mixture was stirred for 0.5 hours at 0° C. The reaction was monitored by LCMS. Desired product could be detected by LCMS. The residue was purified by reverse flash chromatography with the following conditions: column, C18 silica gel; mobile phase, acetonitrile in water, 5% to 50% gradient in 30 min; detector, UV 254 nm. Pure fractions were evaporated to dryness to afford CI-48 (90 mg, 52.41%) as a white solid. LCMS (ESI, m/z): [M+H]⁺ 341.0.

### Intermediate 49. 1-(6-{3,8-diazabicyclo[3.2.1]octan-3-yl]-1-methylindazol-3-yl)-1,3-diazinane-2,4-dione (CI-49)

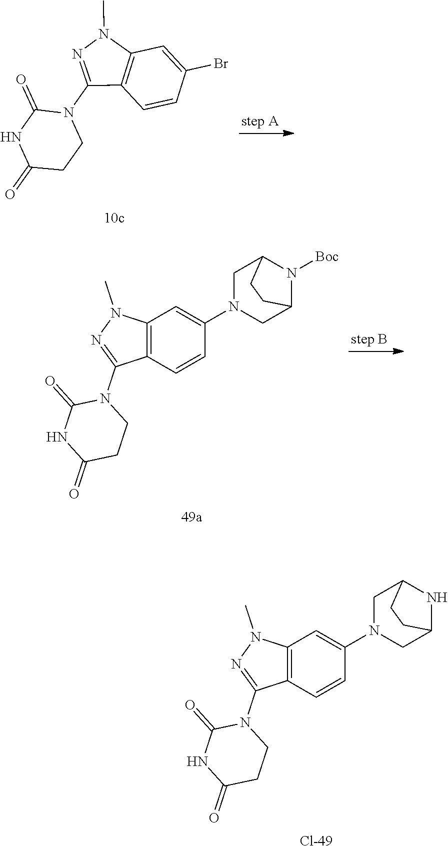

### Step A. tert-butyl 3-[3-(2,4-dioxo-1,3-diazinan-1-yl)-1-methylindazol-6-yl]-3,8-diazabicyclo[3.2.1]octane-8-carboxylate (49a)

A solution of 10c (500 mg, 1.55 mmol, 1 equiv), tert-butyl 3,8-diazabicyclo[3.2.1]octane-8-carboxylate (492.5 mg, 2.3 mmol, 1.5 equiv), RuPhos Pd G3 (262 mg, 0.3 mmol, 0.2 equiv), RuPhos (74 mg, 0.15 mmol, 0.1 equiv) and potassium phosphate tribasic (985.5 mg, 4.65 mmol, 3 equiv) in dioxane (5 mL) was stirred for overnight at 100° C. under argon atmosphere. Desired product could be detected by LCMS. The residue was purified by reversed-phase flash chromatography with the following conditions: column, C18 silica gel; mobile phase, acetonitrile in water (10 mmol/L ammonium bicarbonate), 10% to 50% gradient in 10 min; detector, UV 254 nm. Pure fractions were evaporated to dryness to afford 49a (300 mg, 54.21%) as a yellow solid. LCMS (ESI, m/z): [M+H]⁺ 455.23.

### Step B. 1-(6-{3,8-diazabicyclo[3.2.1]octan-3-yl}-1-methylindazol-3-yl)-1,3-diazinane-2,4-dione (CI-49)

A solution of 49a (120 mg, 0.264 mmol, 1 equiv) and HCl (g) in dioxane (1 mL, 32.913 mmol, 124.67 equiv) in dichloromethane (0.3 mL) was stirred for 30 min at room temperature under argon atmosphere. Desired product could be detected by LCMS. The residue was purified by reversed-phase flash chromatography with the following conditions: column, C18 silica gel; mobile phase, acetonitrile in water (10 mmol/L ammonium bicarbonate), 10% to 50% gradient in 10 min; detector, UV 254 nm. to afford CI-49 (45 mg, 48.09%) as a yellow solid. LCMS (ESI, m/z): [M+H]⁺ 355.18.

### Intermediate 50. 1-[6-(3,3-dimethylpiperazin-1-yl)-1-methylindazol-3-yl}-1,3-diazinane-2,4-dione (CI-50)

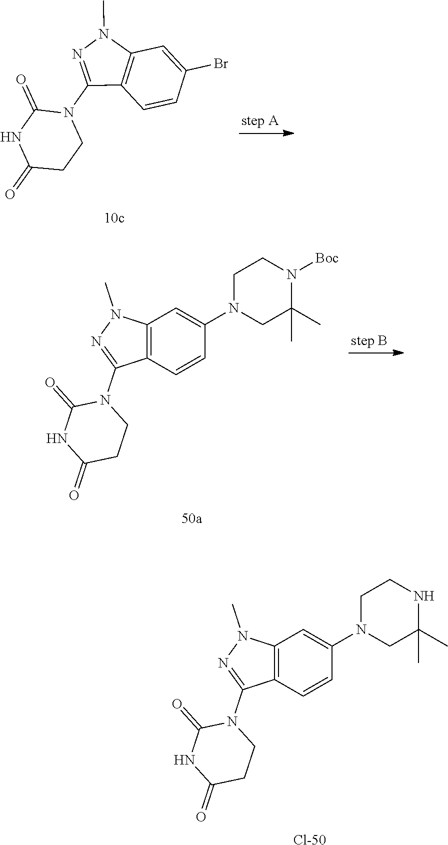

### Step A. tert-butyl 4-[3-(2,4-dioxo-1,3-diazinan-1-yl)-1-methylindazol-6-yl]-2,2-dimethylpiperazine-1-carboxylate (50a)

A mixture of 10c (500 mg, 1.547 mmol, 1 equiv), tert-butyl 2,2-dimethylpiperazine-1-carboxylate (497.39 mg, 2.321 mmol, 1.5 equiv), RuPhos Palladacycle Gen.3 (194.12 mg, 0.232 mmol, 0.15 equiv), RuPhos (108.30 mg, 0.232 mmol, 0.15 equiv) and potassium carbonate (641.52 mg, 4.641 mmol, 3 equiv) in dioxane (25 mL) was stirred for 2 h at 100° C. under nitrogen atmosphere. The reaction was monitored by LCMS. The mixture was allowed to cool down to room temperature. The resulting mixture was extracted with ethyl acetate (3×100 mL). The combined organic layers were washed with brine (3×10 mL), dried over anhydrous sodium sulfate. After filtration, the filtrate was concentrated under reduced pressure. The residue was purified by reversed-phase flash chromatography with the following conditions: column, C18 silica gel; mobile phase, acetonitrile in water (10 mmol/L ammonium bicarbonate), 10% to 50% gradient in 10 min; detector, UV 254 nm. This resulted in 50a (130 mg, 18.40%) as a yellow solid. LCMS (ESI, m/z): [M+H]⁺ 457.2.

### Step B. 1-[6-(3,3-dimethylpiperazin-1-yl)-1-methylindazol-3-yl]-1,3-diazinane-2,4-dione (CI-50)

A mixture of 50a (130 mg, 0.285 mmol, 1 equiv) and HCl (gas) in 1,4-dioxane (2.5 mL, 82.282 mmol, 288.97 equiv) in acetonitrile (5 mL) was stirred for 20 min at 0° C. under nitrogen atmosphere. The reaction was monitored by LCMS. The resulting mixture was concentrated under reduced pressure to afford CI-50 (130 mg, crude) as a white solid. LCMS (ESI, m/z): [M+H]⁺ 357.2.

### Intermediate 51L 1-(6-(2,5-diazabicyclo[2.2.1]heptan-2-yl)-1-methyl-1H-indazol-3-yl)dihydropyrimidine-2,4(1H,3H)-dione (CI-51)

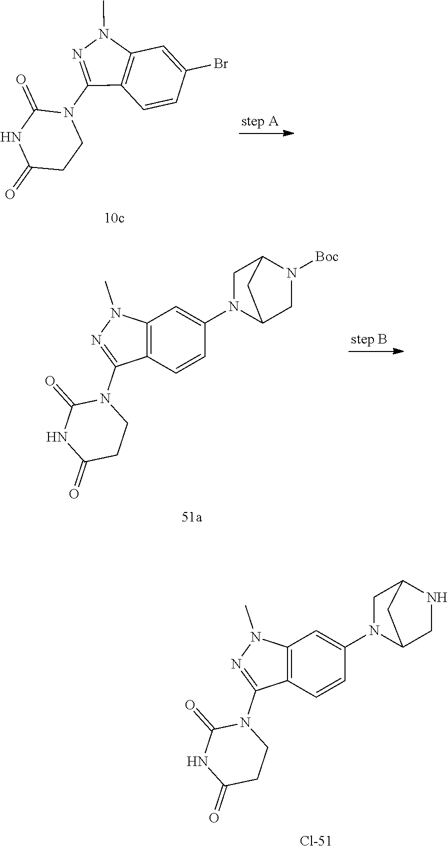

### Step A. tert-butyl 5-(3-(2,4-dioxotetrahydropyrimidin-1(2H)-yl)-1-methyl-1H-indazol-6-yl)-2,5-diazabicyclo[2.2.1]heptane-2-carboxylate (51a)

A solution of 10c (400 mg, 1.238 mmol, 1 equiv), tert-butyl 2,5-diazabicyclo[2.2.1]heptane-2-carboxylate (294.50 mg, 1.486 mmol, 1.2 equiv), Ruphos (57.76 mg, 0.124 mmol, 0.1 equiv), Pd(dppf)C₁₋₂ (90.57 mg, 0.124 mmol, 0.1 equiv) and potassium carbonate (342.14 mg, 2.476 mmol, 2 equiv) in 1,4-dioxane was stirred for 1 h at 90° C. under air atmosphere. Desired product could be detected by LCMS. The reaction was quenched with Water at room temperature. The resulting mixture was extracted with ethyl acetate (3×10 mL). The combined organic layers were washed with ethyl acetate (3×10 mL), dried over anhydrous sodium sulfate. After filtration, the filtrate was concentrated under reduced pressure. The residue was purified by silica gel column chromatography, eluted with dichloromethane/methanol (10:1) to afford 51a (500 mg, 91.70%) as a white solid. LCMS (ESI, m/z): [M+H]⁺ 441.2.

### Step B. 1-(6-(2,5-diazabicyclo[2.2.1]heptan-2-yl)-1-methyl-1H-indazol-3-yl)dihydropyrimidine-2,4(1H,3H)-dione (CI-51)

To a stirred solution of 51a (150 mg, 0.341 mmol, 1 equiv) and HCl (gas) in 1,4-dioxane (7.5 mL, 246.846 mmol, 724.91 equiv) in acetonitrile (15 mL) was stirred at 0° C. under air atmosphere for 1 h. The reaction was monitored by LCMS. The resulting mixture was concentrated under reduced pressure. The residue was diluted with dichloromethane and adjusted pH to 7-8 using triethylamine. The solvent was removed under reduced pressure to afford the crude product CI-51 (250 mg). The crude resulting was used in the next step directly without further purification. LCMS (ESI, m/z): [M+H]⁺ 341.0.

### Intermediate 52. 1-[6-(3-ethylpiperazin-1-yl)-1-methylindazol-3-yl]-1,3-diazinane-2,4-dione (CI-52)

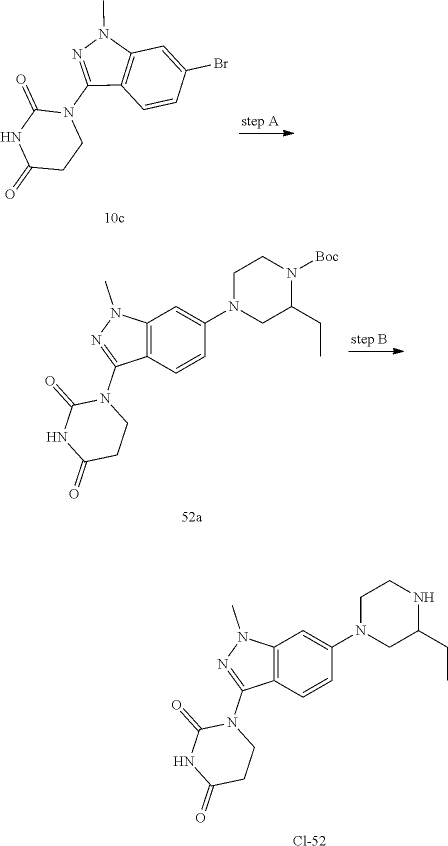

### Step A. tert-butyl 4-[3-(2,4-dioxo-1,3-diazinan-1-yl)-1-methylindazol-6-yl]-2-ethylpiperazine-1-carboxylate (52a)

To a stirred solution of 10c (600 mg, 1.857 mmol, 1 equiv) and RuPhos Palladacycle Gen.3 (155.29 mg, 0.186 mmol, 0.1 equiv) in dioxane (5 mL) were added RuPhos (86.64 mg, 0.186 mmol, 0.1 equiv) and cesium carbonate (1.81 g, 5.571 mmol, 3 equiv) in portions at room temperature under nitrogen atmosphere. The resulting mixture was stirred for 16 h at 100° C. under nitrogen atmosphere. Desired product could be detected by LCMS. The residue was purified by reversed-phase flash chromatography with the following conditions: column, C18 silica gel; mobile phase, acetonitrile in water (10 mmol/L ammonium bicarbonate), 10% to 52% gradient in 10 min; detector, UV 254 nm. This resulted in 52a (150 mg, 17.70%) as a white solid. LCMS (ESI, m/z): [M+H]⁺ 457.5.

### Step B. 1-[6-(3-ethylpiperazin-1-yl)-1-methylindazol-3-yl]-1,3-diazinane-2,4-dione (CI-52)

A solution of 52a (200 mg, 0.438 mmol, 1 equiv) and HCl (gas) in 1,4-dioxane (2.00 mL, 65.814 mmol, 150.26 equiv) in acetonitrile (2.00 mL, 38.040 mmol, 86.85 equiv) was stirred for 30 min at 0° C. under nitrogen atmosphere. Desired product could be detected by LCMS. The residue was purified by reversed-phase flash chromatography with the following conditions: column, C18 silica gel; mobile phase, acetonitrile in water (10 mmol/L ammonium bicarbonate), 10% to 30% gradient in 10 min; detector, UV 254 nm. This resulted in CI-52 (150 mg, 96.07%) as a white solid. LCMS (ESI, m/z): [M+H]⁺ 357.3.

### Intermediate 53. (3S)-3-(3-methyl-5-{1-[3-(methylamino)propyl]piperidin-4-yl}-2-oxo-1,3-benzodiazol-1-yl(CI-53)

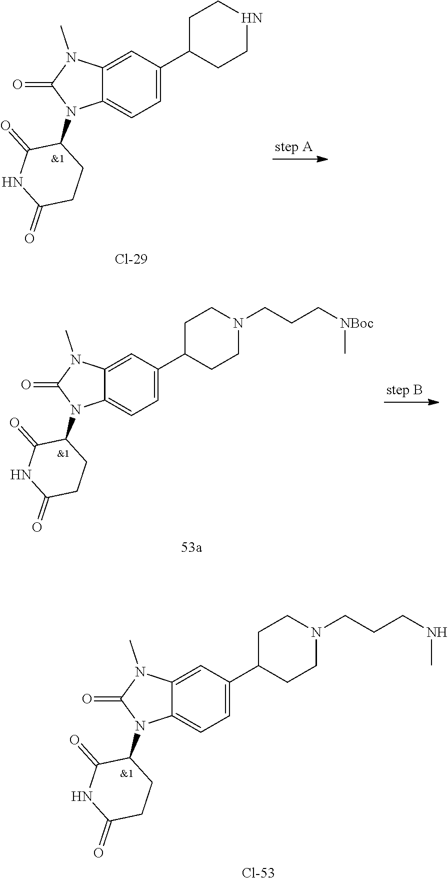

### Step A. tert-butyl N-[3-(4-{1-[(3S)-2,6-dioxopiperidin-3-yl]-3-methyl-2-oxo-1,3-benzodiazol-5-yl}piperidin-1-yl)propyl]-N-methylcarbamate (53a)

To a stirred mixture of CI-29 (130 mg, 0.380 mmol, 1 equiv) and tert-butyl N-methyl-N-(3-oxopropyl) carbamate (142.18 mg, 0.760 mmol, 2 equiv) in tetrahydrofuran (4 mL) and N,N-dimethylformamide (1 mL) was added sodium triacetoxyborohydride (241.40 mg, 1.140 mmol, 3 equiv) and acetic acid (11.40 mg, 0.190 mmol, 0.5 equiv) at room temperature under nitrogen atmosphere. The resulting mixture was stirred for overnight at room temperature under nitrogen atmosphere. Desired product could be detected by LCMS. The resulting mixture was diluted with water (50 mL). The resulting mixture was extracted with ethyl acetate (200 mL). The combined organic layers were washed with brine (3×20 mL), dried over anhydrous sodium sulfate. After filtration, the filtrate was concentrated under reduced pressure. The residue was purified by silica gel column chromatography, eluted with dichloromethane/methanol (10:1) to afford 53a (126 mg, 59.44%) as a white solid. LCMS (ESI, m/z): [M−H]⁺514.3.

### Step B. (3S)-3-(3-methyl-5-{1-[3-(methylamino)propyl]piperidin-4-yl}-2-oxo-1,3-benzodiazol-1-yl(CI-53)

To a stirred mixture of 53a (121 mg, 0.236 mmol, 1 equiv) in acetonitrile (5 mL) was added HCl (gas) in 1,4-dioxane (1 mL) dropwise at 0° C. under nitrogen atmosphere. The resulting mixture was stirred for 1 h at 0° C. under nitrogen atmosphere. Desired product could be detected by LCMS. The resulting mixture was concentrated under reduced pressure. The crude product was used in the next step directly without further purification. This resulted in CI-53 (121 mg, crude) as a light yellow solid. LCMS (ESI, m/z): [M−H]⁺414.2.

### Intermediate 54. 7-(17-[1-(1-ethoxyethyl)pyrazol-4-yl]-8-isopropoxy-[1,2,41 triazolo[1,5-cl pyrimidin-2-yl}amino)-1-benzofuran-4-sulfonyl chloride

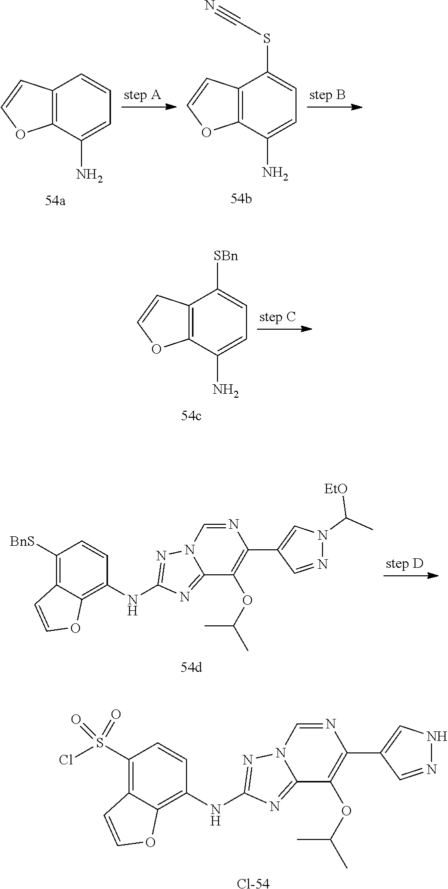

### Step A. [(7-amino-1-benzofuran-4-yl) sulfanyl]formonitrile (54b)

To a stirred solution of 54a (1 g, 7.510 mmol, 1 equiv) and ammonium thiocyanate (0.86 g, 11.265 mmol, 1.5 equiv) in methanol (10 mL) was added dipotassium 0-[(sulfonatoperoxy) sulfonyl]oxidoiodate (4.06 g, 15.020 mmol, 2 equiv) in portions at room temperature under nitrogen atmosphere. The resulting mixture was stirred for 3 days at room temperature under nitrogen atmosphere. Desired product could be detected by LCMS. The residue was purified by reversed-phase flash chromatography with the following conditions: column, C18 silica gel; mobile phase, acetonitrile in water (10 mmol/L ammonium bicarbonate), 10% to 50% gradient in 10 min; detector, UV 254 nm. This resulted in 54b (100 mg, 7.00%) as a black oil. LCMS (ESI, m/z): [M+H]191.2.

### Step B. 4-(benzylsulfanyl)-1-benzofuran-7-amine (54c)

To a stirred solution of 54b (200 mg, 1.051 mmol, 1 equiv) and (chloromethyl)benzene (133.09 mg, 1.051 mmol, 1 equiv) in tetrahydrofuran (2 mL) and water (1 mL) was added sodium hydroxide (126.16 mg, 3.153 mmol, 3 equiv) in portions at room temperature under nitrogen atmosphere. The resulting mixture was stirred for 1 h at 80° C. under nitrogen atmosphere. Desired product could be detected by LCMS. The residue was purified by silica gel column chromatography, eluted with petroleum ether/ethyl acetate (3:1) to afford 54c (55 mg, 20.49%) as a yellow solid. LCMS (ESI, m/z): [M+H]256.3.

### Step C. N-[4-(benzylsulfanyl)-1-benzofuran-7-yl]-7-[1-(1-ethoxyethyl) pyrazol-4-yl]-8-isopropoxy-[1,2,4]triazolo[1,5-c]pyrimidin-2-amine (54d)

To a stirred solution of 54c (50 mg, 0.196 mmol, 1 equiv), CI-1 (77.40 mg, 0.196 mmol, 1 equiv) and Pd₂(dba)₃ (35.86 mg, 0.039 mmol, 0.2 equiv) in dioxane (2 mL) were added SPhos (16.08 mg, 0.039 mmol, 0.2 equiv) and cesium carbonate (191.40 mg, 0.588 mmol, 3 equiv) in portions at room temperature under nitrogen atmosphere. The resulting mixture was stirred for 1 h at 90° C. under nitrogen atmosphere. Desired product could be detected by LCMS. The residue was purified by Prep-TLC (petroleum ether/ethyl acetate3:1) to afford 54d (60 mg, 53.79%) as a yellow solid. LCMS (ESI, m/z): [M+H]⁺ 570.7.

### Step D. 7-({7-[1-(1-ethoxyethyl) pyrazol-4-yl]-8-isopropoxy-[1,2,4]triazolo[1,5-c]pyrimidin-2-yl}amino)-1-benzofuran-4-sulfonyl chloride (CI-54)

To a stirred solution of sulfonyl chloride (44.98 mg, 0.704 mmol, 4 equiv) in dichloromethane (2 mL) were added 54d (100 mg, 0.176 mmol, 1 equiv), acetic acid (42.17 mg, 0.704 mmol, 4 equiv) and water (12.65 mg, 0.704 mmol, 4 equiv) in portions at 0° C. under nitrogen atmosphere. The resulting mixture was stirred for 0.5h at 0° C. under nitrogen atmosphere. The reaction was monitored by TLC (3:1). The resulting mixture was diluted with dichloromethane (2 mL). The resulting mixture was washed with 3×3 mL of water. The resulting mixture was concentrated under vacuum. This resulted in CI-54 (35 mg) as a crude product which was used in the next step directly without further purification. LCMS (ESI, m/z): [M+H]⁺ 546.1.

### Intermediate 55. 4-f[8-isopropoxy-7-(1H-pyrazol-4-yl)-[1,2,4]triazolo[1,5-c]pyrimidin-2-yl]amino}benzenesulfonyl chloride (CI-55)

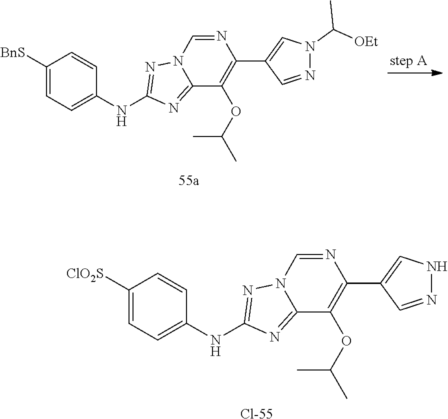

### Step A. 4-{[8-isopropoxy-7-(1H-pyrazol-4-yl)-[1,2,4]triazolo[1,5-c]pyrimidin-2-yl]amino}benzenesulfonyl chloride (CI-55)

A solution of 55a (150 mg, 0.283 mmol, 1 equiv), water (20.41 mg, 1.132 mmol, 4 equiv) and sulfonyl chloride (152.88 mg, 1.132 mmol, 4 equiv) in dichloromethane (1 mL) was stirred for 2 h at room temperature under argon atmosphere. Desired product could be detected by LCMS. The resulting mixture was extracted with dichloromethane (2×20 mL). The combined organic layers were washed with dichloromethane (2×20 mL), dried over anhydrous sodium sulfate. After filtration, the filtrate was concentrated under reduced pressure. The crude product/resulting mixture was used in the next step directly without further purification.

### Intermediate 56. 3-((4-((7-(1-(1-ethoxyethyl)-1H-pyrazol-4-yl)-8-isopropoxy-11,2,4]triazolo[1,5-c]pyrimidin-2-yl)amino)-3-fluoro-N,2-dimethylphenyl)sulfonamido)propyl methanesulfonate (CI-56)

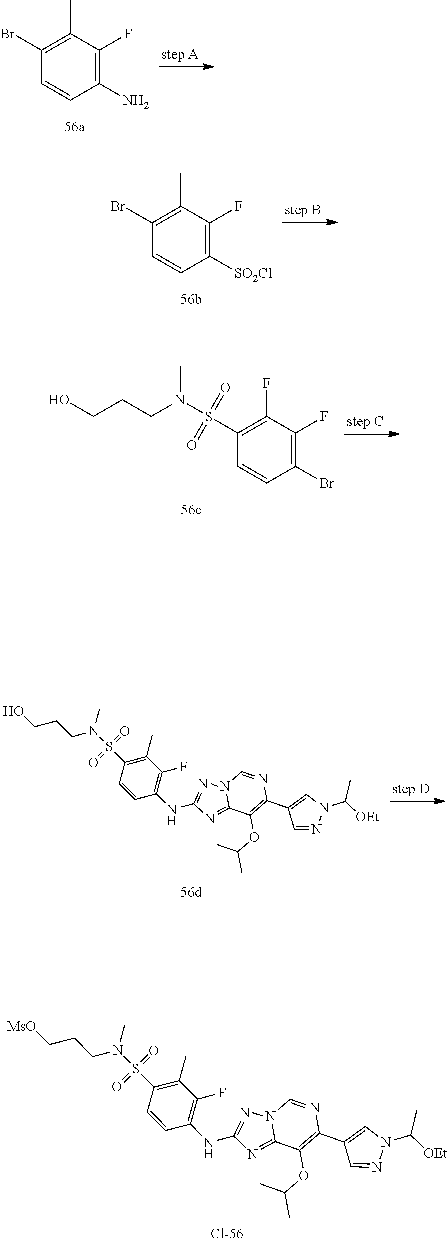

### Step A. 4-bromo-2,5-difluoro-N-(3-hydroxypropyl)-N-methylbenzenesulfonamide (56b)

To a stirred mixture of 56a (800 mg, 3.921 mmol, 1 equiv) and concentrated HCl (5718.04 mg, 156.840 mmol, 40 equiv) in water (100 mL) were added sodium nitrite (324.62 mg, 4.705 mmol, 1.2 equiv) at 0° C. under N2. The resulting mixture was stirred for 1 h at 0° C. To the above mixture was added thionyl chloride (10261.10 mg, 86.262 mmol, 22 equiv) and cuprous chloride (38.82 mg, 0.392 mmol, 0.1 equiv) in water (10 mL) dropwise over 10 min at 0° C. The resulting mixture was stirred for additional 1 h at 0° C. Desired product could be detected by LCMS. The mixture was concentrated under vacuum. The resulting mixture was diluted with water (50 mL) and extracted with ethyl acetate (3×100 mL). The combined organic layers were dried over anhydrous sodium sulfate and concentrated under reduced pressure. The residue was purified by reversed-phase Combi-Flash, eluted with acetonitrile:water (0.5% ammonium bicarbonate)=5%-60%. This resulted in 56b (600 mg, 48.43%) as a yellow oil. LCMS (ESI, m/z): [M+H]344.0.

### Step B. 4-bromo-3-fluoro-N-(3-hydroxypropyl)-N,2-dimethylbenzenesulfonamide (56c)

To a stirred mixture of 3-(methylamino) propan-1-ol (600 mg, 6.731 mmol, 1 equiv) and 56b (1935.40 mg, 6.731 mmol, 1 equiv) in dichloromethane (5 mL) were added trimethylamine (2043.43 mg, 20.193 mmol, 3 equiv) at 0° C. The resulting mixture was stirred for 1 h at 25° C. Desired product could be detected by LCMS. The mixture was concentrated under vacuum. The resulting mixture was diluted with water (5 mL) and extracted with ethyl acetate (3×10 mL). The combined organic layers were dried over anhydrous sodium sulfate and concentrated under reduced pressure. The residue was purified by reversed-phase Combi-Flash, eluted with acetonitrile:water (0.5% ammonium bicarbonate)=5%-60%. This resulted in 56c (700 mg, 27.21%) as a yellow solid. LCMS (ESI, m/z): [M+H]340.0.

### Step C. 4-((7-(1-(1-ethoxyethyl)-1H-pyrazol-4-yl)-8-isopropoxy-[1,2,4]triazolo[1,5-c]pyrimidin-2-yl)amino)-3-fluoro-N-(3-hydroxypropyl)-N,2-dimethylbenzenesulfonamide (56d)

To a stirred mixture of 56c (200 mg, 0.588 mmol, 1 equiv) and 1g (194.81 mg, 0.588 mmol, 1 equiv) and cesium carbonate (574.62 mg, 1.764 mmol, 3 equiv) in dioxane (10 mL) were added Xantphos Pd G3 (111.50 mg, 0.118 mmol, 0.2 equiv) and Xantphos (68.03 mg, 0.118 mmol, 0.2 equiv) at 25° C. under N₂. The resulting mixture was stirred for 2 h at 90° C. Desired product could be detected by LCMS. The mixture was concentrated under vacuum. The resulting mixture was diluted with water (50 mL) and extracted with ethyl acetate (3×100 mL). The combined organic layers were dried over anhydrous sodium sulfate and concentrated under reduced pressure. The residue was purified by reversed-phase Combi-Flash, eluted with acetonitrile:water (0.5% ammonium bicarbonate)=5%-60%. This resulted in 56d (130 mg, 31.82%) as a yellow solid. LCMS (ESI, m/z): [M+H]591.2.

### Step D. 3-((4-((7-(1-(1-ethoxyethyl)-1H-pyrazol-4-yl)-8-isopropoxy-[1,2,4]triazolo[1,5-c]pyrimidin-2-yl)amino)-3-fluoro-N,2-dimethylphenyl)sulfonamido)propyl methanesulfonate (CI-56)

To a stirred mixture of 56d (100 mg, 0.169 mmol, 1 equiv) and trimethylamine (68.53 mg, 0.676 mmol, 4 equiv) in dichloromethane (10 mL) were added methanesulfonic anhydride (58.98 mg, 0.338 mmol, 2 equiv) at 0° C. The resulting mixture was stirred for 1 h at 25° C. Desired product could be detected by LCMS. The mixture was concentrated under vacuum. The resulting mixture was diluted with water (5 mL) and extracted with ethyl acetate (3×10 mL). The combined organic layers were dried over anhydrous sodium sulfate and concentrated under reduced pressure. The residue was purified by reversed-phase Combi-Flash, eluted with acetonitrile:water (0.5% ammonium bicarbonate)=5%-60%. This resulted in CI-56 (80 mg, 52.99%) as a yellow solid. LCMS (ESI, m/z): [M+H]⁺ 669.2.

### Intermediate 57. 3-[N-methyl4-({7-[1-(1-ethoxyethyl) pyrazol-4-yl}-8-isopropoxy-11,2,41triazolo[1,5-c]pyrimidin-2-yl}amino)-2,3-difluorobenzenesulfonamide propyl methanesulfonate (CI-57)

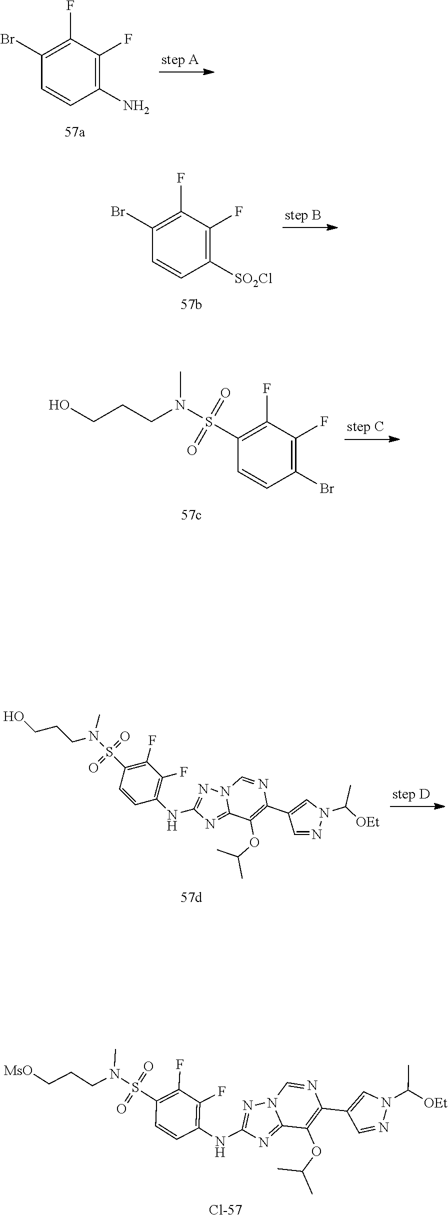

### Step A. 4-bromo-2,3-difluorobenzenesulfonyl chloride (57b)

To a stirred mixture of cuprous chloride (0.05 g, 0.481 mmol, 0.1 equiv) in water (5 mL) was added thionyl chloride (0.80 g, 6.731 mmol, 1.4 equiv) dropwise at 0° C. under nitrogen atmosphere, and the resulting mixture was stirred for 1 h at 0° C. under nitrogen atmosphere as solution A.

The solution of 57a (1 g, 4.808 mmol, 1 equiv) in water (5 mL) was treated with sodium nitrite (0.38 g, 5.529 mmol, 1.15 equiv) in water (5 mL) for 1 h at 0° C. under nitrogen atmosphere as solution B. The slurry from B was added dropwise to solution A while keeping the temperature of 0° C. and this mixture stirred for 2h. Desired product could be detected by LCMS. The resulting mixture was diluted with water (100 mL). The resulting mixture was extracted with ethyl acetate (400 mL). The combined organic layers were washed with brine (200 mL), dried over anhydrous sodium sulfate. After filtration, the filtrate was concentrated under reduced pressure. The residue was purified by silica gel column chromatography, eluted with petroleum ether/ethyl acetate(1:1) to afford 57b (332 mg, 21.32%) as a light yellow oil. LCMS (ESI, m/z): [M+H]⁺ 291.0.

### Step B. 4-bromo-2,3-difluoro-N-(3-hydroxypropyl)-N-methylbenzenesulfonamide (57c)

To a stirred solution of 57b (340 mg, 1.166 mmol, 1 equiv) and 3-(methylamino) propan-1-ol (103.97 mg, 1.166 mmol, 1 equiv) in dichloromethane (10 mL) was added trimethylamine (354.10 mg, 3.498 mmol, 3 equiv) dropwise at room temperature under nitrogen atmosphere. The resulting mixture was stirred for 2 h at room temperature under nitrogen atmosphere. Desired product could be detected by LCMS. The resulting mixture was concentrated under reduced pressure. The residue was purified by reversed-phase flash chromatography with the following conditions: column, C18 silica gel; mobile phase, acetonitrile in water (10 mmol/L ammonium bicarbonate), 30% to 50% gradient in 10 min; detector, UV 254 nm. This resulted in 57c (300 mg, 67.26%) as a light yellow solid. LCMS (ESI, m/z): [M+H]⁺ 344.0.

### Step C. 4-((7-(1-(1-ethoxyethyl)-1H-pyrazol-4-yl)-8-isopropoxy-[1,2,4]triazolo[1,5-c]pyrimidin-2-yl)amino)-3-fluoro-N-(3-hydroxypropyl)-N,2-dimethylbenzenesulfonamide (57d)

To a stirred mixture of 57c (300 mg, 0.872 mmol, 1 equiv) and 1g (288.85 mg, 0.872 mmol, 1 equiv) in dioxane (5 mL) was added Xanthphos Pd G3 (124.00 mg, 0.131 mmol, 0.15 equiv) XantPhos (100.87 mg, 0.174 mmol, 0.2 equiv) and cesium carbonate (852.01 mg, 2.616 mmol, 3 equiv) at room temperature under nitrogen atmosphere. The resulting mixture was stirred for 2 h at 90° C. under nitrogen atmosphere. Desired product could be detected by LCMS. The resulting mixture was diluted with water (100 mL). The resulting mixture was extracted with ethyl acetate (200 mL). The combined organic layers were washed with brine (100 mL), dried over anhydrous sodium sulfate. After filtration, the filtrate was concentrated under reduced pressure. The residue was purified by reversed-phase flash chromatography with the following conditions: column, C18 silica gel; mobile phase, acetonitrile in water (10 mmol/L ammonium bicarbonate), 50% to 70% gradient in 10 min; detector, UV 254 nm. This resulted in 57d (358 mg, 63.77%) as a brown yellow solid. LCMS (ESI, m/z): [M+H]⁺ 595.0.

### Step D. 3-[N-methyl4-({7-[1-(1-ethoxyethyl) pyrazol-4-yl]-8-isopropoxy-[1,2,4]triazolo[1,5-c]pyrimidin-2-yl}amino)-2,3-difluorobenzenesulfonamido]propyl methanesulfonate (CI-57)

To a stirred mixture of 57d (230 mg, 0.387 mmol, 1 equiv) and trimethylamine (234.84 mg, 2.322 mmol, 6 equiv) in dichloromethane (5 mL) was added methanesulfonyl methanesulfonate (134.75 mg, 0.774 mmol, 2 equiv) at room temperature under nitrogen atmosphere. The resulting mixture was stirred for 1 h at room temperature under nitrogen atmosphere. Desired product could be detected by LCMS. The resulting mixture was diluted with water (100 mL). The resulting mixture was extracted with ethyl acetate (200 mL). The combined organic layers were washed with brine (50 mL), dried over anhydrous sodium sulfate. After filtration, the filtrate was concentrated under reduced pressure. The residue was purified by silica gel column chromatography, eluted with petroleum ether/ethyl acetate(5:1) to afford CI-57 (241 mg, 83.36%) as a light yellow solid. LCMS (ESI, m/z): [M+H]⁺ 673.0.

### Intermediate 58. 3-fluoro-4-((8-isopropoxy-7-(1H-pyrazol-4-yl)-[1,2,4]triazolo[1,5-c]pyrimidin-2-yl)amino)-5-methylbenzenesulfonyl chloride (CI-58)

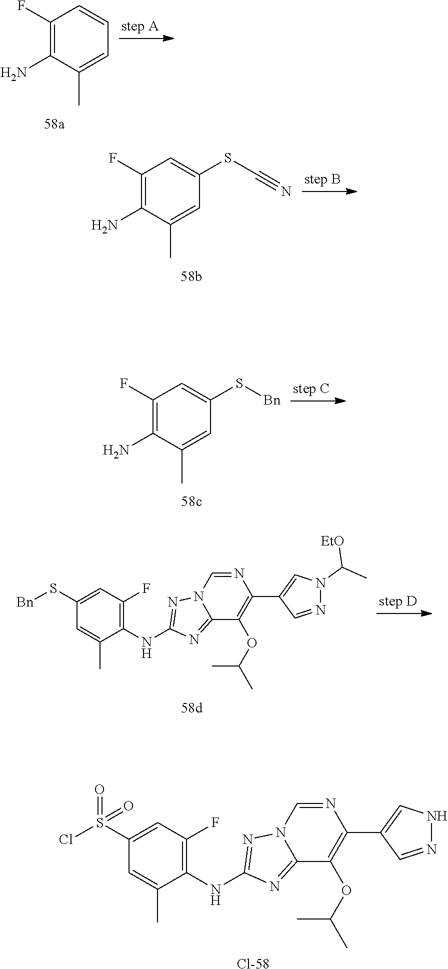

### Step A. [(4-amino-3-fluoro-5-methylphenyl)sulfanyl]formonitrile (58b)

To a stirred mixture of 58a (2 g, 15.981 mmol, 1 equiv) and trimethylisocyanate (3.17 g, 31.962 mmol, 2 equiv) in dimethyl sulfoxide (20 mL) was added sulfur (1.02 g, 31.962 mmol, 2 equiv) in portions at room temperature. The resulting mixture was stirred for overnight at 90° C. The reaction was monitored by LCMS. The resulting mixture was extracted with ethyl acetate (3×50 mL). The combined organic layers were washed with brine (3×10 mL), dried over anhydrous sodium sulfate. After filtration, the filtrate was concentrated under reduced pressure. The residue was purified by silica gel column chromatography, eluted with petroleum ether/ethyl acetate(1:1) to afford 58b (2 g, 68.68%) as a white solid. LCMS (ESI, m/z): [M+H]⁺ 183.0

### Step B. 4-(benzylsulfanyl)-2-fluoro-6-methylaniline) (58c)

To a stirred mixture of 58b (490 mg, 2.689 mmol, 1 equiv) and benzyl chloride (340.38 mg, 2.689 mmol, 1 equiv) in tetrahydrofuran (10 mL) and water (5 mL) was added sodium hydroxide (322.66 mg, 8.067 mmol, 3 equiv) in portions at room temperature. The resulting mixture was stirred for 2 h at 80° C. The reaction was monitored by TLC. The resulting mixture was concentrated under reduced pressure. The resulting mixture was extracted with ethyl acetate (3×50 mL), dried over anhydrous sodium sulfate. After filtration, the filtrate was concentrated under reduced pressure. The residue was purified by silica gel column chromatography, eluted with petroleum ether/ethyl acetate(5:1) to afford 58c as a white solid. LCMS (ESI, m/z): [M+H]⁺ 248.0

### Step C. N-[4-(benzylsulfanyl)-2-fluoro-6-methylphenyl]-7-[1-(1-ethoxyethyl)pyrazol-4-yl]-8-isopropoxy-[1,2,4]triazolo[1,5-c]pyrimidin-2-amine (58d)

To a stirred mixture of 58c (383.55 mg, 0.971 mmol, 1.2 equiv) and CI-1 (500.85 mg, 0.872 mmol, 1 equiv) in dioxane (3 mL) were added Pd₂(dba)₃ (148.10 mg, 0.162 mmol, 0.2 equiv), Sphos (66.40 mg, 0.162 mmol, 0.2 equiv) and cesium carbonate (790.41 mg, 2.427 mmol, 3 equiv) in portions at room temperature under argon atmosphere. The resulting mixture was stirred for 1 h at 100° C. under argon atmosphere. The reaction was monitored by TLC. The resulting mixture was concentrated under reduced pressure. The residue was purified by silica gel column chromatography, eluted with petroleum ether/ethyl acetate(3:1) to afford 58d (160 mg, 35.23%) as a white solid. LCMS (ESI, m/z): [M+H]⁺ 562.2

### Step D. 3-fluoro-4-((8-isopropoxy-7-(1H-pyrazol-4-yl)-[1,2,4]triazolo[1,5-c]pyrimidin-2-yl)amino)-5-methylbenzenesulfonyl chloride (CI-58)

To a stirred mixture of 58d (148 mg, 0.263 mmol, 1 equiv) and water (18.99 mg, 1.052 mmol, 4 equiv) in dichloromethane (1.5 mL) were added acetic acid (63.29 mg, 1.052 mmol, 4 equiv) and sulfonyl chloride (142.25 mg, 1.052 mmol, 4 equiv) in portions at 0° C. The resulting mixture was stirred for 30 min at 0° C. The reaction was monitored by LCMS. The resulting mixture was washed with 3×10 mL of dichloromethane. The resulting mixture was concentrated under reduced pressure. This resulted in CI-58 (130 mg) as crude product which was used in the next step directly without further purification. LCMS (ESI, m/z): [M+H]⁺ 466.0

### Intermediate 59. 3-((4-((7-(1-(1-ethoxyethyl)-1H-pyrazol-4-yl)-8-isopropoxy-[1,2,4]triazolo[1,5-cl]pyrimidin-2-yl)amino)-3,5-difluoro-N-methylphenyl)sulfonamido)propyl methanesulfonate (CI-59)

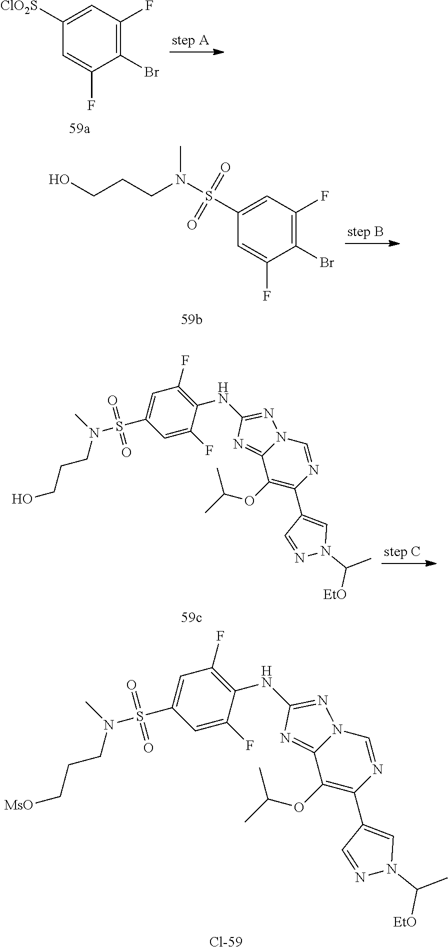

### Step A. 4-bromo-3,5-difluoro-N-(3-hydroxypropyl)-N-methylbenzenesulfonamide (59b)

To a stirred mixture of 59a (2 g, 6.861 mmol, 1 equiv) and trimethylamine (2.08 g, 20.583 mmol, 3 equiv) in acetonitrile (20 mL) was added 3-(methylamino)propan-1-ol (0.61 g, 6.861 mmol, 1 equiv) in portions at 0° C. The resulting mixture was stirred for 1 h at room temperature. The reaction was monitored by TLC. The resulting mixture was concentrated under reduced pressure. The residue was purified by silica gel column chromatography, eluted with petroleum ether/ethyl acetate(1:2) to afford 59b (2 g, 84.69%) as a white solid. LCMS (ESI, m/z): [M+H]⁺ 343.9

### Step B. 4-{7-[1-(1-ethoxyethyl)pyrazol-4-yl]-8-isopropoxy-[1,2,4]triazolo[1,5-c]pyrimidin-2-yl}-3,5-difluoro-N-(3-hydroxypropyl)-N-methylbenzenesulfonamide (59c)

To a stirred mixture of 59b (600 mg, 1.743 mmol, 1 equiv) and 1g (866.55 mg, 2.615 mmol, 1.5 equiv) in dioxane (6 mL) were added Xantphos Pd G3 (247.99 mg, 0.261 mmol, 0.15 equiv), Xantphos (151.31 mg, 0.261 mmol, 0.15 equiv) and cesium carbonate (1704.02 mg, 5.229 mmol, 3 equiv) in portions at room temperature under argon atmosphere. The resulting mixture was stirred for 1 h at 90° C. under argon atmosphere. The reaction was monitored by LCMS. The resulting mixture was concentrated under reduced pressure. The residue was purified by silica gel column chromatography, eluted with petroleum ether/ethyl acetate(1:1) to afford 59c (500 mg, 49.48%) as a white solid. LCMS (ESI, m/z): [M+H]⁺ 595.2

### Step C. 3-((4-((7-(1-(1-ethoxyethyl)-1H-pyrazol-4-yl)-8-isopropoxy-[1,2,4]triazolo[1,5-c]pyrimidin-2-yl)amino)-3,5-difluoro-N-methylphenyl)sulfonamido)propyl methanesulfonate (CI-59)

To a stirred mixture of 59c (400 mg, 0.673 mmol, 1 equiv) and trimethylamine (408.42 mg, 4.038 mmol, 6 equiv) in dichloromethane (0.5 mL) was added methanesulfonyl chloride (231.14 mg, 2.019 mmol, 3 equiv) in portions at 0° C. The resulting mixture was stirred for 1 h at 0° C. The reaction was monitored by TLC. The resulting mixture was concentrated under reduced pressure. The resulted in CI-59 (500 mg) as crude product which was used in the next step directly without further purification. LCMS (ESI, m/z): [M+H]⁺ 673.2

### Intermediate 60. 3-[N-methyl4-({7-[1-(1-ethoxyethyl)pyrazol-4-yl}-8-isopropoxy-[1,2,4]triazolo[1,5-c]pyrimidin-2-yl}amino)-5-fluoro-2-methylbenzenesulfonamido]propyl methanesulfonate (CI-60)

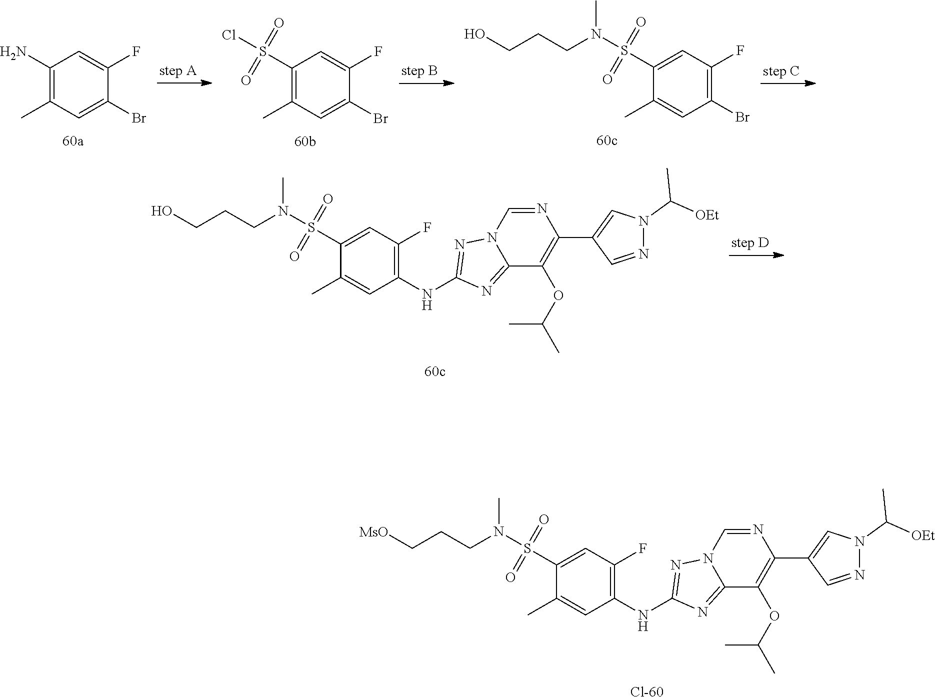

### Step A. 4-bromo-5-fluoro-2-methylbenzenesulfonyl chloride (60b)

To a stirred mixture of 60a (3 g, 14.703 mmol, 1 equiv) and HCl (54.00 mL, 647.961 mmol, 44.07 equiv) in water (48.00 mL, 2664.478 mmol, 181.22 equiv) was added sodium nitrite (1136.15 mg, 16.467 mmol, 1.12 equiv) and water (48.00 mL, 2664.478 mmol, 181.22 equiv) in portions at 0° C. The resulting mixture was stirred for 1 h at 0° C. as solution A. To a stirred mixture of cuprous chloride (160.11 mg, 1.617 mmol, 0.11 equiv) in water (48.00 mL, 2664.478 mmol, 181.22 equiv) was added thionyl chloride (8.40 mL, 115.860 mmol, 7.88 equiv) in portions at 0° C. The resulting mixture was stirred for 1 h at 0° C. as solution B. To the above solution B was added solution A in portions at 0° C. The resulting mixture was stirred for 2 h at 0° C. Desired product could be detected by LCMS. The resulting mixture was extracted with acetic ester. The combined organic layers were dried over anhydrous sodium sulfate. After filtration, the filtrate was concentrated under reduced pressure. The residue was purified by silica gel column chromatography, eluted with petroleum ether/dichloromethane (30:1) to afford 60b (1.6 g, crude) as a colorless oil. LCMS (ESI, m/z): [M+H]⁺ 286.9.

### Step B. 4-bromo-5-fluoro-N-(3-hydroxypropyl)-N,2-dimethylbenzenesulfonamide (60c)

To a stirred mixture of 60b (1.5 g, 5.217 mmol, 1 equiv) in dichloromethane (15 mL) was added trimethylamine (1583.73 mg, 15.651 mmol, 3 equiv) and 3-(methylamino)propan-1-ol (697.53 mg, 7.825 mmol, 1.5 equiv). The resulting mixture was stirred for 1 h at 25° C. Desired product could be detected by LCMS. The resulting mixture was concentrated under reduced pressure. The residue was purified by silica gel column chromatography, eluted with petroleum ether/ethyl acetate (1:1) to afford 60c (1.6 g, crude) as a white solid. LCMS (ESI, m/z): [M+H]⁺ 340.0.

### Step C. 4-({7-[1-(1-ethoxyethyl)pyrazol-4-yl]-8-isopropoxy-[1,2,4]triazolo[1,5-c]pyrimidin-2-yl}amino)-5-fluoro-N-(3-hydroxypropyl)-N,2-dimethylbenzenesulfonamide (60d)

To a stirred mixture of 60c (369.59 mg, 1.086 mmol, 1.2 equiv) and 1g (300 mg, 0.905 mmol, 1.00 equiv) in dioxane (4 mL) was added RuPhos palladacycle Gen.3 (151.44 mg, 0.181 mmol, 0.2 equiv) and Xantphos (104.77 mg, 0.181 mmol, 0.2 equiv) and cesium carbonate (884.90 mg, 2.715 mmol, 3 equiv). The resulting mixture was stirred for 1 h at 90° C. Desired product could be detected by LCMS. The resulting mixture was concentrated under reduced pressure. The residue was purified by silica gel column chromatography, eluted with petroleum ether/ethyl acetate (1:10) to afford 60d (300 mg, 52.96%) as a yellow solid. LCMS (ESI, m/z): [M+H]⁺ 591.2.

### Step D. 3-[N-methyl4-({7-[1-(1-ethoxyethyl)pyrazol-4-yl]-8-isopropoxy-[1,2,4]triazolo[1,5-c]pyrimidin-2-yl}amino)-5-fluoro-2-methylbenzenesulfonamido]propyl methanesulfonate (CI-60)

To a stirred mixture of 60d (250 mg, 0.423 mmol, 1 equiv) in dichloromethane (3 mL) was added methanesulfonic anhydride (147.45 mg, 0.846 mmol, 2 equiv) and trimethylamine (171.32 mg, 1.692 mmol, 4 equiv). The resulting mixture was stirred for 0.5 h at 0° C. Desired product could be detected by LCMS.

The resulting mixture was concentrated under reduced pressure. The residue was purified by silica gel column chromatography, eluted with petroleum ether/ethyl acetate (1:1) to afford CI-60 (260 mg, 82.67%) as a white solid. LCMS (ESI, m/z): [M+H]⁺ 669.2.

### Intermediate 61. 3-((4-((7-(1-(1-ethoxyethyl)-1H-pyrazol-4-yl)-8-isopropoxy-[1,2,4]triazolo[1,5-c]pyrimidin-2-yl)amino)-2,5-difluoro-N-methylphenyl)sulfonamido)propyl methanesulfonate (CI-61)

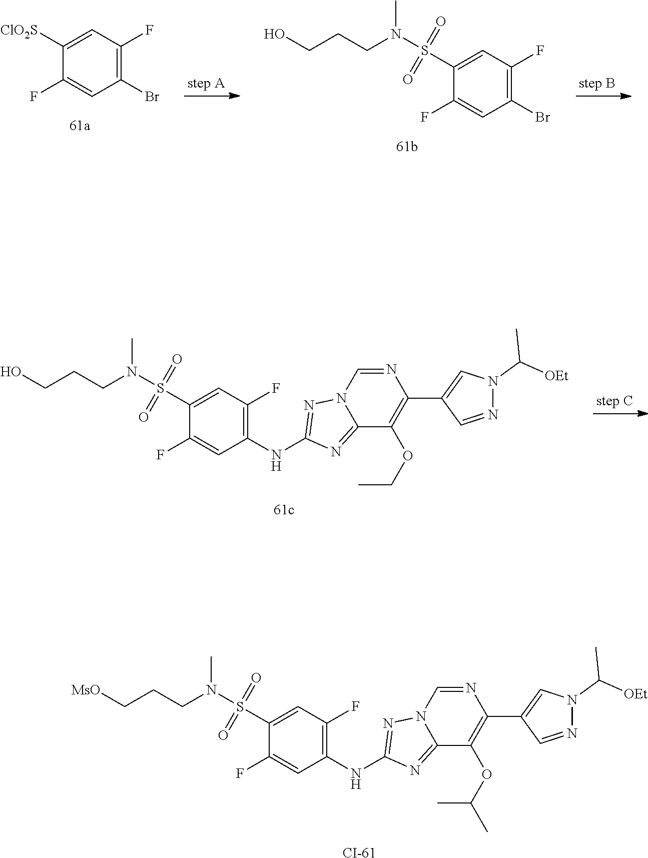 [SMILES: O=S(=O)(Cl)c1cc(F)c(Br)cc1F ; CN(CCCO)S(=O)(=O)c1cc(F)c(Br)cc1F ; CCOc1c(-c2cnn(C(C)OCC)c2)ncn2nc(Nc3cc(F)c(S(=O)(=O)N(C)CCCO)cc3F)nc12 ; CCOC(C)n1cc(-c2ncn3nc(Nc4cc(F)c(S(=O)(=O)N(C)CCCOS(C)(=O)=O)cc4F)nc3c2OC(C)C)cn1 ; "CI-61"]

### Step A. 4-bromo-2-fluoro-3-methylbenzenesulfonyl chloride (61b)

To a stirred mixture of 3-(methylamino) propan-1-ol (152.90 mg, 1.715 mmol, 1 equiv) and 61a (500 mg, 1.715 mmol, 1.00 equiv) in dichloromethane (5 mL) were added trimethylamine (520.74 mg, 5.145 mmol, 3 equiv) at 0° C. The resulting mixture was stirred for 1 h at 25° C. Desired product could be detected by LCMS. The mixture was concentrated under vacuum. The resulting mixture was diluted with water (5 mL) and extracted with ethyl acetate (3×10 mL). The combined organic layers were dried over anhydrous sodium sulfate and concentrated under reduced pressure. The residue was purified by reversed-phase Combi-Flash, eluted with acetonitrile:water (0.5% ammonium bicarbonate)=5%-60%.This resulted in 61b (600 mg, 87.40%) as a yellow solid. LCMS (ESI, m/z): [M+H]286.9.

### Step B. 4-((8-ethoxy-7-(1-(1-ethoxyethyl)-1H-pyrazol-4-yl)-[1,2,4]triazolo[1,5-c]pyrimidin-2-yl)amino)-2,5-difluoro-N-(3-hydroxypropyl)-N-methylbenzenesulfonamide (61c)

To a stirred mixture of 61b (300 mg, 0.872 mmol, 1 equiv) and 1g (288.85 mg, 0.872 mmol, 1 equiv) and cesium carbonate (568.01 mg, 1.744 mmol, 2 equiv) in dioxane (8 mL) were added Ruphos (81.35 mg, 0.174 mmol, 0.2 equiv) and Ruphos Pd G3 (135.41 mg, 0.174 mmol, 0.2 equiv) at 25° C. under N2. The resulting mixture was stirred for 2 h at 90° C. Desired product could be detected by LCMS. The mixture was concentrated under vacuum. The resulting mixture was diluted with water (50 mL) and extracted with ethyl acetate (3×100 mL). The combined organic layers were dried over anhydrous sodium sulfate and concentrated under reduced pressure. The residue was purified by reversed-phase Combi-Flash, eluted with acetonitrile:water (0.5% ammonium bicarbonate)=5%-60%.This resulted in 61c (200 mg, 33.18%) as a yellow solid. LCMS (ESI, m/z): [M+H]581.2.

### Step C. 3-((4-((7-(1-(1-ethoxyethyl)-1H-pyrazol-4-yl)-8-isopropoxy-[1,2,4]triazolo[1,5-c]pyrimidin-2-yl)amino)-2,5-difluoro-N-methylphenyl)sulfonamido)propyl methanesulfonate (CI-61)

To a stirred mixture of 61c (300 mg, 0.505 mmol, 1 equiv) and trimethylamine (204.21 mg, 2.020 mmol, 4 equiv) in dichloromethane (5 mL) were added methanesulfonic anhydride (175.76 mg, 1.010 mmol, 2 equiv) at 0° C. The resulting mixture was stirred for 1 h at 25° C. Desired product could be detected by LCMS. The resulting mixture was diluted with water (5 mL) and extracted with ethyl acetate (3×10 mL). The combined organic layers were dried over anhydrous sodium sulfate and concentrated under reduced pressure. The residue was purified by reversed-phase Combi-Flash, eluted with acetonitrile:water (0.5% ammonium bicarbonate) 5%-60%.This resulted in CI-61 (230 mg, 54.89%) as a yellow solid. LCMS (ESI, m/z): [M+H]673.2.

### Intermediate 62. 5-{18-isopropoxy-7-(1H-pyrazol-4-yl)-[1,2,41triazolo[1,5-c]pyrimidin-2-yl}amino}isoquinoline-8-sulfonyl chloride (CI-62)

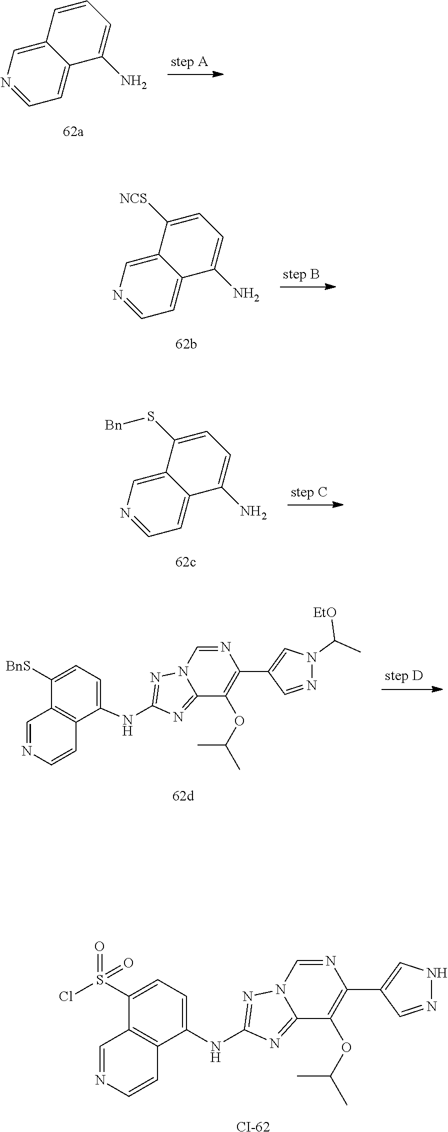

### Step A. [(5-aminoisoquinolin-8-yl) sulfanyl]formonitrile (62b)

To a stirred solution of 62a (2 g, 13.872 mmol, 1 equiv) and ammonium thiocyanate (1.16 g, 15.259 mmol, 1.1 equiv) in methanol (40 mL) was added Potassium persulfate (5.62 g, 20.808 mmol, 1.5 equiv) at room temperature under nitrogen atmosphere. The resulting mixture was stirred for 15 h at room temperature under nitrogen atmosphere. 60% Desired product could be detected by LCMS. The resulting mixture was filtered, the filter cake was washed with methanol (2×40 mL). The filtrate was concentrated under reduced pressure. The residue was purified by reversed-phase flash chromatography with the following conditions: column, C18 silica gel; mobile phase, methanol in water, 10% to 60% gradient in 60 min; detector, UV 254 nm. This resulted in 62b (1 g, 35.82%) as a light brown solid. LCMS (ESI, m/z): [M+H]⁺ 202.0.

### Step B. 8-(benzylsulfanyl)isoquinolin-5-amine (62c)

To a stirred solution of 62b (900 mg, 4.472 mmol, 1 equiv) and benzyl chloride (622.68 mg, 4.919 mmol, 1.1 equiv) in tetrahydrofuran (12 mL)/water (6 mL) was added sodium hydroxide (536.61 mg, 13.416 mmol, 3 equiv) at room temperature. The resulting mixture was stirred for 1 h at 80° C. under nitrogen atmosphere. 90% Desired product could be detected by LCMS. The mixture was allowed to cool down to room temperature. The resulting mixture was diluted with brine (20 mL). The combined organic layers were washed with brine (2×20 mL), dried over anhydrous sodium sulfate. After filtration, the filtrate was concentrated under reduced pressure. The residue was purified by reversed-phase flash chromatography with the following conditions: column, C18 silica gel; mobile phase, acetonitrile in water (0.1% formic acid), 10% to 60% gradient in 40 min; detector, UV 254 nm. This resulted in 62c (900 mg, 75.56%) as a yellow solid. LCMS (ESI, m/z): [M+H]⁺ 267.0.

### Step C. 8-(benzylsulfanyl)-N-{7-[1-(1-ethoxyethyl)pyrazol-4-yl]-8-isopropoxy-[1,2,4]triazolo[1,5-c]pyrimidin-2-yl}isoquinolin-5-amine (62d)

To a stirred solution of 62c (300 mg, 1.126 mmol, 1 equiv) and CI-1 (534.22 mg, 1.351 mmol, 1.2 equiv) in dioxane (3 mL) were added Pd₂(dba)₃ (1031.38 mg, 1.126 mmol, 1 equiv) and cesium carbonate (73.39 mg, 0.225 mmol, 0.2 equiv)in portions at room temperature under nitrogen atmosphere. The resulting mixture was stirred for 1 h at 90° C. under nitrogen atmosphere. Desired product could be detected by LCMS.

The resulting mixture was extracted with ethyl acetate (3×10 mL). The combined organic layers were washed with brine (3×10 mL), dried over anhydrous sodium sulfate. After filtration, the filtrate was concentrated under reduced pressure. The resulting mixture was concentrated under reduced pressure. The residue was purified by silica gel column chromatography, eluted with petroleum ether/ethyl acetate(1:1) to afford 62d (400 mg, 51.1%) as a yellow solid. LCMS (ESI, m/z): [M+H]581.2.

### Step D. 5-{[8-isopropoxy-7-(1H-pyrazol-4-yl)-[1,2,4]triazolo[1,5-c]pyrimidin-2-yl]amino}isoquinoline-8-sulfonyl chloride (CI-62)

A solution of 62d (80 mg, 0.138 mmol, 1 equiv) in dichloromethane (2 mL) was treated with acetic acid (33.09 mg, 0.552 mmol, 4 equiv) for 5 min at 0° C. under nitrogen atmosphere followed by the addition of sulfonyl chloride (74.37 mg, 0.552 mmol, 4 equiv) dropwise at 0° C. The resulting mixture was stirred for 30 min at 0° C. under nitrogen atmosphere. Desired product could be detected by LCMS. The resulting mixture was extracted with dichloromethane (5×10 mL). The combined organic layers were washed with rine (2×10 mL), dried over anhydrous sodium sulfate. After filtration, the filtrate was concentrated under reduced pressure. The resulting mixture was concentrated under reduced pressure to afford CI-62 (120 mg, crude) as a yellow solid. LCMS (ESI, m/z): [M+H]⁺ 486.0.

### Intermediate 63. 3-13-methyl-5-[(3S)-3-methyl-4-[3-(methylamino) propyl]ninerazin-1-yl}-2-oxo-1,3-benzodiazol-1-yl}piperidine-2,6-dione (CI-63)

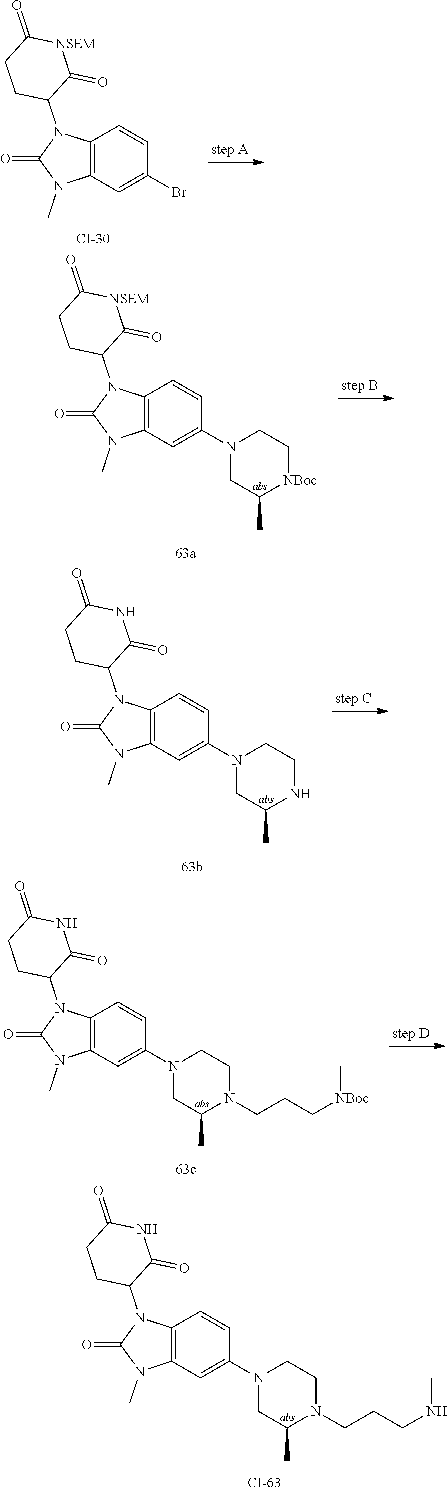

### Step A. tert-butyl (2S)-4-[1-(2,6-dioxo-1-{[2-(trimethylsilyl) ethoxy]methyl}piperidin-3-yl)-3-methyl-2-oxo-1,3-benzodiazol-5-yl]-2-methylpiperazine-1-carboxylate (63a)

To a stirred mixture of CI-30 (500 mg, 1.067 mmol, 1.00 equiv) and tert-butyl N-methyl-N-[(2S)-1-(methylamino) propan-2-yl]carbamate (431.87 mg, 2.134 mmol, 2 equiv) in dioxane (5 mL) were added RuPhos Pd G4 (181.54 mg, 0.213 mmol, 0.2 equiv), Ruphos (1.00 mg, 0.002 mmol, 0.1 equiv) and cesium carbonate (695.57 mg, 2.134 mmol, 2 equiv) in portions at room temperature under nitrogen atmosphere. The solution was stirred at 100° C. for 2 h. Desired product could be detected by LCMS. The resulting mixture was extracted with ethyl acetate (3×15 mL). The combined organic layers were washed with brine (3×10 mL), dried over anhydrous sodium sulfate. The resulting mixture was concentrated under reduced pressure. The residue was purified by silica gel column chromatography, eluted with petroleum ether/ethyl acetate(1:3) to afford 63a (300 mg, 47.82%) as a brown yellow solid. LCMS (ESI, m/z): [M+H]⁺ 588.0.

### Step B. 3-{3-methyl-5-[(3S)-3-methylpiperazin-1-yl]-2-oxo-1,3-benzodiazol-1-yl}piperidine-2,6-dione (63b)

A solution of 63a (200 mg, 0.340 mmol, 1 equiv) and trifluoroacetic acid (1.5 mL) in dichloromethane (2 mL) was stirred for 2 h at 0° C. under air atmosphere. Desired product could be detected by LCMS. The resulting mixture was concentrated under reduced pressure to afford 63b (150 mg, crude) as a yellow solid. LCMS (ESI, m/z): [M+H]⁺ 488.0

### Step C. tert-butyl N-{3-[(2S)-4-[1-(2,6-dioxopiperidin-3-yl)-3-methyl-2-oxo-1,3-benzodiazol-5-yl]-2-methylpiperazin-1-yl]propyl}-N-methylcarbamate (63c)

To a stirred solution of 63b (200 mg, 0.560 mmol, 1 equiv) and tert-butyl N-methyl-N-(3-oxopropyl) carbamate (224 mg, 1.120 mmol, 2 equiv) in N,N-dimethylformamide (2 mL) was added acetic acid (100.84 mg, 1.68 mmol, 3 equiv) in portions at room temperature under air atmosphere. The resulting mixture was stirred for 1 h at 50° C. under air atmosphere. To the above mixture was added sodium triacetoxyborohydride (35.630 mg, 0.168 mmol, 3 equiv). The resulting mixture was stirred for additional 1 h at 50° C. Desired product could be detected by LCMS. The residue was purified by reversed-phase flash chromatography with the following conditions: column, C18 silica gel; mobile phase, acetonitrile in water (10 mmol/L ammonium bicarbonate), 30% to 70% gradient in 30 min to afford 63c (70 mg, 35.00%) as a white solid. LCMS (ESI, m/z): [M+H]⁺ 529.3.

### Step D. 3-{3-methyl-5-[(3S)-3-methyl-4-[3-(methylamino) propyl]piperazin-1-yl]-2-oxo-1,3-benzodiazol-1-yl}piperidine-2,6-dione (CI-63)

To a stirred solution of 63c (70 mg, 0.133 mmol, 1 equiv) in acetonitrile (5 mL) was added 4M HCl (gas) in 1,4-dioxane (5 mL) dropwise at 0° C. The resulting mixture was stirred for 1 h at 0° C. Desired product could be detected by LCMS. The resulting mixture was concentrated under reduced pressure to afford CI-63 (50 mg, 61.09%) as a white solid. LCMS (ESI, m/z): [M+H]⁺ 429.2.

### Intermediate 64. 3-{3-methyl-5-[(3R)-3-methyl-4-[3-(methylamino)propyl]piperazin-1-yl}-2-oxo-1,3-benzodiazol-1-yl}piperidine-2,6-dione (CI-64)

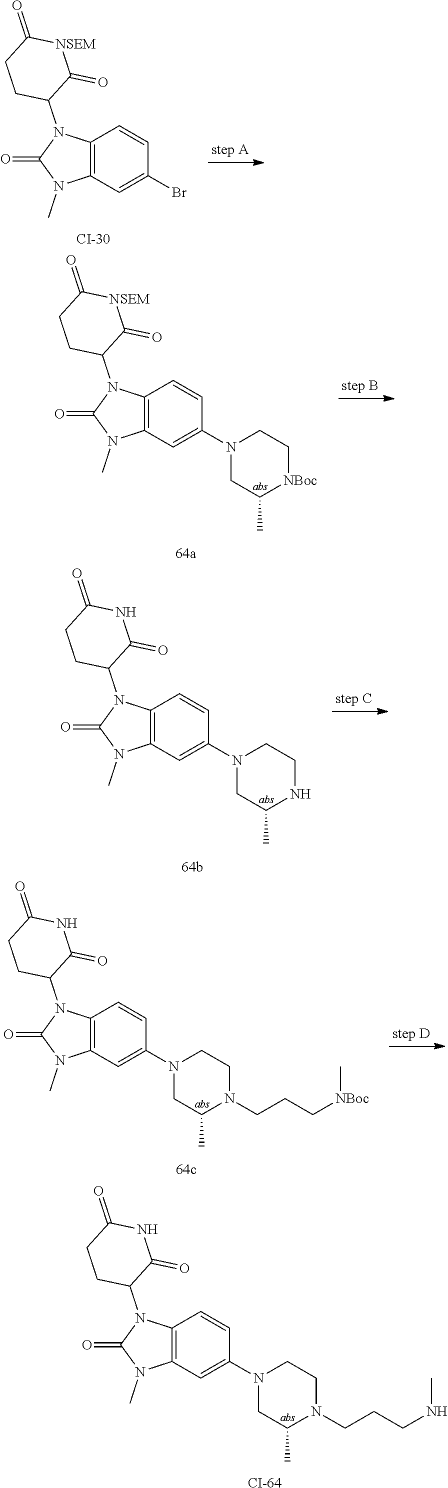

Step A. tert-butyl (2R)-4-[1-(2,6-dioxo-1-{[2-(trimethylsilyl)ethoxy]methyl}piperidin-3-yl)-3-methyl-2-oxo-1,3-benzodiazol-5-yl]-2-methylpiperazine-1-carboxylate (64a)

To a stirred mixture of CI-30 (500 mg, 1.067 mmol, 1.00 equiv) and tert-butyl N-methyl-N-[(2S)-1-(methylamino) propan-2-yl]carbamate (431.87 mg, 2.134 mmol, 2 equiv) in dioxane (5 mL) were added RuPhos Pd G4 (181.54 mg, 0.213 mmol, 0.2 equiv), Ruphos (1.00 mg, 0.002 mmol, 0.1 equiv) and cesium carbonate (695.57 mg, 2.134 mmol, 2 equiv) in portions at room temperature under nitrogen atmosphere. The solution was stirred at 100° C. for 2 h. Desired product could be detected by LCMS. The resulting mixture was extracted with ethyl acetate (3×15 mL). The combined organic layers were washed with brine (3×10 mL), dried over anhydrous sodium sulfate. The resulting mixture was concentrated under reduced pressure. The residue was purified by silica gel column chromatography, eluted with petroleum ether/ethyl acetate(1:3) to afford 64a (300 mg, 47.82%) as a brown yellow solid. LCMS (ESI, m/z): [M+H]⁺ 588.0.

### Step B. 3-{3-methyl-5-[(3R)-3-methylpiperazin-1-yl]-2-oxo-1,3-benzodiazol-1-yl}piperidine-2,6-dione (64b)

A solution of 64a (200 mg, 0.340 mmol, 1 equiv) and trifluoroacetic acid (1.5 mL) in dichloromethane (2 mL) was stirred for 2 h at 0° C. under air atmosphere. Desired product could be detected by LCMS. The resulting mixture was concentrated under reduced pressure to afford 64b (150 mg, crude) as a yellow solid. LCMS (ESI, m/z): [M+H]⁺ 488.0.

### Step C. tert-butyl N-{3-[(2R)-4-[1-(2,6-dioxopiperidin-3-yl)-3-methyl-2-oxo-1,3-benzodiazol-5-yl]-2-methylpiperazin-1-yl]propyl}-N-methylcarbamate (64c)

To a stirred solution of 64b (200 mg, 0.560 mmol, 1 equiv) and tert-butyl N-methyl-N-(3-oxopropyl) carbamate (224 mg, 1.120 mmol, 2 equiv) in N,N-dimethylformamide (2 mL) was added acetic acid (100.84 mg, 1.68 mmol, 3 equiv) in portions at room temperature under air atmosphere. The resulting mixture was stirred for 1 h at 50° C. under air atmosphere. To the above mixture was added sodium triacetoxyborohydride (35.630 mg, 0.168 mmol, 3 equiv). The resulting mixture was stirred for additional 1 h at 50° C. Desired product could be detected by LCMS. The residue was purified by reversed-phase flash chromatography with the following conditions: column, C18 silica gel; mobile phase, acetonitrile in water (10 mmol/L ammonium bicarbonate), 30% to 70% gradient in 30 min to afford 64c (70 mg, 35.00%) as a white solid. LCMS (ESI, m/z): [M+H]⁺ 529.3.

### Step D. 3-{3-methyl-5-[(3R)-3-methyl-4-[3-(methylamino) propyl]piperazin-1-yl]-2-oxo-1,3-benzodiazol-1-yl}piperidine-2,6-dione (CI-64)

To a stirred solution of 64c (70 mg, 0.133 mmol, 1 equiv) in acetonitrile (5 mL) was added 4M HCl (gas) in 1,4-dioxane (5 mL) dropwise at 0° C. The resulting mixture was stirred for 1 h at 0° C. Desired product could be detected by LCMS. The resulting mixture was concentrated under reduced pressure to afford CI-64 (50 mg, 61.09%) as a white solid. LCMS (ESI, m/z): [M+H]⁺ 429.2.

### Intermediate 65. 3-15-[(3S)-3-isopropyl-4-[3-(methylamino)propyl]piperazin-1-yl]-3-methyl-2-oxo-1,3-benzodiazol-1-yl}piperidine-2,6-dione (CI-65)

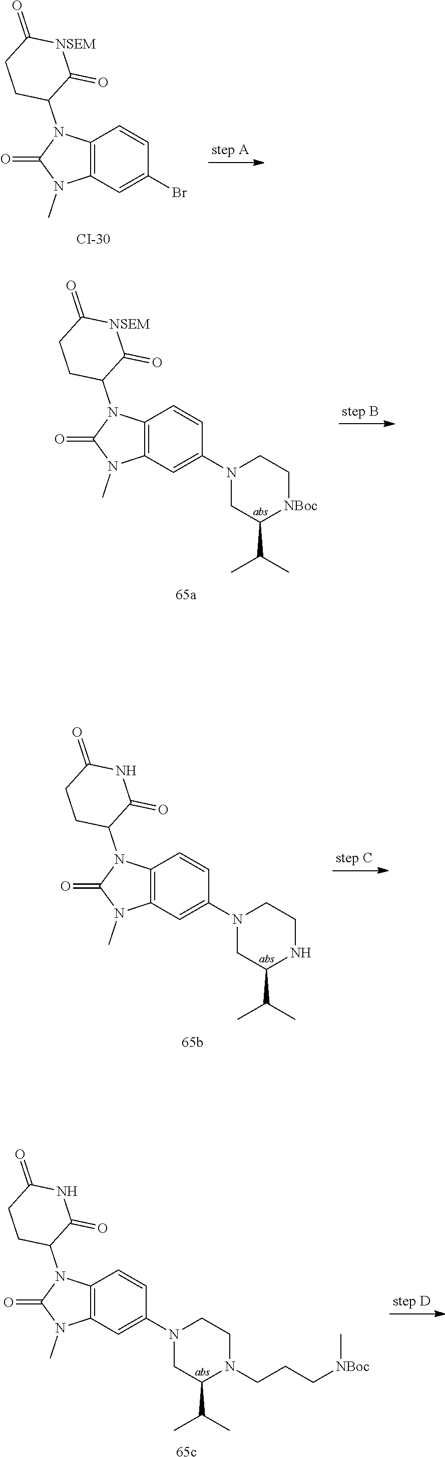

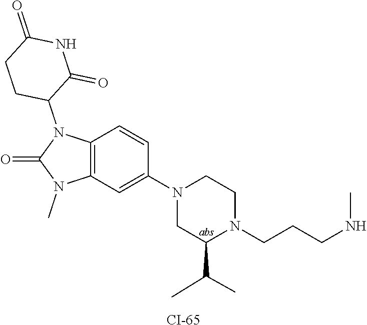

### Step A. tert-butyl (2S)-4-[1-(2,6-dioxo-1-{[2-(trimethylsilyl)ethoxy]methyl}piperidin-3-yl)-3-methyl-2-oxo-1,3-benzodiazol-5-yl]-2-isopropylpiperazine-1-carboxylate (65a)

A solution of CI-30 (400 mg, 0.854 mmol, 1 equiv), tert-butyl (2S)-2-isopropylpiperazine-1-carboxylate (292.47 mg, 1.281 mmol, 1.5 equiv), Ruphos Pd G4 (78.20 mg, 0.085 mmol, 0.1 equiv), Ruphos (39.85 mg, 0.085 mmol, 0.1 equiv) and cesium carbonate (556.45 mg, 1.708 mmol, 2 equiv) in dioxane was stirred for 1 h at 110° C. under air atmosphere. Desired product could be detected by LCMS. The reaction was quenched with Water at room temperature. The aqueous layer was extracted with ethyl acetate (3×10 mL). The resulting mixture was concentrated under reduced pressure. The residue was purified by Prep-TLC (dichloromethane/methanol 10:1) to afford 65a (350 mg, 66.55%) as a white solid. LCMS (ESI, m/z): [M+H]⁺ 616.3.

### Step B. 3-{5-[(3S)-3-isopropylpiperazin-1-yl]-3-methyl-2-oxo-1,3-benzodiazol-1-yl}piperidine-2,6-dione (65b)

A solution of tert-butyl 65a (240 mg, 0.390 mmol, 1 equiv) and trifluoroacetic acid (0.43 mL, 5.850 mmol, 15 equiv) in dichloromethane (4 mL) was stirred for 1 h at room temperature under air atmosphere. Desired product could be detected by LCMS. The resulting mixture was concentrated under reduced pressure. The crude product/resulting mixture was used in the next step directly without further purification. This resulted in 65b (150 mg, 99.85%) as a white oil. LCMS (ESI, m/z): [M+H]⁺ 386.2.

### Step C. tert-butyl N-{3-[(2S)-4-[1-(2,6-dioxopiperidin-3-yl)-3-methyl-2-oxo-1,3-benzodiazol-5-yl]-2-isopropylpiperazin-1-yl]propyl}-N-methylcarbamate (65c)

A solution of 65b (160 mg, 0.415 mmol, 1 equiv), tert-butyl N-methyl-N-(3-oxopropyl)carbamate (155.44 mg, 0.830 mmol, 2 equiv), sodium triacetoxyborohydride (263.92 mg, 1.245 mmol, 3 equiv) and acetic acid (237.85 uL, 4.150 mmol, 10 equiv) in N,N-dimethylformamide (3 mL) was stirred for 1 h at room temperature under air atmosphere. Desired product could be detected by LCMS. The reaction was quenched with Water at room temperature. The aqueous layer was extracted with ethyl acetate (3×5 mL). The resulting mixture was concentrated under reduced pressure. The residue was purified by silica gel column chromatography, eluted with petroleum ether/ethyl acetate(5:1) to afford 65c (120 mg, 51.93%) as a white solid. LCMS (ESI, m/z): [M+H]⁺ 557.3.

### Step D. 3-{5-[(3S)-3-isopropyl-4-[3-(methylamino)propyl]piperazin-1-yl]-3-methyl-2-oxo-1,3-benzodiazol-1-yl}piperidine-2,6-dione (CI-65)

A solution of 65c (120 mg, 0.216 mmol, 1 equiv) and HCl(gas)in 1,4-dioxane (0.5 mL, 2.000 mmol, 9.28 equiv) in dichloromethane (2 mL) was stirred for 1 h at room temperature under air atmosphere. Desired product could be detected by LCMS. The resulting mixture was concentrated under reduced pressure. This resulted in CI-65 (95 mg, 96.53%) as a white solid. LCMS (ESI, m/z): [M+H]⁺ 457.3.

### Intermediate 66. 3-{5-[(3R)-3-isopropyl-4-[3-(methylamino)propyl]piperazin-1-yl]-3-methyl-2-oxo-1,3-benzodiazol-1-yl}piperidine-2,6-dione (CI-66)

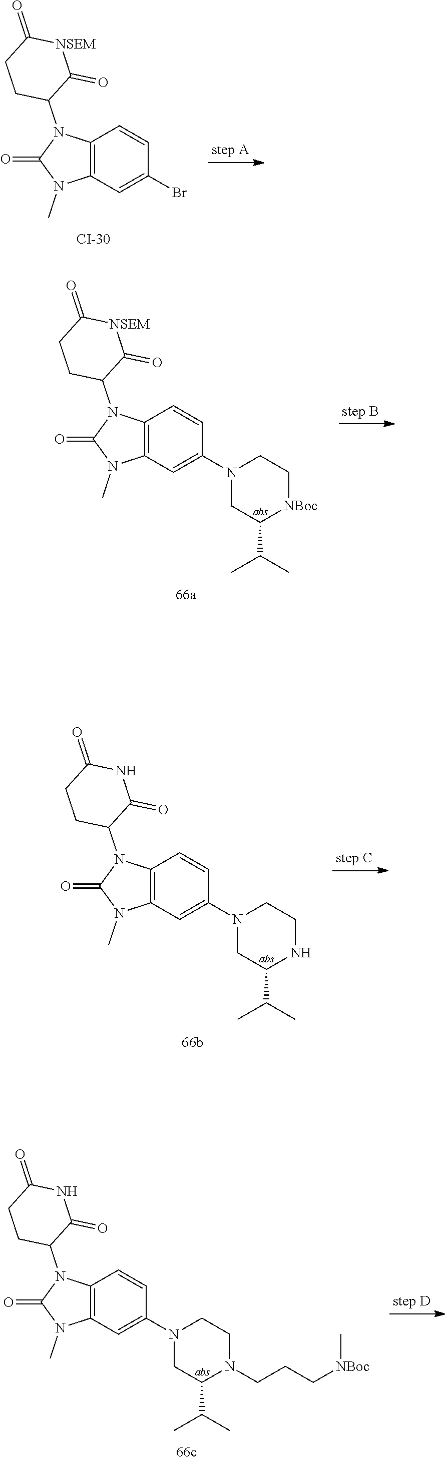

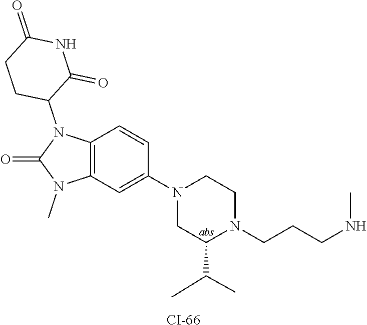

### Step A. tert-butyl (2R)-4-[1-(2,6-dioxo-1-{[2-(trimethylsilyl)ethoxy]methyl}piperidin-3-yl)-3-methyl-2-oxo-1,3-benzodiazol-5-yl]-2-isopropylpiperazine-1-carboxylate (66a)

A solution of CI-30 (400 mg, 0.854 mmol, 1 equiv), tert-butyl (2S)-2-isopropylpiperazine-1-carboxylate (292.47 mg, 1.281 mmol, 1.5 equiv), Ruphos Pd G4 (78.20 mg, 0.085 mmol, 0.1 equiv), Ruphos (39.85 mg, 0.085 mmol, 0.1 equiv) and cesium carbonate (556.45 mg, 1.708 mmol, 2 equiv) in dioxane was stirred for 1 h at 110° C. under air atmosphere. Desired product could be detected by LCMS. The reaction was quenched with Water at room temperature. The aqueous layer was extracted with ethyl acetate (3×10 mL). The resulting mixture was concentrated under reduced pressure. The residue was purified by Prep-TLC (dichloromethane/methanol 10:1) to afford 66a (350 mg, 66.55%) as a white solid. LCMS (ESI, m/z): [M+H]⁺ 616.3.

### Step B. 3-{5-[(3R)-3-isopropylpiperazin-1-yl]-3-methyl-2-oxo-1,3-benzodiazol-1-yl}piperidine-2,6-dione (66b)

A solution of tert-butyl 66a (240 mg, 0.390 mmol, 1 equiv) and trifluoroacetic acid (0.43 mL, 5.850 mmol, 15 equiv) in dichloromethane (4 mL) was stirred for 1 h at room temperature under air atmosphere. Desired product could be detected by LCMS. The resulting mixture was concentrated under reduced pressure. The crude product/resulting mixture was used in the next step directly without further purification. This resulted in 66b (150 mg, 99.85%) as a white oil. LCMS (ESI, m/z): [M+H]⁺ 386.2.

### Step C. tert-butyl N-{3-[(2R)-4-[1-(2,6-dioxopiperidin-3-yl)-3-methyl-2-oxo-1,3-benzodiazol-5-yl]-2-isopropylpiperazin-1-yl]propyl}-N-methylcarbamate (66c)

A solution of 66b (160 mg, 0.415 mmol, 1 equiv), tert-butyl N-methyl-N-(3-oxopropyl)carbamate (155.44 mg, 0.830 mmol, 2 equiv), sodium triacetoxyborohydride (263.92 mg, 1.245 mmol, 3 equiv) and acetic acid (237.85 uL, 4.150 mmol, 10 equiv) in N,N-dimethylformamide (3 mL) was stirred for 1 h at room temperature under air atmosphere. Desired product could be detected by LCMS. The reaction was quenched with Water at room temperature. The aqueous layer was extracted with ethyl acetate (3×5 mL). The resulting mixture was concentrated under reduced pressure. The residue was purified by silica gel column chromatography, eluted with petroleum ether/ethyl acetate(5:1) to afford 66c (120 mg, 51.93%) as a white solid. LCMS (ESI, m/z): [M+H]⁺ 557.3.

### Step D. 3-{5-[(3S)-3-isopropyl-4-[3-(methylamino)propyl]piperazin-1-yl]-3-methyl-2-oxo-1,3-benzodiazol-1-yl}piperidine-2,6-dione (CI-66)

A solution of 66c (120 mg, 0.216 mmol, 1 equiv) and HCl(gas)in 1,4-dioxane (0.5 mL, 2.000 mmol, 9.28 equiv) in dichloromethane (2 mL) was stirred for 1 h at room temperature under air atmosphere. Desired product could be detected by LCMS. The resulting mixture was concentrated under reduced pressure. This resulted in CI-66 (95 mg, 96.53%) as a white solid. LCMS (ESI, m/z): [M+H]⁺ 457.3.

### Intermediate 67. 3-(3-methyl-5-(4-(3-(methylamino)propyl)piperazin-1-yl)-2-oxo-2,3-dihydro-1H-benzo[d]imidazol-1-yl)piperidine-2,6-dione (CI-67)

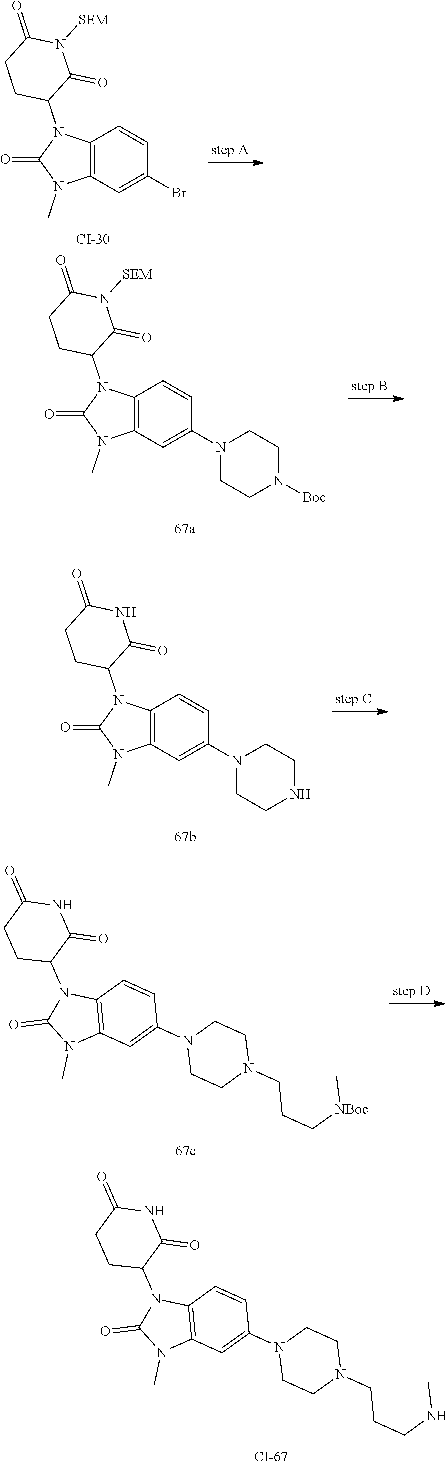

### Step A. tert-butyl 4-[1-(2,6-dioxo-1-{[2-(trimethylsilyl)ethoxy]methyl}piperidin-3-yl)-3-methyl-2-oxo-1,3-benzodiazol-5-yl]piperazine-1-carboxylate (67a)

To a stirred mixture of CI-30 (400 mg, 0.854 mmol, 1 equiv) and tert-butyl piperazine-1-carboxylate (318.10 mg, 1.708 mmol, 2 equiv) in dioxane (5 mL) were added Ruphos (79.70 mg, 0.171 mmol, 0.2 equiv) and cesium carbonate (834.68 mg, 2.562 mmol, 3 equiv) in portions at room temperature under argon atmosphere. The resulting mixture was stirred for 1 h at 100° C. under argon atmosphere. The reaction was monitored by TLC. The resulting mixture was concentrated under reduced pressure. The residue was purified by silica gel column chromatography, eluted with petroleum ether/ethyl acetate(1:1) to afford 67a (300 mg, 61.23%) as a white solid. LCMS (ESI, m/z): [M+H]⁺ 574.3

### Step B. 3-(3-methyl-2-oxo-5-(piperazin-1-yl)-2,3-dihydro-1H-benzo[d]imidazol-1-yl)piperidine-2,6-dione (67b)

A mixture of 67a (350 mg, 0.610 mmol, 1 equiv) and trifluoroacetic acid (0.7 mL) in dichloromethane (3.5 mL) was stirred for 1 h at 0° C. The reaction was monitored by TLC. The crude product was used in the next step directly without further purification. LCMS (ESI, m/z): [M+H]⁺ 344.1.

### Step C. tert-butyl N-(3-{4-[1-(2,6-dioxopiperidin-3-yl)-3-methyl-2-oxo-1,3-benzodiazol-5-yl]piperazin-1-yl}propyl)-N-methylcarbamate (67c)

To a stirred mixture of 67b (350 mg, 1.019 mmol, 1 equiv) and tert-butyl N-methyl-N-(3-oxopropyl) carbamate (229.01 mg, 1.223 mmol, 1.2 equiv) in N,N-dimethylformamide (3.5 mL) were added sodium triacetoxyborohydride (648.06 mg, 3.057 mmol, 3 equiv) and acetic acid (6.12 mg, 0.102 mmol, 0.1 equiv) in portions at 50° C. The resulting mixture was stirred for 2 h at room temperature. The reaction was monitored by TLC. The residue was purified by reversed-phase flash chromatography with the following conditions: column, C18 silica gel; mobile phase, acetonitrile in water (10 mmol/L ammonium bicarbonate), 10% to 50% gradient in 10 min; detector, UV 254 nm. This resulted in 67c (200 mg, 38.13%) as a white solid. LCMS (ESI, m/z): [M+H]⁺ 515.2.

### Step D. 3-(3-methyl-5-(4-(3-(methylamino)propyl)piperazin-1-yl)-2-oxo-2,3-dihydro-1H-benzo[d]imidazol-1-yl)piperidine-2,6-dione (CI-67)

A mixture of 67c (60 mg, 0.117 mmol, 1 equiv) and HCl (0.6 mL) in dichloromethane (3 mL) was stirred for 1 h at 0° C. The reaction was monitored by LCMS. The crude product was used in the next step directly without further purification. LCMS (ESI, m/z): [M+H]⁺ 415.2.

### Intermediate 68. 3-(3-methyl-5-(4-(3-(methylamino)propyl)piperazin-1-yl)-2-oxo-2,3-dihydro-1H-benzo[d]imidazol-1-yl)piperidine-2,6-dione (CI-68)

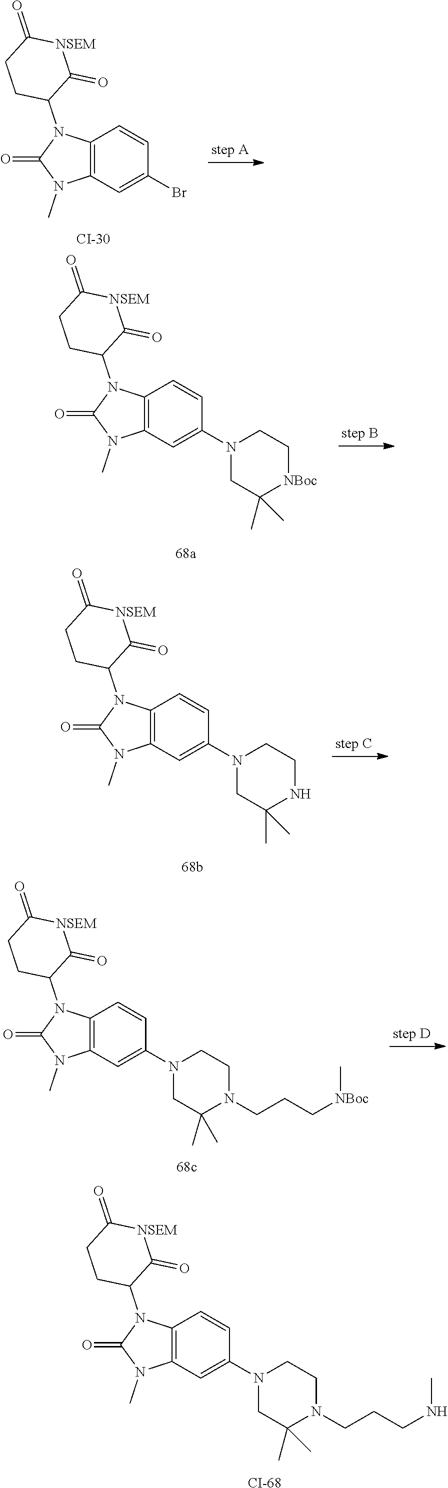

### Step A. tert-butyl 4-[1-(2,6-dioxo-1-{[2-(trimethylsilyl)ethoxy]methyl}piperidin-3-yl)-3-methyl-2-oxo-1,3-benzodiazol-5-yl]-2,2-dimethylpiperazine-1-carboxylate (68a)

To a stirred mixture of CI-30 (400 mg, 0.854 mmol, 1.00 equiv) in dioxane (4 mL) was added RuPhos Palladacycle Gen.3 (71.42 mg, 0.085 mmol, 0.1 equiv) and RuPhos (39.85 mg, 0.085 mmol, 0.1 equiv) and cesium carbonate (556.45 mg, 1.708 mmol, 2 equiv). The resulting mixture was stirred for 1 h at 100° C. Desired product could be detected by LCMS. The resulting mixture was concentrated under reduced pressure. The residue was purified by silica gel column chromatography, eluted with petroleum ether/ethyl acetate (1:1) to afford 68a (360 mg, 61.36%) as a yellow solid. LCMS (ESI, m/z): [M+H]⁺ 602.3.

### Step B. 3-[5-(3,3-dimethylpiperazin-1-yl)-3-methyl-2-oxo-1,3-benzodiazol-1-yl]-1-{[2-(trimethylsilyl)ethoxy]methyl}piperidine-2,6-dione (68b)

To a stirred mixture of 68a (250 mg, 0.415 mmol, 1 equiv) in acetonitrile (12.5 mL) was added HCl (gas) in 1,4-dioxane (2.5 mL) in portions at 0° C. The resulting mixture was stirred for 1 h at 0° C. Desired product could be detected by LCMS. The resulting mixture was concentrated under reduced pressure to afford 68b (250 mg, crude) as a white solid. LCMS (ESI, m/z): [M+H]⁺ 502.3.

### Step C. tert-butyl N-(3-{4-[1-(2,6-dioxo-1-{[2-(trimethylsilyl)ethoxy]methyl}piperidin-3-yl)-3-methyl-2-oxo-1,3-benzodiazol-5-yl]-2,2-dimethylpiperazin-1-yl}propyl)-N-methylcarbamate (68c)

To a stirred mixture of 68b (250 mg, 0.498 mmol, 1 equiv) and tert-butyl N-methyl-N-(3-oxopropyl) carbamate (186.60 mg, 0.996 mmol, 2 equiv) in N,N-dimethylformamide (3 mL) was added acetic acid (8.98 mg, 0.149 mmol, 0.3 equiv). The resulting mixture was stirred for 1 h at 25° C. The resulting mixture was added sodium triacetoxyborohydride (316.83 mg, 1.494 mmol, 3 equiv). The resulting mixture was stirred for 1 h at 25° C. Desired product could be detected by LCMS. The reaction was quenched by the addition of sodium bicarbonate (5 mL). The resulting mixture was extracted with dichloromethane. The combined organic layers were dried over anhydrous sodium sulfate. After filtration, the filtrate was concentrated under reduced pressure. The residue was purified by silica gel column chromatography, eluted with dichloromethane/methanol (20:1) to afford 68c (286 mg, 82.82%) as a white solid. LCMS (ESI, m/z): [M+H]⁺ 673.4.

### Step D. 3-(5-{3,3-dimethyl-4-[3-(methylamino)propyl]piperazin-1-yl}-3-methyl-2-oxo-1,3-benzodiazol-1-yl)-1-{[2-(trimethylsilyl)ethoxy]methyl}piperidine-2,6-dione (CI-68)

To a stirred mixture of 68c (110 mg, 0.163 mmol, 1 equiv) in acetonitrile (5.5 mL) was added HCl (gas) in 1,4-dioxane (1.1 mL) in portions at 0° C. The resulting mixture was stirred for 0.5 h at 0° C. Desired product could be detected by LCMS. The resulting mixture was concentrated under reduced pressure to afford CI-68 (110 mg, crude) as a white solid. LCMS (ESI, m/z): [M+H]⁺ 573.4.

### Intermediate 69. 3-(3-methyl-5-(8-(3-(methylamino)propyl)-3,8-diazabicyclo[3.2.1]octan-3-yl)-2-oxo-2,3-dihydro-1H-benzo[d]imidazol-1-yl)piperidine-2,6-dione (CI-69)

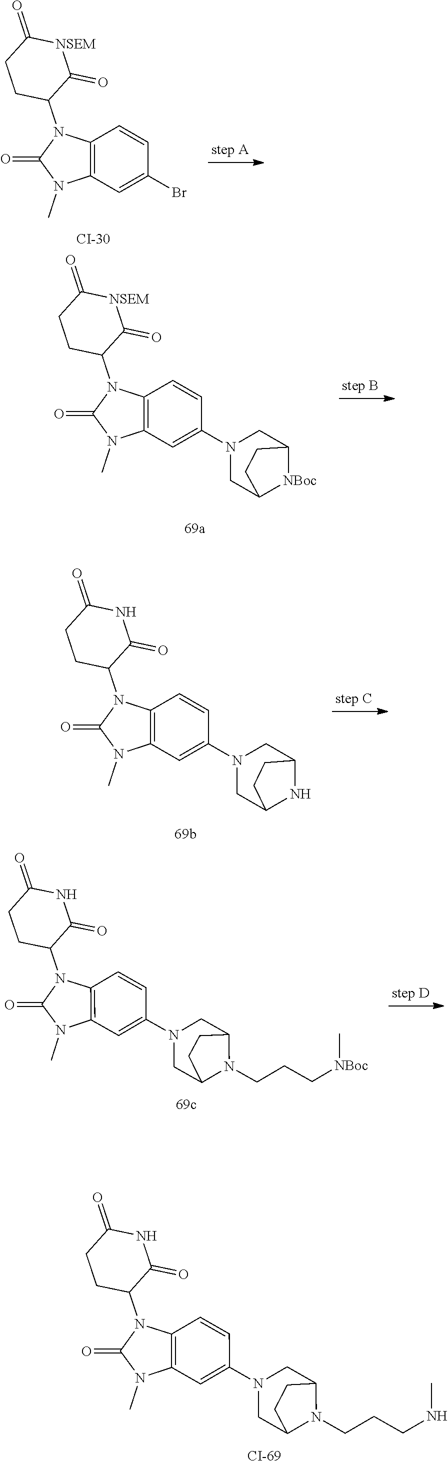

### Step A. tert-butyl 3-(1-(2,6-dioxo-1-((2-(trimethylsilyl)ethoxy)methyl)piperidin-3-yl)-3-methyl-2-oxo-2,3-dihydro-1H-benzo[d]imidazol-5-yl)-3,8-diazabicyclo[3.2.1]octane-8-carboxylate (69a)

To a stirred solution of CI-30 (200 mg, 0.427 mmol, 1 equiv) in dioxane (3 mL) was added tert-butyl 3,8-diazabicyclo[3.2.1]octane-8-carboxylate (181.28 mg, 0.854 mmol, 2 equiv), RuPhos (19.92 mg, 0.043 mmol, 0.1 equiv), RuPhos Palladacycle Gen. 4 (36.29 mg, 0.043 mmol, 0.1 equiv) and cesium carbonate (417.34 mg, 1.281 mmol, 3 equiv). The resulting mixture was stirred for 1 h at 100° C. under nitrogen atmosphere. Desired product could be detected by LCMS. The resulting mixture was diluted with saturated aqueous ammonium chloride (20 mL). The resulting mixture was extracted with ethyl acetate (2×15 mL). The combined organic layers were washed with brine (2×15 mL), dried over anhydrous sodium sulfate. After filtration, the filtrate was concentrated under reduced pressure. The residue was purified by reverse flash chromatography with the following conditions: column, C18 silica gel; mobile phase, acetonitrile in water, 5% to 50% gradient in 30 min; detector, UV 254 nm. Pure fractions were evaporated to dryness to afford 69a (200 mg, 74.19%) as a white solid. LCMS (ESI, m/z): [M+H]⁺ 600.3.

### Step B. tert-butyl (3-(4-(dodec-11-yn-1-yl)-5-oxo-2′,3′,4,5,5′,6′-hexahydro-3H-spiro[benzo[f][1,4]oxazepine-2,4′-pyran]-8-yl)-4-methylphenyl)(tetrahydro-2H-pyran-4-carbonyl)carbamate (69b)

To a stirred solution of 69a (350 mg, 0.584 mmol, 1 equiv) in dichloromethane (1 mL) was added trifluoroacetic acid (3 mL). The resulting mixture was stirred for 1 h at 0° C. under nitrogen atmosphere. Desired product could be detected by LCMS. The mixture was concentrated under reduced pressure to get 200 mg crude product and the residue was used for next step directly. LCMS (ESI, m/z): [M+H]370.1

### Step C. tert-butyl (3-(3-(1-(2,6-dioxopiperidin-3-yl)-3-methyl-2-oxo-2,3-dihydro-1H-benzo[d]imidazol-5-yl)-3,8-diazabicyclo[3.2.1]octan-8-yl)propyl)(methyl)carbamate (69c)

To a stirred solution of 69b (200 mg, 0.541 mmol, 1 equiv) in N,N-dimethylformamide (3 mL) was added tert-butyl N-methyl-N-(3-oxopropyl)carbamate (121.64 mg, 0.649 mmol, 1.2 equiv) and sodium triacetoxyborohydride (344.22 mg, 1.623 mmol, 3 equiv). The resulting mixture was stirred for 3 h at 50° C. under nitrogen atmosphere. Desired product could be detected by LCMS. The residue was purified by reverse flash chromatography with the following conditions: column, C18 silica gel; mobile phase, acetonitrile in water, 5% to 50% gradient in 30 min; detector, UV 254 nm. Pure fractions were evaporated to dryness to afford 69c (90 mg, 27.67%) as a white solid. LCMS (ESI, m/z): [M+H]⁺ 441.2.

### Step D. 3-(3-methyl-5-(8-(3-(methylamino)propyl)-3,8-diazabicyclo[3.2.1]octan-3-yl)-2-oxo-2,3-dihydro-1H-benzo[d]imidazol-1-yl)piperidine-2,6-dione (CI-69)

To a stirred mixture of 69c (90 mg, 0.163 mmol, 1 equiv) in acetonitrile (5.5 mL) was added HCl (gas) in 1,4-dioxane (1.1 mL) in portions at 0° C. The resulting mixture was stirred for 0.5 h at 0° C. Desired product could be detected by LCMS. The resulting mixture was concentrated under reduced pressure to afford CI-69 (90 mg, crude) as a white solid. LCMS (ESI, m/z): [M+H]⁺ 573.4.

### Intermediate 70. 3-(3-methyl-5-(5-(3-(methylamino)propyl)-2,5-diazabicyclo[2.2.11 heptan-2-yl)-2-oxo-2,3-dihydro-1H-benzo[d]imidazol-1-yl)piperidine-2,6-dione (CI-70)

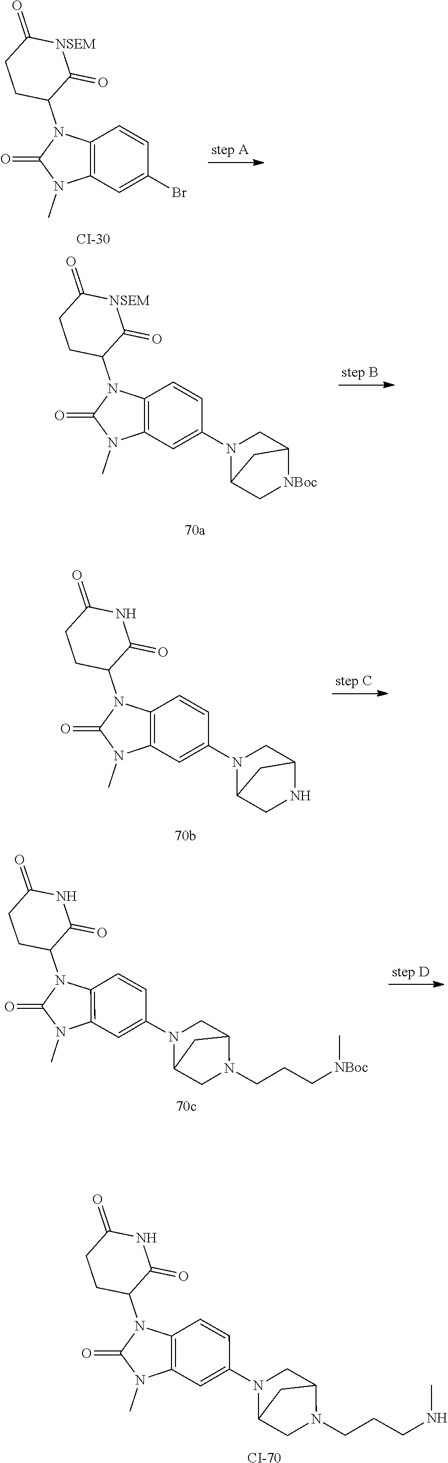

### Step A. (3S)-3-[3-methyl-2-oxo-4-(piperazin-1-yl)-1,3-benzodiazol-1-yl]piperidine-2,6-dione (70a)

To a solution of CI-30 (500 mg, 1.067 mmol, 1 equiv) in 1,4-dioxane (5 mL) was added tert-butyl 3,6-diazabicyclo[3.1.1]heptane-6-carboxylate (211.63 mg, 1.067 mmol, 1 equiv), RuPhos palladacycle Gen.3 (89.28 mg, 0.107 mmol, 0.1 equiv), RuPhos (49.81 mg, 0.107 mmol, 0.1 equiv), cesium carbonate (1043.35 mg, 3.201 mmol, 3 equiv) at 100° C. The mixture was stirred for 2 h. Desired product could be detected by LCMS. The resulting mixture was concentrated under reduced pressure. The residue was purified by silica gel column chromatography, eluted with dichloromethane/methanol (30/1) to afford 70a (320 mg, 51.18%) as a yellow solid. LCMS (ESI, m/z): [M+H]⁺=586.3.

### Step B. 3-(5-{2,5-diazabicyclo[2.2.1]heptan-2-yl}-3-methyl-2-oxo-1,3-benzodiazol-1-yl)piperidine-2,6-dione (70b)

To a solution 70a (320 mg, 0.546 mmol, 1 equiv) in dichloromethane (3 mL) was added HCl (gas) in 1,4-dioxane (0.50 mL, 16.38 mmol, 30 equiv) at 0° C. The mixture was stirred for 30 min. Desired product could be detected by LCMS. The resulting mixture was concentrated under reduced pressure. The residue was purified by reversed-phase flash chromatography with the following conditions: column, C18 silica gel; mobile phase, water in acetonitrile, 10% to 50% gradient in 20 min; detector, UV 254 nm. Pure fractions were evaporated to dryness to afford 70b (150 mg, 77.26%). LCMS (ESI, m/z): [M+H]⁺=486.3.

### Step C. tert-butyl N-(3-{5-[1-(2,6-dioxopiperidin-3-yl)-3-methyl-2-oxo-1,3-benzodiazol-5-yl]-2,5-diazabicyclo[2.2.1]heptan-2-yl}propyl)-N-methylcarbamate (70c)

To a solution of 70b (150 mg, 0.422 mmol, 1 equiv) in N,N-dimethylformamide (2 mg) was added tert-butyl N-methyl-N-(3-oxopropyl)carbamate (79.03 mg, 0.422 mmol, 1 equiv), acetic acid (2.53 mg, 0.042 mmol, 0.10 equiv) at room temperature. The mixture was stirred for 30 min. Adding sodium triacetoxyborohydride (268.36 mg, 1.266 mmol, 3.00 equiv) and tetraethyl titanate (962.77 mg, 4.220 mmol, 10 equiv) at mixture. The mixture was stirred for 1 h. Desired product could be detected by LCMS. The resulting mixture was extracted with ethyl acetate (3×30 mL). After filtration, the filtrate was concentrated under reduced pressure. The residue was purified by silica gel column chromatography, eluted with dichloromethane/methanol (30/1) to afford 70c (120 mg, 53.99%) as a white solid. LCMS (ESI, m/z): [M+H]⁺=527.3.

### Step D. 3-(3-methyl-5-(8-(3-(methylamino)propyl)-3,8-diazabicyclo[3.2.1]octan-3-yl)-2-oxo-2,3-dihydro-1H-benzo[d]imidazol-1-yl)piperidine-2,6-dione (CI-70)

To a stirred mixture of 70c (80 mg, 0.163 mmol, 1 equiv) in acetonitrile (5.5 mL) was added HCl (gas) in 1,4-dioxane (1.1 mL) in portions at 0° C. The resulting mixture was 15 stirred for 0.5 h at 0° C. Desired product could be detected by LCMS. The resulting mixture was concentrated under reduced pressure to afford CI-70 (70 mg, crude) as a white solid. LCMS (ESI, m/z): [M+H]⁺ 573.4.

### Intermediate 71. 3-(3-methyl-5-{6-[3-(methylamino)propyl]-3,6-diazabicyclo[3.1.1]heptan-3-yl]-2-oxo-1,3-benzodiazol-1-yl)piperidine-2,6-dione (CI-71)

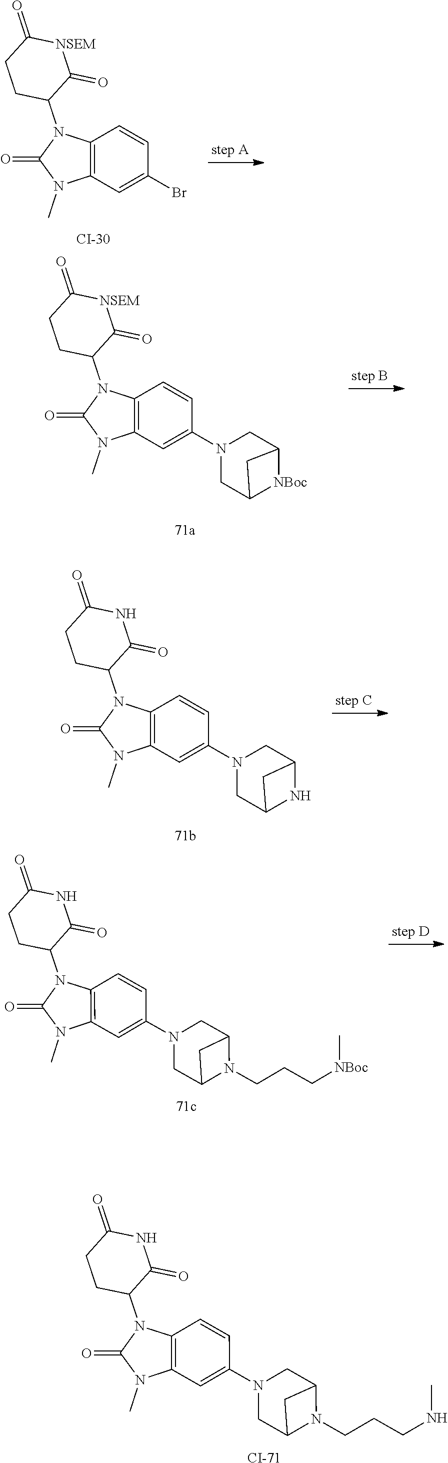

### Step A. tert-butyl 3-[1-(2,6-dioxo-1-{[2-(trimethylsilyl)ethoxy]methyl}piperidin-3-yl)-3-methyl-2-oxo-1,3-benzodiazol-5-yl]-3,6-diazabicyclo[3.1.1]heptane-6-carboxylate (71a)

To a solution of CI-30 (500 mg, 1.067 mmol, 1 equiv) in 1,4-dioxane (5 mL) was added tert-butyl 3,6-diazabicyclo[3.1.1]heptane-6-carboxylate (211.63 mg, 1.067 mmol, 1 equiv), RuPhos palladacycle Gen.3 (89.28 mg, 0.107 mmol, 0.1 equiv), RuPhos (49.81 mg, 0.107 mmol, 0.1 equiv), cesium carbonate (1043.35 mg, 3.201 mmol, 3 equiv) at room temperature. The mixture was stirred for 2 h. Desired product could be detected by LCMS. The resulting mixture was concentrated under reduced pressure. The residue was purified by silica gel column chromatography, eluted with dichloromethane/methanol (30/1) to afford 71a (280 mg, 44.78%) as a yellow solid. LCMS (ESI, m/z): [M+H]⁺ 586.3.

### Step B. 3-(5-{3,6-diazabicyclo[3.1.1]heptan-3-yl}-3-methyl-2-oxo-1,3-benzodiazol-1-yl)piperidine-2,6-dione (71b)

To a solution of 71a (280 mg, 0.478 mmol, 1 equiv) in dichloromethane (3 mL) was 15 added HCl (gas) in 1,4-dioxane (0.44 mL, 14.340 mmol, 30 equiv) at 0° C. The mixture was stirred for 30 min. Desired product could be detected by LCMS. The resulting mixture was concentrated under reduced pressure. The residue was purified by reversed-phase flash chromatography with the following conditions: column, C18 silica gel; mobile phase, water in acetonitrile, 10% to 50% gradient in 20 min; detector, UV 254 nm to afford 71b (120 mg, 70.64%). LCMS (ESI, m/z): [M+H]⁺ 486.3.

### Step C. tert-butyl N-(3-{3-[1-(2,6-dioxopiperidin-3-yl)-3-methyl-2-oxo-1,3-benzodiazol-5-yl]-3,6-diazabicyclo[3.1.1]heptan-6-yl}propyl)-N-methylcarbamate (71c)

To a solution of 71b (120 mg, 0.338 mmol, 1 equiv) in N,N-dimethylformamide (2 mg) was added tert-butyl N-methyl-N-(3-oxopropyl)carbamate (63.22 mg, 0.338 mmol, 1 equiv), acetic acid (0.1 mL, 1.745 mmol, 5.17 equiv) at room temperature. The mixture was stirred for 30 min. Adding sodium triacetoxyborohydride (214.68 mg, 1.014 mmol, 3 equiv) and tetraethyl titanate (770.22 mg, 3.380 mmol, 10 equiv) at mixture. The mixture was stirred for 1 h. Desired product could be detected by LCMS. The resulting mixture was extracted with ethyl acetate (3×30 mL). After filtration, the filtrate was concentrated under reduced pressure. The residue was purified by silica gel column chromatography, eluted with dichloromethane/methanol (30/1) to afford 71c (100 mg, 56.24%) as a white solid. LCMS (ESI, m/z): [M+H]⁺ 527.3.

### Step D. 3-(3-methyl-5-{6-[3-(methylamino)propyl]-3,6-diazabicyclo[3.1.1]heptan-3-yl}-2-oxo-1,3-benzodiazol-1-yl)piperidine-2,6-dione (CI-71)

To a solution of 71c (100 mg, 0.190 mmol, 1 equiv) in dichloromethane (1 mL) was added HCl (gas) in 1,4-dioxane (0.06 mL, 1.900 mmol, 10 equiv) at 0° C. The mixture was stirred for 30 min. Desired product could be detected by LCMS. The resulting mixture was concentrated under reduced pressure. The residue was purified by reversed-phase flash chromatography with the following conditions: column, C18 silica gel; mobile phase, water in acetonitrile, 10% to 50% gradient in 20 min; detector, UV 254 nm to afford CI-71 (70 mg, 86.43%) as white solid. LCMS (ESI, m/z): [M+H]⁺=427.2.

### Intermediate 72. 3-{3-methyl-2-oxo-5-[1-(pyrrolidin-3-ylmethyl)piperidin-4-yl}-1,3-benzodiazol-1-yl}piperidine-2,6-dione (CI-72)

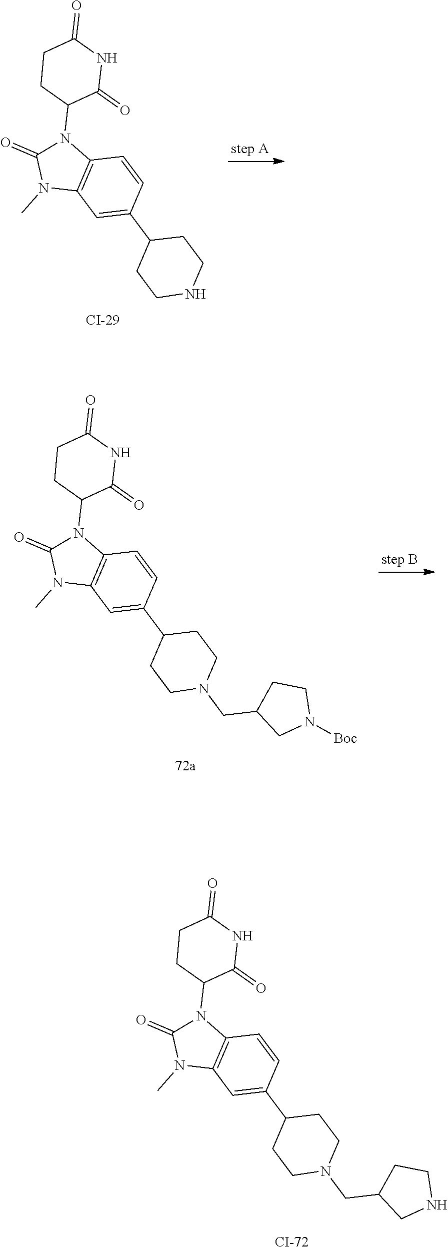 [SMILES: Cn1c(=O)n(C2CCC(=O)NC2=O)c2ccc(C3CCNCC3)cc21 ; "CI-29" ; Cn1c(=O)n(C2CCC(=O)NC2=O)c2ccc(C3CCN(CC4CCN(C(=O)OC(C)(C)C)C4)CC3)cc21 ; "72a" ; Cn1c(=O)n(C2CCC(=O)NC2=O)c2ccc(C3CCN(CC4CCNC4)CC3)cc21 ; "CI-72"]

### Step A. tert-butyl 3-({4-[1-(2,6-dioxopiperidin-3-yl)-3-methyl-2-oxo-1,3-benzodiazol-5-yl]piperidin-1-yl}methyl)pyrrolidine-1-carboxylate (72a)

A solution of CI-29 (500 mg, 1.460 mmol, 1 equiv) and tert-butyl 3-formylpyrrolidine-1-carboxylate (320.06 mg, 1.606 mmol, 1.1 equiv) in N,N-dimethylformamide (10 mL) were treated with acetic acid (26.31 mg, 0.438 mmol, 0.3 equiv) for 1 h at 50° C. under nitrogen atmosphere followed by the addition of sodium triacetoxyborohydride (928.48 mg, 4.380 mmol, 3 equiv) in portions at room temperature. The resulting mixture was stirred for 1 h at room temperature under nitrogen atmosphere. Desired product could be detected by LCMS. The residue was purified by reversed-phase flash chromatography with the following conditions: column, C18 silica gel; mobile phase, acetonitrile in water (10 mmol/L ammonium bicarbonate), 10% to 70% gradient in 10 min; detector, UV 254 nm. This resulted in 72a (600 mg, 78.17%) as a white solid. LCMS (ESI, m/z): [M+H]⁺ 526.6.

### Step B. 3-{3-methyl-2-oxo-5-[1-(pyrrolidin-3-ylmethyl)piperidin-4-yl]-1,3-benzodiazol-1-yl}piperidine-2,6-dione (CI-72)

A solution of 72a (200 mg, 0.380 mmol, 1 equiv) and HCl (gas) in 1,4-dioxane (2 mL, 65.826 mmol, 173.01 equiv) in acetonitrile (2 mL) was stirred for 1 h at 0° C. under nitrogen atmosphere. Desired product could be detected by LCMS. The resulting mixture was concentrated under reduced pressure. The crude product was used in the next step directly without further purification. This resulted in CI-72 (150 mg, 92.65%) as a white solid. LCMS (ESI, m/z): [M+H]⁺ 426.5.

### Intermediate 73. 3-{3-methyl-2-oxo-5-[1-(piperidin-3-ylmethyl)piperidin-4-yl}-1,3-benzodiazol-1-yl}piperidine-2,6-dione (CI-73)

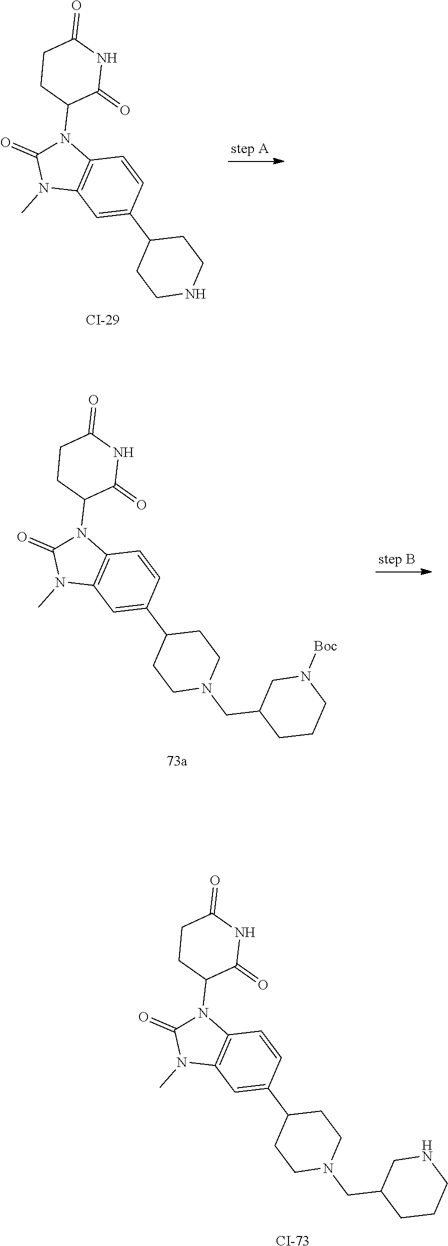 [SMILES: Cn1c(=O)n(C2CCC(=O)NC2=O)c2ccc(C3CCNCC3)cc21 ; "CI-29" ; Cn1c(=O)n(C2CCC(=O)NC2=O)c2ccc(C3CCN(CC4CCCN(C(=O)OC(C)(C)C)C4)CC3)cc21 ; "73a" ; Cn1c(=O)n(C2CCC(=O)NC2=O)c2ccc(C3CCN(CC4CCCNC4)CC3)cc21 ; "CI-73"]

### Step A. tert-butyl 3-({4-[1-(2,6-dioxopiperidin-3-yl)-3-methyl-2-oxo-1,3-benzodiazol-5-yl]piperidin-1-yl}methyl)piperidine-1-carboxylate (73a)

A solution of CI-29 (200 mg, 0.584 mmol, 1 equiv), tert-butyl 3-formylpiperidine-1-carboxylate (249.16 mg, 1.168 mmol, 2 equiv), sodium triacetoxyborohydride (371.39 mg, 1.752 mmol, 3 equiv) and acetic acid (350.77 mg, 5.840 mmol, 10 equiv) in N,N-dimethylformamide (3 mL) was stirred for 1 h at room temperature under air atmosphere. Desired product could be detected by LCMS. The reaction was quenched with Water at room temperature. The aqueous layer was extracted with ethyl acetate (3×10 mL). The resulting mixture was concentrated under reduced pressure. The residue was purified by silica gel column chromatography, eluted with petroleum ether/ethyl acetate(1:1) to afford 73a (240 mg, 76.13%) as a white solid. LCMS (ESI, m/z): [M+H]⁺ 539.3.

### Step B. 3-{3-methyl-2-oxo-5-[1-(piperidin-3-ylmethyl)piperidin-4-yl]-1,3-benzodiazol-1-yl}piperidine-2,6-dione (CI-73)

A solution of 73a (200 mg, 0.371 mmol, 1 equiv) and HCl (gas) in 1,4-dioxane (1 mL, 4.000 mmol, 10.79 equiv) in dichloromethane (3 mL, 47.192 mmol, 127.34 equiv) was stirred for 1 h at room temperature under air atmosphere. Desired product could be detected by LCMS. The resulting mixture was concentrated under reduced pressure. This resulted in CI-73 (150 mg, 92.08%) as a white solid. LCMS (ESI, m/z): [M+H]⁺ 439.3.

### Intermediate 74. 5-[4-(7-[1-(1-ethoxyethyl)pyrazol-4-yl]-8-isopropoxy-11,2,41triazolo[1,5-c]pyrimidin-2-yl}amino)-3-fluorobenzenesulfonyl]furan-2-carbaldehyde (CI-74)

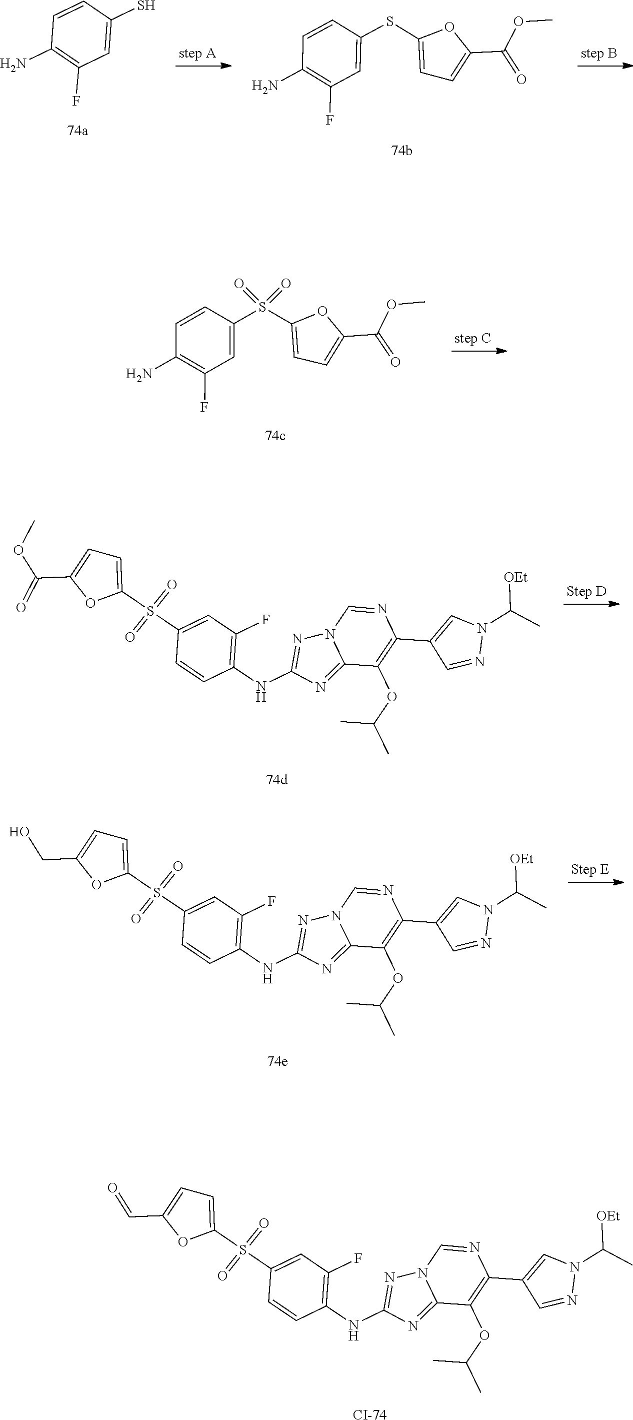

### Step A. methyl 5-[(4-amino-3-fluorophenyl) sulfanyl]furan-2-carboxylate (74b)

To a stirred mixture of 74a (2 g, 13.968 mmol, 1 equiv), potassium phosphate tribasic (5.93 g, 27.936 mmol, 2 equiv) and methyl 5-bromofuran-2-carboxylate (3.15 g, 15.365 mmol, 1.1 equiv) in dioxane (4 mL) were added cuprous iodide (0.27 g, 1.397 mmol, 0.1 equiv) and 1,10-phenanthroline (0.50 g, 2.794 mmol, 0.2 equiv) in portions at room temperature under nitrogen atmosphere. The resulting mixture was stirred for 40 min at 100° C. under nitrogen atmosphere. Desired product could be detected by LCMS. The residue was purified by silica gel column chromatography, eluted with petroleum ether/ethyl acetate(5:1) to afford 74b (1 g, 26.79%) as a yellow solid. LCMS (ESI, m/z): [M+H]⁺ 268.0.

### Step B. methyl 5-(4-amino-3-fluorobenzenesulfonyl) furan-2-carboxylate (74c)

A solution of 74b (2 g, 7.483 mmol, 1 equiv) and m-chloroperoxybenzoic acid (3.87 g, 22.449 mmol, 3 equiv) in dichloromethane (10 mL) was stirred for 1 h at room temperature under nitrogen atmosphere. Desired product could be detected by LCMS. The residue was purified by silica gel column chromatography, eluted with petroleum ether/ethyl acetate(1:1) to afford 74c (1 g, 44.65%) as a yellow solid. LCMS (ESI, m/z): [M+H]300.0.

### Step C. methyl 5-[4-({7-[1-(1-ethoxyethyl)pyrazol-4-yl]-8-isopropoxy-[1,2,4]triazolo[1,5-c]pyrimidin-2-yl}amino)-3-fluorobenzenesulfonyl]furan-2-carboxylate (74d)

To a stirred solution of 74c (1 g, 3.341 mmol, 1 equiv), CI-1 (1.32 g, 3.341 mmol, 1 equiv) and RuPhos (311.86 mg, 0.668 mmol, 0.2 equiv) in dioxane (10 mL) were added Pd₂(dba)₃ (611.98 mg, 0.668 mmol, 0.2 equiv) and cesium carbonate (3.27 g, 10.023 mmol, 3 equiv) in portions at room temperature under nitrogen atmosphere. The resulting mixture was stirred for 1 h at 80° C. under nitrogen atmosphere. Desired product could be detected by LCMS. The residue was purified by silica gel column chromatography, eluted with petroleum ether/ethyl acetate(5:1) to afford 74d (600 mg, 29.26%) as a yellow solid. LCMS (ESI, m/z): [M+H]⁺ 614.2.

### Step D. {5-[4-({7-[1-(1-ethoxyethyl)pyrazol-4-yl]-8-isopropoxy-[1,2,4]triazolo[1,5-c]pyrimidin-2-yl}amino)-3-fluorobenzenesulfonyl]furan-2-yl}methanol (74e)

To a stirred solution of 74d (600 mg, 0.978 mmol, 1 equiv) in tetrahydrofuran (5 mL) was added DIBAl-H (1390.65 mg, 9.780 mmol, 10 equiv) in portions at 0° C. under nitrogen atmosphere. The resulting mixture was stirred for 1 h at 0° C. under nitrogen atmosphere. Desired product could be detected by LCMS. The reaction was quenched with anhydrous sodium sulfate. 10water at 0° C. The resulting mixture was filtered, the filter cake was washed with dichloromethane (3×50 mL). The filtrate was concentrated under reduced pressure. The residue was purified by silica gel column chromatography, eluted with petroleum ether/ethyl acetate(1:1) to afford 74e (300 mg, 52.39%) as a yellow solid. LCMS (ESI, m/z): [M+H]⁺ 586.2.

### Step E. 5-[4-({7-[1-(1-ethoxyethyl)pyrazol-4-yl]-8-isopropoxy-[1,2,4]triazolo[1,5-c]pyrimidin-2-yl}amino)-3-fluorobenzenesulfonyl]furan-2-carbaldehyde (CI-74)

A solution of 74e (270 mg, 0.461 mmol, 1 equiv) and manganese dioxide (801.65 mg, 9.220 mmol, 20 equiv) in dichloromethane (5 mL) was stirred for 1 day at room temperature under nitrogen atmosphere. Desired product could be detected by LCMS. The resulting mixture was filtered, the filter cake was washed with dichloromethane (3×50 mL). The filtrate was concentrated under reduced pressure. The residue was purified by silica gel column chromatography, eluted with petroleum ether/ethyl acetate(1:1) to afford CI-74 (100 mg, 37.16%) as a yellow solid. LCMS (ESI, m/z): [M+H]⁺ 584.2.

### Intermediate 75. 1-(1-(2,6-dioxo-1-((2-(trimethylsilyl)ethoxy)methyl)piperidin-3-yl)-3-methyl-2-oxo-2,3-dihydro-1H-benzo[d]imidazol-4-yl)piperidine-4-carbaldehyde(CI-75)

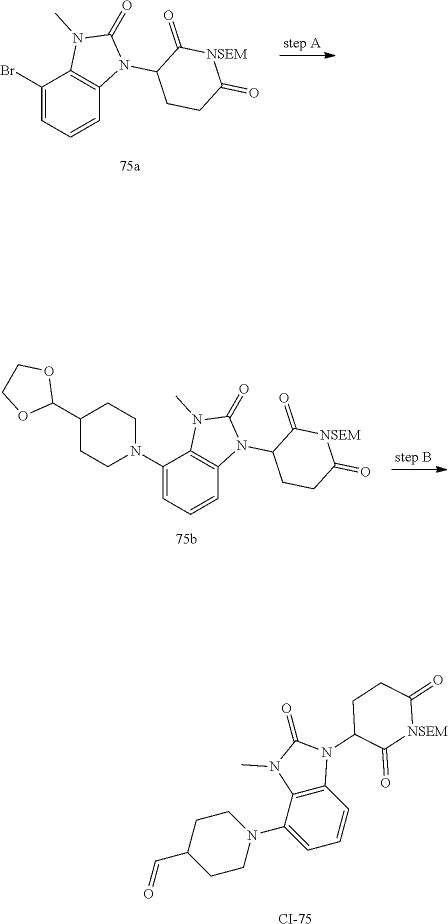 [SMILES: Cn1c(=O)n(C2CCC(=O)N(COCC[Si](C)(C)C)C2=O)c2cccc(Br)c21 ; Cn1c(=O)n(C2CCC(=O)N(COCC[Si](C)(C)C)C2=O)c2cccc(N3CCC(C4OCCO4)CC3)c21 ; Cn1c(=O)n(C2CCC(=O)N(COCC[Si](C)(C)C)C2=O)c2cccc(N3CCC(C=O)CC3)c21 ; "CI-75"]

### Step A. 3-(4-(4-(1,3-dioxolan-2-yl)piperidin-1-yl)-3-methyl-2-oxo-2,3-dihydro-1H-benzo[d]imidazol-1-yl)-1-((2-(trimethylsilyl)ethoxy)methyl)piperidine-2,6-dione (75b)

To a stirred mixture of 75a (400 mg, 0.854 mmol, 1 equiv) and 4-(1,3-dioxolan-2-yl)piperidine (134.25 mg, 0.854 mmol, 1 equiv) and Pd-PEPPSI-IPentCl 2-methylpyridine (o-picoline) (71.83 mg, 0.085 mmol, 0.1 equiv) in dioxane (10 mL) were added potassium carbonate (354.05 mg, 2.562 mmol, 3 equiv) at 25° C. under N₂. The resulting mixture was stirred for 12 h at 110° C. Desired product could be detected by LCMS. The mixture was concentrated under vacuum. The resulting mixture was diluted with water (50 mL) and extracted with ethyl acetate (3×100 mL). The combined organic layers were dried over anhydrous sodium sulfate and concentrated under reduced pressure. The residue was purified by reversed-phase Combi-Flash, eluted with acetonitrile:water (0.5% ammonium bicarbonate)=5%-60%.This resulted in 75b (210 mg, 33.86%) as a yellow solid. LCMS (ESI, m/z): [M+H]⁺ 545.3.

### Step B. 1-(1-(2,6-dioxo-1-((2-(trimethylsilyl)ethoxy)methyl)piperidin-3-yl)-3-methyl-2-oxo-2,3-dihydro-1H-benzo[d]imidazol-4-yl)piperidine-4-carbaldehyde (CI-75)

To a stirred solution of 75b (100 mg, 0.184 mmol, 1 equiv) in acetone (5 mL) and water (1 mL) was added p-toluenesulfonic acid (126.45 mg, 0.736 mmol, 4 equiv) at 25° C. under N₂. The resulting mixture was stirred for 1 h at 60° C. Desired product could be detected by LCMS. The mixture was concentrated under vacuum. The resulting mixture was diluted with water (50 mL) and extracted with ethyl acetate (3×100 mL). The combined organic layers were dried over anhydrous sodium sulfate and concentrated under reduced pressure. The residue was purified by reversed-phase Combi-Flash, eluted with acetonitrile:water (0.5% ammonium bicarbonate)=5%-60%.This resulted in CI-75 (60 mg, 48.96%) as a yellow solid. LCMS (ESI, m/z): [M+H]⁺ 501.2.

### Intermediate 76. N-(4-{2,6-diazabicyclo[3.2.0]heptane-6-sulfonyl]-2-fluorophenyl)-8-isopropoxy-7-(1H-pyrazol-4-yl)-[1,2,4]triazolo[1,5-c]pyrimidin-2-amine (CI-76)

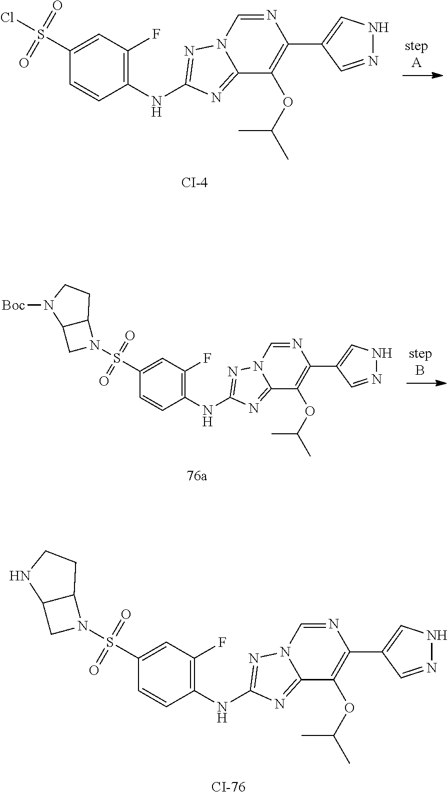 [SMILES: CC(C)Oc1c(-c2cn[nH]c2)ncn2nc(Nc3ccc(S(=O)(=O)Cl)cc3F)nc12 ; "CI-4" ; CC(C)Oc1c(-c2cn[nH]c2)ncn2nc(Nc3ccc(S(=O)(=O)N4CC5C4CCN5C(=O)OC(C)(C)C)cc3F)nc12 ; "76a" ; CC(C)Oc1c(-c2cn[nH]c2)ncn2nc(Nc3ccc(S(=O)(=O)N4CC5NCCC54)cc3F)nc12 ; "CI-76"]

### Step A. tert-butyl 6-(3-fluoro-4-{[8-isopropoxy-7-(1H-pyrazol-4-yl)-[1,2,4]triazolo[1,5-c]pyrimidin-2-yl]amino}benzenesulfonyl)-2,6-diazabicyclo[3.2.0]heptane-2-carboxylate (76a)

To a stirred solution of CI-4 (600 mg, 1.328 mmol, 1 equiv) and tert-butyl 2,6-diazabicyclo[3.2.0]heptane-2-carboxylate (263.27 mg, 1.328 mmol, 1 equiv) in acetonitrile (5 mL) was added trimethylamine (537.47 mg, 5.312 mmol, 4 equiv) in portions at 0° C. under nitrogen atmosphere. The resulting mixture was stirred for 1 h at 0° C. under nitrogen atmosphere. Desired product could be detected by LCMS. The resulting mixture was concentrated under reduced pressure. The residue was purified by reversed-phase flash chromatography with the following conditions: column, C18 silica gel; mobile phase, acetonitrile in water (0.1% formic acid), 10% to 60% gradient in 10 min; detector, UV 254 nm. This resulted in 76a (500 mg, 61.36%) as a white solid. LCMS (ESI, m/z): [M+H]⁺ 613.6.

### Step B. N-(4-{2,6-diazabicyclo[3.2.0]heptane-6-sulfonyl}-2-fluorophenyl)-8-isopropoxy-7-(1H-pyrazol-4-yl)-[1,2,4]triazolo[1,5-c]pyrimidin-2-amine (CI-76)

A solution of 76a (200 mg, 0.326 mmol, 1 equiv) and HCl (gas) in 1,4-dioxane (1 mL, 32.913 mmol, 100.99 equiv) in acetonitrile (1 mL) was stirred for 1 h at 0° C. under nitrogen atmosphere. Desired product could be detected by LCMS. The resulting mixture was concentrated under reduced pressure. The crude product mixture was used in the next step directly without further purification. This resulted in CI-76 (100 mg, 59.75%) as a white solid. LCMS (ESI, m/z): [M+H]⁺ 514.5.

### Intermediate 77. N-{4-[7-(azetidin-3-ylmethyl)-2,7-diazaspiro[4.4]nonan-2-yl]sulfonyl]-2-fluorophenyl]-8-isopropoxy-7-(1H-pyrazol-4-yl)-[1,2,4]triazolo[1,5-c]pyrimidin-2-amine (CI-77)

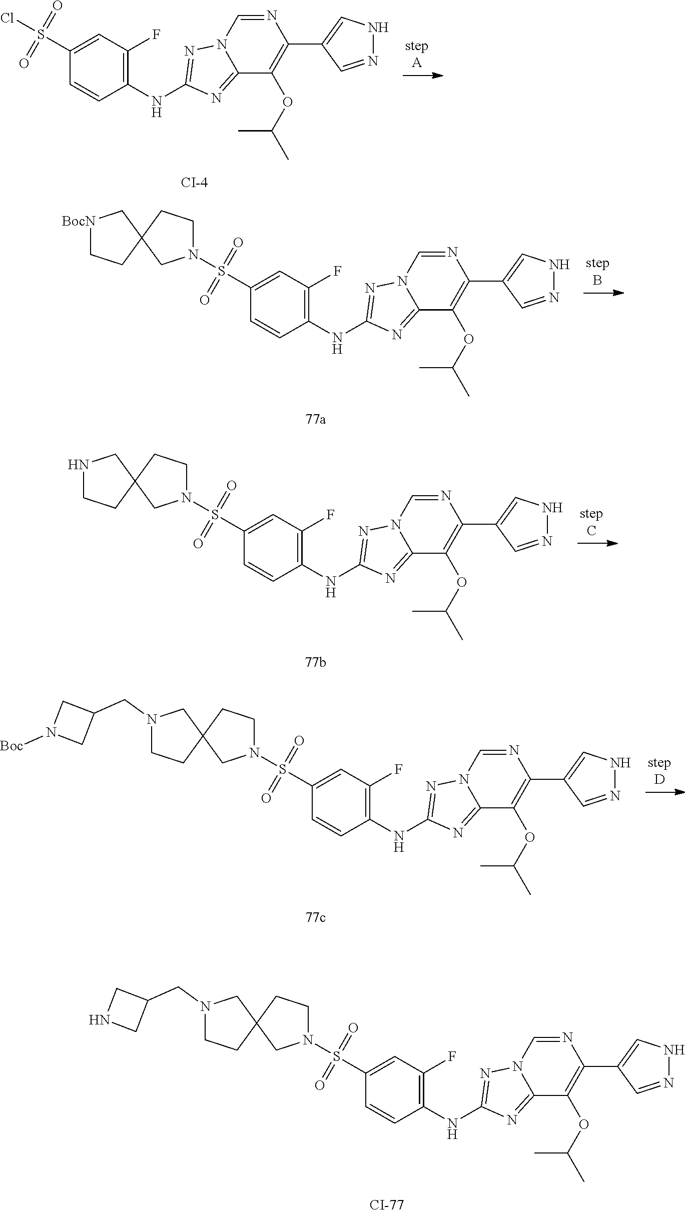

### Step A. tert-butyl 7-(3-fluoro-4-{[8-isopropoxy-7-(1H-pyrazol-4-yl)-[1,2,4]triazolo[1,5-c]pyrimidin-2-yl]amino}benzenesulfonyl)-2,7-diazaspiro[4.4]nonane-2-carboxylate (77a)

A solution of CI-4 (400 mg, 0.885 mmol, 1 equiv), tert-butyl 2,7-diazaspiro[4.4]nonane-2-carboxylate (400.69 mg, 1.770 mmol, 2 equiv) and trimethylamine (268.74 mg, 2.655 mmol, 3 equiv) in dichloromethane (5 mL) was stirred for 30 min at 0° C. under air atmosphere. Desired product could be detected by LCMS. The resulting mixture was concentrated under reduced pressure. The residue was purified by Prep-TLC (petroleum ether/ethyl acetate 5:1) to afford 77a (400 mg, 70.41%) as a white solid. LCMS (ESI, m/z): [M+H]⁺ 642.2

### Step B. N-(4-{2,7-diazaspiro[4.4]nonane-2-sulfonyl}-2-fluorophenyl)-8-isopropoxy-7-(1H-pyrazol-4-yl)-[1,2,4]triazolo[1,5-c]pyrimidin-2-amine (77b)

A solution of 77a (350 mg, 0.545 mmol, 1 equiv) and trifluoroacetic acid (0.5 mL, 6.732 mmol, 12.34 equiv) in dichloromethane (4 mL) was stirred for 1 h at room temperature under nitrogen atmosphere. Desired product could be detected by LCMS. The resulting mixture was concentrated under reduced pressure. The residue was purified by silica gel column chromatography, eluted with petroleum ether/ethyl acetate(1:1) to afford 77b (250 mg, 84.63%) as a white solid. LCMS (ESI, m/z): [M+H]⁺ 542.2

### Step C. tert-butyl 3-{1[7-(3-fluoro-4-{1[8-isopropoxy-7-(1H-pyrazol-4-yl)-[1,2,4]triazolo[1,5-c]pyrimidin-2-yl]amino}benzenesulfonyl)-2,7-diazaspiro[4.4]nonan-2-yl]methyl}azetidine-1-carboxylate (77c)

A solution of 77b (300 mg, 0.554 mmol, 1 equiv), tert-butyl 3-formylazetidine-1-carboxylate (205.19 mg, 1.108 mmol, 2 equiv), sodium triacetoxyborohydride (352.18 mg, 1.662 mmol, 3 equiv) and acetic acid (332.63 mg, 5.540 mmol, 10 equiv) in dichloromethane (4 mL) was stirred for 1 h at 0° C. under air atmosphere. Desired product could be detected by LCMS. The reaction was quenched with Water at room temperature. The aqueous layer was extracted with ethyl acetate (3×10 mL). The resulting mixture was concentrated under reduced pressure. The residue was purified by silica gel column chromatography, eluted with dichloromethane/methanol (10:1) to afford 77c (280 mg, 71.11%) as a white solid. LCMS (ESI, m/z): [M+H]⁺ 711.3.

### Step D. N-{4-[7-(azetidin-3-ylmethyl)-2,7-diazaspiro[4.4]nonan-2-ylsulfonyl]-2-fluorophenyl}-8-isopropoxy-7-(1H-pyrazol-4-yl)-[1,2,4]triazolo[1,5-c]pyrimidin-2-amine (CI-77)

A solution of 77c (250 mg, 0.352 mmol, 1 equiv) and HCl (gas) in 1,4-dioxane (1 mL, 4.000 mmol, 11.37 equiv) in dichloromethane (3 mL) was stirred for 1 h at room temperature under nitrogen atmosphere. Desired product could be detected by LCMS. The resulting mixture was concentrated under reduced pressure. The residue was purified by silica gel column chromatography, eluted with dichloromethane/methanol (10:1) to afford CI-77 (200 mg, 93.12%) as a white solid. LCMS (ESI, m/z): [M+H]⁺ 611.2.

### Intermediate 78. N-(4-((2,6-diazaspiro[3.51nonan-2-yl)sulfonyl)-2-fluorophenyl)-8-isopropoxy-7-(1H-pyrazol-4-yl)-[1,2,4]triazolo[1,5-c]pyrimidin-2-amine (CI-78)

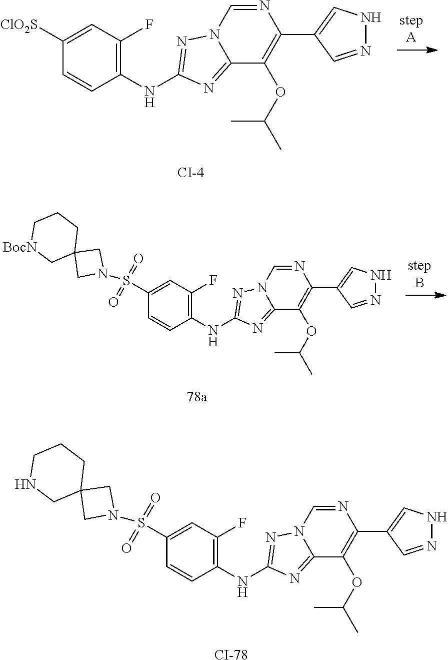 [SMILES: CC(C)Oc1c(-c2cn[nH]c2)ncn2nc(Nc3ccc(S(=O)(=O)Cl)cc3F)nc12 ; "CI-4" ; CC(C)Oc1c(-c2cn[nH]c2)ncn2nc(Nc3ccc(S(=O)(=O)N4CC5(CCCN(C(=O)OC(C)(C)C)C5)C4)cc3F)nc12 ; "78a" ; CC(C)Oc1c(-c2cn[nH]c2)ncn2nc(Nc3ccc(S(=O)(=O)N4CC5(CCCNC5)C4)cc3F)nc12 ; "CI-78"]

### Step A. tert-butyl 2-((3-fluoro-4-((8-isopropoxy-7-(1H-pyrazol-4-yl)-[1,2,4]triazolo[1,5-c]pyrimidin-2-yl)amino)phenyl)sulfonyl)-2,6-diazaspiro[3.5]nonane-6-carboxylate (78a)

To a stirred mixture of CI-4 (50.09 mg, 0.221 mmol, 1 equiv) and trimethylamine 25 (67.18 mg, 0.663 mmol, 3 equiv) in dichloromethane (5 mL) were added 3-fluoro-4-{[8-isopropoxy-7-(1H-pyrazol-4-yl)-[1,2,4]triazolo[1,5-c]pyrimidin-2-yl]amino}benzenesulfonyl chloride (100 mg, 0.221 mmol, 1 equiv) at 0° C. The resulting mixture was stirred for 1 h at 25° C. Desired product could be detected by LCMS. The mixture was concentrated under vacuum. The resulting mixture was diluted with water (5 mL) and extracted with ethyl acetate (3×10 mL). The combined organic layers were dried over anhydrous sodium sulfate and concentrated under reduced pressure. The residue was purified by reversed-phase Combi-Flash, eluted with acetonitrile:water (0.5% ammonium bicarbonate) 5%-60%.This resulted in 78a (83 mg, 43.83%) as a yellow solid. LCMS (ESI, m/z): [M+H]⁺ 642.3.

### Step B. N-(4-((2,6-diazaspiro[3.5]nonan-2-yl)sulfonyl)-2-fluorophenyl)-8-isopropoxy-7-(1H-pyrazol-4-yl)-[1,2,4]triazolo[1,5-c]pyrimidin-2-amine (CI-78)

To a stirred solution of 78a (100 mg, 0.156 mmol, 1 equiv) in dichloromethane (3 mL) was added HCl (gas) in 1,4-dioxane (170.45 mg, 4.680 mmol, 30 equiv, 4 mol/L) at 0° C. The resulting mixture was stirred for 1 h at 25° C. Desired product could be detected by LCMS. The mixture was concentrated under vacuum. The resulting mixture was diluted with trimethylamine (5 mL) and extracted with ethyl acetate (3×10 mL). The combined organic layers were dried over anhydrous sodium sulfate and concentrated under reduced pressure. The residue was purified by reversed-phase Combi-Flash, eluted with acetonitrile:water (0.5% ammonium bicarbonate)=5%-60%. This resulted in CI-78 (81 mg, 72.94%) as a white solid. LCMS (ESI, m/z): [M+H]542.2.

### Intermediate 79. 1-[6-(azetidin-3-yl)-1-methylindazol-3-yl]-1,3-diazinane-2,4-dione (CI-79)

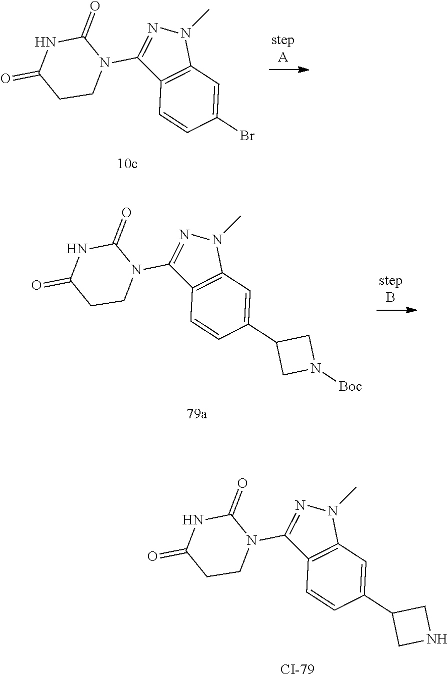 [SMILES: Cn1nc(N2CCC(=O)NC2=O)c2ccc(Br)cc21 ; Cn1nc(N2CCC(=O)NC2=O)c2ccc(C3CN(C(=O)OC(C)(C)C)C3)cc21 ; Cn1nc(N2CCC(=O)NC2=O)c2ccc(C3CNC3)cc21 ; "CI-79"]

### Step A. tert-butyl 3-(3-(2,4-dioxotetrahydropyrimidin-1(2H)-yl)-1-methyl-1H-indazol-6-yl)azetidine-1-carboxylate (79a)

To a stirred solution of 10c (1 g, 3.095 mmol, 1 equiv) in N,N-dimethylacetamide (10 mL) were added tert-butyl 3-iodoazetidine-1-carboxylate (1752.18 mg, 6.190 mmol, 2 equiv), nickel chloride (80.20 mg, 0.619 mmol, 0.2 equiv), 1H-imidazole-4-carbonitrile (57.61 mg, 0.619 mmol, 0.2 equiv), sodium iodide (231.93 mg, 1.548 mmol, 0.5 equiv), Mn (680.03 mg, 12.380 mmol, 4 equiv) and trifluoroacetic acid (352.85 mg, 3.095 mmol, 1 equiv) under nitrogen atmosphere. The resulting mixture was stirred for 12 hours at 50° C. under nitrogen atmosphere. The reaction was monitored by LCMS. Desired product could be detected by LCMS. The residue was purified by reverse flash chromatography with the following conditions: column, C18 silica gel; mobile phase, acetonitrile in water, 5% to 50% gradient in 30 min; detector, UV 254 nm. Pure fractions were evaporated to dryness to afford 79a (200 mg, 14.56%) as a white solid. LCMS (ESI, m/z): [M+H]⁺ 400.1

### Step B. 1-[6-(azetidin-3-yl)-1-methylindazol-3-yl]-1,3-diazinane-2,4-dione (CI-79)

To a stirred solution 79a (220 mg, 0.551 mmol, 1 equiv) in dichloromethane (4 mL) were added trifluoro acetic acid (0.5 mL). The resulting mixture was stirred for 1 hours at 25° C. under nitrogen atmosphere. The reaction was monitored by LCMS. Desired product could be detected by LCMS. The mixture was concentrated under reduced pressure to get 150 mg crude product and the residue was used for next step directly. LCMS (ESI, m/z): [M+H]⁺ 300.1.

### Intermediate 80. 1-{3-[4-({7-[1-(1-ethoxyethyl)pyrazol-4-yl}-8-isopropoxy-[1,2,41triazolo[1,5-c]pyrimidin-2-yl}amino)-3-fluorobenzenesulfonyl]phenyl}ethyl methanesulfonate (CI-80)

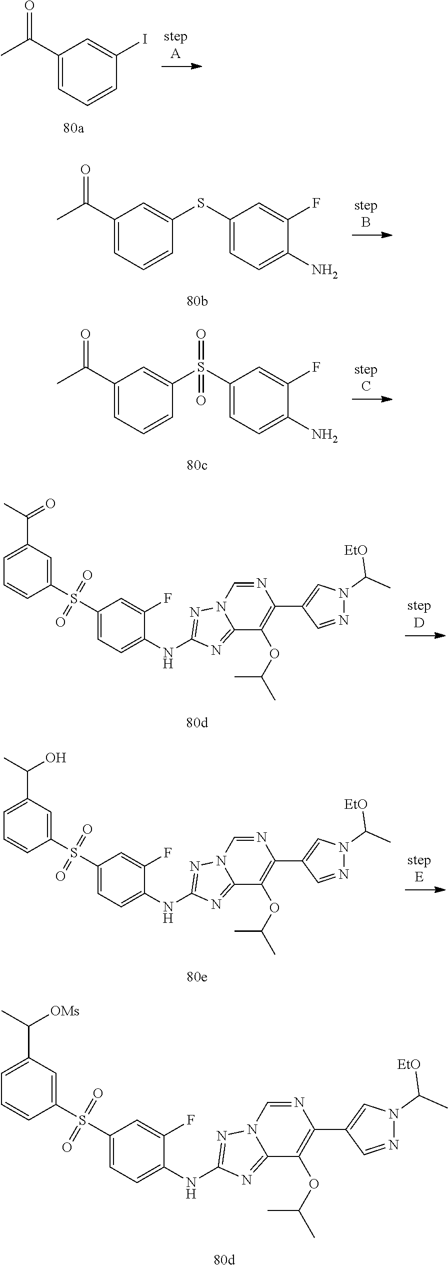

### Step A. 1-{3-[(4-amino-3-fluorophenyl)sulfanyl]phenyl}ethanone (80b)

To a stirred mixture of 80a (7 g, 28.450 mmol, 1 equiv) and 4-amino-3-fluorobenzenethiol (4.89 g, 34.140 mmol, 1.2 equiv) in 2,2,2-trifluoroethanol (70 mL) was added Pd(PPh₃)₄(3.29 g, 2.845 mmol, 0.1 equiv) and potassium phosphate tribasic (9.06 g, 42.675 mmol, 1.5 equiv). The resulting mixture was stirred for 1 h at 90° C. Desired product could be detected by LCMS. The resulting mixture was concentrated under reduced pressure. The residue was purified by silica gel column chromatography, eluted with petroleum ether/ethyl acetate (1:8) to afford 80b (7.1 g, 91.68%) as a yellow solid. LCMS (ESI, m/z): [M+H]⁺ 262.1.

### Step B. 1-[3-(4-amino-3-fluorobenzenesulfonyl)phenyl]ethanone (80c)

To a stirred mixture of 80b (6 g, 22.961 mmol, 1 equiv) in dichloromethane (60 mL) was added m-chloroperoxybenzoic acid (13.98 g, 68.883 mmol, 3 equiv, 85%). The resulting mixture was stirred for 1 h at 0° C. Desired product could be detected by LCMS. The reaction was quenched with sodium thiosulfate at 0° C. The resulting mixture was extracted with dichloromethane. The combined organic layers were dried over anhydrous sodium sulfate. After filtration, the filtrate was concentrated under reduced pressure. The residue was purified by silica gel column chromatography, eluted with petroleum ether/ethyl acetate(10:1) to afford 80c (5.2 g, 75.13%) as a white solid. LCMS (ESI, m/z): [M+H]⁺ 294.1.

### Step C. 1-{3-[4-({7-[1-(1-ethoxyethyl)pyrazol-4-yl]-8-isopropoxy-[1,2,4]triazolo[1,5-c]pyrimidin-2-yl}amino)-3-fluorobenzenesulfonyl]phenyl}ethanone (80d)

To a stirred mixture of 80c (3.56 g, 12.144 mmol, 1.2 equiv) and CI-1 (4 g, 10.120 mmol, 1.00 equiv) in dioxane (40 mL) was added Pd₂(dba)₃ (0.93 g, 1.012 mmol, 0.1 equiv) and RuPhos (0.47 g, 1.012 mmol, 0.1 equiv) and cesium carbonate (6.59 g, 20.240 mmol, 2 equiv). The resulting mixture was stirred for 1 h at 100° C. Desired product could be detected by LCMS. The resulting mixture was concentrated under reduced pressure. The residue was purified by silica gel column chromatography, eluted with petroleum ether/ethyl acetate (1:1) to afford 80d (7.9 g, 80.93%) as a white solid. LCMS (ESI, m/z): [M+H]⁺ 608.2.

### Step D. 1-{3-[4-({7-[1-(1-ethoxyethyl)pyrazol-4-yl]-8-isopropoxy-[1,2,4]triazolo[1,5-c]pyrimidin-2-yl}amino)-3-fluorobenzenesulfonyl]phenyl}ethanol (80e)

To a stirred mixture of 80d (6 g, 9.874 mmol, 1 equiv) in ethanol (60 mL) was added sodium borohydride (0.45 g, 11.849 mmol, 1.2 equiv) in portions at 0° C. The resulting mixture was stirred for 1 h at 0° C. Desired product could be detected by LCMS. The reaction was quenched by the addition of water (10 mL). The resulting mixture was concentrated under reduced pressure. The resulting mixture was extracted with EA. The combined organic layers were dried over anhydrous sodium sulfate. After filtration, the filtrate was concentrated under reduced pressure. The residue was purified by silica gel column chromatography, eluted with petroleum ether/ethyl acetate(1:1) to afford 80e (4.3 g, 60.29%) as a colorless oil. LCMS (ESI, m/z): [M+H]⁺ 610.2.

### Step E. 1-{3-[4-({7-[1-(1-ethoxyethyl)pyrazol-4-yl]-8-isopropoxy-[1,2,4]triazolo[1,5-c]pyrimidin-2-yl}amino)-3-fluorobenzenesulfonyl]phenyl}ethyl methanesulfonate (CI-80)

To a stirred mixture of 80e (1 g, 1.640 mmol, 1 equiv) in dichloromethane (10 mL) was added trimethylamine (0.66 g, 6.560 mmol, 4 equiv) and methanesulfonic anhydride (0.57 g, 3.280 mmol, 2 equiv). The resulting mixture was stirred for 1 h at 0° C. Desired product could be detected by LCMS. The reaction was quenched by the addition of water (10 mL). The resulting mixture was extracted with dichloromethane. The combined organic layers were dried over anhydrous sodium sulfate. After filtration, the filtrate was concentrated under reduced pressure to afford CI-80 (1 g, NaN) as a white solid. LCMS (ESI, m/z): [M+H]⁺ 688.2.

### Intermediate 81. 1-{1-methyl-6-[1-(piperidin-3-ylmethyl)azetidin-3-yl}indazol-3-yl}-1,3-diazinane-2,4-dione (CI-81)

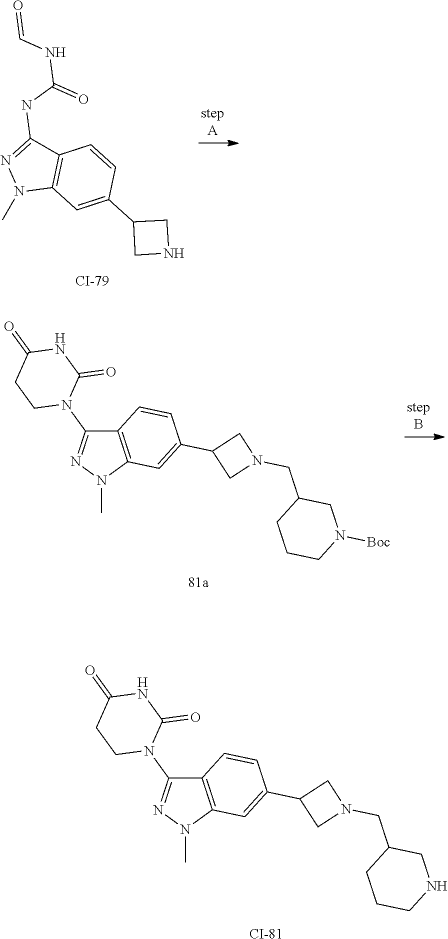 [SMILES: Cn1nc(NC(=O)NC=O)c2ccc(C3CNC3)cc21 ; "CI-79" ; Cn1nc(N2CCC(=O)NC2=O)c2ccc(C3CN(CC4CCCN(C(=O)OC(C)(C)C)C4)C3)cc21 ; Cn1nc(N2CCC(=O)NC2=O)c2ccc(C3CN(CC4CCCNC4)C3)cc21 ; "CI-81"]

### Step A. tert-butyl 3-({3-[3-(2,4-dioxo-1,3-diazinan-1-yl)-1-methylindazol-6-yl]azetidin-1-yl}methyl)piperidine-1-carboxylate (81a)

To a stirred solution of CI-79 (70 mg, 0.234 mmol, 1.00 equiv) in methanol (1 mL) were added sodium ethoxide (31.83 mg, 0.468 mmol, 2 equiv). The resulting mixture was stirred for 20 mins at 25° C. under nitrogen atmosphere. After that, tert-butyl 3-formylpiperidine-1-carboxylate (74.81 mg, 0.351 mmol, 1.5 equiv) and acetic acid (42.13 mg, 0.702 mmol, 3 equiv) was added, the resulting mixture was stirred for 0.5 h at 25° C. After that, sodium cyanoborohydride (29.39 mg, 0.468 mmol, 2 equiv) was added, the resulting mixture was stirred for 1 h at 50° C. The reaction was monitored by LCMS. Desired product could be detected by LCMS. The resulting mixture was diluted with saturated aqueous ammonium chloride (20 mL). The resulting mixture was extracted with ethyl acetate (2×15 mL). The combined organic layers were washed with brine (2×15 mL), dried over anhydrous sodium sulfate. After filtration, the filtrate was concentrated under reduced pressure. The residue was purified by reverse flash chromatography with the following conditions: column, C18 silica gel; mobile phase, acetonitrile in water, 5% to 50% gradient in 30 min; detector, UV 254 nm. Pure fractions were evaporated to dryness to afford 81a (80 mg, 65.44%) as a white solid. LCMS (ESI, m/z): [M+H]⁺ 497.2.

### Step B. 1-{1-methyl-6-[1-(piperidin-3-ylmethyl)azetidin-3-yl]indazol-3-yl}-1,3-diazinane-2,4-dione (CI-81)

To a stirred solution of 81a (70 mg, 0.141 mmol, 1 equiv) in dichloromethane (2 mL) were added HCl(gas)in 1,4-dioxane (0.5 mL). The resulting mixture was stirred for 1 hours at 25° C. under nitrogen atmosphere. The reaction was monitored by LCMS. Desired product could be detected by LCMS. The mixture was concentrated under reduced pressure to get 90 mg crude product and the residue was used for next step directly. LCMS (ESI, m/z): [M+H]⁺ 397.2.

### Intermediate 82. 1-(1-methyl-6-(5-(piperidin-3-ylmethyl)-2,5-diazabicyclo[4.1.0]heptan-2-yl)-1H-indazol-3-yl)dihydropyrimidine-2,4(1H,3H)-dione (CI-82)

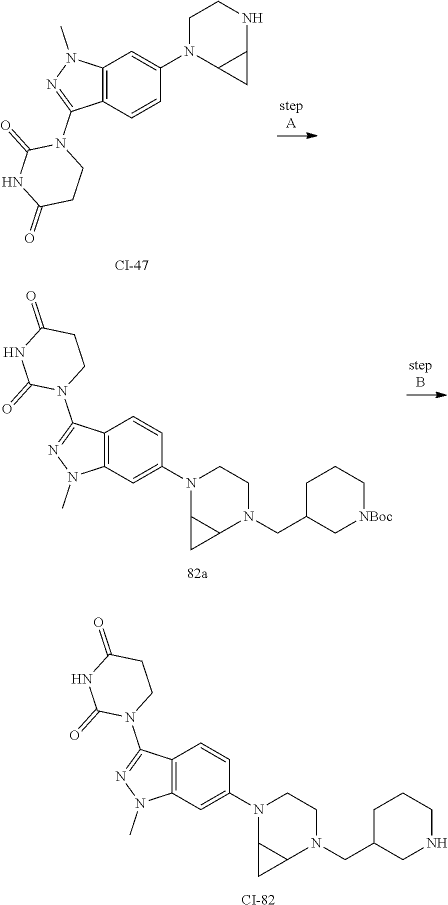

### Step A. tert-butyl 3-((5-(3-(2,4-dioxotetrahydropyrimidin-1(2H)-yl)-1-methyl-1H-indazol-6-yl)-2,5-diazabicyclo[4.1.0]heptan-2-yl)methyl)piperidine-1-carboxylate (82a)

To a stirred mixture of CI-47 (76 mg, 0.223 mmol, 1 equiv) and tert-butyl 3-formylpiperidine-1-carboxylate (57.14 mg, 0.268 mmol, 1.2 equiv) in N,N-dimethylformamide (1 mL) were added sodium cyanoborohydride (42.09 mg, 0.669 mmol, 3 equiv) and acetic acid (1.34 mg, 0.022 mmol, 0.1 equiv) in portions at 50° C. The resulting mixture was stirred for overnight at room temperature. The reaction was monitored by LCMS. The residue was purified by reversed-phase flash chromatography with the following conditions: column, C18 silica gel; mobile phase, acetonitrile in water (10 mmol/L ammonium bicarbonate), 10% to 50% gradient in 10 min; detector, UV 254 nm. This resulted in 82a (50 mg, 41.65%) as a white solid. LCMS (ESI, m/z): [M+H]⁺ 538.3.

### Step B. 1-(1-methyl-6-(5-(piperidin-3-ylmethyl)-2,5-diazabicyclo[4.1.0]heptan-2-yl)-1H-indazol-3-yl)dihydropyrimidine-2,4(1H,3H)-dione (CI-82)

A solution of 82a (40 mg, 0.091 mmol, 1 equiv) and HCl (1 mL) in dichloromethane (1 mL) was stirred for 1 h at 0° C. The reaction was monitored by LCMS. The resulting mixture was concentrated under reduced pressure. This resulted in CI-82 (40 mg) as crude product which was used in the next step directly without further purification. LCMS (ESI, m/z): [M+H]⁺ 438.2

### Intermediate 83. 1-[6-(3-{[4-(4-amino-3-fluorobenzenesulfonyl)phenyl]methyl]cyclobutyl)-1-methylindazol-3-yl}-1,3-diazinane-2,4-dione (CI-83)

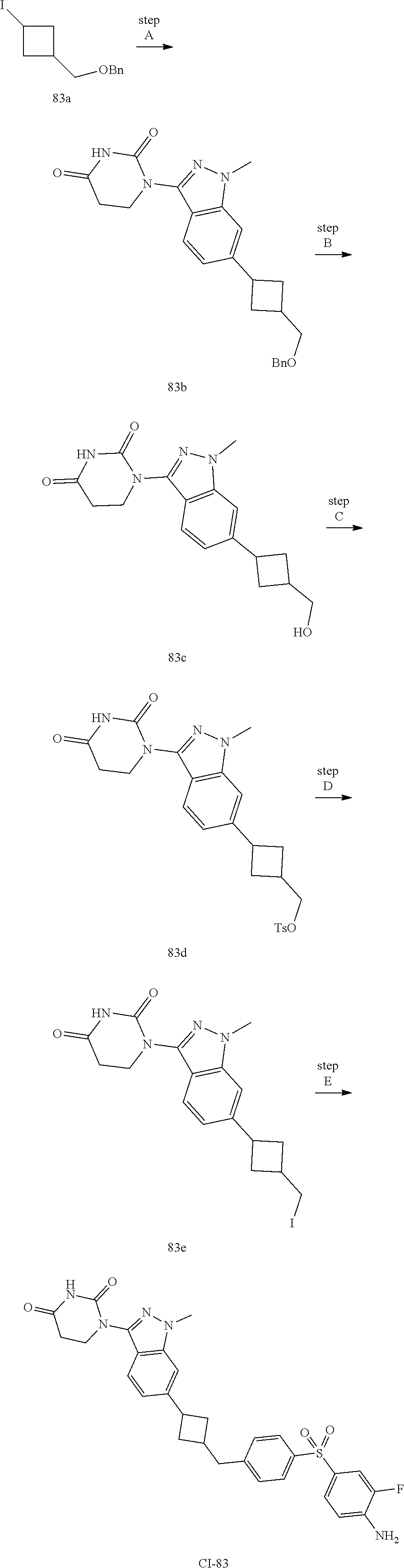

### Step A. 1-(6-{3-[(benzyloxy)methyl]cyclobutyl}-1-methylindazol-3-yl)-1,3-diazinane-2,4-dione (83b)

To a stirred solution of 10c (1 g, 3.095 mmol, 1 equiv) and 83a (1.87 g, 6.190 mmol, 2 equiv) in N,N-dimethylacetamide (12 mL) were added 1H-imidazole-4-carbonitrile (0.06 g, 0.619 mmol, 0.2 equiv) and trifluoroacetic acid (0.07 g, 0.619 mmol, 0.2 equiv) in portions at room temperature under nitrogen atmosphere. The resulting mixture was stirred for 16 h at 60° C. under nitrogen atmosphere. Desired product could be detected by LCMS. The resulting mixture was extracted with ethyl acetate (3×30 mL). The combined organic layers were washed with brine (3×30 mL), dried over anhydrous sodium sulfate. After filtration, the filtrate was concentrated under reduced pressure. The residue was purified by reversed-phase flash chromatography with the following conditions: column, C18 silica gel; mobile phase, acetonitrile in water (0.1% formic acid), 10% to 59% gradient in 30 min; detector, UV 254 nm. The resulting mixture was concentrated under reduced pressure to afford 83b (600 mg, 46.33%) as a yellow solid. LCMS (ESI, m/z): [M+H]⁺ 419.2.

### Step B. 1-{6-[3-(hydroxymethyl)cyclobutyl]-1-methylindazol-3-yl}-1,3-diazinane-2,4-dione (83c)

To a solution of 83b (600 mg, 1.434 mmol, 1 equiv) in 10 mL tetrahydrofuran was added Pd/C (10%, 250 mg) under nitrogen atmosphere in a 100 mL round-bottom flask. The mixture was hydrogenated at 50° C. for 10 h under hydrogen atmosphere using a hydrogen balloon, filtered through a Celite pad and concentrated under reduced pressure. Desired product could be detected by LCMS. The resulting mixture was concentrated under reduced pressure. The residue was purified by reversed-phase flash chromatography with the following conditions: column, C18 silica gel; mobile phase, acetonitrile in water (0.1% formic acid), 10% to 86% gradient in 30 min; detector, UV 254 nm. The resulting mixture was concentrated under reduced pressure to afford 83c (460 mg, 97.71%) as a white solid. LCMS (ESI, m/z): [M+H]⁺ 329.1.

### Step C. {3-[3-(2,4-dioxo-1,3-diazinan-1-yl)-1-methylindazol-6-yl]cyclobutyl}methyl 4-methylbenzenesulfonate (83d)

To a stirred solution of 83c (460 mg, 1.401 mmol, 1 equiv) and p-toluenesulfonyl chloride (534.12 mg, 2.802 mmol, 2 equiv) in dichloromethane (8 mL) were added triethylamine (425.27 mg, 4.203 mmol, 3 equiv) and triethylenediamine (15.71 mg, 0.140 mmol, 0.1 equiv) in portions at room temperature under nitrogen atmosphere. The resulting mixture was stirred for 2 h at room temperature under nitrogen atmosphere. Desired product could be detected by LCMS. The resulting mixture was extracted with ethyl acetate (3×20 mL). The combined organic layers were washed with brine (3×10 mL), dried over anhydrous sodium sulfate. After filtration, the filtrate was concentrated under reduced pressure. The resulting mixture was concentrated under reduced pressure. The residue was purified by silica gel column chromatography, eluted with petroleum ether/ethyl acetate(2:1) to afford 83d (450 mg, 66.57%) as a white solid. LCMS (ESI, m/z): [M+H]⁺ 483.1.

### Step D. 1-{6-[3-(iodomethyl)cyclobutyl]-1-methylindazol-3-yl}-1,3-diazinane-2,4-dione (83e)

To a stirred solution of 83d (450 mg, 0.933 mmol, 1 equiv) in acetone (5 mL) was added sodium iodide (559.12 mg, 3.732 mmol, 4 equiv) in portions at room temperature under nitrogen atmosphere. The resulting mixture was stirred for 1 h at 100° C. under nitrogen atmosphere. Desired product could be detected by LCMS. The resulting mixture was extracted with ethyl acetate (3×15 mL). The combined organic layers were washed with brine (3×10 mL), dried over anhydrous sodium sulfate. After filtration, the filtrate was concentrated under reduced pressure. The resulting mixture was concentrated under reduced pressure. The residue was purified by silica gel column chromatography, eluted with petroleum ether/ethyl acetate(2:1) to afford 83e (380 mg, 92.98%) as a white solid. LCMS (ESI, m/z): [M+H]⁺ 439.0.

### Step E. 1-[6-(3-{[4-(4-amino-3-fluorobenzenesulfonyl)phenyl]methyl}cyclobutyl)-1-methylindazol-3-yl]-1,3-diazinane-2,4-dione (CI-83)

To a stirred solution of 83e (330 mg, 0.753 mmol, 1 equiv) and CI-17 (497.21 mg, 1.506 mmol, 2 equiv) in N,N-dimethylacetamide (5 mL) were added 4,5-dihydro-3H-imidazole-4-carbonitrile (14.32 mg, 0.151 mmol, 0.2 equiv)and trifluoroacetic acid (17.17 mg, 0.151 mmol, 0.2 equiv) in portions at room temperature under nitrogen atmosphere. The resulting mixture was stirred for overnight at room temperature under nitrogen atmosphere. Desired product could be detected by LCMS. The resulting mixture was extracted with ethyl acetate (3×10 mL). The combined organic layers were washed with brine (2×10 mL), dried over anhydrous sodium sulfate. After filtration, the filtrate was concentrated under reduced pressure. The resulting mixture was concentrated under reduced pressure. The residue was purified by Prep-TLC (petroleum ether/ethyl acetatel:3) to afford CI-83 (80 mg, 18.92%) as a white solid. LCMS (ESI, m/z): [M+H]⁺ 562.1.

### Intermediate 84. N-{4-[7-(azetidin-3-ylmethyl)-2,7-diazaspiro[4.4]nonan-2-yl]sulfonyl]-2-fluorophenyl}-8-isopropoxy-7-(1H-pyrazol-4-yl)-[1,2,4]triazolo[1,5-c]pyrimidin-2-amine (CI-84)

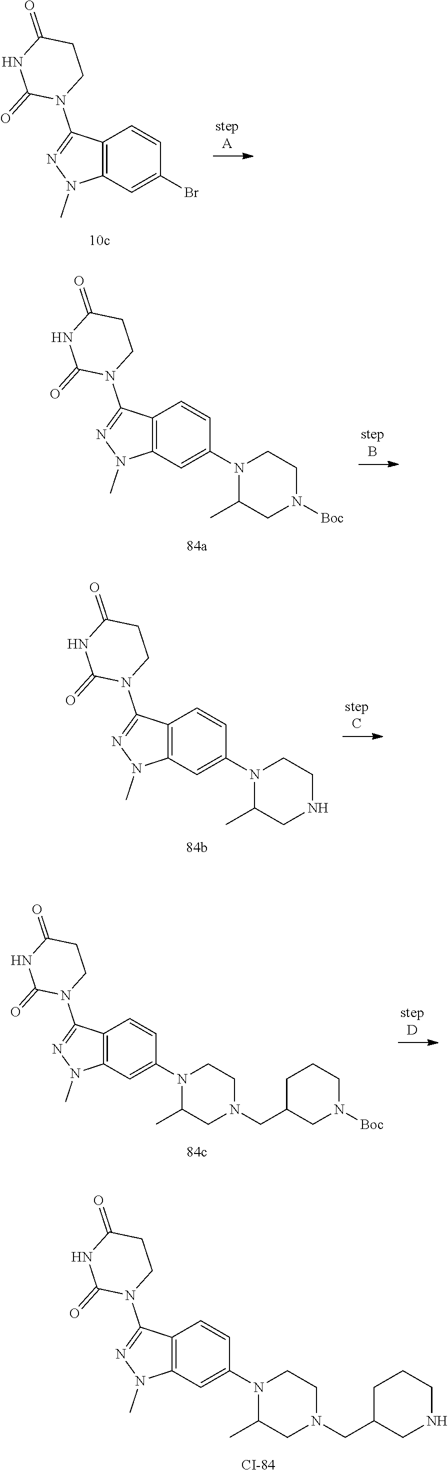

### Step A. tert-butyl 4-[3-(2,4-dioxo-1,3-diazinan-1-yl)-1-methylindazol-6-yl]-3-methylpiperazine-1-carboxylate (84a)

To a stirred mixture of 10c (500 mg, 1.547 mmol, 1 equiv) and tert-butyl 3-methylpiperazine-1-carboxylate (464.84 mg, 2.321 mmol, 1.5 equiv) in dioxane (5 mL) was added Pd-PEPPSI-IPentCl 2-methylpyridine (o-picoline) (130.15 mg, 0.155 mmol, 0.10 equiv) and sodium tert-butoxide (446.10 mg, 4.641 mmol, 3 equiv) at room temperature under nitrogen atmosphere. The resulting mixture was stirred for 2 h at 100° C. under nitrogen atmosphere. Desired product could be detected by LCMS. The resulting mixture was diluted 10 with water (100 mL). The resulting mixture was extracted with ethyl acetate (200 mL). The combined organic layers were washed with brine (100 mL), dried over anhydrous sodium sulfate. After filtration, the filtrate was concentrated under reduced pressure. The residue was purified by silica gel column chromatography, eluted with petroleum ether/ethyl acetate(1:1) to afford 84a (230 mg, 30.23%) as a light yellow solid. L CMS (ESI, m/z): [M+H]⁺ 443.2.

### Step B. 1-[1-methyl-6-(2-methylpiperazin-1-yl)indazol-3-yl]-1,3-diazinane-2,4-dione (84b)

To a stirred mixture of 84a (220 mg, 0.497 mmol, 1 equiv) in acetonitrile (10 mL) was added HCl (gas) in 1,4-dioxane (543.79 mg, 14.910 mmol, 30 equiv) dropwise at 0° C. under nitrogen atmosphere. The resulting mixture was stirred for 1 h at 0° C. under nitrogen atmosphere. Desired product could be detected by LCMS. The resulting mixture was concentrated under reduced pressure. The crude product was used in the next step directly without further purification. LCMS (ESI, m/z): [M+H]⁺ 343.2.

### Step C. tert-butyl 3-({4-[3-(2,4-dioxo-1,3-diazinan-1-yl)-1-methylindazol-6-yl]-3-methylpiperazin-1-yl}methyl)piperidine-1-carboxylate (84c)

A solution of 84b (200 mg, 0.584 mmol, 1 equiv) in N,N-dimethylformamide (5 mL) was treated with tert-butyl 3-formylpiperidine-1-carboxylate (249.15 mg, 1.168 mmol, 2 equiv) and acetic acid (175.38 mg, 2.920 mmol, 5 equiv) for 1 h at 50° C. under nitrogen atmosphere followed by the addition of sodium triacetoxyborohydride (371.39 mg, 1.752 mmol, 3 equiv) at room temperature. The resulting mixture was stirred for 2 h at room temperature under nitrogen atmosphere. Desired product could be detected by LCMS. The residue was purified by reversed-phase flash chromatography with the following conditions: column, C18 silica gel; mobile phase, acetonitrile in water (10 mmol/L ammonium bicarbonate), 40% to 50% gradient in 10 min; detector, UV 254 nm. This resulted in 84c (238 mg, 64.18%) as a yellow solid. LCMS (ESI, m/z): [M+H]540.3.

### Step D. 1-{1-methyl-6-[2-methyl-4-(piperidin-3-ylmethyl)piperazin-1-yl]indazol-3-yl}-1,3-diazinane-2,4-dione (CI-84)

A solution of 84c (200 mg, 0.371 mmol, 1 equiv) and HCl (gas) in 1,4-dioxane (135.12 mg, 3.710 mmol, 10 equiv) in acetonitrile (5 mL) was stirred for 1 h at 0° C. under nitrogen atmosphere. Desired product could be detected by LCMS. The resulting mixture was concentrated under reduced pressure. The crude product was used in the next step directly without further purification. LCMS (ESI, m/z): [M+H]⁺ 440.3.

### Intermediate 85. 1-(1-methyl-6-(3-methyl-1-(piperidin-3-ylmethyl)piperidin-4-yl)-1H-indazol-3-yl)dihydropyrimidine-2,4(1H,3H)-dione (CI-85)

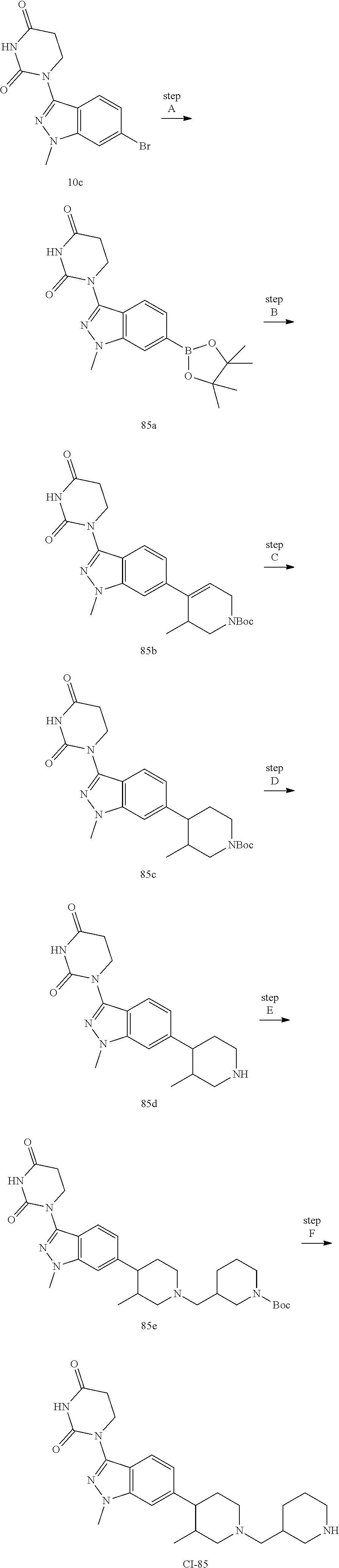

### Step A. 1-(1-methyl-6-(4,4,5,5-tetramethyl-1,3,2-dioxaborolan-2-yl)-1H-indazol-3-yl)dihydropyrimidine-2,4(1H,3H)-dione (85a)

A solution of 10c (300 mg, 0.928 mmol, 1 equiv),4,4,5,5-tetramethyl-2-(4,4,5,5-tetramethyl-1,3,2-dioxaborolan-2-yl)-1,3,2-dioxaborolane (471.49 mg, 1.856 mmol, 2 equiv), potassium acetate (182.22 mg, 1.856 mmol, 2 equiv) and Pd(dppf)C₁₋₂ (67.93 mg, 0.093 mmol, 0.1 equiv) in dioxane (5 mL) was stirred for 1 h at 80° C. under nitrogen atmosphere. The reaction was monitored by LCMS. Desired product could be detected by LCMS. The resulting mixture was washed with 3×10 mL of water. The aqueous layer was extracted with ethyl acetate (3×100 mL). The residue was purified by silica gel column chromatography, eluted with petroleum ether/ethyl acetate(5:1) to afford 85a (180 mg, 52.37%) as a white solid. LCMS (ESI, m/z): [M+H]⁺ 371.2.

### Step B. tert-butyl 4-(3-(2,4-dioxotetrahydropyrimidin-1(2H)-yl)-1-methyl-1H-indazol-6-yl)-3-methyl-3,6-dihydropyridine-1(2H)-carboxylate (85b)

To a solution of 85a (770.21 mg, 2.383 mmol, 1.1 equiv) in dioxane (12 mL) and water (3 mL) were added potassium phosphate tribasic (919.61 mg, 4.332 mmol, 2 equiv) and XPhos Pd G3 (183.36 mg, 0.217 mmol, 0.1 equiv). After stirring for 1 h at 80° C. under a nitrogen atmosphere, the reaction progress was monitored by LCMS. Desired product could be detected by LCMS. The resulting mixture was concentrated under reduced pressure. The residue was purified by Prep-TLC/silica gel column chromatography, eluted with petroleum ether/ethyl acetate(1:2) to afford 85b (800 mg, 84.03%) as a white solid. LCMS (ESI, m/z): [M+H]⁺ 440.2.

### Step C. tert-butyl 4-(3-(2,4-dioxotetrahydropyrimidin-1(2H)-yl)-1-methyl-1H-indazol-6-yl)-3-methylpiperidine-1-carboxylate (85c)

To a stirred solution of 85b (400 mg, 0.910 mmol, 1 equiv) in tetrahydrofuran (10 mL) were added Pd/C (193.70 mg, 1.820 mmol, 2 equiv). The resulting mixture was stirred for 5 hours at 25° C. under nitrogen atmosphere. The reaction was monitored by LCMS. Desired product could be detected by LCMS. After filtration, the filtrate was concentrated under reduced pressure. The mixture was concentrated under reduced pressure to get 300 mg crude product and the residue was used for next step directly. LCMS (ESI, m/z): [M+H]⁺ 442.2.

### Step D. 1-(1-methyl-6-(3-methylpiperidin-4-yl)-1H-indazol-3-yl)dihydropyrimidine-2,4(1H,3H)-dione (85d)

To a stirred solution of 85c (300 mg, 0.679 mmol, 1 equiv) in dichloromethane (6 mL) were added HCl(gas)in 1,4-dioxane (2 mL). The resulting mixture was stirred for 1 hours at 0° C. under nitrogen atmosphere. The reaction was monitored by LCMS. Desired product could be detected by LCMS. The mixture was concentrated under reduced pressure to get 350 mg crude product and the residue was used for next step directly. LCMS (ESI, m/z): [M+H]⁺ 342.2.

### Step E. tert-butyl 3-((4-(3-(2,4-dioxotetrahydropyrimidin-1(2H)-yl)-1-methyl-1H-indazol-6-yl)-3-methylpiperidin-1-yl)methyl)piperidine-1-carboxylate (85e)

To a stirred solution of 85d (70 mg, 0.205 mmol, 1 equiv) in methanol (2 mL) were added potassium acetate (40.24 mg, 0.410 mmol, 2 equiv), acetic acid (36.94 mg, 0.615 mmol, 3 equiv) and sodium cyanoborohydride (25.77 mg, 0.410 mmol, 2 equiv) in portions at 25° C. under nitrogen atmosphere. The resulting mixture was stirred for 4 hours at 50° C. under nitrogen atmosphere. The reaction was monitored by LCMS. Desired product could be detected by LCMS. The residue was purified by reverse flash chromatography with the following conditions: column, C18 silica gel; mobile phase, acetonitrile in water, 5% to 80% gradient in 30 min; detector, UV 254 nm. Pure fractions were evaporated to dryness to afford 85e (80 mg, 68.81%) as a off-white oil. LCMS (ESI, m/z): [M+H]⁺ 539.3.

### Step F. 1-(1-methyl-6-(3-methyl-1-(piperidin-3-ylmethyl)piperidin-4-yl)-1H-indazol-3-yl)dihydropyrimidine-2,4(1H,3H)-dione (CI-85)

To a stirred solution of 85e (80 mg, 0.149 mmol, 1 equiv) in acetonitrile (4 mL) was added HCl(gas)in 1,4-dioxane (2 mL) in portions at 25° C. under nitrogen atmosphere. The resulting mixture was stirred for 1 hours at 25° C. under nitrogen atmosphere. The reaction was monitored by LCMS. Desired product could be detected by LCMS. The mixture was concentrated under reduced pressure to get 90 mg crude product and the residue was used for next step directly. LCMS (ESI, m/z): [M+H]439.2.

### Intermediate 86. 1-(6-(2,2-dimethyl-1-(piperidin-3-ylmethyl) piperidin-4-yl)-1-methyl-1H-indazol-3-yl)dihydropyrimidine-2,4(1H,3H)-dione (CI-86)

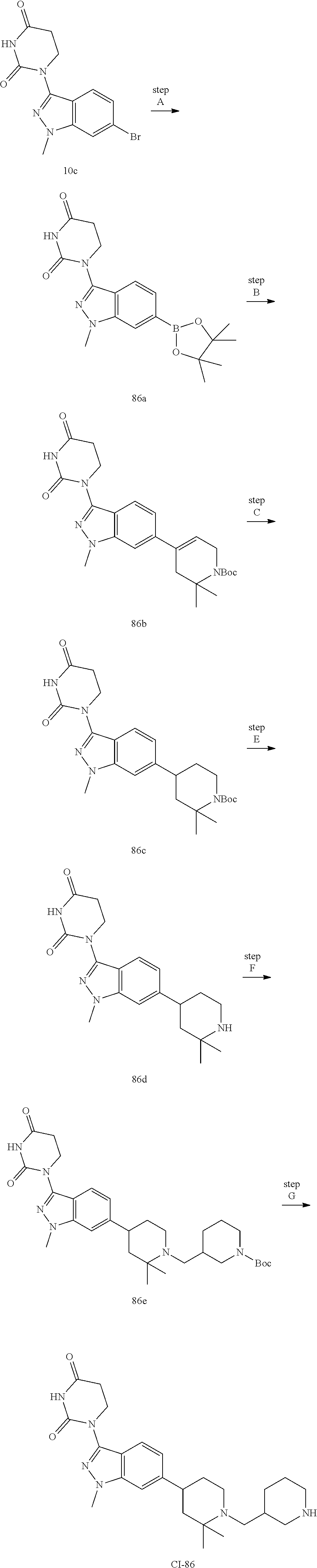

### Step A. 1-(1-methyl-6-(4,4,5,5-tetramethyl-1,3,2-dioxaborolan-2-yl)-1H-indazol-3-yl)dihydropyrimidine-2,4(1H,3H)-dione (86a)

A solution of 10c (471.49 mg, 1.856 mmol, 2 equiv), potassium acetate (182.22 mg, 1.856 mmol, 2 equiv) and Pd(dppf)C₁₋₂ (67.93 mg, 0.093 mmol, 0.1 equiv) in dioxane (5 mL) was stirred for 1 h at 80° C. under nitrogen atmosphere. The reaction was monitored by LCMS. Desired product could be detected by LCMS. The resulting mixture was washed with 3×10 mL of water. The aqueous layer was extracted with ethyl acetate (3×100 mL). The residue was purified by silica gel column chromatography, eluted with petroleum ether/ethyl acetate(5:1) to afford 86a (180 mg, 52.37%) as a white solid. LCMS (ESI, m/z): [M+H]⁺ 371.2.

### Step B. tert-butyl 4-(3-(2,4-dioxotetrahydropyrimidin-1(2H)-yl)-1-methyl-1H-indazol-6-yl)-2,2-dimethyl-3,6-dihydropyridine-1(2H)-carboxylate (86b)

To a solution of tert-butyl 2,2-dimethyl-4-(trifluoromethanesulfonyloxy)-3,6-dihydropyridine-1-carboxylate (400 mg, 1.113 mmol, 1 equiv) and 86a (494.51 mg, 1.336 mmol, 1.2 equiv) in dioxane (8 mL) and water (2 mL) were added sodium carbonate (353.92 mg, 3.339 mmol, 3 equiv) and Pd(dppf)C₁₋₂ (81.45 mg, 0.111 mmol, 0.1 equiv). After stirring for 1 at 60° C. under a nitrogen atmosphere, The reaction progress was monitored by LCMS. Desired product could be detected by LCMS. The resulting mixture was concentrated under reduced pressure. The residue was purified by Prep-TLC/silica gel column chromatography, eluted with petroleum ether/ethyl acetate(1:1) to afford 86b (300 mg, 56.45%) as a yellow solid. LCMS (ESI, m/z): [M+H]⁺ 454.2.

### Step C. tert-butyl 4-(3-(2,4-dioxotetrahydropyrimidin-1(2H)-yl)-1-methyl-1H-indazol-6-yl)-2,2-dimethylpiperidine-1-carboxylate (86c)

To a stirred solution of 86b (200 mg, 0.441 mmol, 1 equiv) in tetrahydrofuran (5 mL) were added Pd/C (140.78 mg, 1.323 mmol, 3 equiv) under hydrogen atmosphere. The resulting mixture was stirred for 3 hours at 25° C. under hydrogen atmosphere. The reaction was monitored by LCMS. Desired product could be detected by LCMS. After filtration, the filtrate was concentrated under reduced pressure. The mixture was concentrated under reduced pressure to get 180 mg crude product and the residue was used for next step directly. LCMS (ESI, m/z): [M+H]⁺ 456.2.

### Step D. 1-(6-(2,2-dimethylpiperidin-4-yl)-1-methyl-1H-indazol-3-yl)dihydropyrimidine-2,4(1H,3H)-dione (86d)

To a stirred solution of 86c (200 mg, 0.439 mmol, 1 equiv) in dichloromethane (2 mL) were added HCl(gas)in 1,4-dioxane (0.4 mL) under hydrogen atmosphere. The resulting mixture was stirred for 1 hours at 0° C. under hydrogen atmosphere. The reaction was monitored by LCMS. Desired product could be detected by LCMS. The mixture was concentrated under reduced pressure to get 86d (180 mg) as crude product which was used for next step directly. LCMS (ESI, m/z): [M+H]⁺ 336.2.

### Step E. tert-butyl 3-((4-(3-(2,4-dioxotetrahydropyrimidin-1(2H)-yl)-1-methyl-1H-indazol-6-yl)-2,2-dimethylpiperidin-1-yl)methyl) piperidine-1-carboxylate (86e)

To a stirred solution of 86d (60 mg, 0.281 mmol, 1 equiv) in metanol (2 mL) were added potassium acetate (55.22 mg, 0.562 mmol, 2 equiv) The resulting mixture was stirred for 10 mins at 25° C. under nitrogen atmosphere. To the above mixture was added sodium cyanoborohydride (35.36 mg, 0.562 mmol, 2 equiv), acetic acid (50.68 mg, 0.843 mmol, 3 equiv) and 1-[6-(2,2-dimethylpiperidin-4-yl)-1-methylindazol-3-yl]-1,3-diazinane-2,4-dione (99.99 mg, 0.281 mmol, 1 equiv) at 50° C. The resulting mixture was stirred for additional 5 hours at 50° C. The reaction was monitored by LCMS. Desired product could be detected by LCMS. The residue was purified by reverse flash chromatography with the following conditions: column, C18 silica gel; mobile phase, acetonitrile in water, 5% to 50% gradient in 30 min; detector, UV 254 nm. Pure fractions were evaporated to dryness to afford 86e (60 mg, 36.66%) as a white solid. LCMS (ESI, m/z): [M+H]⁺ 553.3.

### Step F. 1-(6-(2,2-dimethyl-1-(piperidin-3-ylmethyl) piperidin-4-yl)-1-methyl-1H-indazol-3-yl)dihydropyrimidine-2,4(1H,3H)-dione (CI-86)

To a stirred solution of 86e (60 mg, 0.109 mmol, 1 equiv) in acetonitrile (2 mL) were added HCl(gas)in 1,4-dioxane (1 mL, 0.218 mmol). The reaction was monitored by LCMS. Desired product could be detected by LCMS. The mixture was concentrated under reduced pressure to afford CI-86 (70 mg) as crude product which was used for next step directly. LCMS (ESI, m/z): [M+H]⁺ 453.3.

### Example 1. 1-(6-(1-(3-((4-((8-isopropoxy-7-(1H-pyrazol-4-yl)-[1,2,4]triazolo[1,5-c]pyrimidin-2-yl)amino)piperidin-1-yl)sulfonyl)benzyl)piperidin-4-yl)-1-methyl-1H-indazol-3-yl)dihydropyrimidine-2,4(1H,3H)-dione (1)

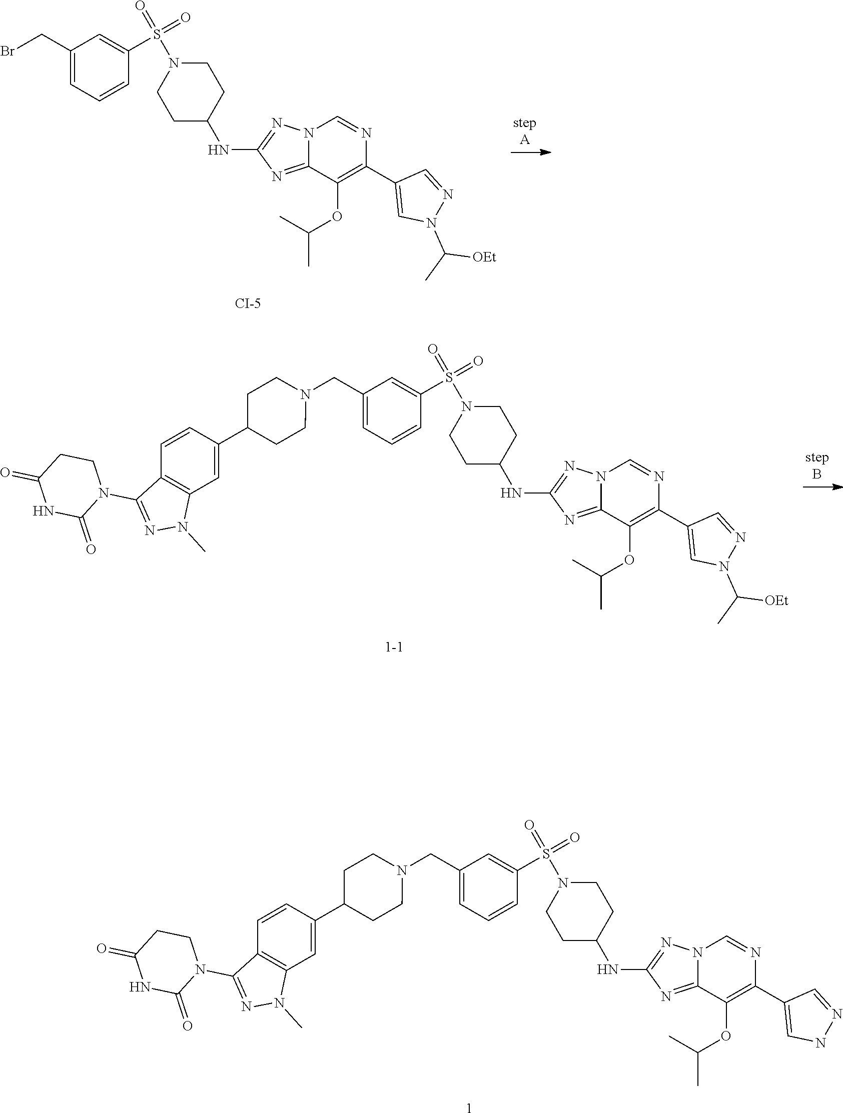 [SMILES: CCOC(C)n1cc(-c2ncn3nc(NC4CCN(S(=O)(=O)c5cccc(CBr)c5)CC4)nc3c2OC(C)C)cn1 ; CCOC(C)n1cc(-c2ncn3nc(NC4CCN(S(=O)(=O)c5cccc(CN6CCC(c7ccc8c(N9CCC(=O)NC9=O)nn(C)c8c7)CC6)c5)CC4)nc3c2OC(C)C)cn1 ; "1-1" ; CC(C)Oc1c(-c2cn[nH]c2)ncn2nc(NC3CCN(S(=O)(=O)c4cccc(CN5CCC(c6ccc7c(N8CCC(=O)NC8=O)nn(C)c7c6)CC5)c4)CC3)nc12 ; "CI-5" ; "1"]

### Step A. 1-(6-(1-(3-((4-((7-(1-(1-ethoxyethyl)-1H-pyrazol-4-yl)-8-isopropoxy-[1,2,4]triazolo[1,5-c]pyrimidin-2-yl)amino)piperidin-1-yl)sulfonyl)benzyl)piperidin-4-yl)-1-methyl-1H-indazol-3-yl)dihydropyrimidine-2,4(1H,3H)-dione (1-1)

To a stirred solution of 1-[3-(bromomethyl)benzenesulfonyl]-N-{7-[1-(1-ethoxyethyl)pyrazol-4-yl]-8-isopropoxy-[1,2,4]triazolo[1,5-c]pyrimidin-2-yl}piperidin-4-amine (CI-5, 70 mg, 0.108 mmol, 1 equiv) and 1-[1-methyl-6-(piperidin-4-yl)indazol-3-yl]-1,3-diazinane-2,4-dione (CI-10, 35.39 mg, 0.108 mmol, 1 equiv) in acetonitrile (1 mL) were added potassium carbonate (44.82 mg, 0.324 mmol, 3 equiv) and potassium iodide (8.97 mg, 0.054 mmol, 0.5 equiv) in portions at room temperature under nitrogen atmosphere. The reaction was monitored by LCMS. The residue was dissolved in ethyl acetate (5 mL). The resulting mixture was washed with water. The resulting mixture was concentrated under reduced pressure. This resulted in 1-{6-[1-({3-[4-({7-[1-(1-ethoxyethyl)pyrazol-4-yl]-8-isopropoxy-[1,2,4]triazolo[1,5-c]pyrimidin-2-yl}amino)piperidin-1-ylsulfonyl]phenyl}methyl)piperidin-4-yl]-1-methylindazol-3-yl}-1,3-diazinane-2,4-dione (1-1, 50 mg, 51.74%) as a yellow solid. LCMS (ESI, m/z): [M+H]⁺ 894.4

### Step B. 1-(6-(1-(3-((4-((8-isopropoxy-7-(1H-pyrazol-4-yl)-[1,2,4]triazolo[1,5-c]pyrimidin-2-yl)amino)piperidin-1-yl)sulfonyl)benzyl)piperidin-4-yl)-1-methyl-1H-indazol-3-yl)dihydropyrimidine-2,4(1H,3H)-dione (1)

To a stirred solution of trifluoroacetic acid (0.3 mL, 4.039 mmol, 51.59 equiv) in dichloromethane (1 mL) was added 1-{6-[1-({3-[4-({7-[1-(1-ethoxyethyl)pyrazol-4-yl]-8-isopropoxy-[1,2,4]triazolo[1,5-c]pyrimidin-2-yl}amino)piperidin-1-ylsulfonyl]phenyl}methyl)piperidin-4-yl]-1-methylindazol-3-yl}-1,3-diazinane-2,4-dione (1-1, 70 mg, 0.078 mmol, 1 equiv) in portions at 0° C. under nitrogen atmosphere. The resulting mixture was stirred for 1 h at room temperature under nitrogen atmosphere. The reaction was monitored by LCMS. The mixture was neutralized to pH 8 with saturated sodium bicarbonate (aq.). The residue was purified by reversed-phase flash chromatography with the following conditions: column, C18 silica gel; mobile phase, acetonitrile in water (10 mmol/L ammonium bicarbonate), 10% to 50% gradient in 10 min; detector, UV 254 nm. This resulted in 1-[6-(1-{[3-(4-{[8-isopropoxy-7-(1H-pyrazol-4-yl)-[1,2,4]triazolo[1,5-c]pyrimidin-2-yl]amino}piperidin-1-ylsulfonyl)phenyl]methyl}piperidin-4-yl)-1-methylindazol-3-yl]-1,3-diazinane-2,4-dione (1, 13.6 mg, 21.05%) as a white solid. LCMS (ESI, m/z): [M+H]⁺ 822.3

### Example 2. rac-(R)-3-(4-(1-((1-((4-((8-isopropoxy-7-(1H-pyrazol-4-yl)-[1,2,4]triazolo[1,5-c]pyrimidin-2-yl)amino)piperidin-1-yl)sulfonyl)piperidin-4-yl)methyl)piperidin-4-yl)-3-methyl-2-oxo-2,3-dihydro-1H-benzo[d]imidazol-1-yl)piperidine-2,6-dione (6)

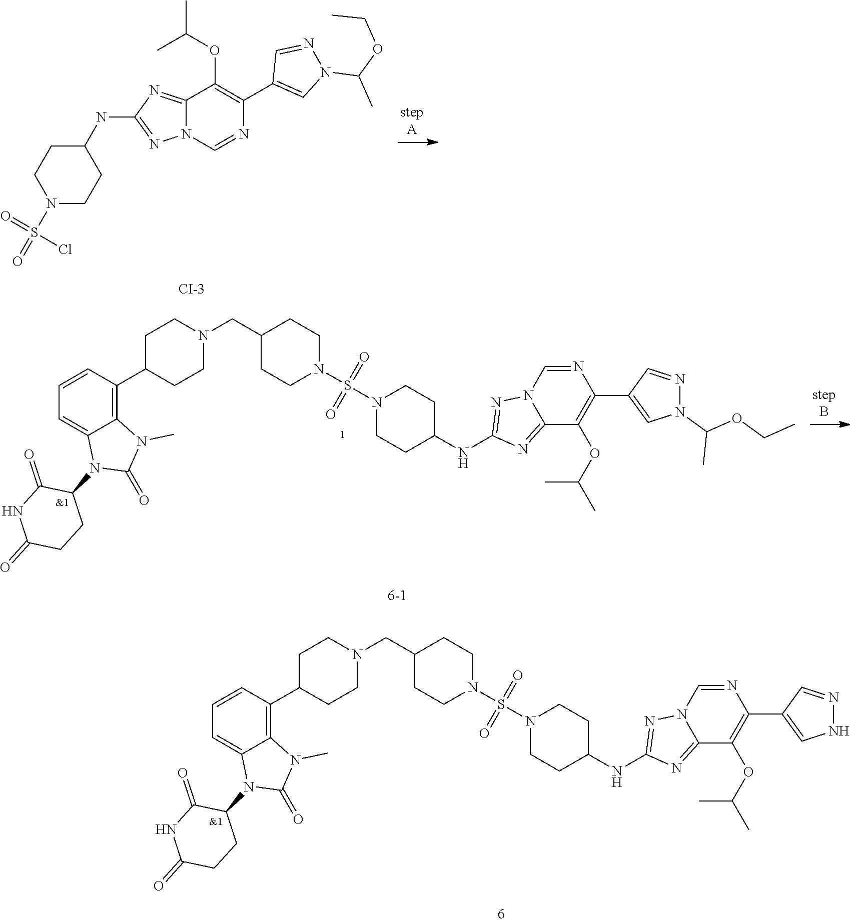

### Step A. rac-(3R)-3-(4-(1-((1-((4-((7-(1-(1-ethoxyethyl)-1H-pyrazol-4-yl)-8-isopropoxy-[1,2,4]triazolo[1,5-c]pyrimidin-2-yl)amino)piperidin-1-yl)sulfonyl)piperidin-4-yl)methyl)piperidin-4-yl)-3-methyl-2-oxo-2,3-dihydro-1H-benzo[d]imidazol-1-yl)piperidine-2,6-dione (6-1)

To a stirred solution of 4-({7-[1-(1-ethoxyethyl)pyrazol-4-yl]-8-isopropoxy-[1,2,4]triazolo[1,5-c]pyrimidin-2-yl}amino)piperidine-1-sulfonyl chloride (CI-3, 50 mg, 0.097 mmol, 1 equiv) and triethylamine (29.59 mg, 0.291 mmol, 3 equiv) in dichloromethane was added (3S)-3-{3-methyl-2-oxo-4-[1-(piperidin-4-ylmethyl)piperidin-4-yl]-1,3-benzodiazol-1-yl}piperidine-2,6-dione (CI-14, 42.84 mg, 0.097 mmol, 1 equiv) in portions at 0° C. The resulting mixture was stirred for 1 h at 0° C. under nitrogen atmosphere. The reaction was monitored by LCMS. The resulting mixture was concentrated under reduced pressure. This resulted in (3S)-3-{4-[1-({1-[4-({7-[1-(1-ethoxyethyl)pyrazol-4-yl]-8-isopropoxy-[1,2,4]triazolo[1,5-c]pyrimidin-2-yl}amino)piperidin-1-ylsulfonyl]piperidin-4-yl}methyl)piperidin-4-yl]-3-methyl-2-oxo-1,3-benzodiazol-1-yl}piperidine-2,6-dione (6-1, 50 mg, 56.00%) as a yellow solid. LCMS (ESI, m/z): [M+H]⁺ 916.4

### Step B. rac-(R)-3-(4-(1-((1-((4-((8-isopropoxy-7-(1H-pyrazol-4-yl)-[1,2,4]triazolo[1,5-c]pyrimidin-2-yl)amino)piperidin-1-yl)sulfonyl)piperidin-4-yl)methyl)piperidin-4-yl)-3-methyl-2-oxo-2,3-dihydro-1H-benzo[d]imidazol-1-yl)piperidine-2,6-dione (6)

To a stirred solution of trifluoroacetic acid (0.5 mL) in dichloromethane (1 mL) was added rac-(3R)-3-{4-[1-({1-[4-({7-[1-(1-ethoxyethyl)pyrazol-4-yl]-8-isopropoxy-[1,2,4]triazolo[1,5-c]pyrimidin-2-yl}amino) piperidin-1-ylsulfonyl]piperidin-4-yl}methyl)piperidin-4-yl]-3-methyl-2-oxo-1,3-benzodiazol-1-yl}piperidine-2,6-dione (6-1, 40 mg, 0.044 mmol, 1 equiv) in portions at room temperature under nitrogen atmosphere. The resulting mixture was stirred for 1 h at room temperature under nitrogen atmosphere. The reaction was monitored by LCMS. The reaction was monitored by LCMS. The residue was neutralized to pH 8 with saturated sodium bicarbonate (aq.). The residue was purified by reversed-phase flash chromatography with the following conditions: column, C18 silica gel; mobile phase, acetonitrile in water (10 mmol/L ammonium bicarbonate), 10% to 50% gradient in 10 min; detector, UV 254 nm. This resulted in rac-(3R)-3-[4-(1-{[1-(4-{[8-isopropoxy-7-(1H-pyrazol-4-yl)-[1,2,4]triazolo[1,5-c]pyrimidin-2-yl]amino}piperidin-1-ylsulfonyl)piperidin-4-yl]methyl}piperidin-4-yl)-3-methyl-2-oxo-1,3-benzodiazol-1-yl]piperidine-2,6-dione (6, 4.1 mg, 10.64%) as a white solid. LCMS (ESI, m/z): [M+H]⁺ 844.4

### Example 3. 1-(6-(1-(3-((3-fluoro-4-((8-isopropoxy-7-(1H-pyrazol-4-yl)-[1,2,4]triazolo[1,5-c]pyrimidin-2-yl)amino)phenyl)sulfonyl)benzyl)piperidin-4-yl)-1-methyl-1H-indazol-3-yl)dihydropyrimidine-2,4(1H,3H)-dione (8)

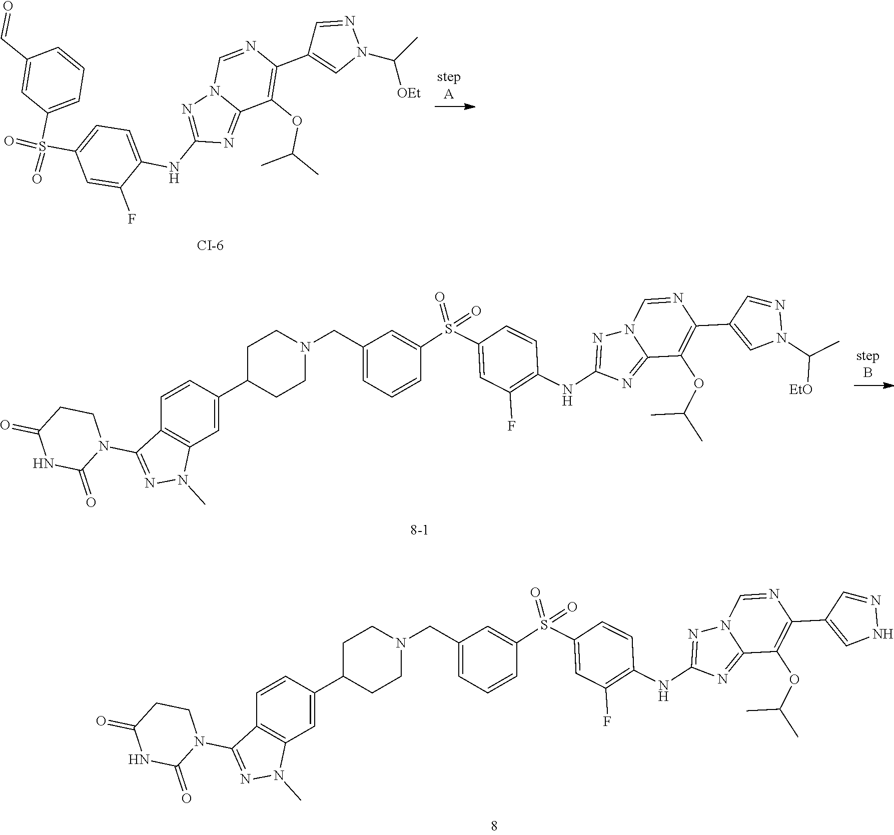 [SMILES: CCOC(C)n1cc(-c2ncn3nc(Nc4ccc(S(=O)(=O)c5cccc(C=O)c5)cc4F)nc3c2OC(C)C)cn1 ; "CI-6" ; CCOC(C)n1cc(-c2ncn3nc(Nc4ccc(S(=O)(=O)c5cccc(CN6CCC(c7ccc8c(N9CCC(=O)NC9=O)nn(C)c8c7)CC6)c5)cc4F)nc3c2OC(C)C)cn1 ; CC(C)Oc1c(-c2cn[nH]c2)ncn2nc(Nc3ccc(S(=O)(=O)c4cccc(CN5CCC(c6ccc7c(N8CCC(=O)NC8=O)nn(C)c7c6)CC5)c4)cc3F)nc12]

### Step A. 1-(6-(1-(3-((4-((7-(1-(1-ethoxyethyl)-1H-pyrazol-4-yl)-8-isopropoxy-[1,2,4]triazolo[1,5-c]pyrimidin-2-yl)amino)-3-fluorophenyl)sulfonyl)benzyl)piperidin-4-yl)-1-methyl-1H-indazol-3-yl)dihydropyrimidine-2,4(1H,3H)-dione (8-1)

To a stirred solution of 3-[4-({7-[1-(1-ethoxyethyl)pyrazol-4-yl]-8-isopropoxy-[1,2,4]triazolo[1,5-c]pyrimidin-2-yl}amino)-3-fluorobenzenesulfonyl]benzaldehyde (CI-6, 130 mg, 0.219 mmol, 1 equiv),1-[1-methyl-6-(piperidin-4-yl)indazol-3-yl]-1,3-diazinane-2,4-dione (CI-10, 86.03 mg, 0.263 mmol, 1.2 equiv), acetic acid (3.95 mg, 0.066 mmol, 0.3 equiv) in anhydrous N,N-dimethylformamide (3 mL) and stirred for 1 h at 40° C. Then sodium cyanoborohydride (41.28 mg, 0.657 mmol, 3 equiv) was added and the mixture was stirred for 1H. The reaction progress was monitored by LCMS. Desired product could be detected by LCMS. The residue was purified by silica gel column chromatography, eluted with ethyl acetate to afford tert-butyl N-{4-[3-({4-[3-(2,4-dioxo-1,3-diazinan-1-yl)-1-methylindazol-6-yl]piperidin-1-yl}methyl)benzenesulfonyl]-2-fluorophenyl}-N-{7-[1-(1-ethoxyethyl)pyrazol-4-yl]-8-isopropoxy-[1,2,4]triazolo[1,5-c]pyrimidin-2-yl}carbamate (8-1, 100 mg, 45.43%) as a colorless oil. LCMS (ESI, m/z): [M+H]⁺ 905.4

### Step B. 1-(6-(1-(3-((3-fluoro-4-((8-isopropoxy-7-(1H-pyrazol-4-yl)-[1,2,4]triazolo[1,5-c]pyrimidin-2-yl)amino)phenyl)sulfonyl)benzyl)piperidin-4-yl)-1-methyl-1H-indazol-3-yl)dihydropyrimidine-2,4(1H,3H)-dione (8)

To a stirred solution of 1-{6-[1-({3-[4-({7-[1-(1-ethoxyethyl)pyrazol-4-yl]-8-hydroxy-[1,2,4]triazolo[1,5-c]pyrimidin-2-yl}amino)-3-fluorobenzenesulfonyl]phenyl}methyl)piperidin-4-yl]-1-methylindazol-3-yl}-1,3-diazinane-2,4-dione (8-1, 95 mg, 0.110 mmol, 1 equiv) in anhydrous dichloromethane (3 mL) was added trifluoroacetic acid (1 mL) at 0° C. and stirred for 1H. The reaction progress was monitored by LCMS. Desired product could be detected by LCMS. The reaction was quenched with triethylamine at 0° C. The residue was purified by reversed-phase flash chromatography with the following conditions: column, C18 silica gel; mobile phase, 0.1% ammonium bicarbonate in acetonitrile, 40% to 60% gradient in 30 min; detector, UV 254 nm. This resulted in 1-[6-(1-{[3-(3-fluoro-4-{[8-isopropoxy-7-(1H-pyrazol-4-yl)-[1,2,4]triazolo[1,5-c]pyrimidin-2-yl]amino}benzenesulfonyl)phenyl]methyl}piperidin-4-yl)-1-methylindazol-3-yl]piperidine-2,4-dione (8, 7.3 mg, 7.73%) as a white solid. LCMS (ESI, m/z): [M+H]⁺ 833.2

### Example 4. rac-(R)-3-(4-(1-((1-((3-fluoro-4-((8-isopropoxy-7-(1H-pyrazol-4-yl)-[1,2,4]triazolo[1,5-c]pyrimidin-2-yl)amino)phenyl)sulfonyl)piperidin-4-yl)methyl)piperidin-4-yl)-3-methyl-2-oxo-2,3-dihydro-1H-benzo[d]imidazol-1-yl)piperidine-2,6-dione (14)

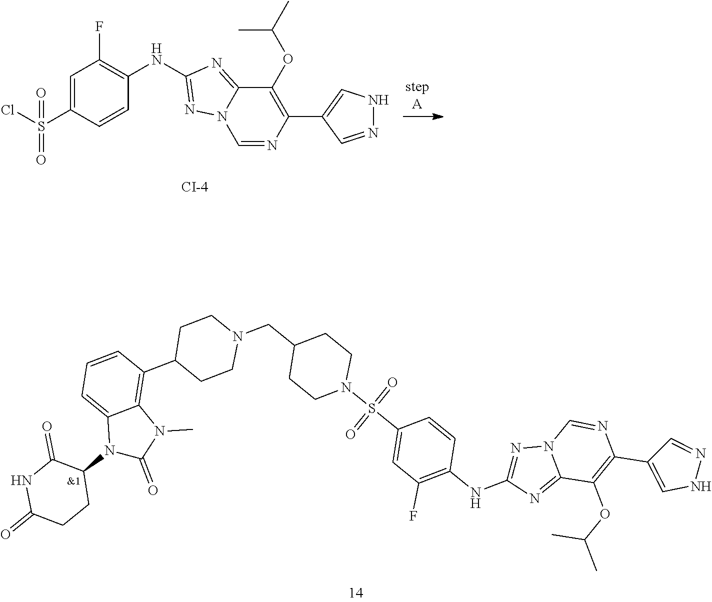 [SMILES: CC(C)Oc1c(-c2cn[nH]c2)ncn2nc(Nc3ccc(S(=O)(=O)Cl)cc3F)nc12 ; "CI-4" ; CC(C)Oc1c(-c2cn[nH]c2)ncn2nc(Nc3ccc(S(=O)(=O)N4CCC(CN5CCC(c6cccc7c6n(C)c(=O)n7[C@H]6CCC(=O)NC6=O)CC5)CC4)cc3F)nc12]

### Step A. rac-(R)-3-(4-(1-((1-((3-fluoro-4-((8-isopropoxy-7-(1H-pyrazol-4-yl)-[1,2,4]triazolo[1,5-c]pyrimidin-2-yl)amino)phenyl)sulfonyl)piperidin-4-yl)methyl)piperidin-4-yl)-3-methyl-2-oxo-2,3-dihydro-1H-benzo[d]imidazol-1-yl)piperidine-2,6-dione (14)

A solution of 4-({7-[1-(1-ethoxyethyl)pyrazol-4-yl]-8-isopropoxy-[1,2,4]triazolo[1,5-c]pyrimidin-2-yl}amino)-3-fluorobenzenesulfonyl chloride (CI-4, 100 mg, 0.191 mmol, 1 equiv) and (3S)-3-{3-methyl-2-oxo-4-[1-(piperidin-4-ylmethyl)piperidin-4-yl]-1,3-benzodiazol-1-yl}piperidine-2,6-dione (CI-14, 83.89 mg, 0.191 mmol, 1 equiv) and triethylamine (386.25 mg, 3.820 mmol, 20 equiv) in acetonitrile (8 mL) was stirred for 15 h at room temperature under nitrogen atmosphere. Mainly desired product could be detected by LCMS. The resulting mixture was concentrated under reduced pressure. The crude product (250 mg) was purified by Prep-HPLC with the following conditions (acetonitrile/water) to afford (3S)-3-[4-(1-{[1-(3-fluoro-4-{[8-isopropoxy-7-(1H-pyrazol-4-yl)-[1,2,4]triazolo[1,5-c]pyrimidin-2-yl]amino}benzenesulfonyl)piperidin-4-yl]methyl}piperidin-4-yl)-3-methyl-2-oxo-1,3-benzodiazol-1-yl]piperidine-2,6-dione (14, 35 mg, 21.00%) as a white solid. LCMS (ESI, m/z): [M+H]⁺ 855.4

### Example 5. (3S)-3-[5-(4-{1-[3-(3-fluoro-4-{[8-isopropoxy-7-(1H-pyrazol-4-yl)-[1,2,4]triazolo[1,5-c]pyrimidin-2-yl]amino}benzenesulfonyl)phenyl]azetidin-3-yl}piperazin-1-yl)-1-oxo-3H-isoindol-2-yl]piperidine-2,6-dione (21)

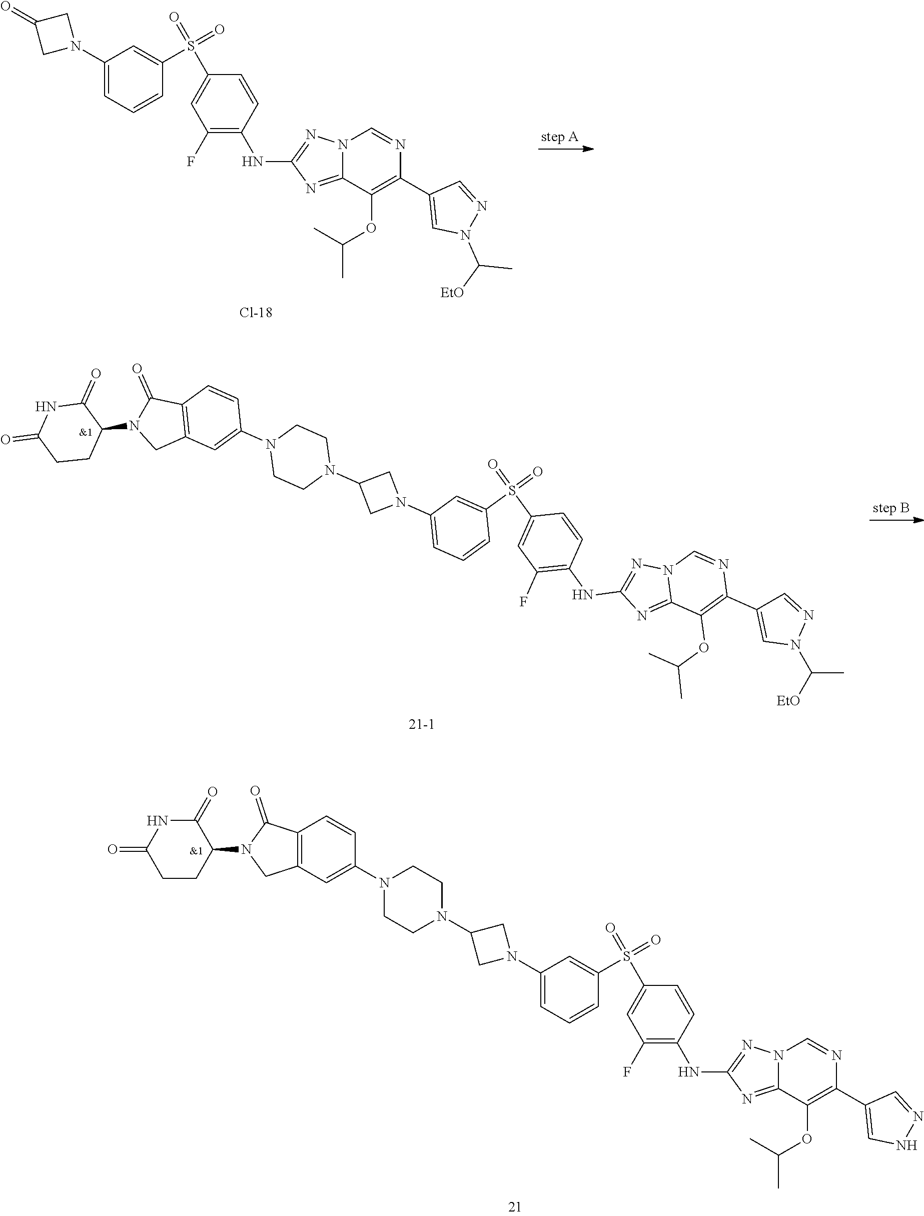

### Step A. (3S)-3-{5-[4-(1-{3-[4-({7-[1-(1-ethoxyethyl)pyrazol-4-yl]-8-isopropoxy-[1,2,4]triazolo[1,5-c]pyrimidin-2-yl}amino)-3-fluorobenzenesulfonyl]phenyl}azetidin-3-yl)piperazin-1-yl]-1-oxo-3H-isoindol-2-yl}piperidine-2,6-dione (21-1)

A solution of 1-{3-[4-({7-[1-(1-ethoxyethyl)pyrazol-4-yl]-8-isopropoxy-[1,2,4]triazolo[1,5-c]pyrimidin-2-yl}amino)-3-fluorobenzenesulfonyl]phenyl}azetidin-3-one 10 (CI-18, 120 mg, 0.189 mmol, 1 equiv) and 3-[1-oxo-5-(piperazin-1-yl)-3H-isoindol-2-yl]piperidine-2,6-dione (CI-16, 62.08 mg, 0.189 mmol, 1 equiv) in N,N-dimethylformamide (4 mL) was stirred for 30 min at 50° C. under nitrogen atmosphere. To the above mixture was added sodium triacetoxyborohydride (160.28 mg, 0.756 mmol, 4 equiv) over 15 min at room temperature. The resulting mixture was stirred for additional 2 h at room temperature. Desired product could be detected by LCMS. The residue was purified by reversed-phase flash chromatography with the following conditions: column, C18 silica gel; mobile phase, acetonitrile in water (10 mmol/L ammonium bicarbonate), 50% to 60% gradient in 10 min; detector, UV 254 nm. This resulted in (3S)-3-{5-[4-(1-{3-[4-({7-[1-(1-ethoxyethyl) pyrazol-4-yl]-8-isopropoxy-[1,2,4]triazolo[1,5-c]pyrimidin-2-yl}amino)-3-fluorobenzenesulfonyl]phenyl}azetidin-3-yl)piperazin-1-yl]-1-oxo-3H-isoindol-2-yl}piperidine-2,6-dione (21-1, 60 mg, 30.16%) as an off-white solid. LCMS (ESI, m/z): [M+H]⁺ 947.0.

### Step B. (3S)-3-[5-(4-{1-[3-(3-fluoro-4-{[8-isopropoxy-7-(1H-pyrazol-4-yl)-[1,2,4]triazolo[1,5-c]pyrimidin-2-yl]amino}benzenesulfonyl)phenyl]azetidin-3-yl}piperazin-1-yl)-1-oxo-3H-isoindol-2-yl]piperidine-2,6-dione (21)

To a stirred mixture of (3S)-3-{5-[4-(1-{3-[4-({7-[1-(1-ethoxyethyl) pyrazol-4-yl]-8-isopropoxy-[1,2,4]triazolo[1,5-c]pyrimidin-2-yl}amino)-3-fluorobenzenesulfonyl]phenyl}azetidin-3-yl)piperazin-1-yl]-1-oxo-3H-isoindol-2-yl}piperidine-2,6-dione (21-1, 120 mg, 0.127 mmol, 1 equiv) in dichloromethane (5 mL) was added trifluoroacetic acid (1 mL) dropwise at 0° C. under nitrogen atmosphere. The resulting mixture was stirred for 30 min at 0° C. under nitrogen atmosphere. Desired product could be detected by LCMS. The resulting mixture was concentrated under reduced pressure. The resulting mixture was diluted with dichloromethane (20 mL). The mixture was neutralized to pH 8 with trimethylamine. The resulting mixture was concentrated under reduced pressure. The residue was purified by reversed-phase flash chromatography with the following conditions: column, C18 silica gel; mobile phase, acetonitrile/isopropanol in water (10 mmol/L ammonium bicarbonate), 45% to 56% gradient in 10 min; detector, UV 254 nm. This resulted in (3S)-3-[5-(4-{1-[3-(3-fluoro-4-{[8-isopropoxy-7-(1H-pyrazol-4-yl)-[1,2,4]triazolo[1,5-c]pyrimidin-2-yl]amino}benzenesulfonyl)phenyl]azetidin-3-yl}piperazin-1-yl)-1-oxo-3H-isoindol-2-yl]piperidine-2,6-dione (21, 30 mg, 26.47%) as a white solid. LCMS (ESI, m/z): [M+H]⁺ 875.3.

### Example 6. (3S)-3-[5-(3-{[3-(3-fluoro-4-{[8-isopropoxy-7-(1H-pyrazol-4-yl)-[1,2,4]triazolo[1,5-c]pyrimidin-2-yl]amino}benzenesulfonyl)phenyl]methyl}cyclobutyl)-3-methyl-2-oxo-1,3-benzodiazol-1-yl]piperidine-2,6-dione (31)

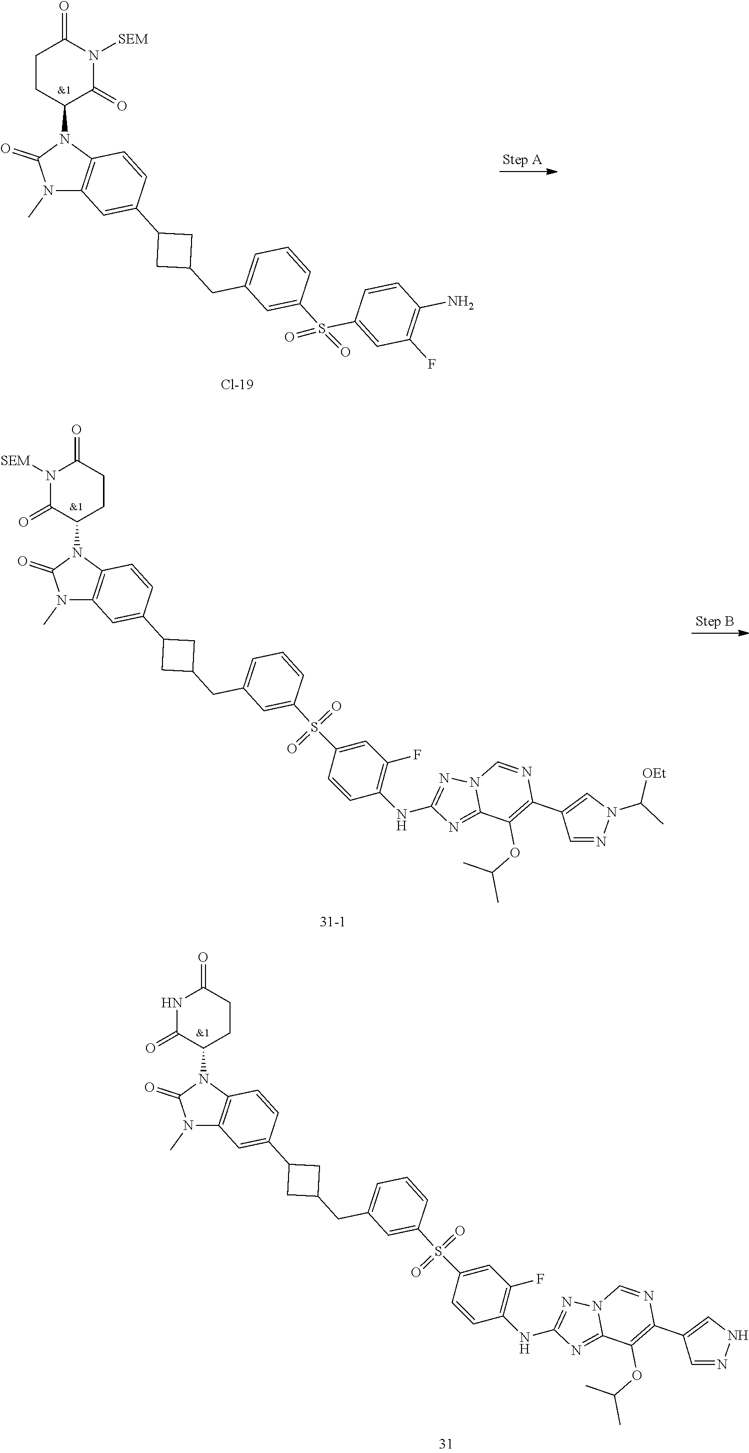

### Step A. 1-[1-methyl-6-(1-{[3-(4-{7′-[(1R,3R)-3-(oxan-2-yloxy)cyclohexyl]-6′-oxospiro[cyclopropane-1,5′-pyrrolo[2,3-d]pyrimidin]-2′-ylamino}benzenesulfonyl)phenyl]methyl}piperidin-4-yl)indazol-3-yl]-1,3-diazinane-2,4-dione (31-1)

A solution of (3S)-3-[5-(3-{[3-(4-amino-3-fluorobenzenesulfonyl)phenyl]methyl}cyclobutyl)-3-methyl-2-oxo-1,3-benzodiazol-1-yl]-1-{[2-(trimethylsilyl)ethoxy]methyl}piperidine-2,6-dione (CI-19, 100 mg, 0.141 mmol, 1 equiv), 2-bromo-7-(1-(1-ethoxyethyl)-1H-pyrazol-4-yl)-8-isopropoxy-[1,2,4]triazolo[1,5-c]pyrimidine (CI-1, 67 mg, 0.169 mmol, 1.2 equiv), Pd₂(dba)₃ (12.95 mg, 0.014 mmol, 0.1 equiv), SPhos (6.60 mg, 0.014 mmol, 0.1 equiv) and cesium carbonate (92.18 mg, 0.282 mmol, 2 equiv) in dioxane (4 mL) was stirred for 4 h at 90° C. under argon atmosphere. Desired product could be detected by LCMS. The resulting mixture was cooled to room temperature. The solvent was removed under reduced pressure. The residue was purified by silica gel column chromatography, eluted with petroleum ether in ethyl acetate from 0% to 50%. Pure fractions were evaporated to dryness to afford (3S)-3-{5-[3-({3-[4-({7-[1-(1-ethoxyethyl) pyrazol-4-yl]-8-isopropoxy-[1,2,4]triazolo[1,5-c]pyrimidin-2-yl}amino)-3-fluorobenzenesulfonyl]phenyl}methyl)cyclobutyl]-3-methyl-2-oxo-1,3-benzodiazol-1-yl}-1-{[2-(trimethylsilyl)ethoxy]methyl}piperidine-2,6-dione (31-1, 60 mg, 47.26%) as a yellow solid. LCMS (ESI, m/z): [M+H]⁺ 1021.41.

### Step B. (3S)-3-[5-(3-{[3-(3-fluoro-4-{[8-isopropoxy-7-(1H-pyrazol-4-yl)-[1,2,4]triazolo[1,5-c]pyrimidin-2-yl]amino}benzenesulfonyl)phenyl]methyl}cyclobutyl)-3-methyl-2-oxo-1,3-benzodiazol-1-yl]piperidine-2,6-dione (31)

A solution of (3S)-3-{5-[3-({3-[4-({7-[1-(1-ethoxyethyl)pyrazol-4-yl]-8-isopropoxy-[1,2,4]triazolo[1,5-c]pyrimidin-2-yl}amino)-3-fluorobenzenesulfonyl]phenyl}methyl)cyclobutyl]-3-methyl-2-oxo-1,3-benzodiazol-1-yl}-1-{[2-(trimethylsilyl)ethoxy]methyl}piperidine-2,6-dione (31-1, 30 mg, 0.029 mmol, 1 equiv) and HCl (0.54 mg, 0.015 mmol, 3 equiv) in dichloromethane (2 mL) was stirred for 30 min at 0° C. under argon atmosphere. Desired product could be detected by LCMS. The residue was purified by reversed-phase flash chromatography with the following conditions: column, C18 silica gel; mobile phase, acetonitrile in water (10 mmol/L ammonium bicarbonate), 10% to 50% gradient in 10 min; detector, UV 254 nm to afford (3S)-3-[5-(3-{[3-(3-fluoro-4-{[8-isopropoxy-7-(1H-pyrazol-4-yl)-[1,2,4]triazolo[1,5-c]pyrimidin-2-yl]amino}benzenesulfonyl)phenyl]methyl}cyclobutyl)-3-methyl-2-oxo-1,3-benzodiazol-1-yl]piperidine-2,6-dione (31, 4.2 mg, 15.98%) as a white solid. LCMS (ESI, m/z): [M+H]⁺ 819.35.

### Example 7. 3-(5-{2-[3-(3-fluoro-4-{[8-isopropoxy-7-(1H-pyrazol-4-yl)-[1,2,4]triazolo[1,5-c]pyrimidin-2-yl]amino}benzenesulfonyl)phenyl]-2,6-diazabicyclo[3.2.0]heptan-6-yl}-1-oxo-3H-isoindol-2-yl)piperidine-2,6-dione (34)

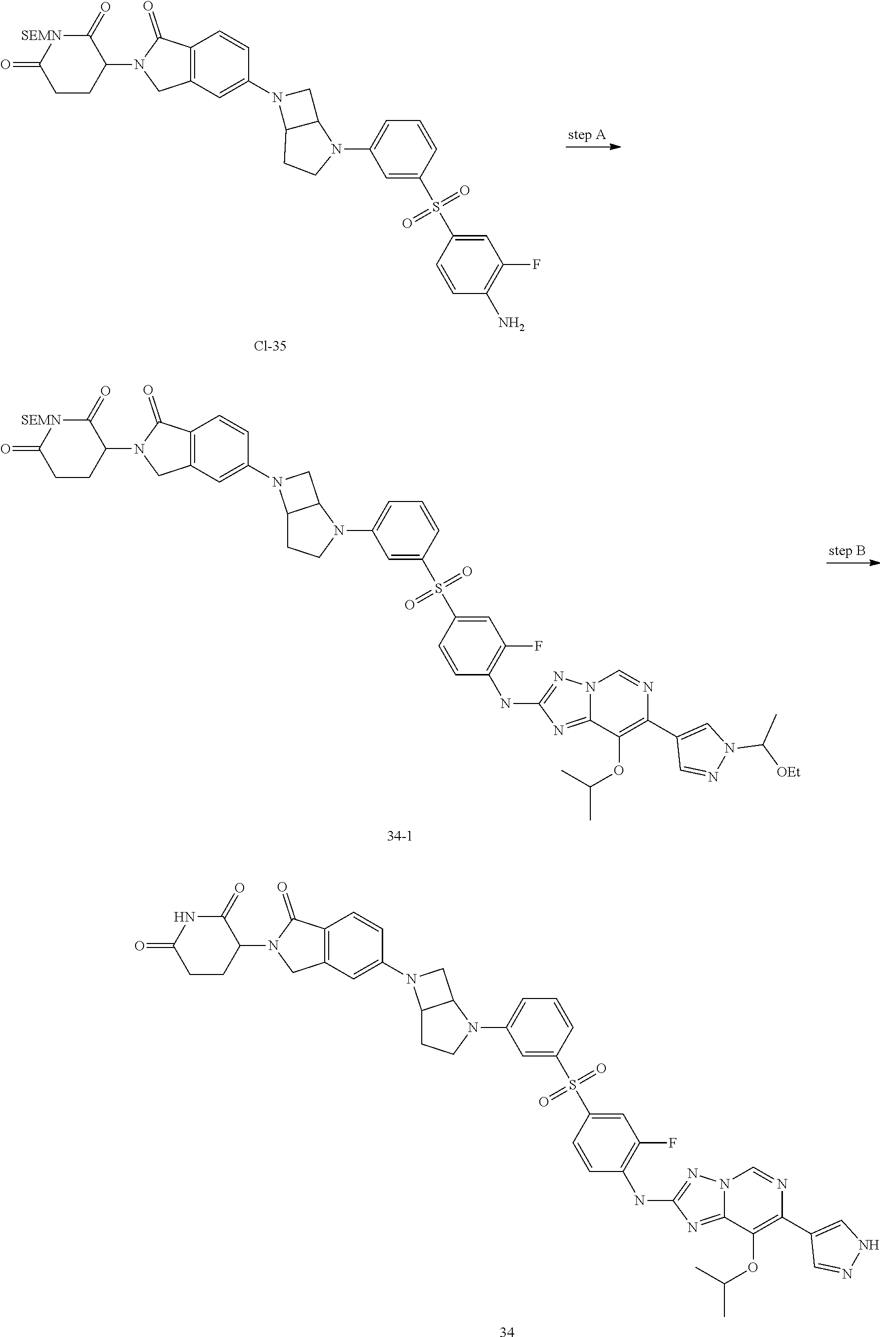

### Step A. 3-[5-(2-{3-[4-({7-[1-(1-Ethoxyethyl)pyrazol-4-yl]-8-isopropoxy-[1,2,4]triazolo[1,5-c]pyrimidin-2-yl}amino)-3-fluorobenzenesulfonyl]phenyl}-2,6-diazabicyclo[3.2.0]heptan-6-yl)-1-oxo-3H-isoindol-2-yl]-1-{[2-(trimethylsilyl)ethoxy]methyl}piperidine-2,6-dione (34-1)

A solution of 3-(5-{2-[3-(4-amino-3-fluorobenzenesulfonyl)phenyl]-2,6-diazabicyclo[3.2.0]heptan-6-yl}-1-oxo-3H-isoindol-2-yl)-1-{[2-(trimethylsilyl)ethoxy]methyl}piperidine-2,6-dione (CI-35, 60 mg, 0.083 mmol, 1 equiv) and 4-{2-bromo-8-isopropoxy-[1,2,4]triazolo[1,5-c]pyrimidin-7-yl}-1-(1-ethoxyethyl)pyrazole (CI-1, 39.53 mg, 0.100 mmol, 1.2 equiv), Ruphos Pd G4 (14.18 mg, 0.017 mmol, 0.2 equiv), Ruphos (7.78 mg, 0.017 mmol, 0.2 equiv) and cesium carbonate (54.31 mg, 0.166 mmol, 2 equiv) in dioxane (2 mL) was stirred for 1 h at 80° C. under argon atmosphere. Desired product could be detected by LCMS. The resulting mixture was diluted with water. The resulting mixture was extracted with ethyl acetate. The combined organic layers were dried over anhydrous sodium sulfate. After filtration, the filtrate was concentrated under reduced pressure. The residue was purified by Prep-TLC (dichloromethane/methanol 20:1) to afford 3-[5-(2-{3-[4-({7-[1-(1-ethoxyethyl) pyrazol-4-yl]-8-isopropoxy-[1,2,4]triazolo[1,5-c]pyrimidin-2-yl}amino)-3-fluorobenzenesulfonyl]phenyl}-2,6-diazabicyclo[3.2.0]heptan-6-yl)-1-oxo-3H-isoindol-2-yl]-1-{[2-(trimethylsilyl)ethoxy]methyl}piperidine-2,6-dione (34-1, 70 mg, 81.21%) as an off-white semi-solid. LCMS (ESI, m/z): [M+H]⁺ 1034.3.

### Step B. 3-(5-{2-[3-(3-fluoro-4-{[8-isopropoxy-7-(1H-pyrazol-4-yl)-[1,2,4]triazolo[1,5-c]pyrimidin-2-yl]amino}benzenesulfonyl)phenyl]-2,6-diazabicyclo[3.2.0]heptan-6-yl}-1-oxo-3H-isoindol-2-yl)piperidine-2,6-dione (34)

To a solution of 3-[5-(2-{3-[4-({7-[1-(1-ethoxyethyl)pyrazol-4-yl]-8-isopropoxy-[1,2,4]triazolo[1,5-c]pyrimidin-2-yl}amino)-3-fluorobenzenesulfonyl]phenyl}-2,6-diazabicyclo[3.2.0]heptan-6-yl)-1-oxo-3H-isoindol-2-yl]-1-{[2-(trimethylsilyl)ethoxy]methyl}piperidine-2,6-dione (34-1, 80 mg, 0.077 mmol, 1 equiv) in dichloromethane (2 mL) was added trifluoroacetic acid (0.5 mL, 6.730 mmol, 87.4 equiv) at 0° C. The mixture was stirred for 30 min. Then to the above mixture was added triethylamine (1362.0 mg, 13.46 mmol, 174.8 equiv) and tetramethylethylenediamine (10.18 mg, 0.115 mmol, 1.5 equiv) at 0° C. The resulting mixture was stirred for additional 30 min at room temperature. Desired product could be detected by LCMS. The resulting mixture was concentrated under reduced pressure. The residue was purified by reversed-phase flash chromatography to afford 3-(5-{2-[3-(3-fluoro-4-{[8-isopropoxy-7-(1H-pyrazol-4-yl)-[1,2,4]triazolo[1,5-c]pyrimidin-2-yl]amino}benzenesulfonyl)phenyl]-2,6-diazabicyclo[3.2.0]heptan-6-yl}-1-oxo-3H-isoindol-2-yl)piperidine-2,6-dione (34, 16.5 mg, 25.13%) as white solid. LCMS (ESI, m/z): [M+H]⁺ 832.2.

### Example 8. rac-(R)-N-(3-(4-(1-(2,6-dioxopiperidin-3-yl)-3-methyl-2-oxo-2,3-dihydro-1H-benzo[d]imidazol-5-yl)piperidin-1-yl)propyl)-3-fluoro-4-((8-isopropoxy-7-(1H-pyrazol-4-yl)-[1,2,4]triazolo[1,5-c]pyrimidin-2-yl)amino)-N,2-dimethylbenzenesulfonamide (55)

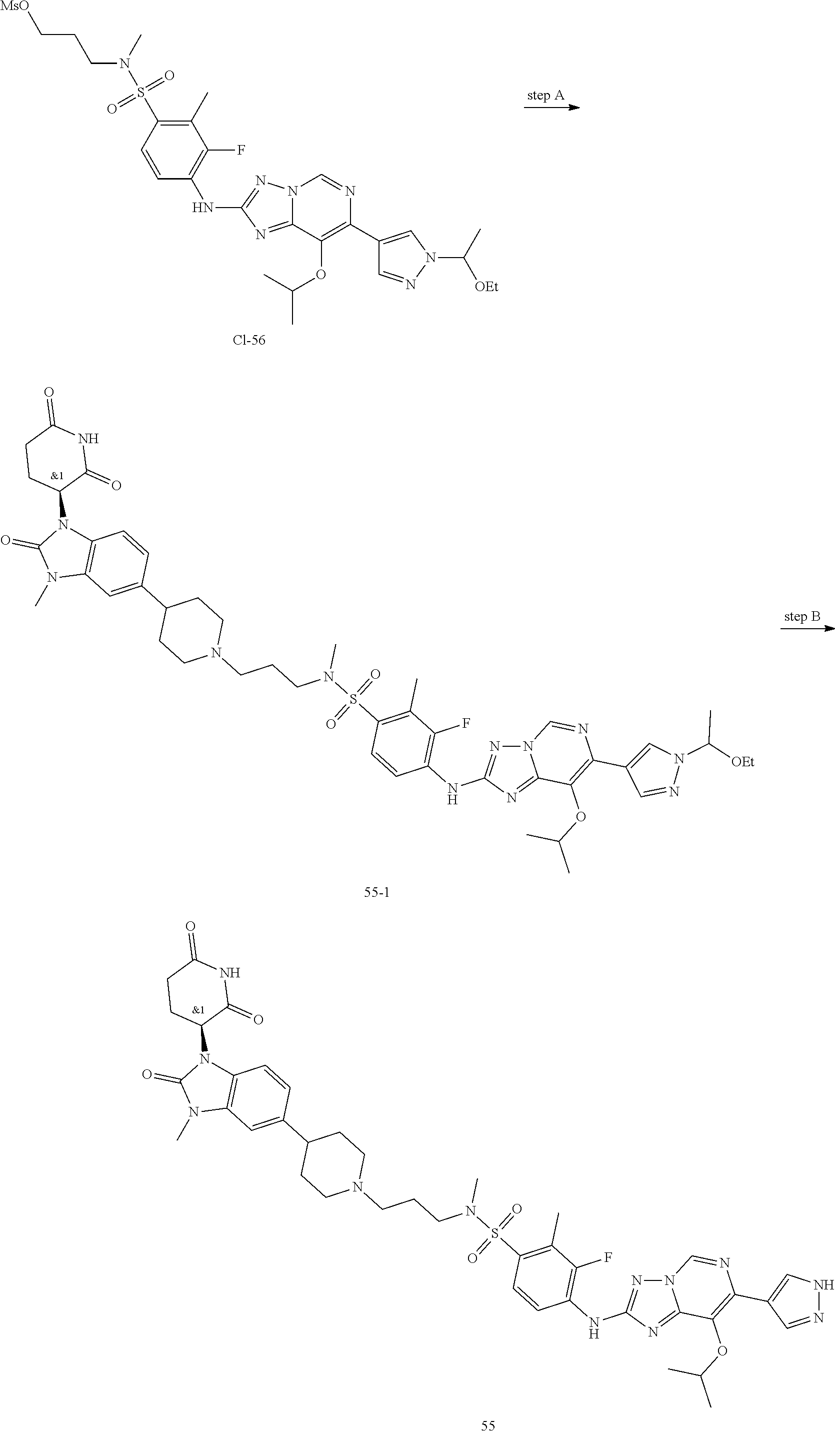

### Step A. rac-N-(3-(4-(1-((R)-2,6-dioxopiperidin-3-yl)-3-methyl-2-oxo-2,3-dihydro-1H-benzo[d]imidazol-5-yl)piperidin-1-yl)propyl)-4-((7-(1-(1-ethoxyethyl)-1H-pyrazol-4-yl)-8-isopropoxy-[1,2,4]triazolo[1,5-c]pyrimidin-2-yl)amino)-3-fluoro-N,2-dimethylbenzenesulfonamide (55-1)

To a stirred mixture of 3-[N-methyl4-({7-[1-(1-ethoxyethyl)pyrazol-4-yl]-8-isopropoxy-[1,2,4]triazolo[1,5-c]pyrimidin-2-yl}amino)-3-fluoro-2-methylbenzenesulfonamido]propyl methanesulfonate (CI-56, 60 mg, 0.090 mmol, 1 equiv) and rac-(3R)-3-[3-methyl-2-oxo-5-(piperidin-4-yl)-1,3-benzodiazol-1-yl]piperidine-2,6-dione (CI-29, 30.72 mg, 0.090 mmol, 1 equiv) in Acetone (5 mL) were added potassium carbonate (37.20 mg, 0.270 mmol, 3 equiv) and potassium iodide (14.89 mg, 0.090 mmol, 1 equiv) at 25° C. The resulting mixture was stirred for 12 h at 60° C. Desired product could be detected by LCMS. The mixture was concentrated under vacuum. The resulting mixture was diluted with water (5 mL) and extracted with ethyl acetate (3×10 mL). The combined organic layers were dried over anhydrous sodium sulfate and concentrated under reduced pressure. The residue was purified by reversed-phase Combi-Flash, eluted with acetonitrile:water (0.5% ammonium bicarbonate)=5%-60%.This resulted in rac-N-[3-(4-{1-[(3R)-2,6-dioxopiperidin-3-yl]-3-methyl-2-oxo-1,3-benzodiazol-5-yl}piperidin-1-yl)propyl]-4-({7-[1-(1-ethoxyethyl)pyrazol-4-yl]-8-isopropoxy-[1,2,4]triazolo[1,5-c]pyrimidin-2-yl}amino)-3-fluoro-N,2-dimethylbenzenesulfonamide (55-1, 40 mg, 43.85%) as a yellow solid. LCMS (ESI, m/z): [M+H]⁺ 915.4.

### Step B. rac-(R)-N-(3-(4-(1-(2,6-dioxopiperidin-3-yl)-3-methyl-2-oxo-2,3-dihydro-1H-benzo[d]imidazol-5-yl)piperidin-1-yl)propyl)-3-fluoro-4-((8-isopropoxy-7-(1H-pyrazol-4-yl)-[1,2,4]triazolo[1,5-c]pyrimidin-2-yl)amino)-N,2-dimethylbenzenesulfonamide (55)

To a stirred solution of N-[3-(4-{1-[(3S)-2,6-dioxopiperidin-3-yl]-3-methyl-2-oxo-1,3-benzodiazol-5-yl}piperidin-1-yl)propyl]-4-({7-[1-(1-ethoxyethyl)pyrazol-4-yl]-8-isopropoxy-[1,2,4]triazolo[1,5-c]pyrimidin-2-yl}amino)-3-fluoro-N,2-dimethylbenzenesulfonamide (55-1, 150 mg, 0.164 mmol, 1 equiv) in dichloromethane (5 mL) was added HCl (gas) in 1,4-dioxane (179.30 mg, 4.920 mmol, 30 equiv) at 0° C. The resulting mixture was stirred for 1 h at 0° C. Desired product could be detected by LCMS. The mixture was concentrated under vacuum. The resulting mixture was diluted with water (5 mL) and extracted with ethyl acetate (3×10 mL). The combined organic layers were dried over anhydrous sodium sulfate and concentrated under reduced pressure. The residue was purified by reversed-phase Combi-Flash, eluted with acetonitrile in water (0.5% ammonium bicarbonate) from 5% to 60%. This resulted in N-[3-(4-{1-[(3S)-2,6-dioxopiperidin-3-yl]-3-methyl-2-oxo-1,3-benzodiazol-5-yl}piperidin-1-yl)propyl]-3-fluoro-4-{[8-isopropoxy-7-(1H-pyrazol-4-yl)-[1,2,4]triazolo[1,5-c]pyrimidin-2-yl]amino}-N,2-dimethylbenzenesulfonamide (55, 37.6 mg, 26.56%) as a white solid. LCMS (ESI, m/z): [M+H]⁺ 843.3.

### Example 9. 1-(6-{4-[(1S)-1-[3-(3-fluoro-4-{[8-isopropoxy-7-(1H-pyrazol-4-yl)-[1,2,4]triazolo[1,5-c]pyrimidin-2-yl]amino}benzenesulfonyl)phenyl]ethyl]piperazin-1-yl}-1-methylindazol-3-yl)-1,3-diazinane-2,4-dione (84)

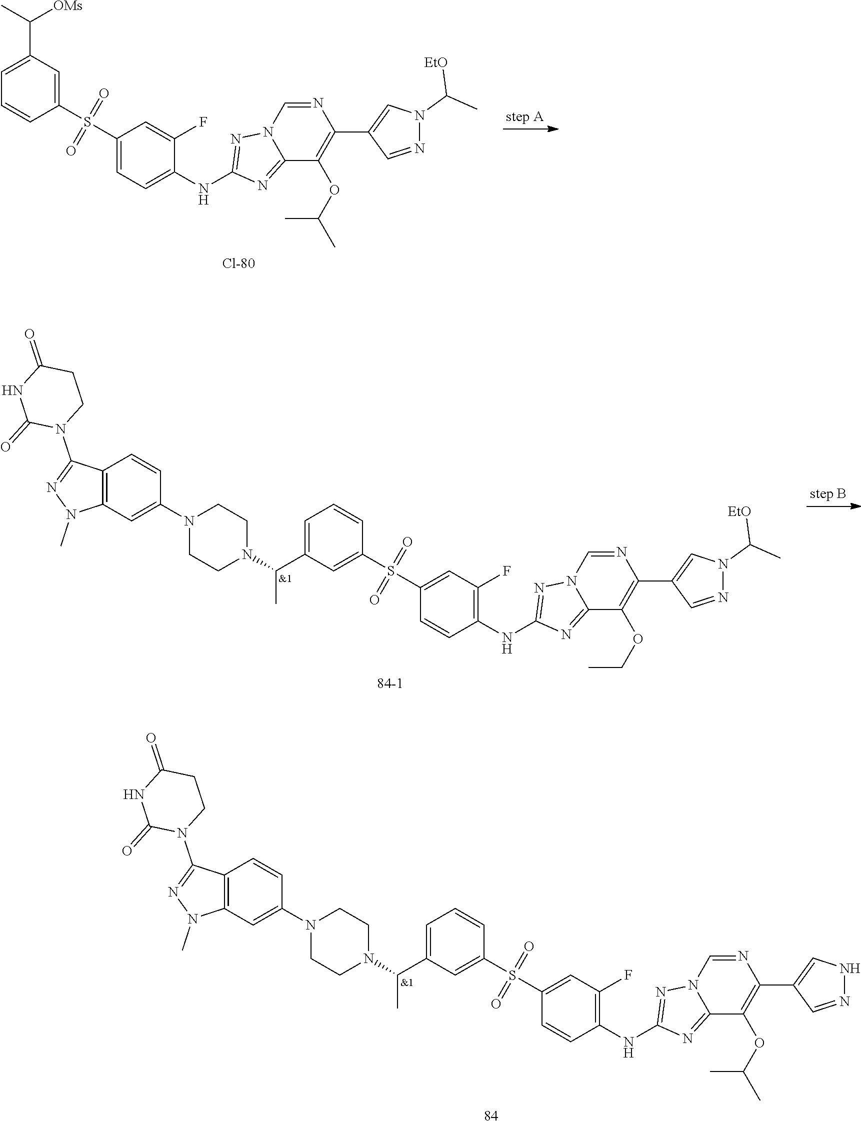

### Step A. 1-{6-[5-(1-{3-[4-({7-[1-(1-ethoxyethyl)pyrazol-4-yl]-8-isopropoxy-[1,2,4]triazolo[1,5-c]pyrimidin-2-yl}amino)-3-fluorobenzenesulfonyl]phenyl}ethyl)-2,5-diazabicyclo[4.1.0]heptan-2-yl]-1-methylindazol-3-yl}-1,3-diazinane-2,4-dione (84-1)

To a stirred mixture of 1-[1-methyl-6-(piperazin-1-yl)indazol-3-yl]-1,3-diazinane-2,4-dione hydrocholoride (CI-10, 47.75 mg, 0.145 mmol, 1 equiv) and potassium carbonate (120.57 mg, 0.870 mmol, 6 equiv) in acetonitrile (2 mL) was added 1-{3-[4-({7-[1-(1-ethoxyethyl)pyrazol-4-yl]-8-isopropoxy-[1,2,4]triazolo[1,5-c]pyrimidin-2-yl}amino)-3-fluorobenzenesulfonyl]phenyl}ethyl methanesulfonate (CI-80, 100 mg, 0.145 mmol, 1 equiv) and potassium iodide (24.14 mg, 0.145 mmol, 1 equiv). The resulting mixture was stirred for 1 h at 60° C. Desired product could be detected by LCMS. The resulting mixture was concentrated under reduced pressure. The residue was purified by silica gel column chromatography, eluted with methanol/dichloromethane (1:20) to afford 1-{6-[5-(1-{3-[4-({7-[1-(1-ethoxyethyl)pyrazol-4-yl]-8-isopropoxy-[1,2,4]triazolo[1,5-c]pyrimidin-2-yl}amino)-3-fluorobenzenesulfonyl]phenyl}ethyl)-2,5-diazabicyclo[4.1.0]heptan-2-yl]-1-methylindazol-3-yl}-1,3-diazinane-2,4-dione (84-1, 80 mg, 53.25%) as a white solid. LCMS (ES, m/z): [M+H]⁺ 906.4.

### Step B. 1-(6-{4-[(1S)-1-[3-(3-fluoro-4-{[8-isopropoxy-7-(1H-pyrazol-4-yl)-[1,2,4]triazolo[1,5-c]pyrimidin-2-yl]amino}benzenesulfonyl)phenyl]ethyl]piperazin-1-yl}-1-methylindazol-3-yl)-1,3-diazinane-2,4-dione (84)

To a stirred solution of 1-(6-{4-[(1S)-1-{3-[4-({7-[1-(1-ethoxyethyl)pyrazol-4-yl]-8-isopropoxy-[1,2,4]triazolo[1,5-c]pyrimidin-2-yl}amino)-3-fluorobenzenesulfonyl]phenyl}ethyl]piperazin-1-yl}-1-methylindazol-3-yl)-1,3-diazinane-2,4-dione (50 mg, 0.054 mmol, 1 equiv) in acetonitrile (5 mL) was added HCl (gas) in 1,4-dioxane (1 mL). The resulting mixture was stirred for 0.5 h at 0° C. Desired product could be detected by LCMS. The reaction was quenched with triethylamine at 0° C. The resulting mixture was concentrated under reduced pressure. The residue was purified by reversed-phase Combi-Flash, eluted with acetonitrile:water (0.5% ammonium bicarbonate)=30%-60%. This resulted in 1-(6-{4-[(1S)-1-[3-(3-fluoro-4-{[8-isopropoxy-7-(1H-pyrazol-4-yl)-[1,2,4]triazolo[1,5-c]pyrimidin-2-yl]amino}benzenesulfonyl)phenyl]ethyl]piperazin-1-yl}-1-methylindazol-3-yl)-1,3-diazinane-2,4-dione (84, 10 mg, 21.53%) as a white solid. LCMS (ES, m/z): [M+H]⁺ 848.3.

### Example 10. (R)-3-(5-((1r,3S)-3-(3-((3-fluoro-4-((8-isopropoxy-7-(1H-pyrazol-4-yl)-[1,2,4]triazolo[1,5-c]pyrimidin-2-yl)amino)phenyl)sulfonyl)benzyl)cyclobutyl)-1-oxoisoindolin-2-yl)piperidine-2,6-dione (79), (R)-3-(5-((1s,3R)-3-(3-((3-fluoro-4-((8-isopropoxy-7-(1H-pyrazol-4-yl)-[1,2,4]triazolo[1,5-c]pyrimidin-2-yl)amino)phenyl)sulfonyl)benzyl)cyclobutyl)-1-oxoisoindolin-2-yl)piperidine-2,6-dione (80), (S)-3-(5-((1r,3R)-3-(3-((3-fluoro-4-((8-isopropoxy-7-(1H-pyrazol-4-yl)-[1,2,4]triazolo[1,5-c]pyrimidin-2-yl)amino)phenyl)sulfonyl)benzyl)cyclobutyl)-1-oxoisoindolin-2-yl)piperidine-2,6-dione (81), (S)-3-(5-((1s,3S)-3-(3-((3-fluoro-4-((8-isopropoxy-7-(1H-pyrazol-4-yl)-[1,2,4]triazolo[1,5-c]pyrimidin-2-yl)amino)phenyl)sulfonyl)benzyl)cyclobutyl)-1-oxoisoindolin-2-yl)piperidine-2,6-dione (82)

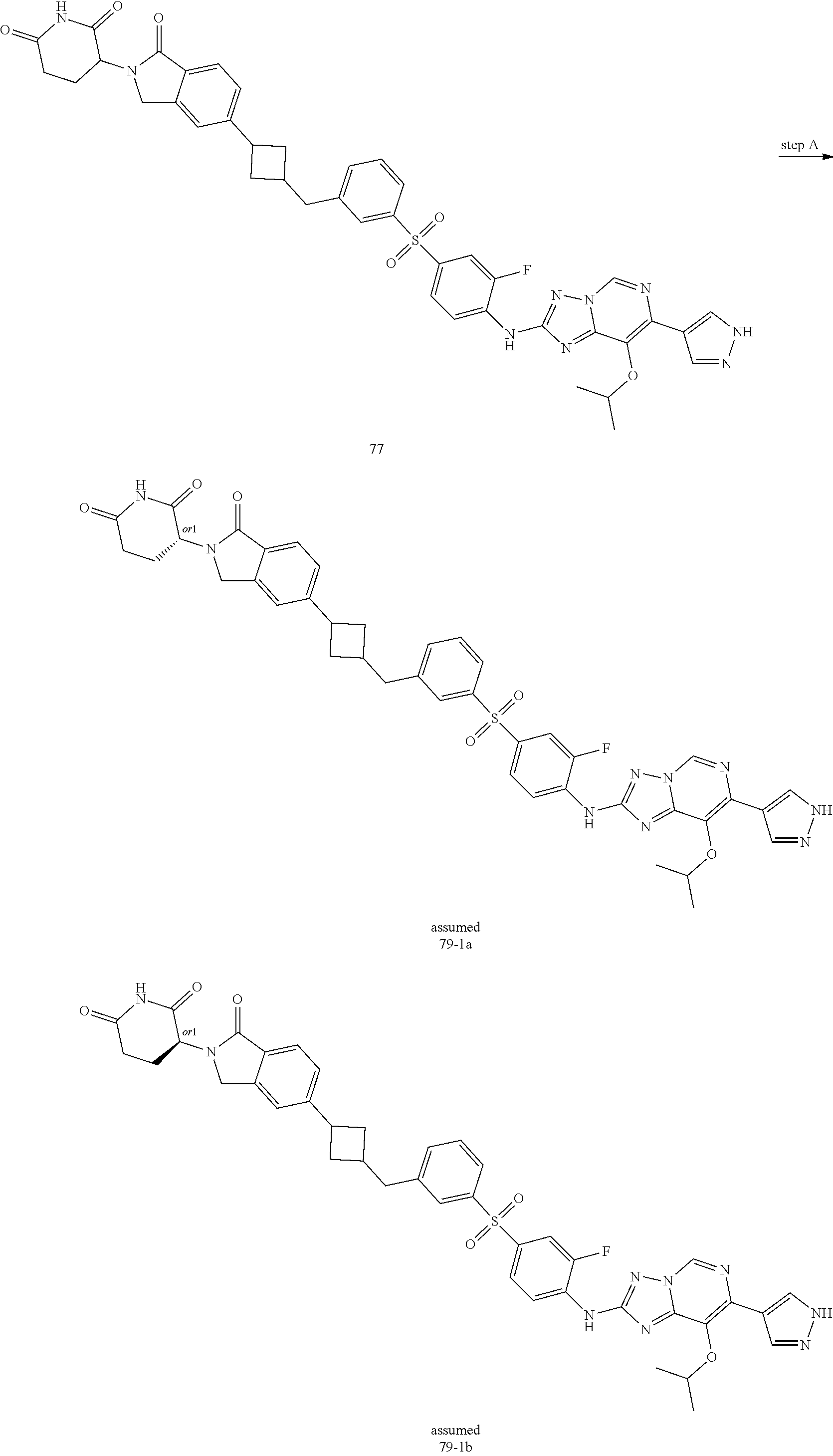 [SMILES: CC(C)Oc1c(-c2cn[nH]c2)ncn2nc(Nc3ccc(S(=O)(=O)c4cccc(CC5CC(c6ccc7c(c6)CN(C6CCC(=O)NC6=O)C7=O)C5)c4)cc3F)nc12 ; CC(C)Oc1c(-c2cn[nH]c2)ncn2nc(Nc3ccc(S(=O)(=O)c4cccc(CC5CC(c6ccc7c(c6)CN([C@@H]6CCC(=O)NC6=O)C7=O)C5)c4)cc3F)nc12 ; CC(C)Oc1c(-c2cn[nH]c2)ncn2nc(Nc3ccc(S(=O)(=O)c4cccc(CC5CC(c6ccc7c(c6)CN([C@H]6CCC(=O)NC6=O)C7=O)C5)c4)cc3F)nc12]

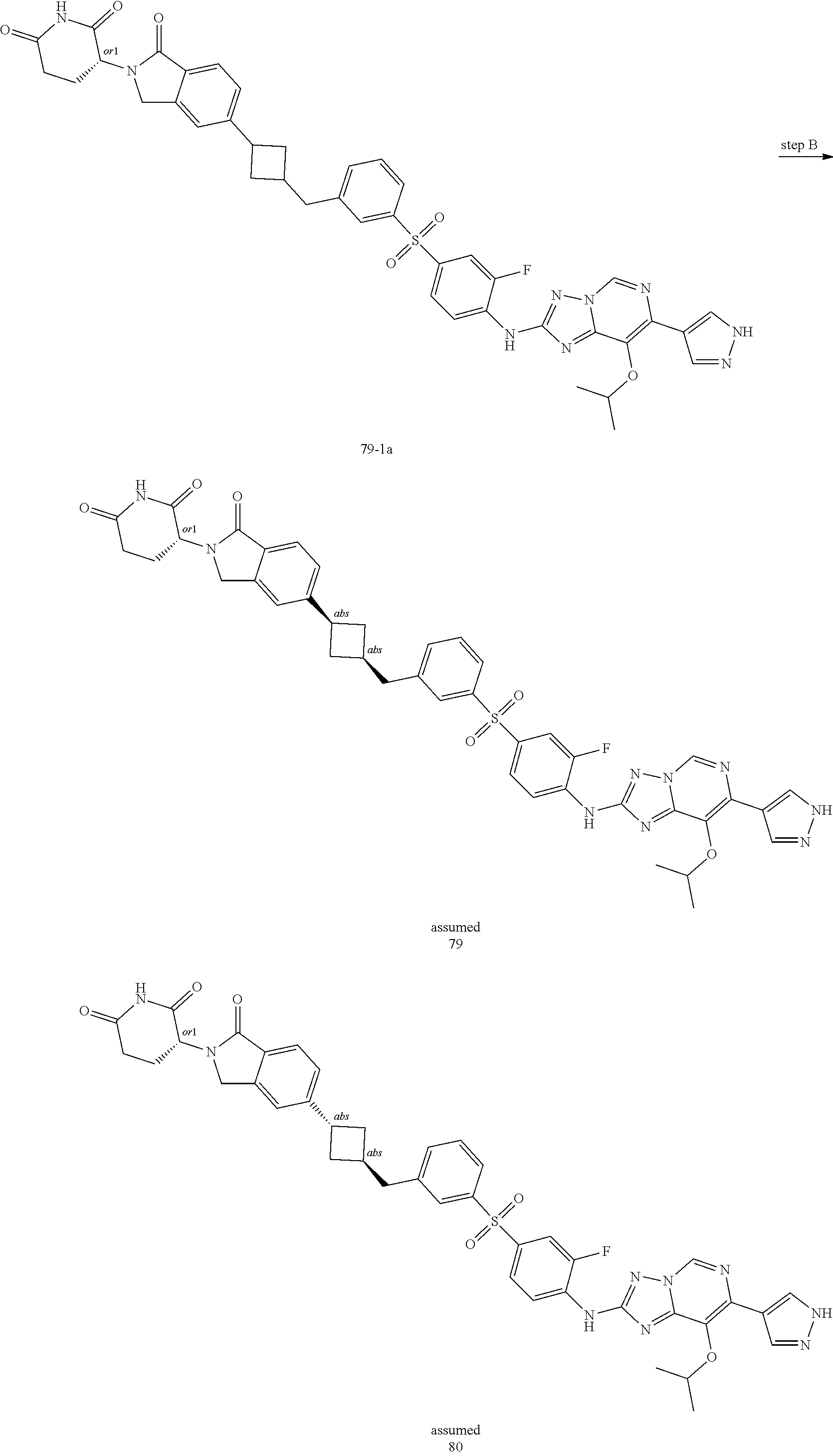 [SMILES: CC(C)Oc1c(-c2cn[nH]c2)ncn2nc(Nc3ccc(S(=O)(=O)c4cccc(CC5CC(c6ccc7c(c6)CN([C@@H]6CCC(=O)NC6=O)C7=O)C5)c4)cc3F)nc12 ; CC(C)Oc1c(-c2cn[nH]c2)ncn2nc(Nc3ccc(S(=O)(=O)c4cccc(C[C@H]5C[C@@H](c6ccc7c(c6)CN([C@@H]6CCC(=O)NC6=O)C7=O)C5)c4)cc3F)nc12 ; CC(C)Oc1c(-c2cn[nH]c2)ncn2nc(Nc3ccc(S(=O)(=O)c4cccc(C[C@H]5C[C@H](c6ccc7c(c6)CN([C@@H]6CCC(=O)NC6=O)C7=O)C5)c4)cc3F)nc12]

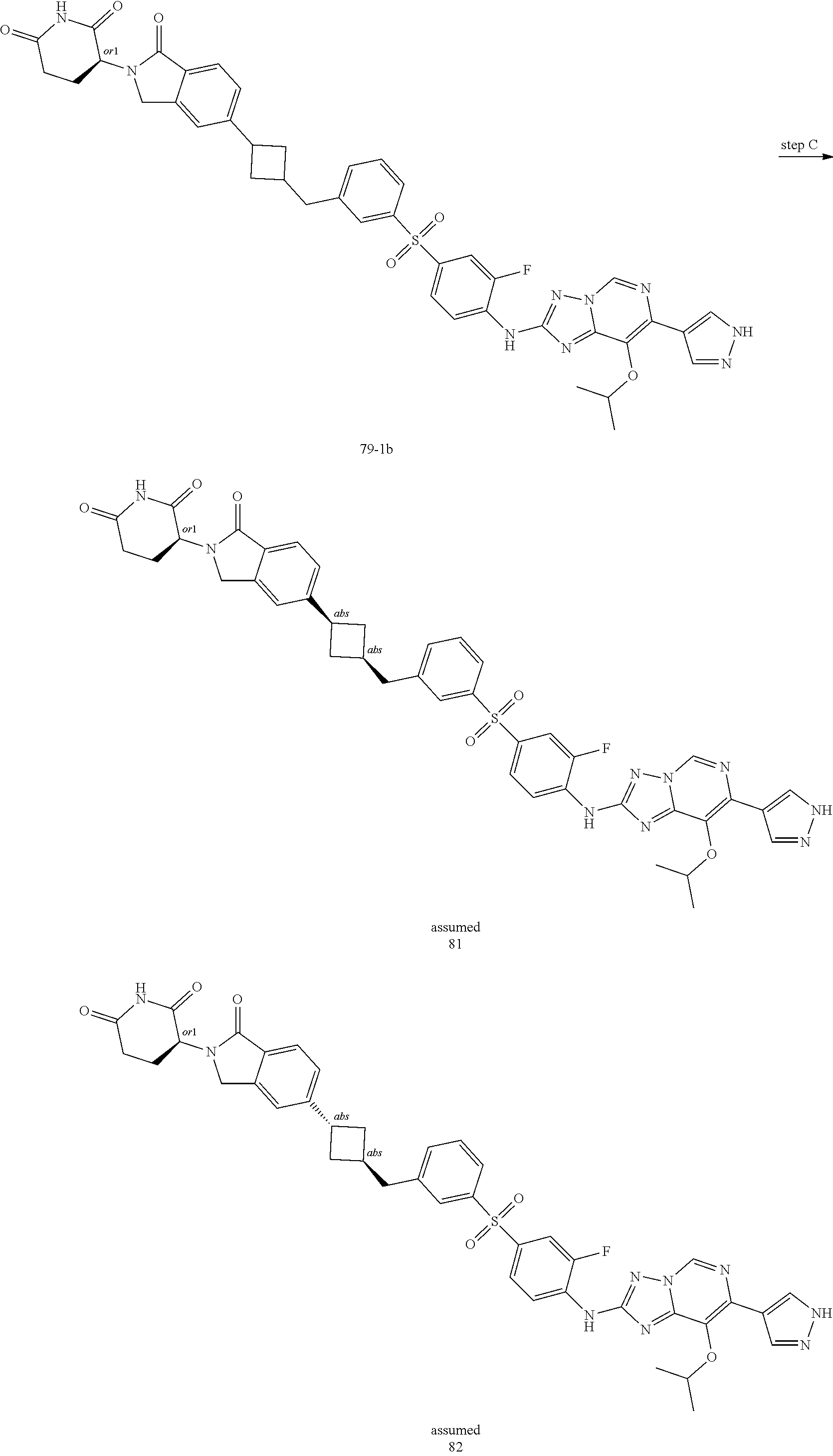 [SMILES: CC(C)Oc1c(-c2cn[nH]c2)ncn2nc(Nc3ccc(S(=O)(=O)c4cccc(CC5CC(c6ccc7c(c6)CN([C@H]6CCC(=O)NC6=O)C7=O)C5)c4)cc3F)nc12 ; CC(C)Oc1c(-c2cn[nH]c2)ncn2nc(Nc3ccc(S(=O)(=O)c4cccc(C[C@H]5C[C@@H](c6ccc7c(c6)CN([C@H]6CCC(=O)NC6=O)C7=O)C5)c4)cc3F)nc12 ; CC(C)Oc1c(-c2cn[nH]c2)ncn2nc(Nc3ccc(S(=O)(=O)c4cccc(C[C@H]5C[C@H](c6ccc7c(c6)CN([C@H]6CCC(=O)NC6=O)C7=O)C5)c4)cc3F)nc12]

### Step A. (R)-3-(5-(3-(3-((3-fluoro-4-((8-isopropoxy-7-(1H-pyrazol-4-yl)-[1,2,4]triazolo[1,5-c]pyrimidin-2-yl)amino)phenyl)sulfonyl)benzyl)cyclobutyl)-1-oxoisoindolin-2-yl)piperidine-2,6-dione (79-1a) and (S)-3-(5-(3-(3-((3-fluoro-4-((8-isopropoxy-7-(1H-pyrazol-4-yl)-[1,2,4]triazolo[1,5-c]pyrimidin-2-yl)amino)phenyl)sulfonyl)benzyl)cyclobutyl)-1-oxoisoindolin-2-yl)piperidine-2,6-dione (79-1b)

The 3-(5-(3-(3-((3-fluoro-4-((8-isopropoxy-7-(1H-pyrazol-4-yl)-[1,2,4]triazolo[1,5-c]pyrimidin-2-yl)amino)phenyl)sulfonyl)benzyl)cyclobutyl)-1-oxoisoindolin-2-yl)piperidine-2,6-dione (77, 100 mg) was purified by prep chiral HPLC with the following conditions: column, CHIRALPAK IC-3; Mobile Phase:methyl tert-butyl ether (0.1% DEA):methanol=50:50; flow Rate:1.67 ml/min, temperature:ambient. The prepeak fractions were evaporated to dryness to afford (R)-3-(5-(3-(3-((3-fluoro-4-((8-isopropoxy-7-(1H-pyrazol-4-yl)-[1,2,4]triazolo[1,5-c]pyrimidin-2-yl)amino)phenyl)sulfonyl)benzyl)cyclobutyl)-1-oxoisoindolin-2-yl)piperidine-2,6-dione (79-1a, 46 mg, 46%) as a white solid. LCMS (ES, m/z): [M+H]⁺ 804.35. The postpeak fractions were evaporated to dryness to afford (S)-3-(5-(3-(3-((3-fluoro-4-((8-isopropoxy-7-(1H-pyrazol-4-yl)-[1,2,4]triazolo[1,5-c]pyrimidin-2-yl)amino)phenyl)sulfonyl)benzyl)cyclobutyl)-1-oxoisoindolin-2-yl)piperidine-2,6-dione (79-1b, 44 mg, 44%) as a white solid. LCMS (ES, m/z): [M+H]⁺ 804.35.

### Step B. rel-(R)-3-(5-((1r,3S)-3-(3-((3-fluoro-4-((8-isopropoxy-7-(1H-pyrazol-4-yl)-[1,2,4]triazolo[1,5-c]pyrimidin-2-yl)amino)phenyl)sulfonyl)benzyl)cyclobutyl)-1-oxoisoindolin-2-yl)piperidine-2,6-dione (79) and rel-(R)-3-(5-((1s,3R)-3-(3-((3-fluoro-4-((8-isopropoxy-7-(1H-pyrazol-4-yl)-[1,2,4]triazolo[1,5-c]pyrimidin-2-yl)amino)phenyl)sulfonyl)benzyl)cyclobutyl)-1-oxoisoindolin-2-yl)piperidine-2,6-dione (80)

The (R)-3-(5-(3-(3-((3-fluoro-4-((8-isopropoxy-7-(1H-pyrazol-4-yl)-[1,2,4]triazolo[1,5-c]pyrimidin-2-yl)amino)phenyl)sulfonyl)benzyl)cyclobutyl)-1-oxoisoindolin-2-yl)piperidine-2,6-dione (79-1a, 46 mg) was purified by prep chiral HPLC with the following conditions: column, CHIRAL Cellulose-SB; mobile phase: (MtBE:Hex=1:1)(0.1% trifluoroacetic acid):methanol=85:15; detector, UV 254 nm; Flow rate:1.67 ml/min, temperature:ambient. The prepeak fractions were evaporated to dryness to afford rel-(R)-3-(5-((1r,3S)-3-(3-((3-fluoro-4-((8-isopropoxy-7-(1H-pyrazol-4-yl)-[1,2,4]triazolo[1,5-c]pyrimidin-2-yl)amino)phenyl)sulfonyl)benzyl)cyclobutyl)-1-oxoisoindolin-2-yl)piperidine-2,6-dione (79, 10 mg, 24.82%) as a white solid. LCMS (ES, m/z): [M+H]⁺ 804.35. The postpeak fractions were evaporated to dryness to afford rel-(R)-3-(5-((1s,3R)-3-(3-((3-fluoro-4-((8-isopropoxy-7-(1H-pyrazol-4-yl)-[1,2,4]triazolo[1,5-c]pyrimidin-2-yl)amino)phenyl)sulfonyl)benzyl)cyclobutyl)-1-oxoisoindolin-2-yl)piperidine-2,6-dione (80, 12 mg, 29.76%) as a white solid. LCMS (ES, m/z): [M+H]⁺ 804.35.

### Step C. rel-(S)-3-(5-((1r,3R)-3-(3-((3-fluoro-4-((8-isopropoxy-7-(1H-pyrazol-4-yl)-[1,2,4]triazolo[1,5-c]pyrimidin-2-yl)amino)phenyl)sulfonyl)benzyl)cyclobutyl)-1-oxoisoindolin-2-yl)piperidine-2,6-dione (81) and rel-(S)-3-(5-((1s,3S)-3-(3-((3-fluoro-4-((8-isopropoxy-7-(1H-pyrazol-4-yl)-[1,2,4]triazolo[1,5-c]pyrimidin-2-yl)amino)phenyl)sulfonyl)benzyl)cyclobutyl)-1-oxoisoindolin-2-yl)piperidine-2,6-dione (82)

The (S)-3-(5-(3-(3-((3-fluoro-4-((8-isopropoxy-7-(1H-pyrazol-4-yl)-[1,2,4]triazolo[1,5-c]pyrimidin-2-yl)amino)phenyl)sulfonyl)benzyl)cyclobutyl)-1-oxoisoindolin-2-yl)piperidine-2,6-dione (79-lb, 44 mg) was purified by prep chiral HPLC with the following conditions: column, CHIRAL Cellulose-SB; mobile phase:(methyl tert-butyl ether:Hex=1:1)(0.1% trifluoroacetic acid):methanol=85:15; detector, UV 254 nm; flow rate:1.67 ml/min. temperature:ambient. The prepeak fractions were evaporated to dryness to afford rel-(S)-3-(5-((1r,3R)-3-(3-((3-fluoro-4-((8-isopropoxy-7-(1H-pyrazol-4-yl)-[1,2,4]triazolo[1,5-c]pyrimidin-2-yl)amino)phenyl)sulfonyl)benzyl)cyclobutyl)-1-oxoisoindolin-2-yl)piperidine-2,6-dione (81, 16 mg, 39.72%) as a white solid. LCMS (ES, m/z): [M+H]⁺ 804.35. The postpeak fractions were evaporated to dryness to afford rel-(S)-3-(5-((1s,3S)-3-(3-((3-fluoro-4-((8-isopropoxy-7-(1H-pyrazol-4-yl)-[1,2,4]triazolo[1,5-c]pyrimidin-2-yl)amino)phenyl) sulfonyl)benzyl)cyclobutyl)-1-oxoisoindolin-2-yl)piperidine-2,6-dione (82, 12 mg, 29.79%) as a white solid. LCMS (ES, m/z): [M+H]⁺ 804.35.

### Example 11. rel-1-(6-((1s,3r)-3-(3-((3-fluoro-4-((8-isopropoxy-7-(1H-pyrazol-4-yl)-[1,2,4]triazolo[1,5-c]pyrimidin-2-yl)amino)phenyl)sulfonyl)benzyl)cyclobutyl)-1-methyl-1H-indazol-3-yl)dihydropyrimidine-2,4(1H,3H)-dione (89) and rel-1-(6-((1r,3s)-3-(3-((3-fluoro-4-((8-isopropoxy-7-(1H-pyrazol-4-yl)-[1,2,4]triazolo[1,5-c]pyrimidin-2-yl)amino)phenyl)sulfonyl)benzyl)cyclobutyl)-1-methyl-1H-indazol-3-yl)dihydropyrimidine-2,4(1H,3H)-dione (90)

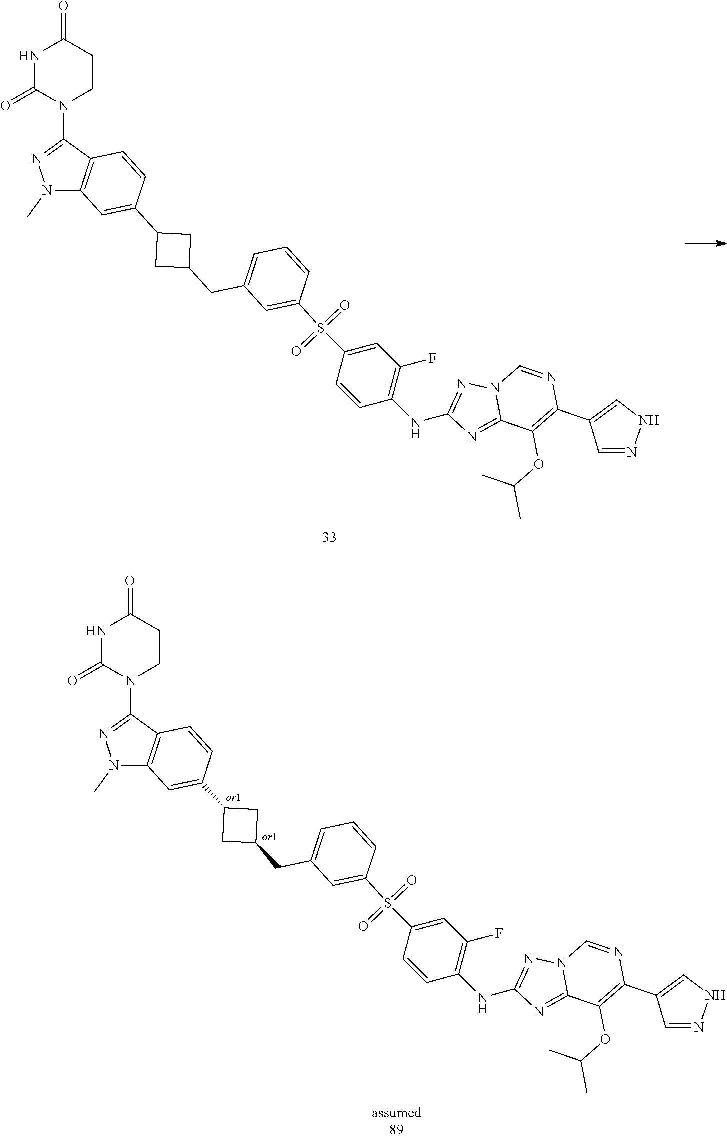 [SMILES: CC(C)Oc1c(-c2cn[nH]c2)ncn2nc(Nc3ccc(S(=O)(=O)c4cccc(CC5CC(c6ccc7c(N8CCC(=O)NC8=O)nn(C)c7c6)C5)c4)cc3F)nc12 ; CC(C)Oc1c(-c2cn[nH]c2)ncn2nc(Nc3ccc(S(=O)(=O)c4cccc(C[C@H]5C[C@H](c6ccc7c(N8CCC(=O)NC8=O)nn(C)c7c6)C5)c4)cc3F)nc12]

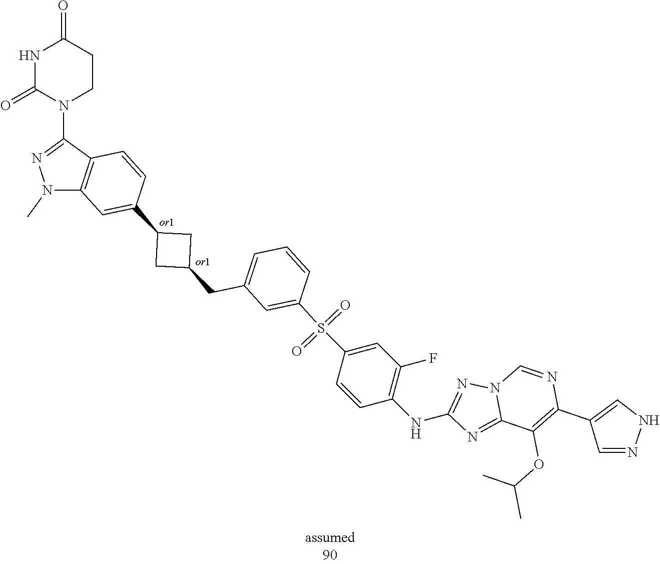 [SMILES: CC(C)Oc1c(-c2cn[nH]c2)ncn2nc(Nc3ccc(S(=O)(=O)c4cccc(C[C@H]5C[C@@H](c6ccc7c(N8CCC(=O)NC8=O)nn(C)c7c6)C5)c4)cc3F)nc12]

The 1-(6-(3-(3-((3-fluoro-4-((8-isopropoxy-7-(1H-pyrazol-4-yl)-[1,2,4]triazolo[1,5-c]pyrimidin-2-yl)amino)phenyl)sulfonyl)benzyl)cyclobutyl)-1-methyl-1H-indazol-3-yl)dihydropyrimidine-2,4(1H,3H)-dione (33, 30 mg) was separation by Chiral HPLC, separation method: Column: CHIRAL ART Cellulose-SB, 3*25 cm, 5 μm; Mobile Phase A: Hex: MtBE=1:1(0.5% 2M NH3-MEOH), Mobile Phase B: ethanol; Flow rate: 40 mL/min; Gradient: isocratic 20; Wave Length: 306/254 nm; RT1(min): 46.10; RT2(min): 59.63; Sample Solvent: dimethyl sulfoxide; Injection Volume: 0.3 mL; Number Of Runs: 4. The prepeak fractions were evaporated to dryness to afford rel-1-(6-((1s,3r)-3-(3-((3-fluoro-4-((8-isopropoxy-7-(1H-pyrazol-4-yl)-[1,2,4]triazolo[1,5-c]pyrimidin-2-yl)amino)phenyl)sulfonyl)benzyl)cyclobutyl)-1-methyl-1H-indazol-3-yl)dihydropyrimidine-2,4(1H,3H)-dione (89, 10.4 mg, 34.11%) as an off-white solid. LCMS (ESI, m/z): [M+H]⁺ 804.2. The postpeak fractions were evaporated to dryness to afford rel-1-(6-((1r,3s)-3-(3-((3-fluoro-4-((8-isopropoxy-7-(1H-pyrazol-4-yl)-[1,2,4]triazolo[1,5-c]pyrimidin-2-yl)amino)phenyl)sulfonyl)benzyl)cyclobutyl)-1-methyl-1H-indazol-3-yl)dihydropyrimidine-2,4(1H,3H)-dione (90, 12.8 mg, 42.67%) as an off-white solid. LCMS (ESI, m/z): [M+H]⁺ 804.1.

### Example 12. rel-(R)-1-(6-(1-((1-((3-fluoro-4-((8-isopropoxy-7-(1H-pyrazol-4-yl)-[1,2,4]triazolo[1,5-c]pyrimidin-2-yl)amino)phenyl)sulfonyl)piperidin-3-yl)methyl)piperidin-4-yl)-1-methyl-1H-indazol-3-yl)dihydropyrimidine-2,4(1H,3H)-dione (94) and rel-(R)-1-(6-(1-((1-((3-fluoro-4-((8-isopropoxy-7-(1H-pyrazol-4-yl)-[1,2,4]triazolo[1,5-c]pyrimidin-2-yl)amino)phenyl)sulfonyl)piperidin-3-yl)methyl) piperidin-4-yl)-1-methyl-1H-indazol-3-yl)dihydropyrimidine-2,4(1H,3H)-dione (95)

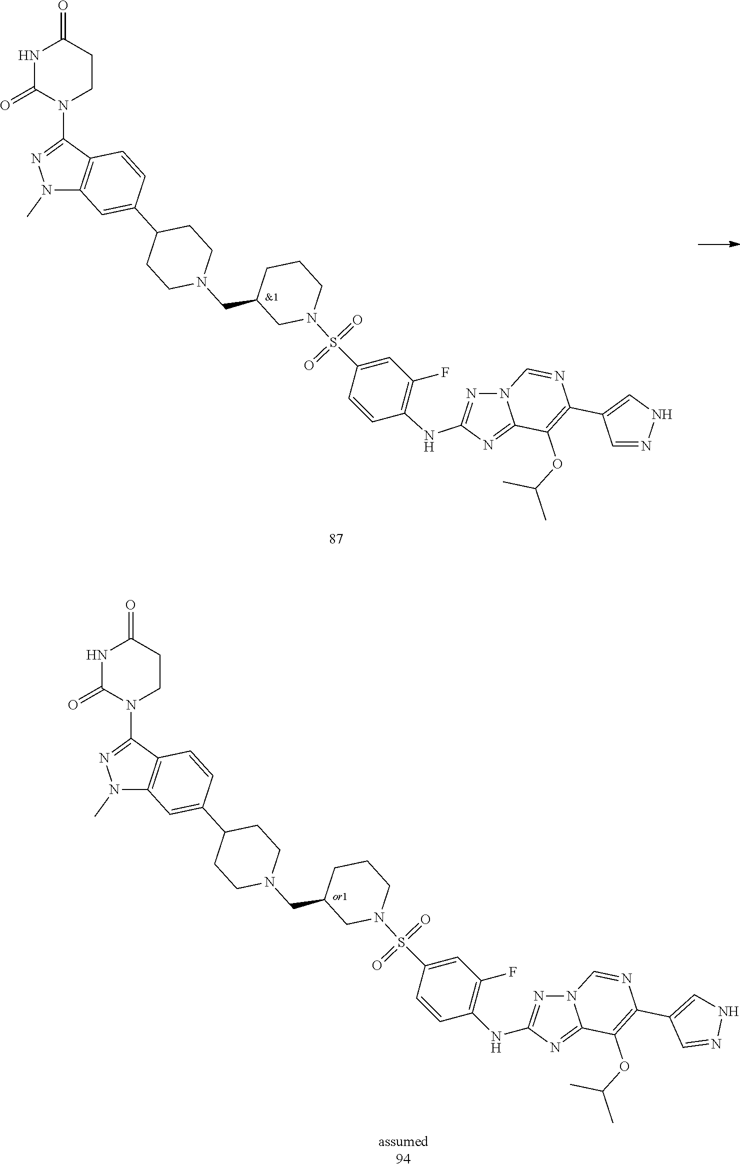 [SMILES: CC(C)Oc1c(-c2cn[nH]c2)ncn2nc(Nc3ccc(S(=O)(=O)N4CCC[C@H](CN5CCC(c6ccc7c(N8CCC(=O)NC8=O)nn(C)c7c6)CC5)C4)cc3F)nc12 ; CC(C)Oc1c(-c2cn[nH]c2)ncn2nc(Nc3ccc(S(=O)(=O)N4CCC[C@H](CN5CCC(c6ccc7c(N8CCC(=O)NC8=O)nn(C)c7c6)CC5)C4)cc3F)nc12]

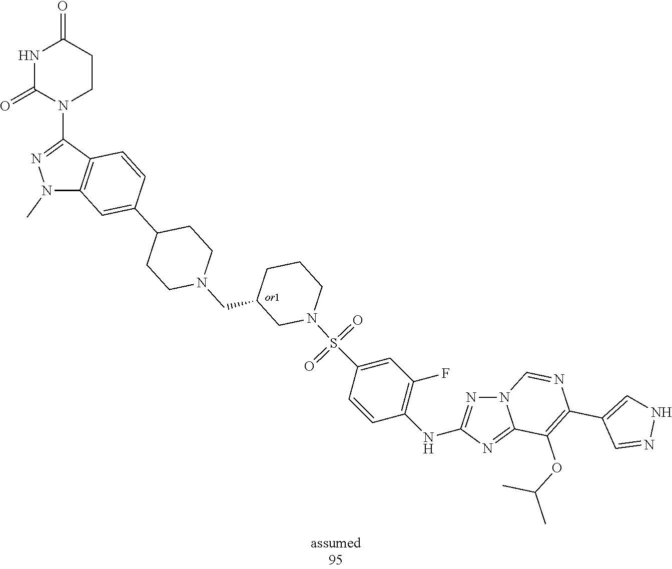 [SMILES: CC(C)Oc1c(-c2cn[nH]c2)ncn2nc(Nc3ccc(S(=O)(=O)N4CCC[C@@H](CN5CCC(c6ccc7c(N8CCC(=O)NC8=O)nn(C)c7c6)CC5)C4)cc3F)nc12]

The rac-(R)-1-(6-(1-((1-((3-fluoro-4-((8-isopropoxy-7-(1H-pyrazol-4-yl)-[1, 2, 4]triazolo[1,5-c]pyrimidin-2-yl)amino)phenyl)sulfonyl)piperidin-3-yl)methyl)piperidin-4-yl)-1-methyl-1H-indazol-3-yl)dihydropyrimidine-2,4(1H,3H)-dione (87, 60 mg) was separated by prep chiral HPLC with the following conditions (Column: CHIRALPAK IG, 3*25 cm, 5 μm; Mobile Phase A: methyl tert-butyl ether (10 mM NH₃-methanol), Mobile Phase B: methanol; Flow rate: 40 mL/min; Gradient: isocratic 30; Wave Length: 214/289 nm; RT1(min): 14.8; RT2(min): 21.6; Sample Solvent: methanol: dichloromethane=8:1; Injection Volume: 0.8 mL; Number Of Runs: 3). The prepeak fractions were evaporated to dryness to afford rel-(R)-1-(6-(1-((1-((3-fluoro-4-((8-isopropoxy-7-(1H-pyrazol-4-yl)-[1,2,4]triazolo[1,5-c]pyrimidin-2-yl)amino)phenyl)sulfonyl)piperidin-3-yl)methyl)piperidin-4-yl)-1-methyl-1H-indazol-3-yl)dihydropyrimidine-2,4(1H,3H)-dione (94, 9.3 mg, 30.81%) as a white solid. LCMS (ESI, m/z): [M+H]⁺ 839.9. The postpeak fractions were evaporated to dryness to afford rel-(R)-1-(6-(1-((1-((3-fluoro-4-((8-isopropoxy-7-(1H-pyrazol-4-yl)-[1,2,4]triazolo[1,5-c]pyrimidin-2-yl)amino)phenyl)sulfonyl)piperidin-3-yl)methyl) piperidin-4-yl)-1-methyl-1H-indazol-3-yl)dihydropyrimidine-2,4(1H,3H)-dione (95, 10 mg, 33.17%) as a white solid. LCMS (ESI, m/z): [M+H]⁺ 839.9.

### Example 13. rel-1-(6-((R)-1-(((R)-1-((3-fluoro-4-((8-isopropoxy-7-(1H-pyrazol-4-yl)-[1,2,4]triazolo[1,5-c]pyrimidin-2-yl)amino)phenyl)sulfonyl)piperidin-3-yl)methyl)-2,2-dimethylpiperidin-4-yl)-1-methyl-1H-indazol-3-yl)dihydropyrimidine-2,4(1H,3H)-dione (96) and rel-1-(6-((R)-1-(((S)-1-((3-fluoro-4-((8-isopropoxy-7-(1H-pyrazol-4-yl)-[1,2,4]triazolo[1,5-c]pyrimidin-2-yl)amino)phenyl) sulfonyl)piperidin-3-yl)methyl)-2,2-dimethylpiperidin-4-yl)-1-methyl-1H-indazol-3-yl)dihydropyrimidine-2,4(1H,3H)-dione (97), rel-1-(6-((R)-1-(((R)-1-((3-fluoro-4-((8-isopropoxy-7-(1H-pyrazol-4-yl)-[1,2,4]triazolo[1,5-c]pyrimidin-2-yl)amino)phenyl)sulfonyl)piperidin-3-yl)methyl)-2,2-dimethylpiperidin-4-yl)-1-methyl-1H-indazol-3-yl)dihydropyrimidine-2,4(1H,3H)-dione (102) and rel-1-(6-((R)-1-(((S)-1-((3-fluoro-4-((8-isopropoxy-7-(1H-pyrazol-4-yl)-[1,2,4]triazolo[1,5-c]pyrimidin-2-yl)amino)phenyl)sulfonyl)piperidin-3-yl)methyl)-2,2-dimethylpiperidin-4-yl)-1-methyl-1H-indazol-3-yl)dihydropyrimidine-2,4(1H,3H)-dione (103), rel-1-(6-((R)-1-(((S)-1-((3-fluoro-4-((8-isopropoxy-7-(1H-pyrazol-4-yl)-[1,2,4]triazolo[1,5-c]pyrimidin-2-yl)amino)phenyl)sulfonyl)piperidin-3-yl)methyl)-2,2-dimethylpiperidin-4-yl)-1-methyl-1H-indazol-3-yl)dihydropyrimidine-2,4(1H,3H)-dione (104) and rel-1-(6-((R)-1-(((R)-1-((3-fluoro-4-((8-isopropoxy-7-(1H-pyrazol-4-yl)-[1,2,4]triazolo[1,5-c]pyrimidin-2-yl)amino)phenyl)sulfonyl)piperidin-3-yl)methyl)-2,2-dimethylpiperidin-4-yl)-1-methyl-1H-indazol-3-yl)dihydropyrimidine-2,4(1H,3H)-dione (105)

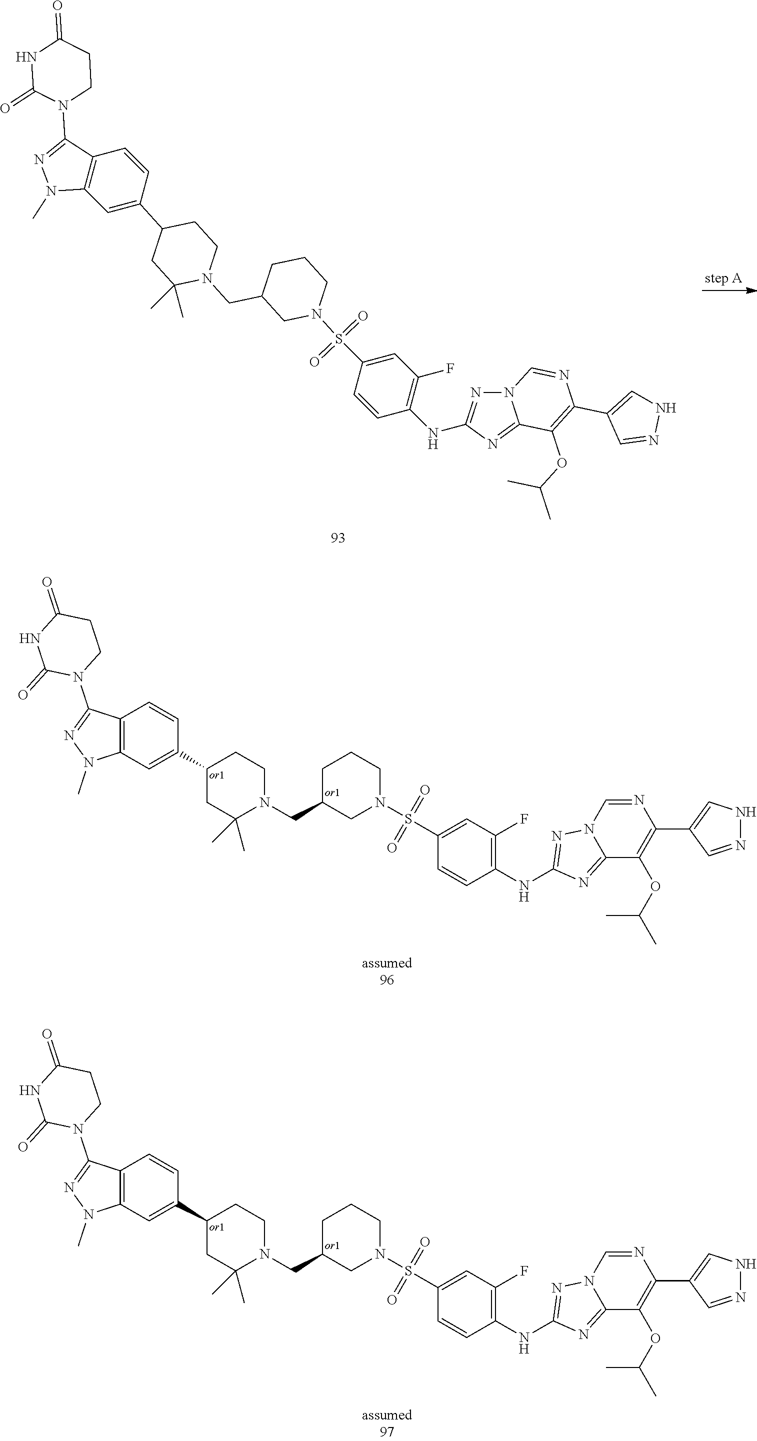 [SMILES: CC(C)Oc1c(-c2cn[nH]c2)ncn2nc(Nc3ccc(S(=O)(=O)N4CCCC(CN5CCC(c6ccc7c(N8CCC(=O)NC8=O)nn(C)c7c6)CC5(C)C)C4)cc3F)nc12 ; CC(C)Oc1c(-c2cn[nH]c2)ncn2nc(Nc3ccc(S(=O)(=O)N4CCC[C@H](CN5CC[C@@H](c6ccc7c(N8CCC(=O)NC8=O)nn(C)c7c6)CC5(C)C)C4)cc3F)nc12 ; CC(C)Oc1c(-c2cn[nH]c2)ncn2nc(Nc3ccc(S(=O)(=O)N4CCC[C@H](CN5CC[C@H](c6ccc7c(N8CCC(=O)NC8=O)nn(C)c7c6)CC5(C)C)C4)cc3F)nc12]

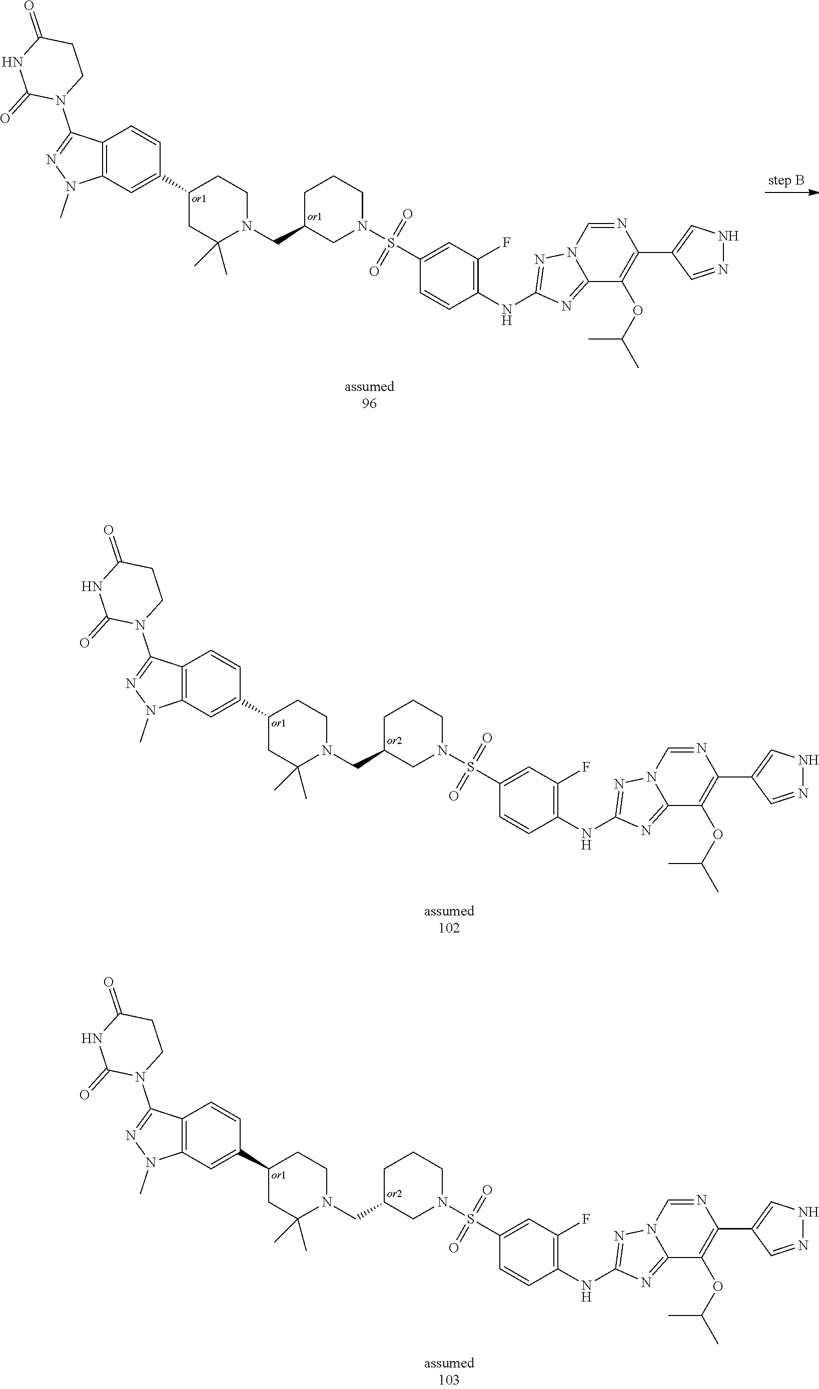 [SMILES: CC(C)Oc1c(-c2cn[nH]c2)ncn2nc(Nc3ccc(S(=O)(=O)N4CCC[C@H](CN5CC[C@@H](c6ccc7c(N8CCC(=O)NC8=O)nn(C)c7c6)CC5(C)C)C4)cc3F)nc12 ; CC(C)Oc1c(-c2cn[nH]c2)ncn2nc(Nc3ccc(S(=O)(=O)N4CCC[C@H](CN5CC[C@@H](c6ccc7c(N8CCC(=O)NC8=O)nn(C)c7c6)CC5(C)C)C4)cc3F)nc12 ; CC(C)Oc1c(-c2cn[nH]c2)ncn2nc(Nc3ccc(S(=O)(=O)N4CCC[C@@H](CN5CC[C@H](c6ccc7c(N8CCC(=O)NC8=O)nn(C)c7c6)CC5(C)C)C4)cc3F)nc12]

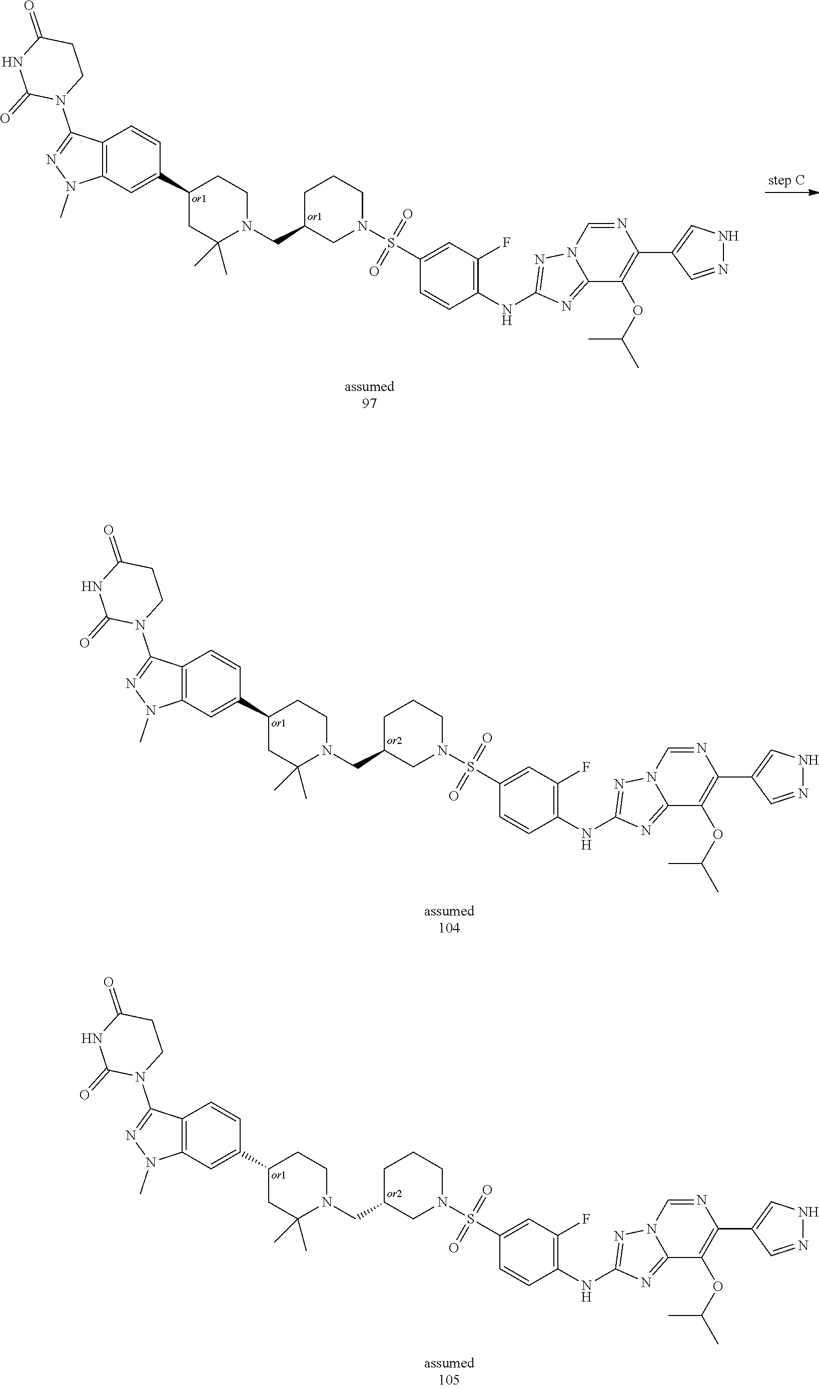 [SMILES: CC(C)Oc1c(-c2cn[nH]c2)ncn2nc(Nc3ccc(S(=O)(=O)N4CCC[C@H](CN5CC[C@H](c6ccc7c(N8CCC(=O)NC8=O)nn(C)c7c6)CC5(C)C)C4)cc3F)nc12 ; CC(C)Oc1c(-c2cn[nH]c2)ncn2nc(Nc3ccc(S(=O)(=O)N4CCC[C@H](CN5CC[C@H](c6ccc7c(N8CCC(=O)NC8=O)nn(C)c7c6)CC5(C)C)C4)cc3F)nc12 ; CC(C)Oc1c(-c2cn[nH]c2)ncn2nc(Nc3ccc(S(=O)(=O)N4CCC[C@@H](CN5CC[C@@H](c6ccc7c(N8CCC(=O)NC8=O)nn(C)c7c6)CC5(C)C)C4)cc3F)nc12]

### Step A. rel-1-(6-((R)-1-(((R)-1-((3-fluoro-4-((8-isopropoxy-7-(1H-pyrazol-4-yl)-[1,2,4]triazolo[1,5-c]pyrimidin-2-yl)amino)phenyl)sulfonyl)piperidin-3-yl)methyl)-2,2-dimethylpiperidin-4-yl)-1-methyl-1H-indazol-3-yl)dihydropyrimidine-2,4(1H,3H)-dione (96) and rel-1-(6-((R)-1-(((S)-1-((3-fluoro-4-((8-isopropoxy-7-(1H-pyrazol-4-yl)-[1,2,4]triazolo[1,5-c]pyrimidin-2-yl)amino)phenyl)sulfonyl)piperidin-3-yl)methyl)-2,2-dimethylpiperidin-4-yl)-1-methyl-1H-indazol-3-yl)dihydropyrimidine-2,4(1H,3H)-dione (97)

1-(6-(1-((1-((3-fluoro-4-((8-isopropoxy-7-(1H-pyrazol-4-yl)-[1,2,4]triazolo[1,5-c]pyrimidin-2-yl)amino)phenyl)sulfonyl)piperidin-3-yl)methyl)-2,2-dimethylpiperidin-4-yl)-1-methyl-1H-indazol-3-yl)dihydropyrimidine-2,4(1H,3H)-dione (93, 60 mg) was separated by reverse flash chromatography with the following conditions: column, C18 silica gel; mobile phase, acetonitrile in water, 5% to 50% gradient in 30 min; detector, UV 254 nm. The prepeak fractions were evaporated to dryness to afford rel-1-(6-((R)-1-(((R)-1-((3-fluoro-4-((8-isopropoxy-7-(1H-pyrazol-4-yl)-[1,2,4]triazolo[1,5-c]pyrimidin-2-yl)amino)phenyl) sulfonyl)piperidin-3-yl)methyl)-2,2-dimethylpiperidin-4-yl)-1-methyl-1H-indazol-3-yl)dihydropyrimidine-2,4(1H,3H)-dione (96, 9.5 mg, 9.70%) as a white solid. LCMS (ESI, m/z): [M+H]⁺ 868.3. The postpeak fractions were evaporated to dryness to afford rel-1-(6-((R)-1-(((S)-1-((3-fluoro-4-((8-isopropoxy-7-(1H-pyrazol-4-yl)-[1,2,4]triazolo[1,5-c]pyrimidin-2-yl)amino)phenyl)sulfonyl)piperidin-3-yl)methyl)-2,2-dimethylpiperidin-4-yl)-1-methyl-1H-indazol-3-yl)dihydropyrimidine-2,4(1H,3H)-dione (97, 9.5 mg, 9.70%) as a white solid. LCMS (ESI, m/z): [M+H]⁺ 868.3.

### Step B. rel-1-(6-((R)-1-(((R)-1-((3-fluoro-4-((8-isopropoxy-7-(1H-pyrazol-4-yl)-[1,2,4]triazolo[1,5-c]pyrimidin-2-yl)amino)phenyl)sulfonyl)piperidin-3-yl)methyl)-2,2-dimethylpiperidin-4-yl)-1-methyl-1H-indazol-3-yl)dihydropyrimidine-2,4(1H,3H)-dione (102) and rel-1-(6-((R)-1-(((S)-1-((3-fluoro-4-((8-isopropoxy-7-(1H-pyrazol-4-yl)-[1,2,4]triazolo[1,5-c]pyrimidin-2-yl)amino)phenyl)sulfonyl)piperidin-3-yl)methyl)-2,2-dimethylpiperidin-4-yl)-1-methyl-1H-indazol-3-yl)dihydropyrimidine-2,4(1H,3H)-dione (103)

The rel-1-(6-((R)-1-(((R)-1-((3-fluoro-4-((8-isopropoxy-7-(1H-pyrazol-4-yl)-[1,2,4]triazolo[1,5-c]pyrimidin-2-yl)amino)phenyl) sulfonyl)piperidin-3-yl)methyl)-2,2-dimethylpiperidin-4-yl)-1-methyl-1H-indazol-3-yl)dihydropyrimidine-2,4(1H,3H)-dione (96, 32 mg) was separated by prep chiral SFC with the following conditions: column Name: chiral ART Amylose-SA column size: 4.6*100 mm, 3 μm co-solvent: methanol:dichloromethane=1:1 (0.2% DEA) gradient(B %): 50% back pressure: 110 bar flow: 2 ml/min temperature: 35; detector, UV 254 nm. The prepeak fractions were evaporated to dryness to afford rel-1-(6-((R)-1-(((R)-1-((3-fluoro-4-((8-isopropoxy-7-(1H-pyrazol-4-yl)-[1,2,4]triazolo[1,5-c]pyrimidin-2-yl)amino)phenyl)sulfonyl)piperidin-3-yl)methyl)-2,2-dimethylpiperidin-4-yl)-1-methyl-1H-indazol-3-yl)dihydropyrimidine-2,4(1H,3H)-dione (102, 7.7 mg, 25.00%) as a white solid. LCMS (ESI, m/z): [M+H]⁺ 868.3. The postpeak fractions were evaporated to dryness to afford rel-1-(6-((R)-1-(((S)-1-((3-fluoro-4-((8-isopropoxy-7-(1H-pyrazol-4-yl)-[1,2,4]triazolo[1,5-c]pyrimidin-2-yl)amino)phenyl)sulfonyl)piperidin-3-yl)methyl)-2,2-dimethylpiperidin-4-yl)-1-methyl-1H-indazol-3-yl)dihydropyrimidine-2,4(1H,3H)-dione (103, 6.5 mg, 20.97%) as a white solid. LCMS (ESI, m/z): [M+H]868.3.

### Step C. rel-1-(6-((R)-1-(((S)-1-((3-fluoro-4-((8-isopropoxy-7-(1H-pyrazol-4-yl)-[1,2,4]triazolo[1,5-c]pyrimidin-2-yl)amino)phenyl)sulfonyl)piperidin-3-yl)methyl)-2,2-dimethylpiperidin-4-yl)-1-methyl-1H-indazol-3-yl)dihydropyrimidine-2,4(1H,3H)-dione (104) and rel-1-(6-((R)-1-(((R)-1-((3-fluoro-4-((8-isopropoxy-7-(1H-pyrazol-4-yl)-[1,2,4]triazolo[1,5-c]pyrimidin-2-yl)amino)phenyl)sulfonyl)piperidin-3-yl)methyl)-2,2-dimethylpiperidin-4-yl)-1-methyl-1H-indazol-3-yl)dihydropyrimidine-2,4(1H,3H)-dione (105)

The rel-1-(6-((R)-1-(((S)-1-((3-fluoro-4-((8-isopropoxy-7-(1H-pyrazol-4-yl)-[1,2,4]triazolo[1,5-c]pyrimidin-2-yl)amino)phenyl)sulfonyl)piperidin-3-yl)methyl)-2,2-dimethylpiperidin-4-yl)-1-methyl-1H-indazol-3-yl)dihydropyrimidine-2,4(1H,3H)-dione (97, 44 mg) was purified by prep chiral HPLC with the following conditions: mobile phase:(Hex:MtBE=1:1)(0.1% DEA):methanol=85:15 Flow rate:1.67 ml/min; detector, UV 254 nm. The prepeak fractions were evaporated to dryness to afford rel-1-(6-((R)-1-(((S)-1-((3-fluoro-4-((8-isopropoxy-7-(1H-pyrazol-4-yl)-[1,2,4]triazolo[1,5-c]pyrimidin-2-yl)amino)phenyl)sulfonyl)piperidin-3-yl)methyl)-2,2-dimethylpiperidin-4-yl)-1-methyl-1H-indazol-3-yl)dihydropyrimidine-2,4(1H,3H)-dione (104, 15 mg, 37.13%) as a white solid. LCMS (ESI, m/z): [M+H]⁺ 868.3. The postpeak fractions were evaporated to dryness to afford rel-1-(6-((R)-1-(((R)-1-((3-fluoro-4-((8-isopropoxy-7-(1H-pyrazol-4-yl)-[1,2,4]triazolo[1,5-c]pyrimidin-2-yl)amino)phenyl)sulfonyl)piperidin-3-yl)methyl)-2,2-dimethylpiperidin-4-yl)-1-methyl-1H-indazol-3-yl)dihydropyrimidine-2,4(1H,3H)-dione (105, 15 mg, 37.28%) as a white solid. LCMS (ESI, m/z): [M+H]⁺ 868.3.

### Example 14. rel-(R)-1-(6-(4-(1-(3-((3-fluoro-4-((8-isopropoxy-7-(1H-pyrazol-4-yl)-[1,2,4]triazolo[1,5-c]pyrimidin-2-yl)amino)phenyl)sulfonyl)phenyl)ethyl)piperazin-1-yl)-1-methyl-1H-indazol-3-yl)dihydropyrimidine-2,4(1H,3H)-dione (98) and rel-(R)-1-(6-(4-(1-(3-((3-fluoro-4-((8-isopropoxy-7-(1H-pyrazol-4-yl)-[1,2,4]triazolo[1,5-c]pyrimidin-2-yl)amino)phenyl)sulfonyl)phenyl)ethyl)piperazin-1-yl)-1-methyl-1H-indazol-3-yl)dihydropyrimidine-2,4(1H,3H)-dione (99)

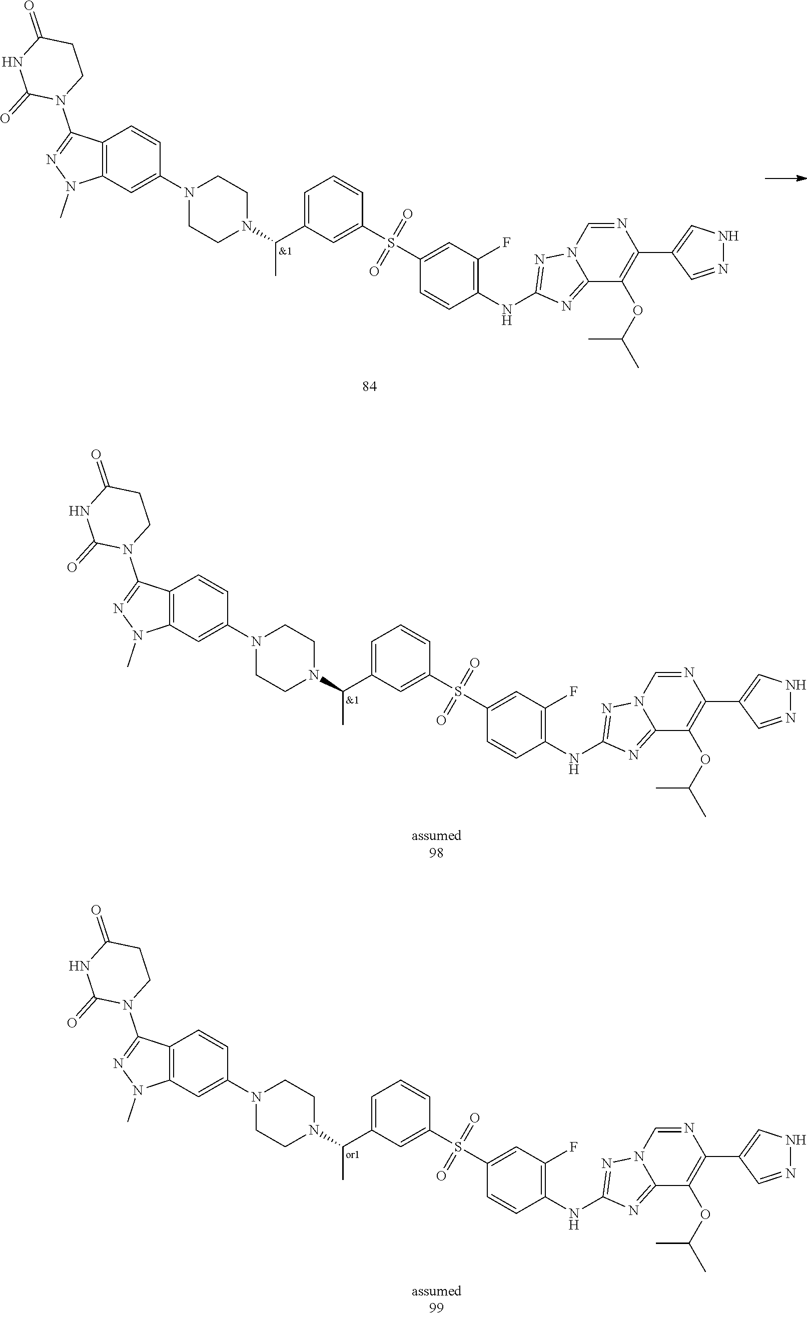 [SMILES: CC(C)Oc1c(-c2cn[nH]c2)ncn2nc(Nc3ccc(S(=O)(=O)c4cccc([C@H](C)N5CCN(c6ccc7c(N8CCC(=O)NC8=O)nn(C)c7c6)CC5)c4)cc3F)nc12 ; CC(C)Oc1c(-c2cn[nH]c2)ncn2nc(Nc3ccc(S(=O)(=O)c4cccc([C@@H](C)N5CCN(c6ccc7c(N8CCC(=O)NC8=O)nn(C)c7c6)CC5)c4)cc3F)nc12 ; CC(C)Oc1c(-c2cn[nH]c2)ncn2nc(Nc3ccc(S(=O)(=O)c4cccc([C@H](C)N5CCN(c6ccc7c(N8CCC(=O)NC8=O)nn(C)c7c6)CC5)c4)cc3F)nc12]

The rac-(R)-1-(6-(4-(1-(3-((3-fluoro-4-((8-isopropoxy-7-(1H-pyrazol-4-yl)-[1,2,4]triazolo[1,5-c]pyrimidin-2-yl)amino)phenyl)sulfonyl)phenyl)ethyl)piperazin-1-yl)-1-methyl-1H-indazol-3-yl)dihydropyrimidine-2,4(1H,3H)-dione (84, 40 mg, 0.047 mmol, 1 equiv) was separated by prep chiral HPLC, mobile phase A: methyl tert-butyl ether (10 mM NH₃-methanol), mobile phase B: methanol; Flow rate: 20 mL/min; gradient: isocratic 50; wave length: 306/252 nm; RT1(min): 12.5; RT2(min): 19.0; sample solvent: methanol: dichloromethane=2:1; injection volume: 1.5 mL; number of runs: 4. The prepeak fractions were evaporated to dryness to afford rel-(R)-1-(6-(4-(1-(3-((3-fluoro-4-((8-isopropoxy-7-(1H-pyrazol-4-yl)-[1,2,4]triazolo[1,5-c]pyrimidin-2-yl)amino)phenyl)sulfonyl)phenyl)ethyl)piperazin-1-yl)-1-methyl-1H-indazol-3-yl)dihydropyrimidine-2,4(1H,3H)-dione (98, 11.6 mg, 28.25%) as a white solid. LCMS (ESI, m/z): [M+H]⁺ 848.3. The postpeak fractions were evaporated to dryness to afford rel-(R)-1-(6-(4-(1-(3-((3-fluoro-4-((8-isopropoxy-7-(1H-pyrazol-4-yl)-[1,2,4]triazolo[1,5-c]pyrimidin-2-yl)amino)phenyl)sulfonyl)phenyl)ethyl)piperazin-1-yl)-1-methyl-1H-indazol-3-yl)dihydropyrimidine-2,4(1H,3H)-dione (99, 11.6 mg, 28.25%) as a white solid. LCMS (ESI, m/z): [M+H]⁺ 848.3.

### Example 15. rel-(R)-1-(6-(1-(1-(3-((3-fluoro-4-((8-isopropoxy-7-(1H-pyrazol-4-yl)-[1,2,4]triazolo[1,5-c]pyrimidin-2-yl)amino)phenyl)sulfonyl)phenyl)ethyl)piperidin-4-yl)-1-methyl-1H-indazol-3-yl)dihydropyrimidine-2,4(1H,3H)-dione (100) and rel-(R)-1-(6-(1-(1-(3-((3-fluoro-4-((8-isopropoxy-7-(1H-pyrazol-4-yl)-[1,2,4]triazolo[1,5-c]pyrimidin-2-yl)amino)phenyl)sulfonyl)phenyl)ethyl)piperidin-4-yl)-1-methyl-1H-indazol-3-yl)dihydropyrimidine-2,4(1H,3H)-dione (101)

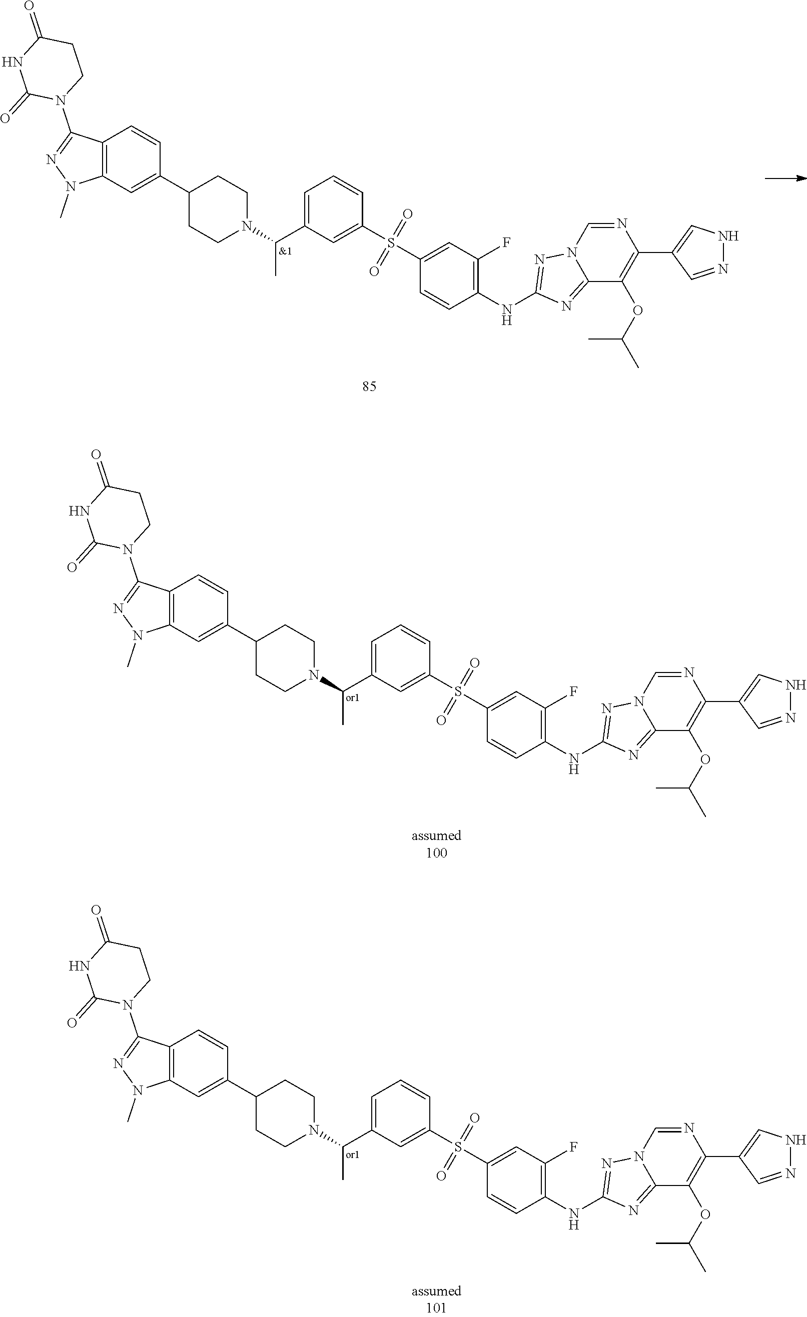 [SMILES: CC(C)Oc1c(-c2cn[nH]c2)ncn2nc(Nc3ccc(S(=O)(=O)c4cccc([C@H](C)N5CCC(c6ccc7c(N8CCC(=O)NC8=O)nn(C)c7c6)CC5)c4)cc3F)nc12 ; CC(C)Oc1c(-c2cn[nH]c2)ncn2nc(Nc3ccc(S(=O)(=O)c4cccc([C@@H](C)N5CCC(c6ccc7c(N8CCC(=O)NC8=O)nn(C)c7c6)CC5)c4)cc3F)nc12 ; CC(C)Oc1c(-c2cn[nH]c2)ncn2nc(Nc3ccc(S(=O)(=O)c4cccc([C@H](C)N5CCC(c6ccc7c(N8CCC(=O)NC8=O)nn(C)c7c6)CC5)c4)cc3F)nc12]

The racemate rac-1-(6-{1-[(1R)-1-[3-(3-fluoro-4-{[8-isopropoxy-7-(1H-pyrazol-4-yl)-[1,2,4]triazolo[1,5-c]pyrimidin-2-yl]amino}benzenesulfonyl)phenyl]ethyl]piperidin-4-yl}-1-methylindazol-3-yl)-1,3-diazinane-2,4-dione (70 mg, 0.083 mmol, 1 equiv) was purified by Prep-Chiral-HPLC with the following conditions (Column: CHIRALPAK AD-3, 3.0*100 mm, 3 μm; mobile phase A: Hex(10 mM NH₃-methanol), mobile phase B: isopropanol: acetonitrile=5:1. Flow rate: 40 mL/min; gradient: isocratic 50; wave length: 5 nm; RT1(min): 10.76; RT2(min): 16.272; sample solvent: acetonitrile; injection volume: 1.3 mL; number of runs: 200/304). The prepeak fractions were evaporated to dryness to afford rel-1-(6-{1-[(1R)-1-[3-(3-fluoro-4-{[8-isopropoxy-7-(1H-pyrazol-4-yl)-[1,2,4]triazolo[1,5-c]pyrimidin-2-yl]amino}benzenesulfonyl)phenyl]ethyl]piperidin-4-yl}-1-methylindazol-3-yl)-1,3-diazinane-2,4-dione (100, 24.4 mg, 34.23%) as a white solid. LCMS (ESI, m/z): [M+H]⁺ 847.3. The postpeak fractions were evaporated to dryness to afford rel-1-(6-{1-[(1R)-1-[3-(3-fluoro-4-{[8-isopropoxy-7-(1H-pyrazol-4-yl)-[1,2,4]triazolo[1,5-c]pyrimidin-2-yl]amino}benzenesulfonyl)phenyl]ethyl]piperidin-4-yl}-1-methylindazol-3-yl)-1,3-diazinane-2,4-dione (101, 21.7 mg, 29.92%) as a white solid. LCMS (ESI, m/z): [M+H]⁺ 847.3.

### Example 16. rel-(R)-1-(6-(4-(3-((3-fluoro-4-((8-isopropoxy-7-(1H-pyrazol-4-yl)-[1,2,4]triazolo[1,5-c]pyrimidin-2-yl)amino)phenyl)sulfonyl)benzyl)-3-isopropylpiperazin-1-yl)-1-methyl-1H-indazol-3-yl)dihydropyrimidine-2,4(1H,3H)-dione (106) and rel-(R)-1-(6-(4-(3-((3-fluoro-4-((8-isopropoxy-7-(1H-pyrazol-4-yl)-[1,2,4]triazolo[1,5-c]pyrimidin-2-yl)amino)phenyl)sulfonyl)benzyl)-3-isopropylpiperazin-1-yl)-1-methyl-1H-indazol-3-yl)dihydropyrimidine-2,4(1H,3H)-dione (107)

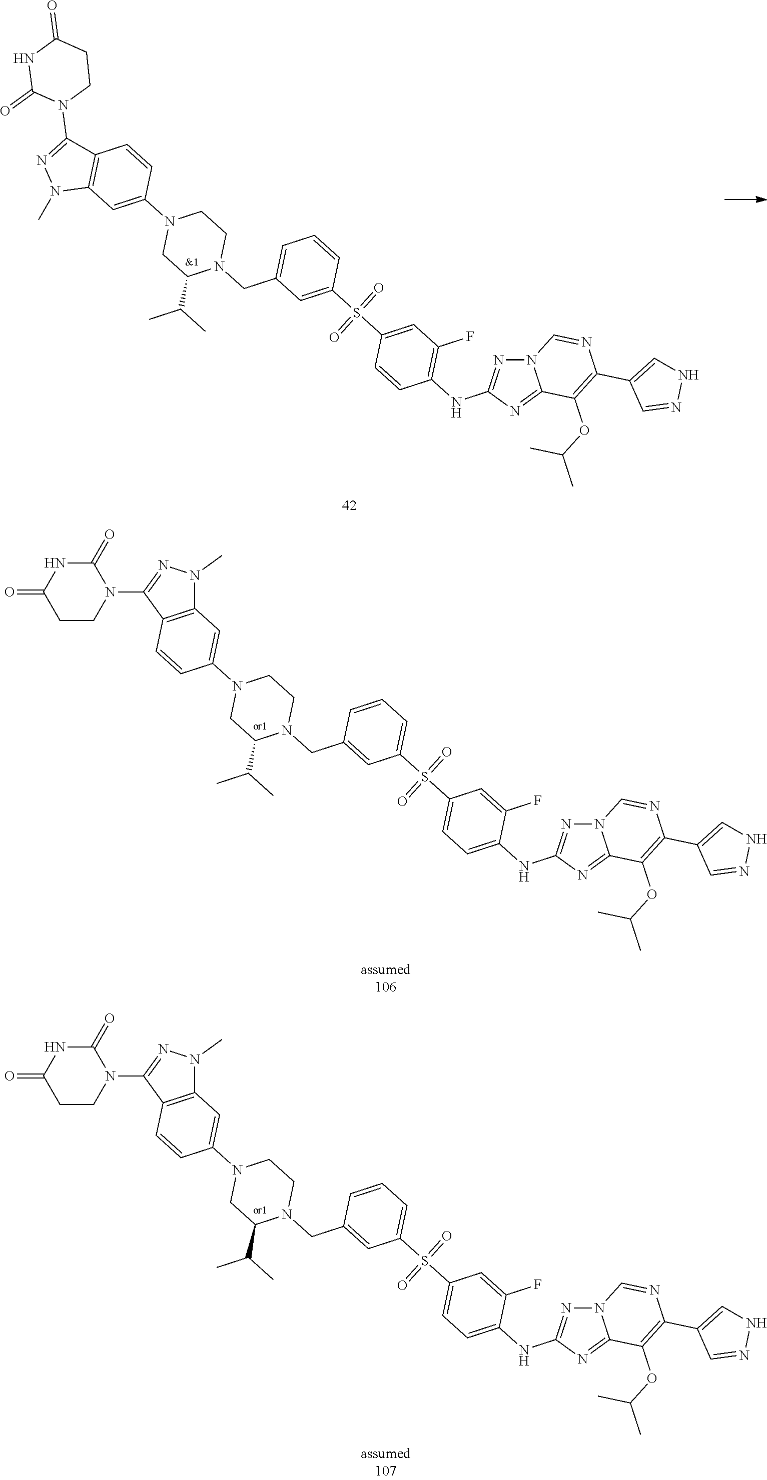 [SMILES: CC(C)Oc1c(-c2cn[nH]c2)ncn2nc(Nc3ccc(S(=O)(=O)c4cccc(CN5CCN(c6ccc7c(N8CCC(=O)NC8=O)nn(C)c7c6)C[C@H]5C(C)C)c4)cc3F)nc12 ; CC(C)Oc1c(-c2cn[nH]c2)ncn2nc(Nc3ccc(S(=O)(=O)c4cccc(CN5CCN(c6ccc7c(N8CCC(=O)NC8=O)nn(C)c7c6)C[C@H]5C(C)C)c4)cc3F)nc12 ; CC(C)Oc1c(-c2cn[nH]c2)ncn2nc(Nc3ccc(S(=O)(=O)c4cccc(CN5CCN(c6ccc7c(N8CCC(=O)NC8=O)nn(C)c7c6)C[C@@H]5C(C)C)c4)cc3F)nc12]

The rac-(R)-1-(6-(4-(3-((3-fluoro-4-((8-isopropoxy-7-(1H-pyrazol-4-yl)-[1,2,4]triazolo[1,5-c]pyrimidin-2-yl)amino)phenyl)sulfonyl)benzyl)-3-isopropylpiperazin-1-yl)-1-methyl-1H-indazol-3-yl)dihydropyrimidine-2,4(1H,3H)-dione (42, 30 mg) was purified by chiral-HPLC with the following conditions: Column: CHIRALPAK IA, 3*25 cm, 5 μm; mobile phase A: Hex: methyl tert-butyl ether=1:1(0.5% 2M NH₃-methanol), mobile phase B: methanol; Flow rate: 40 mL/min; gradient: isocratic 30; wave length: 306/210 nm; RT1(min): 26.51; RT2 (min): 33.69; sample solvent: methanol: dichloromethane=1:1--HPLC; number of runs: 2. The prepeak fractions were evaporated to dryness to afford rel-1-{6-[(3R)-4-{[3-(3-fluoro-4-{[8-isopropoxy-7-(1H-pyrazol-4-yl)-[1,2,4]triazolo[1,5-c]pyrimidin-2-yl]amino}benzenesulfonyl)phenyl]methyl}-3-isopropylpiperazin-1-yl]-1-methylindazol-3-yl}-1,3-diazinane-2,4-dione (106, 5.2 mg, 16.50%) as a white solid. LCMS (ESI, m/z): [M+H]⁺ 876.3. The postpeak fractions were evaporated to dryness to afford rel-1-{6-[(3R)-4-{[3-(3-fluoro-4-{[8-isopropoxy-7-(1H-pyrazol-4-yl)-[1,2,4]triazolo[1,5-c]pyrimidin-2-yl]amino}benzenesulfonyl)phenyl]ethyl}-3-isopropylpiperazin-1-yl]-1-methylindazol-3-yl}-1,3-diazinane-2,4-dione (107, 14.3 mg, 47.38%) as a white solid. LCMS (ESI, m/z): [M+H]⁺ 876.3.

### Example 17. rel-1-{6-[(2R)-4-{[3-(3-fluoro-4-{[8-isopropoxy-7-(1H-pyrazol-4-yl)-[1,2,4]triazolo[1,5-c]pyrimidin-2-yl]amino}benzenesulfonyl)phenyl]methyl}-2-isopropylpiperazin-1-yl]-1-methylindazol-3-yl}-1,3-diazinane-2,4-dione (108) and rel-1-{6-[(2R)-4-{[3-(3-fluoro-4-{[8-isopropoxy-7-(1H-pyrazol-4-yl)-[1,2,4]triazolo1,5-c]pyrimidin-2-yl]amino}benzenesulfonyl)phenyl]methyl}-2-isopropylpiperazin-1-yl]-1-methylindazol-3-yl}-1,3-diazinane-2,4-dione (109)

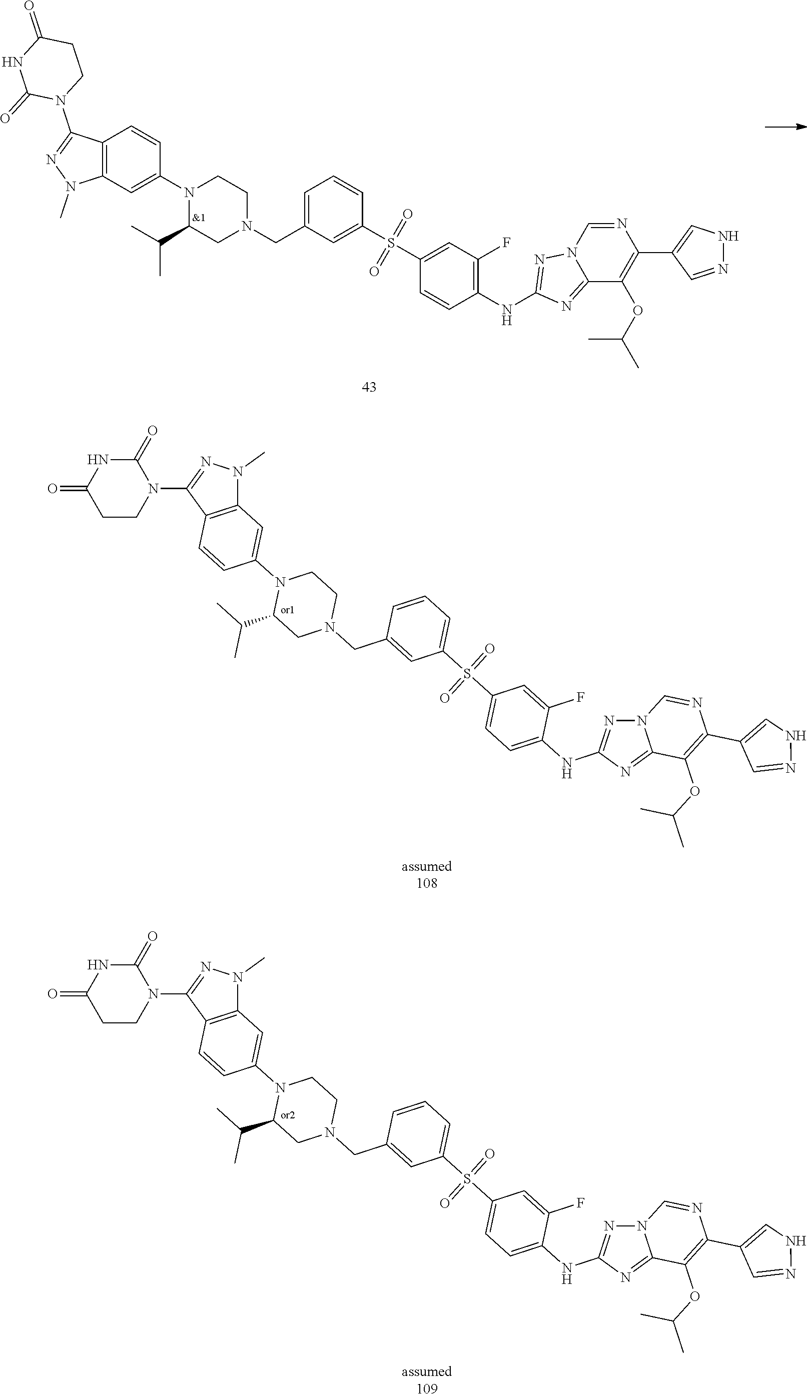 [SMILES: CC(C)Oc1c(-c2cn[nH]c2)ncn2nc(Nc3ccc(S(=O)(=O)c4cccc(CN5CCN(c6ccc7c(N8CCC(=O)NC8=O)nn(C)c7c6)[C@H](C(C)C)C5)c4)cc3F)nc12 ; CC(C)Oc1c(-c2cn[nH]c2)ncn2nc(Nc3ccc(S(=O)(=O)c4cccc(CN5CCN(c6ccc7c(N8CCC(=O)NC8=O)nn(C)c7c6)[C@@H](C(C)C)C5)c4)cc3F)nc12 ; CC(C)Oc1c(-c2cn[nH]c2)ncn2nc(Nc3ccc(S(=O)(=O)c4cccc(CN5CCN(c6ccc7c(N8CCC(=O)NC8=O)nn(C)c7c6)[C@H](C(C)C)C5)c4)cc3F)nc12]

The rac-1-{6-[(2R)-4-{[3-(3-fluoro-4-{[8-isopropoxy-7-(1H-pyrazol-4-yl)-[1, 2, 4]triazolo[1,5-c]pyrimidin-2-yl]amino}benzenesulfonyl)phenyl]methyl}-2-isopropylpiperazin-1-yl]-1-methylindazol-3-yl}-1,3-diazinane-2,4-dione (43, 20 mg) was purified by prep chiral HPLC with following conditions: Column: CHIRALPAK IA, 3*25 cm, 5 μm; mobile phase A: methyl tert-butyl ether (10 mM NH3-methanol), mobile phase B: methanol; sample solvent: methanol:dichloromethane=1:1. The prepeak fractions were evaporated to dryness to afford rel-1-{6-[(2R)-4-{[3-(3-fluoro-4-{[8-isopropoxy-7-(1H-pyrazol-4-yl)-[1,2,4]triazolo[1,5-c]pyrimidin-2-yl]amino}benzenesulfonyl)phenyl]methyl}-2-isopropylpiperazin-1-yl]-1-methylindazol-3-yl}-1,3-diazinane-2,4-dione (108, 6.5 mg, 32.21%) as a white solid. LCMS (ESI, m/z): [M+H]⁺ 876.35. The postpeak fractions were evaporated to dryness to afford rel-1-{6-[(2R)-4-{[3-(3-fluoro-4-{[8-isopropoxy-7-(1H-pyrazol-4-yl)-[1,2,4]triazolo[1,5-c]pyrimidin-2-yl]amino}benzenesulfonyl)phenyl]methyl}-2-isopropylpiperazin-1-yl]-1-methylindazol-3-yl}-1,3-diazinane-2,4-dione (109, 6.5 mg, 32.21%) as a white solid. LCMS (ESI, m/z): [M+H]⁺ 876.35.

### Example 18. 3-{1-oxo-5-[(1s,3s)-3-{[3-(3-fluoro-4-{[8-isopropoxy-7-(1H-pyrazol-4-yl)-[1,2,4]triazolo[1,5-c]pyrimidin-2-yl]amino}benzenesulfonyl)phenyl]methyl}cyclobutyl]-3H-isoindol-2-yl}piperidine-2,6-dione (29)

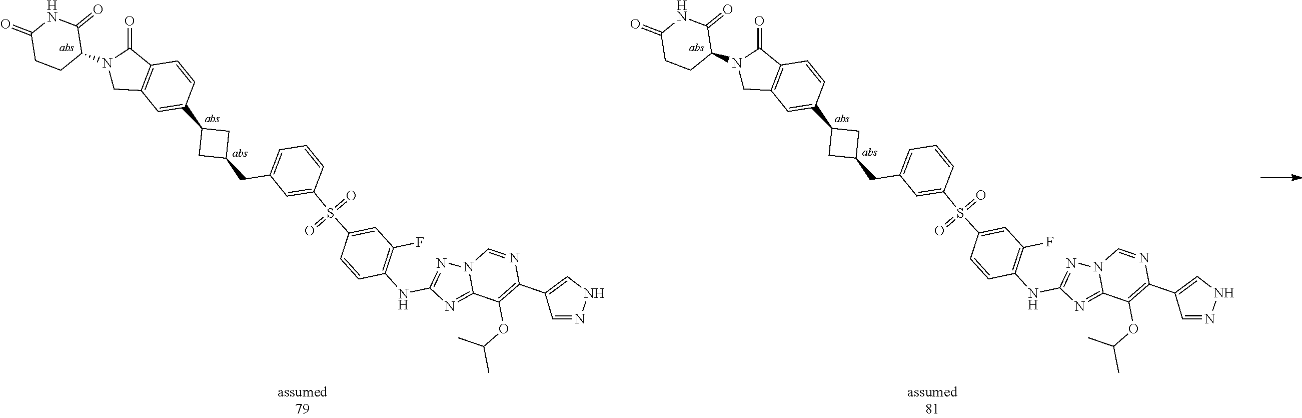 [SMILES: CC(C)Oc1c(-c2cn[nH]c2)ncn2nc(Nc3ccc(S(=O)(=O)c4cccc(C[C@H]5C[C@@H](c6ccc7c(c6)CN([C@@H]6CCC(=O)NC6=O)C7=O)C5)c4)cc3F)nc12 ; CC(C)Oc1c(-c2cn[nH]c2)ncn2nc(Nc3ccc(S(=O)(=O)c4cccc(C[C@H]5C[C@@H](c6ccc7c(c6)CN([C@H]6CCC(=O)NC6=O)C7=O)C5)c4)cc3F)nc12]

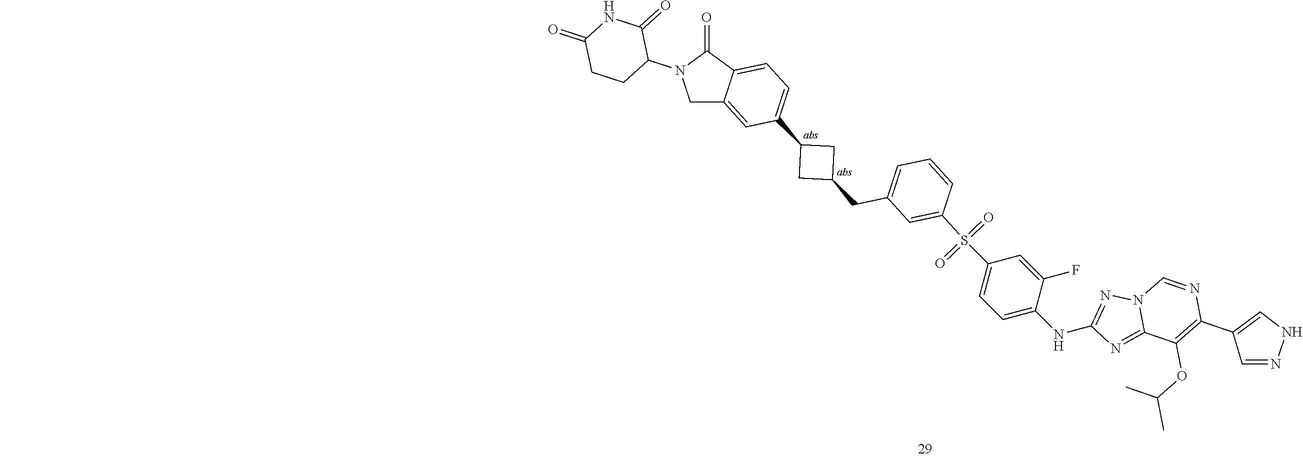 [SMILES: CC(C)Oc1c(-c2cn[nH]c2)ncn2nc(Nc3ccc(S(=O)(=O)c4cccc(C[C@H]5C[C@@H](c6ccc7c(c6)CN(C6CCC(=O)NC6=O)C7=O)C5)c4)cc3F)nc12]

To a stirred solution of (3R)-3-{1-oxo-5-[(1s,3s)-3-{[3-(3-fluoro-4-{[8-isopropoxy-7-(1H-pyrazol-4-yl)-[1,2,4]triazolo[1,5-c]pyrimidin-2-yl]amino}benzenesulfonyl)phenyl]methyl}cyclobutyl]-3H-isoindol-2-yl}piperidine-2,6-dione (79, 4 mg) in acetonitrile was added (3S)-3-{1-oxo-5-[(1s,3s)-3-{[3-(3-fluoro-4-{[8-isopropoxy-7-(1H-pyrazol-4-yl)-[1,2,4]triazolo[1,5-c]pyrimidin-2-yl]amino}benzenesulfonyl)phenyl]methyl}cyclobutyl]-3H-isoindol-2-yl}piperidine-2,6-dione (81, 4 mg). The resulting mixture was stirred for 1 hours at 25° C. under nitrogen atmosphere. The mixture was concentrated under reduced pressure to get 8 mg 3-{1-oxo-5-[(1s,3s)-3-{[3-(3-fluoro-4-{[8-isopropoxy-7-(1H-pyrazol-4-yl)-[1,2,4]triazolo[1,5-c]pyrimidin-2-yl]amino}benzenesulfonyl)phenyl]methyl}cyclobutyl]-3H-isoindol-2-yl}piperidine-2,6-dione (29, 8 mg, 199.20%) as a white solid. LCMS (ESI, m/z): [M+H]⁺ 804.3.

### Example 19. rac-N-[(3R)-2,6-dioxopiperidin-3-yl]-4-(3-{[3-(3-fluoro-4-{[8-isopropoxy-7-(1H-pyrazol-4-yl)-[1,2,4]triazolo[1,5-c]pyrimidin-2-yl]amino}benzenesulfonyl)phenyl]methyl}cyclobutyl)benzamide (32)

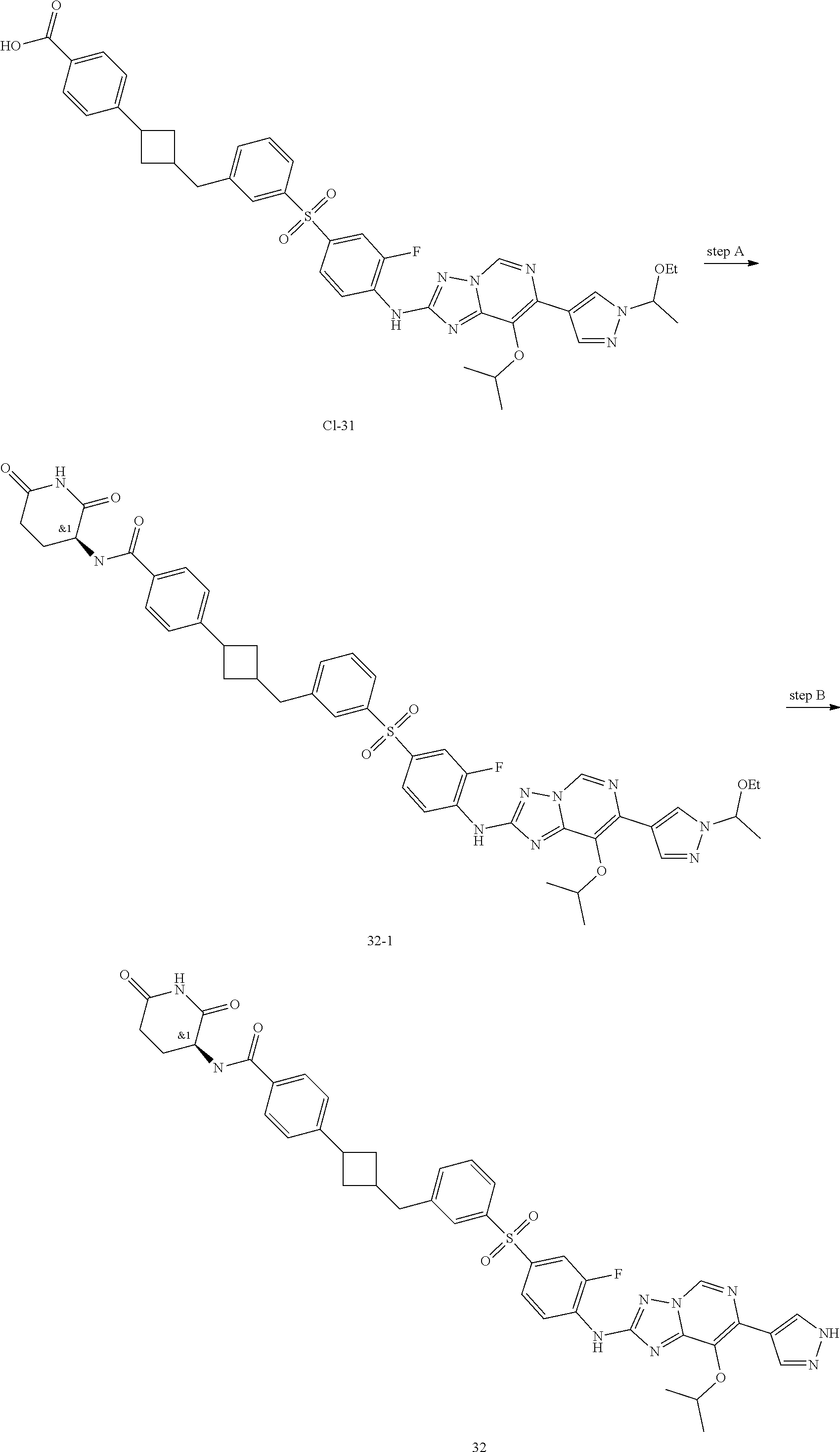

### Step A. N-[(3S)-2,6-dioxopiperidin-3-yl]-4-[3-({3-[4-({7-[1-(1-ethoxyethyl)pyrazol-4-yl]-8-isopropoxy-[1,2,4]triazolo[1,5-c]pyrimidin-2-yl}amino)-3-fluorobenzenesulfonyl]phenyl}methyl)cyclobutyl]benzamide (32-1)

To a stirred solution of 4-[3-({3-[4-({7-[1-(1-ethoxyethyl)pyrazol-4-yl]-8-isopropoxy-[1,2,4]triazolo[1,5-c]pyrimidin-2-yl}amino)-3-fluorobenzenesulfonyl]phenyl}methyl)cyclobutyl]benzoic acid (CI-31, 300 mg, 0.398 mmol, 1 equiv) and (3S)-3-aminopiperidine-2,6-dione hydrochloride (98.25 mg, 0.597 mmol, 1.5 equiv) in N,N-dimethylformamide (5 mL) were added HATU (226.98 mg, 0.597 mmol, 1.5 equiv) and N,N-diisopropylethylamine (514.35 mg, 3.980 mmol, 10 equiv) in portions at room temperature under nitrogen atmosphere. The resulting mixture was stirred for 4 h at room temperature under nitrogen atmosphere. Desired product could be detected by LCMS. The residue was purified by reversed-phase flash chromatography with the following conditions: column, C18 silica gel; mobile phase, acetonitrile in water (0.1% formic acid), 10% to 67% gradient in 10 min; detector, UV 254 nm. The resulting mixture was concentrated under reduced pressure to afford N-[(3S)-2,6-dioxopiperidin-3-yl]-4-[3-({3-[4-({7-[1-(1-ethoxyethyl)pyrazol-4-yl]-8-isopropoxy-[1,2,4]triazolo[1,5-c]pyrimidin-2-yl}amino)-3-fluorobenzenesulfonyl]phenyl}methyl)cyclobutyl]benzamide (32-1, 200 mg, 50.61%) as a white solid. LCMS (ES, m/z): [M+H]⁺ 864.3.

### Step B. rac-N-[(3R)-2,6-dioxopiperidin-3-yl]-4-(3-{[3-(3-fluoro-4-{[8-isopropoxy-7-(1H-pyrazol-4-yl)-[1,2,4]triazolo[1,5-c]pyrimidin-2-yl]amino}benzenesulfonyl)phenyl]methyl}cyclobutyl)benzamide (32)

To a stirred solution of rac-N-[(3R)-2,6-dioxopiperidin-3-yl]-4-[3-({3-[4-({7-[1-(1-ethoxyethyl)pyrazol-4-yl]-8-isopropoxy-[1,2,4]triazolo[1,5-c]pyrimidin-2-yl}amino)-3-fluorobenzenesulfonyl]phenyl}methyl)cyclobutyl]benzamide (32-1, 50 mg, 0.058 mmol, 1 equiv) in dichloromethane (3 mL) was added trifluoroacetic acid (0.5 mL, 0.005 mmol, 0.05 equiv) dropwise at 0° C. under nitrogen atmosphere. The resulting mixture was stirred for 30 min at 0° C. under nitrogen atmosphere. Desired product could be detected by LCMS. The mixture was neutralized to pH 7 with trimethylamine (1 mL). The resulting mixture was concentrated under reduced pressure. The residue was purified by reversed-phase flash chromatography with the following conditions: column, C18 silica gel; mobile phase, acetonitrile in water (10 mmol/L ammonium bicarbonate), 10% to 69% gradient in 10 min; detector, UV 254 nm. The resulting mixture was concentrated under reduced pressure to afford rac-N-[(3R)-2,6-dioxopiperidin-3-yl]-4-(3-{[3-(3-fluoro-4-{[8-isopropoxy-7-(1H-pyrazol-4-yl)-[1,2,4]triazolo[1,5-c]pyrimidin-2-yl]amino}benzenesulfonyl)phenyl]methyl}cyclobutyl)benzamide (32, 15.4 mg, 32.66%) as a white solid. LCMS (ES, m/z): [M+H]⁺ 792.1.

Compounds 1 to 5, 40, 49 to 52 were prepared as described for Example 1. Compounds 6, 7, 19 were prepared as described for Example 2. Compounds 8 to 13, 20, 25, 26, 38, 39, 41 to 48, 73, 78 were prepared as described for Example 3. Compounds 14 to 18, 24, 27, 28, 37, 23, 54, 57, 61 to 72, 86 to 88, 91 to 93 were prepared as described for Example 4. Compounds 21 to 23, 36, 74, 76 were prepared as described for Example 5. Compounds 31, 33 and 77 were prepared as described for Example 6. Compounds 34, 35, 75 were prepared as described for Example 7. Compounds 55, 58 to 60 were prepared as described for Example 8. Compounds 83 to 85 were prepared as described for Example 9 from common intermediates.

Chiral compounds 79 to 82 were separated as described for Example 10. Chiral compounds 89 and 90 were separated as described for Example 11. Chiral compounds 94 and 95 were separated as described for Example 12. Chiral compounds 96, 97, 102 to 105 were separated as described for Example 13. Chiral compounds 98 and 99 were separated as described for Example 14. Chiral compounds 100 and 101 were separated as described for Example 15. Chiral compounds 106 and 107 were separated as described for Example 16. Chiral compounds 108 and 109 were separated as described for Example 17. Compounds 29 and 30 were prepared as described for Example 18. Compound 32 was prepared as described for Example 19 from common intermediates.

TABLE 1
Structure, physicochemical data for compounds 1 to 109

| Cpd | |
|---|---|
| No. | Structure |
| 1 | 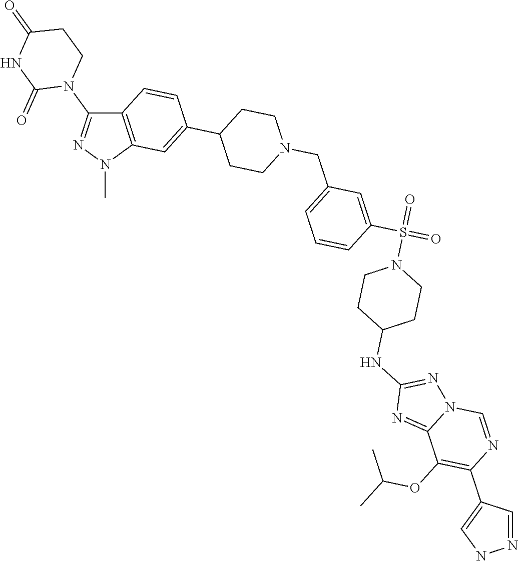 [SMILES: CC(C)Oc1c(-c2cn[nH]c2)ncn2nc(NC3CCN(S(=O)(=O)c4cccc(CN5CCC(c6ccc7c(N8CCC(=O)NC8=O)nn(C)c7c6)CC5)c4)CC3)nc12] |
| 2 | 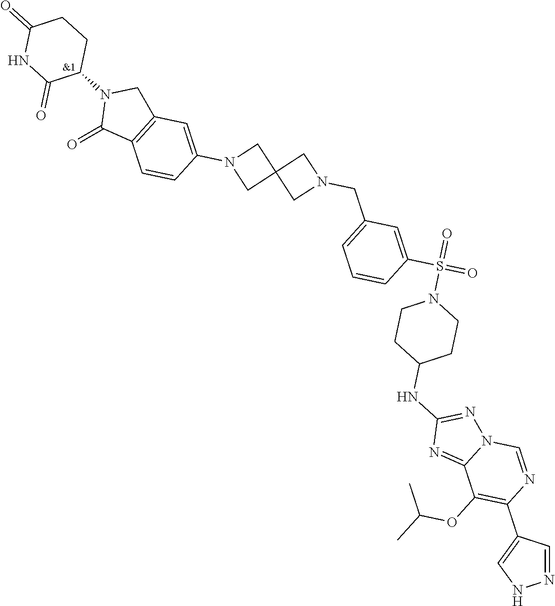 [SMILES: CC(C)Oc1c(-c2cn[nH]c2)ncn2nc(NC3CCN(S(=O)(=O)c4cccc(CN5CC6(C5)CN(c5ccc7c(c5)CN([C@H]5CCC(=O)NC5=O)C7=O)C6)c4)CC3)nc12] |
| 3 | 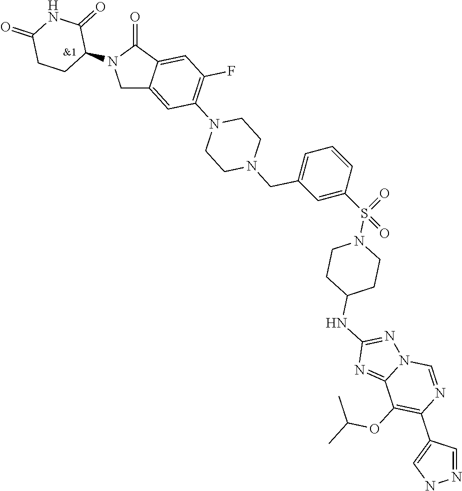 [SMILES: CC(C)Oc1c(-c2cn[nH]c2)ncn2nc(NC3CCN(S(=O)(=O)c4cccc(CN5CCN(c6cc7c(cc6F)C(=O)N([C@H]6CCC(=O)NC6=O)C7)CC5)c4)CC3)nc12] |
| 4 | 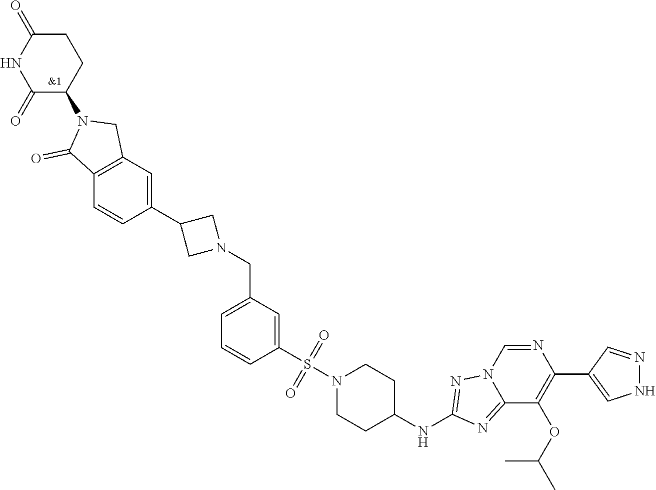 [SMILES: CC(C)Oc1c(-c2cn[nH]c2)ncn2nc(NC3CCN(S(=O)(=O)c4cccc(CN5CC(c6ccc7c(c6)CN([C@@H]6CCC(=O)NC6=O)C7=O)C5)c4)CC3)nc12] |
| 5 | 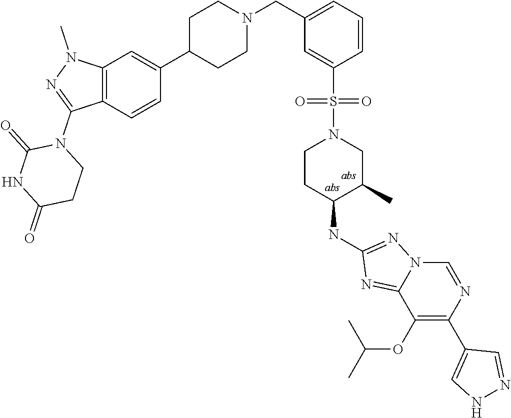 [SMILES: CC(C)Oc1c(-c2cn[nH]c2)ncn2nc(N[C@H]3CCN(S(=O)(=O)c4cccc(CN5CCC(c6ccc7c(N8CCC(=O)NC8=O)nn(C)c7c6)CC5)c4)C[C@H]3C)nc12] |
| 6 | 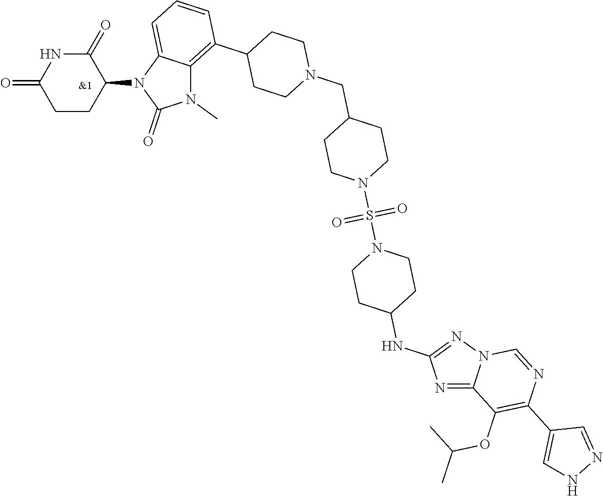 [SMILES: CC(C)Oc1c(-c2cn[nH]c2)ncn2nc(NC3CCN(S(=O)(=O)N4CCC(CN5CCC(c6cccc7c6n(C)c(=O)n7[C@H]6CCC(=O)NC6=O)CC5)CC4)CC3)nc12] |
| 7 | 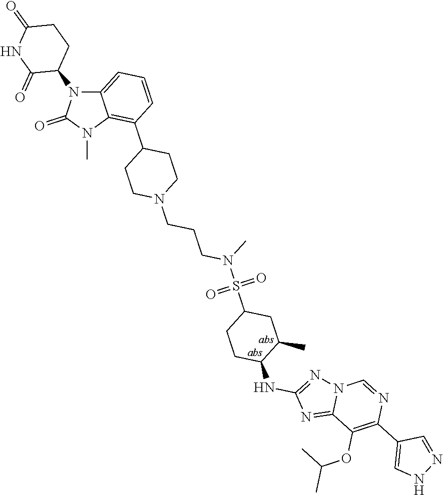 [SMILES: CC(C)Oc1c(-c2cn[nH]c2)ncn2nc(N[C@H]3CCC(S(=O)(=O)N(C)CCCN4CCC(c5cccc6c5n(C)c(=O)n6[C@@H]5CCC(=O)NC5=O)CC4)C[C@H]3C)nc12] |
| 8 | 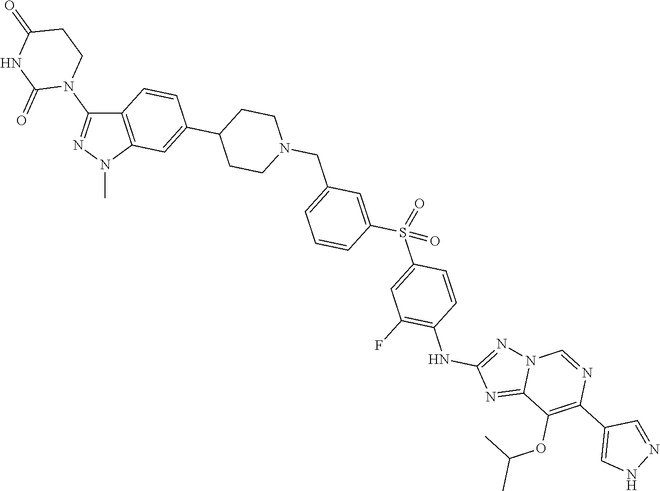 [SMILES: CC(C)Oc1c(-c2cn[nH]c2)ncn2nc(Nc3ccc(S(=O)(=O)c4cccc(CN5CCC(c6ccc7c(N8CCC(=O)NC8=O)nn(C)c7c6)CC5)c4)cc3F)nc12] |
| 9 | 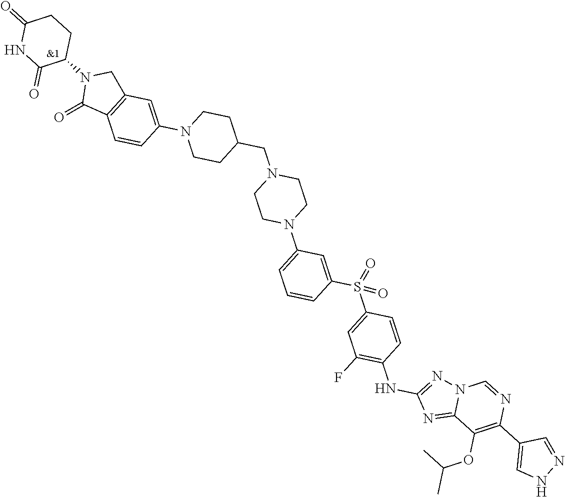 [SMILES: CC(C)Oc1c(-c2cn[nH]c2)ncn2nc(Nc3ccc(S(=O)(=O)c4cccc(N5CCN(CC6CCN(c7ccc8c(c7)CN([C@H]7CCC(=O)NC7=O)C8=O)CC6)CC5)c4)cc3F)nc12] |
| 10 | 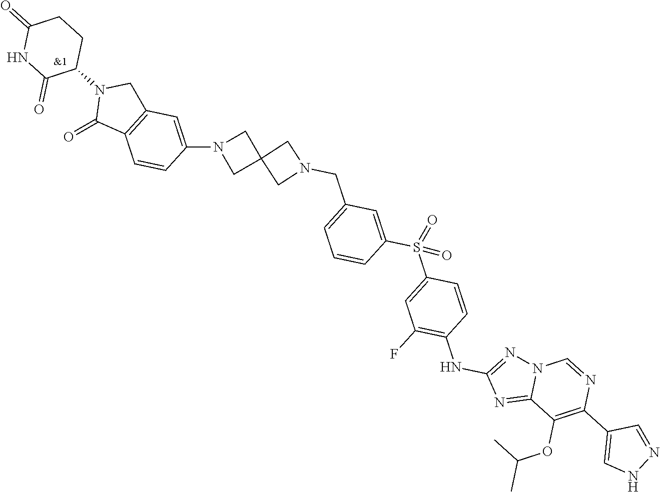 [SMILES: CC(C)Oc1c(-c2cn[nH]c2)ncn2nc(Nc3ccc(S(=O)(=O)c4cccc(CN5CC6(C5)CN(c5ccc7c(c5)CN([C@H]5CCC(=O)NC5=O)C7=O)C6)c4)cc3F)nc12] |
| 11 | 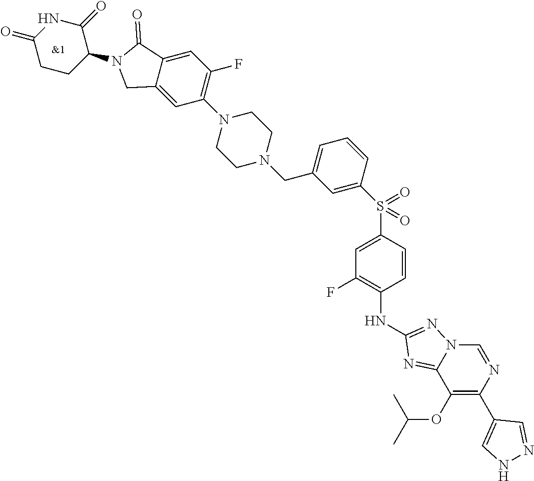 [SMILES: CC(C)Oc1c(-c2cn[nH]c2)ncn2nc(Nc3ccc(S(=O)(=O)c4cccc(CN5CCN(c6cc7c(cc6F)C(=O)N([C@H]6CCC(=O)NC6=O)C7)CC5)c4)cc3F)nc12] |
| 12 | 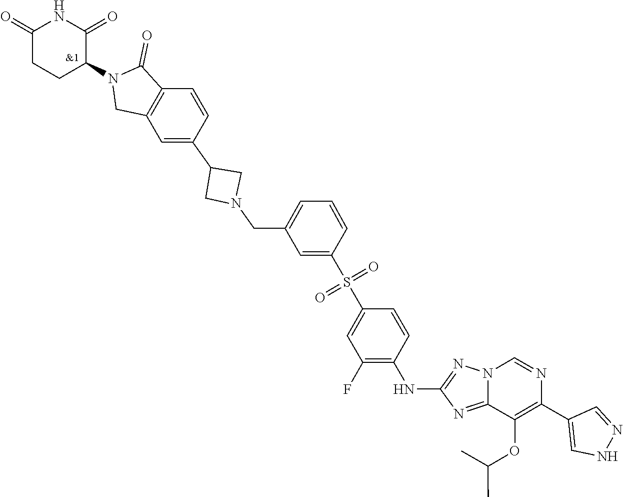 [SMILES: CC(C)Oc1c(-c2cn[nH]c2)ncn2nc(Nc3ccc(S(=O)(=O)c4cccc(CN5CC(c6ccc7c(c6)CN([C@H]6CCC(=O)NC6=O)C7=O)C5)c4)cc3F)nc12] |
| 13 | 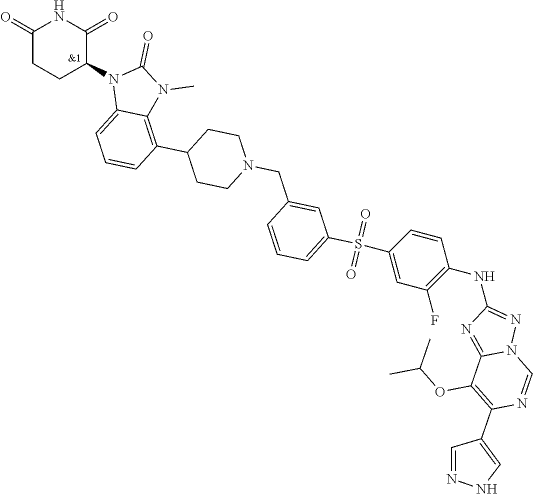 [SMILES: CC(C)Oc1c(-c2cn[nH]c2)ncn2nc(Nc3ccc(S(=O)(=O)c4cccc(CN5CCC(c6cccc7c6n(C)c(=O)n7[C@H]6CCC(=O)NC6=O)CC5)c4)cc3F)nc12] |
| 14 | 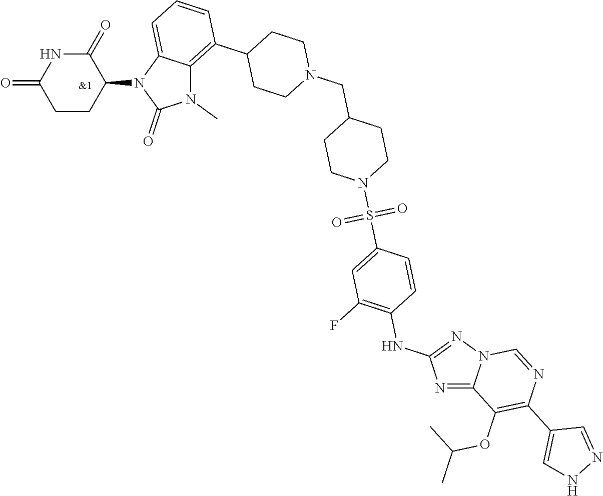 [SMILES: CC(C)Oc1c(-c2cn[nH]c2)ncn2nc(Nc3ccc(S(=O)(=O)N4CCC(CN5CCC(c6cccc7c6n(C)c(=O)n7[C@H]6CCC(=O)NC6=O)CC5)CC4)cc3F)nc12] |
| 15 | 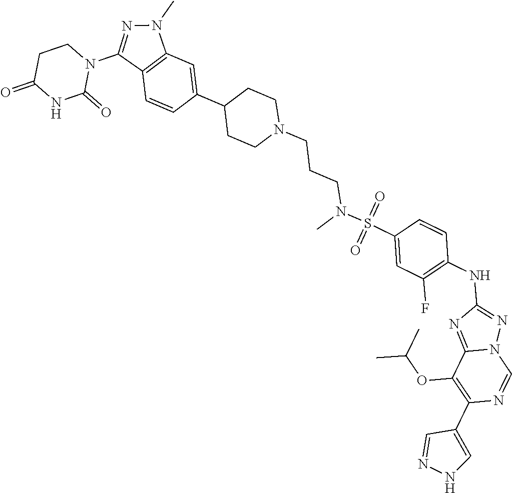 [SMILES: CC(C)Oc1c(-c2cn[nH]c2)ncn2nc(Nc3ccc(S(=O)(=O)N(C)CCCN4CCC(c5ccc6c(N7CCC(=O)NC7=O)nn(C)c6c5)CC4)cc3F)nc12] |
| 16 | 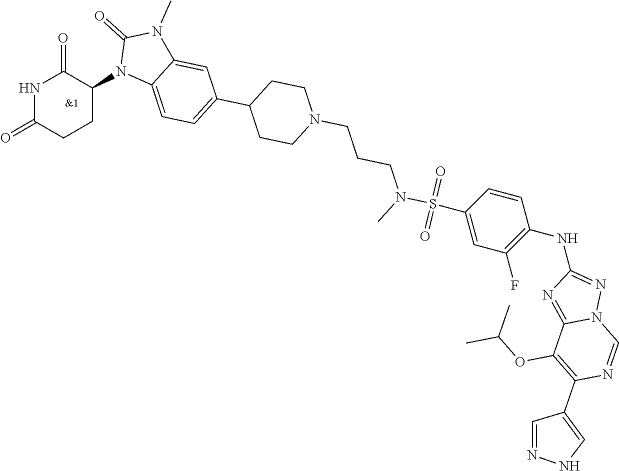 [SMILES: CC(C)Oc1c(-c2cn[nH]c2)ncn2nc(Nc3ccc(S(=O)(=O)N(C)CCCN4CCC(c5ccc6c(c5)n(C)c(=O)n6[C@H]5CCC(=O)NC5=O)CC4)cc3F)nc12] |
| 17 | 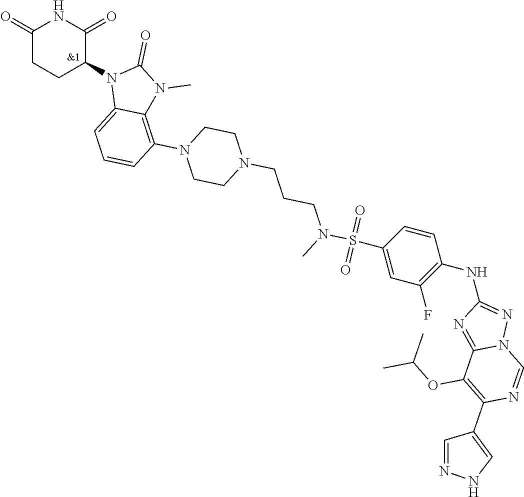 [SMILES: CC(C)Oc1c(-c2cn[nH]c2)ncn2nc(Nc3ccc(S(=O)(=O)N(C)CCCN4CCN(c5cccc6c5n(C)c(=O)n6[C@H]5CCC(=O)NC5=O)CC4)cc3F)nc12] |
| 18 | 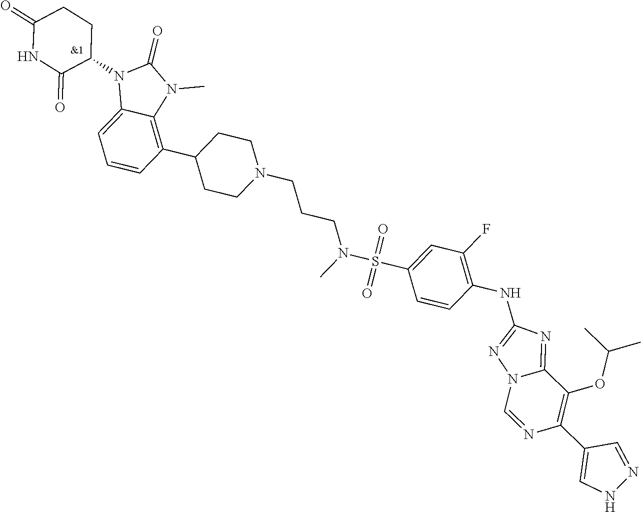 [SMILES: CC(C)Oc1c(-c2cn[nH]c2)ncn2nc(Nc3ccc(S(=O)(=O)N(C)CCCN4CCC(c5cccc6c5n(C)c(=O)n6[C@H]5CCC(=O)NC5=O)CC4)cc3F)nc12] |
| 19 | 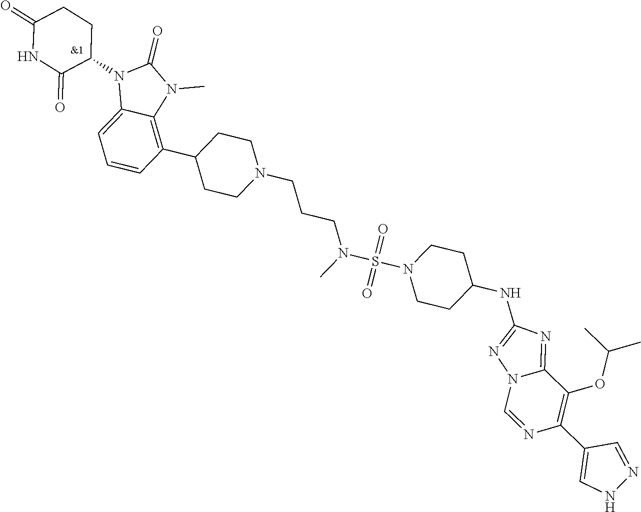 [SMILES: CC(C)Oc1c(-c2cn[nH]c2)ncn2nc(NC3CCN(S(=O)(=O)N(C)CCCN4CCC(c5cccc6c5n(C)c(=O)n6[C@H]5CCC(=O)NC5=O)CC4)CC3)nc12] |
| 20 | 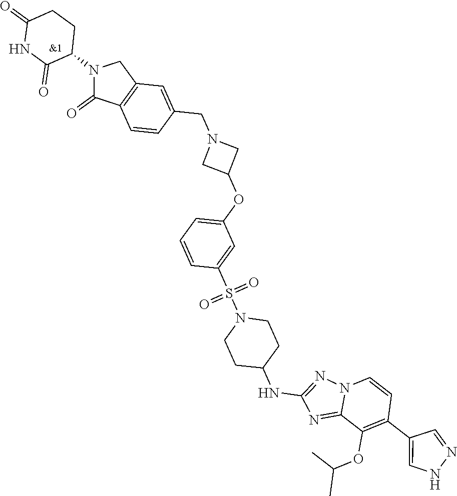 [SMILES: CC(C)Oc1c(-c2cn[nH]c2)ccn2nc(NC3CCN(S(=O)(=O)c4cccc(OC5CN(Cc6ccc7c(c6)CN([C@H]6CCC(=O)NC6=O)C7=O)C5)c4)CC3)nc12] |
| 21 | 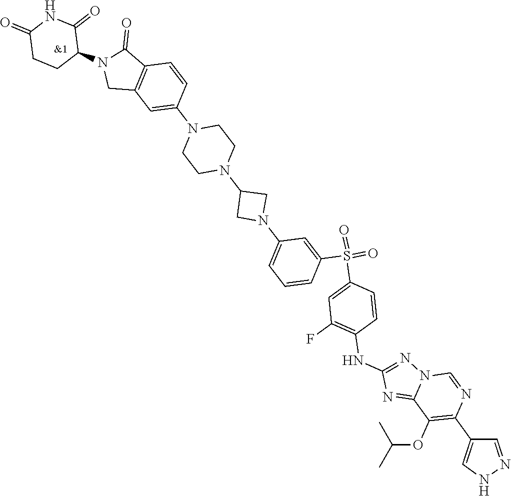 [SMILES: CC(C)Oc1c(-c2cn[nH]c2)ncn2nc(Nc3ccc(S(=O)(=O)c4cccc(N5CC(N6CCN(c7ccc8c(c7)CN([C@H]7CCC(=O)NC7=O)C8=O)CC6)C5)c4)cc3F)nc12] |
| 22 | 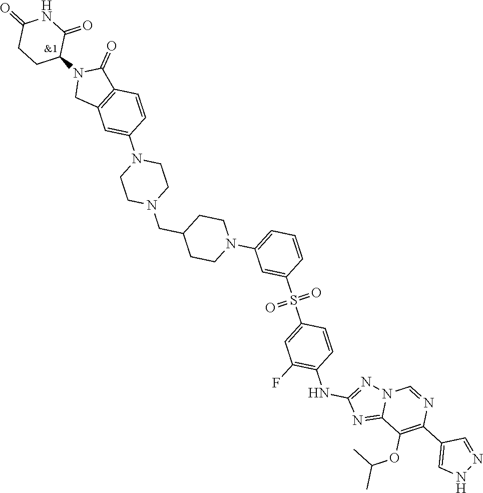 [SMILES: CC(C)Oc1c(-c2cn[nH]c2)ncn2nc(Nc3ccc(S(=O)(=O)c4cccc(N5CCC(CN6CCN(c7ccc8c(c7)CN([C@H]7CCC(=O)NC7=O)C8=O)CC6)CC5)c4)cc3F)nc12] |
| 23 | 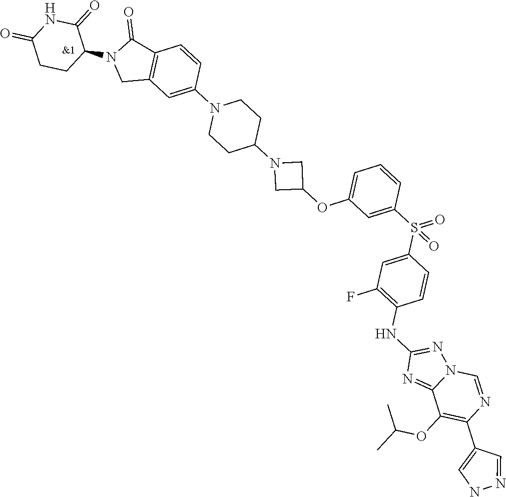 [SMILES: CC(C)Oc1c(-c2cn[nH]c2)ncn2nc(Nc3ccc(S(=O)(=O)c4cccc(OC5CN(C6CCN(c7ccc8c(c7)CN([C@H]7CCC(=O)NC7=O)C8=O)CC6)C5)c4)cc3F)nc12] |
| 24 | 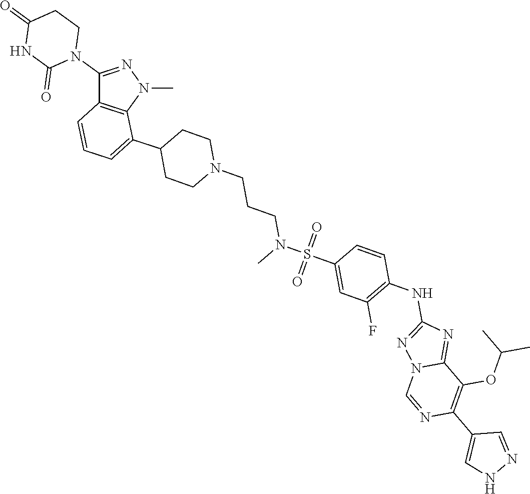 [SMILES: CC(C)Oc1c(-c2cn[nH]c2)ncn2nc(Nc3ccc(S(=O)(=O)N(C)CCCN4CCC(c5cccc6c(N7CCC(=O)NC7=O)nn(C)c56)CC4)cc3F)nc12] |
| 25 | 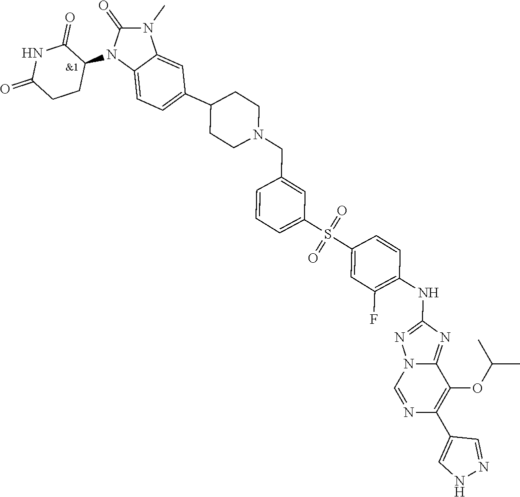 [SMILES: CC(C)Oc1c(-c2cn[nH]c2)ncn2nc(Nc3ccc(S(=O)(=O)c4cccc(CN5CCC(c6ccc7c(c6)n(C)c(=O)n7[C@H]6CCC(=O)NC6=O)CC5)c4)cc3F)nc12] |
| 26 | 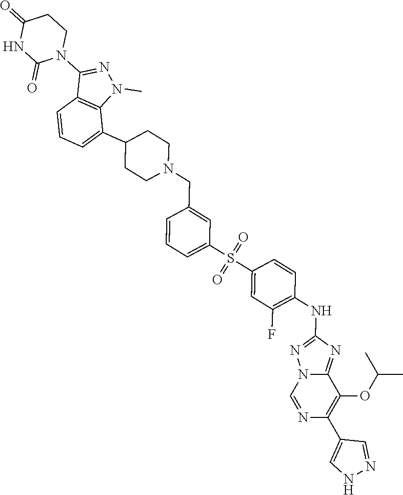 [SMILES: CC(C)Oc1c(-c2cn[nH]c2)ncn2nc(Nc3ccc(S(=O)(=O)c4cccc(CN5CCC(c6cccc7c(N8CCC(=O)NC8=O)nn(C)c67)CC5)c4)cc3F)nc12] |
| 27 | 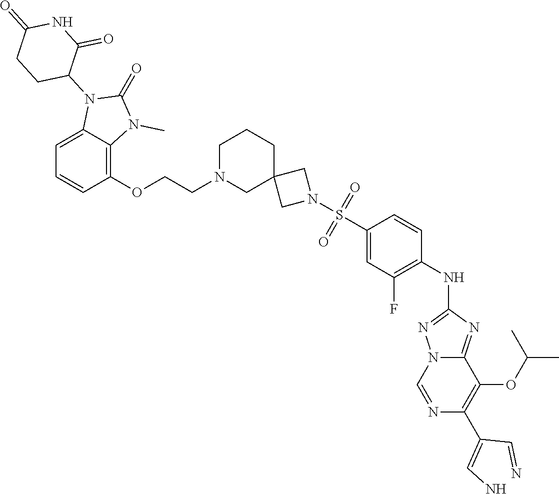 [SMILES: CC(C)Oc1c(/C(C=N)=C/N)ncn2nc(Nc3ccc(S(=O)(=O)N4CC5(CCCN(CCOc6cccc7c6n(C)c(=O)n7C6CCC(=O)NC6=O)C5)C4)cc3F)nc12] |
| 28 | 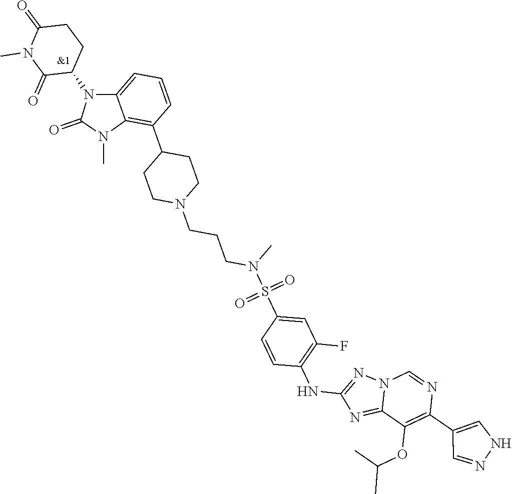 [SMILES: CC(C)Oc1c(-c2cn[nH]c2)ncn2nc(Nc3ccc(S(=O)(=O)N(C)CCCN4CCC(c5cccc6c5n(C)c(=O)n6[C@H]5CCC(=O)N(C)C5=O)CC4)cc3F)nc12] |
| 29 | 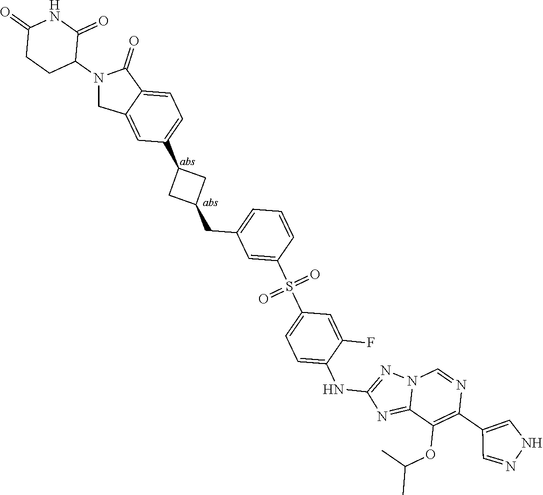 [SMILES: CC(C)Oc1c(-c2cn[nH]c2)ncn2nc(Nc3ccc(S(=O)(=O)c4cccc(C[C@H]5C[C@@H](c6ccc7c(c6)CN(C6CCC(=O)NC6=O)C7=O)C5)c4)cc3F)nc12] |
| 30 | 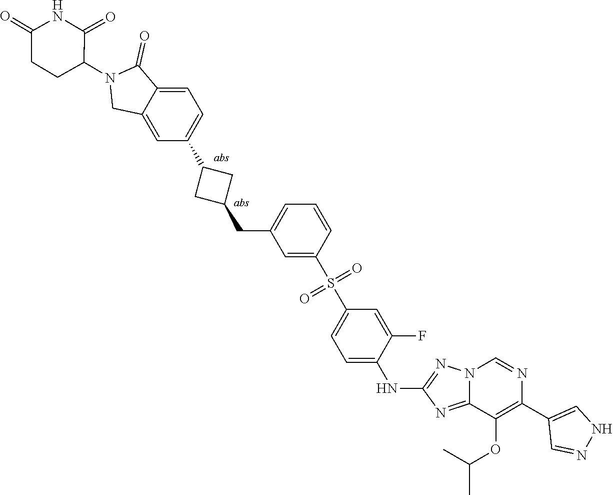 |
| 31 | 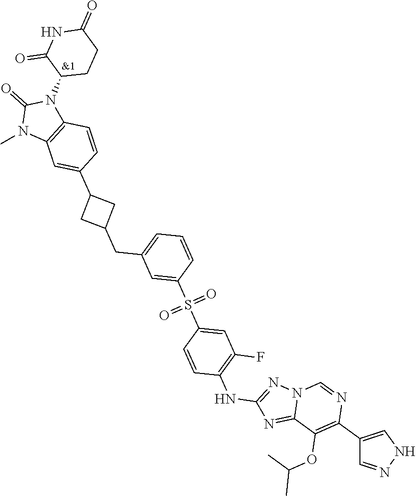 [SMILES: CC(C)Oc1c(-c2cn[nH]c2)ncn2nc(Nc3ccc(S(=O)(=O)c4cccc(CC5CC(c6ccc7c(c6)n(C)c(=O)n7[C@H]6CCC(=O)NC6=O)C5)c4)cc3F)nc12] |
| 32 | 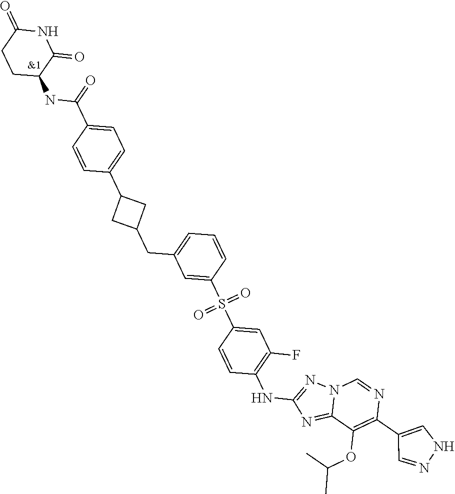 [SMILES: CC(C)Oc1c(-c2cn[nH]c2)ncn2nc(Nc3ccc(S(=O)(=O)c4cccc(CC5CC(c6ccc(C(=O)N[C@H]7CCC(=O)NC7=O)cc6)C5)c4)cc3F)nc12] |
| 33 | 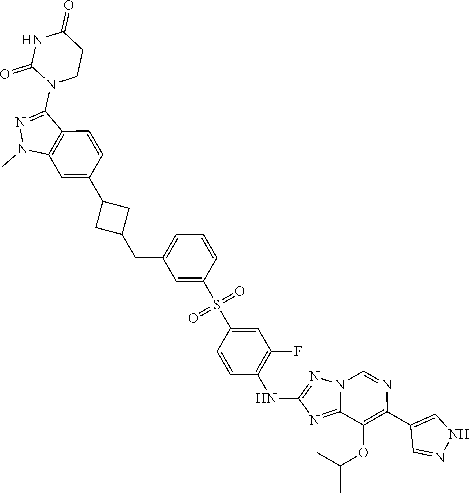 [SMILES: CC(C)Oc1c(-c2cn[nH]c2)ncn2nc(Nc3ccc(S(=O)(=O)c4cccc(CC5CC(c6ccc7c(N8CCC(=O)NC8=O)nn(C)c7c6)C5)c4)cc3F)nc12] |
| 34 | 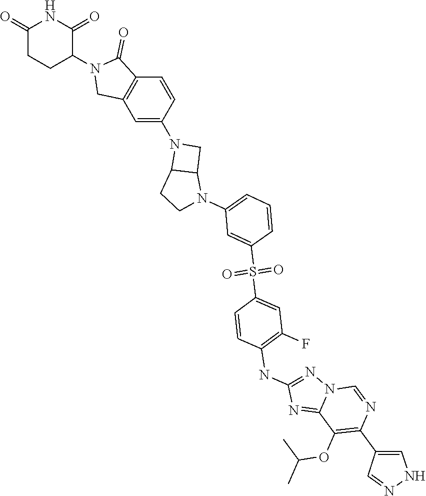 [SMILES: CC(C)Oc1c(-c2cn[nH]c2)ncn2nc(Nc3ccc(S(=O)(=O)c4cccc(N5CCC6C5CN6c5ccc6c(c5)CN(C5CCC(=O)NC5=O)C6=O)c4)cc3F)nc12] |
| 35 | 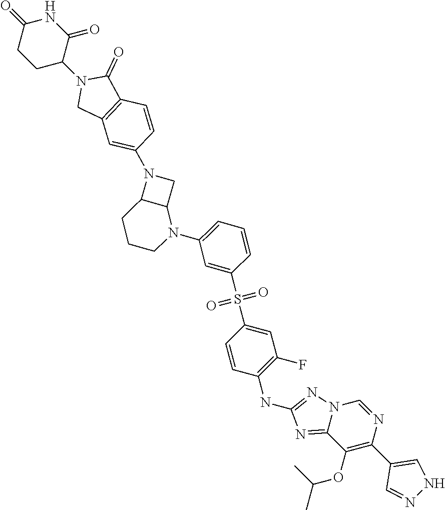 [SMILES: CC(C)Oc1c(-c2cn[nH]c2)ncn2nc(Nc3ccc(S(=O)(=O)c4cccc(N5CCCC6C5CN6c5ccc6c(c5)CN(C5CCC(=O)NC5=O)C6=O)c4)cc3F)nc12] |
| 36 | 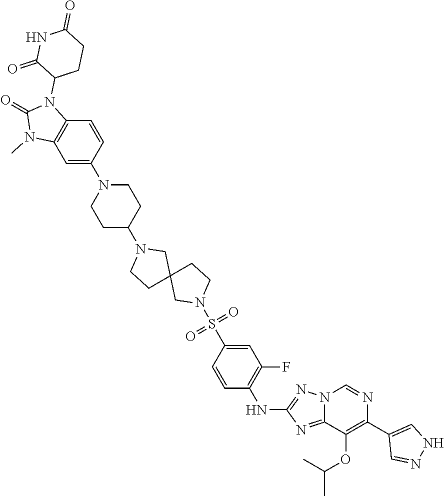 [SMILES: CC(C)Oc1c(-c2cn[nH]c2)ncn2nc(Nc3ccc(S(=O)(=O)N4CCC5(CCN(C6CCN(c7ccc8c(c7)n(C)c(=O)n8C7CCC(=O)NC7=O)CC6)C5)C4)cc3F)nc12] |
| 37 | 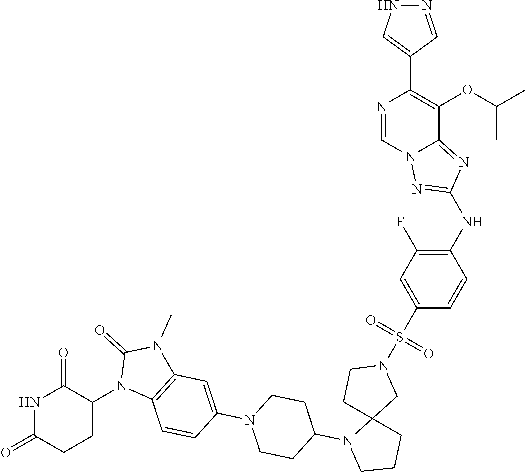 [SMILES: CC(C)Oc1c(-c2cn[nH]c2)ncn2nc(Nc3ccc(S(=O)(=O)N4CCC5(CCCN5C5CCN(c6ccc7c(c6)n(C)c(=O)n7C6CCC(=O)NC6=O)CC5)C4)cc3F)nc12] |
| 38 | 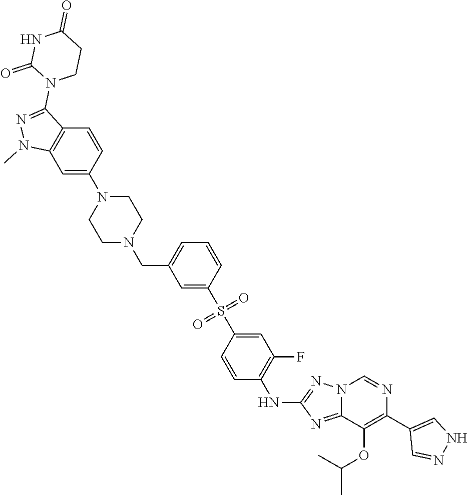 [SMILES: CC(C)Oc1c(-c2cn[nH]c2)ncn2nc(Nc3ccc(S(=O)(=O)c4cccc(CN5CCN(c6ccc7c(N8CCC(=O)NC8=O)nn(C)c7c6)CC5)c4)cc3F)nc12] |
| 39 | 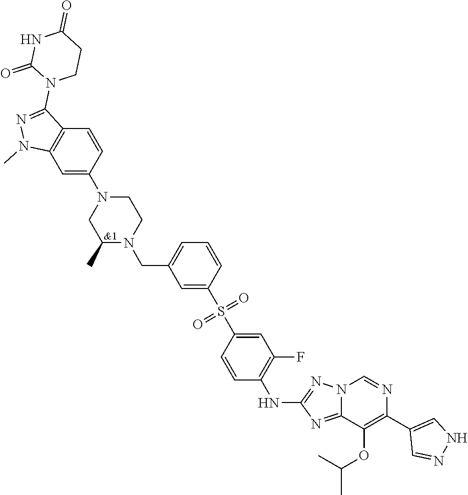 [SMILES: CC(C)Oc1c(-c2cn[nH]c2)ncn2nc(Nc3ccc(S(=O)(=O)c4cccc(CN5CCN(c6ccc7c(N8CCC(=O)NC8=O)nn(C)c7c6)C[C@@H]5C)c4)cc3F)nc12] |
| 40 | 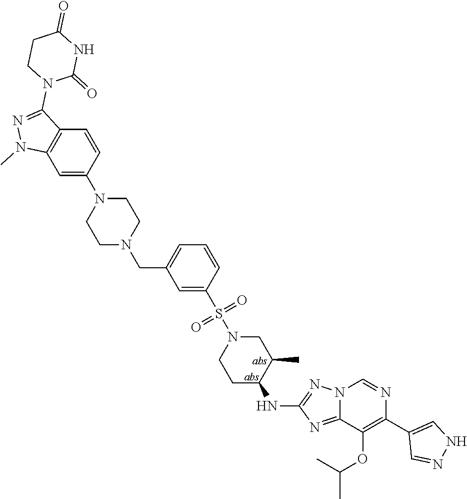 [SMILES: CC(C)Oc1c(-c2cn[nH]c2)ncn2nc(N[C@H]3CCN(S(=O)(=O)c4cccc(CN5CCN(c6ccc7c(N8CCC(=O)NC8=O)nn(C)c7c6)CC5)c4)C[C@H]3C)nc12] |
| 41 | 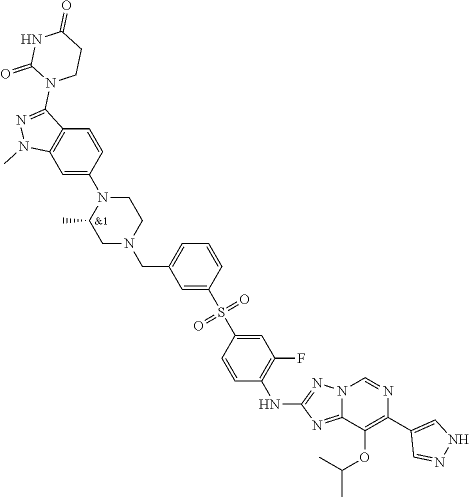 [SMILES: CC(C)Oc1c(-c2cn[nH]c2)ncn2nc(Nc3ccc(S(=O)(=O)c4cccc(CN5CCN(c6ccc7c(N8CCC(=O)NC8=O)nn(C)c7c6)[C@@H](C)C5)c4)cc3F)nc12] |
| 42 | 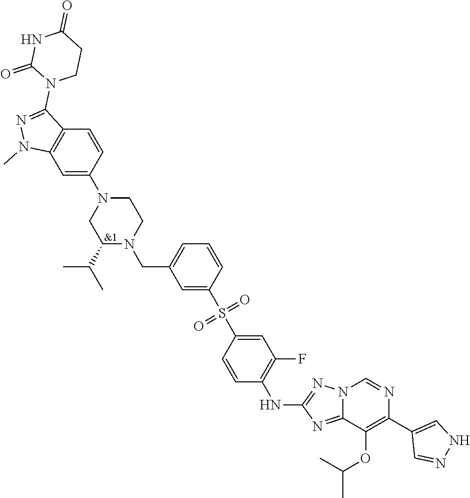 [SMILES: CC(C)Oc1c(-c2cn[nH]c2)ncn2nc(Nc3ccc(S(=O)(=O)c4cccc(CN5CCN(c6ccc7c(N8CCC(=O)NC8=O)nn(C)c7c6)C[C@H]5C(C)C)c4)cc3F)nc12] |
| 43 | 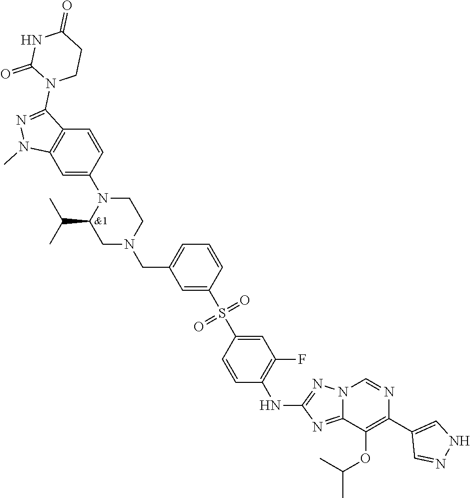 [SMILES: CC(C)Oc1c(-c2cn[nH]c2)ncn2nc(Nc3ccc(S(=O)(=O)c4cccc(CN5CCN(c6ccc7c(N8CCC(=O)NC8=O)nn(C)c7c6)[C@H](C(C)C)C5)c4)cc3F)nc12] |
| 44 | 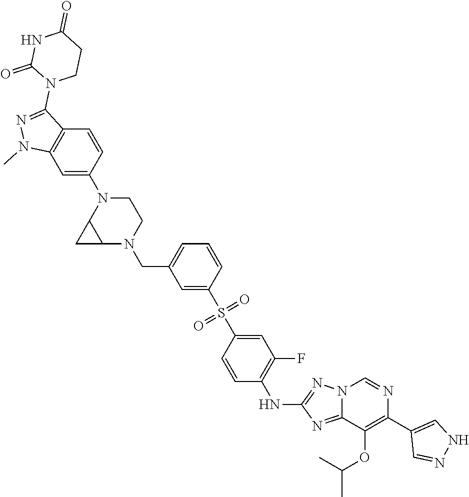 [SMILES: CC(C)Oc1c(-c2cn[nH]c2)ncn2nc(Nc3ccc(S(=O)(=O)c4cccc(CN5CCN(c6ccc7c(N8CCC(=O)NC8=O)nn(C)c7c6)C6CC65)c4)cc3F)nc12] |
| 45 | 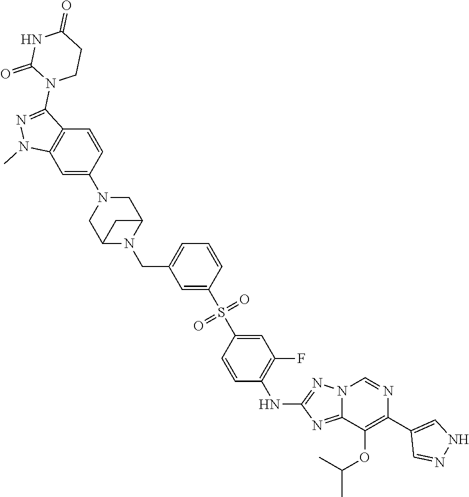 [SMILES: CC(C)Oc1c(-c2cn[nH]c2)ncn2nc(Nc3ccc(S(=O)(=O)c4cccc(CN5C6CC5CN(c5ccc7c(N8CCC(=O)NC8=O)nn(C)c7c5)C6)c4)cc3F)nc12] |
| 46 | 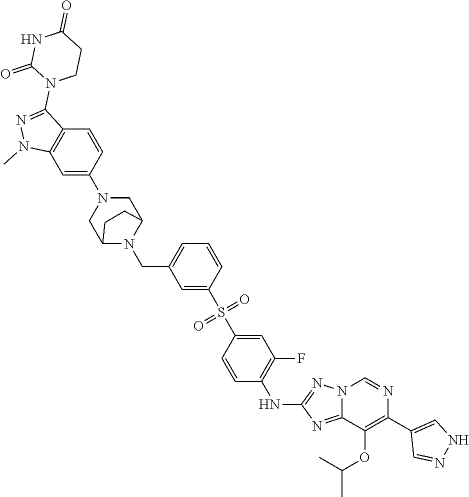 [SMILES: CC(C)Oc1c(-c2cn[nH]c2)ncn2nc(Nc3ccc(S(=O)(=O)c4cccc(CN5C6CCC5CN(c5ccc7c(N8CCC(=O)NC8=O)nn(C)c7c5)C6)c4)cc3F)nc12] |
| 47 | 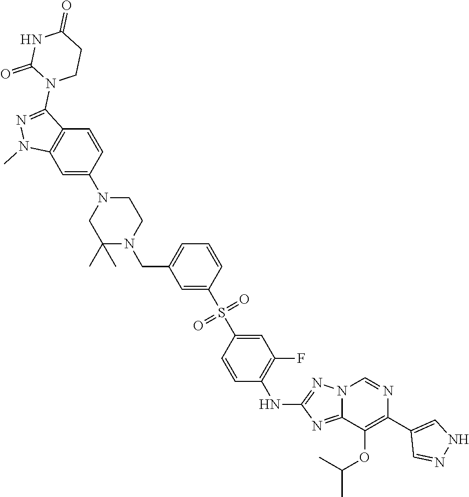 [SMILES: CC(C)Oc1c(-c2cn[nH]c2)ncn2nc(Nc3ccc(S(=O)(=O)c4cccc(CN5CCN(c6ccc7c(N8CCC(=O)NC8=O)nn(C)c7c6)CC5(C)C)c4)cc3F)nc12] |
| 48 | 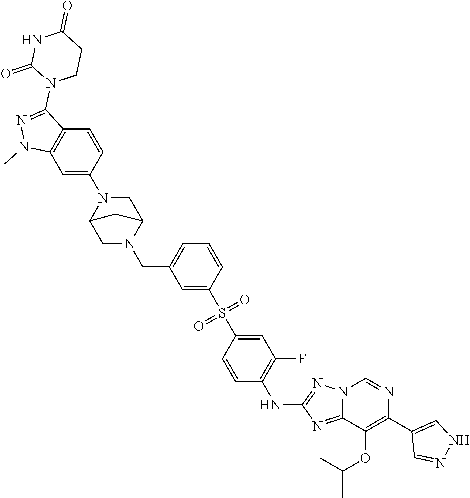 [SMILES: CC(C)Oc1c(-c2cn[nH]c2)ncn2nc(Nc3ccc(S(=O)(=O)c4cccc(CN5CC6CC5CN6c5ccc6c(N7CCC(=O)NC7=O)nn(C)c6c5)c4)cc3F)nc12] |
| 49 | 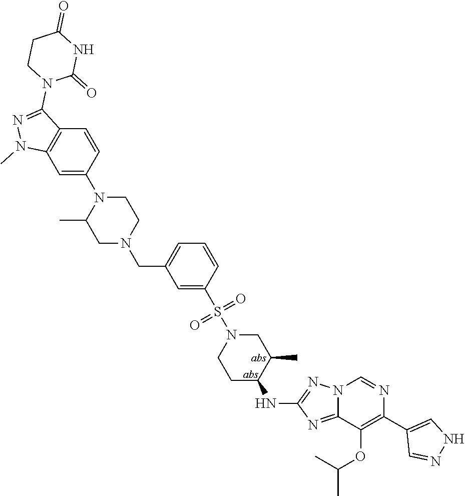 [SMILES: CC(C)Oc1c(-c2cn[nH]c2)ncn2nc(N[C@H]3CCN(S(=O)(=O)c4cccc(CN5CCN(c6ccc7c(N8CCC(=O)NC8=O)nn(C)c7c6)C(C)C5)c4)C[C@H]3C)nc12] |
| 50 | 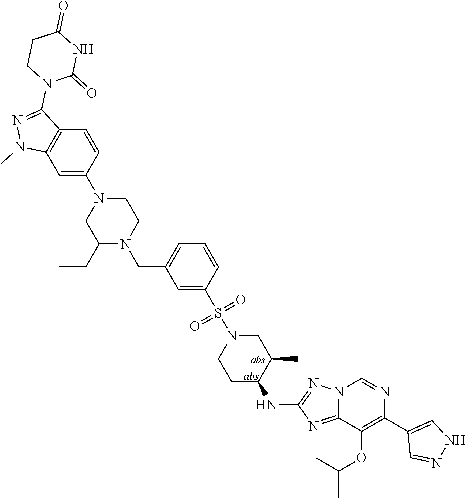 [SMILES: CCC1CN(c2ccc3c(N4CCC(=O)NC4=O)nn(C)c3c2)CCN1Cc1cccc(S(=O)(=O)N2CC[C@H](Nc3nc4c(OC(C)C)c(-c5cn[nH]c5)ncn4n3)[C@H](C)C2)c1] |
| 51 | 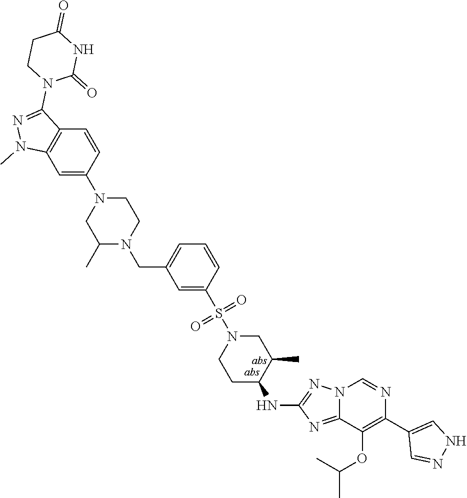 [SMILES: CC(C)Oc1c(-c2cn[nH]c2)ncn2nc(N[C@H]3CCN(S(=O)(=O)c4cccc(CN5CCN(c6ccc7c(N8CCC(=O)NC8=O)nn(C)c7c6)CC5C)c4)C[C@H]3C)nc12] |
| 52 | 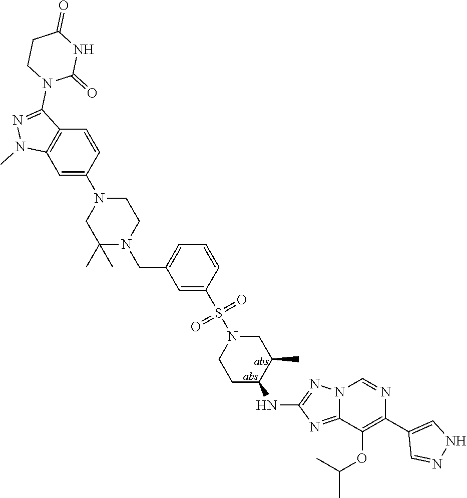 [SMILES: CC(C)Oc1c(-c2cn[nH]c2)ncn2nc(N[C@H]3CCN(S(=O)(=O)c4cccc(CN5CCN(c6ccc7c(N8CCC(=O)NC8=O)nn(C)c7c6)CC5(C)C)c4)C[C@H]3C)nc12] |
| 53 | 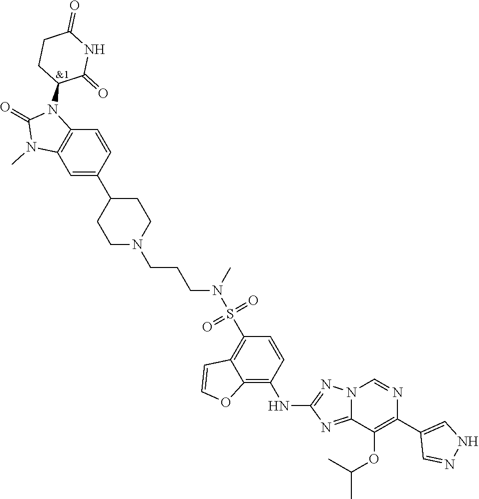 [SMILES: CC(C)Oc1c(-c2cn[nH]c2)ncn2nc(Nc3ccc(S(=O)(=O)N(C)CCCN4CCC(c5ccc6c(c5)n(C)c(=O)n6[C@H]5CCC(=O)NC5=O)CC4)c4ccoc34)nc12] |
| 54 | 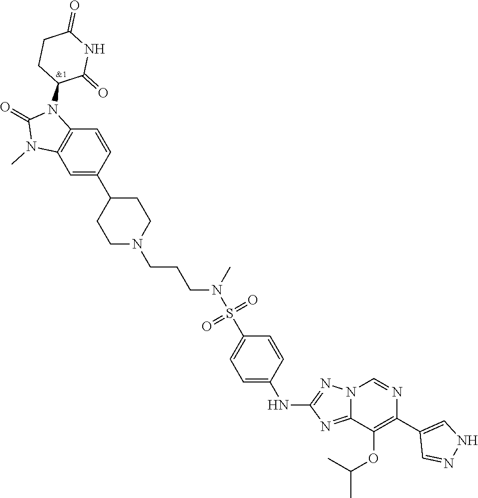 [SMILES: CC(C)Oc1c(-c2cn[nH]c2)ncn2nc(Nc3ccc(S(=O)(=O)N(C)CCCN4CCC(c5ccc6c(c5)n(C)c(=O)n6[C@H]5CCC(=O)NC5=O)CC4)cc3)nc12] |
| 55 | 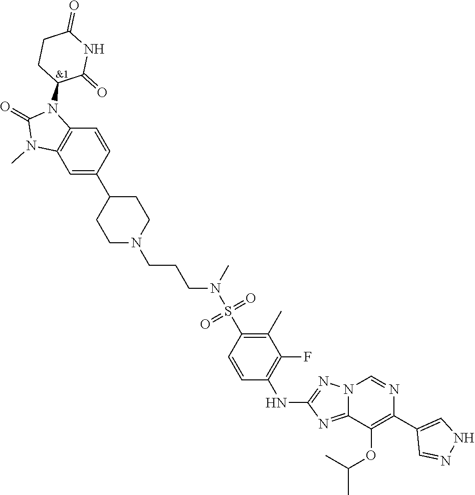 [SMILES: Cc1c(S(=O)(=O)N(C)CCCN2CCC(c3ccc4c(c3)n(C)c(=O)n4[C@H]3CCC(=O)NC3=O)CC2)ccc(Nc2nc3c(OC(C)C)c(-c4cn[nH]c4)ncn3n2)c1F] |
| 56 | 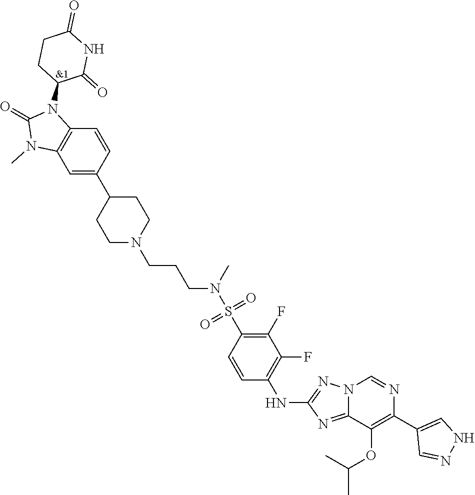 [SMILES: CC(C)Oc1c(-c2cn[nH]c2)ncn2nc(Nc3ccc(S(=O)(=O)N(C)CCCN4CCC(c5ccc6c(c5)n(C)c(=O)n6[C@H]5CCC(=O)NC5=O)CC4)c(F)c3F)nc12] |
| 57 | 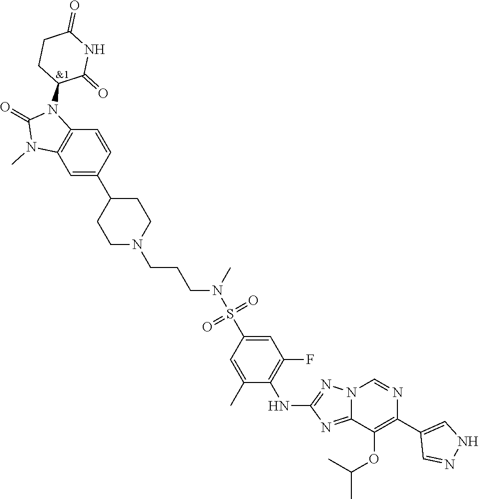 [SMILES: Cc1cc(S(=O)(=O)N(C)CCCN2CCC(c3ccc4c(c3)n(C)c(=O)n4[C@H]3CCC(=O)NC3=O)CC2)cc(F)c1Nc1nc2c(OC(C)C)c(-c3cn[nH]c3)ncn2n1] |
| 58 | 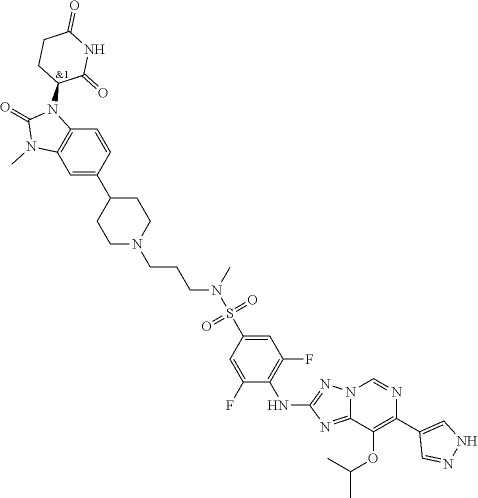 [SMILES: CC(C)Oc1c(-c2cn[nH]c2)ncn2nc(Nc3c(F)cc(S(=O)(=O)N(C)CCCN4CCC(c5ccc6c(c5)n(C)c(=O)n6[C@H]5CCC(=O)NC5=O)CC4)cc3F)nc12] |
| 59 | 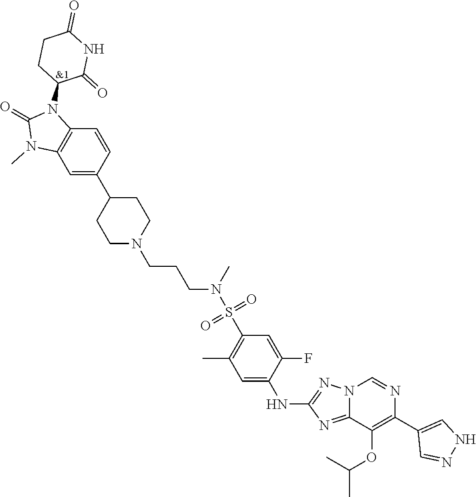 [SMILES: Cc1cc(Nc2nc3c(OC(C)C)c(-c4cn[nH]c4)ncn3n2)c(F)cc1S(=O)(=O)N(C)CCCN1CCC(c2ccc3c(c2)n(C)c(=O)n3[C@H]2CCC(=O)NC2=O)CC1] |
| 60 | 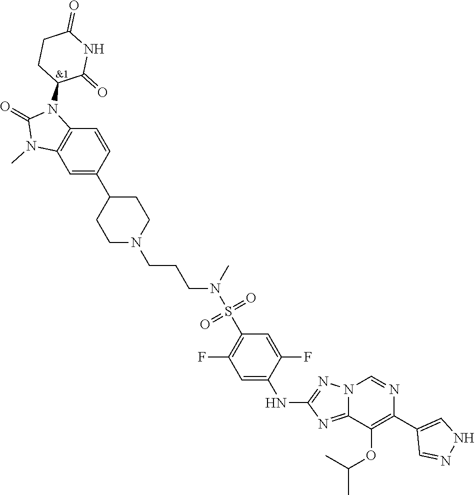 [SMILES: CC(C)Oc1c(-c2cn[nH]c2)ncn2nc(Nc3cc(F)c(S(=O)(=O)N(C)CCCN4CCC(c5ccc6c(c5)n(C)c(=O)n6[C@H]5CCC(=O)NC5=O)CC4)cc3F)nc12] |
| 61 | 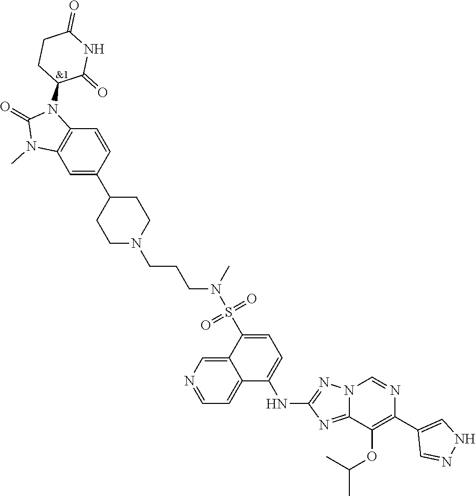 [SMILES: CC(C)Oc1c(-c2cn[nH]c2)ncn2nc(Nc3ccc(S(=O)(=O)N(C)CCCN4CCC(c5ccc6c(c5)n(C)c(=O)n6[C@H]5CCC(=O)NC5=O)CC4)c4cnccc34)nc12] |
| 62 | 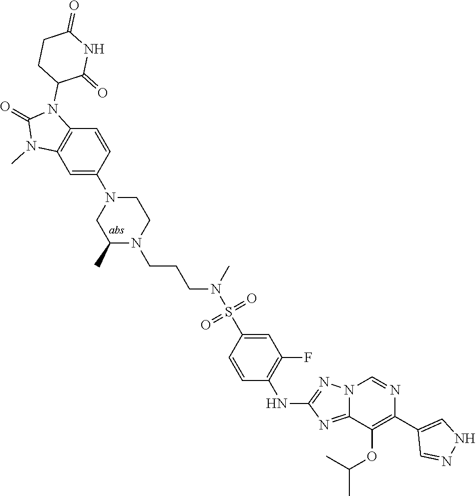 [SMILES: CC(C)Oc1c(-c2cn[nH]c2)ncn2nc(Nc3ccc(S(=O)(=O)N(C)CCCN4CCN(c5ccc6c(c5)n(C)c(=O)n6C5CCC(=O)NC5=O)C[C@@H]4C)cc3F)nc12] |
| 63 | 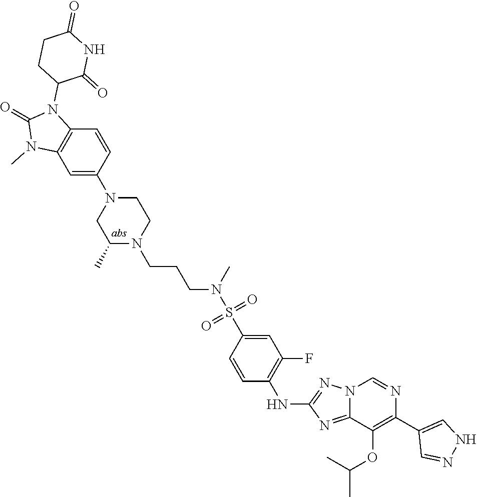 [SMILES: CC(C)Oc1c(-c2cn[nH]c2)ncn2nc(Nc3ccc(S(=O)(=O)N(C)CCCN4CCN(c5ccc6c(c5)n(C)c(=O)n6C5CCC(=O)NC5=O)C[C@H]4C)cc3F)nc12] |
| 64 | 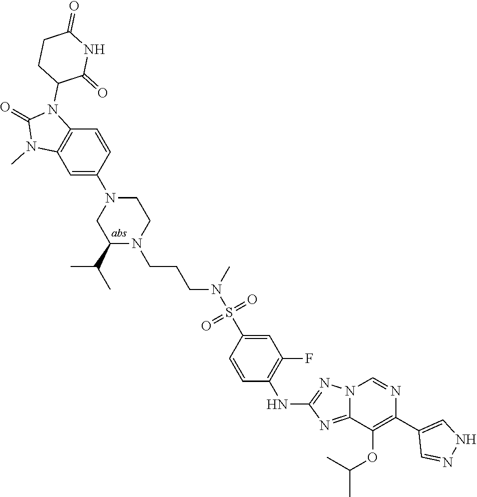 [SMILES: CC(C)Oc1c(-c2cn[nH]c2)ncn2nc(Nc3ccc(S(=O)(=O)N(C)CCCN4CCN(c5ccc6c(c5)n(C)c(=O)n6C5CCC(=O)NC5=O)C[C@@H]4C(C)C)cc3F)nc12] |
| 65 | 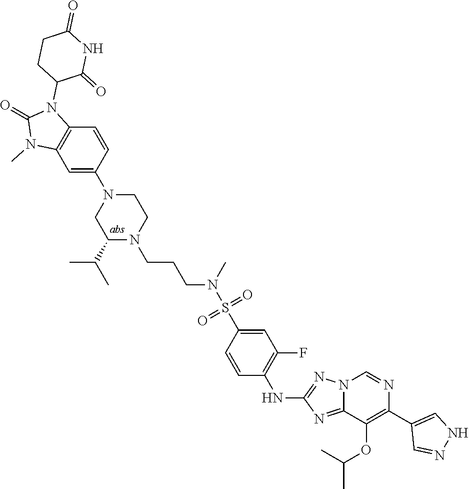 [SMILES: CC(C)Oc1c(-c2cn[nH]c2)ncn2nc(Nc3ccc(S(=O)(=O)N(C)CCCN4CCN(c5ccc6c(c5)n(C)c(=O)n6C5CCC(=O)NC5=O)C[C@H]4C(C)C)cc3F)nc12] |
| 66 | 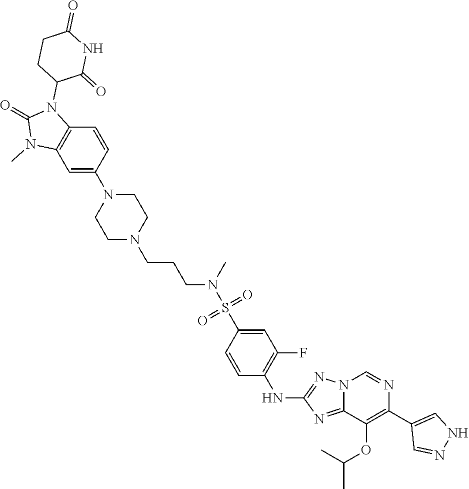 [SMILES: CC(C)Oc1c(-c2cn[nH]c2)ncn2nc(Nc3ccc(S(=O)(=O)N(C)CCCN4CCN(c5ccc6c(c5)n(C)c(=O)n6C5CCC(=O)NC5=O)CC4)cc3F)nc12] |
| 67 | 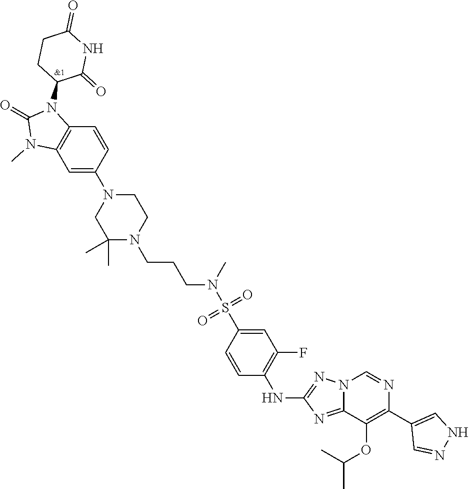 [SMILES: CC(C)Oc1c(-c2cn[nH]c2)ncn2nc(Nc3ccc(S(=O)(=O)N(C)CCCN4CCN(c5ccc6c(c5)n(C)c(=O)n6[C@H]5CCC(=O)NC5=O)CC4(C)C)cc3F)nc12] |
| 68 | 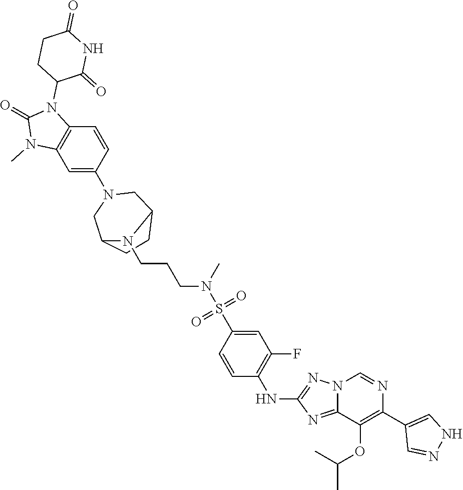 [SMILES: CC(C)Oc1c(-c2cn[nH]c2)ncn2nc(Nc3ccc(S(=O)(=O)N(C)CCCN4C5CCC4CN(c4ccc6c(c4)n(C)c(=O)n6C4CCC(=O)NC4=O)C5)cc3F)nc12] |
| 69 | 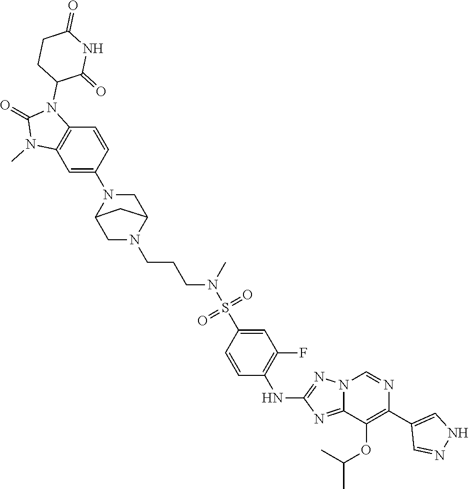 [SMILES: CC(C)Oc1c(-c2cn[nH]c2)ncn2nc(Nc3ccc(S(=O)(=O)N(C)CCCN4CC5CC4CN5c4ccc5c(c4)n(C)c(=O)n5C4CCC(=O)NC4=O)cc3F)nc12] |
| 70 | 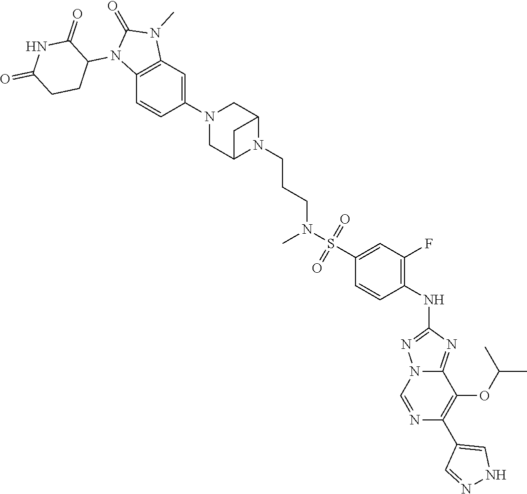 [SMILES: CC(C)Oc1c(-c2cn[nH]c2)ncn2nc(Nc3ccc(S(=O)(=O)N(C)CCCN4C5CC4CN(c4ccc6c(c4)n(C)c(=O)n6C4CCC(=O)NC4=O)C5)cc3F)nc12] |
| 71 | 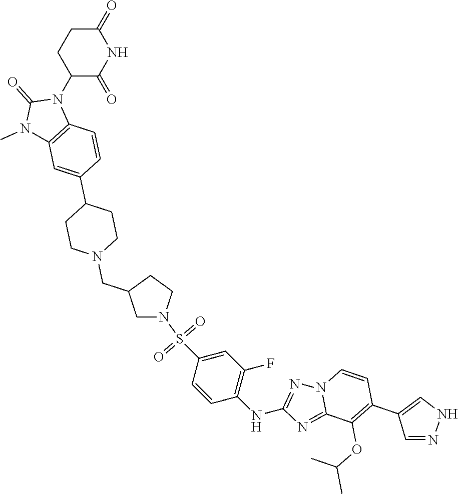 [SMILES: CC(C)Oc1c(-c2cn[nH]c2)ccn2nc(Nc3ccc(S(=O)(=O)N4CCC(CN5CCC(c6ccc7c(c6)n(C)c(=O)n7C6CCC(=O)NC6=O)CC5)C4)cc3F)nc12] |
| 72 | 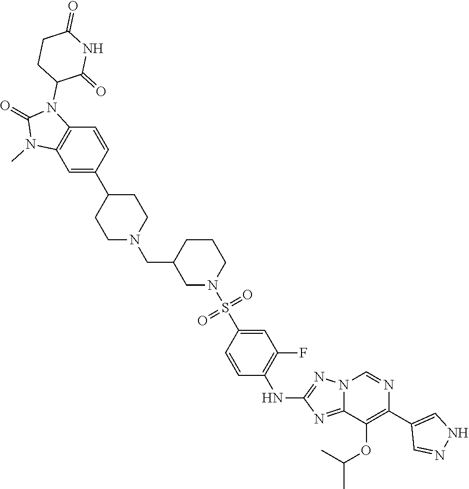 [SMILES: CC(C)Oc1c(-c2cn[nH]c2)ncn2nc(Nc3ccc(S(=O)(=O)N4CCCC(CN5CCC(c6ccc7c(c6)n(C)c(=O)n7C6CCC(=O)NC6=O)CC5)C4)cc3F)nc12] |
| 73 | 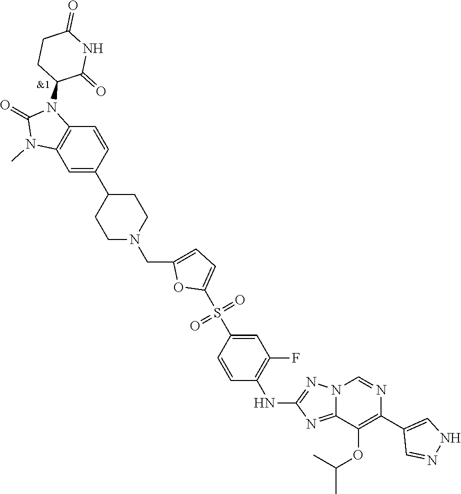 [SMILES: CC(C)Oc1c(-c2cn[nH]c2)ncn2nc(Nc3ccc(S(=O)(=O)c4ccc(CN5CCC(c6ccc7c(c6)n(C)c(=O)n7[C@H]6CCC(=O)NC6=O)CC5)o4)cc3F)nc12] |
| 74 | 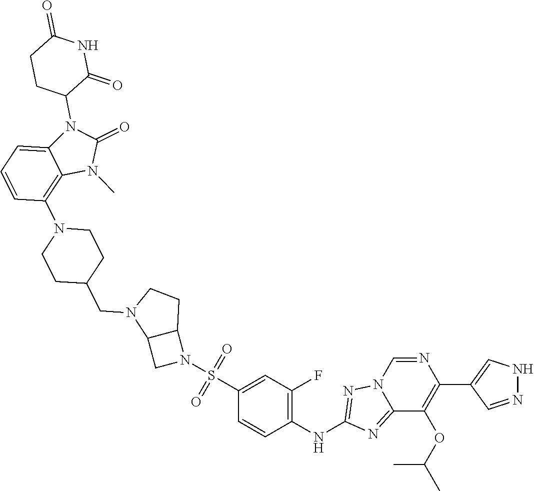 [SMILES: CC(C)Oc1c(-c2cn[nH]c2)ncn2nc(Nc3ccc(S(=O)(=O)N4CC5C4CCN5CC4CCN(c5cccc6c5n(C)c(=O)n6C5CCC(=O)NC5=O)CC4)cc3F)nc12] |
| 75 | 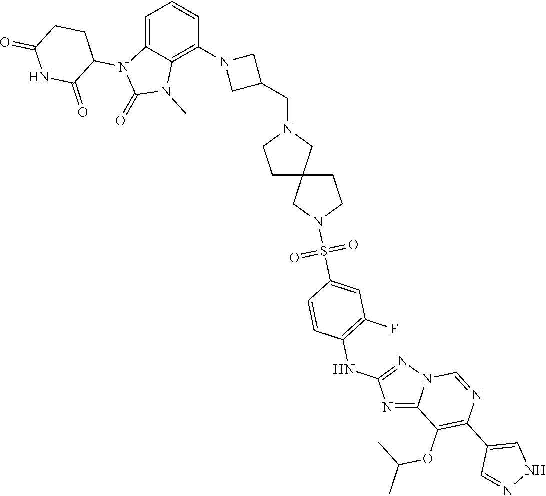 [SMILES: CC(C)Oc1c(-c2cn[nH]c2)ncn2nc(Nc3ccc(S(=O)(=O)N4CCC5(CCN(CC6CN(c7cccc8c7n(C)c(=O)n8C7CCC(=O)NC7=O)C6)C5)C4)cc3F)nc12] |
| 76 | 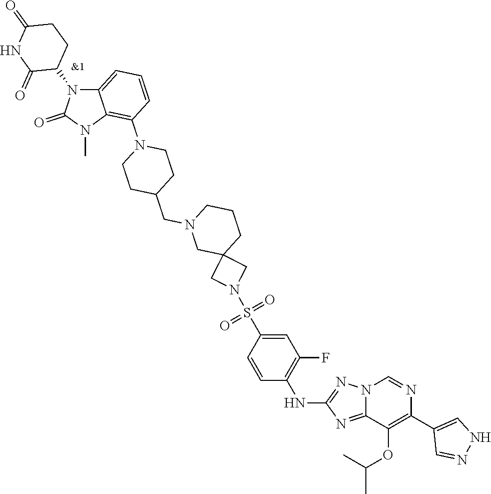 |
| 77 | 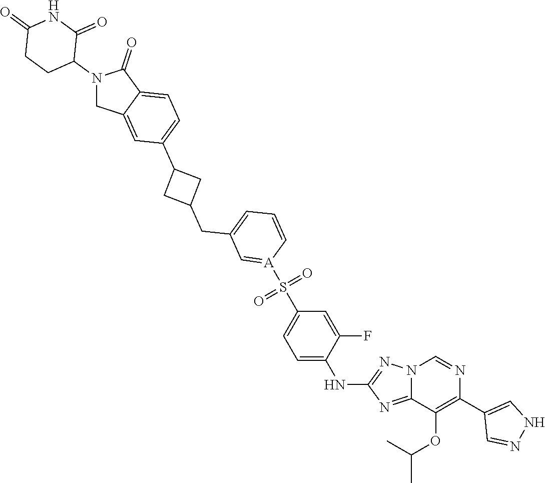 |
| 78 | 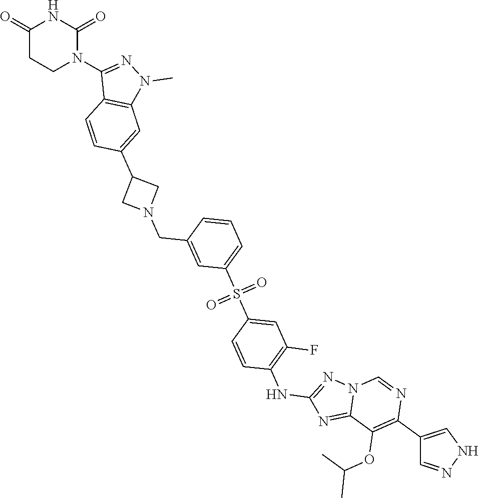 [SMILES: CC(C)Oc1c(-c2cn[nH]c2)ncn2nc(Nc3ccc(S(=O)(=O)c4cccc(CN5CC(c6ccc7c(N8CCC(=O)NC8=O)nn(C)c7c6)C5)c4)cc3F)nc12] |
| 79 | 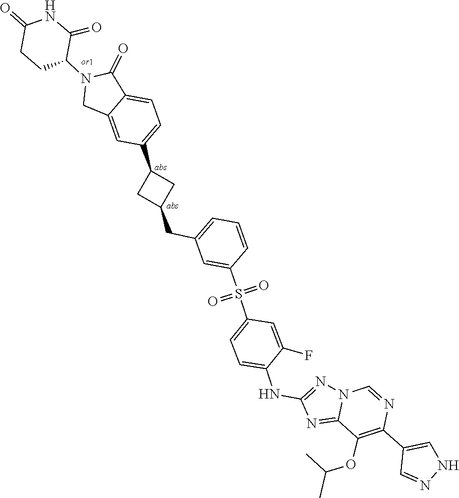 [SMILES: CC(C)Oc1c(-c2cn[nH]c2)ncn2nc(Nc3ccc(S(=O)(=O)c4cccc(C[C@H]5C[C@@H](c6ccc7c(c6)CN([C@@H]6CCC(=O)NC6=O)C7=O)C5)c4)cc3F)nc12] |
| 80 | 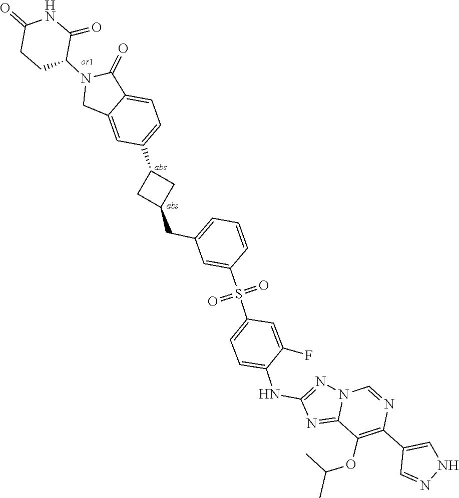 [SMILES: CC(C)Oc1c(-c2cn[nH]c2)ncn2nc(Nc3ccc(S(=O)(=O)c4cccc(C[C@H]5C[C@H](c6ccc7c(c6)CN([C@@H]6CCC(=O)NC6=O)C7=O)C5)c4)cc3F)nc12] |
| 81 | 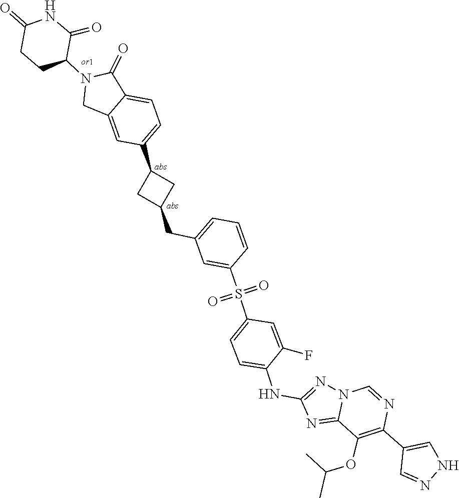 [SMILES: CC(C)Oc1c(-c2cn[nH]c2)ncn2nc(Nc3ccc(S(=O)(=O)c4cccc(C[C@H]5C[C@@H](c6ccc7c(c6)CN([C@H]6CCC(=O)NC6=O)C7=O)C5)c4)cc3F)nc12] |
| 82 | 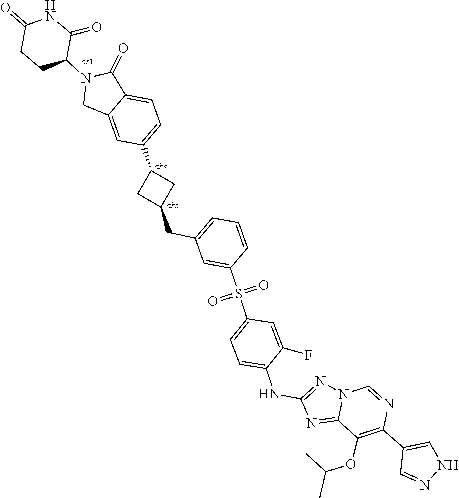 [SMILES: CC(C)Oc1c(-c2cn[nH]c2)ncn2nc(Nc3ccc(S(=O)(=O)c4cccc(C[C@H]5C[C@H](c6ccc7c(c6)CN([C@H]6CCC(=O)NC6=O)C7=O)C5)c4)cc3F)nc12] |
| 83 | 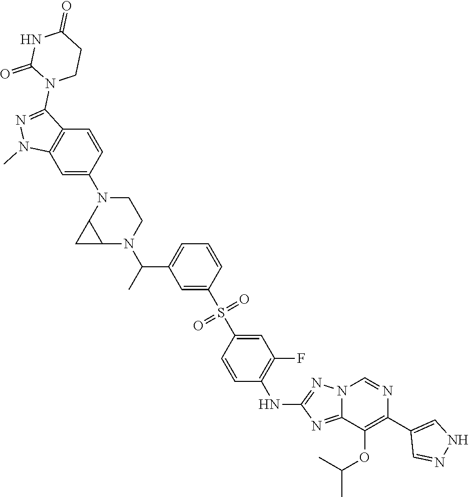 [SMILES: CC(C)Oc1c(-c2cn[nH]c2)ncn2nc(Nc3ccc(S(=O)(=O)c4cccc(C(C)N5CCN(c6ccc7c(N8CCC(=O)NC8=O)nn(C)c7c6)C6CC65)c4)cc3F)nc12] |
| 84 | 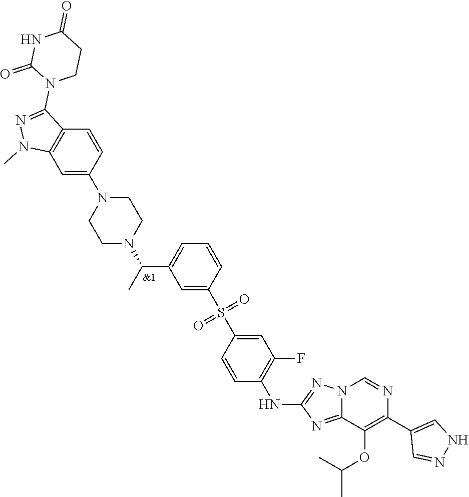 [SMILES: CC(C)Oc1c(-c2cn[nH]c2)ncn2nc(Nc3ccc(S(=O)(=O)c4cccc([C@H](C)N5CCN(c6ccc7c(N8CCC(=O)NC8=O)nn(C)c7c6)CC5)c4)cc3F)nc12] |
| 85 | 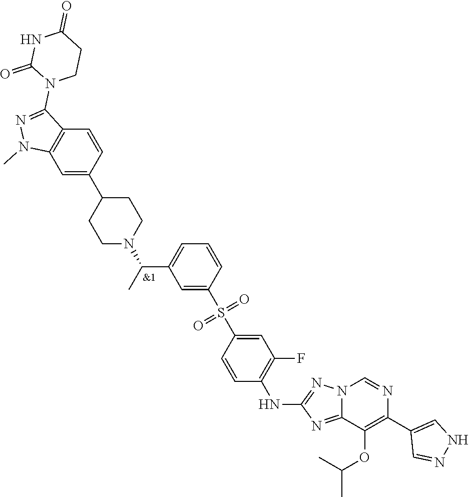 [SMILES: CC(C)Oc1c(-c2cn[nH]c2)ncn2nc(Nc3ccc(S(=O)(=O)c4cccc([C@H](C)N5CCC(c6ccc7c(N8CCC(=O)NC8=O)nn(C)c7c6)CC5)c4)cc3F)nc12] |
| 86 | 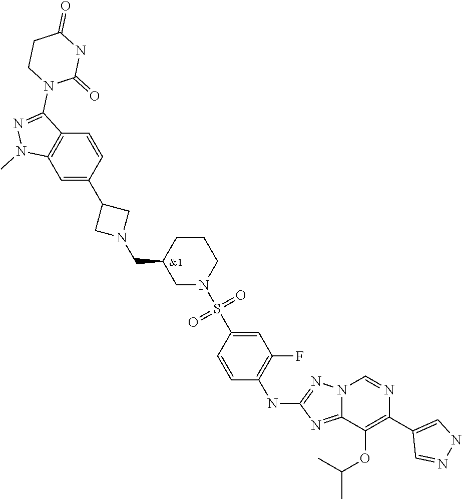 |
| 87 | 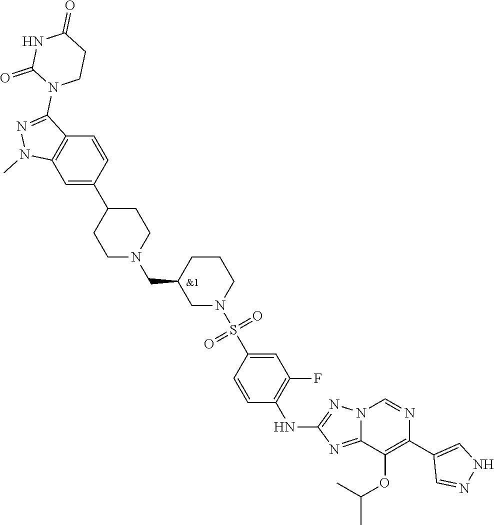 [SMILES: CC(C)Oc1c(-c2cn[nH]c2)ncn2nc(Nc3ccc(S(=O)(=O)N4CCC[C@H](CN5CCC(c6ccc7c(N8CCC(=O)NC8=O)nn(C)c7c6)CC5)C4)cc3F)nc12] |
| 88 | 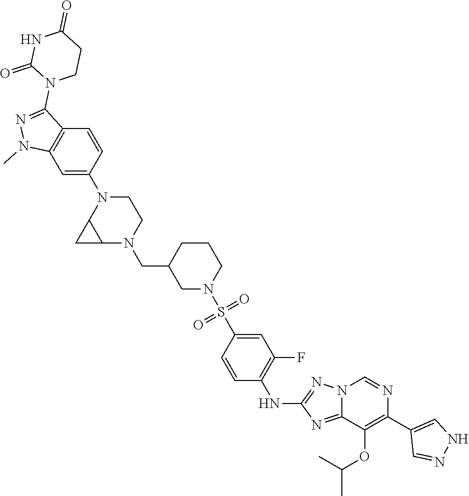 |
| 89 | 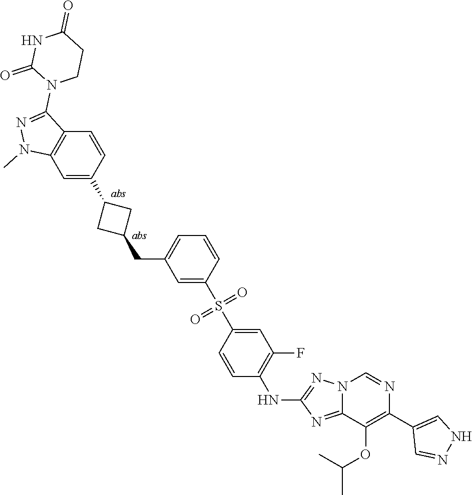 [SMILES: CC(C)Oc1c(-c2cn[nH]c2)ncn2nc(Nc3ccc(S(=O)(=O)c4cccc(C[C@H]5C[C@H](c6ccc7c(N8CCC(=O)NC8=O)nn(C)c7c6)C5)c4)cc3F)nc12] |
| 90 | 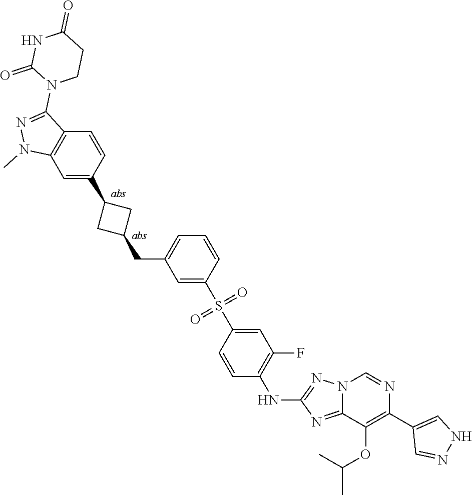 [SMILES: CC(C)Oc1c(-c2cn[nH]c2)ncn2nc(Nc3ccc(S(=O)(=O)c4cccc(C[C@H]5C[C@@H](c6ccc7c(N8CCC(=O)NC8=O)nn(C)c7c6)C5)c4)cc3F)nc12] |
| 91 | 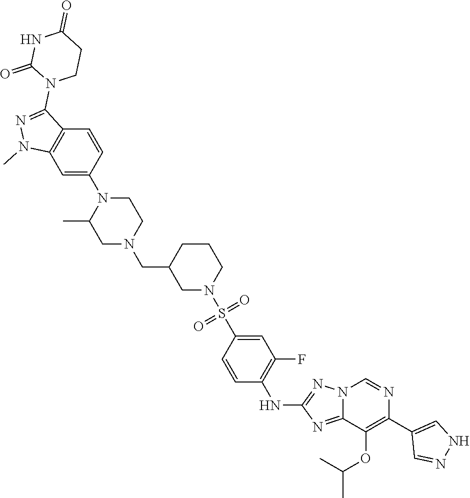 [SMILES: CC(C)Oc1c(-c2cn[nH]c2)ncn2nc(Nc3ccc(S(=O)(=O)N4CCCC(CN5CCN(c6ccc7c(N8CCC(=O)NC8=O)nn(C)c7c6)C(C)C5)C4)cc3F)nc12] |
| 92 | 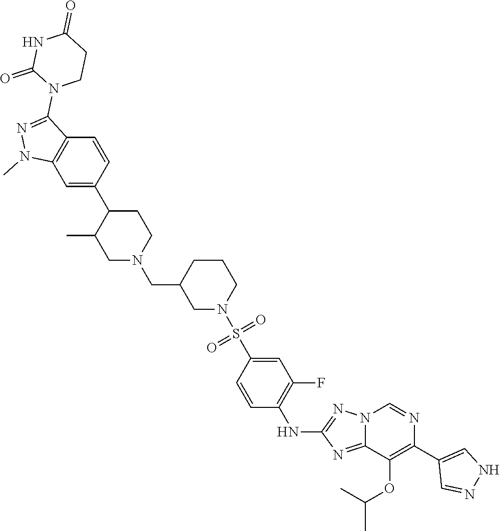 [SMILES: CC(C)Oc1c(-c2cn[nH]c2)ncn2nc(Nc3ccc(S(=O)(=O)N4CCCC(CN5CCC(c6ccc7c(N8CCC(=O)NC8=O)nn(C)c7c6)C(C)C5)C4)cc3F)nc12] |
| 93 | 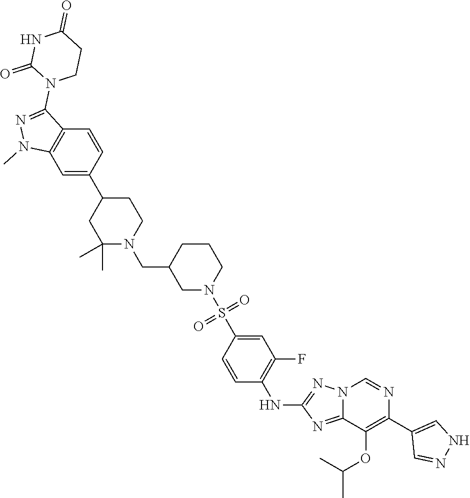 [SMILES: CC(C)Oc1c(-c2cn[nH]c2)ncn2nc(Nc3ccc(S(=O)(=O)N4CCCC(CN5CCC(c6ccc7c(N8CCC(=O)NC8=O)nn(C)c7c6)CC5(C)C)C4)cc3F)nc12] |
| 94 | 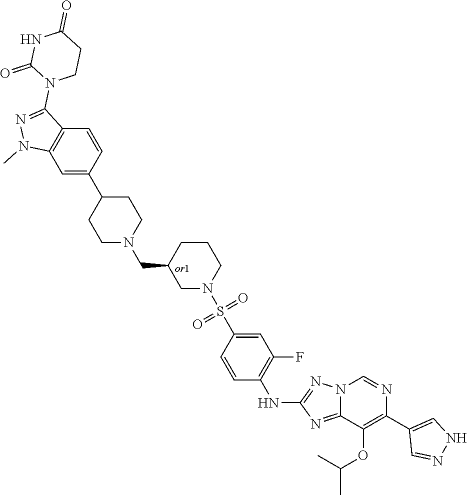 [SMILES: CC(C)Oc1c(-c2cn[nH]c2)ncn2nc(Nc3ccc(S(=O)(=O)N4CCC[C@H](CN5CCC(c6ccc7c(N8CCC(=O)NC8=O)nn(C)c7c6)CC5)C4)cc3F)nc12] |
| 95 | 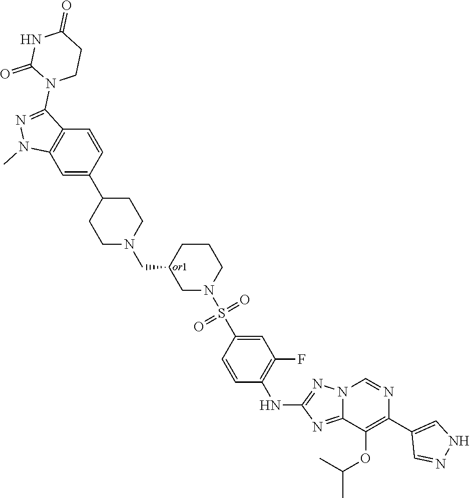 [SMILES: CC(C)Oc1c(-c2cn[nH]c2)ncn2nc(Nc3ccc(S(=O)(=O)N4CCC[C@@H](CN5CCC(c6ccc7c(N8CCC(=O)NC8=O)nn(C)c7c6)CC5)C4)cc3F)nc12] |
| 96 | 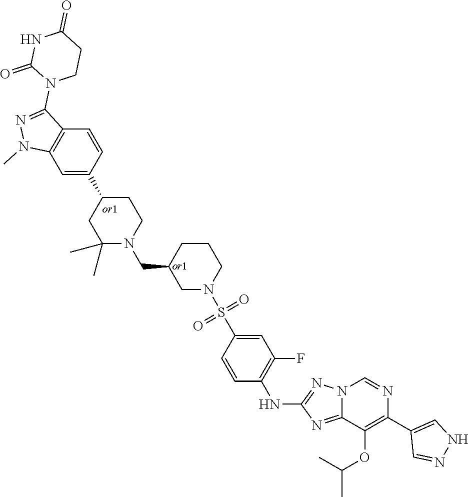 [SMILES: CC(C)Oc1c(-c2cn[nH]c2)ncn2nc(Nc3ccc(S(=O)(=O)N4CCC[C@H](CN5CC[C@@H](c6ccc7c(N8CCC(=O)NC8=O)nn(C)c7c6)CC5(C)C)C4)cc3F)nc12] |
| 97 | 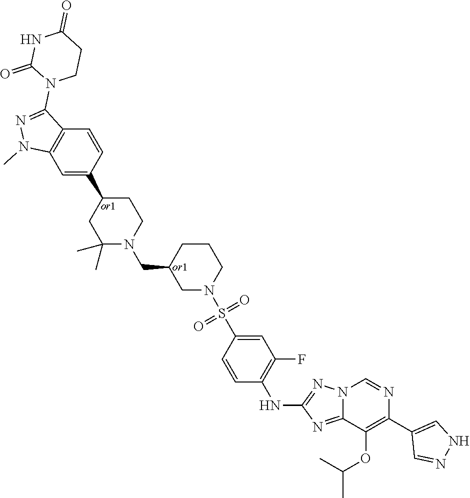 [SMILES: CC(C)Oc1c(-c2cn[nH]c2)ncn2nc(Nc3ccc(S(=O)(=O)N4CCC[C@H](CN5CC[C@H](c6ccc7c(N8CCC(=O)NC8=O)nn(C)c7c6)CC5(C)C)C4)cc3F)nc12] |
| 98 | 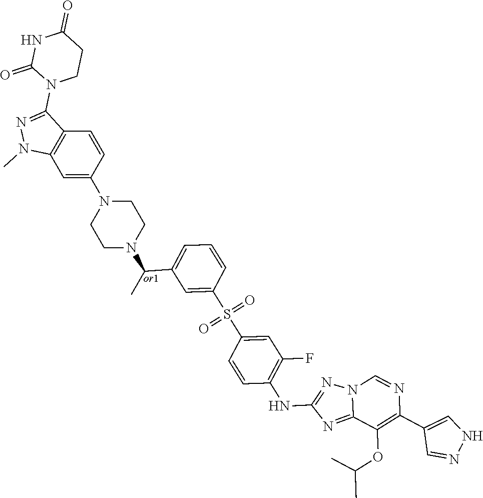 [SMILES: CC(C)Oc1c(-c2cn[nH]c2)ncn2nc(Nc3ccc(S(=O)(=O)c4cccc([C@@H](C)N5CCN(c6ccc7c(N8CCC(=O)NC8=O)nn(C)c7c6)CC5)c4)cc3F)nc12] |
| 99 | 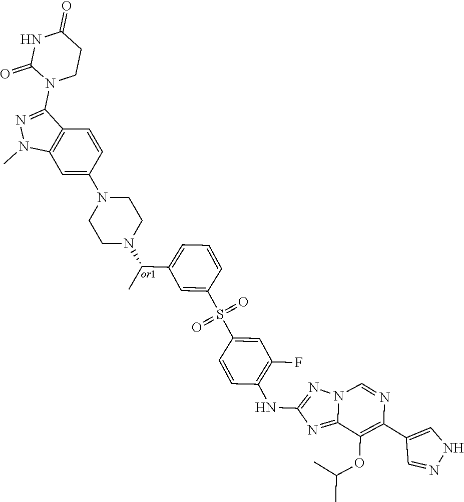 [SMILES: CC(C)Oc1c(-c2cn[nH]c2)ncn2nc(Nc3ccc(S(=O)(=O)c4cccc([C@H](C)N5CCN(c6ccc7c(N8CCC(=O)NC8=O)nn(C)c7c6)CC5)c4)cc3F)nc12] |
| 100 | 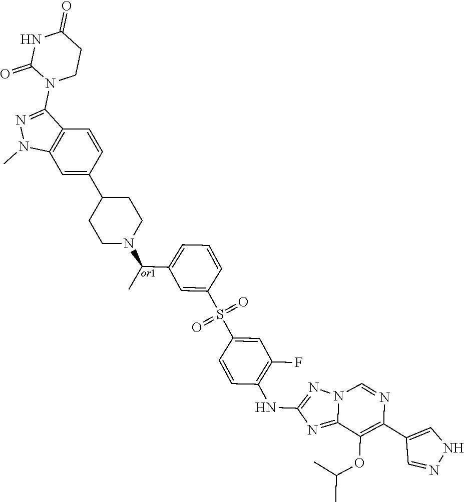 [SMILES: CC(C)Oc1c(-c2cn[nH]c2)ncn2nc(Nc3ccc(S(=O)(=O)c4cccc([C@@H](C)N5CCC(c6ccc7c(N8CCC(=O)NC8=O)nn(C)c7c6)CC5)c4)cc3F)nc12] |
| 101 | 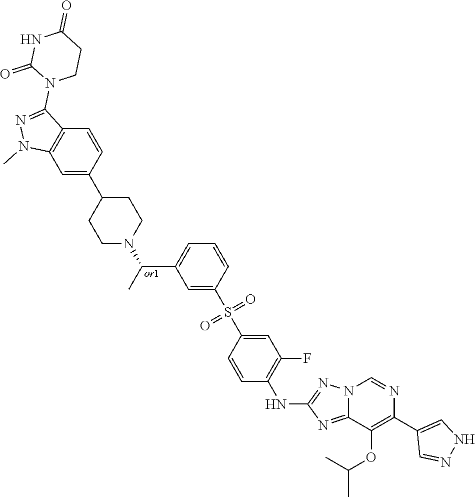 [SMILES: CC(C)Oc1c(-c2cn[nH]c2)ncn2nc(Nc3ccc(S(=O)(=O)c4cccc([C@H](C)N5CCC(c6ccc7c(N8CCC(=O)NC8=O)nn(C)c7c6)CC5)c4)cc3F)nc12] |
| 102 | 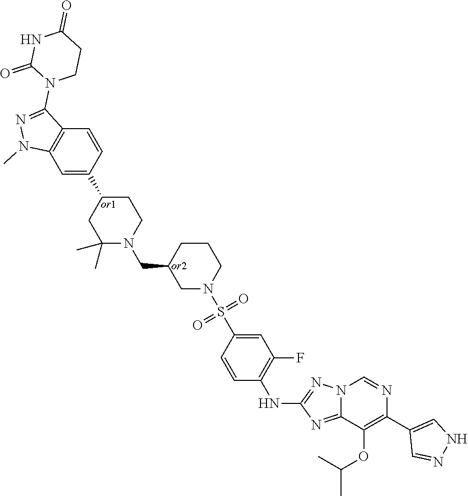 [SMILES: CC(C)Oc1c(-c2cn[nH]c2)ncn2nc(Nc3ccc(S(=O)(=O)N4CCC[C@H](CN5CC[C@@H](c6ccc7c(N8CCC(=O)NC8=O)nn(C)c7c6)CC5(C)C)C4)cc3F)nc12] |
| 103 | 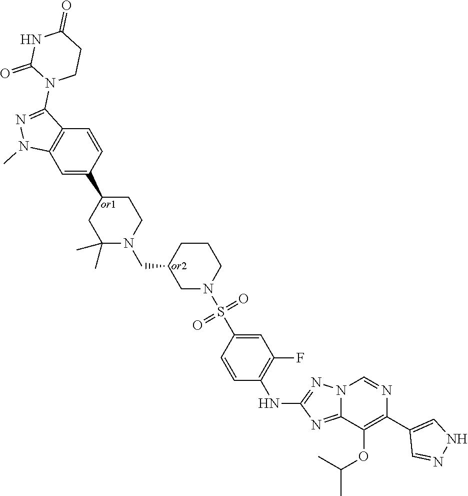 [SMILES: CC(C)Oc1c(-c2cn[nH]c2)ncn2nc(Nc3ccc(S(=O)(=O)N4CCC[C@@H](CN5CC[C@H](c6ccc7c(N8CCC(=O)NC8=O)nn(C)c7c6)CC5(C)C)C4)cc3F)nc12] |
| 104 | 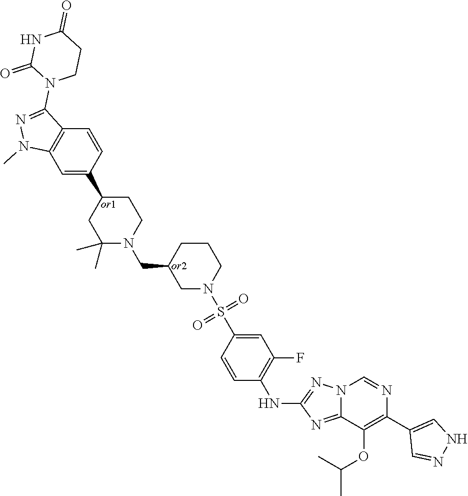 [SMILES: CC(C)Oc1c(-c2cn[nH]c2)ncn2nc(Nc3ccc(S(=O)(=O)N4CCC[C@H](CN5CC[C@H](c6ccc7c(N8CCC(=O)NC8=O)nn(C)c7c6)CC5(C)C)C4)cc3F)nc12] |
| 105 | 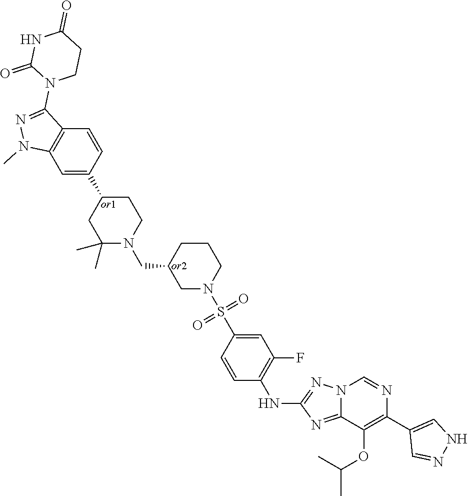 [SMILES: CC(C)Oc1c(-c2cn[nH]c2)ncn2nc(Nc3ccc(S(=O)(=O)N4CCC[C@@H](CN5CC[C@@H](c6ccc7c(N8CCC(=O)NC8=O)nn(C)c7c6)CC5(C)C)C4)cc3F)nc12] |
| 106 | 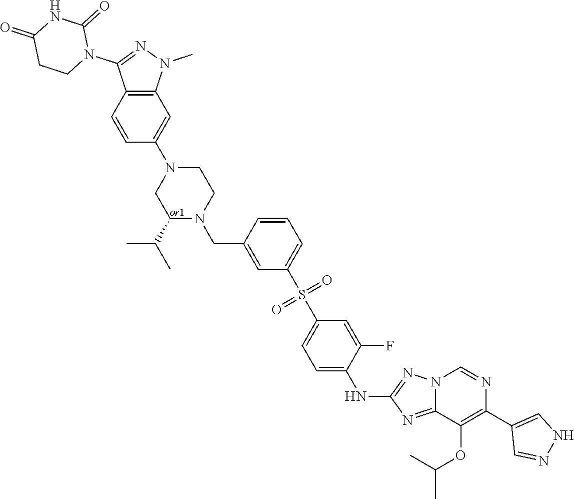 [SMILES: CC(C)Oc1c(-c2cn[nH]c2)ncn2nc(Nc3ccc(S(=O)(=O)c4cccc(CN5CCN(c6ccc7c(N8CCC(=O)NC8=O)nn(C)c7c6)C[C@H]5C(C)C)c4)cc3F)nc12] |
| 107 | 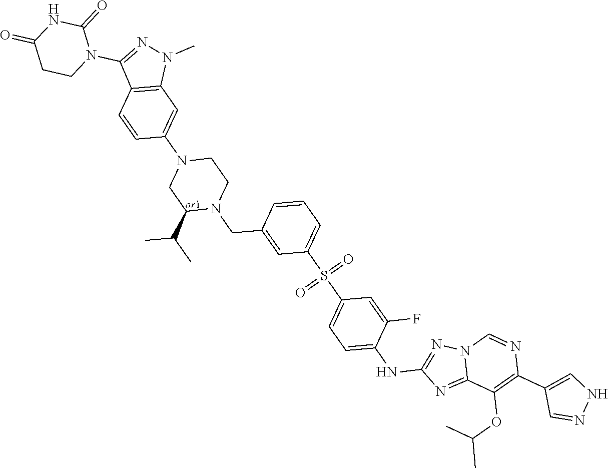 [SMILES: CC(C)Oc1c(-c2cn[nH]c2)ncn2nc(Nc3ccc(S(=O)(=O)c4cccc(CN5CCN(c6ccc7c(N8CCC(=O)NC8=O)nn(C)c7c6)C[C@@H]5C(C)C)c4)cc3F)nc12] |
| 108 | 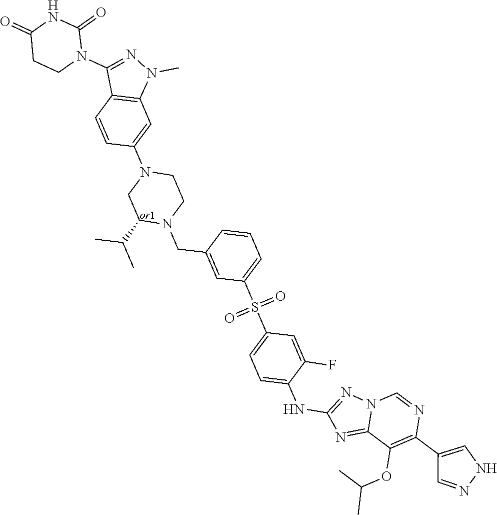 [SMILES: CC(C)Oc1c(-c2cn[nH]c2)ncn2nc(Nc3ccc(S(=O)(=O)c4cccc(CN5CCN(c6ccc7c(N8CCC(=O)NC8=O)nn(C)c7c6)C[C@H]5C(C)C)c4)cc3F)nc12] |
| 109 | 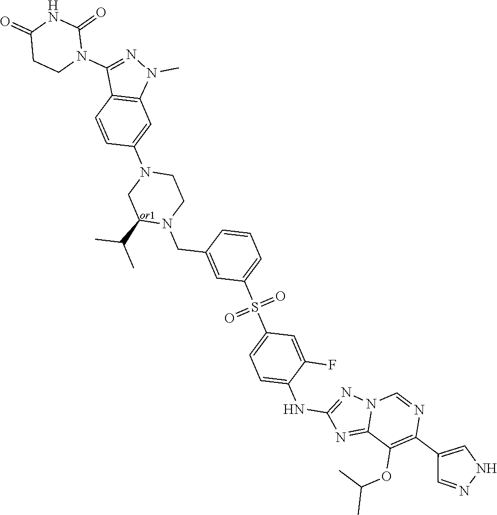 [SMILES: CC(C)Oc1c(-c2cn[nH]c2)ncn2nc(Nc3ccc(S(=O)(=O)c4cccc(CN5CCN(c6ccc7c(N8CCC(=O)NC8=O)nn(C)c7c6)C[C@@H]5C(C)C)c4)cc3F)nc12] |

Structure, physicochemical data for compounds 1 to 109

| Cpd | Observed | |
|---|---|---|
| No. | [M + H]⁺ | ¹H NMR Data |
| 1 | 822.2 | ¹H NMR (400 MHz, dimethyl sulfoxide-d₆) δ |
| | | 13.11 (s, 1H), 10.54 (s, 1H), 8.96 (s, 1H), 8.28 (s, |
| | | 1H), 8.11 (s, 1H), 7.83 − 7.61 (m, 4H), 7.52 (d, J = |
| | | 8.5 Hz, 1H), 7.42 (s, 1H), 7.03 (dd, J = 20.9, 7.8 |
| | | Hz, 2H), 5.46 (p, J = 6.3 Hz, 1H), 3.91 (d, J = |
| | | 15.3 Hz, 5H), 3.65 (s, 2H), 3.56 − 3.44 (m, 4H), |
| | | 2.95 (d, J = 10.7 Hz, 2H), 2.74 (t, J = 6.6 Hz, 2H), |
| | | 2.63 (d, J = 10.3 Hz, 3H), 2.14 (s, 2H), 1.99 (d, J = |
| | | 12.1 Hz, 2H), 1.79 (d, J = 8.3 Hz, 3H), 1.62 (d, |
| | | J = 10.9 Hz, 2H), 1.25 (d. J = 6.2 Hz, 6H). |
| 2 | 835.3 | ¹H NMR (400 MHz, dimethyl sulfoxide-d₆) δ |
| | | 8.97 (s, 1H), 8.19 (s, 2H), 7.69 − 7.59 (m, 4H), |
| | | 7.47 (d, J = 8.3 Hz, 1H), 6.56 − 6.39 (m, 2H), |
| | | 5.44 (p, J = 6.1 Hz, 1H), 4.96 (dd, J = 13.3, 5.1 |
| | | Hz, 1H), 4.27 (d, J = 17.1 Hz, 1H), 4.14 (d, J = |
| | | 17.1 Hz, 1H), 3.96 (s, 4H), 3.67 (s, 2H), 3.49 (d, |
| | | J = 11.2 Hz, 3H), 3.36 (s, 4H), 2.84 (ddd, J = 17.8, |
| | | 13.4, 5.4 Hz, 1H), 2.58 (d, J = 26.2 Hz, 3H), 2.40 |
| | | 2.23 (m, 1H), 1.97 (t, J = 15.6 Hz, 3H), 1.58 (q, |
| | | J = 10.2, 9.7 Hz, 2H), 1.25 (d, J = 6.1 Hz, 6H). |
| 3 | 841.4 | ¹H NMR (400 MHz, dimethyl sulfoxide-d₆) δ |
| | | 13.11 (s, 1H), 10.97 (s, 1H), 9.03 (s, 1H), 8.30 (s, |
| | | 1H), 8.13 (s, 1H), 7.80 − 7.58 (m, 4H), 7.39 (d, J = |
| | | 11.6 Hz, 1H), 7.21 (d, J = 7.5 Hz, 1H), 7.08 (d, |
| | | J = 7.1 Hz, 1H), 5.60 − 5.39 (m, 1H), 5.06 (dd, J = |
| | | 13.3, 5.1 Hz, 1H), 4.44 − 4.13 (m, 2H), 3.77 − |
| | | 3.62 (m, 2H), 3.52 − 3.44 (m, 3H), 3.13 (s, 4H), |
| | | 2.89 (ddd, J = 18.1, 13.5, 5.4 Hz, 1H), 2.75 − 2.54 |
| | | (m, 7H), 2.35 (m, 1H), 2.09 − 1.89 (m, 3H), 1.59 |
| | | (q, J = 10.8, 10.1 Hz, 2H), 1.27 (d, J = 6.1 Hz, |
| | | 6H). |
| 4 | 794.4 | ¹H NMR (400 MHz, dimethyl sulfoxide-d₆) δ |
| | | 8.90 (s, 1H), 8.20 (s, 2H), 7.73 − 7.56 (m, 5H), |
| | | 7.52 (s, 1H), 7.44 (d, J = 7.9 Hz, 1H), 5.47 − 5.29 |
| | | (m, 1H), 5.00 (dd, J = 13.3, 5.1 Hz, 1H), 4.40 (d, |
| | | J = 17.5 Hz, 1H), 4.28 (d, J = 17.5 Hz, 1H), 3.81 − |
| | | 3.70 (m, 3H), 3.67 (t, J = 7.5 Hz, 2H), 3.55 − |
| | | 3.34 (m, 3H), 3.19 (t, J = 6.9 Hz, 2H), 2.93 − 2.74 |
| | | (m, 1H), 2.69 − 2.55 (m, 3H), 2.44 − 2.27 (m, |
| | | 1H), 2.10 − 1.88 (m, 3H), 1.61 − 1.53 (m, 2H), |
| | | 1.21 (d, J = 6.1 Hz, 6H). |
| 5 | 836.3 | ¹H NMR (400 MHz, dimethyl sulfoxide-d₆) δ |
| | | 13.11 (s, 1H), 10.54 (s, 1H), 8.96 (s, 1H), 8.33 − |
| | | 8.04 (m, 2H), 7.81 − 7.60 (m, 5H), 7.55 − 7.38 (m, |
| | | 2H), 7.13 − 6.91 (m, 2H), 5.53-5.41 (m, 1H), 3.96 |
| | | 3.83 (m, 5H), 3.7 − 3.56 (m, 3H), 3.09-2.92 (m, |
| | | 6H), 2.77 − 2.57 (m, 3H), 2.24 − 2.07 (m, 3H), |
| | | 1.97 − 1.66 (m, 6H), 1.29 − 1.16 (m, 6H), 0.91 (d, |
| | | J = 6.8 Hz, 3H). |
| 6 | 844.3 | ¹H NMR (400 MHz, dimethyl sulfoxide-d₆) δ |
| | | 13.05 (s, 1H), 11.02 (s, 1H), 9.04 (s, 1H), 8.17 (d, |
| | | J = 61.4 Hz, 2H), 7.05 (d, J = 7.6 Hz, 1H), 6.97 − |
| | | 6.85 (m, 3H), 5.51 − 5.42 (m, 1H), 5.38 − 5.22 (m, |
| | | 1H), 3.51 (s, 6H), 2.91 (t, J = 11.8 Hz, 5H), 2.84 − |
| | | 2.48 (m, 5H), 2.13 (d, J = 7.1 Hz, 2H), 1.95 (t, J = |
| | | 14.0 Hz, 6H), 1.71 (s, 7H), 1.48 (d, J = 11.1 Hz, |
| | | 2H), 1.24 (d, J = 6.0 Hz, 6H), 1.05 (d, J = 12.3 |
| | | Hz, 2H |
| 7 | 832.3 | ¹H NMR (400 MHz, dimethyl sulfoxide-d₆) δ |
| | | 13.13 (s, 1H), 11.10 (s, 1H), 9.09 (s, 1H), 8.40- |
| | | 8.09 (m, 2H), 7.21 (d, J = 8.0 Hz, 1H), 7.06-6.91 |
| | | (m, 3H), 5.62 − 5.33 (m, 2H), 3.90 − 3.79 (m, |
| | | 1H), 3.58 (s, 3H), 3.30 − 2.76 (m, 11H), 2.74 − |
| | | 2.56 (m, 3H), 2.38-2.31 (m, 2H), 2.24-2.14 (m, |
| | | 1H), 2.11 − 1.93 (m, 3H), 1.88 − 1.65 (m, 7H), |
| | | 1.34 − 1.19 (m, 8H), 0.93 (d, J = 6.9 Hz, 3H). |
| 8 | 833.2 | ¹H NMR (400 MHz, dimethyl sulfoxide-d₆) δ |
| | | 13.19 (s, 1H), 10.55 (s, 1H), 10.24 (s, 1H), 9.29 |
| | | (d, J = 2.6 Hz, 1H), 8.53 (t, J = 8.4 Hz, 1H), 8.44 |
| | | 8.10 (m, 2H), 7.95 (d, J = 2.2 Hz, 1H), 7.90 − |
| | | 7.82 (m, 3H), 7.68 − 7.40 (m, 4H), 7.04 (d, J = 8.4 |
| | | Hz, 1H), 5.68-5.51 (m, 1H), 3.96 (s, 3H), 3.90 (t, |
| | | J = 6.8 Hz, 2H), 3.62 (s, 2H), 2.91 (d, J = 10.8 |
| | | Hz, 2H), 2.74 (t, J = 6.8 Hz, 2H), 2.69 − 2.60 (m, |
| | | 1 H), 2.15 − 2.03 (m, 2H), 1.85 − 1.72 (m, 4H), |
| | | 1.34 (d, J = 6.2 Hz, 6H). |
| 9 | 917.2 | ¹H NMR (400 MHz, dimethyl sulfoxide-d₆) δ |
| | | 13.20 (s, 1H), 10.95 (s, 1H), 10.21 (s, 1H), 9.34 |
| | | (s, 1H), 8.50 (t, J = 8.4 Hz, 1H), 8.38 (s, 1H), 8.20 |
| | | (s, 1H), 7.89 − 7.82 (m, 2H), 7.50 (d, J = 8.4 Hz, |
| | | 1H), 7.46 − 7.38 (m, 2H), 7.31 (d, J = 7.8 Hz, |
| | | 1H), 7.24 − 7.18 (m, 1H), 7.04 (d, J = 8.8 Hz, |
| | | 2H), 5.68 − 5.59 (m, 1H), 5.10 − 4.97 (m, 1H), |
| | | 4.43 − 4.14 (m, 2H), 3.88 (d, J = 12.4 Hz, 2H), |
| | | 3.23 (s, 4H), 2.95 − 2.79 (m, 3H), 2.71 − 2.57 (m, |
| | | 3H), 2.47 − 2.33 (m, 2H), 2.21 (d, J = 6.6 Hz, |
| | | 2H), 2.02 − 1.92 (m, 1H), 1.81 (d, J = 11.8 Hz, |
| | | 3H), 1.36 (d, J = 6.1 Hz, 7H), 1.27 − 1.08 (m, |
| | | 2H). |
| 10 | 846.1 | ¹H NMR (400 MHz, dimethyl sulfoxide-d₆) δ |
| | | 13.18 (s, 1H), 10.93 (s, 1H), 10.22 (s, 1H), 9.32 |
| | | (s, 1H), 8.52 (t, J = 8.3 Hz, 1H), 8.28 (d, J = 68.0 |
| | | Hz, 2H), 7.85 (dt, J = 8.4, 2.8 Hz, 4H), 7.69 − |
| | | 7.52 (m, 2H), 7.46 (d, J = 8.2 Hz, 1H), 6.55 − |
| | | 6.37 (m, 2H), 5.61 (hept, J = 6.2 Hz, 1H), 5.02 |
| | | (dd, J = 13.3, 5.1 Hz, 1H), 4.33 − 4.09 (m, 2H), |
| | | 3.98 (s, 4H), 3.64 (s, 2H), 3.33 (s, 4H), 2.89 (ddd, |
| | | J = 18.1, 13.7, 5.4 Hz, 1H), 2.62 − 2.53 (m, 1H), |
| | | 2.34 (tt, J = 13.3, 6.6 Hz, 1H), 2.01 − 1.89 (m, |
| | | 1H), 1.35 (d, J = 6.1 Hz, 6H). |
| 11 | 852.2 | ¹H NMR (400 MHz, dimethyl sulfoxide-d₆) δ |
| | | 13.18 (s, 1H), 10.97 (s, 1H), 10.22 (d, J = 2.0 Hz, |
| | | 1H), 9.31 (s, 1H), 8.52 (t, J = 8.5 Hz, 1H), 8.37 (s, |
| | | 1H), 8.20 (s, 1H), 7.97 − 7.81 (m, 4H), 7.75 − |
| | | 7.48 (m, 2H), 7.40 (d, J = 11.5 Hz, 1H), 7.22 (d, |
| | | J = 7.5 Hz, 1H), 5.62 (hept, J = 6.2 Hz, 1H), 5.06 |
| | | (dd, J = 13.3, 5.1 Hz, 1H), 4.44 − 4.13 (m, 2H), |
| | | 3.65 (s, 2H), 3.13 (d, J = 5.4 Hz, 4H), 2.90 (ddd, |
| | | J = 17.7, 13.4, 5.4 Hz, 1H), 2.55 (d, J = 6.2 Hz, |
| | | 5H), 2.36 (qd, J = 13.1, 4.5 Hz, 1H), 2.07 − 1.89 |
| | | (m, 1H), 1.35 (d, J = 6.1 Hz, 6H). |
| 12 | 805.2 | ¹H NMR (400 MHz, dimethyl sulfoxide-d₆) δ |
| | | 13.19 (s, 1H), 10.99 (s, 1H), 10.24 (s, 1H), 9.32 |
| | | (s, 1H), 8.52 (t, J = 8.5 Hz, 1H), 8.37 (s, 1H), 8.20 |
| | | (s, 1H), 8.00 − 7.78 (m, 4H), 7.73 − 7.54 (m, 4H), |
| | | 7.53 − 7.44 (m, 1H), 5.62 (p, J = 6.1 Hz, 1H), |
| | | 5.10 (dd, J = 13.3, 5.1 Hz, 1H), 4.43 (d, J = 17.3 |
| | | Hz, 1H), 4.31 (d, J = 17.3 Hz, 1H), 3.88 − 3.63 |
| | | (m, 5H), 3.25 − 3.12 (m, 2H), 2.91 (ddd, J = 17.9, |
| | | 13.5, 5.4 Hz, 1H), 2.69 − 2.56 (m, 1H), 2.39 (qd, |
| | | J = 13.2, 4.5 Hz, 1H), 2.06 − 1.86 (m, 1H), 1.35 (d, |
| | | J = 6.1 Hz, 6H). |
| 13 | 848.3 | ¹H NMR (400 MHz, dimethyl sulfoxide-d₆) δ |
| | | 13.19 (s, 1H), 11.09 (s, 1H), 10.24 (s, 1H), 9.31 |
| | | (s, 1H), 8.53 (t, J = 8.5 Hz, 1H), 8.37 (s, 1H), 8.19 |
| | | (s, 1H), 8.01 − 7.79 (m, 4H), 7.62 (dt, J = 15.2, |
| | | 7.6 Hz, 2H), 7.11 − 6.84 (m, 3H), 5.61 (hept, J = |
| | | 6.1 Hz, 1H), 5.35 (dd, J = 12.6, 5.4 Hz, 1H), 3.59 |
| | | (d, J = 30.0 Hz, 5H), 3.22 (d, J = 5.9 Hz, 1H), |
| | | 2.87 (t, J = 15.9 Hz, 3H), 2.77 − 2.55 (m, 2H), |
| | | 2.14 (q, J = 8.9, 7.8 Hz, 2H), 2.02 − 1.92 (m, 1H), |
| | | 1.78 (d, J = 7.7 Hz, 4H), 1.34 (d, J = 6.1 Hz, 6H). |
| 14 | 855.4 | ¹H NMR (400 MHz, dimethyl sulfoxide-d₆) δ |
| | | 13.20 (s, 1H), 11.09 (s, 1H), 10.43 − 9.98 (m, 1H), |
| | | 9.35 (s, 1H), 8.55 (t, J = 8.5 Hz, 1H), 8.30 (s, 2H), |
| | | 7.68 − 7.50 (m, 2H), 7.09 − 6.85 (m, 3H), 5.73 − |
| | | 5.57 (m, J = 6.2, 5.7 Hz, 1H), 5.36 (dd, J = 12.6, |
| | | 5.4 Hz, 1H), 3.74 − 3.60 (m, 2H), 3.55 (s, 3H), |
| | | 3.25 − 3.08 (m, 1H), 2.88 (t, J = 11.1 Hz, 3H), |
| | | 2.78 − 2.54 (m, 2H), 2.37 − 2.19 (m, 2H), 2.12 (d, |
| | | J = 7.0 Hz, 2H), 1.98 (dt, J = 11.2, 6.3 Hz, 3H), |
| | | 1.88 − 1.61 (m, 6H), 1.59 − 1.46 (m, 1H), 1.38 (d, |
| | | J = 6.1 Hz, 6H), 1.21 − 1.03 (m, 2H), 0.90 − 0.76 |
| | | (m, 1H). |
| 15 | 814.4 | ¹H NMR (400 MHz, dimethyl sulfoxide-d₆) δ |
| | | 13.15 (s, 1H), 10.52 (s, 1H), 10.14 (s, 1H), 9.33 |
| | | (s, 1H), 8.53 (t, J = 8.5 Hz, 1H), 8.29 (s, 2H), 7.64 |
| | | (ddd, J = 10.7, 7.9, 2.1 Hz, 2H), 7.53 (d, J = 8.4 |
| | | Hz, 1H), 7.42 (s, 1H), 7.02 (d, J = 8.5 Hz, 1H), |
| | | 5.64 (p, J = 6.2 Hz, 1H), 3.95 (s, 3H), 3.90 (t, J = |
| | | 6.7 Hz, 2H), 3.08 − 2.90 (m, 4H), 2.75 (d, J = 6.6 |
| | | Hz, 1H), 2.73 (s, 3H), 2.62 (s, 1H), 2.33 (s, 2H), |
| | | 2.00 (t, J = 10.9 Hz, 2H), 1.77 (d, J = 9.3 Hz, 3H), |
| | | 1.73 − 1.61 (m, 2H), 1.37 (d, J = 6.2 Hz, 6H), |
| | | 1.23 (s, 2H). |
| 16 | 829.3 | ¹H NMR (400 MHz, dimethyl sulfoxide-d₆) δ |
| | | 13.20 (s, 1H), 11.09 (s, 1H), 10.18 (s, 1H), 9.34 |
| | | (s, 1H), 8.54 (t, J = 8.5 Hz, 1H), 8.29 (d, J = 69.7 |
| | | Hz, 2H), 7.75 − 7.54 (m, 2H), 7.09 (d, J = 1.6 Hz, |
| | | 1H), 6.99 (d, J = 8.2 Hz, 1H), 6.90 (dd, J = 8.2, |
| | | 1.6 Hz, 1H), 5.65 (hept, J = 6.2 Hz, 1H), 5.33 (dd, |
| | | J = 12.8, 5.4 Hz, 1H), 3.32 (s, 3H), 3.08 − 2.84 |
| | | (m, 5H), 2.76 − 2.53 (m, 5H), 2.31 (t, J = 7.1 Hz, |
| | | 2H), 1.96 (tt, J = 11.5, 4.3 Hz, 3H), 1.81 − 1.60 |
| | | (m, 6H), 1.37 (d, J = 6.1 Hz, 6H). |
| 17 | 830.2 | ¹H NMR (300 MHz, dimethyl sulfoxide-d₆) δ |
| | | 13.19 (s, 1H), 11.08 (s, 1H), 10.17 (s, 1H), 9.34 |
| | | (s, 1H), 8.54 (t, J = 8.5 Hz, 1H), 8.29 (d, J = 45.9 |
| | | Hz, 2H), 7.65 (ddd, J = 10.9, 5.9, 2.2 Hz, 2H), |
| | | 7.02 − 6.79 (m, 3H), 5.65 (p, J = 6.2 Hz, 1H), |
| | | 5.34 (dd, J = 12.6, 5.4 Hz, 1H), 3.61 (s, 3H), 3.03 |
| | | (t, J = 7.1 Hz, 2H), 2.98 − 2.78 (m, 6H), 2.72 (s, |
| | | 4H), 2.70 − 2.56 (m, 3H), 2.36 (t, J = 7.0 Hz, 2H), |
| | | 2.31 − 2.09 (m, 1H), 2.05 − 1.91 (m, 1H), 1.67 (t, |
| | | J = 7.2 Hz, 2H), 1.37 (d, J = 6.2 Hz, 6H). |
| 18 | 829.4 | ¹H NMR (400 MHz, dimethyl sulfoxide-d₆) δ |
| | | 13.19 (s, 1H), 11.08 (s, 1H), 10.16 (s, 1H), 9.34 |
| | | (s, 1H), 8.53 (s, 1H), 8.30 (d, J = 58.2 Hz, 2H), |
| | | 7.65 (s, 2H), 6.98 (s, 3H), 5.73 − 5.54 (m, 1H), |
| | | 5.36 (s, 1H), 3.57 (s, 2H), 3.24 − 3.13 (m, 2H), |
| | | 3.08 − 2.83 (m, 5H), 2.72 (s, 4H), 2.33 (s, 3H), |
| | | 2.16 − 1.90 (m, 3H), 1.71 (d, J = 38.8 Hz, 6H), |
| | | 1.37 (s, 5H). |
| 19 | 818.3 | ¹H NMR (400 MHz, dimethyl sulfoxide-d₆) δ |
| | | 13.11 (s, 1H), 11.08 (s, 1H), 9.10 (s, 1H), 8.38- |
| | | 8.04 (m, 2H), 7.27 − 6.86 (m, 4H), 5.64 − 5.24 (m, |
| | | 2H), 3.71-3.47 (m, 6H), 3.26-3.17(m, 3H), 3.05- |
| | | 2.97 (m, 9H), 2.39-2.30 (m, 2H), 2.12-1.94 (m, |
| | | 6H), 1.86-1.68 (m, 6H), 1.62-1.49 (m, 2H), 1.31 |
| | | (d, J = 4.0 Hz, 6H). |
| 20 | 810.3 | ¹H NMR (400 MHz, dimethyl sulfoxide-d₆) δ |
| | | 13.12 (s, 1H), 10.99 (s, 1H), 9.06 (s, 1H), 8.30 |
| | | (s, 1H), 8.14 (s, 1H), 7.67 (d, J = 7.8 Hz, 1H), |
| | | 7.61 − 7.50 (m, 2H), 7.44 (d, J = 7.9 Hz, 1H), |
| | | 7.33 (d, J = 7.7 Hz, 1H), 7.21 (dd, J = 8.2, 2.5 |
| | | Hz, 1H), 7.09 (d, J = 7.5 Hz, 2H), 5.62 − 5.30 |
| | | (m, 1H), 5.11 (dd, J = 13.3, 5.1 Hz, 1H), 4.97 |
| | | (p, J = 5.6 Hz, 1H), 4.44 (d, J = 17.2 Hz, 1H), |
| | | 4.31 (d, J = 17.3 Hz, 1H), 3.89 − 3.68 (m, 4H), |
| | | 3.67 − 3.42 (m, 3H), 3.16 (dd, J = 7.9, 5.5 Hz, |
| | | 2H), 3.02 − 2.81 (m, 1H), 2.70 − 2.55 (m, 3H), |
| | | 2.46 − 2.27 (m, 1H), 2.12 − 1.91 (m, 3H), 1.59 |
| | | (q, J = 9.2, 7.8 Hz, 2H), 1.28 (d, J = 6.1 Hz, 6H). |
| 21 | 875.3 | ¹H NMR (400 MHz, dimethyl sulfoxide-d₆) δ |
| | | 13.18 (s, 1H), 10.94 (s, 1H), 10.18 (s, 1H), 9.32 |
| | | (s, 1H), 8.50 (t, J = 8.3 Hz, 1H), 8.28 (d, J = |
| | | 65.4 Hz, 2H), 7.87 − 7.75 (m, 2H), 7.52 (d, J = |
| | | 8.6 Hz, 1H), 7.38 (t, J = 7.9 Hz, 1H), 7.20 (d, J = |
| | | 7.7 Hz, 1H), 7.12 − 7.03 (m, 2H), 6.93 (s, |
| | | 1H), 6.68 (dd, J = 8.1, 2.3 Hz, 1H), 5.63 (p, J = |
| | | 6.1 Hz, 1H), 5.04 (dd, J = 13.3, 5.1 Hz, 1H), |
| | | 4.33 (d, J = 16.9 Hz, 1H), 4.20 (d, J = 16.9 Hz, |
| | | 1H), 4.02 (t, J = 7.3 Hz, 2H), 3.75 (dd, J = 8.0, |
| | | 5.1 Hz, 2H), 3.40 − 3.32 (m, 1H), 3.34 − 3.33 |
| | | (m, 6H), 2.90 (ddd, J = 18.0, 13.3, 5.4 Hz, 1H), |
| | | 2.69 − 2.55 (m, 1H), 2.55 − 2.53 (m, 2H), 2.44 − |
| | | 2.26 (m, 1H), 2.06 − 1.87 (m, 1H), 1.36 (d, J = |
| | | 6.1 Hz, 6H). |
| 22 | 917.2 | ¹H NMR (400 MHz, dimethyl sulfoxide-d₆) δ |
| | | 13.18 (s, 1H), 10.94 (s, 1H), 10.18 (s, 1H), 9.33 |
| | | (s, 1H), 8.50 (t, J = 8.5 Hz, 1H), 8.41 − 8.16 (m, |
| | | 2H), 7.85 (dt, J = 10.4, 2.6 Hz, 2H), 7.52 (d, J = |
| | | 8.4 Hz, 1H), 7.44 − 7.34 (m, 2H), 7.26 (d, J = |
| | | 7.7 Hz, 1H), 7.20 (dd, J = 8.4, 2.4 Hz, 1H), 7.05 |
| | | (d, J = 8.6 Hz, 2H), 5.74 − 5.51 (m, 1H), 5.05 |
| | | (dd, J = 13.3, 5.1 Hz, 1H), 4.39 − 4.10 (m, 2H), |
| | | 3.79 (d, J = 12.2 Hz, 2H), 3.32 − 3.20 (m, 5H), |
| | | 2.97 − 2.83 (m, 1H), 2.76 (t, J = 11.9 Hz, 2H), |
| | | 2.64 − 2.55 (m, 1H), 2.37 (qd, J = 13.2, 4.8 Hz, |
| | | 1H), 2.21 (d, J = 6.9 Hz, 2H), 1.97 (dd, J = 11.7, |
| | | 5.7 Hz, 1H), 1.89 − 1.63 (m, 3H), 1.36 (d, J = |
| | | 6.1 Hz, 6H), 1.27 − 1.05 (m, 4H), 0.92 − 0.71 |
| | | (m, 1H). |
| 23 | 890.5 | ¹H NMR (400 MHz, dimethyl sulfoxide-d₆) δ |
| | | 13.20 (s, 1H), 10.95 (s, 1H), 10.25 (s, 1H), 9.34 |
| | | (s, 1H), 8.52 (t, J = 8.4 Hz, 1H), 8.38 (s, 1H), |
| | | 8.20 (s, 1H), 7.89 (ddd, J = 13.1, 9.5, 2.2 Hz, |
| | | 2H), 7.60 − 7.44 (m, 3H), 7.35 (s, 1H), 7.16 (d, |
| | | J = 7.2 Hz, 1H), 7.09 − 6.94 (m, 2H), 5.70 − |
| | | 5.56 (m, 1H), 5.04 (dd, J = 13.2, 5.1 Hz, 1H), |
| | | 4.97 − 4.85 (m, 1H), 4.30 (d, J = 16.8 Hz, 1H), |
| | | 4.18 (d, J = 16.9 Hz, 1H), 3.81 − 3.63 (m, 4H), |
| | | 3.06 − 2.82 (m, 5H), 2.69 − 2.56 (m, 1H), 2.43 − |
| | | 2.22 (m, 2H), 2.00 − 1.84 (m, 1H), 1.78 − 1.59 |
| | | (m, 2H), 1.36 (d, J = 6.1 Hz, 6H), 1.32 − 1.17 |
| | | (m, 2H). |
| 24 | 814.2 | ¹H NMR (300 MHz, dimethyl sulfoxide-d₆) δ |
| | | 13.20 (s, 1H), 10.55 (s, 1H), 10.18 (s, 1H), 9.35 |
| | | (s, 1H), 8.55 (t, J = 8.5 Hz, 1H), 8.38 (s, 1H), |
| | | 8.21 (s, 1H), 7.71 − 7.57 (m, 2H), 7.45 (dd, J = |
| | | 8.1, 1.0 Hz, 1H), 7.27 (d, J = 7.1 Hz, 1H), 7.06 |
| | | (t, J = 7.6 Hz, 1H), 5.65 (p, J = 6.1 Hz, 1H), |
| | | 4.19 (s, 3H), 3.87 (t, J = 6.7 Hz, 2H), 3.29 (m, 1 |
| | | H), 3.02 (q, J = 11.1, 9.1 Hz, 4H), 2.81 − 2.70 |
| | | (m, 5H), 2.36 (t, J = 7.0 Hz, 2H), 2.18 − 2.04 |
| | | (m, 2H), 1.88 (d, J = 12.7 Hz, 2H), 1.83 − 1.59 |
| | | (m, 4H), 1.37 (d, J = 6.2 Hz, 6H). |
| 25 | 848.3 | ¹H NMR (400 MHz, dimethyl sulfoxide-d₆) δ |
| | | 13.19 (s, 1H), 11.09 (s, 1H), 10.24 (d, J = 1.9 |
| | | Hz, 1H), 9.29 (s, 1H), 8.52 (t, J = 8.5 Hz, 1H), |
| | | 8.28 (d, J = 70.8 Hz, 2H), 7.97 − 7.81 (m, 4H), |
| | | 7.67 − 7.53 (m, 2H), 7.12 (d, J = 1.5 Hz, 1H), |
| | | 7.05 − 6.89 (m, 2H), 5.61 (m, 1H), 5.33 (dd, J = |
| | | 12.7, 5.4 Hz, 1H), 3.60 (s, 2H), 3.33 (s, 3H), |
| | | 3.27 (d, J = 13.2 Hz, 1H), 2.95 − 2.81 (m, 3H), |
| | | 2.76 − 2.54 (m, 2H), 2.14 − 1.93 (m, 3H), 1.72 |
| | | (d, J = 9.5 Hz, 4H), 1.35 (dd, J = 6.2, 1.8 Hz, |
| | | 6H). |
| 26 | 833.4 | ¹H NMR (400 MHz, dimethyl sulfoxide-d₆) δ |
| | | 13.39 − 12.73 (m, 1H), 10.54 (s, 1H), 10.25 (s, |
| | | 1H), 9.32 (s, 1H), 8.53 (t, J = 8.5 Hz, 1H), 8.37 |
| | | (s, 1H), 8.19 (s, 1H), 7.91 − 7.81 (m, 3H), 7.90 |
| | | (s, 1H), 7.71 − 7.56 (m, 2H), 7.44 (d, J = 8.1 |
| | | Hz, 1H), 7.29 (d, J = 7.1 Hz, 1H), 7.05 (t, J = |
| | | 7.6 Hz, 1H), 5.61 (hept, J = 6.2 Hz, 1H), 4.17 |
| | | (s, 3H), 3.85 (t, J = 6.6 Hz, 2H), 3.65 (s, 2H), |
| | | 3.30 (d, J = 13.0 Hz, 1H), 2.93 (d, J = 10.8 Hz, |
| | | 2H), 2.74 (t, J = 6.6 Hz, 2H), 2.20 (t, J = 11.1 |
| | | Hz, 2H), 1.82 (dt, J = 35.7, 12.1 Hz, 4H), 1.34 |
| | | (d, J = 6.1 Hz, 6H). |
| 27 | 843.4 | ¹H NMR (300 MHz, dimethyl sulfoxide-d₆) δ |
| | | 13.19 (s, 1H), 11.09 (s, 1H), 10.24 (s, 1H), 9.33 |
| | | (s, 1H), 8.61 (t, J = 8.3 Hz, 1H), 8.29 (s, 2H), |
| | | 7.71 (d, J = 9.2 Hz, 2H), 6.89 (t, J = 8.2 Hz, |
| | | 1H), 6.70 (dd, J = 8.1, 4.9 Hz, 2H), 5.64 (p, J = |
| | | 6.2 Hz, 1H), 5.31 (dd, J = 12.5, 5.3 Hz, 1H), |
| | | 4.06 (t, J = 4.9 Hz, 2H), 3.49 (s, 3H), 3.49 − |
| | | 3.40 (m, 5H), 2.93 − 2.54 (m, 5H), 2.32 − 2.15 |
| | | (m, 4H), 2.05 − 1.79 (m, 1H), 1.36 (d, J = 6.1 |
| | | Hz, 6H), 1.35 − 1.27 (m, 1H), 1.28 − 1.12 (m, |
| | | 2H). |
| 28 | 843.3 | ¹H NMR (400 MHz, dimethyl sulfoxide-d₆) δ |
| | | 13.19 (s, 1H), 10.16 (s, 1H), 9.34 (s, 1H), 8.60 − |
| | | 8.17 (m, 3H), 7.77 − 7.50 (m, 2H), 6.98 (td, J = |
| | | 6.9, 3.3 Hz, 3H), 5.75 − 5.36 (m, 2H), 3.57 (s, |
| | | 3H), 3.27 − 3.15 (m, 1H), 3.03 (s, 5H), 3.00 − |
| | | 2.91 (m, 3H), 2.82 − 2.74 (m, 1H), 2.72 (s, 3H), |
| | | 2.71 − 2.63 (m, 1H), 2.41 − 2.28 (m, 2H), 2.14 − |
| | | 1.92 (m, 3H), 1.84 − 1.69 (m, 4H), 1.69 − 1.60 |
| | | (m, 2H), 1.37 (d, J = 6.1 Hz, 6H). |
| 29 | 804.3 | N/A |
| 30 | 804.3 | N/A |
| 31 | 819.3 | ¹H NMR (400 MHz, dimethyl sulfoxide-d₆) δ |
| | | 13.12 (s, 1H), 11.01 (s, 1H), 10.11 (s, 1H), 9.24 |
| | | (s, 1H), 8.44 (t, J = 8.5 Hz, 1H), 8.21 (s, 2H), 7.78 |
| | | (d, J = 16.7 Hz, 4H), 7.55 − 7.39 (m, 2H), 7.09 − |
| | | 6.71 (m, 3H), 5.55 (dt, J = 12.7, 6.5 Hz, 1H), 5.34 − |
| | | 5.15 (m, 1H), 3.62 (s, 3H), 2.92 (d, J = 7.8 Hz, |
| | | 1H), 2.79 (t, J = 13.4 Hz, 2H), 2.58 (q, J = 18.7, |
| | | 15.6 Hz, 4H), 2.30 (d, J = 8.1 Hz, 1H), 2.20 − |
| | | 1.99 (m, 2H), 1.92 (s, 1H), 1.76 (d, J = 10.3 Hz, |
| | | 1H), 1.28 (d, J = 6.1 Hz, 6H). |
| 32 | 792.2 | ¹H NMR (400 MHz, dimethyl sulfoxide-d₆) δ |
| | | 13.19 (s, 1H), 10.86 (d, J = 3.5 Hz, 1H), 10.22 |
| | | (s, 1H), 9.32 (s, 1H), 8.68 (dd, J = 8.4, 2.2 Hz, |
| | | 1H), 8.52 (t, J = 8.5 Hz, 1H), 8.38 (s, 1H), 8.20 |
| | | (s, 1H), 7.96 − 7.72 (m, 6H), 7.70 − 7.46 (m, |
| | | 2H), 7.33 (dd, J = 17.1, 8.0 Hz, 2H), 5.62 (p, J = |
| | | 6.1 Hz, 1H), 4.83 − 4.56 (m, 1H), 3.79 − 3.36 |
| | | (m, 1H), 2.99 (d, J = 7.9 Hz, 1H), 2.87 − 2.72 |
| | | (m, 2H), 2.52 (m, 2H), 2.44 − 2.29 (m, 1H), |
| | | 2.25 − 2.09 (m, 3H), 2.02 − 1.91 (m, 1H), 1.83 |
| | | (q, J = 10.1 Hz, 1H), 1.35 (d, J = 6.1 Hz, 6H). |
| 33 | 804.3 | ¹H NMR (400 MHz, dimethyl sulfoxide-d₆) δ |
| | | 13.18 (s, 1H), 10.54 (d, J = 3.1 Hz, 1H), 10.21 |
| | | (s, 1H), 9.31 (s, 1H), 8.51 (td, J = 8.5, 3.4 Hz, |
| | | 1H), 8.37 (s, 1H), 8.19 (s, 1H), 7.93 − 7.74 (m, |
| | | 4H), 7.62 − 7.50 (m, 3H), 7.38 (d, J = 23.0 Hz, |
| | | 1H), 7.05 − 6.78 (m, 1H), 5.61 (p, J = 6.1 Hz, |
| | | 1H), 3.95 (d, J = 3.1 Hz, 3H), 3.90 (td, J = 6.6, |
| | | 4.9 Hz, 2H), 3.84 − 3.40 (m, 1H), 3.01 (d, J = |
| | | 7.9 Hz, 1H), 2.85 (d, J = 7.2 Hz, 1H), 2.75 (q, J = |
| | | 6.2 Hz, 2H), 2.66 − 2.55 (m, 1H), 2.47 − 2.37 |
| | | (m, 1H), 2.32 − 2.21 (m, 1H), 2.22 − 2.13 (m, |
| | | 1H), 1.90 (q, J = 9.8 Hz, 1H), 1.41 − 1.30 (m, |
| | | 6H). |
| 34 | 832.2 | ¹H NMR (400 MHz, dimethyl sulfoxide-d₆) δ |
| | | 13.18 (s, 1H), 10.93 (s, 1H), 10.20 (s, 1H), 9.33 |
| | | (s, 1H), 8.51 (t, J = 8.5 Hz, 1H), 8.29 (d, J = |
| | | 66.9 Hz, 2H), 7.97 − 7.72 (m, 2H), 7.55 − 7.35 |
| | | (m, 2H), 7.26 (d, J = 7.7 Hz, 1H), 7.16 (s, 1H), |
| | | 7.03 − 6.73 (m, 1H), 6.69 − 6.41 (m, 2H), 5.63 |
| | | (p, J = 6.2 Hz, 1H), 5.03 (dd, J = 13.3, 5.1 Hz, |
| | | 1H), 4.94 (q, J = 4.9 Hz, 1H), 4.76 (t, J = 5.1 |
| | | Hz, 1H), 4.29 (dd, J = 16.9, 7.2 Hz, 1H), 4.17 |
| | | (dd, J = 16.9, 7.0 Hz, 1H), 4.00 − 3.80 (m, 2H), |
| | | 3.52 (d, J = 8.2 Hz, 1H), 3.39 (dd, J = 17.5, 9.7 |
| | | Hz, 1H), 3.00 − 2.80 (m, 1H), 2.62 − 2.53 (m, |
| | | 1H), 2.39 − 2.25 (m, 2H), 2.17 − 2.02 (m, 1H), |
| | | 2.04 − 1.86 (m, 1H), 1.36 (d, J = 6.1 Hz, 6H). |
| 35 | 846.3 | ¹H NMR (400 MHz, dimethyl sulfoxide-d₆) δ |
| | | 13.19 (s, 1H), 10.94 (s, 1H), 10.20 (s, 1H), 9.32 |
| | | (s, 1H), 8.50 (t, J = 8.6 Hz, 1H), 8.29 (d, J = |
| | | 48.9 Hz, 2H), 7.86 (dt, J = 9.9, 2.4 Hz, 2H), |
| | | 7.50 (d, J = 8.3 Hz, 1H), 7.43 (t, J = 8.1 Hz, |
| | | 1H), 7.30 − 7.22 (m, 1H), 7.13 (s, 1H), 6.90 (d, |
| | | J = 8.5 Hz, 1H), 6.63 (s, 1H), 6.58 (dd, J = 8.4, |
| | | 2.0 Hz, 1H), 5.63 (hept, J = 6.1 Hz, 1H), 5.04 |
| | | (dd, J = 13.3, 5.1 Hz, 1H), 4.73 (d, J = 7.9 Hz, |
| | | 1H), 4.46 (s, 1H), 4.35 − 4.11 (m, 3H), 3.70 (dd, |
| | | J = 8.0, 4.2 Hz, 1H), 3.56 (d, J = 10.7 Hz, 1H), |
| | | 3.18 (t, J = 10.6 Hz, 1H), 2.90 (ddd, J = 17.9, |
| | | 13.4, 5.4 Hz, 1H), 2.57 (dd, J = 18.7, 4.9 Hz, |
| | | 1H), 2.44 − 2.26 (m, 1H), 2.06 (dd, J = 21.8, |
| | | 12.2 Hz, 2H), 1.99 − 1.90 (m, 1H), 1.84 (s, 2H), |
| | | 1.36 (d, J = 6.1 Hz, 6H). |
| 36 | 882.4 | ¹H NMR (400 MHz, dimethyl sulfoxide-d₆) δ |
| | | 13.18 (s, 1H), 11.06 (s, 1H), 10.25 (s, 1H), 9.32 |
| | | (s, 1H), 8.60 (d, J = 4.1 Hz, OH), 8.26 (d, J = |
| | | 66.4 Hz, 2H), 7.86 − 7.43 (m, 2H), 6.73 (dd, J = |
| | | 8.6, 5.8 Hz, 1H), 6.65 (d, J = 2.2 Hz, 1H), 6.43 − |
| | | 6.19 (m, 1H), 5.61 (td, J = 6.2, 1.6 Hz, 1H), |
| | | 5.18 (dd, J = 12.8, 5.4 Hz, 1H), 3.22 (d, J = 2.4 |
| | | Hz, 3H), 3.16 (d, J = 11.1 Hz, 2H), 3.06 (d, J = |
| | | 10.4 Hz, 1H), 2.96 − 2.80 (m, 1H), 2.69 − 2.57 |
| | | (m, 2H), 2.46 (s, OH), 2.34 (t, J = 6.4 Hz, 1H), |
| | | 1.90 (d, J = 14.8 Hz, 4H), 1.81 − 1.65 (m, 2H), |
| | | 1.56 (d, J = 6.6 Hz, 3H), 1.32 (td, J = 6.0, 3.8 |
| | | Hz, 6H), 1.25 (d, J = 15.2 Hz, 1H). |
| 37 | 882.2 | ¹H NMR (400 MHz, dimethyl sulfoxide-d₆) δ |
| | | 13.19 (s, 1H), 11.04 (s, 1H), 10.21 (s, 1H), 9.24 |
| | | (d, J = 2.5 Hz, 1H), 8.71 − 8.51 (m, 1H), 8.26 |
| | | (d, J = 64.3 Hz, 2H), 7.73 (t, J = 7.6 Hz, 2H), |
| | | 6.89 − 6.61 (m, 2H), 6.46 (d, J = 8.4 Hz, 1H), |
| | | 5.59 (h, J = 6.0 Hz, 1H), 5.18 (dd, J = 12.9, 5.4 |
| | | Hz, 1H), 3.65 − 3.45 (m, 4H), 3.24 (s, 3H), 3.12 |
| | | (q, J = 9.9, 9.5 Hz, 1H), 2.95 (s, 2H), 2.90 − |
| | | 2.73 (m, 2H), 2.72 − 2.64 (m, 1H), 2.64 − 2.55 |
| | | (m, 2H), 2.45 − 2.31 (m, 2H), 1.96 − 1.84 (m, |
| | | 1H), 1.75 − 1.54 (m, 5H), 1.52 − 1.38 (m, 5H), |
| | | 1.34 (dd, J = 6.2, 3.9 Hz, 6H). |
| 38 | 834.2 | ¹H NMR (300 MHz, dimethyl sulfoxide-d₆) δ |
| | | 13.19 (s, 1H), 10.52 (s, 1H), 10.25 (s, 1H), 9.32 |
| | | (s, 1H), 8.54 (t, J = 8.3 Hz, 1H), 8.29 (d, J = |
| | | 46.1 Hz, 2H), 7.95 (d, J = 1.8 Hz, 1H), 7.94 − |
| | | 7.78 (m, 3H), 7.73 − 7.55 (m, 2H), 7.43 (d, J = |
| | | 9.0 Hz, 1H), 6.89 (dd, J = 9.1, 1.9 Hz, 1H), 6.82 |
| | | (d, J = 1.9 Hz, 1H), 5.62 (p, J = 6.1 Hz, 1H), |
| | | 3.88 (d, J = 4.8 Hz, 5H), 3.66 (s, 2H), 3.24 (s, |
| | | 4H), 2.73 (t, J = 6.6 Hz, 2H), 2.55 (d, J = 5.8 |
| | | Hz, 4H), 1.36 (d, J = 6.2 Hz, 6H), 0.84 (q, J = |
| | | 6.2 Hz, 2H). |
| 39 | 848.2 | ¹H NMR (400 MHz, dimethyl sulfoxide-d₆) δ |
| | | 13.19 (s, 1H), 10.50 (s, 1H), 10.22 (s, 1H), 9.31 |
| | | (d, J = 1.3 Hz, 1H), 8.53 (t, J = 8.2 Hz, 1H), |
| | | 8.28 (d, J = 69.9 Hz, 2H), 7.95 (s, 1H), 7.86 (t, |
| | | J = 9.4 Hz, 3H), 7.71 − 7.53 (m, 2H), 7.42 (d, J = |
| | | 8.9 Hz, 1H), 6.88 (d, J = 9.2 Hz, 1H), 6.80 (s, |
| | | 1H), 5.62 (p, J = 6.1 Hz, 1H), 4.10 (d, J = 14.1 |
| | | Hz, 1H), 3.87 (d, J = 4.4 Hz, 5H), 3.59 (d, J = |
| | | 11.5 Hz, 1H), 3.48 (d, J = 11.7 Hz, 1H), 3.42 − |
| | | 3.35 (m, 1H), 2.86 (t, J = 10.5 Hz, 1H), 2.82 − |
| | | 2.64 (m, 4H), 2.63 − 2.55 (m, 1H), 2.37 − 2.22 |
| | | (m, 1H), 1.36 (d, J = 6.1 Hz, 6H), 1.17 (d, J = |
| | | 5.9 Hz, 3H). |
| 40 | 837.4 | ¹H NMR (400 MHz, dimethyl sulfoxide-d₆) δ |
| | | 13.12 (s, 1H), 10.49 (s, 1H), 9.05 (s, 1H), 8.22 |
| | | (s, 3H), 7.75 (s, 1H), 7.73 − 7.57 (m, 3H), 7.41 |
| | | (d, J = 9.0 Hz, 1H), 7.12 (d, J = 7.9 Hz, 1H), |
| | | 6.93 − 6.75 (m, 2H), 5.50 (h, J = 6.1 Hz, 1H), |
| | | 3.86 (d, J = 8.2 Hz, 5H), 3.82 − 3.65 (m, 3H), |
| | | 3.24 (d, J = 5.5 Hz, 4H), 3.16 − 2.91 (m, 4H), |
| | | 2.72 (t, J = 6.6 Hz, 2H), 2.60 (t, J = 4.9 Hz, |
| | | 4H), 2.26 − 2.12 (m, 1H), 1.96 − 1.82 (m, 1H), |
| | | 1.79 − 1.68 (m, 1H), 1.27 (t, J = 5.6 Hz, 6H), |
| | | 0.92 (d, J = 6.8 Hz, 3H). |
| 41 | 848.1 | ¹H NMR (400 MHz, dimethyl sulfoxide-d₆) δ |
| | | 13.19 (s, 1H), 10.51 (s, 1H), 10.24 (s, 1H), 9.31 |
| | | (s, 1H), 8.53 (t, J = 8.2 Hz, 1H), 8.37 (s, 1H), |
| | | 8.20 (s, 1H), 7.98 (t, J = 1.8 Hz, 1H), 7.90 (dt, J = |
| | | 7.5, 1.7 Hz, 1H), 7.86 − 7.79 (m, 2H), 7.70 − |
| | | 7.57 (m, 2H), 7.43 (d, J = 9.1 Hz, 1H), 6.86 (dd, |
| | | J = 9.3, 1.9 Hz, 1H), 6.75 (d, J = 1.9 Hz, 1H), |
| | | 5.61 (hept, J = 6.2 Hz, 1H), 4.11 (d, J = 7.6 Hz, |
| | | 1H), 3.87 (d, J = 8.6 Hz, 5H), 3.74 (d, J = 14.1 |
| | | Hz, 1H), 3.52 (d, J = 14.0 Hz, 1H), 3.37 (d, J = |
| | | 3.3 Hz, 1H), 3.21 − 3.05 (m, 1H), 2.90 (d, J = |
| | | 10.6 Hz, 1H), 2.80 − 2.69 (m, 2H), 2.56 (d, J = |
| | | 10.9 Hz, 1H), 2.31 (ddd, J = 23.8, 11.9, 6.4 Hz, |
| | | 2H), 1.34 (dd, J = 6.1, 1.2 Hz, 6H), 1.00 (d, J = |
| | | 6.3 Hz, 3H). |
| 42 | 876.3 | ¹H NMR (400 MHz, dimethyl sulfoxide-d₆) δ |
| | | 13.19 (s, 1H), 10.51 (s, 1H), 10.23 (s, 1H), 9.31 |
| | | (s, 1H), 8.53 (t, J = 8.5 Hz, 1H), 8.28 (d, J = |
| | | 69.4 Hz, 2H), 7.95 (d, J = 1.9 Hz, 1H), 7.91 − |
| | | 7.78 (m, 3H), 7.69 − 7.57 (m, 2H), 7.43 (d, J = |
| | | 9.0 Hz, 1H), 6.91 − 6.78 (m, 2 H), 5.62 (p, J = |
| | | 6.1 Hz, 1H), 4.16 (d, J = 14.3 Hz, 1H), 3.88 (d, |
| | | J = 1.7 Hz, 5H), 3.56 − 3.36 (m, 3H), 2.90 − |
| | | 2.68 (m, 5H), 2.42 − 2.20 (m, 3H), 1.36 (d, J = |
| | | 6.1 Hz, 6H), 1.02 (d, J = 6.4 Hz, 3H), 0.92 (d, |
| | | J = 6.4 Hz, 3H). |
| 43 | 876.2 | ¹H NMR (300 MHz, dimethyl sulfoxide-d₆) δ |
| | | 13.19 (s, 1H), 10.50 (s, 1H), 10.26 (s, 1H), 9.33 |
| | | (s, 1H), 8.55 (t, J = 8.5 Hz, 1H), 8.29 (d, J = |
| | | 51.7 Hz, 2H), 8.05 − 7.75 (m, 4H), 7.68 − 7.54 |
| | | (m, 2H), 7.36 (d, J = 9.1 Hz, 1H), 6.81 (d, J = |
| | | 9.2 Hz, 1H), 6.69 (s, 1H), 5.63 (p, J = 6.1 Hz, |
| | | 1H), 3.85 (d, J = 11.3 Hz, 5H), 3.74 − 3.52 (m, |
| | | 3H), 3.41 (d, J = 13.7 Hz, 1H), 3.25 (d, J = 11.3 |
| | | Hz, 1H), 2.82 (d, J = 10.7 Hz, 1H), 2.72 (t, J = |
| | | 6.6 Hz, 3H), 2.44 (d, J = 7.0 Hz, 1H), 2.22 (t, |
| | | J = 11.4 Hz, 1H), 2.07 (d, J = 11.6 Hz, 1H), 1.36 |
| | | (d, J = 6.1 Hz, 6H), 0.68 (dd, J = 6.7, 2.0 Hz, |
| | | 6H). |
| 44 | 846.4 | ¹H NMR (400 MHz, dimethyl sulfoxide-d₆) δ |
| | | 13.19 (s, 1H), 10.35 (d, J = 112.6 Hz, 2H), 9.31 |
| | | (s, 1H), 8.51 (t, J = 8.3 Hz, 1H), 8.28 (s, 2H), |
| | | 7.98 (t, J = 1.8 Hz, 1H), 7.94 − 7.74 (m, 3H), |
| | | 7.68 (d, J = 7.6 Hz, 1H), 7.60 (t, J = 7.7 Hz, |
| | | 1H), 7.45 (d, J = 9.0 Hz, 1H), 6.86 (dd, J = 9.1, |
| | | 2.0 Hz, 1H), 6.62 (d, J = 2.0 Hz, 1H), 5.63 (p, |
| | | J = 6.1 Hz, 1H), 3.98 − 3.69 (m, 7H), 3.46 (q, J = |
| | | 6.2, 5.1 Hz, 1H), 3.19 (td, J = 7.6, 6.7, 3.6 Hz, |
| | | 1H), 2.83 − 2.63 (m, 4H), 2.50 − 2.41 (m, 2H), |
| | | 1.36 (d, J = 6.1 Hz, 6H), 0.71 (q, J = 6.0 Hz, |
| | | 1H), 0.31 (q, J = 4.8 Hz, 1H). |
| 45 | 846.1 | ¹H NMR (400 MHz, dimethyl sulfoxide-d₆) δ |
| | | 13.17 (s, 1H), 10.51 (s, 1H), 10.23 (s, 1H), 9.32 |
| | | (s, 1H), 8.51 (t, J = 8.4 Hz, 1H), 8.28 (s, 2H), |
| | | 7.95 (s, 1H), 7.91 − 7.78 (m, 3H), 7.63 (d, J = |
| | | 7.7 Hz, 1H), 7.53 (dd, J = 19.9, 8.4 Hz, 2H), |
| | | 6.85 − 6.75 (m, 1H), 6.59 (s, 1H), 5.62 (p, J = |
| | | 6.1 Hz, 1H), 3.89 (d, J = 16.8 Hz, 6H), 3.78 − |
| | | 3.66 (m, 2H), 3.62 − 3.40 (m, 4H), 2.75 (d, J = |
| | | 6.6 Hz, 2H), 2.50 (s, 2H), 1.62 (d, J = 8.4 Hz, |
| | | 1H), 1.36 (d, J = 6.2 Hz, 6H), 1.23 (s, 1H). |
| 46 | 860.3 | ¹H NMR (300 MHz, dimethyl sulfoxide-d₆) δ |
| | | 13.18 (s, 1H), 10.87 − 10.10 (m, 2H), 9.32 (s, |
| | | 1H), 8.52 (t, J = 8.3 Hz, 1H), 8.29 (d, J = 41.9 |
| | | Hz, 2H), 8.04 (s, 1H), 7.93 − 7.80 (m, 3H), 7.73 |
| | | (d, J = 7.7 Hz, 1H), 7.60 (t, J = 7.7 Hz, 1H), |
| | | 7.42 (d, J = 9.1 Hz, 1H), 6.84 (dd, J = 9.4, 1.9 |
| | | Hz, 1H), 6.67 (s, 1H), 5.62 (p, J = 6.1 Hz, 1H), |
| | | 3.97 − 3.80 (m, 5H), 3.69 (s, 2H), 3.51 (d, J = |
| | | 10.6 Hz, 2H), 3.26 (s, 2H), 2.94 (d, J = 10.5 Hz, |
| | | 2H), 2.73 (t, J = 6.7 Hz, 2H), 2.08 − 1.94 (m, |
| | | 2H), 1.72 (q, J = 6.9, 6.5 Hz, 2H), 1.36 (d, J = |
| | | 6.1 Hz, 6H). |
| 47 | 862.3 | ¹H NMR (400 MHz, dimethyl sulfoxide-d₆) δ |
| | | 13.19 (s, 1H), 10.50 (s, 1H), 10.23 (s, 1H), 9.30 |
| | | (s, 1H), 8.53 (t, J = 8.4 Hz, 1H), 8.36 (s, 1H), |
| | | 8.20 (s, 1H), 7.94 (s, 1H), 7.90 − 7.78 (m, 3H), |
| | | 7.69 (d, J = 7.7 Hz, 1H), 7.59 (t, J = 7.7 Hz, |
| | | 1H), 7.42 (d, J = 9.0 Hz, 1H), 6.87 (d, J = 9.1 |
| | | Hz, 1H), 6.78 (s, 1H), 5.61 (p, J = 6.1 Hz, 1H), |
| | | 3.87 (d, J = 6.8 Hz, 5H), 3.65 (s, 2H), 3.10 (d, J = |
| | | 16.2 Hz, 4H), 2.72 (t, J = 6.6 Hz, 2H), 1.35 |
| | | (d, J = 6.1 Hz, 6H), 1.18 (s, 6H). |
| 48 | 846.2 | ¹H NMR (300 MHz, dimethyl sulfoxide-d₆) δ |
| | | 13.20 (s, 1H), 10.37 (d, J = 82.0 Hz, 2H), 9.33 |
| | | (s, 1H), 8.51 (t, J = 8.3 Hz, 1H), 8.38 (s, 1H), |
| | | 8.20 (s, 1H), 7.95 − 7.75 (m, 4H), 7.67 − 7.47 |
| | | (m, 2H), 7.40 (d, J = 9.0 Hz, 1H), 6.59 (d, J = |
| | | 9.1 Hz, 1H), 6.46 (s, 1H), 5.76 − 5.37 (m, 1H), |
| | | 4.45 (s, 1H), 3.98 − 3.77 (m, 6H), 3.76 − 3.66 |
| | | (m, 1H), 3.60 − 3.51 (m, 2H), 3.52 − 3.44 (m, |
| | | 2H), 2.93 − 2.81 (m, 1H), 2.73 (t, J = 6.7 Hz, |
| | | 2H), 2.03 − 1.80 (m, 2H), 1.36 (d, J = 6.1 Hz, |
| | | 6H). |
| 49 | 851.4 | ¹H NMR (400 MHz, dimethyl sulfoxide-d₆) δ |
| | | 13.09 (s, 1H), 10.50 (d, J = 3.0 Hz, 1H), 9.04 (s, |
| | | 1H), 8.21 (s, 2H), 7.79 (d, J = 3.0 Hz, 1H), 7.76 − |
| | | 7.58 (m, 3H), 7.41 (t, J = 8.9 Hz, 1H), 7.12 |
| | | (dd, J = 7.9, 2.8 Hz, 1H), 6.87 − 6.81 (m, 1H), |
| | | 6.76 (dd, J = 6.8, 1.9 Hz, 1H), 5.50 (p, J = 6.1 |
| | | Hz, 1H), 4.13 (d, J = 12.0 Hz, 1H), 3.86 (dd, J = |
| | | 10.7, 4.2 Hz, 5H), 3.76 (tt, J = 7.5, 4.0 Hz, |
| | | 2H), 3.57 (d, J = 14.0 Hz, 1H), 3.04 (dt, J = |
| | | 60.4, 11.5 Hz, 7H), 2.72 (q, J = 7.0 Hz, 3H), |
| | | 2.40 (d, J = 10.4 Hz, 1H), 2.37 − 2.27 (m, 1H), |
| | | 2.20 (d, J = 9.0 Hz, 1H), 1.88 (t, J = 8.8 Hz, |
| | | 1H), 1.80 − 1.66 (m, 1H), 1.27 (dt, J = 6.6, 3.6 |
| | | Hz, 6H), 1.03 (dd, J = 13.1, 6.4 Hz, 3H), 0.92 |
| | | (t, J = 7.0 Hz, 3H). |
| 50 | 865.3 | ¹H NMR (400 MHz, dimethyl sulfoxide-d₆) δ |
| | | 8.93 (d, J = 4.1 Hz, 1H), 8.26 (s, 2H), 7.84 − |
| | | 7.58 (m, 4H), 7.33 (dd, J = 19.4, 8.9 Hz, 1H), |
| | | 6.98 − 6.65 (m, 2H), 5.41 (dt, J = 12.2, 6.0 Hz, |
| | | 1H), 4.15 − 4.03 (m, 1H), 3.93 − 3.78 (m, 5H), |
| | | 3.71 (s, 1H), 3.49 (t, J = 12.2 Hz, 2H), 3.42 − |
| | | 3.29 (m, 1H), 3.12 (s, 1H), 2.92 (t, J = 10.5 Hz, |
| | | 5H), 2.76 (t, J = 6.7 Hz, 3H), 2.52 − 2.35 (m, |
| | | 3H), 2.21 (s, 1H), 1.98 − 1.82 (m, 1H), 1.82 − |
| | | 1.56 (m, 3H), 1.32 − 1.17 (m, 6H), 1.03 − 0.83 |
| | | (m, 6H). |
| 51 | 851.4 | ¹H NMR (300 MHz, dimethyl sulfoxide-d₆) δ |
| | | 13.09 (s, 1H), 10.51 (s, 1H), 9.05 (d, J = 1.9 Hz, |
| | | 1H), 8.23 (s, 3H), 7.81 − 7.59 (m, 4H), 7.41 (t, J = |
| | | 9.4 Hz, 1H), 7.14 (d, J = 7.8 Hz, 1H), 6.94 − |
| | | 6.72 (m, 2H), 5.51 (p, J = 6.1 Hz, 1H), 4.16 (d, |
| | | J = 14.0 Hz, 1H), 3.87 (d, J = 6.8 Hz, 5H), 3.74 |
| | | (s, 1H), 3.60 (t, J = 8.2 Hz, 1H), 3.45 (d, J = |
| | | 22.1 Hz, 2H), 3.32 (s, 1H), 3.18 − 2.84 (m, 4H), |
| | | 2.73 (t, J = 6.7 Hz, 5H), 2.34 (t, J = 9.7 Hz, |
| | | 1H), 2.20 (s, 1H), 1.88 (s, 1H), 1.75 (s, 1H), |
| | | 1.32 − 1.18 (m, 9H), 0.92 (d, J = 6.9 Hz, 3H). |
| 52 | 865.4 | ¹H NMR (400 MHz, dimethyl sulfoxide-d₆) δ |
| | | 13.11 (s, 1H), 10.50 (s, 1H), 9.05 (s, 1H), 8.22 |
| | | (d, J = 63.5 Hz, 2H), 7.83 − 7.69 (m, 2H), 7.62 |
| | | (d, J = 4.7 Hz, 2H), 7.41 (d, J = 9.0 Hz, 1H), |
| | | 7.13 (d, J = 7.8 Hz, 1H), 6.85 (dd, J = 9.1, 1.9 |
| | | Hz, 1H), 6.79 (d, J = 1.9 Hz, 1H), 5.50 (hept, J = |
| | | 6.1 Hz, 1H), 3.86 (d, J = 10.6 Hz, 5H), 3.63 − |
| | | 2.78 (m, 3H), 3.20 − 2.89 (m, 8H), 2.72 (t, J = |
| | | 6.7 Hz, 2H), 2.56 (t, J = 4.9 Hz, 2H), 2.19 (s, |
| | | 1H), 1.88 (d, J = 6.6 Hz, 1H), 1.74 (dt, J = 9.2, |
| | | 4.4 Hz, 1H), 1.27 (t, J = 5.7 Hz, 6H), 1.21 (s, |
| | | 6H), 0.92 (d, J = 6.9 Hz, 3H). |
| 53 | 851.3 | ¹H NMR (400 MHz, dimethyl sulfoxide-d₆) δ |
| | | 13.18 (s, 1H), 11.07 (s, 1H), 10.46 (s, 1H), 9.33 |
| | | (s, 1H), 8.38 (s, 1H), 8.32 − 8.10 (m, 3H), 7.71 |
| | | (d, J = 8.5 Hz, 1H), 7.24 (d, J = 2.2 Hz, 1H), |
| | | 7.07 (d, J = 1.6 Hz, 1H), 6.98 (d, J = 8.1 Hz, |
| | | 1H), 6.89 (dd, J = 8.3, 1.6 Hz, 1H), 5.66 (hept, |
| | | J = 6.2 Hz, 1H), 5.32 (dd, J = 12.7, 5.3 Hz, 1H), |
| | | 3.32 − 3.31 (m, 2H), 3.03 (t, J = 7.2 Hz, 2H), |
| | | 2.95 − 2.80 (m, 3H), 2.73 (s, 3H), 2.71 − 2.53 |
| | | (m, 3H), 2.27 (t, J = 7.0 Hz, 2H), 2.03 − 1.85 |
| | | (m, 3H), 1.81 − 1.53 (m, 7H), 1.38 (d, J = 6.1 |
| | | Hz, 6H). |
| 54 | 811.3 | ¹H NMR (400 MHz, dimethyl sulfoxide-d₆) δ |
| | | 9.30 (s, 1H), 8.30 (s, 20H), 8.03 − 7.84 (m, 2H), |
| | | 7.81 − 7.51 (m, 2H), 7.07 (d, J = 1.5 Hz, 1H), |
| | | 7.00 (d, J = 8.1 Hz, 1H), 6.91 (dd, J = 8.3, 1.6 |
| | | Hz, 1H), 5.62 (hept, J = 6.1 Hz, 1H), 5.30 (dd, J = |
| | | 12.7, 5.3 Hz, 1H), 3.56 (s, 1H), 3.32 (s, 3H), |
| | | 3.03 − 2.81 (m, 5H), 2.78 − 2.58 (m, 5H), 2.30 |
| | | (t, J = 7.1 Hz, 2H), 2.05 − 1.92 (m, 3H), 1.80 − |
| | | 1.55 (m, 6H), 1.37 (d, J = 6.1 Hz, 6H). |
| 55 | 843.4 | ¹H NMR (400 MHz, dimethyl sulfoxide-d₆) δ |
| | | 13.19 (s, 1H), 11.08 (s, 1H), 10.05 (s, 1H), 9.27 |
| | | (s, 1H), 8.49 − 8.00 (m, 3H), 7.69 (d, J = 8.9 |
| | | Hz, 1H), 7.05 (s, 1H), 6.96 (d, J = 8.1 Hz, 1H), |
| | | 6.87 (d, J = 8.1 Hz, 1H), 5.64 (hept, J = 6.2 Hz, |
| | | 1H), 5.31 (dd, J = 12.9, 5.4 Hz, 1H), 3.31 (s, |
| | | 3H), 3.15 (t, J = 7.2 Hz, 2H), 2.96 − 2.83 (m, |
| | | 3H), 2.80 (s, 3H), 2.75 − 2.56 (m, 2H), 2.51 − |
| | | 2.41 (m, 4H), 2.26 (t, J = 6.9 Hz, 2H), 2.09 − |
| | | 1.85 (m, 3H), 1.79 − 1.55 (m, 6H), 1.36 (d, J = |
| | | 6.1 Hz, 6H). |
| 56 | 847.3 | ¹H NMR (400 MHz, dimethyl sulfoxide-d₆) δ |
| | | 13.19 (s, 1H), 11.08 (s, 1H), 10.46 (s, 1H), 9.33 |
| | | (s, 1H), 8.38 (s, 1H), 8.30 (ddd, J = 9.0, 7.2, 1.6 |
| | | Hz, 1H), 8.21 (s, 1H), 7.63 (ddd, J = 9.2, 7.3, |
| | | 1.9 Hz, 1H), 7.08 (d, J = 1.6 Hz, 1H), 6.99 (d, J = |
| | | 8.1 Hz, 1H), 6.90 (dd, J = 8.2, 1.6 Hz, 1H), |
| | | 5.64 (hept, J = 6.2 Hz, 1H), 5.32 (dd, J = 12.7, |
| | | 5.4 Hz, 1H), 3.32 (s, 3H), 3.14 (t, J = 7.2 Hz, |
| | | 2H), 2.97 − 2.84 (m, 3H), 2.81 (s, 3H), 2.74 − |
| | | 2.61 (m, 2H), 2.62 − 2.52 (m, 1H), 2.30 (t, J = |
| | | 7.1 Hz, 2H), 2.05 − 1.90 (m, 3H), 1.78 − 1.61 |
| | | (m, 6H), 1.37 (d, J = 6.1 Hz, 6H). |
| 57 | 843.3 | ¹H NMR (400 MHz, dimethyl sulfoxide-d₆) δ |
| | | 9.15 (s, 1H), 8.34 (s, 1H), 8.17 (s, 1H), 7.60 − |
| | | 7.30 (m, 2H), 7.08 (s, 1H), 6.99 (d, J = 8.1 Hz, |
| | | 1H), 6.91 (d, J = 8.1 Hz, 1H), 5.54 (p, J = 6.1 |
| | | Hz, 1H), 5.32 (dd, J = 12.9, 5.3 Hz, 1H), 3.32 |
| | | (s, 3H), 3.07 (t, J = 7.1 Hz, 2H), 2.99 − 2.83 (m, |
| | | 3H), 2.80 − 2.56 (m, 6H), 2.39 (s, 3H), 2.32 (t, J = |
| | | 7.1 Hz, 2H), 2.05 − 1.90 (m, 3H), 1.79 − 1.57 |
| | | (m, 6H), 1.34 (d, J = 6.1 Hz, 6H). |
| 58 | 847.3 | ¹H NMR (400 MHz, dimethyl sulfoxide-d₆) δ |
| | | 13.16 (s, 1H), 11.07 (s, 1H), 9.78 (s, 1H), 9.21 |
| | | (s, 1H), 8.34 (s, 1H), 8.18 (s, 1H), 7.64 (d, J = |
| | | 6.8 Hz, 2H), 7.08 (d, J = 1.5 Hz, 1H), 6.99 (d, J = |
| | | 8.1 Hz, 1H), 6.90 (d, J = 8.0 Hz, 1H), 5.53 (p, |
| | | J = 6.2 Hz, 1H), 5.33 (dd, J = 12.7, 5.4 Hz, 1H), |
| | | 3.31 (s, 3H), 3.10 (t, J = 7.2 Hz, 2H), 3.00 − |
| | | 2.84 (m, 3H), 2.79 (s, 3H), 2.76 − 2.52 (m, 2H), |
| | | 2.32 (t, J = 7.1 Hz, 2H), 2.05 − 1.91 (m, 3H), |
| | | 1.69 (dd, J = 16.9, 8.8 Hz, 7H), 1.34 (d, J = 6.1 |
| | | Hz, 6H). |
| 59 | 843.5 | ¹H NMR (400 MHz, dimethyl sulfoxide-d₆) δ |
| | | 13.19 (s, 1H), 11.08 (s, 1H), 10.10 (s, 1H), 9.34 |
| | | (s, 1H), 8.50 − 8.10 (m, 3H), 7.62 (d, J = 11.2 |
| | | Hz, 1H), 7.06 (d, J = 1.6 Hz, 1H), 6.98 (d, J = |
| | | 8.1 Hz, 1H), 6.88 (dd, J = 8.2, 1.6 Hz, 1H), 5.62 |
| | | (hept, J = 6.1 Hz, 1H), 5.33 (dd, J = 12.8, 5.4 |
| | | Hz, 1H), 3.32 (s, 3H), 3.15 (t, J = 7.3 Hz, 2H), |
| | | 2.96 − 2.83 (m, 3H), 2.81 (s, 3H), 2.75 − 2.57 |
| | | (m, 2H), 2.54 (s, 3H), 2.48 − 2.41 (m, 1H), 2.25 |
| | | (t, J = 6.8 Hz, 2H), 2.07 − 1.84 (m, 3H), 1.76 − |
| | | 1.58 (m, 6H), 1.37 (d, J = 6.1 Hz, 6H). |
| 60 | 847.3 | ¹H NMR (400 MHz, dimethyl sulfoxide-d₆) δ |
| | | 13.21 (s, 1H), 11.08 (s, 1H), 10.52 (s, 1H), 9.41 |
| | | (s, 1H), 8.47 (dd, J = 12.8, 6.5 Hz, 1H), 8.30 (d, |
| | | J = 62.0 Hz, 2H), 7.63 (dd, J = 10.4, 6.3 Hz, |
| | | 1H), 7.07 (s, 1H), 7.02 (d, J = 8.1 Hz, 1H), 6.91 |
| | | (d, J = 8.1 Hz, 1H), 5.62 (p, J = 6.1 Hz, 1H), |
| | | 5.34 (dd, J = 12.8, 5.3 Hz, 1H), 3.17 (t, J = 7.0 |
| | | Hz, 2H), 3.11 − 3.02 (m, 1H), 2.96 − 2.85 (m, |
| | | 2H), 2.82 (s, 3H), 2.77 − 2.58 (m, 4H), 2.44 − |
| | | 2.29 (m, 1H), 2.05 − 1.95 (m, 1H), 1.80 (s, 6H), |
| | | 1.38 (d, J = 6.2 Hz, 6H), 1.23 (s, 3H), 1.15 − |
| | | 0.95 (m, 1H), 0.94 − 0.70 (m, 1H). |
| 61 | 862.3 | ¹H NMR (400 MHz, dimethyl sulfoxide-d₆) δ |
| | | 13.14 (s, 1H), 10.96 (s, 1H), 10.49 (s, 1H), 9.86 |
| | | (s, 1H), 9.25 (s, 1H), 8.73 − 8.40 (m, 3H), 8.31 − |
| | | 7.91 (m, 3H), 6.98 (s, 1H), 6.91 − 6.67 (m, |
| | | 2H), 5.61 (p, J = 6.1 Hz, 1H), 5.22 (dd, J = |
| | | 12.9, 5.3 Hz, 1H), 3.23 (s, 3H), 3.13 (t, J = 7.2 |
| | | Hz, 2H), 2.80 (s, 4H), 2.68 (d, J = 10.5 Hz, 2H), |
| | | 2.62 − 2.47 (m, 2H), 2.41 − 2.28 (m, 1H), 2.10 |
| | | (t, J = 7.0 Hz, 2H), 1.96 − 1.85 (m, 1H), 1.75 (t, |
| | | J = 11.1 Hz, 2H), 1.65 − 1.44 (m, 6H), 1.30 (d, |
| | | J = 6.1 Hz, 6H). |
| 62 | 844.3 | ¹H NMR (400 MHz, dimethyl sulfoxide-d₆) δ |
| | | 13.19 (s, 1H), 11.06 (s, 1H), 10.16 (s, 1H), 9.34 |
| | | (s, 1H), 8.53 (t, J = 8.5 Hz, 1H), 8.29 (d, J = |
| | | 62.5 Hz, 2H), 7.72 − 7.28 (m, 2H), 6.92 (d, J = |
| | | 8.5 Hz, 1H), 6.82 (d, J = 2.2 Hz, 1H), 6.60 (dd, |
| | | J = 8.6, 2.2 Hz, 1H), 5.64 (hept, J = 6.2 Hz, |
| | | 1H), 5.28 (dd, J = 12.8, 5.4 Hz, 1H), 3.40 − |
| | | 3.33 (m, 1H), 3.30 (s, 3H), 3.11 − 2.90 (m, 2H), |
| | | 2.92 − 2.82 (m, 2H), 2.82 − 2.66 (m, 6H), 2.68 − |
| | | 2.56 (m, 2H), 2.51 − 2.43 (m, 2H), 2.32 (t, J = |
| | | 10.1 Hz, 1H), 2.28 − 2.13 (m, 1H), 2.05 − 1.88 |
| | | (m, 1H), 1.72 − 1.56 (m, 2H), 1.37 (d, J = 6.1 |
| | | Hz, 6H), 1.06 (d, J = 4.9 Hz, 3H). |
| 63 | 844.3 | ¹H NMR (400 MHz, dimethyl sulfoxide-d₆) δ |
| | | 13.19 (s, 1H), 11.06 (s, 1H), 10.16 (s, 1H), 9.34 |
| | | (s, 1H), 8.53 (t, J = 8.5 Hz, 1H), 8.26 (d, J = |
| | | 37.0 Hz, 2H), 7.81 − 7.46 (m, 2H), 6.92 (d, J = |
| | | 8.6 Hz, 1H), 6.82 (d, J = 2.2 Hz, 1H), 6.60 (dd, |
| | | J = 8.7, 2.2 Hz, 1H), 5.64 (hept, J = 6.1 Hz, |
| | | 1H), 5.28 (dd, J = 12.9, 5.3 Hz, 1H), 3.30 (s, |
| | | 3H), 3.00 (ddp, J =20.6, 14.1, 6.8 Hz, 3H), 2.87 |
| | | (dt, J = 12.7, 4.8 Hz, 2H), 2.77 (t, J = 10.6 Hz, |
| | | 2H), 2.73 − 2.55 (m, 7H), 2.47 (s, 1H), 2.38 − |
| | | 2.27 (m, 1H), 2.22 (dt, J = 13.1, 6.9 Hz, 1H), |
| | | 2.05 − 1.91 (m, 1H), 1.64 (dq, J = 14.2, 7.1 Hz, |
| | | 2H), 1.37 (d, J = 6.1 Hz, 6H), 1.06 (d, J = 5.0 |
| | | Hz, 3H). |
| 64 | 872.3 | ¹H NMR (400 MHz, dimethyl sulfoxide-d₆) δ |
| | | 9.29 (s, 1H), 8.55 (t, J = 8.5 Hz, 1H), 8.30 (d, J = |
| | | 55.7 Hz, 2H), 7.73 − 7.49 (m, 2H), 6.94 (d, J = |
| | | 8.6 Hz, 1H), 6.80 (d, J = 2.2 Hz, 1H), 6.63 |
| | | (dd, J = 8.6, 2.2 Hz, 1H), 5.63 (hept, J = 6.2 Hz, |
| | | 1H), 5.25 (dd, J = 12.7, 5.4 Hz, 1H), 3.31 (s, |
| | | 5H), 3.11 −2.82 (m, 4H), 2.79 −2.69 (m, 7H), |
| | | 2.69 − 2.56 (m, 1H), 2.42 (t, J = 10.3 Hz, 1H), |
| | | 2.31 (dt, J = 13.3, 7.2 Hz, 1H), 2.23 (s, 1H), |
| | | 2.13 (p, J = 6.3 Hz, 1H), 2.05 − 1.94 (m, 1H), |
| | | 1.68 − 1.58 (m, 2H), 1.37 (d, J = 6.2 Hz, 6H), |
| | | 0.97 (d, J = 6.8 Hz, 3H), 0.87 (d, J = 6.7 Hz, |
| | | 3H). |
| 65 | 872.3 | ¹H NMR (400 MHz, dimethyl sulfoxide-d₆) δ |
| | | 13.18 (s, 1H), 11.06 (s, 1H), 10.16 (s, 1H), 9.34 |
| | | (s, 1H), 8.54 (t, J = 8.5 Hz, 1H), 8.27 (d, J = |
| | | 43.3 Hz, 2H), 7.69 − 7.55 (m, 2H), 6.92 (d, J = |
| | | 8.5 Hz, 1H), 6.80 (d, J = 2.2 Hz, 1H), 6.61 (dd, |
| | | J = 8.6, 2.2 Hz, 1H), 5.65 (p, J = 6.2 Hz, 1H), |
| | | 5.28 (dd, J = 12.9, 5.4 Hz, 1H), 3.26 (s, 4H), |
| | | 3.08 (dt, J = 14.1, 7.2 Hz, 1H), 3.01 − 2.96 (m, |
| | | 1H), 2.96 − 2.82 (m, 3H), 2.81 − 2.61 (m, 7H), |
| | | 2.61 − 2.53 (m, 1H), 2.41 (t, J = 10.5 Hz, 1H), |
| | | 2.30 (dt, J = 13.3, 6.7 Hz, 1H), 2.17 (dq, J = |
| | | 32.1, 6.2 Hz, 2H), 2.05 − 1.92 (m, 1H), 1.69 − |
| | | 1.57 (m, 2H), 1.37 (d, J = 6.2 Hz, 6H), 0.97 (d, |
| | | J = 6.7 Hz, 3H), 0.88 (d, J = 6.7 Hz, 3H). |
| 66 | 830.1 | ¹H NMR (400 MHz, dimethyl sulfoxide-d₆) δ |
| | | 13.18 (s, 1H), 11.05 (s, 1H), 10.15 (s, 1H), 9.33 |
| | | (s, 1H), 8.53 (t, J = 8.5 Hz, 1H), 8.38 (s, 1H), |
| | | 8.20 (s, 1H), 7.71 − 7.55 (m, 2H), 6.92 (d, J = |
| | | 8.6 Hz, 1H), 6.83 (d, J = 2.2 Hz, 1H), 6.61 (dd, |
| | | J = 8.6, 2.2 Hz, 1H), 5.64 (p, J = 6.1 Hz, 1H), |
| | | 5.28 (dd, J = 12.9, 5.3 Hz, 1H), 3.30 (s, 4H), |
| | | 3.08 (t, J = 4.8 Hz, 4H), 3.02 (t, J = 7.1 Hz, |
| | | 2H), 2.94 − 2.83 (m, 2H), 2.72 (s, 4H), 2.70 − |
| | | 2.56 (m, 2H), 2.35 (t, J = 7.1 Hz, 2H), 2.04 − |
| | | 1.92 (m, 1H), 1.73 − 1.61 (m, 2H), 1.37 (d, J = |
| | | 6.1 Hz, 6H). |
| 67 | 858.3 | ¹H NMR (400 MHz, dimethyl sulfoxide-d₆) δ |
| | | 13.19 (s, 1H), 11.06 (s, 1H), 10.16 (s, 1H), 9.33 |
| | | (s, 1H), 8.53 (t, J = 8.5 Hz, 1H), 8.29 (s, 2H), |
| | | 7.70 − 7.56 (m, 2H), 6.91 (d, J = 8.6 Hz, 1H), |
| | | 6.80 (d, J = 2.2 Hz, 1H), 6.59 (dd, J = 8.7, 2.2 |
| | | Hz, 1H), 5.64 (hept, J = 6.1 Hz, 1H), 5.28 (dd, |
| | | J = 12.9, 5.4 Hz, 1H), 3.30 (s, 3H), 3.02 (q, J = |
| | | 7.9, 6.6 Hz, 4H), 2.89 (ddd, J = 16.6, 13.4, 5.2 |
| | | Hz, 1H), 2.79 (s, 2H), 2.73 − 2.55 (m, 7H), 2.38 − |
| | | 2.29 (m, 2H), 2.04 − 1.92 (m, 1H), 1.63 − 1.51 |
| | | (m, 2H), 1.37 (d, J = 6.2 Hz, 6H), 1.04 (s, 6H). |
| 68 | 856.2 | ¹H NMR (400 MHz, dimethyl sulfoxide-d₆) δ |
| | | 13.45 − 13.02 (m, 1H), 11.05 (s, 1H), 10.15 (s, |
| | | 1H), 9.33 (d, J = 1.5 Hz, 1H), 8.52 (t, J = 8.5 |
| | | Hz, 1H), 8.38 (s, 1H), 8.20 (s, 1H), 7.63 (ddd, J = |
| | | 10.9, 7.8, 2.2 Hz, 2H), 6.88 (d, J = 8.4 Hz, |
| | | 1H), 6.68 (d, J = 2.2 Hz, 1H), 6.47 (dd, J = 8.8, |
| | | 2.3 Hz, 1H), 5.64 (p, J = 6.2 Hz, 1H), 5.26 (dd, |
| | | J = 12.8, 5.2 Hz, 1H), 3.31 − 3.23 (m, 5H), 3.06 |
| | | (t, J = 7.1 Hz, 2H), 2.87 (d, J = 17.5 Hz, 1H), |
| | | 2.78 (d, J = 10.0 Hz, 2H), 2.72 (s, 3H), 2.63 (td, |
| | | J = 16.8, 8.2 Hz, 2H), 2.52 − 2.51 (m, 2H), 2.39 − |
| | | 2.28 (m, 2H), 2.02 − 1.92 (m, 1H), 1.86 (s, |
| | | 2H), 1.72 − 1.57 (m, 4H), 1.37 (dd, J = 6.1, 1.5 |
| | | Hz, 6H). |
| 69 | 842.3 | ¹H NMR (400 MHz, dimethyl sulfoxide-d₆) δ |
| | | 13.19 (s, 1H), 11.05 (s, 1H), 10.15 (s, 1H), 9.34 |
| | | (s, 1H), 8.51 (t, J = 8.5 Hz, 1H), 8.38 − 8.20 (m, |
| | | 2H), 7.65 − 7.57 (m, 2H), 6.87 (d, J = 8.5 Hz, |
| | | 1H), 6.43 (s, 1H), 6.24 (d, J = 7.5 Hz, 1H), 5.64 |
| | | (p, J = 6.2 Hz, 1H), 5.25 (dd, J = 12.9, 5.4 Hz, |
| | | 1H), 4.27 (s, 1H), 3.49 (s, 1H), 3.28 (s, 3H), |
| | | 3.11 (d, J = 9.0 Hz, 1H), 3.00 − 2.81 (m, 4H), |
| | | 2.69 − 2.56 (m, 5H), 2.48 − 2.38 (m, 4H), 1.98 |
| | | (d, J = 11.9 Hz, 1H), 1.79 (dd, J = 8.9 Hz, 2H), |
| | | 1.56 − 1.48 (m, 2H), 1.37 (d, J = 6.1 Hz, 6H) |
| 70 | 842.1 | ¹H NMR (400 MHz, dimethyl sulfoxide-d₆) δ |
| | | 13.19 (s, 1H), 11.05 (s, 1H), 10.15 (s, 1H), 9.34 |
| | | (s, 1H), 8.54 − 8.18 (m, 3H), 7.65 − 7.57 (m, |
| | | 2H), 6.95 (d, J = 8.6 Hz, 1H), 6.57 − 6.51 (m, |
| | | 1H), 6.43 − 6.36 (m, 1H), 5.64 (p, J = 6.2 Hz, |
| | | 1H), 5.28 (dd, J = 12.8, 5.3 Hz, 1H), 3.66 (s, |
| | | 2H), 3.42 (d, J = 10.9 Hz, 2H), 3.32 − 3.23 (m, |
| | | 5H), 3.01 − 2.85 (m, 3H), 2.74 − 2.57 (m, 5H), |
| | | 2.43 (s, 1H), 2.25 (s, 2H), 2.02 − 1.94 (m, 1H), |
| | | 1.52 (s, 3H), 1.37 (d, J = 6.1 Hz, 6H) |
| 71 | 841.2 | ¹H NMR (400 MHz, dimethyl sulfoxide-d₆) δ |
| | | 13.17 (s, 1H), 11.08 (s, 1H), 10.17 (d, J = 2.0 |
| | | Hz, 1H), 9.32 (s, 1H), 8.55 (t, J = 8.5 Hz, 1H), |
| | | 8.29 (d, J = 46.0 Hz, 2H), 7.80 − 7.63 (m, 2H), |
| | | 7.08 (d, J = 1.5 Hz, 1H), 6.98 (d, J = 8.1 Hz, |
| | | 1H), 6.90 − 6.80 (m, 1H), 5.63 (p, J = 6.1 Hz, |
| | | 1H), 5.32 (dd, J = 12.8, 5.3 Hz, 1H), 3.31 − |
| | | 3.14 (m, 3H), 2.98 (dd, J = 10.2, 5.9 Hz, 1H), |
| | | 2.87 (d, J = 17.3 Hz, 2H), 2.78 − 2.54 (m, 5H), |
| | | 2.45 (s, 1H), 2.38 − 2.24 (m, 1H), 2.16 − 1.80 |
| | | (m, 6H), 1.67 (s, 4H), 1.47 (dp, J = 14.0, 7.5, |
| | | 6.7 Hz, 2H), 1.36 (d, J = 6.1 Hz, 6H). |
| 72 | 855.4 | ¹H NMR (400 MHz, dimethyl sulfoxide-d₆) δ |
| | | 13.19 (s, 1H), 11.09 (s, 1H), 10.18 (s, 1H), 9.33 |
| | | (s, 1H), 8.55 (t, J = 8.3 Hz, 1H), 8.29 (d, J = |
| | | 67.1 Hz, 2H), 7.83 − 7.46 (m, 2H), 7.11 (d, J = |
| | | 1.6 Hz, 1H), 7.01 (d, J = 8.1 Hz, 1H), 6.92 (dd, |
| | | J = 8.1, 1.5 Hz, 1H), 5.65 (hept, J = 6.1 Hz, |
| | | 1H), 5.34 (dd, J = 12.7, 5.4 Hz, 1H), 3.60 (d, J = |
| | | 10.9 Hz, 3H), 3.51 (d, J = 11.1 Hz, 3H), 3.04 − |
| | | 2.82 (m, 3H), 2.77 − 2.53 (m, 3H), 2.40 (t, J = |
| | | 11.1 Hz, 1H), 2.14 (q, J = 11.9, 10.5 Hz, 3H), |
| | | 2.04 − 1.87 (m, 3H), 1.84 − 1.60 (m, 7H), 1.49 |
| | | (d, J = 12.5 Hz, 1H), 1.37 (d, J = 6.1 Hz, 6H). |
| 73 | 838.4 | ¹H NMR (400 MHz, dimethyl sulfoxide-d₆) δ |
| | | 13.18 (s, 1H), 11.06 (s, 1H), 10.30 (s, 1H), 9.27 |
| | | (s, 1H), 8.58 (t, J = 8.4 Hz, 1H), 8.36 (s, 1H), |
| | | 8.19 (s, 1H), 7.80 (ddd, J = 21.4, 9.5, 2.2 Hz, |
| | | 2H), 7.36 (d, J = 3.5 Hz, 1H), 7.03 (s, 1H), 6.95 − |
| | | 6.74 (m, 2H), 6.59 (d, J = 3.5 Hz, 1H), 5.61 |
| | | (p, J = 6.1 Hz, 1H), 5.28 (dd, J = 13.0, 5.3 Hz, |
| | | 1H), 3.60 (s, 2H), 3.28 (s, 3H), 2.85 (d, J = 10.9 |
| | | Hz, 3H), 2.72 − 2.53 (m, 3H), 2.43 − 2.27 (m, |
| | | 1H), 2.06 − 1.88 (m, 3H), 1.73 − 1.58 (m, 3H), |
| | | 1.35 (d, J = 6.1 Hz, 6H). |
| 74 | 868.2 | ¹H NMR (400 MHz, dimethyl sulfoxide-d₆) δ |
| | | 13.19 (s, 1H), 11.07 (s, 1H), 10.23 (s, 1H), 9.36 |
| | | (s, 1H), 8.60 (t, J = 8.5 Hz, 1H), 8.39 (s, 1H), |
| | | 8.21 (s, 1H), 7.77 − 7.65 (m, 2H), 6.94 (t, J = |
| | | 7.9 Hz, 1H), 6.88 − 6.80 (m, 2H), 5.66 (p, J = |
| | | 6.1 Hz, 1H), 5.33 (dd, J = 12.7, 5.4 Hz, 1H), |
| | | 4.59 (t, J = 5.5 Hz, 1H), 3.81 (dd, J = 9.4, 3.1 |
| | | Hz, 1H), 3.71 − 3.61 (m, 1H), 3.58 (s, 3H), 3.43 |
| | | (dd, J = 9.4, 6.8 Hz, 1H), 3.11 − 2.95 (m, 3H), |
| | | 2.93 − 2.76 (m, 2H), 2.74 − 2.54 (m, 4H), 2.44 |
| | | (s, 2H), 2.01 − 1.94 (m, 1H), 1.84 (dd, J = 13.0, |
| | | 5.0 Hz, 1H), 1.74 (s, 3H), 1.46 (s, 1H), 1.38 (d, |
| | | J = 6.1 Hz, 6H), 1.26 (s, 2H). |
| 75 | 868.4 | ¹H NMR (400 MHz, dimethyl sulfoxide-d₆) δ |
| | | 13.19 (s, 1H), 11.06 (s, 1H), 10.21 (s, 1H), 9.28 |
| | | (d, J = 1.1 Hz, 1H), 8.59 (t, J = 8.5 Hz, 1H), |
| | | 8.28 (s, 2H), 7.70 (t, J = 8.0 Hz, 2H), 6.80 (t, J = |
| | | 8.0 Hz, 1H), 6.60 (d, J = 7.8 Hz, 1H), 6.46 (d, |
| | | J = 8.2 Hz, 1H), 5.61 (t, J = 6.2 Hz, 1H), 5.25 |
| | | (s, 1H), 3.87 − 3.69 (m, 2H), 3.50 (s, 3H), 3.24 − |
| | | 3.02 (m, 4H), 2.83 (d, J = 15.4 Hz, 1H), 2.68 − |
| | | 2.53 (m, 5H), 2.36 (d, J = 8.1 Hz, 2H), 2.00 |
| | | (s, 2H), 1.91 (s, 1H), 1.74 (d, J = 7.0 Hz, 2H), |
| | | 1.53 (s, 3H), 1.33 (dd, J = 5.5, 3.0 Hz, 7H). |
| 76 | 896.5 | ¹H NMR (400 MHz, dimethyl sulfoxide-d₆) δ |
| | | 13.19 (s, 1H), 11.06 (s, 1H), 10.31 (s, 1H), 9.32 |
| | | (s, 1H), 8.62 (t, J = 8.2 Hz, 1H), 8.29 (d, J = |
| | | 67.5 Hz, 2H), 7.73 (d, J = 9.4 Hz, 2H), 6.93 − |
| | | 6.48 (m, 3H), 5.60 (p, J = 6.1 Hz, 1H), 5.39 − |
| | | 5.19 (m, 1H), 3.52 (d, J = 25.8 Hz, 5H), 3.43 (d, |
| | | J = 8.7 Hz, 2H), 3.00 (d, J = 10.8 Hz, 2H), 2.83 |
| | | (d, J = 15.4 Hz, 1H), 2.69 − 2.54 (m, 4H), 2.16 |
| | | (s, 2H), 2.11 − 1.82 (m, 5H), 1.62 (d, J = 12.1 |
| | | Hz, 2H), 1.47 − 1.26 (m, 11H), 1.25 − 1.09 (m, |
| | | 2H). |
| 77 | 819.4 | ¹H NMR (400 MHz, dimethyl sulfoxide-d₆) δ |
| | | 13.20 (s, 1H), 10.23 (s, 1H), 9.32 (d, J = 1.0 Hz, |
| | | 1H), 8.53 (t, J = 8.4 Hz, 1H), 8.29 (d, J = 68.2 |
| | | Hz, 2H), 7.92 (s, 1H), 7.86 (dd, J = 10.2, 7.8 |
| | | Hz, 3H), 7.69 (d, J = 7.8 Hz, 1H), 7.65 − 7.53 |
| | | (m, 3H), 7.50 (d, J = 7.9 Hz, 1H), 5.62 (hept, |
| | | J = 6.1 Hz, 1H), 5.17 (dd, J = 13.4, 5.1 Hz, 1H), |
| | | 4.43 (d, J = 17.2 Hz, 1H), 4.30 (d, J = 17.2 Hz, |
| | | 1H), 3.87 − 3.77 (m, 1H), 3.75 (s, 2H), 3.68 (t, |
| | | J = 7.2 Hz, 2H), 3.19 (dt, J = 6.8, 3.9 Hz, 2H), |
| | | 3.00 (s, 3H), 2.95 (d, J = 5.4 Hz, 1H), 2.83 − |
| | | 2.70 (m, 1H), 2.39 (qd, J = 13.3, 4.7 Hz, 1H), |
| | | 2.06 − 1.95 (m, 1H), 1.35 (d, J = 6.1 Hz, 6H). |
| 78 | 805.3 | ¹H NMR (400 MHz, dimethyl sulfoxide-d₆) δ |
| | | 13.18 (s, 1H), 10.55 (s, 1H), 10.22 (d, J = 2.0 |
| | | Hz, 1H), 9.31 (s, 1H), 8.51 (t, J = 8.5 Hz, 1H), |
| | | 8.37 (s, 1H), 8.19 (s, 1H), 7.93 (d, J = 1.8 Hz, |
| | | 1H), 7.91 − 7.77 (m, 3H), 7.71 − 7.54 (m, 3H), |
| | | 7.50 (s, 1H), 7.12 (dd, J = 8.6, 1.3 Hz, 1H), 5.62 |
| | | (p, J = 6.1 Hz, 1H), 3.97 (s, 3H), 3.91 (t, J = 6.6 |
| | | Hz, 2H), 3.82 (p, J = 7.4 Hz, 1H), 3.76 (s, 2H), |
| | | 3.70 (t, J = 7.2 Hz, 2H), 3.24 (t, J = 6.9 Hz, |
| | | 2H), 2.75 (t, J = 6.7 Hz, 2H), 1.35 (d, J = 6.2 |
| | | Hz, 6H). |
| 79 | 804.3 | ¹H NMR (400 MHz, dimethyl sulfoxide-d₆) δ |
| | | 13.19 (s, 1H), 10.98 (s, 1H), 10.21 (s, 1H), 9.32 |
| | | (s, 1H), 8.51 (t, J = 8.6 Hz, 1H), 8.40 − 8.21 (d, |
| | | J = 69.07 Hz, 2H), 7.83 (ddt, J = 12.7, 9.2, 4.5 |
| | | Hz, 4H), 7.63 (d, J = 7.8 Hz, 1H), 7.53 (dd, J = |
| | | 4.7, 2.2 Hz, 2H), 7.42 (s, 1H), 7.34 (d, J = 7.9 |
| | | Hz, 1H), 5.61 (h, J = 6.2 Hz, 1H), 5.09 (dd, J = |
| | | 13.3, 5.1 Hz, 1H), 4.40 (d, J = 17.2 Hz, 1H), |
| | | 4.28 (d, J = 17.2 Hz, 1H), 3.48 − 3.39 (m, 1H), |
| | | 2.96 − 2.79 (m, 3H), 2.64 − 2.52 (m, 2H), 2.44 − |
| | | 2.31 (m, 3H), 2.03 − 1.94 (m, 1H), 1.85 (q, J = |
| | | 10.1 Hz, 2H), 1.35 (d, J = 6.1 Hz, 6H). |
| 80 | 804.3 | ¹H NMR (400 MHz, dimethyl sulfoxide-d₆) δ |
| | | 13.19 (s, 1H), 11.11 − 10.85 (m, 1H), 10.21 (s, |
| | | 1H), 9.32 (d, J = 1.5 Hz, 1H), 8.51 (t, J = 8.5 |
| | | Hz, 1H), 8.28 (d, J = 69.4 Hz, 2H), 7.94 − 7.76 |
| | | (m, 4H), 7.63 (d, J = 7.8 Hz, 1H), 7.56 (d, J = |
| | | 6.8 Hz, 2H), 7.48 (s, 1H), 7.37 (d, J = 7.9 Hz, |
| | | 1H), 5.62 (p, J = 6.2 Hz, 1H), 5.09 (dd, J = |
| | | 13.3, 5.1 Hz, 1H), 4.40 (d, J = 17.2 Hz, 1H), |
| | | 4.28 (d, J = 17.2 Hz, 1H), 3.77 (p, J = 8.1 Hz, |
| | | 1H), 2.99 (d, J = 7.8 Hz, 2H), 2.90 (ddd, J = |
| | | 17.9, 13.5, 5.5 Hz, 1H), 2.59 (d, J = 15.6 Hz, |
| | | 2H), 2.45 − 2.31 (m, 1H), 2.21 (p, J = 8.7, 7.0 |
| | | Hz, 4H), 1.98 (d, J = 12.6 Hz, 1H), 1.35 (d, J = |
| | | 6.1 Hz, 6H). |
| 81 | 804.3 | ¹H NMR (400 MHz, dimethyl sulfoxide-d₆) δ |
| | | 13.18 (s, 1H), 10.98 (s, 1H), 10.21 (s, 1H), 9.32 |
| | | (s, 1H), 8.51 (t, J = 8.5 Hz, 1H), 8.28 (d, J = |
| | | 70.3 Hz, 2H), 7.83 (ddt, J = 12.5, 9.1, 4.3 Hz, |
| | | 4H), 7.63 (d, J = 7.8 Hz, 1H), 7.59 − 7.50 (m, |
| | | 2H), 7.42 (s, 1H), 7.34 (d, J = 7.9 Hz, 1H), 5.62 |
| | | (p, J = 6.1 Hz, 1H), 5.09 (dd, J = 13.3, 5.1 Hz, |
| | | 1H), 4.40 (d, J = 17.2 Hz, 1H), 4.28 (d, J = 17.1 |
| | | Hz, 1H), 3.49 − 3.39 (m, 1H), 2.91 (ddd, J = |
| | | 17.9, 13.4, 5.4 Hz, 1H), 2.83 (d, J = 7.3 Hz, |
| | | 2H), 2.68 − 2.53 (m, 2H), 2.46 − 2.31 (m, 3H), |
| | | 2.04 − 1.94 (m, 1H), 1.85 (q, J = 10.1 Hz, 2H), |
| | | 1.35 (d, J = 6.1 Hz, 6H). |
| 82 | 804.3 | ¹H NMR (400 MHz, dimethyl sulfoxide-d₆) δ |
| | | 13.19 (s, 1H), 10.97 (s, 1H), 10.22 (s, 1H), 9.32 |
| | | (s, 1H), 8.52 (t, J = 8.5 Hz, 1H), 8.29 (d, J = |
| | | 69.7 Hz, 2H), 7.98 − 7.78 (m, 4H), 7.63 (d, J = |
| | | 7.8 Hz, 1H), 7.56 (d, J = 6.8 Hz, 2H), 7.48 (s, |
| | | 1H), 7.37 (d, J = 7.9 Hz, 1H), 5.62 (p, J = 6.1 |
| | | Hz, 1H), 5.09 (dd, J = 13.3, 5.1 Hz, 1H), 4.40 |
| | | (d, J = 17.3 Hz, 1H), 4.28 (d, J = 17.2 Hz, 1H), |
| | | 3.77 (p, J = 8.3 Hz, 1H), 2.99 (d, J = 7.8 Hz, |
| | | 2H), 2.89 (td, J = 13.2, 6.7 Hz, 1H), 2.64 − 2.54 |
| | | (m, 2H), 2.45 − 2.27 (m, 1H), 2.27 − 2.05 (m, |
| | | 4H), 2.03 − 1.89 (m, 1H), 1.35 (d, J = 6.2 Hz, |
| | | 6H). |
| 83 | 960.2 | ¹H NMR (400 MHz, dimethyl sulfoxide-d₆) δ |
| | | 13.16 (s, 1H), 10.49 (s, 1H), 10.25 (s, 1H), 9.30 |
| | | (d, J = 2.3 Hz, 1H), 8.50 (t, J = 8.4 Hz, 1H), |
| | | 8.28 (s, 2H), 7.96 (d, J = 2.4 Hz, 1H), 7.85 (dd, |
| | | J = 19.2, 8.5 Hz, 3H), 7.69 (t, J = 6.3 Hz, 1H), |
| | | 7.59 (t, J = 7.7 Hz, 1H), 7.44 (t, J = 9.7 Hz, |
| | | 1H), 6.85 (dd, J = 19.1, 9.2 Hz, 1H), 6.60 − |
| | | 6.53 (m, 1H), 5.63 (hept, J = 5.6 Hz, 1H), 3.94 − |
| | | 3.77 (m, 6H), 3.27 − 3.11 (m, 2H), 2.72 (td, |
| | | J = 6.7, 2.9 Hz, 2H), 2.66 (s, 2H), 2.50 − 2.35 (m, |
| | | 2H), 1.43 (dd, J = 10.6, 6.5 Hz, 3H), 1.36 (d, |
| | | J = 6.3 Hz, 6H), 0.91 − 0.64 (m, 1H), 0.24 (dd, |
| | | J = 32.5, 4.7 Hz, 1H). |
| 84 | 848.4 | ¹H NMR (400 MHz, dimethyl sulfoxide-d₆) δ |
| | | 13.18 (s, 1H), 10.48 (s, 1H), 10.22 (s, 1H), 9.30 |
| | | (s, 1H), 8.52 (t, J = 8.2 Hz, 1H), 8.27 (s, 2H), |
| | | 7.95 (t, J = 1.8 Hz, 1H), 7.86 (t, J = 8.5 Hz, |
| | | 3H), 7.67 (d, J = 8.0 Hz, 1H), 7.60 (t, J = 7.7 |
| | | Hz, 1H), 7.39 (d, J = 9.0 Hz, 1H), 6.84 (dd, J = |
| | | 9.1, 1.9 Hz, 1H), 6.77 (d, J = 1.9 Hz, 1H), 5.61 |
| | | (p, J = 6.2 Hz, 1H), 3.94 − 3.78 (m, 5H), 3.65 |
| | | (q, J = 6.6 Hz, 1H), 3.19 (t, J = 4.8 Hz, 4H), |
| | | 2.71 (t, J = 6.7 Hz, 2H), 2.61 − 2.53 (m, 2H), |
| | | 2.47 − 2.37 (m, 2H), 1.35 (d, J = 6.3 Hz, 9H). |
| 85 | 847.3 | ¹H NMR (400 MHz, dimethyl sulfoxide-d₆) δ |
| | | 13.18 (s, 1H), 10.54 (s, 1H), 10.23 (s, 1H), 9.26 |
| | | (s, 1H), 8.59 − 8.45 (m, 1H), 8.38 (br s, 1H), |
| | | 8.15 (br s, 1H), 7.94 (t, J = 1.8 Hz, 1H), 7.86 |
| | | (ddt, J = 10.2, 5.6, 2.3 Hz, 3H), 7.71 − 7.55 (m, |
| | | 2H), 7.49 (d, J = 8.4 Hz, 1H), 7.41 (s, 1H), 6.98 |
| | | (d, J = 8.6 Hz, 1H), 5.60 (p, J = 6.2 Hz, 1H), |
| | | 3.95 (s, 3H), 3.88 (t, J = 6.7 Hz, 2H), 3.69 (d, |
| | | J = 6.9 Hz, 1H), 3.04 (d, J = 10.6 Hz, 1H), 2.82 |
| | | (d, J = 10.7 Hz, 1H), 2.73 (t, J = 6.7 Hz, 2H), |
| | | 2.59 − 2.51 (m, 1H), 2.02 (t, J = 10.5 Hz, 1H), |
| | | 1.98 − 1.86 (m, 1H), 1.86 − 1.66 (m, 4H), 1.34 |
| | | (t, J = 5.9 Hz, 9H). |
| 86 | 812.2 | ¹H NMR (400 MHz, dimethyl sulfoxide-d₆) δ |
| | | 13.18 (s, 1H), 10.55 (s, 1H), 10.18 (s, 1H), 9.33 |
| | | (s, 1H), 8.54 (t, J = 8.3 Hz, 1H), 8.29 (d, J = |
| | | 62.6 Hz, 2H), 7.71 − 7.53 (m, 3H), 7.49 (s, 1H), |
| | | 7.12 (dd, J = 8.6, 1.3 Hz, 1H), 5.64 (h, J = 6.2 |
| | | Hz, 1H), 3.98 (s, 3H), 3.91 (t, J = 6.7 Hz, 2H), |
| | | 3.77 (q, J = 7.3 Hz, 1H), 3.65 (dd, J = 22.0, |
| | | 10.0 Hz, 3H), 3.51 (d, J = 11.1 Hz, 1H), 3.21 − |
| | | 3.06 (m, 2H), 2.76 (t, J = 6.6 Hz, 2H), 2.40 − |
| | | 2.24 (m, 3H), 2.14 − 2.02 (m, 1H), 1.77 − 1.41 |
| | | (m, 5H), 1.37 (d, J = 6.1 Hz, 6H). |
| 87 | 840.3 | ¹H NMR (400 MHz, dimethyl sulfoxide-d₆) δ |
| | | 13.19 (s, 1H), 10.35 (d, J = 156.6 Hz, 2H), 9.33 |
| | | (s, 1H), 8.55 (t, J = 8.4 Hz, 1H), 8.29 (d, J = |
| | | 60.0 Hz, 2H), 7.70 − 7.50 (m, 3H), 7.46 (s, 1H), |
| | | 7.04 (dd, J = 8.5, 1.3 Hz, 1H), 5.64 (h, J = 6.1 |
| | | Hz, 1H), 3.97 (s, 3H), 3.91 (t, J = 6.7 Hz, 2H), |
| | | 3.61 (d, J = 10.9 Hz, 1H), 3.51 (d, J = 11.1 Hz, |
| | | 1H), 2.94 (dd, J = 31.5, 10.8 Hz, 2H), 2.75 (t, |
| | | J = 6.7 Hz, 2H), 2.69 − 2.57 (m, 1H), 2.40 (t, J = |
| | | 11.1 Hz, 1H), 2.16 (dt, J = 30.8, 11.7 Hz, 3H), |
| | | 2.08 − 1.90 (m, 2H), 1.87 − 1.60 (m, 7H), 1.50 |
| | | (d, J = 11.5 Hz, 1H), 1.37 (d, J = 6.1 Hz, 6H). |
| 88 | 853.3 | ¹H NMR (400 MHz, dimethyl sulfoxide-d₆) δ |
| | | 9.25 (d, J = 6.7 Hz, 1H), 8.54 (dt, J = 12.6, 8.5 |
| | | Hz, 1H), 8.32 (s, 2H), 7.65 − 7.53 (m, 2H), 7.44 |
| | | (dd, J = 9.0, 4.0 Hz, 1H), 6.92 (t, J = 8.9 Hz, |
| | | 1H), 6.61 (s, 1H), 5.60 (ddd, J = 12.4, 8.3, 5.2 |
| | | Hz, 1H), 3.91 − 3.82 (m, 5H), 3.70 − 3.45 (m, |
| | | 2H), 3.42 − 3.28 (m, 1H), 3.25 − 3.12 (m, 1H), |
| | | 2.83 − 2.60 (m, 4H), 2.42 (m, 4H), 2.19 (t, J = |
| | | 41.66, 10.59 Hz, 2H), 1.91 (s, 1H), 1.72 (d, J = |
| | | 12.7 Hz, 2H), 1.51 (d, J = 12.6 Hz, 1H), 1.37 (t, |
| | | J = 4.8 Hz, 6H), 1.06 − 0.86 (m, 2H), 0.87 − |
| | | 0.68 (m, 1H), 0.22 (dd, J = 26.0, 4.8 Hz, 1H). |
| 89 | 804.2 | ¹H NMR (400 MHz, dimethyl sulfoxide-d₆) δ |
| | | 13.18 (s, 1H), 10.54 (s, 1H), 10.18 (s, 1H), 9.31 |
| | | (s, 1H), 8.50 (t, J = 8.5 Hz, 1H), 8.28 (s, 2H), |
| | | 7.84 (q, J = 6.5, 4.4 Hz, 4H), 7.54 (t, J = 5.7 Hz, |
| | | 3H), 7.36 (s, 1H), 6.97 (d, J = 8.5 Hz, 1H), 5.61 |
| | | (p, J = 6.2 Hz, 1H), 3.96 (s, 3H), 3.90 (t, J = 6.7 |
| | | Hz, 2H), 3.45 (q, J = 8.9 Hz, 1H), 2.85 (d, J = |
| | | 7.2 Hz, 2H), 2.75 (t, J = 6.7 Hz, 2H), 2.64 − |
| | | 2.54 (m, 1H), 2.46 − 2.36 (m, 2H), 1.90 (q, J = |
| | | 10.0 Hz, 2H), 1.35 (d, J = 6.1 Hz, 6H). |
| 90 | 804.2 | ¹H NMR (400 MHz, dimethyl sulfoxide-d₆) δ |
| | | 13.18 (s, 1H), 10.53 (s, 1H), 10.18 (s, 1H), 9.31 |
| | | (s, 1H), 8.51 (t, J = 8.5 Hz, 1H), 8.28 (d, J = |
| | | 48.6 Hz,2H), 7.97 − 7.74 (m, 4H), 7.61 − 7.47 |
| | | (m, 3H), 7.41 (s, 1H), 7.01 (d, J = 8.5 Hz, 1H), |
| | | 5.62 (p, J = 6.2 Hz, 1H), 3.95 (s, 3H), 3.89 (t, J = |
| | | 6.6 Hz, 2H), 3.80 (q, J = 8.5 Hz, 1H), 3.01 (d, |
| | | J = 7.9 Hz, 2H), 2.74 (t, J = 6.7 Hz, 2H), 2.60 |
| | | (s, 1H), 2.36 − 2.10 (m, 4H), 1.35 (d, J = 6.1 |
| | | Hz, 6H). |
| 91 | 855.2 | ¹H NMR (300 MHz, dimethyl sulfoxide-d₆) δ |
| | | 13.20 (s, 1H), 10.52 (s, 1H), 10.20 (s, 1H), 9.34 |
| | | (d, J = 1.0 Hz, 1H), 8.55 (t, J = 8.2 Hz, 1H), |
| | | 8.30 (d, J = 53.0 Hz, 2H), 7.58 (t, J = 9.9 Hz, |
| | | 2H), 7.45 (d, J = 9.0 Hz, 1H), 6.83 (dd, J = |
| | | 38.5, 7.6 Hz, 2H), 5.65 (p, J = 6.1 Hz, 1H), 4.14 |
| | | (s, 1H), 3.89 (d, J = 5.0 Hz, 5H), 3.57 (ddd, J = |
| | | 33.4, 19.8, 11.2 Hz, 2H), 3.06 (t, J = 11.5 Hz, |
| | | 1H), 2.94 − 2.60 (m, 4H), 2.47 − 2.30 (m, 2H), |
| | | 2.31 − 1.99 (m, 4H), 1.93 − 1.43 (m, 4H), 1.37 |
| | | (d, J = 6.1 Hz, 6H), 1.05 (d, J = 6.2 Hz, 4H). |
| 92 | 854.3 | ¹H NMR (400 MHz, dimethyl sulfoxide-d₆) δ |
| | | 13.19 (s, 1H), 10.55 (s, 1H), 10.18 (s, 1H), 9.34 |
| | | (d, J = 2.8 Hz, 1H), 8.55 (t, J = 8.3 Hz, 1H), |
| | | 8.29 (d, J = 64.0 Hz, 2H), 7.68 − 7.43 (m, 3H), |
| | | 7.35 (d, J = 3.2 Hz, 1H), 7.01 (d, J = 8.6 Hz, |
| | | 1H), 5.64 (pd, J = 6.1, 2.7 Hz, 1H), 3.98 (s, |
| | | 3H), 3.91 (t, J = 6.7 Hz, 2H), 3.58 (ddd, J = |
| | | 42.6, 28.9, 11.1 Hz, 2H), 2.96 (dd, J = 36.3, |
| | | 11.1 Hz, 2H), 2.84 − 2.63 (m, 3H), 2.41 (dd, J = |
| | | 19.1, 9.4 Hz, 1H), 2.31 − 1.93 (m, 7H), 1.86 − |
| | | 1.73 (m, 1H), 1.74 − 1.58 (m, 3H), 1.56 − 1.44 |
| | | (m, 1H), 1.36 (dd, J = 6.1, 2.8 Hz, 6H), 0.95 (q, |
| | | J = 12.6, 11.8 Hz, 1H), 0.75 (d, J = 6.8 Hz, 3H). |
| 93 | 868.3 | ¹H NMR (400 MHz, dimethyl sulfoxide-d₆) δ |
| | | 13.19 (s, 1H), 10.54 (s, 1H), 10.18 (s, 1H), 9.33 |
| | | (d, J = 5.0 Hz, 1H), 8.55 (dd, J = 9.2, 7.4 Hz, |
| | | 1H), 8.29 (d, J = 43.6 Hz, 2H), 7.69 − 7.40 (m, |
| | | 4H), 7.03 (dd, J = 12.8, 8.5 Hz, 1H), 5.73 − |
| | | 5.56 (m, 1H), 3.98 (d, J = 10.3 Hz, 3H), 3.91 (q, |
| | | J = 6.4 Hz, 2H), 3.62 (dd, J = 87.8, 10.9 Hz, |
| | | 1H), 3.41 (d, J = 18.8 Hz, 1H), 2.90 (d, J = 6.4 |
| | | Hz, 1H), 2.75 (td, J = 6.6, 3.4 Hz, 3H), 2.54 (s, |
| | | 1H), 2.39 (dd, J = 23.5, 11.2 Hz, 2H), 2.12 (dt, |
| | | J = 50.9, 10.5 Hz, 1H), 1.90 − 1.42 (m, 9H), |
| | | 1.37 (dd, J = 6.1, 3.5 Hz, 6H), 1.07 (d, J = 15.9 |
| | | Hz, 3H), 0.99 (d, J = 4.5 Hz, 3H). |
| 94 | 840.3 | ¹H NMR (400 MHz, dimethyl sulfoxide-d₆) δ |
| | | 13.19 (s, 1H), 10.54 (s, 1H), 10.17 (s, 1H), 9.33 |
| | | (s, 1H), 8.55 (t, J = 8.3 Hz, 1H), 8.29 (d, J = |
| | | 45.8 Hz, 2H), 7.71 − 7.49 (m, 3H), 7.46 (s, 1H), |
| | | 7.04 (d, J = 8.6 Hz, 1H), 5.64 (p, J = 6.1 Hz, |
| | | 1H), 3.97 (s, 3H), 3.90 (t, J = 6.7 Hz, 2H), 3.60 |
| | | (d, J = 11.1 Hz, 1H), 3.50 (s, 1H), 2.98 (d, J = |
| | | 10.6 Hz, 1H), 2.90 (d, J = 10.7 Hz, 1H), 2.75 (t, |
| | | J = 6.6 Hz, 2H), 2.69 − 2.58 (m, 1H), 2.40 (t, J = |
| | | 11.2 Hz, 1H), 2.25 − 2.08 (m, 3H), 2.01 (ddd, |
| | | J = 27.7, 16.4, 7.5 Hz, 2H), 1.88 − 1.59 (m, |
| | | 7H), 1.50 (d, J = 11.9 Hz, 1H), 1.37 (d, J = 6.1 |
| | | Hz, 6H). |
| 95 | 840.3 | ¹H NMR (400 MHz, dimethyl sulfoxide-d₆) δ |
| | | 13.19 (s, 1H), 10.54 (s, 1H), 10.17 (s, 1H), 9.33 |
| | | (s, 1H), 8.55 (t, J = 8.3 Hz, 1H), 8.29 (s, 2H), |
| | | 7.65 − 7.51 (m, 3H), 7.46 (s, 1H), 7.04 (d, J = |
| | | 8.5 Hz, 1H), 5.64 (p, J = 6.2 Hz, 1H), 3.97 (s, |
| | | 3H), 3.91 (t, J = 6.6 Hz, 2H), 3.61 (d, J = 11.1 |
| | | Hz, 1H), 3.52 (d, J = 9.7 Hz, 1H), 2.98 (d, J = |
| | | 10.8 Hz, 1H), 2.90 (d, J = 11.0 Hz, 1H), 2.75 (t, |
| | | J = 6.7 Hz, 2H), 2.70 − 2.56 (m, 1H), 2.40 (t, |
| | | J = 11.3 Hz, 1H), 2.25 − 2.08 (m, 3H), 2.10 − |
| | | 1.91 (m, 2H), 1.86 − 1.60 (m, 7H), 1.57 − 1.43 |
| | | (m, 1H), 1.37 (d, J = 6.1 Hz, 6H). |
| 96 | 868.3 | ¹H NMR (400 MHz, dimethyl sulfoxide-d₆) δ |
| | | 13.19 (s, 1H), 10.54 (s, 1H), 10.17 (s, 1H), 9.34 |
| | | (s, 1H), 8.55 (t, J = 8.5 Hz, 1H), 8.29 (d, J = |
| | | 64.6 Hz, 2H), 7.65 − 7.57 (m, 2H), 7.53 (d, J = |
| | | 8.5 Hz, 1H), 7.42 (s, 1H), 7.02 (d, J = 8.6 Hz, |
| | | 1H), 5.64 (h, J = 6.2 Hz, 1H), 3.96 (s, 3H), 3.90 |
| | | (t, J = 6.7 Hz, 2H), 3.47 − 3.36 (m, 2H), 2.95 − |
| | | 2.84 (m, 1H), 2.75 (t, J = 6.6 Hz, 3H), 2.61 − |
| | | 2.53 (m, 1H), 2.47 − 2.38 (m, 2H), 2.18 (t, J = |
| | | 10.4 Hz, 1H), 1.92 − 1.74 (m, 3H), 1.74 − 1.61 |
| | | (m, 3H), 1.58 (d, J = 8.2 Hz, 2H), 1.46 (d, J = |
| | | 12.3 Hz, 1H), 1.37 (d, J = 6.1 Hz, 6H), 1.05 (s, |
| | | 3H), 1.00 (s, 3H). |
| 97 | 868.3 | ¹H NMR (400 MHz, dimethyl sulfoxide-d₆) δ |
| | | 13.19 (s, 1H), 10.55 (s, 1H), 10.18 (s, 1H), 9.33 |
| | | (d, J = 1.8 Hz, 1H), 8.55 (t, J = 8.3 Hz, 1H), |
| | | 8.29 (s, 2H), 7.58 (dd, J = 14.8, 9.2 Hz, 3H), |
| | | 7.46 (s, 1H), 7.05 (d, J = 8.5 Hz, 1H), 5.64 (p, |
| | | J = 6.2 Hz, 1H), 3.99 (s, 3H), 3.91 (t, J = 6.7 Hz, |
| | | 2H), 3.73 (d, J = 10.9 Hz, 1H), 3.51 (d, J = 11.1 |
| | | Hz, 1H), 2.91 (s, 1H), 2.82 − 2.70 (m, 3H),, |
| | | 2.55 (d, J = 14.0 Hz, 1H), 2.38 (s, 2H), 2.06 (t, |
| | | J = 11.0 Hz, 1H), 1.81 (t, J = 12.5 Hz, 2H), 1.76 − |
| | | 1.59 (m, 5H), 1.59 − 1.42 (m, 2H), 1.36 (d, J = |
| | | 6.1 Hz, 6H), 1.09 (s, 3H), 0.99 (s, 3H). |
| 98 | 848.2 | ¹H NMR (400 MHz, dimethyl sulfoxide-d₆) δ |
| | | 13.16 (s, 1H), 10.37 (d, J = 96.2 Hz, 2H), 9.29 |
| | | (s, 1H), 8.50 (d, J = 8.2 Hz, 1H), 8.27 (s, 2H), |
| | | 7.95 (s, 1H), 7.90 − 7.74 (m, 3H), 7.66 (d, J = |
| | | 7.6 Hz, 1H), 7.60 (t, J = 7.7 Hz, 1H), 7.39 (d, J = |
| | | 9.1 Hz, 1H), 6.84 (d, J = 9.0 Hz, 1H), 6.77 (s, |
| | | 1H), 5.69 − 5.48 (m, 1H), 3.86 (d, J = 6.1 Hz, |
| | | 5H), 3.71 − 3.59 (m, 1H), 3.19 (s, 4H), 2.71 (t, |
| | | J = 6.6 Hz, 2H), 2.63 − 2.52 (m, 2H), 2.48 − 2.38 |
| | | (m, 2H), 1.35 (d, J = 6.2 Hz, 9H). |
| 99 | 848.2 | ¹H NMR (400 MHz, dimethyl sulfoxide-d₆) δ |
| | | 13.19 (s, 1H), 10.50 (s, 1H), 10.24 (s, 1H), 9.29 |
| | | (s, 1H), 8.51 (t, J = 8.2 Hz, 1H), 8.27 (s, 2H), |
| | | 7.95 (s, 1H), 7.90 − 7.78 (m, 3H), 7.67 (d, J = |
| | | 7.8 Hz, 1H), 7.60 (t, J = 7.7 Hz, 1H), 7.39 (d, J = |
| | | 9.0 Hz, 1H), 6.85 (d, J = 9.1 Hz, 1H), 6.78 (d, |
| | | J = 1.8 Hz, 1H), 5.61 (p, J = 6.2 Hz, 1H), 3.85 |
| | | (s, 5H), 3.66 (t, J = 6.7 Hz, 1H), 3.19 (s, 4H), |
| | | 2.71 (t, J = 6.6 Hz, 2H), 2.61 − 2.51 (m, 2H), |
| | | 2.43 (ddd, J = 10.7, 5.6, 3.5 Hz, 2H), 1.35 (d, |
| | | J = 6.2 Hz, 9H). |
| 100 | 847.3 | ¹H NMR (400 MHz, dimethyl sulfoxide-d₆) δ |
| | | 13.16 (s, 1H), 10.54 (s, 1H), 10.24 (d, J = 2.0 |
| | | Hz, 1H), 9.26 (s, 1H), 8.53 (t, J = 8.5 Hz, 1H), |
| | | 8.27 (s, 2H), 8.00 (s, 1H), 7.94 − 7.82 (m, 3H), |
| | | 7.67 (d, J = 26.0 Hz, 2H), 7.51 (d, J = 8.4 Hz, |
| | | 1H), 7.40 (s, 1H), 6.98 (d, J = 8.6 Hz, 1H), 5.60 |
| | | (h, J = 6.3 Hz, 1H), 3.95 (s, 3H), 3.88 (t, J = 6.7 |
| | | Hz, 2H), 3.83 − 3.56 (m, 1H), 3.24 − 2.78 (m, |
| | | 2H), 2.73 (t, J = 6.7 Hz, 2H), 2.64 − 2.53 (m, |
| | | 1H), 2.29 − 1.94 (m, 2H), 1.78 (d, J = 18.1 Hz, |
| | | 4H), 1.53 − 1.27 (m, 9H). |
| 101 | 847.3 | ¹H NMR (400 MHz, dimethyl sulfoxide-d₆) δ |
| | | 13.44 − 12.85 (s, 1H), 10.54 (s, 1H), 10.23 (d, J = |
| | | 2.1 Hz, 1H), 9.26 (s, 1H), 8.53 (t, J = 8.5 Hz, |
| | | 1H), 8.27 (s, 2H), 7.99 (s, 1H), 7.94 − 7.81 (m, |
| | | 3H), 7.77 − 7.59 (m, 2H), 7.51 (d, J = 8.5 Hz, |
| | | 1H), 7.40 (s, 1H), 6.98 (d, J = 8.5 Hz, 1H), 5.60 |
| | | (p, J = 6.1 Hz, 1H), 3.95 (s, 3H), 3.88 (t, J = 6.7 |
| | | Hz, 2H), 3.83 − 3.60 (m, 1H), 3.20 − 2.81 (m, |
| | | 2H), 2.73 (t, J = 6.7 Hz, 2H), 2.59 (s, 1H), 2.21 − |
| | | 1.94 (m, 2H), 1.91 − 1.66 (m, 4H), 1.40 (td, J = |
| | | 10.9, 8.4, 4.8 Hz, 3H), 1.33 (d, J = 6.1 Hz, |
| | | 6H). |
| 102 | 868.3 | ¹H NMR (400 MHz, dimethyl sulfoxide-d₆) δ |
| | | 13.17 (s, 1H), 10.54 (s, 1H), 10.19 (s, 1H), 9.34 |
| | | (d, J = 1.2 Hz, 1H), 8.55 (t, J = 8.3 Hz, 1H), |
| | | 8.31 (s, 2H), 7.80 − 7.48 (m, 3H), 7.42 (s, 1H), |
| | | 7.02 (d, J = 8.5 Hz, 1H), 5.65 (p, J = 5.8 Hz, |
| | | 1H), 4.08 − 3.82 (m, 5H), 3.39 (s, 2H), 2.91 (d, |
| | | J = 13.2 Hz, 1H), 2.75 (t, J = 6.7 Hz, 3H), 2.55 |
| | | (s, 1H), 2.42 (d, J = 9.3 Hz, 2H), 2.19 (t, J = |
| | | 10.4 Hz, 1H), 1.93 − 1.62 (m, 6H), 1.59 (d, J = |
| | | 7.9 Hz, 2H), 1.47 (d, J = 20.3 Hz, 1H), 1.42 − |
| | | 1.33 (m, 6H), 1.03 (d, J = 19.4 Hz, 6H). |
| 103 | 868.3 | ¹H NMR (400 MHz, dimethyl sulfoxide-d₆) δ |
| | | 13.19 (s, 1H), 10.54 (s, 1H), 10.18 (s, 1H), 9.34 |
| | | (s, 1H), 8.55 (t, J = 8.4 Hz, 1H), 8.30 (d, J = |
| | | 61.4 Hz, 2H), 7.74 − 7.46 (m, 3H), 7.42 (s, 1H), |
| | | 7.02 (d, J = 8.5 Hz, 1H), 5.65 (p, J = 6.2 Hz, |
| | | 1H), 3.96 (s, 3H), 3.90 (t, J = 6.6 Hz, 2H), 3.39 |
| | | (s, 2H), 2.89 (s, 1H), 2.75 (t, J = 6.6 Hz, 3H), |
| | | 2.55 (d, J = 6.9 Hz, 1H), 2.45 (s, 2H), 2.18 (t, J = |
| | | 10.5 Hz, 1H), 1.90 − 1.62 (m, 6H), 1.58 (d, J = |
| | | 8.0 Hz, 2H), 1.46 (d, J = 11.5 Hz, 1H), 1.37 |
| | | (d, J = 6.1 Hz, 6H), 1.02 (d, J = 19.4 Hz, 6H). |
| 104 | 868.3 | ¹H NMR (400 MHz, dimethyl sulfoxide-d₆) δ |
| | | 13.20 (s, 1H), 10.55 (s, 1H), 10.18 (s, 1H), 9.32 |
| | | (d, J = 1.8 Hz, 1H), 8.55 (t, J = 8.3 Hz, 1H), |
| | | 8.30 (d, J = 39.3 Hz, 2H), 7.77 − 7.50 (m, 3H), |
| | | 7.45 (s, 1H), 7.05 (d, J = 8.5 Hz, 1H), 5.72 − |
| | | 5.50 (m, 1H), 3.99 (d, J = 1.8 Hz, 3H), 3.91 (t, J = |
| | | 6.5 Hz, 2H), 3.72 (d, J = 10.8 Hz, 1H), 3.50 |
| | | (d, J = 7.7 Hz, 1H), 2.89 (d, J = 12.1 Hz, 1H), |
| | | 2.76 (t, J = 6.2 Hz, 3H), 2.55 (d, J = 13.8 Hz, |
| | | 1H), 2.36 (d, J = 11.8 Hz, 2H), 2.05 (t, J = 10.4 |
| | | Hz, 1H), 1.87 − 1.74 (m, 2H), 1.73 − 1.58 (m, |
| | | 5H), 1.58 − 1.40 (m, 2H), 1.36 (d, J = 6.1 Hz, |
| | | 6H), 1.09 (s, 3H), 0.99 (s, 3H). |
| 105 | 868.3 | ¹H NMR (400 MHz, dimethyl sulfoxide-d₆) δ |
| | | 13.20 (s, 1H), 10.55 (s, 1H), 10.17 (s, 1H), 9.33 |
| | | (s, 1H), 8.55 (t, J = 8.3 Hz, 1H), 8.29 (s, 2H), |
| | | 7.69 − 7.51 (m, 3H), 7.46 (s, 1H), 7.05 (d, J = |
| | | 8.6 Hz, 1H), 5.64 (p, J = 6.2 Hz, 1H), 4.16 − |
| | | 3.95 (m, 3H), 3.96 − 3.85 (m, 2H), 3.73 (d, J = |
| | | 10.7 Hz, 1H), 3.50 (d, J = 5.5 Hz, 1H), 2.92 (d, |
| | | J = 11.1 Hz, 1H), 2.76 (t, J = 6.7 Hz, 3H), 2.64 − |
| | | 2.56 (m, 1H), 2.38 (s, 2H), 2.06 (dd, J = 12.2, |
| | | 7.4 Hz, 1H), 1.83 (d, J = 9.5 Hz, 2H), 1.67 (t, J = |
| | | 14.7 Hz, 5H), 1.59 − 1.40 (m, 2H), 1.36 (d, J = |
| | | 6.1 Hz, 6H), 1.31 − 1.16 (m, 2H), 1.09 (s, |
| | | 3H), 0.99 (s, 3H). |
| 106 | 876.3 | ¹H NMR (400 MHz, dimethyl sulfoxide-d₆) δ |
| | | 13.19 (s, 1H), 10.50 (s, 1H), 10.23 (s, 1H), 9.31 |
| | | (d, J = 1.1 Hz, 1H), 8.53 (t, J = 8.5 Hz, 1H), |
| | | 8.28 (d, J = 68.1 Hz, 2H), 7.95 (s, 1H), 7.91 − |
| | | 7.77 (m, 3H), 7.70 − 7.54 (m, 2H), 7.43 (d, J = |
| | | 9.0 Hz, 1H), 6.95 − 6.73 (m, 2H), 5.62 (p, J = |
| | | 6.1 Hz, 1H), 4.16 (d, J = 14.2 Hz, 1H), 3.88 (d, |
| | | J = 1.9 Hz, 5H), 3.60 − 3.36 (m, 3H), 2.88 − |
| | | 2.67 (m, 5H), 2.34 (dt, J = 22.7, 12.0 Hz, 3H), |
| | | 1.36 (d, J = 6.1 Hz, 6H), 1.02 (d, J = 6.4 Hz, |
| | | 3H), 0.92 (d, J = 6.4 Hz, 3H). |
| 107 | 876.3 | ¹H NMR (400 MHz, dimethyl sulfoxide-d₆) δ |
| | | 13.19 (s, 1H), 10.50 (s, 1H), 10.23 (s, 1H), 9.31 |
| | | (s, 1H), 8.53 (t, J = 8.5 Hz, 1H), 8.37 (s, 1H), |
| | | 8.19 (s, 1H), 7.95 (t, J = 1.9 Hz, 1H), 7.90 − |
| | | 7.77 (m, 3H), 7.70 − 7.57 (m, 2H), 7.43 (d, J = |
| | | 9.0 Hz, 1H), 6.91 − 6.79 (m, 2H), 5.62 (p, J = |
| | | 6.2 Hz, 1H), 4.16 (d, J = 14.3 Hz, 1H), 3.88 (d, |
| | | J = 1.7 Hz, 5H), 3.56 − 3.37 (m, 3H), 2.87 − |
| | | 2.75 (m, 3H), 2.73 (t, J = 6.7 Hz, 2H), 2.42 − |
| | | 2.22 (m, 3H), 1.36 (d, J = 6.1 Hz, 6H), 1.02 (d, |
| | | J = 6.4 Hz, 3H), 0.92 (d, J = 6.4 Hz, 3H). |
| 108 | 876.3 | ¹H NMR (400 MHz, dimethyl sulfoxide-d₆) δ |
| | | 13.13 (s, 1H), 10.41 (s, 1H), 10.16 (s, 1H), 9.23 |
| | | (s, 1H), 8.48 (t, J = 8.5 Hz, 1H), 8.21 (d, J = |
| | | 69.0 Hz, 2H), 8.03 − 7.65 (m, 4H), 7.59 − 7.40 |
| | | (m, 2H), 7.28 (d, J = 9.0 Hz, 1H), 6.74 (d, J = |
| | | 9.2 Hz, 1H), 6.61 (s, 1H), 5.55 (p, J = 6.1 Hz, |
| | | 1H), 3.80 (t, J = 6.7 Hz, 2H), 3.76 (s, 3H), 3.62 |
| | | (d, J = 13.6 Hz, 1H), 3.56 (s, 1H), 3.53 (s, 1H), |
| | | 3.51 (s, 1H), 3.26 (d, J = 48.4 Hz, 1H), 2.74 (d, |
| | | J = 10.5 Hz, 1H), 2.65 (t, J = 6.8 Hz, 3H), 2.42 − |
| | | 2.32 (m, 1H), 2.15 (s, 1H), 2.00 (d, J = 10.9 |
| | | Hz, 1H), 1.29 (d, J = 6.1 Hz, 6H), 0.59 (dd, J = |
| | | 6.7, 4.5 Hz, 6H). |
| 109 | 876.3 | ¹H NMR (400 MHz, dimethyl sulfoxide-d₆) δ |
| | | 13.12 (s, 1H), 10.41 (s, 1H), 10.17 (s, 1H), 9.24 |
| | | (s, 1H), 8.47 (t, J = 8.5 Hz, 1H), 8.30 (s, 1H), |
| | | 8.13 (s, 1H), 7.90 − 7.70 (m, 4H), 7.63 − 7.50 |
| | | (m, 2H), 7.28 (d, J = 9.0 Hz, 1H), 6.74 (dd, J = |
| | | 9.3, 2.0 Hz, 1H), 6.61 (d, J = 2.0 Hz, 1H), 5.55 |
| | | (p, J = 6.1 Hz, 1H), 3.80 (t, J = 6.7 Hz, 2H), |
| | | 3.78 − 3.74 (m, 3H), 3.62 (d, J = 13.6 Hz, 1H), |
| | | 3.60 − 3.44 (m, 3H), 3.20 (t, J = 11.8 Hz, 1H), |
| | | 2.74 (d, J = 10.5 Hz, 1H), 2.66 (q, J = 6.7, 6.0 |
| | | Hz, 3H), 2.37 (d, J = 7.3 Hz, 1H), 2.19 − 2.09 |
| | | (m, 1H), 2.04 − 1.91 (m, 1H), 1.29 (d, J = 6.1 |
| | | Hz, 6H), 0.60 (dd, J = 6.8, 3.4 Hz, 6H). |

### Biological Assays

### HiBit Assay Protocol for CDK2

### 1. Materials PotoN1046,T 3

### 1.1 Reagents

| Reagents | Vendor | Cat# |
|---|---|---|
| HEK293-CDK2-Hibit cell | Pharmaron | \ |
| DMEM | Hyclone | SH30022 01 |
| FBS | Gibco | 10099141C |
| Nano-Glo ® HiBiT Lytic | Promega | N3040 |
| Detection System | | |
| 0.25% Trypsin/EDTA | Invitrogen | #25300 |
| PBS | Solarbio | P1020-500 |
| Penicillin-Streptomycin Liquid | Solarbio | P1400 |

### 1.2 Instruments

| Instrument | Vendor | Cat# |
|---|---|---|
| Centrifuge | Eppendorf | 5810R |
| CO₂ Incubator | Thermo | Model: 371 |
| Vortex | IKA | MS3 digital |
| Microplate shaker | Yoning | WZ-4 |
| Echo Liquid Handler | Labcyte | 550 |
| TECAN | TECAN | Freedom EVO200 |
| 384 well plate | Corning | 3570 |
| 384 pp-plate | Labcyte | 001-14555 |
| 50 mL centrifuge tube | BD-Falcon | 352098 |
| 15 mL centrifuge tube | BD-Falcon | 352097 |
| EnVision | PerkinElmer | 2105-0020 |

2. Assay procedure

### Day 1.

1. The culture medium for HEK293-CDK2-Hibit cells was prepared using DMEM with 10% FBS.
2. Cells were cultivated in T-75 flasks in a cell culture incubator set at 37° C., 5% CO2, 95% relative humidity. Cells were allowed to reach 80-90% confluence before detaching and splitting.
3. Cultivated cells were rinsed in T-75 flasks with 5 mL PBS. Aspirated off. Added 1.5 mL trypsin, and incubated the cells at 37° C. for approximately 5 minutes or until the cells detached and floated.
4. Trypsin was inactivated by adding excess serum containing medium.
5. Cells were harvested from flask into cell culture medium and then the cell number was counted. HEK293-CDK2-Hibit cells were seeded into 384-well plate (cell density: 1.6×105 cells/well/50 uL medium) in DMEM medium according to the plate map below and were incubated overnight at 37° C. and 5% CO2.
Note: Cell Culture Medium: 90% DMEM+10% FBS+1% Penicillin-Streptomycin Liquid

### Plate Map

Plate map

| Top(nM) | | 1 | 2 | 3 | 4 | 5 | 6 | 7 | 8 | 9 | 10 | 11 | 12 |
|---|---|---|---|---|---|---|---|---|---|---|---|---|---|
| | A | | | | | | | | | | | | |
| [symbol] 0000 | B | NC | [symbol] 0000 | 3333 | 1111 | 370 | [symbol] 23 | 4 [symbol] .2 | [symbol] 3.7 | 4. [symbol] | [symbol] .5 | 0.5 [symbol] | PC |
| [symbol] 0000 | C | NC | [symbol] 0000 | 3333 | 1111 | 370 | [symbol] 23 | 4 [symbol] .2 | [symbol] 3.7 | 4. [symbol] | [symbol] .5 | 0.5 [symbol] | PC |
| [symbol] 0000 | D | NC | [symbol] 0000 | 3333 | 1111 | 370 | [symbol] 23 | 4 [symbol] .2 | [symbol] 3.7 | 4. [symbol] | [symbol] .5 | 0.5 [symbol] | PC |
| [symbol] 0000 | E | NC | [symbol] 0000 | 3333 | 1111 | 370 | [symbol] 23 | 4 [symbol] .2 | [symbol] 3.7 | 4. [symbol] | [symbol] .5 | 0.5 [symbol] | PC |
| [symbol] 0000 | F | NC | [symbol] 0000 | 3333 | 1111 | 370 | [symbol] 23 | 4 [symbol] .2 | [symbol] 3.7 | 4. [symbol] | [symbol] .5 | 0.5 [symbol] | PC |
| [symbol] 0000 | G | NC | [symbol] 0000 | 3333 | 1111 | 370 | [symbol] 23 | 4 [symbol] .2 | [symbol] 3.7 | 4. [symbol] | [symbol] .5 | 0.5 [symbol] | PC |
| [symbol] 0000 | H | NC | [symbol] 0000 | 3333 | 1111 | 370 | [symbol] 23 | 4 [symbol] .2 | [symbol] 3.7 | 4. [symbol] | [symbol] .5 | 0.5 [symbol] | PC |
| [symbol] 0000 | I | NC | [symbol] 0000 | 3333 | 1111 | 370 | [symbol] 23 | 4 [symbol] .2 | [symbol] 3.7 | 4. [symbol] | [symbol] .5 | 0.5 [symbol] | PC |
| [symbol] 0000 | J | NC | [symbol] 0000 | 3333 | 1111 | 370 | [symbol] 23 | 4 [symbol] .2 | [symbol] 3.7 | 4. [symbol] | [symbol] .5 | 0.5 [symbol] | PC |
| [symbol] 0000 | K | NC | [symbol] 0000 | 3333 | 1111 | 370 | [symbol] 23 | 4 [symbol] .2 | [symbol] 3.7 | 4. [symbol] | [symbol] .5 | 0.5 [symbol] | PC |
| [symbol] 0000 | L | NC | [symbol] 0000 | 3333 | 1111 | 370 | [symbol] 23 | 4 [symbol] .2 | [symbol] 3.7 | 4. [symbol] | [symbol] .5 | 0.5 [symbol] | PC |
| [symbol] 0000 | M | NC | [symbol] 0000 | 3333 | 1111 | 370 | [symbol] 23 | 4 [symbol] .2 | [symbol] 3.7 | 4. [symbol] | [symbol] .5 | 0.5 [symbol] | PC |
| [symbol] 0000 | N | NC | [symbol] 0000 | 3333 | 1111 | 370 | [symbol] 23 | 4 [symbol] .2 | [symbol] 3.7 | 4. [symbol] | [symbol] .5 | 0.5 [symbol] | PC |
| [symbol] 0000 | O | NC | [symbol] 0000 | 3333 | 1111 | 370 | [symbol] 23 | 4 [symbol] .2 | [symbol] 3.7 | 4. [symbol] | [symbol] .5 | 0.5 [symbol] | PC |
| | P | | | | | | | | | | | | |
| Top(nM) | | 13 | 14 | 15 | 16 | 17 | 18 | 19 | 20 | 21 | 22 | 23 | 24 |
| | A | | | | | | | | | | | | |
| [symbol] 0000 | B | PC | [symbol] 0000 | 3333 | 1111 | 370 | [symbol] 23 | 4 [symbol] .2 | [symbol] 3.7 | 4. [symbol] | [symbol] .5 | 0.5 [symbol] | NC |
| [symbol] 0000 | C | PC | [symbol] 0000 | 3333 | 1111 | 370 | [symbol] 23 | 4 [symbol] .2 | [symbol] 3.7 | 4. [symbol] | [symbol] .5 | 0.5 [symbol] | NC |
| [symbol] 0000 | D | PC | [symbol] 0000 | 3333 | 1111 | 370 | [symbol] 23 | 4 [symbol] .2 | [symbol] 3.7 | 4. [symbol] | [symbol] .5 | 0.5 [symbol] | NC |
| [symbol] 0000 | E | PC | [symbol] 0000 | 3333 | 1111 | 370 | [symbol] 23 | 4 [symbol] .2 | [symbol] 3.7 | 4. [symbol] | [symbol] .5 | 0.5 [symbol] | NC |
| [symbol] 0000 | F | PC | [symbol] 0000 | 3333 | 1111 | 370 | [symbol] 23 | 4 [symbol] .2 | [symbol] 3.7 | 4. [symbol] | [symbol] .5 | 0.5 [symbol] | NC |
| [symbol] 0000 | G | PC | [symbol] 0000 | 3333 | 1111 | 370 | [symbol] 23 | 4 [symbol] .2 | [symbol] 3.7 | 4. [symbol] | [symbol] .5 | 0.5 [symbol] | NC |
| [symbol] 0000 | H | PC | [symbol] 0000 | 3333 | 1111 | 370 | [symbol] 23 | 4 [symbol] .2 | [symbol] 3.7 | 4. [symbol] | [symbol] .5 | 0.5 [symbol] | NC |
| [symbol] 0000 | I | PC | [symbol] 0000 | 3333 | 1111 | 370 | [symbol] 23 | 4 [symbol] .2 | [symbol] 3.7 | 4. [symbol] | [symbol] .5 | 0.5 [symbol] | NC |
| [symbol] 0000 | J | PC | [symbol] 0000 | 3333 | 1111 | 370 | [symbol] 23 | 4 [symbol] .2 | [symbol] 3.7 | 4. [symbol] | [symbol] .5 | 0.5 [symbol] | NC |
| [symbol] 0000 | K | PC | [symbol] 0000 | 3333 | 1111 | 370 | [symbol] 23 | 4 [symbol] .2 | [symbol] 3.7 | 4. [symbol] | [symbol] .5 | 0.5 [symbol] | NC |
| [symbol] 0000 | L | PC | [symbol] 0000 | 3333 | 1111 | 370 | [symbol] 23 | 4 [symbol] .2 | [symbol] 3.7 | 4. [symbol] | [symbol] .5 | 0.5 [symbol] | NC |
| [symbol] 0000 | M | PC | [symbol] 0000 | 3333 | 1111 | 370 | [symbol] 23 | 4 [symbol] .2 | [symbol] 3.7 | 4. [symbol] | [symbol] .5 | 0.5 [symbol] | NC |
| [symbol] 0000 | N | PC | [symbol] 0000 | 3333 | 1111 | 370 | [symbol] 23 | 4 [symbol] .2 | [symbol] 3.7 | 4. [symbol] | [symbol] .5 | 0.5 [symbol] | NC |
| [symbol] 0000 | O | PC | [symbol] 0000 | 3333 | 1111 | 370 | [symbol] 23 | 4 [symbol] .2 | [symbol] 3.7 | 4. [symbol] | [symbol] .5 | 0.5 [symbol] | NC |
| | P | | | | | | | | | | | | |

[symbol]

indicates data missing or illegible when filed

### Day 2,

1 Test compounds were dissolved at 10 mM DMSO stock solution 45 uL of stock solution was transferred to 384 pp-plate. A 3-fold, 10-point dilution was performed via transferring 15 uL compound into 30 uL DMSO by using TECAN liquid handler.
2. The plates were spinned at room temperature at 1,000 RPM for 1 minute.
3. 50 nL of diluted compound was transferred from compound source plate into the cell plate by Echo.
4. After compound treatment for 6 hours, the plate was removed from incubators and equilibrated at room temperature for 15 minutes. The HiBit reagent was prepared, consisting of the following ratios: 10 mL of Nano-Glo®-HiBiT Lytic Buffer, 200 μL of Nano-Glo®-HiBiT Lytic Substrate and 100 μL of LgBiT Protein, mixed thoroughly. 20 μL of HiBit reagent was added into each well and the plates were spinned at room temperature at 1,000 RPM for 1 minute to be detected. Then the plates were shaken at 600 RPM at room temperature for 20 minutes, and read by EnVision.
5. The inhibition activity was calculated following the formula below:

$$\% Degrader = 100 \times ( LumHC - LumSample ) / ( ( LumHC - LumLC )$$

Note: Where HC was obtained from cells treated with 0.1% DMSO only; LC was obtained from culture medium only.
6. ABS DC₅₀ was calculated by fitting the Curve using Xlfit (v5.3.1.3), equation 201:

$$Y = Bottom + ( Top - Bottom ) / ( 1 + 1 {0}^{∧} ( ( Log IC 50 - X ) * HillSlope ) )$$

The data from this assay is presented in Table 1, columns 3 and 4.

### HiBit Assay Protocol for CCNE1

### 1. Materials

| Reagents | Vendor | Cat# |
|---|---|---|
| HEK293-CCNE1-Hibit cell | Pharmaron | \ |
| EMEM | ATCC | 30-2003 |
| FBS | Gibco | 10099141C |
| Nano-Glo ® HiBiT Lytic | Promega | N3040 |
| Detection System | | |
| 0.25% Trypsin/EDTA | Invitrogen | #25300 |
| PBS | Solarbio | P1020-500 |
| Penicillin-Streptomycin Liquid | Solarbio | P1400 |

| Instrument | Vendor | Cat# |
|---|---|---|
| Centrifuge | Eppendorf | 5810R |
| CO₂ Incubator | Thermo | Model: 371 |
| Vortex | IKA | MS3 digital |
| Microplate shaker | Yoning | WZ-4 |
| Echo Liquid Handler | Labcyte | 550 |
| TECAN | TECAN | Freedom EVO200 |
| 384 well plate | Corning | 3570 |
| 384 pp-plate | Labcyte | 001-14555 |
| 50 mL centrifuge tube | BD-Falcon | 352098 |
| 15 mL centrifuge tube | BD-Falcon | 352097 |
| EnVision | PerkinElmer | 2105-0020 |

### 2. Assay Procedure

### Day 1.

1. The culture medium for HEK293-CCNE1-Hibit cell was prepared using EMEM with 10% FBS.
2. Cells were cultivated in T-75 flasks in a cell culture incubator set at 37° C., 5% CO2, 95% relative humidity. Cells were allowed to reach 80-90% confluence before detaching and splitting.
3. Cultivated cells were rinsed in T-75 flasks with 5 mL PBS. Aspirated off. Added was 1.5 mL trypsin, and incubated the cells at 37° C. for approximately 5 minutes or until the cells detached and floated.
4. Trypsin was inactivated by adding excess serum containing medium.
5. Cells were harvested from flask into cell culture medium and then the cell number was counted. HEK293-CCNE1-Hibit cell were seeded into 384-well plate (cell density: 1.6×10⁵ cells/well/50 uL medium) in EMEM medium according to the plate map below and incubated overnight at 37° C. and 5% CO₂.

Note: Cell Culture Medium: 90% EMEM+10% FBS+1% Penicillin-Streptomycin Liquid

### Plate Map

Plate map

| Top(nM) | | 1 | 2 | 3 | 4 | 5 | 6 | 7 | 8 | 9 | 10 | 11 | 12 |
|---|---|---|---|---|---|---|---|---|---|---|---|---|---|
| | A | | | | | | | | | | | | |
| [symbol] 0000 | B | NC | [symbol] 0000 | 3333 | 1111 | 370 | [symbol] 23 | 4 [symbol] .2 | [symbol] 3.7 | 4. [symbol] | [symbol] .5 | 0.5 [symbol] | PC |
| [symbol] 0000 | C | NC | [symbol] 0000 | 3333 | 1111 | 370 | [symbol] 23 | 4 [symbol] .2 | [symbol] 3.7 | 4. [symbol] | [symbol] .5 | 0.5 [symbol] | PC |
| [symbol] 0000 | D | NC | [symbol] 0000 | 3333 | 1111 | 370 | [symbol] 23 | 4 [symbol] .2 | [symbol] 3.7 | 4. [symbol] | [symbol] .5 | 0.5 [symbol] | PC |
| [symbol] 0000 | E | NC | [symbol] 0000 | 3333 | 1111 | 370 | [symbol] 23 | 4 [symbol] .2 | [symbol] 3.7 | 4. [symbol] | [symbol] .5 | 0.5 [symbol] | PC |
| [symbol] 0000 | F | NC | [symbol] 0000 | 3333 | 1111 | 370 | [symbol] 23 | 4 [symbol] .2 | [symbol] 3.7 | 4. [symbol] | [symbol] .5 | 0.5 [symbol] | PC |
| [symbol] 0000 | G | NC | [symbol] 0000 | 3333 | 1111 | 370 | [symbol] 23 | 4 [symbol] .2 | [symbol] 3.7 | 4. [symbol] | [symbol] .5 | 0.5 [symbol] | PC |
| [symbol] 0000 | H | NC | [symbol] 0000 | 3333 | 1111 | 370 | [symbol] 23 | 4 [symbol] .2 | [symbol] 3.7 | 4. [symbol] | [symbol] .5 | 0.5 [symbol] | PC |
| [symbol] 0000 | I | NC | [symbol] 0000 | 3333 | 1111 | 370 | [symbol] 23 | 4 [symbol] .2 | [symbol] 3.7 | 4. [symbol] | [symbol] .5 | 0.5 [symbol] | PC |
| [symbol] 0000 | J | NC | [symbol] 0000 | 3333 | 1111 | 370 | [symbol] 23 | 4 [symbol] .2 | [symbol] 3.7 | 4. [symbol] | [symbol] .5 | 0.5 [symbol] | PC |
| [symbol] 0000 | K | NC | [symbol] 0000 | 3333 | 1111 | 370 | [symbol] 23 | 4 [symbol] .2 | [symbol] 3.7 | 4. [symbol] | [symbol] .5 | 0.5 [symbol] | PC |
| [symbol] 0000 | L | NC | [symbol] 0000 | 3333 | 1111 | 370 | [symbol] 23 | 4 [symbol] .2 | [symbol] 3.7 | 4. [symbol] | [symbol] .5 | 0.5 [symbol] | PC |
| [symbol] 0000 | M | NC | [symbol] 0000 | 3333 | 1111 | 370 | [symbol] 23 | 4 [symbol] .2 | [symbol] 3.7 | 4. [symbol] | [symbol] .5 | 0.5 [symbol] | PC |
| [symbol] 0000 | N | NC | [symbol] 0000 | 3333 | 1111 | 370 | [symbol] 23 | 4 [symbol] .2 | [symbol] 3.7 | 4. [symbol] | [symbol] .5 | 0.5 [symbol] | PC |
| [symbol] 0000 | O | NC | [symbol] 0000 | 3333 | 1111 | 370 | [symbol] 23 | 4 [symbol] .2 | [symbol] 3.7 | 4. [symbol] | [symbol] .5 | 0.5 [symbol] | PC |
| | P | | | | | | | | | | | | |
| Top(nM) | | 13 | 14 | 15 | 16 | 17 | 18 | 19 | 20 | 21 | 22 | 23 | 24 |
| | A | | | | | | | | | | | | |
| [symbol] 0000 | B | PC | [symbol] 0000 | 3333 | 1111 | 370 | [symbol] 23 | 4 [symbol] .2 | [symbol] 3.7 | 4. [symbol] | [symbol] .5 | 0.5 [symbol] | NC |
| [symbol] 0000 | C | PC | [symbol] 0000 | 3333 | 1111 | 370 | [symbol] 23 | 4 [symbol] .2 | [symbol] 3.7 | 4. [symbol] | [symbol] .5 | 0.5 [symbol] | NC |
| [symbol] 0000 | D | PC | [symbol] 0000 | 3333 | 1111 | 370 | [symbol] 23 | 4 [symbol] .2 | [symbol] 3.7 | 4. [symbol] | [symbol] .5 | 0.5 [symbol] | NC |
| [symbol] 0000 | E | PC | [symbol] 0000 | 3333 | 1111 | 370 | [symbol] 23 | 4 [symbol] .2 | [symbol] 3.7 | 4. [symbol] | [symbol] .5 | 0.5 [symbol] | NC |
| [symbol] 0000 | F | PC | [symbol] 0000 | 3333 | 1111 | 370 | [symbol] 23 | 4 [symbol] .2 | [symbol] 3.7 | 4. [symbol] | [symbol] .5 | 0.5 [symbol] | NC |
| [symbol] 0000 | G | PC | [symbol] 0000 | 3333 | 1111 | 370 | [symbol] 23 | 4 [symbol] .2 | [symbol] 3.7 | 4. [symbol] | [symbol] .5 | 0.5 [symbol] | NC |
| [symbol] 0000 | H | PC | [symbol] 0000 | 3333 | 1111 | 370 | [symbol] 23 | 4 [symbol] .2 | [symbol] 3.7 | 4. [symbol] | [symbol] .5 | 0.5 [symbol] | NC |
| [symbol] 0000 | I | PC | [symbol] 0000 | 3333 | 1111 | 370 | [symbol] 23 | 4 [symbol] .2 | [symbol] 3.7 | 4. [symbol] | [symbol] .5 | 0.5 [symbol] | NC |
| [symbol] 0000 | J | PC | [symbol] 0000 | 3333 | 1111 | 370 | [symbol] 23 | 4 [symbol] .2 | [symbol] 3.7 | 4. [symbol] | [symbol] .5 | 0.5 [symbol] | NC |
| [symbol] 0000 | K | PC | [symbol] 0000 | 3333 | 1111 | 370 | [symbol] 23 | 4 [symbol] .2 | [symbol] 3.7 | 4. [symbol] | [symbol] .5 | 0.5 [symbol] | NC |
| [symbol] 0000 | L | PC | [symbol] 0000 | 3333 | 1111 | 370 | [symbol] 23 | 4 [symbol] .2 | [symbol] 3.7 | 4. [symbol] | [symbol] .5 | 0.5 [symbol] | NC |
| [symbol] 0000 | M | PC | [symbol] 0000 | 3333 | 1111 | 370 | [symbol] 23 | 4 [symbol] .2 | [symbol] 3.7 | 4. [symbol] | [symbol] .5 | 0.5 [symbol] | NC |
| [symbol] 0000 | N | PC | [symbol] 0000 | 3333 | 1111 | 370 | [symbol] 23 | 4 [symbol] .2 | [symbol] 3.7 | 4. [symbol] | [symbol] .5 | 0.5 [symbol] | NC |
| [symbol] 0000 | O | PC | [symbol] 0000 | 3333 | 1111 | 370 | [symbol] 23 | 4 [symbol] .2 | [symbol] 3.7 | 4. [symbol] | [symbol] .5 | 0.5 [symbol] | NC |
| | P | | | | | | | | | | | | |

[symbol]

indicates data missing or illegible when filed

### Day 2.

1. Test compounds were dissolved at 10 mM DMSO stock solution. 45 uL of stock solution was transferred to 384 pp-plate. A 3-fold, 10-point dilution was performed via transferring 15 uL compound into 30 uL DMSO by using TECAN liquid handler.
2. The plates were spinned at room temperature at 1,000 RPM for 1 minute.
3. 50 nL of diluted compound was transferred from compound source plate into the cell plate by Echo.
4. After compound treatment for 6 hours, the plate was removed from incubators and
5. The inhibition activity was calculated following the formula below:

$$\% Degrader = 100 \times ( LumHC - LumSample ) / ( ( LumHC - LumLC )$$

Note: Where HC was obtained from cells treated with 0.1% DM50 only; LC was obtained from culture medium only.
6. ABS DC₅₀ was calculated by filling the Curve using Xlfit (v5.3.1.3), equation 201:

$$Y = Bottom + ( Top - Bottom ) / ( 1 + 1 {0}^{∧} ( ( Log IC 50 - X ) * HillSlope ) )$$

The data from this assay is presented in Table 1, columns 7 and 8.

### HiBit Assay Protocol for CDK9

### 1. Materials

### 1.1 Reagents

| Reagents | Vendor | Cat# |
|---|---|---|
| HEK293-CDK9-Hibit cell | Pharmaron | \ |
| DMEM | Hyclone | SH30022 01 |
| FBS | Gibco | 10099141C |
| Nano-Glo ® HiBiT Lytic | Promega | N3040 |
| Detection System | | |
| 0.25% Trypsin/EDTA | Invitrogen | #25300 |
| PBS | Solarbio | P1020-500 |
| Penicillin-Streptomycin Liquid | Solarbio | P1400 |

| Instrument | Vendor | Cat# |
|---|---|---|
| Centrifuge | Eppendorf | 5810R |
| CO₂ Incubator | Thermo | Model: 371 |
| Vortex | IKA | MS3 digital |
| Microplate shaker | Yoning | WZ-4 |
| Echo Liquid Handler | Labcyte | 550 |
| TECAN | TECAN | Freedom EVO200 |
| 384 well plate | Corning | 3570 |
| 384 pp-plate | Labcyte | 001-14555 |
| 50 mL centrifuge tube | BD-Falcon | 352098 |
| 15 mL centrifuge tube | BD-Falcon | 352097 |
| EnVision | PerkinElmer | 2105-0020 |

### 2. Assay Procedure

### Day 1.

1. The culture medium for HEK293-CDK9-Hibit cell was prepared using DMEM with 10% FBS.
2. Cells were cultivated in T-75 flasks in a cell culture incubator set at 37° C., 5% CO2, 95% relative humidity. Cells were allowed to reach 80-90% confluence before detaching and splitting.
3. Cultivated cells were rinsed in T-75 flasks with 5 mL PBS. Aspirated off. Added 1.5 mL trypsin, and incubated the cells at 37° C. for approximately 5 minutes or until the cells detached and floated.
4. Trypsin was inactivated by adding excess serum containing medium.
5. Cells were harvested from flask into cell culture medium and then the cell number was counted. HEK293-CDK9-Hibit cells were seeded into 384-well plate (cell density: 1.6×105 cells/well/50 uL medium) in DMEM medium according to the plate map below and were incubated overnight at 37° C. and 5% CO2.
Note: Cell Culture Medium: 90% DMEM+10% FBS+1% Penicillin-Streptomycin Liquid

Plate map

| Top(nM) | | 1 | 2 | 3 | 4 | 5 | 6 | 7 | 8 | 9 | 10 | 11 | 12 |
|---|---|---|---|---|---|---|---|---|---|---|---|---|---|
| | A | | | | | | | | | | | | |
| [symbol] 0000 | B | NC | [symbol] 0000 | 3333 | 1111 | 370 | [symbol] 23 | 4 [symbol] .2 | [symbol] 3.7 | 4. [symbol] | [symbol] .5 | 0.5 [symbol] | PC |
| [symbol] 0000 | C | NC | [symbol] 0000 | 3333 | 1111 | 370 | [symbol] 23 | 4 [symbol] .2 | [symbol] 3.7 | 4. [symbol] | [symbol] .5 | 0.5 [symbol] | PC |
| [symbol] 0000 | D | NC | [symbol] 0000 | 3333 | 1111 | 370 | [symbol] 23 | 4 [symbol] .2 | [symbol] 3.7 | 4. [symbol] | [symbol] .5 | 0.5 [symbol] | PC |
| [symbol] 0000 | E | NC | [symbol] 0000 | 3333 | 1111 | 370 | [symbol] 23 | 4 [symbol] .2 | [symbol] 3.7 | 4. [symbol] | [symbol] .5 | 0.5 [symbol] | PC |
| [symbol] 0000 | F | NC | [symbol] 0000 | 3333 | 1111 | 370 | [symbol] 23 | 4 [symbol] .2 | [symbol] 3.7 | 4. [symbol] | [symbol] .5 | 0.5 [symbol] | PC |
| [symbol] 0000 | G | NC | [symbol] 0000 | 3333 | 1111 | 370 | [symbol] 23 | 4 [symbol] .2 | [symbol] 3.7 | 4. [symbol] | [symbol] .5 | 0.5 [symbol] | PC |
| [symbol] 0000 | H | NC | [symbol] 0000 | 3333 | 1111 | 370 | [symbol] 23 | 4 [symbol] .2 | [symbol] 3.7 | 4. [symbol] | [symbol] .5 | 0.5 [symbol] | PC |
| [symbol] 0000 | I | NC | [symbol] 0000 | 3333 | 1111 | 370 | [symbol] 23 | 4 [symbol] .2 | [symbol] 3.7 | 4. [symbol] | [symbol] .5 | 0.5 [symbol] | PC |
| [symbol] 0000 | J | NC | [symbol] 0000 | 3333 | 1111 | 370 | [symbol] 23 | 4 [symbol] .2 | [symbol] 3.7 | 4. [symbol] | [symbol] .5 | 0.5 [symbol] | PC |
| [symbol] 0000 | K | NC | [symbol] 0000 | 3333 | 1111 | 370 | [symbol] 23 | 4 [symbol] .2 | [symbol] 3.7 | 4. [symbol] | [symbol] .5 | 0.5 [symbol] | PC |
| [symbol] 0000 | L | NC | [symbol] 0000 | 3333 | 1111 | 370 | [symbol] 23 | 4 [symbol] .2 | [symbol] 3.7 | 4. [symbol] | [symbol] .5 | 0.5 [symbol] | PC |
| [symbol] 0000 | M | NC | [symbol] 0000 | 3333 | 1111 | 370 | [symbol] 23 | 4 [symbol] .2 | [symbol] 3.7 | 4. [symbol] | [symbol] .5 | 0.5 [symbol] | PC |
| [symbol] 0000 | N | NC | [symbol] 0000 | 3333 | 1111 | 370 | [symbol] 23 | 4 [symbol] .2 | [symbol] 3.7 | 4. [symbol] | [symbol] .5 | 0.5 [symbol] | PC |
| [symbol] 0000 | O | NC | [symbol] 0000 | 3333 | 1111 | 370 | [symbol] 23 | 4 [symbol] .2 | [symbol] 3.7 | 4. [symbol] | [symbol] .5 | 0.5 [symbol] | PC |
| | P | | | | | | | | | | | | |
| Top(nM) | | 13 | 14 | 15 | 16 | 17 | 18 | 19 | 20 | 21 | 22 | 23 | 24 |
| | A | | | | | | | | | | | | |
| [symbol] 0000 | B | PC | [symbol] 0000 | 3333 | 1111 | 370 | [symbol] 23 | 4 [symbol] .2 | [symbol] 3.7 | 4. [symbol] | [symbol] .5 | 0.5 [symbol] | NC |
| [symbol] 0000 | C | PC | [symbol] 0000 | 3333 | 1111 | 370 | [symbol] 23 | 4 [symbol] .2 | [symbol] 3.7 | 4. [symbol] | [symbol] .5 | 0.5 [symbol] | NC |
| [symbol] 0000 | D | PC | [symbol] 0000 | 3333 | 1111 | 370 | [symbol] 23 | 4 [symbol] .2 | [symbol] 3.7 | 4. [symbol] | [symbol] .5 | 0.5 [symbol] | NC |
| [symbol] 0000 | E | PC | [symbol] 0000 | 3333 | 1111 | 370 | [symbol] 23 | 4 [symbol] .2 | [symbol] 3.7 | 4. [symbol] | [symbol] .5 | 0.5 [symbol] | NC |
| [symbol] 0000 | F | PC | [symbol] 0000 | 3333 | 1111 | 370 | [symbol] 23 | 4 [symbol] .2 | [symbol] 3.7 | 4. [symbol] | [symbol] .5 | 0.5 [symbol] | NC |
| [symbol] 0000 | G | PC | [symbol] 0000 | 3333 | 1111 | 370 | [symbol] 23 | 4 [symbol] .2 | [symbol] 3.7 | 4. [symbol] | [symbol] .5 | 0.5 [symbol] | NC |
| [symbol] 0000 | H | PC | [symbol] 0000 | 3333 | 1111 | 370 | [symbol] 23 | 4 [symbol] .2 | [symbol] 3.7 | 4. [symbol] | [symbol] .5 | 0.5 [symbol] | NC |
| [symbol] 0000 | I | PC | [symbol] 0000 | 3333 | 1111 | 370 | [symbol] 23 | 4 [symbol] .2 | [symbol] 3.7 | 4. [symbol] | [symbol] .5 | 0.5 [symbol] | NC |
| [symbol] 0000 | J | PC | [symbol] 0000 | 3333 | 1111 | 370 | [symbol] 23 | 4 [symbol] .2 | [symbol] 3.7 | 4. [symbol] | [symbol] .5 | 0.5 [symbol] | NC |
| [symbol] 0000 | K | PC | [symbol] 0000 | 3333 | 1111 | 370 | [symbol] 23 | 4 [symbol] .2 | [symbol] 3.7 | 4. [symbol] | [symbol] .5 | 0.5 [symbol] | NC |
| [symbol] 0000 | L | PC | [symbol] 0000 | 3333 | 1111 | 370 | [symbol] 23 | 4 [symbol] .2 | [symbol] 3.7 | 4. [symbol] | [symbol] .5 | 0.5 [symbol] | NC |
| [symbol] 0000 | M | PC | [symbol] 0000 | 3333 | 1111 | 370 | [symbol] 23 | 4 [symbol] .2 | [symbol] 3.7 | 4. [symbol] | [symbol] .5 | 0.5 [symbol] | NC |
| [symbol] 0000 | N | PC | [symbol] 0000 | 3333 | 1111 | 370 | [symbol] 23 | 4 [symbol] .2 | [symbol] 3.7 | 4. [symbol] | [symbol] .5 | 0.5 [symbol] | NC |
| [symbol] 0000 | O | PC | [symbol] 0000 | 3333 | 1111 | 370 | [symbol] 23 | 4 [symbol] .2 | [symbol] 3.7 | 4. [symbol] | [symbol] .5 | 0.5 [symbol] | NC |
| | P | | | | | | | | | | | | |

[symbol]

indicates data missing or illegible when filed

### Day2.

1. Test compounds were dissolved at 3 mM DMSO stock solution. Transferred 45 μL of stock solution to a 384 pp-plate. Perform 3-fold, 10-point dilution via transferring 15 uL compound into 30 uL DMSO by using TECAN liquid handler.
2. The plates were spinned at room temperature at 1,000 RPM for 1 minute.
3. Transferred 50 nL of diluted compound from compound source plate into the cell plate by Echo.
4. After compound treatment for 6 hours, removed the plate from incubators and equilibrated at room temperature for 15 minutes. Prepared the HiBit reagent consists of the following ratios: 10 mL of Nano-Glo®-HiBiT Lytic Buffer, 200 μL of Nano-Glo®-HiBiT Lytic Substrate and 100 uL ofLgBiT Protein, mixed thoroughly. Added 20 uL ofHiBit reagent into each well and spinned the plates at room temperature at 1,000 RPM for 1 minute to be detected. Plates were then shaken at 600 RPM at room temperature for 20 minutes, and read by EnVision.
5. The inhibition activity was calculated following the formula below:

$$\% Degrader = 100 \times ( LumHC - LumSample ) / ( ( LumHC - LumLC )$$

Note: Where HC was obtained from cells treated with 0.1% DMSO only; LC was obtained from culture medium only.
6. The ABS DC50 was calculated by fitting the Curve using Xlfit (v5.3.1.3), equation 201:

$$Y = Bottom + ( Top - Bottom ) / ( 1 + 1 {0}^{∧} ( ( Log IC 50 - X ) * HillSlope ) )$$

The data from this assay is presented in Table 1, columns 5 and 6.

1. A compound of Formula A-I: 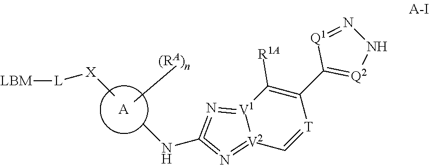
or a pharmaceutically acceptable salt thereof, wherein: [symbol]
s a single or a double bond;
Ring A is selected from the group consisting of a nitrogen-containing 4-10 member heterocyclyl, a C₆₋₁₀ aryl and a 5-10-member heteroaryl, wherein the 4-10 member heterocyclyl, C₆₋₁₀ aryl and 5-10-member heteroaryl are attached to the —NH— through a carbon atom;
V¹ is nitrogen and V² is carbon, and Ring 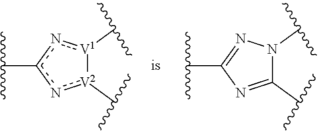
or V² is nitrogen and V¹ is carbon, and Ring 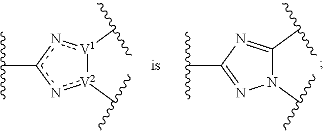
T is CH or N;
Q¹ and Q² are independently selected from N and CH;
R^{1A} is independently selected from H, D, halo, CN, NO₂, C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocyclyl, 5-10 membered heteroaryl, C₃₋₁₀ cycloalkyl-C₁₋₄ alkyl, 6-10 membered aryl-C₁₋₄ alkyl-, 4-10 membered heterocyclyl-C₁₋₄ alkyl-, 5-10 membered heteroaryl-C₁₋₄ alkyl-, OR^{a1}, SR^{a1} NHOR^{a1}, C(O)R^{b1}, C(O)NR^{a1}R^{a1}, C(O)NR^{a1}(OR^{a1}), C(O)OR^{a1}, OC(O)R^{b1}, OC(O)NR^{a1}R^{a1}, NR^{a1}R^{a1}, NR^{a1}NR^{a1}R^{a1}, NR^{a1}C(O)R^{b1}, NR^{a1}C(O)OR^{a1}, NR^{a1}C(O)NR^{a1}R^{a1}, C(═NR^{a1})R^{b1}, C(═NR^{a1})NR^{a1}R^{a1}, NR^{a1}C(═NR^{a1})NR^{a1}R^{a1}, NR^{a1}C(═NR^{a1})R^{b1}, NR^{a1}S(O)NR^{a1}R^{a1}, NR^{a1}S(O)R^{b1}, NR^{a1}S(O)₂R^{b1}, NR^{a1}S(O)(═NR^{a1})R^{b1}, NR^{a1}S(O)₂NR^{a1}R^{a1}, S(O)R^{b1}, S(O)NR^{a1}R^{a1}, S(O)₂R^{b1}, S(O)₂NR^{a1}R^{a1}, OS(O)(═NR^{a1})R^{b1}, OS(O)₂R^{b1}, S(O)(═NR^{a1})R^{b1}, SF₅, P(O)R^{a1}R^{b1}, OP(O)(OR^{a1})(OR^{a1}) and P(O)(OR^{a1})(OR^{a1}), wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocyclyl, 5-10 membered heteroaryl, C₃₋₁₀ cycloalkyl-C₁₋₄ alkyl-, 6-10 membered aryl-C₁₋₄ alkyl-, 4-10 membered heterocyclyl-C₁₋₄ alkyl-, and 5-10 membered heteroaryl-C₁₋₄ alkyl-are each substituted with 0, 1, 2, 3, or 4 independently selected R² substituents;
each R² is independently selected from H, D, halo, CN, NO₂, C₁₋₆ alkyl, C₂₋₆ alkenyl, C₂₋₆ alkynyl, C₁₋₆ haloalkyl, C₃₋₇ cycloalkyl, phenyl, 4-7 membered heterocyclyl, 5-6 membered heteroaryl, C₃₋₇ cycloalkyl-C₁₋₄ alkyl-, phenyl-C₁₋₄ alkyl-, 4-7 membered heterocyclyl-C₁₋₄ alkyl-, 5-6 membered heteroaryl-C₁₋₄ alkyl-, OR^{a2}, SR^{a2}, NHOR^{a2}, C(O)R^{b2}, C(O)NR^{a2}R^{a2}, C(O)NR^{a2}(OR^{a2}), C(O)OR^{a2}, OC(O)R^{b2}, OC(O)NR^{a2}R^{a2}, NR^{a2}R^{a2}, NR^{a2}NR^{a2}R^{a2}, NR^{a2}C(O)R^{b2}, NR^{a2}C(O)OR^{a2}, NR^{a2}C(O)NR^{a2}R^{a2}, C(═NR^{a2})R^{b2}, C(═NR^{a2})NR^{a2}R^{a2}, NR^{a2}C(═NR^{a2})NR^{a2}R^{a2}, NR^{a2}C(═NR^{a2})R^{b2}, NR^{a2}S(O)NR^{a2}R^{a2}, NR^{a2}S(O)R^{b2}, NR^{a2}S(O)₂R^{b2}, NR^{a2}S(O)(═NR^{a2})R^{b2}, NR^{a2}S(O)₂NR^{a2}R^{a2}, S(O)R^{b2}, S(O)NR^{a2}R^{a2}, S(O)₂R^{b2}, S(O)₂NR^{a2}R^{a2}, OS(O)(═NR^{a2})R^{b2}, OS(O)₂R^{b2}, S(O)(═NR^{a2})R^{b2}, SF₅, P(O)R^{a2}R^{a2}, OP(O)(OR^{a2})(OR^{a2}) and P(O)(OR^{a2})(OR^{a2}), wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocyclyl, 5-6 membered heteroaryl, C₃₋₇ cycloalkyl-C₁₋₄ alkyl-, phenyl-C₁₋₄ alkyl, 4-7 membered heterocyclyl-C₁₋₄ alkyl-, and 5-6 membered heteroaryl-C₁₋₄ alkyl-are each substituted with 0, 1, 2, 3, or 4 substituents independently selected from C₁₋₄ alkyl, C₃₋₇ cycloalkyl, cyclopropyl, oxo, —C(O)C₁₋₄alkyl, —C(O)OC₁₋₄alkyl, —C(O)NH₂, —OH, —F, —Cl, —O—C₁₋₄alkyl and —CN;
each instance of R^{A} is independently selected from —D, halo, CN, C₁₋₄ alkyl, C₁₋₄ haloalkyl, C₃₋₆ cycloalkyl, 4-6 membered heterocyclyl, OR^{a1}, SR^{a1}, SF₅, NR^{a1}R^{a1}, C₃₋₆ cycloalkyl-C₁₋₃ alkyl-, and 4-6 membered heterocyclyl-C₁₋₃ alkyl-, wherein said C₁-4 alkyl, C₁₋₄haloalkyl-, C₃₋₆ cycloalkyl, 4-6 membered heterocyclyl-, C₃₋₆ cycloalkyl-C₁₋₃ alkyl-, and 4-6 membered heterocyclyl-C₁₋₃ alkyl are substituted with 0, 1, 2, 3, or 4 substituents independently selected from —D, halo, —OH, —C₁₋₄ alkyl and —OC₁₋₄ alkyl;
or, alternatively, two R^{A} groups on adjacent atoms of Ring A, together with the ring atoms to which they are attached, form Ring D, wherein Ring D is selected from C₃₋₆ cycloalkyl, 4-6 membered heterocyclyl, phenyl, and 5-6 membered heteroaryl, each of which is substituted with 0, 1, 2, 3, or 4 substituents independently selected from —D, halo, —OH, —C₁₋₄ alkyl and —OC₁₋₄ alkyl;
each R^{a1} is independently selected from H, C₁₋₆ alkyl, C₁₋₆ haloalkyl, C₂₋₆ alkenyl, C₂₋₆ alkynyl, C₃₋₁₀ cycloalkyl, 6-10 membered aryl, 4-10 membered heterocyclyl, 5-10 membered heteroaryl, C₃₋₁₀ cycloalkyl-C₁₋₄ alkyl-, 6-10 membered aryl-C₁₋₄ alkyl-, 4-10 membered heterocyclyl-C₁₋₄ alkyl-, and 5-10 membered heteroaryl-C₁₋₄ alkyl-, wherein said C₁₋₆ alkyl, C₂₋₆ alkenyl, C₂₋₆ alkynyl, C₁₋₆ haloalkyl, C₃₋₁₀ cycloalkyl, 6-10 membered aryl, 4-10 membered heterocyclyl, 5-10 membered heteroaryl, C₃₋₁₀ cycloalkyl-C₁₋₄ alkyl, 6-10 membered aryl-C₁₋₄ alkyl, 4-10 membered heterocyclyl-C₁₋₄ alkyl, and 5-10 membered heteroaryl-C₁₋₄ alkyl are each substituted with 0, 1, 2, 3, or 4 independently selected R² substituents; or two R^{a1} groups attached to the same nitrogen atom together with the nitrogen to which they are attached form a 4-7-membered heterocyclyl group substituted with 0, 1, 2, 3, or 4 substituents independently selected from —D, halo, —OH, —C₁₋₄ alkyl and —OC₁₋₄ alkyl;
each R^{b1} is independently selected from C₁₋₆ alkyl, C₁₋₆ hydroxyalkyl, C₃₋₉ cycloalkyl and C₂₋₆ heteroalkyl;
each R^{a2} is independently selected from H, C₁₋₆ alkyl, C₁₋₆ hydroxyalkyl, C₃₋₉ cycloalkyl and C₂₋₆ heteroalkyl, or, when possible, two instances of R^{a2} and the atom to which they are attached are taken together to form a 4-7 member heterocycle substituted with 0, 1, 2, 3, or 4 substituents independently selected from —D, halo, —OH, —C₁₋₄ alkyl and —OC₁-4 alkyl;
each R^{b2} is independently selected from C₁₋₆ alkyl, C₁₋₆ hydroxyalkyl, C₃₋₉ cycloalkyl and C₂₋₆ heteroalkyl;
X is X¹ when Ring A is heterocyclyl and is selected from X¹ and X² when Ring A is aryl or heteroaryl;
X¹ is selected from —S(O)₂— and —C(O)—;
X² is selected from —O—, —NH—, —N(CH₃)— and —CH₂—;
L is a covalent bond or a bivalent, saturated or unsaturated, straight or branched C₁₋₅₀ hydrocarbon chain, wherein 0-6 methylene units of L are independently replaced by —Cy—, —CH(R)—, —C(R)₂—, —O—, —NR—, —S—, —OC(═O)—, —C(═O)O—, —C(═O)—, —S(═O)—, —S(═O)₂—, —NRS(═O)₂—, —S(═O)₂NR—, —NRC(═O)—, —C(═O)NR—, —OC(═O)NR— or —NRC(═O)O—, wherein:
each —Cy— is independently a bivalent ring selected from phenylene, an 8-10 membered bicyclic arylene, a 4-7 membered monocyclic carbocyclylene, a 5-11 membered Spiro carbocyclylene, a 4-10 membered bicyclic carbocyclylene, a 5-10 membered bridged carbocyclylene, a 4-7 membered monocyclic heterocyclylene having 1-2 heteroatoms independently selected from nitrogen, oxygen, and sulfur, a 5-11 membered spiro heterocyclylene having 1-2 heteroatoms independently selected from nitrogen, oxygen, and sulfur, an 4-10 membered bicyclic heterocyclylene having 1-2 heteroatoms independently selected from nitrogen, oxygen, and sulfur, a 5-10 membered bridged bicyclic saturated or partially unsaturated heterocyclylene having 1-2 heteroatoms independently selected from nitrogen, oxygen, and sulfur, a 5-6 membered heteroarylene having 1-4 heteroatoms independently selected from nitrogen, oxygen, and sulfur, or an 8-10 membered bicyclic heteroarylene having 1-5 heteroatoms independently selected from nitrogen, oxygen, or sulfur, wherein each phenylene, arylene, carbocyclylene, heterocyclylene and heteroarylene is substituted with 0, 1, 2, 3, or 4 instances of R^{C};
LBM is selected from: 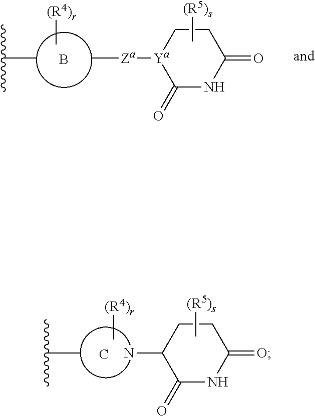
Y^{a} is CH or N;
Z^{a} is a bond, —CH₂—, —NH—, O, or —NHC(O)— where NH of —NHC(O)— is attached to Y^{a};
Ring B is phenylene, a 4-10-membered heterocyclylene, a 5-6-membered monocyclic heteroarylene or a 9-10-membered fused bicyclic heteroarylene, wherein each heteroarylene contains one to three nitrogen ring atoms.
ring C together with the (R⁴)r substituents is selected from the group consisting of: 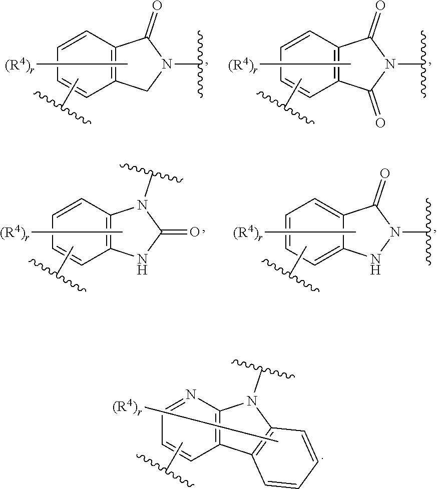
each instance of Re is independently selected from —D, halogen, —OH, and —C₁₋₆ alkyl;
each instance of R⁴ is independently selected from —D, halogen, —OH, and —C₁₋₆ alkyl;
each instance of R⁵ is independently selected from —D, halogen, —OH, and —C₁₋₆ alkyl;
each instance of R is independently selected from hydrogen and —C₁₋₆ alkyl;
n is 0, 1, 2, 3, or 4;
r is 0, 1, 2, 3, or 4; and
s is 0, 1, 2, 3, or 4.

2. A compound of Formula I: 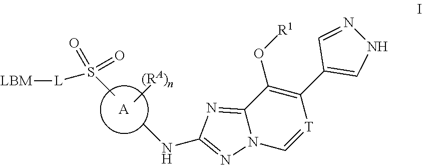
or a pharmaceutically acceptable salt thereof, wherein:
Ring A is selected from 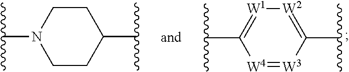
each W¹, W², W³ and W⁴ is independently selected from CH and N, provided that no more than 2 of W¹, W², W³ and W⁴ are N;
T is CH or N;
R¹ is independently selected from H, C₁₋₆ alkyl, C₁₋₆ haloalkyl, C₃₋₁₀ cycloalkyl, 4-10 membered heterocyclyl, C₃₋₁₀ cycloalkyl-C₁₋₄ alkyl- and 4-10 membered heterocyclyl-C₁₋₄ alkyl-, wherein said C₁₋₆ alkyl, C₁₋₆ haloalkyl, C₃₋₁₀ cycloalkyl, 4-10 membered heterocyclyl, C₃₋₁₀ cycloalkyl-C₁₋₄ alkyl- and 4-10 membered heterocyclyl-C₁₋₄ alkyl-, are each substituted with 0, 1, 2, 3, or 4 independently selected R² substituents;
each R² is independently selected from D, halo, CN, C₁₋₆ alkyl, C₁₋₆ haloalkyl, C₃₋₇ cycloalkyl, OR^{a2}, C(O)R^{b2}, C(O)NR^{a2}R^{a2}, C(O)OR^{a2}, OC(O)R^{b2}, OC(O)NR^{a2}R^{a2}, NR^{a2}R^{a2}, NR^{a2}C(O)R^{b2}, NR^{a2}C(O)OR^{a2}, NR^{a2}C(O)NR^{a2}R^{a2}, NR^{a2}S(O)NR^{a2}R^{a2}, NR^{a2}S(O)R^{b2}, NR^{a2}S(O)₂R^{b2}, NR^{a2}S(O)₂NR^{a2}R^{a2}, S(O)R^{b2}, S(O)NR^{a2}R^{a2}, S(O)₂R^{b2}, S(O)₂NR^{a2}R^{a2}, SF₅, wherein said C₁₋₆ alkyl, C₁₋₆ haloalkyl, C₃₋₇ cycloalkyl and 4-7 membered heterocyclyl are each optionally substituted with 1, 2, 3, or 4 substituents independently selected from C₁₋₄ alkyl, C₃₋₇ cycloalkyl, cyclopropyl, oxo, —C(O)C₁-4alkyl, —C(O)OC₁₋₄alkyl, —C(O)NH₂, —OH, —F, —Cl, —O—C₁-4alkyl and —CN;
each R^{a2} is independently selected from H, C₁₋₆ alkyl, C₁₋₆ hydroxyalkyl, C₃₋₉ cycloalkyl and C₂₋₆ heteroalkyl, or, when possible, two instances of R^{a2} and the atom to which they are attached are taken together to form a saturated 3-7-membered heterocycle;
each R^{b2} is independently selected from C₁₋₆ alkyl, C₁₋₆ hydroxyalkyl, C₃₋₉ cycloalkyl and C₂₋₆ heteroalkyl;
L is a covalent bond or a bivalent, saturated or unsaturated, straight or branched C₁₋₅₀ hydrocarbon chain, wherein 0-6 methylene units of L are independently replaced by —Cy—, —CH(R)—, —C(R)₂—, —O—, —NR—, —S—, —OC(═O)—, —C(═O)O—, —C(═O)—, —S(═O)—, —S(═O)₂—, —NRS(═O)₂—, —S(═O)₂NR—, —NRC(═O)—, —C(═O)NR—, —OC(═O)NR— or —NRC(═O)O—, wherein:
each —Cy— is independently a bivalent ring selected from phenylene, an 8-10 membered bicyclic arylene, a 4-7 membered monocyclic carbocyclylene, a 5-11 membered spiro carbocyclylene, a 4-10 membered bicyclic carbocyclylene, a 5-10 membered bridged carbocyclylene, a 4-7 membered monocyclic heterocyclylene having 1-2 heteroatoms independently selected from nitrogen, oxygen, and sulfur, a 5-11 membered spiro heterocyclylene having 1-2 heteroatoms independently selected from nitrogen, oxygen, and sulfur, an 4-10 membered bicyclic heterocyclylene having 1-2 heteroatoms independently selected from nitrogen, oxygen, and sulfur, a 5-10 membered bridged bicyclic saturated or partially unsaturated heterocyclylene having 1-2 heteroatoms independently selected from nitrogen, oxygen, and sulfur, a 5-6 membered heteroarylene having 1-4 heteroatoms independently selected from nitrogen, oxygen, and sulfur, or an 8-10 membered bicyclic heteroarylene having 1-5 heteroatoms independently selected from nitrogen, oxygen, or sulfur, wherein each phenylene, arylene, carbocyclylene, heterocyclylene and heteroarylene is substituted with 0, 1, 2, 3, or 4 instances of R^{C};
LBM is selected from 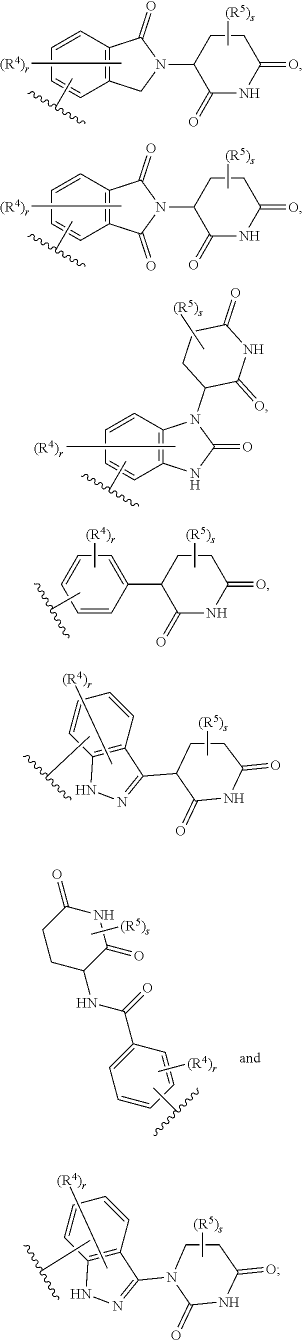
each instance of R^{A} is independently selected from —D, halogen, —C₁₋₆ alkyl, —OH and —OC₁₋₄ alkyl, wherein each —C₁₋₆ alkyl is substituted with 0, 1, 2 or 3 groups independently selected from —D, —F, —OH and —OC₁₋₄ alkyl or two RA groups on adjacent atoms of Ring A, together with the ring atoms to which they are attached, form Ring D, wherein Ring D is selected from C₃₋₆ cycloalkyl, 4-6 membered heterocyclyl, phenyl, and 5-6 membered heteroaryl, each of which is substituted with 0, 1, 2, 3, or 4 substituents independently selected from —D, halo, —OH, —C₁₋₄ alkyl and —OC₁₋₄ alkyl;
each instance of R^{C} is independently selected from —D, halogen, —OH, and —C₁₋₆ alkyl;
each instance of R⁴ is independently selected from —D, halogen, —OH, and —C₁₋₆ alkyl;
each instance of R⁵ is independently selected from —D, halogen, —OH, and —C₁₋₆ alkyl;
each instance of R is independently selected from hydrogen and —C₁₋₆ alkyl;
n is 0, 1, 2, 3, or 4;
r is 0, 1, 2, 3, or 4; and
s is 0, 1, 2, 3, or 4.

3. (canceled)

4. (canceled)

5. The compound of claim 2, or a pharmaceutically acceptable salt thereof, wherein the compound is a compound of Formula II-1

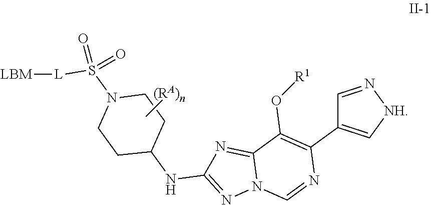

6. The compound of claim 2, or a pharmaceutically acceptable salt thereof, wherein the compound is a compound of Formula III-1

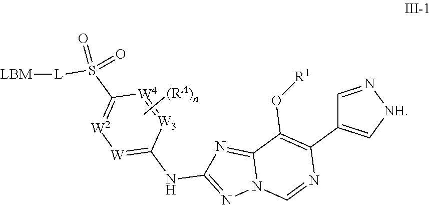

7. (canceled)

8. The compound of claim 2, or a pharmaceutically acceptable salt thereof, wherein R¹ is selected from C₁₋₆ alkyl, C₁₋₆ haloalkyl and C₃₋₆ cycloalkyl, wherein said C₁₋₆ alkyl, C₁₋₆ haloalkyl and C₃₋₆ cycloalkyl are each substituted with 0, 1 or 2 independently selected R² substituents.

9. (canceled)

10. (canceled)

11. (canceled)

12. The compound of claim 1, or a pharmaceutically acceptable salt thereof, wherein each R^{A} is independently selected from —D, halogen, —C₁₋₆ alkyl, —OH OCF₃, —OCHF₂, and —OC₁₋₄ alkyl, wherein each —C₁₋₆ alkyl is substituted with 0, 1, 2 or 3 groups independently selected from —D, —F, —OH and —OC₁₋₄ alkyl or two R^{A} are taken together with the atoms to which they are attached to form an aryl or heteroaryl.

13. (canceled)

14. (canceled)

15. (canceled)

16. (canceled)

17. (canceled)

18. (canceled)

19. (canceled)

20. (canceled)

21. The compound of claim 1, or a pharmaceutically acceptable salt thereof, wherein n is 0, 1 or 2.

22. (canceled)

23. (canceled)

24. (canceled)

25. (canceled)

26. The compound of claim 1, or a pharmaceutically acceptable salt thereof, wherein the moiety represented by 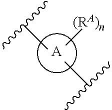
is selected from NN IF 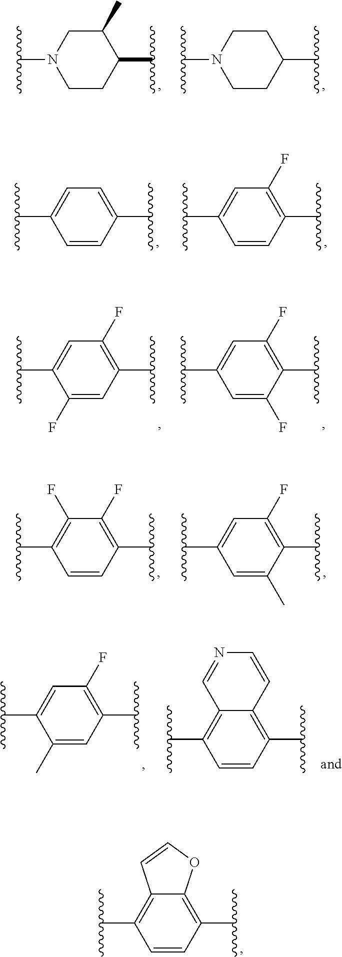
wherein the left attachment point connects to the —X— group of Formula A-I or the —S(O)₂— group of formula I and the right attachment point connects to the —NH— group of Formula A-I and formula I.

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. The compound of claim 1, or a pharmaceutically acceptable salt thereof, wherein the compound is of Formula IV.

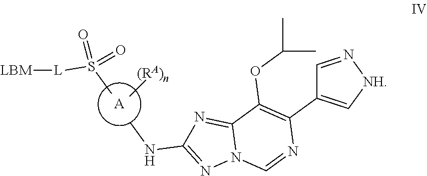

39. The compound of claim 1, or a pharmaceutically acceptable salt thereof, wherein the compound is of Formula IV-a.

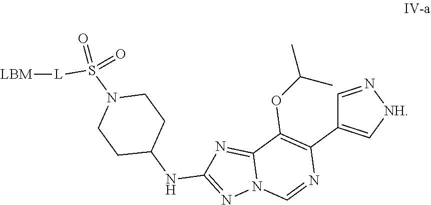

40. The compound of claim 1, or a pharmaceutically acceptable salt thereof, wherein the compound is of Formula IV-b.

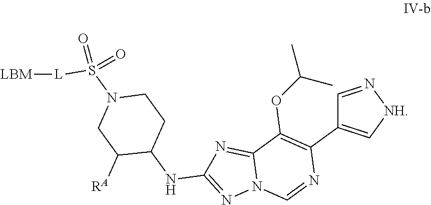

41. (canceled)

42. (canceled)

43. The compound of claim 1, or a pharmaceutically acceptable salt thereof, wherein the compound is of Formula IV-e

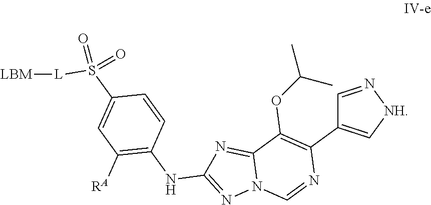

44. (canceled)

45. (canceled)

46. (canceled)

47. The compound of claim 1, or a pharmaceutically acceptable salt thereof, wherein the compound is of Formula IV-i:

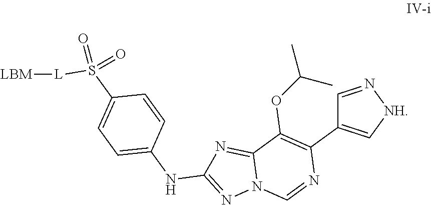

48. The compound of claim 1, or a pharmaceutically acceptable salt thereof, wherein L is selected from 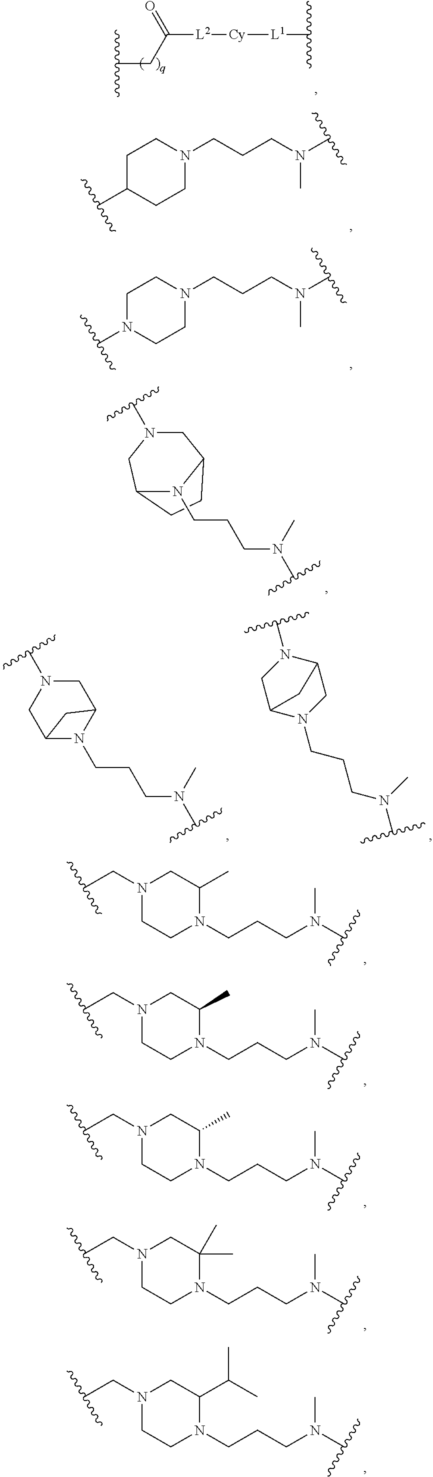 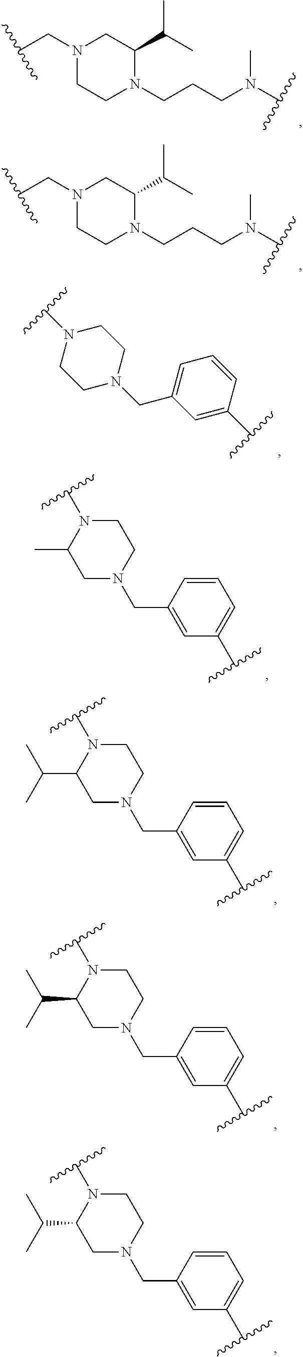 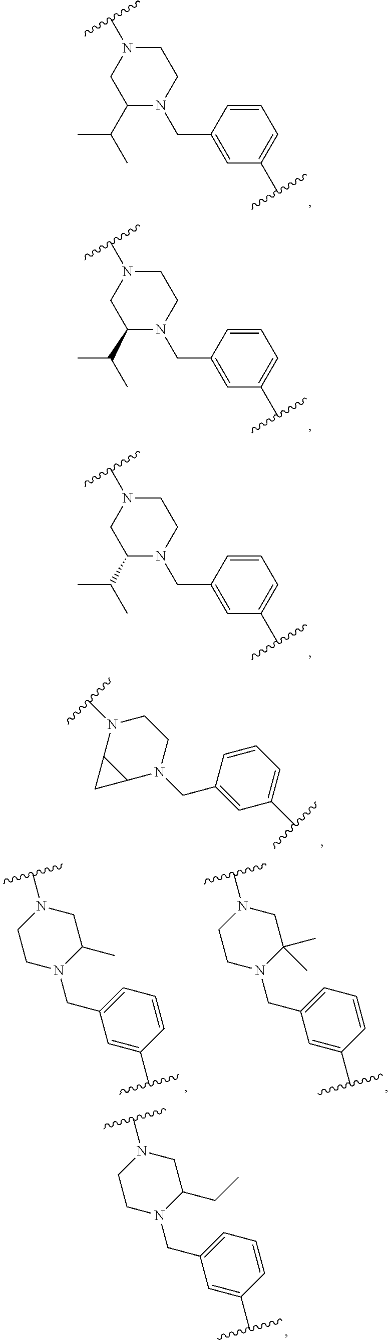 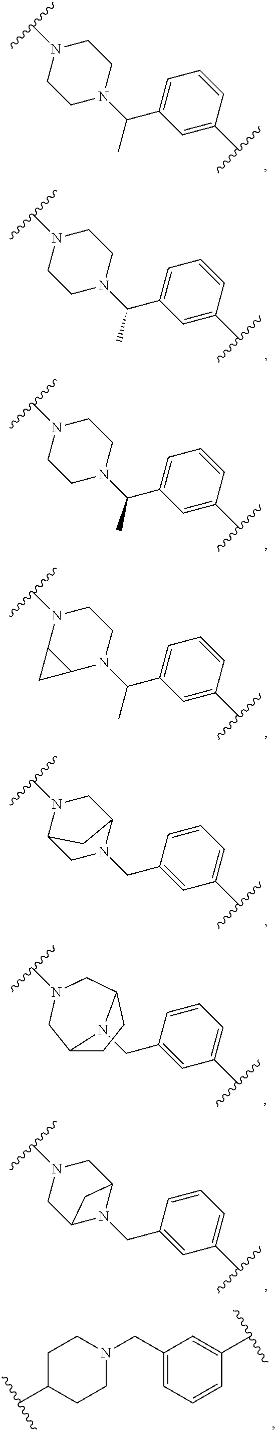 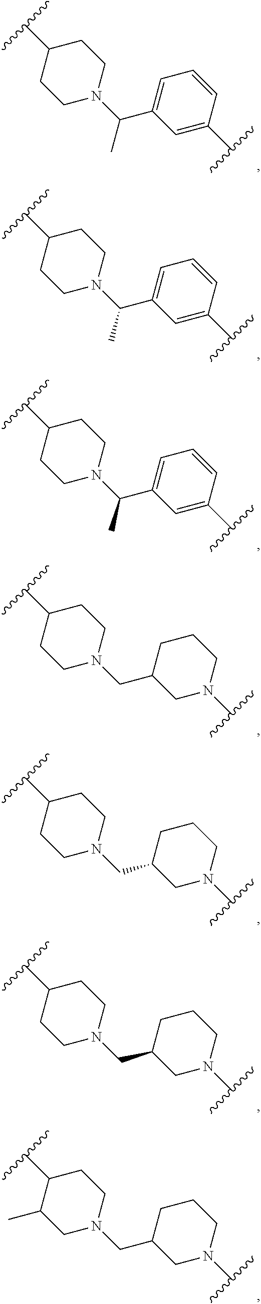 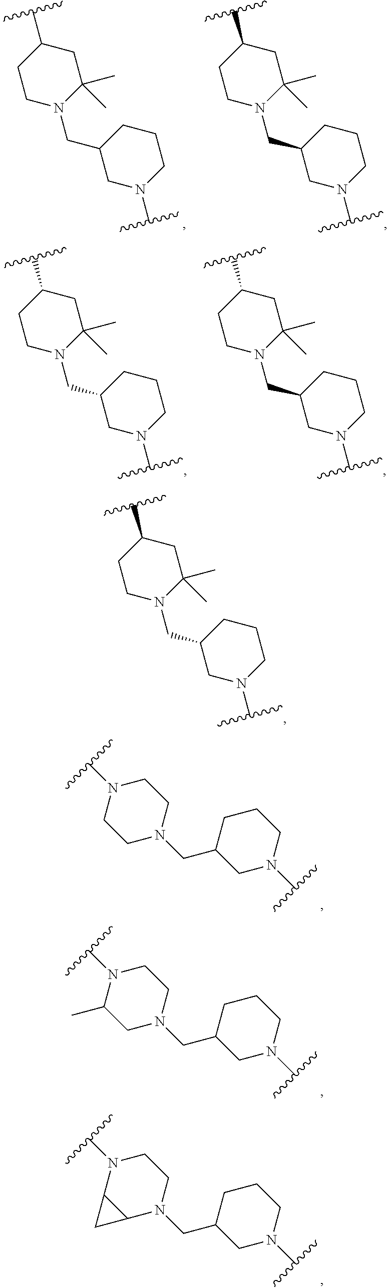 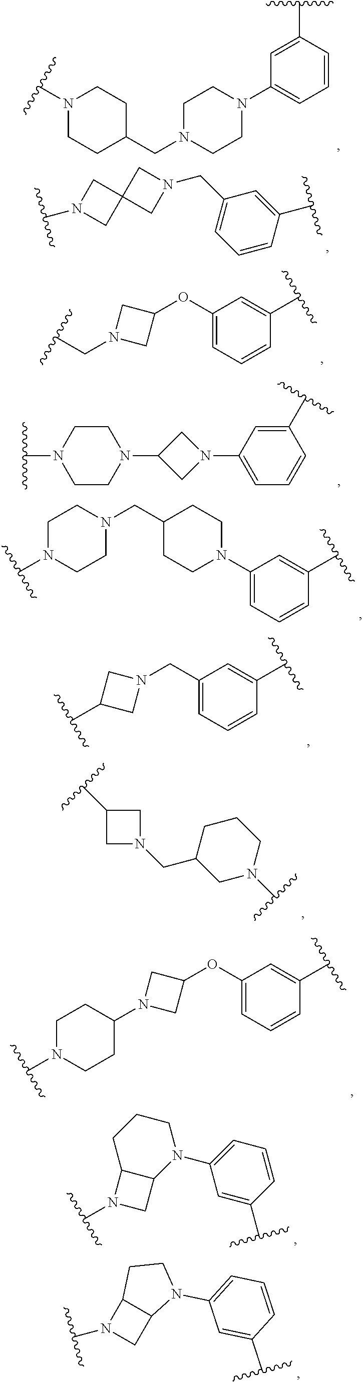 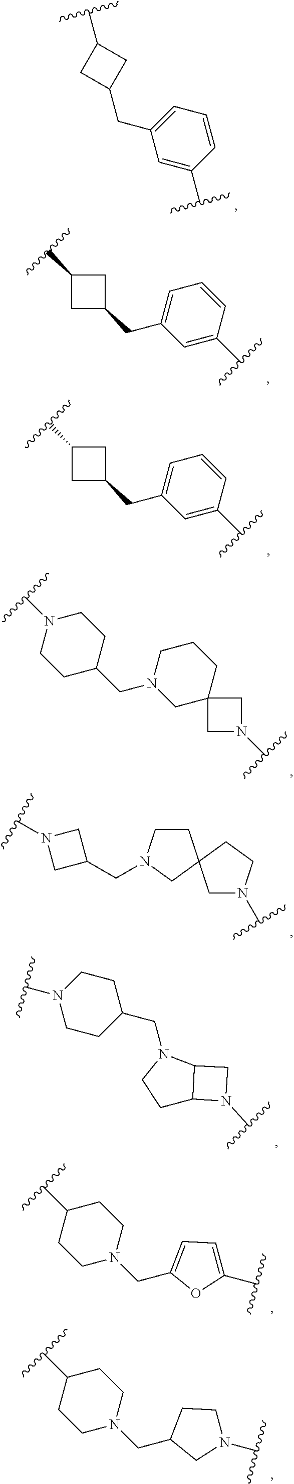 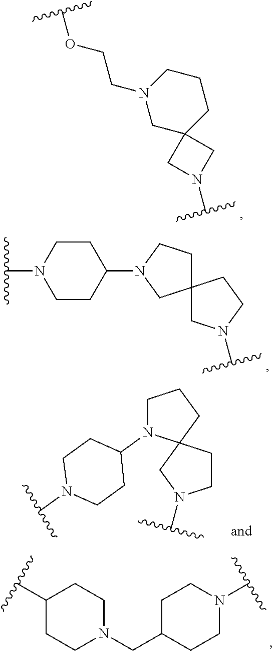
each substituted with 0, 1, 2 or 3 instances of R⁷, wherein each R⁷ is independently selected from —C₁₋₄ alkyl and halo;
wherein the left attachment point connects to LBM and the right attachment point connects to the —X— group of Formula A-I or —S(O)₂— group of Formula I and wherein
L¹ and L² are each independently selected from a bond and —N(R′), wherein R′ is selected from H and C₁₋₆ alkyl; and
q is 0, 1, 2, 3, 4, 5, 6, 7, 8, 9 or 10.

49. (canceled)

50. (canceled)

51. (canceled)

52. The compound of claim 1, or a pharmaceutically acceptable salt thereof, wherein each R⁴ is independently selected from —D, —Me, —Et, —F, —Cl and —OH.

53. (canceled)

54. The compound of claim 1, or a pharmaceutically acceptable salt thereof, wherein each R⁵ is independently selected from —Me, —Et, —F, —Cl and —OH.

55. (canceled)

56. The compound of claim 1, or a pharmaceutically acceptable salt thereof, wherein r is 0, 1 or 2, and wherein s is 0, 1 or 2.

57. (canceled)

58. (canceled)

59. (canceled)

60. (canceled)

61. (canceled)

62. (canceled)

63. (canceled)

64. The compound of claim 1, or a pharmaceutically acceptable salt thereof, wherein LBM is selected from:

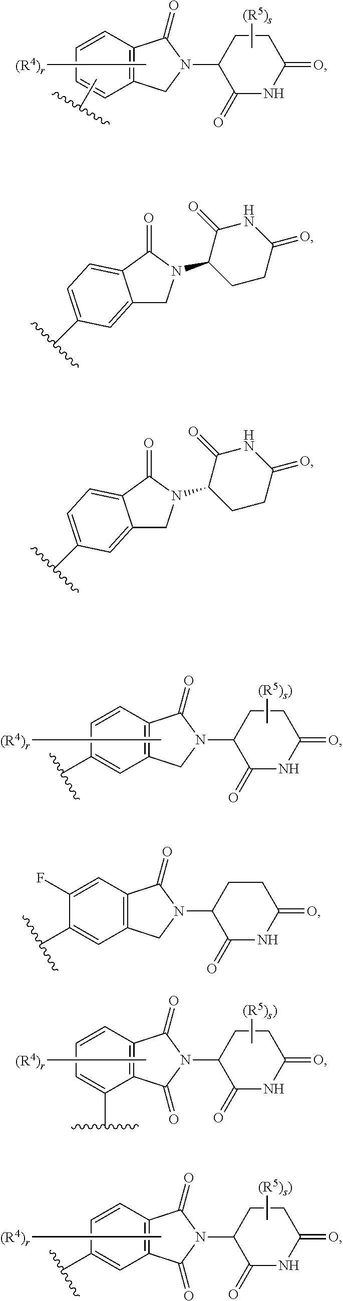

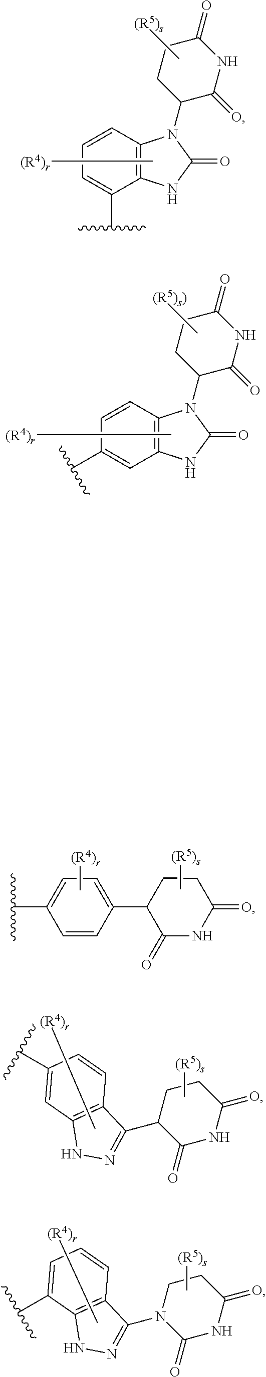

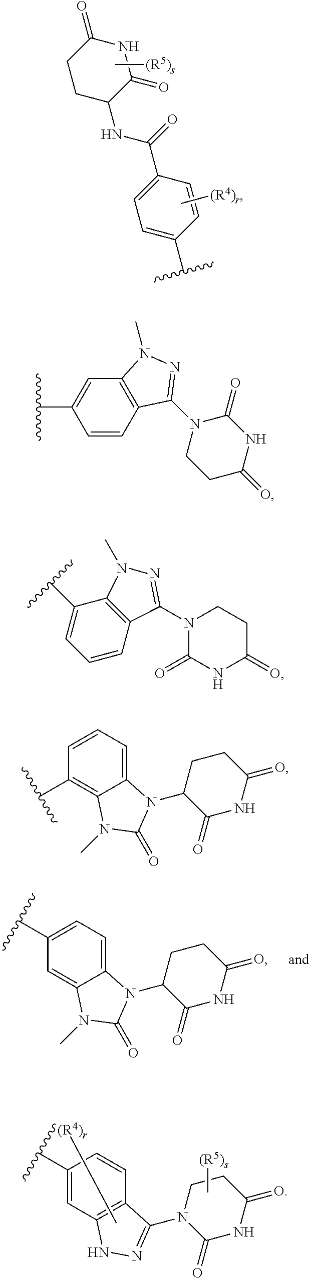

65. (canceled)

66. (canceled)

67. The compound of claim 1, or a pharmaceutically acceptable salt thereof, wherein the compound is selected from:

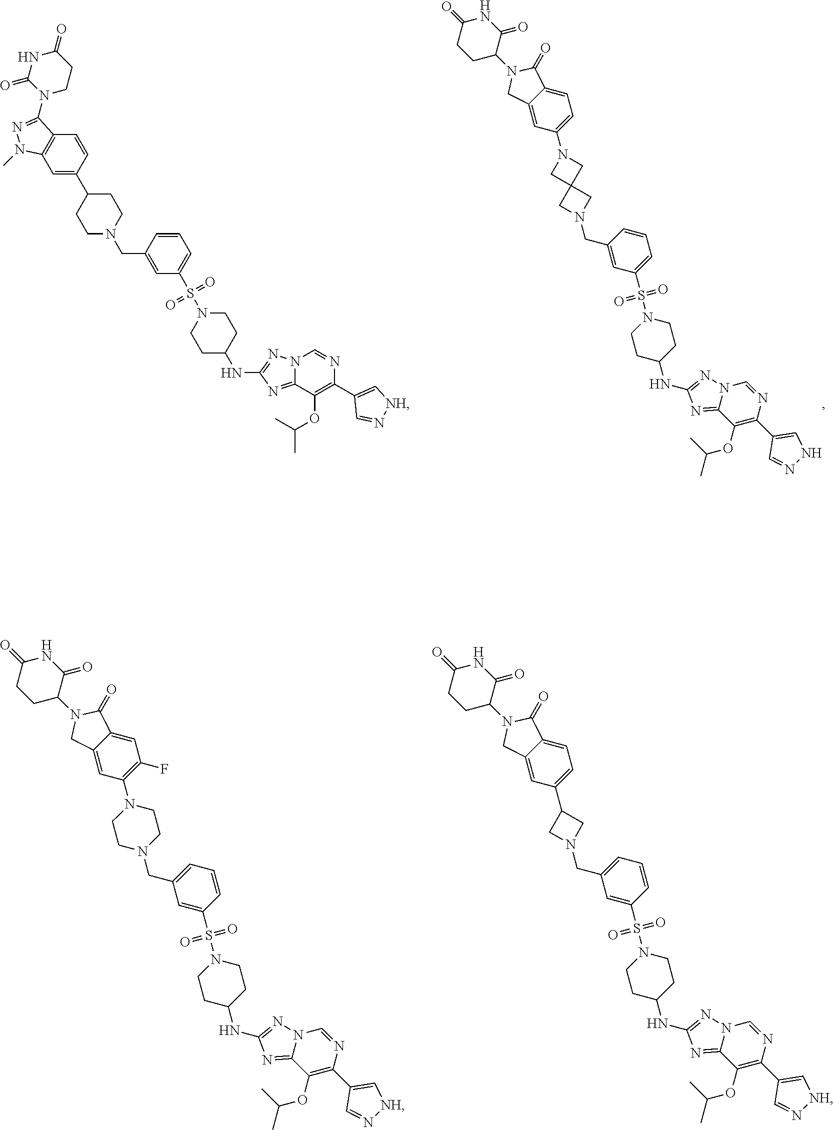 [SMILES: CC(C)Oc1c(-c2cn[nH]c2)ncn2nc(NC3CCN(S(=O)(=O)c4cccc(CN5CCC(c6ccc7c(N8CCC(=O)NC8=O)nn(C)c7c6)CC5)c4)CC3)nc12 ; CC(C)Oc1c(-c2cn[nH]c2)ncn2nc(NC3CCN(S(=O)(=O)c4cccc(CN5CC6(C5)CN(c5ccc7c(c5)CN(C5CCC(=O)NC5=O)C7=O)C6)c4)CC3)nc12 ; CC(C)Oc1c(-c2cn[nH]c2)ncn2nc(NC3CCN(S(=O)(=O)c4cccc(CN5CCN(c6cc7c(cc6F)C(=O)N(C6CCC(=O)NC6=O)C7)CC5)c4)CC3)nc12 ; CC(C)Oc1c(-c2cn[nH]c2)ncn2nc(NC3CCN(S(=O)(=O)c4cccc(CN5CC(c6ccc7c(c6)CN(C6CCC(=O)NC6=O)C7=O)C5)c4)CC3)nc12]

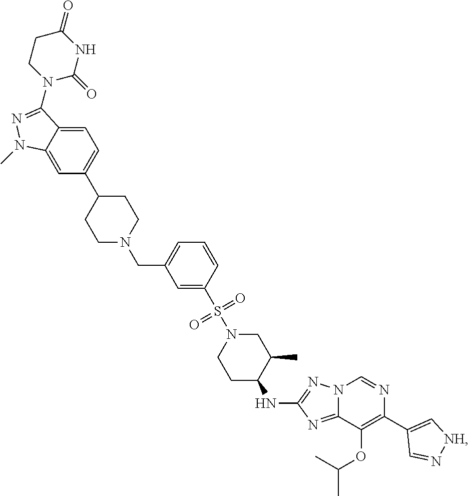 [SMILES: CC(C)Oc1c(-c2cn[nH]c2)ncn2nc(N[C@H]3CCN(S(=O)(=O)c4cccc(CN5CCC(c6ccc7c(N8CCC(=O)NC8=O)nn(C)c7c6)CC5)c4)C[C@H]3C)nc12]

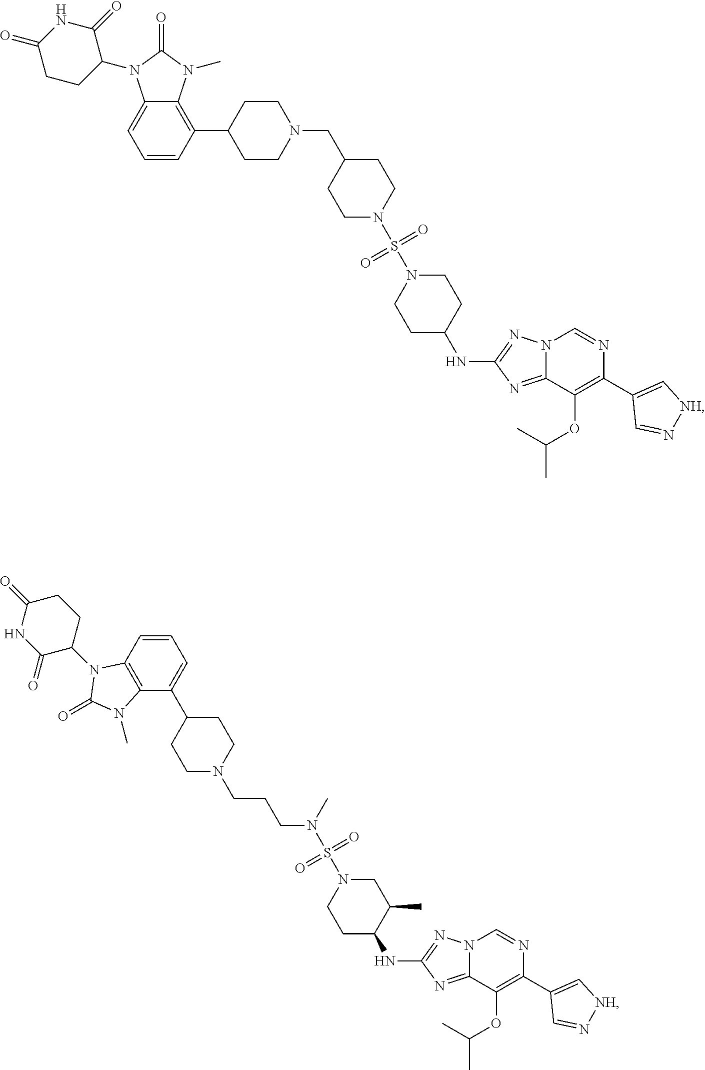 [SMILES: CC(C)Oc1c(-c2cn[nH]c2)ncn2nc(NC3CCN(S(=O)(=O)N4CCC(CN5CCC(c6cccc7c6n(C)c(=O)n7C6CCC(=O)NC6=O)CC5)CC4)CC3)nc12 ; CC(C)Oc1c(-c2cn[nH]c2)ncn2nc(N[C@H]3CCN(S(=O)(=O)N(C)CCCN4CCC(c5cccc6c5n(C)c(=O)n6C5CCC(=O)NC5=O)CC4)C[C@H]3C)nc12]

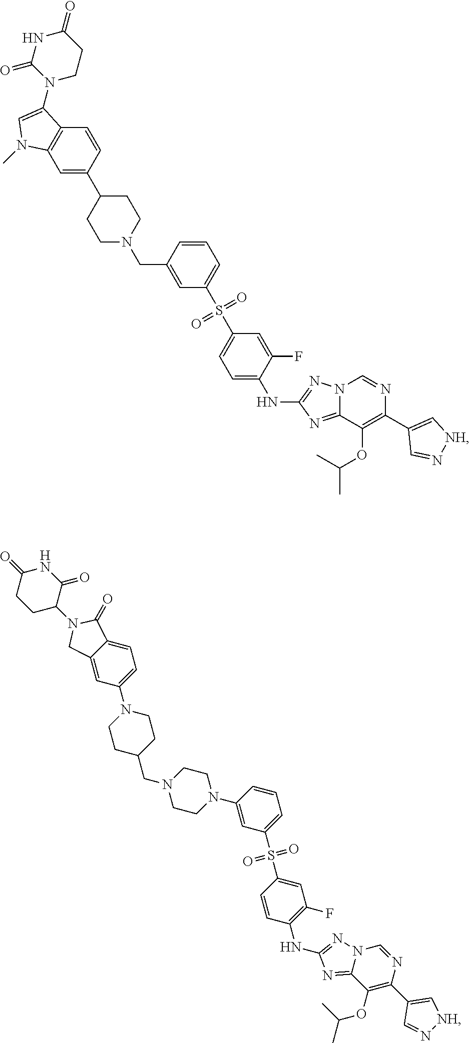 [SMILES: CC(C)Oc1c(-c2cn[nH]c2)ncn2nc(Nc3ccc(S(=O)(=O)c4cccc(CN5CCC(c6ccc7c(N8CCC(=O)NC8=O)cn(C)c7c6)CC5)c4)cc3F)nc12 ; CC(C)Oc1c(-c2cn[nH]c2)ncn2nc(Nc3ccc(S(=O)(=O)c4cccc(N5CCN(CC6CCN(c7ccc8c(c7)CN(C7CCC(=O)NC7=O)C8=O)CC6)CC5)c4)cc3F)nc12]

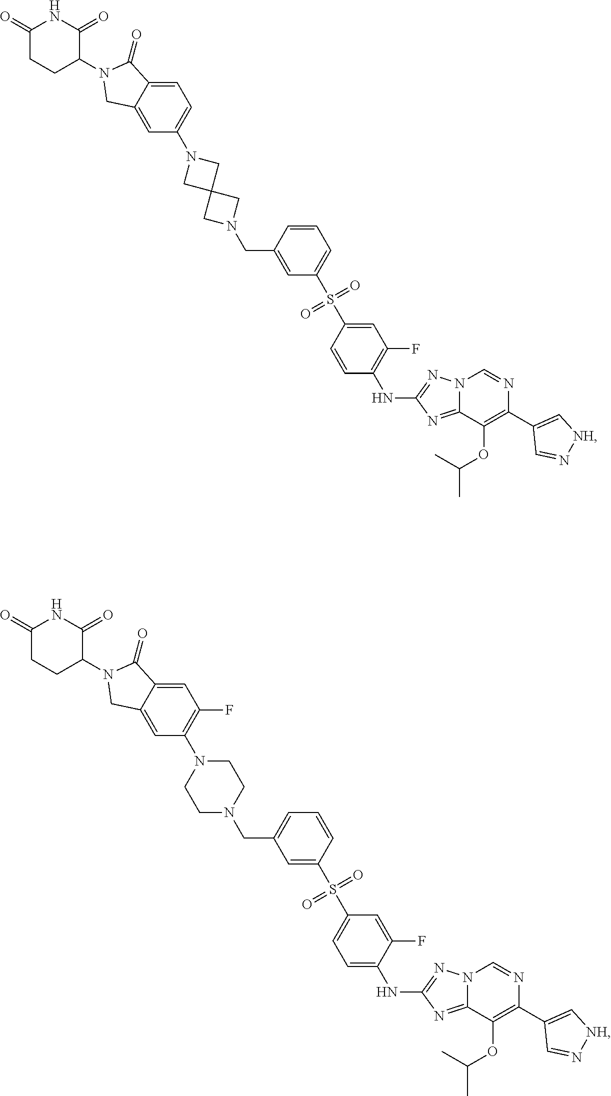 [SMILES: CC(C)Oc1c(-c2cn[nH]c2)ncn2nc(Nc3ccc(S(=O)(=O)c4cccc(CN5CC6(C5)CN(c5ccc7c(c5)CN(C5CCC(=O)NC5=O)C7=O)C6)c4)cc3F)nc12 ; CC(C)Oc1c(-c2cn[nH]c2)ncn2nc(Nc3ccc(S(=O)(=O)c4cccc(CN5CCN(c6cc7c(cc6F)C(=O)N(C6CCC(=O)NC6=O)C7)CC5)c4)cc3F)nc12]

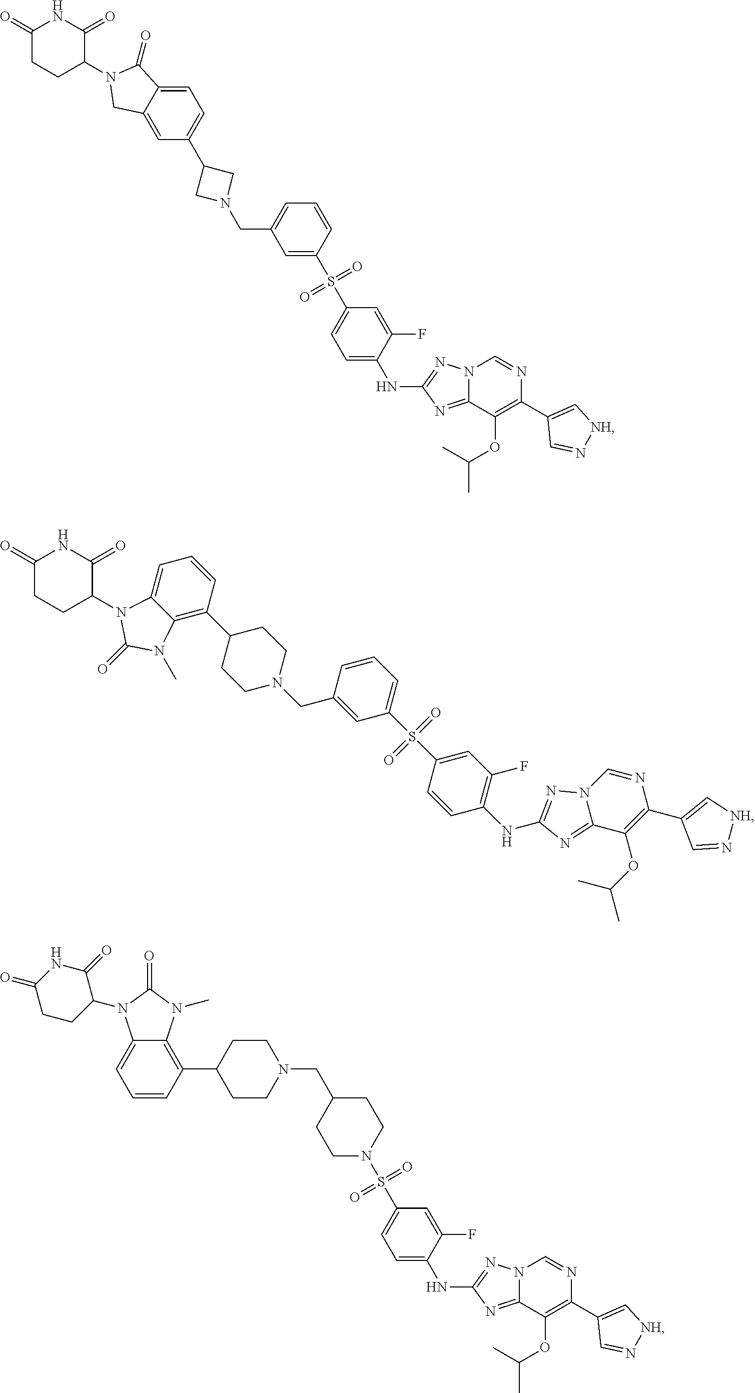 [SMILES: CC(C)Oc1c(-c2cn[nH]c2)ncn2nc(Nc3ccc(S(=O)(=O)c4cccc(CN5CC(c6ccc7c(c6)CN(C6CCC(=O)NC6=O)C7=O)C5)c4)cc3F)nc12 ; CC(C)Oc1c(-c2cn[nH]c2)ncn2nc(Nc3ccc(S(=O)(=O)c4cccc(CN5CCC(c6cccc7c6n(C)c(=O)n7C6CCC(=O)NC6=O)CC5)c4)cc3F)nc12 ; CC(C)Oc1c(-c2cn[nH]c2)ncn2nc(Nc3ccc(S(=O)(=O)N4CCC(CN5CCC(c6cccc7c6n(C)c(=O)n7C6CCC(=O)NC6=O)CC5)CC4)cc3F)nc12]

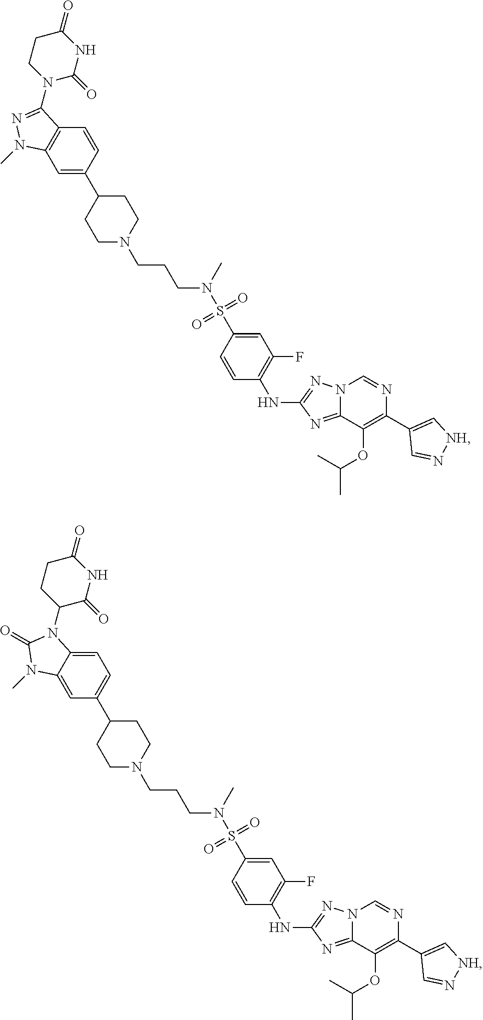 [SMILES: CC(C)Oc1c(-c2cn[nH]c2)ncn2nc(Nc3ccc(S(=O)(=O)N(C)CCCN4CCC(c5ccc6c(N7CCC(=O)NC7=O)nn(C)c6c5)CC4)cc3F)nc12 ; CC(C)Oc1c(-c2cn[nH]c2)ncn2nc(Nc3ccc(S(=O)(=O)N(C)CCCN4CCC(c5ccc6c(c5)n(C)c(=O)n6C5CCC(=O)NC5=O)CC4)cc3F)nc12]

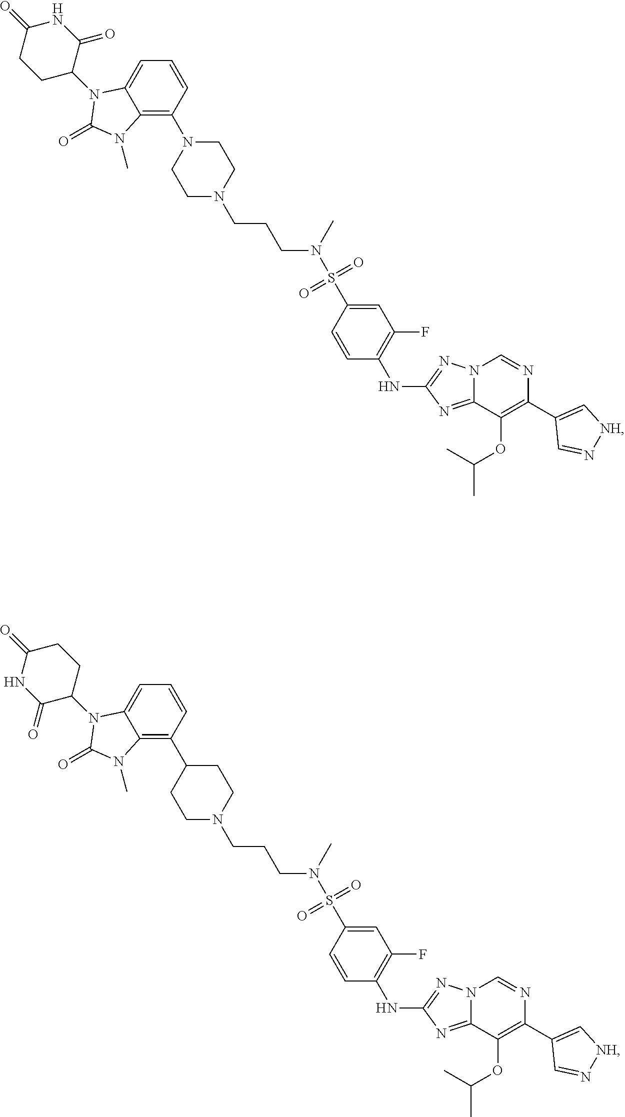 [SMILES: CC(C)Oc1c(-c2cn[nH]c2)ncn2nc(Nc3ccc(S(=O)(=O)N(C)CCCN4CCN(c5cccc6c5n(C)c(=O)n6C5CCC(=O)NC5=O)CC4)cc3F)nc12 ; CC(C)Oc1c(-c2cn[nH]c2)ncn2nc(Nc3ccc(S(=O)(=O)N(C)CCCN4CCC(c5cccc6c5n(C)c(=O)n6C5CCC(=O)NC5=O)CC4)cc3F)nc12]

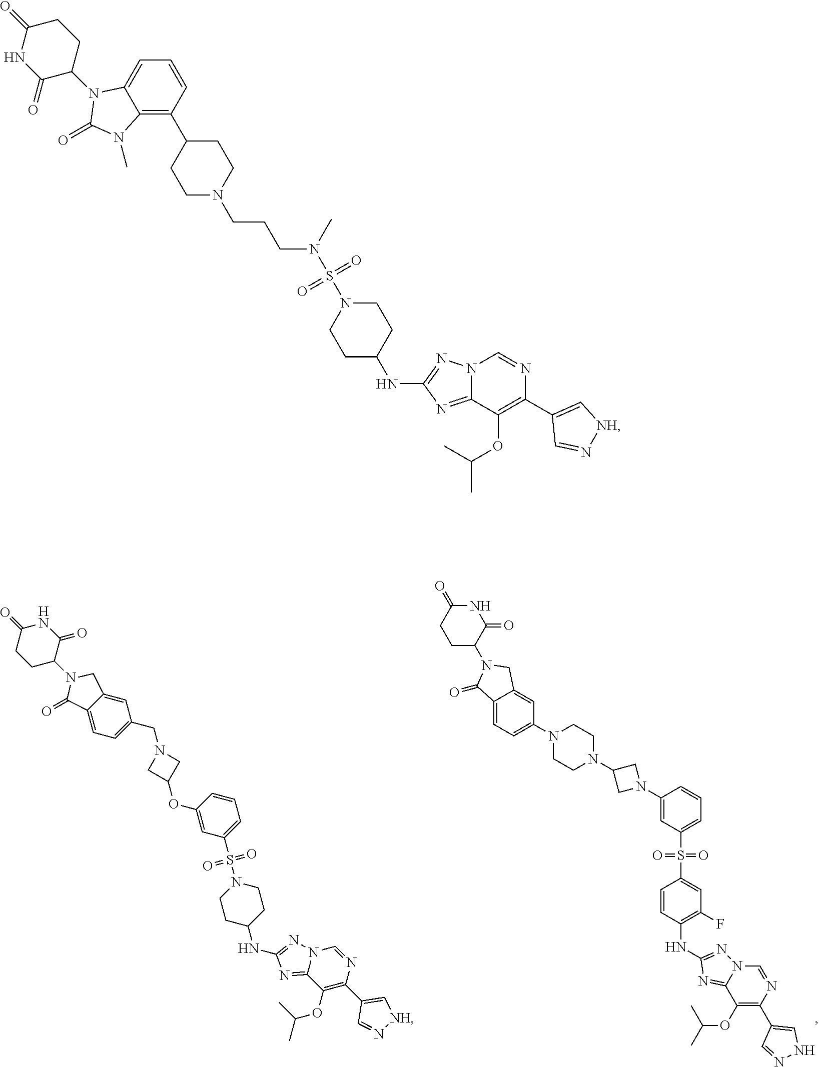 [SMILES: CC(C)Oc1c(-c2cn[nH]c2)ncn2nc(NC3CCN(S(=O)(=O)N(C)CCCN4CCC(c5cccc6c5n(C)c(=O)n6C5CCC(=O)NC5=O)CC4)CC3)nc12 ; CC(C)Oc1c(-c2cn[nH]c2)ncn2nc(NC3CCN(S(=O)(=O)c4cccc(OC5CN(Cc6ccc7c(c6)CN(C6CCC(=O)NC6=O)C7=O)C5)c4)CC3)nc12 ; CC(C)Oc1c(-c2cn[nH]c2)ncn2nc(Nc3ccc(S(=O)(=O)c4cccc(N5CC(N6CCN(c7ccc8c(c7)CN(C7CCC(=O)NC7=O)C8=O)CC6)C5)c4)cc3F)nc12]

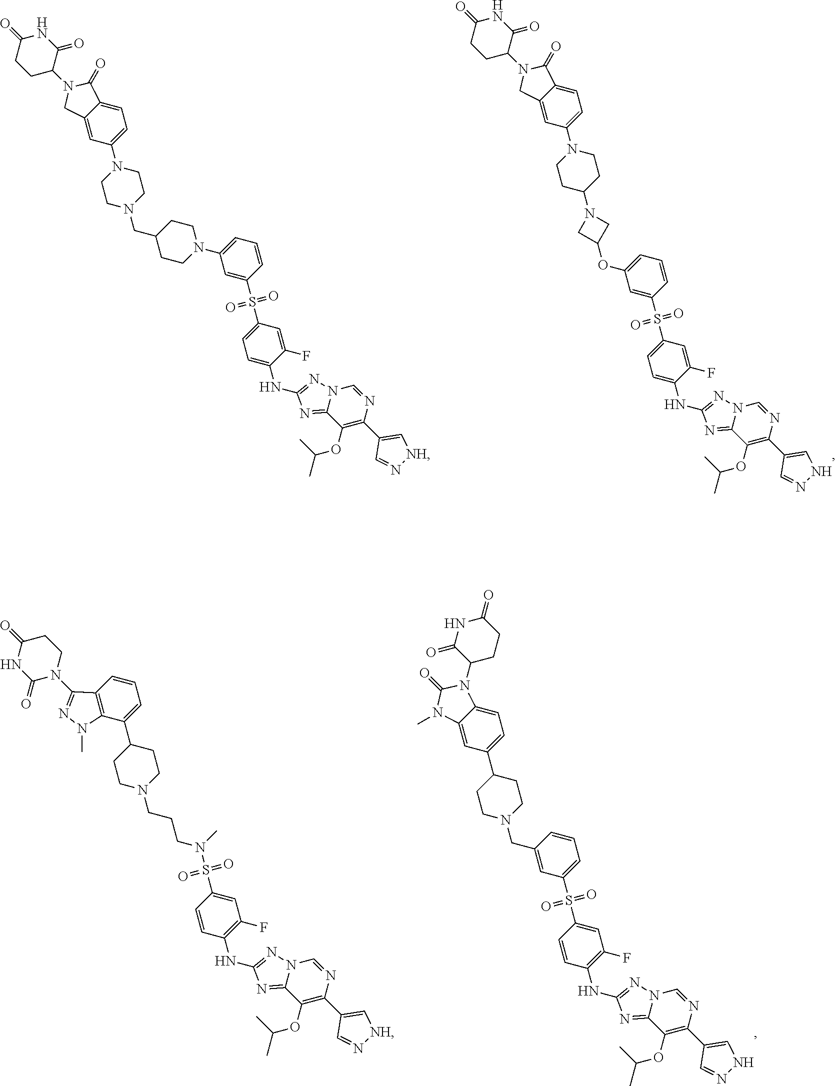 [SMILES: CC(C)Oc1c(-c2cn[nH]c2)ncn2nc(Nc3ccc(S(=O)(=O)c4cccc(N5CCC(CN6CCN(c7ccc8c(c7)CN(C7CCC(=O)NC7=O)C8=O)CC6)CC5)c4)cc3F)nc12 ; CC(C)Oc1c(-c2cn[nH]c2)ncn2nc(Nc3ccc(S(=O)(=O)c4cccc(OC5CN(C6CCN(c7ccc8c(c7)CN(C7CCC(=O)NC7=O)C8=O)CC6)C5)c4)cc3F)nc12 ; CC(C)Oc1c(-c2cn[nH]c2)ncn2nc(Nc3ccc(S(=O)(=O)N(C)CCCN4CCC(c5cccc6c(N7CCC(=O)NC7=O)nn(C)c56)CC4)cc3F)nc12 ; CC(C)Oc1c(-c2cn[nH]c2)ncn2nc(Nc3ccc(S(=O)(=O)c4cccc(CN5CCC(c6ccc7c(c6)n(C)c(=O)n7C6CCC(=O)NC6=O)CC5)c4)cc3F)nc12]

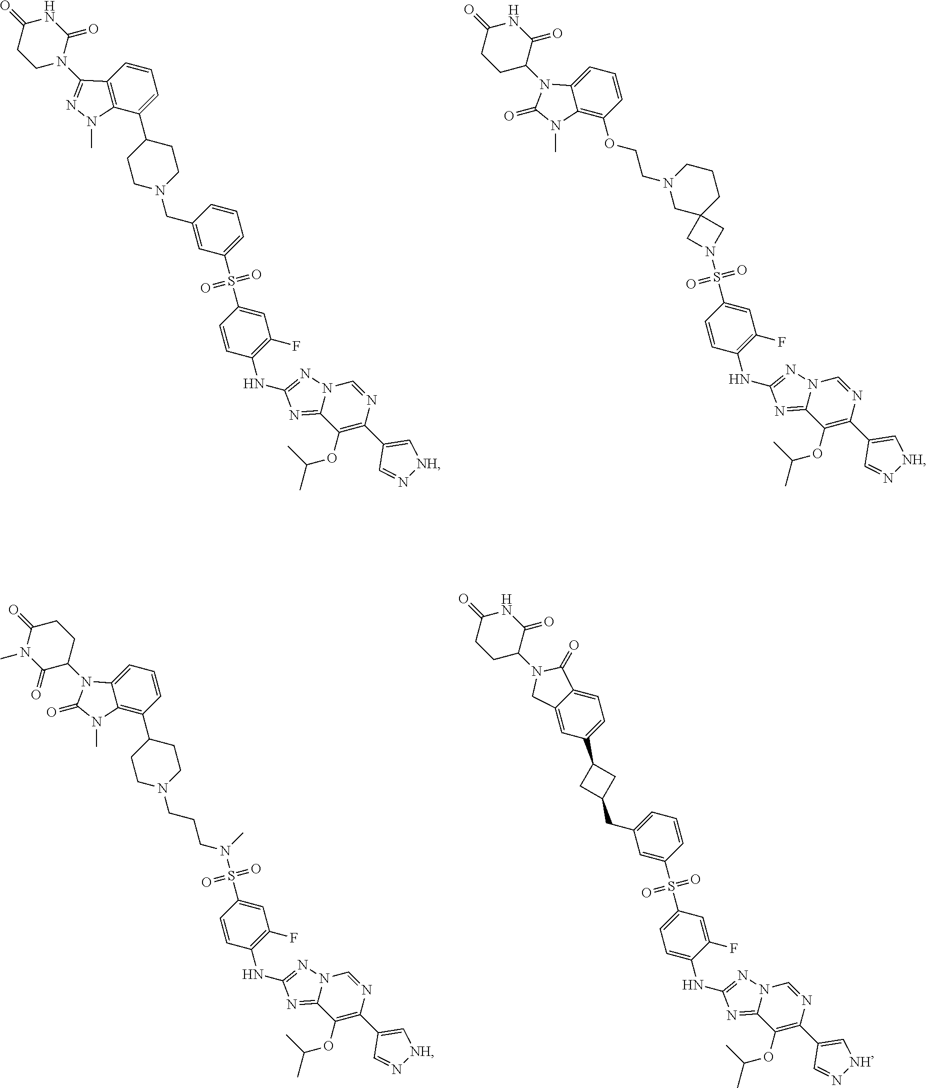 [SMILES: CC(C)Oc1c(-c2cn[nH]c2)ncn2nc(Nc3ccc(S(=O)(=O)c4cccc(CN5CCC(c6cccc7c(N8CCC(=O)NC8=O)nn(C)c67)CC5)c4)cc3F)nc12 ; CC(C)Oc1c(-c2cn[nH]c2)ncn2nc(Nc3ccc(S(=O)(=O)N4CC5(CCCN(CCOc6cccc7c6n(C)c(=O)n7C6CCC(=O)NC6=O)C5)C4)cc3F)nc12 ; CC(C)Oc1c(-c2cn[nH]c2)ncn2nc(Nc3ccc(S(=O)(=O)N(C)CCCN4CCC(c5cccc6c5n(C)c(=O)n6C5CCC(=O)N(C)C5=O)CC4)cc3F)nc12 ; CC(C)Oc1c(-c2cn[nH]c2)ncn2nc(Nc3ccc(S(=O)(=O)c4cccc(C[C@H]5C[C@@H](c6ccc7c(c6)CN(C6CCC(=O)NC6=O)C7=O)C5)c4)cc3F)nc12]

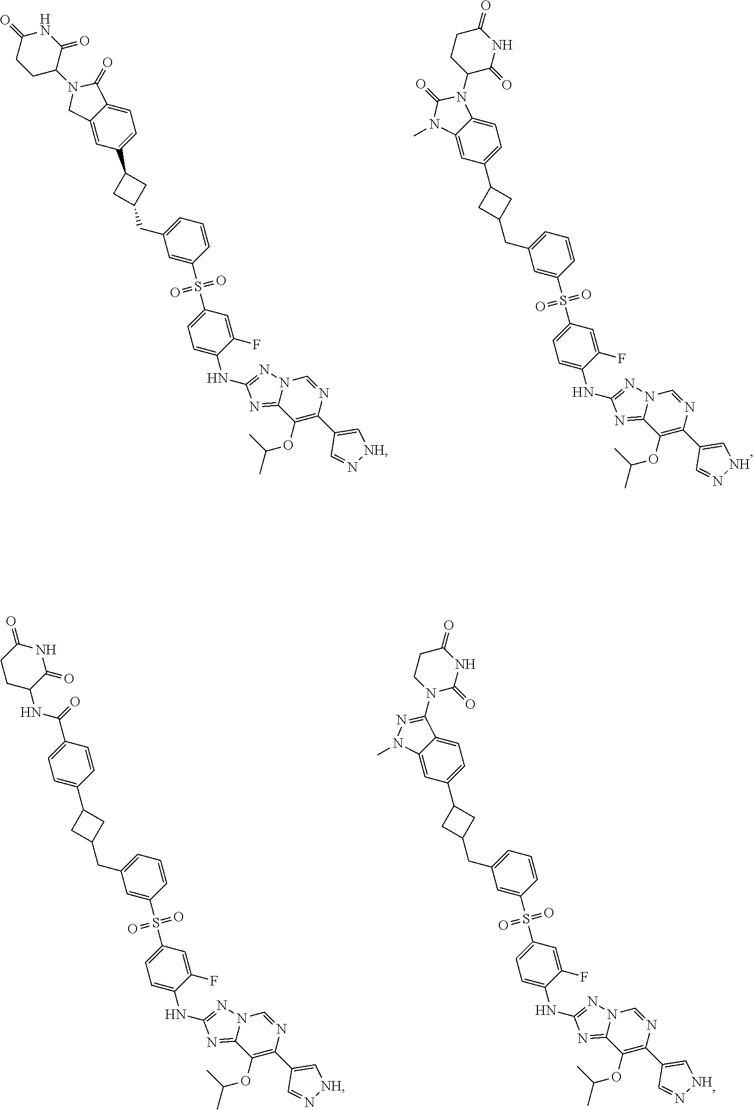 [SMILES: CC(C)Oc1c(-c2cn[nH]c2)ncn2nc(Nc3ccc(S(=O)(=O)c4cccc(C[C@H]5C[C@H](c6ccc7c(c6)CN(C6CCC(=O)NC6=O)C7=O)C5)c4)cc3F)nc12 ; CC(C)Oc1c(-c2cn[nH]c2)ncn2nc(Nc3ccc(S(=O)(=O)c4cccc(CC5CC(c6ccc7c(c6)n(C)c(=O)n7C6CCC(=O)NC6=O)C5)c4)cc3F)nc12 ; CC(C)Oc1c(-c2cn[nH]c2)ncn2nc(Nc3ccc(S(=O)(=O)c4cccc(CC5CC(c6ccc(C(=O)NC7CCC(=O)NC7=O)cc6)C5)c4)cc3F)nc12 ; CC(C)Oc1c(-c2cn[nH]c2)ncn2nc(Nc3ccc(S(=O)(=O)c4cccc(CC5CC(c6ccc7c(N8CCC(=O)NC8=O)nn(C)c7c6)C5)c4)cc3F)nc12]

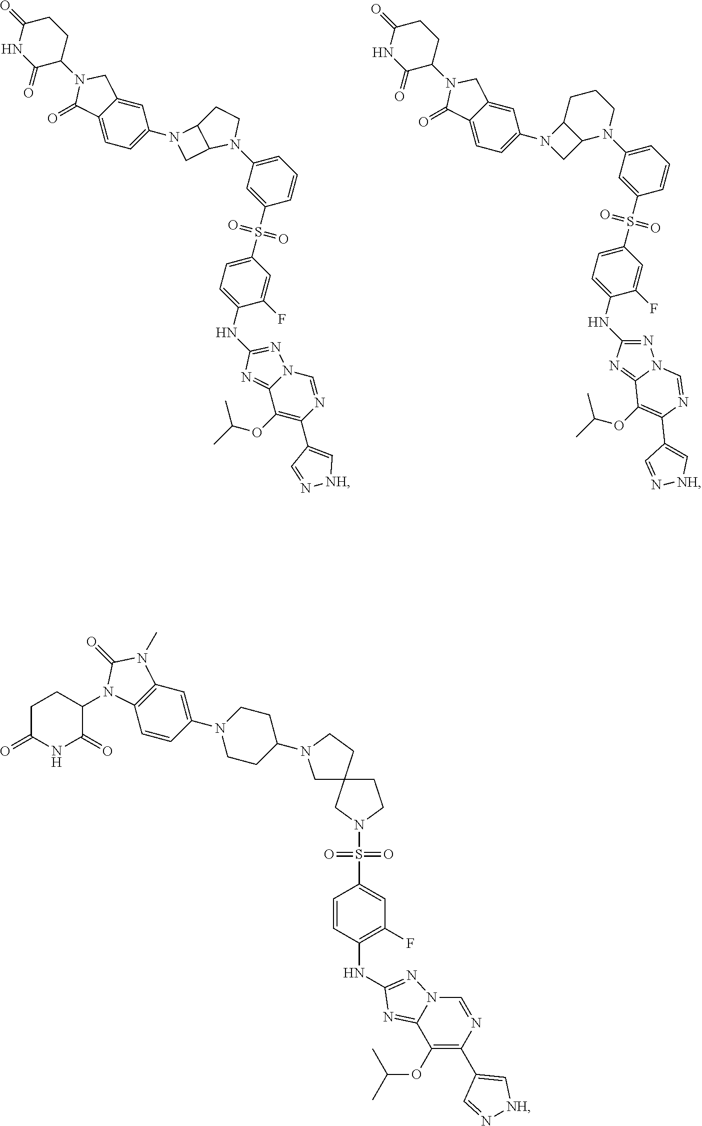 [SMILES: CC(C)Oc1c(-c2cn[nH]c2)ncn2nc(Nc3ccc(S(=O)(=O)c4cccc(N5CCC6C5CN6c5ccc6c(c5)CN(C5CCC(=O)NC5=O)C6=O)c4)cc3F)nc12 ; CC(C)Oc1c(-c2cn[nH]c2)ncn2nc(Nc3ccc(S(=O)(=O)c4cccc(N5CCCC6C5CN6c5ccc6c(c5)CN(C5CCC(=O)NC5=O)C6=O)c4)cc3F)nc12 ; CC(C)Oc1c(-c2cn[nH]c2)ncn2nc(Nc3ccc(S(=O)(=O)N4CCC5(CCN(C6CCN(c7ccc8c(c7)n(C)c(=O)n8C7CCC(=O)NC7=O)CC6)C5)C4)cc3F)nc12]

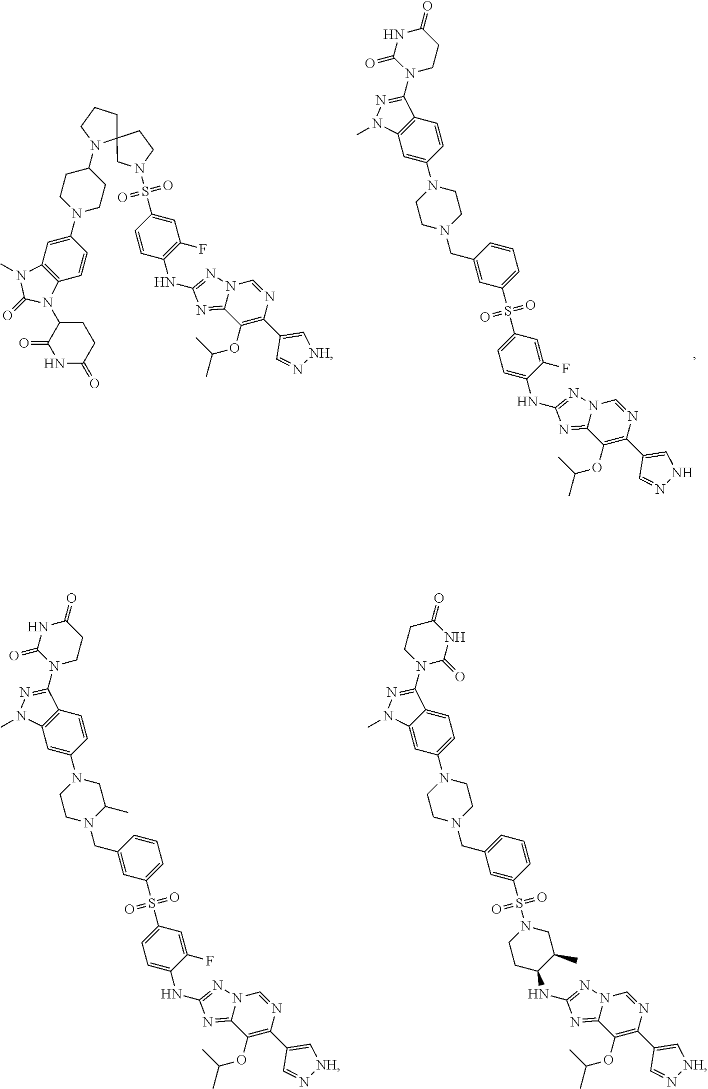 [SMILES: CC(C)Oc1c(-c2cn[nH]c2)ncn2nc(Nc3ccc(S(=O)(=O)N4CCC5(CCCN5C5CCN(c6ccc7c(c6)n(C)c(=O)n7C6CCC(=O)NC6=O)CC5)C4)cc3F)nc12 ; CC(C)Oc1c(-c2cn[nH]c2)ncn2nc(Nc3ccc(S(=O)(=O)c4cccc(CN5CCN(c6ccc7c(N8CCC(=O)NC8=O)nn(C)c7c6)CC5)c4)cc3F)nc12 ; CC(C)Oc1c(-c2cn[nH]c2)ncn2nc(Nc3ccc(S(=O)(=O)c4cccc(CN5CCN(c6ccc7c(N8CCC(=O)NC8=O)nn(C)c7c6)CC5C)c4)cc3F)nc12 ; CC(C)Oc1c(-c2cn[nH]c2)ncn2nc(N[C@H]3CCN(S(=O)(=O)c4cccc(CN5CCN(c6ccc7c(N8CCC(=O)NC8=O)nn(C)c7c6)CC5)c4)C[C@H]3C)nc12]

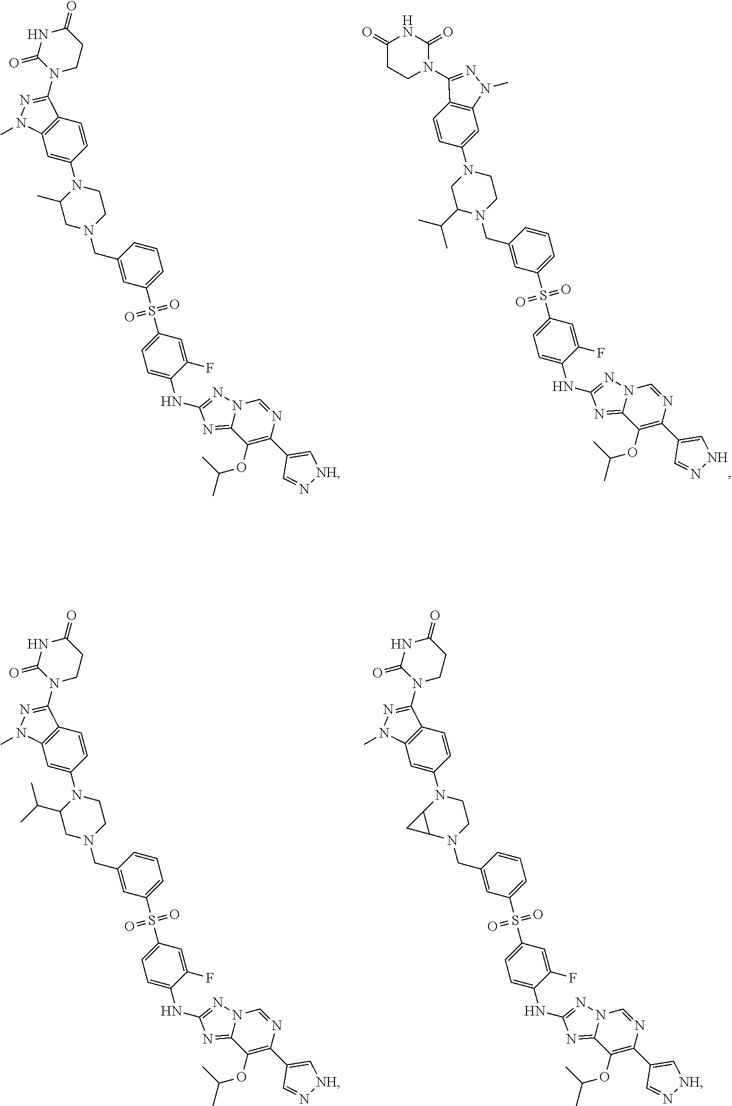 [SMILES: CC(C)Oc1c(-c2cn[nH]c2)ncn2nc(Nc3ccc(S(=O)(=O)c4cccc(CN5CCN(c6ccc7c(N8CCC(=O)NC8=O)nn(C)c7c6)C(C)C5)c4)cc3F)nc12 ; CC(C)Oc1c(-c2cn[nH]c2)ncn2nc(Nc3ccc(S(=O)(=O)c4cccc(CN5CCN(c6ccc7c(N8CCC(=O)NC8=O)nn(C)c7c6)CC5C(C)C)c4)cc3F)nc12 ; CC(C)Oc1c(-c2cn[nH]c2)ncn2nc(Nc3ccc(S(=O)(=O)c4cccc(CN5CCN(c6ccc7c(N8CCC(=O)NC8=O)nn(C)c7c6)C(C(C)C)C5)c4)cc3F)nc12 ; CC(C)Oc1c(-c2cn[nH]c2)ncn2nc(Nc3ccc(S(=O)(=O)c4cccc(CN5CCN(c6ccc7c(N8CCC(=O)NC8=O)nn(C)c7c6)C6CC65)c4)cc3F)nc12]

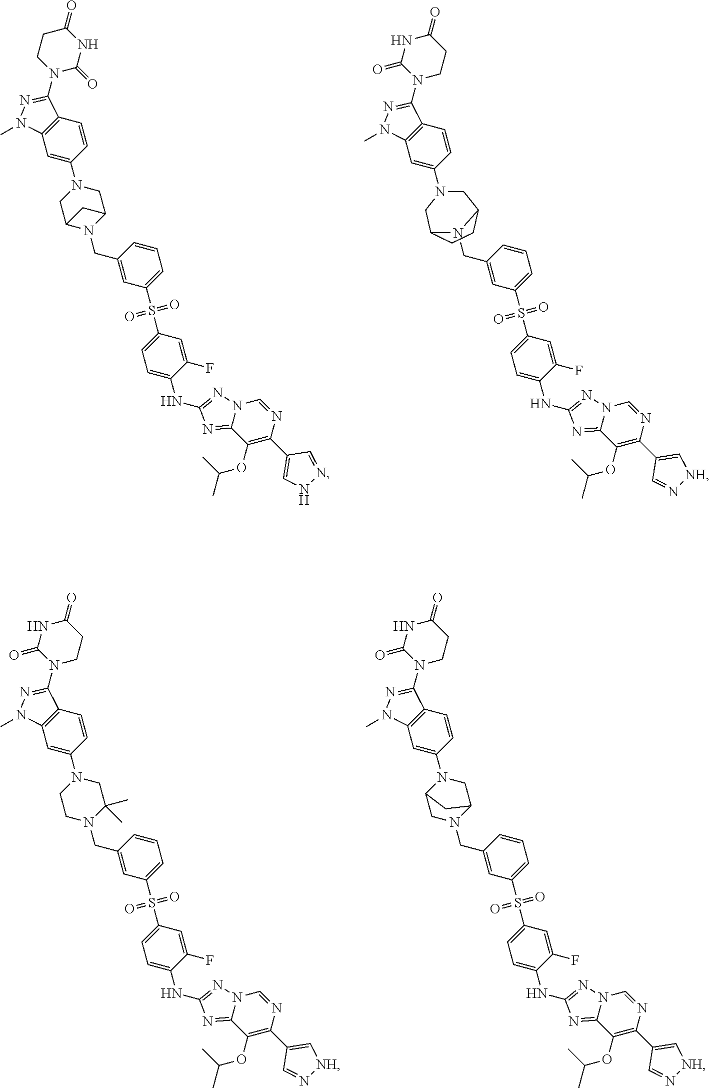 [SMILES: CC(C)Oc1c(-c2cn[nH]c2)ncn2nc(Nc3ccc(S(=O)(=O)c4cccc(CN5C6CC5CN(c5ccc7c(N8CCC(=O)NC8=O)nn(C)c7c5)C6)c4)cc3F)nc12 ; CC(C)Oc1c(-c2cn[nH]c2)ncn2nc(Nc3ccc(S(=O)(=O)c4cccc(CN5C6CCC5CN(c5ccc7c(N8CCC(=O)NC8=O)nn(C)c7c5)C6)c4)cc3F)nc12 ; CC(C)Oc1c(-c2cn[nH]c2)ncn2nc(Nc3ccc(S(=O)(=O)c4cccc(CN5CCN(c6ccc7c(N8CCC(=O)NC8=O)nn(C)c7c6)CC5(C)C)c4)cc3F)nc12 ; CC(C)Oc1c(-c2cn[nH]c2)ncn2nc(Nc3ccc(S(=O)(=O)c4cccc(CN5CC6CC5CN6c5ccc6c(N7CCC(=O)NC7=O)nn(C)c6c5)c4)cc3F)nc12]

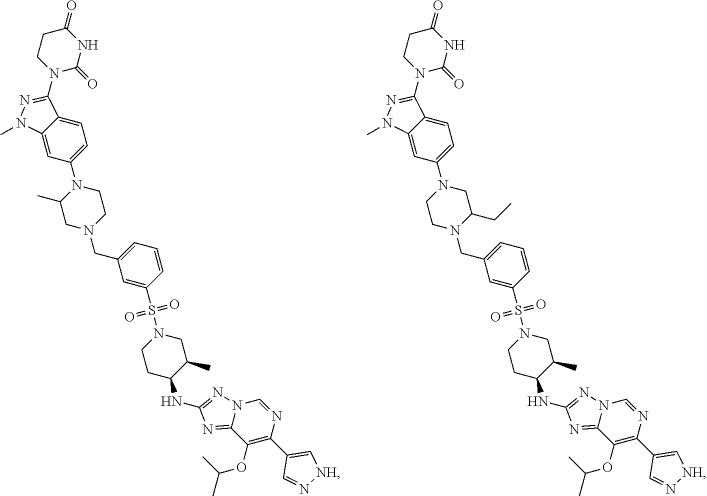 [SMILES: CC(C)Oc1c(-c2cn[nH]c2)ncn2nc(N[C@H]3CCN(S(=O)(=O)c4cccc(CN5CCN(c6ccc7c(N8CCC(=O)NC8=O)nn(C)c7c6)C(C)C5)c4)C[C@H]3C)nc12 ; CCC1CN(c2ccc3c(N4CCC(=O)NC4=O)nn(C)c3c2)CCN1Cc1cccc(S(=O)(=O)N2CC[C@H](Nc3nc4c(OC(C)C)c(-c5cn[nH]c5)ncn4n3)[C@H](C)C2)c1]

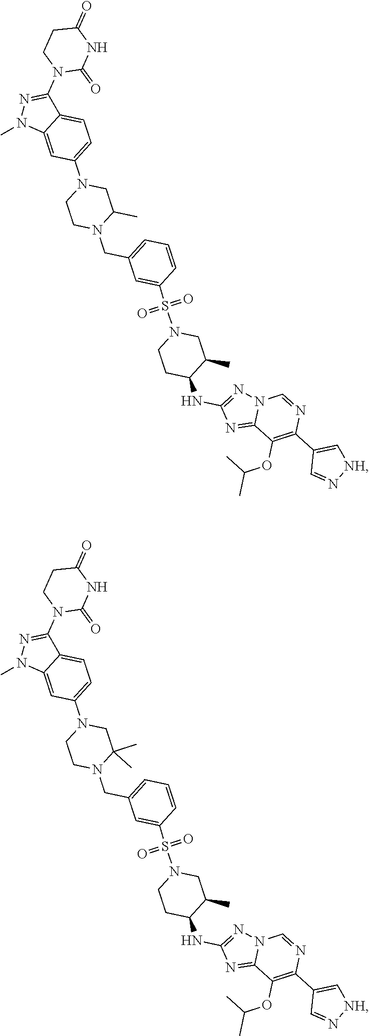 [SMILES: CC(C)Oc1c(-c2cn[nH]c2)ncn2nc(N[C@H]3CCN(S(=O)(=O)c4cccc(CN5CCN(c6ccc7c(N8CCC(=O)NC8=O)nn(C)c7c6)CC5C)c4)C[C@H]3C)nc12 ; CC(C)Oc1c(-c2cn[nH]c2)ncn2nc(N[C@H]3CCN(S(=O)(=O)c4cccc(CN5CCN(c6ccc7c(N8CCC(=O)NC8=O)nn(C)c7c6)CC5(C)C)c4)C[C@H]3C)nc12]

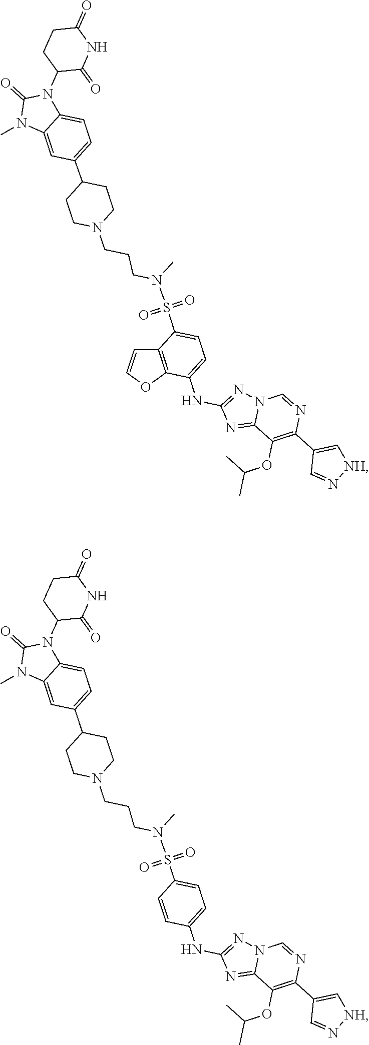 [SMILES: CC(C)Oc1c(-c2cn[nH]c2)ncn2nc(Nc3ccc(S(=O)(=O)N(C)CCCN4CCC(c5ccc6c(c5)n(C)c(=O)n6C5CCC(=O)NC5=O)CC4)c4ccoc34)nc12 ; CC(C)Oc1c(-c2cn[nH]c2)ncn2nc(Nc3ccc(S(=O)(=O)N(C)CCCN4CCC(c5ccc6c(c5)n(C)c(=O)n6C5CCC(=O)NC5=O)CC4)cc3)nc12]

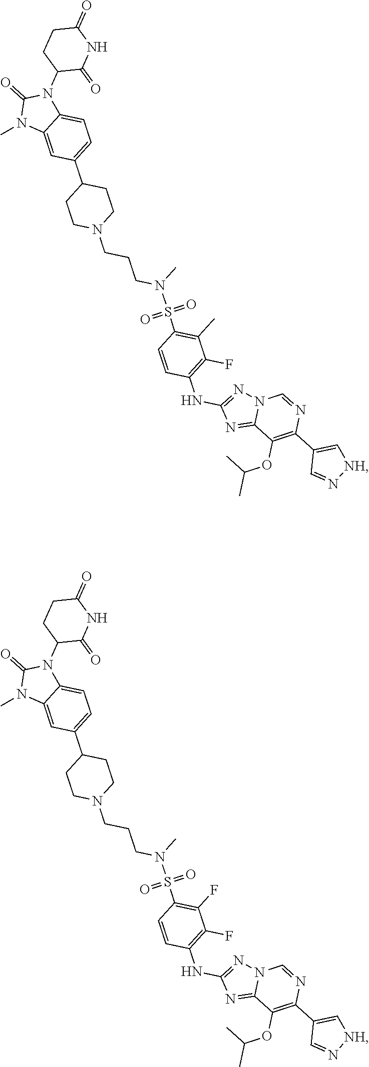 [SMILES: Cc1c(S(=O)(=O)N(C)CCCN2CCC(c3ccc4c(c3)n(C)c(=O)n4C3CCC(=O)NC3=O)CC2)ccc(Nc2nc3c(OC(C)C)c(-c4cn[nH]c4)ncn3n2)c1F ; CC(C)Oc1c(-c2cn[nH]c2)ncn2nc(Nc3ccc(S(=O)(=O)N(C)CCCN4CCC(c5ccc6c(c5)n(C)c(=O)n6C5CCC(=O)NC5=O)CC4)c(F)c3F)nc12]

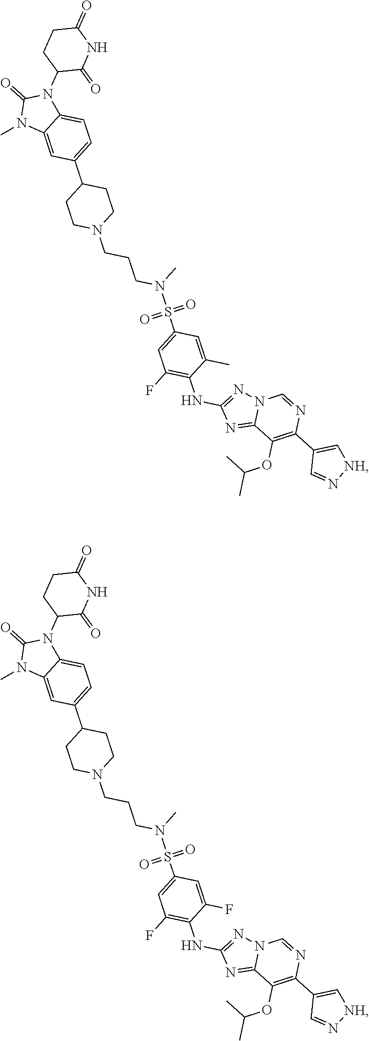 [SMILES: Cc1cc(S(=O)(=O)N(C)CCCN2CCC(c3ccc4c(c3)n(C)c(=O)n4C3CCC(=O)NC3=O)CC2)cc(F)c1Nc1nc2c(OC(C)C)c(-c3cn[nH]c3)ncn2n1 ; CC(C)Oc1c(-c2cn[nH]c2)ncn2nc(Nc3c(F)cc(S(=O)(=O)N(C)CCCN4CCC(c5ccc6c(c5)n(C)c(=O)n6C5CCC(=O)NC5=O)CC4)cc3F)nc12]

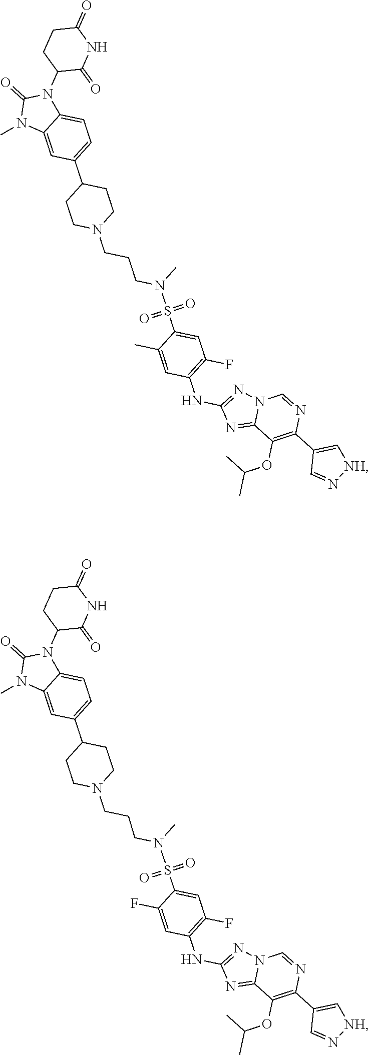 [SMILES: Cc1cc(Nc2nc3c(OC(C)C)c(-c4cn[nH]c4)ncn3n2)c(F)cc1S(=O)(=O)N(C)CCCN1CCC(c2ccc3c(c2)n(C)c(=O)n3C2CCC(=O)NC2=O)CC1 ; CC(C)Oc1c(-c2cn[nH]c2)ncn2nc(Nc3cc(F)c(S(=O)(=O)N(C)CCCN4CCC(c5ccc6c(c5)n(C)c(=O)n6C5CCC(=O)NC5=O)CC4)cc3F)nc12]

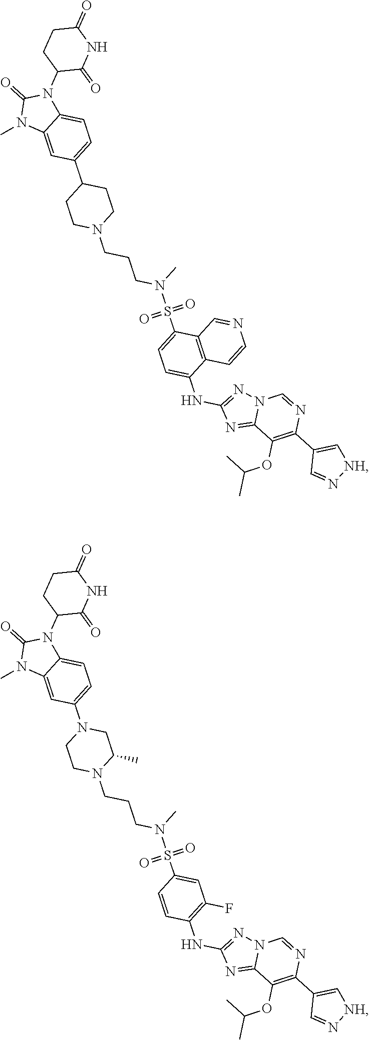 [SMILES: CC(C)Oc1c(-c2cn[nH]c2)ncn2nc(Nc3ccc(S(=O)(=O)N(C)CCCN4CCC(c5ccc6c(c5)n(C)c(=O)n6C5CCC(=O)NC5=O)CC4)c4cnccc34)nc12 ; CC(C)Oc1c(-c2cn[nH]c2)ncn2nc(Nc3ccc(S(=O)(=O)N(C)CCCN4CCN(c5ccc6c(c5)n(C)c(=O)n6C5CCC(=O)NC5=O)C[C@@H]4C)cc3F)nc12]

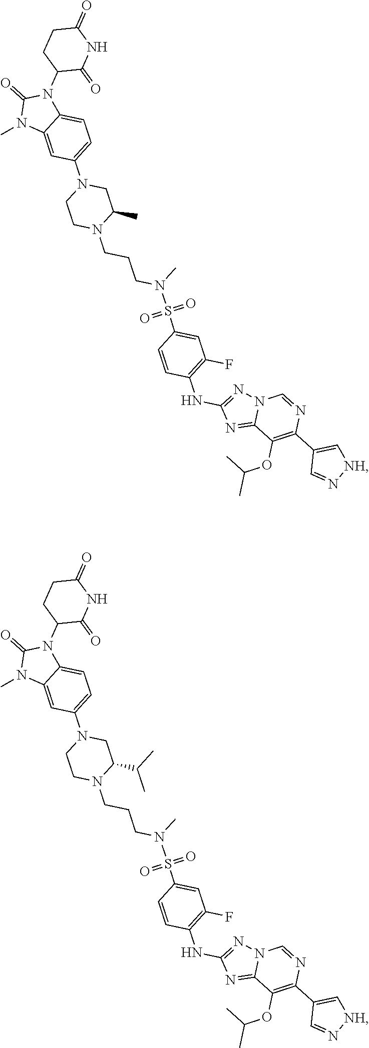 [SMILES: CC(C)Oc1c(-c2cn[nH]c2)ncn2nc(Nc3ccc(S(=O)(=O)N(C)CCCN4CCN(c5ccc6c(c5)n(C)c(=O)n6C5CCC(=O)NC5=O)C[C@H]4C)cc3F)nc12 ; CC(C)Oc1c(-c2cn[nH]c2)ncn2nc(Nc3ccc(S(=O)(=O)N(C)CCCN4CCN(c5ccc6c(c5)n(C)c(=O)n6C5CCC(=O)NC5=O)C[C@@H]4C(C)C)cc3F)nc12]

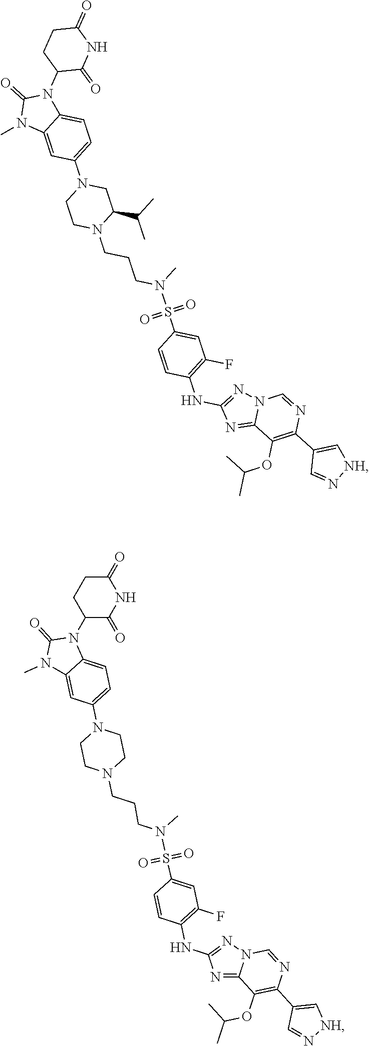 [SMILES: CC(C)Oc1c(-c2cn[nH]c2)ncn2nc(Nc3ccc(S(=O)(=O)N(C)CCCN4CCN(c5ccc6c(c5)n(C)c(=O)n6C5CCC(=O)NC5=O)C[C@H]4C(C)C)cc3F)nc12 ; CC(C)Oc1c(-c2cn[nH]c2)ncn2nc(Nc3ccc(S(=O)(=O)N(C)CCCN4CCN(c5ccc6c(c5)n(C)c(=O)n6C5CCC(=O)NC5=O)CC4)cc3F)nc12]

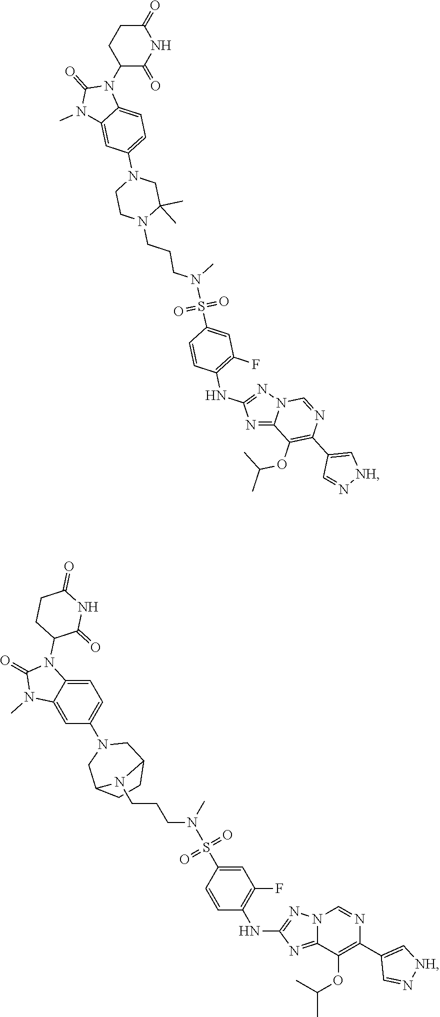 [SMILES: CC(C)Oc1c(-c2cn[nH]c2)ncn2nc(Nc3ccc(S(=O)(=O)N(C)CCCN4CCN(c5ccc6c(c5)n(C)c(=O)n6C5CCC(=O)NC5=O)CC4(C)C)cc3F)nc12 ; CC(C)Oc1c(-c2cn[nH]c2)ncn2nc(Nc3ccc(S(=O)(=O)N(C)CCCN4C5CCC4CN(c4ccc6c(c4)n(C)c(=O)n6C4CCC(=O)NC4=O)C5)cc3F)nc12]

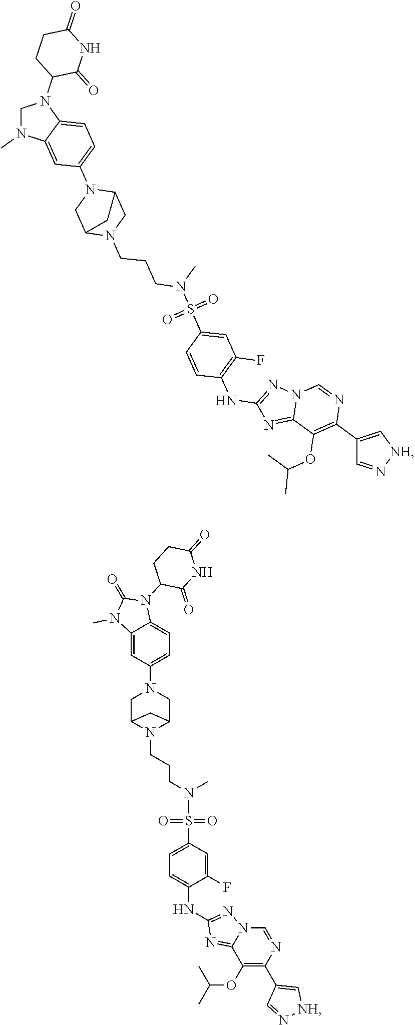 [SMILES: CC(C)Oc1c(-c2cn[nH]c2)ncn2nc(Nc3ccc(S(=O)(=O)N(C)CCCN4CC5CC4CN5c4ccc5c(c4)N(C)CN5C4CCC(=O)NC4=O)cc3F)nc12 ; CC(C)Oc1c(-c2cn[nH]c2)ncn2nc(Nc3ccc(S(=O)(=O)N(C)CCCN4C5CC4CN(c4ccc6c(c4)n(C)c(=O)n6C4CCC(=O)NC4=O)C5)cc3F)nc12]

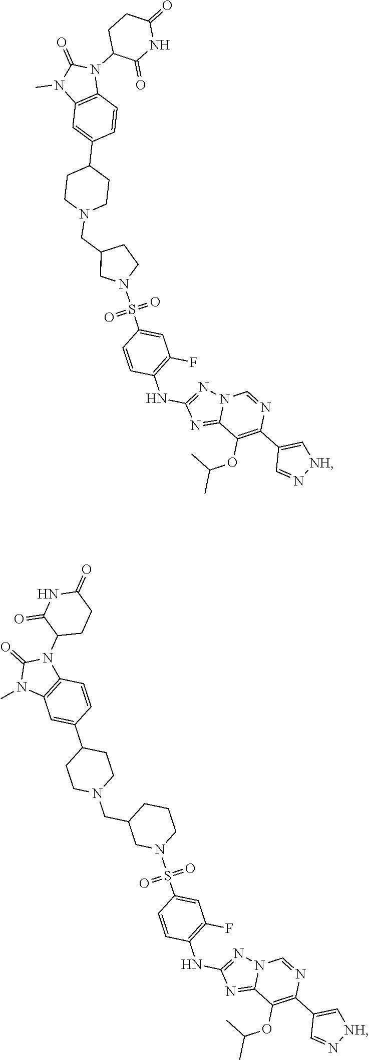 [SMILES: CC(C)Oc1c(-c2cn[nH]c2)ncn2nc(Nc3ccc(S(=O)(=O)N4CCC(CN5CCC(c6ccc7c(c6)n(C)c(=O)n7C6CCC(=O)NC6=O)CC5)C4)cc3F)nc12 ; CC(C)Oc1c(-c2cn[nH]c2)ncn2nc(Nc3ccc(S(=O)(=O)N4CCCC(CN5CCC(c6ccc7c(c6)n(C)c(=O)n7C6CCC(=O)NC6=O)CC5)C4)cc3F)nc12]

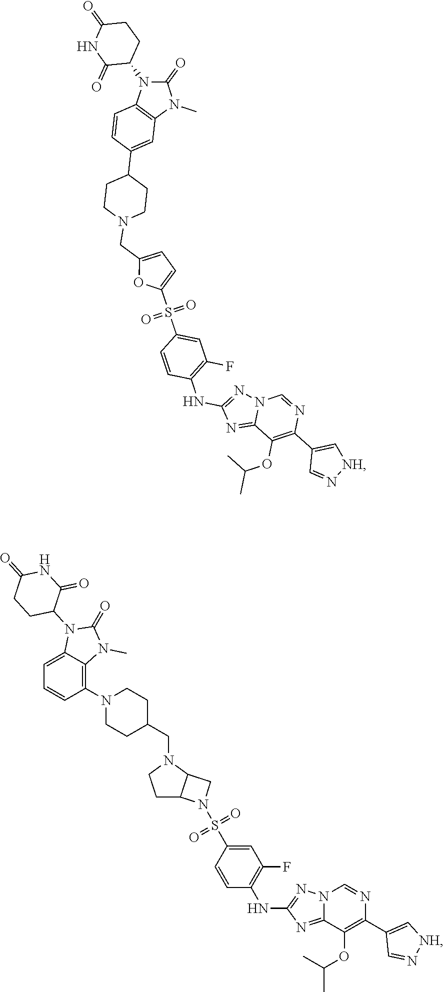 [SMILES: CC(C)Oc1c(-c2cn[nH]c2)ncn2nc(Nc3ccc(S(=O)(=O)c4ccc(CN5CCC(c6ccc7c(c6)n(C)c(=O)n7[C@H]6CCC(=O)NC6=O)CC5)o4)cc3F)nc12 ; CC(C)Oc1c(-c2cn[nH]c2)ncn2nc(Nc3ccc(S(=O)(=O)N4CC5C4CCN5CC4CCN(c5cccc6c5n(C)c(=O)n6C5CCC(=O)NC5=O)CC4)cc3F)nc12]

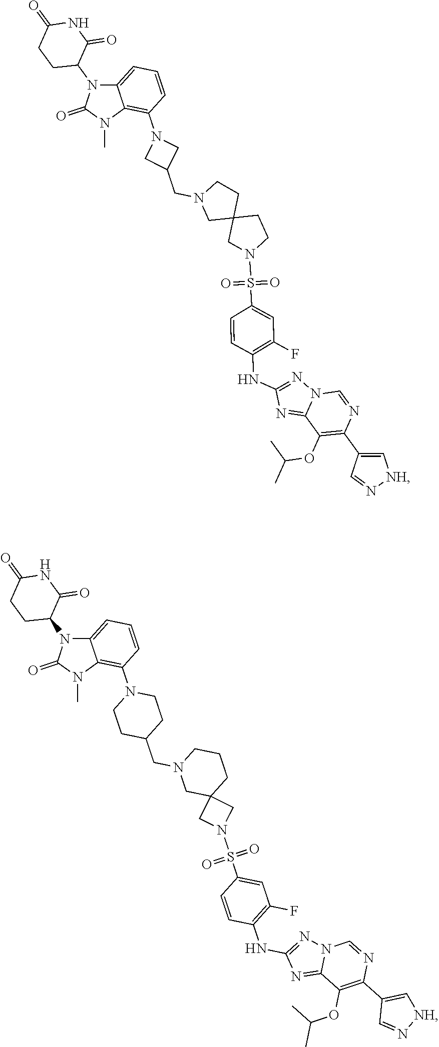 [SMILES: CC(C)Oc1c(-c2cn[nH]c2)ncn2nc(Nc3ccc(S(=O)(=O)N4CCC5(CCN(CC6CN(c7cccc8c7n(C)c(=O)n8C7CCC(=O)NC7=O)C6)C5)C4)cc3F)nc12 ; CC(C)Oc1c(-c2cn[nH]c2)ncn2nc(Nc3ccc(S(=O)(=O)N4CC5(CCCN(CC6CCN(c7cccc8c7n(C)c(=O)n8[C@H]7CCC(=O)NC7=O)CC6)C5)C4)cc3F)nc12]

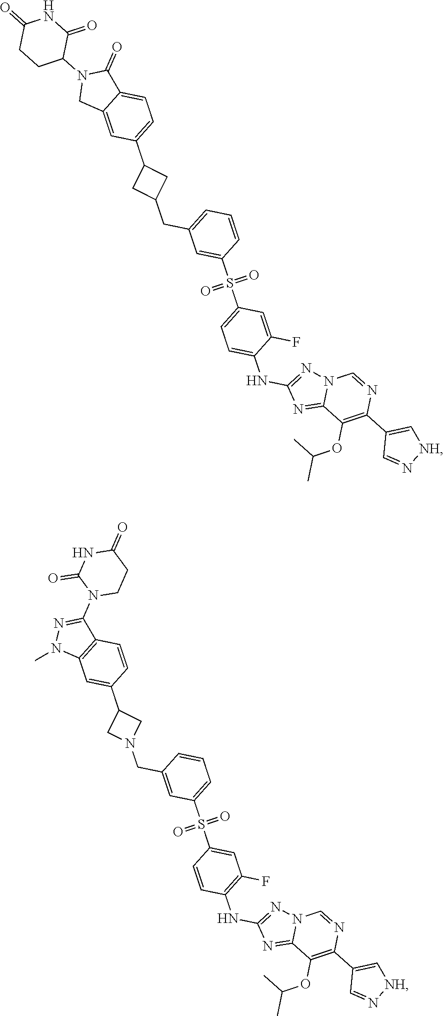 [SMILES: CC(C)Oc1c(-c2cn[nH]c2)ncn2nc(Nc3ccc(S(=O)(=O)c4cccc(CC5CC(c6ccc7c(c6)CN(C6CCC(=O)NC6=O)C7=O)C5)c4)cc3F)nc12 ; CC(C)Oc1c(-c2cn[nH]c2)ncn2nc(Nc3ccc(S(=O)(=O)c4cccc(CN5CC(c6ccc7c(N8CCC(=O)NC8=O)nn(C)c7c6)C5)c4)cc3F)nc12]

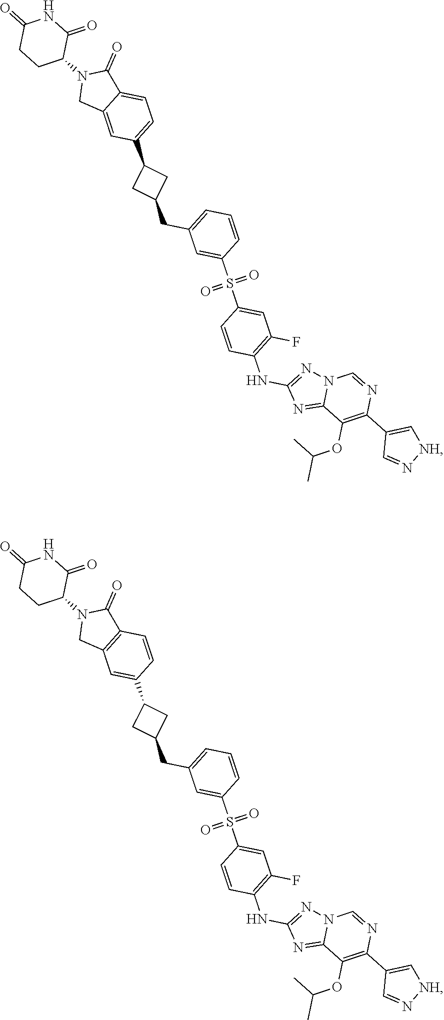 [SMILES: CC(C)Oc1c(-c2cn[nH]c2)ncn2nc(Nc3ccc(S(=O)(=O)c4cccc(C[C@H]5C[C@@H](c6ccc7c(c6)CN([C@@H]6CCC(=O)NC6=O)C7=O)C5)c4)cc3F)nc12 ; CC(C)Oc1c(-c2cn[nH]c2)ncn2nc(Nc3ccc(S(=O)(=O)c4cccc(C[C@H]5C[C@H](c6ccc7c(c6)CN([C@@H]6CCC(=O)NC6=O)C7=O)C5)c4)cc3F)nc12]

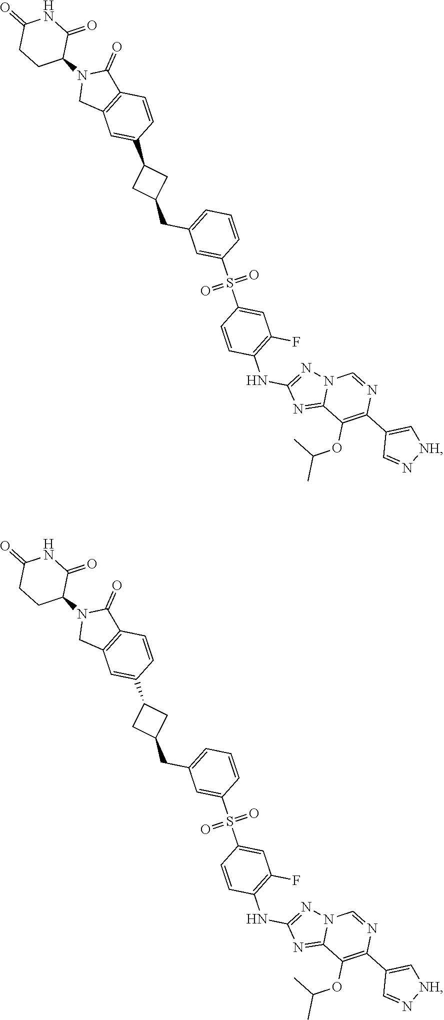 [SMILES: CC(C)Oc1c(-c2cn[nH]c2)ncn2nc(Nc3ccc(S(=O)(=O)c4cccc(C[C@H]5C[C@@H](c6ccc7c(c6)CN([C@H]6CCC(=O)NC6=O)C7=O)C5)c4)cc3F)nc12 ; CC(C)Oc1c(-c2cn[nH]c2)ncn2nc(Nc3ccc(S(=O)(=O)c4cccc(C[C@H]5C[C@H](c6ccc7c(c6)CN([C@H]6CCC(=O)NC6=O)C7=O)C5)c4)cc3F)nc12]

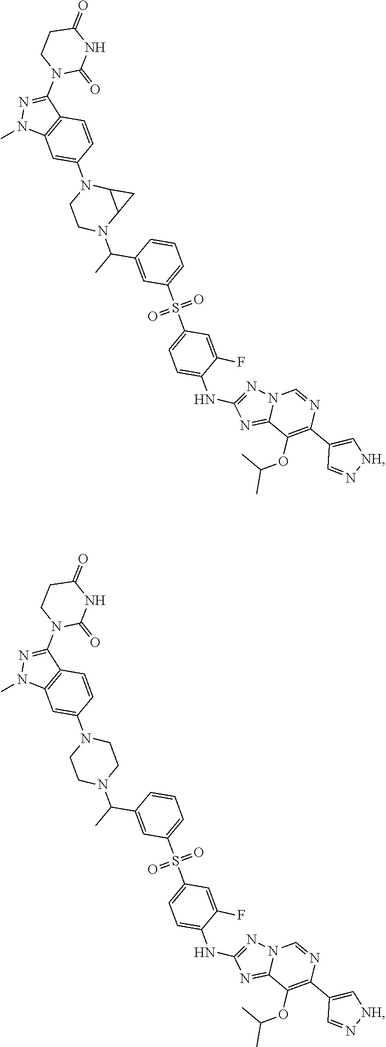 [SMILES: CC(C)Oc1c(-c2cn[nH]c2)ncn2nc(Nc3ccc(S(=O)(=O)c4cccc(C(C)N5CCN(c6ccc7c(N8CCC(=O)NC8=O)nn(C)c7c6)C6CC65)c4)cc3F)nc12 ; CC(C)Oc1c(-c2cn[nH]c2)ncn2nc(Nc3ccc(S(=O)(=O)c4cccc(C(C)N5CCN(c6ccc7c(N8CCC(=O)NC8=O)nn(C)c7c6)CC5)c4)cc3F)nc12]

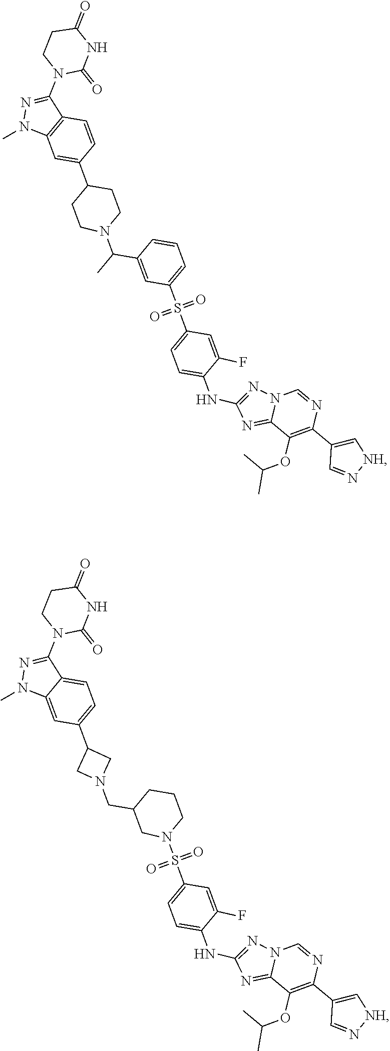 [SMILES: CC(C)Oc1c(-c2cn[nH]c2)ncn2nc(Nc3ccc(S(=O)(=O)c4cccc(C(C)N5CCC(c6ccc7c(N8CCC(=O)NC8=O)nn(C)c7c6)CC5)c4)cc3F)nc12 ; CC(C)Oc1c(-c2cn[nH]c2)ncn2nc(Nc3ccc(S(=O)(=O)N4CCCC(CN5CC(c6ccc7c(N8CCC(=O)NC8=O)nn(C)c7c6)C5)C4)cc3F)nc12]

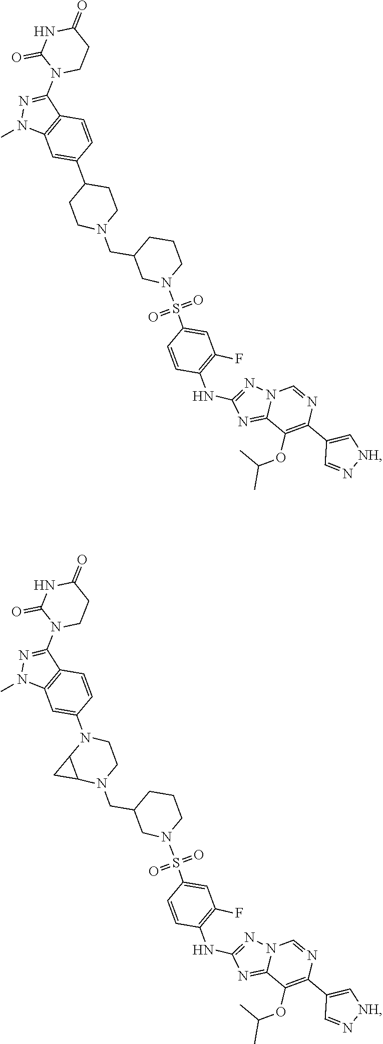 [SMILES: CC(C)Oc1c(-c2cn[nH]c2)ncn2nc(Nc3ccc(S(=O)(=O)N4CCCC(CN5CCC(c6ccc7c(N8CCC(=O)NC8=O)nn(C)c7c6)CC5)C4)cc3F)nc12 ; CC(C)Oc1c(-c2cn[nH]c2)ncn2nc(Nc3ccc(S(=O)(=O)N4CCCC(CN5CCN(c6ccc7c(N8CCC(=O)NC8=O)nn(C)c7c6)C6CC65)C4)cc3F)nc12]

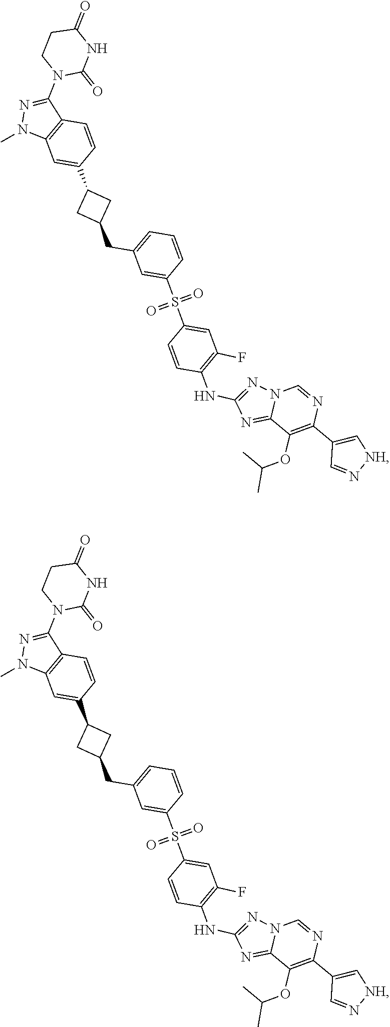 [SMILES: CC(C)Oc1c(-c2cn[nH]c2)ncn2nc(Nc3ccc(S(=O)(=O)c4cccc(C[C@H]5C[C@H](c6ccc7c(N8CCC(=O)NC8=O)nn(C)c7c6)C5)c4)cc3F)nc12 ; CC(C)Oc1c(-c2cn[nH]c2)ncn2nc(Nc3ccc(S(=O)(=O)c4cccc(C[C@H]5C[C@@H](c6ccc7c(N8CCC(=O)NC8=O)nn(C)c7c6)C5)c4)cc3F)nc12]

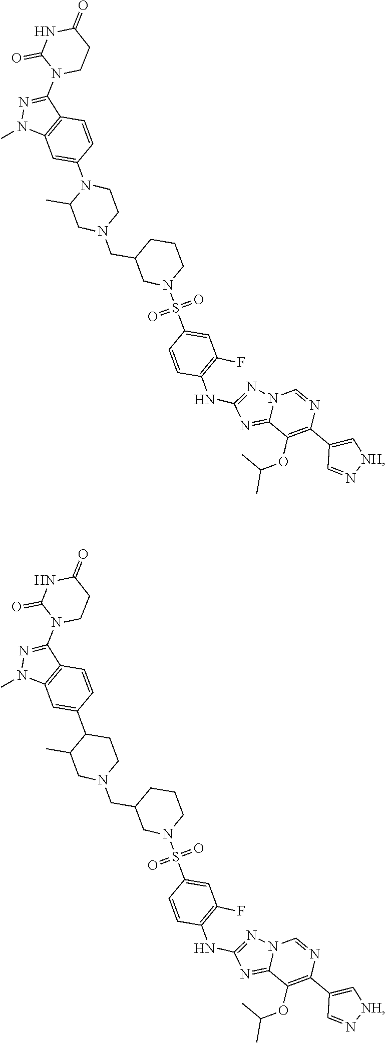 [SMILES: CC(C)Oc1c(-c2cn[nH]c2)ncn2nc(Nc3ccc(S(=O)(=O)N4CCCC(CN5CCN(c6ccc7c(N8CCC(=O)NC8=O)nn(C)c7c6)C(C)C5)C4)cc3F)nc12 ; CC(C)Oc1c(-c2cn[nH]c2)ncn2nc(Nc3ccc(S(=O)(=O)N4CCCC(CN5CCC(c6ccc7c(N8CCC(=O)NC8=O)nn(C)c7c6)C(C)C5)C4)cc3F)nc12]

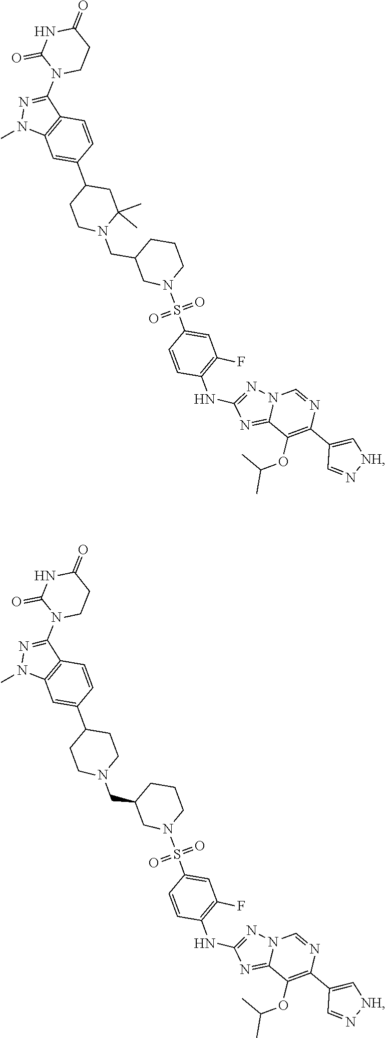 [SMILES: CC(C)Oc1c(-c2cn[nH]c2)ncn2nc(Nc3ccc(S(=O)(=O)N4CCCC(CN5CCC(c6ccc7c(N8CCC(=O)NC8=O)nn(C)c7c6)CC5(C)C)C4)cc3F)nc12 ; CC(C)Oc1c(-c2cn[nH]c2)ncn2nc(Nc3ccc(S(=O)(=O)N4CCC[C@H](CN5CCC(c6ccc7c(N8CCC(=O)NC8=O)nn(C)c7c6)CC5)C4)cc3F)nc12]

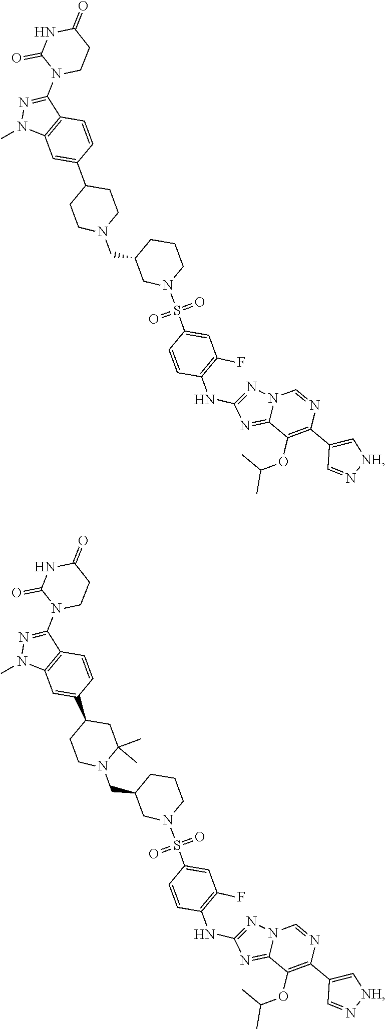 [SMILES: CC(C)Oc1c(-c2cn[nH]c2)ncn2nc(Nc3ccc(S(=O)(=O)N4CCC[C@@H](CN5CCC(c6ccc7c(N8CCC(=O)NC8=O)nn(C)c7c6)CC5)C4)cc3F)nc12 ; CC(C)Oc1c(-c2cn[nH]c2)ncn2nc(Nc3ccc(S(=O)(=O)N4CCC[C@H](CN5CC[C@@H](c6ccc7c(N8CCC(=O)NC8=O)nn(C)c7c6)CC5(C)C)C4)cc3F)nc12]

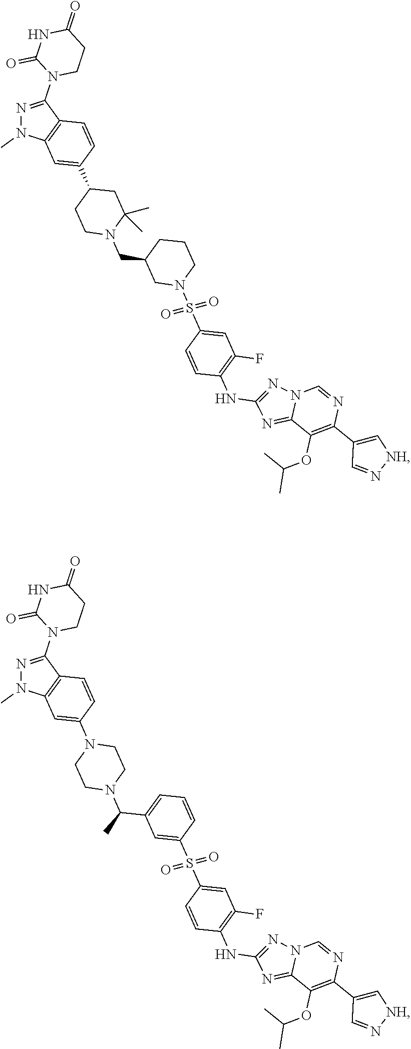 [SMILES: CC(C)Oc1c(-c2cn[nH]c2)ncn2nc(Nc3ccc(S(=O)(=O)N4CCC[C@H](CN5CC[C@H](c6ccc7c(N8CCC(=O)NC8=O)nn(C)c7c6)CC5(C)C)C4)cc3F)nc12 ; CC(C)Oc1c(-c2cn[nH]c2)ncn2nc(Nc3ccc(S(=O)(=O)c4cccc([C@@H](C)N5CCN(c6ccc7c(N8CCC(=O)NC8=O)nn(C)c7c6)CC5)c4)cc3F)nc12]

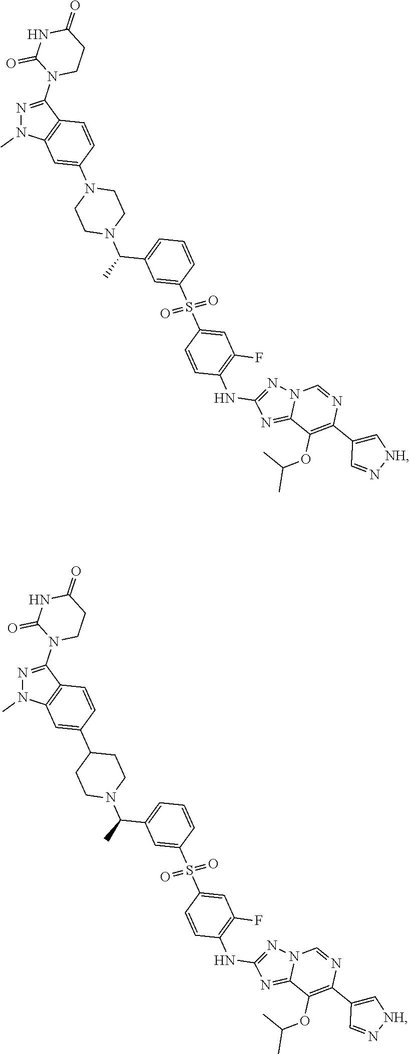 [SMILES: CC(C)Oc1c(-c2cn[nH]c2)ncn2nc(Nc3ccc(S(=O)(=O)c4cccc([C@H](C)N5CCN(c6ccc7c(N8CCC(=O)NC8=O)nn(C)c7c6)CC5)c4)cc3F)nc12 ; CC(C)Oc1c(-c2cn[nH]c2)ncn2nc(Nc3ccc(S(=O)(=O)c4cccc([C@@H](C)N5CCC(c6ccc7c(N8CCC(=O)NC8=O)nn(C)c7c6)CC5)c4)cc3F)nc12]

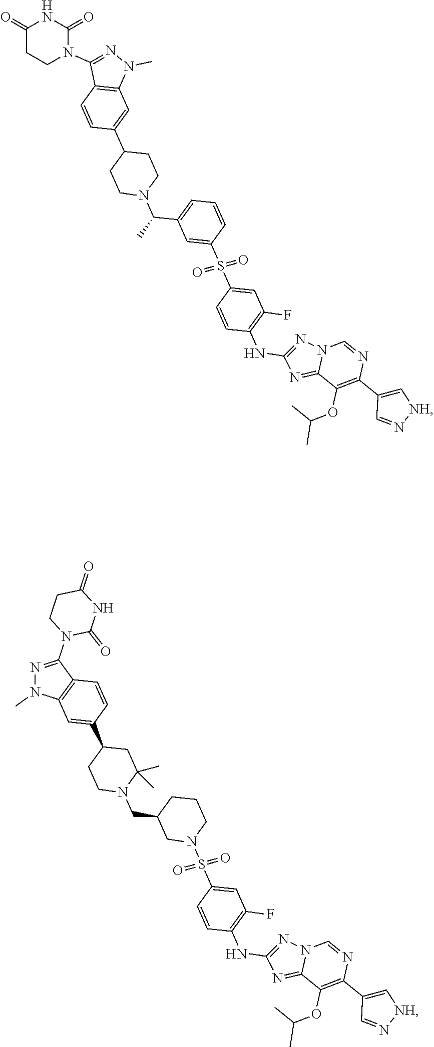 [SMILES: CC(C)Oc1c(-c2cn[nH]c2)ncn2nc(Nc3ccc(S(=O)(=O)c4cccc([C@H](C)N5CCC(c6ccc7c(N8CCC(=O)NC8=O)nn(C)c7c6)CC5)c4)cc3F)nc12 ; CC(C)Oc1c(-c2cn[nH]c2)ncn2nc(Nc3ccc(S(=O)(=O)N4CCC[C@H](CN5CC[C@@H](c6ccc7c(N8CCC(=O)NC8=O)nn(C)c7c6)CC5(C)C)C4)cc3F)nc12]

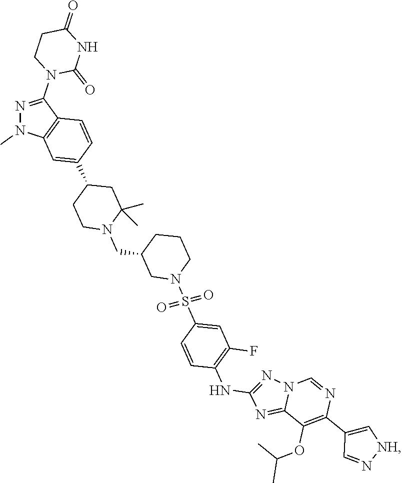 [SMILES: CC(C)Oc1c(-c2cn[nH]c2)ncn2nc(Nc3ccc(S(=O)(=O)N4CCC[C@@H](CN5CC[C@H](c6ccc7c(N8CCC(=O)NC8=O)nn(C)c7c6)CC5(C)C)C4)cc3F)nc12]

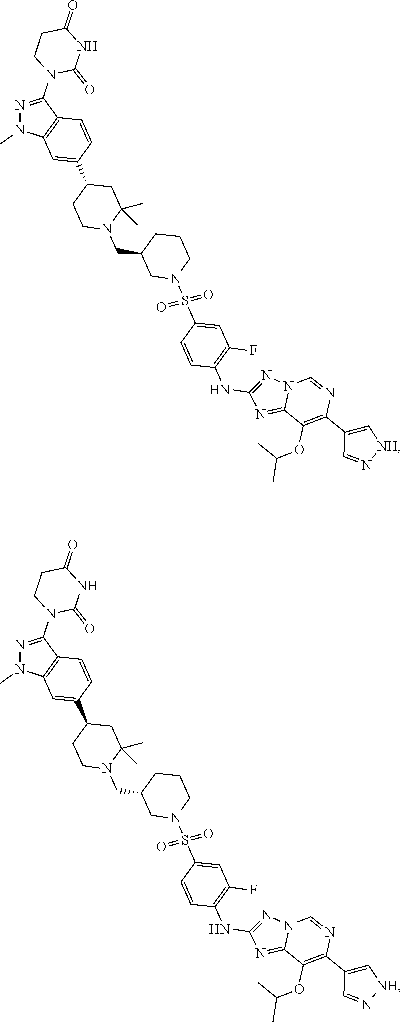 [SMILES: CC(C)Oc1c(-c2cn[nH]c2)ncn2nc(Nc3ccc(S(=O)(=O)N4CCC[C@H](CN5CC[C@H](c6ccc7c(N8CCC(=O)NC8=O)nn(C)c7c6)CC5(C)C)C4)cc3F)nc12 ; CC(C)Oc1c(-c2cn[nH]c2)ncn2nc(Nc3ccc(S(=O)(=O)N4CCC[C@@H](CN5CC[C@@H](c6ccc7c(N8CCC(=O)NC8=O)nn(C)c7c6)CC5(C)C)C4)cc3F)nc12]

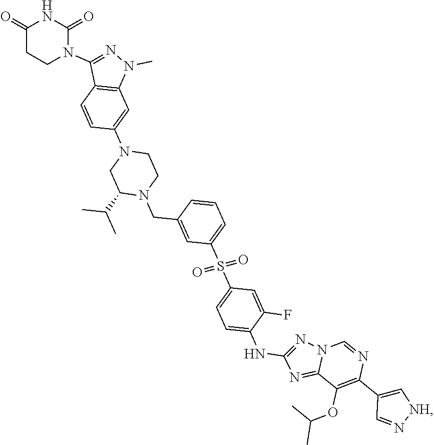 [SMILES: CC(C)Oc1c(-c2cn[nH]c2)ncn2nc(Nc3ccc(S(=O)(=O)c4cccc(CN5CCN(c6ccc7c(N8CCC(=O)NC8=O)nn(C)c7c6)C[C@H]5C(C)C)c4)cc3F)nc12]

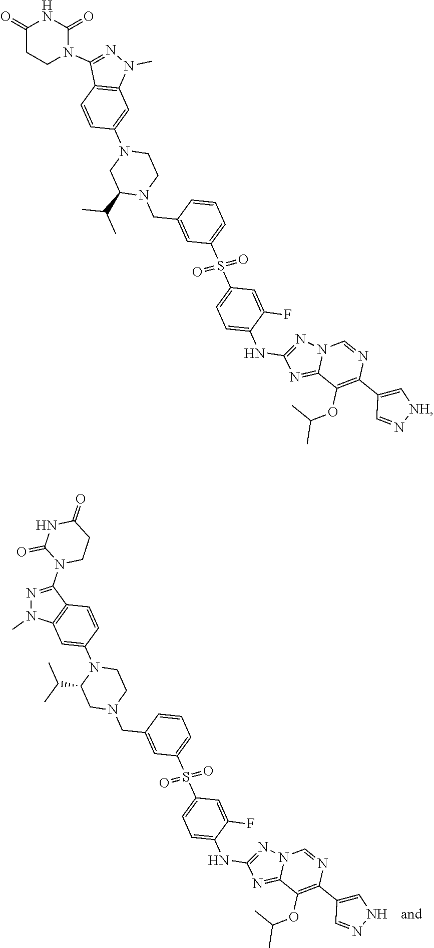 [SMILES: CC(C)Oc1c(-c2cn[nH]c2)ncn2nc(Nc3ccc(S(=O)(=O)c4cccc(CN5CCN(c6ccc7c(N8CCC(=O)NC8=O)nn(C)c7c6)C[C@@H]5C(C)C)c4)cc3F)nc12 ; CC(C)Oc1c(-c2cn[nH]c2)ncn2nc(Nc3ccc(S(=O)(=O)c4cccc(CN5CCN(c6ccc7c(N8CCC(=O)NC8=O)nn(C)c7c6)[C@@H](C(C)C)C5)c4)cc3F)nc12 ; "and"]

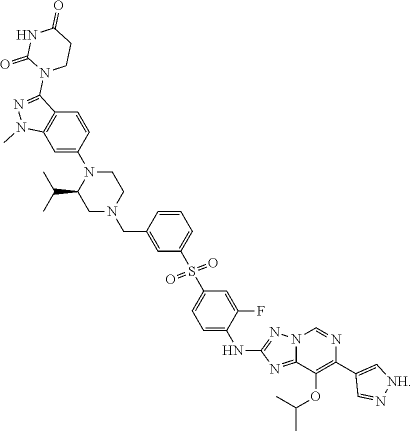 [SMILES: CC(C)Oc1c(-c2cn[nH]c2)ncn2nc(Nc3ccc(S(=O)(=O)c4cccc(CN5CCN(c6ccc7c(N8CCC(=O)NC8=O)nn(C)c7c6)[C@H](C(C)C)C5)c4)cc3F)nc12]

68. (canceled)

69. A method of inhibiting CDK2 and/or CCNE (CCNE1 and/or CCNE2) signaling in a sample, e.g., in vivo or in vitro, by contacting CDK2 and/or CCNE (CCNE1 and/or CCNE2) with a compound of claim 1, or a pharmaceutically acceptable salt thereof.

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)
